SEQUENCE LISTING (927 sequences)

SEQ ID NO: 1 - skipped

SEQ ID NO: 2 - protein, 1367 aa, synthetic construct
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRKLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLKREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 3 - protein, 2117 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLIENSSPSGGSKRTADGSEFEPKKKRKV

SEQ ID NO: 4 - protein, 2117 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSKRTADGSEFEPKKKRKV

SEQ ID NO: 5 - protein, 2131 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRKLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLKREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSKRTADGSEFESPKKKRKVSGGSSGGSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSKRTADGSEFESPKKKRKVGSGPAAKRVKLD

SEQ ID NO: 6 - protein, 1368 aa, Streptococcus pyogenes [sequence omitted]

SEQ ID NO: 7 - protein, 1368 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 8 - protein, 1367 aa, Streptococcus pyogenes
MDKKYSIGLDIGTNSVGWAVITDDYKVPSKKFKVLGNTDRHSIKKNLIGALLFGSGETAEATRLKRTARRRYTRRKNRICYLQEIFSNEMAKVDDSFFHRLEESFLVEEDKKHERHPIFGNIVDEVAYHEKYPTIYHLRKKLADSTDKADLRLIYLALAHMIKFRGHFLIEGDLNPDNSDVDKLFIQLVQIYNQLFEENPINASRVDAKAILSARLSKSRRLENLIAQLPGEKRNGLFGNLIALSLGLTPNFKSNFDLAEDAKLQLSKDTYDDDLDNLLAQIGDQYADLFLAAKNLSDAILLSDILRVNSEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGAYHDLLKIIKDKDFLDNEENEDILEDIVLTLTLFEDRGMIEERLKTYAHLFDDKVMKQLKRRRYTGWGRLSRKLINGIRDKQSGKTILDFLKSDGFANRNFMQLIHDDSLTFKEDIQKAQVSGQGHSLHEQIANLAGSPAIKKGILQTVKIVDELVKVMGHKPENIVIEMARENQTTQKGQKNSRERMKRIEEGIKELGSQILKEHPVENTQLQNEKLYLYYLQNGRDMYVDQELDINRLSDYDVDHIVPQSFIKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 9 - protein, 1404 aa, Streptococcus pyogenes
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGSPKKKRKVSSDYKDHDGDYKDHDIDYKDDDDKAAG

SEQ ID NO: 10 - protein, 1367 aa, synthetic construct
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 11 - protein, 1368 aa, Staphylococcus aureus [sequence omitted]

SEQ ID NO: 12 - protein, 1378 aa, Lactobacillus fermentum
MKEYHIGLDIGTSSIGWAVTDSQFKLMRIKGKTAIGVRLFEEGKTAAERRTFRTTRRRLKRRKWRLHYLDEIFAPHLQEVDENFLRRLKQSNIHPEDPTKNQAFIGKLLFPDLLKKNERGYPTLIKMRDELPVEQRAHYPVMNIYKLREAMINEDRQFDLREVYLAVHHIVKYRGHFLNNASVDKFKVGRIDFDKSFNVLNEAYEELQNGEGSFTIEPSKVEKIGQLLLDTKMRKLDRQKAVAKLLEVKVADKEETKRNKQIATAMSKLVLGYKADFATVAMANGNEWKIDLSSETSEDEIEKFREELSDAQNDILTEITSLFSQIMLNEIVPNGMSISESMMDRYWTHERQLAEVKEYLATQPASARKEFDQVYNKYIGQAPKERGFDLEKGLKKILSKKENWKEIDELLKAGDFLPKQRTSANGVIPHQMHQQELDRIIEKQAKYYPWLATENPATGERDRHQAKYELDQLVSFRIPYYVGPLVTPEVQKATSGAKFAWAKRKEDGEITPWNLWDKIDRAESAEAFIKRMTVKDTYLLNEDVLPANSLLYQKYNVLNELNNVRVNGRRLSVGIKQDIYTELFKKKKTVKASDVASLVMAKTRGVNKPSVEGLSDPKKFNSNLATYLDLKSIVGDKVDDNRYQTDLENIIEWRSVFEDGEIFADKLTEVEWLTDEQRSALVKKRYKGWGRLSKKLLTGIVDENGQRIIDLMWNTDQNFKEIVDQPVFKEQIDQLNQKAITNDGMTLRERVESVLDDAYTSPQNKKAIWQVVRVVEDIVKAVGNAPKSISIEFARNEGNKGEITRSRRTQLQKLFEDQAHELVKDTSLTEELEKAPDLSDRYYFYFTQGGKDMYTGDPINFDEISTKYDIDHILPQSFVKDNSLDNRVLTSRKENNKKSDQVPAKLYAAKMKPYWNQLLKQGLITQRKFENLTKDVDQNIKYRSLGFVKRQLVETRQVIKLTANILGSMYQEAGTEIIETRAGLTKQLREEFDLPKVREVNDYHHAVDAYLTTFAGQYLNRRYPKLRSFFVYGEYMKFKHGSDLKLRNFNFFHELMEGDKSQGKVVDQQTGELITTRDEVAKSFDRLLNMKYMLVSKEVHDRSDQLYGATIVTAKESGKLTSPIEIKKNRLVDLYGAYTNGTSAFMTIIKFTGNKPKYKVIGIPTTSAASLKRAGKPGSESYNQELHRIIKSNPKVKKGFEIVVPHVSYGQLIVDGDCKFTLASPTVQHPATQLVLSKKSLETISSGYKILKDKPAIANERLIRVFDEVVGQMNRYFTIFDQRSNRQKVADARDKFLSLPTESKYEGAKKVQVGKTEVITNLLMGLHANATQGDLKVLGLATFGFFQSTTGLSLSEDTMIVYQSPTGLFERRICLKDI

SEQ ID NO: 13 - protein, 1053 aa, Staphylococcus aureus
MGKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRKLINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYKNDLIKINGELYRVIGVNNDLLNRIEVNMIDITYREYLENMNDKRPPHIIKTIASKTQSIKKYSTDILGNLYEVKSKKHPQIIKK

SEQ ID NO: 14 - protein, 1409 aa, Streptococcus thermophilus
MLFNKCIIISINLDFSNKEKCMTKPYSIGLDIGTNSVGWAVITDNYKVPSKKMKVLGNTSKKYIKKNLLGVLLFDSGITAEGRRLKRTARRRYTRRRNRILYLQEIFSTEMATLDDAFFQRLDDSFLVPDDKRDSKYPIFGNLVEEKVYHDEFPTIYHLRKYLADSTKKADLRLVYLALAHMIKYRGHFLIEGEFNSKNNDIQKNFQDFLDTYNAIFESDLSLENSKQLEEIVKDKISKLEKKDRILKLFPGEKNSGIFSEFLKLIVGNQADFRKCFNLDEKASLHFSKESYDEDLETLLGYIGDDYSDVFLKAKKLYDAILLSGFLTVTDNETEAPLSSAMIKRYNEHKEDLALLKEYIRNISLKTYNEVFKDDTKNGYAGYIDGKTNQEDFYVYLKNLLAEFEGADYFLEKIDREDFLRKQRTFDNGSIPYQIHLQEMRAILDKQAKFYPFLAKNKERIEKILTFRIPYYVGPLARGNSDFAWSIRKRNEKITPWNFEDVIDKESSAEAFINRMTSFDLYLPEEKVLPKHSLLYETFNVYNELTKVRFIAESMRDYQFLDSKQKKDIVRLYFKDKRKVTDKDIIEYLHAIYGYDGIELKGIEKQFNSSLSTYHDLLNIINDKEFLDDSSNEAIIEEIIHTLTIFEDREMIKQRLSKFENIFDKSVLKKLSRRHYTGWGKLSAKLINGIRDEKSGNTILDYLIDDGISNRNFMQLIHDDALSFKKKIQKAQIIGDEDKGNIKEVVKSLPGSPAIKKGILQSIKIVDELVKVMGGRKPESIVVEMARENQYTNQGKSNSQQRLKRLEKSLKELGSKILKENIPAKLSKIDNNALQNDRLYLYYLQNGKDMYTGDDLDIDRLSNYDIDHIIPQAFLKDNSIDNKVLVSSASNRGKSDDFPSLEVVKKRKTFWYQLLKSKLISQRKFDNLTKAERGGLLPEDKAGFIQRQLVETRQITKHVARLLDEKFNNKKDENNRAVRTVKIITLKSTLVSQFRKDFELYKVREINDFHHAHDAYLNAVIASALLKKYPKLEPEFVYGDYPKYNSFRERKSATEKVYFYSNIMNIFKKSISLADGRVIERPLIEVNEETGESVWNKESDLATVRRVLSYPQVNVVKKVEEQNHGLDRGKPKGLFNANLSSKPKPNSNENLVGAKEYLDPKKYGGYAGISNSFAVLVKGTIEKGAKKKITNVLEFQGISILDRINYRKDKLNFLLEKGYKDIELIIELPKYSLFELSDGSRRMLASILSTNNKRGEIHKGNQIFLSQKFVKLLYHAKRISNTINENHRKYVENHKKEFEELFYYILEFNENYVGAKKNGKLLNSAFQSWQNHSIDELCSSFIGPTGSERKGLFELTSRGSAADFEFLGVKIPRYRDYTPSSLLKDATLIHQSVTGLYETRIDLAKLGEG

SEQ ID NO: 15 - protein, 1402 aa, Lactobacillus crispatus
MKIKNYNLALTPSTSAVGHVEVDDDLNILEPVHHQKAIGVAKFGEGETAEARRLARSARRTTKRRANRINHYFNEIMKPEIDKVDPLMFDRIKQAGLSPLDERKEFRTVIFDRPNIASYYHNQFPTIWHLQKYLMITDEKADIRLIYWALHSLLKHRGHFFNTTPMSQFKPGKLNLKDDMLALDDYNDLEGLSFAVANSPEIEKVIKDRSMHKKEKIAELKKLIVNDVPDKDLAKRNNKIITQIVNAIMGNSFHLNFIFDMDLDKLTSKAWSFKLDDPELDTKFDAISGSMTDNQIGIFETLQKIYSAISLLDILNGSSNVVDAKNALYDKHKRDLNLYFKFLNTLPDEIAKTLKAGYTLYIGNRKKDLLAARKLLKVNVAKNFSQDDFYKLINKELKSIDKQGLQTRFSEKVGELVAQNNFLPVQRSSDNVFIPYQLNAITFNKILENQGKYYDFLVKPNPAKKDRKNAPYELSQLMQFTIPYYVGPLVTPEEQVKSGIPKTSRFAWMVRKDNGAITPWNFYDKVDIEATADKFIKRSIAKDSYLLSELVLPKHSLLYEKYEVFNELSNVSLDGKKLSGGVKQILFNEVFKKTNKVNTSRILKALAKHNIPGSKITGLSNPEEFTSSLQTYNAWKKYFPNQIDNFAYQQDLEKMIEWSTVFEDHKILAKKLDEIEWLDDDQKKFVANTRLRGWGRLSKRLLTGLKDNYGKSIMQRLETTKANFQQIVYKPEFREQIDKISQAAAKNQSLEDILANSYTSPSNRKAIRKTMSVVDEYIKLNHGKEPDKIFLMFQRSEQEKGKQTEARSKQLNRILSQLKADKSANKLFSKQLADEFSNAIKKSKYKLNDKQYFYFQQLGRDALTGEVIDYDELYKYTVLHIIPRSKLTDDSQNNKVLTKYKIVDGSVALKFGNSYSDALGMPIKAFWTELNRLKLIPKGKLLNLTTDFSTLNKYQRDGYIARQLVETQQIVKLLATIMQSRFKHTKIIEVRNSQVANIRYQFDYFRIKNLNEYYRGFDAYLAAVVGTYLYKVYPKARRLFVYGQYLKPKKTNQENQDMHLDSEKKSQGFNFLWNLLYGKQDQIFVNGTDVIAFNRKDLITKMNTVYNYKSQKISLAIDYHNGAMFKATLFPRNDRDTAKTRKLIPKKKDYDTDIYGGYTSNVDGYMLLAEIIKRDGNKQYGFYGVPSRLVSELDTLKKTRYTEYEEKLKEIIKPELGVDLKKIKKIKILKNKVPFNQVIIDKGSKFFITSTSYRWNYRQLILSAESQQTLMDLVVDPDFSNHKARKDARKNADERLIKVYEEILYQVKNYMPMFVELHRCYEKLVDAQKTFKSLKISDKAMVLNQILILLHSNATSPVLEKLGYHTRFTLGKKHNLISENAVLVTQSITGLKENHVSIKQML

SEQ ID NO: 16 - protein, 1385 aa, unidentified
  source 1..1385: note = Pedicoccus damnosus
MTNEKYSIGLDIGTSSIGFAVVNDNNRVIRVKGKNAIGVRLFDEGKAAADRRSFRTTRRSFRTTRRRLSRRRWRLKLLREIFDAYITPVDEAFFIRLKESNLSPKDSKKQYSGDILFNDRSDKDFYEKYPTIYHLRNALMTEHRKFDVREIYLAIHHIMKFRGHFLNATPANNFKVGRLNLEEKFEELNDIYQRVFPDESIEFRTDNLEQIKEVLLDNKRSRADRQRTLVSDIYQSSEDKDIEKRNKAVATEILKASLGNKAKLNVITNVEVDKEAAKEWSITFDSESIDDDLAKIEGQMTDDGHEIIEVLRSLYSGITLSAIVPENHTLSQSMVAKYDLHKDHLKLFKKLINGMTDTKKAKNLRAAYDGYIDGVKGKVLPQEDFYKQVQVNLDDSAEANEIQTYIDQDIFMPKQRTKANGSIPHQLQQQELDQIIENQKAYYPWLAELNPNPDKKRQQLAKYKLDELVTFRVPYYVGPMITAKDQKNQSGAEFAWMIRKEPGNITPWNFDQKVDRMATANQFIKRMTTTDTYLLGEDVLPAQSLLYQKFEVLNELNKIRIDHKPISIEQKQQIFNDLFKQFKNVTIKHLQDYLVSQGQYSKRPLIEGLADEKRFNSSLSTYSDLCGIFGAKLVEENDRQEDLEKIIEWSTIFEDKKIYRAKLNDLTWLTDDQKEKLATKRYQGWGRLSRKLLVGLKNSEHRNIMDILWITNENFMQIQAEPDFAKLVTDANKGMLEKTDSQDVINDLYTSPQNKKAIRQILLVVHDIQNAMHGQAPAKIHVEFARGEERNPRRSVQRQRQVEAAYEKVSNELVSAKVRQEFKEAINNKRDFKDRLFLYFMQGGIDIYTGKQLNIDQLSSYQIDHILPQAFVKDDSLTNRVLTNENQVKADSVPIDIFGKKMLSVWGRMKDQGLISKGKYRNLTMNPENISAHTENGFINRQLVETRQVIKLAVNILADEYGDSTQIISVKADLSHQMREDFELLKNRDVNDYHHAFDAYLAAFIGNYLLKRYPKLESYFVYGDFKKFTQKETKMRRFNFIYDLKHCDQVVNKETGEILWTKDEDIKYIRHLFAYKKILVSHEVREKRGALYNQTIYKAKDDKGSGQESKKLIRIKDDKETKIYGGYSGKSLAYMTIVQITKKNKVSYRVIGIPTLALARLNKLENDSTENNGELYKIIKPQFTHYKVDKKNGEIIETTDDFKIVVSKVRFQQLIDDAGQFFMLASDTYKNNAQQLVISNNALKAINNTNITDCPRDDLERLDNLRLDSAFDEIVKKMDKYFSAYDANNFREKIRNSNLIFYQLPVEDQWENNKITELGKRTVLTRILQGLHANATTTDMSIFKIKTPFGQLRQRSGISLSENAQLIYQSPTGLFERRVQLNKIK

SEQ ID NO: 17 - protein, 1367 aa, unidentified
  source 1..1367: note = Fusobaterium nucleatum
MKKQKFSDYYLGFDIGTNSVGWCVTDLDYNVLRFNKKDMWGSRLFEEAKTAAERRVQRNSRRRLKRRKWRLNLLEEIFSNEILKIDSNFFRRLKESSLWLEDKSSKEKFTLFNDDNYKDYDFYKQYPTIFHLRNELIKNPEKKDIRLVYLAIHSIFKSRGHFLFEGQNLKEIKNFETLYNNLIAFLEDNGINKIIDKNNIEKLEKIVCDSKKGLKDKEKEFKEIFNSDKQLVAIFKLSVGSSVSLNDLFDTDEYKKGEVEKEKISFREQIYEDDKPIYYSILGEKIELLDIAKTFYDFMVLNNILADSQYISEAKVKLYEEHKKDLKNLKYIIRKYNKGNYDKLFKDKNENNYSAYIGLNKEKSKKEVIEKSRLKIDDLIKNIKGYLPKVEEIEEKDKAIFNKILNKIELKTILPKQRISDNGTLPYQIHEAELEKILENQSKYYDFLNYEENGIITKDKLLMTFKFRIPYYVGPLNSYHKDKGGNSWIVRKEEGKILPWNFEQKVDIEKSAEEFIKRMTNKCTYLNGEDVIPKDTFLYSEYVILNELNKVQVNDEFLNEENKRKIIDELFKENKKVSEKKFKEYLLVKQIVDGTIELKGVKDSFNSNYISYIRFKDIFGEKLNLDIYKEISEKSILWKCLYGDDKKIFEKKIKNEYGDILTKDEIKKINTFKFNNWGRLSEKLLTGIEFINLETGECYSSVMDALRRTNYNLMELLSSKFTLQESINNENKEMNEASYRDLIEESYVSPSLKRAIFQTLKIYEEIRKITGRVPKKVFIEMARGGDESMKNKKIPARQEQLKKLYDSCGNDIANFSIDIKEMKNSLISYDNNSLRQKKLYLYYLQFGKCMYTGREIDLDRLLQNNDTYDIDHIYPRSKVIKDDSFDNLVLVLKNENAEKSNEYPVKKEIQEKMKSFWRFLKEKNFISDEKYKRLTGKDDFELRGFMARQLVNVRQTTKEVGKILQQIEPEIKIVYSKAEIASSFREMFDFIKVRELNDTHHAKDAYLNIVAGNVYNTKFTEKPYRYLQEIKENYDVKKIYNYDIKNAWDKENSLEIVKKNMEKNTVNITRFIKEKKGQLFDLNPIKKGETSNEIISIKPKVYNGKDDKLNEKYGYYKSLNPAYFLYVEHKEKNKRIKSFERVNLVDVNNIKDEKSLVKYLIENKKLVEPRVIKKVYKRQVILINDYPYSIVTLDSNKLMDFENLKPLFLENKYEKILKNVIKFLEDNQGKSEENYKFIYLKKKDRYEKNETLESVKDRYNLEFNEMYDKFLEKLDSKDYKNYMNNKKYQELLDVKEKFIKLNLFDKAFTLKSFLDLFNRKTMADFSKVGLTKYLGKIQKISSNVLSKNELYLLEESVTGLFVKKIKL

SEQ ID NO: 18 - protein, 1286 aa, Enterococcus cecorum
RRKQRIQILQELLGEEVLKTDPGFFHRMKESRYVVEDKRTLDGKQVELPYALFVDKDYTDKEYYKQFPTINHLIVYLMTTSDTPDIRLVYLALHYYMKNRGNFLHSGDINNVKDINDILEQLDNVLETFLDGWNLKLKSYVEDIKNIYNRDLGRGERKKAFVNTLGAKTKAEKAFCSLISGGSTNLAELFDDSSLKEIETPKIEFASSSLEDKIDGIQEALEDRFAVIEAAKRLYDWKTLTDILGDSSSLAEARVNSYQMHHEQLLELKSLVKEYLDRKVFQEVFVSLNVANNYPAYIGHTKINGKKKELEVKRTKRNDFYSYVKKQVIEPIKKKVSDEAVLTKLSEIESLIEVDKYLPLQVNSDNGVIPYQVKLNELTRIFDNLENRIPVLRENRDKIIKTFKFRIPYYVGSLNGVVKNGKCTNWMVRKEEGKIYPWNFEDKVDLEASAEQFIRRMTNKCTYLVNEDVLPKYSLLYSKYLVLSELNNLRIDGRPLDVKIKQDIYENVFKKNRKVTLKKIKKYLLKEGIITDDDELSGLADDVKSSLTAYRDFKEKLGHLDLSEAQMENIILNITLFGDDKKLLKKRLAALYPFIDDKSLNRIATLNYRDWGRLSERFLSGITSVDQETGELRTIIQCMYETQANLMQLLAEPYHFVEAIEKENPKVDLESISYRIVNDLYVSPAVKRQIWQTLLVIKDIKQVMKHDPERIFIEMAREKQESKKTKSRKQVLSEVYKKAKEYEHLFEKLNSLTEEQLRSKKIYLYFTQLGKCMYSGEPIDFENLVSANSNYDIDHIYPQSKTIDDSFNNIVLVKKSLNAYKSNHYPIDKNIRDNEKVKTLWNTLVSKGLITKEKYERLIRSTPFSDEELAGFIARQLVETRQSTKAVAEILSNWFPESEIVYSKAKNVSNFRQDFEILKVRELNDCHHAHDAYLNIVVGNAYHTKFTNSPYRFIKNKANQEYNLRKLLQKVNKIESNGVVAWVGQSENNPGTIATVKKVIRRNTVLISRMVKEVDGQLFDLTLMKKGKGQVPIKSSDERLTDISKYGGYNKATGAYFTFVKSKKRGKVVRSFEYVPLHLSKQFENNNELLKEYIEKDRGLTDVEILIPKVLINSLFRYNGSLVRITGRGDTRLLLVHEQPLYVSNSFVQQLKSVSSYKLKKSENDNAKLTKTATEKLSNIDELYDGLLRKLDLPIYSYWFSSIKEYLVESRTKYIKLSIEEKALVIFEILHLFQSDAQVPNLKILGLSTKPSRIRIQKNLKDTDKMSIIHQSPSGIFEHEIELTSL

SEQ ID NO: 19 - protein, 1312 aa, unidentified
  source 1..1312: note = Anaerostipes hadrus
MQNGFLGITVSSEQVGWAVTNPKYELERASRKDLWGVRLFDKAETAEDRRMFRTNRRLNQRKKNRIHYLRDIFHEEVNQKDPNFFQQLDESNFCEDDRTVEFNFDTNLYKNQFPTVYHLRKYLMETKDKPDIRLVYLAFSKFMKNRGHFLYKGNLGEVMDFENSMKGFCESLEKFNIDFPTLSDEQVKEVRDILCDHKIAKTVKKKNIITITKVKSKTAKAWIGLFCGCSVPVKVLFQDIDEEIVTDPEKISFEDASYDDYIANIEKGVGIYYEAIVSAKMLFDWSILNEILGDHQLLSDAMIAEYNKHHDDLKRLQKIIKGTGSRELYQDIFINDVSGNYVCYVGHAKTMSSADQKQFYTFLKNRLKNVNGISSEDAEWIDTEIKNGTLLPKQTKRDNSVIPHQLQLREFELILDNMQEMYPFLKENREKLLKIFNFVIPYYVGPLKGVVRKGESTNWMVPKKDGVIHPWNFDEMVDKEASAECFISRMTGNCSYLFNEKVLPKNSLLYETFEVLNELNPLKINGEPISVELKQRIYEQLFLTGKKVTKKSLTKYLIKNGYDKDIELSGIDNEFHSNLKSHIDFEDYDNLSDEEVEQIILRITVFEDKQLLKDYLNREFVKLSEDERKQICSLSYKGWGNLSEMLLNGITVTDSNGVEVSVMDMLWNTNLNLMQILSKKYGYKAEIEHYNKEHEKTIYNREDLMDYLNIPPAQRRKVNQLITIVKSLKKTYGVPNKIFFKISREHQDDPKRTSSRKEQLKYLYKSLKSEDEKHLMKELDELNDHELSNDKVYLYFLQKGRCIYSGKKLNLSRLRKSNYQNDIDYIYPLSAVNDRSMNNKVLTGIQENRADKYTYFPVDSEIQKKMKGFWMELVLQGFMTKEKYFRLSRENDFSKSELVSFIEREISDNQQSGRMIASVLQYYFPESKIVFVKEKLISSFKRDFHLISSYGHNHLQAAKDAYITIVVGNVYHTKFTMDPAIYFKNHKRKDYDLNRLFLENISRDGQIAWESGPYGSIQTVRKEYAQNHIAVTKRVVEVKGGLFKQMPLKKGHGEYPLKTNDPRFGNIAQYGGYTNVTGSYFVLVESMEKGKKRISLEYVPVYLHERLEDDPGHKLLKEYLVDHRKLNHPKILLAKVRKNSLLKIDGFYYRLNGRSGNALILTNAVELIMDDWQTKTANKISGYMKRRAIDKKARVYQNEFHIQELEQLYDFYLDKLKNGVYKNRKNNQAELIHNEKEQFMELKTEDQCVLLTEIKKLFVCSPMQADLTLIGGSKHTGMIAMSSNVTKADFAVIAEDPLGLRNKVIYSHKGEK

SEQ ID NO: 20 - protein, 1344 aa, Kandleria vitulina
MSQNNNKIYNIGLDIGDASVGWAVVDEHYNLLKRHGKHMWGSRLFTQANTAVERRSSRSTRRRYNKRRERIRLLREIMEDMVLDVDPTFFIRLANVSFLDQEDKKDYLKENYHSNYNLFIDKDFNDKTYYDKYPTIYHLRKHLCESKEKEDPRLIYLALHHIVKYRGNFLYEGQKFSMDVSNIEDKMIDVLRQFNEINLFEYVEDRKKIDEVLNVLKEPLSKKHKAEKAFALFDTTKDNKAAYKELCAALAGNKFNVTKMLKEAELHDEDEKDISFKFSDATFDDAFVEKQPLLGDCVEFIDLLHDIYSWVELQNILGSAHTSEPSISAAMIQRYEDHKNDLKLLKDVIRKYLPKKYFEVFRDEKSKKNNYCNYINHPSKTPVDEFYKYIKKLIEKIDDPDVKTILNKIELESFMLKQNSRTNGAVPYQMQLDELNKILENQSVYYSDLKDNEDKIRSILTFRIPYYFGPLNITKDRQFDWIIKKEGKENERILPWNANEIVDVDKTADEFIKRMRNFCTYFPDEPVMAKNSLTVSKYEVLNEINKLRINDHLIKRDMKDKMLHTLFMDHKSISANAMKKWLVKNQYFSNTDDIKIEGFQKENACSTSLTPWIDFTKIFGKINESNYDFIEKIIYDVTVFEDKKILRRRLKKEYDLDEEKIKKILKLKYSGWSRLSKKLLSGIKTKYKDSTRTPETVLEVMERTNMNLMQVINDEKLGFKKTIDDANSTSVSGKFSYAEVQELAGSPAIKRGIWQALLIVDEIKKIMKHEPAHVYIEFARNEDEKERKDSFVNQMLKLYKDYDFEDETEKEANKHLKGEDAKSKIRSERLKLYYTQMGKCMYTGKSLDIDRLDTYQVDHIVPQSLLKDDSIDNKVLVLSSENQRKLDDLVIPSSIRNKMYGFWEKLFNNKIISPKKFYSLIKTEFNEKDQERFINRQIVETRQITKHVAQIIDNHYENTKVVTVRADLSHQFRERYHIYKNRDINDFHHAHDAYIATILGTYIGHRFESLDAKYIYGEYKRIFRNQKNKGKEMKKNNDGFILNSMRNIYADKDTGEIVWDPNYIDRIKKCFYYKDCFVTKKLEENNGTFFNVTVLPNDTNSDKDNTLATVPVNKYRSNVNKYGGFSGVNSFIVAIKGKKKKGKKVIEVNKLTGIPLMYKNADEEIKINYLKQAEDLEEVQIGKEILKNQLIEKDGGLYYIVAPTEIINAKQLILNESQTKLVCEIYKAMKYKNYDNLDSEKIIDLYRLLINKMELYYPEYRKQLVKKFEDRYEQLKVISIEEKCNIIKQILATLHCNSSIGKIMYSDFKISTTIGRLNGRTISLDDISFIAESPTGMYSKKYKL

SEQ ID NO: 21 - protein, 1288 aa, Enterococcus faecalis
MRLFEEGHTAEDRRLKRTARRRISRRRNRLRYLQAFFEEAMTDLDENFFARLQESFLVPEDKKWHRHPIFAKLEDEVAYHETYPTIYHLRKKLADSSEQADLRLIYLALAHIVKYRGHFLIEGKLSTENTSVKDQFQQFMVIYNQTFVNGESRLVSAPLPESVLIEEELTEKASRTKKSEKVLQQFPQEKANGLFGQFLKLMVGNKADFKKVFGLEEEAKITYASESYEEDLEGILAKVGDEYSDVFLAAKNVYDAVELSTILADSDKKSHAKLSSSMIVRFTEHQEDLKKFKRFIRENCPDEYDNLFKNEQKDGYAGYIAHAGKVSQLKFYQYVKKIIQDIAGAEYFLEKIAQENFLRKQRTFDNGVIPHQIHLAELQAIIHRQAAYYPFLKENQEKIEQLVTFRIPYYVGPLSKGDASTFAWLKRQSEEPIRPWNLQETVDLDQSATAFIERMTNFDTYLPSEKVLPKHSLLYEKFMVFNELTKISYTDDRGIKANFSGKEKEKIFDYLFKTRRKVKKKDIIQFYRNEYNTEIVTLSGLEEDQFNASFSTYQDLLKCGLTRAELDHPDNAEKLEDIIKILTIFEDRQRIRTQLSTFKGQFSAEVLKKLERKHYTGWGRLSKKLINGIYDKESGKTILDYLVKDDGVSKHYNRNFMQLINDSQLSFKNAIQKAQSSEHEETLSETVNELAGSPAIKKGIYQSLKIVDELVAIMGYAPKRIVVEMARENQTTSTGKRRSIQRLKIVEKAMAEIGSNLLKEQPTTNEQLRDTRLFLYYMQNGKDMYTGDELSLHRLSHYDIDHIIPQSFMKDDSLDNLVLVGSTENRGKSDDVPSKEVVKDMKAYWEKLYAAGLISQRKFQRLTKGEQGGLTLEDKAHFIQRQLVETRQITKNVAGILDQRYNAKSKEKKVQIITLKASLTSQFRSIFGLYKVREVNDYHHGQDAYLNCVVATTLLKVYPNLAPEFVYGEYPKFQTFKENKATAKAIIYTNLLRFFTEDEPRFTKDGEILWSNSYLKTIKKELNYHQMNIVKKVEVQKGGFSKESIKPKGPSNKLIPVKNGLDPQKYGGFDSPVVAYTVLFTHEKGKKPLIKQEILGITIMEKTRFEQNPILFLEEKGFLRPRVLMKLPKYTLYEFPEGRRRLLASAKEAQKGNQMVLPEHLLTLLYHAKQCLLPNQSESLAYVEQHQPEFQEILERVVDFAEVHTLAKSKVQQIVKLFEANQTADVKEIAASFIQLMQFNAMGAPSTFKFFQKDIERARYTSIKEIFDATIIYQSPTGLYETRRKVVD

SEQ ID NO: 22 - protein, 1052 aa, Staphylococcus aureus
KRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 23 - protein, 1082 aa, Geobacillus thermodenitrificans
MKYKIGLDIGITSIGWAVINLDIPRIEDLGVRIFDRAENPKTGESLALPRRLARSARRRLRRRKHRLERIRRLFVREGILTKEELNKLFEKKHEIDVWQLRVEALDRKLNNDELARILLHLAKRRGFRSNRKSERTNKENSTMLKHIEENQSILSSYRTVAEMVVKDPKFSLHKRNKEDNYTNTVARDDLEREIKLIFAKQREYGNIVCTEAFEHEYISIWASQRPFASKDDIEKKVGFCTFEPKEKRAPKATYTFQSFTVWEHINKLRLVSPGGIRALTDDERRLIYKQAFHKNKITFHDVRTLLNLPDDTRFKGLLYDRNTTLKENEKVRFLELGAYHKIRKAIDSVYGKGAAKSFRPIDFDTFGYALTMFKDDTDIRSYLRNEYEQNGKRMENLADKVYDEELIEELLNLSFSKFGHLSLKALRNILPYMEQGEVYSTACERAGYTFTGPKKKQKTVLLPNIPPIANPVVMRALTQARKVVNAIIKKYGSPVSIHIELARELSQSFDERRKMQKEQEGNRKKNETAIRQLVEYGLTLNPTGLDIVKFKLWSEQNGKCAYSLQPIEIERLLEPGYTEVDHVIPYSRSLDDSYTNKVLVLTKENREKGNRTPAEYLGLGSERWQQFETFVLTNKQFSKKKRDRLLRLHYDENEENEFKNRNLNDTRYISRFLANFIREHLKFADSDDKQKVYTVNGRITAHLRSRWNFNKNREESNLHHAVDAAIVACTTPSDIARVTAFYQRREQNKELSKKTDPQFPQPWPHFADELQARLSKNPKESIKALNLGNYDNEKLESLQPVFVSRMPKRSITGAAHQETLRRYIGIDERSGKIQTVVKKKLSEIQLDKTGHFPMYGKESDPRTYEAIRQRLLEHNNDPKKAFQEPLYKPKKNGELGPIIRTIKIIDTTNQVIPLNDGKTVAYNSNIVRVDVFEKDGKYYCVPIYTIDMMKGILPNKAIEPNKPYSEWKEMTEDYTFRFSLYPNDLIRIEFPREKTIKTAVGEEIKIKDLFAYYQTIDSSNGGLSLVSHDNNFSLRSIGSRTLKRFEKYQVDVLGNIYKVRGEKRVGVASSSHSKAGETIRPL

SEQ ID NO: 24 - protein, 1375 aa, Streptococcus canis
MEKKYSIGLDIGTNSVGWAVITDDYKVPSKKFKVLGNTNRKSIKKNLMGALLFDSGETAEATRLKRTARRRYTRRKNRIRYLQEIFANEMAKLDDSFFQRLEESFLVEEDKKNERHPIFGNLADEVAYHRNYPTIYHLRKKLADSPEKADLRLIYLALAHIIKFRGHFLIEGKLNAENSDVAKLFYQLIQTYNQLFEESPLDEIEVDAKGILSARLSKSKRLEKLIAVFPNEKKNGLFGNIIALALGLTPNFKSNFDLTEDAKLQLSKDTYDDDLDELLGQIGDQYADLFSAAKNLSDAILLSDILRSNSEVTKAPLSASMVKRYDEHHQDLALLKTLVRQQFPEKYAEIFKDDTKNGYAGYVGIGIKHRKRTTKLATQEEFYKFIKPILEKMDGAEELLAKLNRDDLLRKQRTFDNGSIPHQIHLKELHAILRRQEEFYPFLKENREKIEKILTFRIPYYVGPLARGNSRFAWLTRKSEEAITPWNFEEVVDKGASAQSFIERMTNFDEQLPNKKVLPKHSLLYEYFTVYNELTKVKYVTERMRKPEFLSGEQKKAIVDLLFKTNRKVTVKQLKEDYFKKIECFDSVEIIGVEDRFNASLGTYHDLLKIIKDKDFLDNEENEDILEDIVLTLTLFEDREMIEERLKTYAHLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDHIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGD

SEQ ID NO: 25 - DNA, 4104 bp, Streptococcus pyogenes
  source 1..4104: mol_type = genomic DNA
atggataagaaatactcaataggcttagatatcggcacaaatagcgtcggatgggcggtgatcactgatgattataaggttccgtctaaaaagttcaaggttctgggaaatacagaccgccacagtatcaaaaaaaatcttataggggctcttttatttggcagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggcagattctactgataaagcggatttgcgcttaatctatttggccttagcgcatatgattaagtttcgtggtcattttttgattgagggagatttaaatcctgataatagtgatgtggacaaactatttatccagttggtacaaatctacaatcaattatttgaagaaaaccctattaacgcaagtagagtagatgctaaagcgattctttctgcacgattgagtaaatcaagacgattagaaaatctcattgctcagctccccggtgagaagagaaatggcttgtttgggaatctcattgctttgtcattgggattgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatagtgaaataactaaggctcccctatcagcttcaatgattaagcgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgagggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggcgcctaccatgatttgctaaaaattattaaagataaagattttttggataatgaagaaaatgaagatatcttagaggatattgttttaacattgaccttatttgaagatagggggatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagatattcaaaaagcacaggtgtctggacaaggccatagtttacatgaacagattgctaacttagctggcagtcctgctattaaaaaaggtattttacagactgtaaaaattgttgatgaactggtcaaagtaatggggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctacaaaatggaagagacatgtatgtggaccaagaattagatattaatcgtttaagtgattatgatgtcgatcacattgttccacaaagtttcattaaagacgattcaatagacaataaggtactaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactga

SEQ ID NO: 26 - DNA, 4212 bp, Streptococcus pyogenes
  source 1..4212: mol_type = genomic DNA
atggataaaaagtattctattggtttagacatcggcactaattccgttggatgggctgtcataaccgatgaatacaaagtaccttcaaagaaatttaaggtgttggggaacacagaccgtcattcgattaaaaagaatcttatcggtgccctcctattcgatagtggcgaaacggcagaggcgactcgcctgaaacgaaccgctcggagaaggtatacacgtcgcaagaaccgaatatgttacttacaagaaatttttagcaatgagatggccaaagttgacgattctttctttcaccgtttggaagagtccttccttgtcgaagaggacaagaaacatgaacggcaccccatctttggaaacatagtagatgaggtggcatatcatgaaaagtacccaacgatttatcacctcagaaaaaagctagttgactcaactgataaagcggacctgaggttaatctacttggctcttgcccatatgataaagttccgtgggcactttctcattgagggtgatctaaatccggacaactcggatgtcgacaaactgttcatccagttagtacaaacctataatcagttgtttgaagagaaccctataaatgcaagtggcgtggatgcgaaggctattcttagcgcccgcctctctaaatcccgacggctagaaaacctgatcgcacaattacccggagagaagaaaaatgggttgttcggtaaccttatagcgctctcactaggcctgacaccaaattttaagtcgaacttcgacttagctgaagatgccaaattgcagcttagtaaggacacgtacgatgacgatctcgacaatctactggcacaaattggagatcagtatgcggacttatttttggctgccaaaaaccttagcgatgcaatcctcctatctgacatactgagagttaatactgagattaccaaggcgccgttatccgcttcaatgatcaaaaggtacgatgaacatcaccaagacttgacacttctcaaggccctagtccgtcagcaactgcctgagaaatataaggaaatattctttgatcagtcgaaaaacgggtacgcaggttatattgacggcggagcgagtcaagaggaattctacaagtttatcaaacccatattagagaagatggatgggacggaagagttgcttgtaaaactcaatcgcgaagatctactgcgaaagcagcggactttcgacaacggtagcattccacatcaaatccacttaggcgaattgcatgctatacttagaaggcaggaggatttttatccgttcctcaaagacaatcgtgaaaagattgagaaaatcctaacctttcgcataccttactatgtgggacccctggcccgagggaactctcggttcgcatggatgacaagaaagtccgaagaaacgattactccatggaattttgaggaagttgtcgataaaggtgcgtcagctcaatcgttcatcgagaggatgaccaactttgacaagaatttaccgaacgaaaaagtattgcctaagcacagtttactttacgagtatttcacagtgtacaatgaactcacgaaagttaagtatgtcactgagggcatgcgtaaacccgcctttctaagcggagaacagaagaaagcaatagtagatctgttattcaagaccaaccgcaaagtgacagttaagcaattgaaagaggactactttaagaaaattgaatgcttcgattctgtcgagatctccggggtagaagatcgatttaatgcgtcacttggtacgtatcatgacctcctaaagataattaaagataaggacttcctggataacgaagagaatgaagatatcttagaagatatagtgttgactcttaccctctttgaagatcgggaaatgattgaggaaagactaaaaacatacgctcacctgttcgacgataaggttatgaaacagttaaagaggcgtcgctatacgggctggggacgattgtcgcggaaacttatcaacgggataagagacaagcaaagtggtaaaactattctcgattttctaaagagcgacggcttcgccaataggaactttatgcagctgatccatgatgactctttaaccttcaaagaggatatacaaaaggcacaggtttccggacaaggggactcattgcacgaacatattgcgaatcttgctggttcgccagccatcaaaaagggcatactccagacagtcaaagtagtggatgagctagttaaggtcatgggacgtcacaaaccggaaaacattgtaatcgagatggcacgcgaaaatcaaacgactcagaaggggcaaaaaaacagtcgagagcggatgaagagaatagaagagggtattaaagaactgggcagccagatcttaaaggagcatcctgtggaaaatacccaattgcagaacgagaaactttacctctattacctacaaaatggaagggacatgtatgttgatcaggaactggacataaaccgtttatctgattacgacgtcgatcacattgtaccccaatcctttttgaaggacgattcaatcgacaataaagtgcttacacgctcggataagaaccgagggaaaagtgacaatgttccaagcgaggaagtcgtaaagaaaatgaagaactattggcggcagctcctaaatgcgaaactgataacgcaaagaaagttcgataacttaactaaagctgagaggggtggcttgtctgaacttgacaaggccggatttattaaacgtcagctcgtggaaacccgccaaatcacaaagcatgttgcacagatactagattcccgaatgaatacgaaatacgacgagaacgataagctgattcgggaagtcaaagtaatcactttaaagtcaaaattggtgtcggacttcagaaaggattttcaattctataaagttagggagataaataactaccaccatgcgcacgacgcttatcttaatgccgtcgtagggaccgcactcattaagaaatacccgaagctagaaagtgagtttgtgtatggtgattacaaagtttatgacgtccgtaagatgatcgcgaaaagcgaacaggagataggcaaggctacagccaaatacttcttttattctaacattatgaatttctttaagacggaaatcactctggcaaacggagagatacgcaaacgacctttaattgaaaccaatggggagacaggtgaaatcgtatgggataagggccgggacttcgcgacggtgagaaaagttttgtccatgccccaagtcaacatagtaaagaaaactgaggtgcagaccggagggttttcaaaggaatcgattcttccaaaaaggaatagtgataagctcatcgctcgtaaaaaggactgggacccgaaaaagtacggtggcttcgatagccctacagttgcctattctgtcctagtagtggcaaaagttgagaagggaaaatccaagaaactgaagtcagtcaaagaattattggggataacgattatggagcgctcgtcttttgaaaagaaccccatcgacttccttgaggcgaaaggttacaaggaagtaaaaaaggatctcataattaaactaccaaagtatagtctgtttgagttagaaaatggccgaaaacggatgttggctagcgccggagagcttcaaaaggggaacgaactcgcactaccgtctaaatacgtgaatttcctgtatttagcgtcccattacgagaagttgaaaggttcacctgaagataacgaacagaagcaactttttgttgagcagcacaaacattatctcgacgaaatcatagagcaaatttcggaattcagtaagagagtcatcctagctgatgccaatctggacaaagtattaagcgcatacaacaagcacagggataaacccatacgtgagcaggcggaaaatattatccatttgtttactcttaccaacctcggcgctccagccgcattcaagtattttgacacaacgatagatcgcaaacgatacacttctaccaaggaggtgctagacgcgacactgattcaccaatccatcacgggattatatgaaactcggatagatttgtcacagcttgggggtgacggatcccccaagaagaagaggaaagtctcgagcgactacaaagaccatgacggtgattataaagatcatgacatcgattacaaggatgacgatgacaaggctgcagga

SEQ ID NO: 27 - DNA, 4107 bp, Streptococcus pyogenes
  source 1..4107: mol_type = genomic DNA
atggataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactga

SEQ ID NO: 28-32 - skipped (5 entries)

SEQ ID NO: 33 - protein, 677 aa, unidentified
  source 1..677: note = Moloney murine leukemia virus
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLIENSSP

SEQ ID NO: 34 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 35 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPNVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYADDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLIENSSP

SEQ ID NO: 36 - protein, 677 aa, synthetic construct
TLNIEDEYRLHETSKEPDISLGSTWLSDFPQTWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFYEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLIENSSP

SEQ ID NO: 37 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFSEALHRDLADFRIQHPDLILLQYADDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQKIKQALLTAPALGLPDLTKPFELFVDEKQGYAKGVLTQKLGPCRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLIENSSP

SEQ ID NO: 38 - protein, 677 aa, synthetic construct
TLNIEDEYRLHETSKEPDVSLGSTWLSDFPQAWAETGGMGLAVRQAPLIIPLKATSTPVYIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYADDLLLAATSELDCQQGTRALLQTLGSLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLIENSSP

SEQ ID NO: 39 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRLVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYADDLLLAATSELDCQQGTRALLQTLGNLGYRASAKKAQICQKQVKYLGYLLKEGQRWLTEARKEAVMGQPTPKTPRQLREFLGTAGFCRLWIPRFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLIENSSP

SEQ ID NO: 40 - protein, 677 aa, synthetic construct
TLNIEDEYRLHETSKEPDVSLGSTWLSDFPQAWAETGGMGLAVRQAPLIIPLKATSTPVYIKQYPMSQEARLGIKPHIQRLLDQGILVPCQSPWNTPLLPVKKPGTNDYRPVQDLREVNKRVEDIHPTVPNPYNLLSRLPPSHQWYTVLDLKDAFFCLRLHPTSQPLFAFEWRDPEMGISGQLTWTRLPQGFKNSPTLFDEALHRDLADFRIQHPDLILLQYADDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLIENSSP

SEQ ID NO: 41 - protein, 677 aa, synthetic construct
TLNIEDEYRLHETSKEPDVSLGSTWLSDFPQAWAETGGMGLAVRQAPLIIPLKATSTPVYIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQFADDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVNQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLIENSSP

SEQ ID NO: 42 - protein, 677 aa, synthetic construct
TLNIEDEYRLHETSKEPDVSLGSTWLSDFPQAWAETGGMGLAVRQAPLIIPLKATSTPVYIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFFLRLHPTSQPLFAFEWRDPEMGISGQLTWTRLPQGFKNSPTLFDEALHRDLADFRIQHPDLILLQYADDLLLAATSELDCQQGTRALLQILGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLIENSSP

SEQ ID NO: 43 - protein, 604 aa, Mus musculus
VFTLWGRDIMKDIKVRLMTDSPDDSQDLMIGAIESNLFADQISWKSDQPVWLNQWPLKQEKLQALQQLVTEQLQLGHLEESNSPWNTPVFVIKKKSGKWRLLQDLRAVNATMHDMGALQPGLPSPVAVPKGWEIIIIDLQDCFFNIKLHPEDCKRFAFSVPSPNFKRPYQRFQWKVLPQGMKNSPTLCQKFVDKAILTVRDKYQDSYIVHYMDDILLAHPSRSIVDEILTSMIQALNKHGLVVSTEKIQKYDNLKYLGTHIQGDSVSYQKLQIRTDKLRTLNDFQKLLGNINWIRPFLKLTTGELKPLFEILNGDSNPISTRKLTPEACKALQLMNERLSTARVKRLDLSQPWSLCILKTEYTPTACLWQDGVVEWIHLPHISPKVITPYDIFCTQLIIKGRHRSKELFSKDPDYIVVPYTKVQFDLLLQEKEDWPISLLGFLGEVHFHLPKDPLLTFTLQTAIIFPHMTSTTPLEKGIVIFTDGSANGRSVTYIQGREPIIKENTQNTAQQAEIVAVITAFEEVSQPFNLYTDSKYVTGLFPEIETATLSPRTKIYTELKHLQRLIHKRQEKFYIGHIRGHTGLPGPLAQGNAYADSLTRILT

SEQ ID NO: 44 - protein, 572 aa, unidentified
  source 1..572: note = Avian Sarcoma Leukosis Virus
TVALHLAIPLKWKPDHTPVWIDQWPLPEGKLVALTQLVEKELQLGHIEPSLSCWNTPVFVIRKASGSYRLLHDLRAVNAKLVPFGAVQQGAPVLSALPRGWPLMVLDLKDCFFSIPLAEQDREAFAFTLPSVNNQAPARRFQWKVLPQGMTCSPTICQLVVGQVLEPLRLKHPSLRMLHYMDDLLLAASSHDGLEAAGEEVISTLERAGFTISPDKIQREPGVQYLGYKLGSTYVAPVGLVAEPRIATLWDVQKLVGSLQWLRPALGIPPRLMGPFYEQLRGSDPNEAREWNLDMKMAWREIVQLSTTAALERWDPALPLEGAVARCEQGAIGVLGQGLSTHPRPCLWLFSTQPTKAFTAWLEVLTLLITKLRASAVRTFGKEVDILLLPACFREDLPLPEGILLALKGFAGKIRSSDTPSIFDIARPLHVSLKVRVTDHPVPGPTVFTDASSSTHKGVVVWREGPRWEIKEIADSGASVQQLEARAVAMALLLWPTTPTNVVTDSAFVAKMLLKMGQEGVPSTAAAFILEDALSQRSAMAAVLHVRSHSEVPGFFTEGNDVADSQATFQAY

SEQ ID NO: 45 - protein, 667 aa, unidentified
  source 1..667: note = Porcine endogenous retrovirus
TLQLDDEYRLYSPQVKPDQDIQSWLEQFPQAWAETAGMGLAKQVPPQVIQLKASATPVSVRQYPLSREAREGIWPHVQRLIQQGILVPVQSPWNTPLLPVRKPGTNDYRPVQDLREVNKRVQDIHPTVPNPYNLLSALPPERNWYTVLDLKDAFFCLRLHPTSQPLFAFEWRDPGTGRTGQLTWTRLPQGFKNSPTIFDEALHRDLANFRIQHPQVTLLQYVDDLLLAGATKQDCLEGTKALLLELSDLGYRASAKKAQICRREVTYLGYSLRGGQRWLTEARKKTVVQIPAPTTAKQVREFLGTAGFCRLWIPGFATLAAPLYPLTKEKGEFSWAPEHQKAFDAIKKALLSAPALALPDVTKPFTLYVDERKGVARGVLTQTLGPWRRPVAYLSKKLDPVASGWPVCLKAIAAVAILVKDADKLTLGQNITVIAPHALENIVRQPPDRWMTNARMTHYQSLLLTERVTFAPPAALNPATLLPEETDEPVTHDCHQLLIEETGVRKDLTDIPLTGEVLTWFTDGSSYVVEGKRMAGAAVVDGTHTIWASSLPEGTSAQKAELMALTQALRLAEGKSINIYTDSRYAFATAHVHGAIYKQRGLLTSAGREIKNKEEILSLLEALHLPKRLAIIHCPGHQKAKDLISRGNQMADRVAKQAAQAVNLLPI

SEQ ID NO: 46 - protein, 625 aa, unidentified
  source 1..625: note = Human immunodeficiency virus or Moloney murine leukemia virus
PISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDEDFRKYTAFTIPSINNETPGIRYQYNVLPQGWKGSPAIFQSSMTKILEPFKKQNPDIVIYQYMDDLYVGSDLEIGQHRTKIEELRQHLLRWGLTTPDKKHQKEPPFLWMGYELHPDKWTVQPIVLPEKDSWTVNDIQKLVGKLNWASQIYPGIKVRQLCKLLRGTKALTEVIPLTEEAELELAENREILKEPVHGVYYDPSKDLIAEIQKQGQGQWTYQIYQEPFKNLKTGKYARMRGAHTNDVKQLTEAVQKITTESIVIWGKTPKFKLPIQKETWETWWTEYWQATWIPEWEFVNTPPLVKLVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIEN

SEQ ID NO: 47 - protein, 693 aa, Reticuloendotheliosis virus
APLEEEYRLFLEAPIQNVTLLEQWKREIPKVWAEINPPGLASTQAPIHVQLLSTALPVRVRQYPITLEAKRSLRETIRKFRAAGILRPVHSPWNTPLLPVRKSGTSEYRMVQDLREVNKRVETIHPTVPNPYTLLSLLPPDRIWYSVLDLKDAFFCIPLAPESQLIFAFEWADAEEGESGQLTWTRLPQGFKNSPTLFDEALNRDLQGFRLDHPSVSLLQYVDDLLIAADTQAACLSATRDLLMTLAELGYRVSGKKAQLCQEEVTYLGFKIHKGSRSLSNSRTQAILQIPVPKTKRQVREFLGTIGYCRLWIPGFAELAQPLYAATRGGNDPLVWGEKEEEAFQSLKLALTQPPALALPSLDKPFQLFVEETSGAAKGVLTQALGPWKRPVAYLSKRLDPVAAGWPRCLRAIAAAALLTREASKLTFGQDIEITSSHNLESLLRSPPDKWLTNARITQYQVLLLDPPRVRFKQTAALNPATLLPETDDTLPIHHCLDTLDSLTSTRPDLTDQPLAQAEATLFTDGSSYIRDGKRYAGAAVVTLDSVIWAEPLPIGTSAQKAELIALTKALEWSKDKSVNIYTDSRYAFATLHVHGMIYRERGLLTAGGKAIKNAPEILALLTAVWLPKRVAVMHCKGHQKDDAPTSTGNRRADEVAREVAIRPLSTQATISDAPDMPDTETPQYSNVEEALG

SEQ ID NO: 48 - protein, 689 aa, unidentified
  source 1..689: note = Baboon endogenous virus
VSLQDEHRLFDIPVTTSLPDVWLQDFPQAWAETGGLGRAKCQAPIIIDLKPTAVPVSIKQYPMSLEAHMGIRQHIIKFLELGVLRPCRSPWNTPLLPVKKPGTQDYRPVQDLREINKRTVDIHPTVPNPYNLLSTLKPDYSWYTVLDLKDAFFCLPLAPQSQELFAFEWKDPERGISGQLTWTRLPQGFKNSPTLFDEALHRDLTDFRTQHPEVTLLQYVDDLLLAAPTKKACTQGTRHLLQELGEKGYRASAKKAQICQTKVTYLGYILSEGKRWLTPGRIETVARIPPPRNPREVREFLGTAGFCRLWIPGFAELAAPLYALTKESTPFTWQTEHQLAFEALKKALLSAPALGLPDTSKPFTLFLDERQGIAKGVLTQKLGPWKRPVAYLSKKLDPVAAGWPPCLRIMAATAMLVKDSAKLTLGQPLTVITPHTLEAIVRQPPDRWITNARLTHYQALLLDTDRVQFGPPVTLNPATLLPVPENQPSPHDCRQVLAETHGTREDLKDQELPDADHTWYTDGSSYLDSGTRRAGAAVVDGHNTIWAQSLPPGTSAQKAELIALTKALELSKGKKANIYTDSRYAFATAHTHGSIYERRGLLTSEGKEIKNKAEIIALLKALFLPQEVAIIHCPGHQKGQDPVAVGNRQADRVARQAAMAEVLTLATEPDNTSHITIEHTYTSEDQEEA

SEQ ID NO: 49 - protein, 678 aa, Gibbon ape leukemia virus
LNLEEEYRLHEKPVPSSIDPSWLQLFPTVWAERAGMGLANQVPPVVVELRSGASPVAVRQYPMSKEAREGIRPHIQKFLDLGVLVPCRSPWNTPLLPVKKPGTNDYRPVQDLREINKRVQDIHPTVPNPYNLLSSLPPSYTWYSVLDLKDAFFCLRLHPNSQPLFAFEWKDPEKGNTGQLTWTRLPQGFKNSPTLFDEALHRDLAPFRALNPQVVLLQYVDDLLVAAPTYEDCKKGTQKLLQELSKLGYRVSAKKAQLCQREVTYLGYLLKEGKRWLTPARKATVMKIPVPTTPRQVREFLGTAGFCRLWIPGFASLAAPLYPLTKESIPFIWTEEHQQAFDHIKKALLSAPALALPDLTKPFTLYIDERAGVARGVLTQTLGPWRRPVAYLSKKLDPVASGWPTCLKAVAAVALLLKDADKLTLGQNVTVIASHSLESIVRQPPDRWMTNARMTHYQSLLLNERVSFAPPAVLNPATLLPVESEATPVHRCSEILAEETGTRRDLEDQPLPGVPTWYTDGSSFITEGKRRAGAPIVDGKRTVWASSLPEGTSAQKAELVALTQALRLAEGKNINIYTDSRYAFATAHIHGAIYKQRGLLTSAGKDIKNKEEILALLEAIHLPRRVAIIHCPGHQRGSNPVATGNRRADEAAKQAALSTRVLAGTTKPQEPIEPAQEK

SEQ ID NO: 50 - protein, 678 aa, Koala retrovirus
MNLEEEYRLHEKPVPPSIDPSWLQLFPMVWAEKAGMGLANQVPPVVVELKSDASPVAVRQYPMSKEAREGIRPHIQRFLDLGILVPCQSPWNTPLLPVKKPGTNDYRPVQDLREVNKRVQDIHPTVPNPYNLLSSLPPSHTWYSVLDLKDAFFCLKLHPNSQPLFAFEWRDPEKGNTGQLTWTRLPQGFKNSPTLFDEALHRDLASFRALNPQVVMLQYVDDLLVAAPTYRDCKEGTRRLLQELSKLGYRVSAKKAQLCREEVTYLGYLLKGGKRWLTPARKATVMKIPTPTTPRQVREFLGTAGFCRLWIPGFASLAAPLYPLTREKVPFTWTEAHQEAFGRIKEALLSAPALALPDLTKPFALYVDEKEGVARGVLTQTLGPWRRPVAYLSKKLDPVASGWPTCLKAIAAVALLLKDADKLTLGQNVLVIAPHNLESIVRQPPDRWMTNARMTHYQSLLLNERVSFAPPAILNPATLLPVESDDTPIHICSEILAEETGTRPDLRDQPLPGVPAWYTDGSSFIMDGRRQAGAAIVDNKRTVWASNLPEGTSAQKAELIALTQALRLAEGKSINIYTDSRYAFATAHVHGAIYKQRGLLTSAGKDIKNKEEILALLEAIHLPKRVAIIHCPGHQRGTDPVATGNRKADEAAKQAAQSTRILTETTKNQEHFEPTRGK

SEQ ID NO: 51 - protein, 641 aa, Mason-Pfizer monkey virus
MWGRDLLSQMKIMMCSPNDIVTAQMLAQGYSPGKGLGKKENGILHPIPNQGQSNKKGFGNFLTAAIDILAPQQCAEPITWKSDEPVWVDQWPLTNDKLAAAQQLVQEQLEAGHITESSSPWNTPIFVIKKKSGKWRLLQDLRAVNATMVLMGALQPGLPSPVAIPQGYLKIIIDLKDCFFSIPLHPSDQKRFAFSLPSTNFKEPMQRFQWKVLPQGMANSPTLCQKYVATAIHKVRHAWKQMYIIHYMDDILIAGKDGQQVLQCFDQLKQELTAAGLHIAPEKVQLQDPYTYLGFELNGPKITNQKAVIRKDKLQTLNDFQKLLGDINWLRPYLKLTTGDLKPLFDTLKGDSDPNSHRSLSKEALASLEKVETAIAEQFVTHINYSLPLIFLIFNTALTPTGLFWQDNPIMWIHLPASPKKVLLPYYDAIADLIILGRDHSKKYFGIEPSTIIQPYSKSQIDWLMQNTEMWPIACASFVGILDNHYPPNKLIQFCKLHTFVFPQIISKTPLNNALLVFTDGSSTGMAAYTLTDTTIKFQTNLNSAQLVELQALIAVLSAFPNQPLNIYTDSAYLAHSIPLLETVAQIKHISETAKLFLQCQQLIYNRSIPFYIGHVRAHSGLPGPIAQGNQRADLATKIVA

SEQ ID NO: 52 - protein, 571 aa, unidentified
  source 1..571: note = POK11ERV
ATVEPPKPIPLTWKTEKPVWVNQWPLPKQKLEALHLLANEQLEKGHIEPSFSPWNSPVFVIQKKSGKWRMLTDLRAVNAVIQPMGPLQPGLPSPAMIPKDWPLIIIDLKDCFFTIPLAEQDCEKFAFTIPAINNKEPATRFQWKVLPQGMLNSPTICQTFVGRALQPVREKFSDCYIIHYIDDILCAAETKDKLIDCYTFLQAEVANAGLAIASDKIQTSTPFHYLGMQIENRKIKPQKIEIRKDTLKTLNDFQKLLGDINWIRPTLGIPTYAMSNLFSILRGDSDLNSKRILTPEATKEIKLVEEKIQSAQINRIDPLAPLQLLIFATAHSPTGIIIQNTDLVEWSFLPHSTVKTFTLYLDQIATLIGQTRLRIIKLCGNDPDKIVVPLTKEQVRQAFINSGAWQIGLANFVGIIDNHYPKTKIFQFLKMTTWILPKITRREPLENALTVFTDGSSNGKAAYTGPKERVIKTPYQSAQRAELVAVITVLQDFDQPINIISDSAYVVQATRDVETALIKYSMDDQLNQLFNLLQQTVRKRNFPFYITHIRAHTNLPGPLTKANEEADLLVS

SEQ ID NO: 53 - protein, 641 aa, unidentified
  source 1..641: note = Simian retrovirus type 2
MWGRDLLSQMKIMMCSPNDIVTAQMLAQGYSPGKGLGKREDGILQPIPNSGQLDRKGFGNFLATAVDILAPQRYADPITWKSDEPVWVDQWPLTQEKLAAAQQLVQEQLQAGHIIESNSPWNTPIFVIKKKSGKWRLLQDLRAVNATMVLMGALQPGLPSPVAIPQGYFKIVIDLKDCFFTIPLQPVDQKRFAFSLPSTNFKQPMKRYQWKVLPQGMANSPTLCQKYVAAAIEPVRKSWAQMYIIHYMDDILIAGKLGEQVLQCFAQLKQALTTTGLQIAPEKVQLQDPYTYLGFQINGPKITNQKAVIRRDKLQTLNDFQKLLGDINWLRPYLHLTTGDLKPLFDILKGDSNPNSPRSLSEAALASLQKVETAIAEQFVTQIDYTQPLTFLIFNTTLTPTGLFWQNNPVMWVHLPASPKKVLLPYYDAIADLIILGRDNSKKYFGLEPSTIIQPYSKSQIHWLMQNTETWPIACASYAGNIDNHYPPNKLIQFCKLHAVVFPRIISKTPLDNALLVFTDGSSTGIAAYTFEKTTVRFKTSHTSAQLVELQALIAVLSAFPHRALNVYTDSAYLAHSIPLLETVSHIKHISDTAKFFLQCQQLIYNRSIPFYLGHIRAHSGLPGPLSQGNHITDLATKVVA

SEQ ID NO: 54 - protein, 672 aa, unidentified
  source 1..672: note = Wooly Monkey Sarcoma Virus
LNLEEEYRLHEKPVPSSIDPSWLQLFPTVWAERAGMGLANQVPPVVVELRSGASPVAVRQYPMSKEAREGIRPHIQRFLDLGVLVPCQSPWNTPLLPVKKPGTNDYRPVQDLREINKRVQDIHPTVPNPYNLLSSLPPSHTWYSVLDLKDAFFCLKLHPNSQPLFAFEWRDPEKGNTGQLTWTRLPQGFKNSPTLFDEALHRDLAPFRALNPQVVLLQYVDDLLVAAPTYRDCKEGTQKLLQELSKLGYRVSAKKAQLCQKEVTYLGYLLKEGKRWLTPARKATVMKIPPPTTPRQVREFLGTAGFCRLWIPGFASLAAPLYPLTKESIPFIWTEEHQKAFDRIKEALLSAPALALPDLTKPFTLYVDERAGVARGVLTQTLGPWRRPVAYLSKKLDPVASGWPTCLKAVAAVALLLKDADKLTLGQNVTVIASHSLESIVRQPPDRWMTNARMTHYQSLLLNERVSFAPPAVLNPATLLPVESEATPVHRCSEILAEETGTRRDLKDQPLPGVPAWYTDGSSFIAEGKRRAGAAIVDGKRTVWASSLPEGTSAQKAELVALTQALRLAEGKDINIYTDSRYAFATAHIHGAIYKQRGLLTSAGKDIKNKEEILALLEAIHLPKRVAIIHCPGHQKGNDPVATGNRRADEAAKQAALSTRVLAETTKPQELI

SEQ ID NO: 55 - protein, 505 aa, Schizosaccharomyces pombe Tf1 virus
ISSSKHTLSQMNKVSNIVKEPELPDIYKEFKDITADTNTEKLPKPIKGLEFEVELTQENYRLPIRNYPLPPGKMQAMNDEINQGLKSGIIRESKAINACPVMFVPKKEGTLRMVVDYKPLNKYVKPNIYPLPLIEQLLAKIQGSTIFTKLDLKSAYHLIRVRKGDEHKLAFRCPRGVFEYLVMPYGISTAPAHFQYFINTILGEAKESHVVCYMDDILIHSKSESEHVKHVKDVLQKLKNANLIINQAKCEFHQSQVKFIGYHISEKGFTPCQENIDKVLQWKQPKNRKELRQFLGSVNYLRKFIPKTSQLTHPLNKLLKKDVRWKWTPTQTQAIENIKQCLVSPPVLRHFDFSKKILLETDASDVAVGAVLSQKHDDDKYYPVGYYSAKMSKAQLNYSVSDKEMLAIIKSLKHWRHYLESTIEPFKILTDHRNLIGRITNESEPENKRLARWQLFLQDFNFEINYRPGSANHIADALSRIVDETEPIPKDSEDNSINFVNQISI

SEQ ID NO: 56 - protein, 292 aa, Rhodomicrobium vannielii
NSQAQSACCAGANQIVEGATLEKVVAPACLQQAWTRVRKNKGGPGGDGVTIEIFAQNAEVELEKLRAETLAGIYRPRKVRHAIVPKPKGGERKLTIPSVVDRILQTATMLSLGQTVDHHFSSASWAYREGRGVDDALADLRRLRNSGLFWTFDADIMQYFDRILHKRLIDDLFIWVDDLRIVRLIQLWLRSFSYWGRGIAQGAPISPLLANLFLHPMDRLLELEGLASVRYADDFVVLCRSKALAQKAQLIVASHLAARGLKLNMSKTRILAPSEAFIFLGQTVEPVWDTQP

SEQ ID NO: 57 - protein, 312 aa, Vibrio parahaemolyticus
NLVKRLAHHLGKSEPEVIHFLADAPNKYRVYKIPKRSYGHRVIAQPTRELKLYQKAFLELYSFPVHSSATAYCKGKSIKDNALSHVKNHYLLKTDLENFFNSITPNIFWKSIENDSIATPKFSTSEIALVERLIFWRPSKLQGGKLVLSVGAPSSPTISNFCLYQFDEYLSIICKEQNISYTRYADDLTFSTCDKDVLHTVIPLIQSLLDYFFASELKLNHSKTVFSSKAHNRHVTGITLNNEGKLSLGRERKRYIKHLVHSFKYGKLDNTEIRHLQGMLSFAKHIEPIFIDRLKEKYTDELIKIIYEAGHE

SEQ ID NO: 58 - protein, 314 aa, Vibrio cholerae
NILTTLREQLLTNNVIMPQEFERLEVRGSHAYKVYSIPKRKAGRRTIAHPSSKLKICQRHLNAILNPLLKVHDSSYAYVKGRSIKDNALVHSHSAYVLKMDFQNFFNSITPTILRQCLIQNDILLSVNELEKLEQLIFWNPSKKRNGKLILSVGSPISPLISNAIMYPFDKIINDICTKHGINYTRYADDITFSTNIKNTLNKLPEIVEQLIIQTYAGRIIINKRKTVFSSKKHNRHVTGITLTNDSKISIGRSRKRYISSLVFKYINKNLDIDEINHMKGMLAFAYNIEPIYIHRLSHKYKVNIVEKILRGSN

SEQ ID NO: 59 - protein, 407 aa, Escherichia coli
GRPYVTLNLNGMFMDKFKPYSKSNAPITTLEKLSKALSISVEELKAIAELSLDEKYTLKEIPKIDGSKRIVYSLHPKMRLLQSRINKRIFKELVVFPSFLFGSVPSKNDVLNSNVKRDYVSCAKAHCGAKTVLKVDISNFFDNIHRDLVRSVFEEILHIKDEALEYLVDICTKDDFVVQGALTSSYIATLCLFAVEGDVVRRAQRKGLVYTRLVDDITVSSKISNYDFSQMQSHIERMLSEHDLPINKHKTKIFHCSSEPIKVHGLRVDYDSPRLPSDEVKRIRASIHNLKLLAAKNNTKTSVAYRKEFNRCMGRVNKLGRVGHEKYESFKKQLQAIKPMPSKRDVAVIDAAIKSLELSYSKGNQNKHWYKRKYDLTRYKMIILTRSESFKEKLECFKSRLASLKPL

SEQ ID NO: 60 - protein, 419 aa, Geobacillus stearothermophilus
ALLERILARDNLITALKRVEANQGAPGIDGVSTDQLRDYIRAHWSTIHAQLLAGTYRPAPVRRVEIPKPGGGTRQLGIPTVVDRLIQQAILQELTPIFDPDFSSSSFGFRPGRNAHDAVRQAQGYIQEGYRYVVDMDLEKFFDRVNHDILMSRVARKVKDKRVLKLIRAYLQAGVMIEGVKVQTEEGTPQGGPLSPLLANILLDDLDKELEKRGLKFCRYADDCNIYVKSLRAGQRVKQSIQRFLEKTLKLKVNEEKSAVDRPWKRAFLGFSFTPERKARIRLAPRSIQRLKQRIRQLTNPNWSISMPERIHRVNQYVMGWIGYFRLVETPSVLQTIEGWIRRRLRLCQWLQWKRVRTRIRELRALGLKETAVMEIANTRKGAWRTTKTPQLHQALGKTYWTAQGLKSLTQRYFELRQG

SEQ ID NO: 61 - protein, 426 aa, Eubacterium rectale
DTSNLMEQILSSDNLNRAYLQVVRNKGAEGVDGMKYTELKEHLAKNGETIKGQLRTRKYKPQPARRVEIPKPDGGVRNLGVPTVTDRFIQQAIAQVLTPIYEEQFHDHSYGFRPNRCAQQAILTALNIMNDGNDWIVDIDLEKFFDTVNHDKLMTLIGRTIKDGDVISIVRKYLVSGIMIDDEYEDSIVGTPQGGNLSPLLANIMLNELDKEMEKRGLNFVRYADDCIIMVGSEMSANRVMRNISRFIEEKLGLKVNMTKSKVDRPSGLKYLGFGFYFDPRAHQFKAKPHAKSVAKFKKRMKELTCRSWGVSNSYKVEKLNQLIRGWINYFKIGSMKTLCKELDSRIRYRLRMCIWKQWKTPQNQEKNLVKLGIDRNTARRVAYTGKRIAYVCNKGAVNVAISNKRLASFGLISMLDYYIEKCVTC

SEQ ID NO: 62 - protein, 513 aa, Nannocystis exedens
AGQPTSREALYERIRSTSKEEVILEEMIRLGFWPAQGAVPHDPAEEIRRRGELERQLSELREKSRKLYNEKALIAEQRKQRLAESRRKQKETKARRERERQERAQKWAQRKAGEILFLGEDVSGGMSHKTCDAELIKREGVPAIASAEELARAMGIALKELRFLAYNRKVSRVTHYRRFLLPKKTGGLRLISAPMPRLKRAQAWALEHIFNKLSFEPAAHGFVAGRSIVSNARPHVGADVVVNLDLKDFFPTVSFPRVKGALRHLGYSESVATALALVCTEPEVDEVGLDGTTWYVARGERFLPQGSPCSPAITNLLCRRLDRRLHGLAQALGFVYTRYADDLTFSGRGEAAESKRVGKLLRGAADIVAHEGFVVHPDKTRVMRRGRRQEVTGVVVNDKTSVPRDELRKFRATLYQIEKDGPADKRWGNGGDVLAAVHGYACFVAMVDPSRGQPLLARARALLAKHGGPSKPPGGSGPRAPTPVQPTANAPEAPKPVAPATPAAPAKKGWKLF

SEQ ID NO: 63 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLIENSSP

SEQ ID NO: 64 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLIENSSP

SEQ ID NO: 65 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 66 - protein, 677 aa, synthetic construct
TLNIEDEYRLHETSKEPDVSLGSTWLSDFPQAWAETGGMGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 67 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGTAGFCRLWIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 68 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSP

SEQ ID NO: 69 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 70 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 71 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSKARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 72 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLWIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 73 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 74 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTGGSSLLQEGQRKAGAAVTTETEVIWAKALPAGTSAQRAQLIALTQALKMAEGKKLNVYTNSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 75 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGTAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 76 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSKGKEIKNKDEILALLKALFLPKRLSIIHCPGHQKGHSAEARGNRMADQAARKAAITETPDTSTLLIENSSP

SEQ ID NO: 77 - protein, 677 aa, synthetic construct
TLNIEDEYRLHETSKEPDVSLGSTWLSDFPQAWAETGGMGLAVRQAPLIILLKATSTPVSIKQYPMKQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPALFDEALRRDLADFRIQHPDLILLQYVDDLLLAATSELDCQQGTRALLQTLGNLGYRASAKKAQICQKQVKYLGYLLKEGQRWLTEARKETVMGQPTPKTPRQLRKFLGTAGNCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPPDRWLSKARMTHYQALLLDTDRVQFGPVVALNPATLLPLPEEGLQHNCLDILAEAHGTRPDLTDQPLPDADHTWYTGGSSLLQEGQRKAGAAVTTETEVIWAKALPAGTSAQRAELIALTQALKMAEGKKLNVYTNSRYAFATAHIQGEIYRRRGLLTSEGKEIKNKDEILALLKALFLPKRLSIIHCPGHQKGHSAEARGNRMANQAARKAAITETPDTSTLLIENSSP

SEQ ID NO: 78 - protein, 677 aa, synthetic construct
TLNIEDEYRLHETSKEPDVSLGSTWLSDFPQAWAETGGMGLAVRQAPLIILLKATSTPVSIKQYPMKQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPALFNEALRRDLADFRIQHPDLILLQYVDDLLLAATSELDCQQGTRALLQTLGNLGYRASAKKAQICQKQVKYLGYLLKEGQRWLTEARKETVMGQPTPKTPRQLRKFLGTAGNCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPPDRWLSKARMTHYQALLLDTDRVQFGPVVALNPATLLPLPEEGLQHNCLDILAEAHGTRPDLTDQPLPDADHTWYTGGSSLLQEGQRKAGAAVTTETEVIWAKALPAGTSAQRAELIALTQALKMAEGKKLNVYTNSRYAFATAHIQGEIYRRRGLLTSEGKEIKNKDEILALLKALFLPKRLSIIHCPGHQKGHSAEARGNRMANQAARKAAITETPDTSTLLIENSSP

SEQ ID NO: 79 - protein, 34 aa, synthetic construct
SGGSSGGSKRTADGSEFESPKKKRKVSGGSSGGS

SEQ ID NO: 80 - protein, 33 aa, synthetic construct
SGGSSGGSSGSETPGTSESATPESSGGSSGGSS

SEQ ID NO: 81 - protein, 120 aa, synthetic construct
SGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGS

SEQ ID NO: 82 - protein, 4 aa, synthetic construct
SGGS

SEQ ID NO: 83 - protein, 60 aa, synthetic construct
SGGSSGGSSGSETPGTSESATPESAGSYPYDVPDYAGSAAPAAKKKKLDGSGSGGSSGGS

SEQ ID NO: 84 - protein, 150 aa, synthetic construct
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 85 - protein, 30 aa, synthetic construct
GGGGGGGGGGGGGGGGGGGGGGGGGGGGGG

SEQ ID NO: 86 - protein, 150 aa, synthetic construct
EAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAK

SEQ ID NO: 87 - protein, 90 aa, synthetic construct
GGSGGSGGSGGSGGSGGSGGSGGSGGSGGSGGSGGSGGSGGSGGSGGSGGSGGSGGSGGSGGSGGSGGSGGSGGSGGSGGSGGSGGSGGS

SEQ ID NO: 88 - protein, 60 aa, synthetic construct
XPXPXPXPXPXPXPXPXPXPXPXPXPXPXPXPXPXPXPXPXPXPXPXPXPXPXPXPXPXP

SEQ ID NO: 89 - protein, 16 aa, synthetic construct
SGSETPGTSESATPES

SEQ ID NO: 90 - protein, 32 aa, synthetic construct
SGGSSGGSSGSETPGTSESATPESSGGSSGGS

SEQ ID NO: 91 - protein, 10 aa, synthetic construct
SGGSGGSGGS

SEQ ID NO: 92 - protein, 6 aa, synthetic construct
GGSGGS

SEQ ID NO: 93 - protein, 9 aa, synthetic construct
GGSGGSGGS

SEQ ID NO: 94 - protein, 7 aa, synthetic construct
PKKKRKV

SEQ ID NO: 95 - protein, 19 aa, synthetic construct
MKRTADGSEFESPKKKRKV

SEQ ID NO: 96 - protein, 21 aa, synthetic construct
SGGSKRTADGSEFEPKKKRKV

SEQ ID NO: 97 - protein, 18 aa, synthetic construct
KRTADGSEFESPKKKRKV

SEQ ID NO: 98 - protein, 9 aa, synthetic construct
PAAKRVKLD

SEQ ID NO: 99 - protein, 30 aa, synthetic construct
MDSLLMNRRKFLYQFKNVRWAKGRRETYLC

SEQ ID NO: 100 - protein, 20 aa, synthetic construct
AVKRPAATKKAGQAKKKKLD

SEQ ID NO: 101 - protein, 25 aa, synthetic construct
MSRRRKANPTKLSENAKKLAKEVEN

SEQ ID NO: 102 - protein, 9 aa, synthetic construct
KLKIKRPVK

SEQ ID NO: 103 - protein, 8 aa, synthetic construct
VSRKRPRP

SEQ ID NO: 104 - protein, 10 aa, synthetic construct
EGAPPAKRAR

SEQ ID NO: 105 - protein, 12 aa, synthetic construct
PPQPKKKPLDGE

SEQ ID NO: 106 - protein, 17 aa, synthetic construct
KRTADGSEFEPKKKRKV

SEQ ID NO: 107 - protein, 19 aa, synthetic construct
NLSKRPAAIKKAGQAKKKK

SEQ ID NO: 108 - protein, 11 aa, synthetic construct
RQRRNELKRSF

SEQ ID NO: 109 - protein, 38 aa, synthetic construct
NQSSNFGPMKGGNFGGRSSGPYGGGGQYFAKPRNQGGY

SEQ ID NO: 110 - protein, 16 aa, unidentified
  source 1..16: note = Xenopus nucleoplasmin
KRXXXXXXXXXXKKKL

SEQ ID NO: 111 - skipped

SEQ ID NO: 112 - protein, 380 aa, Homo sapiens
MGIQGLAKLIADVAPSAIRENDIKSYFGRKVAIDASMSIYQFLIAVRQGGDVLQNEEGETTSHLMGMFYRTIRMMENGIKPVYVFDGKPPQLKSGELAKRSERRAEAEKQLQQAQAAGAEQEVEKFTKRLVKVTKQHNDECKHLLSLMGIPYLDAPSEAEASCAALVKAGKVYAAATEDMDCLTFGSPVLMRHLTASEAKKLPIQEFHLSRILQELGLNQEQFVDLCILLGSDYCESIRGIGPKRAVDLIQKHKSIEEIVRRLDPNKYPVPENWLHKEAHQLFLEPEVLDPESVELKWSEPNEEELIKFMCGEKQFSEERIRSGVKRLSKSRQGSTQGRLDDFFKVTGSLSSAKRKEPEPKGSTKKKAKTGAAGKFKRGK

SEQ ID NO: 113 - protein, 380 aa, synthetic construct
MGIQGLAKLIADVAPSAIRENDIKSYFGRKVAIDASMSIYQFLIAVRQGGDVLQNEEGETTSHLMGMFYRTIRMMENGIKPVYVFDGKPPQLKSGELAKRSERRAEAEKQLQQAQAAGAEQEVEKFTKRLVKVTKQHNDECKHLLSLMGIPYLDAPSEAEASCAALVRAGKVYAAATEDMDCLTFGSPVLMRHLTASEAKKLPIQEFHLSRILQELGLNQEQFVDLCILLGSDYCESIRGIGPKRAVDLIQKHKSIEEIVRRLDPNKYPVPENWLHKEAHQLFLEPEVLDPESVELKWSEPNEEELIKFMCGEKQFSEERIRSGVKRLSKSRQGSTQGRLDDFFKVTGSLSSAKRKEPEPKGSTKKKAKTGAAGKFKRGK

SEQ ID NO: 114 - protein, 380 aa, synthetic construct
MGIQGLAKLIADVAPSAIRENDIKSYFGRKVAIDASMSIYQFLIAVRQGGDVLQNEEGETTSHLMGMFYRTIRMMENGIKPVYVFDGKPPQLKSGELAKRSERRAEAEKQLQQAQAAGAEQEVEKFTKRLVKVTKQHNDECKHLLSLMGIPYLDAPSEAEASCAALVKAGKVYAAATEDMDCLTFGAPVLMRHLTASEAKKLPIQEFHLSRILQELGLNQEQFVDLCILLGSDYCESIRGIGPKRAVDLIQKHKSIEEIVRRLDPNKYPVPENWLHKEAHQLFLEPEVLDPESVELKWSEPNEEELIKFMCGEKQFSEERIRSGVKRLSKSRQGSTQGRLDDFFKVTGSLSSAKRKEPEPKGSTKKKAKTGAAGKFKRGK

SEQ ID NO: 115 - protein, 380 aa, synthetic construct
MGIQGLAKLIADVAPSAIRENDIKSYFGRKVAIDASMSIYQFLIAVRQGGDVLQNEEGETTSHLMGMFYRTIRMMENGIKPVYVFDGKPPQLKSGELAKRSERRAEAEKQLQQAQAAGAEQEVEKFTKRLVKVTKQHNDECKHLLSLMGIPYLDAPSEAEASCAALVKAGKVYAAATEDMDCLTFGSPVLMRHLTASEAKKLPIQEFHLSRILQELGLNQEQFVDLCILLGSDYCESIRGIGPKRAVDLIQKHKSIEEIVRRLDPNKYPVPENWLHKEAHQLFLEPEVLDPESVELKWSEPNEEELIKFMCGEKQFSEERIRSGVKRLSKSRQGSTQGRLDDFFKVTGSLSSARRKEPEPKGSTKKKAKTGAAGKFKRGK

SEQ ID NO: 116 - protein, 908 aa, synthetic construct
MGVNDLWQILEPVKQHIPLRNLGGKTIAVDLSLWVCEAQTVKKMMGSVMKPHLRNLFFRISYLTQMDVKLVFVMEGEPPKLKADVISKRNQSRYGSSGKSWSQKTGRSHFKSVLRECLHMLECLGIPWVQAAGEAEAMCAYLNAGGHVDGCLTNDGDTFLYGAQTVYRNFTMNTKDPHVDCYTMSSIKSKLGLDRDALVGLAILLGCDYLPKGVPGVGKEQALKLIQILKGQSLLQRFNRWNETSCNSSPQLLVTKKLAHCSVCSHPGSPKDHERNGCRLCKSDKYCEPHDYEYCCPCEWHRTEHDRQLSEVENNIKKKACCCEGFPFHEVIQEFLLNKDKLVKVIRYQRPDLLLFQRFTLEKMEWPNHYACEKLLVLLTHYDMIERKLGSRNSNQLQPIRIVKTRIRNGVHCFEIEWEKPEHYAMEDKQHGEFALLTIEEESLFEAAYPEIVAVYQKQKLEIKGKKQKRIKPKENNLPEPDEVMSFQSHMTLKPTCEIFHKQNSKLNSGISPDPTLPQESISASLNSLLLPKNTPCLNAQEQFMSSLRPLAIQQIKAVSKSLISESSQPNTSSHNISVIADLHLSTIDWEGTSFSNSPAIQRNTFSHDLKSEVESELSAIPDGFENIPEQLSCESERYTANIKKVLDEDSDGISPEEHLLSGITDLCLQDLPLKERIFTKLSYPQDNLQPDVNLKTLSILSVKESCIANSGSDCTSHLSKDLPGIPLQNESRDSKILKGDQLLQEDYKVNTSVPYSVSNTVVKTCNVRPPNTALDHSRKVDMQTTRKILMKKSVCLDRHSSDEQSAPVFGKAKYTTQRMKHSSQKHNSSHFKESGHNKLSSPKIHIKETEQCVRSYETAENEESCFPDSTKSSLSSLQCHKKENNSGTCLDSPLPLRQRLKLRFQST

SEQ ID NO: 117 - protein, 1186 aa, synthetic construct
MGVQGLWKLLECSGRQVSPEALEGKILAVDISIWLNQALKGVRDRHGNSIENPHLLTLFHRLCKLLFFRIRPIFVFDGDAPLLKKQTLVKRRQRKDLASSDSRKTTEKLLKTFLKRQAIKTAFRSKRDEALPSLTQVRRENDLYVLPPLQEEEKHSSEEEDEKEWQERMNQKQALQEEFFHNPQAIDIESEDFSSLPPEVKHEILTDMKEFTKRRRTLFEAMPEESDDFSQYQLKGLLKKNYLNQHIEHVQKEMNQQHSGHIRRQYEDEGGFLKEVESRRVVSEDTSHYILIKGIQAKTVAEVDSESLPSSSKMHGMSFDVKSSPCEKLKTEKEPDATPPSPRTLLAMQAALLGSSSEEELESENRRQARGRNAPAAVDEGSISPRTLSAIKRALDDDEDVKVCAGDDVQTGGPGAEEMRINSSTENSDEGLKVRDGKGIPFTATLASSSVNSAEEHVASTNEGREPTDSVPKEQMSLVHVGTEAFPISDESMIKDRKDRLPLESAVVRHSDAPGLPNGRELTPASPTCTNSVSKNETHAEVLEQQNELCPYESKFDSSLLSSDDETKCKPNSASEVIGPVSLQETSSIVSVPSEAVDNVENVVSFNAKEHENFLETIQEQQTTESAGQDLISIPKAVEPMEIDSEESESDGSFIEVQSVISDEELQAEFPETSKPPSEQGEEELVGTREGEAPAESESLLRDNSERDDVDGEPQEAEKDAEDSLHEWQDINLEELETLESNLLAQQNSLKAQKQQQERIAATVTGQMFLESQELLRLFGIPYIQAPMEAEAQCAILDLTDQTSGTITDDSDIWLFGARHVYRNFFNKNKFVEYYQYVDFHNQLGLDRNKLINLAYLLGSDYTEGIPTVGCVTAMEILNEFPGHGLEPLLKFSEWWHEAQKNPKIRPNPHDTKVKKKLRTLQLTPGFPNPAVAEAYLKPVVDDSKGSFLWGKPDLDKIREFCQRYFGWNRTKTDESLFPVLKQLDAQQTQLRIDSFFRLAQQEKEDAKRIKSQRLNRAVTCMLRKEKEAAASEIEAVSVAMEKEFELLDKAKRKTQKRGITNTLEESSSLKRKRLSDSKRKNTCGGFLGETCLSESSDGSSSEDAESSSLMNVQRRTAAKEPKTSASDSQNSVKEAPVKNGGATTSSSSDSDDDGGKEKMVLVTARSVFGKKRRKLRRARGRKRKT

SEQ ID NO: 118 - protein, 236 aa, Homo sapiens
MSEAPRAETFVFLDLEATGLPSVEPEIAELSLFAVHRSSLENPEHDESGALVLPRVLDKLTLCMCPERPFTAKASEITGLSSEGLARCRKAGFDGAVVRTLQAFLSRQAGPICLVAHNGFDYDFPLLCAELRRLGARLPRDTVCLDTLPALRGLDRAHSHGTRARGRQGYSLGSLFHRYFRAEPSAAHSAEGDVHTLLLIFLHRAAELLAWADEQARGWAHIEPMYLPPDDPSLEA

SEQ ID NO: 119 - protein, 236 aa, Mus musculus
MSEPPRAETFVFLDLEATGLPNMDPEIAEISLFAVHRSSLENPERDDSGSLVLPRVLDKLTLCMCPERPFTAKASEITGLSSESLMHCGKAGFNGAVVRTLQGFLSRQEGPICLVAHNGFDYDFPLLCTELQRLGAHLPQDTVCLDTLPALRGLDRAHSHGTRAQGRKSYSLASLFHRYFQAEPSAAHSAEGDVHTLLLIFLHRAPELLAWADEQARSWAHIEPMYVPPDGPSLEA

SEQ ID NO: 120 - protein, 236 aa, Rattus norvegicus
MSEPLRAETFVFLDLEATGLPNMDPEIAEISLFAVHRSSLENPERDDSGSLVLPRVLDKLTLCMCPERPFTAKASEITGLSSEGLMNCRKAAFNDAVVRTLQGFLSRQEGPICLVAHNGFDYDFPLLCTELQRLGAHLPRDTVCLDTLPALRGLDRVHSHGTRAQGRKSYSLASLFHRYFQAEPSAAHSAEGDVNTLLLIFLHRAPELLAWADEQARSWAHIEPMYVPPDGPSLEA

SEQ ID NO: 121 - protein, 803 aa, Homo sapiens
MGIQGLLQFIKEASEPIHVRKYKGQVVAVDTYCWLHKGAIACAEKLAKGEPTDRYVGFCMKFVNMLLSHGIKPILVFDGCTLPSKKEVERSRRERRQANLLKGKQLLREGKVSEARECFTRSINITHAMAHKVIKAARSQGVDCLVAPYEADAQLAYLNKAGIVQAIITEDSDLLAFGCKKVILKMDQFGNGLEIDQARLGMCRQLGDVFTEEKFRYMCILSGCDYLSSLRGIGLAKACKVLRLANNPDIVKVIKKIGHYLKMNITVPEDYINGFIRANNTFLYQLVFDPIKRKLIPLNAYEDDVDPETLSYAGQYVDDSIALQIALGNKDINTFEQIDDYNPDTAMPAHSRSHSWDDKTCQKSANVSSIWHRNYSPRPESGTVSDAPQLKENPSTVGVERVISTKGLNLPRKSSIVKRPRSAELSEDDLLSQYSLSFTKKTKKNSSEGNKSLSFSEVFVPDLVNGPTNKKSVSTPPRTRNKFATFLQRKNEESGAVVVPGTRSRFFCSSDSTDCVSNKVSIQPLDETAVTDKENNLHESEYGDQEGKRLVDTDVARNSSDDIPNNHIPGDHIPDKATVFTDEESYSFESSKFTRTISPPTLGTLRSCFSWSGGLGDFSRTPSPSPSTALQQFRRKSDSPTSLPENNMSDVSQLKSEESSDDESHPLREEACSSQSQESGEFSLQSSNASKLSQCSSKDSDSEESDCNIKLLDSQSDQTSKLRLSHFSKKDTPLRNKVPGLYKSSSADSLSTTKIKPLGPARASGLSKKPASIQKRKHHNAENKPGLQIKLNELWKNFGFKKF

SEQ ID NO: 122 - protein, 846 aa, Homo sapiens
MGIQGLLQFIKEASEPIHVRKYKGQVVAVDTYCWLHKGAIACAEKLAKGEPTDRYVGFCMKFVNMLLSHGIKPILVFDGCTLPSKKEVERSRRERRQANLLKGKQLLREGKVSEARECFTRSINITHAMAHKVIKAARSQGVDCLVAPYEADAQLAYLNKAGIVQAIITEDSDLLAFGCKKVILKMDQFGNGLEIDQARLGMCRQLGDVFTEEKFRYMCILSGCDYLSSLRGIGLAKACKVLRLANNPDIVKVIKKIGHYLKMNITVPEDYINGFIRANNTFLYQLVFDPIKRKLIPLNAYEDDVDPETLSYAGQYVDDSIALQIALGNKDINTFEQIDDYNPDTAMPAHSRSHSWDDKTCQKSANVSSIWHRNYSPRPESGTVSDAPQLKENPSTVGVERVISTKGLNLPRKSSIVKRPRSAELSEDDLLSQYSLSFTKKTKKNSSEGNKSLSFSEVFVPDLVNGPTNKKSVSTPPRTRNKFATFLQRKNEESGAVVVPGTRSRFFCSSDSTDCVSNKVSIQPLDETAVTDKENNLHESEYGDQEGKRLVDTDVARNSSDDIPNNHIPGDHIPDKATVFTDEESYSFESSKFTRTISPPTLGTLRSCFSWSGGLGDFSRTPSPSPSTALQQFRRKSDSPTSLPENNMSDVSQLKSEESSDDESHPLREEACSSQSQESGEFSLQSSNASKLSQCSSKDSDSEESDCNIKLLDSQSDQTSKLRLSHFSKKDTPLRNKVPGLYKSSSADSLSTTKIKPLGPARASGLSKKPASIQKRKHHNAENKPGLQIKLNELWKNFGFKKDSEKLPPCKKPLSPVRDNIQLTPEAEEDIFNKPECGRVQRAIFQ

SEQ ID NO: 123 - protein, 845 aa, Homo sapiens
MGIQGLLQFIKEASEPIHVRKYKGQVVAVDTYCWLHKGAIACAEKLAKGEPTDRYVGFCMKFVNMLLSHGIKPILVFDGCTLPSKKEVERSRRERRQANLLKGKQLLREGKVSEARECFTRSINITHAMAHKVIKAARSQGVDCLVAPYEADAQLAYLNKAGIVQAIITEDSDLLAFGCKKVILKMDQFGNGLEIDQARLGMCRQLGDVFTEEKFRYMCILSGCDYLSSLRGIGLAKACKVLRLANNPDIVKVIKKIGHYLKMNITVPEDYINGFIRANNTFLYQLVFDPIKRKLIPLNAYEDDVDPETLSYAGQYVDDSIALQIALGNKDINTFEQIDDYNPDTAMPAHSRSHSWDDKTCQKSANVSSIWHRNYSPRPESGTVSDAPQLKENPSTVGVERVISTKGLNLPRKSSIVKRPRSELSEDDLLSQYSLSFTKKTKKNSSEGNKSLSFSEVFVPDLVNGPTNKKSVSTPPRTRNKFATFLQRKNEESGAVVVPGTRSRFFCSSDSTDCVSNKVSIQPLDETAVTDKENNLHESEYGDQEGKRLVDTDVARNSSDDIPNNHIPGDHIPDKATVFTDEESYSFESSKFTRTISPPTLGTLRSCFSWSGGLGDFSRTPSPSPSTALQQFRRKSDSPTSLPENNMSDVSQLKSEESSDDESHPLREEACSSQSQESGEFSLQSSNASKLSQCSSKDSDSEESDCNIKLLDSQSDQTSKLRLSHFSKKDTPLRNKVPGLYKSSSADSLSTTKIKPLGPARASGLSKKPASIQKRKHHNAENKPGLQIKLNELWKNFGFKKDSEKLPPCKKPLSPVRDNIQLTPEAEEDIFNKPECGRVQRAIFQ

SEQ ID NO: 124 - protein, 413 aa, synthetic construct
CLAEGTRIFDPVTGTTHRIEDVVDGRKPIHVVAAAKDGTLLARPVVSWFDQGTRDVIGLRIAGGAIVWATPDHKVLTEYGWRAAGELRKGDRVAGPGGSGNSLALSLTADQMVSALLDAEPPILYSEYDPTSPFSEASMMGLLTNLADRELVHMINWAKRVPGFVDLTLHDQAHLLECAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRALDKITDTLIHLMAKAGLTLQQQHQRLAQLLLILSHIRHMSNKGMEHLYSMKYKNVVPLYDLLLEMLDAHRLHAGGSGASRVQAFADALDDKFLHDMLAEELRYSVIREVLPTRRARTFDLEVEELHTLVAEGVVVHNC

SEQ ID NO: 125 - protein, 413 aa, synthetic construct
CLAEGTRIFDPVTGTTHRIEDVVDGRKPIHVVAVAKDGTLLARPVVSWFDQGTRDVIGLRIAGGAIVWATPDHKVLTEYGWRAAGELRKGDRVAGPGGSGNSLALSLTADQMVSALLDAEPPILYSEYDPTSPFSEASMMGLLTNLADRELVHMINWAKRVPGFVDLTLHDQAHLLECAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRALDKITDTLIHLMAKAGLTLQQQHQRLAQLLLILSHIRHMSNKGMEHLYSMKYTNVVPLYDLLLEMLDAHRLHAGGSGASRVQAFADALDDKFLHDMLAEELRYSVIREVLPTRRARTFDLEVEELHTLVAEGVVVHNC

SEQ ID NO: 126 - protein, 413 aa, synthetic construct
CLAEGTRIFDPVTGTTHRIEDVVDGRKPIHVVAAAKDGTLLARPVVSWFDQGTRDVIGLRIAGGATVWATPDHKVLTEYGWRAAGELRKGDRVAGPGGSGNSLALSLTADQMVSALLDAEPPIPYSEYDPTSPFSEASMMGLLTNLADRELVHMINWAKRVPGFVDLTLHDQAHLLECAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRALDKITDTLIHLMAKAGLTLQQQHQRLAQLLLILSHIRHMSNKGMEHLYSMKYKNVVPLYDLLLEMLDAHRLHAGGSGASRVQAFADALDDKFLHDMLAEGLRYSVIREVLPTRRARTFDLEVEELHTLVAEGVVVHNC

SEQ ID NO: 127 - protein, 413 aa, synthetic construct
CLAEGTRIFDPVTGTTHRIEDVVDGRKPIHVVAAAKDGTLLARPVVSWFDQGTRDVIGLRIAGGATVWATPDHKVLTEYGWRAAGELRKGDRVAGPGGSGNSLALSLTADQMVSALLDAEPPILYSEYDPTSPFSEASMMGLLTNLADRELVHMINWAKRVPGFVDLTLHDQAHLLECAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRALDKITDTLIHLMAKAGLTLQQQHQRLAQLLLILSHIRHMSNKGMEHLYSMKYKNVVPLYDLLLEMLDAHRLHAGGSGASRVQAFADALDDKFLHDMLAEELRYSVIREVLPTRRARTFDLEVEELHTLVAEGVVVHNC

SEQ ID NO: 128 - protein, 413 aa, synthetic construct
CLAEGTRIFDPVTGTTHRIEDVVDGRKPIHVVAAAKDGTLLARPVVSWFDQGTRDVIGLRIAGGATVWATPDHKVLTEYGWRAAGELRKGDRVAGPGGSGNSLALSLTADQMVSALLDAEPPIPYSEYDPTSPFSEASMMGLLTNLADRELVHMINWAKRVPGFVDLTLHDQAHLLECAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRALDKITDTLIHLMAKAGLTLQQQHQRLAQLLLILSHIRHMSNKGMEHLYSMKYKNVVPLYDLLLEMLDAHRLHAGGSGASRVQAFADALDDKFLHDMLAEELRYSVIREVLPTRRARTFDLEVEELHTLVAEGVVVHNC

SEQ ID NO: 129 - protein, 413 aa, synthetic construct
CLAEGTRIFDPVTGTTHRIEDVVDGRKPIHVVAAAKDGTLLARPVVSWFDQGTRDVIGLRIAGGATVWATPDHKVLTEYGWRAAGELRKGDRVAGPGGSGNSLALSLTADQMVSALLDAEPPILYSEYNPTSPFSEASMMGLLTNLADRELVHMINWAKRVPGFVDLTLHDQAHLLERAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRALDKITDTLIHLMAKAGLTLQQQHQRLAQLLLILSHIRHMSNKGMEHLYSMKYKNVVPLYDLLLEMLDAHRLHAGGSGASRVQAFADALDDKFLHDMLAEGLRYSVIREVLPTRRARTFDLEVEELHTLVAEGVVVHNC

SEQ ID NO: 130 - protein, 413 aa, synthetic construct
CLAEGTRIFDPVTGTTHRIEDVVDGRKPIHVVAAAKDGTLLARPVVSWFDQGTRDVIGLRIAGGAIVWATPDHKVLTEYGWRAAGELRKGDRVAGPGGSGNSLALSLTADQMVSALLDAEPPILYSEYDPTSPFSEASMMGLLTNLADRELVHMINWAKRVPGFVDLTLHDQAHLLERAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRALDKITDTLIHLMAKAGLTLQQQHQRLAQLLLILSHIRHMSNKGMEHLYSMKYKNVVPLYDLLLEMLDAHRLHAGGSGASRVQAFADALDDKFLHDMLAEGLRYSVIREVLPTRRARTFDLEVEELHTLVAEGVVVHNC

SEQ ID NO: 131 - protein, 413 aa, synthetic construct
CLAEGTRIFDPVTGTTHRIEDVVDGRKPIHVVAVAKDGTLLARPVVSWFDQGTRDVIGLRIAGGATVWATPDHKVLTEYGWRAAGELRKGDRVAGPGGSGNSLALSLTADQMVSALLDAEPPILYSEYDPTSPFSEASMMGLLTNLADRELVHMINWAKRVPGFVDLTLHDQAHLLERAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRALDKITDTLIHLMAKAGLTLQQQHQRLAQLLLILSHIRHMSNKGMEHLYSMKYKNVVPLYDLLLEMLDAHRLHAGGSGASRVQAFADALDDKFLHDMLAEELRYSVIREVLPTRRARTFDLEVEELHTLVAEGVVVHNC

SEQ ID NO: 132 - skipped

SEQ ID NO: 133 - protein, 345 aa, Homo sapiens
MPARALLPRRMGHRTLASTPALWASIPCPRSELRLDLVLPSGQSFRWREQSPAHWSGVLADQVWTLTQTEEQLHCTVYRGDKSQASRPTPDELEAVRKYFQLDVTLAQLYHHWGSVDSHFQEVAQKFQGVRLLRQDPIECLFSFICSSNNNIARITGMVERLCQAFGPRLIQLDDVTYHGFPSLQALAGPEVEAHLRKLGLGYRARYVSASARAILEEQGGLAWLQQLRESSYEEAHKALCILPGVGTKVADCICLMALDKPQAVPVDVHMWHIAQRDYSWHPTTSQAKGPSPQTNKELGNFFRSLWGPYAGWAQAVLFSADLRQSRHAQEPPAKRRKGSKGPEG

SEQ ID NO: 134 - protein, 298 aa, Homo sapiens
MVTPALQMKKPKQFCRRMGQKKQRPARAGQPHSSSDAAQAPAEQPHSSSDAAQAPCPRERCLGPPTTPGPYRSIYFSSPKGHLTRLGLEFFDQPAVPLARAFLGQVLVRRLPNGTELRGRIVETEAYLGPEDEAAHSRGGRQTPRNRGMFMKPGTLYVYIIYGMYFCMNISSQGDGACVLLRALEPLEGLETMRQLRSTLRKGTASRVLKDRELCSGPSKLCQALAINKSFDQRDLAQDEAVWLERGPLEPSEPAVVAAARVGVGHAGEWARKPLRFYVRGSPWVSVVDRVAEQDTQA

SEQ ID NO: 135 - protein, 580 aa, Homo sapiens
MGTTGLESLSLGDRGAAPTVTSSERLVPDPPNDLRKEDVAMELERVGEDEEQMMIKRSSECNPLLQEPIASAQFGATAGTECRKSVPCGWERVVKQRLFGKTAGRFDVYFISPQGLKFRSKSSLANYLHKNGETSLKPEDFDFTVLSKRGIKSRYKDCSMAALTSHLQNQSNNSNWNLRTRSKCKKDVFMPPSSSSELQESRGLSNFTSTHLLLKEDEGVDDVNFRKVRKPKGKVTILKGIPIKKTKKGCRKSCSGFVQSDSKRESVCNKADAESEPVAQKSQLDRTVCISDAGACGETLSVTSEENSLVKKKERSLSSGSNFCSEQKTSGIINKFCSAKDSEHNEKYEDTFLESEEIGTKVEVVERKEHLHTDILKRGSEMDNNCSPTRKDFTGEKIFQEDTIPRTQIERRKTSLYFSSKYNKEALSPPRRKAFKKWTPPRSPFNLVQETLFHDPWKLLIATIFLNRTSGKMAIPVLWKFLEKYPSAEVARTADWRDVSELLKPLGLYDLRAKTIVKFSDEYLTKQWKYPIELHGIGKYGNDSYRIFCVNEWKQVHPEDHKLNKYHDWLWENHEKLSLS

SEQ ID NO: 136 - protein, 410 aa, Homo sapiens
MEAENAGSYSLQQAQAFYTFPFQQLMAEAPNMAVVNEQQMPEEVPAPAPAQEPVQEAPKGRKRKPRTTEPKQPVEPKKPVESKKSGKSAKSKEKQEKITDTFKVKRKVDRFNGVSEAELLTKTLPDILTFNLDIVIIGINPGLMAAYKGHHYPGPGNHFWKCLFMSGLSEVQLNHMDDHTLPGKYGIGFTNMVERTTPGSKDLSSKEFREGGRILVQKLQKYQPRIAVFNGKCIYEIFSKEVFGVKVKNLEFGLQPHKIPDTETLCYVMPSSSARCAQFPRAQDKVHYYIKLKDLRDQLKGIERNMDVQEVQYTFDLQLAQEDAKKMAVKEEKYDPGYEAAYGGAYGENPCSSEPCGFSSNGLIESVELRGESAFSGIPNGQWMTQSFTDQIPSFSNHCGTQEQEEESHA

SEQ ID NO: 137 - DNA, 125 bp, synthetic construct
  source 1..125: mol_type = other DNA
nnnnnnnngtttttgtactctcaagatttagaaataaatcttgcagaagctacaaagataaggcttcatgccgaaatcaacaccctgtcattttatggcagggtgttttcgttatttaatttttt

SEQ ID NO: 138 - DNA, 121 bp, synthetic construct
  source 1..121: mol_type = other DNA
nnnnnnnnnnnnnnnnnngtttttgtactctcagaaatgcagaagctacaaagataaggcttcatgccgaaatcaacaccctgtcattttatggcagggtgttttcgttatttaatttttt

SEQ ID NO: 139 - DNA, 109 bp, synthetic construct
  source 1..109: mol_type = other DNA
nnnnnnnnnnnnnnnnnnnngtttttgtactctcagaaatgcagaagctacaaagataaggcttcatgccgaaatcaacaccctgtcattttatggcagggtgtttttt

SEQ ID NO: 140 - DNA, 102 bp, synthetic construct
  source 1..102: mol_type = other DNA
nnnnnnnnnnnnnnnnnnnngttttagagctagaaatagcaagttaaaataaggctagtccgttatcaacttgaaaaagtggcaccgagtcggtgctttttt

SEQ ID NO: 141 - DNA, 88 bp, synthetic construct
  source 1..88: mol_type = other DNA
nnnnnnnnnnnnnnnnnnnngttttagagctagaaatagcaagttaaaataaggctagtccgttatcaacttgaaaaagtgttttttt

SEQ ID NO: 142 - DNA, 76 bp, synthetic construct
  source 1..76: mol_type = other DNA
nnnnnnnnnnnnnnnnnnnngttttagagctagaaatagcaagttaaaataaggctagtccgttatcatttttttt

SEQ ID NO: 143 - RNA, 82 nt, synthetic construct
  source 1..82: mol_type = other RNA
gttttagagctagaaatagcaagttaaaataaaggctagtccgttatcaacttgaaaaagtggcaccgagtcggtgcttttt

SEQ ID NO: 144 - DNA, 33 bp, synthetic construct
  source 1..33: mol_type = other DNA
gccaacatgaggatcacccatgtctgcagggcc

SEQ ID NO: 145 - protein, 131 aa, synthetic construct
GSASNFTQFVLVDNGGTGDVTVAPSNFANGVAEWISSNSRSQAYKVTCSVRQSSAQNRKYTIKVEVPKVATQTVGGEELPVAGWRSYLNMELTIPIFATNSDCELIVKAMQGLLKDGNPIPSAIAANSGIY

SEQ ID NO: 146 - DNA, 10 bp, synthetic construct
  source 1..10: mol_type = other DNA
gttttaaaac

SEQ ID NO: 147 - DNA, 905 bp, synthetic construct
  source 1..905: mol_type = other DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgttcactgccgtataggcagggcccagactgagcacgtgagttttagagctagaaatagcaagttaaaataaggctagtccgttatcaacttgaaaaagtgggaccgagtcggtcctctgccatcaaagcgtgctcagtctgttttagggtcatgaaggtttttcttttcctgagaaaacaacacgtattgttttctcaggttttgctttttggcctttttctagcttaaaaaaaaaaaaagcaaaagatgctggtggttggcactcctggtttccaggacggggttcaaatccctgcggcgtctttgctttgact

SEQ ID NO: 148 - DNA, 942 bp, synthetic construct
  source 1..942: mol_type = other DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgttcactgccgtataggcagggcccagactgagcacgtgagttttagagctagaaatagcaagttaaaataaggctagtccgttatcaacttgaaaaagtgggaccgagtcggtcctctgccatcaaagcgtgctcagtctgttttgttttggctgggtttttccttgttcgcaccggacacctccagtgaccagacggcaaggtttttatcccagtgtatattggaaaaacatgttatacttttgacaatttaacgtgcctagagctcaaattaaactaataccataacgtaatgcaacttacaacataaataaaggtcaatgtttaatccataaaaaaaaaaaaaaaaaaa

SEQ ID NO: 149 - DNA, 1370 bp, synthetic construct
  source 1..1370: mol_type = other DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgttcactgccgtataggcagggcccagactgagcacgtgagttttagagctagaaatagcaagttaaaataaggctagtccgttatcaacttgaaaaagtgggaccgagtcggtcctctgccatcaaagcgtgctcagtctgttttgttttggctgggtttttccttgttcgcaccggacacctccagtgaccagacggcaaggtttttatcccagtgtatattggaaaaacatgttatacttttgacaatttaacgtgcctagagctcaaattaaactaataccataacgtaatgcaacttacaacataaataaaggtcaatgtttaatccataaaaaaaaaaaaaaaaaaaacacactgttttggctgggtttttccttgttcgcaccggacacctccagtgaccagacggcaaggtttttatcccagtgtatattggaaaaacatgttatacttttgacaatttaacgtgcctagagctcaaattaaactaataccataacgtaatgcaacttacaacataaataaaggtcaatgtttaatccataaaaaaaaaaaaaaaaaaatctctctgttttggctgggtttttccttgttcgcaccggacacctccagtgaccagacggcaaggtttttatcccagtgtatattggaaaaacatgttatacttttgacaatttaacgtgcctagagctcaaattaaactaataccataacgtaatgcaacttacaacataaataaaggtcaatgtttaatccataaaaaaaaaaaaaaaaaaa

SEQ ID NO: 150 - DNA, 807 bp, synthetic construct
  source 1..807: mol_type = other DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgttcactgccgtataggcagggcccagactgagcacgtgagttttagagctagaaatagcaagttaaaataaggctagtccgttatcaacttgaaaaagtgggaccgagtcggtcctctgccatcaaagcgtgctcagtctgtttgtttcaaaagtagactgtacgctaagggtcatatctttttttgtttggtttgtgtcttggttggcgtcttaaa

SEQ ID NO: 151 - DNA, 743 bp, synthetic construct
  source 1..743: mol_type = other DNA
ctaaggaccagcttctttgggagagaacagacgcaggggcgggagggaaaaagggagaggcagacgtcacttccccttggcggctctggcagcagattggtcggttgagtggcagaaaggcagacggggactgggcaaggcactgtcggtgacatcacggacagggcgacttctatgtagatgaggcagcgcagaggctgctgcttcgccacttgctgcttcaccacgaaggagttcccgtgccctgggagcgggttcaggaccgctgatcggaagtgagaatcccagctgtgtgtcagggctggaaagggctcgggagtgcgcggggcaagtgaccgtgtgtgtaaagagtgaggcgtatgaggctgtgtcggggcagaggcccaagatctcagttcactgccgtataggcagggcccagactgagcacgtgagttttagagctagaaatagcaagttaaaataaggctagtccgttatcaacttgaaaaagtgggaccgagtcggtcctctgccatcaaagcgtgctcagtctgtttcagcaagttcagagaaatctgaacttgctggatttttggagcagggagatggaataggagcttgctccgtccactccacgcatcgacctggtattgcagtacctccaggaacggtgcacccactttctggagtttcaaaagtagactgtacgctaagggtcatatctttttttgtttggtttgtgtcttggttggcgtcttaaa

SEQ ID NO: 152 - DNA, 141 bp, synthetic construct
  source 1..141: mol_type = other DNA
ggcccagactgagcacgtgagttttagagctagctcatgaaaatgagctagcaagttaaaataaggctagtccgttatcaacttgaaaaagtgggaccgagtcggtcctctgccatcaaagcgtgctcagtctgttttttt

SEQ ID NO: 153 - DNA, 129 bp, synthetic construct
  source 1..129: mol_type = other DNA
ggcccagactgagcacgtgagtttgagagctagaaatagcaagtttaaataaggctagtccgttatcaacttgaaaaagtgggaccgagtcggtcctctgccatcaaagcgtgctcagtctgttttttt

SEQ ID NO: 154 - DNA, 197 bp, synthetic construct
  source 1..197: mol_type = other DNA
ggcccagactgagcacgtgagttttagagctagaaatagcaagttaaaataaggctagtccgttatcaacttgaaaaagtgggaccgagtcggtcctctgccatcaaagcgtgctcagtctgggccggcatggtcccagcctcctcgctggcgccggctgggcaacatgcttcggcatggcgaatgggacttttttt

SEQ ID NO: 155 - DNA, 171 bp, synthetic construct
  source 1..171: mol_type = other DNA
ggtgggagacgtcccaccggcccagactgagcacgtgagttttagagctagaaatagcaagttaaaataaggctagtccgttatcaacttgaaaaagtgggaccgagtcggtcctctgccatcaaagcttcgaccgtgctcagtctggtgggagacgtcccaccttttttt

SEQ ID NO: 156 - DNA, 180 bp, synthetic construct
  source 1..180: mol_type = other DNA
gagcagcatggcgtcgctgctcacggcccagactgagcacgtgagttttagagctagaaatagcaagttaaaataaggctagtccgttatcaacttgaaaaagtgggaccgagtcggtcctctgccatcaaagcttcgaccgtgctcagtctccatcagttgacaccctgaggttttttt

SEQ ID NO: 157 - DNA, 188 bp, synthetic construct
  source 1..188: mol_type = other DNA
  modified_base 15: mod_base = OTHER; note = uracil
  modified_base 61: mod_base = OTHER; note = uracil
  modified_base 101: mod_base = OTHER; note = uracil
gcagacctaagtggtgacatatggtctgggcccagactgagcacgtgagttttagagctatacgtagcaagttaaaataaggctagtccgttatcaactttacgaagtgggaccgagtcggtcctctgccatcaaagcttcgaccgtgctcagtctgcatgcgattagaaataatcgcatgttttttt

SEQ ID NO: 158 - DNA, 125 bp, synthetic construct
  source 1..125: mol_type = other DNA
tctgccatcaaagctgcgaccgtgctcagtctggtgggagacgtcccaccggccggcatggtcccagcctcctcgctggcgccggctgggcaacatgcttcggcatggcgaatgggacttttttt

SEQ ID NO: 159 - protein, 400 aa, synthetic construct
EANQGAPGIDGVSTDQLRDYIRAHWSTIHAQLLAGTYRPAPVRRVEIPKPGGGTRQLGIPTVVDRLIQQAILQELTPIFDPDFSSSSFGFRPGRNAHDAVRQAQGYIQEGYRYVVDMDLEKFFDRVNHDILMSRVARKVKDKRVLKLIRAYLQAGVMIEGVKVQTEEGTPQGGPLSPLLANILLDVLDKELEKRGLKFCRYADDCNIYVKSLRAGQRVKQSIQRFLEKTLKLKVNEEKSAVDRPWKRAFLGFSFTPERKARIRLAPRSIQRLKQRIRQLTNPNWSISMPERIHRVNQYVMGWIGYFRLVETPSVLQTIEGWIRRRLRLCQWLQWKRVRTRIRELRALGLKETAVMEIANTRKGAWRTTKTPQLHQALGKTYWTAQGLKSLTQRYFELRQG

SEQ ID NO: 160 - protein, 419 aa, synthetic construct
ALLERILARDDLITALKRVEANQGAPGIDGVSTDQRRDYIRAHWSTIHAQLLAGTYRPAPVRRVEIPKPGGGTRQLVIPTVVDRLIQQAILQELTPIFDPDFSSSSFGFRPGRNAHDAVRQAQGYIQEGYRYVVDMDLEKFFDRVNHDILMSRVARKVKDKRVLKLIRAYLQAGVMIEGVKVQTEEGTPQGGPLSPLLANILLDDLDKELEKRGLKFCRYADDCNIYVKSLRAGQRVKQSIQRFLEKTLKLKVNEEKSAVDRPWKRAFLGFSFTPERKARIRLAPRSIQRLKQRIRQLTNPNWSISMPERIHRVNQYVMGWIGYFRLVETPSVLQTIEGWIRRRLRLCQWLQWKRVRTRIRELRALGLKETAVMEIANTRKGAWRTTKTPQLHQALGKTYWTAQGLKSLTQRYFELRQG

SEQ ID NO: 161 - protein, 419 aa, synthetic construct
ALLERILARDNLITELKRVEANQGAPGIDGVSTDQPRDYIRAHWSTIHAQLLAGTYRPAPVRRVEIPKPGGGTRQLGIPTVVDRLIQQAILQELTPIFDPDFSSSSFGFRPGRNAHDAVRQVQGYIQEGYRYVVDMDLEKFFDRVNHDILMSRVARKVKDKRVLKLIRAYLQAGVMIEGVKVQTEEGTPQGGPLSPLLANILLDDLDKELEKRGLKFCRYADDCNIYVKSLRAGQRVKQSIQRFLEKTLKLKVNEEKSAVDRPWKRAFLGFSFTPERKARIRLAPRSIQRLKQRIRQLTNPNWSISMPERIHRVNQYVMGWIGYFRLVETPSVLQTIEGWIRRRLRLCQWLQWKRVRTRIRELRALGLKETAVMEIANTRKGAWRTTKTPQLHQALGKTYWTAQGLKSLTQRYFELRQG

SEQ ID NO: 162 - protein, 419 aa, synthetic construct
ALLERILARDNLITVLKRVEANQGAPGIDGVSTDQLHDYIRAHRSTIHAQLLAGTYRPAPVRRVEIPKPGGGTRQLGIPTVVDRLIQQAILQELTPIFDPDFSSSSFGFRPGRNAHDAVRQAQGFIQEGYRYVVDMDLEKFFDRVNHDILMSRVARKVKDKRVLKLIRAYLQAGVMIEGVKVQTEEGTPQGGPLSPLLANILLDDLDKELEKRGLKFCRYADDCNIYVKSLRAGQRVKQSIQRFLEKTLKLKVNEEKSAVDRPWKRAFLGFSFTPERKARIRLAPRSIQRLKQRIRQLTNPNWSISMPERIHRVNQYVMGWIGYFRLVETPSVLQTIEGWIRRRLRLCQWLQWKRVRTRIRELRALGLKETAVMEIANTRKGAWRTTKTPQLHQALGKTYWTAQGLKSLTHRYFELRQG

SEQ ID NO: 163 - protein, 419 aa, synthetic construct
ALLERILARDNLITVLKRVEANQGAPGIDGVSTDQLHDYIRAHRSTIHAQLLAGTYRPAPVRRVEIPKPGGGTRQLGIPTVVDRLIQQAILQELTPIFDPDFSSSSFGFRPGRNAHDAVRQAQGYIQEGYRYVVDMDLEKFFDRVNHDILMSRVARKVKDKRVLKLIRAYLQAGVMIEGVKVQTEEGTPQGGPLSPLLANILLDDLDKELEKRGLKFCRYADDCNIYVKSLRAGQRVKQSIQRFLEKTLKLKVNEEKSAVDRPWKRAFLGFSFTPERKARIRLAPRSIQKLKQRIRQLTNPNWSISMPERIHRVNQYVMGWIGYFRLVETPSVLQTIEGWIRRRLRLCQWLQWKRVRTRIRELRALGLKETAVMEIANTRKGAWRTTKTPQLHQALGKTYWTAQGLKSLTQRYFELRQG

SEQ ID NO: 164 - protein, 419 aa, synthetic construct
ALLERILARDDLITALKRVEANQGAPGIDGVSTDQRRDYIRAHWSTIHAQLLAGTYRPAPVRRVEIPKPGEGTRQLGIPTVVDRLIQQAILQELTPIFDPDFSSSSFGFRPGRNAHDAVRQAQGYIQGGYRYVVDMDLEKFFDRVNHDILMSRVARKVKDKRVLKLIRAYLQAGVMIEGVKVQTEEGTPQGGPLSPLLANILLDDLDKELEKRGLKFCRYADDCNIYVKSLRAGQRVKQSIQRFLEKTLKLKVNEEKSAVDRSWKRAFLGFSFTPERKARIRLAPRSIQRLKQRIRQLTNPNWSISMPERIHRVNQYVMGWIGYFRLVETPSVLQTIEGWIRSRLRLCQWLQWKRVRTSIRELRALGLKETAVMEIANTRKGAWRTTKTPQLHQALGKTYWTAQGLKSLTQRYFELRQG

SEQ ID NO: 165 - protein, 419 aa, synthetic construct
ALLERILARDDLITALKRVEANQGAPGIDGVSTDQLRDCIRAHWSTIHAQLLAGTYRPAPVRRVETPKPGGVTRQLGIPTVVDRLIQQAILRELTPIFDPDFSSSSFGFRPGRNAHDAVRQAQGYIQEGYRYVVDMDLEKFFDRVNHDILMSRVARKVKDKRVLKLIRAYLQAGVMIEGVKVQTEEGTPQGGPLSPLLANILLDDLDKELEKRGLKFCRYADDCNIYVKSLRAGQRVKQSIQRFLEKTLKLKVNEEKSAVDRPWKRAFLGFSFTPERKARIRLAPISIQRLKQRIRQLTNPNWSISMPERIHRVNQYVMGWIGYFRLVETPSVLQTIEGWIRRRLRLCQWLQWKRVSTRIRELRALGLKETAVMEIANTRKGAWRTTKTPQLHQALGKTYWTAQGLKSLTQRYFELRQG

SEQ ID NO: 166 - protein, 419 aa, synthetic construct
ALLERILARDDLITALKRVEANQGAPGIDGVSTDQLRDCIRAHWSTIHAQLLAGTYRPAPVRRVETPKPGGVTRQLGIPTVVDRLIQQAILRELTPIFDPDFSSSSFGFRPGRNAHDAVRQAQGYIQEGYRYVVDMDLEKFFDRVNHDILMSRVARKVKDKRVLKLIRAYLQAGVMIEGVKVQTEEGTPQGGPLSPLLANILLDDLDKELEKRGLKFCRYADDCNIYVKSLRAGQRVKQSIQRFLEKTLKLKVNEEKSAVDRPWKRAFLGFSFTPERKARIRLAPRSIQRLKQRIRQLTNPNWSISMPERIHRVNQYVMGWIGYFRLVETPSVLQTIEGWIRRRLRLCQWLQWKRVSTRIRELRALGLKETAVMEIANTRKGAWRTTKTPQLHQALGKTYWTAQGLKSLTQRYFELRQG

SEQ ID NO: 167 - protein, 419 aa, synthetic construct
ALLERILARDDLITALKRVEANQGAPGIDGVSTDQLRDYNRAHWSTIHAQLLAGTYRPAPVRRVEIPKPGGGTRQLGIPTVVDRLIQQAILQELTPIFDPDFSSSSFGFRPGRNAHDAVRQAQGYIQEGYRYVVDMDLEKFFDRVNHDILMSRVARKVKDKRVLKLIRAYLQAGVMIEGVKVQTEEGTLQGGPLSPLLANILLDDLDKELEKRGLKFCRYADDCNIYVKSLRVGQRVKQSIQRFLEKTLKLKVNEEKSAVDRPWKRAFLGFSFTPEREARIRLAPRSIQRLKQRIRQLTNPNWSISMPERIHRVNQYVMGWIGYFRLVETPSVLQTIEGWIRRRLRLCQWLQWKRVRTRIRELRALGLKETAVMEIANTRKGAWRTTKTPQLHQALGKTYWTAQGLKSLTQRYFELRQG

SEQ ID NO: 168 - protein, 419 aa, synthetic construct
ALLERILARDDLITALKRVEANQGAPGIDGVSTDQRRDYIRAHWSTIHAQLLAGTYRPAPVRRVEIPKPGGGTRQLGIPTVVDRLIQQAILQELTPIFDPDFSSSSFGFRPGRNAHDAVRQAQGYIQEGYRYVVDMDLEKFFDRVNHDILMSRVARKVKDKRVLKLIRAYLQAGVMIEGVKVQTEEGTPQGGPLSPLLANILLDDLDKELEKRGLKFCRYADDCNIYVKSLRAGQRVKQSIQRFLEKTLKLKVNEEKSAVDRPWKMAFLGFSFTPERKARIRLAPRSIQRLKQRIRQLTNPNWSISMTERIHRVNQYVMGWIGYFRLVETPSVLQTIEGWIRRRLRLCQWLQWKRVSTRIRGLRALGLKETAVMEIANTRKGAWRTTKTPQLHQALGKTYWTAQGLKSLTQRYFELRQG

SEQ ID NO: 169 - protein, 419 aa, synthetic construct
ALLERILARDNLITVLKRGEANQGAPGIDGVSTDQLRDYSRAHWSTIHAQLLAGTYRPAPVRRVEIPKPGGGTRQLGIPTVVDRLIQQAILQELTPIFDPDFSSSSFGFRPGRNAHDAVRQAQGYIQEGYRYVVDMDLEKFFDRVNHDILMSRVARKVKDKRVLKLIRAYLQAGVMIEGVKVQTEEGTPQGGPLSPLLANILLDDLDKELEKRGLKFCRYADDCNIYVKSLKAGQRVKQSIQRFLEKTLKLKVNEEKSAVDRSWKRAFLGFSFTPERKARIRLAPRSIQRLKQRIRQLTNPNWSISMPERIHRVNQYVMGWIGYFRLVETPSVLQTIEGWIRRRLRLCQWLQWKRVRTRIRELRALGLKETAVMEIANTRKGAWRTTKTPQLHQALGKTYWTAQGLKSLTQRYFELRQG

SEQ ID NO: 170 - protein, 419 aa, synthetic construct
ALLERILARDNLITAPKRGEANQGAPGIDGVSTDQLRDYSRAHWSTIHAQLLAGTYRPAPVRRVERPKPGGGTRQLGIPTVVDRLIQQAILQELTPIFDPDFSSSSFGFRPGRNAHDAVRQAQGYIQEGYRYVVDMDLEKFFDRVNHDILMSRVARKVKDKRVLKLIRAYLQAGVMIEGVKVQTEEGTPQGGPLSPLLANILLDDLDKELEKRGLKFCRYADDCNIYVKSLRAGQRVKQSIQRFLEKTLKLKVNEEKSAVDGSWKRAFLGFSFTPERKARIRLAPRSIQRLKQRIRQLTNPNWSISMPERIHRVNQYVMGWIGYFRLVETPSVLQTIEGWIRRRLRLCQWLQWKRVRTRIRELRALGLKETAAMEIANTRKGAWRTTKTPQLHQALGKTYWTAQGLKSLTQRYFELRQG

SEQ ID NO: 171 - protein, 419 aa, synthetic construct
ALLERILARDNLITAPKRGEANQGAPGIDGVSTDQLRDYSRAHWSTIHAQLLAGTYRPAPVRRVERPKPGGGTRQLGIPTVVDRLIQQAILQELTPIFDPDFSSSSFGFRPGRNAHDAVRQAQGYIQEGYRYVVDMDLEKFFDRVNHDILMSRVARKVKDKNVLKLIRAYLQAGVMIEGVKVQTEEGTPQGGPLSPLLANILLDDLDKELEKRGLKFCRYADDCNIYVKSLRAGQRVKQSIQRFLEKTLKLKVNEEKSAVDGSWKRAFLGFSFTPERKARIRLAPRSIQRLKQRIRQLTNPNWSISMPERIHRVNQYVMGWIGYFRLVETPSVLQTIEGWIRRRLRLCQWLQWKRVRTRIRELRALGLKETAVMEIANTRKGAWRTTKTPQLHQALGKTYWTAQGLKSLTQRYFELRQG

SEQ ID NO: 172 - protein, 497 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFSEALHRDLADFRIQHPDLILLQYADDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQKIKQALLTAPALGLPDLTKPFELFVDEKQGYAKGVLTQKLGPWCRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLD

SEQ ID NO: 173 - protein, 497 aa, synthetic construct
TLNIEDEYRLHETSKEPDVSLGSTWLSDFPQAWAETGGMGLAVRQAPLIIPLKATSTPVYIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYADDLLLAATSELDCQQGTRALLQTLGSLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLD

SEQ ID NO: 174 - protein, 497 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRLVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYADDLLLAATSELDCQQGTRALLQTLGNLGYRASAKKAQICQKQVKYLGYLLKEGQRWLTEARKEAVMGQPTPKTPRQLREFLGKAGFCRLFIPRFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLD

SEQ ID NO: 175 - protein, 497 aa, synthetic construct
TLNIEDEYRLHETSKEPDVSLGSTWLSDFPQAWAETGGMGLAVRQAPLIIPLKATSTPVYIKQYPMSQEARLGIKPHIQRLLDQGILVPCQSPWNTPLLPVKKPGTNDYRPVQDLREVNKRVEDIHPTVPNPYNLLSRLPPSHQWYTVLDLKDAFFCLRLHPTSQPLFAFEWRDPEMGISGQLTWTRLPQGFKNSPTLFNEALHRDLADFRIQHPDLILLQYADDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLD

SEQ ID NO: 176 - protein, 497 aa, synthetic construct
TLNIEDEYRLHETSKEPDVSLGSTWLSDFPQAWAETGGMGLAVRQAPLIIPLKATSTPVYIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQFADDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVNQPPDRWLSNARMTHYQALLLDTDRVQFGPVVALNPATLLPLPEEGLQHNCLD

SEQ ID NO: 177 - protein, 497 aa, synthetic construct
TLNIEDEYRLHETSKEPDVSLGSTWLSDFPQAWAETGGMGLAVRQAPLIIPLKATSTPVYIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFFLRLHPTSQPLFAFEWRDPEMGISGQLTWTRLPQGFKNSPTLFNEALHRDLADFRIQHPDLILLQYADDLLLAATSELDCQQGTRALLQILGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLD

SEQ ID NO: 178 - protein, 497 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYMDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQSLLLDTDRVQFGPVVALNPATLLPLPEEGLQHNCLD

SEQ ID NO: 179 - protein, 497 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRKFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLD

SEQ ID NO: 180-187 - skipped (8 entries)

SEQ ID NO: 188 - protein, 407 aa, synthetic construct
GRPYVTLNLNGMFMDKFKPYSKSNAPITTLEKLSKALSISVEELKAIAELSLDEKYTLKEIPKIDGSKRIVYSLHPKMRLLQSRINERIFKELVVFPSFLFGSVPSKNDVLNSNVKRDYVSCAKAHCGAKTVLKVDISNFFDNIHRDLVRSVFEEILHIKDEALEYLVDICTKDDFVVQGALTSSYIATLCLFAVEGDVVRRAQKKGLVYTRLLDDITVSSKISNYDFSQMQSHIERMLSEHNLPINKHKTKIFHCSSEPIKVHGLIVDYDSPRLPSDEVKRIRASIHNLKLLAAKNNTKTSVAYRKEFNRCMGRVSELGRVGQEEYESFKKQLQAIKPMPSKRDVAVIDAAIKSLELSYSKGNQNKHWYKRKYDLTRYKMIILTRSESFKEKLECFKSRLASLKPL

SEQ ID NO: 189 - protein, 407 aa, synthetic construct
GRPYVTLNLNGMFMDKFKPYSKSNAPITTLEKLSKALSISVEELKAIAELSLDKKYTLKEIPKIDGSKRIVYSLHPKMRLLQSRINERIFKELVVFPSFLFGSVPSKNDVLNSNVKRDYVSCAKAHCGAKTVLKVDISNFFDNIHRDLVRSVFEEILHIKDEALEYLVDICTKDDFVVQGALTSSYIATLCLFAVEGDVVRRAQRKGLVYTRLVDDITVSSKISNYDFSQMQSHIERMLSEHNLPINKHKTKIFHCSSEPIKVHGLIVDYDSPRLPSDKVKRIRASIHNLKLLAAKNNTKTSVAYRKEFNRCMGRVNELGRVGHEKYESFKKQLQAIKPMPSKRDVAVIDAAIKSLELSYSKGNQNKHWYKRKYDLTRYKMIILTRSESFKEKLECFKSRLASLKPL

SEQ ID NO: 190 - protein, 407 aa, synthetic construct
GRPYVTLNLNGMFMDKFKPYSKSNAPITTLEKLSKVLSISVEELKAIAELSLDEKYTLKEIPKIDGSKRIVYSLHPKMRLLQSRINERIFKELVVFPSFLFGSVPSKNDVLNSNVKRDYVSCAKAHCGAKTVLKVDISNFFDNIHRDLVRSVFEEILHIKDEALEYLVDICTKDDFVVQGALTSSYIATLCLFAVEGDVVRRAQKKGLVYTRLVDDITVSSKISNYDFSQMQSHIERMLSEHNLPINKHKTKIFHCSSEPIKVHGLIVDYDSPRLPSDKVKRIRASIHNLKLLAAKNNTKTSVAYRKEFNRCMGRVNELGRVGHEKYESFKKQLQAIKPMPSKRDVAVIDAAIKSLELSYSKGNQNKHWYKRKYDLTRYKMIILTRSESFKEKLECFKSRLASLKPL

SEQ ID NO: 191 - protein, 407 aa, synthetic construct
GRPYVTLNLNGMFMDKFKPYSKSNAPITTLEKLSKALSISVEELKAIAELSLDKKYTLKEIPKIDGSKRIVYSLHPKMRLLQSRINERIFKELVVFPSFLFGSVPSKNDVLNSNVKRDYVSCAKAHCGAKTVLKVDISNFFDNIHRDLVRSVFEEILHIKDEALEYLVDICTKDDFVVQGALTSSYIATLCLFAVEGDVVRRAQRKGLVYTRLVDDITVSSKISNYDFSQMQSHIERMLSEHNLPINKHKTKIFHCSSEPIKVHGLIVDYDSPRLPFDKVKRIRASIHNLKLLAAKNNTKTSVAYRKEFNRCMGRVNELGRVGHEKYESFKKQLQAIKPMPSKRDVAVIDAAIKSLELSYSKGNQNKHWYKRKYDLTRYKMIILTRSESFKEKLECFKSRLASLKPL

SEQ ID NO: 192 - protein, 407 aa, synthetic construct
GRPYVTLNLNGMFMDKFKPYSKSNAPITTLEKLSKALSISVEELKAIAELSLDEKYTLKKIPKIDGSKRIVYSLHPKMRLLQSRINERIFKELVVFPSFLFGSVPSKNDVLNSNVKRDYVSCAKAHCGAKTVLKVDISNFFDNIHRDLVRSVFEEILHIKDEALDYLVDICTKDDFVVQGALTSSYIATLCLFAVEGDVVRRAQRKGLVYTRLVDDITVSSKISNYDFSQMQSHIERMLSEHNLPINKHKTKIFHCSSEPIKVHGLIVDYDSPRLPSDKVKRIRASIHNLKLLAAKNNTKTSVAYRKEFNRCMGRVNELGRVGHEKYESFKKQLQAIKPMPSNRDVAVIDAAIKSLELSYSKGNQNKHWYKRKYDLTRYKMIILTRSESFKEKLECFKSRLASLKPL

SEQ ID NO: 193 - protein, 407 aa, synthetic construct
GRPYVTLNLNGMFMDKFKPYSKSNAPITTLEKLSKALSISVEELKAIAELSLDEKYTLKKIPKIDGSKRIVYSLHPKMRLLQSRINERIFKELVVFPSFLFGSVPSKNDVLNSNVKRDYVSCAKAHCGAKTVLKVDISNFFDNIHRDLVRTVFEEILHIKDEALDYLVDICTKDDFVVQGALTSSYIATLCLFAVEGDVVRRAQRKGLVYTRLVDDITVSSKISNYDFSQMQSHIERMLSEHNLPINKHKTKIFHCSSEPIKVHGLIVDYDSPRLPSDKVKRIRASIHNLKLLAAKNNTKTSMAYRKEFNRCMGRVNELGRVGHEKYESFKKQLQAIKPMPSNRDVAVIDAAIKSLELSYSKGNQNKHWYKRKYDLTRYKMIILTRSESFKEKLECFKSRLASLKPL

SEQ ID NO: 194 - protein, 407 aa, synthetic construct
GRPYVTLNLNGMFMDKFKPYSKSNAPITTLEKLSKALSISVEELKAIAELSLDEKYTLKEIPKIDGSKRIVYSLHPKMRLLQSRINKRIFKELVVFPSFLFGSVPSKNDVLNSNVKRDYVSCAKAHCGAKTVLKVDISNFFDNIHRDLVRSVFEEILHIKDEALEYLVDICTKDDFVVQGALTSSYIATLCLFAVEGDVVRRAQRKGLVYTRLVDDITVSSKISNYDFSQMQSHIERMLSEHDLPINKHKTKIFHCSSEPIKVHGLIVDYDSPRLPSDEVKRIRASIHNLKLLAAKNNTKTSVAYRKEFNRCMGRVNELGRVGHEEYKSFKKQLQAIKPMPSKRDVAVIDAAIKSLELSYSKGNQNKHWYKKKYDLTRYKMIILTRSESFKEKLECFKSRLASLKPL

SEQ ID NO: 195 - protein, 407 aa, synthetic construct
GRPYVTLNLNGMFMDKFKPYSKSNAPITTLEKLSKALSISVEELKAIAELSLDEKYTLKEIPKIDGSKRIVYSLHPKMRLLQSRINKRIFKELVVFPSFLFGSVPSKNDVLNSNVKRDYVSCAKAHCGAKTVLKVDISNFFDNIHRDLVRSVFEEILHIKDEALEYLVDICTKDDFVVQGANTSSYIANLCLFAVEGDVVRRAQRKGLVYTRLVDDITVSSKISNYDFSQMQSHIERMLSEHDLPINKHKTKIFHCSSEPIKVHGLRVDYDSPRLPSDEVKRIRASIHNLKLLAAKNNTKTSVAYRKEFNRCMGKVNKLGRVGHEKYESFKKQLQAIKPMPSKRDVAVIDAAIKSLELSYSKGNQNKHWYKRKYDLTRYKMIILTRSESFKEKLECFKSRLASLKPL

SEQ ID NO: 196 - protein, 505 aa, synthetic construct
ISSSKHTLSQMNKVSNIVKEPKLPDIYKEFKDITADTNTEKLPKPIKGLEFEVELTQENYRLPIRNYPLTPVKMQAMNDEINQGLKSGIIRESKAINACPVIFVPRKEGTLRMVVDYRPLNKYVKPNIYPLPLIEQLLTKIQGSTIFTKLDLKSAYHQIRVRKGDEHKLAFRCPRGVFEYLVMPYGISTAPAHFQYFINTILGEAKESHVVCYMDDILIHSKSESEHVKHVKDVLQKLKNANLIINQAKCEFHQSQVKFIGYHISEKGLTPCQENIDKVLQWKQPKNRKELRQFLGQVNYLRKFIPKTSQLTHPLNKLLKKDVRWKWTPTQTQAIENIKQCLVSPPVLRHFDFSKKILLETDVSDVAVGAVLSQKHDDDKYYPVGYYSAKMSKAQLNYSVSDKEMLAIIKSLEHWRHYLESTIEPFKILTDHRNLIGRITNESEPENKRLARWQLFLQDFNFEINYRPGSANHIADALSRIVDETEPIPKDNEDNSINFVNQISI

SEQ ID NO: 197 - protein, 505 aa, synthetic construct
ISSSKHTLSQMNKASNIVKEPELPDIYKEFKDITADTNTEKLPKPIKGLEFEVELTQENYRLPIRNYPLPPGKMQAMNDEINQGLKSGIIRESKAINACPVMFVPKKEGTLRMVVDYKPLNKYVKPNIYPLPLIEQLLAKIQGSTIFTKLDLKSAYHQIRVRKGDEHKLAFRCPRGVFEYLVMPYGISTAPAHFQYFINTILGEAKESHVVCYMDDILIHSKSESEHVKHVKDVLQKLKNANLIINQAKCEFHQSQVKFIGYHISEKGLTPCQENIDKVLQWKQPKNRKELRQFLGSVNYLRKFIPKTSQLTHPLNKLLKKDVRWKWTPTQTQAIENIKQCLVSPPVLRHFDFSKEILLETDASDVAVGAVLSQKHDDDKYYPVGYYSAKMSKAQLNYSVSDKEMLAIIKSLKHWRHYLESTIEPFKILTDHRNLIGRITNESEPENKRLARWQLFLQDFNFEINYRPGSANHIADALSRIVDETEPIPKDSEDNSINFVNQISI

SEQ ID NO: 198 - protein, 505 aa, synthetic construct
ISSSKHTLSQMNKVSNIVKEPELPDIYKEFKDITADTNTEKLPKPIKGLEFEVELTQENYRLPIRNYPLPPGKMQAMNDEINQGLKSGIIRESKAINACPVMFVPRKEGTLRMVVDYKPLNKYVKPNIYPLPLIEQLLAKIQGSTIFTKLDLKSAYHQIRVRKGDEHKLAFRCPRGVFEYLVMPYGISTAPAHFQYFINTILGEAKESHVVCYMDDILIHSKSESEHVKHVKDVLQKLKNANLIINQAKCEFHQSQVKFIGYHISEKGLTPCQENIDKVLQWKQPKNRKELRQFLGSVNYLRKFIPKTSQLTHPLNKLLKKDVRWKWTPTQTQAIENIKQCLVSPPVLRHFDFSKKILLETDVSDVAVGAVLSQKHDDDKYYPVGYYSAKMSKAQLNYSVSDKEMLAIIKSLKHWRHYLESTVEPFKILTDHRNLIGRITNESEPENKRLARWQLFLQDFNFEINYRPGSANHIADALSRIVDETEPIPKDSEDNSINFVNQISI

SEQ ID NO: 199 - protein, 505 aa, synthetic construct
ISSSKHTLSQMNKVSNIVKEPKLPDIYKEFKDITADTNTEKLPKPIKGLEFEVELTQENYRLPIRNYPLTPVKMQAMNDEINQGLKSGIIRESKAINACPVIFVPRKEGTLRMVVDYKPLNKYVKPNIYPLPLIEQLLTKIQGSTIFTKLDLKSAYHQIRVRKGDEHKLAFRCPRGVFEYLVMPYGISTAPAHFQYFINTILGEAKESHVVCYMDDILIHSKSESEHVKHVKDVLQKLKNANLIINQAKCEFHQSQVKFIGYHISEKGLTPCQENIDKVLQWKQPKNRKELRQFLGSVNYLRKFIPKTSQLTHPLNKLLKKDVRWKWTPTQTQAIENIKQCLVSPPVLRHFDFSKKILLETDVSDVAVGAVLSQKHDDDKYYPVGYYSAKMSKAQLNYSVSDKEMLAIIKSLEHWRHYLESTIEPFKILTDHRNLIGRITNESEPENKRLARWQLFLQDFNFEINYRPGSANHIADALSRIVDETEPIPKDNEDNSINFVNQISI

SEQ ID NO: 200 - protein, 526 aa, synthetic construct
ISSSKHTLSQMNKVSNIVKEPELPDIYKEFKDITADTNTEKLPKPIKGLEFEVELTQENYRLPIRNYPLTPVKMQAMNDEINQGLKSGIIRESKAINACPVIFVPRKEGTLRMVVDYKPLNKYVKPNIYPLPLIEQLLAKIQGSTIFTKLDLKSAYHQIRVRKGDEHKLAFRCPRGVFEYLVMPYGISTAPAHFQYFINTILGEAKESHVVCYMDDILIHSKSESEHVKHVKDVLQKLKNANLIINQAKCEFHQSQVKFIGYHISEKGLTPCQENIDKVLQWKQPKNRKELRQFLGSVNYLRKFIPKTSQLTHPLNKLLKKDVRWKWTPTQTQAIENIKQCLVSPPVLRHFDFSKKILLETDVSDVAVGAVLSQKHDDDKYYPVGYYSAKMSKAQLNYSVSDKEMLAIIKSLEHWRHYLESTIEPFKILTDHRNLIGRITNESEPENKRLARWQLFLQDFNFEINYRPGSANHIADALSRIVDETEPIPKDNEDNSINFVNQISISGGSKRTADGSEFEPKKKRKV

SEQ ID NO: 201 - protein, 505 aa, synthetic construct
ISSSKHTLSQMNKVSNIVKEPKLPDIYKEFKDITADTNTEKLPKPIKGLEFEVELTQENYRLPLRNYPLTPVKMQAMNDEINQGLKSGIIRESKAINACPVIFVPRKEGTLRMVVDYRPLNKYVKPNIYPLPLIEQLLTKIQGSTIFTKLDLKSAYHQIRVRKGDEHKLAFRCPRGVFEYLVMPYGIKTAPAHFQYFINTILGEAKESHVVCYMDDILIHSKSESEHVKHVKDVLQKLKNANLIINQAKCEFHQSQVKFLGYHISEKGLTPCQENIDKVLQWKQPKNRKELRQFLGQVNYLRKFIPKTSQLTHPLNKLLKKDVRWKWTPTQTQAIENIKQCLVSPPVLRHFDFSKKILLETDVSDVAVGAVLSQKHDDDKYYPVGYYSAKMSKAQLNYSVSDKEMLAIIKSLEHWRHYLESTIEPFKILTDHRNLIGRITNESEPENKRLARWQLFLQDFNFEINYRPGSANHIADALSRIVDETEPIPKDNEDNSINFVNQISI

SEQ ID NO: 202 - protein, 505 aa, synthetic construct
ISSSKHTLSQMNKVSNIVKEPELPDIYKEFKDITADTNTEKLPKPIKGLEFEVELTQENYRLPIRNYPLPPGKMQAMNDEINQGLKSGIIRESKAINACPVMFVPKKEGTLRMVVDYRPLNKYVKPNIYPLPLIEQLLAKIQGSTIFTKLDLKSAYHLIRVRKGDEHKLAFRCPRGVFEYLVMPYGISTAPAHFQYFINTILGEAKESHVVCYMDDILIHSKSESEHVKHVKDVLQKLKNANLIINQAKCEFHQSQVKFIGYHISEKGFTPCQENIDKVLQWKQPKNRKELRQFLGQVNYLRKFIPKTSQLTHPLNKLLKKDVRWKWTPTQTQAIENIKQCLVSPPVLRHFDFSKKILLETDASDVAVGAVLSQKHDDDKYYPVGYYSAKMSKAQLNYSVSDKEMLAIIKSLKHWRHYLESTIEPFKILTDHRNLIGRITNESEPENKRLARWQLFLQDFNFEINYRPGSANHIADALSRIVDETEPIPKDSEDNSINFVNQISI

SEQ ID NO: 203 - protein, 505 aa, synthetic construct
ISSSKHTLSQMNKVSNIVKEPELPDIYKEFKDITADTNTEKLPKPIKGLEFEVELTQENYRLPIRNYPLPPGKMQAMNDEINQGLKSGIIRESKAINACPVMFVPKKEGTLRMVVDYKPLNKYVKPNIYPLPLIEQLLAKIQGSTIFTKLDLKSAYHLIRVRKGDEHKLAFRCPRGVFEYLVMPYGIKTAPAHFQYFINTILGEAKESHVVCYMDDILIHSKSESEHVKHVKDVLQKLKNANLIINQAKCEFHQSQVKFIGYHISEKGFTPCQENIDKVLQWKQPKNRKELRQFLGSVNYLRKFIPKTSQLTHPLNKLLKKDVRWKWTPTQTQAIENIKQCLVSPPVLRHFDFSKKILLETDASDVAVGAVLSQKHDDDKYYPVGYYSAKMSKAQLNYSVSDKEMLAIIKSLKHWRHYLESTIEPFKILTDHRNLIGRITNESEPENKRLARWQLFLQDFNFEINYRPGSANHIADALSRIVDETEPIPKDSEDNSINFVNQISI

SEQ ID NO: 204 - protein, 505 aa, synthetic construct
ISSSKHTLSQMNKVSNIVKEPELPDIYKEFKDITADTNTEKLPKPIKGLEFEVELTQENYRLPIRNYPLPPGKMQAMNDEINQGLKSGIIRESKAINACPVMFVPKKEGTLRMVVDYKPLNKYVKPNIYPLPLIEQLLAKIQGSTIFTKLDLKSAYHLIRVRKGDEHKLAFRCPRGVFEYLVMPYGISTAPAHFQYFINTILGEAKESHVVCYMDDILIHSKSESEHVKHVKDVLQKLKNANLIINQAKCEFHQSQVKFLGYHISEKGFTPCQENIDKVLQWKQPKNRKELRQFLGSVNYLRKFIPKTSQLTHPLNKLLKKDVRWKWTPTQTQAIENIKQCLVSPPVLRHFDFSKKILLETDASDVAVGAVLSQKHDDDKYYPVGYYSAKMSKAQLNYSVSDKEMLAIIKSLKHWRHYLESTIEPFKILTDHRNLIGRITNESEPENKRLARWQLFLQDFNFEINYRPGSANHIADALSRIVDETEPIPKDSEDNSINFVNQISI

SEQ ID NO: 205 - protein, 505 aa, synthetic construct
ISSSKHTLSQMNKVSNIVKEPELPDIYKEFKDITADTNTEKLPKPIKGLEFEVELTQENYRLPIRNYPLPPGKMQAMNDEINQGLKSGIIRESKAINACPVMFVPKKEGTLRMVVDYKPLNKYVKPNIYPLPLIEQLLAKIQGSTIFTKLDLKSAYHLIRVRKGDEHKLAFRCPRGVFEYLVMPYGISTAPAHFQYFINTILGEAKESHVVCYMDDILIHSKSESEHVKHVKDVLQKLKNANLIINQAKCEFHQSQVKFIGYHISEKGFTPCQENIDKVLQWKQPKNQKELRQFLGSVNYLRKFIPKTSQLTHPLNKLLKKDVRWKWTPTQTQAIENIKQCLVSPPVLRHFDFSKKILLETDASDVAVGAVLSQKHDDDKYYPVGYYSAKMSKAQLNYSVSDKEMLAIIKSLKHWRHYLESTIEPFKILTDHRNLIGRITNESEPENKRLARWQLFLQDFNFEINYRPGSANHIADALSRIVDETEPIPKDSEDNSINFVNQISI

SEQ ID NO: 206 - protein, 505 aa, synthetic construct
ISSSKHTLSQMNKVSNIVKEPELPDIYKEFKDITADTNTEKLPKPIKGLEFEVELTQENYRLPIRNYPLPPGKMQAMNDEINQGLKSGIIRESKAINACPVMFVPKKEGTLRMVVDYKPLNKYVKPNIYPLPLIEQLLAKIQGSTIFTKLDLKSAYHLIRVRKGDEHKLAFRCPRGVFEYLVMPYGISTAPAHFQYFINTILGEAKESHVVCYMDDILIHSKSESEHVKHVKDVLQKLKNANLIINQAKCEFHQSQVKFIGYHISEKGFTPCQENIDKVLQWKQPKNRKELRKFLGSVNYLRKFIPKTSQLTHPLNKLLKKDVRWKWTPTQTQAIENIKQCLVSPPVLRHFDFSKKILLETDASDVAVGAVLSQKHDDDKYYPVGYYSAKMSKAQLNYSVSDKEMLAIIKSLKHWRHYLESTIEPFKILTDHRNLIGRITNESEPENKRLARWQLFLQDFNFEINYRPGSANHIADALSRIVDETEPIPKDSEDNSINFVNQISI

SEQ ID NO: 207 - protein, 505 aa, synthetic construct
ISSSKHTLSQMNKVSNIVKEPELPDIYKEFKDITADTNTEKLPKPIKGLEFEVELTQENYRLPLRNYPLPPGKMQAMNDEINQGLKSGIIRESKAINACPVMFVPKKEGTLRMVVDYKPLNKYVKPNIYPLPLIEQLLAKIQGSTIFTKLDLKSAYHLIRVRKGDEHKLAFRCPRGVFEYLVMPYGISTAPAHFQYFINTILGEAKESHVVCYMDDILIHSKSESEHVKHVKDVLQKLKNANLIINQAKCEFHQSQVKFIGYHISEKGFTPCQENIDKVLQWKQPKNRKELRQFLGSVNYLRKFIPKTSQLTHPLNKLLKKDVRWKWTPTQTQAIENIKQCLVSPPVLRHFDFSKKILLETDASDVAVGAVLSQKHDDDKYYPVGYYSAKMSKAQLNYSVSDKEMLAIIKSLKHWRHYLESTIEPFKILTDHRNLIGRITNESEPENKRLARWQLFLQDFNFEINYRPGSANHIADALSRIVDETEPIPKDSEDNSINFVNQISI

SEQ ID NO: 208 - protein, 505 aa, synthetic construct
ISSSKHTLSQMNKVSNIVKEPELPDIYKEFKDITADTNTEKLPKPIKGLEFEVELTQENYRLPWRNYPLPPGKMQAMNDEINQGLKSGIIRESKAINACPVMFVPKKEGTLRMVVDYKPLNKYVKPNIYPLPLIEQLLAKIQGSTIFTKLDLKSAYHLIRVRKGDEHKLAFRCPRGVFEYLVMPYGISTAPAHFQYFINTILGEAKESHVVCYMDDILIHSKSESEHVKHVKDVLQKLKNANLIINQAKCEFHQSQVKFIGYHISEKGFTPCQENIDKVLQWKQPKNRKELRQFLGSVNYLRKFIPKTSQLTHPLNKLLKKDVRWKWTPTQTQAIENIKQCLVSPPVLRHFDFSKKILLETDASDVAVGAVLSQKHDDDKYYPVGYYSAKMSKAQLNYSVSDKEMLAIIKSLKHWRHYLESTIEPFKILTDHRNLIGRITNESEPENKRLARWQLFLQDFNFEINYRPGSANHIADALSRIVDETEPIPKDSEDNSINFVNQISI

SEQ ID NO: 209 - protein, 505 aa, synthetic construct
ISSSKHTLSQMNKVSNIVKEPELPDIYKEFKDITADTNTEKLPKPIKGLEFEVELTQENYRLPIRNYPLPPGKMQAMNDEINQGLKSGIIRESKAINACPVMFVPKKEGTLRMVVDYKPLNKYVKPNIYPLPLIEQLLAKIQGSTIFTKLDLKSAYHLIRVRKGDEHKLAFRCPRGVFEYLVMPYGISTAPAHFQYFINTILGEAKESHVVCYMDDILIHSKSESEHVKHVKDVLQKLKNANLIINQAKCEFHQSQVKFIGYHISEKGFTPCQENIDKVLQWKQPKNRKELRQFLGSVNYLRKFIPKTSQLTHPLQKLLKKDVRWKWTPTQTQAIENIKQCLVSPPVLRHFDFSKKILLETDASDVAVGAVLSQKHDDDKYYPVGYYSAKMSKAQLNYSVSDKEMLAIIKSLKHWRHYLESTIEPFKILTDHRNLIGRITNESEPENKRLARWQLFLQDFNFEINYRPGSANHIADALSRIVDETEPIPKDSEDNSINFVNQISI

SEQ ID NO: 210 - protein, 505 aa, synthetic construct
ISSSKHTLSQMNKVSNIVKEPELPDIYKEFKDITADTNTEKLPKPIKGLEFEVELTQENYRLPIRNYPLPPGKMQAMNDEINQGLKSGIIRESKAINACPVMFVPKKEGTLRMVVDYKPLNKYVKPNIYPLPLIEQLLAKIQGSTIFTKLDLKSAYHLIRVRKGDEHKLAFRCPRGVFEYLVMPYGISTAPAHFQYFINTILGEAKESHVVCYMDDILIHSKSESEHVKHVKDVLQKLKNANLIINQAKCEFHQSQVKFIGYHISEKGFTPCQENIDKVLQWKQPKNRKELRQFLGSVNYLRKFIPKTSQLTHPLNKLLKRDVRWKWTPTQTQAIENIKQCLVSPPVLRHFDFSKKILLETDASDVAVGAVLSQKHDDDKYYPVGYYSAKMSKAQLNYSVSDKEMLAIIKSLKHWRHYLESTIEPFKILTDHRNLIGRITNESEPENKRLARWQLFLQDFNFEINYRPGSANHIADALSRIVDETEPIPKDSEDNSINFVNQISI

SEQ ID NO: 211 - protein, 505 aa, synthetic construct
ISSSKHTLSQMNKVSNIVKEPELPDIYKEFKDITADTNTEKLPKPIKGLEFEVELTQENYRLPIRNYPLPPGKMQAMNDEINQGLKSGIIRESKAINACPVMFVPKKEGTLRMVVDYKPLNKYVKPNIYPLPNIEQLLAKIQGSTIFTKLDLKSAYHLIRVRKGDEHKLAFRCPRGVFEYLVMPYGISTAPAHFQYFINTILGEAKESHVVCYMDDILIHSKSESEHVKHVKDVLQKLKNANLIINQAKCEFHQSQVKFIGYHISEKGFTPCQENIDKVLQWKQPKNRKELRQFLGSVNYLRKFIPKTSQLTHPLNKLLKKDVRWKWTPTQTQAIENIKQCLVSPPVLRHFDFSKKILLETDASDVAVGAVLSQKHDDDKYYPVGYYSAKMSKAQLNYSVSDKEMLAIIKSLKHWRHYLESTIEPFKILTDHRNLIGRITNESEPENKRLARWQLFLQDFNFEINYRPGSANHIADALSRIVDETEPIPKDSEDNSINFVNQISI

SEQ ID NO: 212 - protein, 505 aa, synthetic construct
ISSSKHTLSQMNKVSNIVKEPELPDIYKEFKDITADTNTEKLPKPIKGLEFEVELTQENYRLPIRNYPLPPGKMQAMNDEINQGLKSGIIRESKAINACPVMFVPKKEGTLRMVVDYRPLNKYVKPNIYPLPLIEQLLAKIQGSTIFTKLDLKSAYHLIRVRKGDEHKLAFRCPRGVFEYLVMPYGISTAPAHFQYFINTILGEAKESHVVCYMDDILIHSKSESEHVKHVKDVLQKLKNANLIINQAKCEFHQSQVKFIGYHISEKGFTPCQENIDKVLQWKQPKNRKELRQFLGSVNYLRKFIPKTSQLTHPLNKLLKKDVRWKWTPTQTQAIENIKQCLVSPPVLRHFDFSKKILLETDASDVAVGAVLSQKHDDDKYYPVGYYSAKMSKAQLNYSVSDKEMLAIIKSLKHWRHYLESTIEPFKILTDHRNLIGRITNESEPENKRLARWQLFLQDFNFEINYRPGSANHIADALSRIVDETEPIPKDSEDNSINFVNQISI

SEQ ID NO: 213 - protein, 505 aa, synthetic construct
ISSSKHTLSQMNKVSNIVKEPELPDIYKEFKDITADTNTEKLPKPIKGLEFEVELTQENYRLPIRNYPLPPGKMQAMNDEINQGLKSGIIRESKAINACPVMFVPKKEGTLRMVVDYKPLNKYVKPNIYPLPLIEQLLAKIQGSTIFTKLDLKSAYHLIRVRKGDEHKLAFRCPRGVFEYLVMPYGISTAPAHFQYFINTILGEAKESHVVCYMDDILIHSKSESEHVKHVKDVLQKLKNANLIINQAKCEFHQSQVKFIGYHISEKGFTPCQENIDKVLQWKQPKNRKELRQFLGQVNYLRKFIPKTSQLTHPLNKLLKKDVRWKWTPTQTQAIENIKQCLVSPPVLRHFDFSKKILLETDASDVAVGAVLSQKHDDDKYYPVGYYSAKMSKAQLNYSVSDKEMLAIIKSLKHWRHYLESTIEPFKILTDHRNLIGRITNESEPENKRLARWQLFLQDFNFEINYRPGSANHIADALSRIVDETEPIPKDSEDNSINFVNQISI

SEQ ID NO: 214 - protein, 693 aa, synthetic construct
APLEEEYRLFLEAPIQNVTLLEQWKREIPKVWAEINPPGLASTQAPIHVQLLSTALPVRVRQYPITLEAKRSLRETIRKFRAAGILRPVHSPWNTPLLPVRKSGTSEYRMVQDLREVNKRVETIHPTVPNPYTLLSLLPPDRIWYSVLDLKDAFFCIPLAPESQLIFAFEWADAEEGESGQLTWTRLPQGFKNSPTLFNEALNRDLQGFRLDHPSVSLLQYVDDLLIAADTQAACLSATRDLLMTLAELGYRVSGKKAQLCQEEVTYLGFKIHKGSRSLSNSRTQAILQIPVPKTKRQVREFLGTIGYCRLWIPGFAELAQPLYAATRGGNDPLVWGEKEEEAFQSLKLALTQPPALALPSLDKPFQLFVEETSGAAKGVLTQALGPWKRPVAYLSKRLDPVAAGWPRCLRAIAAAALLTREASKLTFGQDIEITSSHNLESLLRSPPDKWLTNARITQYQVLLLDPPRVRFKQTAALNPATLLPETDDTLPIHHCLDTLDSLTSTRPDLTDQPLAQAEATLFTDGSSYIRDGKRYAGAAVVTLDSVIWAEPLPIGTSAQKAELIALTKALEWSKDKSVNIYTDSRYAFATLHVHGMIYRERGLLTAGGKAIKNAPEILALLTAVWLPKRVAVMHCKGHQKDDAPTSTGNRRADEVAREVAIRPLSTQATISDAPDMPDTETPQYSNVEEALG

SEQ ID NO: 215 - protein, 693 aa, synthetic construct
APLEEEYRLFLEAPIQNVTLLEQWKREIPKVWAEINPPGLASTQAPIHVQLLSTALPVRVRQYPITLEAKRSLRETIRKFRAAGILRPVHSPWNTPLLPVRKSGTSEYRMVQDLREVNKRVETIHPTVPNPYTLLSLLPPDRIWYSVLDLKDAFFCIPLAPESQLIFAFEWADAEEGESGQLTWTRLPQGFKNSPTLFDEALNRDLQGFRLDHPSVSLLQYVDDLLIAADTQAACLSATRDLLMTLAELGYRVSGKKAQLCQEEVTYLGFKIHKGSRSLSNSRTQAILQIPVPKTKRQVREFLGKIGYCRLWIPGFAELAQPLYAATRGGNDPLVWGEKEEEAFQSLKLALTQPPALALPSLDKPFQLFVEETSGAAKGVLTQALGPWKRPVAYLSKRLDPVAAGWPRCLRAIAAAALLTREASKLTFGQDIEITSSHNLESLLRSPPDKWLTNARITQYQVLLLDPPRVRFKQTAALNPATLLPETDDTLPIHHCLDTLDSLTSTRPDLTDQPLAQAEATLFTDGSSYIRDGKRYAGAAVVTLDSVIWAEPLPIGTSAQKAELIALTKALEWSKDKSVNIYTDSRYAFATLHVHGMIYRERGLLTAGGKAIKNAPEILALLTAVWLPKRVAVMHCKGHQKDDAPTSTGNRRADEVAREVAIRPLSTQATISDAPDMPDTETPQYSNVEEALG

SEQ ID NO: 216 - protein, 693 aa, synthetic construct
APLEEEYRLFLEAPIQNVTLLEQWKREIPKVWAEINPPGLASTQAPIHVQLLSTALPVRVRQYPITLEAKRSLRETIRKFRAAGILRPVHSPWNTPLLPVRKSGTSEYRMVQDLREVNKRVETIHPTVPNPYTLLSLLPPDRIWYSVLDLKDAFFCIPLAPESQLIFAFEWADAEEGESGQLTWTRLPQGFKNSPTLFDEALNRDLQGFRLDHPSVSLLQYVDDLLIAADTQAACLSATRDLLMTLAELGYRVSGKKAQLCQEEVTYLGFKIHKGSRSLSNSRTQAILQIPVPKTKRQVREFLGTIGYCRLFIPGFAELAQPLYAATRGGNDPLVWGEKEEEAFQSLKLALTQPPALALPSLDKPFQLFVEETSGAAKGVLTQALGPWKRPVAYLSKRLDPVAAGWPRCLRAIAAAALLTREASKLTFGQDIEITSSHNLESLLRSPPDKWLTNARITQYQVLLLDPPRVRFKQTAALNPATLLPETDDTLPIHHCLDTLDSLTSTRPDLTDQPLAQAEATLFTDGSSYIRDGKRYAGAAVVTLDSVIWAEPLPIGTSAQKAELIALTKALEWSKDKSVNIYTDSRYAFATLHVHGMIYRERGLLTAGGKAIKNAPEILALLTAVWLPKRVAVMHCKGHQKDDAPTSTGNRRADEVAREVAIRPLSTQATISDAPDMPDTETPQYSNVEEALG

SEQ ID NO: 217 - protein, 693 aa, synthetic construct
APLEEEYRLFLEAPIQNVTLLEQWKREIPKVWAEINPPGLASTQAPIHVQLLSTALPVRVRQYPITLEAKRSLRETIRKFRAAGILRPVHSPWNTPLLPVRKSGTSEYRMVQDLREVNKRVETIHPTVPNPYTLLSLLPPDRIWYSVLDLKDAFFCIPLAPESQLIFAFEWADAEEGESGQLTWTRLPQGFKNSPTLFDEALNRDLQGFRLDHPSVSLLQYVDDLLIAADTQAACLSATRDLLMTLAELGYRVSGKKAQLCQEEVTYLGFKIHKGSRSLSNSRTQAILQIPVPKTKRQVREFLGTIGYCRLWIPGFAELAQPLYAATRPGNDPLVWGEKEEEAFQSLKLALTQPPALALPSLDKPFQLFVEETSGAAKGVLTQALGPWKRPVAYLSKRLDPVAAGWPRCLRAIAAAALLTREASKLTFGQDIEITSSHNLESLLRSPPDKWLTNARITQYQVLLLDPPRVRFKQTAALNPATLLPETDDTLPIHHCLDTLDSLTSTRPDLTDQPLAQAEATLFTDGSSYIRDGKRYAGAAVVTLDSVIWAEPLPIGTSAQKAELIALTKALEWSKDKSVNIYTDSRYAFATLHVHGMIYRERGLLTAGGKAIKNAPEILALLTAVWLPKRVAVMHCKGHQKDDAPTSTGNRRADEVAREVAIRPLSTQATISDAPDMPDTETPQYSNVEEALG

SEQ ID NO: 218 - protein, 693 aa, synthetic construct
APLEEEYRLFLEAPIQNVTLLEQWKREIPKVWAEINPPGLASTQAPIHVQLLSTALPVRVRQYPITLEAKRSLRETIRKFRAAGILRPVHSPWNTPLLPVRKSGTSEYRMVQDLREVNKRVETIHPTVPNPYTLLSLLPPDRIWYSVLDLKDAFFCIPLAPESQLIFAFEWADAEEGESGQLTWTRLPQGFKNSPTLFDEALNRDLQGFRLDHPSVSLLQYVDDLLIAADTQAACLSATRDLLMTLAELGYRVSGKKAQLCQEEVTYLGFKIHKGSRSLSNSRTQAILQIPVPKTKRQVREFLGTIGYCRLWIPGFAELAQPLYAATRGGNDPLVWGEKEEEAFQSLKLALTQPPALALPSLDKPFQLFVEETSGAAKGVLTQALGPWKRPVAYLSKRLDPVAAGWPRCLRAIAAAALLTREASKLTFGQDIEITSSHNLESLLRSPPDKWLTNARITQYQVLLLDPPRVRFKQTAALNPATLLPETDDTLPIHHCLDTLDSLTSTRPDLTDQPLAQAEATLFTDGSSYIRDGKRYAGAAVVTLDSVIWAEPLPIGTSAQKAELIALTKALEWSKDKSVNIYTDSRYAFATLHVHGMIYRERGWLTAGGKAIKNAPEILALLTAVWLPKRVAVMHCKGHQKDDAPTSTGNRRADEVAREVAIRPLSTQATISDAPDMPDTETPQYSNVEEALG

SEQ ID NO: 219 - protein, 693 aa, synthetic construct
APLEEEYRLFLEAPIQNVTLLEQWKREIPKVWAEINPPGLASTQAPIHVQLLSTALPVRVRQYPITLEAKRSLRETIRKFRAAGILRPVHSPWNTPLLPVRKSGTSEYRMVQDLREVNKRVETIHPTVPNPYTLLSLLPPDRIWYSVLDLKDAFFCIPLAPESQLIFAFEWADAEEGESGQLTWTRLPQGFKNSPTLFNEALNRDLQGFRLDHPSVSLLQYVDDLLIAADTQAACLSATRDLLMTLAELGYRVSGKKAQLCQEEVTYLGFKIHKGSRSLSNSRTQAILQIPVPKTKRQVREFLGKIGYCRLFIPGFAELAQPLYAATRPGNDPLVWGEKEEEAFQSLKLALTQPPALALPSLDKPFQLFVEETSGAAKGVLTQALGPWKRPVAYLSKRLDPVAAGWPRCLRAIAAAALLTREASKLTFGQDIEITSSHNLESLLRSPPDKWLTNARITQYQVLLLDPPRVRFKQTAALNPATLLPETDDTLPIHHCLDTLDSLTSTRPDLTDQPLAQAEATLFTDGSSYIRDGKRYAGAAVVTLDSVIWAEPLPIGTSAQKAELIALTKALEWSKDKSVNIYTDSRYAFATLHVHGMIYRERGWLTAGGKAIKNAPEILALLTAVWLPKRVAVMHCKGHQKDDAPTSTGNRRADEVAREVAIRPLSTQATISDAPDMPDTETPQYSNVEEALG

SEQ ID NO: 220 - protein, 678 aa, synthetic construct
MNLEEEYRLHEKPVPPSIDPSWLQLFPMVWAEKAGMGLANQVPPVVVELKSDASPVAVRQYPMSKEAREGIRPHIQRFLDLGILVPCQSPWNTPLLPVKKPGTNDYRPVQDLREVNKRVQDIHPTVPNPYNLLSSLPPSHTWYSVLDLKDAFFCLKLHPNSQPLFAFEWRDPEKGNTGQLTWTRLPQGFKNSPTLFNEALHRDLASFRALNPQVVMLQYVDDLLVAAPTYRDCKEGTRRLLQELSKLGYRVSAKKAQLCREEVTYLGYLLKGGKRWLTPARKATVMKIPTPTTPRQVREFLGTAGFCRLWIPGFASLAAPLYPLTREKVPFTWTEAHQEAFGRIKEALLSAPALALPDLTKPFALYVDEKEGVARGVLTQTLGPWRRPVAYLSKKLDPVASGWPTCLKAIAAVALLLKDADKLTLGQNVLVIAPHNLESIVRQPPDRWMTNARMTHYQSLLLNERVSFAPPAILNPATLLPVESDDTPIHICSEILAEETGTRPDLRDQPLPGVPAWYTDGSSFIMDGRRQAGAAIVDNKRTVWASNLPEGTSAQKAELIALTQALRLAEGKSINIYTDSRYAFATAHVHGAIYKQRGLLTSAGKDIKNKEEILALLEAIHLPKRVAIIHCPGHQRGTDPVATGNRKADEAAKQAAQSTRILTETTKNQEHFEPTRGK

SEQ ID NO: 221 - protein, 678 aa, synthetic construct
MNLEEEYRLHEKPVPPSIDPSWLQLFPMVWAEKAGMGLANQVPPVVVELKSDASPVAVRQYPMSKEAREGIRPHIQRFLDLGILVPCQSPWNTPLLPVKKPGTNDYRPVQDLREVNKRVQDIHPTVPNPYNLLSSLPPSHTWYSVLDLKDAFFCLKLHPNSQPLFAFEWRDPEKGNTGQLTWTRLPQGFKNSPTLFDEALHRDLASFRALNPQVVMLQYVDDLLVAAPTYRDCKEGTRRLLQELSKLGYRVSAKKAQLCREEVTYLGYLLKGGKRWLTPARKATVMKIPTPTTPRQVREFLGKAGFCRLWIPGFASLAAPLYPLTREKVPFTWTEAHQEAFGRIKEALLSAPALALPDLTKPFALYVDEKEGVARGVLTQTLGPWRRPVAYLSKKLDPVASGWPTCLKAIAAVALLLKDADKLTLGQNVLVIAPHNLESIVRQPPDRWMTNARMTHYQSLLLNERVSFAPPAILNPATLLPVESDDTPIHICSEILAEETGTRPDLRDQPLPGVPAWYTDGSSFIMDGRRQAGAAIVDNKRTVWASNLPEGTSAQKAELIALTQALRLAEGKSINIYTDSRYAFATAHVHGAIYKQRGLLTSAGKDIKNKEEILALLEAIHLPKRVAIIHCPGHQRGTDPVATGNRKADEAAKQAAQSTRILTETTKNQEHFEPTRGK

SEQ ID NO: 222 - protein, 678 aa, synthetic construct
MNLEEEYRLHEKPVPPSIDPSWLQLFPMVWAEKAGMGLANQVPPVVVELKSDASPVAVRQYPMSKEAREGIRPHIQRFLDLGILVPCQSPWNTPLLPVKKPGTNDYRPVQDLREVNKRVQDIHPTVPNPYNLLSSLPPSHTWYSVLDLKDAFFCLKLHPNSQPLFAFEWRDPEKGNTGQLTWTRLPQGFKNSPTLFDEALHRDLASFRALNPQVVMLQYVDDLLVAAPTYRDCKEGTRRLLQELSKLGYRVSAKKAQLCREEVTYLGYLLKGGKRWLTPARKATVMKIPTPTTPRQVREFLGTAGFCRLFIPGFASLAAPLYPLTREKVPFTWTEAHQEAFGRIKEALLSAPALALPDLTKPFALYVDEKEGVARGVLTQTLGPWRRPVAYLSKKLDPVASGWPTCLKAIAAVALLLKDADKLTLGQNVLVIAPHNLESIVRQPPDRWMTNARMTHYQSLLLNERVSFAPPAILNPATLLPVESDDTPIHICSEILAEETGTRPDLRDQPLPGVPAWYTDGSSFIMDGRRQAGAAIVDNKRTVWASNLPEGTSAQKAELIALTQALRLAEGKSINIYTDSRYAFATAHVHGAIYKQRGLLTSAGKDIKNKEEILALLEAIHLPKRVAIIHCPGHQRGTDPVATGNRKADEAAKQAAQSTRILTETTKNQEHFEPTRGK

SEQ ID NO: 223 - protein, 678 aa, synthetic construct
MNLEEEYRLHEKPVPPSIDPSWLQLFPMVWAEKAGMGLANQVPPVVVELKSDASPVAVRQYPMSKEAREGIRPHIQRFLDLGILVPCQSPWNTPLLPVKKPGTNDYRPVQDLREVNKRVQDIHPTVPNPYNLLSSLPPSHTWYSVLDLKDAFFCLKLHPNSQPLFAFEWRDPEKGNTGQLTWTRLPQGFKNSPTLFDEALHRDLASFRALNPQVVMLQYVDDLLVAAPTYRDCKEGTRRLLQELSKLGYRVSAKKAQLCREEVTYLGYLLKGGKRWLTPARKATVMKIPTPTTPRQVREFLGTAGFCRLWIPGFASLAAPLYPLTRPKVPFTWTEAHQEAFGRIKEALLSAPALALPDLTKPFALYVDEKEGVARGVLTQTLGPWRRPVAYLSKKLDPVASGWPTCLKAIAAVALLLKDADKLTLGQNVLVIAPHNLESIVRQPPDRWMTNARMTHYQSLLLNERVSFAPPAILNPATLLPVESDDTPIHICSEILAEETGTRPDLRDQPLPGVPAWYTDGSSFIMDGRRQAGAAIVDNKRTVWASNLPEGTSAQKAELIALTQALRLAEGKSINIYTDSRYAFATAHVHGAIYKQRGLLTSAGKDIKNKEEILALLEAIHLPKRVAIIHCPGHQRGTDPVATGNRKADEAAKQAAQSTRILTETTKNQEHFEPTRGK

SEQ ID NO: 224 - protein, 678 aa, synthetic construct
MNLEEEYRLHEKPVPPSIDPSWLQLFPMVWAEKAGMGLANQVPPVVVELKSDASPVAVRQYPMSKEAREGIRPHIQRFLDLGILVPCQSPWNTPLLPVKKPGTNDYRPVQDLREVNKRVQDIHPTVPNPYNLLSSLPPSHTWYSVLDLKDAFFCLKLHPNSQPLFAFEWRDPEKGNTGQLTWTRLPQGFKNSPTLFDEALHRDLASFRALNPQVVMLQYVDDLLVAAPTYRDCKEGTRRLLQELSKLGYRVSAKKAQLCREEVTYLGYLLKGGKRWLTPARKATVMKIPTPTTPRQVREFLGTAGFCRLWIPGFASLAAPLYPLTREKVPFTWTEAHQEAFGRIKEALLSAPALALPDLTKPFALYVDEKEGVARGVLTQTLGPWRRPVAYLSKKLDPVASGWPTCLKAIAAVALLLKDADKLTLGQNVLVIAPHNLESIVRQPPDRWMTNARMTHYQSLLLNERVSFAPPAILNPATLLPVESDDTPIHICSEILAEETGTRPDLRDQPLPGVPAWYTDGSSFIMDGRRQAGAAIVDNKRTVWASNLPEGTSAQKAELIALTQALRLAEGKSINIYTDSRYAFATAHVHGAIYKQRGWLTSAGKDIKNKEEILALLEAIHLPKRVAIIHCPGHQRGTDPVATGNRKADEAAKQAAQSTRILTETTKNQEHFEPTRGK

SEQ ID NO: 225 - protein, 678 aa, synthetic construct
MNLEEEYRLHEKPVPPSIDPSWLQLFPMVWAEKAGMGLANQVPPVVVELKSDASPVAVRQYPMSKEAREGIRPHIQRFLDLGILVPCQSPWNTPLLPVKKPGTNDYRPVQDLREVNKRVQDIHPTVPNPYNLLSSLPPSHTWYSVLDLKDAFFCLKLHPNSQPLFAFEWRDPEKGNTGQLTWTRLPQGFKNSPTLFNEALHRDLASFRALNPQVVMLQYVDDLLVAAPTYRDCKEGTRRLLQELSKLGYRVSAKKAQLCREEVTYLGYLLKGGKRWLTPARKATVMKIPTPTTPRQVREFLGKAGFCRLFIPGFASLAAPLYPLTRPKVPFTWTEAHQEAFGRIKEALLSAPALALPDLTKPFALYVDEKEGVARGVLTQTLGPWRRPVAYLSKKLDPVASGWPTCLKAIAAVALLLKDADKLTLGQNVLVIAPHNLESIVRQPPDRWMTNARMTHYQSLLLNERVSFAPPAILNPATLLPVESDDTPIHICSEILAEETGTRPDLRDQPLPGVPAWYTDGSSFIMDGRRQAGAAIVDNKRTVWASNLPEGTSAQKAELIALTQALRLAEGKSINIYTDSRYAFATAHVHGAIYKQRGWLTSAGKDIKNKEEILALLEAIHLPKRVAIIHCPGHQRGTDPVATGNRKADEAAKQAAQSTRILTETTKNQEHFEPTRGK

SEQ ID NO: 226 - protein, 672 aa, synthetic construct
LNLEEEYRLHEKPVPSSIDPSWLQLFPTVWAERAGMGLANQVPPVVVELRSGASPVAVRQYPMSKEAREGIRPHIQRFLDLGVLVPCQSPWNTPLLPVKKPGTNDYRPVQDLREINKRVQDIHPTVPNPYNLLSSLPPSHTWYSVLDLKDAFFCLKLHPNSQPLFAFEWRDPEKGNTGQLTWTRLPQGFKNSPTLFNEALHRDLAPFRALNPQVVLLQYVDDLLVAAPTYRDCKEGTQKLLQELSKLGYRVSAKKAQLCQKEVTYLGYLLKEGKRWLTPARKATVMKIPPPTTPRQVREFLGTAGFCRLWIPGFASLAAPLYPLTKESIPFIWTEEHQKAFDRIKEALLSAPALALPDLTKPFTLYVDERAGVARGVLTQTLGPWRRPVAYLSKKLDPVASGWPTCLKAVAAVALLLKDADKLTLGQNVTVIASHSLESIVRQPPDRWMTNARMTHYQSLLLNERVSFAPPAVLNPATLLPVESEATPVHRCSEILAEETGTRRDLKDQPLPGVPAWYTDGSSFIAEGKRRAGAAIVDGKRTVWASSLPEGTSAQKAELVALTQALRLAEGKDINIYTDSRYAFATAHIHGAIYKQRGLLTSAGKDIKNKEEILALLEAIHLPKRVAIIHCPGHQKGNDPVATGNRRADEAAKQAALSTRVLAETTKPQELI

SEQ ID NO: 227 - protein, 672 aa, synthetic construct
LNLEEEYRLHEKPVPSSIDPSWLQLFPTVWAERAGMGLANQVPPVVVELRSGASPVAVRQYPMSKEAREGIRPHIQRFLDLGVLVPCQSPWNTPLLPVKKPGTNDYRPVQDLREINKRVQDIHPTVPNPYNLLSSLPPSHTWYSVLDLKDAFFCLKLHPNSQPLFAFEWRDPEKGNTGQLTWTRLPQGFKNSPTLFDEALHRDLAPFRALNPQVVLLQYVDDLLVAAPTYRDCKEGTQKLLQELSKLGYRVSAKKAQLCQKEVTYLGYLLKEGKRWLTPARKATVMKIPPPTTPRQVREFLGKAGFCRLWIPGFASLAAPLYPLTKESIPFIWTEEHQKAFDRIKEALLSAPALALPDLTKPFTLYVDERAGVARGVLTQTLGPWRRPVAYLSKKLDPVASGWPTCLKAVAAVALLLKDADKLTLGQNVTVIASHSLESIVRQPPDRWMTNARMTHYQSLLLNERVSFAPPAVLNPATLLPVESEATPVHRCSEILAEETGTRRDLKDQPLPGVPAWYTDGSSFIAEGKRRAGAAIVDGKRTVWASSLPEGTSAQKAELVALTQALRLAEGKDINIYTDSRYAFATAHIHGAIYKQRGLLTSAGKDIKNKEEILALLEAIHLPKRVAIIHCPGHQKGNDPVATGNRRADEAAKQAALSTRVLAETTKPQELI

SEQ ID NO: 228 - protein, 672 aa, synthetic construct
LNLEEEYRLHEKPVPSSIDPSWLQLFPTVWAERAGMGLANQVPPVVVELRSGASPVAVRQYPMSKEAREGIRPHIQRFLDLGVLVPCQSPWNTPLLPVKKPGTNDYRPVQDLREINKRVQDIHPTVPNPYNLLSSLPPSHTWYSVLDLKDAFFCLKLHPNSQPLFAFEWRDPEKGNTGQLTWTRLPQGFKNSPTLFDEALHRDLAPFRALNPQVVLLQYVDDLLVAAPTYRDCKEGTQKLLQELSKLGYRVSAKKAQLCQKEVTYLGYLLKEGKRWLTPARKATVMKIPPPTTPRQVREFLGTAGFCRLFIPGFASLAAPLYPLTKESIPFIWTEEHQKAFDRIKEALLSAPALALPDLTKPFTLYVDERAGVARGVLTQTLGPWRRPVAYLSKKLDPVASGWPTCLKAVAAVALLLKDADKLTLGQNVTVIASHSLESIVRQPPDRWMTNARMTHYQSLLLNERVSFAPPAVLNPATLLPVESEATPVHRCSEILAEETGTRRDLKDQPLPGVPAWYTDGSSFIAEGKRRAGAAIVDGKRTVWASSLPEGTSAQKAELVALTQALRLAEGKDINIYTDSRYAFATAHIHGAIYKQRGLLTSAGKDIKNKEEILALLEAIHLPKRVAIIHCPGHQKGNDPVATGNRRADEAAKQAALSTRVLAETTKPQELI

SEQ ID NO: 229 - protein, 672 aa, synthetic construct
LNLEEEYRLHEKPVPSSIDPSWLQLFPTVWAERAGMGLANQVPPVVVELRSGASPVAVRQYPMSKEAREGIRPHIQRFLDLGVLVPCQSPWNTPLLPVKKPGTNDYRPVQDLREINKRVQDIHPTVPNPYNLLSSLPPSHTWYSVLDLKDAFFCLKLHPNSQPLFAFEWRDPEKGNTGQLTWTRLPQGFKNSPTLFDEALHRDLAPFRALNPQVVLLQYVDDLLVAAPTYRDCKEGTQKLLQELSKLGYRVSAKKAQLCQKEVTYLGYLLKEGKRWLTPARKATVMKIPPPTTPRQVREFLGTAGFCRLWIPGFASLAAPLYPLTKPSIPFIWTEEHQKAFDRIKEALLSAPALALPDLTKPFTLYVDERAGVARGVLTQTLGPWRRPVAYLSKKLDPVASGWPTCLKAVAAVALLLKDADKLTLGQNVTVIASHSLESIVRQPPDRWMTNARMTHYQSLLLNERVSFAPPAVLNPATLLPVESEATPVHRCSEILAEETGTRRDLKDQPLPGVPAWYTDGSSFIAEGKRRAGAAIVDGKRTVWASSLPEGTSAQKAELVALTQALRLAEGKDINIYTDSRYAFATAHIHGAIYKQRGLLTSAGKDIKNKEEILALLEAIHLPKRVAIIHCPGHQKGNDPVATGNRRADEAAKQAALSTRVLAETTKPQELI

SEQ ID NO: 230 - protein, 672 aa, synthetic construct
LNLEEEYRLHEKPVPSSIDPSWLQLFPTVWAERAGMGLANQVPPVVVELRSGASPVAVRQYPMSKEAREGIRPHIQRFLDLGVLVPCQSPWNTPLLPVKKPGTNDYRPVQDLREINKRVQDIHPTVPNPYNLLSSLPPSHTWYSVLDLKDAFFCLKLHPNSQPLFAFEWRDPEKGNTGQLTWTRLPQGFKNSPTLFDEALHRDLAPFRALNPQVVLLQYVDDLLVAAPTYRDCKEGTQKLLQELSKLGYRVSAKKAQLCQKEVTYLGYLLKEGKRWLTPARKATVMKIPPPTTPRQVREFLGTAGFCRLWIPGFASLAAPLYPLTKESIPFIWTEEHQKAFDRIKEALLSAPALALPDLTKPFTLYVDERAGVARGVLTQTLGPWRRPVAYLSKKLDPVASGWPTCLKAVAAVALLLKDADKLTLGQNVTVIASHSLESIVRQPPDRWMTNARMTHYQSLLLNERVSFAPPAVLNPATLLPVESEATPVHRCSEILAEETGTRRDLKDQPLPGVPAWYTDGSSFIAEGKRRAGAAIVDGKRTVWASSLPEGTSAQKAELVALTQALRLAEGKDINIYTDSRYAFATAHIHGAIYKQRGWLTSAGKDIKNKEEILALLEAIHLPKRVAIIHCPGHQKGNDPVATGNRRADEAAKQAALSTRVLAETTKPQELI

SEQ ID NO: 231 - protein, 672 aa, synthetic construct
LNLEEEYRLHEKPVPSSIDPSWLQLFPTVWAERAGMGLANQVPPVVVELRSGASPVAVRQYPMSKEAREGIRPHIQRFLDLGVLVPCQSPWNTPLLPVKKPGTNDYRPVQDLREINKRVQDIHPTVPNPYNLLSSLPPSHTWYSVLDLKDAFFCLKLHPNSQPLFAFEWRDPEKGNTGQLTWTRLPQGFKNSPTLFNEALHRDLAPFRALNPQVVLLQYVDDLLVAAPTYRDCKEGTQKLLQELSKLGYRVSAKKAQLCQKEVTYLGYLLKEGKRWLTPARKATVMKIPPPTTPRQVREFLGKAGFCRLFIPGFASLAAPLYPLTKPSIPFIWTEEHQKAFDRIKEALLSAPALALPDLTKPFTLYVDERAGVARGVLTQTLGPWRRPVAYLSKKLDPVASGWPTCLKAVAAVALLLKDADKLTLGQNVTVIASHSLESIVRQPPDRWMTNARMTHYQSLLLNERVSFAPPAVLNPATLLPVESEATPVHRCSEILAEETGTRRDLKDQPLPGVPAWYTDGSSFIAEGKRRAGAAIVDGKRTVWASSLPEGTSAQKAELVALTQALRLAEGKDINIYTDSRYAFATAHIHGAIYKQRGWLTSAGKDIKNKEEILALLEAIHLPKRVAIIHCPGHQKGNDPVATGNRRADEAAKQAALSTRVLAETTKPQELI

SEQ ID NO: 232 - protein, 667 aa, synthetic construct; identical to SEQ ID NO: 45

SEQ ID NO: 233 - protein, 667 aa, synthetic construct
TLQLDDEYRLYSPQVKPDQDIQSWLEQFPQAWAETAGMGLAKQVPPQVIQLKASATPVSVRQYPLSREAREGIWPHVQRLIQQGILVPVQSPWNTPLLPVRKPGTNDYRPVQDLREVNKRVQDIHPTVPNPYNLLSALPPERNWYTVLDLKDAFFCLRLHPTSQPLFAFEWRDPGTGRTGQLTWTRLPQGFKNSPTIFNEALHRDLANFRIQHPQVTLLQYVDDLLLAGATKQDCLEGTKALLLELSDLGYRASAKKAQICRREVTYLGYSLRGGQRWLTEARKKTVVQIPAPTTAKQVREFLGTAGFCRLWIPGFATLAAPLYPLTKEKGEFSWAPEHQKAFDAIKKALLSAPALALPDVTKPFTLYVDERKGVARGVLTQTLGPWRRPVAYLSKKLDPVASGWPVCLKAIAAVAILVKDADKLTLGQNITVIAPHALENIVRQPPDRWMTNARMTHYQSLLLTERVTFAPPAALNPATLLPEETDEPVTHDCHQLLIEETGVRKDLTDIPLTGEVLTWFTDGSSYVVEGKRMAGAAVVDGTHTIWASSLPEGTSAQKAELMALTQALRLAEGKSINIYTDSRYAFATAHVHGAIYKQRGLLTSAGREIKNKEEILSLLEALHLPKRLAIIHCPGHQKAKDLISRGNQMADRVAKQAAQAVNLLPI

SEQ ID NO: 234 - protein, 667 aa, synthetic construct
TLQLDDEYRLYSPQVKPDQDIQSWLEQFPQAWAETAGMGLAKQVPPQVIQLKASATPVSVRQYPLSREAREGIWPHVQRLIQQGILVPVQSPWNTPLLPVRKPGTNDYRPVQDLREVNKRVQDIHPTVPNPYNLLSALPPERNWYTVLDLKDAFFCLRLHPTSQPLFAFEWRDPGTGRTGQLTWTRLPQGFKNSPTIFDEALHRDLANFRIQHPQVTLLQYVDDLLLAGATKQDCLEGTKALLLELSDLGYRASAKKAQICRREVTYLGYSLRGGQRWLTEARKKTVVQIPAPTTAKQVREFLGKAGFCRLWIPGFATLAAPLYPLTKEKGEFSWAPEHQKAFDAIKKALLSAPALALPDVTKPFTLYVDERKGVARGVLTQTLGPWRRPVAYLSKKLDPVASGWPVCLKAIAAVAILVKDADKLTLGQNITVIAPHALENIVRQPPDRWMTNARMTHYQSLLLTERVTFAPPAALNPATLLPEETDEPVTHDCHQLLIEETGVRKDLTDIPLTGEVLTWFTDGSSYVVEGKRMAGAAVVDGTHTIWASSLPEGTSAQKAELMALTQALRLAEGKSINIYTDSRYAFATAHVHGAIYKQRGLLTSAGREIKNKEEILSLLEALHLPKRLAIIHCPGHQKAKDLISRGNQMADRVAKQAAQAVNLLPI

SEQ ID NO: 235 - protein, 667 aa, synthetic construct
TLQLDDEYRLYSPQVKPDQDIQSWLEQFPQAWAETAGMGLAKQVPPQVIQLKASATPVSVRQYPLSREAREGIWPHVQRLIQQGILVPVQSPWNTPLLPVRKPGTNDYRPVQDLREVNKRVQDIHPTVPNPYNLLSALPPERNWYTVLDLKDAFFCLRLHPTSQPLFAFEWRDPGTGRTGQLTWTRLPQGFKNSPTIFDEALHRDLANFRIQHPQVTLLQYVDDLLLAGATKQDCLEGTKALLLELSDLGYRASAKKAQICRREVTYLGYSLRGGQRWLTEARKKTVVQIPAPTTAKQVREFLGTAGFCRLFIPGFATLAAPLYPLTKEKGEFSWAPEHQKAFDAIKKALLSAPALALPDVTKPFTLYVDERKGVARGVLTQTLGPWRRPVAYLSKKLDPVASGWPVCLKAIAAVAILVKDADKLTLGQNITVIAPHALENIVRQPPDRWMTNARMTHYQSLLLTERVTFAPPAALNPATLLPEETDEPVTHDCHQLLIEETGVRKDLTDIPLTGEVLTWFTDGSSYVVEGKRMAGAAVVDGTHTIWASSLPEGTSAQKAELMALTQALRLAEGKSINIYTDSRYAFATAHVHGAIYKQRGLLTSAGREIKNKEEILSLLEALHLPKRLAIIHCPGHQKAKDLISRGNQMADRVAKQAAQAVNLLPI

SEQ ID NO: 236 - protein, 667 aa, synthetic construct
TLQLDDEYRLYSPQVKPDQDIQSWLEQFPQAWAETAGMGLAKQVPPQVIQLKASATPVSVRQYPLSREAREGIWPHVQRLIQQGILVPVQSPWNTPLLPVRKPGTNDYRPVQDLREVNKRVQDIHPTVPNPYNLLSALPPERNWYTVLDLKDAFFCLRLHPTSQPLFAFEWRDPGTGRTGQLTWTRLPQGFKNSPTIFDEALHRDLANFRIQHPQVTLLQYVDDLLLAGATKQDCLEGTKALLLELSDLGYRASAKKAQICRREVTYLGYSLRGGQRWLTEARKKTVVQIPAPTTAKQVREFLGTAGFCRLWIPGFATLAAPLYPLTKPKGEFSWAPEHQKAFDAIKKALLSAPALALPDVTKPFTLYVDERKGVARGVLTQTLGPWRRPVAYLSKKLDPVASGWPVCLKAIAAVAILVKDADKLTLGQNITVIAPHALENIVRQPPDRWMTNARMTHYQSLLLTERVTFAPPAALNPATLLPEETDEPVTHDCHQLLIEETGVRKDLTDIPLTGEVLTWFTDGSSYVVEGKRMAGAAVVDGTHTIWASSLPEGTSAQKAELMALTQALRLAEGKSINIYTDSRYAFATAHVHGAIYKQRGLLTSAGREIKNKEEILSLLEALHLPKRLAIIHCPGHQKAKDLISRGNQMADRVAKQAAQAVNLLPI

SEQ ID NO: 237 - protein, 667 aa, synthetic construct
TLQLDDEYRLYSPQVKPDQDIQSWLEQFPQAWAETAGMGLAKQVPPQVIQLKASATPVSVRQYPLSREAREGIWPHVQRLIQQGILVPVQSPWNTPLLPVRKPGTNDYRPVQDLREVNKRVQDIHPTVPNPYNLLSALPPERNWYTVLDLKDAFFCLRLHPTSQPLFAFEWRDPGTGRTGQLTWTRLPQGFKNSPTIFDEALHRDLANFRIQHPQVTLLQYVDDLLLAGATKQDCLEGTKALLLELSDLGYRASAKKAQICRREVTYLGYSLRGGQRWLTEARKKTVVQIPAPTTAKQVREFLGTAGFCRLWIPGFATLAAPLYPLTKEKGEFSWAPEHQKAFDAIKKALLSAPALALPDVTKPFTLYVDERKGVARGVLTQTLGPWRRPVAYLSKKLDPVASGWPVCLKAIAAVAILVKDADKLTLGQNITVIAPHALENIVRQPPDRWMTNARMTHYQSLLLTERVTFAPPAALNPATLLPEETDEPVTHDCHQLLIEETGVRKDLTDIPLTGEVLTWFTDGSSYVVEGKRMAGAAVVDGTHTIWASSLPEGTSAQKAELMALTQALRLAEGKSINIYTDSRYAFATAHVHGAIYKQRGWLTSAGREIKNKEEILSLLEALHLPKRLAIIHCPGHQKAKDLISRGNQMADRVAKQAAQAVNLLPI

SEQ ID NO: 238 - protein, 667 aa, synthetic construct
TLQLDDEYRLYSPQVKPDQDIQSWLEQFPQAWAETAGMGLAKQVPPQVIQLKASATPVSVRQYPLSREAREGIWPHVQRLIQQGILVPVQSPWNTPLLPVRKPGTNDYRPVQDLREVNKRVQDIHPTVPNPYNLLSALPPERNWYTVLDLKDAFFCLRLHPTSQPLFAFEWRDPGTGRTGQLTWTRLPQGFKNSPTIFNEALHRDLANFRIQHPQVTLLQYVDDLLLAGATKQDCLEGTKALLLELSDLGYRASAKKAQICRREVTYLGYSLRGGQRWLTEARKKTVVQIPAPTTAKQVREFLGKAGFCRLFIPGFATLAAPLYPLTKPKGEFSWAPEHQKAFDAIKKALLSAPALALPDVTKPFTLYVDERKGVARGVLTQTLGPWRRPVAYLSKKLDPVASGWPVCLKAIAAVAILVKDADKLTLGQNITVIAPHALENIVRQPPDRWMTNARMTHYQSLLLTERVTFAPPAALNPATLLPEETDEPVTHDCHQLLIEETGVRKDLTDIPLTGEVLTWFTDGSSYVVEGKRMAGAAVVDGTHTIWASSLPEGTSAQKAELMALTQALRLAEGKSINIYTDSRYAFATAHVHGAIYKQRGWLTSAGREIKNKEEILSLLEALHLPKRLAIIHCPGHQKAKDLISRGNQMADRVAKQAAQAVNLLPI

SEQ ID NO: 239 - protein, 513 aa, synthetic construct
AGQPTSREALYERIRSTSKEEVILEEMIRLGFWPAQGAVPHDPAEEIRRRGELERQLSELREKSRKLYNEKALIAEQRKQRLAESRRKQKETKARRERERQERAQKWAQRKAGEILFLGEDVSGGMSHKTCDAELIKREGVPAIASAEELARAMGITLKELRFLTYNRKVSRVTHYRRFLLPKKTGGLRLISAPMPRLKRAQAWALEHIFNKLSFEPAAHGFVAGRSIVSNARPHVGADVVVNLDLKDFFPTVSFPRVKGALRHLGYSESVATALALVCTEPEVDEVVLDGTTWYVARGERFLPQGSPCSPAITNLLCRRLDRRLHGLAQALGFVYTRYADDLTFSGRGEAAESKRVGKLLRGAADIVAHEGFVVHPDKTRVMRRGRRQEVTGVVVNDKTSVPRDELRKFRATLYQIEKDGPADKRWGNGGDVLAAVHGYACFVAMVDPSRGQPLLARARALLAKHGGPSKPPGGSGPRAPTPVQPTANAPEAPKPVAPATPAAPAKKGWKLF

SEQ ID NO: 240 - protein, 314 aa, synthetic construct
NILTTLREQLMTNNVIMPQEFERLEVRGSHAYKVYSIPKRKAGRRTIAHPSSKLKICQRHLNAILNPLLKVHDASYAYVKGRSIKDNALVHSHSAYMLKMDFQNFFNSITPTILRQCLIQNDILLSVNELEKLEQLIFWNPSKKRDGKLILSVGSPISPLISNAIMYPFDKIINDICTKHGINYTRYADDITFSTNIKNTLNKLPEIVEQLIIQTYAGRIIINKRKTVFSSKKHNRHVTGITLTTDSKISIGRSRKRYISSLVFKYINKNLDIDEINHMKGMLAFAYNIEPIYIHRLSHKYKVNIVEKILRGSN

SEQ ID NO: 241 - protein, 506 aa, synthetic construct
MISSSKHTLSQMNKVSNIVKEPELPDIYKEFKDITADTNTEKLPKPIKGLEFEVELTQENYRLPIRNYPLPPGKMQAMNDEINQGLKSGIIRESKAINACPVMFVPKKEGTLRMVVDYRPLNKYVKPNIYPLPLIEQLLAKIQGSTIFTKLDLKSAYHLIRVRKGDEHKLAFRCPRGVFEYLVMPYGIKTAPAHFQYFINTILGEAKESHVVCYMDDILIHSKSESEHVKHVKDVLQKLKNANLIINQAKCEFHQSQVKFLGYHISEKGFTPCQENIDKVLQWKQPKNQKELRQFLGQVNYLRKFIPKTSQLTHPLNKLLKKDVRWKWTPTQTQAIENIKQCLVSPPVLRHFDFSKKILLETDASDVAVGAVLSQKHDDDKYYPVGYYSAKMSKAQLNYSVSDKEMLAIIKSLKHWRHYLESTIEPFKILTDHRNLIGRITNESEPENKRLARWQLFLQDFNFEINYRPGSANHIADALSRIVDETEPIPKDSEDNSINFVNQISI

SEQ ID NO: 242 - protein, 505 aa, synthetic construct
ISSSKHTLSQMNKVSNIVKEPELPDIYKEFKDITADTNTEKLPKPIKGLEFEVELTQENYRLPIRNYPLTPVKMQAMNDEINQGLKSGIIRESKAINACPVIFVPRKEGTLRMVVDYKPLNKYVKPNIYPLPLIEQLLAKIQGSTIFTKLDLKSAYHQIRVRKGDEHKLAFRCPRGVFEYLVMPYGISTAPAHFQYCINTILGEAKESHVVCYMDDILIHSKSESEHVKHVKDVLQKLKNANLIINQAKCEFHQSQVKFIGYHISEKGLTPCQENIDKVLQWKQPKNRKELRQFLGSVNYLRKFIPKTSQLTHPLNKLLKKDVRWKWTPTQTQAIENIKQCLVSPPVLRHFDFSKKILLETDVSDVAVGAVLSQKHDDDKYYPVGYYSAKMSKAQLNYSVSDKEMLAIIKSLEHWRHYLESTIEPFKILTDHRNLIGRITNESEPENKRLARWQLFLQDFNFEINYRPGSANHIADALSRIVDETEPIPKDNEDNSINFVNQISI

SEQ ID NO: 243 - protein, 505 aa, synthetic construct
ISSSKHTLSQMNKVSNIVKEPELPDIYKEFKDITADTNTEKLPKPIKGLEFEVELTQENYRLPIRNYPLTPVKMQAMNDEINQGLKGGIIRESKAINACPVIFVPRKEGTLRMVVDYRPLNKYVKPNVYPLPLIEQLLAKIQGSTIFTKLDLKSAYHQIRVRKGDEHKLAFRCPRGVFEYLVMPYGISTAPAHFQYFINTILGEAKESHVVCYMDDILIHSKSESEHVKHVKDVLQKLKNANLIINQAKCEFHQSQVKFIGYHISEKGLTPCQENIDKVLQWKQPKNRKELRQFLGSVNYLRKFIPKTSQLTHPLNKLLKKDVRWKWTPTQTQAIENIKQCLVSPPVLRHFDFSKKILLETDVSDVAVGAVLSQKHDDDKYYPVGYYSAKMSKAQLNYSVSDKEMLAIIKSLEHWRHYLESTIEPFKILTDHRNLIGRITNESEPENKRLARWQLFLQDFNFEINYRPGSANHIADALSRIVDETEPIPKDNEDNSINFVNQISI

SEQ ID NO: 244 - protein, 505 aa, synthetic construct
ISSSKHTLSQMNKVSNIVKEPELPDIYKEFKDITADTNTEKLPKPIKGLEFEVELTQENYRLPIRNYPLTPVKMQAMNDEINQGLKGGIIRESKAINACPVIFVPRKEGTLRMVVDYRPLNKYVKPNVYPLPLIEQLLAKIQGSTIFTKLDLKSAYHQIRVRKGDEHKLAFRCPRGVFEYLVMPYGIKTAPAHFQYFINTILGEAKESHVVCYMDDILIHSKSESEHVKHVKDVLQKLKNANLIINQAKCEFHQSQVKFLGYHISEKGLTPCQENIDKVLQWKQPKNQKELRQFLGQVNYLRKFIPKTSQLTHPLNKLLKKDVRWKWTPTQTQAIENIKQCLVSPPVLRHFDFSKKILLETDVSDVAVGAVLSQKHDDDKYYPVGYYSAKMSKAQLNYSVSDKEMLAIIKSLEHWRHYLESTIEPFKILTDHRNLIGRITNESEPENKRLARWQLFLQDFNFEINYRPGSANHIADALSRIVDETEPIPKDNEDNSINFVNQISI

SEQ ID NO: 245 - protein, 505 aa, synthetic construct
ISSSKHTLSQMNKVSNIVKEPELPDIYKEFKDITADTNTEKLPKPIKGLEFEVELTQENYRLPIRNYPLTPVKMQAMNDEINQGLKSGIIRESKAINACPVIFVPRKEGTLRMVVDYRPLNKYVKPNIYPLPLIEQLLAKIQGSTIFTKLDLKSAYHQIRVRKGDEHKLAFRCPRGVFEYLVMPYGIKTAPAHFQYCINTILGEAKESHVVCYMDDILIHSKSESEHVKHVKDVLQKLKNANLIINQAKCEFHQSQVKFLGYHISEKGLTPCQENIDKVLQWKQPKNQKELRQFLGQVNYLRKFIPKTSQLTHPLNKLLKKDVRWKWTPTQTQAIENIKQCLVSPPVLRHFDFSKKILLETDVSDVAVGAVLSQKHDDDKYYPVGYYSAKMSKAQLNYSVSDKEMLAIIKSLEHWRHYLESTIEPFKILTDHRNLIGRITNESEPENKRLARWQLFLQDFNFEINYRPGSANHIADALSRIVDETEPIPKDNEDNSINFVNQISI

SEQ ID NO: 246-255 - skipped (10 entries)

SEQ ID NO: 256 - protein, 190 aa, synthetic construct
GGCCCAGACTGAGCACGTGAGTTTTAGAGCTAGAAATAGCAAGTTAAAATAAGGCTAGTCCGTTATCAACTTGAAAAAGTGGCACCGAGTCGGTGCGCCCTCTGGAGGAAGCAGGGCTTCCCGTGCTCAGTCTGTCTCTCTCTTGACGCGGTTCTATCTAGTTACGCGTTAAACCAACTAGAAATTTTTT

SEQ ID NO: 257 - DNA, 201 bp, synthetic construct
  source 1..201: mol_type = other DNA
ggcccagactgagcacgtgagtttaagagctaagctggaaacagcatagcaagtttaaataaggctagtccgttatcaactcgagagagtggcaccgagtcggtgctgccctctggaggaagcagggcttcccgtgctcagtctgtctctctcttgacgcggttctatctagttacgcgttaaaccaactagaaatttttt

SEQ ID NO: 258 - DNA, 201 bp, synthetic construct
  source 1..201: mol_type = other DNA
ggcccagactgagcacgtgagtttaagagctaagctggaaacagcatagcaagtttaaataaggctagtccgttatcaactggaaacagtggcaccgagtcggtgctgccctctggaggaagcagggcttcccgtgctcagtctgtctctctcttgacgcggttctatctagttacgcgttaaaccaactagaaatttttt

SEQ ID NO: 259 - DNA, 201 bp, synthetic construct
  source 1..201: mol_type = other DNA
ggcccagactgagcacgtgagtttaagagctaagctggaaacagcatagcaagtttaaataaggctagtccgttatcagctggaaacagcggcaccgagtcggtgctgccctctggaggaagcagggcttcccgtgctcagtctgtctctctcttgacgcggttctatctagttacgcgttaaaccaactagaaatttttt

SEQ ID NO: 260 - DNA, 201 bp, synthetic construct
  source 1..201: mol_type = other DNA
ggcccagactgagcacgtgagtttaagagctaagctggaaacagcatagcaagtttaaataaggctagtccgttatcaactcgaaagagtggcaccgagtcggtgctgccctctggaggaagcagggcttcccgtgctcagtctgtctctctcttgacgcggttctatctagttacgcgttaaaccaactagaaatttttt

SEQ ID NO: 261 - DNA, 201 bp, synthetic construct
  source 1..201: mol_type = other DNA
ggcccagactgagcacgtgagtttaagagctaagctggaaacagcatagcaagtttaaataaggctagtccgttatcaacgtgaaaacgtgactccgagtcggagttgccctctggaggaagcagggcttcccgtgctcagtctgtctctctcttgacgcggttctatctagttacgcgttaaaccaactagaaatttttt

SEQ ID NO: 262 - DNA, 201 bp, synthetic construct
  source 1..201: mol_type = other DNA
ggcccagactgagcacgtgagtttaagagctaagctggaaacagcatagcaagtttaaataaggctagtccgttatcaactcgaaagagtgcaaccgagtcggttgtgccctctggaggaagcagggcttcccgtgctcagtctgtctctctcttgacgcggttctatctagttacgcgttaaaccaactagaaatttttt

SEQ ID NO: 263 - DNA, 201 bp, synthetic construct
  source 1..201: mol_type = other DNA
ggcccagactgagcacgtgagtttaagagctaagctggaaacagcatagcaagtttaaataaggctagtccgttatcaacttgaaaaagtggcaccgagtcggtgctgccctctggaggaagcagggcttcccgtgctcagtctgtctctctcttgacgcggttctatctagttacgcgttaaaccaactagaaatttttt

SEQ ID NO: 264 - DNA, 203 bp, synthetic construct
  source 1..203: mol_type = other DNA
gtcatcttagtcattacctggtttaagagctaagctggaaacagcatagcaagtttaaataaggctagtccgttatcaactcgagagagtggcaccgagtcggtgctatgggaactcagtttatatgagttagtaatgactaagatgtcatctctttgacgcggttctatctagttacgcgttaaaccaactagaaatttttt

SEQ ID NO: 265 - DNA, 203 bp, synthetic construct
  source 1..203: mol_type = other DNA
gtcatcttagtcattacctggtttaagagctaagctggaaacagcatagcaagtttaaataaggctagtccgttatcaactggaaacagtggcaccgagtcggtgctatgggaactcagtttatatgagttagtaatgactaagatgtcatctctttgacgcggttctatctagttacgcgttaaaccaactagaaatttttt

SEQ ID NO: 266 - DNA, 203 bp, synthetic construct
  source 1..203: mol_type = other DNA
gtcatcttagtcattacctggtttaagagctaagctggaaacagcatagcaagtttaaataaggctagtccgttatcagctggaaacagcggcaccgagtcggtgctatgggaactcagtttatatgagttagtaatgactaagatgtcatctctttgacgcggttctatctagttacgcgttaaaccaactagaaatttttt

SEQ ID NO: 267 - DNA, 203 bp, synthetic construct
  source 1..203: mol_type = other DNA
gtcatcttagtcattacctggtttaagagctaagctggaaacagcatagcaagtttaaataaggctagtccgttatcaactcgaaagagtggcaccgagtcggtgctatgggaactcagtttatatgagttagtaatgactaagatgtcatctctttgacgcggttctatctagttacgcgttaaaccaactagaaatttttt

SEQ ID NO: 268 - DNA, 203 bp, synthetic construct
  source 1..203: mol_type = other DNA
gtcatcttagtcattacctggtttaagagctaagctggaaacagcatagcaagtttaaataaggctagtccgttatcaacgtgaaaacgtgactccgagtcggagttatgggaactcagtttatatgagttagtaatgactaagatgtcatctctttgacgcggttctatctagttacgcgttaaaccaactagaaatttttt

SEQ ID NO: 269 - DNA, 203 bp, synthetic construct
  source 1..203: mol_type = other DNA
gtcatcttagtcattacctggtttaagagctaagctggaaacagcatagcaagtttaaataaggctagtccgttatcaactcgaaagagtgcaaccgagtcggttgtatgggaactcagtttatatgagttagtaatgactaagatgtcatctctttgacgcggttctatctagttacgcgttaaaccaactagaaatttttt

SEQ ID NO: 270 - DNA, 203 bp, synthetic construct
  source 1..203: mol_type = other DNA
gtcatcttagtcattacctggtttaagagctaagctggaaacagcatagcaagtttaaataaggctagtccgttatcaacttgaaaaagtggcaccgagtcggtgctatgggaactcagtttatatgagttagtaatgactaagatgtcatctctttgacgcggttctatctagttacgcgttaaaccaactagaaatttttt

SEQ ID NO: 271 - DNA, 192 bp, synthetic construct
  source 1..192: mol_type = other DNA
gcattttcaggaggaagcgagttttagagctagaaatagcaagttaaaataaggctagtccgttatcaacttgaaaaagtggcaccgagtcggtgctacgaaggaaatgactcaaatatgccttcctcctgaaaataactctctttgacgcggttctatctagttacgcgttaaaccaactagaaatttttt

SEQ ID NO: 272 - DNA, 203 bp, synthetic construct
  source 1..203: mol_type = other DNA
gcattttcaggaggaagcgagtttaagagctaagctggaaacagcatagcaagtttaaataaggctagtccgttatcaactcgagagagtggcaccgagtcggtgcttacgaaggaaatgactcaaatatgccttcctcctgaaaataactctctttgacgcggttctatctagttacgcgttaaaccaactagaaatttttt

SEQ ID NO: 273 - DNA, 203 bp, synthetic construct
  source 1..203: mol_type = other DNA
gcattttcaggaggaagcgagtttaagagctaagctggaaacagcatagcaagtttaaataaggctagtccgttatcaactggaaacagtggcaccgagtcggtgcttacgaaggaaatgactcaaatatgccttcctcctgaaaataactctctttgacgcggttctatctagttacgcgttaaaccaactagaaatttttt

SEQ ID NO: 274 - DNA, 203 bp, synthetic construct
  source 1..203: mol_type = other DNA
gcattttcaggaggaagcgagtttaagagctaagctggaaacagcatagcaagtttaaataaggctagtccgttatcagctggaaacagcggcaccgagtcggtgcttacgaaggaaatgactcaaatatgccttcctcctgaaaataactctctttgacgcggttctatctagttacgcgttaaaccaactagaaatttttt

SEQ ID NO: 275 - DNA, 203 bp, synthetic construct
  source 1..203: mol_type = other DNA
gcattttcaggaggaagcgagtttaagagctaagctggaaacagcatagcaagtttaaataaggctagtccgttatcaactcgaaagagtggcaccgagtcggtgcttacgaaggaaatgactcaaatatgccttcctcctgaaaataactctctttgacgcggttctatctagttacgcgttaaaccaactagaaatttttt

SEQ ID NO: 276 - DNA, 203 bp, synthetic construct
  source 1..203: mol_type = other DNA
gcattttcaggaggaagcgagtttaagagctaagctggaaacagcatagcaagtttaaataaggctagtccgttatcaacgtgaaaacgtgactccgagtcggagtttacgaaggaaatgactcaaatatgccttcctcctgaaaataactctctttgacgcggttctatctagttacgcgttaaaccaactagaaatttttt

SEQ ID NO: 277 - DNA, 203 bp, synthetic construct
  source 1..203: mol_type = other DNA
gcattttcaggaggaagcgagtttaagagctaagctggaaacagcatagcaagtttaaataaggctagtccgttatcaactcgaaagagtgcaaccgagtcggttgttacgaaggaaatgactcaaatatgccttcctcctgaaaataactctctttgacgcggttctatctagttacgcgttaaaccaactagaaatttttt

SEQ ID NO: 278 - DNA, 203 bp, synthetic construct
  source 1..203: mol_type = other DNA
gcattttcaggaggaagcgagtttaagagctaagctggaaacagcatagcaagtttaaataaggctagtccgttatcaacttgaaaaagtggcaccgagtcggtgcttacgaaggaaatgactcaaatatgccttcctcctgaaaataactctctttgacgcggttctatctagttacgcgttaaaccaactagaaatttttt

SEQ ID NO: 279 - DNA, 182 bp, synthetic construct
  source 1..182: mol_type = other DNA
gcattttcaggaggaagcgagttttagagctagaaatagcaagttaaaataaggctagtccgttatcaacttgaaaaagtggcaccgagtcggtgctgtctgaagcaatcgcttcctcctgaaaataactctctttgacgcggttctatctagttacgcgttaaaccaactagaaatttttt

SEQ ID NO: 280 - DNA, 193 bp, synthetic construct
  source 1..193: mol_type = other DNA
gcattttcaggaggaagcgagtttaagagctaagctggaaacagcatagcaagtttaaataaggctagtccgttatcaactcgagagagtggcaccgagtcggtgcttgtctgaagcaatcgcttcctcctgaaaataactctctttgacgcggttctatctagttacgcgttaaaccaactagaaatttttt

SEQ ID NO: 281 - DNA, 193 bp, synthetic construct
  source 1..193: mol_type = other DNA
gcattttcaggaggaagcgagtttaagagctaagctggaaacagcatagcaagtttaaataaggctagtccgttatcaactggaaacagtggcaccgagtcggtgcttgtctgaagcaatcgcttcctcctgaaaataactctctttgacgcggttctatctagttacgcgttaaaccaactagaaatttttt

SEQ ID NO: 282 - DNA, 193 bp, synthetic construct
  source 1..193: mol_type = other DNA
gcattttcaggaggaagcgagtttaagagctaagctggaaacagcatagcaagtttaaataaggctagtccgttatcagctggaaacagcggcaccgagtcggtgcttgtctgaagcaatcgcttcctcctgaaaataactctctttgacgcggttctatctagttacgcgttaaaccaactagaaatttttt

SEQ ID NO: 283 - DNA, 193 bp, synthetic construct
  source 1..193: mol_type = other DNA
gcattttcaggaggaagcgagtttaagagctaagctggaaacagcatagcaagtttaaataaggctagtccgttatcaactcgaaagagtggcaccgagtcggtgcttgtctgaagcaatcgcttcctcctgaaaataactctctttgacgcggttctatctagttacgcgttaaaccaactagaaatttttt

SEQ ID NO: 284 - DNA, 193 bp, synthetic construct
  source 1..193: mol_type = other DNA
gcattttcaggaggaagcgagtttaagagctaagctggaaacagcatagcaagtttaaataaggctagtccgttatcaacgtgaaaacgtgactccgagtcggagtttgtctgaagcaatcgcttcctcctgaaaataactctctttgacgcggttctatctagttacgcgttaaaccaactagaaatttttt

SEQ ID NO: 285 - DNA, 193 bp, synthetic construct
  source 1..193: mol_type = other DNA
gcattttcaggaggaagcgagtttaagagctaagctggaaacagcatagcaagtttaaataaggctagtccgttatcaactcgaaagagtgcaaccgagtcggttgttgtctgaagcaatcgcttcctcctgaaaataactctctttgacgcggttctatctagttacgcgttaaaccaactagaaatttttt

SEQ ID NO: 286 - DNA, 193 bp, synthetic construct
  source 1..193: mol_type = other DNA
gcattttcaggaggaagcgagtttaagagctaagctggaaacagcatagcaagtttaaataaggctagtccgttatcaacttgaaaaagtggcaccgagtcggtgcttgtctgaagcaatcgcttcctcctgaaaataactctctttgacgcggttctatctagttacgcgttaaaccaactagaaatttttt

SEQ ID NO: 287 - DNA, 187 bp, synthetic construct
  source 1..187: mol_type = other DNA
gattcctggtgccagaaacagttttagagctagaaatagcaagttaaaataaggctagtccgttatcaacttgaaaaagtggcaccgagtcggtgctgctaaggactagttctgcccttctggcaccaggacctcttctttgacgcggttctatctagttacgcgttaaaccaactagaaatttttt

SEQ ID NO: 288 - DNA, 198 bp, synthetic construct
  source 1..198: mol_type = other DNA
gattcctggtgccagaaacagtttaagagctaagctggaaacagcatagcaagtttaaataaggctagtccgttatcaactcgagagagtggcaccgagtcggtgcttgctaaggactagttctgcccttctggcaccaggacctcttctttgacgcggttctatctagttacgcgttaaaccaactagaaatttttt

SEQ ID NO: 289 - DNA, 198 bp, synthetic construct
  source 1..198: mol_type = other DNA
gattcctggtgccagaaacagtttaagagctaagctggaaacagcatagcaagtttaaataaggctagtccgttatcaactggaaacagtggcaccgagtcggtgcttgctaaggactagttctgcccttctggcaccaggacctcttctttgacgcggttctatctagttacgcgttaaaccaactagaaatttttt

SEQ ID NO: 290 - DNA, 198 bp, synthetic construct
  source 1..198: mol_type = other DNA
gattcctggtgccagaaacagtttaagagctaagctggaaacagcatagcaagtttaaataaggctagtccgttatcagctggaaacagcggcaccgagtcggtgcttgctaaggactagttctgcccttctggcaccaggacctcttctttgacgcggttctatctagttacgcgttaaaccaactagaaatttttt

SEQ ID NO: 291 - DNA, 198 bp, synthetic construct
  source 1..198: mol_type = other DNA
gattcctggtgccagaaacagtttaagagctaagctggaaacagcatagcaagtttaaataaggctagtccgttatcaactcgaaagagtggcaccgagtcggtgcttgctaaggactagttctgcccttctggcaccaggacctcttctttgacgcggttctatctagttacgcgttaaaccaactagaaatttttt

SEQ ID NO: 292 - DNA, 198 bp, synthetic construct
  source 1..198: mol_type = other DNA
gattcctggtgccagaaacagtttaagagctaagctggaaacagcatagcaagtttaaataaggctagtccgttatcaacgtgaaaacgtgactccgagtcggagtttgctaaggactagttctgcccttctggcaccaggacctcttctttgacgcggttctatctagttacgcgttaaaccaactagaaatttttt

SEQ ID NO: 293 - DNA, 198 bp, synthetic construct
  source 1..198: mol_type = other DNA
gattcctggtgccagaaacagtttaagagctaagctggaaacagcatagcaagtttaaataaggctagtccgttatcaactcgaaagagtgcaaccgagtcggttgttgctaaggactagttctgcccttctggcaccaggacctcttctttgacgcggttctatctagttacgcgttaaaccaactagaaatttttt

SEQ ID NO: 294 - DNA, 198 bp, synthetic construct
  source 1..198: mol_type = other DNA
gattcctggtgccagaaacagtttaagagctaagctggaaacagcatagcaagtttaaataaggctagtccgttatcaacttgaaaaagtggcaccgagtcggtgcttgctaaggactagttctgcccttctggcaccaggacctcttctttgacgcggttctatctagttacgcgttaaaccaactagaaatttttt

SEQ ID NO: 295 - DNA, 177 bp, synthetic construct
  source 1..177: mol_type = other DNA
gattcctggtgccagaaacagttttagagctagaaatagcaagttaaaataaggctagtccgttatcaacttgaaaaagtggcaccgagtcggtgcgtcaccactgtttctggcaccaggacctcttctttgacgcggttctatctagttacgcgttaaaccaactagaaatttttt

SEQ ID NO: 296 - DNA, 188 bp, synthetic construct
  source 1..188: mol_type = other DNA
gattcctggtgccagaaacagtttaagagctaagctggaaacagcatagcaagtttaaataaggctagtccgttatcaactcgagagagtggcaccgagtcggtgctgtcaccactgtttctggcaccaggacctcttctttgacgcggttctatctagttacgcgttaaaccaactagaaatttttt

SEQ ID NO: 297 - DNA, 188 bp, synthetic construct
  source 1..188: mol_type = other DNA
gattcctggtgccagaaacagtttaagagctaagctggaaacagcatagcaagtttaaataaggctagtccgttatcaactggaaacagtggcaccgagtcggtgctgtcaccactgtttctggcaccaggacctcttctttgacgcggttctatctagttacgcgttaaaccaactagaaatttttt

SEQ ID NO: 298 - DNA, 188 bp, synthetic construct
  source 1..188: mol_type = other DNA
gattcctggtgccagaaacagtttaagagctaagctggaaacagcatagcaagtttaaataaggctagtccgttatcagctggaaacagcggcaccgagtcggtgctgtcaccactgtttctggcaccaggacctcttctttgacgcggttctatctagttacgcgttaaaccaactagaaatttttt

SEQ ID NO: 299 - DNA, 188 bp, synthetic construct
  source 1..188: mol_type = other DNA
gattcctggtgccagaaacagtttaagagctaagctggaaacagcatagcaagtttaaataaggctagtccgttatcaactcgaaagagtggcaccgagtcggtgctgtcaccactgtttctggcaccaggacctcttctttgacgcggttctatctagttacgcgttaaaccaactagaaatttttt

SEQ ID NO: 300 - DNA, 188 bp, synthetic construct
  source 1..188: mol_type = other DNA
gattcctggtgccagaaacagtttaagagctaagctggaaacagcatagcaagtttaaataaggctagtccgttatcaacgtgaaaacgtgactccgagtcggagttgtcaccactgtttctggcaccaggacctcttctttgacgcggttctatctagttacgcgttaaaccaactagaaatttttt

SEQ ID NO: 301 - DNA, 188 bp, synthetic construct
  source 1..188: mol_type = other DNA
gattcctggtgccagaaacagtttaagagctaagctggaaacagcatagcaagtttaaataaggctagtccgttatcaactcgaaagagtgcaaccgagtcggttgtgtcaccactgtttctggcaccaggacctcttctttgacgcggttctatctagttacgcgttaaaccaactagaaatttttt

SEQ ID NO: 302 - DNA, 188 bp, synthetic construct
  source 1..188: mol_type = other DNA
gattcctggtgccagaaacagtttaagagctaagctggaaacagcatagcaagtttaaataaggctagtccgttatcaacttgaaaaagtggcaccgagtcggtgctgtcaccactgtttctggcaccaggacctcttctttgacgcggttctatctagttacgcgttaaaccaactagaaatttttt

SEQ ID NO: 303 - DNA, 192 bp, synthetic construct
  source 1..192: mol_type = other DNA
ggaatcccttctgcagcaccgttttagagctagaaatagcaagttaaaataaggctagtccgttatcaacttgaaaaagtggcaccgagtcggtgctagtgcttgagaccgccagaagctcgggctgcagaagggacaatcactttgacgcggttctatctagttacgcgttaaaccaactagaaatttttt

SEQ ID NO: 304 - DNA, 203 bp, synthetic construct
  source 1..203: mol_type = other DNA
ggaatcccttctgcagcaccgtttaagagctaagctggaaacagcatagcaagtttaaataaggctagtccgttatcaactcgagagagtggcaccgagtcggtgcttagtgcttgagaccgccagaagctcgggctgcagaagggacaatcactttgacgcggttctatctagttacgcgttaaaccaactagaaatttttt

SEQ ID NO: 305 - DNA, 203 bp, synthetic construct
  source 1..203: mol_type = other DNA
ggaatcccttctgcagcaccgtttaagagctaagctggaaacagcatagcaagtttaaataaggctagtccgttatcaactggaaacagtggcaccgagtcggtgcttagtgcttgagaccgccagaagctcgggctgcagaagggacaatcactttgacgcggttctatctagttacgcgttaaaccaactagaaatttttt

SEQ ID NO: 306 - DNA, 203 bp, synthetic construct
  source 1..203: mol_type = other DNA
ggaatcccttctgcagcaccgtttaagagctaagctggaaacagcatagcaagtttaaataaggctagtccgttatcagctggaaacagcggcaccgagtcggtgcttagtgcttgagaccgccagaagctcgggctgcagaagggacaatcactttgacgcggttctatctagttacgcgttaaaccaactagaaatttttt

SEQ ID NO: 307 - DNA, 203 bp, synthetic construct
  source 1..203: mol_type = other DNA
ggaatcccttctgcagcaccgtttaagagctaagctggaaacagcatagcaagtttaaataaggctagtccgttatcaactcgaaagagtggcaccgagtcggtgcttagtgcttgagaccgccagaagctcgggctgcagaagggacaatcactttgacgcggttctatctagttacgcgttaaaccaactagaaatttttt

SEQ ID NO: 308 - DNA, 203 bp, synthetic construct
  source 1..203: mol_type = other DNA
ggaatcccttctgcagcaccgtttaagagctaagctggaaacagcatagcaagtttaaataaggctagtccgttatcaacgtgaaaacgtgactccgagtcggagtttagtgcttgagaccgccagaagctcgggctgcagaagggacaatcactttgacgcggttctatctagttacgcgttaaaccaactagaaatttttt

SEQ ID NO: 309 - DNA, 203 bp, synthetic construct
  source 1..203: mol_type = other DNA
ggaatcccttctgcagcaccgtttaagagctaagctggaaacagcatagcaagtttaaataaggctagtccgttatcaactcgaaagagtgcaaccgagtcggttgttagtgcttgagaccgccagaagctcgggctgcagaagggacaatcactttgacgcggttctatctagttacgcgttaaaccaactagaaatttttt

SEQ ID NO: 310 - DNA, 203 bp, synthetic construct
  source 1..203: mol_type = other DNA
ggaatcccttctgcagcaccgtttaagagctaagctggaaacagcatagcaagtttaaataaggctagtccgttatcaacttgaaaaagtggcaccgagtcggtgcttagtgcttgagaccgccagaagctcgggctgcagaagggacaatcactttgacgcggttctatctagttacgcgttaaaccaactagaaatttttt

SEQ ID NO: 311 - DNA, 193 bp, synthetic construct
  source 1..193: mol_type = other DNA
ggaatcccttctgcagcaccgtttaagagctaagctggaaacagcatagcaagtttaaataaggctagtccgttatcaactcgagagagtggcaccgagtcggtgctggaaaagcgatcaaggtgctgcagaagggacaatcactttgacgcggttctatctagttacgcgttaaaccaactagaaatttttt

SEQ ID NO: 312 - DNA, 193 bp, synthetic construct
  source 1..193: mol_type = other DNA
ggaatcccttctgcagcaccgtttaagagctaagctggaaacagcatagcaagtttaaataaggctagtccgttatcaactggaaacagtggcaccgagtcggtgctggaaaagcgatcaaggtgctgcagaagggacaatcactttgacgcggttctatctagttacgcgttaaaccaactagaaatttttt

SEQ ID NO: 313 - DNA, 193 bp, synthetic construct
  source 1..193: mol_type = other DNA
ggaatcccttctgcagcaccgtttaagagctaagctggaaacagcatagcaagtttaaataaggctagtccgttatcagctggaaacagcggcaccgagtcggtgctggaaaagcgatcaaggtgctgcagaagggacaatcactttgacgcggttctatctagttacgcgttaaaccaactagaaatttttt

SEQ ID NO: 314 - DNA, 193 bp, synthetic construct
  source 1..193: mol_type = other DNA
ggaatcccttctgcagcaccgtttaagagctaagctggaaacagcatagcaagtttaaataaggctagtccgttatcaactcgaaagagtggcaccgagtcggtgctggaaaagcgatcaaggtgctgcagaagggacaatcactttgacgcggttctatctagttacgcgttaaaccaactagaaatttttt

SEQ ID NO: 315 - DNA, 193 bp, synthetic construct
  source 1..193: mol_type = other DNA
ggaatcccttctgcagcaccgtttaagagctaagctggaaacagcatagcaagtttaaataaggctagtccgttatcaacgtgaaaacgtgactccgagtcggagttggaaaagcgatcaaggtgctgcagaagggacaatcactttgacgcggttctatctagttacgcgttaaaccaactagaaatttttt

SEQ ID NO: 316 - DNA, 193 bp, synthetic construct
  source 1..193: mol_type = other DNA
ggaatcccttctgcagcaccgtttaagagctaagctggaaacagcatagcaagtttaaataaggctagtccgttatcaactcgaaagagtgcaaccgagtcggttgtggaaaagcgatcaaggtgctgcagaagggacaatcactttgacgcggttctatctagttacgcgttaaaccaactagaaatttttt

SEQ ID NO: 317 - DNA, 193 bp, synthetic construct
  source 1..193: mol_type = other DNA
ggaatcccttctgcagcaccgtttaagagctaagctggaaacagcatagcaagtttaaataaggctagtccgttatcaacttgaaaaagtggcaccgagtcggtgctggaaaagcgatcaaggtgctgcagaagggacaatcactttgacgcggttctatctagttacgcgttaaaccaactagaaatttttt

SEQ ID NO: 318 - DNA, 192 bp, synthetic construct
  source 1..192: mol_type = other DNA
gagtccgagcagaagaagaagttttagagctagaaatagcaagttaaaataaggctagtccgttatcaacttgaaaaagtggcaccgagtcggtgctcgtggcaatgcgccaccggttgatgttcttctgctcggaaacaatctttgacgcggttctatctagttacgcgttaaaccaactagaaatttttt

SEQ ID NO: 319 - DNA, 203 bp, synthetic construct
  source 1..203: mol_type = other DNA
gagtccgagcagaagaagaagtttaagagctaagctggaaacagcatagcaagtttaaataaggctagtccgttatcaactcgagagagtggcaccgagtcggtgcttcgtggcaatgcgccaccggttgatgttcttctgctcggaaacaatctttgacgcggttctatctagttacgcgttaaaccaactagaaatttttt

SEQ ID NO: 320 - DNA, 203 bp, synthetic construct
  source 1..203: mol_type = other DNA
gagtccgagcagaagaagaagtttaagagctaagctggaaacagcatagcaagtttaaataaggctagtccgttatcaactggaaacagtggcaccgagtcggtgcttcgtggcaatgcgccaccggttgatgttcttctgctcggaaacaatctttgacgcggttctatctagttacgcgttaaaccaactagaaatttttt

SEQ ID NO: 321 - DNA, 203 bp, synthetic construct
  source 1..203: mol_type = other DNA
gagtccgagcagaagaagaagtttaagagctaagctggaaacagcatagcaagtttaaataaggctagtccgttatcagctggaaacagcggcaccgagtcggtgcttcgtggcaatgcgccaccggttgatgttcttctgctcggaaacaatctttgacgcggttctatctagttacgcgttaaaccaactagaaatttttt

SEQ ID NO: 322 - DNA, 203 bp, synthetic construct
  source 1..203: mol_type = other DNA
gagtccgagcagaagaagaagtttaagagctaagctggaaacagcatagcaagtttaaataaggctagtccgttatcaactcgaaagagtggcaccgagtcggtgcttcgtggcaatgcgccaccggttgatgttcttctgctcggaaacaatctttgacgcggttctatctagttacgcgttaaaccaactagaaatttttt

SEQ ID NO: 323 - DNA, 203 bp, synthetic construct
  source 1..203: mol_type = other DNA
gagtccgagcagaagaagaagtttaagagctaagctggaaacagcatagcaagtttaaataaggctagtccgttatcaacgtgaaaacgtgactccgagtcggagtttcgtggcaatgcgccaccggttgatgttcttctgctcggaaacaatctttgacgcggttctatctagttacgcgttaaaccaactagaaatttttt

SEQ ID NO: 324 - DNA, 203 bp, synthetic construct
  source 1..203: mol_type = other DNA
gagtccgagcagaagaagaagtttaagagctaagctggaaacagcatagcaagtttaaataaggctagtccgttatcaactcgaaagagtgcaaccgagtcggttgttcgtggcaatgcgccaccggttgatgttcttctgctcggaaacaatctttgacgcggttctatctagttacgcgttaaaccaactagaaatttttt

SEQ ID NO: 325 - DNA, 203 bp, synthetic construct
  source 1..203: mol_type = other DNA
gagtccgagcagaagaagaagtttaagagctaagctggaaacagcatagcaagtttaaataaggctagtccgttatcaacttgaaaaagtggcaccgagtcggtgcttcgtggcaatgcgccaccggttgatgttcttctgctcggaaacaatctttgacgcggttctatctagttacgcgttaaaccaactagaaatttttt

SEQ ID NO: 326 - DNA, 182 bp, synthetic construct
  source 1..182: mol_type = other DNA
gagtccgagcagaagaagaagttttagagctagaaatagcaagttaaaataaggctagtccgttatcaacttgaaaaagtggcaccgagtcggtgcgtgatgggagcacttcttcttctgctcggaaacaatctttgacgcggttctatctagttacgcgttaaaccaactagaaatttttt

SEQ ID NO: 327 - DNA, 193 bp, synthetic construct
  source 1..193: mol_type = other DNA
gagtccgagcagaagaagaagtttaagagctaagctggaaacagcatagcaagtttaaataaggctagtccgttatcaactcgagagagtggcaccgagtcggtgctgtgatgggagcacttcttcttctgctcggaaacaatctttgacgcggttctatctagttacgcgttaaaccaactagaaatttttt

SEQ ID NO: 328 - DNA, 193 bp, synthetic construct
  source 1..193: mol_type = other DNA
gagtccgagcagaagaagaagtttaagagctaagctggaaacagcatagcaagtttaaataaggctagtccgttatcaactggaaacagtggcaccgagtcggtgctgtgatgggagcacttcttcttctgctcggaaacaatctttgacgcggttctatctagttacgcgttaaaccaactagaaatttttt

SEQ ID NO: 329 - DNA, 193 bp, synthetic construct
  source 1..193: mol_type = other DNA
gagtccgagcagaagaagaagtttaagagctaagctggaaacagcatagcaagtttaaataaggctagtccgttatcagctggaaacagcggcaccgagtcggtgctgtgatgggagcacttcttcttctgctcggaaacaatctttgacgcggttctatctagttacgcgttaaaccaactagaaatttttt

SEQ ID NO: 330 - DNA, 193 bp, synthetic construct
  source 1..193: mol_type = other DNA
gagtccgagcagaagaagaagtttaagagctaagctggaaacagcatagcaagtttaaataaggctagtccgttatcaactcgaaagagtggcaccgagtcggtgctgtgatgggagcacttcttcttctgctcggaaacaatctttgacgcggttctatctagttacgcgttaaaccaactagaaatttttt

SEQ ID NO: 331 - DNA, 193 bp, synthetic construct
  source 1..193: mol_type = other DNA
gagtccgagcagaagaagaagtttaagagctaagctggaaacagcatagcaagtttaaataaggctagtccgttatcaacgtgaaaacgtgactccgagtcggagttgtgatgggagcacttcttcttctgctcggaaacaatctttgacgcggttctatctagttacgcgttaaaccaactagaaatttttt

SEQ ID NO: 332 - DNA, 193 bp, synthetic construct
  source 1..193: mol_type = other DNA
gagtccgagcagaagaagaagtttaagagctaagctggaaacagcatagcaagtttaaataaggctagtccgttatcaactcgaaagagtgcaaccgagtcggttgtgtgatgggagcacttcttcttctgctcggaaacaatctttgacgcggttctatctagttacgcgttaaaccaactagaaatttttt

SEQ ID NO: 333 - DNA, 193 bp, synthetic construct
  source 1..193: mol_type = other DNA
gagtccgagcagaagaagaagtttaagagctaagctggaaacagcatagcaagtttaaataaggctagtccgttatcaacttgaaaaagtggcaccgagtcggtgctgtgatgggagcacttcttcttctgctcggaaacaatctttgacgcggttctatctagttacgcgttaaaccaactagaaatttttt

SEQ ID NO: 334 - DNA, 213 bp, synthetic construct
  source 1..213: mol_type = other DNA
gtcatcttagtcattacctggttttagagctagaaatagcaagttaaaataaggctagtccgttatcaacttgaaaaagtggcaccgagtcggtgctgagttacaacgaacacctcagcttatcgtcgtcatccttgtaatcgtaatgactaagatgtcatctctttgacgcggttctatctagttacgcgttaaaccaactagaaatttttt

SEQ ID NO: 335 - DNA, 224 bp, synthetic construct
  source 1..224: mol_type = other DNA
gtcatcttagtcattacctggtttaagagctaagctggaaacagcatagcaagtttaaataaggctagtccgttatcaactcgagagagtggcaccgagtcggtgcttgagttacaacgaacacctcagcttatcgtcgtcatccttgtaatcgtaatgactaagatgtcatctctttgacgcggttctatctagttacgcgttaaaccaactagaaatttttt

SEQ ID NO: 336 - DNA, 224 bp, synthetic construct
  source 1..224: mol_type = other DNA
gtcatcttagtcattacctggtttaagagctaagctggaaacagcatagcaagtttaaataaggctagtccgttatcaactggaaacagtggcaccgagtcggtgcttgagttacaacgaacacctcagcttatcgtcgtcatccttgtaatcgtaatgactaagatgtcatctctttgacgcggttctatctagttacgcgttaaaccaactagaaatttttt

SEQ ID NO: 337 - DNA, 224 bp, synthetic construct
  source 1..224: mol_type = other DNA
gtcatcttagtcattacctggtttaagagctaagctggaaacagcatagcaagtttaaataaggctagtccgttatcagctggaaacagcggcaccgagtcggtgcttgagttacaacgaacacctcagcttatcgtcgtcatccttgtaatcgtaatgactaagatgtcatctctttgacgcggttctatctagttacgcgttaaaccaactagaaatttttt

SEQ ID NO: 338 - DNA, 224 bp, synthetic construct
  source 1..224: mol_type = other DNA
gtcatcttagtcattacctggtttaagagctaagctggaaacagcatagcaagtttaaataaggctagtccgttatcaactcgaaagagtggcaccgagtcggtgcttgagttacaacgaacacctcagcttatcgtcgtcatccttgtaatcgtaatgactaagatgtcatctctttgacgcggttctatctagttacgcgttaaaccaactagaaatttttt

SEQ ID NO: 339 - DNA, 224 bp, synthetic construct
  source 1..224: mol_type = other DNA
gtcatcttagtcattacctggtttaagagctaagctggaaacagcatagcaagtttaaataaggctagtccgttatcaacgtgaaaacgtgactccgagtcggagtttgagttacaacgaacacctcagcttatcgtcgtcatccttgtaatcgtaatgactaagatgtcatctctttgacgcggttctatctagttacgcgttaaaccaactagaaatttttt

SEQ ID NO: 340 - DNA, 224 bp, synthetic construct
  source 1..224: mol_type = other DNA
gtcatcttagtcattacctggtttaagagctaagctggaaacagcatagcaagtttaaataaggctagtccgttatcaactcgaaagagtgcaaccgagtcggttgttgagttacaacgaacacctcagcttatcgtcgtcatccttgtaatcgtaatgactaagatgtcatctctttgacgcggttctatctagttacgcgttaaaccaactagaaatttttt

SEQ ID NO: 341 - DNA, 224 bp, synthetic construct
  source 1..224: mol_type = other DNA
gtcatcttagtcattacctggtttaagagctaagctggaaacagcatagcaagtttaaataaggctagtccgttatcaacttgaaaaagtggcaccgagtcggtgcttgagttacaacgaacacctcagcttatcgtcgtcatccttgtaatcgtaatgactaagatgtcatctctttgacgcggttctatctagttacgcgttaaaccaactagaaatttttt

SEQ ID NO: 342 - DNA, 198 bp, synthetic construct
  source 1..198: mol_type = other DNA
gatgtctgcaggccagatgagtttaagagctaagctggaaacagcatagcaagtttaaataaggctagtccgttatcaactcgagagagtggcaccgagtcggtgctaatgtgccatctggagcactcatctggcctgcagaacaatctcttgacgcggttctatctagttacgcgttaaaccaactagaaatttttt

SEQ ID NO: 343 - DNA, 198 bp, synthetic construct
  source 1..198: mol_type = other DNA
gatgtctgcaggccagatgagtttaagagctaagctggaaacagcatagcaagtttaaataaggctagtccgttatcaactggaaacagtggcaccgagtcggtgctaatgtgccatctggagcactcatctggcctgcagaacaatctcttgacgcggttctatctagttacgcgttaaaccaactagaaatttttt

SEQ ID NO: 344 - DNA, 198 bp, synthetic construct
  source 1..198: mol_type = other DNA
gatgtctgcaggccagatgagtttaagagctaagctggaaacagcatagcaagtttaaataaggctagtccgttatcagctggaaacagcggcaccgagtcggtgctaatgtgccatctggagcactcatctggcctgcagaacaatctcttgacgcggttctatctagttacgcgttaaaccaactagaaatttttt

SEQ ID NO: 345 - DNA, 198 bp, synthetic construct
  source 1..198: mol_type = other DNA
gatgtctgcaggccagatgagtttaagagctaagctggaaacagcatagcaagtttaaataaggctagtccgttatcaactcgaaagagtggcaccgagtcggtgctaatgtgccatctggagcactcatctggcctgcagaacaatctcttgacgcggttctatctagttacgcgttaaaccaactagaaatttttt

SEQ ID NO: 346 - DNA, 198 bp, synthetic construct
  source 1..198: mol_type = other DNA
gatgtctgcaggccagatgagtttaagagctaagctggaaacagcatagcaagtttaaataaggctagtccgttatcaacgtgaaaacgtgactccgagtcggagttaatgtgccatctggagcactcatctggcctgcagaacaatctcttgacgcggttctatctagttacgcgttaaaccaactagaaatttttt

SEQ ID NO: 347 - DNA, 198 bp, synthetic construct
  source 1..198: mol_type = other DNA
gatgtctgcaggccagatgagtttaagagctaagctggaaacagcatagcaagtttaaataaggctagtccgttatcaactcgaaagagtgcaaccgagtcggttgtaatgtgccatctggagcactcatctggcctgcagaacaatctcttgacgcggttctatctagttacgcgttaaaccaactagaaatttttt

SEQ ID NO: 348 - DNA, 198 bp, synthetic construct
  source 1..198: mol_type = other DNA
gatgtctgcaggccagatgagtttaagagctaagctggaaacagcatagcaagtttaaataaggctagtccgttatcaacttgaaaaagtggcaccgagtcggtgctaatgtgccatctggagcactcatctggcctgcagaacaatctcttgacgcggttctatctagttacgcgttaaaccaactagaaatttttt

SEQ ID NO: 349 - DNA, 211 bp, synthetic construct
  source 1..211: mol_type = other DNA
gatgtctgcaggccagatgagttttagagctagaaatagcaagttaaaataaggctagtccgttatcaacttgaaaaagtggcaccgagtcggtgcaatgtgccatctggagcactcacttatcgtcgtcatccttgtaatctctggcctgcagaacaatctcttgacgcggttctatctagttacgcgttaaaccaactagaaatttttt

SEQ ID NO: 350 - DNA, 222 bp, synthetic construct
  source 1..222: mol_type = other DNA
gatgtctgcaggccagatgagtttaagagctaagctggaaacagcatagcaagtttaaataaggctagtccgttatcaactcgagagagtggcaccgagtcggtgctaatgtgccatctggagcactcacttatcgtcgtcatccttgtaatctctggcctgcagaacaatctcttgacgcggttctatctagttacgcgttaaaccaactagaaatttttt

SEQ ID NO: 351 - DNA, 222 bp, synthetic construct
  source 1..222: mol_type = other DNA
gatgtctgcaggccagatgagtttaagagctaagctggaaacagcatagcaagtttaaataaggctagtccgttatcaactggaaacagtggcaccgagtcggtgctaatgtgccatctggagcactcacttatcgtcgtcatccttgtaatctctggcctgcagaacaatctcttgacgcggttctatctagttacgcgttaaaccaactagaaatttttt

SEQ ID NO: 352 - DNA, 222 bp, synthetic construct
  source 1..222: mol_type = other DNA
gatgtctgcaggccagatgagtttaagagctaagctggaaacagcatagcaagtttaaataaggctagtccgttatcagctggaaacagcggcaccgagtcggtgctaatgtgccatctggagcactcacttatcgtcgtcatccttgtaatctctggcctgcagaacaatctcttgacgcggttctatctagttacgcgttaaaccaactagaaatttttt

SEQ ID NO: 353 - DNA, 222 bp, synthetic construct
  source 1..222: mol_type = other DNA
gatgtctgcaggccagatgagtttaagagctaagctggaaacagcatagcaagtttaaataaggctagtccgttatcaactcgaaagagtggcaccgagtcggtgctaatgtgccatctggagcactcacttatcgtcgtcatccttgtaatctctggcctgcagaacaatctcttgacgcggttctatctagttacgcgttaaaccaactagaaatttttt

SEQ ID NO: 354 - DNA, 222 bp, synthetic construct
  source 1..222: mol_type = other DNA
gatgtctgcaggccagatgagtttaagagctaagctggaaacagcatagcaagtttaaataaggctagtccgttatcaacgtgaaaacgtgactccgagtcggagttaatgtgccatctggagcactcacttatcgtcgtcatccttgtaatctctggcctgcagaacaatctcttgacgcggttctatctagttacgcgttaaaccaactagaaatttttt

SEQ ID NO: 355 - DNA, 222 bp, synthetic construct
  source 1..222: mol_type = other DNA
gatgtctgcaggccagatgagtttaagagctaagctggaaacagcatagcaagtttaaataaggctagtccgttatcaactcgaaagagtgcaaccgagtcggttgtaatgtgccatctggagcactcacttatcgtcgtcatccttgtaatctctggcctgcagaacaatctcttgacgcggttctatctagttacgcgttaaaccaactagaaatttttt

SEQ ID NO: 356 - DNA, 222 bp, synthetic construct
  source 1..222: mol_type = other DNA
gatgtctgcaggccagatgagtttaagagctaagctggaaacagcatagcaagtttaaataaggctagtccgttatcaacttgaaaaagtggcaccgagtcggtgctaatgtgccatctggagcactcacttatcgtcgtcatccttgtaatctctggcctgcagaacaatctcttgacgcggttctatctagttacgcgttaaaccaactagaaatttttt

SEQ ID NO: 357 - DNA, 187 bp, synthetic construct
  source 1..187: mol_type = other DNA
gatgtctgcaggccagatgagttttagagctagaaatagcaagttaaaataaggctagtccgttatcaacttgaaaaagtggcaccgagtcggtgcgtgtgtccctctgacaatgtgctctggcctgcagaacaatctcttgacgcggttctatctagttacgcgttaaaccaactagaaatttttt

SEQ ID NO: 358 - DNA, 198 bp, synthetic construct
  source 1..198: mol_type = other DNA
gatgtctgcaggccagatgagtttaagagctaagctggaaacagcatagcaagtttaaataaggctagtccgttatcaactcgagagagtggcaccgagtcggtgctgtgtgtccctctgacaatgtgctctggcctgcagaacaatctcttgacgcggttctatctagttacgcgttaaaccaactagaaatttttt

SEQ ID NO: 359 - DNA, 198 bp, synthetic construct
  source 1..198: mol_type = other DNA
gatgtctgcaggccagatgagtttaagagctaagctggaaacagcatagcaagtttaaataaggctagtccgttatcaactggaaacagtggcaccgagtcggtgctgtgtgtccctctgacaatgtgctctggcctgcagaacaatctcttgacgcggttctatctagttacgcgttaaaccaactagaaatttttt

SEQ ID NO: 360 - DNA, 198 bp, synthetic construct
  source 1..198: mol_type = other DNA
gatgtctgcaggccagatgagtttaagagctaagctggaaacagcatagcaagtttaaataaggctagtccgttatcagctggaaacagcggcaccgagtcggtgctgtgtgtccctctgacaatgtgctctggcctgcagaacaatctcttgacgcggttctatctagttacgcgttaaaccaactagaaatttttt

SEQ ID NO: 361 - DNA, 198 bp, synthetic construct
  source 1..198: mol_type = other DNA
gatgtctgcaggccagatgagtttaagagctaagctggaaacagcatagcaagtttaaataaggctagtccgttatcaactcgaaagagtggcaccgagtcggtgctgtgtgtccctctgacaatgtgctctggcctgcagaacaatctcttgacgcggttctatctagttacgcgttaaaccaactagaaatttttt

SEQ ID NO: 362 - DNA, 198 bp, synthetic construct
  source 1..198: mol_type = other DNA
gatgtctgcaggccagatgagtttaagagctaagctggaaacagcatagcaagtttaaataaggctagtccgttatcaacgtgaaaacgtgactccgagtcggagttgtgtgtccctctgacaatgtgctctggcctgcagaacaatctcttgacgcggttctatctagttacgcgttaaaccaactagaaatttttt

SEQ ID NO: 363 - DNA, 198 bp, synthetic construct
  source 1..198: mol_type = other DNA
gatgtctgcaggccagatgagtttaagagctaagctggaaacagcatagcaagtttaaataaggctagtccgttatcaactcgaaagagtgcaaccgagtcggttgtgtgtgtccctctgacaatgtgctctggcctgcagaacaatctcttgacgcggttctatctagttacgcgttaaaccaactagaaatttttt

SEQ ID NO: 364 - DNA, 198 bp, synthetic construct
  source 1..198: mol_type = other DNA
gatgtctgcaggccagatgagtttaagagctaagctggaaacagcatagcaagtttaaataaggctagtccgttatcaacttgaaaaagtggcaccgagtcggtgctgtgtgtccctctgacaatgtgctctggcctgcagaacaatctcttgacgcggttctatctagttacgcgttaaaccaactagaaatttttt

SEQ ID NO: 365 - DNA, 206 bp, synthetic construct
  source 1..206: mol_type = other DNA
gcattttcaggaggaagcgagttttagagctagaaatagcaagttaaaataaggctagtccgttatcaacttgaaaaagtggcaccgagtcggtgctgtctgaagccatcccttatcgtcgtcatccttgtaatccttcctcctgaaaataactctctttgacgcggttctatctagttacgcgttaaaccaactagaaatttttt

SEQ ID NO: 366 - DNA, 217 bp, synthetic construct
  source 1..217: mol_type = other DNA
gcattttcaggaggaagcgagtttaagagctaagctggaaacagcatagcaagtttaaataaggctagtccgttatcaactcgagagagtggcaccgagtcggtgcttgtctgaagccatcccttatcgtcgtcatccttgtaatccttcctcctgaaaataactctctttgacgcggttctatctagttacgcgttaaaccaactagaaatttttt

SEQ ID NO: 367 - DNA, 217 bp, synthetic construct
  source 1..217: mol_type = other DNA
gcattttcaggaggaagcgagtttaagagctaagctggaaacagcatagcaagtttaaataaggctagtccgttatcaactggaaacagtggcaccgagtcggtgcttgtctgaagccatcccttatcgtcgtcatccttgtaatccttcctcctgaaaataactctctttgacgcggttctatctagttacgcgttaaaccaactagaaatttttt

SEQ ID NO: 368 - DNA, 217 bp, synthetic construct
  source 1..217: mol_type = other DNA
gcattttcaggaggaagcgagtttaagagctaagctggaaacagcatagcaagtttaaataaggctagtccgttatcagctggaaacagcggcaccgagtcggtgcttgtctgaagccatcccttatcgtcgtcatccttgtaatccttcctcctgaaaataactctctttgacgcggttctatctagttacgcgttaaaccaactagaaatttttt

SEQ ID NO: 369 - DNA, 217 bp, synthetic construct
  source 1..217: mol_type = other DNA
gcattttcaggaggaagcgagtttaagagctaagctggaaacagcatagcaagtttaaataaggctagtccgttatcaactcgaaagagtggcaccgagtcggtgcttgtctgaagccatcccttatcgtcgtcatccttgtaatccttcctcctgaaaataactctctttgacgcggttctatctagttacgcgttaaaccaactagaaatttttt

SEQ ID NO: 370 - DNA, 217 bp, synthetic construct
  source 1..217: mol_type = other DNA
gcattttcaggaggaagcgagtttaagagctaagctggaaacagcatagcaagtttaaataaggctagtccgttatcaacgtgaaaacgtgactccgagtcggagtttgtctgaagccatcccttatcgtcgtcatccttgtaatccttcctcctgaaaataactctctttgacgcggttctatctagttacgcgttaaaccaactagaaatttttt

SEQ ID NO: 371 - DNA, 217 bp, synthetic construct
  source 1..217: mol_type = other DNA
gcattttcaggaggaagcgagtttaagagctaagctggaaacagcatagcaagtttaaataaggctagtccgttatcaactcgaaagagtgcaaccgagtcggttgttgtctgaagccatcccttatcgtcgtcatccttgtaatccttcctcctgaaaataactctctttgacgcggttctatctagttacgcgttaaaccaactagaaatttttt

SEQ ID NO: 372 - DNA, 217 bp, synthetic construct
  source 1..217: mol_type = other DNA
gcattttcaggaggaagcgagtttaagagctaagctggaaacagcatagcaagtttaaataaggctagtccgttatcaacttgaaaaagtggcaccgagtcggtgcttgtctgaagccatcccttatcgtcgtcatccttgtaatccttcctcctgaaaataactctctttgacgcggttctatctagttacgcgttaaaccaactagaaatttttt

SEQ ID NO: 373 - DNA, 212 bp, synthetic construct
  source 1..212: mol_type = other DNA
gattcctggtgccagaaacagttttagagctagaaatagcaagttaaaataaggctagtccgttatcaacttgaaaaagtggcaccgagtcggtgctctgccctcccgtcacccctgtcttatcgtcgtcatccttgtaatcttctggcaccaggacctcttctttgacgcggttctatctagttacgcgttaaaccaactagaaatttttt

SEQ ID NO: 374 - DNA, 223 bp, synthetic construct
  source 1..223: mol_type = other DNA
gattcctggtgccagaaacagtttaagagctaagctggaaacagcatagcaagtttaaataaggctagtccgttatcaactcgagagagtggcaccgagtcggtgcttctgccctcccgtcacccctgtcttatcgtcgtcatccttgtaatcttctggcaccaggacctcttctttgacgcggttctatctagttacgcgttaaaccaactagaaatttttt

SEQ ID NO: 375 - DNA, 223 bp, synthetic construct
  source 1..223: mol_type = other DNA
gattcctggtgccagaaacagtttaagagctaagctggaaacagcatagcaagtttaaataaggctagtccgttatcaactggaaacagtggcaccgagtcggtgcttctgccctcccgtcacccctgtcttatcgtcgtcatccttgtaatcttctggcaccaggacctcttctttgacgcggttctatctagttacgcgttaaaccaactagaaatttttt

SEQ ID NO: 376 - DNA, 223 bp, synthetic construct
  source 1..223: mol_type = other DNA
gattcctggtgccagaaacagtttaagagctaagctggaaacagcatagcaagtttaaataaggctagtccgttatcagctggaaacagcggcaccgagtcggtgcttctgccctcccgtcacccctgtcttatcgtcgtcatccttgtaatcttctggcaccaggacctcttctttgacgcggttctatctagttacgcgttaaaccaactagaaatttttt

SEQ ID NO: 377 - DNA, 223 bp, synthetic construct
  source 1..223: mol_type = other DNA
gattcctggtgccagaaacagtttaagagctaagctggaaacagcatagcaagtttaaataaggctagtccgttatcaactcgaaagagtggcaccgagtcggtgcttctgccctcccgtcacccctgtcttatcgtcgtcatccttgtaatcttctggcaccaggacctcttctttgacgcggttctatctagttacgcgttaaaccaactagaaatttttt

SEQ ID NO: 378 - DNA, 223 bp, synthetic construct
  source 1..223: mol_type = other DNA
gattcctggtgccagaaacagtttaagagctaagctggaaacagcatagcaagtttaaataaggctagtccgttatcaacgtgaaaacgtgactccgagtcggagtttctgccctcccgtcacccctgtcttatcgtcgtcatccttgtaatcttctggcaccaggacctcttctttgacgcggttctatctagttacgcgttaaaccaactagaaatttttt

SEQ ID NO: 379 - DNA, 223 bp, synthetic construct
  source 1..223: mol_type = other DNA
gattcctggtgccagaaacagtttaagagctaagctggaaacagcatagcaagtttaaataaggctagtccgttatcaactcgaaagagtgcaaccgagtcggttgttctgccctcccgtcacccctgtcttatcgtcgtcatccttgtaatcttctggcaccaggacctcttctttgacgcggttctatctagttacgcgttaaaccaactagaaatttttt

SEQ ID NO: 380 - DNA, 223 bp, synthetic construct
  source 1..223: mol_type = other DNA
gattcctggtgccagaaacagtttaagagctaagctggaaacagcatagcaagtttaaataaggctagtccgttatcaacttgaaaaagtggcaccgagtcggtgcttctgccctcccgtcacccctgtcttatcgtcgtcatccttgtaatcttctggcaccaggacctcttctttgacgcggttctatctagttacgcgttaaaccaactagaaatttttt

SEQ ID NO: 381 - DNA, 20 bp, synthetic construct
  source 1..20: mol_type = other DNA
gaccctggaaaagctgatga

SEQ ID NO: 382 - DNA, 20 bp, synthetic construct
  source 1..20: mol_type = other DNA
gctgctgctggaaggacttg

SEQ ID NO: 383 - DNA, 21 bp, synthetic construct
  source 1..21: mol_type = other DNA
gctgctgctgctgctgctgga

SEQ ID NO: 384 - DNA, 20 bp, synthetic construct
  source 1..20: mol_type = other DNA
gctgctgctgctgctgctgc

SEQ ID NO: 385 - DNA, 20 bp, synthetic construct
  source 1..20: mol_type = other DNA
ggcggcggcggcggcggtgg

SEQ ID NO: 386 - DNA, 20 bp, synthetic construct
  source 1..20: mol_type = other DNA
tgaggaagctgaggaggcgg

SEQ ID NO: 387 - DNA, 20 bp, synthetic construct
  source 1..20: mol_type = other DNA
ggcggctgaggaagctgagg

SEQ ID NO: 388 - DNA, 21 bp, synthetic construct
  source 1..21: mol_type = other DNA
gcaagactaacctggccaaca

SEQ ID NO: 389 - DNA, 21 bp, synthetic construct
  source 1..21: mol_type = other DNA
gtccggagttcaagactaacc

SEQ ID NO: 390 - DNA, 21 bp, synthetic construct
  source 1..21: mol_type = other DNA
gaaggtggatcacctgaggtc

SEQ ID NO: 391 - DNA, 21 bp, synthetic construct
  source 1..21: mol_type = other DNA
gtctggagtagctgggattac

SEQ ID NO: 392 - DNA, 21 bp, synthetic construct
  source 1..21: mol_type = other DNA
gcaggcgcgcgacaccacgcc

SEQ ID NO: 393 - DNA, 20 bp, synthetic construct
  source 1..20: mol_type = other DNA
gaaggacttgagggactcga

SEQ ID NO: 394 - DNA, 21 bp, synthetic construct
  source 1..21: mol_type = other DNA
gtgaggaagctgaggaggcgg

SEQ ID NO: 395 - DNA, 20 bp, synthetic construct
  source 1..20: mol_type = other DNA
gctgttgctgctgctgctgc

SEQ ID NO: 396 - DNA, 19 bp, synthetic construct
  source 1..19: mol_type = other DNA
ggccttcatcagcttttcc

SEQ ID NO: 397 - DNA, 19 bp, synthetic construct
  source 1..19: mol_type = other DNA
ggctttcatcagcttttcc

SEQ ID NO: 398 - DNA, 19 bp, synthetic construct
  source 1..19: mol_type = other DNA
gggactcgaaggccttcat

SEQ ID NO: 399 - DNA, 20 bp, synthetic construct
  source 1..20: mol_type = other DNA
ggaaggacttgagggactcg

SEQ ID NO: 400 - DNA, 19 bp, synthetic construct
  source 1..19: mol_type = other DNA
gctgctgctggaaggactt

SEQ ID NO: 401 - DNA, 20 bp, synthetic construct
  source 1..20: mol_type = other DNA
gctgctgctgctgctgctgg

SEQ ID NO: 402 - DNA, 21 bp, synthetic construct
  source 1..21: mol_type = other DNA
gctggaaggacttgagggact

SEQ ID NO: 403 - DNA, 21 bp, synthetic construct
  source 1..21: mol_type = other DNA
gctgctgctgctggaaggact

SEQ ID NO: 404 - DNA, 21 bp, synthetic construct
  source 1..21: mol_type = other DNA
ctgctgctgctgctgctggaa

SEQ ID NO: 405 - DNA, 21 bp, synthetic construct
  source 1..21: mol_type = other DNA
gctgctgctgctgctgctgct

SEQ ID NO: 406 - DNA, 19 bp, synthetic construct
  source 1..19: mol_type = other DNA
gctgctggaaggacttgag

SEQ ID NO: 407 - DNA, 19 bp, synthetic construct
  source 1..19: mol_type = other DNA
gctgctggaaggacttcag

SEQ ID NO: 408 - DNA, 19 bp, synthetic construct
  source 1..19: mol_type = other DNA
gctgctgaaaggacttcag

SEQ ID NO: 409 - DNA, 19 bp, synthetic construct
  source 1..19: mol_type = other DNA
gctgttggaaggacttcag

SEQ ID NO: 410 - DNA, 21 bp, synthetic construct
  source 1..21: mol_type = other DNA
gtcccaaagtgctgagattat

SEQ ID NO: 411 - DNA, 21 bp, synthetic construct
  source 1..21: mol_type = other DNA
gtgtattttttagtagatact

SEQ ID NO: 412 - DNA, 20 bp, synthetic construct
  source 1..20: mol_type = other DNA
gattctcctgccgcagcctc

SEQ ID NO: 413 - DNA, 21 bp, synthetic construct
  source 1..21: mol_type = other DNA
gcgacaccacgcccggctaac

SEQ ID NO: 414 - DNA, 250 bp, synthetic construct
  source 1..250: mol_type = other DNA
gagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccg

SEQ ID NO: 415 - DNA, 301 bp, synthetic construct
  source 1..301: mol_type = other DNA
gcctgaggcgtggggccgcctcccaaagacttctgggagggcggtgcggctcaggctctgccccgcctccggggctatttgcatacgaccatttccagtaattcccagcagccaccgtagctatatttggtagaacaacgagcactttctcaactccagtcaataactacgttagttgcattacacattgggctaatataaatagaggttaaatctctaggtcatttaagagaagtcggcctatgtgtacagacatttgttccaggggctttaaatagctggtggtggaactcaatattcg

SEQ ID NO: 416 - DNA, 301 bp, synthetic construct
  source 1..301: mol_type = other DNA
aagtccgcggcacgagaaatcaaagccccggggcctgggtcccacgcggggtcccttacccagggtgccccgggcgctcatttgcatgtcccacccaacaggtaaacctgacagatcggtcgcggccaggtacggcctggcggtcagagcaccaaacttacgagccttgtgatgagttccgttacatgaaattctcctaaaggctccaagatggacaggaaagcgctcgattaggttaccgtaaggaaaacaaatgagaaactcccgtgccttataagacctggggacggacttatttgcg

SEQ ID NO: 417 - DNA, 301 bp, synthetic construct
  source 1..301: mol_type = other DNA
aaattgagtcatctgacagaaattatctttggcaaggttttagtcctagggttaccagatggaatacaggacatccatttaaatttgaatttcagataaacagttaacacttctcaaggataaatatgcctcaaatattgcacgggacatatttatactaaaaaaaaagtgtttttttttttcctgcgattcaaacttaactggtgtcctgcatttgtatttgttaaatctgtcaatcctatctcagtttcctttgatggaatgtacctctgtgctaatatttaaaaataggttacatttg

SEQ ID NO: 418 - DNA, 56 bp, synthetic construct
  source 1..56: mol_type = other DNA
gggtcaggagccccccccctgaacccaggataaccctcaaagtcggggggcaaccc

SEQ ID NO: 419 - DNA, 51 bp, synthetic construct
  source 1..51: mol_type = other DNA
gggtcaggagccccccccctgaacccaggataaccctcaaagtcggggggc

SEQ ID NO: 420 - DNA, 60 bp, synthetic construct
  source 1..60: mol_type = other DNA
gtcagggtcaggagccccccccctgaacccaggataaccctcaaagtcggggggcaaccc

SEQ ID NO: 421 - DNA, 56 bp, synthetic construct
  source 1..56: mol_type = other DNA
gggtcaggagccccccccctgaacccaggaaaaccctcaaagtcggggggcaaccc

SEQ ID NO: 422 - DNA, 56 bp, synthetic construct
  source 1..56: mol_type = other DNA
gggtcaggagccccccccctgcacccaggaaaaccctcaaagtcggggggcaaccc

SEQ ID NO: 423 - DNA, 56 bp, synthetic construct
  source 1..56: mol_type = other DNA
gggtcaggagccccccccctgcacccaggataaccctcaaagtcggggggcaaccc

SEQ ID NO: 424 - DNA, 60 bp, synthetic construct
  source 1..60: mol_type = other DNA
gtcagggtcaggagccccccccctgaacccaggaaaaccctcaaagtcggggggcaaccc

SEQ ID NO: 425 - DNA, 60 bp, synthetic construct
  source 1..60: mol_type = other DNA
gtcagggtcaggagccccccccctgcacccaggaaaaccctcaaagtcggggggcaaccc

SEQ ID NO: 426 - DNA, 60 bp, synthetic construct
  source 1..60: mol_type = other DNA
gtcagggtcaggagccccccccctgcacccaggataaccctcaaagtcggggggcaaccc

SEQ ID NO: 427 - DNA, 19 bp, synthetic construct
  source 1..19: mol_type = other DNA
gggctgggatgggaaaggg

SEQ ID NO: 428 - DNA, 20 bp, synthetic construct
  source 1..20: mol_type = other DNA
gggacagggcagggacaggg

SEQ ID NO: 429 - DNA, 21 bp, synthetic construct
  source 1..21: mol_type = other DNA
gggtccgggtctgggtctggg

SEQ ID NO: 430 - DNA, 19 bp, synthetic construct
  source 1..19: mol_type = other DNA
gggctctgggtgggccggg

SEQ ID NO: 431 - DNA, 17 bp, synthetic construct
  source 1..17: mol_type = other DNA
gggtgggctgggaaggg

SEQ ID NO: 432 - DNA, 17 bp, synthetic construct
  source 1..17: mol_type = other DNA
gggagggagggctaggg

SEQ ID NO: 433 - DNA, 18 bp, synthetic construct
  source 1..18: mol_type = other DNA
gggctgggctgggcaggg

SEQ ID NO: 434 - DNA, 17 bp, synthetic construct
  source 1..17: mol_type = other DNA
gggcagggctgggaggg

SEQ ID NO: 435 - DNA, 16 bp, synthetic construct
  source 1..16: mol_type = other DNA
gggtgggagggctggg

SEQ ID NO: 436 - DNA, 19 bp, synthetic construct
  source 1..19: mol_type = other DNA
gggcagggtctgggctggg

SEQ ID NO: 437 - DNA, 12 bp, synthetic construct
  source 1..12: mol_type = other DNA
tggtggtggtgg

SEQ ID NO: 438 - DNA, 42 bp, synthetic construct
  source 1..42: mol_type = other DNA
ttgacgcggttctatctagttacgcgttaaaccaactagaaa

SEQ ID NO: 439 - DNA, 44 bp, synthetic construct
  source 1..44: mol_type = other DNA
cgcgagtctaggggataacgcgttaaacttcctagaaggcggtt

SEQ ID NO: 440 - DNA, 44 bp, synthetic construct
  source 1..44: mol_type = other DNA
cgcggatctagattgtaacgcgttaaaccatctagaaggcggtt

SEQ ID NO: 441 - DNA, 44 bp, synthetic construct
  source 1..44: mol_type = other DNA
cgcgtcgctaccgcccggcgcgttaaacacactagaaggcggtt

SEQ ID NO: 442 - DNA, 37 bp, synthetic construct
  source 1..37: mol_type = other DNA
cgcggttctatctagttacgcgttaaaccaactagaa

SEQ ID NO: 443 - DNA, 42 bp, synthetic construct
  source 1..42: mol_type = other DNA
ttgacgcgcttctatctagttacgcgttaaaccaactagaaa

SEQ ID NO: 444 - DNA, 42 bp, synthetic construct
  source 1..42: mol_type = other DNA
ttgacgcggttctatctacttacgcgttaaaccaactagaaa

SEQ ID NO: 445 - DNA, 81 bp, synthetic construct
  source 1..81: mol_type = other DNA
ggcggggctcgttggtctaggggtatgattctcgcttcgggtgcgagaggtcccgggttcaaatcccggacgagccccgcc

SEQ ID NO: 446 - DNA, 43 bp, synthetic construct
  source 1..43: mol_type = other DNA
gcgtaacctccatccgagttgcaagagagggaaacgcagtctc

SEQ ID NO: 447 - DNA, 160 bp, synthetic construct
  source 1..160: mol_type = other DNA
ggaattgcgggaaaggggtcaacagccgttcagtaccaagtctcaggggaaactttgagatggccttgcaaagggtatggtaataagctgacggacatggtcctaaccacgcagccaagtcctaagtcaacagatcttctgttgatatggatgcagttca

SEQ ID NO: 448 - DNA, 18 bp, synthetic construct
  source 1..18: mol_type = other DNA
tctctctcacacacacac

SEQ ID NO: 449 - DNA, 18 bp, synthetic construct
  source 1..18: mol_type = other DNA
tcatctctacacacacac

SEQ ID NO: 450 - DNA, 18 bp, synthetic construct
  source 1..18: mol_type = other DNA
caatcactacacacacac

SEQ ID NO: 451 - DNA, 18 bp, synthetic construct
  source 1..18: mol_type = other DNA
aactctctacacacacac

SEQ ID NO: 452 - DNA, 18 bp, synthetic construct
  source 1..18: mol_type = other DNA
aacaatctacacacacac

SEQ ID NO: 453 - DNA, 18 bp, synthetic construct
  source 1..18: mol_type = other DNA
cctcttctacacacacac

SEQ ID NO: 454 - DNA, 161 bp, synthetic construct
  source 1..161: mol_type = other DNA
gaccctggaaaagctgatgagttttagagctagaaatagcaagttaaaataaggctagtccgttatcaacttgaaaaagtggcaccgagtcggtgcgcggtggcggctgttgctgctgctgctggaaggacttgagggactcgaaggctttcatcagcttt

SEQ ID NO: 455 - DNA, 163 bp, synthetic construct
  source 1..163: mol_type = other DNA
gaccctggaaaagctgatgagttttagagctagaaatagcaagttaaaataaggctagtccgttatcaacttgaaaaagtggcaccgagtcggtgcgcggtggcggctgttgctgctgctgctggaaggacttgagggactcgaaggctttcatcagcttttc

SEQ ID NO: 456 - DNA, 165 bp, synthetic construct
  source 1..165: mol_type = other DNA
gaccctggaaaagctgatgagttttagagctagaaatagcaagttaaaataaggctagtccgttatcaacttgaaaaagtggcaccgagtcggtgcgcggtggcggctgttgctgctgctgctggaaggacttgagggactcgaaggctttcatcagcttttcca

SEQ ID NO: 457 - DNA, 166 bp, synthetic construct
  source 1..166: mol_type = other DNA
gaccctggaaaagctgatgagttttagagctagaaatagcaagttaaaataaggctagtccgttatcaacttgaaaaagtggcaccgagtcggtgcgcggtggcggctgttgctgctgctgctggaaggacttgagggactcgaaggctttcatcagcttttccag

SEQ ID NO: 458 - DNA, 167 bp, synthetic construct
  source 1..167: mol_type = other DNA
gaccctggaaaagctgatgagttttagagctagaaatagcaagttaaaataaggctagtccgttatcaacttgaaaaagtggcaccgagtcggtgcgcggtggcggctgttgctgctgctgctggaaggacttgagggactcgaaggctttcatcagcttttccagg

SEQ ID NO: 459 - DNA, 165 bp, synthetic construct
  source 1..165: mol_type = other DNA
gaccctggaaaagctgatgagttttagagctagaaatagcaagttaaaataaggctagtccgttatcaacttgaaaaagtggcaccgagtcggtgcggcggcggtggcggctgttgctgctgctgctggaaggacttgagggactcgaaggctttcatcagcttt

SEQ ID NO: 460 - DNA, 166 bp, synthetic construct
  source 1..166: mol_type = other DNA
gaccctggaaaagctgatgagttttagagctagaaatagcaagttaaaataaggctagtccgttatcaacttgaaaaagtggcaccgagtcggtgcggcggcggtggcggctgttgctgctgctgctggaaggacttgagggactcgaaggctttcatcagctttt

SEQ ID NO: 461 - DNA, 168 bp, synthetic construct
  source 1..168: mol_type = other DNA
gaccctggaaaagctgatgagttttagagctagaaatagcaagttaaaataaggctagtccgttatcaacttgaaaaagtggcaccgagtcggtgcggcggcggtggcggctgttgctgctgctgctggaaggacttgagggactcgaaggctttcatcagcttttcc

SEQ ID NO: 462 - DNA, 169 bp, synthetic construct
  source 1..169: mol_type = other DNA
gaccctggaaaagctgatgagttttagagctagaaatagcaagttaaaataaggctagtccgttatcaacttgaaaaagtggcaccgagtcggtgcggcggcggtggcggctgttgctgctgctgctggaaggacttgagggactcgaaggctttcatcagcttttcca

SEQ ID NO: 463 - DNA, 170 bp, synthetic construct
  source 1..170: mol_type = other DNA
gaccctggaaaagctgatgagttttagagctagaaatagcaagttaaaataaggctagtccgttatcaacttgaaaaagtggcaccgagtcggtgcggcggcggtggcggctgttgctgctgctgctggaaggacttgagggactcgaaggctttcatcagcttttccag

SEQ ID NO: 464 - DNA, 171 bp, synthetic construct
  source 1..171: mol_type = other DNA
gaccctggaaaagctgatgagttttagagctagaaatagcaagttaaaataaggctagtccgttatcaacttgaaaaagtggcaccgagtcggtgcggcggcggtggcggctgttgctgctgctgctggaaggacttgagggactcgaaggctttcatcagcttttccagg

SEQ ID NO: 465 - DNA, 170 bp, synthetic construct
  source 1..170: mol_type = other DNA
gaccctggaaaagctgatgagttttagagctagaaatagcaagttaaaataaggctagtccgttatcaacttgaaaaagtggcaccgagtcggtgcgcggcggcggcggtggcggctgttgctgctgctgctggaaggacttgagggactcgaaggctttcatcagcttt

SEQ ID NO: 466 - DNA, 171 bp, synthetic construct
  source 1..171: mol_type = other DNA
gaccctggaaaagctgatgagttttagagctagaaatagcaagttaaaataaggctagtccgttatcaacttgaaaaagtggcaccgagtcggtgcgcggcggcggcggtggcggctgttgctgctgctgctggaaggacttgagggactcgaaggctttcatcagctttt

SEQ ID NO: 467 - DNA, 172 bp, synthetic construct
  source 1..172: mol_type = other DNA
gaccctggaaaagctgatgagttttagagctagaaatagcaagttaaaataaggctagtccgttatcaacttgaaaaagtggcaccgagtcggtgcgcggcggcggcggtggcggctgttgctgctgctgctggaaggacttgagggactcgaaggctttcatcagcttttc

SEQ ID NO: 468 - DNA, 173 bp, synthetic construct
  source 1..173: mol_type = other DNA
gaccctggaaaagctgatgagttttagagctagaaatagcaagttaaaataaggctagtccgttatcaacttgaaaaagtggcaccgagtcggtgcgcggcggcggcggtggcggctgttgctgctgctgctggaaggacttgagggactcgaaggctttcatcagcttttcc

SEQ ID NO: 469 - DNA, 174 bp, synthetic construct
  source 1..174: mol_type = other DNA
gaccctggaaaagctgatgagttttagagctagaaatagcaagttaaaataaggctagtccgttatcaacttgaaaaagtggcaccgagtcggtgcgcggcggcggcggtggcggctgttgctgctgctgctggaaggacttgagggactcgaaggctttcatcagcttttcca

SEQ ID NO: 470 - DNA, 175 bp, synthetic construct
  source 1..175: mol_type = other DNA
gaccctggaaaagctgatgagttttagagctagaaatagcaagttaaaataaggctagtccgttatcaacttgaaaaagtggcaccgagtcggtgcgcggcggcggcggtggcggctgttgctgctgctgctggaaggacttgagggactcgaaggctttcatcagcttttccag

SEQ ID NO: 471 - DNA, 176 bp, synthetic construct
  source 1..176: mol_type = other DNA
gaccctggaaaagctgatgagttttagagctagaaatagcaagttaaaataaggctagtccgttatcaacttgaaaaagtggcaccgagtcggtgcgcggcggcggcggtggcggctgttgctgctgctgctggaaggacttgagggactcgaaggctttcatcagcttttccagg

SEQ ID NO: 472 - DNA, 177 bp, synthetic construct
  source 1..177: mol_type = other DNA
gaccctggaaaagctgatgagttttagagctagaaatagcaagttaaaataaggctagtccgttatcaacttgaaaaagtggcaccgagtcggtgcgcggcggcggcggcggcggtggcggctgttgctgctgctgctggaaggacttgagggactcgaaggctttcatcagctttt

SEQ ID NO: 473 - DNA, 178 bp, synthetic construct
  source 1..178: mol_type = other DNA
gaccctggaaaagctgatgagttttagagctagaaatagcaagttaaaataaggctagtccgttatcaacttgaaaaagtggcaccgagtcggtgcgcggcggcggcggcggcggtggcggctgttgctgctgctgctggaaggacttgagggactcgaaggctttcatcagcttttc

SEQ ID NO: 474 - DNA, 180 bp, synthetic construct
  source 1..180: mol_type = other DNA
gaccctggaaaagctgatgagttttagagctagaaatagcaagttaaaataaggctagtccgttatcaacttgaaaaagtggcaccgagtcggtgcgcggcggcggcggcggcggtggcggctgttgctgctgctgctggaaggacttgagggactcgaaggctttcatcagcttttcca

SEQ ID NO: 475 - DNA, 181 bp, synthetic construct
  source 1..181: mol_type = other DNA
gaccctggaaaagctgatgagttttagagctagaaatagcaagttaaaataaggctagtccgttatcaacttgaaaaagtggcaccgagtcggtgcgcggcggcggcggcggcggtggcggctgttgctgctgctgctggaaggacttgagggactcgaaggctttcatcagcttttccag

SEQ ID NO: 476 - DNA, 180 bp, synthetic construct
  source 1..180: mol_type = other DNA
gaccctggaaaagctgatgagttttagagctagaaatagcaagttaaaataaggctagtccgttatcaacttgaaaaagtggcaccgagtcggtgcggcggcggcggcggcggcggcggtggcggctgttgctgctgctgctggaaggacttgagggactcgaaggctttcatcagcttt

SEQ ID NO: 477 - DNA, 182 bp, synthetic construct
  source 1..182: mol_type = other DNA
gaccctggaaaagctgatgagttttagagctagaaatagcaagttaaaataaggctagtccgttatcaacttgaaaaagtggcaccgagtcggtgcggcggcggcggcggcggcggcggtggcggctgttgctgctgctgctggaaggacttgagggactcgaaggctttcatcagcttttc

SEQ ID NO: 478 - DNA, 223 bp, synthetic construct
  source 1..223: mol_type = other DNA
gaccctggaaaagctgatgagttttagagctagaaatagcaagttaaaataaggctagtccgttatcaacttgaaaaagtggcaccgagtcggtgcgcggcggcggcggcggcggtggcggctgttgctgctgctgctggaaggacttgagggactcgaaggctttcatcagcttttccaatcactcgcggttctatctagttacgcgttaaaccaactagaa

SEQ ID NO: 479 - DNA, 223 bp, synthetic construct
  source 1..223: mol_type = other DNA
gaccctggaaaagctgatgagttttagagctagaaatagcaagttaaaataaggctagtccgttatcaacttgaaaaagtggcaccgagtcggtgcgcggcggcggcggcggcggtggcggctgttgctgctgctgctggaaggacttgagggactcgaaggctttcatcagcttttcgtataatacgcggttctatctagttacgcgttaaaccaactagaa

SEQ ID NO: 480 - DNA, 232 bp, synthetic construct
  source 1..232: mol_type = other DNA
gaccctggaaaagctgatgagttttagagctagaaatagcaagttaaaataaggctagtccgttatcaacttgaaaaagtggcaccgagtcggtgcgaggaggcggcggcggcggcggcggcggtggcggctgttgctgctgctgctggaaggacttgagggactcgaaggctttcatcagcttttcattaaattcgcggttctatctagttacgcgttaaaccaactagaa

SEQ ID NO: 481 - DNA, 237 bp, synthetic construct
  source 1..237: mol_type = other DNA
gaccctggaaaagctgatgagttttagagctagaaatagcaagttaaaataaggctagtccgttatcaacttgaaaaagtggcaccgagtcggtgcgcggcggcggcggcggcggtggcggctgttgctgctgctgctggaaggacttgagggactcgaaggctttcatcagcttttccaatcactgggtcaggagccccccccctgaacccaggataaccctcaaagtcggggggc

SEQ ID NO: 482 - DNA, 246 bp, synthetic construct
  source 1..246: mol_type = other DNA
gaccctggaaaagctgatgagttttagagctagaaatagcaagttaaaataaggctagtccgttatcaacttgaaaaagtggcaccgagtcggtgcgaggaggcggcggcggcggcggcggcggtggcggctgttgctgctgctgctggaaggacttgagggactcgaaggctttcatcagcttttccaatcactgggtcaggagccccccccctgaacccaggataaccctcaaagtcggggggc

SEQ ID NO: 483 - DNA, 217 bp, synthetic construct
  source 1..217: mol_type = other DNA
gaccctggaaaagctgatgagttttagagctagaaatagcaagttaaaataaggctagtccgttatcaacttgaaaaagtggcaccgagtcggtgcgcggcggcggcggtggcggctgttgctgctgctgctggaaggacttgagggactcgaaggctttcatcagcttttccaatcactcgcggttctatctagttacgcgttaaaccaactagaa

SEQ ID NO: 484 - DNA, 227 bp, synthetic construct
  source 1..227: mol_type = other DNA
gaccctggaaaagctgatgagttttagagctagaaatagcaagttaaaataaggctagtccgttatcaacttgaaaaagtggcaccgagtcggtgcggcggcggcggcggcggcggcggtggcggctgttgctgctgctgctggaaggacttgagggactcgaaggctttcatcagcttttccaatcactcgcggttctatctagttacgcgttaaaccaactagaa

SEQ ID NO: 485 - DNA, 232 bp, synthetic construct
  source 1..232: mol_type = other DNA
gaccctggaaaagctgatgagttttagagctagaaatagcaagttaaaataaggctagtccgttatcaacttgaaaaagtggcaccgagtcggtgcgaggaggcggcggcggcggcggcggcggtggcggctgttgctgctgctgctggaaggacttgagggactcgaaggctttcatcagcttttccaatcactcgcggttctatctagttacgcgttaaaccaactagaa

SEQ ID NO: 486 - DNA, 253 bp, synthetic construct
  source 1..253: mol_type = other DNA
gaccctggaaaagctgatgagttttagagctagaaatagcaagttaaaataaggctagtccgttatcaacttgaaaaagtggcaccgagtcggtgcgcggcggcggctgaggaagctgaggaggcggcggcggcggcggcggcggtggcggctgttgctgctgctgctggaaggacttgagggactcgaaggctttcatcagcttttccaatcactcgcggttctatctagttacgcgttaaaccaactagaa

SEQ ID NO: 487 - DNA, 236 bp, synthetic construct
  source 1..236: mol_type = other DNA
gaccctggaaaagctgatgagttttagagctagaaatagcaagttaaaataaggctagtccgttatcaacttgaaaaagtggcaccgagtcggtgcagctgaggaggcggcggcggcggcggcggcggtggcggctgttgctgctgctgctggaaggacttgagggactcgaaggctttcatcagcttttccaatcactcgcggttctatctagttacgcgttaaaccaactagaa

SEQ ID NO: 488 - DNA, 256 bp, synthetic construct
  source 1..256: mol_type = other DNA
gtgaggaagctgaggaggcgggttttagagctagaaatagcaagttaaaataaggctagtccgttatcaacttgaaaaagtggcaccgagtcggtgcagaccgccatggcgaccctggaaaagctgatgaaagccttcgagtccctcaagtccttccagcagcagcagcaacagccgccaccgccgccgccgccgccgccgcctcctcagccaatcactcgcggttctatctagttacgcgttaaaccaactagaa

SEQ ID NO: 489 - DNA, 234 bp, synthetic construct
  source 1..234: mol_type = other DNA
ggcggcggcggcggcggtgggttttagagctagaaatagcaagttaaaataaggctagtccgttatcaacttgaaaaagtggcaccgagtcggtgcagaccgccatggcgaccctggaaaagctgatgaaagccttcgagtccctcaagtccttccagcagcagcagcaacagccgccaccgccgccgccaatcactcgcggttctatctagttacgcgttaaaccaactagaa

SEQ ID NO: 490 - DNA, 204 bp, synthetic construct
  source 1..204: mol_type = other DNA
gaccctggaaaagctgatgagttttagagctagaaatagcaagttaaaataaggctagtccgttatcaacttgaaaaagtggcaccgagtcggtgctggcggctgttgctgctgctgctggaaggacttgagggactcgaaggctttcatcagcttttccaatcactcgcggttctatctagttacgcgttaaaccaactagaa

SEQ ID NO: 491 - DNA, 226 bp, synthetic construct
  source 1..226: mol_type = other DNA
gtgaggaagctgaggaggcgggttttagagctagaaatagcaagttaaaataaggctagtccgttatcaacttgaaaaagtggcaccgagtcggtgctgaaagccttcgagtccctcaagtccttccagcagcagcagcaacagccgccaccgccgccgccgccgccgccgcctcctcagccaatcactcgcggttctatctagttacgcgttaaaccaactagaa

SEQ ID NO: 492 - DNA, 204 bp, synthetic construct
  source 1..204: mol_type = other DNA
ggcggcggcggcggcggtgggttttagagctagaaatagcaagttaaaataaggctagtccgttatcaacttgaaaaagtggcaccgagtcggtgctgaaagccttcgagtccctcaagtccttccagcagcagcagcaacagccgccaccgccgccgccaatcactcgcggttctatctagttacgcgttaaaccaactagaa

SEQ ID NO: 493 - DNA, 225 bp, synthetic construct
  source 1..225: mol_type = other DNA
gaccctggaaaagctgatgagttttagagctagaaatagcaagttaaaataaggctagtccgttatcaacttgaaaaagtggcaccgagtcggtgcggtggagggggcggtggagggggtggaggctgttgctgttgctgctgaaatgatttcagtgattcaaatgctttcatcagctttcaatcactcgcggttctatctagttacgcgttaaaccaactagaa

SEQ ID NO: 494 - DNA, 227 bp, synthetic construct
  source 1..227: mol_type = other DNA
gaccctggaaaagctgatgagttttagagctagaaatagcaagttaaaataaggctagtccgttatcaacttgaaaaagtggcaccgagtcggtgcggtggagggggcggtggagggggtggaggctgttgctgttgctgctgaaatgatttcagtgattcaaatgctttcatcagcttttccaatcactcgcggttctatctagttacgcgttaaaccaactagaa

SEQ ID NO: 495 - DNA, 202 bp, synthetic construct
  source 1..202: mol_type = other DNA
gaccctggaaaagctgatgagttttagagctagaaatagcaagttaaaataaggctagtccgttatcaacttgaaaaagtggcaccgagtcggtgctggaggctgttgctgttgctgctgaaatgatttcagtgattcaaatgctttcatcagctttcaatcactcgcggttctatctagttacgcgttaaaccaactagaa

SEQ ID NO: 496 - DNA, 204 bp, synthetic construct
  source 1..204: mol_type = other DNA
gaccctggaaaagctgatgagttttagagctagaaatagcaagttaaaataaggctagtccgttatcaacttgaaaaagtggcaccgagtcggtgctggaggctgttgctgttgctgctgaaatgatttcagtgattcaaatgctttcatcagcttttccaatcactcgcggttctatctagttacgcgttaaaccaactagaa

SEQ ID NO: 497 - DNA, 224 bp, synthetic construct
  source 1..224: mol_type = other DNA
gtgaggaagctgaggaggcgggttttagagctagaaatagcaagttaaaataaggctagtccgttatcaacttgaaaaagtggcaccgagtcggtgctgaaagcatttgaatcactgaaatcatttcagcagcaacagcaacagcctccaccccctccaccgccccctccacctcctcacaatcactcgcggttctatctagttacgcgttaaaccaactagaa

SEQ ID NO: 498 - DNA, 226 bp, synthetic construct
  source 1..226: mol_type = other DNA
gtgaggaagctgaggaggcgggttttagagctagaaatagcaagttaaaataaggctagtccgttatcaacttgaaaaagtggcaccgagtcggtgctgaaagcatttgaatcactgaaatcatttcagcagcaacagcaacagcctccaccccctccaccgccccctccacctcctcagccaatcactcgcggttctatctagttacgcgttaaaccaactagaa

SEQ ID NO: 499 - DNA, 202 bp, synthetic construct
  source 1..202: mol_type = other DNA
ggcggcggcggcggcggtgggttttagagctagaaatagcaagttaaaataaggctagtccgttatcaacttgaaaaagtggcaccgagtcggtgctgaaagcatttgaatcactgaaatcatttcagcagcaacagcaacagcctccaccgccgcccaatcactcgcggttctatctagttacgcgttaaaccaactagaa

SEQ ID NO: 500 - DNA, 204 bp, synthetic construct
  source 1..204: mol_type = other DNA
ggcggcggcggcggcggtgggttttagagctagaaatagcaagttaaaataaggctagtccgttatcaacttgaaaaagtggcaccgagtcggtgctgaaagcatttgaatcactgaaatcatttcagcagcaacagcaacagcctccaccgccgccgccaatcactcgcggttctatctagttacgcgttaaaccaactagaa

SEQ ID NO: 501 - DNA, 217 bp, synthetic construct
  source 1..217: mol_type = other DNA
gaccctggaaaagctgatgagttttagagctagaaatagcaagttaaaataaggctagtccgttatcaacttgaaaaagtggcaccgagtcggtgcgcggcggcggcggcggcggtggcggctgttgctgctggaaggacttgagggactcgaaggctttcatcagcttttccaatcactcgcggttctatctagttacgcgttaaaccaactagaa

SEQ ID NO: 502 - DNA, 220 bp, synthetic construct
  source 1..220: mol_type = other DNA
gaccctggaaaagctgatgagttttagagctagaaatagcaagttaaaataaggctagtccgttatcaacttgaaaaagtggcaccgagtcggtgcgcggcggcggcggcggcggtggcggctgttgctgctgctggaaggacttgagggactcgaaggctttcatcagcttttccaatcactcgcggttctatctagttacgcgttaaaccaactagaa

SEQ ID NO: 503 - DNA, 226 bp, synthetic construct
  source 1..226: mol_type = other DNA
gaccctggaaaagctgatgagttttagagctagaaatagcaagttaaaataaggctagtccgttatcaacttgaaaaagtggcaccgagtcggtgcgcggcggcggcggcggcggtggcggctgttgctgctgctgctgctggaaggacttgagggactcgaaggctttcatcagcttttccaatcactcgcggttctatctagttacgcgttaaaccaactagaa

SEQ ID NO: 504 - DNA, 229 bp, synthetic construct
  source 1..229: mol_type = other DNA
gaccctggaaaagctgatgagttttagagctagaaatagcaagttaaaataaggctagtccgttatcaacttgaaaaagtggcaccgagtcggtgcgcggcggcggcggcggcggtggcggctgttgctgctgctgctgctgctggaaggacttgagggactcgaaggctttcatcagcttttccaatcactcgcggttctatctagttacgcgttaaaccaactagaa

SEQ ID NO: 505 - DNA, 229 bp, synthetic construct
  source 1..229: mol_type = other DNA
gaccctggaaaagctgatgagttttagagctagaaatagcaagttaaaataaggctagtccgttatcaacttgaaaaagtggcaccgagtcggtgcgcggcggcggcggcggcggtggcggctgttgctgctgttgctgctgctggaaggacttgagggactcgaaggctttcatcagcttttccaatcactcgcggttctatctagttacgcgttaaaccaactagaa

SEQ ID NO: 506 - DNA, 232 bp, synthetic construct
  source 1..232: mol_type = other DNA
gaccctggaaaagctgatgagttttagagctagaaatagcaagttaaaataaggctagtccgttatcaacttgaaaaagtggcaccgagtcggtgcgcggcggcggcggcggcggtggcggctgttgctgctgctgctgctgctgctggaaggacttgagggactcgaaggctttcatcagcttttccaatcactcgcggttctatctagttacgcgttaaaccaactagaa

SEQ ID NO: 507 - DNA, 235 bp, synthetic construct
  source 1..235: mol_type = other DNA
gaccctggaaaagctgatgagttttagagctagaaatagcaagttaaaataaggctagtccgttatcaacttgaaaaagtggcaccgagtcggtgcgcggcggcggcggcggcggtggcggctgttgctgctgctgctgctgctgctgctggaaggacttgagggactcgaaggctttcatcagcttttccaatcactcgcggttctatctagttacgcgttaaaccaactagaa

SEQ ID NO: 508 - DNA, 235 bp, synthetic construct
  source 1..235: mol_type = other DNA
gaccctggaaaagctgatgagttttagagctagaaatagcaagttaaaataaggctagtccgttatcaacttgaaaaagtggcaccgagtcggtgcgcggcggcggcggcggcggtggcggctgttgctgctgctgttgctgctgctgctggaaggacttgagggactcgaaggctttcatcagcttttccaatcactcgcggttctatctagttacgcgttaaaccaactagaa

SEQ ID NO: 509 - DNA, 238 bp, synthetic construct
  source 1..238: mol_type = other DNA
gaccctggaaaagctgatgagttttagagctagaaatagcaagttaaaataaggctagtccgttatcaacttgaaaaagtggcaccgagtcggtgcgcggcggcggcggcggcggtggcggctgttgctgctgctgctgctgctgctgctgctggaaggacttgagggactcgaaggctttcatcagcttttccaatcactcgcggttctatctagttacgcgttaaaccaactagaa

SEQ ID NO: 510 - DNA, 226 bp, synthetic construct
  source 1..226: mol_type = other DNA
gaccctggaaaagctgatgagttttagagctagaaatagcaagttaaaataaggctagtccgttatcaacttgaaaaagtggcaccgagtcggtgcgaggaggcggcggcggcggcggcggcggtggcggctgttgctgctggaaggacttgagggactcgaaggctttcatcagcttttccaatcactcgcggttctatctagttacgcgttaaaccaactagaa

SEQ ID NO: 511 - DNA, 229 bp, synthetic construct
  source 1..229: mol_type = other DNA
gaccctggaaaagctgatgagttttagagctagaaatagcaagttaaaataaggctagtccgttatcaacttgaaaaagtggcaccgagtcggtgcgaggaggcggcggcggcggcggcggcggtggcggctgttgctgctgctggaaggacttgagggactcgaaggctttcatcagcttttccaatcactcgcggttctatctagttacgcgttaaaccaactagaa

SEQ ID NO: 512 - DNA, 235 bp, synthetic construct
  source 1..235: mol_type = other DNA
gaccctggaaaagctgatgagttttagagctagaaatagcaagttaaaataaggctagtccgttatcaacttgaaaaagtggcaccgagtcggtgcgaggaggcggcggcggcggcggcggcggtggcggctgttgctgctgctgctgctggaaggacttgagggactcgaaggctttcatcagcttttccaatcactcgcggttctatctagttacgcgttaaaccaactagaa

SEQ ID NO: 513 - DNA, 238 bp, synthetic construct
  source 1..238: mol_type = other DNA
gaccctggaaaagctgatgagttttagagctagaaatagcaagttaaaataaggctagtccgttatcaacttgaaaaagtggcaccgagtcggtgcgaggaggcggcggcggcggcggcggcggtggcggctgttgctgctgctgctgctgctggaaggacttgagggactcgaaggctttcatcagcttttccaatcactcgcggttctatctagttacgcgttaaaccaactagaa

SEQ ID NO: 514 - DNA, 241 bp, synthetic construct
  source 1..241: mol_type = other DNA
gaccctggaaaagctgatgagttttagagctagaaatagcaagttaaaataaggctagtccgttatcaacttgaaaaagtggcaccgagtcggtgcgaggaggcggcggcggcggcggcggcggtggcggctgttgctgctgctgctgctgctgctggaaggacttgagggactcgaaggctttcatcagcttttccaatcactcgcggttctatctagttacgcgttaaaccaactagaa

SEQ ID NO: 515 - DNA, 244 bp, synthetic construct
  source 1..244: mol_type = other DNA
gaccctggaaaagctgatgagttttagagctagaaatagcaagttaaaataaggctagtccgttatcaacttgaaaaagtggcaccgagtcggtgcgaggaggcggcggcggcggcggcggcggtggcggctgttgctgctgctgctgctgctgctgctggaaggacttgagggactcgaaggctttcatcagcttttccaatcactcgcggttctatctagttacgcgttaaaccaactagaa

SEQ ID NO: 516 - DNA, 247 bp, synthetic construct
  source 1..247: mol_type = other DNA
gaccctggaaaagctgatgagttttagagctagaaatagcaagttaaaataaggctagtccgttatcaacttgaaaaagtggcaccgagtcggtgcgaggaggcggcggcggcggcggcggcggtggcggctgttgctgctgctgctgctgctgctgctgctggaaggacttgagggactcgaaggctttcatcagcttttccaatcactcgcggttctatctagttacgcgttaaaccaactagaa

SEQ ID NO: 517 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctggaaggacttgagggactcgaaggctttcatcagcttttccaatcactcgcggttctatctagttacgcgttaaaccaactagaa

SEQ ID NO: 518 - DNA, 227 bp, synthetic construct
  source 1..227: mol_type = other DNA
gaccctggaaaagctgatgagtttaagagctagaaatagcaagtttaaataaggctagtccgttatcaacttgaaaaagtggcaccgagtcggtgcggcggcggcggcggcggcggcggtggcggctgttgctgctgctgctggaaggacttgagggactcgaaggctttcatcagcttttccaatcactcgcggttctatctagttacgcgttaaaccaactagaa

SEQ ID NO: 519 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctggaaggacttgagggactcgaaggctttcatcagcttttccaatcactcgcggttctatctagttacgcgttaaaccaactagaa

SEQ ID NO: 520 - DNA, 238 bp, synthetic construct
  source 1..238: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctgctgctgctgctgctggaaggacttgagggactcgaaggctttcatcagcttttccaatcactcgcggttctatctagttacgcgttaaaccaactagaa

SEQ ID NO: 521 - DNA, 247 bp, synthetic construct
  source 1..247: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctgctgctgctgctgctggaaggacttgagggactcgaaggctttcatcagcttttccaatcactcgcggttctatctagttacgcgttaaaccaactagaa

SEQ ID NO: 522 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgttgctgctggaaggacttgagggactcgaaggctttcatcagcttttccaatcactcgcggttctatctagttacgcgttaaaccaactagaa

SEQ ID NO: 523 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgttgctgttggaaggacttgagggactcgaaggctttcatcagcttttccaatcactcgcggttctatctagttacgcgttaaaccaactagaa

SEQ ID NO: 524 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgttgttgttgttggaaggacttgagggactcgaaggctttcatcagcttttccaatcactcgcggttctatctagttacgcgttaaaccaactagaa

SEQ ID NO: 525 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgttgctgctggaaggacttcagggactcgaaggctttcatcagcttttccaatcactcgcggttctatctagttacgcgttaaaccaactagaa

SEQ ID NO: 526 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgttgctgttggaaggacttcagggactcgaaggctttcatcagcttttccaatcactcgcggttctatctagttacgcgttaaaccaactagaa

SEQ ID NO: 527 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgttgttgttgttggaaggacttcagggactcgaaggctttcatcagcttttccaatcactcgcggttctatctagttacgcgttaaaccaactagaa

SEQ ID NO: 528 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgttgttgttgttgaaacgatttcagcgattcaaacgctttcatcagcttttccaatcactcgcggttctatctagttacgcgttaaaccaactagaa

SEQ ID NO: 529 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctggaaggacttcagggactcgaaggctttcatcagcttttccaatcactcgcggttctatctagttacgcgttaaaccaactagaa

SEQ ID NO: 530 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctggaaggacttcagggattcgaaggctttcatcagcttttccaatcactcgcggttctatctagttacgcgttaaaccaactagaa

SEQ ID NO: 531 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctggaaggacttcagggactcgaaggctttcatcagcttttccaatcactcgcggttctatctagttacgcgttaaaccaactagaa

SEQ ID NO: 532 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctgaaaggacttcagggattcgaaggctttcatcagcttttccaatcactcgcggttctatctagttacgcgttaaaccaactagaa

SEQ ID NO: 533 - DNA, 238 bp, synthetic construct
  source 1..238: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctgctgctgctgctgctggaaggacttcagggattcgaaggctttcatcagcttttccaatcactcgcggttctatctagttacgcgttaaaccaactagaa

SEQ ID NO: 534 - DNA, 238 bp, synthetic construct
  source 1..238: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgttgctgttgctgttgctgttgctgttggaaggacttgagggactcgaaggctttcatcagcttttccaatcactcgcggttctatctagttacgcgttaaaccaactagaa

SEQ ID NO: 535 - DNA, 238 bp, synthetic construct
  source 1..238: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgttgctgttgctgttgctgttgctgttggaaggacttcagggattcgaaggctttcatcagcttttccaatcactcgcggttctatctagttacgcgttaaaccaactagaa

SEQ ID NO: 536 - DNA, 247 bp, synthetic construct
  source 1..247: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctgctgctgctgctgctggaaggacttcagggattcgaaggctttcatcagcttttccaatcactcgcggttctatctagttacgcgttaaaccaactagaa

SEQ ID NO: 537 - DNA, 247 bp, synthetic construct
  source 1..247: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgttgctgttgctgttgctgttgctgttggaaggacttgagggactcgaaggctttcatcagcttttccaatcactcgcggttctatctagttacgcgttaaaccaactagaa

SEQ ID NO: 538 - DNA, 247 bp, synthetic construct
  source 1..247: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgttgctgttgctgttgctgttgctgttggaaggacttcagggattcgaaggctttcatcagcttttccaatcactcgcggttctatctagttacgcgttaaaccaactagaa

SEQ ID NO: 539 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctggaaggacttgagggactcgaaggcgttcatcagcttttccaatcactcgcggttctatctagttacgcgttaaaccaactagaa

SEQ ID NO: 540 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctggaaggacttgagggactcgaagggcttcatcagcttttccaatcactcgcggttctatctagttacgcgttaaaccaactagaa

SEQ ID NO: 541 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctggaaggacttgagggactcgaaggggttcatcagcttttccaatcactcgcggttctatctagttacgcgttaaaccaactagaa

SEQ ID NO: 542 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctggaacgacttcagcgactcgaaggctttcatcagcttttccaatcactcgcggttctatctagttacgcgttaaaccaactagaa

SEQ ID NO: 543 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctggaacgacttgagggactcgaaggctttcatcagcttttccaatcactcgcggttctatctagttacgcgttaaaccaactagaa

SEQ ID NO: 544 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctggaaggacttgagcgactcgaaggctttcatcagcttttccaatcactcgcggttctatctagttacgcgttaaaccaactagaa

SEQ ID NO: 545 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctggaaggatttgagggactcgaaggctttcatcagcttttccaatcactcgcggttctatctagttacgcgttaaaccaactagaa

SEQ ID NO: 546 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctgaaaggacttgagggactcgaaggctttcatcagcttttccaatcactcgcggttctatctagttacgcgttaaaccaactagaa

SEQ ID NO: 547 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctggaaggacttgagggattcgaaggctttcatcagcttttccaatcactcgcggttctatctagttacgcgttaaaccaactagaa

SEQ ID NO: 548 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctggaaggacttgagggactcaaaggctttcatcagcttttccaatcactcgcggttctatctagttacgcgttaaaccaactagaa

SEQ ID NO: 549 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctggaaggacttgagggactcgaacgctttcatcagcttttccaatcactcgcggttctatctagttacgcgttaaaccaactagaa

SEQ ID NO: 550 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctggaaggatttcagggactcgaaggctttcatcagcttttccaatcactcgcggttctatctagttacgcgttaaaccaactagaa

SEQ ID NO: 551 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctgaaaggacttcagggactcgaaggctttcatcagcttttccaatcactcgcggttctatctagttacgcgttaaaccaactagaa

SEQ ID NO: 552 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctggaaggacttcagggactcaaaggctttcatcagcttttccaatcactcgcggttctatctagttacgcgttaaaccaactagaa

SEQ ID NO: 553 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctggaaggacttcagggactcgaacgctttcatcagcttttccaatcactcgcggttctatctagttacgcgttaaaccaactagaa

SEQ ID NO: 554 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctggaaggacttcagcgactcgaaggctttcatcagcttttccaatcactcgcggttctatctagttacgcgttaaaccaactagaa

SEQ ID NO: 555 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctggaacgacttcagggactcgaaggctttcatcagcttttccaatcactcgcggttctatctagttacgcgttaaaccaactagaa

SEQ ID NO: 556 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctgaaaggatttgagggactcgaaggctttcatcagcttttccaatcactcgcggttctatctagttacgcgttaaaccaactagaa

SEQ ID NO: 557 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctggaaggatttgagggattcgaaggctttcatcagcttttccaatcactcgcggttctatctagttacgcgttaaaccaactagaa

SEQ ID NO: 558 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctggaaggatttgagggactcaaaggctttcatcagcttttccaatcactcgcggttctatctagttacgcgttaaaccaactagaa

SEQ ID NO: 559 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctggaaggatttgagggactcgaacgctttcatcagcttttccaatcactcgcggttctatctagttacgcgttaaaccaactagaa

SEQ ID NO: 560 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctgaaaggacttgagggattcgaaggctttcatcagcttttccaatcactcgcggttctatctagttacgcgttaaaccaactagaa

SEQ ID NO: 561 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctgaaaggacttgagggactcaaaggctttcatcagcttttccaatcactcgcggttctatctagttacgcgttaaaccaactagaa

SEQ ID NO: 562 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctgaaaggacttgagggactcgaacgctttcatcagcttttccaatcactcgcggttctatctagttacgcgttaaaccaactagaa

SEQ ID NO: 563 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctggaaggacttgagggattcaaaggctttcatcagcttttccaatcactcgcggttctatctagttacgcgttaaaccaactagaa

SEQ ID NO: 564 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctggaaggacttgagggattcgaacgctttcatcagcttttccaatcactcgcggttctatctagttacgcgttaaaccaactagaa

SEQ ID NO: 565 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctggaaggacttgagggactcaaacgctttcatcagcttttccaatcactcgcggttctatctagttacgcgttaaaccaactagaa

SEQ ID NO: 566 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctggaaggatttgagcgactcgaaggctttcatcagcttttccaatcactcgcggttctatctagttacgcgttaaaccaactagaa

SEQ ID NO: 567 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctgaaaggacttgagcgactcgaaggctttcatcagcttttccaatcactcgcggttctatctagttacgcgttaaaccaactagaa

SEQ ID NO: 568 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctggaaggacttgagcgattcgaaggctttcatcagcttttccaatcactcgcggttctatctagttacgcgttaaaccaactagaa

SEQ ID NO: 569 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctggaaggacttgagcgactcaaaggctttcatcagcttttccaatcactcgcggttctatctagttacgcgttaaaccaactagaa

SEQ ID NO: 570 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctggaaggacttgagcgactcgaacgctttcatcagcttttccaatcactcgcggttctatctagttacgcgttaaaccaactagaa

SEQ ID NO: 571 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctggaaggacttcagcgattcgaaggctttcatcagcttttccaatcactcgcggttctatctagttacgcgttaaaccaactagaa

SEQ ID NO: 572 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctggaaggacttcagcgactcgaacgctttcatcagcttttccaatcactcgcggttctatctagttacgcgttaaaccaactagaa

SEQ ID NO: 573 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctggaaggatttcagcgactcgaaggctttcatcagcttttccaatcactcgcggttctatctagttacgcgttaaaccaactagaa

SEQ ID NO: 574 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctgaaaggacttcagcgactcgaaggctttcatcagcttttccaatcactcgcggttctatctagttacgcgttaaaccaactagaa

SEQ ID NO: 575 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctggaaggacttcagcgactcaaaggctttcatcagcttttccaatcactcgcggttctatctagttacgcgttaaaccaactagaa

SEQ ID NO: 576 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctgaaaggatttgagggattcgaaggctttcatcagcttttccaatcactcgcggttctatctagttacgcgttaaaccaactagaa

SEQ ID NO: 577 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctgaaaggatttgagggactcaaaggctttcatcagcttttccaatcactcgcggttctatctagttacgcgttaaaccaactagaa

SEQ ID NO: 578 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctgaaaggatttgagggactcgaacgctttcatcagcttttccaatcactcgcggttctatctagttacgcgttaaaccaactagaa

SEQ ID NO: 579 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctgaaaggatttcagggactcgaaggctttcatcagcttttccaatcactcgcggttctatctagttacgcgttaaaccaactagaa

SEQ ID NO: 580 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctgaaaggacttgagggattcaaaggctttcatcagcttttccaatcactcgcggttctatctagttacgcgttaaaccaactagaa

SEQ ID NO: 581 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctgaaaggacttgagggattcgaacgctttcatcagcttttccaatcactcgcggttctatctagttacgcgttaaaccaactagaa

SEQ ID NO: 582 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctggaaggacttcagggactcaaacgctttcatcagcttttccaatcactcgcggttctatctagttacgcgttaaaccaactagaa

SEQ ID NO: 583 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctggaaggatttgagggattcgaacgctttcatcagcttttccaatcactcgcggttctatctagttacgcgttaaaccaactagaa

SEQ ID NO: 584 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctggaaggatttcagggattcgaaggctttcatcagcttttccaatcactcgcggttctatctagttacgcgttaaaccaactagaa

SEQ ID NO: 585 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctggaaggatttgagggactcaaacgctttcatcagcttttccaatcactcgcggttctatctagttacgcgttaaaccaactagaa

SEQ ID NO: 586 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctggaaggatttgagcgattcgaaggctttcatcagcttttccaatcactcgcggttctatctagttacgcgttaaaccaactagaa

SEQ ID NO: 587 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctgaaaggacttgagcgattcgaaggctttcatcagcttttccaatcactcgcggttctatctagttacgcgttaaaccaactagaa

SEQ ID NO: 588 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctggaaggacttgagcgattcaaaggctttcatcagcttttccaatcactcgcggttctatctagttacgcgttaaaccaactagaa

SEQ ID NO: 589 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctggaaggatttgagcgactcgaacgctttcatcagcttttccaatcactcgcggttctatctagttacgcgttaaaccaactagaa

SEQ ID NO: 590 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctgaaaggacttgagcgactcgaacgctttcatcagcttttccaatcactcgcggttctatctagttacgcgttaaaccaactagaa

SEQ ID NO: 591 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctggaaggacttgagcgattcgaacgctttcatcagcttttccaatcactcgcggttctatctagttacgcgttaaaccaactagaa

SEQ ID NO: 592 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctggaaggacttgagcgactcaaacgctttcatcagcttttccaatcactcgcggttctatctagttacgcgttaaaccaactagaa

SEQ ID NO: 593 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctgaaaggatttgagcgactcgaaggctttcatcagcttttccaatcactcgcggttctatctagttacgcgttaaaccaactagaa

SEQ ID NO: 594 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctggaaggatttgagcgactcaaaggctttcatcagcttttccaatcactcgcggttctatctagttacgcgttaaaccaactagaa

SEQ ID NO: 595 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctggaaggatttcagggactcgaacgctttcatcagcttttccaatcactcgcggttctatctagttacgcgttaaaccaactagaa

SEQ ID NO: 596 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctgaaaggacttcagggactcgaacgctttcatcagcttttccaatcactcgcggttctatctagttacgcgttaaaccaactagaa

SEQ ID NO: 597 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctggaaggacttcagggattcgaacgctttcatcagcttttccaatcactcgcggttctatctagttacgcgttaaaccaactagaa

SEQ ID NO: 598 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctgaaaggacttcagggactcaaaggctttcatcagcttttccaatcactcgcggttctatctagttacgcgttaaaccaactagaa

SEQ ID NO: 599 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctgaaaggacttgagcgactcaaaggctttcatcagcttttccaatcactcgcggttctatctagttacgcgttaaaccaactagaa

SEQ ID NO: 600 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctgaaaggacttgagggactcaaacgctttcatcagcttttccaatcactcgcggttctatctagttacgcgttaaaccaactagaa

SEQ ID NO: 601 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctggaaggacttcagcgattcgaacgctttcatcagcttttccaatcactcgcggttctatctagttacgcgttaaaccaactagaa

SEQ ID NO: 602 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctggaaggatttcagcgactcgaacgctttcatcagcttttccaatcactcgcggttctatctagttacgcgttaaaccaactagaa

SEQ ID NO: 603 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctgaaaggacttcagcgactcgaacgctttcatcagcttttccaatcactcgcggttctatctagttacgcgttaaaccaactagaa

SEQ ID NO: 604 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctggaaggacttcagcgactcaaacgctttcatcagcttttccaatcactcgcggttctatctagttacgcgttaaaccaactagaa

SEQ ID NO: 605 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctgaaaggatttgagcgactcgaacgctttcatcagcttttccaatcactcgcggttctatctagttacgcgttaaaccaactagaa

SEQ ID NO: 606 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctggaaggatttgagcgattcgaacgctttcatcagcttttccaatcactcgcggttctatctagttacgcgttaaaccaactagaa

SEQ ID NO: 607 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctgaaaggatttgagggattcgaacgctttcatcagcttttccaatcactcgcggttctatctagttacgcgttaaaccaactagaa

SEQ ID NO: 608 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctgaaaggatttgagggactcaaacgctttcatcagcttttccaatcactcgcggttctatctagttacgcgttaaaccaactagaa

SEQ ID NO: 609 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctgaaaggatttcagggactcgaacgctttcatcagcttttccaatcactcgcggttctatctagttacgcgttaaaccaactagaa

SEQ ID NO: 610 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctgaaaggatttgagggattcaaaggctttcatcagcttttccaatcactcgcggttctatctagttacgcgttaaaccaactagaa

SEQ ID NO: 611 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctgaaaggatttcagggattcgaaggctttcatcagcttttccaatcactcgcggttctatctagttacgcgttaaaccaactagaa

SEQ ID NO: 612 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctgaaaggatttgagcgattcgaaggctttcatcagcttttccaatcactcgcggttctatctagttacgcgttaaaccaactagaa

SEQ ID NO: 613 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctgaaaggacttcagggattcaaaggctttcatcagcttttccaatcactcgcggttctatctagttacgcgttaaaccaactagaa

SEQ ID NO: 614 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctgaaaggacttgagcgattcaaaggctttcatcagcttttccaatcactcgcggttctatctagttacgcgttaaaccaactagaa

SEQ ID NO: 615 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctgaaaggacttgagggattcaaacgctttcatcagcttttccaatcactcgcggttctatctagttacgcgttaaaccaactagaa

SEQ ID NO: 616 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctgaaaggatttcagcgactcgaaggctttcatcagcttttccaatcactcgcggttctatctagttacgcgttaaaccaactagaa

SEQ ID NO: 617 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctggaaggatttcagcgattcgaaggctttcatcagcttttccaatcactcgcggttctatctagttacgcgttaaaccaactagaa

SEQ ID NO: 618 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctggaaggatttcagcgactcaaaggctttcatcagcttttccaatcactcgcggttctatctagttacgcgttaaaccaactagaa

SEQ ID NO: 619 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctgaaaggacttcagcgattcgaaggctttcatcagcttttccaatcactcgcggttctatctagttacgcgttaaaccaactagaa

SEQ ID NO: 620 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctgaaaggacttcagcgactcaaaggctttcatcagcttttccaatcactcgcggttctatctagttacgcgttaaaccaactagaa

SEQ ID NO: 621 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctggaaggacttcagcgattcaaaggctttcatcagcttttccaatcactcgcggttctatctagttacgcgttaaaccaactagaa

SEQ ID NO: 622 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctggaaggatttcagggattcgaacgctttcatcagcttttccaatcactcgcggttctatctagttacgcgttaaaccaactagaa

SEQ ID NO: 623 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctggaaggatttcagggactcaaacgctttcatcagcttttccaatcactcgcggttctatctagttacgcgttaaaccaactagaa

SEQ ID NO: 624 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctgaaaggacttcagggattcgaacgctttcatcagcttttccaatcactcgcggttctatctagttacgcgttaaaccaactagaa

SEQ ID NO: 625 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctgaaaggacttcagggactcaaacgctttcatcagcttttccaatcactcgcggttctatctagttacgcgttaaaccaactagaa

SEQ ID NO: 626 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctgaaaggatttcagggactcaaaggctttcatcagcttttccaatcactcgcggttctatctagttacgcgttaaaccaactagaa

SEQ ID NO: 627 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctggaaggatttcagggattcaaaggctttcatcagcttttccaatcactcgcggttctatctagttacgcgttaaaccaactagaa

SEQ ID NO: 628 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctgaaaggacttgagcgattcgaacgctttcatcagcttttccaatcactcgcggttctatctagttacgcgttaaaccaactagaa

SEQ ID NO: 629 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctgaaaggacttgagcgactcaaacgctttcatcagcttttccaatcactcgcggttctatctagttacgcgttaaaccaactagaa

SEQ ID NO: 630 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctggaaggacttgagcgattcaaacgctttcatcagcttttccaatcactcgcggttctatctagttacgcgttaaaccaactagaa

SEQ ID NO: 631 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctgaaaggatttgagcgactcaaaggctttcatcagcttttccaatcactcgcggttctatctagttacgcgttaaaccaactagaa

SEQ ID NO: 632 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctggaaggatttgagcgattcaaaggctttcatcagcttttccaatcactcgcggttctatctagttacgcgttaaaccaactagaa

SEQ ID NO: 633 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctggaaggatttgagggattcaaacgctttcatcagcttttccaatcactcgcggttctatctagttacgcgttaaaccaactagaa

SEQ ID NO: 634 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctgaaaggacttcagcgattcgaacgctttcatcagcttttccaatcactcgcggttctatctagttacgcgttaaaccaactagaa

SEQ ID NO: 635 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctgaaaggatttcagcgactcgaacgctttcatcagcttttccaatcactcgcggttctatctagttacgcgttaaaccaactagaa

SEQ ID NO: 636 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctggaaggatttcagcgattcgaacgctttcatcagcttttccaatcactcgcggttctatctagttacgcgttaaaccaactagaa

SEQ ID NO: 637 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctggaaggatttcagcgactcaaacgctttcatcagcttttccaatcactcgcggttctatctagttacgcgttaaaccaactagaa

SEQ ID NO: 638 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctgaaaggatttcagggattcgaacgctttcatcagcttttccaatcactcgcggttctatctagttacgcgttaaaccaactagaa

SEQ ID NO: 639 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctgaaaggatttcagggactcaaacgctttcatcagcttttccaatcactcgcggttctatctagttacgcgttaaaccaactagaa

SEQ ID NO: 640 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctgaaaggatttcagggattcaaaggctttcatcagcttttccaatcactcgcggttctatctagttacgcgttaaaccaactagaa

SEQ ID NO: 641 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctgaaaggatttcagcgattcgaaggctttcatcagcttttccaatcactcgcggttctatctagttacgcgttaaaccaactagaa

SEQ ID NO: 642 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctgaaaggacttcagcgattcaaaggctttcatcagcttttccaatcactcgcggttctatctagttacgcgttaaaccaactagaa

SEQ ID NO: 643 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctgaaaggacttcagggattcaaacgctttcatcagcttttccaatcactcgcggttctatctagttacgcgttaaaccaactagaa

SEQ ID NO: 644 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctgaaaggacttcagcgactcaaacgctttcatcagcttttccaatcactcgcggttctatctagttacgcgttaaaccaactagaa

SEQ ID NO: 645 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctggaaggacttcagcgattcaaacgctttcatcagcttttccaatcactcgcggttctatctagttacgcgttaaaccaactagaa

SEQ ID NO: 646 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctgaaaggatttgagcgactcaaacgctttcatcagcttttccaatcactcgcggttctatctagttacgcgttaaaccaactagaa

SEQ ID NO: 647 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctgaaaggacttgagtgactcaaacgctttcatcagcttttccaatcactcgcggttctatctagttacgcgttaaaccaactagaa

SEQ ID NO: 648 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctgaaaggatttgagggattcaaacgctttcatcagcttttccaatcactcgcggttctatctagttacgcgttaaaccaactagaa

SEQ ID NO: 649 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctgaaaggatttgagcgattcgaacgctttcatcagcttttccaatcactcgcggttctatctagttacgcgttaaaccaactagaa

SEQ ID NO: 650 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctgaaaggatttgagcgattcaaaggctttcatcagcttttccaatcactcgcggttctatctagttacgcgttaaaccaactagaa

SEQ ID NO: 651 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctggaaggatttcagggattcaaacgctttcatcagcttttccaatcactcgcggttctatctagttacgcgttaaaccaactagaa

SEQ ID NO: 652 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctggaaggatttgagcgattcaaacgctttcatcagcttttccaatcactcgcggttctatctagttacgcgttaaaccaactagaa

SEQ ID NO: 653 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctgaaaggatttcagcgattcgaacgctttcatcagcttttccaatcactcgcggttctatctagttacgcgttaaaccaactagaa

SEQ ID NO: 654 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctgaaaggatttcagcgactcaaacgctttcatcagcttttccaatcactcgcggttctatctagttacgcgttaaaccaactagaa

SEQ ID NO: 655 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctgaaaggatttgagtgactcaaacgctttcatcagcttttccaatcactcgcggttctatctagttacgcgttaaaccaactagaa

SEQ ID NO: 656 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctgaaaggatttcagggattcaaacgctttcatcagcttttccaatcactcgcggttctatctagttacgcgttaaaccaactagaa

SEQ ID NO: 657 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctgaaaggatttcagcgattcaaaggctttcatcagcttttccaatcactcgcggttctatctagttacgcgttaaaccaactagaa

SEQ ID NO: 658 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctggaaggatttcagcgattcaaacgctttcatcagcttttccaatcactcgcggttctatctagttacgcgttaaaccaactagaa

SEQ ID NO: 659 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctgaaaggatttcagcgattcaaacgctttcatcagcttttccaatcactcgcggttctatctagttacgcgttaaaccaactagaa

SEQ ID NO: 660 - DNA, 223 bp, synthetic construct
  source 1..223: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctgaaacgatttcagcgattcaaacgctttcatcagcttttccaatcactcgcggttctatctagttacgcgttaaaccaactagaa

SEQ ID NO: 661 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctggaaggacttgagcgactcgaaggctttcatcagcttttccaatcactcgcggttctatctagttacgcgttaaaccaactagaa

SEQ ID NO: 662 - DNA, 225 bp, synthetic construct
  source 1..225: mol_type = other DNA
gaccctggaaaagctgatgagtttaagagctagaaatagcaagtttaaataaggctagtccgttatcaacttgaaaaagtggcaccgagtcggtgccggcggcggcggcggcggcggtggcggctgttgctgctgctgctggaaggacttgagggactcgaaggctttcatcagcttttccaatcactcgcggttctatctagttacgcgttaaaccaactagaa

SEQ ID NO: 663 - DNA, 225 bp, synthetic construct
  source 1..225: mol_type = other DNA
gaccctggaaaagctgatgagtttaagagctagaaatagcaagtttaaataaggctagtccgttatcaacttgaaaaagtggcaccgagtcggtgccggcggcggcggcggcggcggtggcggctgttgctgctgctgctggaaggacttcagggactcgaaggctttcatcagcttttccaatcactcgcggttctatctagttacgcgttaaaccaactagaa

SEQ ID NO: 664 - DNA, 225 bp, synthetic construct
  source 1..225: mol_type = other DNA
gaccctggaaaagctgatgagtttaagagctagaaatagcaagtttaaataaggctagtccgttatcaacttgaaaaagtggcaccgagtcggtgccggcggcggcggcggcggcggtggcggctgttgctgctgctgctggaaggacttgagggattcgaaggctttcatcagcttttccaatcactcgcggttctatctagttacgcgttaaaccaactagaa

SEQ ID NO: 665 - DNA, 225 bp, synthetic construct
  source 1..225: mol_type = other DNA
gaccctggaaaagctgatgagtttaagagctagaaatagcaagtttaaataaggctagtccgttatcaacttgaaaaagtggcaccgagtcggtgccggcggcggcggcggcggcggtggcggctgttgctgctgctgctggaaggacttcagggattcgaaggctttcatcagcttttccaatcactcgcggttctatctagttacgcgttaaaccaactagaa

SEQ ID NO: 666 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctggaaggacttgagggactcgaaggcgttcatcagcttttccaatcactcgcggttctatctagttacgcgttaaaccaactagaa

SEQ ID NO: 667 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctggaaggacttgagggactcgaagggcttcatcagcttttccaatcactcgcggttctatctagttacgcgttaaaccaactagaa

SEQ ID NO: 668 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctggaaggacttgagggactcgaaggggttcatcagcttttccaatcactcgcggttctatctagttacgcgttaaaccaactagaa

SEQ ID NO: 669 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctggaacgacttcagcgactcgaaggctttcatcagcttttccaatcactcgcggttctatctagttacgcgttaaaccaactagaa

SEQ ID NO: 670 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctggaacgacttgagggactcgaaggctttcatcagcttttccaatcactcgcggttctatctagttacgcgttaaaccaactagaa

SEQ ID NO: 671 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctggaaggatttgagggactcgaaggctttcatcagcttttccaatcactcgcggttctatctagttacgcgttaaaccaactagaa

SEQ ID NO: 672 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctgaaaggacttgagggactcgaaggctttcatcagcttttccaatcactcgcggttctatctagttacgcgttaaaccaactagaa

SEQ ID NO: 673 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctggaaggacttgagggattcgaaggctttcatcagcttttccaatcactcgcggttctatctagttacgcgttaaaccaactagaa

SEQ ID NO: 674 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctggaaggacttgagggactcaaaggctttcatcagcttttccaatcactcgcggttctatctagttacgcgttaaaccaactagaa

SEQ ID NO: 675 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctggaaggacttgagggactcgaacgctttcatcagcttttccaatcactcgcggttctatctagttacgcgttaaaccaactagaa

SEQ ID NO: 676 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctggaaggatttcagggactcgaaggctttcatcagcttttccaatcactcgcggttctatctagttacgcgttaaaccaactagaa

SEQ ID NO: 677 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctgaaaggacttcagggactcgaaggctttcatcagcttttccaatcactcgcggttctatctagttacgcgttaaaccaactagaa

SEQ ID NO: 678 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctggaaggacttcagggattcgaaggctttcatcagcttttccaatcactcgcggttctatctagttacgcgttaaaccaactagaa

SEQ ID NO: 679 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctggaaggacttcagggactcaaaggctttcatcagcttttccaatcactcgcggttctatctagttacgcgttaaaccaactagaa

SEQ ID NO: 680 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctggaaggacttcagggactcgaacgctttcatcagcttttccaatcactcgcggttctatctagttacgcgttaaaccaactagaa

SEQ ID NO: 681 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctggaaggacttcagcgactcgaaggctttcatcagcttttccaatcactcgcggttctatctagttacgcgttaaaccaactagaa

SEQ ID NO: 682 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctggaacgacttcagggactcgaaggctttcatcagcttttccaatcactcgcggttctatctagttacgcgttaaaccaactagaa

SEQ ID NO: 683 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctgaaaggatttgagggactcgaaggctttcatcagcttttccaatcactcgcggttctatctagttacgcgttaaaccaactagaa

SEQ ID NO: 684 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctggaaggatttgagggattcgaaggctttcatcagcttttccaatcactcgcggttctatctagttacgcgttaaaccaactagaa

SEQ ID NO: 685 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctggaaggatttgagggactcaaaggctttcatcagcttttccaatcactcgcggttctatctagttacgcgttaaaccaactagaa

SEQ ID NO: 686 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctggaaggatttgagggactcgaacgctttcatcagcttttccaatcactcgcggttctatctagttacgcgttaaaccaactagaa

SEQ ID NO: 687 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctgaaaggacttgagggattcgaaggctttcatcagcttttccaatcactcgcggttctatctagttacgcgttaaaccaactagaa

SEQ ID NO: 688 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctgaaaggacttgagggactcaaaggctttcatcagcttttccaatcactcgcggttctatctagttacgcgttaaaccaactagaa

SEQ ID NO: 689 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctgaaaggacttgagggactcgaacgctttcatcagcttttccaatcactcgcggttctatctagttacgcgttaaaccaactagaa

SEQ ID NO: 690 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctggaaggacttgagggattcaaaggctttcatcagcttttccaatcactcgcggttctatctagttacgcgttaaaccaactagaa

SEQ ID NO: 691 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctggaaggacttgagggattcgaacgctttcatcagcttttccaatcactcgcggttctatctagttacgcgttaaaccaactagaa

SEQ ID NO: 692 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctggaaggacttgagggactcaaacgctttcatcagcttttccaatcactcgcggttctatctagttacgcgttaaaccaactagaa

SEQ ID NO: 693 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctggaaggatttgagcgactcgaaggctttcatcagcttttccaatcactcgcggttctatctagttacgcgttaaaccaactagaa

SEQ ID NO: 694 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctgaaaggacttgagcgactcgaaggctttcatcagcttttccaatcactcgcggttctatctagttacgcgttaaaccaactagaa

SEQ ID NO: 695 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctggaaggacttgagcgattcgaaggctttcatcagcttttccaatcactcgcggttctatctagttacgcgttaaaccaactagaa

SEQ ID NO: 696 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctggaaggacttgagcgactcaaaggctttcatcagcttttccaatcactcgcggttctatctagttacgcgttaaaccaactagaa

SEQ ID NO: 697 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctggaaggacttgagcgactcgaacgctttcatcagcttttccaatcactcgcggttctatctagttacgcgttaaaccaactagaa

SEQ ID NO: 698 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctggaaggacttcagcgattcgaaggctttcatcagcttttccaatcactcgcggttctatctagttacgcgttaaaccaactagaa

SEQ ID NO: 699 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctggaaggacttcagcgactcgaacgctttcatcagcttttccaatcactcgcggttctatctagttacgcgttaaaccaactagaa

SEQ ID NO: 700 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctggaaggatttcagcgactcgaaggctttcatcagcttttccaatcactcgcggttctatctagttacgcgttaaaccaactagaa

SEQ ID NO: 701 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctgaaaggacttcagcgactcgaaggctttcatcagcttttccaatcactcgcggttctatctagttacgcgttaaaccaactagaa

SEQ ID NO: 702 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctggaaggacttcagcgactcaaaggctttcatcagcttttccaatcactcgcggttctatctagttacgcgttaaaccaactagaa

SEQ ID NO: 703 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctgaaaggatttgagggattcgaaggctttcatcagcttttccaatcactcgcggttctatctagttacgcgttaaaccaactagaa

SEQ ID NO: 704 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctgaaaggatttgagggactcaaaggctttcatcagcttttccaatcactcgcggttctatctagttacgcgttaaaccaactagaa

SEQ ID NO: 705 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctgaaaggatttgagggactcgaacgctttcatcagcttttccaatcactcgcggttctatctagttacgcgttaaaccaactagaa

SEQ ID NO: 706 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctgaaaggatttcagggactcgaaggctttcatcagcttttccaatcactcgcggttctatctagttacgcgttaaaccaactagaa

SEQ ID NO: 707 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctgaaaggacttgagggattcaaaggctttcatcagcttttccaatcactcgcggttctatctagttacgcgttaaaccaactagaa

SEQ ID NO: 708 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctgaaaggacttgagggattcgaacgctttcatcagcttttccaatcactcgcggttctatctagttacgcgttaaaccaactagaa

SEQ ID NO: 709 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctgaaaggacttcagggattcgaaggctttcatcagcttttccaatcactcgcggttctatctagttacgcgttaaaccaactagaa

SEQ ID NO: 710 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctggaaggacttcagggactcaaacgctttcatcagcttttccaatcactcgcggttctatctagttacgcgttaaaccaactagaa

SEQ ID NO: 711 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctggaaggatttgagggattcgaacgctttcatcagcttttccaatcactcgcggttctatctagttacgcgttaaaccaactagaa

SEQ ID NO: 712 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctggaaggatttcagggattcgaaggctttcatcagcttttccaatcactcgcggttctatctagttacgcgttaaaccaactagaa

SEQ ID NO: 713 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctggaaggatttgagggactcaaacgctttcatcagcttttccaatcactcgcggttctatctagttacgcgttaaaccaactagaa

SEQ ID NO: 714 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctggaaggatttgagcgattcgaaggctttcatcagcttttccaatcactcgcggttctatctagttacgcgttaaaccaactagaa

SEQ ID NO: 715 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctgaaaggacttgagcgattcgaaggctttcatcagcttttccaatcactcgcggttctatctagttacgcgttaaaccaactagaa

SEQ ID NO: 716 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctggaaggacttgagcgattcaaaggctttcatcagcttttccaatcactcgcggttctatctagttacgcgttaaaccaactagaa

SEQ ID NO: 717 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctggaaggatttgagcgactcgaacgctttcatcagcttttccaatcactcgcggttctatctagttacgcgttaaaccaactagaa

SEQ ID NO: 718 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctgaaaggacttgagcgactcgaacgctttcatcagcttttccaatcactcgcggttctatctagttacgcgttaaaccaactagaa

SEQ ID NO: 719 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctggaaggacttgagcgattcgaacgctttcatcagcttttccaatcactcgcggttctatctagttacgcgttaaaccaactagaa

SEQ ID NO: 720 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctggaaggacttgagcgactcaaacgctttcatcagcttttccaatcactcgcggttctatctagttacgcgttaaaccaactagaa

SEQ ID NO: 721 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctgaaaggatttgagcgactcgaaggctttcatcagcttttccaatcactcgcggttctatctagttacgcgttaaaccaactagaa

SEQ ID NO: 722 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctggaaggatttgagcgactcaaaggctttcatcagcttttccaatcactcgcggttctatctagttacgcgttaaaccaactagaa

SEQ ID NO: 723 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctggaaggatttcagggactcgaacgctttcatcagcttttccaatcactcgcggttctatctagttacgcgttaaaccaactagaa

SEQ ID NO: 724 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctgaaaggacttcagggactcgaacgctttcatcagcttttccaatcactcgcggttctatctagttacgcgttaaaccaactagaa

SEQ ID NO: 725 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctggaaggacttcagggattcgaacgctttcatcagcttttccaatcactcgcggttctatctagttacgcgttaaaccaactagaa

SEQ ID NO: 726 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctgaaaggacttcagggactcaaaggctttcatcagcttttccaatcactcgcggttctatctagttacgcgttaaaccaactagaa

SEQ ID NO: 727 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctgaaaggacttgagcgactcaaaggctttcatcagcttttccaatcactcgcggttctatctagttacgcgttaaaccaactagaa

SEQ ID NO: 728 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctgaaaggacttgagggactcaaacgctttcatcagcttttccaatcactcgcggttctatctagttacgcgttaaaccaactagaa

SEQ ID NO: 729 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctggaaggacttcagcgattcgaacgctttcatcagcttttccaatcactcgcggttctatctagttacgcgttaaaccaactagaa

SEQ ID NO: 730 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctggaaggatttcagcgactcgaacgctttcatcagcttttccaatcactcgcggttctatctagttacgcgttaaaccaactagaa

SEQ ID NO: 731 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctgaaaggacttcagcgactcgaacgctttcatcagcttttccaatcactcgcggttctatctagttacgcgttaaaccaactagaa

SEQ ID NO: 732 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctggaaggacttcagcgactcaaacgctttcatcagcttttccaatcactcgcggttctatctagttacgcgttaaaccaactagaa

SEQ ID NO: 733 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctgaaaggatttgagcgactcgaacgctttcatcagcttttccaatcactcgcggttctatctagttacgcgttaaaccaactagaa

SEQ ID NO: 734 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctggaaggatttgagcgattcgaacgctttcatcagcttttccaatcactcgcggttctatctagttacgcgttaaaccaactagaa

SEQ ID NO: 735 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctgaaaggatttgagggattcgaacgctttcatcagcttttccaatcactcgcggttctatctagttacgcgttaaaccaactagaa

SEQ ID NO: 736 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctgaaaggatttgagggactcaaacgctttcatcagcttttccaatcactcgcggttctatctagttacgcgttaaaccaactagaa

SEQ ID NO: 737 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctgaaaggatttcagggactcgaacgctttcatcagcttttccaatcactcgcggttctatctagttacgcgttaaaccaactagaa

SEQ ID NO: 738 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctgaaaggatttgagggattcaaaggctttcatcagcttttccaatcactcgcggttctatctagttacgcgttaaaccaactagaa

SEQ ID NO: 739 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctgaaaggatttcagggattcgaaggctttcatcagcttttccaatcactcgcggttctatctagttacgcgttaaaccaactagaa

SEQ ID NO: 740 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctgaaaggatttgagcgattcgaaggctttcatcagcttttccaatcactcgcggttctatctagttacgcgttaaaccaactagaa

SEQ ID NO: 741 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctgaaaggacttcagggattcaaaggctttcatcagcttttccaatcactcgcggttctatctagttacgcgttaaaccaactagaa

SEQ ID NO: 742 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctgaaaggacttgagcgattcaaaggctttcatcagcttttccaatcactcgcggttctatctagttacgcgttaaaccaactagaa

SEQ ID NO: 743 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctgaaaggacttgagggattcaaacgctttcatcagcttttccaatcactcgcggttctatctagttacgcgttaaaccaactagaa

SEQ ID NO: 744 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctgaaaggatttcagcgactcgaaggctttcatcagcttttccaatcactcgcggttctatctagttacgcgttaaaccaactagaa

SEQ ID NO: 745 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctggaaggatttcagcgattcgaaggctttcatcagcttttccaatcactcgcggttctatctagttacgcgttaaaccaactagaa

SEQ ID NO: 746 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctggaaggatttcagcgactcaaaggctttcatcagcttttccaatcactcgcggttctatctagttacgcgttaaaccaactagaa

SEQ ID NO: 747 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctgaaaggacttcagcgattcgaaggctttcatcagcttttccaatcactcgcggttctatctagttacgcgttaaaccaactagaa

SEQ ID NO: 748 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctgaaaggacttcagcgactcaaaggctttcatcagcttttccaatcactcgcggttctatctagttacgcgttaaaccaactagaa

SEQ ID NO: 749 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctggaaggacttcagcgattcaaaggctttcatcagcttttccaatcactcgcggttctatctagttacgcgttaaaccaactagaa

SEQ ID NO: 750 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctggaaggatttcagggattcgaacgctttcatcagcttttccaatcactcgcggttctatctagttacgcgttaaaccaactagaa

SEQ ID NO: 751 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctggaaggatttcagggactcaaacgctttcatcagcttttccaatcactcgcggttctatctagttacgcgttaaaccaactagaa

SEQ ID NO: 752 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctgaaaggacttcagggattcgaacgctttcatcagcttttccaatcactcgcggttctatctagttacgcgttaaaccaactagaa

SEQ ID NO: 753 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctgaaaggacttcagggactcaaacgctttcatcagcttttccaatcactcgcggttctatctagttacgcgttaaaccaactagaa

SEQ ID NO: 754 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctgaaaggatttcagggactcaaaggctttcatcagcttttccaatcactcgcggttctatctagttacgcgttaaaccaactagaa

SEQ ID NO: 755 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctggaaggatttcagggattcaaaggctttcatcagcttttccaatcactcgcggttctatctagttacgcgttaaaccaactagaa

SEQ ID NO: 756 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctgaaaggacttgagcgattcgaacgctttcatcagcttttccaatcactcgcggttctatctagttacgcgttaaaccaactagaa

SEQ ID NO: 757 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctgaaaggacttgagcgactcaaacgctttcatcagcttttccaatcactcgcggttctatctagttacgcgttaaaccaactagaa

SEQ ID NO: 758 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctggaaggacttgagcgattcaaacgctttcatcagcttttccaatcactcgcggttctatctagttacgcgttaaaccaactagaa

SEQ ID NO: 759 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctgaaaggatttgagcgactcaaaggctttcatcagcttttccaatcactcgcggttctatctagttacgcgttaaaccaactagaa

SEQ ID NO: 760 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctggaaggatttgagcgattcaaaggctttcatcagcttttccaatcactcgcggttctatctagttacgcgttaaaccaactagaa

SEQ ID NO: 761 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctggaaggatttgagggattcaaacgctttcatcagcttttccaatcactcgcggttctatctagttacgcgttaaaccaactagaa

SEQ ID NO: 762 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctgaaaggacttcagcgattcgaacgctttcatcagcttttccaatcactcgcggttctatctagttacgcgttaaaccaactagaa

SEQ ID NO: 763 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctgaaaggatttcagcgactcgaacgctttcatcagcttttccaatcactcgcggttctatctagttacgcgttaaaccaactagaa

SEQ ID NO: 764 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctggaaggatttcagcgattcgaacgctttcatcagcttttccaatcactcgcggttctatctagttacgcgttaaaccaactagaa

SEQ ID NO: 765 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctggaaggatttcagcgactcaaacgctttcatcagcttttccaatcactcgcggttctatctagttacgcgttaaaccaactagaa

SEQ ID NO: 766 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctgaaaggatttcagggattcgaacgctttcatcagcttttccaatcactcgcggttctatctagttacgcgttaaaccaactagaa

SEQ ID NO: 767 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctgaaaggatttcagggactcaaacgctttcatcagcttttccaatcactcgcggttctatctagttacgcgttaaaccaactagaa

SEQ ID NO: 768 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctgaaaggatttcagggattcaaaggctttcatcagcttttccaatcactcgcggttctatctagttacgcgttaaaccaactagaa

SEQ ID NO: 769 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctgaaaggatttcagcgattcgaaggctttcatcagcttttccaatcactcgcggttctatctagttacgcgttaaaccaactagaa

SEQ ID NO: 770 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctgaaaggacttcagcgattcaaaggctttcatcagcttttccaatcactcgcggttctatctagttacgcgttaaaccaactagaa

SEQ ID NO: 771 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctgaaaggacttcagggattcaaacgctttcatcagcttttccaatcactcgcggttctatctagttacgcgttaaaccaactagaa

SEQ ID NO: 772 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctgaaaggacttcagcgactcaaacgctttcatcagcttttccaatcactcgcggttctatctagttacgcgttaaaccaactagaa

SEQ ID NO: 773 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctggaaggacttcagcgattcaaacgctttcatcagcttttccaatcactcgcggttctatctagttacgcgttaaaccaactagaa

SEQ ID NO: 774 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctgaaaggatttgagcgactcaaacgctttcatcagcttttccaatcactcgcggttctatctagttacgcgttaaaccaactagaa

SEQ ID NO: 775 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctgaaaggacttgagtgactcaaacgctttcatcagcttttccaatcactcgcggttctatctagttacgcgttaaaccaactagaa

SEQ ID NO: 776 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctgaaaggatttgagggattcaaacgctttcatcagcttttccaatcactcgcggttctatctagttacgcgttaaaccaactagaa

SEQ ID NO: 777 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctgaaaggatttgagcgattcgaacgctttcatcagcttttccaatcactcgcggttctatctagttacgcgttaaaccaactagaa

SEQ ID NO: 778 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctgaaaggatttgagcgattcaaaggctttcatcagcttttccaatcactcgcggttctatctagttacgcgttaaaccaactagaa

SEQ ID NO: 779 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctggaaggatttcagggattcaaacgctttcatcagcttttccaatcactcgcggttctatctagttacgcgttaaaccaactagaa

SEQ ID NO: 780 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctggaaggatttgagcgattcaaacgctttcatcagcttttccaatcactcgcggttctatctagttacgcgttaaaccaactagaa

SEQ ID NO: 781 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctgaaaggatttcagcgattcgaacgctttcatcagcttttccaatcactcgcggttctatctagttacgcgttaaaccaactagaa

SEQ ID NO: 782 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctgaaaggatttcagcgactcaaacgctttcatcagcttttccaatcactcgcggttctatctagttacgcgttaaaccaactagaa

SEQ ID NO: 783 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctgaaaggatttgagtgactcaaacgctttcatcagcttttccaatcactcgcggttctatctagttacgcgttaaaccaactagaa

SEQ ID NO: 784 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctgaaaggatttcagggattcaaacgctttcatcagcttttccaatcactcgcggttctatctagttacgcgttaaaccaactagaa

SEQ ID NO: 785 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctgaaaggatttcagcgattcaaaggctttcatcagcttttccaatcactcgcggttctatctagttacgcgttaaaccaactagaa

SEQ ID NO: 786 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctggaaggatttcagcgattcaaacgctttcatcagcttttccaatcactcgcggttctatctagttacgcgttaaaccaactagaa

SEQ ID NO: 787 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctgaaaggatttcagcgattcaaacgctttcatcagcttttccaatcactcgcggttctatctagttacgcgttaaaccaactagaa

SEQ ID NO: 788 - DNA, 232 bp, synthetic construct
  source 1..232: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctgaaacgatttcagcgattcaaacgctttcatcagcttttccaatcactcgcggttctatctagttacgcgttaaaccaactagaa

SEQ ID NO: 789 - DNA, 238 bp, synthetic construct
  source 1..238: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctgctgctgctgctgctggaaggacttcagggactcgaaggctttcatcagcttttccaatcactcgcggttctatctagttacgcgttaaaccaactagaa

SEQ ID NO: 790 - DNA, 238 bp, synthetic construct
  source 1..238: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgctgctgctgctgctgctgctggaaggacttgagggattcgaaggctttcatcagcttttccaatcactcgcggttctatctagttacgcgttaaaccaactagaa

SEQ ID NO: 791 - DNA, 238 bp, synthetic construct
  source 1..238: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgttgttgttgttgttgttgttgttgttggaaggacttgagggactcgaaggctttcatcagcttttccaatcactcgcggttctatctagttacgcgttaaaccaactagaa

SEQ ID NO: 792 - DNA, 238 bp, synthetic construct
  source 1..238: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgttgctgttgctgttgctgttggaaggacttgagggactcgaaggctttcatcagcttttccaatcactcgcggttctatctagttacgcgttaaaccaactagaa

SEQ ID NO: 793 - DNA, 238 bp, synthetic construct
  source 1..238: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgttgctgttgctgttgctgttgctgttggaaggacttcagggactcgaaggctttcatcagcttttccaatcactcgcggttctatctagttacgcgttaaaccaactagaa

SEQ ID NO: 794 - DNA, 238 bp, synthetic construct
  source 1..238: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgttgctgttgctgttgctgttgctgttggaaggacttgagggattcgaaggctttcatcagcttttccaatcactcgcggttctatctagttacgcgttaaaccaactagaa

SEQ ID NO: 795 - DNA, 238 bp, synthetic construct
  source 1..238: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgttgttgttgttgttgttgttgttgttggaaggacttcagggactcgaaggctttcatcagcttttccaatcactcgcggttctatctagttacgcgttaaaccaactagaa

SEQ ID NO: 796 - DNA, 238 bp, synthetic construct
  source 1..238: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgttgttgttgttgttgttgttgttgttggaaggacttgagggattcgaaggctttcatcagcttttccaatcactcgcggttctatctagttacgcgttaaaccaactagaa

SEQ ID NO: 797 - DNA, 238 bp, synthetic construct
  source 1..238: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgttgttgttgttgttgttgttgttgttggaaggacttcagggattcgaaggctttcatcagcttttccaatcactcgcggttctatctagttacgcgttaaaccaactagaa

SEQ ID NO: 798 - DNA, 238 bp, synthetic construct
  source 1..238: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgttgctgttgctgttgctgttggaaggacttcagggactcgaaggctttcatcagcttttccaatcactcgcggttctatctagttacgcgttaaaccaactagaa

SEQ ID NO: 799 - DNA, 238 bp, synthetic construct
  source 1..238: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgttgctgttgctgttgctgttggaaggacttgagggattcgaaggctttcatcagcttttccaatcactcgcggttctatctagttacgcgttaaaccaactagaa

SEQ ID NO: 800 - DNA, 238 bp, synthetic construct
  source 1..238: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgctgttgctgttgctgttgctgttggaaggacttcagggattcgaaggctttcatcagcttttccaatcactcgcggttctatctagttacgcgttaaaccaactagaa

SEQ ID NO: 801 - DNA, 247 bp, synthetic construct
  source 1..247: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgttgttgttgttgttgttgttgttgttggaaggacttgagggactcgaaggctttcatcagcttttccaatcactcgcggttctatctagttacgcgttaaaccaactagaa

SEQ ID NO: 802 - DNA, 247 bp, synthetic construct
  source 1..247: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgttgctgttgctgttgctgttggaaggacttgagggactcgaaggctttcatcagcttttccaatcactcgcggttctatctagttacgcgttaaaccaactagaa

SEQ ID NO: 803 - DNA, 247 bp, synthetic construct
  source 1..247: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgttgctgttgctgttgctgttgctgttggaaggacttcagggactcgaaggctttcatcagcttttccaatcactcgcggttctatctagttacgcgttaaaccaactagaa

SEQ ID NO: 804 - DNA, 247 bp, synthetic construct
  source 1..247: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgttgctgttgctgttgctgttgctgttggaaggacttgagggattcgaaggctttcatcagcttttccaatcactcgcggttctatctagttacgcgttaaaccaactagaa

SEQ ID NO: 805 - DNA, 247 bp, synthetic construct
  source 1..247: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgttgttgttgttgttgttgttgttgttggaaggacttcagggactcgaaggctttcatcagcttttccaatcactcgcggttctatctagttacgcgttaaaccaactagaa

SEQ ID NO: 806 - DNA, 247 bp, synthetic construct
  source 1..247: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgttgttgttgttgttgttgttgttgttggaaggacttgagggattcgaaggctttcatcagcttttccaatcactcgcggttctatctagttacgcgttaaaccaactagaa

SEQ ID NO: 807 - DNA, 247 bp, synthetic construct
  source 1..247: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgttgttgttgttgttgttgttgttgttggaaggacttcagggattcgaaggctttcatcagcttttccaatcactcgcggttctatctagttacgcgttaaaccaactagaa

SEQ ID NO: 808 - DNA, 247 bp, synthetic construct
  source 1..247: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgttgctgttgctgttgctgttggaaggacttcagggactcgaaggctttcatcagcttttccaatcactcgcggttctatctagttacgcgttaaaccaactagaa

SEQ ID NO: 809 - DNA, 247 bp, synthetic construct
  source 1..247: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgttgctgttgctgttgctgttggaaggacttgagggattcgaaggctttcatcagcttttccaatcactcgcggttctatctagttacgcgttaaaccaactagaa

SEQ ID NO: 810 - DNA, 247 bp, synthetic construct
  source 1..247: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgttgctgttgctgttgctgttggaaggacttcagggattcgaaggctttcatcagcttttccaatcactcgcggttctatctagttacgcgttaaaccaactagaa

SEQ ID NO: 811 - DNA, 247 bp, synthetic construct
  source 1..247: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctgctgctgctgctgctggaaggacttcagggactcgaaggctttcatcagcttttccaatcactcgcggttctatctagttacgcgttaaaccaactagaa

SEQ ID NO: 812 - DNA, 247 bp, synthetic construct
  source 1..247: mol_type = other DNA
gaccctggaaaagctgatgagtttaagagctagaaatagcaagtttaaataaggctagtccgttatcaacttgaaaaagtggcaccgagtcggtgcgaggaggcggcggcggcggcggcggcggtggcggctgttgctgctgctgctgctgctgctgctgctggaaggacttgagggattcgaaggctttcatcagcttttccaatcactcgcggttctatctagttacgcgttaaaccaactagaa

SEQ ID NO: 813 - DNA, 238 bp, synthetic construct
  source 1..238: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgttgctgctgttgctgctgttgctgctggaaggacttgagggactcgaaggctttcatcagcttttccaatcactcgcggttctatctagttacgcgttaaaccaactagaa

SEQ ID NO: 814 - DNA, 238 bp, synthetic construct
  source 1..238: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgctgttgctgctgctgttgctgctgctggaaggacttgagggactcgaaggctttcatcagcttttccaatcactcgcggttctatctagttacgcgttaaaccaactagaa

SEQ ID NO: 815 - DNA, 238 bp, synthetic construct
  source 1..238: mol_type = other DNA
gaccctggaaaagctgatgagtttaagagctagaaatagcaagtttaaataaggctagtccgttatcaacttgaaaaagtggcaccgagtcggtgcgcggcggcggcggcggcggtggcggctgttgttgttgctgttgttgctgttgttgctggaaggacttgagggactcgaaggctttcatcagcttttccaatcactcgcggttctatctagttacgcgttaaaccaactagaa

SEQ ID NO: 816 - DNA, 137 bp, synthetic construct
  source 1..137: mol_type = other DNA
gcaagactaacctggccaacagttttagagctagaaatagcaagttaaaataaggctagtccgttatcaacttgaaaaagtggcaccgagtcggtgcattacaggcgcgcgacaccacgcccggctaactggccagg

SEQ ID NO: 817 - DNA, 139 bp, synthetic construct
  source 1..139: mol_type = other DNA
gcaagactaacctggccaacagttttagagctagaaatagcaagttaaaataaggctagtccgttatcaacttgaaaaagtggcaccgagtcggtgcattacaggcgcgcgacaccacgcccggctaactggccaggtt

SEQ ID NO: 818 - DNA, 141 bp, synthetic construct
  source 1..141: mol_type = other DNA
gcaagactaacctggccaacagttttagagctagaaatagcaagttaaaataaggctagtccgttatcaacttgaaaaagtggcaccgagtcggtgcattacaggcgcgcgacaccacgcccggctaactggccaggttag

SEQ ID NO: 819 - DNA, 143 bp, synthetic construct
  source 1..143: mol_type = other DNA
gcaagactaacctggccaacagttttagagctagaaatagcaagttaaaataaggctagtccgttatcaacttgaaaaagtggcaccgagtcggtgcattacaggcgcgcgacaccacgcccggctaactggccaggttagtc

SEQ ID NO: 820 - DNA, 140 bp, synthetic construct
  source 1..140: mol_type = other DNA
gcaagactaacctggccaacagttttagagctagaaatagcaagttaaaataaggctagtccgttatcaacttgaaaaagtggcaccgagtcggtgcgggattacaggcgcgcgacaccacgcccggctaactggccagg

SEQ ID NO: 821 - DNA, 142 bp, synthetic construct
  source 1..142: mol_type = other DNA
gcaagactaacctggccaacagttttagagctagaaatagcaagttaaaataaggctagtccgttatcaacttgaaaaagtggcaccgagtcggtgcgggattacaggcgcgcgacaccacgcccggctaactggccaggtt

SEQ ID NO: 822 - DNA, 144 bp, synthetic construct
  source 1..144: mol_type = other DNA
gcaagactaacctggccaacagttttagagctagaaatagcaagttaaaataaggctagtccgttatcaacttgaaaaagtggcaccgagtcggtgcgggattacaggcgcgcgacaccacgcccggctaactggccaggttag

SEQ ID NO: 823 - DNA, 146 bp, synthetic construct
  source 1..146: mol_type = other DNA
gcaagactaacctggccaacagttttagagctagaaatagcaagttaaaataaggctagtccgttatcaacttgaaaaagtggcaccgagtcggtgcgggattacaggcgcgcgacaccacgcccggctaactggccaggttagtc

SEQ ID NO: 824 - DNA, 145 bp, synthetic construct
  source 1..145: mol_type = other DNA
gcaagactaacctggccaacagttttagagctagaaatagcaagttaaaataaggctagtccgttatcaacttgaaaaagtggcaccgagtcggtgctagctgggattacaggcgcgcgacaccacgcccggctaactggccagg

SEQ ID NO: 825 - DNA, 147 bp, synthetic construct
  source 1..147: mol_type = other DNA
gcaagactaacctggccaacagttttagagctagaaatagcaagttaaaataaggctagtccgttatcaacttgaaaaagtggcaccgagtcggtgctagctgggattacaggcgcgcgacaccacgcccggctaactggccaggtt

SEQ ID NO: 826 - DNA, 149 bp, synthetic construct
  source 1..149: mol_type = other DNA
gcaagactaacctggccaacagttttagagctagaaatagcaagttaaaataaggctagtccgttatcaacttgaaaaagtggcaccgagtcggtgctagctgggattacaggcgcgcgacaccacgcccggctaactggccaggttag

SEQ ID NO: 827 - DNA, 151 bp, synthetic construct
  source 1..151: mol_type = other DNA
gcaagactaacctggccaacagttttagagctagaaatagcaagttaaaataaggctagtccgttatcaacttgaaaaagtggcaccgagtcggtgctagctgggattacaggcgcgcgacaccacgcccggctaactggccaggttagtc

SEQ ID NO: 828 - DNA, 190 bp, synthetic construct
  source 1..190: mol_type = other DNA
gcaagactaacctggccaacagttttagagctagaaatagcaagttaaaataaggctagtccgttatcaacttgaaaaagtggcaccgagtcggtgctagctgggattacaggcgcgcgacaccacgcccggctaactggccaggcaatcactcgcggttctatctagttacgcgttaaaccaactagaa

SEQ ID NO: 829 - DNA, 192 bp, synthetic construct
  source 1..192: mol_type = other DNA
gcaagactaacctggccaacagttttagagctagaaatagcaagttaaaataaggctagtccgttatcaacttgaaaaagtggcaccgagtcggtgctagctgggattacaggcgcgcgacaccacgcccggctaactggccaggttcaatcactcgcggttctatctagttacgcgttaaaccaactagaa

SEQ ID NO: 830 - DNA, 194 bp, synthetic construct
  source 1..194: mol_type = other DNA
gcaagactaacctggccaacagttttagagctagaaatagcaagttaaaataaggctagtccgttatcaacttgaaaaagtggcaccgagtcggtgctagctgggattacaggcgcgcgacaccacgcccggctaactggccaggttagcaatcactcgcggttctatctagttacgcgttaaaccaactagaa

SEQ ID NO: 831 - DNA, 196 bp, synthetic construct
  source 1..196: mol_type = other DNA
gcaagactaacctggccaacagttttagagctagaaatagcaagttaaaataaggctagtccgttatcaacttgaaaaagtggcaccgagtcggtgctagctgggattacaggcgcgcgacaccacgcccggctaactggccaggttagtccaatcactcgcggttctatctagttacgcgttaaaccaactagaa

SEQ ID NO: 832 - DNA, 190 bp, synthetic construct
  source 1..190: mol_type = other DNA
gcaagactaacctggccaacagttttagagctagaaatagcaagttaaaataaggctagtccgttatcaacttgaaaaagtggcaccgagtcggtgctagctgggattacaggcgcgcgacaccacgcccggctaactggccaggcaaataaccgcggttctatctagttacgcgttaaaccaactagaa

SEQ ID NO: 833 - DNA, 192 bp, synthetic construct
  source 1..192: mol_type = other DNA
gcaagactaacctggccaacagttttagagctagaaatagcaagttaaaataaggctagtccgttatcaacttgaaaaagtggcaccgagtcggtgctagctgggattacaggcgcgcgacaccacgcccggctaactggccaggttcaaataaccgcggttctatctagttacgcgttaaaccaactagaa

SEQ ID NO: 834 - DNA, 194 bp, synthetic construct
  source 1..194: mol_type = other DNA
gcaagactaacctggccaacagttttagagctagaaatagcaagttaaaataaggctagtccgttatcaacttgaaaaagtggcaccgagtcggtgctagctgggattacaggcgcgcgacaccacgcccggctaactggccaggttagcaaataaccgcggttctatctagttacgcgttaaaccaactagaa

SEQ ID NO: 835 - DNA, 196 bp, synthetic construct
  source 1..196: mol_type = other DNA
gcaagactaacctggccaacagttttagagctagaaatagcaagttaaaataaggctagtccgttatcaacttgaaaaagtggcaccgagtcggtgctagctgggattacaggcgcgcgacaccacgcccggctaactggccaggttagtccaaataaccgcggttctatctagttacgcgttaaaccaactagaa

SEQ ID NO: 836 - DNA, 180 bp, synthetic construct
  source 1..180: mol_type = other DNA
gcaagactaacctggccaacagttttagagctagaaatagcaagttaaaataaggctagtccgttatcaacttgaaaaagtggcaccgagtcggtgctcctgccgcagcctctggagtagctgggattggccaggcaaataaccgcggttctatctagttacgcgttaaaccaactagaa

SEQ ID NO: 837 - DNA, 185 bp, synthetic construct
  source 1..185: mol_type = other DNA
gtctggagtagctgggattacgttttagagctagaaatagcaagttaaaataaggctagtccgttatcaacttgaaaaagtggcaccgagtcggtgctgaggtccggagttcaagactaacctggccaatcccagctactcaaataaccgcggttctatctagttacgcgttaaaccaactagaa

SEQ ID NO: 838 - DNA, 185 bp, synthetic construct
  source 1..185: mol_type = other DNA
gcaagactaacctggccaacagttttagagctagaaatagcaagttaaaataaggctagtccgttatcaacttgaaaaagtggcaccgagtcggtgcagtagctgggattacaggcgcgcgacaccactggccaggttagcaaataaccgcggttctatctagttacgcgttaaaccaactagaa

SEQ ID NO: 839 - DNA, 183 bp, synthetic construct
  source 1..183: mol_type = other DNA
gcaggcgcgcgacaccacgccgttttagagctagaaatagcaagttaaaataaggctagtccgttatcaacttgaaaaagtggcaccgagtcggtgctgaggtccggagttcaagactaacctggccagtggtgtcgccaaataaccgcggttctatctagttacgcgttaaaccaactagaa

SEQ ID NO: 840 - DNA, 188 bp, synthetic construct
  source 1..188: mol_type = other DNA
gcaagactaacctggccaacagttttagagctagaaatagcaagttaaaataaggctagtccgttatcaacttgaaaaagtggcaccgagtcggtgctacaggcgcgcgacaccacatactgggtttcaccatgttggccaggcaaataaccgcggttctatctagttacgcgttaaaccaactagaa

SEQ ID NO: 841 - DNA, 174 bp, synthetic construct
  source 1..174: mol_type = other DNA
gcaagactaacctggccaacagttttagagctagaaatagcaagttaaaataaggctagtccgttatcaacttgaaaaagtggcaccgagtcggtgctagatactgggtttcaccatgttggccaggttcaaataaccgcggttctatctagttacgcgttaaaccaactagaa

SEQ ID NO: 842 - DNA, 192 bp, synthetic construct
  source 1..192: mol_type = other DNA
gtctggagtagctgggattacgttttagagctagaaatagcaagttaaaataaggctagtccgttatcaacttgaaaaagtggcaccgagtcggtgcacatggtgaaacccagtatgtggtgtcgcgcgcctgtaatcccagctactcaaataaccgcggttctatctagttacgcgttaaaccaactagaa

SEQ ID NO: 843 - DNA, 176 bp, synthetic construct
  source 1..176: mol_type = other DNA
gcaggcgcgcgacaccacgccgttttagagctagaaatagcaagttaaaataaggctagtccgttatcaacttgaaaaagtggcaccgagtcggtgcacatggtgaaacccagtatctagtggtgtcgcgccaaataaccgcggttctatctagttacgcgttaaaccaactagaa

SEQ ID NO: 844 - DNA, 174 bp, synthetic construct
  source 1..174: mol_type = other DNA
gcaagactaacctggccaacagttttagagctagaaatagcaagttaaaataaggctagtccgttatcaacttgaaaaagtggcaccgagtcggtgctacaggcgcgcgacaccacgcctggccaggttcaaataaccgcggttctatctagttacgcgttaaaccaactagaa

SEQ ID NO: 845 - DNA, 174 bp, synthetic construct
  source 1..174: mol_type = other DNA
gcaagactaacctggccaacagttttagagctagaaatagcaagttaaaataaggctagtccgttatcaacttgaaaaagtggcaccgagtcggtgcgcccggctaacttttctttatggccaggttagcaaataaccgcggttctatctagttacgcgttaaaccaactagaa

SEQ ID NO: 846 - DNA, 174 bp, synthetic construct
  source 1..174: mol_type = other DNA
gcaagactaacctggccaacagttttagagctagaaatagcaagttaaaataaggctagtccgttatcaacttgaaaaagtggcaccgagtcggtgcacatggtgaaacccagtatctaatcccagctacaaataaccgcggttctatctagttacgcgttaaaccaactagaa

SEQ ID NO: 847 - DNA, 174 bp, synthetic construct
  source 1..174: mol_type = other DNA
gcaagactaacctggccaacagttttagagctagaaatagcaagttaaaataaggctagtccgttatcaacttgaaaaagtggcaccgagtcggtgcacatggtgaaacccagtatctagtggtgtcgccaaataaccgcggttctatctagttacgcgttaaaccaactagaa

SEQ ID NO: 848 - DNA, 135 bp, synthetic construct
  source 1..135: mol_type = other DNA
gcaagactaacctggccaacagttttagagctagaaatagcaagttaaaataaggctagtccgttatcaacttgaaaaagtggcaccgagtcggtgctcctgccgcagcctctggagtagctgggattggccagg

SEQ ID NO: 849 - DNA, 140 bp, synthetic construct
  source 1..140: mol_type = other DNA
gtctggagtagctgggattacgttttagagctagaaatagcaagttaaaataaggctagtccgttatcaacttgaaaaagtggcaccgagtcggtgctgaggtccggagttcaagactaacctggccaatcccagctact

SEQ ID NO: 850 - DNA, 140 bp, synthetic construct
  source 1..140: mol_type = other DNA
gcaagactaacctggccaacagttttagagctagaaatagcaagttaaaataaggctagtccgttatcaacttgaaaaagtggcaccgagtcggtgcagtagctgggattacaggcgcgcgacaccactggccaggttag

SEQ ID NO: 851 - DNA, 138 bp, synthetic construct
  source 1..138: mol_type = other DNA
gcaggcgcgcgacaccacgccgttttagagctagaaatagcaagttaaaataaggctagtccgttatcaacttgaaaaagtggcaccgagtcggtgctgaggtccggagttcaagactaacctggccagtggtgtcgc

SEQ ID NO: 852 - DNA, 143 bp, synthetic construct
  source 1..143: mol_type = other DNA
gcaagactaacctggccaacagttttagagctagaaatagcaagttaaaataaggctagtccgttatcaacttgaaaaagtggcaccgagtcggtgctacaggcgcgcgacaccacatactgggtttcaccatgttggccagg

SEQ ID NO: 853 - DNA, 129 bp, synthetic construct
  source 1..129: mol_type = other DNA
gcaagactaacctggccaacagttttagagctagaaatagcaagttaaaataaggctagtccgttatcaacttgaaaaagtggcaccgagtcggtgctagatactgggtttcaccatgttggccaggtt

SEQ ID NO: 854 - DNA, 147 bp, synthetic construct
  source 1..147: mol_type = other DNA
gtctggagtagctgggattacgttttagagctagaaatagcaagttaaaataaggctagtccgttatcaacttgaaaaagtggcaccgagtcggtgcacatggtgaaacccagtatgtggtgtcgcgcgcctgtaatcccagctact

SEQ ID NO: 855 - DNA, 131 bp, synthetic construct
  source 1..131: mol_type = other DNA
gcaggcgcgcgacaccacgccgttttagagctagaaatagcaagttaaaataaggctagtccgttatcaacttgaaaaagtggcaccgagtcggtgcacatggtgaaacccagtatctagtggtgtcgcgc

SEQ ID NO: 856 - DNA, 129 bp, synthetic construct
  source 1..129: mol_type = other DNA
gcaagactaacctggccaacagttttagagctagaaatagcaagttaaaataaggctagtccgttatcaacttgaaaaagtggcaccgagtcggtgctacaggcgcgcgacaccacgcctggccaggtt

SEQ ID NO: 857 - DNA, 129 bp, synthetic construct
  source 1..129: mol_type = other DNA
gcaagactaacctggccaacagttttagagctagaaatagcaagttaaaataaggctagtccgttatcaacttgaaaaagtggcaccgagtcggtgcgcccggctaacttttctttatggccaggttag

SEQ ID NO: 858 - DNA, 129 bp, synthetic construct
  source 1..129: mol_type = other DNA
gcaagactaacctggccaacagttttagagctagaaatagcaagttaaaataaggctagtccgttatcaacttgaaaaagtggcaccgagtcggtgcacatggtgaaacccagtatctaatcccagcta

SEQ ID NO: 859 - DNA, 129 bp, synthetic construct
  source 1..129: mol_type = other DNA
gcaagactaacctggccaacagttttagagctagaaatagcaagttaaaataaggctagtccgttatcaacttgaaaaagtggcaccgagtcggtgcacatggtgaaacccagtatctagtggtgtcgc

SEQ ID NO: 860 - DNA, 190 bp, synthetic construct
  source 1..190: mol_type = other DNA
gcaagactaacctggccaacagtttaagagctagaaatagcaagtttaaataaggctagtccgttatcaacttgaaaaagtggcaccgagtcggtgctagctgggattacaggcgcgcgacaccacgcccggctaactggccaggcaaataaccgcggttctatctagttacgcgttaaaccaactagaa

SEQ ID NO: 861 - DNA, 192 bp, synthetic construct
  source 1..192: mol_type = other DNA
gcaagactaacctggccaacagtttaagagctagaaatagcaagtttaaataaggctagtccgttatcaacttgaaaaagtggcaccgagtcggtgctagctgggattacaggcgcgcgacaccacgcccggctaactggccaggttcaaataaccgcggttctatctagttacgcgttaaaccaactagaa

SEQ ID NO: 862 - DNA, 194 bp, synthetic construct
  source 1..194: mol_type = other DNA
gcaagactaacctggccaacagtttaagagctagaaatagcaagtttaaataaggctagtccgttatcaacttgaaaaagtggcaccgagtcggtgctagctgggattacaggcgcgcgacaccacgcccggctaactggccaggttagcaaataaccgcggttctatctagttacgcgttaaaccaactagaa

SEQ ID NO: 863 - DNA, 196 bp, synthetic construct
  source 1..196: mol_type = other DNA
gcaagactaacctggccaacagtttaagagctagaaatagcaagtttaaataaggctagtccgttatcaacttgaaaaagtggcaccgagtcggtgctagctgggattacaggcgcgcgacaccacgcccggctaactggccaggttagtccaaataaccgcggttctatctagttacgcgttaaaccaactagaa

SEQ ID NO: 864 - DNA, 145 bp, synthetic construct
  source 1..145: mol_type = other DNA
gcaagactaacctggccaacagtttaagagctagaaatagcaagtttaaataaggctagtccgttatcaacttgaaaaagtggcaccgagtcggtgctagctgggattacaggcgcgcgacaccacgcccggctaactggccagg

SEQ ID NO: 865 - DNA, 147 bp, synthetic construct
  source 1..147: mol_type = other DNA
gcaagactaacctggccaacagtttaagagctagaaatagcaagtttaaataaggctagtccgttatcaacttgaaaaagtggcaccgagtcggtgctagctgggattacaggcgcgcgacaccacgcccggctaactggccaggtt

SEQ ID NO: 866 - DNA, 149 bp, synthetic construct
  source 1..149: mol_type = other DNA
gcaagactaacctggccaacagtttaagagctagaaatagcaagtttaaataaggctagtccgttatcaacttgaaaaagtggcaccgagtcggtgctagctgggattacaggcgcgcgacaccacgcccggctaactggccaggttag

SEQ ID NO: 867 - DNA, 151 bp, synthetic construct
  source 1..151: mol_type = other DNA
gcaagactaacctggccaacagtttaagagctagaaatagcaagtttaaataaggctagtccgttatcaacttgaaaaagtggcaccgagtcggtgctagctgggattacaggcgcgcgacaccacgcccggctaactggccaggttagtc

SEQ ID NO: 868 - DNA, 4789 bp, synthetic construct
  source 1..4789: mol_type = other DNA
gggtaccggcatatggttcttgacagaggtgtaaaaagtactcaaaaattttactcaagtgaaagtacaagtacttagggaaaattttactcaattaaaagtaaaagtatctggctagaatcttacttgagtaaaagtaaaaaagtactccattaaaattgtacttgagtattaaggaagtaaaagtaaaagcaagaaagatcgatctcgaaggatctggaggccaccatggtgtcgataacttcgtatagcatacattatacgaagttatcgtgctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcctcgagatggcgaccctggaaaagctgatgaaggccttcgagtccctcaagtccttccagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcaacagccgccaccgccgccgccgccgccgccgcctcctcagcttcctcagccgccgggatccgctagcgctactaacttcagcctgctgaagcaggctggagacgtggaggagaaccctggacctggaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgccaccgcggtggagctcgaattaattcatcgatgatgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagagtcggtgggcctcgggggcgggtgcggggtcggcggggccgccccgggtggcttcggtcggagccatggggtcgtgcgctcctttcggtcgggcgctgcgggtcgtggggcgggcgtcaggcaccgggcttgcgggtcatgcaccaggtgcgcggtccttcgggcacctcgacgtcggcggtgacggtgaagccgagccgctcgtagaaggggaggttgcggggcgcggaggtctccaggaaggcgggcaccccggcgcgctcggccgcctccactccggggagcacgacggcgctgcccagacccttgccctggtggtcgggcgagacgccgacggtggccaggaaccacgcgggctccttgggccggtgcggcgccaggaggccttccatctgttgctgcgcggccagccgggaaccgctcaactcggccatgcgcgggccgatctcggcgaacaccgcccccgcttcgacgctctccggcgtggtccagaccgccaccgcggcgccgtcgtccgcgacccacaccttgccgatgtcgagcccgacgcgcgtgaggaagagttcttgcagctcggtgacccgctcgatgtggcggtccgggtcgacggtgtggcgcgtggcggggtagtcggcgaacgcggcggcgagggtgcgtacggcccgggggacgtcgtcgcgggtggcgaggcgcaccgtgggcttgtactcggtcatggaaggtcgtctccttgtgaggggtcaggggcgtgggtcaggggatggtggcggcaccggtcgtggcggccgacctgcaggcatgcaagctttttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagaattcaagtgatctccaaaaaataagtactttttgactgtaaataaaattgtaaggagtaaaaagtacttttttttctaaaaaaatgtaattaagtaaaagtaaaagtattgatttttaattgtactcaagtaaagtaaaaatccccaaaaataatacttaagtacagtaatcaagtaaaattactcaagtactttacacctctgg

SEQ ID NO: 869 - DNA, 4636 bp, synthetic construct
  source 1..4636: mol_type = other DNA
gggtaccggcatatggttcttgacagaggtgtaaaaagtactcaaaaattttactcaagtgaaagtacaagtacttagggaaaattttactcaattaaaagtaaaagtatctggctagaatcttacttgagtaaaagtaaaaaagtactccattaaaattgtacttgagtattaaggaagtaaaagtaaaagcaagaaagatcgatctcgaaggatctggaggccaccatggtgtcgataacttcgtatagcatacattatacgaagttatcgtgctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcctcgagatggcgaccctggaaaagctgatgaaggccttcgagtccctcaagtccttccagcagcagcagcagcagcagcagcagcagcagcagcagcagcagcagcagcagcagcagcagcaacagccgccaccgccgccgccgccgccgccgcctcctcagcttcctcagccgccgggatccgctagcgctactaacttcagcctgctgaagcaggctggagacgtggaggagaaccctggacctggaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgccaccgcggtggagctcgaattaattcatcgatgatgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagagtcggtgggcctcgggggcgggtgcggggtcggcggggccgccccgggtggcttcggtcggagccatggggtcgtgcgctcctttcggtcgggcgctgcgggtcgtggggcgggcgtcaggcaccgggcttgcgggtcatgcaccaggtgcgcggtccttcgggcacctcgacgtcggcggtgacggtgaagccgagccgctcgtagaaggggaggttgcggggcgcggaggtctccaggaaggcgggcaccccggcgcgctcggccgcctccactccggggagcacgacggcgctgcccagacccttgccctggtggtcgggcgagacgccgacggtggccaggaaccacgcgggctccttgggccggtgcggcgccaggaggccttccatctgttgctgcgcggccagccgggaaccgctcaactcggccatgcgcgggccgatctcggcgaacaccgcccccgcttcgacgctctccggcgtggtccagaccgccaccgcggcgccgtcgtccgcgacccacaccttgccgatgtcgagcccgacgcgcgtgaggaagagttcttgcagctcggtgacccgctcgatgtggcggtccgggtcgacggtgtggcgcgtggcggggtagtcggcgaacgcggcggcgagggtgcgtacggcccgggggacgtcgtcgcgggtggcgaggcgcaccgtgggcttgtactcggtcatggaaggtcgtctccttgtgaggggtcaggggcgtgggtcaggggatggtggcggcaccggtcgtggcggccgacctgcaggcatgcaagctttttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagaattcaagtgatctccaaaaaataagtactttttgactgtaaataaaattgtaaggagtaaaaagtacttttttttctaaaaaaatgtaattaagtaaaagtaaaagtattgatttttaattgtactcaagtaaagtaaaaatccccaaaaataatacttaagtacagtaatcaagtaaaattactcaagtactttacacctctgg

SEQ ID NO: 870 - DNA, 4785 bp, synthetic construct
  source 1..4785: mol_type = other DNA
gggtaccggcatatggttcttgacagaggtgtaaaaagtactcaaaaattttactcaagtgaaagtacaagtacttagggaaaattttactcaattaaaagtaaaagtatctggctagaatcttacttgagtaaaagtaaaaaagtactccattaaaattgtacttgagtattaaggaagtaaaagtaaaagcaagaaagatcgatctcgaaggatctggaggccaccatggtgtcgataacttcgtatagcatacattatacgaagttatcgtgctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcctcgagatggcgaccctggaaaagctgatgaaggccttcgagtccctcaagtccttccagcagcagcagcagccgcagcagcagcagcagcagcagcagcagcagcagcagcagcagcagcagcagcagcagcagcagcagcagcagcagcagccgcagcagcagcagcagcagcagcagcagcagcagccgcagcagcagcagcagcagcagcagcagcagcagcagcagcagcagcagcagcagcagcagcagcagcagcagcagcagcaacagccgccaccgccgccgccgccgccgccgcctcctcagcttcctcagccgccgggatccgctagcgctactaacttcagcctgctgaagcaggctggagacgtggaggagaaccctggacctggaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgccaccgcggtggagctcgaattaattcatcgatgatgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagagtcggtgggcctcgggggcgggtgcggggtcggcggggccgccccgggtggcttcggtcggagccatggggtcgtgcgctcctttcggtcgggcgctgcgggtcgtggggcgggcgtcaggcaccgggcttgcgggtcatgcaccaggtgcgcggtccttcgggcacctcgacgtcggcggtgacggtgaagccgagccgctcgtagaaggggaggttgcggggcgcggaggtctccaggaaggcgggcaccccggcgcgctcggccgcctccactccggggagcacgacggcgctgcccagacccttgccctggtggtcgggcgagacgccgacggtggccaggaaccacgcgggctccttgggccggtgcggcgccaggaggccttccatctgttgctgcgcggccagccgggaaccgctcaactcggccatgcgcgggccgatctcggcgaacaccgcccccgcttcgacgctctccggcgtggtccagaccgccaccgcggcgccgtcgtccgcgacccacaccttgccgatgtcgagcccgacgcgcgtgaggaagagttcttgcagctcggtgacccgctcgatgtggcggtccgggtcgacggtgtggcgcgtggcggggtagtcggcgaacgcggcggcgagggtgcgtacggcccgggggacgtcgtcgcgggtggcgaggcgcaccgtgggcttgtactcggtcatggaaggtcgtctccttgtgaggggtcaggggcgtgggtcaggggatggtggcggcaccggtcgtggcggccgacctgcaggcatgcaagctttttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagaattcaagtgatctccaaaaaataagtactttttgactgtaaataaaattgtaaggagtaaaaagtacttttttttctaaaaaaatgtaattaagtaaaagtaaaagtattgatttttaattgtactcaagtaaagtaaaaatccccaaaaataatacttaagtacagtaatcaagtaaaattactcaagtactttacacctctg

SEQ ID NO: 871 - DNA, 4787 bp, synthetic construct
  source 1..4787: mol_type = other DNA
gggtaccggcatatggttcttgacagaggtgtaaaaagtactcaaaaattttactcaagtgaaagtacaagtacttagggaaaattttactcaattaaaagtaaaagtatctggctagaatcttacttgagtaaaagtaaaaaagtactccattaaaattgtacttgagtattaaggaagtaaaagtaaaagcaagaaagatcgatctcgaaggatctggaggccaccatggtgtcgataacttcgtatagcatacattatacgaagttatcgtgctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcctcgagatggcgaccctggaaaagctgatgaaggccttcgagtccctcaagtccttccagcagcagcagcagcagcagcaacagcagcagcagcagcagcagcaacagcagcagcagcagcagcagcagcagcagcagcagcagcagcagcagcagcagcagcagcagcagcagcagcaacagcagcagcagcagcagcagcagcagcagcagcagcagcagcagcagcagcagcagcagcagcagcagcaacagcagcagcagcagcagcagcaacagccgccaccgccgccgccgccgccgccgcctcctcagcttcctcagccgccgggatccgctagcgctactaacttcagcctgctgaagcaggctggagacgtggaggagaaccctggacctggaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgccaccgcggtggagctcgaattaattcatcgatgatgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagagtcggtgggcctcgggggcgggtgcggggtcggcggggccgccccgggtggcttcggtcggagccatggggtcgtgcgctcctttcggtcgggcgctgcgggtcgtggggcgggcgtcaggcaccgggcttgcgggtcatgcaccaggtgcgcggtccttcgggcacctcgacgtcggcggtgacggtgaagccgagccgctcgtagaaggggaggttgcggggcgcggaggtctccaggaaggcgggcaccccggcgcgctcggccgcctccactccggggagcacgacggcgctgcccagacccttgccctggtggtcgggcgagacgccgacggtggccaggaaccacgcgggctccttgggccggtgcggcgccaggaggccttccatctgttgctgcgcggccagccgggaaccgctcaactcggccatgcgcgggccgatctcggcgaacaccgcccccgcttcgacgctctccggcgtggtccagaccgccaccgcggcgccgtcgtccgcgacccacaccttgccgatgtcgagcccgacgcgcgtgaggaagagttcttgcagctcggtgacccgctcgatgtggcggtccgggtcgacggtgtggcgcgtggcggggtagtcggcgaacgcggcggcgagggtgcgtacggcccgggggacgtcgtcgcgggtggcgaggcgcaccgtgggcttgtactcggtcatggaaggtcgtctccttgtgaggggtcaggggcgtgggtcaggggatggtggcggcaccggtcgtggcggccgacctgcaggcatgcaagctttttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagaattcaagtgatctccaaaaaataagtactttttgactgtaaataaaattgtaaggagtaaaaagtacttttttttctaaaaaaatgtaattaagtaaaagtaaaagtattgatttttaattgtactcaagtaaagtaaaaatccccaaaaataatacttaagtacagtaatcaagtaaaattactcaagtactttacacctct

SEQ ID NO: 872 - DNA, 5079 bp, synthetic construct
  source 1..5079: mol_type = other DNA
ggtaccggcatatggttcttgacagaggtgtaaaaagtactcaaaaattttactcaagtgaaagtacaagtacttagggaaaattttactcaattaaaagtaaaagtatctggctagaatcttacttgagtaaaagtaaaaaagtactccattaaaattgtacttgagtattaaggaagtaaaagtaaaagcaagaaagatcgatctcgaaggatctggaggccaccatggtgtcgataacttcgtatagcatacattatacgaagttatcgtgctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcctcgagcggccgccagtgtgatggatatcggatcaggcttgaacttcccacacgtgttatttggcccacattgtgtttgaagaaactttgggattggttgccagtgcttaaaagttaggacttagaaaatggatttcctggcaggacgcggtggctcatgcccataatctcagcactttgggaggcctaggaaggtggatcacctgaggtccggagttcaagactaacctggccaacatggtgaaacccagtatctactaaaaaatacaaaaaaaaaaaaaaaagaagaagaagaagaagaagaagaagaagaagaagaagaagaagaagaagaagaagaagaagaagaagaagaagaagaagaagaagaagaaaataaagaaaagttagccgggcgtggtgtcgcgcgcctgtaatcccagctactccagaggctgcggcaggagaatcgcttgagcccgggaggcagaggttgcattaagccaagatcgcccaatgcactccggcctgggcgacagagcaagactccgtctcaaaaaataataataataaataaaaataaaaaataaaatggatttcccagcatctctggaaaaataggcaagtgtggccatgatggtcctgatccgctagcgctactaacttcagcctgctgaagcaggctggagacgtggaggagaaccctggacctggaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgccaccgcggtggagctcgaattaattcatcgatgatgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagagtcggtgggcctcgggggcgggtgcggggtcggcggggccgccccgggtggcttcggtcggagccatggggtcgtgcgctcctttcggtcgggcgctgcgggtcgtggggcgggcgtcaggcaccgggcttgcgggtcatgcaccaggtgcgcggtccttcgggcacctcgacgtcggcggtgacggtgaagccgagccgctcgtagaaggggaggttgcggggcgcggaggtctccaggaaggcgggcaccccggcgcgctcggccgcctccactccggggagcacgacggcgctgcccagacccttgccctggtggtcgggcgagacgccgacggtggccaggaaccacgcgggctccttgggccggtgcggcgccaggaggccttccatctgttgctgcgcggccagccgggaaccgctcaactcggccatgcgcgggccgatctcggcgaacaccgcccccgcttcgacgctctccggcgtggtccagaccgccaccgcggcgccgtcgtccgcgacccacaccttgccgatgtcgagcccgacgcgcgtgaggaagagttcttgcagctcggtgacccgctcgatgtggcggtccgggtcgacggtgtggcgcgtggcggggtagtcggcgaacgcggcggcgagggtgcgtacggcccgggggacgtcgtcgcgggtggcgaggcgcaccgtgggcttgtactcggtcatggaaggtcgtctccttgtgaggggtcaggggcgtgggtcaggggatggtggcggcaccggtcgtggcggccgacctgcaggcatgcaagctttttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagaattcaagtgatctccaaaaaataagtactttttgactgtaaataaaattgtaaggagtaaaaagtacttttttttctaaaaaaatgtaattaagtaaaagtaaaagtattgatttttaattgtactcaagtaaagtaaaaatccccaaaaataatacttaagtacagtaatcaagtaaaattactcaagtactttacacctctg

SEQ ID NO: 873 - DNA, 18 bp, synthetic construct
  source 1..18: mol_type = other DNA
gaccctggaaaagctgat

SEQ ID NO: 874 - DNA, 12 bp, synthetic construct
  source 1..12: mol_type = other DNA
cagcagcagcag

SEQ ID NO: 875 - DNA, 27 bp, synthetic construct
  source 1..27: mol_type = other DNA
cagcagcagcagcagcagcagcagcag

SEQ ID NO: 876 - DNA, 21 bp, synthetic construct
  source 1..21: mol_type = other DNA
cagcagcagcagcaacaacaa

SEQ ID NO: 877 - DNA, 86 bp, Homo sapiens
  source 1..86: mol_type = genomic DNA
tccctcaagtcttcgagtccctcaagtccttccagcagcagcagcagcagcagcagcagcagcagcagcaacagccgccaccgccg

SEQ ID NO: 878 - DNA, 13 bp, synthetic construct
  source 1..13: mol_type = other DNA
cttccagcaacag

SEQ ID NO: 879 - DNA, 13 bp, synthetic construct
  source 1..13: mol_type = other DNA
cttccagcaacag

SEQ ID NO: 880 - DNA, 13 bp, Homo sapiens
  source 1..13: mol_type = genomic DNA
cttccagcaacag

SEQ ID NO: 881 - DNA, 13 bp, synthetic construct
  source 1..13: mol_type = other DNA
cttccagcaacag

SEQ ID NO: 882 - DNA, 76 bp, synthetic construct
  source 1..76: mol_type = other DNA
gttttagagctagaaatagcaagttaaaataaggctagtccgttatcaacttgaaaaagtggcaccgagtcggtgc

SEQ ID NO: 883 - DNA, 76 bp, synthetic construct
  source 1..76: mol_type = other DNA
gtttaagagctagaaatagcaagtttaaataaggctagtccgttatcaacttgaaaaagtggcaccgagtcggtgc

SEQ ID NO: 884 - DNA, 83 bp, synthetic construct
  source 1..83: mol_type = other DNA
atggcgaccctggaaaagctgatgaaggccttcgagtccctcaagtccttccagcagcagcagcagcagcagcagcagcagca

SEQ ID NO: 885 - protein, 28 aa, synthetic construct
MATLEKLMKAFESLKSFQQQQQQQQQQQ

SEQ ID NO: 886 - DNA, 83 bp, synthetic construct
  source 1..83: mol_type = other DNA
tgctgctgctgctgctgctgctgctgctgctggaaggacttgagggactcgaaggccttcatcagcttttccagggtcgccat

SEQ ID NO: 887 - DNA, 27 bp, synthetic construct
  source 1..27: mol_type = other DNA
cagcagcagcagcagcagcagcaacag

SEQ ID NO: 888 - DNA, 18 bp, synthetic construct
  source 1..18: mol_type = other DNA
cagcagcagcagcaacag

SEQ ID NO: 889 - DNA, 18 bp, synthetic construct
  source 1..18: mol_type = other DNA
cagcagcaacagcaacag

SEQ ID NO: 890 - DNA, 18 bp, synthetic construct
  source 1..18: mol_type = other DNA
caacagcaacagcaacag

SEQ ID NO: 891 - DNA, 18 bp, synthetic construct
  source 1..18: mol_type = other DNA
caacaacaacaacaacag

SEQ ID NO: 892 - DNA, 90 bp, synthetic construct
  source 1..90: mol_type = other DNA
atggcgaccctggaaaagctgatgaaggccttcgagtccctcaagtccttccagcagcagcagcagcagcagcagcagcagcagcagcag

SEQ ID NO: 893 - protein, 30 aa, synthetic construct
MATLEKLMKAFESLKSFQQQQQQQQQQQQQ

SEQ ID NO: 894 - DNA, 90 bp, synthetic construct
  source 1..90: mol_type = other DNA
ctgctgctgctgctgctgctgctgctgctgctgctgctggaaggacttgagggactcgaaggccttcatcagcttttccagggtcgccat

SEQ ID NO: 895 - DNA, 274 bp, synthetic construct
  source 1..274: mol_type = other DNA
catggcgaccctggaaaagctgatgaaggccttcgagtccctcaagtccttccagcagcagcagcagcagcagcagcagcagcagcagcagcagcagcagcagcagcagcaacagccgccaccgccgccgccgccgccgccgcctcctcagcttcctcagccgccgccgcaggcacagccgctgctgcctcagccgcagccgcccccgccgccgcccccgccgccacccggcccggctgtggctgaggagccgctgcaccgaccgtgagtttgg

SEQ ID NO: 896 - protein, 91 aa, synthetic construct
MATLEKLMKAFESLKSFQQQQQQQQQQQQQQQQQQQQQPPPPPPPPPPPQLPQPPPQAQPLLPQPQPPPPPPPPPPGPAVAEEPLHRPXVW

SEQ ID NO: 897 - DNA, 274 bp, synthetic construct
  source 1..274: mol_type = other DNA
ccaaactcacggtcggtgcagcggctcctcagccacagccgggccgggtggcggcgggggcggcggcgggggcggctgcggctgaggcagcagcggctgtgcctgcggcggcggctgaggaagctgaggaggcggcggcggcggcggcggcggtggcggctgttgctgctgctgctgctgctgctgctgctgctgctgctgctgctgctgctgctgctgctggaaggacttgagggactcgaaggccttcatcagcttttccagggtcgccatg

SEQ ID NO: 898 - DNA, 61 bp, synthetic construct
  source 1..61: mol_type = other DNA
cttccagcagcagcagcagcagcagcagcagcagcagcagcagcagcagcagcagcaacag

SEQ ID NO: 899 - DNA, 22 bp, synthetic construct
  source 1..22: mol_type = other DNA
cttccagcagcagcagcaacag

SEQ ID NO: 900 - DNA, 57 bp, synthetic construct
  source 1..57: mol_type = other DNA
cagcagcagcagcagcagcagcagcagcagcagcagcagcagcagcagcagcaacag

SEQ ID NO: 901 - DNA, 54 bp, synthetic construct
  source 1..54: mol_type = other DNA
cagcagcagcagcagcagcagcagcagcagcagcagcagcagcagcagcaacag

SEQ ID NO: 902 - DNA, 87 bp, synthetic construct
  source 1..87: mol_type = other DNA
cagcagcagcagcagcagcagcagcagcagcagcagcagcagcagcagcagcagcagcagcagcagcagcagcagcagcagcaacag

SEQ ID NO: 903 - DNA, 69 bp, synthetic construct
  source 1..69: mol_type = other DNA
gatctactaaaaaatacaaaaaaaaaaaaaaaagaagaagaagaagaaaataaagaaaagttagccggg

SEQ ID NO: 904 - DNA, 54 bp, synthetic construct
  source 1..54: mol_type = other DNA
gatctactaaaaaatacaaaaaaaaaaaaaaaaaataaagaaaagttagccggg

SEQ ID NO: 905 - DNA, 84 bp, Homo sapiens
  source 1..84: mol_type = genomic DNA
gatctactaaaaaatacaaaaaaaaaaaaaaaagaagaagaagaagaagaagaagaagaagaaaataaagaaaagttagccggg

SEQ ID NO: 906 - DNA, 239 bp, synthetic construct
  source 1..239: mol_type = other DNA
ggatttcctggcaggacgcggtggctcatgcccataatctcagcactttgggaggcctaggaaggtggatcacctgaggtccggagttcaagactaacctggccaacatggtgaaacccagtatctactaaaaaatacaaaaaaaaaaaaaaaagaagaagaagaagaagaaaataaagaaaagttagccgggcgtggtgtcgcgcgcctgtaatcccagctactccagaggctgcggc

SEQ ID NO: 907 - DNA, 239 bp, synthetic construct
  source 1..239: mol_type = other DNA
gccgcagcctctggagtagctgggattacaggcgcgcgacaccacgcccggctaacttttctttattttcttcttcttcttcttcttttttttttttttttgtattttttagtagatactgggtttcaccatgttggccaggttagtcttgaactccggacctcaggtgatccaccttcctaggcctcccaaagtgctgagattatgggcatgagccaccgcgtcctgccaggaaatcc

SEQ ID NO: 908 - DNA, 259 bp, synthetic construct
  source 1..259: mol_type = other DNA
aggtccggagttcaagactaacctggccaacatggtgaaacccagtatctactaaaaaatacaaaaaaaaaaaaaaaagaagaagaagaagaagaaaataaagaaaagttagccgggcgtggtgtcgcgcgcctgtaatcccagctactccagaggctgcggcaggagaatcgcttgagcccgggaggcagaggttgcattaagccaagatcgcccaatgcactccggcctgggcgacagagcaagactccgtctcaaa

SEQ ID NO: 909 - DNA, 259 bp, synthetic construct
  source 1..259: mol_type = other DNA
tttgagacggagtcttgctctgtcgcccaggccggagtgcattgggcgatcttggcttaatgcaacctctgcctcccgggctcaagcgattctcctgccgcagcctctggagtagctgggattacaggcgcgcgacaccacgcccggctaacttttctttattttcttcttcttcttcttcttttttttttttttttgtattttttagtagatactgggtttcaccatgttggccaggttagtcttgaactccggacct

SEQ ID NO: 910 - DNA, 142 bp, synthetic construct
  source 1..142: mol_type = other DNA
agttcaagactaacctggccaacatggtgaaacccagtatctactaaaaaatacaaaaaaaaaaaaaaaagaagaagaagaagaagaaaataaagaaaagttagccgggcgtggtgtcgcgcgcctgtaatcccagctactc

SEQ ID NO: 911 - DNA, 142 bp, synthetic construct
  source 1..142: mol_type = other DNA
tcaagttctgattggaccggttgtaccactttgggtcatagatgattttttatgttttttttttttttttcttcttcttcttcttcttttatttcttttcaatcggcccgcaccacagcgcgcggacattagggtcgatgag

SEQ ID NO: 912 - DNA, 74 bp, synthetic construct
  source 1..74: mol_type = other DNA
tggtgaaacccagtatctactaaaaaatacaaaaaaaaaaaaaaaagaagaagaagaaaataaagaaaagttag

SEQ ID NO: 913 - DNA, 17 bp, synthetic construct
  source 1..17: mol_type = other DNA
tggtgaaacccagttag

SEQ ID NO: 914 - DNA, 27 bp, synthetic construct
  source 1..27: mol_type = other DNA
gaagaagaagaagaagaagaagaagaa

SEQ ID NO: 915 - DNA, 24 bp, synthetic construct
  source 1..24: mol_type = other DNA
gaagaagaagaagaagaagaagaa

SEQ ID NO: 916 - DNA, 54 bp, synthetic construct
  source 1..54: mol_type = other DNA
gaagaagaagaagaagaagaagaagaagaagaagaagaagaagaagaagaagaa

SEQ ID NO: 917 - DNA, 48 bp, synthetic construct
  source 1..48: mol_type = other DNA
gaagaagaagaagaagaagaagaagaagaagaagaagaagaagaagaa

SEQ ID NO: 918 - DNA, 140 bp, synthetic construct
  source 1..140: mol_type = other DNA
agttcaagactaacctggccaacatggtgaaacccagtatctactaaaaaatacaaaaaaaaaaaaaaaagaagaagaagaagaagaaaataaagaaaagttagccgggcgtggtgtcgcgcctgtaatcccagctactc

SEQ ID NO: 919 - DNA, 140 bp, synthetic construct
  source 1..140: mol_type = other DNA
gagtagctgggattacaggcgcgacaccacgcccggctaacttttctttattttcttcttcttcttcttcttttttttttttttttgtattttttagtagatactgggtttcaccatgttggccaggttagtcttgaact

SEQ ID NO: 920 - DNA, 259 bp, synthetic construct
  source 1..259: mol_type = other DNA
aggtccggagttcaagactaacctggccaacatggtgaaacccagtatctactaaaaaatacaaaaaaaaaaaaaaagaagaagaagaagaagaaaataaaggaaaagttagccgggcgtggtgtcgcgcgcctgtaatcccagctactccagaggctgcggcaggagaatcgcttgagcccgggaggcagaggttgcattaagccaagatcgcccaatgcactccggcctgggcgacagagcaagactccgtctcaaa

SEQ ID NO: 921 - DNA, 259 bp, synthetic construct
  source 1..259: mol_type = other DNA
tttgagacggagtcttgctctgtcgcccaggccggagtgcattgggcgatcttggcttaatgcaacctctgcctcccgggctcaagcgattctcctgccgcagcctctggagtagctgggattacaggcgcgcgacaccacgcccggctaacttttcctttattttcttcttcttcttcttctttttttttttttttgtattttttagtagatactgggtttcaccatgttggccaggttagtcttgaactccggacct

SEQ ID NO: 922 - DNA, 252 bp, synthetic construct
  source 1..252: mol_type = other DNA
gatctactaaaaaatacaaaaaaaaaaaaaaaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaaaataaagaaaagttagccggg

SEQ ID NO: 923 - DNA, 238 bp, synthetic construct
  source 1..238: mol_type = other DNA
ggatttcctggcaggacgcggtggctcatgcccataatctcagcactttgggaggcctaggaaggtggatcacctgaggtccggagttcaagactaacctggccaacatggtgaaacccagtatctactaaaaaatacaaaaaaaaaaaaaaaagaagaagaagaagaagaaaataaagaaaagttagccgggcgtggtgtcgcgcgcctgtaatcccagctactccagaggctgcgg

SEQ ID NO: 924 - DNA, 234 bp, synthetic construct
  source 1..234: mol_type = other DNA
ccgcagcctctggagtagctgggattacaggcgcgcgacaccacgcccggctaacttttctttattttcttcttcttcttctttttttttttttttgtattttttagtagatactgggtttcaccatgttggccaggttagtcttgaactccggacctcaggtgatccaccttcctaggcctcccaaagtgctgagattatgggcatgagccaccgcgtcctgccaggaaatcc

SEQ ID NO: 925 - DNA, 260 bp, synthetic construct
  source 1..260: mol_type = other DNA
tggtgaaacccagtatctactaaaaaatacaaaaaaaaaaaaaaaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaagaaaataaagaaaagttag

SEQ ID NO: 926 - DNA, 241 bp, synthetic construct
  source 1..241: mol_type = other DNA
atgaaagccttcgaatccctgaagtcctttcagcagcagcagcaacagccgccaccgccgccgccgcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagg

SEQ ID NO: 927 - DNA, 134 bp, synthetic construct
  source 1..134: mol_type = other DNA
atgaaagccttcgaatccctgaagtcctttcagcagcagcagcaacagccgccaccgccgccgccgccgccgccgcctcctcagcttcctcagccgccgccgcaggcacagccgctgctgcctcagccgcagcc

(19) United States
(12) Patent Application Publication
(10) Pub. No.: US 2026/0364201 A1
(43) Pub. Date: Nov. 19, 2026

(54) PRIME EDITING METHODS AND COMPOSITIONS FOR TREATING TRIPLET REPEAT DISORDERS

(71) Applicants: The Broad Institute, Inc., Cambridge, MA (US); President and Fellows of Harvard College, Cambridge, MA (US)

(72) Inventors: David R. Liu; Zaneta Matuszek, Cambridge, MA (US); Mandana Arbab, Cambridge, MA (US)

(73) Assignees: The Broad Institute, Inc., Cambridge, MA (US); President and Fellows of Harvard College, Cambridge, MA (US)

(21) Appl. No.: 19/118,760

(22) PCT Filed: Oct. 6, 2023
(86) PCT No.: PCT/US2023/076282

§ 371 Date: Apr. 4, 2025

Related U.S. Application Data
(60) Provisional application No. 63/414,364, filed on Oct. 7, 2022.
(60) Provisional application No. 63/508,616, filed on Jun. 16, 2023.

(51) Int. Cl. A61K 48/00; A61K 9/00; A61P 25/28; C12N 9/12; C12N 9/22; C12N 15/11; C12N 15/86

(52) U.S. Cl. CPC A61K 48/005; A61K 9/0085; A61K 48/0075; A61P 25/28; C12N 9/1276; C12N 9/226; C12N 15/111; C12N 15/86; C07K 2319/80; C12N 2310/20; C12N 2750/14143; C12N 2800/40; C12Y 207/07049

(57) ABSTRACT

The present disclosure provides compositions and methods useful in the treatment of trinucleotide repeat disorders, including Huntington's disease and Friedreich's ataxia. The present disclosure also provides pegRNAs designed to target the HTT or FXN genes. Complexes, compositions, and systems comprising a prime editor and any of the pegRNAs disclosed herein are also provided by the present disclosure. The present disclosure further provides polynucleotides, vectors, AAVs, cells, compositions, and kits. Methods of treating Huntington's disease and Friedreich's ataxia, as well as uses of the compositions, pegRNAs, and systems described herein, are also provided herein.

### RELATED APPLICATIONS

This application claims priority under 35 U.S.C. § 119(e) to U.S. Provisional Application, U.S. Ser. No. 63/414,364, filed Oct. 7, 2022, and U.S. Provisional Application, U.S. Ser. No. 63/508,616, filed Jun. 16, 2023, each of which is incorporated herein by reference.

### GOVERNMENT SUPPORT

This invention was made with government support under grant numbers U01AI142756, RM1HG009490, R01EB031172, and R35GM118062, awarded by the National Institutes of Health. The government has certain right in the invention.

### BACKGROUND OF THE INVENTION

Triplet repeat disorders, including Huntington's disease (HD) and Friedreich's Ataxia (FRDA), are complex, progressive disorders that involve developmental neurobiology and often affect cognition as well as sensory-motor functions. The disorders show genetic anticipation (i.e., increased severity with each generation), and the DNA expansions or contractions usually happen meiotically (i.e., during the time of gametogenesis, or early in embryonic development) and often have sex-bias, meaning that some genes expand only when inherited through the female and others only through the male. The DNA expansions and contractions can also happen somatically (i.e., during an individual's development or lifetime). In humans, trinucleotide repeat expansion disorders can cause gene silencing at either the transcriptional or translational level, which essentially knocks out gene function. Alternatively, trinucleotide repeat expansion disorders can cause altered proteins generated with large repetitive amino acid sequences that either abrogate or change protein function, often in a dominant-negative manner (e.g., poly-glutamine diseases).

Huntington's Disease is an autosomal dominant disorder characterized by the loss of striatal neurons in the central nervous system and is associated with progressive unwanted choreatic movements, behavioral and psychiatric disturbances, and dementia. HD is caused by CAG triplet repeat expansions in the first exon of the HTT gene, which codes for huntingtin protein, resulting in an expanded stretch of glutamines (polyQ). There is no cure or effective treatment for HD, and while some therapeutic interventions may lessen the severity of patient symptoms, HD typically results in fatality within 10-30 years of disease onset. There are no available treatments that can alter the course of the disease.

The age of HD onset in patients and severity of symptoms are inversely correlated with CAG repeat length of pathogenic HTT alleles encoding poly-glutamine (polyQ), with larger alleles generally associated with earlier disease onset and more severe clinical phenotypes. These repeat lengths range between 9-35 in the general population, while HD patients typically carry 40-50 repeats from birth. Individuals with an intermediate range of 36 to 39 CAGs may develop HD at later stages, with lower penetrance and variable clinical manifestation. Notably, CAG repeat length correlates with repeat instability, and long, unstable CAG repeats undergo somatic expansion in some tissues throughout a patient's life, particularly including tissues in the central nervous system (CNS).

The identification of long, unstable polyQ in HTT as causal to HD presents a potentially actionable target for gene-based therapies. Prior studies have employed Cas9 and zinc finger nucleases (ZFNs) in cell and animal models to either excise the region in HTT exon 1 containing the polyQ region, or to create double-strand breaks (DSBs) or nicks within CAG repeats with the aim of inducing contraction of the polyQ. However, nuclease-mediated DSB formation in long CAG repeats results in an almost equal number of allele expansions as contractions. Moreover, nuclease activity at CAG repeats induces a mixture of genomic outcomes at HTT alleles that includes indels, nonsense mutations, and protein truncation, of which the biological consequence is either unknown or deleterious.

Friedreich's Ataxia is an autosomal recessive disorder characterized by progressive ataxia and damage to the nervous system and is often associated with muscle weakness, spasticity, cardiomyopathy, and diabetes mellitus. FRDA is the most common hereditary ataxia in the United States, Europe, the Middle East, South Asia (Indian subcontinent), and North Africa, with a carrier frequency between 1:60-1:100 individuals, though it is rarely identified in other populations. FRDA is typically caused by the expansion of a GAA-triplet repeat in intron 1 of the FXN gene, resulting in transcriptional silencing and deficiency in frataxin (FXN) protein levels to below 30% of normal.

The age of FRDA onset in patients, loss of FXN protein, and severity of symptoms are inversely correlated with the GAA repeat length of the shortest FXN allele. The length of FXN GAA-repeats in the general population ranges from ~5-60, while FRDA patients may present with 66 to well over 1200 repeats, typically ranging from 600 to 900 repeats. Notably, GAA repeat length correlates with repeat instability, and long, unstable GAA repeats undergo somatic expansion in some tissues throughout a patient's life that are particularly affected in FRDA, including the dorsal root ganglia (DRGs), spinal cord, cerebellum, heart, and pancreas, that subsequently experience greater loss of FXN protein expression.

The identification of long, unstable GAA repeats in FXN alleles as causal to FRDA presents a potentially actionable target for gene-based therapies. Prior studies have used dual Cas9 and zinc finger nucleases (ZFNs) flanking GAA repeat loci to induce double strand breaks (DSBs) that resulted in the deletion of the repeat locus, and consequently the upregulation of FXN protein levels and correction of some disease phenotypes in FRDA patient-derived cell lines. However, DSB formation in the genome can severely impact genomic and cellular integrity by inducing large genomic rearrangements and aneuploidy, and Cas9-nucleases have been demonstrated to cause activation of p53 in targeted cells. Moreover, the historic Alu elements that contain GAA repeats, such as in FXN, are frequent in the genome, and nuclease targeting of GAA flanking sequences within these common regions is therefore likely to induce DSBs throughout the genome, while nuclease targeting outside of these common regions to increase specificity results in deletion of larger (1-20 kb) regions of FXN intron 1 that includes critical regulatory domains of FXN expression. Direct nuclease targeting or Cas9 nicking of long GAA repeats has not been explored. GAA repeat expansion at long FXN alleles in FRDA is thought, however, to arise from DSB and gap-formation in GAA repeats that result from DNA nicks arising from secondary structure formation at these loci. Thus, nuclease and nicking activity within or flanking repeat loci does not enable reliable correction of FXN expression, and the biological consequences of unintended nuclease and nicking activity are not entirely known and may be deleterious.

A more precise gene-based therapy is needed to convert pathogenic HTT and FXN alleles to wild type alleles.

### SUMMARY OF THE INVENTION

The present disclosure describes the use of prime editing to reduce the size of CAG repeat tracts to a normal polyQ length in cell and animal models (for example, by AAV delivery), as well as in subjects being treated, that contain pathogenic HTT alleles, without further changes to the flanking coding sequence. In certain embodiments, a CAG repeat sequence is contracted to approximately four CAG repeats in length (e.g., approximately 10 CAG repeats in length, approximately 9 CAG repeats in length, approximately 8 CAG repeats in length, approximately 7 CAG repeats in length, approximately 6 CAG repeats in length, approximately 5 CAG repeats in length, approximately 4 CAG repeats in length, or approximately 3 CAG repeats in length). In certain embodiments, a CAG repeat sequence is replaced with a CAG repeat sequence of approximately four CAG repeats in length (e.g., approximately 10 CAG repeats in length, approximately 9 CAG repeats in length, approximately 8 CAG repeats in length, approximately 7 CAG repeats in length, approximately 6 CAG repeats in length, approximately 5 CAG repeats in length, approximately 4 CAG repeats in length, or approximately 3 CAG repeats in length). The present disclosure also describes the use of prime editing to remove long GAA repeats at FXN alleles in cell and animal models that contain pathogenic FXN alleles, with minimal loss of the surrounding FXN regulatory region in intron 1. In certain embodiments, a GAA sequence of approximately 65 GAA repeats in length is deleted (e.g., approximately 60 GAA repeats in length, approximately 61 GAA repeats in length, approximately 62 GAA repeats in length, approximately 63 GAA repeats in length, approximately 64 GAA repeats in length, approximately 65 GAA repeats in length, approximately 66 GAA repeats in length, approximately 67 GAA repeats in length, approximately 68 GAA repeats in length, approximately 69 GAA repeats in length, or approximately 70 GAA repeats in length). Thus, provided herein are pegRNAs, complexes, polynucleotides, vectors, cells, compositions, and kits useful in treating Huntington's disease and Friedreich's ataxia, and methods of using the same.

In one aspect, the present disclosure provides pegRNAs for contracting or replacing trinucleotide repeat sequences in the HTT gene. In some embodiments, the present disclosure provides pegRNAs comprising a spacer sequence comprising the nucleic acid sequence: GACCCTGGAAAAGCTGATGA (SEQ ID NO: 381); GCTGCTGCTGGAAGGACTTG (SEQ ID NO: 382); GCTGCTGCTGCTGCTGCTGGA (SEQ ID NO: 383); GCTGCTGCTGCTGCTGCTGC (SEQ ID NO: 384); GGCGGCGGCGGCGGCGGTGG (SEQ ID NO: 385); TGAGGAAGCTGAGGAGGCGG (SEQ ID NO: 386); or GGCGGCTGAGGAAGCTGAGG (SEQ ID NO: 387). In certain embodiments, the pegRNA comprises the sequence of any one of SEQ ID NOs: 454-815, or a sequence at least 80%, at least 85%, at least 90%, at least 95%, at least 96%, at least 97%, at least 98%, or at least 99% identical to the sequence of any one of SEQ ID NOs: 454-815.

In another aspect, the present disclosure provides pegRNAs for deleting trinucleotide repeats sequences in the FXN gene. In some embodiments, the present disclosure provides pegRNAs comprising a spacer sequence comprising the nucleic acid sequence: GCAAGACTAACCTGGCCAACA (SEQ ID NO: 388); GTCCGGAGTTCAAGACTAACC (SEQ ID NO: 389); GAAGGTGGATCACCTGAGGTC (SEQ ID NO: 390); GTCTGGAGTAGCTGGGATTAC (SEQ ID NO: 391); or GCAGGCGCGCGACACCACGCC (SEQ ID NO: 392). In certain embodiments, the pegRNA comprises the sequence of any one of SEQ ID NOs: 816-867, or a sequence at least 80%, at least 85%, at least 90%, at least 95%, at least 96%, at least 97%, at least 98%, or at least 99% identical to the sequence of any one of SEQ ID NOs: 816-867.

In another aspect, the present disclosure provides compositions comprising a prime editor and any of the pegRNAs disclosed herein. Such compositions may be useful, for example, for editing the HTT and FXN genes, and/or for contracting or deleting pathogenic trinucleotide repeat sequences from the HTT and FXN genes. In some embodiments, a composition further comprises any of the nicking guide RNAs (ngRNAs) disclosed herein.

In another aspect, the present disclosure provides methods of treating Huntington's disease by prime editing comprising contacting a target nucleotide sequence with any of the prime editor-pegRNA complexes or compositions provided herein. In some embodiments, the contacting is performed in a cell (e.g., a cell in vitro, or a cell in a human). In certain embodiments, the cell is a cell of the central nervous system (e.g., a neuron).

In another aspect, the present disclosure provides methods of treating Friedreich's ataxia by prime editing comprising contacting a target nucleotide sequence with any of the prime editor-pegRNA complexes or compositions provided herein. In some embodiments, the contacting is performed in a cell (e.g., a cell in vitro, or a cell in a human).

In another aspect, the present disclosure provides polynucleotides encoding any of the pegRNAs and/or prime editor-pegRNA complexes or compositions provided herein. In some embodiments, the present disclosure provides one or more polynucleotides encoding the pegRNA and the prime editor of any of the complexes provided herein.

In another aspect, the present disclosure provides vectors comprising any of the pegRNA—and/or prime editor-encoding polynucleotides provided herein.

In another aspect, the present disclosure provides adeno-associated virus (AAV) particles comprising any of the polynucleotides or vectors provided herein.

In another aspect, the present disclosure provides pharmaceutical compositions comprising any of the pegRNAs, compositions, polynucleotides, vectors, and/or AAV particles provided herein.

In another aspect, the present disclosure provides cells comprising any of the pegRNAs, compositions, polynucleotides, vectors, and/or AAV particles provided herein.

In another aspect, the present disclosure provides kits comprising any of the pegRNAs, compositions, polynucleotides, vectors, and/or AAV particles provided herein.

In another aspect, the present disclosure provides uses of any of the pegRNAs, compositions, polynucleotides, vectors, AAV particles, and/or pharmaceutical compositions provided herein in the treatment of Huntington's disease (including, for example, adult-onset Huntington's disease or juvenile Huntington's disease) or Friedreich's ataxia.

In another aspect, the present disclosure provides uses of any of the pegRNAs, compositions, polynucleotides, vectors, AAV particles, and/or pharmaceutical compositions provided herein in the manufacture of a medicament for the treatment of Huntington's disease or Friedreich's ataxia.

In another aspect, the present disclosure provides methods of using the prime editors, compositions, polynucleotides, or vectors provided herein in veterinary uses. In another aspect, the present disclosure provides methods of using the prime editors, compositions, polynucleotides, or vectors provided herein in agricultural uses.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein.

FIG. 1 shows the rationale for reducing CAG expansions in HTT with prime editing. Huntington's disease (HD) patients carry >35 CAG repeats.¹⁴ Excision of CAG repeats alleviates HD pathology in cell and animal models of HD. This strategy is more precise and safer than other known nuclease-mediated genome editing approaches. SEQ ID NOs: 877, 878 and 879 (top-bottom) are shown.

FIG. 2 provides a schematic of prime editing strategies to reduce CAG repeats. Optimization of the pegRNA spacer is shown. Protospacer sequences upstream and downstream of the CAG tract, PBS lengths of 8-16 nucleotides, and RT templates comprising a CAG insertion+16-40 nucleotides of homology were tested. Spacer 1 showed the highest editing efficiency above background.

FIGS. 3A-3B show optimization of the nicking guide for PE3. PegRNA conditions tested include: a protospacer upstream of the CAG tract; an arbitrary PBS length; and RT templates comprising 26 nt of exon1 of HTT+a 4X CAG insertion+an arbitrary homology length. From all tested nicking conditions, nick N5 resulted in the highest editing efficiency and best editing:indel ratio.

FIGS. 4A-4B show optimization of the PBS and RTT in the pegRNA. PegRNA conditions tested include: the spacer identified with the highest editing efficiency in FIG. 2; a silent PAM mutation; PE3 with nicking gRNA N5 identified in FIG. 3A; PBS lengths of 8-14 nucleotides; and RT templates comprising a 4X CAG insertion+16-35 nucleotides homology. From all conditions tested, a PBS of >8 nucleotides and an RT template of at least 25 nucleotides provided the highest editing efficiency.

FIGS. 5A-5B show testing of various pegRNA structural motifs. PegRNA conditions tested include: the spacer identified with the highest editing efficiency in FIG. 2; a silent PAM mutation; PE3 with nicking gRNA N5 identified in FIG. 3A; a PBS length of 10 nucleotides; an RT template comprising a 4X CAG insertion+31 nucleotides homology; and evopreq1 and mpknot 3′ pegRNA motifs. Of all conditions tested, pegRNAs comprising evopreq1 with a rationally designed linker worked best.

FIG. 6 shows testing of various pegRNA RT template lengths. PegRNA conditions tested include: the spacer identified with the highest editing efficiency in FIG. 2; a silent PAM mutation; PE3 with nicking gRNA N5 identified in FIG. 3A; a PBS length of 10 nucleotides; RT templates comprising a 4X CAG insertion+25-40 nucleotides homology; and an evopreq1 motif. Of all conditions tested, pegRNAs comprising RT templates of 31 nucleotides and 40 nucleotides long provided the best editing efficiencies and the best editing: indel ratios.

FIG. 7 shows a comparison of editing efficiencies between prime editors with and without PEmax architecture. PegRNA conditions tested include: the spacer identified with the highest editing efficiency in FIG. 2; a silent PAM mutation; PE3 with nicking gRNA N5 identified in FIG. 3A; a PBS length of 10 nucleotides; RT templates comprising a 4X CAG insertion+31 or 40 nucleotides homology; and an evopreq1 motif. PEmax outperformed PE2 for epegRNAs with homology of 31 and 40 nucleotides.

FIG. 8 shows the effect of MLH1dn (PE4 and PE5) on HTT editing. PegRNA conditions tested include those identified in FIG. 7. MLH1dn overexpression was observed to improve PE2 editing efficiency.

FIG. 9 shows HTT editing using dual pegRNAs (i.e., twin prime editing). PegRNA conditions tested include those identified in FIG. 7. Single flap prime editing was found to outperform twin prime editing.

FIGS. 10A-10B show testing increasing sizes of the CAG insertion. PegRNA conditions tested include: the spacer identified with the highest editing efficiency in FIG. 2; a silent PAM mutation; PE3 with nicking gRNA N5 identified in FIG. 3A; a PBS length of 10 nucleotides; RT templates comprising 2-9X CAG insertions+31 nucleotides homology; and an evopreq1 motif. Replacement with a smaller number of CAG repeats performed best. The use of a mixture of CAG and CAA repeats did not improve editing efficiency. In FIG. 10A, SEQ ID NOs: 880 (top) and 881 (bottom) are shown.

FIG. 11 shows testing of additional nicking guide options. PegRNA conditions tested include those identified in FIG. 7, with various nicking guide RNAs. Nicking guide RNA N5 was still observed to yield the highest editing efficiency. Nicking guide RNA 23NGA3b was observed to yield the best editing: indel ratio, with high editing efficiency.

FIGS. 12A-12B show improvement of HTT editing through pegRNA scaffold modifications. PegRNA conditions tested include: the spacer identified with the highest editing efficiency in FIG. 2; a silent PAM mutation; a PBS length of 10 nucleotides; RT templates comprising 4X or 9X CAG insertions+31-40 nucleotides homology; and an evopreq1 motif. PegRNAs comprising the U-A flip shown in FIG. 12A showed slightly improved editing efficiency. In FIG. 12A, SEQ ID NOs: 882 (top) and 883 (bottom) are shown.

FIGS. 13A-13B show screening of prime editors with various reverse transcriptase variants for 4X CAG replacement. PegRNA conditions tested include: the spacer identified with the highest editing efficiency in FIG. 2; a silent PAM mutation; PE3 with nicking gRNA N5 identified in FIG. 3A; a PBS length of 10 nucleotides; an RT template comprising a 4X CAG insertion+40 nucleotides homology; and an evopreq1 motif. The prime editor comprising the V223Y MMLV reverse transcriptase variant (PEmax Rhdelta with a V223Y substitution) and pRT-5.800max outperformed PEmax in a PE3 system with the N5 nicking gRNA. Both editors are smaller than PEmax.

FIG. 14 shows screening of prime editors with various reverse transcriptase variants for 9X CAG replacement. PegRNA conditions tested include: the spacer identified with the highest editing efficiency in FIG. 2; a silent PAM mutation; PE3 with nicking gRNA N5 identified in FIG. 3A; a PBS length of 10 nucleotides; an RT template comprising a 9X CAG insertion+40 nucleotides homology; and an evopreq1 motif. The prime editor comprising the V223Y MMLV reverse transcriptase variant (PEmax Rhdelta with a V223Y substitution) and pRT-5.800max outperformed PEmax in a PE3 system with the N5 nicking gRNA. V223Y resulted in lower indels.

FIGS. 15A-15B show improvement of HTT editing by rational design of pegRNA sequences. PegRNA conditions tested include: the spacer identified with the highest editing efficiency in FIG. 2; a silent PAM mutation; PE3 with nicking gRNA N5 identified in FIG. 3A; a PBS length of 10 nucleotides; RT templates comprising a 9X CAG insertion+31-40 nucleotides homology; and an evopreq1 motif. PegRNA with silent mutations in the synthesis template of the RT template sequence generally improve editing efficiency.

FIGS. 16A-16C show improvement of HTT editing by modifying the CAG insertion sequence. In FIG. 16A, SEQ ID NOs: 884 (nt-top), 885 (aa-middle) and 886 (nt-bottom) are shown. In FIG. 16B, SEQ ID NOs: 887-891 (top-bottom) are shown.

FIGS. 17A-17B show further improvement of HTT editing by rational design of pegRNA sequences. PegRNA conditions tested include those used in FIGS. 15A-15B. PegRNA with silent mutations in the synthesis template of the RT template sequence generally improve editing efficiency. In FIG. 17A, SEQ ID NOs: 892 (nt-top), 893 (aa-middle) and 894 (nt-bottom) are shown.

FIGS. 18A-18B show an optimized pegRNA strategy for 6Q (six repeats of the sequence CAG) replacement. Optimized pegRNAs include: the spacer identified with the highest editing efficiency in FIG. 2; a silent PAM mutation; PE3 with nicking gRNA N5 identified in FIG. 3A; a PBS length of 10 nucleotides; an RT template comprising a 9X CAG insertion+31 nucleotides homology; and an evopreq1 motif. In FIG. 18A, SEQ ID NOs: 892 (nt-top), 893 (aa-middle) and 894 (nt-bottom) are shown.

FIGS. 19A-19B show an optimized pegRNA strategy for 11Q replacement. Optimized pegRNAs include: the spacer identified with the highest editing efficiency in FIG. 2; a silent PAM mutation; PE3 with nicking gRNA N5 identified in FIG. 3A; a PBS length of 10 nucleotides; an RT template comprising a 9X CAG insertion+31 or 40 nucleotides homology; and an evopreq1 motif. In FIG. 19A, SEQ ID NOs: 892 (nt-top), 893 (aa-middle) and 894 (nt-bottom) are shown.

FIGS. 20A-20C show optimized conditions for using prime editing to reduce CAG repeats. Optimal pegRNA designs include: 1) a protospacer upstream of the CAG tract; 2) a PBS of 10 nucleotides in length; 3) an RT comprising a 26-nucleotide constant region with a PAM edit and silent mutations, CAG insertions of 4X CAG or 4CAG+3CAA, and 31 or 40 nucleotides of homology; 4) a tevopreq1 motif; and 5) a U-A flip in the pegRNA scaffold as shown in FIG. 12A. Optimal PE2max editor designs include a truncated MMLV (RNAseHdelta) with a V223Y mutation. Optimal PE3 system designs include the N5 nicking guide RNA and PE3b editor with an edit-specific nicking guide (NGA PAM). In FIG. 20A, SEQ ID NOs: 895-899 (top-bottom) are shown.

FIG. 21 shows additional screens of PE variants using the PE3 4CAG strategy. PE variant V223Y outperformed all other variants in the PE3 system with the N5 nicking guide for 4CAG replacement.

FIG. 22 shows additional screens of PE variants using the PE3 4CAG strategy with limited PE to enhance differences. PE variant V223Y with eeCas9-8 outperformed PE variant V223Y and all other variants in the PE3 system with the N5 nicking guide for 4CAG replacement, in the context of limited PE plasmid.

FIG. 23 shows additional screens of PE variants using the PE3 9CAG strategy. PE variant V223Y outperformed all other variants in the PE3 system with the N5 nicking guide for 9CAG replacement.

FIG. 24 shows additional screens of PE variants using the PE3 9CAG strategy with limited PE to enhance differences. PE variant V223Y with eeCas9-8 outperformed PE variant V223Y and all other variants in the PE3 system with the N5 nicking guide for 9CAG replacement, in the context of limited PE plasmid.

FIG. 25 shows additional screens of PE variants using the PE3b 4CAG strategy. PE variant V223Y eeCas9-8 outperformed all other variants in the PE3b system with 23b nicking guide for 4CAG replacement.

FIG. 26 shows additional screens of PE variants using the PE3b 4CAG strategy with limited PE to enhance differences. PE variant V223Y with eeCas9-8 outperformed PE variant V223Y and all other variants in the PE3b system with the 23b nicking guide for 4CAG replacement, in the context of limited PE plasmid.

FIG. 27 shows additional screens of PE variants using the PE3b 9CAG strategy. PE variant V223Y eeCas9-8 outperformed all other variants in the PE3b system with the 23b nicking guide for 9CAG replacement.

FIG. 28 shows additional screens of PE variants using the PE3b 9CAG strategy with limited PE to enhance differences. PE variant V223Y with eeCas9-8 or eeCas9-1 outperformed PE variant V223Y and all other variants in the PE3b system with the 23b nicking guide for 9CAG replacement, in the context of limited PE plasmid.

FIGS. 29A-29B show a second-generation PE strategy to reduce CAG repeats. PegRNA design includes: a protospacer upstream of the CAG tract; a PBS of 10 nucleotides in length; an RT template comprising a 26 nucleotide constant region with PAM edit and silent mutations, a CAG insertion of 4CAG or 9CAR (4CAG+3CAA), and 31 nucleotides or 40 nucleotides homology; a tevopreq1 motif; and a UA flip in the pegRNA scaffold. PE2max editor is used in the PE3b system with the N5 nicking gRNA and NGA PAM. Overall, PE V223Y with eeCas9-1 or eeCas9-8 outperforms PE comprising only the MMLV reverse transcriptase V223Y variant. In FIG. 29A, SEQ ID NOs: 895-897 (top-bottom) are shown. In FIG. 29B, SEQ ID NOs: 898-899 (top-bottom) are shown.

FIG. 30 shows the construct of HTT mESC cells with 21 or 72 CAG repeats. SEQ ID NOs: 900-902 (top-bottom) are shown.

FIGS. 31A-31B show prime editing of the CAG expansion in HTT mESC cells. Conditions used include PE2max, h31p10 eq1 or h40p10 eq1 pegRNA, and N5 nicking guide RNA.

FIGS. 32A-32B show prime editing of the CAG expansion in HTT mESC cells. FIG. 32A shows that pegRNA with homology of 40 nucleotides show better editing efficiency in HTT mESC cells. An extra nicking guide RNA did not improve editing efficiency.

Additionally, editing of longer alleles is more efficient. FIG. 32B shows that in contrast to HEK293T cells with only 17 CAGs or mESC with 21 CAGs, a replacement of a long CAG tract (mESC with 72 CAGs) is nearly as efficient as a replacement with a short repeat sequence.

FIG. 33 provides a schematic for editing of CAG repeats in vivo.

FIGS. 34A-34B show improvement of prime editing of CAG repeats in vivo from the first strategy developed (htt-v1) to the second strategy developed (htt-v2). Compared to htt-v1, the htt-v2 strategy employs PE V223Y with a further optimized pegRNA, an additional nicking guide (PE3), and a stronger promoter in the AAV architecture.

FIGS. 35A-35C show prime editing of CAG repeats in vivo using the htt-v2 strategy. The htt-v2 strategy yielded some editing activity in the brain and liver.

FIGS. 36A-36C show prime editing of CAG repeats in vivo using longer treatment time and improved transduction efficiency. A treatment length of 8 weeks was used. The htt-v2 strategy yielded good transduction efficiency.

FIG. 37 provides a schematic for a third-generation strategy for prime editing of CAG repeats in vivo that employs the use of the PE3b system (htt-v3).

FIGS. 38A-38C show analysis of impure editing results during prime editing of CAG repeats in vivo. Undesired edits are not abundant in HEK293T or HeLa cells but occur at higher frequency in HD patient-derived fibroblasts with long CAG repeats, suggesting that long, endogenous CAGs may cause suboptimal editing outcomes. The htt-v3 strategy utilizing the PE3b system reduces this undesired editing. In FIG. 38A, SEQ ID NOs: 926-927 (top-bottom) are shown.

FIGS. 39A-39D show comparison of the htt-v3 strategy to the htt-v2 strategy. The htt-v3 strategy yielded better editing efficiency and distribution of correct edits.

FIGS. 40A-40B show testing of additional prime editor variants to develop a further improved strategy for prime editing of CAG repeats in vivo (htt-v4). Htt-4a=truncated MMLV reverse transcriptase V223Y+eeCas9-1. Htt-4b=truncated MMLV reverse transcriptase V223Y+eeCas9-8.

FIG. 41 shows the rationale for removal of GAA expansions in FXN with prime editing. Friedreich's ataxia (FRDA or FA) patients carry >65 GAA repeats. Long GAA repeats reduce frataxin mRNA and protein. Excision of GAA repeats alleviates FRDA pathology in cell and animal models. Existing strategies result in a large (>1 kb) deletion. The approach of using prime editing is more precise and safer than other known nuclease-mediated genome editing approaches. SEQ ID NOs: 903 (top) and 904 (bottom) are shown.

FIG. 42 shows optimization of the spacer sequence for removal of GAA repeats by prime editing. PegRNA conditions tested include: protospacers upstream and downstream of the GAA tract; PBS lengths of 8-14 nucleotides; and RT template lengths of 8-40 nucleotides. The pegRNAs were used for deletion of the GAA region and flanking sequence. Of all tested spacer conditions, the spacer sequence “spacer_forw1” resulted in the highest editing efficiency and minimal deletion of the surrounding sequence (45 bp). The longer the RT template, the higher the editing efficiency. SEQ ID NOs: 905-907 (top-bottom) are shown.

FIGS. 43A-43B show optimization of the nicking guide for PE3. PegRNA conditions tested include: the spacer forward 1 sequence identified in FIG. 42; arbitrary PBS lengths; and RT templates comprising arbitrary lengths of homology. Of all tested nicking gRNAs, “nick C” resulted in the highest editing efficiency (equivalent to PE2). In FIG. 43A, SEQ ID NOs: 905, 908-909 (top-bottom) are shown.

FIGS. 44A-44B show optimization of the PBS and RT template in the pegRNA for FXN editing. PegRNA conditions tested include: the spacer forward 1 sequence identified in FIG. 42; PE2 or PE3 with nick C; PBS lengths of 8-14 nucleotides; and RT template lengths of 32-40 nucleotides. Of all conditions tested, an RT template of 40 nucleotides paired with a PBS of 10 nucleotides resulted in the highest editing efficiency. In FIG. 43A, SEQ ID NOS: 910 (top) and 911 (bottom) are shown.

FIGS. 45A-45B show optimization of pegRNA structural motifs for FXN editing. PegRNA conditions tested include: the spacer forward 1 sequence identified in FIG. 42; PE2 or PE3 with nick C; a PBS length of 10 nucleotides; an RT template length of 40 nucleotides; and an evopreq1 motif. A 3′ evopreq1 motif improved editing efficiency in both PE2 and PE3 systems. Use of a linker generated by pegLIT resulted in slightly higher editing efficiency compared to a rationally designed linker.

FIG. 46 shows a comparison of prime editing FXN with and without PEmax architecture. PegRNA conditions tested include those identified in FIGS. 45A-45B, with a pegLIT-designed linker joining the evopreq1 motif to the pegRNA. Prime editors utilizing the PEmax architecture outperformed prime editing using both the PE2 and PE3 systems.

FIG. 47 shows the effect of MLH1dn (PE4 and PE5) on FXN editing. PegRNA conditions tested include those utilized in FIG. 46. MLH1dn overexpression slightly improved PE2 editing efficiency.

FIG. 48 shows FXN editing using dual pegRNAs (twin prime editing). PegRNA conditions tested include those utilized in FIG. 46. Single flap editing outperformed twin prime editing.

FIGS. 49A-49B shows a comparison of PE3 and PE3b for FXN editing. PegRNA conditions tested include those utilized in FIG. 46. Use of PE3b resulted in higher editing efficiency than PE3.

FIGS. 50A-50B show improvement of FXN editing using pegRNA scaffold modifications. PegRNA conditions tested include: the spacer forward 1 sequence identified in FIG. 42; PE2 or PE3b with nick 3b; a PBS length of 10 nucleotides; an RT template length of 40 nucleotides; and an evopreq1 motif with a pegLIT-designed linker. The U-A flip in the pegRNA scaffold, as shown in FIG. 50A, improves editing efficiency in both the PE2 and PE3b systems. In FIG. 50A, SEQ ID NOs: 882 (top) and 883 (bottom) are shown.

FIGS. 51A-51B show screening of prime editors with reverse transcriptase variants. Prime editor conditions tested include those used in FIGS. 50A-50B, including the U-A flip in the pegRNA scaffold as shown in FIG. 50A. MMLV reverse transcriptase comprising a V223Y mutation (PEmax Rhdelta with V223Y) and pRT-5.800max outperform PEmax in the PE3b system with a 3b nicking guide. Both editors are smaller than PEmax.

FIGS. 52A-52B show optimized conditions for using prime editing to excise GAA repeats. Optimal pegRNA conditions include: 1) a protospacer upstream of the GAA tract; 2) a PBS of 10 nucleotides in length; 3) an RT template comprising a 40-nucleotide region of homology; 4) a tevopreq1 motif; and 5) a U-A flip in the pegRNA scaffold as shown in FIG. 50A. Optimal conditions for the prime editor include use of a PE2max editor comprising truncated MMLV (RNAseHdelta) with a V223Y mutation and the typical Cas9 nickase used in PE2max, eeCas9-1, or eeCas9-8, or a PE3b editor with an edit-specific nicking guide. In FIG. 52B, SEQ ID NOs: 912 (top) and 913 (bottom) are shown.

FIG. 53 shows construction of FXN mESC cells with 30 GAA repeats. SEQ ID NOs: 914, 915, 916, 915, and 917 (top-bottom) are shown.

FIG. 54 shows prime editing of the GAA expansion in FXN mESC cells. Conditions tested include the use of PE2max, epegRNA h40p10, and nick C and nick 3b.

FIG. 55 shows results of prime editing of the GAA expansion in FXN mESC cells. pegRNAs with homology of 40 nucleotides worked well in FXN mESC cells, and extra nicking did not improve editing efficiency. Additionally, editing of longer alleles (30 GAAs) was more efficient than editing in HEK293T cells (9 GAAs). PEmax V223Y Rhdelta performs better than or similar to PE2max and has the advantage of being of a smaller size.

FIG. 56 shows screening of prime editors comprising Cas9 variants for FXN editing. The editing strategy includes the following: 1) the protospacer of spacer 1 from FIG. 42; 2) PE3b prime editor with nicking guide RNA; 3) a PBS of 10 nucleotides; 4) a reverse transcriptase template of 40 nucleotides; 5) an evopreq1 motif and pegLIT linker in the pegRNA; 6) a UA scaffold flip in the pegRNA; and 7) prime editor architecture of PE2max comprising truncated MMLV (RNaseHdelta) with a V223Y mutation.

FIG. 57 shows construction of FXN mESCs with 30, 60, and 200 GAA repeats.

FIGS. 58A-58D show prime editing of GAA repeats in vitro.

FIG. 59 shows prime editing of the GAA expansion in FXN mESC cells. The following conditions were used: 1) Protospacer of spacer 1 from FIG. 42; 2) PE2 or PE3b with nicking guide RNA; 3) PBS of 10 nucleotides; 4) Reverse transcriptase template of 40 nucleotides; 5) pegRNA with evopreq1 motif and pegLIT linker; 6) pegRNA with U-A scaffold flip; and 7) PE2max with RNaseH-truncated MMLV reverse transcriptase comprising a V223Y mutation. In FIG. 58A, SEQ ID NOs: 914-917 (top-bottom) are shown.

FIGS. 60A-60B show prime editing of GAA repeats in FRDA fibroblasts. FRDA patient-derived fibroblasts: Control (9 and 9 GAAs); 3816 (330 and 380 GAAs); 4078 (541 and 420 GAAs).

FIGS. 61A-61C show further data for prime editing of GAA repeats in FRDA fibroblasts. FIG. 61A shows Cas9 nuclease editing of GAA repeats. FIG. 61B shows prime editing of GAA repeats in FRDA fibroblasts. FIG. 61C shows FXN expression in FRDA fibroblasts.

FIGS. 62A-62B show delivery approach for prime editing of GAA repeats in vivo. AAV9 was used to deliver an editing system with the following components: 1) Protospacer of spacer 1 in FIG. 42; 2) PE2 prime editor; 3) PBS of 10 nucleotides; 4) Reverse transcriptase template of 40 nucleotides; 5) pegRNA with evopreq1 motif and pegLIT linker; 6) PEmax architecture; and 7) EFS promoter.

FIGS. 63A-63C show optimization of strategy for prime editing of GAA repeats in vivo. AAV9 was used to deliver an editing system with the following components: 1) Protospacer of spacer 1 in FIG. 42; 2) PE3b prime editor with a nicking guide RNA; 3) PBS of 10 nucleotides; 4) Reverse transcriptase template of 40 nucleotides; 5) pegRNA with an evopreq1 motif and pegLIT linker; 6) PEmax architecture with RNaseH-truncated MMLV reverse transcriptase comprising a V223Y mutation; and 7) Cbh promoter.

FIGS. 64A-64C show further data for optimization of prime editing of GAA repeats in vivo. FIG. 64A provides a schematic for ICV injection of mice for prime editing of GAA repeats. FIG. 64B shows FXN prime editing in YG8 mice via injection of AAV9 delivery system. FIG. 64C shows AAV9 transduction efficiency in the cortex of YG8 mice.

FIGS. 65A-65C show FXN expression in prime edited YG8 mice. FIG. 65A shows FXN prime editing in the liver of YG8s mice. FIG. 65B shows FXN expression in the liver of YG8.GAA300 mice. FIG. 65C shows FXN expression in the liver of YG8.GAA800 mice.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

### Adeno-Associated Virus (AAV)

An “adeno-associated virus” or “AAV” is a virus that infects humans and some other primate species. The wild-type AAV genome is a single-stranded deoxyribonucleic acid (ssDNA), either positive- or negative-sensed. The genome comprises two inverted terminal repeats (ITRs), one at each end of the DNA strand, and two open reading frames (ORFs): rep and cap between the ITRs. The rep ORF comprises four overlapping genes encoding Rep proteins required for the AAV life cycle. The cap ORF comprises overlapping genes encoding capsid proteins: VP1, VP2, and VP3, which interact together to form the viral capsid. VP1, VP2, and VP3 are translated from one mRNA transcript, which can be spliced in two different manners: either a longer or shorter intron can be excised resulting in the formation of two isoforms of mRNAs: a ~2.3 kb- and a ~2.6 kb-long mRNA isoform. The capsid forms a supramolecular assembly of approximately 60 individual capsid protein subunits into a non-enveloped, T-1 icosahedral lattice capable of protecting the AAV genome. The mature capsid is composed of VP1, VP2, and VP3 (molecular masses of approximately 87, 73, and 62 kDa, respectively) in a ratio of about 1:1:10.

Recombinant AAV (rAAV) particles may comprise a nucleic acid vector (e.g., a recombinant genome), which may comprise at a minimum: (a) one or more heterologous nucleic acid regions comprising a sequence encoding a protein or polypeptide of interest (e.g., a split prime editor) or an RNA of interest (e.g., a gRNA), or one or more nucleic acid regions comprising a sequence encoding a Rep protein; and (b) one or more regions comprising inverted terminal repeat (ITR) sequences (e.g., wild-type ITR sequences or engineered ITR sequences) flanking the one or more nucleic acid regions (e.g., heterologous nucleic acid regions). In some embodiments, the nucleic acid vector is between 4 kb and 5 kb in size (e.g., 4.2 to 4.7 kb in size). In some embodiments, the nucleic acid vector further comprises a region encoding a Rep protein. In some embodiments, the nucleic acid vector is circular. In some embodiments, the nucleic acid vector is single-stranded. In some embodiments, the nucleic acid vector is double-stranded. In some embodiments, a double-stranded nucleic acid vector may be, for example, a self-complimentary vector that contains a region of the nucleic acid vector that is complementary to another region of the nucleic acid vector, initiating the formation of the double-stranded nucleic acid vector.

### Cas9

The term “Cas9” or “Cas9 nuclease” refers to an RNA-guided nuclease comprising a Cas9 domain, or a fragment thereof (e.g., a protein comprising an active or inactive DNA cleavage domain of Cas9, and/or the gRNA binding domain of Cas9). A “Cas9 domain,” as used herein, is a protein fragment comprising an active or fully or partly inactive cleavage domain of Cas9 and/or the gRNA binding domain of Cas9. A “Cas9 protein” is a full length Cas9 protein. A Cas9 nuclease is also referred to sometimes as a casn1 nuclease or a CRISPR (Clustered Regularly Interspaced Short Palindromic Repeat)-associated nuclease. CRISPR is an adaptive immune system that provides protection against mobile genetic elements (viruses, transposable elements, and conjugative plasmids). CRISPR clusters contain spacers, sequences complementary to antecedent mobile elements, and target invading nucleic acids. CRISPR clusters are transcribed and processed into CRISPR RNA (crRNA). In type II CRISPR systems, correct processing of pre-crRNA requires a trans-encoded small RNA (tracrRNA), endogenous ribonuclease 3 (rnc), and a Cas9 domain. The tracrRNA serves as a guide for ribonuclease 3-aided processing of pre-crRNA. Subsequently, Cas9/crRNA/tracrRNA endonucleolytically cleaves a linear or circular dsDNA target complementary to the spacer. The strand in the target DNA not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gRNA”) can be engineered so as to incorporate aspects of both the crRNA and tracrRNA into a single RNA species. See, e.g., Jinek M., Chylinski K., Fonfara I., Hauer M., Doudna J. A., Charpentier E. Science 337:816-821 (2012), the contents of which are incorporated herein by reference. Cas9 recognizes a short motif in the CRISPR repeat sequences (the PAM or protospacer adjacent motif) to help distinguish self versus non-self. Cas9 nuclease sequences and structures are well known to those of skill in the art (see, e.g., “Complete genome sequence of an M1 strain of Streptococcus pyogenes.” Ferretti et al., J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roe B. A., McLaughlin R. E., Proc. Natl. Acad. Sci. U.S.A. 98:4658-4663 (2001); “CRISPR RNA maturation by trans-encoded small RNA and host factor RNase III.” Deltcheva E., Chylinski K., Sharma C. M., Gonzales K., Chao Y., Pirzada Z. A., Eckert M. R., Vogel J., Charpentier E., Nature 471:602-607 (2011); and “A programmable dual-RNA-guided DNA endonuclease in adaptive bacterial immunity.” Jinek M., Chylinski K., Fonfara I., Hauer M., Doudna J. A., Charpentier E. Science 337:816-821 (2012), the entire contents of each of which are incorporated herein by reference). Cas9 orthologs have been described in various species, including, but not limited to, S. pyogenes and S. thermophilus. Additional suitable Cas9 nucleases and sequences will be apparent to those of skill in the art based on this disclosure, and such Cas9 nucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737; the entire contents of which are incorporated herein by reference. In some embodiments, a Cas9 nuclease comprises one or more mutations that partially impair or inactivate the DNA cleavage domain.

A nuclease-inactivated Cas9 domain may interchangeably be referred to as a “dCas9” protein (for nuclease-“dead” Cas9). Methods for generating a Cas9 domain (or a fragment thereof) having an inactive DNA cleavage domain are known (see, e.g., Jinek et al., Science. 337:816-821 (2012); Qi et al., “Repurposing CRISPR as an RNA-Guided Platform for Sequence-Specific Control of Gene Expression” (2013) Cell. 28; 152(5): 1173-83, the entire contents of each of which are incorporated herein by reference). For example, the DNA cleavage domain of Cas9 is known to include two subdomains, the HNH nuclease subdomain and the RuvC1 subdomain. The HNH subdomain cleaves the strand complementary to the gRNA, whereas the RuvC1 subdomain cleaves the non-complementary strand. Mutations within these subdomains can silence the nuclease activity of Cas9. For example, the mutations D10A and H840A completely inactivate the nuclease activity of S. pyogenes Cas9 (Jinek et al., Science. 337:816-821(2012); Qi et al., Cell. 28; 152(5): 1173-83 (2013)). In some embodiments, proteins comprising fragments of a Cas9 protein are provided. For example, in some embodiments, a protein comprises one of two Cas9 domains: (1) the gRNA binding domain of Cas9; or (2) the DNA cleavage domain of Cas9. In some embodiments, proteins comprising Cas9, or fragments thereof, are referred to as “Cas9 variants.” A Cas9 variant shares homology to Cas9, or a fragment thereof. For example, a Cas9 variant is at least about 70% identical, at least about 80% identical, at least about 90% identical, at least about 95% identical, at least about 96% identical, at least about 97% identical, at least about 98% identical, at least about 99% identical, at least about 99.5% identical, at least about 99.8% identical, or at least about 99.9% identical to wild type Cas9 (e.g., SpCas9 of SEQ ID NO: 6). In some embodiments, the Cas9 variant may have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more amino acid changes compared to wild type Cas9 (e.g., SpCas9 of SEQ ID NO: 6). In some embodiments, the Cas9 variant comprises a fragment of SEQ ID NO: 6 Cas9 (e.g., a gRNA binding domain or a DNA-cleavage domain), such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the corresponding fragment of wild type Cas9 (e.g., SpCas9 of SEQ ID NO: 6). In some embodiments, the fragment is at least 30%, at least 35%, at least 40%, at least 45%, at least 50%, at least 55%, at least 60%, at least 65%, at least 70%, at least 75%, at least 80%, at least 85%, at least 90%, at least 95% identical, at least 96%, at least 97%, at least 98%, at least 99%, or at least 99.5% of the amino acid length of a corresponding wild type Cas9 (e.g., SpCas9 of SEQ ID NO: 6).

### CRISPR

CRISPR is a family of DNA sequences (i.e., CRISPR clusters) in bacteria and archaea that represent snippets of prior infections by a virus that have invaded the prokaryote. The snippets of DNA are used by the prokaryotic cell to detect and destroy DNA from subsequent attacks by similar viruses and effectively compose, along with an array of CRISPR-associated proteins (including Cas9 and homologs thereof) and CRISPR-associated RNA, a prokaryotic immune defense system. In nature, CRISPR clusters are transcribed and processed into CRISPR RNA (crRNA). In certain types of CRISPR systems (e.g., type II CRISPR systems), correct processing of pre-crRNA requires a trans-encoded small RNA (tracrRNA), endogenous ribonuclease 3 (rnc), and a Cas9 protein. The tracrRNA serves as a guide for ribonuclease 3-aided processing of pre-crRNA. Subsequently, Cas9/crRNA/tracrRNA endonucleolytically cleaves a linear or circular dsDNA target complementary to the RNA. Specifically, the DNA strand in the target that is not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gRNA”) can be engineered so as to incorporate aspects of both the crRNA and tracrRNA into a single RNA species—the guide RNA. See, e.g., Jinek M., Chylinski K., Fonfara I., Hauer M., Doudna J. A., Charpentier E. Science 337:816-821 (2012), the entire contents of which is hereby incorporated by reference. Cas9 recognizes a short motif in the CRISPR repeat sequences (the PAM or protospacer adjacent motif) to help distinguish self versus non-self. CRISPR biology, as well as Cas9 nuclease sequences and structures are well known to those of skill in the art (see, e.g., “Complete genome sequence of an M1 strain of Streptococcus pyogenes.” Ferretti et al., J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roe B. A., Mclaughlin R. E., Proc. Natl. Acad. Sci. U.S.A. 98:4658-4663 (2001); “CRISPR RNA maturation by trans-encoded small RNA and host factor RNase III.” Deltcheva E., Chylinski K., Sharma C. M., Gonzales K., Chao Y., Pirzada Z. A., Eckert M. R., Vogel J., Charpentier E., Nature 471:602-607 (2011); and “A programmable dual-RNA-guided DNA endonuclease in adaptive bacterial immunity.” Jinek M., Chylinski K., Fonfara I., Hauer M., Doudna J. A., Charpentier E. Science 337:816-821 (2012), the entire contents of each of which are incorporated herein by reference). Cas9 orthologs have been described in various species, including, but not limited to, S. pyogenes and S. thermophilus. Additional suitable Cas9 nucleases and sequences will be apparent to those of skill in the art based on this disclosure, and such Cas9 nucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737; the entire contents of which are incorporated herein by reference.

In general, a “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g., tracrRNA or an active partial tracrRNA), a tracr mate sequence (encompassing a “direct repeat” and a tracrRNA-processed partial direct repeat in the context of an endogenous CRISPR system), a guide sequence (also referred to as a “spacer”), or other sequences and transcripts from a CRISPR locus. The tracrRNA of the system is complementary (fully or partially) to the tracr mate sequence present on the guide RNA.

### DNA Synthesis Template (or Reverse Transcriptase Template (RTT))

As used herein, the terms “DNA synthesis template” and “reverse transcriptase template (RTT)” refer to the region or portion of the extension arm of a PERNA that is utilized as a template by a polymerase of a prime editor to encode a 3′ single-strand DNA flap that contains the desired edit and which then, through the mechanism of prime editing, replaces the corresponding endogenous strand of DNA at the target site. The extension arm, including the DNA synthesis template, may be comprised of DNA or RNA. In the case of RNA, the polymerase of the prime editor can be an RNA-dependent DNA polymerase (e.g., a reverse transcriptase). In the case of DNA, the polymerase of the prime editor can be a DNA-dependent DNA polymerase. In various embodiments, the DNA synthesis template may comprise the “edit template” and the “homology arm”, and all or a portion of an optional 5′ end modifier region and/or an optional 3′ end modifier region. Said another way, in the case of a 3′ extension arm, the DNA synthesis template can include the portion of the extension arm that spans from the 5′ end of the primer binding site (PBS) to 3′ end of the gRNA core that may operate as a template for the synthesis of a single-strand of DNA by a polymerase (e.g., a reverse transcriptase). In the case of a 5′ extension arm, the DNA synthesis template can include the portion of the extension arm that spans from the 5′ end of the PEgRNA molecule to the 5′ end of the PBS. Certain embodiments described here refer to a “reverse transcriptase template,” an “RT template,” or an “RTT,” which is also inclusive of the edit template and the homology arm, but wherein the RT edit template reflects the use of a prime editor having a polymerase that is a reverse transcriptase, and wherein the DNA synthesis template reflects more broadly the use of a prime editor having any polymerase. In certain embodiments, an RT template may be used to refer to a template polynucleotide for reverse transcription, e.g., in a prime editing system, complex, or method using a prime editor having a polymerase that is a reverse transcriptase. In some embodiments, a DNA synthesis template may be used to refer to a template polynucleotide for DNA polymerization, e.g., RNA-dependent DNA polymerization or DNA-dependent polymerization, e.g., in a prime editing system, complex, or method using a prime editor having a polymerase that is an RNA-dependent DNA polymerase or a DNA-dependent DNA polymerase. The term “edit template” refers to a portion of the extension arm that encodes the desired edit in the single strand 3′ DNA flap that is synthesized by the polymerase, e.g., a DNA-dependent DNA polymerase or an RNA-dependent DNA polymerase (e.g., a reverse transcriptase).

As used herein, the term “DNA synthesis template” refers to the region or portion of the extension arm of a pegRNA that is utilized as a template strand by a polymerase of a prime editor to encode a 3′ single-strand DNA flap that contains the desired edit and which then, through the mechanism of prime editing, replaces the corresponding endogenous strand of DNA at the target site. The extension arm, including the DNA synthesis template, may be comprised of DNA or RNA. In the case of RNA, the polymerase of the prime editor can be an RNA-dependent DNA polymerase (e.g., a reverse transcriptase). In the case of DNA, the polymerase of the prime editor can be a DNA-dependent DNA polymerase. In various embodiments, the DNA synthesis template comprises an the “edit template” and a “homology arm.” In various embodiments, the DNA synthesis template may comprise the “edit template” and a “homology arm”, and all or a portion of the optional 5′ end modifier region, e2. That is, depending on the nature of the e2 region (e.g., whether it includes a hairpin, toeloop, or stem/loop secondary structure), the polymerase may encode none, some, or all of the e2 region, as well. Said another way, in the case of a 3′ extension arm, the DNA synthesis template can include the portion of the extension arm that spans from the 5′ end of the primer binding site (PBS) to 3′ end of the gRNA core that may operate as a template for the synthesis of a single-strand of DNA by a polymerase (e.g., a reverse transcriptase). In the case of a 5′ extension arm, the DNA synthesis template can include the portion of the extension arm that spans from the 5′ end of the pegRNA molecule to the 3′ end of the edit template. In some embodiments, the DNA synthesis template excludes the primer binding site (PBS) of pegRNAs either having a 3′ extension arm or a 5′ extension arm. Certain embodiments refer to an “RT template,” which is inclusive of the edit template and the homology arm, i.e., the sequence of the pegRNA extension arm which is actually used as a template during DNA synthesis. The term “RT template” is equivalent to the term “DNA synthesis template.” In certain embodiments, an RT template may be used to refer to a template polynucleotide for reverse transcription, e.g., in a prime editing system, complex or method using a prime editor having a polymerase that is a reverse transcriptase. In some embodiments, a DNA synthesis template may be used to refer to a template polynucleotide for DNA polymerization, e.g., RNA-dependent DNA polymerization or DNA-dependent polymerization, e.g., in a prime editing system, complex, or method using a prime editor having a polymerase that is an RNA-dependent DNA polymerase or a DNA-dependent DNA polymerase.

In some embodiments, the DNA synthesis template is a single-stranded portion of the PEgRNA that is 5′ of the PBS and comprises a region of complementarity to the PAM strand (i.e., the non-target strand or the edit strand), and comprises one or more nucleotide edits compared to the endogenous sequence of the double stranded target DNA. In some embodiments, the DNA synthesis template is complementary or substantially complementary to a sequence on the non-target strand that is downstream of a nick site, except for one or more non-complementary nucleotides at the intended nucleotide edit positions. In some embodiments, the DNA synthesis template is complementary or substantially complementary to a sequence on the non-target strand that is immediately downstream (i.e., directly downstream) of a nick site, except for one or more non-complementary nucleotides at the intended nucleotide edit positions. In some embodiments, one or more of the non-complementary nucleotides at the intended nucleotide edit positions are immediately downstream of a nick site. In some embodiments, the DNA synthesis template comprises one or more nucleotide edits relative to the double-stranded target DNA sequence. In some embodiments, the DNA synthesis template comprises one or more nucleotide edits relative to the non-target strand of the double-stranded target DNA sequence. For each PEgRNA described herein, a nick site is characteristic of the particular napDNAbp to which the gRNA core of the PERNA associates with, and is characteristic of the particular PAM required for recognition and function of the napDNAbp. For example, for a PEgRNA that comprises a gRNA core that associates with a SpCas9, the nick site in the phosphodiester bond between bases three (“−3” position relative to the position 1 of the PAM sequence) and four (“−4” position relative to position 1 of the PAM sequence). In some embodiments, the DNA synthesis template and the primer binding site are immediately adjacent to each other. The terms “nucleotide edit”, “nucleotide change”, “desired nucleotide change”, and “desired nucleotide edit” are used interchangeably to refer to a specific nucleotide edit, e.g., a specific deletion of one or more nucleotides, a specific insertion of one or more nucleotides, a specific substitution(s) of one or more nucleotides, or a combination thereof, at a specific position in a DNA synthesis template of a PEgRNA to be incorporated in a target DNA sequence. In some embodiments, the DNA synthesis template comprises more than one nucleotide edit relative to the double-stranded target DNA sequence. In such embodiments, each nucleotide edit is a specific nucleotide edit at a specific position in the DNA synthesis template, each nucleotide edit is at a different specific position relative to any of the other nucleotide edits in the DNA synthesis template, and each nucleotide edit is independently selected from a specific deletion of one or more nucleotides, a specific insertion of one or more nucleotides, a specific substitution(s) of one or more nucleotides, or a combination thereof. A nucleotide edit may refer to the edit on the DNA synthesis template as compared to the sequence on the target strand of the double stranded target DNA, or may refer to the edit encoded by the DNA synthesis template on the newly synthesized single stranded DNA that replaces the endogenous target DNA sequence on the non-target strand.

### Edit Strand and Non-Edit Strand

The terms “edit strand” and “non-edit strand” are terms that may be used when describing the mechanism of action of a prime editing system on a double-stranded DNA substrate. The “edit strand” refers to the strand of DNA which is nicked by the prime editor complex to form a 3′ end, which is then extended as a newly synthesized single stranded DNA (also referred herein as the newly synthesized 3′ DNA flap), which comprises a desired edit and ultimately displaces and replaces the single strand region of DNA just downstream of the nick, thereby installing the 3′ DNA flap containing the desired edit downstream of the nick on the “edit strand.” In some embodiments, the newly synthesized 3′ DNA flap comprising the nucleotide edit is paired in a heteroduplex with the non-edit strand that does not comprise the nucleotide edit, thereby creating a mismatch. In some embodiments, the mismatch is recognized by DNA repair machinery, and/or replication machinery, e.g., an endogenous DNA repair machinery. In some embodiments, through DNA repair, the intended nucleotide edit is incorporated into both strands of the target double-stranded DNA substrate. The application may also refer to the “edit strand” as the “protospacer strand” or the “PAM strand” since these elements are present on that strand. The “edit strand” may also be called the “non-target strand” since the edit strand is not the strand that becomes annealed to the spacer of the PERNA molecule, but rather is the complement of the strand that is annealed by the spacer of the PEgRNA. The “non-edit” strand is not directly edited by the PE system. Rather, the desired edit created by the PE system in the 3′ DNA flap is incorporated into the “non-edited strand” through DNA replication and/or repair. In some embodiments, the “non-edit strand” is the strand that anneals to the spacer of the PEgRNA, and thus is also called the “target strand.”

### Extension Arm

The term “extension arm” refers to a nucleotide sequence component of a PEgRNA which comprises a primer binding site (PBS) and a DNA synthesis template for a polymerase (e.g., an RT template for reverse transcriptase). In some embodiments, the extension arm is located at the 3′ end of the guide RNA. In other embodiments, the extension arm is located at the 5′ end of the guide RNA. In some embodiments, the extension arm comprises a DNA synthesis template and a primer binding site. In some embodiments, the extension arm comprises the following components in a 5′ to 3′ direction: the DNA synthesis template, and the primer binding site. In some embodiments, the extension arm also includes a homology arm. In various embodiments, the extension arm comprises the following components in a 5′ to 3′ direction: the homology arm, the edit template, and the primer binding site. Since polymerization activity of the reverse transcriptase is in the 5′ to 3′ direction, the preferred arrangement of the homology arm, edit template, and primer binding site is in the 5′ to 3′ direction such that the reverse transcriptase, once primed by an annealed primer sequence, polymerizes a single strand of DNA using the edit template as a complementary template strand.

The extension arm may be described as comprising generally two regions: a primer binding site (PBS) and a DNA synthesis template, for instance. The primer binding site binds to a primer sequence, for example, a single stranded primer sequence containing a free 3′ end at the nick site that is formed from the endogenous DNA strand of the target site when it becomes nicked by the prime editor complex, thereby exposing a 3′ end on the endogenous nicked strand. As explained herein, the binding of the primer sequence to the primer binding site on the extension arm of the PEgRNA creates a duplex region with an exposed 3′ end (i.e., the 3′ of the primer sequence), which then provides a substrate for a polymerase to begin polymerizing a single strand of DNA from the exposed 3′ end along the length of the DNA synthesis template. The sequence of the single strand DNA product is the complement of the DNA synthesis template. Polymerization continues towards the 5′ of the DNA synthesis template (or extension arm) until polymerization terminates. Thus, the DNA synthesis template represents the portion of the extension arm that is encoded into a single strand DNA product (i.e., the 3′ single strand DNA flap containing the desired nucleotide edit) by the polymerase of the prime editor complex and that ultimately replaces the corresponding endogenous DNA strand of the target site that sits immediately downstream of the PE-induced nick site. Without being bound by theory, polymerization of the DNA synthesis template continues towards the 5′ end of the extension arm until a termination event. Polymerization may terminate in a variety of ways, including, but not limited to (a) reaching a 5′ terminus of the PERNA (e.g., in the case of the 5′ extension arm wherein the DNA polymerase simply runs out of template), (b) reaching an impassable RNA secondary structure (e.g., hairpin or stem/loop), or (c) reaching a replication termination signal, e.g., a specific nucleotide sequence that blocks or inhibits the polymerase, or a nucleic acid topological signal, such as supercoiled DNA or RNA. In some embodiments, a DNA synthesis template (e.g., a reverse transcription template) comprises a nucleotide edit compared to a target HTT gene sequence. In some embodiments, a DNA synthesis template (e.g., a reverse transcription template) comprises or encodes a (CAG) m repeat sequence, wherein m is no more than 35.

### Fusion Protein

The term “fusion protein” as used herein refers to a hybrid polypeptide which comprises protein domains from at least two different proteins. One protein may be located at the amino-terminal (N-terminal) portion of the fusion protein or at the carboxy-terminal (C-terminal) protein thus forming an “amino-terminal fusion protein” or a “carboxy-terminal fusion protein,” respectively. A protein may comprise different domains, for example, a nucleic acid binding domain (e.g., the gRNA binding domain of Cas9 that directs the binding of the protein to a target site) and a nucleic acid cleavage domain or a catalytic domain of a nucleic-acid editing protein. Another example includes fusion of a Cas9 or equivalent thereof to a reverse transcriptase (i.e., a prime editor). Any of the proteins provided herein may be produced by any method known in the art. For example, the proteins provided herein may be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4^{th} ed., Cold Spring Harbor Laboratory Press, Cold Spring Harbor, N.Y. (2012)), the entire contents of which is incorporated herein by reference.

### Genetic Elements

Nucleic acids of the present disclosure (e.g., nucleic acids delivered by an AAV particle as described herein) may include one or more genetic elements. A “genetic element” refers to a particular nucleotide sequence that has a role in nucleic acid expression (e.g., promoter, enhancer, terminator) or encodes a discrete product of an engineered nucleic acid (e.g., a nucleotide sequence encoding a guide RNA and/or a protein).

A “promoter” refers to a control region of a nucleic acid sequence at which initiation and rate of transcription of the remainder of a nucleic acid sequence are controlled. A promoter may also contain sub-regions at which regulatory proteins and molecules may bind, such as an RNA polymerase and other transcription factors. Promoters may be constitutive, inducible, activatable, repressible, tissue-specific, or any combination thereof. A promoter drives expression or drives transcription of the nucleic acid sequence that it regulates. Herein, a promoter is considered to be “operably linked” when it is in a correct functional location and orientation in relation to a nucleic acid sequence it regulates to control (“drive”) transcriptional initiation and/or expression of that sequence.

A promoter may be one naturally associated with a gene or sequence, as may be obtained by isolating the 5′ non-coding sequences located upstream of the coding segment of a given gene or sequence. Such a promoter is referred to as an “endogenous promoter.” In some embodiments, a coding nucleic acid sequence may be positioned under the control of a recombinant or heterologous promoter, which refers to a promoter that is not normally associated with the encoded sequence in its natural environment. Such promoters may include promoters of other genes; promoters isolated from any other cell; and synthetic promoters or enhancers that are not “naturally occurring” such as, for example, those that contain different elements of different transcriptional regulatory regions and/or mutations that alter expression through methods of genetic engineering that are known in the art. In addition to producing nucleic acid sequences of promoters and enhancers synthetically, sequences may be produced using recombinant cloning and/or nucleic acid amplification technology, including polymerase chain reaction (PCR).

In some embodiments, promoters used in accordance with the present disclosure are “inducible promoters,” which are promoters that are characterized by regulating (e.g., initiating or activating) transcriptional activity when in the presence of, influenced by or contacted by an inducer signal. An inducer signal may be endogenous or a normally exogenous condition (e.g., light), compound (e.g., chemical or non-chemical compound), or protein that contacts an inducible promoter in such a way as to be active in regulating transcriptional activity from the inducible promoter. Thus, a “signal that regulates transcription” of a nucleic acid refers to an inducer signal that acts on an inducible promoter. A signal that regulates transcription may activate or inactivate transcription, depending on the regulatory system used. Activation of transcription may involve directly acting on a promoter to drive transcription or indirectly acting on a promoter by inactivation of a repressor that is preventing the promoter from driving transcription. Conversely, deactivation of transcription may involve directly acting on a promoter to prevent transcription or indirectly acting on a promoter by activating a repressor that then acts on the promoter.

A “transcriptional terminator” is a nucleic acid sequence that causes transcription to stop. A transcriptional terminator may be unidirectional or bidirectional. It is comprised of a DNA sequence involved in specific termination of an RNA transcript by an RNA polymerase. A transcriptional terminator sequence prevents transcriptional activation of downstream nucleic acid sequences by upstream promoters. A transcriptional terminator may be necessary in vivo to achieve desirable expression levels or to avoid transcription of certain sequences. A transcriptional terminator is considered to be “operably linked to” a nucleotide sequence when it is able to terminate the transcription of the sequence it is linked to.

The most commonly used type of terminator is a forward terminator. When placed downstream of a nucleic acid sequence that is usually transcribed, a forward transcriptional terminator will cause transcription to abort. In some embodiments, bidirectional transcriptional terminators are provided, which usually cause transcription to terminate on both the forward and reverse strand. In some embodiments, reverse transcriptional terminators are provided, which usually terminate transcription on the reverse strand only.

In prokaryotic systems, terminators usually fall into two categories (1) rho-independent terminators and (2) rho-dependent terminators. Rho-independent terminators are generally composed of a palindromic sequence that forms a stem loop rich in G-C base pairs followed by several T bases. Without wishing to be bound by theory, the conventional model of transcriptional termination is that the stem loop causes RNA polymerase to pause, and transcription of the poly-A tail causes the RNA: DNA duplex to unwind and dissociate from RNA polymerase.

In eukaryotic systems, the terminator region may comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to more stable and are translated more efficiently. Thus, in some embodiments involving eukaryotes, a terminator may comprise a signal for the cleavage of the RNA. In some embodiments, the terminator signal promotes polyadenylation of the message. The terminator and/or polyadenylation site elements may serve to enhance output nucleic acid levels and/or to minimize read through between nucleic acids.

Terminators for use in accordance with the present disclosure include any terminator of transcription described herein or known to one of ordinary skill in the art. Examples of terminators include, without limitation, the termination sequences of genes such as, for example, the bovine growth hormone terminator, and viral termination sequences such as, for example, the SV40 terminator, spy, yejM, secG-leuU, thrLABC, rrnB T1, hisLGDCBHAFI, metZWV, rrnC, xapR, aspA, and arcA terminator. In some embodiments, the termination signal may be a sequence that cannot be transcribed or translated, such as those resulting from a sequence truncation.

Guide RNA (“gRNA”)

As used herein, the term “guide RNA” is a particular type of guide nucleic acid which is mostly commonly associated with a Cas protein of a CRISPR-Cas9 and which associates with Cas9, directing the Cas9 protein to a specific sequence in a DNA molecule that includes complementarity to the spacer sequence of the guide RNA. However, this term also embraces the equivalent guide nucleic acid molecules that associate with Cas9 equivalents, homologs, orthologs, or paralogs, whether naturally occurring or non-naturally occurring (e.g., engineered or recombinant), and which otherwise program the Cas9 equivalent to localize to a specific target nucleotide sequence. The Cas9 equivalents may include other napDNAbp from any type of CRISPR system (e.g., type II, V, VI), including Cpf1 (a type-V CRISPR-Cas systems), C2c1 (a type V CRISPR-Cas system), C2c2 (a type VI CRISPR-Cas system), and C2c3 (a type V CRISPR-Cas system). Further Cas-equivalents are described in Makarova et al., “C2c2 is a single-component programmable RNA-guided RNA-targeting CRISPR effector,” Science 2016; 353 (6299), the contents of which are incorporated herein by reference. Exemplary sequences and structures of guide RNAs are provided herein. In addition, methods for designing appropriate guide RNA sequences are provided herein. As used herein, the “guide RNA” may also be referred to as a “traditional guide RNA” to contrast it with the modified forms of guide RNA termed “prime editing guide RNAs” (or “PEgRNAs”) and “engineered PEgRNAs” (or epegRNAs”).

Guide RNAs or PEgRNAs/epegRNAs may comprise various structural elements that include, but are not limited to:

Spacer sequence—the sequence in the guide RNA or pegRNA/epegRNA (having about 20 nts in length) that has the same sequence as the protospacer in the target DNA, except that the guide RNA or PEgRNA/epegRNA comprises Uracil and the target protospacer contains Thymine.

gRNA core (or gRNA scaffold or backbone sequence)—the sequence within the gRNA that is responsible for binding with a nucleic acid programmable DNA binding protein, e.g., a Cas9. It does not include the spacer sequence that is used to guide Cas9 to target DNA. In some embodiments, a gRNA core sequence (including a gRNA core sequence in a pegRNA) is capable of complexing with a Cas9 protein.

Transcription terminator—the guide RNA or PEgRNA may comprise a transcriptional termination sequence at the 3′ of the molecule.

In some embodiments, a pegRNA or epegRNA may also comprise an extension arm—a single strand extension at the 3′ end or the 5′ end of the PEgRNA which comprises a primer binding site and a DNA synthesis template sequence that encodes via a polymerase (e.g., a reverse transcriptase) a single stranded DNA flap containing the desired nucleotide change, which then integrates into the endogenous DNA by replacing the corresponding endogenous strand, thereby installing the desired nucleotide change.

### Intein

An “intein” is a segment of a protein that is able to excise itself and join the remaining portions (the exteins) with a peptide bond in a process known as protein splicing. Inteins are also referred to as “protein introns.” The process of an intein excising itself and joining the remaining portions of the protein is herein termed “protein splicing” or “intein-mediated protein splicing.” In some embodiments, an intein of a precursor protein (an intein containing protein prior to intein-mediated protein splicing) comes from two genes. Such an intein is referred to herein as a split intein. For example, in cyanobacteria, DnaE, the catalytic subunit a of DNA polymerase III, is encoded by two separate genes, dnaE-n and dnaE-c. The intein encoded by the dnaE-n gene is herein referred as “intein-N.” The intein encoded by the dnaE-c gene is herein referred as “intein-C.”

Other intein systems may also be used. For example, a synthetic intein based on the dnaE intein, the Cfa-N and Cfa-C intein pair, has been described (e.g., in Stevens et al., J. Am. Chem. Soc. 2016 Feb. 24; 138(7): 2162-5, incorporated herein by reference). Non-limiting examples of intein pairs that may be used in accordance with the present disclosure include: Cfa DnaE intein, Ssp GyrB intein, Ssp DnaX intein, Ter DnaE3 intein, Ter ThyX intein, Rma DnaB intein and Cne Prp8 intein (e.g., as described in U.S. Pat. No. 8,394,604, incorporated herein by reference).

Intein-N and intein-C may be fused to the N-terminal portion of the split prime editor and the C-terminal portion of the split prime editor, respectively, for the joining of the N-terminal portion of the split prime editor and the C-terminal portion of the split prime editor. For example, in some embodiments, an intein-N is fused to the C-terminus of the N-terminal portion of the split prime editor, i.e., to form a structure of N—[N-terminal portion of the split prime editor]-[intein-N]—C. In some embodiments, an intein-C is fused to the N-terminus of the C-terminal portion of the split prime editor, i.e., to form a structure of N-[intein-C]—[C-terminal portion of the split prime editor]-C. The mechanism of intein-mediated protein splicing for joining the proteins the inteins are fused to (e.g., split prime editor) is known in the art, e.g., as described in Shah et al., Chem. Sci. 2014; 5(1): 446-461, incorporated herein by reference. In some embodiments, the split site is within a Cas9 protein of a prime editor. In certain embodiments, the split site is between amino acid residues 844 and 845 of a Cas9 protein within a prime editor (e.g., a Cas9 protein of SEQ ID NO: 6). In certain embodiments, the split site is between amino acid residues 1024 and 1025 of a Cas9 protein within a prime editor (e.g., a Cas9 protein of SEQ ID NO: 6).

### Linker

The term “linker,” as used herein, refers to a molecule linking two other molecules or moieties. The linker can be an amino acid sequence in the case of a peptide linker joining two domains of a fusion protein. For example, a napDNAbp (e.g., Cas9) can be fused to a reverse transcriptase by an amino acid linker sequence. The linker can also be a nucleotide sequence in the case of joining two nucleotide sequences together (e.g., in a gRNA). For example, in the instant case, the traditional guide RNA is linked via a spacer or linker nucleotide sequence to the RNA extension of a prime editing guide RNA which may comprise an RT template sequence and an RT primer binding site. In other embodiments, the linker is an organic molecule, group, polymer, or chemical moiety. In some embodiments, the linker is 5-200 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated.

napDNAbp

As used herein, the term “nucleic acid programmable DNA binding protein” or “napDNAbp,” of which Cas9 is an example, refers to a protein that uses RNA: DNA hybridization to target and bind to specific sequences in a DNA molecule. Each napDNAbp is associated with at least one guide nucleic acid (e.g., guide RNA), which localizes the napDNAbp to a DNA sequence that comprises a DNA strand (i.e., a target strand) that is complementary to the guide nucleic acid, or a portion thereof (e.g., the protospacer of a guide RNA). In other words, the guide nucleic-acid “programs” the napDNAbp (e.g., Cas9 or equivalent) to localize and bind to a complementary sequence.

Without being bound by theory, the binding mechanism of a napDNAbp-guide RNA complex, in general, includes the step of forming an R-loop whereby the napDNAbp induces the unwinding of a double-strand DNA target, thereby separating the strands in the region bound by the napDNAbp. The guide RNA protospacer then hybridizes to the “target strand.” This displaces a “non-target strand” that is complementary to the target strand, which forms the single strand region of the R-loop. In some embodiments, the napDNAbp includes one or more nuclease activities, which then cut the DNA, leaving various types of lesions. For example, the napDNAbp may comprise a nuclease activity that cuts the non-target strand at a first location, and/or cuts the target strand at a second location. Depending on the nuclease activity, the target DNA can be cut to form a “double-stranded break” whereby both strands are cut. In other embodiments, the target DNA can be cut at only a single site, i.e., the DNA is “nicked” on one strand. Exemplary napDNAbp with different nuclease activities include “Cas9 nickase” (“nCas9”) and a deactivated Cas9 having no nuclease activities (“dead Cas9” or “dCas9”). Exemplary sequences for these and other napDNAbp are provided herein.

### Nickase

As used herein, a “nickase” refers to a napDNAbp (e.g., a Cas protein) which is capable of cleaving only one of the two complementary strands of a double-stranded target DNA sequence, thereby generating a nick in that strand. In some embodiments, the nickase cleaves a non-target strand of a double stranded target DNA sequence. In some embodiments, the nickase comprises an amino acid sequence with one or more mutations in a catalytic domain of a canonical napDNAbp (e.g., a Cas protein), wherein the one or more mutations reduces or abolishes nuclease activity of the catalytic domain. In some embodiments, the nickase is a Cas9 that comprises one or more mutations in a RuvC-like domain relative to a wild type Cas9 sequence or to an equivalent amino acid position in other Cas9 variants or Cas9 equivalents. In some embodiments, the nickase is a Cas9 that comprises one or more mutations in an HNH-like domain relative to a wild type Cas9 sequence or to an equivalent amino acid position in other Cas9 variants or Cas9 equivalents. In some embodiments, the nickase is a Cas9 that comprises an aspartate-to-alanine substitution (D10A) in the RuvC I catalytic domain of Cas9 relative to a canonical SpCas9 sequence or to an equivalent amino acid position in other Cas9 variants or Cas9 equivalents. In some embodiments, the nickase is a Cas9 that comprises an H840A, N854A, and/or N863A mutation relative to a canonical SpCas9 sequence, or to an equivalent amino acid position in other Cas9 variants or Cas9 equivalents. In some embodiments, the term “Cas9 nickase” refers to a Cas9 with one of the two nuclease domains inactivated. This enzyme is capable of cleaving only one strand of a target DNA. In some embodiments, the nickase is a Cas protein that is not a Cas9 nickase.

In some embodiments, the napDNAbp of the prime editing complex comprises an endonuclease having nucleic acid programmable DNA binding ability. In some embodiments, the napDNAbp comprises an active endonuclease capable of cleaving both strands of a double stranded target DNA. In some embodiments, the napDNAbp is a nuclease active endonuclease, e.g., a nuclease active Cas protein, that can cleave both strands of a double stranded target DNA by generating a nick on each strand. For example, a nuclease active Cas protein can generate a cleavage (a nick) on each strand of a double stranded target DNA. In some embodiments, the two nicks on both strands are staggered nicks, for example, generated by a napDNAbp comprising a Cas12a or Cas12b1. In some embodiments, the two nicks on both strands are at the same genomic position, for example, generated by a napDNAbp comprising a nuclease active Cas9. In some embodiments, the napDNAbp comprises an endonuclease that is a nickase. For example, in some embodiments, the napDNAbp comprises an endonuclease comprising one or more mutations that reduce nuclease activity of the endonuclease, rendering it a nickase. In some embodiments, the napDNAbp comprises an inactive endonuclease, for example, in some embodiments, the napDNAbp comprises an endonuclease comprising one or more mutations that abolish the nuclease activity. In various embodiments, the napDNAbp is a Cas9 protein or variant thereof. The napDNAbp can also be a nuclease active Cas9, a nuclease inactive Cas9 (dCas9), or a Cas9 nickase (nCas9). In a preferred embodiment, the napDNAbp is Cas9 nickase (nCas9) that nicks only a single strand. In other embodiments, the napDNAbp can be selected from the group consisting of: Cas9, Cas12e, Cas12d, Cas12a, Cas12b1, Cas12b2, Cas13a, Cas12c, Cas12d, Cas12e, Cas12h, Cas12i, Cas12g, Cas12f (Cas14), Cas12f1, Cas12j (CasΦ), and Argonaute and optionally has a nickase activity such that only one strand is cut. In some embodiments, the napDNAbp is selected from Cas9, Cas12e, Cas12d, Cas12a, Cas12b1, Cas12b2, Cas13a, Cas12c, Cas12d, Cas12e, Cas12h, Cas12i, Cas12g, Cas12f (Cas14), Cas12f1, Cas12j (Cas), and Argonaute and optionally has a nickase activity such that one DNA strand is cut preferentially to the other DNA strand.

### Nuclear Localization Sequence (NLS)

The term “nuclear localization sequence” or “NLS” refers to an amino acid sequence that promotes import of a protein into the cell nucleus, for example, by nuclear transport. Nuclear localization sequences are known in the art and would be apparent to the skilled artisan. For example, NLS sequences are described in Plank et al., international PCT application, PCT/EP2000/011690, filed Nov. 23, 2000, published as WO/2001/038547 on May 31, 2001, the contents of which are incorporated herein by reference for its disclosure of exemplary nuclear localization sequences. In some embodiments, an NLS comprises the amino acid sequence PKKKRKV (SEQ ID NO: 94).

### Nucleic Acid

The term “nucleic acid,” as used herein, refers to a polymer of nucleotides. The polymer may include natural nucleosides (i.e., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyl adenosine, 5-methylcytidine, C5 bromouridine, C5 fluorouridine, C5 iodouridine, C5 propynyl uridine, C5 propynyl cytidine, C5 methylcytidine, 7-deazaadenosine, 7-deazaguanosine, 8-oxoadenosine, 8-oxoguanosine, O(6)-methylguanine, 4-acetylcytidine, 5-(carboxyhydroxymethyl) uridine, dihydrouridine, methylpseudouridine, 1-methyl adenosine, 1-methyl guanosine, N6-methyl adenosine, and 2-thiocytidine), chemically modified bases, biologically modified bases (e.g., methylated bases), intercalated bases, modified sugars (e.g., 2′-fluororibose, ribose, 2′-deoxyribose, 2′-O-methylcytidine, arabinose, and hexose), or modified phosphate groups (e.g., phosphorothioates and 5′ N phosphoramidite linkages).

### PEgRNA

As used herein, the terms “prime editing guide RNA” or “PERNA” or “pegRNA” or “extended guide RNA” refer to a specialized form of a guide RNA that has been modified to include one or more additional sequences for implementing the prime editing methods and compositions described herein. As described herein, the prime editing guide RNAs comprise one or more “extended regions,” also referred to herein as “extension arms,” of nucleic acid sequence. The extended regions may comprise, but are not limited to, single-stranded RNA or DNA. Further, the extended regions may occur at the 3′ end of a traditional guide RNA. In other arrangements, the extended regions may occur at the 5′ end of a traditional guide RNA. In still other arrangements, the extended region may occur at an intramolecular region of the traditional guide RNA, for example, in the gRNA core region which associates and/or binds to the napDNAbp. The extended region comprises a “DNA synthesis template” which encodes (by the polymerase of the prime editor) a single-stranded DNA which, in turn, has been designed to be (a) homologous with the endogenous target DNA to be edited, and (b) which comprises at least one desired nucleotide change (e.g., a transition, a transversion, a deletion, or an insertion) to be introduced or integrated into the endogenous target DNA. The extended region may also comprise other functional sequence elements, such as, but not limited to, a “primer binding site” and a “linker” sequence, or other structural elements, such as, but not limited to, aptamers, stem loops, hairpins, toe loops (e.g., a 3′ toeloop), or an RNA-protein recruitment domain (e.g., MS2 hairpin). As used herein, the “primer binding site” comprises a sequence that hybridizes to a single-strand DNA sequence having a 3′ end generated from the nicked DNA of the R-loop.

In certain embodiments, the PEgRNAs have a 3′ extension arm, a spacer, and a gRNA core. The 3′ extension arm further comprises in the 5′ to 3′ direction a reverse transcriptase template, a primer binding site, and a linker. The reverse transcriptase template may also be referred to more broadly as the “DNA synthesis template” where the polymerase of a prime editor described herein is not an RT, but another type of polymerase.

In certain other embodiments, the PEgRNAs have a 5′ extension arm, a spacer, and a gRNA core. The 5′ extension further comprises in the 5′ to 3′ direction a reverse transcriptase template, a primer binding site, and a linker. The reverse transcriptase template may also be referred to more broadly as the “DNA synthesis template” where the polymerase of a prime editor described herein is not an RT, but another type of polymerase.

In still other embodiments, the PEgRNAs have in the 5′ to 3′ direction a spacer (1), a gRNA core (2), and an extension arm (3). The extension arm (3) is at the 3′ end of the PEgRNA. The extension arm (3) further comprises in the 5′ to 3′ direction a homology arm, an edit template, and a primer binding site. The extension arm (3) may also comprise an optional modifier region at the 3′ and 5′ ends, which may be the same sequences or different sequences. In addition, the 3′ end of the PEgRNA may comprise a transcriptional terminator sequence. These sequence elements of the PEgRNAs are further described and defined herein.

In still other embodiments, the PEgRNAs have in the 5′ to 3′ direction an extension arm (3), a spacer (1), and a gRNA core (2). The extension arm (3) is at the 5′ end of the PERNA. The extension arm (3) further comprises in the 3′ to 5′ direction a primer binding site, an edit template, and a homology arm. The extension arm (3) may also comprise an optional modifier region at the 3′ and 5′ ends, which may be the same sequences or different sequences. The PEgRNAs may also comprise a transcriptional terminator sequence at the 3′ end. These sequence elements of the PEgRNAs are further described and defined herein.

In some embodiments, the spacer sequence of the pegRNA is about 10, about 11, about 12, about 13, about 14, about 15, about 16, about 17, about 18, about 19, about 20, about 21, about 22, about 23, about 24, or about 25 nucleotides in length. In certain embodiments, the spacer sequence of the pegRNA is about 20 nucleotides in length. In some embodiments, the primer binding site is about 4, about 5, about 6, about 7, about 8, about 9, about 10, about 11, about 12, about 13, about 14, about 15, about 16, or about 17 nucleotides in length. In some embodiments, the homology arm of the pegRNA is about 5, about 6, about 7, about 8, about 9, about 10, about 11, about 12, about 13, about 14, about 15, about 16, about 17, about 18, about 19, or about 20 nucleotides in length. In some embodiments, the DNA synthesis template is from about 5 to about 58 nucleotides in length, about 10 to about 16 nucleotides in length, or about 12 to about 17 nucleotides in length. In certain embodiments, the DNA synthesis template is less than 15 nucleotides in length.

In some embodiments, a pegRNA is an “engineered pegRNA” (“epegRNA”). Relative to a pegRNA, an epegRNA comprises an additional structured motif, for example, attached to its 3′ end. Such additional structured motifs may stabilize the pegRNA or otherwise prevent it from being degraded. Suitable structured motifs include, but are not limited to, toe-loops, hairpins, stem-loops, pseudoknots, aptamers, G-quadruplexes, tRNAs, riboswitches, and ribozymes. In some embodiments, a 3′ structured motif comprises evopreq1.

PegRNAs are further described, e.g., in International Patent Application No. PCT/US2020/023721, filed Mar. 19, 2020, which published as WO 2020/191239; International Patent Application No. PCT/US2021/031439, filed May 7, 2021, which published as WO 2021/226558; International Patent Application No. PCT/2021/052097, filed Sep. 24, 2021, which published as WO 2022/067130; International Patent Application No. PCT/US2022/012054, filed Jan. 11, 2022, which published as WO 2022/150790; International Patent Application No. PCT/US2022/078655, filed Oct. 25, 2022, which published as WO 2023/076898; and International Patent Application No. PCT/US2022/074628, filed Aug. 5, 2022, which published as WO 2023/015309; the contents of each of which is incorporated by reference herein.

### PE1

As used herein, “PE1” refers to a prime editing composition comprising 1) a fusion protein comprising a Cas9 protein variant Cas9 (H840A) and a wild type MMLV RT having the following structure: [NLS]-[Cas9 (H840A)]-[linker]-[MMLV_RT(wt)]-NLS and 2) a desired PEgRNA, wherein the fusion protein (referred to as the PE1 protein) has the amino acid sequence of SEQ ID NO: 3, which is shown as follows.

(SEQ ID NO: 3)
MKRTADGSEFESPKKKRKVDKKYSIGLDIGTNSVGWAVITDEYKVPSKKF
KVLGNTDRHSIKKNLIGALLFDSGETAEATRLKRTARRRYTRRKNRICYL
QEIFSNEMAKVDDSFFHRLEESFLVEEDKKHERHPIFGNIVDEVAYHEKY
PTIYHLRKKLVDSTDKADLRLIYLALAHMIKFRGHFLIEGDLNPDNSDVD
KLFIQLVQTYNQLFEENPINASGVDAKAILSARLSKSRRLENLIAQLPGE
KKNGLFGNLIALSLGLTPNFKSNFDLAEDAKLQLSKDTYDDDLDNLLAQI
GDQYADLFLAAKNLSDAILLSDILRVNTEITKAPLSASMIKRYDEHHQDL
TLLKALVRQQLPEKYKEIFFDQSKNGYAGYIDGGASQEEFYKFIKPILEK
MDGTEELLVKLNREDLLRKQRTFDNGSIPHQIHLGELHAILRRQEDFYPF
LKDNREKIEKILTFRIPYYVGPLARGNSRFAWMTRKSEETITPWNFEEVV
DKGASAQSFIERMTNFDKNLPNEKVLPKHSLLYEYFTVYNELTKVKYVTE
GMRKPAFLSGEQKKAIVDLLFKTNRKVTVKQLKEDYFKKIECFDSVEISG
VEDRFNASLGTYHDLLKIIKDKDFLDNEENEDILEDIVLTLTLFEDREMI
EERLKTYAHLFDDKVMKQLKRRRYTGWGRLSRKLINGIRDKQSGKTILDF
LKSDGFANRNFMQLIHDDSLTFKEDIQKAQVSGQGDSLHEHIANLAGSPA
IKKGILQTVKVVDELVKVMGRHKPENIVIEMARENQTTQKGQKNSRERMK
RIEEGIKELGSQILKEHPVENTQLQNEKLYLYYLQNGRDMYVDQELDINR
LSDYDVDAIVPQSFLKDDSIDNKVLTRSDKNRGKSDNVPSEEVVKKMKNY
WRQLLNAKLITQRKFDNLTKAERGGLSELDKAGFIKRQLVETRQITKHVA
QILDSRMNTKYDENDKLIREVKVITLKSKLVSDFRKDFQFYKVREINNYH
HAHDAYLNAVVGTALIKKYPKLESEFVYGDYKVYDVRKMIAKSEQEIGKA
TAKYFFYSNIMNFFKTEITLANGEIRKRPLIETNGETGEIVWDKGRDFAT
VRKVLSMPQVNIVKKTEVQTGGFSKESILPKRNSDKLIARKKDWDPKKYG
GFDSPTVAYSVLVVAKVEKGKSKKLKSVKELLGITIMERSSFEKNPIDFL
EAKGYKEVKKDLIIKLPKYSLFELENGRKRMLASAGELQKGNELALPSKY
VNFLYLASHYEKLKGSPEDNEQKQLFVEQHKHYLDEIIEQISEFSKRVIL
ADANLDKVLSAYNKHRDKPIREQAENIIHLFTLTNLGAPAAFKYFDTTID
RKRYTSTKEVLDATLIHQSITGLYETRIDLSQLGGDSGGSSGGSSGSETP
GTSESATPESSGGSSGGSSTLNIEDEYRLHETSKEPDVSLGSTWLSDFPQ
AWAETGGMGLAVRQAPLIIPLKATSTPVSIKQYPMSQEARLGIKPHIQRL
LDQGILVPCQSPWNTPLLPVKKPGTNDYRPVQDLREVNKRVEDIHPTVPN
PYNLLSGLPPSHQWYTVLDLKDAFFCLRLHPTSQPLFAFEWRDPEMGISG
QLTWTRLPQGFKNSPTLFDEALHRDLADFRIQHPDLILLQYVDDLLLAAT
SELDCQQGTRALLQTLGNLGYRASAKKAQICQKQVKYLGYLLKEGQRWLT
EARKETVMGQPTPKTPRQLREFLGTAGFCRLWIPGFAEMAAPLYPLTKTG
TLFNWGPDQQKAYQEIKQALLTAPALGLPDLTKPFELFVDEKQGYAKGVL
TQKLGPWRRPVAYLSKKLDPVAAGWPPCLRMVAAIAVLTKDAGKLTMGQP
LVILAPHAVEALVKQPPDRWLSNARMTHYQALLLDTDRVQFGPVVALNPA
TLLPLPEEGLQHNCLDILAEAHGTRPDLTDQPLPDADHTWYTDGSSLLQE
GQRKAGAAVTTETEVIWAKALPAGTSAQRAELIALTQALKMAEGKKLNVY
TDSRYAFATAHIHGEIYRRRGLLTSEGKEIKNKDEILALLKALFLPKRLS
IIHCPGHQKGHSAEARGNRMADQAARKAAITETPDTSTLLIENSSPSGGS
KRTADGSEFEPKKKRKV
KEY:
NUCLEAR LOCALIZATION SEQUENCE (NLS)
TOP: (SEQ ID NO: 95),
BOTTOM: (SEQ ID NO: 96)
CAS9(H840A) (SEQ ID NO: 10)
33-AMINO ACID LINKER (SEQ ID NO: 80)
M-MLV reverse transcriptase (SEQ ID NO: 33)

### PE2

As used herein, “PE2” refers to prime editing composition comprising 1) a fusion protein comprising a Cas9 protein variant Cas9(H840A) and a variant MMLV RT having the following structure: [NLS]-[Cas9(H840A)]-[linker]-[MMLV_RT(D200N)(T330P)(L603W)(T306K)(W313F)]-NLS and 2) a desired PEgRNA, wherein the fusion protein (referred to as the PE2 protein) has the amino acid sequence of SEQ ID NO: 4, which is shown as follows:

(SEQ ID NO: 4)
MKRTADGSEFESPKKKRKVDKKYSIGLDIGTNSVGWAVITDEYKVPSKKF
KVLGNTDRHSIKKNLIGALLFDSGETAEATRLKRTARRRYTRRKNRICYL
QEIFSNEMAKVDDSFFHRLEESFLVEEDKKHERHPIFGNIVDEVAYHEKY
PTIYHLRKKLVDSTDKADLRLIYLALAHMIKFRGHFLIEGDLNPDNSDVD
KLFIQLVQTYNQLFEENPINASGVDAKAILSARLSKSRRLENLIAQLPGE
KKNGLFGNLIALSLGLTPNFKSNFDLAEDAKLQLSKDTYDDDLDNLLAQI
GDQYADLFLAAKNLSDAILLSDILRVNTEITKAPLSASMIKRYDEHHQDL
TLLKALVRQQLPEKYKEIFFDQSKNGYAGYIDGGASQEEFYKFIKPILEK
MDGTEELLVKLNREDLLRKQRTFDNGSIPHQIHLGELHAILRRQEDFYPF
LKDNREKIEKILTFRIPYYVGPLARGNSRFAWMTRKSEETITPWNFEEVV
DKGASAQSFIERMTNFDKNLPNEKVLPKHSLLYEYFTVYNELTKVKYVTE
GMRKPAFLSGEQKKAIVDLLFKTNRKVTVKQLKEDYFKKIECFDSVEISG
VEDRFNASLGTYHDLLKIIKDKDFLDNEENEDILEDIVLTLTLFEDREMI
EERLKTYAHLFDDKVMKQLKRRRYTGWGRLSRKLINGIRDKQSGKTILDF
LKSDGFANRNFMQLIHDDSLTFKEDIQKAQVSGQGDSLHEHIANLAGSPA
IKKGILQTVKVVDELVKVMGRHKPENIVIEMARENQTTQKGQKNSRERMK
RIEEGIKELGSQILKEHPVENTQLQNEKLYLYYLQNGRDMYVDQELDINR
LSDYDVDAIVPQSFLKDDSIDNKVLTRSDKNRGKSDNVPSEEVVKKMKNY
WRQLLNAKLITQRKFDNLTKAERGGLSELDKAGFIKRQLVETRQITKHVA
QILDSRMNTKYDENDKLIREVKVITLKSKLVSDFRKDFQFYKVREINNYH
HAHDAYLNAVVGTALIKKYPKLESEFVYGDYKVYDVRKMIAKSEQEIGKA
TAKYFFYSNIMNFFKTEITLANGEIRKRPLIETNGETGEIVWDKGRDFAT
VRKVLSMPQVNIVKKTEVQTGGFSKESILPKRNSDKLIARKKDWDPKKYG
GFDSPTVAYSVLVVAKVEKGKSKKLKSVKELLGITIMERSSFEKNPIDFL
EAKGYKEVKKDLIIKLPKYSLFELENGRKRMLASAGELQKGNELALPSKY
VNFLYLASHYEKLKGSPEDNEQKQLFVEQHKHYLDEIIEQISEFSKRVIL
ADANLDKVLSAYNKHRDKPIREQAENIIHLFTLTNLGAPAAFKYFDTTID
RKRYTSTKEVLDATLIHQSITGLYETRIDLSQLGGDSGGSSGGSSGSETP
GTSESATPESSGGSSGGSSTLNIEDEYRLHETSKEPDVSLGSTWLSDFPQ
AWAETGGMGLAVRQAPLIIPLKATSTPVSIKQYPMSQEARLGIKPHIQRL
LDQGILVPCQSPWNTPLLPVKKPGTNDYRPVQDLREVNKRVEDIHPTVPN
PYNLLSGLPPSHQWYTVLDLKDAFFCLRLHPTSQPLFAFEWRDPEMGISG
QLTWTRLPQGFKNSPTLFNEALHRDLADFRIQHPDLILLQYVDDLLLAAT
SELDCQQGTRALLQTLGNLGYRASAKKAQICQKQVKYLGYLLKEGQRWLT
EARKETVMGQPTPKTPRQLREFLGKAGFCRLFIPGFAEMAAPLYPLTKPG
TLFNWGPDQQKAYQEIKQALLTAPALGLPDLTKPFELFVDEKQGYAKGVL
TQKLGPWRRPVAYLSKKLDPVAAGWPPCLRMVAAIAVLTKDAGKLTMGQP
LVILAPHAVEALVKQPPDRWLSNARMTHYQALLLDTDRVQFGPVVALNPA
TLLPLPEEGLQHNCLDILAEAHGTRPDLTDQPLPDADHTWYTDGSSLLQE
GQRKAGAAVTTETEVIWAKALPAGTSAQRAELIALTQALKMAEGKKLNVY
TDSRYAFATAHIHGEIYRRRGWLTSEGKEIKNKDEILALLKALFLPKRLS
IIHCPGHQKGHSAEARGNRMADQAARKAAITETPDTSTLLIENSSPSGGS
KRTADGSEFEPKKKRKV
KEY:
NUCLEAR LOCALIZATION SEQUENCE (NLS) TOP:
(SEQ ID NO: 95),
BOTTOM: (SEQ ID NO: 96)
CAS9(H840A) (SEQ ID NO: 10)
33-AMINO ACID LINKER (SEQ ID NO: 80)
M-MLV reverse transcriptase (SEQ ID NO: 34).

### PE3

As used herein, “PE3” refers a prime editing composition comprising a PE2 and further comprising a second-strand nicking guide RNA that complexes with the PE2 and introduces a nick in the non-edit DNA strand in order to induce preferential replacement of the edit strand.

### PE3b

As used herein, “PE3b” refers to a prime editing composition comprising PE2 and further comprising a second-strand nicking guide RNA that complexes with PE2 and introduces a nick in the non-edit DNA strand, wherein the second-strand nicking guide RNA is designed for temporal control such that the second strand nick is not introduced until after the installation of the desired edit. This is achieved by designing the second strand nicking guide RNA with a spacer sequence that comprises complementarity to, and only hybridizes with, only the edited strand after installation of the desired nucleotide edit(s), but not the endogenous target DNA sequence. Using this strategy, mismatches between the nicking guide RNA spacer and the unedited target DNA should disfavor nicking by the sgRNA until after the editing event on the PAM strand takes place.

### PE4

As used herein, “PE4” refers to a prime editing composition comprising a PE2 and further comprising an MLH1 dominant negative protein variant (i.e., wild-type MLH1 with amino acids 754-756 truncated, which may be referred to herein as “MLH1 Δ754-756” or “MLH1dn”). The MLH1 dominant negative protein variant may be expressed in trans in some embodiments. In some embodiments, a PE4 system comprises a fusion protein comprising a PE2 protein and an MLH1 dominant negative protein joined via an optional linker.

### PE5 and PE5b

As used herein, “PE5” refers to a prime editing composition comprising a PE3 and further comprising an MLH1 dominant negative protein variant (i.e., wild-type MLH1 with amino acids 754-756 truncated, which may be referred to as “MLH1 Δ754-756” or “MLH1dn”). The MLH1 dominant negative variant may be expressed in trans in some embodiments. In some embodiments, a PE5 system comprises a fusion protein comprising a PE2 protein and an MLH1 dominant negative protein joined via an optional linker. “PE5b” refers to a prime editing composition comprising a PE3 and an MLH1 dominant negative protein, wherein the second-strand nicking guide RNA is designed for temporal control such that the second strand nick is not introduced until after the installation of the desired edit. This is achieved by designing the second strand nicking guide RNA with a spacer sequence that comprise complementarity to, and hybridizes with, only the edited strand after installation of the desired nucleotide edit(s), but not the endogenous target DNA sequence.

### PEmax

As used herein, “PEmax” refers to a prime editing composition comprising 1) a fusion protein comprising a Cas9 protein variant Cas9(R221K N39K H840A) and a variant MMLV RT having the following structure: [bipartite NLS]-[Cas9(R221K)(N394K)(H840A)]-[linker]-[MMLV_RT(D200N)(T330P)(L603W)]-[bipartite NLS]-[NLS] and 2) a desired PEgRNA, wherein the fusion protein (referred to as the PEmax protein) has the amino acid sequence of SEQ ID NO: 5, which is shown as follows:

(SEQ ID NO: 5)

MKRTADGSEFESPKKKRKVDKKYSIGLDIGTNSVGWAVITDEYKVPSKKF
KVLGNTDRHSIKKNLIGALLFDSGETAEATRLKRTARRRYTRRKNRICYL
QEIFSNEMAKVDDSFFHRLEESFLVEEDKKHERHPIFGNIVDEVAYHEKY
PTIYHLRKKLVDSTDKADLRLIYLALAHMIKFRGHFLIEGDLNPDNSDVD
KLFIQLVQTYNQLFEENPINASGVDAKAILSARLSKSRKLENLIAQLPGE
KKNGLFGNLIALSLGLTPNFKSNFDLAEDAKLQLSKDTYDDDLDNLLAQI
GDQYADLFLAAKNLSDAILLSDILRVNTEITKAPLSASMIKRYDEHHQDL
TLLKALVRQQLPEKYKEIFFDQSKNGYAGYIDGGASQEEFYKFIKPILEK
MDGTEELLVKLKREDLLRKQRTFDNGSIPHQIHLGELHAILRRQEDFYPF
LKDNREKIEKILTFRIPYYVGPLARGNSRFAWMTRKSEETITPWNFEEVV
DKGASAQSFIERMTNFDKNLPNEKVLPKHSLLYEYFTVYNELTKVKYVTE
GMRKPAFLSGEQKKAIVDLLFKTNRKVTVKQLKEDYFKKIECFDSVEISG
VEDRFNASLGTYHDLLKIIKDKDFLDNEENEDILEDIVLTLTLFEDREMI
EERLKTYAHLFDDKVMKQLKRRRYTGWGRLSRKLINGIRDKQSGKTILDF
LKSDGFANRNFMQLIHDDSLTFKEDIQKAQVSGQGDSLHEHIANLAGSPA
IKKGILQTVKVVDELVKVMGRHKPENIVIEMARENQTTQKGQKNSRERMK
RIEEGIKELGSQILKEHPVENTQLQNEKLYLYYLQNGRDMYVDQELDINR
LSDYDVDAIVPQSFLKDDSIDNKVLTRSDKNRGKSDNVPSEEVVKKMKNY
WRQLLNAKLITQRKFDNLTKAERGGLSELDKAGFIKRQLVETRQITKHVA
QILDSRMNTKYDENDKLIREVKVITLKSKLVSDFRKDFQFYKVREINNYH
HAHDAYLNAVVGTALIKKYPKLESEFVYGDYKVYDVRKMIAKSEQEIGKA
TAKYFFYSNIMNFFKTEITLANGEIRKRPLIETNGETGEIVWDKGRDFAT
VRKVLSMPQVNIVKKTEVQTGGFSKESILPKRNSDKLIARKKDWDPKKYG
GFDSPTVAYSVLVVAKVEKGKSKKLKSVKELLGITIMERSSFEKNPIDFL
EAKGYKEVKKDLIIKLPKYSLFELENGRKRMLASAGELQKGNELALPSKY
VNFLYLASHYEKLKGSPEDNEQKQLFVEQHKHYLDEIIEQISEFSKRVIL
ADANLDKVLSAYNKHRDKPIREQAENIIHLFTLTNLGAPAAFKYFDTTID
RKRYTSTKEVLDATLIHQSITGLYETRIDLSQLGGDSGGSSGGSKRTADG
SEFESPKKKRKVSGGSSGGSTLNIEDEYRLHETSKEPDVSLGSTWLSDFP
QAWAETGGMGLAVRQAPLIIPLKATSTPVSIKQYPMSQEARLGIKPHIQR
LLDQGILVPCQSPWNTPLLPVKKPGTNDYRPVQDLREVNKRVEDIHPTVP
NPYNLLSGLPPSHQWYTVLDLKDAFFCLRLHPTSQPLFAFEWRDPEMGIS
GQLTWTRLPQGFKNSPTLFNEALHRDLADFRIQHPDLILLQYVDDLLLAA
TSELDCQQGTRALLQTLGNLGYRASAKKAQICQKQVKYLGYLLKEGQRWL
TEARKETVMGQPTPKTPRQLREFLGKAGFCRLFIPGFAEMAAPLYPLTKP
GTLFNWGPDQQKAYQEIKQALLTAPALGLPDLTKPFELFVDEKQGYAKGV
LTQKLGPWRRPVAYLSKKLDPVAAGWPPCLRMVAAIAVLTKDAGKLTMGQ
PLVILAPHAVEALVKQPPDRWLSNARMTHYQALLLDTDRVQFGPVVALNP
ATLLPLPEEGLQHNCLDILAEAHGTRPDLTDQPLPDADHTWYTDGSSLLQ
EGQRKAGAAVTTETEVIWAKALPAGTSAQRAELIALTQALKMAEGKKLNV
YTDSRYAFATAHIHGEIYRRRGWLTSEGKEIKNKDEILALLKALFLPKRL

SIIHCPGHQKGHSAEARGNRMADQAARKAAITETPDTSTLLIENSSPSGG
SKRTADGSEFESPKKKRKVGSGPAAKRVKLD
KEY:
BIPARTITE SV40 NUCLEAR LOCALIZATION SEQUENCE (NLS)
TOP: (SEQ ID NO: 95),
CAS9(R221K N39K H840A) (SEQ ID NO: 2)
SGGSx2-BIPARTITE SV40NLS-SGGSx2 LINKER
(SEQ ID NO: 79)
M-MLV reverse transcriptase
(D200N T306K W313F T330P L603W)
(SEQ ID NO: 34)
Other linker sequence (SEQ ID NOs: 82)
BIPARTITE SV40NLS (SEQ ID NO: 97)
Other linker sequence
c-Myc NLS (SEQ ID NO: 98)

### PE3max and PE3bmax

As used herein, “PE3max” refers to a prime editing composition comprising a PEmax protein, a desired pegRNA, and a second strand nicking guide RNA. In some embodiments, PE3max can be considered as PE3 except wherein the PE2 component is substituted with PEmax. “PE3bmax” refers to a prime editing composition comprising a PEmax protein, a desired pegRNA, and a second strand nicking guide RNA, wherein the second-strand nicking guide RNA is designed for temporal control such that the second strand nick is not introduced until after the installation of the desired edit. This is achieved by designing the second strand nicking guide RNA with a spacer sequence that comprise complementarity to, and hybridizes with, only the edited strand after installation of the desired nucleotide edit(s), but not the endogenous target DNA sequence.

### PE4max

As used herein, “PE4max” refers to PE4 but the PE2 component is substituted with PEmax.

### PE5max and PE5bmax

As used herein, “PE5max” refers to PE5, but the PE2 component of PE3 is substituted with PEmax. “PE5bmax” refers to PE5b, but the PE2 component of PE3 is substituted with PEmax.

### PE6

The term “PE6” refers to a suite of next-generation prime editors described herein (PE6a, PE6b, PE6c, PE6d, PE6e, PE6f, and PE6g) comprising improved reverse transcriptase and/or Cas9 variants. The improved reverse transcriptase and Cas9 domains of the PE6 variants can also be combined with each other to offer cumulative benefits. For example, a PE6 prime editor comprising an improved reverse transcriptase variant of PE6a and an improved Cas9 variant of PE6e is referred to herein as the prime editor “PE6a-e” (or “PE6e-a”). Any possible combination of PE6 prime editors is contemplated by the present disclosure including, for example, PE6a-e, PE6a-f, PE6a-g, PE6b-e, PE6b-f, PE6b-g, PE6c-e, PE6c-f, PE6c-g, PE6d-e, PE6d-f, and PE6d-g.

PE6a comprises a reverse transcriptase variant comprising the amino acid substitutions E60K, K87E, E165D, D243N, R267I, E279K, K318E, and K343N relative to an Ec48 reverse transcriptase (SEQ ID NO: 59). PE6b comprises a reverse transcriptase variant comprising the amino acid substitutions P70T, G72V, S87G, M102I, K106R, K118R, I128V, L158Q, F269L, A363V, K413E, and S492N relative to a Tf1 reverse transcriptase (SEQ ID NO: 55). PE6c comprises a reverse transcriptase variant comprising the amino acid substitutions P70T, G72V, S87G, M102I, K106R, K118R, I128V, L158Q, S188K, I260L, F269L, R288Q, S297Q, A363V, K413E, and S492N relative to a Tf1 reverse transcriptase (SEQ ID NO: 55). PE6d comprises a reverse transcriptase variant comprising the amino acid substitutions T128N, D200C, and V223Y (and the substitutions T306K, W313F, and T330P used in the MMLV reverse transcriptase of PE2 and PEmax) relative to a MMLV reverse transcriptase (SEQ ID NO: 33) with a truncation of the C-terminal RnaseH domain (e.g., between D497 and I498 of SEQ ID NO: 33). PE6e comprises a Cas9 variant comprising the amino acid substitutions K775R and K918A relative to wild type Streptococcus pyogenes Cas9 or Streptococcus pyogenes Cas9 nickase (SEQ ID NO: 7). PE6f comprises a Cas9 variant comprising the amino acid substitutions H99R, E471K, 1632V, D645N, H721Y, and K918A relative to wild type Streptococcus pyogenes Cas9 or Streptococcus pyogenes Cas9 nickase (SEQ ID NO: 7). PE6g comprises a Cas9 variant comprising the amino acid substitutions H99R, E471K, 1632V, D645N, R654C, and H721Y relative to wild type Streptococcus pyogenes Cas9 or Streptococcus pyogenes Cas9 nickase (SEQ ID NO: 7). Any of the PE6 prime editors provided herein may also comprise the architecture of the PEmax protein as provided below. In some embodiments, any of the PE6 prime editors provided herein may further comprise additional amino acid mutations, e.g., any of those included in PEmax as provided below.

### PE7

The term “PE7” refers to the PE6 prime editors plus a second strand nicking guide RNA. For example, “PE7a” refers to the PE6a prime editor as provided herein, plus a second strand nicking guide RNA.

### Polymerase

As used herein, the term “polymerase” refers to an enzyme that synthesizes a nucleotide strand and that may be used in connection with the prime editor delivery systems described herein. The polymerase can be a “template-dependent” polymerase (i.e., a polymerase that synthesizes a nucleotide strand based on the order of nucleotide bases of a template strand). The polymerase can also be a “template-independent” polymerase (i.e., a polymerase that synthesizes a nucleotide strand without the requirement of a template strand). A polymerase may also be further categorized as a “DNA polymerase” or an “RNA polymerase.” In various embodiments, the prime editor system comprises a DNA polymerase. In various embodiments, the DNA polymerase can be a “DNA-dependent DNA polymerase” (i.e., whereby the template molecule is a strand of DNA). In such cases, the DNA template molecule can be a PERNA, wherein the extension arm comprises a strand of DNA. In such cases, the PEgRNA may be referred to as a chimeric or hybrid PEgRNA which comprises an RNA portion (i.e., the guide RNA components, including the spacer and the gRNA core) and a DNA portion (i.e., the extension arm). In various other embodiments, the DNA polymerase can be an “RNA-dependent DNA polymerase” (i.e., whereby the template molecule is a strand of RNA). In such cases, the PEgRNA is RNA, i.e., including an RNA extension. The term “polymerase” may also refer to an enzyme that catalyzes the polymerization of nucleotides (i.e., the polymerase activity). Generally, the enzyme will initiate synthesis at the 3′-end of a primer annealed to a polynucleotide template sequence (e.g., such as a primer sequence annealed to the primer binding site of a PERNA) and will proceed toward the 5′ end of the template strand. A “DNA polymerase” catalyzes the polymerization of deoxynucleotides. As used herein in reference to a DNA polymerase, the term DNA polymerase includes a “functional fragment thereof.” A “functional fragment thereof” refers to any portion of a wild-type or mutant DNA polymerase that encompasses less than the entire amino acid sequence of the polymerase and which retains the ability, under at least one set of conditions, to catalyze the polymerization of a polynucleotide. Such a functional fragment may exist as a separate entity, or it may be a constituent of a larger polypeptide, such as a fusion protein.

### Prime Editing

As used herein, the term “prime editing” refers to an approach for gene editing using napDNAbps, a polymerase (e.g., a reverse transcriptase), and specialized guide RNAs that include a primer binding site and a DNA synthesis template for encoding desired new genetic information (or deleting genetic information) that is then incorporated into a target DNA sequence. Prime editing is described in Anzalone, A. V. et al., Search-and-replace genome editing without double-strand breaks or donor DNA. Nature 576, 149-157 (2019), which is incorporated herein by reference.

Prime editing represents a platform for genome editing that is a versatile and precise method to directly write new genetic information into a specified DNA site using a nucleic acid programmable DNA binding protein (“napDNAbp”) working in association with a polymerase (i.e., in the form of a fusion protein or otherwise provided in trans with the napDNAbp), wherein the prime editing system is programmed with a prime editing (PE) guide RNA (“PEgRNA”) that both specifies the target site and templates the synthesis of the desired edit in the form of a replacement DNA strand by way of an extension (either DNA or RNA) engineered onto a guide RNA (e.g., at the 5′ or 3′ end, or at an internal portion of a guide RNA). The replacement strand containing the desired edit (e.g., a single nucleobase substitution) shares the same sequence as the endogenous strand (or is homologous to it) immediately downstream of the nick site of the target site to be edited (with the exception that it includes the desired edit). Through DNA repair and/or replication machinery, the endogenous strand downstream of the nick site is replaced by the newly synthesized replacement strand containing the desired edit. In some cases, prime editing may be thought of as a “search-and-replace” genome editing technology since the prime editors, as described herein, not only search and locate the desired target site to be edited, but at the same time, encode a replacement strand containing a desired edit that is installed in place of the corresponding target site endogenous DNA strand. The prime editors of the present disclosure relate, in part, to the discovery that the mechanism of target-primed reverse transcription (TPRT) or “prime editing” can be leveraged or adapted for conducting precision CRISPR/Cas-based genome editing with high efficiency and genetic flexibility. TPRT is naturally used by mobile DNA elements, such as mammalian non-LTR retrotransposons and bacterial Group II introns. Cas protein-reverse transcriptase fusions or related systems are used to target a specific DNA sequence with a guide RNA, generate a single strand nick at the target site, and use the nicked DNA as a primer for reverse transcription of an engineered reverse transcriptase template that is integrated with the guide RNA. However, while the concept begins with prime editors that use reverse transcriptase as the DNA polymerase component, the prime editors described herein are not limited to reverse transcriptases but may include the use of virtually any DNA polymerase. Indeed, while the application throughout may refer to prime editors with “reverse transcriptases,” it is set forth here that reverse transcriptases are only one type of DNA polymerase that may work with prime editing. Thus, wherever the specification mentions a “reverse transcriptase,” the person having ordinary skill in the art should appreciate that any suitable DNA polymerase may be used in place of the reverse transcriptase. Thus, in one aspect, the prime editors may comprise Cas9 (or an equivalent napDNAbp), which is programmed to target a DNA sequence by associating it with a specialized guide RNA (i.e., PEgRNA) containing a spacer sequence that anneals to a complementary sequence (the complementary sequence to an endogenous protospacer sequence) in the target DNA. The PEgRNA also contains new genetic information in the form of an extension that encodes a replacement strand of DNA containing a desired nucleotide change which is used to replace a corresponding endogenous DNA strand at the target site. To transfer information from the PEgRNA to the target DNA, the mechanism of prime editing involves nicking the target site in one strand of the DNA to expose a 3′-hydroxyl group. The exposed 3′-hydroxyl group can then be used to prime the DNA polymerization of the edit-encoding extension on PEgRNA directly into the target site. In various embodiments, the extension—which provides the template for polymerization of the replacement strand containing the edit—can be formed from RNA or DNA. In the case of an RNA extension, the polymerase of the prime editor can be an RNA-dependent DNA polymerase (such as a reverse transcriptase). In the case of a DNA extension, the polymerase of the prime editor may be a DNA-dependent DNA polymerase. The newly synthesized strand (i.e., the replacement DNA strand containing the desired nucleotide edit) that is formed by the prime editor would be homologous to the genomic target sequence (i.e., have the same sequence as), except for the inclusion of one or more desired nucleotide changes (e.g., a single nucleotide substitution, a deletion, or an insertion, or a combination thereof). The newly synthesized (or replacement) strand of DNA may also be referred to as a single strand DNA flap, which would compete for hybridization with the complementary homologous endogenous DNA strand, thereby displacing the corresponding endogenous strand. Resolution of the hybridized intermediate (also referred to as a heteroduplex, comprising the single strand DNA flap synthesized by the reverse transcriptase hybridized to the endogenous DNA strand with the exception of mismatches at positions where desired nucleotide edits are installed in the edit strand) can include removal of the resulting displaced flap of endogenous DNA (e.g., with a 5′ end DNA flap endonuclease, FEN1), ligation of the synthesized single strand DNA flap to the target DNA, and assimilation of the desired nucleotide changes as a result of cellular DNA repair and/or replication processes. Because templated DNA synthesis offers single nucleotide precision for the modification of any nucleotide, including insertions and deletions, the scope of this approach is very broad and could foreseeably be used for myriad applications in basic science and therapeutics. In certain embodiments, the system can be combined with the use of an error-prone reverse transcriptase enzyme (e.g., provided as a fusion protein with the Cas9 domain, or provided in trans to the Cas9 domain). The error-prone reverse transcriptase enzyme can introduce alterations during synthesis of the single strand DNA flap. Thus, in certain embodiments, error-prone reverse transcriptase can be utilized to introduce nucleotide changes to the target DNA. Depending on the error-prone reverse transcriptase that is used with the system, the changes can be random or non-random.

In various embodiments, prime editing operates by contacting a target DNA molecule (for which a change in the nucleotide sequence is desired to be introduced) with a nucleic acid programmable DNA binding protein (napDNAbp) complexed with a prime editing guide RNA (PEgRNA). In various embodiments, the prime editing guide RNA (PEgRNA) comprises an extension at the 3′ or 5′ end of the guide RNA, or at an intramolecular location in the guide RNA, and encodes the desired nucleotide change (e.g., single nucleotide substitution, insertion, or deletion). First, the napDNAbp/extended gRNA complex contacts the DNA molecule, and the extended gRNA guides the napDNAbp to bind to a target locus. Next, a nick in one of the strands of DNA of the target locus is introduced (e.g., by a nuclease or chemical agent), thereby creating an available 3′ end in one of the strands of the target locus. In certain embodiments, the nick is created in the strand of DNA that corresponds to the R-loop strand, i.e., the strand that is not hybridized to the guide RNA sequence, i.e., the “non-target strand.” The nick, however, could be introduced in either of the strands. That is, the nick could be introduced into the R-loop “target strand” (i.e., the strand hybridized to the protospacer of the extended gRNA) or the “non-target strand” (i.e., the strand forming the single-stranded portion of the R-loop and which is complementary to the target strand). In the next step, the 3′ end of the DNA strand (formed by the nick) interacts with the extended portion of the guide RNA in order to prime reverse transcription (i.e., “target-primed RT”). In certain embodiments, the 3′ end DNA strand hybridizes to a specific RT priming sequence on the extended portion of the guide RNA, i.e., the “reverse transcriptase priming sequence” or “primer binding site” on the PEgRNA. In the next step, a reverse transcriptase (or other suitable DNA polymerase) is introduced that synthesizes a single strand of DNA from the 3′ end of the primed site towards the 5′ end of the prime editing guide RNA. The DNA polymerase (e.g., reverse transcriptase) can be fused to the napDNAbp or alternatively can be provided in trans to the napDNAbp. This forms a single-strand DNA flap comprising the desired nucleotide change (e.g., the single base change, insertion, or deletion, or a combination thereof) and that is otherwise homologous to the endogenous DNA at or adjacent to the nick site. In the next step, the napDNAbp and guide RNA are released. The final two steps relate to the resolution of the single strand DNA flap such that the desired nucleotide change becomes incorporated into the target locus. This process can be driven towards the desired product formation by removing the corresponding 5′ endogenous DNA flap that forms once the 3′ single strand DNA flap invades and hybridizes to the endogenous DNA sequence. Without being bound by theory, the cell's endogenous DNA repair and replication processes resolve the mismatched DNA to incorporate the nucleotide change(s) to form the desired altered product. The process can also be driven towards product formation with “second strand nicking.” This process may introduce at least one or more of the following genetic changes: transversions, transitions, deletions, and insertions.

The term “prime editor (PE) system” or “prime editor (PE)” or “PE system” or “PE editing system” refers the compositions involved in the method of genome editing using target-primed reverse transcription (TPRT) described herein, including, but not limited to, the napDNAbps, reverse transcriptases, fusion proteins (e.g., comprising napDNAbps and reverse transcriptases), prime editing guide RNAs, and complexes comprising fusion proteins and prime editing guide RNAs, as well as accessory elements, such as second strand nicking components (e.g., second strand nicking sgRNAs) and 5′ endogenous DNA flap removal endonucleases (e.g., FEN1) for helping to drive the prime editing process towards the edited product formation.

Although in the embodiments described thus far the PEgRNA constitutes a single molecule comprising a guide RNA (which itself comprises a spacer sequence and a gRNA core or scaffold) and a 5′ or 3′ extension arm comprising the primer binding site and a DNA synthesis template, the PEgRNA may also take the form of two individual molecules. For example, in some embodiments, a PEgRNA may comprise a guide RNA and a trans prime editor RNA template (tPERT), which essentially houses the extension arm (including, in particular, the primer binding site and the DNA synthesis domain) and an RNA-protein recruitment domain (e.g., MS2 aptamer or hairpin) in the same molecule which becomes co-localized or recruited to a modified prime editor complex that comprises a tPERT recruiting protein (e.g., MS2cp protein, which binds to the MS2 aptamer).

Prime editing, twin prime editing, and prime editors are further described, e.g., in International Patent Application No. PCT/US2020/023721, filed Mar. 19, 2020, which published as WO 2020/191239; International Patent Application No. PCT/US2021/031439, filed May 7, 2021, which published as WO 2021/226558; International Patent Application No. PCT/2021/052097, filed Sep. 24, 2021, which published as WO 2022/067130; International Patent Application No. PCT/US2022/012054, filed Jan. 11, 2022, which published as WO 2022/150790; International Patent Application No. PCT/US2022/078655, filed Oct. 25, 2022, which published as WO 2023/076898; and International Patent Application No. PCT/US2022/074628, filed Aug. 5, 2022, which published as WO 2023/015309; the contents of each of which is incorporated by reference herein.

### Prime Editor

The term “prime editor” refers to the polypeptide or polypeptide components involved in prime editing as described herein. In some embodiments, a prime editor comprises a fusion constructs comprising a napDNAbp (e.g., Cas9 nickase) and a reverse transcriptase. In some embodiments, a prime editor is capable of carrying out prime editing on a target nucleotide sequence in the presence of a PEgRNA (or “extended guide RNA”). In some embodiments, a prime editor comprises a napDNAbp (e.g., Cas9 nickase) and a reverse transcriptase provided in trans, i.e., the napDNAbp and the reverse transcriptase are not fused. The in trans napDNAbp and the reverse transcriptase may be tethered via a non-peptide linkage, e.g., a MS2 RNA-protein binding RNA sequence and a MS2 coat protein fused to either the napDNAbp or the reverse transcriptase, or may be unlinked to each other and simply recruited by the pegRNA. In some embodiments, a prime editor composition, system, or complex provided herein comprises a fusion protein or a fusion protein complexed with a PEgRNA, and/or further complexed with a second-strand nicking sgRNA. In some embodiments, the prime editor system may also refer to the complex comprising a fusion protein (reverse transcriptase fused to a napDNAbp), a PERNA, and a regular guide RNA capable of directing the second-site nicking step of the non-edited strand as described herein.

### Primer Binding Site

The term “primer binding site” or “PBS” refers to the portion of a PEgRNA as a component of the extension arm (e.g., at the 3′ end of the extension arm), and is a single-stranded portion of the PERNA as a component of the extension arm that comprises a region of complementarity to a sequence on the non-target strand of a double stranded target DNA. In some embodiments, the primer binding site is complementary to a region upstream of a nick site in a non-target strand. In some embodiments, the primer binding site is complementary to a region immediately upstream of a nick site in the non-target strand. In some embodiments, the primer binding site is capable of binding to the primer sequence that is formed after nicking of the edit strand (the non-target strand) of the target DNA sequence by the prime editor. When the prime editor (e.g., by a Cas9 nickase component of a prime editor) nicks the edit strand of the target DNA sequence, a free 3′ end is formed in the edit strand, which serves as a primer sequence that anneals to the primer binding site on the PEgRNA to prime reverse transcription. In some embodiments, the PBS is complementary to or substantially complementary to and can anneal to a free 3′ end on the non-target strand of the double stranded target DNA at the nick site. In some embodiments, the PBS anneals to the free 3′ end on the non-target strand can initiate target-primed DNA synthesis. In some embodiments, a PBS comprises complementarity to nucleotides 1 to (n−3) of a spacer sequence.

### Protein, Peptide, and Polypeptide

The terms “protein,” “peptide,” and “polypeptide” are used interchangeably herein and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, recombinant, or synthetic, or any combination thereof. Any of the proteins provided herein may be produced by any method known in the art. For example, the proteins provided herein may be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4th ed., Cold Spring Harbor Laboratory Press, Cold Spring Harbor, N.Y. (2012)), the contents of which are incorporated herein by reference.

### Protospacer

As used herein, the term “protospacer” refers to the sequence (e.g., of ~20 bp) in DNA adjacent to the PAM (protospacer adjacent motif) sequence. The protospacer shares the same sequence as the spacer sequence of the guide RNA (except that a protospacer contains Thymine and the spacer sequence contains Uracil). The guide RNA anneals to the complement of the protospacer sequence on the target DNA (specifically, one strand thereof, i.e., the “target strand” versus the “non-target strand” of the target DNA sequence). In some embodiments, in order for a Cas nickase component of a prime editor to function, it also requires a specific protospacer adjacent motif (PAM) that varies depending on the Cas protein component itself, e.g., the type of Cas protein and the bacterial species from which it is derived. The most commonly used Cas9 nuclease, derived from S. pyogenes, recognizes a PAM sequence of NGG that is directly downstream of the protospacer sequence in the genomic DNA, on the non-target strand.

### Protospacer Adjacent Motif (PAM)

As used herein, the term “protospacer adjacent motif” or “PAM” refers to a DNA sequence (e.g., an approximately 2-6 nucleotide sequence) that is an important targeting component of a Cas nuclease, e.g., a Cas9. For example, in some embodiments for a Cas9 nuclease, the PAM sequence is on either strand and is downstream in the 5′ to 3′ direction of the Cas9 cut site. The canonical PAM sequence (i.e., the PAM sequence that is associated with the Cas9 nuclease of Streptococcus pyogenes or SpCas9) is 5′-NGG-3′, wherein “N” is any nucleobase followed by two guanine (“G”) nucleobases. In some embodiments, SpCas9's can also recognize additional non-canonical PAMs (e.g., NAG and NGA).

Different PAM sequences can be associated with different Cas9 nucleases or equivalent proteins from different organisms. In addition, any given Cas9 nuclease, e.g., SpCas9, may be modified to alter the PAM specificity of the nuclease such that the nuclease recognizes an alternative PAM sequence.

For example, with reference to the canonical SpCas9 amino acid sequence SEQ ID NO: 6, the PAM sequence can be modified by introducing one or more mutations, including (a) D1135V, R1335Q, and T1337R “the VQR variant,” which alters the PAM specificity to NGAN or NGNG, (b) D1135E, R1335Q, and T1337R “the EQR variant,” which alters the PAM specificity to NGAG, and (c) D1135V, G1218R, R1335E, and T1337R “the VRER variant,” which alters the PAM specificity to NGCG. In addition, the D1135E variant of canonical SpCas9 still recognizes NGG, but it is more selective compared to the wild type SpCas9 protein.

It will also be appreciated that Cas9 enzymes from different bacterial species (i.e., Cas9 orthologs) can have varying PAM specificities. For example, Cas9 from Staphylococcus aureus (SaCas9) recognizes NGRRT or NGRRN. In addition, Cas9 from Neisseria meningitis (NmCas) recognizes NNNNGATT. In another example, Cas9 from Streptococcus thermophilis (StCas9) recognizes NNAGAAW. In still another example, Cas9 from Treponema denticola (TdCas) recognizes NAAAAC. These are examples and are not meant to be limiting. It will be further appreciated that non-SpCas9s bind a variety of PAM sequences, which makes them useful when no suitable SpCas9 PAM sequence is present at the desired target cut site. Furthermore, non-SpCas9s may have other characteristics that make them more useful than SpCas9. For example, Cas9 from Staphylococcus aureus (SaCas9) is about 1 kilobase smaller than SpCas9, so it can be packaged into adeno-associated virus (AAV). Further reference is made to Shah et al., “Protospacer recognition motifs: mixed identities and functional diversity,” RNA Biology, 10 (5): 891-899 (which is incorporated herein by reference).

### Reverse Transcriptase

The term “reverse transcriptase” describes a class of polymerases characterized as RNA-dependent DNA polymerases. All known reverse transcriptases require a primer to synthesize a DNA transcript from an RNA template. Historically, reverse transcriptase has been used primarily to transcribe mRNA into cDNA, which can then be cloned into a vector for further manipulation. Avian myoblastosis virus (AMV) reverse transcriptase was the first widely used RNA-dependent DNA polymerase (Verma, Biochim. Biophys. Acta 473:1 (1977)). The enzyme has 5′-3′ RNA-directed DNA polymerase activity, 5′-3′ DNA-directed DNA polymerase activity, and RNase H activity. RNase H is a processive 5′ and 3′ ribonuclease specific for the RNA strand for RNA-DNA hybrids (Perbal, A Practical Guide to Molecular Cloning, New York: Wiley & Sons (1984)). Errors in transcription cannot be corrected by reverse transcriptase because known viral reverse transcriptases lack the 3′-5′ exonuclease activity necessary for proofreading (Saunders and Saunders, Microbial Genetics Applied to Biotechnology, London: Croom Helm (1987)). A detailed study of the activity of AMV reverse transcriptase and its associated RNaseH activity has been presented by Berger et al., Biochemistry 22:2365-2372 (1983). Another reverse transcriptase that is used extensively in molecular biology is reverse transcriptase originating from Moloney murine leukemia virus (M-MLV or “MMLV”). See, e.g., Gerard, G. R., DNA 5:271-279 (1986) and Kotewicz, M. L., et al., Gene 35:249-258 (1985). M-MLV reverse transcriptase substantially lacking in RNase H activity has also been described. See, e.g., U.S. Pat. No. 5,244,797. The invention contemplates the use of any such reverse transcriptases, or variants or mutants thereof.

In addition, the invention contemplates the use of reverse transcriptases that are error-prone, i.e., that may be referred to as error-prone reverse transcriptases or reverse transcriptases that do not support high fidelity incorporation of nucleotides during polymerization. During synthesis of the single-strand DNA flap based on the RT template integrated with the guide RNA, the error-prone reverse transcriptase can introduce one or more nucleotides that are mismatched with the RT template sequence, thereby introducing changes to the nucleotide sequence through erroneous polymerization of the single-strand DNA flap. These errors introduced during synthesis of the single strand DNA flap then become integrated into the double strand molecule through hybridization to the corresponding endogenous target strand, removal of the endogenous displaced strand, ligation, and then through one more round of endogenous DNA repair and/or sequencing processes. In some embodiments, the prime editors used in the complexes and methods provided herein comprise MMLV RT.

### Reverse Transcription

As used herein, the term “reverse transcription” indicates the capability of an enzyme to synthesize a DNA strand (that is, complementary DNA or cDNA) using RNA as a template. In some embodiments, the reverse transcription can be “error-prone reverse transcription,” which refers to the properties of certain reverse transcriptase enzymes that are error-prone in their DNA polymerization activity.

### Second-Strand Nicking

Prime editing typically involves the resolution of heteroduplex DNA (i.e., containing one edited and one non-edited strand) formed as a result of installation of one or more desired nucleotide changes in the edit strand but not (yet) in the non-edit strand of the target DNA sequence. Resolution of the heteroduplex DNA (the edited strand paired with the endogenous non-edited strand) and installation of nucleotide changes corresponding to the desired nucleotide edits in the non-edit strand permanently integrates the desired edits in the target DNA sequence. The approach of “second-strand nicking” can be used herein to help drive the resolution of heteroduplex DNA in favor of permanent integration of the edited strand into the DNA molecule. As used herein, the concept of “second-strand nicking” refers to the introduction of a second nick on the unedited strand. In some embodiments, a second nick is introduced at a location on the non-edit strand corresponding to a position downstream of the first nick (i.e., the initial nick site that provides the free 3′ end for use in priming of the reverse transcriptase on the extended portion of the guide RNA) on the edit strand. Thus, the first nick (introduced by the prime editor in combination with the PEgRNA) and the second nick (introduced by the prime editor and a second-strand nicking guide RNA) are on opposite strands. Said another way, the first nick is on the non-target strand (i.e., the strand that forms the single strand portion of the R-loop), and the second nick is on the target strand. Said still another way, the first nick (introduced by the prime editor in combination with the PERNA) is on the edit strand, and the second nick (introduced by the prime editor and second strand nicking guide RNA) is on the non-edit strand. The second nick can be introduced in the non-edit strand at a position that is opposite at least 1, 2, 3, 4, or 5 nucleotides downstream or upstream of the first nick of the edit strand, or that is opposite at least 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, 110, 120, 130, 140, or 150 or more nucleotides downstream or upstream of the first nick of the edit strand. The second nick can also be introduced in the non-edit strand at a position that is opposite at least 1, 2, 3, 4, or 5 nucleotides downstream or upstream of the edit site of the edit strand, or that is opposite at least 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, 110, 120, 130, 140, or 150 or more nucleotides downstream or upstream of the edit site of the edit strand. The second nick, in certain embodiments, can be introduced in the non-edit strand at a position that is opposite about 1-150 nucleotides downstream or upstream of the first nick of the edit strand, or that is opposite about 1-140, or about 1-130, or about 1-120, or about 1-110, or about 1-100, or about 1-90, or about 1-80, or about 1-70, or about 1-60, or about 1-50, or about 1-40, or about 1-30, or about 1-20, or about 1-10 nucleotides downstream or upstream of the first nick of the edit strand. Without being bound by theory, the second nick induces the cell's endogenous DNA repair and replication processes towards replacement of the non-edit strand, thereby permanently installing the edited sequence on both strands of the target DNA and resolving the heteroduplex that is formed as a result of PE.

In certain embodiments, the second strand nicking guide RNA (also referred to herein as the nicking guide RNA, ngRNA, secondary nicking RNA, or second strand nicking sgRNA) may include a spacer sequence that preferentially and/or selectively only anneals to the edit strand after the desired nucleotide edit(s) are installed but not to the original strand of DNA the becomes replaced by the edited strand (i.e., the 5′ single-strand DNA flap that is displaced and ultimately removed during heteroduplex resolution). This can operate by designing the second strand nicking guide RNA to comprise a spacer sequence that anneals only to the edited region of the edited strand (and thus, wherein the spacer of the second strand nicking guide RNA comprises a nucleotide sequence that is the complement of the edited sequence or region thereof and includes the complement of the edit) and thus, can discriminate between the edited strand and the original strand of the displaced 5′ single-strand DNA flap that is immediately downstream of the cut site of the edited strand. This can be referred to as “temporal second-strand nicking” because the second strand nicking occurs only after prime editing has generated the new 3′ DNA flap containing the desired edit. This avoids the introduction of a double strand cut during prime editing which would otherwise result from the simultaneous or approximately simultaneous cutting of opposite strands by the PE complex comprising the PEgRNA and the PE complex comprising the second-strand cutting guide RNA.

### Spacer Sequence

As used herein, the term “spacer sequence” in connection with a guide RNA or a PEgRNA refers to the portion of the guide RNA or PEgRNA of about 20 nucleotides that contains a nucleotide sequence that shares the same sequence as the protospacer sequence in the target DNA sequence. The spacer sequence anneals to the complement of the protospacer sequence to form a ssRNA/ssDNA hybrid structure at the target site and a corresponding R loop ssDNA structure of the endogenous DNA strand.

### Split Cas9

A “split Cas9” refers to a fusion protein with a split site located within the Cas9 protein that is provided as an N-terminal portion (also referred to as an N-terminal half) and a C-terminal portion (also referred to as a C-terminal half) encoded by two separate nucleotide sequences. The polypeptides corresponding to the N-terminal portion and the C-terminal portion of the Cas9 protein may be combined (joined) to form a complete Cas9 protein. A Cas9 protein is known to consist of a bi-lobed structure linked by a disordered linker (e.g., as described in Nishimasu et al., Cell, Volume 156, Issue 5, pp. 935-949, 2014, incorporated herein by reference). In some embodiments, the “split” occurs between the two lobes, generating two portions of a Cas9 protein, each containing one lobe.

### Split Intein

Although inteins are most frequently found as a contiguous domain, some exist in a naturally split form. In this case, the two fragments are expressed as separate polypeptides and must associate before splicing takes place, so-called protein trans-splicing.

An exemplary split intein is the Ssp DnaE intein, which comprises two subunits, namely, DnaE-N and DnaE-C. The two different subunits are encoded by separate genes, namely dnaE-n and dnaE-c, which encode the DnaE-N and DnaE-C subunits, respectively. DnaE is a naturally occurring split intein in Synechocytis sp. PCC6803 and is capable of directing trans-splicing of two separate proteins, each comprising a fusion with either DnaE-N or DnaE-C.

Additional naturally occurring or engineered split-intein sequences are known in the art or can be made from whole-intein sequences described herein or those available in the art. Examples of split-intein sequences can be found in Stevens et al., “A promiscuous split intein with expanded protein engineering applications,” PNAS, 2017, Vol. 114:8538-8543; Iwai et al., “Highly efficient protein trans-splicing by a naturally split DnaE intein from Nostc punctiforme, FEBS Lett, 580:1853-1858, each of which are incorporated herein by reference. Additional split intein sequences can be found, for example, in WO 2013/045632, WO 2014/055782, WO 2016/069774, and EP2877490, the contents each of which are incorporated herein by reference.

In addition, protein splicing in trans has been described in vivo and in vitro (Shingledecker, et al., Gene 207:187 (1998), Southworth, et al., EMBO J. 17:918 (1998); Mills, et al., Proc. Natl. Acad. Sci. USA, 95:3543-3548 (1998); Lew, et al., J. Biol. Chem., 273:15887-15890 (1998); Wu, et al., Biochim. Biophys. Acta 35732:1 (1998b), Yamazaki, et al., J. Am. Chem. Soc. 120:5591 (1998), Evans, et al., J. Biol. Chem. 275:9091 (2000); Otomo, et al., Biochemistry 38:16040-16044 (1999); Otomo, et al., J. Biolmol. NMR 14:105-114 (1999); Scott, et al., Proc. Natl. Acad. Sci. USA 96:13638-13643 (1999)) and provides the opportunity to express a protein as to two inactive fragments that subsequently undergo ligation to form a functional product.

### Split Prime Editor

A “split prime editor” refers to a prime editor that is provided as an N-terminal portion (also referred to as a N-terminal half) and a C-terminal portion (also referred to as a C-terminal half) encoded by two separate nucleic acids. The polypeptides corresponding to the N-terminal portion and the C-terminal portion of the prime editor may be combined to form a complete prime editor. In some embodiments, for a prime editor that comprises a dCas9 or nCas9, the “split” is located in the dCas9 or nCas9 domain, at positions as described herein in the split prime editor. Accordingly, in some embodiments, the N-terminal portion of the prime editor contains the N-terminal portion of the split prime editor, and the C-terminal portion of the prime editor contains the C-terminal portion of the split prime editor. Similarly, intein-N or intein-C may be fused to the N-terminal portion or the C-terminal portion of the prime editor, respectively, for the joining of the N- and C-terminal portions of the prime editor to form a complete prime editor. In some embodiments, the split site is within a Cas9 protein of a prime editor. In certain embodiments, the split site is between amino acid residues 844 and 845 of a Cas9 protein within a prime editor (e.g., a Cas9 protein of SEQ ID NO: 6). In certain embodiments, the split site is between amino acid residues 1024 and 1025 of a Cas9 protein within a prime editor (e.g., a Cas9 protein of SEQ ID NO: 6).

### Silent Mutation

As used herein, the term “silent mutation” refers to a mutation in a nucleic acid molecule that does not have an effect on the phenotype of the nucleic acid molecule, or the protein it produces if it encodes a protein. Silent mutations can be introduced into coding regions of a nucleic acid (i.e., segments of a gene that encode for a protein), or they can be introduced in non-coding regions of a nucleic acid. A silent mutation in a nucleic acid sequence, e.g., in a target DNA sequence or in a DNA synthesis template sequence to be installed in the target sequence, may be a nucleotide alteration that does not result in expression or function of the amino acid sequence encoded by the nucleic acid sequence, or other functional features of the target nucleic acid sequence. When silent mutations are present in a coding region, they may be synonymous mutations. Synonymous mutations refer to substitutions of one base for another in a gene such that the corresponding amino acid residue of the protein produced by the gene is not modified. This is due to the redundancy of the genetic code, allowing for multiple different codons to encode for the same amino acid in a particular organism. When a silent mutation is in a noncoding region or a junction of a coding region and a non-coding region (e.g., an intron/exon junction), it may be in a region that does not impact any biological properties of the nucleic acid molecule (e.g., splicing, gene regulation, RNA lifetime, etc.). In particular embodiments, a silent mutation may also be a “benign” mutation, for example, where a nucleotide substitution results in one or more alterations in the amino acid sequence encoded, but does not result in detrimental impact on the expression or function of the polypeptide. Silent mutations may be useful, for example, for increasing the length of contiguous changes in a desired nucleotide edit or the number of nucleotide edits made to a target nucleotide sequence using prime editing to evade correction of the edit by the MMR pathway. In certain embodiments, the number of silent mutations installed may be one, or two, or three, or four, or five, or six, or seven, or eight, or nine, or ten, or more. In certain other embodiments involving at least two silent mutations, the silent mutations may be installed within one, or two, or three, or four, or five, or six, or seven, or eight, or nine, or ten, or 11, 12, 13, 14, 15, 16, 17, 18, 19, 20, 21, 22, 23, 24, or 25 nucleotides from the intended edit site. In some embodiments, silent mutations are installed in order to alter or optimize the secondary structure that a particular pegRNA will form in cell. In some embodiments, changing some bases of a pegRNA with silent mutations results in changes to the secondary structure of the pegRNA that can improve editing efficiency.

### Subject

The term “subject,” as used herein, refers to an individual organism, for example, an individual mammal. In some embodiments, the subject is a human. In some embodiments, the subject is a non-human mammal. In some embodiments, the subject is a non-human primate. In some embodiments, the subject is a rodent. In some embodiments, the subject is a sheep, a goat, a cattle, a cat, or a dog. In some embodiments, the subject is a vertebrate, an amphibian, a reptile, a fish, an insect, a fly, or a nematode. In some embodiments, the subject is a research animal. In some embodiments, the subject is genetically engineered, e.g., a genetically engineered non-human subject. The subject may be of either sex and may be at any stage of development. In some embodiments, a subject has or is suspected of having a triplet repeat disorder. In some embodiments, a subject has or is suspected of having Huntington's disease. In some embodiments, a subject has or is suspected of having Friedreich's ataxia.

### Target Site

The term “target site” refers to a sequence within a nucleic acid molecule that is edited by a prime editor (PE) disclosed herein. The target site further refers to the sequence within a nucleic acid molecule to which a complex of the prime editor (PE) and gRNA binds. In some embodiments, a target site comprises a trinucleotide repeat sequence. In some embodiments, a target site comprises part of the HTT gene. In some embodiments, a target site comprises part of the FXN gene.

### Temporal Second-Strand Nicking

As used herein, the term “temporal second-strand nicking” refers to a variant of second strand nicking whereby the installation of the second-strand nick in the unedited strand occurs only after the desired edit is installed in the edited strand by the PE complexed with the PERNA. Without being bound by theory, the second-strand nick in the unedited strand induces the cell's endogenous DNA repair and replication processes towards replacement of the unedited strand, thereby permanently installing the edited sequence on both strands and resolving the heteroduplex that is formed as a result of PE. In some embodiments, a prime editor system comprising a second strand nicking guide RNA designed with the temporal second strand nicking strategy, which can avoid concurrent nicks on both strands that could lead to double-stranded DNA breaks. The second-strand nicking guide RNA is designed for temporal control such that the second strand nick is not introduced until after the installation of the desired edit. This is achieved by designing a gRNA with a spacer sequence that matches only the edited strand, but not the original allele. Using this strategy, mismatches between the spacer of the second-strand nicking guide RNA and the unedited allele should disfavor second-strand nicking until after the editing event on the PAM strand takes place. In certain embodiments, the second strand nicking guide RNA may include a spacer sequence that preferentially and/or selectively only anneals to the edited strand (i.e., after PE synthesizes the edit), but not to the original strand of DNA that becomes replaced by the edited strand (i.e., the 5′ single-strand DNA flap that is displaced and ultimately removed during heteroduplex resolution). This can operate by designing the second strand nicking guide RNA to comprise a spacer sequence that anneals only to the edited region of the edited strand (and thus, wherein the spacer of the second strand nicking guide RNA comprises a nucleotide sequence that is the complement of the edited sequence or region thereof and includes the complement of the edit) and thus, can discriminate between the edited strand and the original strand of the displaced 5′ single-strand DNA flap that is immediately downstream of the cut site of the edited strand. This avoids the introduction of a double strand cut during prime editing that would otherwise result from the simultaneous or approximately simultaneous cutting of opposite strands by the PE complex comprising the PEgRNA and the PE complex comprising the second-strand cutting guide RNA.

In some embodiments, a prime editor system (e.g., a PE3b system or a PE5b system) comprises components that improve temporal second-strand nicking by including PE-based installation of one or more silent mutations around an edit site (e.g., introducing one or more silent mutations located upstream and/or downstream of a non-silent, desired nucleotide edit or adjacent to the non-silent nucleotide edit). In some embodiments, a prime editor system comprises a pegRNA, the DNA synthesis template of which comprises one or more non-silent nucleotide edits and further comprises one or more silent mutations compared to the endogenous sequence of the target strand (and accordingly encodes a single stranded DNA comprising the one or more non-silent nucleotide edits and the silent mutations compared to the endogenous sequence of the edit strand). In some embodiments, the one or more silent mutations are adjacent to or immediately adjacent to a non-silent nucleotide edit in the DNA synthesis template. For example, in some embodiments, the one or more silent mutations are within 5 nucleotides upstream of the non-silent nucleotide edit. In some embodiments, the one or more silent mutations are within 5 nucleotides downstream of the non-silent nucleotide edit. In some embodiments, the one or more silent mutations are immediately adjacent to the non-silent nucleotide edit, such that the DNA synthesis template contains at least 3 contiguous nucleotides that are not complementary to the corresponding endogenous sequence downstream of the nick site on the edit strand of the target DNA sequence. Without wishing to be bound by a particular theory, such silent mutations may improve prime editing efficiency by evading the cellular mismatch repair pathway by avoiding reversion of the PE-installed edit on the edit strand back to the pre-edited sequence. Such silent mutations may also improve prime editing efficiency by altering or optimizing the secondary structure of the pegRNA. In some embodiments, a prime editor system comprising a pegRNA with the one or more silent mutations in addition to the non-silent mutation in the DNA synthesis template can result in improved editing efficiency of the target DNA, as compared to a control prime editor system comprising a pegRNA that only contains the non-silent mutation and not the one or more silent mutations in the DNA synthesis template. In some embodiments, combining PE3b designs with the silent mutations can further improve prime editing efficiency and/or reduce indel frequency resulted from editing. This can operate by designing a second strand nicking guide RNA that comprises a spacer sequence that anneals only to the edited strand, which includes not only a desired edit, but also the one or more installed silent mutations that are installed at proximal continuous or non-continuous positions near the desired edit. The single-strand nicking guide RNA comprises a spacer sequence that is complementary to the PE-edited strand can discriminate between the edited strand and the original strand, which corresponds to the displaced 5′ single-strand DNA flap that is immediately downstream of the first nick site of the edited strand. This improved strategy of temporal second-strand nicking avoids the introduction of a double strand cut during prime editing that would otherwise result from the simultaneous or approximately simultaneous cutting of opposite strands by the PE complex comprising the PEgRNA and the PE complex comprising the second-strand cutting guide RNA.

The silent mutations may be installed in coding regions of the target nucleic acid molecule or in non-coding regions of the target nucleic acid molecule. When the silent mutations are installed in a coding region, they introduce into the nucleic acid molecule one or more alternate codons encoding the same amino acid as the unedited nucleic acid molecule. Alternatively, when the silent mutations are installed in a non-coding region, the silent mutations may be present in a region of the nucleic acid molecule that does not influence splicing, gene regulation, RNA lifetime, or other biological properties of the target site on the nucleic acid molecule.

### Treatment

The terms “treatment,” “treat,” and “treating” refer to a clinical intervention aimed to reverse, alleviate, delay the onset of, or inhibit the progress of a disease or disorder, or one or more symptoms thereof, as described herein. As used herein, the terms “treatment,” “treat,” and “treating” refer to a clinical intervention aimed to reverse, alleviate, delay the onset of, or inhibit the progress of a disease or disorder, or one or more symptoms thereof, as described herein. In some embodiments, treatment may be administered after one or more symptoms have developed and/or after a disease has been diagnosed. In other embodiments, treatment may be administered in the absence of symptoms, e.g., to prevent or delay onset of a symptom or inhibit onset or progression of a disease. For example, treatment may be administered to a susceptible individual prior to the onset of symptoms (e.g., in light of a history of symptoms and/or in light of genetic or other susceptibility factors). Treatment may also be continued after symptoms have resolved, for example, to prevent or delay their recurrence. In some embodiments, a treatment is administered to treat a triplet repeat disorder. In certain embodiments, a treatment is administered to treat Huntington's disease. In certain embodiments, a treatment is administered to treat Friedreich's ataxia.

### Triplet Repeat Disorder

The terms “triplet repeat disorder,” “trinucleotide repeat disorder,” “triplet repeat expansion disorder,” or “trinucleotide repeat expansion disorder refer to a number of human disease, including Huntington's Disease, Fragile X syndrome, and Friedreich's ataxia, associated with expansion of particular trinucleotide repeat sequences. The most common trinucleotide repeat contains CAG triplets, though GAA triplets (Friedreich's ataxia) and CGG triplets (Fragile X syndrome) also occur. Inheriting a predisposition to expansion, or acquiring an already expanded parental allele, increases the likelihood of acquiring the disease.

Triplet repeat disorders include, for example, those associated with the following genes and numbers of pathogenic polyglutamine trinucleotide repeats:

| | | POSITION OF |
|---|---|---|
| | | PATHOGENIC |
| TRIPLET REPEAT DISORDER | AFFECTED GENE | REPEATS |
| DENTATORUBROPALLIDOLUYSIAN | ATN1 | 49-88 |
| ATROPHY | ATROPHIN-1 | |
| HUNTINGTON'S DISEASE | HTT THE | 36-250 |
| | HUNTINGTIN | |
| | GENE | |
| SPINAL AND BULBAR MUSCULAR | AR | 38-62 |
| ATROPHY | ANDROGEN | |
| | RECEPTOR | |
| SPINOCEREBELLAR ATAXIA TYPE 1 | ATXN1 | 49-88 |
| | ATAXIN 1 | |
| SPINOCEREBELLAR ATAXIA TYPE 2 | ATXN2 | 33-77 |
| | ATAXIN 2 | |
| SPINOCEREBELLAR ATAXIA TYPE 3 | ATXN3 | 55-86 |
| | ATAXIN 3 | |
| SPINOCEREBELLAR ATAXIA TYPE 6 | CACNA1A | 21-30 |
| SPINOCEREBELLAR ATAXIA TYPE 7 | ATXN7 | 38-120 |
| | ATAXIN 7 | |
| SPINOCEREBELLAR ATAXIA TYPE 17 | TBP TATA- | 47-63 |
| | BINDING | |
| | PROTEIN | |

Triplet repeat disorders include, for example, those associated with the following genes and numbers of pathogenic non-polyglutamine trinucleotide repeats:

| | | POSITION OF |
|---|---|---|
| | | PATHOGENIC |
| TRIPLET REPEAT DISORDER | AFFECTED GENE | REPEATS |
| FRDA (FRIEDREICH'S ATAXIA) | FXN | 100+ |
| | FRATAXIN | |
| FRAXA (FRAGILE X | FMR1 | 230+ |
| SYNDROME) | FRAGILE X MENTAL | |
| | RETARDATION PROTEIN | |
| FXTAS (FRAGILE X- | FMR1 | 55-200 |
| ASSOCIATED | | |
| TREMOR/ATAXIA | | |
| SYNDROME) | | |
| FRAXE (FRAGILE XE MENTAL | AFF2 | 200+ |
| RETARDATION) | | |
| DM1 (MYOTONIC | DMPK | 50+ |
| DYSTROPHY TYPE 1) | MYOTONIN-PROTEIN | |
| | KINASE | |
| SCA8 (SPINOCEREBELLAR | SCA8 | 110-250 |
| ATAXIA TYPE 8) | ATAXIN 8 | |
| SCA12 (SPINOCEREBELLAR | PPP2R2B | 66-78 |
| ATAXIA TYPE 12) | SERINE/THREONIN | |
| | PROTEIN | |
| | PHOSPHATASE 2A | |

Trinucleotide repeat expansion disorders are complex, progressive disorders that involve developmental neurobiology and often affect cognition as well as sensory-motor functions. The disorders show genetic anticipation (i.e., increased severity with each generation). The DNA expansions or contractions usually happen meiotically (i.e., during the time of gametogenesis, or early in embryonic development), and often have sex-bias meaning that some genes expand only when inherited through the female, and others only through the male. In humans, trinucleotide repeat expansion disorders can cause gene silencing at either the transcriptional or translational level, which essentially knocks out gene function. Alternatively, trinucleotide repeat expansion disorders can cause altered proteins generated with large repetitive amino acid sequences that either abrogate or change protein function, often in a dominant-negative manner (e.g., poly-glutamine diseases).

Without wishing to be bound by theory, triplet expansion is caused by slippage during DNA replication or during DNA repair synthesis. Because the tandem repeats have identical sequence to one another, base pairing between two DNA strands can take place at multiple points along the sequence. This may lead to the formation of “loop out” structures during DNA replication or DNA repair synthesis. This may also lead to repeated copying of the repeated sequence, expanding the number of repeats. Additional mechanisms involving hybrid RNA: DNA intermediates have been proposed.

Depending on the particular trinucleotide expansion disorder, the defect-inducing triplet expansions may occur in “trinucleotide repeat expansion proteins.” Trinucleotide repeat expansion proteins are a diverse set of proteins associated with susceptibility for developing a trinucleotide repeat expansion disorder, the presence of a trinucleotide repeat expansion disorder, the severity of a trinucleotide repeat expansion disorder, or any combination thereof. Trinucleotide repeat expansion disorders are divided into two categories determined by the type of repeat. The most common repeat is the triplet CAG, which, when present in the coding region of a gene, codes for the amino acid glutamine (Q). Therefore, these disorders are referred to as the polyglutamine (polyQ) disorders and comprise the following diseases: Huntington's Disease (HD); Spinobulbar Muscular Atrophy (SBMA); Spinocerebellar Ataxias (SCA types 1, 2, 3, 6, 7, and 17); and Dentatorubro-Pallidoluysian Atrophy (DRPLA). The remaining trinucleotide repeat expansion disorders either do not involve the CAG triplet or the CAG triplet is not in the coding region of the gene. These disorders, therefore, are referred to as the non-polyglutamine disorders. The non-polyglutamine disorders comprise Fragile X Syndrome (FRAXA); Fragile XE Mental Retardation (FRAXE); Friedreich's Ataxia (FRDA); Myotonic Dystrophy (DM); and Spinocerebellar Ataxias (SCA types 8 and 12).

The production rate or circulating concentration of a protein associated with a trinucleotide repeat expansion disorder may be elevated or depressed in a population having a trinucleotide repeat expansion disorder relative to a population lacking the trinucleotide repeat expansion disorder. Non-limiting examples of proteins associated with trinucleotide repeat expansion disorders include AR (androgen receptor), FMR1 (fragile X mental retardation 1), HTT (huntingtin), DMPK (dystrophia myotonica-protein kinase), FXN (frataxin), ATXN2 (ataxin 2), ATN1 (atrophin 1), FEN1 (flap structure-specific endonuclease 1), TNRC6A (trinucleotide repeat containing 6A), PABPN1 (poly(A) binding protein, nuclear 1), JPH3 (junctophilin 3), MED15 (mediator complex subunit 15), ATXN1 (ataxin 1), ATXN3 (ataxin 3), TBP (TATA box binding protein), CACNA1A (calcium channel, voltage-dependent, P/Q type, alpha 1A subunit), ATXN80S (ATXN8 opposite strand (non-protein coding)), PPP2R2B (protein phosphatase 2, regulatory subunit B, beta), ATXN7 (ataxin 7), TNRC6B (trinucleotide repeat containing 6B), TNRC6C (trinucleotide repeat containing 6C), CELF3 (CUGBP, Elav-like family member 3), MAB21L1 (mab-21-like 1 (C. elegans)), MSH2 (mutS homolog 2, colon cancer, nonpolyposis type 1 (E. coli)), TMEM185A (transmembrane protein 185A), SIX5 (SIX homeobox 5), CNPY3 (canopy 3 homolog (zebrafish)), FRAXE (fragile site, folic acid type, rare, fra(X)(q28) E), GNB2 (guanine nucleotide binding protein (G protein), beta polypeptide 2), RPL14 (ribosomal protein L14), ATXN8 (ataxin 8), INSR (insulin receptor), TTR (transthyretin), EP400 (E1A binding protein p400), GIGYF2 (GRB10 interacting GYF protein 2), OGG1 (8-oxoguanine DNA glycosylase), STC1 (stanniocalcin 1), CNDP1 (carnosine dipeptidase 1 (metallopeptidase M20 family)), C10orf2 (chromosome 10 open reading frame 2), MAML3 mastermind-like 3 (Drosophila), DKC1 (dyskeratosis congenita 1, dyskerin), PAXIP1 (PAX interacting (with transcription-activation domain) protein 1), CASK (calcium/calmodulin-dependent serine protein kinase (MAGUK family)), MAPT (microtubule-associated protein tau), SP1 (Sp1 transcription factor), POLG (polymerase (DNA directed), gamma), AFF2 (AF4/FMR2 family, member 2), THBS1 (thrombospondin 1), TP53 (tumor protein p53), ESR1 (estrogen receptor 1), CGGBP1 (CGG triplet repeat binding protein 1), ABT1 (activator of basal transcription 1), KLK3 (kallikrein-related peptidase 3), PRNP (prion protein), JUN (jun oncogene), KCNN3 (potassium intermediate/small conductance calcium-activated channel, subfamily N, member 3), BAX (BCL2-associated X protein), FRAXA (fragile site, folic acid type, rare, fra(X)(q27.3) A (macroorchidism, mental retardation)), KBTBD10 (kelch repeat and BTB (POZ) domain containing 10), MBNL1 (muscleblind-like (Drosophila)), RAD51 (RAD51 homolog (RecA homolog, E. coli) (S. cerevisiae)), NCOA3 (nuclear receptor coactivator 3), ERDA1 (expanded repeat domain, CAG/CTG 1), TSC1 (tuberous sclerosis 1), COMP (cartilage oligomeric matrix protein), GCLC (glutamate-cysteine ligase, catalytic subunit), RRAD (Ras-related associated with diabetes), MSH3 (mutS homolog 3 (E. coli)), DRD2 (dopamine receptor D2), CD44 (CD44 molecule (Indian blood group)), CTCF (CCCTC-binding factor (zinc finger protein)), CCND1 (cyclin D1), CLSPN (claspin homolog (Xenopus laevis)), MEF2A (myocyte enhancer factor 2A), PTPRU (protein tyrosine phosphatase, receptor type, U), GAPDH (glyceraldehyde-3-phosphate dehydrogenase), TRIM22 (tripartite motif-containing 22), WT1 (Wilms tumor 1), AHR (aryl hydrocarbon receptor), GPX1 (glutathione peroxidase 1), TPMT (thiopurine S-methyltransferase), NDP (Norrie disease (pseudoglioma)), ARX (aristaless related homeobox), MUS81 (MUS81 endonuclease homolog (S. cerevisiae)), TYR (tyrosinase (oculocutaneous albinism IA)), EGR1 (early growth response 1), UNG (uracil-DNA glycosylase), NUMBL (numb homolog (Drosophila)-like), FABP2 (fatty acid binding protein 2, intestinal), EN2 (engrailed homeobox 2), CRYGC (crystallin, gamma C), SRP14 (signal recognition particle 14 kDa (homologous Alu RNA binding protein)), CRYGB (crystallin, gamma B), PDCD1 (programmed cell death 1), HOXA1 (homeobox A1), ATXN2L (ataxin 2-like), PMS2 (PMS2 postmeiotic segregation increased 2 (S. cerevisiae)), GLA (galactosidase, alpha), CBL (Cas-Br-M (murine) ecotropic retroviral transforming sequence), FTH1 (ferritin, heavy polypeptide 1), IL12RB2 (interleukin 12 receptor, beta 2), OTX2 (orthodenticle homeobox 2), HOXA5 (homeobox A5), POLG2 (polymerase (DNA directed), gamma 2, accessory subunit), DLX2 (distal-less homeobox 2), SIRPA (signal-regulatory protein alpha), OTX1 (orthodenticle homeobox 1), AHRR (aryl-hydrocarbon receptor repressor), MANF (mesencephalic astrocyte-derived neurotrophic factor), TMEM158 (transmembrane protein 158 (gene/pseudogene)), and ENSG00000078687. In certain embodiments, the protein associated with a trinucleotide repeat disorder is the HTT protein. In certain embodiments, the protein associated with a trinucleotide repeat disorder is the FXN protein.

### Variant

As used herein, the term “variant” should be taken to mean the exhibition of qualities that have a pattern that deviates from what occurs in nature, e.g., a variant Cas9 is a Cas9 comprising one or more changes in amino acid residues as compared to a wild type Cas9 amino acid sequence. The term “variant” encompasses homologous proteins having at least 75%, at least 80%, at least 85%, at least 90%, at least 95%, or at least 99% identity with a reference sequence and having the same or substantially the same functional activity or activities as the reference sequence. The term also encompasses mutants, truncations, or domains of a reference sequence that display the same or substantially the same functional activity or activities as the reference sequence.

### Vector

The term “vector,” as used herein, refers to a nucleic acid that can be modified to encode a gene of interest and that is able to enter a host cell, mutate, and replicate within the host cell, and then transfer a replicated form of the vector into another host cell. Exemplary suitable vectors include viral vectors, such as retroviral vectors or bacteriophages and filamentous phage, and conjugative plasmids. Additional suitable vectors will be apparent to those of skill in the art based on the instant disclosure.

### Wild Type

As used herein the term “wild type” is a term of the art understood by skilled persons and means the typical form of an organism, strain, gene, or characteristic as it occurs in nature as distinguished from mutant or variant forms.

### DETAILED DESCRIPTION

The present disclosure provides compositions, systems, and methods useful in the treatment of trinucleotide repeat disorders, including Huntington's disease and Friedreich's ataxia. The present disclosure also provides pegRNAs designed to target the HTT or FXN genes. Complexes comprising a prime editor and any of the pegRNAs disclosed herein are also provided by the present disclosure. The present disclosure further provides polynucleotides, vectors, AAVs, cells, compositions, and kits. Methods of treating Huntington's disease and Friedreich's ataxia, as well as uses of the compositions, pegRNAs, and systems described herein, are also provided herein.

### PEgRNAs

The prime editing complexes and methods described herein contemplate the use of any suitable PEgRNAs. In various aspects, the present disclosure provides pegRNAs targeting the HTT and FXN genes as described herein. Such pegRNAs may be useful, for example, in prime editing methods targeting pathogenic trinucleotide repeats in HTT and FXN for the treatment of Huntington's disease and Friedreich's ataxia. In some embodiments, a trinucleotide repeat is replaced with a trinucleotide repeat of a different length using the pegRNAs provided herein. In some embodiments, a trinucleotide repeat is contracted using the pegRNAs provided herein. In some embodiments, a trinucleotide repeat is deleted using the pegRNAs provided herein. In certain embodiments, CAG repeats in the HTT gene (e.g., at the 5′-end of the HTT gene) are replaced, contracted, or deleted using the pegRNAs provided herein. In certain embodiments, GAA repeats in the FXN gene (e.g., in intron 1 of the FXN gene) are replaced, contracted, or deleted using the pegRNAs provided herein.

In one aspect, the present disclosure provides pegRNAs targeting the HTT gene comprising a spacer sequence comprising the nucleic acid sequence: GACCCTGGAAAAGCTGATGA (SEQ ID NO: 381); GCTGCTGCTGGAAGGACTTG (SEQ ID NO: 382); GCTGCTGCTGCTGCTGCTGGA (SEQ ID NO: 383); GCTGCTGCTGCTGCTGCTGC (SEQ ID NO: 384); GGCGGCGGCGGCGGCGGTGG (SEQ ID NO: 385); TGAGGAAGCTGAGGAGGCGG (SEQ ID NO: 386); or GGCGGCTGAGGAAGCTGAGG (SEQ ID NO: 387), or a nucleic acid sequence at least 80%, at least 85%, at least 90%, at least 95%, at least 96%, at least 97%, at least 98%, or at least 99% identical to any one of SEQ ID NOs: 381-387. In certain embodiments, the pegRNA comprises the spacer sequence GACCCTGGAAAAGCTGATGA (SEQ ID NO: 381), or a spacer sequence at least 80%, at least 85%, at least 90%, at least 95%, at least 96%, at least 97%, at least 98%, or at least 99% identical to SEQ ID NO: 381.

In some embodiments, the pegRNA comprises a primer binding site (PBS) of about 8 to about 16 nucleotides in length. In some embodiments, the pegRNA comprises a PBS of about 8, about 9, about 10, about 11, about 12, about 13, about 14, about 15, or about 16 nucleotides in length. In certain embodiments, the pegRNA comprises a PBS of about 10 nucleotides in length.

In some embodiments, the pegRNA comprises a reverse transcription template comprising a sequence of about 15 to about 35 nucleotides, wherein the sequence encodes a sequence that comprises one or more nucleotide edits compared to a sequence directly upstream of a CAG repeat in exon 1 of the HTT gene. In some embodiments, the one or more nucleotide edits comprises a PAM mutation that alters a PAM sequence. In certain embodiments, the PAM sequence is NGG, wherein N is any one of nucleotides A, G, C, or T. In certain embodiments, the pegRNA comprises a reverse transcription template comprising a sequence of about 26 nucleotides, wherein the sequence encodes a sequence that comprises one or more nucleotide edits compared to a sequence directly upstream of a CAG repeat in exon 1 of the HTT gene. In some embodiments, the pegRNA comprises a reverse transcription template comprising an edit template encoding or comprising one or repeats of the trinucleotide sequence CAG (for example, 4-35 or 4-10 repeats). In certain embodiments, the pegRNA comprises a reverse transcription template comprising an edit template encoding or comprising the nucleotide sequence CAGCAGCAGCAG (SEQ ID NO: 874), or a nucleotide sequence at least 80%, at least 85%, at least 90%, at least 95%, at least 96%, at least 97%, at least 98%, or at least 99% identical to SEQ ID NO: 874. In certain embodiments, the pegRNA comprises a reverse transcription template comprising an edit template comprising the nucleotide sequence CAGCAGCAGCAGCAGCAGCAGCAGCAG (SEQ ID NO: 875), or a nucleotide sequence at least 80%, at least 85%, at least 90%, at least 95%, at least 96%, at least 97%, at least 98%, or at least 99% identical to SEQ ID NO: 875. In some embodiments, the edit template further comprises one or more nucleotides of the trinucleotide sequence CAA. In certain embodiments, the pegRNA comprises a reverse transcription template comprising an edit template comprising the nucleotide sequence CAGCAGCAGCAGCAACAACAA (SEQ ID NO: 876), or a nucleotide sequence at least 80%, at least 85%, at least 90%, at least 95%, at least 96%, at least 97%, at least 98%, or at least 99% identical to SEQ ID NO: 876. In some embodiments, the pegRNA comprises a reverse transcription template comprising a homology arm of about 16 to about 50 nucleotides in length, wherein the homology arm is complementary to a sequence directly downstream of a CAG repeat in exon 1 of a wildtype HTT gene in the coding strand. In certain embodiments, the pegRNA comprises a reverse transcription template comprising a homology arm of about 31 nucleotides in length. In certain embodiments, the pegRNA comprises a reverse transcription template comprising a homology arm of about 40 nucleotides in length.

In some embodiments, the pegRNA for editing HTT is an engineered pegRNA (epegRNA). In some embodiments, the pegRNA comprises at its 3′ end a structural motif that improves stability of the pegRNA. In some embodiments, the epegRNA comprises an evopreq1 motif. In certain embodiments, the evopreq1 motif comprises the sequence of SEQ ID NO: 442, or a sequence at least 80%, at least 85%, at least 90%, at least 95%, at least 96%, at least 97%, at least 98%, or at least 99% identical to SEQ ID NO: 442, a sequence at least 80%, at least 85%, at least 90%, at least 95%, at least 96%, at least 97%, at least 98%, or at least 99% identical to the sequence of SEQ ID NO: 442, or a sequence having 1, 2, 3, 4, or 5 mutations relative to SEQ ID NO: 442. In some embodiments, the pegRNA comprises a UA flip in the pegRNA scaffold sequence. In certain embodiments, the pegRNA comprises the sequence of any one of SEQ ID NOs: 454-815, or a sequence at least 80%, at least 85%, at least 90%, at least 95%, at least 96%, at least 97%, at least 98%, or at least 99% identical to the sequence of any one of SEQ ID NOs: 454-815.

In another aspect, the present disclosure provides pegRNAs targeting the FXN gene comprising a spacer sequence comprising the nucleic acid sequence: GCAAGACTAACCTGGCCAACA (SEQ ID NO: 388); GTCCGGAGTTCAAGACTAACC (SEQ ID NO: 389); GAAGGTGGATCACCTGAGGTC (SEQ ID NO: 390); GTCTGGAGTAGCTGGGATTAC (SEQ ID NO: 391); or GCAGGCGCGCGACACCACGCC (SEQ ID NO: 392), or a nucleic acid sequence at least 80%, at least 85%, at least 90%, at least 95%, at least 96%, at least 97%, at least 98%, or at least 99% identical to any one of SEQ ID NOs: 388-392. In certain embodiments, the pegRNA comprises the spacer sequence GCAAGACTAACCTGGCCAACA (SEQ ID NO: 388), or a nucleic acid sequence at least 80%, at least 85%, at least 90%, at least 95%, at least 96%, at least 97%, at least 98%, or at least 99% identical to SEQ ID NO: 388.

In some embodiments, the pegRNA comprises a primer binding site (PBS) of about 8 to about 14 nucleotides in length. In some embodiments, the pegRNA comprises a PBS of about 8, about 9, about 10, about 11, about 12, about 13, or about 14 nucleotides in length. In certain embodiments, the pegRNA comprises a PBS of about 10 nucleotides in length.

In some embodiments, the pegRNA comprises a reverse transcription template comprising a homology arm of about 8 to about 50 nucleotides in length, wherein the homology arm is complementary to a sequence directly downstream of a GAA repeat in a wildtype FXN gene. In certain embodiments, the pegRNA comprises a reverse transcription template comprising a homology arm of about 40 nucleotides in length.

In some embodiments, the pegRNA for editing FXN is an engineered pegRNA (epegRNA). In some embodiments, the pegRNA comprises at its 3′ end a structural motif that improves stability of the pegRNA. In some embodiments, the epegRNA comprises an evopreq1 motif. In certain embodiments, the evopreq1 motif comprises the sequence of SEQ ID NO: 442, a sequence at least 80%, at least 85%, at least 90%, at least 95%, at least 96%, at least 97%, at least 98%, or at least 99% identical to the sequence of SEQ ID NO: 442, or a sequence having 1, 2, 3, 4, or 5 mutations relative to SEQ ID NO: 442. In some embodiments, the pegRNA comprises a UA flip in the pegRNA scaffold sequence.

In certain embodiments, the pegRNA for editing FXN comprises the sequence of any one of SEQ ID NOs: 816-867, or a sequence at least 80%, at least 85%, at least 90%, at least 95%, at least 96%, at least 97%, at least 98%, or at least 99% identical to the sequence of any one of SEQ ID NOs: 816-867.

In some aspects, the present disclosure also provides gRNAs for nicking the non-PAM-containing strand of the target nucleotide sequence. In some embodiments, the spacer of the nicking gRNA targets the HTT gene and comprises the nucleotide sequence: GGCGGCTGAGGAAGCTGAGG (SEQ ID NO: 387); GGCGGCGGCGGCGGCGGTGG (SEQ ID NO: 385); GCTGCTGCTGCTGCTGCTGC (SEQ ID NO: 384); GAAGGACTTGAGGGACTCGA (SEQ ID NO: 393); GTGAGGAAGCTGAGGAGGCGG (SEQ ID NO: 394); GCTGTTGCTGCTGCTGCTGC (SEQ ID NO: 395); GCTGCTGCTGCTGCTGCTGGA (SEQ ID NO: 383); GCTGCTGCTGGAAGGACTTG (SEQ ID NO: 382); GGCCTTCATCAGCTTTTCC (SEQ ID NO: 396); GGCTTTCATCAGCTTTTCC (SEQ ID NO: 397); GGGACTCGAAGGCCTTCAT (SEQ ID NO: 398); GGAAGGACTTGAGGGACTCG (SEQ ID NO: 399); GCTGCTGCTGGAAGGACTT (SEQ ID NO: 400); GCTGCTGCTGCTGCTGCTGG (SEQ ID NO: 401); GCTGGAAGGACTTGAGGGACT (SEQ ID NO: 402); GCTGCTGCTGCTGGAAGGACT (SEQ ID NO: 403); CTGCTGCTGCTGCTGCTGGAA (SEQ ID NO: 404); GCTGCTGCTGCTGCTGCTGCT (SEQ ID NO: 405); GCTGCTGGAAGGACTTGAG (SEQ ID NO: 406); GCTGCTGGAAGGACTTCAG (SEQ ID NO: 407); GCTGCTGAAAGGACTTCAG (SEQ ID NO: 408); or GCTGTTGGAAGGACTTCAG (SEQ ID NO: 409), or a nucleotide sequence at least 80%, at least 85%, at least 90%, at least 95%, at least 96%, at least 97%, at least 98%, or at least 99% identical to any of these. In certain embodiments, the spacer of the nicking gRNA comprises the nucleotide sequence GCTGCTGCTGCTGCTGCTGC (SEQ ID NO: 384), or a nucleotide sequence at least 80%, at least 85%, at least 90%, at least 95%, at least 96%, at least 97%, at least 98%, or at least 99% identical to SEQ ID NO: 384. In certain embodiments, the spacer of the nicking gRNA comprises the nucleotide sequence GCTGCTGGAAGGACTTGAG (SEQ ID NO: 406), or a nucleotide sequence at least 80%, at least 85%, at least 90%, at least 95%, at least 96%, at least 97%, at least 98%, or at least 99% identical to SEQ ID NO: 406.

In some embodiments, the spacer of the nicking gRNA targets the FXN gene and comprises the nucleotide sequence: GTCCCAAAGTGCTGAGATTAT (SEQ ID NO: 410); GTGTATTTTTTAGTAGATACT (SEQ ID NO: 411); GATTCTCCTGCCGCAGCCTC (SEQ ID NO: 412); or GCGACACCACGCCCGGCTAAC (SEQ ID NO: 413), or a nucleotide sequence at least 80%, at least 85%, at least 90%, at least 95%, at least 96%, at least 97%, at least 98%, or at least 99% identical to any of these. In certain embodiments, the spacer of the nicking gRNA comprises the nucleotide sequence GTGTATTTTTTAGTAGATACT (SEQ ID NO: 411), or a nucleotide sequence at least 80%, at least 85%, at least 90%, at least 95%, at least 96%, at least 97%, at least 98%, or at least 99% identical to SEQ ID NO: 411. In certain embodiments, the spacer of the nicking gRNA comprises the nucleotide sequence GCGACACCACGCCCGGCTAAC (SEQ ID NO: 413), or a nucleotide sequence at least 80%, at least 85%, at least 90%, at least 95%, at least 96%, at least 97%, at least 98%, or at least 99% identical to SEQ ID NO: 413.

As described further below, various gRNA core sequences may be utilized in the pegRNAs provided herein. In some embodiments, a gRNA core that binds to SpCas9 is used in the pegRNAs provided herein. In some embodiments, a gRNA core that binds to SaCas9 is used in the pegRNAs provided herein. In some embodiments, a gRNA core that binds to any Cas9 protein provided herein, or any Cas9 protein known in the art, is used in the pegRNAs provided herein.

### PEgRNA Architecture

In some embodiments, an extended guide RNA, or pegRNA, used in the prime editing complexes and methods disclosed herein includes a spacer sequence (e.g., a ~20 nt spacer sequence) and a gRNA core region, which binds with the napDNAbp. In some embodiments, the pegRNA includes an extended RNA segment, i.e., an extension arm, at the 5′ end, i.e., a 5′ extension. In some embodiments, the 5′ extension includes a reverse transcription template sequence, a primer binding site, and an optional 5-20 nucleotide linker sequence. The RT primer binding site hybridizes to the free 3′ end that is formed after a nick is formed in the non-target strand of the R-loop, thereby priming reverse transcriptase for DNA polymerization in the 5′-3′ direction.

In another embodiment, an extended guide RNA (i.e., a pegRNA) used in the prime editing complexes and methods provided herein includes a spacer sequence (e.g., a ~20 nt spacer sequence) and a gRNA core, which binds with the napDNAbp. In some embodiments, the pegRNA includes an extended RNA segment, i.e., an extension arm, at the 3′ end, i.e., a 3′ extension. In some embodiments, the 3′ extension includes a reverse transcription template sequence, and a reverse transcription primer binding site. The RT primer binding site hybridizes to the free 3′ end that is formed after a nick is formed in the non-target strand of the R-loop, thereby priming reverse transcriptase for DNA polymerization in the 5′-3′ direction.

In another embodiment, an extended guide RNA (i.e., a pegRNA) used in the prime editing complexes and methods provided herein includes a spacer sequence (e.g., a ~20 nt spacer sequence) and a gRNA core, which binds with the napDNAbp. In some embodiments, the pegRNA includes an extended RNA segment, i.e., an extension arm, at an intermolecular position within the gRNA core, i.e., an intramolecular extension. In some embodiments, the intramolecular extension includes a reverse transcription template sequence, and a reverse transcription primer binding site. The RT primer binding site hybridizes to the free 3′ end that is formed after a nick is formed in the non-target strand of the R-loop, thereby priming reverse transcriptase for DNA polymerization in the 5′-3′ direction.

In one embodiment, the position of the intermolecular RNA extension is not in the spacer sequence of the guide RNA. In another embodiment, the position of the intermolecular RNA extension is in the gRNA core. In still another embodiment, the position of the intermolecular RNA extension is anywhere within the guide RNA molecule except within the spacer sequence, or at a position which disrupts the spacer sequence. In one embodiment, the intermolecular RNA extension is inserted downstream from the 3′ end of the spacer sequence. In another embodiment, the intermolecular RNA extension is inserted at least 1 nucleotide, at least 2 nucleotides, at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 21 nucleotides, at least 22 nucleotides, at least 23 nucleotides, at least 24 nucleotides, or at least 25 nucleotides downstream of the 3′ end of the spacer sequence.

In other embodiments, the intermolecular RNA extension is inserted into the gRNA core, which refers to the portion of a traditional guide RNA corresponding or comprising the tracrRNA, which binds and/or interacts with the napDNAbp, e.g., a Cas9 protein or equivalent thereof (i.e., a different napDNAbp). Preferably the insertion of the intermolecular RNA extension does not disrupt or minimally disrupts the interaction between the tracrRNA portion and the napDNAbp.

The length of the RNA extension (which includes at least the RT template and primer binding site) can be any useful length. In various embodiments, the RNA extension is at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 21 nucleotides, at least 22 nucleotides, at least 23 nucleotides, at least 24 nucleotides, at least 25 nucleotides, at least 30 nucleotides, at least 40 nucleotides, at least 50 nucleotides, at least 60 nucleotides, at least 70 nucleotides, at least 80 nucleotides, at least 90 nucleotides, at least 100 nucleotides, at least 200 nucleotides, at least 300 nucleotides, at least 400 nucleotides, or at least 500 nucleotides in length.

The RT template sequence can also be any suitable length. For example, the RT template sequence can be at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 30 nucleotides, at least 40 nucleotides, at least 50 nucleotides, at least 60 nucleotides, at least 70 nucleotides, at least 80 nucleotides, at least 90 nucleotides, at least 100 nucleotides, at least 200 nucleotides, at least 300 nucleotides, at least 400 nucleotides, or at least 500 nucleotides in length.

In still other embodiments, the reverse transcription primer binding site sequence is at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 30 nucleotides, at least 40 nucleotides, at least 50 nucleotides, at least 60 nucleotides, at least 70 nucleotides, at least 80 nucleotides, at least 90 nucleotides, at least 100 nucleotides, at least 200 nucleotides, at least 300 nucleotides, at least 400 nucleotides, or at least 500 nucleotides in length.

In other embodiments, the optional linker or spacer sequence is at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 30 nucleotides, at least 40 nucleotides, at least 50 nucleotides, at least 60 nucleotides, at least 70 nucleotides, at least 80 nucleotides, at least 90 nucleotides, at least 100 nucleotides, at least 200 nucleotides, at least 300 nucleotides, at least 400 nucleotides, or at least 500 nucleotides in length.

The RT template sequence, in certain embodiments, encodes a single-stranded DNA molecule which is homologous to the non-target strand (and thus, complementary to the corresponding site of the target strand) but includes one or more nucleotide changes. The one or more nucleotide changes may include one or more single-base nucleotide changes, one or more deletions, and/or one or more insertions.

The synthesized single-stranded DNA product of the RT template sequence is homologous to the non-target strand except that it contains one or more nucleotide changes. The single-stranded DNA product of the RT template sequence hybridizes in equilibrium with the complementary target strand sequence, thereby displacing the homologous endogenous target strand sequence. The displaced endogenous strand may be referred to in some embodiments as a 5′ endogenous DNA flap species. This 5′ endogenous DNA flap species can be removed by a 5′ flap endonuclease (e.g., FEN1) and the single-stranded DNA product, now hybridized to the endogenous target strand, may be ligated, thereby creating a mismatch between the endogenous sequence and the newly synthesized strand. The mismatch may be resolved by the cell's innate DNA repair and/or replication processes.

In various embodiments, the nucleotide sequence of the RT template sequence corresponds to the nucleotide sequence of the non-target strand that becomes displaced as the 5′ flap species and that overlaps with the site to be edited.

In various embodiments of the extended guide RNAs, the reverse transcription template sequence may encode a single-strand DNA flap that is complementary to an endogenous DNA sequence adjacent to a nick site, wherein the single-strand DNA flap comprises a desired nucleotide change. The single-stranded DNA flap may displace an endogenous single-strand DNA at the nick site. The displaced endogenous single-strand DNA at the nick site can have a 5′ end and form an endogenous flap, which can be excised by the cell. In various embodiments, excision of the 5′ end endogenous flap can help drive product formation since removing the 5′ end endogenous flap encourages hybridization of the single-strand 3′ DNA flap to the corresponding complementary DNA strand, and the incorporation or assimilation of the desired nucleotide change carried by the single-strand 3′ DNA flap into the target DNA.

The terms “cleavage site,” “nick site,” and “cut site,” as used interchangeably herein in the context of prime editing, refer to a specific position in between two nucleotides or two base pairs in the double-stranded target DNA sequence. In some embodiments, the position of a nick site is determined relative to the position of a specific PAM sequence. In some embodiments, the nick site is the particular position where a nick will occur when the double stranded target DNA is contacted with a napDNAbp, e.g., a nickase such as a Cas nickase, that recognizes a specific PAM sequence. For each PEgRNA described herein, a nick site (e.g., the “first nick site” when referred to in the context of PE3, PE5 and similar approaches), is characteristic of the particular napDNAbp to which the gRNA core of the PEgRNA associates with, and is characteristic of the particular PAM required for recognition and function of the napDNAbp. For example, for a PEgRNA that comprises a gRNA core that associates with a SpCas9, the nick site in the phosphodiester bond between bases three (“−3” position relative to the position 1 of the PAM sequence) and four (“−4” position relative to position 1 of the PAM sequence).

In some embodiments, a nick site is in a target strand of the double-stranded target DNA sequence. In some embodiments, a nick site is in a non-target strand of the double-stranded target DNA sequence. In some embodiments, the nick site is in a protospacer sequence. In some embodiments, the nick site is adjacent to a protospacer sequence. In some embodiments, a nick site is downstream of a region, e.g., on a non-target strand, that is complementary to a primer binding site of a PEgRNA. In some embodiments, a nick site is downstream of a region, e.g., on a non-target strand, that binds to a primer binding site of a PEgRNA. In some embodiments, a nick site is immediately downstream of a region, e.g., on a non-target strand, that is complementary to a primer binding site of a PERNA. In some embodiments, the nick site is upstream of a specific PAM sequence on the non-target strand of the double stranded target DNA, wherein the PAM sequence is specific for recognition by a napDNAbp that associates with the gRNA core of a PEgRNA. In some embodiments, the nick site is downstream of a specific PAM sequence on the non-target strand of the double stranded target DNA, wherein the PAM sequence is specific for recognition by a napDNAbp that associates with the gRNA core of a PEgRNA. In some embodiments, the nick site is 3 nucleotides upstream of the PAM sequence, and the PAM sequence is recognized by a Streptococcus pyogenes Cas9 nickase, a P. lavamentivorans Cas9 nickase, a C. diphtheriae Cas9 nickase, a N. cinerea Cas9, a S. aureus Cas9, or a N. lari Cas9 nickase. In some embodiments, the nick site is 3 nucleotides upstream of the PAM sequence, and the PAM sequence is recognized by a Cas9 nickase, wherein the Cas9 nickase comprises a nuclease active HNH domain and a nuclease inactive RuvC domain. In some embodiments, the nick site is 2 base pairs upstream of the PAM sequence, and the PAM sequence is recognized by a S. thermophilus Cas9 nickase.

In various embodiments of the extended guide RNAs, the cellular repair of the single-strand DNA flap results in installation of the desired nucleotide change, thereby forming a desired product.

In still other embodiments, the desired nucleotide change is installed in an editing window that is between about −5 to +5 of the nick site, or between about −10 to +10 of the nick site, or between about −20 to +20 of the nick site, or between about −30 to +30 of the nick site, or between about −40 to +40 of the nick site, or between about −50 to +50 of the nick site, or between about −60 to +60 of the nick site, or between about −70 to +70 of the nick site, or between about −80 to +80 of the nick site, or between about −90 to +90 of the nick site, or between about −100 to +100 of the nick site, or between about −200 to +200 of the nick site.

In other embodiments, the desired nucleotide change is installed in an editing window that is between about +1 to +2 from the nick site, or about +1 to +3, +1 to +4, +1 to +5, +1 to +6, +1 to +7, +1 to +8, +1 to +9, +1 to +10, +1 to +11, +1 to +12, +1 to +13, +1 to +14, +1 to +15, +1 to +16, +1 to +17, +1 to +18, +1 to +19, +1 to +20, +1 to +21, +1 to +22, +1 to +23, +1 to +24, +1 to +25, +1 to +26, +1 to +27, +1 to +28, +1 to +29, +1 to +30, +1 to +31, +1 to +32, +1 to +33, +1 to +34, +1 to +35, +1 to +36, +1 to +37, +1 to +38, +1 to +39, +1 to +40, +1 to +41, +1 to +42, +1 to +43, +1 to +44, +1 to +45, +1 to +46, +1 to +47, +1 to +48, +1 to +49, +1 to +50, +1 to +51, +1 to +52, +1 to +53, +1 to +54, +1 to +55, +1 to +56, +1 to +57, +1 to +58, +1 to +59, +1 to +60, +1 to +61, +1 to +62, +1 to +63, +1 to +64, +1 to +65, +1 to +66, +1 to +67, +1 to +68, +1 to +69, +1 to +70, +1 to +71, +1 to +72, +1 to +73, +1 to +74, +1 to +75, +1 to +76, +1 to +77, +1 to +78, +1 to +79, +1 to +80, +1 to +81, +1 to +82, +1 to +83, +1 to +84, +1 to +85, +1 to +86, +1 to +87, +1 to +88, +1 to +89, +1 to +90, +1 to +90, +1 to +91, +1 to +92, +1 to +93, +1 to +94, +1 to +95, +1 to +96, +1 to +97, +1 to +98, +1 to +99, +1 to +100, +1 to +101, +1 to +102, +1 to +103, +1 to +104, +1 to +105, +1 to +106, +1 to +107, +1 to +108, +1 to +109, +1 to +110, +1 to +111, +1 to +112, +1 to +113, +1 to +114, +1 to +115, +1 to +116, +1 to +117, +1 to +118, +1 to +119, +1 to +120, +1 to +121, +1 to +122, +1 to +123, +1 to +124, or +1 to +125 from the nick site.

In still other embodiments, the desired nucleotide change is installed in an editing window that is between about +1 to +2 from the nick site, or about +1 to +5, +1 to +10, +1 to +15, +1 to +20, +1 to +25, +1 to +30, +1 to +35, +1 to +40, +1 to +45, +1 to +50, +1 to +55, +1 to +100, +1 to +105, +1 to +110, +1 to +115, +1 to +120, +1 to +125, +1 to +130, +1 to +135, +1 to +140, +1 to +145, +1 to +150, +1 to +155, +1 to +160, +1 to +165, +1 to +170, +1 to +175, +1 to +180, +1 to +185, +1 to +190, +1 to +195, or +1 to +200, from the nick site.

In various aspects, the extended guide RNAs are modified versions of an extended guide RNA. pegRNAs (i.e., extended guide RNAs) and ngRNAs may be expressed from an encoding nucleic acid, or synthesized chemically. Methods are well known in the art for obtaining or otherwise synthesizing guide RNAs, and for determining the appropriate sequence of the pegRNA, including the protospacer sequence, which interacts and hybridizes with the target strand of a genomic target site of interest.

In various embodiments, the particular design aspects of a pegRNA sequence and ngRNA sequence will depend upon the nucleotide sequence of a genomic target site of interest (i.e., the desired site to be edited) and the type of napDNAbp (e.g., Cas9 protein) present in the prime editing systems utilized in the methods and compositions described herein, among other factors, such as PAM sequence locations, percent G/C content in the target sequence, the degree of microhomology regions, secondary structures, etc.

In general, a spacer sequence (i.e., a guide sequence) of a pegRNA or ngRNA can be any polynucleotide sequence having sufficient complementarity with a target polynucleotide sequence to hybridize with the target sequence and direct sequence-specific binding of a napDNAbp (e.g., a Cas9, Cas9 homolog, or Cas9 variant) to the target sequence. In some embodiments, the degree of complementarity between a spacer and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting examples of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net). In some embodiments, a spacer is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length.

In some embodiments, a spacer is less than about 75, 50, 45, 40, 35, 30, 25, 20, 15, 12, or fewer nucleotides in length. The ability of a spacer to direct sequence-specific binding of a prime editor to a target sequence may be assessed by any suitable assay. For example, the components of a prime editor, including the spacer to be tested, may be provided to a host cell having the corresponding target sequence, such as by transfection with vectors encoding the components of a prime editor disclosed herein, followed by an assessment of preferential cleavage within the target sequence, such as by Surveyor assay as described herein. Similarly, cleavage of a target polynucleotide sequence may be evaluated in a test tube by providing the target sequence, components of a prime editor, including the spacer to be tested and a control spacer different from the test spacer, and comparing binding or rate of cleavage at the target sequence between the test and control spacer reactions. Other assays are possible, and will occur to those skilled in the art.

A spacer may be selected to target any target sequence. In some embodiments, the target sequence is a sequence within a genome of a cell. Exemplary target sequences include those that are unique in the target genome. For example, for the S. pyogenes Cas9, a unique target sequence in a genome may include a Cas9 target site of the form MMMMMMMMNNNNNNNNNNNNXGG where NNNNNNNNNNNNXGG (N is A, G, T, or C; and X can be anything). A unique target sequence in a genome may include an S. pyogenes Cas9 target site of the form MMMMMMMMMNNNNNNNNNNNXGG where NNNNNNNNNNNXGG (Nis A, G, T, or C; and X can be anything). For the S. thermophilus CRISPR1Cas9, a unique target sequence in a genome may include a Cas9 target site of the form MMMMMMMMNNNNNNNNNNNNXXAGAAW where NNNNNNNNNNNNXXAGAAW (N is A, G, T, or C; X can be anything; and W is A or T). A unique target sequence in a genome may include an S. thermophilus CRISPR 1 Cas9 target site of the form MMMMMMMMMNNNNNNNNNNNXXAGAAW where NNNNNNNNNNNXXAGAAW (N is A, G, T, or C; X can be anything; and W is A or T). For the S. pyogenes Cas9, a unique target sequence in a genome may include a Cas9 target site of the form MMMMMMMMNNNNNNNNNNNNXGGXG where NNNNNNNNNNNNXGGXG (N is A, G, T, or C; and X can be anything). A unique target sequence in a genome may include an S. pyogenes Cas9 target site of the form MMMMMMMMMNNNNNNNNNNNXGGXG where NNNNNNNNNNNXGGXG (N is A, G, T, or C; and X can be anything). In each of these sequences, “M” may be A, G, T, or C, and need not be considered in identifying a sequence as unique. In some embodiments, a target sequence is in the HTT gene. In certain embodiments, CAG repeats in the HTT gene (e.g., at the 5′-end of the HTT gene) are targeted. In some embodiments, a target sequence is in the FXN gene. In certain embodiments, GAA repeats in the FXN gene (e.g., in intron 1 of the FXN gene) are targeted.

In some embodiments, a spacer is selected to reduce the degree of secondary structure within the spacer. Secondary structure may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see, e.g., A. R. Gruber et al., 2008, Cell 106(1): 23-24; and PA Carr and GM Church, 2009, Nature Biotechnology 27(12): 1151-62). Further algorithms may be found in U.S. Application Ser. No. 61/836,080, incorporated herein by reference. In some embodiments, silent mutations are introduced in a spacer in order to alter its secondary structure and increase the efficiency of prime editing.

In some embodiments, the scaffold or gRNA core portion of a pegRNA comprises sequences corresponding to the tracr sequence and tracr mate sequence of a traditional guide RNA. In general, a tracr mate sequence includes any sequence that has sufficient complementarity with a tracr sequence to promote one or more of: (1) excision of a spacer flanked by tracr mate sequences in a cell containing the corresponding tracr sequence; and (2) formation of a complex at a target sequence, wherein the complex comprises the tracr mate sequence hybridized to the tracr sequence. In general, degree of complementarity is with reference to the optimal alignment of the tracr mate sequence and tracr sequence, along the length of the shorter of the two sequences. Optimal alignment may be determined by any suitable alignment algorithm, and may further account for secondary structures, such as self-complementarity within either the tracr sequence or tracr mate sequence. In some embodiments, the degree of complementarity between the tracr sequence and tracr mate sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and tracr mate sequence are contained within a single transcript, such that hybridization between the two produces a transcript having a secondary structure, such as a hairpin. Preferred loop forming sequences for use in hairpin structures are four nucleotides in length, and most preferably have the sequence GAAA. However, longer or shorter loop sequences may be used, as may alternative sequences. The sequences preferably include a nucleotide triplet (for example, AAA), and an additional nucleotide (for example C or G). Examples of loop forming sequences include CAAA and AAAG. In an embodiment of the invention, the transcript or transcribed polynucleotide sequence has at least two or more hairpins. In preferred embodiments, the transcript has two, three, four or five hairpins. In a further embodiment of the invention, the transcript has at most five hairpins. In some embodiments, the single transcript further includes a transcription termination sequence; preferably this is a polyT sequence, for example six T nucleotides. Further non-limiting examples of single polynucleotides comprising a spacer, a tracr mate sequence, and a tracr sequence are as follows (listed 5′ to 3′), where “N” represents a base of a spacer, the first block of lower case letters represent the tracr mate sequence, and the second block of lower case letters represent the tracr sequence, and the final poly-T sequence represents the transcription terminator:

(1)
(SEQ ID NO: 137)
NNNNNNNNGTTTTTGTACTCTCAAGATTTAGAAATAAATCTTGCAGAAGC
TACAAAGATAAGGCTTCATGCCGAAATCAACACCCTGTCATTTTATGGCA
GGGTGTTTTCGTTATTTAATTTTTT;
(2)
(SEQ ID NO: 138)
NNNNNNNNNNNNNNNNNNGTTTTTGTACTCTCAGAAATGCAGAAGCTACA
AAGATAAGGCTTCATGCCGAAATCAACACCCTGTCATTTTATGGCAGGGT
GTTTTCGTTATTTAATTTTTT;
(3)
(SEQ ID NO: 139)
NNNNNNNNNNNNNNNNNNNNGTTTTTGTACTCTCAGAAATGCAGAAGCTA
CAAAGATAAGGCTTCATGCCGAAATCAACACCCTGTCATTTTATGGCAGG
GTGTTTTTT;
(4)
(SEQ ID NO: 140)
NNNNNNNNNNNNNNNNNNNNGTTTTAGAGCTAGAAATAGCAAGTTAAAAT
AAGGCTAGTCCGTTATCAACTTGAAAAAGTGGCACCGAGTCGGTGCTTTT
TT;
(5)
(SEQ ID NO: 141)
NNNNNNNNNNNNNNNNNNNNGTTTTAGAGCTAGAAATAGCAAGTTAAAAT
AAGGCTAGTCCGTTATCAACTTGAAAAAGTGTTTTTTT;
and
(6)
(SEQ ID NO: 142)
NNNNNNNNNNNNNNNNNNNNGTTTTAGAGCTAGAAATAGCAAGTTAAAAT
AAGGCTAGTCCGTTATCATTTTTTTT

In some embodiments, sequences (1) to (3) are used in combination with Cas9 from S. thermophilus CRISPR1. In some embodiments, sequences (4) to (6) are used in combination with Cas9 from S. pyogenes. In some embodiments, the tracr sequence is a separate transcript from a transcript comprising the tracr mate sequence.

It will be apparent to those of skill in the art that in order to target any of the fusion proteins comprising a Cas9 domain and a single-stranded DNA binding protein, as disclosed herein, to a target site, e.g., a site comprising a point mutation to be edited, it is typically necessary to co-express the fusion protein together with a guide RNA, e.g., an sgRNA. As explained in more detail elsewhere herein, a guide RNA typically comprises a tracrRNA framework allowing for Cas9 binding, and a spacer, which confers sequence specificity to the Cas9: nucleic acid editing enzyme/domain fusion protein.

In some embodiments, a pegRNA comprises a structure 5′-[spacer]-GUUUUAGAGCUAGAAAUAGCAAGUUAAAAUAAAGGCUAGUCCGUUAUCAACU UGAAAAAGUGGCACCGAGUCGGUGCUUUUU (SEQ ID NO: 143)-extension arm-3′, wherein the spacer comprises a sequence that is complementary to the target sequence. The spacer, also referred to herein as the spacer sequence, is typically 20 nucleotides long. The sequences of suitable guide RNAs for targeting Cas9: nucleic acid editing enzyme/domain fusion proteins to specific genomic target sites will be apparent to those of skill in the art based on the instant disclosure. Such suitable guide RNA sequences typically comprise spacers that are complementary to a nucleic acid sequence within 50 nucleotides upstream or downstream of the target nucleotide to be edited. Some exemplary guide RNA sequences suitable for targeting any of the provided fusion proteins to specific target sequences are provided herein. Additional spacers are well known in the art and can be used with the prime editors utilized in the methods and compositions described herein.

In some embodiments, a PEgRNA comprises three main component elements ordered in the 5′ to 3′ direction, namely: a spacer, a gRNA core, and an extension arm at the 3′ end. In some embodiments, the extension arm may further be divided into the following structural elements in the 5′ to 3′ direction, namely: an edit template, a homology arm, and a primer binding site. In some embodiments, the extension arm may further be divided into the following structural elements in the 5′ to 3′ direction, namely: a homology arm, an edit template, and a primer binding site. In some embodiments, the extension arm may further be divided into the following structural elements in the 5′ to 3′ direction, namely: a DNA synthesis template (e.g., a RT template), and a primer binding site. In addition, the PEgRNA may comprise an optional 3′ end modifier region and an optional 5′ end modifier region. Still further, the PEgRNA may comprise a transcriptional termination signal at the 3′ end of the PERNA. These structural elements are further defined herein. The depiction of the structure of the PEgRNA is not meant to be limiting and embraces variations in the arrangement of the elements. For example, the optional sequence modifiers and could be positioned within or between any of the other regions shown, and not limited to being located at the 3′ and 5′ ends.

### PEgRNA Modifications

The PEgRNAs may also include additional design modifications that may alter the properties and/or characteristics of PEgRNAs, thereby improving the efficacy of prime editing. In various embodiments, these modifications may belong to one or more of a number of different categories, including but not limited to: (1) designs to enable efficient expression of functional PEgRNAs from non-polymerase III (pol III) promoters, which would enable the expression of longer PEgRNAs without burdensome sequence requirements; (2) modifications to the core, Cas9-binding PEgRNA scaffold, which could improve efficacy; (3) modifications to the PEgRNA to improve RT processivity, allowing the insertion of longer sequences at targeted genomic loci; and (4) addition of RNA motifs to the 5′ or 3′ termini of the PEgRNA that improve PEgRNA stability, enhance RT processivity, prevent misfolding of the PEgRNA, or recruit additional factors important for genome editing.

In one embodiment, PEgRNA could be designed with polIII promoters to improve the expression of longer-length PEgRNA with larger extension arms. sgRNAs are typically expressed from the U6 snRNA promoter. This promoter recruits pol III to express the associated RNA and is useful for expression of short RNAs that are retained within the nucleus. However, pol III is not highly processive and is unable to express RNAs longer than a few hundred nucleotides in length at the levels required for efficient genome editing. Additionally, pol III can stall or terminate at stretches of U's, potentially limiting the sequence diversity that could be inserted using a PEgRNA. Other promoters that recruit polymerase II (such as pCMV) or polymerase I (such as the U1 snRNA promoter) have been examined for their ability to express longer sgRNAs. However, these promoters are typically partially transcribed, which would result in extra sequence 5′ of the spacer in the expressed PEgRNA, which has been shown to result in markedly reduced Cas9: sgRNA activity in a site-dependent manner. Additionally, while pol III-transcribed PEgRNAs can simply terminate in a run of 6-7 U's, PEgRNAs transcribed from pol II or pol I would require a different termination signal. Such signals often also result in polyadenylation, which would result in undesired transport of the PEgRNA from the nucleus. Similarly, RNAs expressed from pol II promoters such as pCMV are typically 5′-capped, also resulting in their nuclear export.

In some aspects, the complexes and methods of present disclosure utilize next-generation modified pegRNAs (also referred to herein as “engineered pegRNAs” or “epegRNAs”) with improved properties, including but not limited to, increased stability and cellular lifespan, and improved binding affinity for a napDNAbp. These modified pegRNAs result in improved genome editing as demonstrated by increased editing efficiency at a wide variety of genomic sites. By appending certain nucleic acid structural motifs to the terminus of the extension arm of a pegRNA, including but not limited to, a prequeosin₁-1 riboswitch aptamer (“evopreQ₁-1”) or a variant thereof, a pseudoknot from the MMLV viral genome (“evopreQ₁-1”) or a variant thereof, a modified tRNA used by MMLV RT as a primer for reverse transcription, or a variant thereof, and a G quadruplex, or a variant thereof, a consistent increase in editing activity may be achieved.

In one embodiment, the modified pegRNAs include a nucleic acid moiety at the 3′ end of the pegRNA. Optionally, the 3′ end of the pegRNA is fused to the nucleic acid moiety through a nucleotide linker. In various embodiments, it will be appreciated that a wide variety of nucleotide sequences will work reasonably well for each genomic target site. Linker length can also be variable. In some cases, linkers ranging in length from 3-18 nucleotides will work. In other cases, the linker may be at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 21 nucleotides, at least 22 nucleotides, at least 23 nucleotides, at least 24 nucleotides, at least 25 nucleotides, at least 26 nucleotides, at least 27 nucleotides, at least 28 nucleotides, at least 29 nucleotides, or at least 30 nucleotides.

In general, the nucleic acid moieties that may be used to modify a pegRNA, for example, by attaching it to the 3′ end of a pegRNA, may include any nucleic acid moiety, including, for instance, a nucleic acid molecule comprising or which forms a double-helix moiety, toeloop moiety, hairpin moiety, stem-loop moiety, pseudoknot moiety, aptamer moiety, G quadraplex moiety, tRNA moiety, or a ribozyme moiety. The nucleic acid moiety may be characterized as forming a secondary nucleic acid structure, a tertiary nucleic acid structure, or a quadruple nucleic acid structure. In other words, the nucleic acid moiety may form any two-dimensional or three-dimensional structure known to be formed by such structures. The nucleic acid moiety may be DNA or RNA.

Without restriction, the following are specific examples of nucleotide motifs that may be appended to the terminus of the extension arm of a pegRNA. Thus, in the case of a 3′ extension arm, the nucleotide motif would be coupled, attached, or otherwise linked to the 3′ of the pegRNA, optionally via a linker. In the case of a 5′ extension arm, the nucleotide motif would be coupled, attached, or otherwise linked to the 5′ end of the pegRNA, optionally via a linker.

Mpknot-1 and variants

| | | |
|---|---|---|
| Mpknot1 | GGGTCAGGAGCCCCCCCCCTGAACCCAGGAT | SEQ ID NO: |
| | AACCCTCAAAGTCGGGGGGCAACCC | 418 |
| Mpknot1 3′ | GGGTCAGGAGCCCCCCCCCTGAACCCAGGA | SEQ ID NO: |
| trimmed | TAACCCTCAAAGTCGGGGGGC | 419 |
| Mpknot1 with | GTCAGGGTCAGGAGCCCCCCCCCTGAACCCAGGATA | SEQ ID NO: |
| 5′ extra | ACCCTCAAAGTCGGGGGGCAACCC | 420 |
| Mpknot1 | GGGTCAGGAGCCCCCCCCCTGAACCCAGGAAAACCC | SEQ ID NO: |
| U38A | TCAAAGTCGGGGGGCAACCC | 421 |
| Mpknot1 | GGGTCAGGAGCCCCCCCCCTGCACCCAGGAAAACCC | SEQ ID NO: |
| U38A A29C | TCAAAGTCGGGGGGCAACCC | 422 |
| MMLC A29C | GGGTCAGGAGCCCCCCCCCTGCACCCAGGATAACCCT | SEQ ID NO: |
| | CAAAGTCGGGGGGCAACCC | 423 |
| Mpknot1 with | GTCAGGGTCAGGAGCCCCCCCCCTGAACCCAGGAAA | SEQ ID NO: |
| 5′ extra and | ACCCTCAAAGTCGGGGGGCAACCC | 424 |
| U38A | | |
| Mpknot1 with | GTCAGGGTCAGGAGCCCCCCCCCTGCACCCAGGAAA | SEQ ID NO: |
| 5′ extra and | ACCCTCAAAGTCGGGGGGCAACCC | 425 |
| U38A A29C | | |
| Mpknot1 with | GTCAGGGTCAGGAGCCCCCCCCCTGCACCCAGGATA | SEQ ID NO: |
| 5′ extra and | ACCCTCAAAGTCGGGGGGCAACCC | 426 |
| A29C | | |

G quadruplexes

| | | |
|---|---|---|
| tns1 | Gggctgggatgggaaaggg | SEQ ID NO: 427 |
| stk40 | Gggacagggcagggacaggg | SEQ ID NO: 428 |
| apc2 | Gggtccgggtctgggtctggg | SEQ ID NO: 429 |
| ceacam4 | Gggctctgggtgggccggg | SEQ ID NO: 430 |
| pitpnm3 | Ggggggctgggaaggg | SEQ ID NO: 431 |
| rlf | Gggagggagggctaggg | SEQ ID NO: 432 |
| erc1 | Gggctgggctgggcaggg | SEQ ID NO: 433 |
| ube3c | Gggcagggctgggaggg | SEQ ID NO: 434 |
| taf15 | Gggtgggagggctggg | SEQ ID NO: 435 |
| stard3 | Gggcagggtctgggctggg | SEO ID NO: 436 |
| g2 | Tggtggtggtgg | SEQ ID NO: 437 |

evopreq1 and similar/variant motifs

| evopreq1 | TTGACGCGGTTCTATCTAGTTACGCGTTAAACCAA | SEQ ID NO: |
|---|---|---|
| | CTAGAAA | 438 |
| evopreq1 motif | CGCGAGTCTAGGGGATAACGCGTTAAACTTCCTAGA | SEQ ID NO: |
| 1 | AGGCGGTT | 439 |
| evopreq1 motif | CGCGGATCTAGATTGTAACGCGTTAAACCATCTAGA | SEQ ID NO: |
| 2 | AGGCGGTT | 440 |
| evopreq1 motif | CGCGTCGCTACCGCCCGGCGCGTTAAACACACTAGA | SEQ ID NO: |
| 3 | AGGCGGTT | 441 |
| shorter preq1- | CGCGGTTCTATCTAGTTACGCGTTAAACCAACTAGA | SEQ ID NO: |
| 1 | A | 442 |
| preq1-1 G5C | TTGACGCGCTTCTATCTAGTTACGCGTTAAACCAAC | SEQ ID NO: |
| (mut1) | TAGAAA | 443 |
| preq1-1 G15C | TTGACGCGGTTCTATCTACTTACGCGTTAAACCAAC | SEQ ID NO: |
| (mut2) | TAGAAA | 444 |

Modified tRNA that is used by MMLV RT as a
primer for reverse transcription

GGCGGGGCTCGTTGGTCTAGGGGTATGATTCTCGCTTCGGG
TGCGAGAGGTCCCGGGTTCAAATCCCGGACGAGCCCCGCC
(SEQ ID NO: 445)

Miscellaneous motifs

xrn1-gcgtaacctccatccgagttgcaagagagggaaa
cgcagtctc (SEQ ID NO: 446)
grp1 intron P4P6-
ggaattgcgggaaaggggtcaacagccgttcagtaccaa
gtctcaggggaaactttgagatggccttgcaaagggtat
ggtaataagctgacggacatggtcctaaccacgcagcca
agtcctaagtcaacagatcttctgttgatatggatgcag
ttca
(SEQ ID NO: 447)

As indicated above, these motifs may be coupled, attached, or otherwise joined to a canonical pegRNA via a linker. Exemplary linkers include, but are not limited to:

Linkers for joining motifs to canonical pegRNA

| Genomic | | SEQ ID | Length |
|---|---|---|---|
| locus | Linker sequence | NO: | (nts) |
| HEK3 | TCTCTCTC | — | 8 |
| HEK3 | TCTCTCTCACACACACAC | 448 | 18 |
| RNF2 | TCATCTCT | — | 8 |
| RNF2 | TCATCTCTACACACACAC | 449 | 18 |
| FANCF | CAATCACT | — | 8 |
| FANCF | CAATCACTACACACACAC | 450 | 18 |
| RUNX1 | AACTCTCT | — | 8 |
| RUNX1 | AACTCTCTACACACACAC | 451 | 18 |
| EMX1 | AACAATCT | — | 8 |
| EMX1 | AACAATCTACACACACAC | 452 | 18 |
| DNMT1 | CCTCTTCT | — | 8 |
| DNMT1 | CCTCTTCTACACACACAC | 453 | 18 |

In some embodiments, a linker will be designed and/or selected based on the genomic site being targeted by prime editing and the modified pegRNA.

In various embodiments, it will be appreciated that a wide variety of nucleotide sequences will work reasonably well for each genomic target site. Linker length is also likely to be variable. In some cases, linkers ranging in length from 3-18 nucleotides will work. In other cases, the linker may be at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 21 nucleotides, at least 22 nucleotides, at least 23 nucleotides, at least 24 nucleotides, at least 25 nucleotides, at least 26 nucleotides, at least 27 nucleotides, at least 28 nucleotides, at least 29 nucleotides, or at least 30 nucleotides.

In one embodiment, the linker is 8 nucleotides in length.

The present disclosure also contemplates variants of the above nucleotide motifs and linkers that have at least 70%, at least 75%, at least 80%, at least 85%, at least 90%, at least 91%, at least 92%, at least 93%, at least 94%, at least 95%, at least 96%, at least 97%, at least 98%, at least 99%, or at least 99.9% sequence identity with any of the above motif and linker sequences.

The pegRNAs may also include additional design improvements that may modify the properties and/or characteristics of pegRNAs thereby improving the efficacy of prime editing. In various embodiments, these improvements may belong to one or more of a number of different categories, including, but not limited to: (1) designs to enable efficient expression of functional pegRNAs from non-polymerase III (pol III) promoters, which would enable the expression of longer pegRNAs without burdensome sequence requirements; (2) improvements to the core, Cas9-binding pegRNA scaffold, which could improve efficacy; (3) modifications to the pegRNA to improve RT processivity, allowing the insertion of longer sequences at targeted genomic loci; and (4) addition of RNA motifs to the 5′ or 3′ termini of the pegRNA that improve pegRNA stability, enhance RT processivity, prevent misfolding of the pegRNA, or recruit additional factors important for genome editing.

In one embodiment, pegRNA could be designed with polIII promoters to improve the expression of longer-length pegRNA with larger extension arms. sgRNAs are typically expressed from the U6 snRNA promoter. This promoter recruits pol III to express the associated RNA and is useful for expression of short RNAs that are retained within the nucleus. However, pol III is not highly processive and is unable to express RNAs longer than a few hundred nucleotides in length at the levels required for efficient genome editing. Additionally, pol III can stall or terminate at stretches of U's, potentially limiting the sequence diversity that could be inserted using a pegRNA. Other promoters that recruit polymerase II (such as pCMV) or polymerase I (such as the U1 snRNA promoter) have been examined for their ability to express longer sgRNAs. However, these promoters are typically partially transcribed, which would result in extra sequence 5′ of the spacer in the expressed pegRNA, which has been shown to result in markedly reduced Cas9: sgRNA activity in a site-dependent manner. Additionally, while pol III-transcribed pegRNAs can simply terminate in a run of 6-7 U's, pegRNAs transcribed from pol II or pol I would require a different termination signal. Often such signals also result in polyadenylation, which would result in undesired transport of the pegRNA from the nucleus. Similarly, RNAs expressed from pol II promoters such as pCMV are typically 5′-capped, also resulting in their nuclear export.

Exemplary U6 promoters include, but are not limited to:

U6 promoter:
(SEQ ID NO: 414)
GAGGGCCTATTTCCCATGATTCCTTCATATTTGCATATACGATAC
AAGGCTGTTAGAGAGATAATTAGAATTAATTTGACTGTAAACACA
AAGATATTAGTACAAAATACGTGACGTAGAAAGTAATAATTTCTT
GGGTAGTTTGCAGTTTTAAAATTATGTTTTAAAATGGACTATCAT
ATGCTTACCGTAACTTGAAAGTATTTCGATTTCTTGGCTTTATAT
ATCTTGTGGAAAGGACGAAACACCG
U6v9 promoter:
(SEQ ID NO: 415)
GCCTGAGGCGTGGGGCCGCCTCCCAAAGACTTCTGGGAGGGCGGT
GCGGCTCAGGCTCTGCCCCGCCTCCGGGGCTATTTGCATACGACC
ATTTCCAGTAATTCCCAGCAGCCACCGTAGCTATATTTGGTAGAA
CAACGAGCACTTTCTCAACTCCAGTCAATAACTACGTTAGTTGCA
TTACACATTGGGCTAATATAAATAGAGGTTAAATCTCTAGGTCAT
TTAAGAGAAGTCGGCCTATGTGTACAGACATTTGTTCCAGGGGCT
TTAAATAGCTGGTGGTGGAACTCAATATTCG
U6v7 promoter:
(SEQ ID NO: 416)
AAGTCCGCGGCACGAGAAATCAAAGCCCCGGGGCCTGGGTCCCAC
GCGGGGTCCCTTACCCAGGGTGCCCCGGGCGCTCATTTGCATGTC
CCACCCAACAGGTAAACCTGACAGATCGGTCGCGGCCAGGTACGG
CCTGGCGGTCAGAGCACCAAACTTACGAGCCTTGTGATGAGTTCC
GTTACATGAAATTCTCCTAAAGGCTCCAAGATGGACAGGAAAGCG
CTCGATTAGGTTACCGTAAGGAAAACAAATGAGAAACTCCCGTGC
CTTATAAGACCTGGGGACGGACTTATTTGCG
U6v4 promoter:
(SEQ ID NO: 417)
AAATTGAGTCATCTGACAGAAATTATCTTTGGCAAGGTTTTAGTC
CTAGGGTTACCAGATGGAATACAGGACATCCATTTAAATTTGAAT
TTCAGATAAACAGTTAACACTTCTCAAGGATAAATATGCCTCAAA
TATTGCACGGGACATATTTATACTAAAAAAAAAGTGTTTTTTTTT
TTCCTGCGATTCAAACTTAACTGGTGTCCTGCATTTGTATTTGTT
AAATCTGTCAATCCTATCTCAGTTTCCTTTGATGGAATGTACCTC
TGTGCTAATATTTAAAAATAGGTTACATTTG

One of ordinary skill in the art will appreciate that these promoter sequences can be trimmed at the 5′ and still function at the same or nearly the same level. For example, any of the U6 promoters could be trimmed at the 5′ end by removing up to 1, 10, 20, 30, 40, 50, 60, 70, 80, 90, or 100 nucleotides from the 5′ end, i.e., approximately 30% of the promoter length. In other embodiments, up to 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% of the length of the promoter may be trimmed from the 5′ end.

One of ordinary skill in the art will also appreciate that other promoters could be used to improve the expression of longer length pegRNAs with larger extension arms. For example, in different cell types, other promoters may be preferred and result in greater expression of the longer length pegRNAs.

Previously, Rinn and coworkers screened a variety of expression platforms for the production of long-noncoding RNA-(lncRNA) tagged sgRNAs. These platforms include RNAs expressed from pCMV and that terminate in the ENE element from the MALAT1 ncRNA from humans, the PAN ENE element from KSHV, or the 3′ box from U1 snRNA. Notably, the MALAT1 ncRNA and PAN ENEs form triple helices protecting the poly A-tail. These constructs could also enhance RNA stability. It is contemplated that these expression systems will also allow the expression of longer PEgRNAs.

In addition, a series of methods have been designed for the cleavage of the portion of the pol II promoter that would be transcribed as part of the PEgRNA, adding either a self-cleaving ribozyme such as the hammerhead, pistol, hatchet, hairpin, VS, twister, or twister sister ribozymes, or other self-cleaving elements to process the transcribed guide, or a hairpin that is recognized by Csy4 and also leads to processing of the guide. Also, it is hypothesized that incorporation of multiple ENE motifs could lead to improved PERNA expression and stability, as previously demonstrated for the KSHV PAN RNA and element. It is also anticipated that circularizing the PEgRNA in the form of a circular intronic RNA (ciRNA) could also lead to enhanced RNA expression and stability, as well as nuclear localization.

In various embodiments, the PEgRNA may include various above elements, as exemplified by the following sequences.

Non-limiting example 1—PEgRNA expression platform consisting of pCMV, Csy4 hairpin, the PEgRNA, and MALATI ENE

(SEQ ID NO: 147)
TAGTTATTAATAGTAATCAATTACGGGGTCATTAGTTCATAGCCCATATA
TGGAGTTCCGCGTTACATAACTTACGGTAAATGGCCCGCCTGGCTGACCG
CCCAACGACCCCCGCCCATTGACGTCAATAATGACGTATGTTCCCATAGT
AACGCCAATAGGGACTTTCCATTGACGTCAATGGGTGGAGTATTTACGGT
AAACTGCCCACTTGGCAGTACATCAAGTGTATCATATGCCAAGTACGCCC
CCTATTGACGTCAATGACGGTAAATGGCCCGCCTGGCATTATGCCCAGTA
CATGACCTTATGGGACTTTCCTACTTGGCAGTACATCTACGTATTAGTCA
TCGCTATTACCATGGTGATGCGGTTTTGGCAGTACATCAATGGGCGTGGA
TAGCGGTTTGACTCACGGGGATTTCCAAGTCTCCACCCCATTGACGTCAA
TGGGAGTTTGTTTTGGCACCAAAATCAACGGGACTTTCCAAAATGTCGTA
ACAACTCCGCCCCATTGACGCAAATGGGCGGTAGGCGTGTACGGTGGGAG
GTCTATATAAGCAGAGCTGGTTTAGTGAACCGTCAGATCGTTCACTGCCG
TATAGGCAGGGCCCAGACTGAGCACGTGAGTTTTAGAGCTAGAAATAGCA
AGTTAAAATAAGGCTAGTCCGTTATCAACTTGAAAAAGTGGGACCGAGTC
GGTCCTCTGCCATCAAAGCGTGCTCAGTCTGTTTTAGGGTCATGAAGGTT
TTTCTTTTCCTGAGAAAACAACACGTATTGTTTTCTCAGGTTTTGCTTTT
TGGCCTTTTTCTAGCTTAAAAAAAAAAAAAGCAAAAGATGCTGGTGGTTG
GCACTCCTGGTTTCCAGGACGGGGTTCAAATCCCTGCGGCGTCTTTGCTT
TGACT

Non-limiting example 2—PERNA expression platform consisting of pCMV, Csy4 hairpin, the PERNA, and PAN ENE

(SEQ ID NO: 148)
TAGTTATTAATAGTAATCAATTACGGGGTCATTAGTTCATAGCCCATATA
TGGAGTTCCGCGTTACATAACTTACGGTAAATGGCCCGCCTGGCTGACCG
CCCAACGACCCCCGCCCATTGACGTCAATAATGACGTATGTTCCCATAGT
AACGCCAATAGGGACTTTCCATTGACGTCAATGGGTGGAGTATTTACGGT
AAACTGCCCACTTGGCAGTACATCAAGTGTATCATATGCCAAGTACGCCC
CCTATTGACGTCAATGACGGTAAATGGCCCGCCTGGCATTATGCCCAGTA
CATGACCTTATGGGACTTTCCTACTTGGCAGTACATCTACGTATTAGTCA
TCGCTATTACCATGGTGATGCGGTTTTGGCAGTACATCAATGGGCGTGGA
TAGCGGTTTGACTCACGGGGATTTCCAAGTCTCCACCCCATTGACGTCAA
TGGGAGTTTGTTTTGGCACCAAAATCAACGGGACTTTCCAAAATGTCGTA
ACAACTCCGCCCCATTGACGCAAATGGGCGGTAGGCGTGTACGGTGGGAG
GTCTATATAAGCAGAGCTGGTTTAGTGAACCGTCAGATCGTTCACTGCCG
TATAGGCAGGGCCCAGACTGAGCACGTGAGTTTTAGAGCTAGAAATAGCA
AGTTAAAATAAGGCTAGTCCGTTATCAACTTGAAAAAGTGGGACCGAGTC
GGTCCTCTGCCATCAAAGCGTGCTCAGTCTGTTTTGTTTTGGCTGGGTTT
TTCCTTGTTCGCACCGGACACCTCCAGTGACCAGACGGCAAGGTTTTTAT
CCCAGTGTATATTGGAAAAACATGTTATACTTTTGACAATTTAACGTGCC
TAGAGCTCAAATTAAACTAATACCATAACGTAATGCAACTTACAACATAA
ATAAAGGTCAATGTTTAATCCATAAAAAAAAAAAAAAAAAAA

Non-limiting example 3—PEgRNA expression platform consisting of pCMV, Csy4 hairpin, the PERNA, and 3xPAN ENE

(SEQ ID NO: 149)
TAGTTATTAATAGTAATCAATTACGGGGTCATTAGTTCATAGCCCATATA
TGGAGTTCCGCGTTACATAACTTACGGTAAATGGCCCGCCTGGCTGACCG
CCCAACGACCCCCGCCCATTGACGTCAATAATGACGTATGTTCCCATAGT
AACGCCAATAGGGACTTTCCATTGACGTCAATGGGTGGAGTATTTACGGT
AAACTGCCCACTTGGCAGTACATCAAGTGTATCATATGCCAAGTACGCCC
CCTATTGACGTCAATGACGGTAAATGGCCCGCCTGGCATTATGCCCAGTA
CATGACCTTATGGGACTTTCCTACTTGGCAGTACATCTACGTATTAGTCA
TCGCTATTACCATGGTGATGCGGTTTTGGCAGTACATCAATGGGCGTGGA
TAGCGGTTTGACTCACGGGGATTTCCAAGTCTCCACCCCATTGACGTCAA
TGGGAGTTTGTTTTGGCACCAAAATCAACGGGACTTTCCAAAATGTCGTA
ACAACTCCGCCCCATTGACGCAAATGGGCGGTAGGCGTGTACGGTGGGAG
GTCTATATAAGCAGAGCTGGTTTAGTGAACCGTCAGATCGTTCACTGCCG
TATAGGCAGGGCCCAGACTGAGCACGTGAGTTTTAGAGCTAGAAATAGCA
AGTTAAAATAAGGCTAGTCCGTTATCAACTTGAAAAAGTGGGACCGAGTC
GGTCCTCTGCCATCAAAGCGTGCTCAGTCTGTTTTGTTTTGGCTGGGTTT
TTCCTTGTTCGCACCGGACACCTCCAGTGACCAGACGGCAAGGTTTTTAT
CCCAGTGTATATTGGAAAAACATGTTATACTTTTGACAATTTAACGTGCC
TAGAGCTCAAATTAAACTAATACCATAACGTAATGCAACTTACAACATAA
ATAAAGGTCAATGTTTAATCCATAAAAAAAAAAAAAAAAAAAACACACTG
TTTTGGCTGGGTTTTTCCTTGTTCGCACCGGACACCTCCAGTGACCAGAC
GGCAAGGTTTTTATCCCAGTGTATATTGGAAAAACATGTTATACTTTTGA
CAATTTAACGTGCCTAGAGCTCAAATTAAACTAATACCATAACGTAATGC
AACTTACAACATAAATAAAGGTCAATGTTTAATCCATAAAAAAAAAAAAA
AAAAAATCTCTCTGTTTTGGCTGGGTTTTTCCTTGTTCGCACCGGACACC
TCCAGTGACCAGACGGCAAGGTTTTTATCCCAGTGTATATTGGAAAAACA
TGTTATACTTTTGACAATTTAACGTGCCTAGAGCTCAAATTAAACTAATA
CCATAACGTAATGCAACTTACAACATAAATAAAGGTCAATGTTTAATCCA
TAAAAAAAAAAAAAAAAAAA

Non-limiting example 4—PEgRNA expression platform consisting of pCMV, Csy4 hairpin, the PERNA, and 3′ box

(SEQ ID NO: 150)
TAGTTATTAATAGTAATCAATTACGGGGTCATTAGTTCATAGCCCATATA
TGGAGTTCCGCGTTACATAACTTACGGTAAATGGCCCGCCTGGCTGACCG
CCCAACGACCCCCGCCCATTGACGTCAATAATGACGTATGTTCCCATAGT
AACGCCAATAGGGACTTTCCATTGACGTCAATGGGTGGAGTATTTACGGT
AAACTGCCCACTTGGCAGTACATCAAGTGTATCATATGCCAAGTACGCCC
CCTATTGACGTCAATGACGGTAAATGGCCCGCCTGGCATTATGCCCAGTA
CATGACCTTATGGGACTTTCCTACTTGGCAGTACATCTACGTATTAGTCA
TCGCTATTACCATGGTGATGCGGTTTTGGCAGTACATCAATGGGCGTGGA
TAGCGGTTTGACTCACGGGGATTTCCAAGTCTCCACCCCATTGACGTCAA
TGGGAGTTTGTTTTGGCACCAAAATCAACGGGACTTTCCAAAATGTCGTA
ACAACTCCGCCCCATTGACGCAAATGGGCGGTAGGCGTGTACGGTGGGAG
GTCTATATAAGCAGAGCTGGTTTAGTGAACCGTCAGATCGTTCACTGCCG
TATAGGCAGGGCCCAGACTGAGCACGTGAGTTTTAGAGCTAGAAATAGCA
AGTTAAAATAAGGCTAGTCCGTTATCAACTTGAAAAAGTGGGACCGAGTC
GGTCCTCTGCCATCAAAGCGTGCTCAGTCTGTTTGTTTCAAAAGTAGACT
GTACGCTAAGGGTCATATCTTTTTTTGTTTGGTTTGTGTCTTGGTTGGCG
TCTTAAA

Non-limiting example 5—PEgRNA expression platform consisting of pU1, Csy4 hairpin, the PEgRNA, and 3′ box

(SEQ ID NO: 151)
CTAAGGACCAGCTTCTTTGGGAGAGAACAGACGCAGGGGCGGGAGGGAAA
AAGGGAGAGGCAGACGTCACTTCCCCTTGGCGGCTCTGGCAGCAGATTGG
TCGGTTGAGTGGCAGAAAGGCAGACGGGGACTGGGCAAGGCACTGTCGGT
GACATCACGGACAGGGCGACTTCTATGTAGATGAGGCAGCGCAGAGGCTG
CTGCTTCGCCACTTGCTGCTTCACCACGAAGGAGTTCCCGTGCCCTGGGA
GCGGGTTCAGGACCGCTGATCGGAAGTGAGAATCCCAGCTGTGTGTCAGG
GCTGGAAAGGGCTCGGGAGTGCGCGGGGCAAGTGACCGTGTGTGTAAAGA
GTGAGGCGTATGAGGCTGTGTCGGGGCAGAGGCCCAAGATCTCAGTTCAC
TGCCGTATAGGCAGGGCCCAGACTGAGCACGTGAGTTTTAGAGCTAGAAA
TAGCAAGTTAAAATAAGGCTAGTCCGTTATCAACTTGAAAAAGTGGGACC
GAGTCGGTCCTCTGCCATCAAAGCGTGCTCAGTCTGTTTCAGCAAGTTCA
GAGAAATCTGAACTTGCTGGATTTTTGGAGCAGGGAGATGGAATAGGAGC
TTGCTCCGTCCACTCCACGCATCGACCTGGTATTGCAGTACCTCCAGGAA
CGGTGCACCCACTTTCTGGAGTTTCAAAAGTAGACTGTACGCTAAGGGTC
ATATCTTTTTTTGTTTGGTTTGTGTCTTGGTTGGCGTCTTAAA

In various other embodiments, the PERNA may be improved by introducing modifications to the scaffold or core sequences. The core, Cas9-binding PEgRNA scaffold can likely be improved to enhance PE activity. Several such approaches have already been demonstrated. For instance, the first pairing element of the scaffold (P1) contains a GTTTT-AAAAC (SEQ ID NO: 146) pairing element. Such runs of Ts have been shown to result in pol III pausing and premature termination of the RNA transcript. Rational mutation of one of the T-A pairs to a G-C pair in this portion of P1 has been shown to enhance sgRNA activity, suggesting this approach would also be feasible for PEgRNAs. Additionally, increasing the length of P1 has also been shown to enhance sgRNA folding and lead to improved activity, suggesting it as another avenue for the modification of PEgRNA activity. Example modifications to the core can include:

PEgRNA containing a 6 nt extension to P1
(SEQ ID NO: 152)
GGCCCAGACTGAGCACGTGAGTTTTAGAGCTAGCTCATGAAAATGA
GCTAGCAAGTTAAAATAAGGCTAGTCCGTTATCAACTTGAAAAAGT
GGGACCGAGTCGGTCCTCTGCCATCAAAGCGTGCTCAGTCTGTTTT
TTT
PEgRNA containing a T-A to G-C mutation
within P1
(SEQ ID NO: 153)
GGCCCAGACTGAGCACGTGAGTTTGAGAGCTAGAAATAGCAAGTTTA
AATAAGGCTAGTCCGTTATCAACTTGAAAAAGTGGGACCGAGTCGGT
CCTCTGCCATCAAAGCGTGCTCAGTCTGTTTTTTT

In various other embodiments, the PERNA may be modified at the edit template region. As the size of the insertion templated by the PEgRNA increases, it is more likely to be degraded by endonucleases, undergo spontaneous hydrolysis, or fold into secondary structures unable to be reverse-transcribed by the RT, or that disrupt folding of the PEgRNA scaffold and subsequent Cas9-RT binding. Accordingly, it is likely that modification to the template of the PEgRNA might be necessary to affect large insertions, such as the insertion of whole genes. Some strategies to do so include the incorporation of modified nucleotides within a synthetic or semi-synthetic PEgRNA that render the RNA more resistant to degradation or hydrolysis or less likely to adopt inhibitory secondary structures. Such modifications could include 8-aza-7-deazaguanosine, which would reduce RNA secondary structure in G-rich sequences; locked-nucleic acids (LNA) that reduce degradation and enhance certain kinds of RNA secondary structure; 2′-O-methyl, 2′-fluoro, or 2′-O-methoxyethoxy modifications that enhance RNA stability. Such modifications could also be included elsewhere in the PEgRNA to enhance stability and activity. Alternatively, or additionally, the template of the PEgRNA could be designed such that it is also more likely to adopt simple secondary structures that are able to allow processing by the RT. Such simple structures would act as a thermodynamic sink, making it less likely that more complicated structures that would prevent reverse transcription would occur. Finally, one could also split the template into two separate PEgRNAs. In such a design, a prime editors, e.g., a nCas9-RT fusion protein, would be used to initiate transcription, and also to recruit a separate template RNA to the targeted site via an RNA-binding protein fused to Cas9 or an RNA recognition element on the PEgRNA itself such as the MS2 aptamer. The RT could either directly bind to this separate template RNA, or initiate reverse transcription on the original PEgRNA before swapping to the second template. Such an approach could allow long insertions by both preventing misfolding of the PEgRNA upon addition of the long template, and also by not requiring dissociation of Cas9 from the genome for long insertions to occur, which could possibly inhibit PE-based long insertions.

In still other embodiments, the PEgRNA may be modified by introducing additional RNA motifs at the 5′ and 3′ termini of the PEgRNAs, or even at positions therein between (e.g., in the gRNA core region, or the spacer). Several such motifs-such as the PAN ENE from KSHV and the ENE from MALATI were discussed above as possible means to terminate expression of longer PEgRNAs from non-pol III promoters. These elements form RNA triple helices that engulf the polyA tail, resulting in their being retained within the nucleus. However, by forming complex structures at the 3′ terminus of the PEgRNA that occlude the terminal nucleotide, these structures would also likely help prevent exonuclease-mediated degradation of PEgRNAs.

Other structural elements inserted at the 3′ terminus could also enhance RNA stability, albeit without allowing for termination from non-pol III promoters. Such motifs could include hairpins or RNA quadruplexes that would occlude the 3′ terminus, or self-cleaving ribozymes such as HDV that would result in the formation of a 2′-3′-cyclic phosphate at the 3′ terminus, and also potentially render the PEgRNA less likely to be degraded by exonucleases. Inducing the PERNA to cyclize via incomplete splicing (to form a ciRNA) could also increase PEgRNA stability and result in the PEgRNA being retained within the nucleus.

Additional RNA motifs could also improve RT processivity or enhance PEgRNA activity by enhancing RT binding to the DNA-RNA duplex. Addition of the native sequence bound by the RT in its cognate retroviral genome could enhance RT activity. This could include the native primer binding site (PBS), polypurine tract (PPT), or kissing loops involved in retroviral genome dimerization and initiation of transcription.

Addition of dimerization motifs, such as kissing loops or a GNRA tetraloop/tetraloop receptor pair, at the 5′ and 3′ termini of the PERNA could also result in effective circularization of the PEgRNA, improving stability. Additionally, it is envisioned that addition of these motifs could allow the physical separation of the PEgRNA spacer and primer, preventing occlusion of the spacer, which would hinder PE activity. Short 5′ extensions or 3′ extensions to the PEgRNA that form a small toehold hairpin in the spacer region or along the primer binding site could also compete favorably against the annealing of intracomplementary regions along the length of the PEgRNA, e.g., the interaction between the spacer and the primer binding site that can occur. Finally, kissing loops could also be used to recruit other template RNAs to the genomic site and enable swapping of RT activity from one RNA to the other. A number of secondary RNA structures may be engineered into any region of the PERNA, including in the terminal portions of the extension arm (i.e., e1 and e2), as shown.

Example modifications include, but are not limited to:

PEgRNA-HDV fusion
(SEQ ID NO: 154)
GGCCCAGACTGAGCACGTGAGTTTTAGAGCTAGAAATAGCAAGTTAAAATAAGGC
TAGTCCGTTATCAACTTGAAAAAGTGGGACCGAGTCGGTCCTCTGCCATCAAAGC
GTGCTCAGTCTGGGCCGGCATGGTCCCAGCCTCCTCGCTGGCGCCGGCTGGGCAA
CATGCTTCGGCATGGCGAATGGGACTTTTTTT
PEgRNA-MMLV kissing loop
(SEQ ID NO: 155)
GGTGGGAGACGTCCCACCGGCCCAGACTGAGCACGTGAGTTTTAGAGCTAGAAA
TAGCAAGTTAAAATAAGGCTAGTCCGTTATCAACTTGAAAAAGTGGGACCGAGTC
GGTCCTCTGCCATCAAAGCTTCGACCGTGCTCAGTCTGGTGGGAGACGTCCCACC
TTTTTTT
PEgRNA-VS ribozyme kissing loop
(SEQ ID NO: 156)
GAGCAGCATGGCGTCGCTGCTCACGGCCCAGACTGAGCACGTGAGTTTTAGAGCT
AGAAATAGCAAGTTAAAATAAGGCTAGTCCGTTATCAACTTGAAAAAGTGGGACC
GAGTCGGTCCTCTGCCATCAAAGCTTCGACCGTGCTCAGTCTCCATCAGTTGACA
CCCTGAGGTTTTTTT
PEgRNA-GNRA tetraloop/tetraloop receptor
(SEQ ID NO: 157)
GCAGACCTAAGTGGUGACATATGGTCTGGGCCCAGACTGAGCACGTGAGTTTTAG
AGCTAUACGTAGCAAGTTAAAATAAGGCTAGTCCGTTATCAACTTUACGAAGTGG
GACCGAGTCGGTCCTCTGCCATCAAAGCTTCGACCGTGCTCAGTCTGCATGCGATT
AGAAATAATCGCATGTTTTTTT
PEgRNA template switching secondary RNA-HDV fusion
(SEQ ID NO: 158)
TCTGCCATCAAAGCTGCGACCGTGCTCAGTCTGGTGGGAGACGTCCCACCGGCCG
GCATGGTCCCAGCCTCCTCGCTGGCGCCGGCTGGGCAACATGCTTCGGCATGGCG
AATGGGACTTTTTTT

PEgRNA scaffolds can be further improved via directed evolution, in an analogous fashion to how SpCas9 and prime editors (PE) have been improved. Directed evolution can enhance PEgRNA recognition by Cas9 or evolved Cas9 variants. Additionally, different PEgRNA scaffold sequences are likely optimal at different genomic loci, either enhancing PE activity at the site in question, reducing off-target activities, or both. Finally, evolution of PEgRNA scaffolds to which other RNA motifs have been added would almost certainly improve the activity of the fused PEgRNA relative to the unevolved, fusion RNA. For instance, evolution of allosteric ribozymes composed of c-di-GMP-I aptamers and hammerhead ribozymes led to dramatically improved activity, suggesting that evolution would improve the activity of hammerhead-PERNA fusions as well. In addition, while Cas9 currently does not generally tolerate 5′ extension of the sgRNA as well as tolerating 3′ extensions, directed evolution will likely generate mutations that mitigate this intolerance, allowing additional RNA motifs to be utilized.

In various embodiments, other scaffolds that have been shown to improve activity relative to canonical sgRNA scaffolds may be used in pegRNAs and epegRNAs as described herein. Such improvements may include, for example, those disclosed in Chen, B. et al., Dynamic Imaging of Genomic Loci in Living Human Cells by an Optimized CRISPR/Cas System. Cell. 2013, 155(7), 1479-1471 and Jost, M. et al., Titrating expression using libraries of systematically attenuated CRISPR guide RNAs. Nat. Biotechnol. 2020, 38, 355-364, which are herein incorporated by reference in their entirety. These improvements may enhance epegRNA activity through improved binding to the prime editor and/or improved expression. Stabilization of the sgRNA scaffold could also reduce PBS/spacer interactions that inhibit pegRNA and epegRNA activity.

Exemplary epegRNAs incorporating improved sgRNA scaffolds include, but are not limited to:

HEK3 1-15del standard scaffold evopreQ1
(SEQ ID NO: 256)
GGCCCAGACTGAGCACGTGAGTTTTAGAGCTAGAAATAGCAAGTTAAAATAAGG
CTAGTCCGTTATCAACTTGAAAAAGTGGCACCGAGTCGGTGCGCCCTCTGGAGG
AAGCAGGGCTTCCCGTGCTCAGTCTGTCTCTCTCTTGACGCGGTTCTATCTAGTTA
CGCGTTAAACCAACTAGAAATTTTTT
HEK3 1-15del cr748 evopreQ1
(SEQ ID NO: 257)
GGCCCAGACTGAGCACGTGAGTTTAAGAGCTAAGCTGGAAACAGCATAGCAAGT
TTAAATAAGGCTAGTCCGTTATCAACTCGAGAGAGTGGCACCGAGTCGGTGCTG
CCCTCTGGAGGAAGCAGGGCTTCCCGTGCTCAGTCTGTCTCTCTCTTGACGCGGT
TCTATCTAGTTACGCGTTAAACCAACTAGAAATTTTTT
HEK3 1-15del cr289 evopreQ1
(SEQ ID NO: 258)
GGCCCAGACTGAGCACGTGAGTTTAAGAGCTAAGCTGGAAACAGCATAGCAAGT
TTAAATAAGGCTAGTCCGTTATCAACTGGAAACAGTGGCACCGAGTCGGTGCTG
CCCTCTGGAGGAAGCAGGGCTTCCCGTGCTCAGTCTGTCTCTCTCTTGACGCGGT
TCTATCTAGTTACGCGTTAAACCAACTAGAAATTTTTT
HEK3 1-15del cr622 evopreQ1
(SEQ ID NO: 259)
GGCCCAGACTGAGCACGTGAGTTTAAGAGCTAAGCTGGAAACAGCATAGCAAGT
TTAAATAAGGCTAGTCCGTTATCAGCTGGAAACAGCGGCACCGAGTCGGTGCTG
CCCTCTGGAGGAAGCAGGGCTTCCCGTGCTCAGTCTGTCTCTCTCTTGACGCGGT
TCTATCTAGTTACGCGTTAAACCAACTAGAAATTTTTT
HEK3 1-15del cr772 evopreQ1
(SEQ ID NO: 260)
GGCCCAGACTGAGCACGTGAGTTTAAGAGCTAAGCTGGAAACAGCATAGCAAGT
TTAAATAAGGCTAGTCCGTTATCAACTCGAAAGAGTGGCACCGAGTCGGTGCTG
CCCTCTGGAGGAAGCAGGGCTTCCCGTGCTCAGTCTGTCTCTCTCTTGACGCGGT
TCTATCTAGTTACGCGTTAAACCAACTAGAAATTTTTT
HEK3 1-15del cr532 evopreQ1
(SEQ ID NO: 261)
GGCCCAGACTGAGCACGTGAGTTTAAGAGCTAAGCTGGAAACAGCATAGCAAGT
TTAAATAAGGCTAGTCCGTTATCAACGTGAAAACGTGACTCCGAGTCGGAGTTGC
CCTCTGGAGGAAGCAGGGCTTCCCGTGCTCAGTCTGTCTCTCTCTTGACGCGGTT
CTATCTAGTTACGCGTTAAACCAACTAGAAATTTTTT
HEK3 1-15del cr961 evopreQ1
(SEQ ID NO: 262)
GGCCCAGACTGAGCACGTGAGTTTAAGAGCTAAGCTGGAAACAGCATAGCAAGT
TTAAATAAGGCTAGTCCGTTATCAACTCGAAAGAGTGCAACCGAGTCGGTTGTGC
CCTCTGGAGGAAGCAGGGCTTCCCGTGCTCAGTCTGTCTCTCTCTTGACGCGGTT
CTATCTAGTTACGCGTTAAACCAACTAGAAATTTTTT
HEK3 1-15del flip and extension scaffold evopreQ1
(SEQ ID NO: 263)
GGCCCAGACTGAGCACGTGAGTTTAAGAGCTAAGCTGGAAACAGCATAGCAAGT
TTAAATAAGGCTAGTCCGTTATCAACTTGAAAAAGTGGCACCGAGTCGGTGCTGC
CCTCTGGAGGAAGCAGGGCTTCCCGTGCTCAGTCTGTCTCTCTCTTGACGCGGTT
CTATCTAGTTACGCGTTAAACCAACTAGAAATTTTTT
RNF2 1-15del cr748 evopreQ1
(SEQ ID NO: 264)
GTCATCTTAGTCATTACCTGGTTTAAGAGCTAAGCTGGAAACAGCATAGCAAGTT
TAAATAAGGCTAGTCCGTTATCAACTCGAGAGAGTGGCACCGAGTCGGTGCTAT
GGGAACTCAGTTTATATGAGTTAGTAATGACTAAGATGTCATCTCTTTGACGCGG
TTCTATCTAGTTACGCGTTAAACCAACTAGAAATTTTTT
RNF2 1-15del cr289 evopreQ1
(SEQ ID NO: 265)
GTCATCTTAGTCATTACCTGGTTTAAGAGCTAAGCTGGAAACAGCATAGCAAGTT
TAAATAAGGCTAGTCCGTTATCAACTGGAAACAGTGGCACCGAGTCGGTGCTAT
GGGAACTCAGTTTATATGAGTTAGTAATGACTAAGATGTCATCTCTTTGACGCGG
TTCTATCTAGTTACGCGTTAAACCAACTAGAAATTTTTT
RNF2 1-15del cr622 evopreQ1
(SEQ ID NO: 266)
GTCATCTTAGTCATTACCTGGTTTAAGAGCTAAGCTGGAAACAGCATAGCAAGTT
TAAATAAGGCTAGTCCGTTATCAGCTGGAAACAGCGGCACCGAGTCGGTGCTAT
GGGAACTCAGTTTATATGAGTTAGTAATGACTAAGATGTCATCTCTTTGACGCGG
TTCTATCTAGTTACGCGTTAAACCAACTAGAAATTTTTT
RNF2 1-15del cr772 evopreQ1
(SEQ ID NO: 267)
GTCATCTTAGTCATTACCTGGTTTAAGAGCTAAGCTGGAAACAGCATAGCAAGTT
TAAATAAGGCTAGTCCGTTATCAACTCGAAAGAGTGGCACCGAGTCGGTGCTAT
GGGAACTCAGTTTATATGAGTTAGTAATGACTAAGATGTCATCTCTTTGACGCGG
TTCTATCTAGTTACGCGTTAAACCAACTAGAAATTTTTT
RNF2 1-15del cr532 evopreQ1
(SEQ ID NO: 268)
GTCATCTTAGTCATTACCTGGTTTAAGAGCTAAGCTGGAAACAGCATAGCAAGTT
TAAATAAGGCTAGTCCGTTATCAACGTGAAAACGTGACTCCGAGTCGGAGTTAT
GGGAACTCAGTTTATATGAGTTAGTAATGACTAAGATGTCATCTCTTTGACGCGG
TTCTATCTAGTTACGCGTTAAACCAACTAGAAATTTTTT
RNF2 1-15del cr961 evopreQ1
(SEQ ID NO: 269)
GTCATCTTAGTCATTACCTGGTTTAAGAGCTAAGCTGGAAACAGCATAGCAAGTT
TAAATAAGGCTAGTCCGTTATCAACTCGAAAGAGTGCAACCGAGTCGGTTGTAT
GGGAACTCAGTTTATATGAGTTAGTAATGACTAAGATGTCATCTCTTTGACGCGG
TTCTATCTAGTTACGCGTTAAACCAACTAGAAATTTTTT
RNF2 1-15del flip and extension scaffold evopreQ1
(SEQ ID NO: 270)
GTCATCTTAGTCATTACCTGGTTTAAGAGCTAAGCTGGAAACAGCATAGCAAGTT
TAAATAAGGCTAGTCCGTTATCAACTTGAAAAAGTGGCACCGAGTCGGTGCTAT
GGGAACTCAGTTTATATGAGTTAGTAATGACTAAGATGTCATCTCTTTGACGCGG
TTCTATCTAGTTACGCGTTAAACCAACTAGAAATTTTTT
RUNX1 1-15del standard scaffold evopreQ1
(SEQ ID NO: 271)
GCATTTTCAGGAGGAAGCGAGTTTTAGAGCTAGAAATAGCAAGTTAAAATAAGG
CTAGTCCGTTATCAACTTGAAAAAGTGGCACCGAGTCGGTGCTACGAAGGAAAT
GACTCAAATATGCCTTCCTCCTGAAAATAACTCTCTTTGACGCGGTTCTATCTAGT
TACGCGTTAAACCAACTAGAAATTTTTT
RUNX1 1-15del cr748 evopreQ1
(SEQ ID NO: 272)
GCATTTTCAGGAGGAAGCGAGTTTAAGAGCTAAGCTGGAAACAGCATAGCAAGT
TTAAATAAGGCTAGTCCGTTATCAACTCGAGAGAGTGGCACCGAGTCGGTGCTTA
CGAAGGAAATGACTCAAATATGCCTTCCTCCTGAAAATAACTCTCTTTGACGCGG
TTCTATCTAGTTACGCGTTAAACCAACTAGAAATTTTTT
RUNX1 1-15del cr289 evopreQ1
(SEQ ID NO: 273)
GCATTTTCAGGAGGAAGCGAGTTTAAGAGCTAAGCTGGAAACAGCATAGCAAGT
TTAAATAAGGCTAGTCCGTTATCAACTGGAAACAGTGGCACCGAGTCGGTGCTTA
CGAAGGAAATGACTCAAATATGCCTTCCTCCTGAAAATAACTCTCTTTGACGCGG
TTCTATCTAGTTACGCGTTAAACCAACTAGAAATTTTTT
RUNX1 1-15del cr622 evopreQ1
(SEQ ID NO: 274)
GCATTTTCAGGAGGAAGCGAGTTTAAGAGCTAAGCTGGAAACAGCATAGCAAGT
TTAAATAAGGCTAGTCCGTTATCAGCTGGAAACAGCGGCACCGAGTCGGTGCTT
ACGAAGGAAATGACTCAAATATGCCTTCCTCCTGAAAATAACTCTCTTTGACGCG
GTTCTATCTAGTTACGCGTTAAACCAACTAGAAATTTTTT
RUNX1 1-15del cr772 evopreQ1
(SEQ ID NO: 275)
GCATTTTCAGGAGGAAGCGAGTTTAAGAGCTAAGCTGGAAACAGCATAGCAAGT
TTAAATAAGGCTAGTCCGTTATCAACTCGAAAGAGTGGCACCGAGTCGGTGCTTA
CGAAGGAAATGACTCAAATATGCCTTCCTCCTGAAAATAACTCTCTTTGACGCGG
TTCTATCTAGTTACGCGTTAAACCAACTAGAAATTTTTT
RUNX1 1-15del cr532 evopreQ1
(SEQ ID NO: 276)
GCATTTTCAGGAGGAAGCGAGTTTAAGAGCTAAGCTGGAAACAGCATAGCAAGT
TTAAATAAGGCTAGTCCGTTATCAACGTGAAAACGTGACTCCGAGTCGGAGTTTA
CGAAGGAAATGACTCAAATATGCCTTCCTCCTGAAAATAACTCTCTTTGACGCGG
TTCTATCTAGTTACGCGTTAAACCAACTAGAAATTTTTT
RUNX1 1-15del cr961 evopreQ1
(SEQ ID NO: 277)
GCATTTTCAGGAGGAAGCGAGTTTAAGAGCTAAGCTGGAAACAGCATAGCAAGT
TTAAATAAGGCTAGTCCGTTATCAACTCGAAAGAGTGCAACCGAGTCGGTTGTTA
CGAAGGAAATGACTCAAATATGCCTTCCTCCTGAAAATAACTCTCTTTGACGCGG
TTCTATCTAGTTACGCGTTAAACCAACTAGAAATTTTTT
RUNX1 1-15del flip and extension scaffold evopreQ1
(SEQ ID NO: 278)
GCATTTTCAGGAGGAAGCGAGTTTAAGAGCTAAGCTGGAAACAGCATAGCAAGT
TTAAATAAGGCTAGTCCGTTATCAACTTGAAAAAGTGGCACCGAGTCGGTGCTTA
CGAAGGAAATGACTCAAATATGCCTTCCTCCTGAAAATAACTCTCTTTGACGCGG
TTCTATCTAGTTACGCGTTAAACCAACTAGAAATTTTTT
RUNX1 +5G-T standard scaffold evopreQ1
(SEQ ID NO: 279)
GCATTTTCAGGAGGAAGCGAGTTTTAGAGCTAGAAATAGCAAGTTAAAATAAGG
CTAGTCCGTTATCAACTTGAAAAAGTGGCACCGAGTCGGTGCTGTCTGAAGCAAT
CGCTTCCTCCTGAAAATAACTCTCTTTGACGCGGTTCTATCTAGTTACGCGTTAAA
CCAACTAGAAATTTTTT
RUNX1 +5G-T cr748 evopreQ1
(SEQ ID NO: 280)
GCATTTTCAGGAGGAAGCGAGTTTAAGAGCTAAGCTGGAAACAGCATAGCAAGT
TTAAATAAGGCTAGTCCGTTATCAACTCGAGAGAGTGGCACCGAGTCGGTGCTTG
TCTGAAGCAATCGCTTCCTCCTGAAAATAACTCTCTTTGACGCGGTTCTATCTAGT
TACGCGTTAAACCAACTAGAAATTTTTT
RUNX1 +5G-T cr289 evopreQ1
(SEQ ID NO: 281)
GCATTTTCAGGAGGAAGCGAGTTTAAGAGCTAAGCTGGAAACAGCATAGCAAGT
TTAAATAAGGCTAGTCCGTTATCAACTGGAAACAGTGGCACCGAGTCGGTGCTTG
TCTGAAGCAATCGCTTCCTCCTGAAAATAACTCTCTTTGACGCGGTTCTATCTAGT
TACGCGTTAAACCAACTAGAAATTTTTT
RUNX1 +5G-T cr622 evopreQ1
(SEQ ID NO: 282)
GCATTTTCAGGAGGAAGCGAGTTTAAGAGCTAAGCTGGAAACAGCATAGCAAGT
TTAAATAAGGCTAGTCCGTTATCAGCTGGAAACAGCGGCACCGAGTCGGTGCTT
GTCTGAAGCAATCGCTTCCTCCTGAAAATAACTCTCTTTGACGCGGTTCTATCTA
GTTACGCGTTAAACCAACTAGAAATTTTTT
RUNX1 +5G-T cr772 evopreQ1
(SEQ ID NO: 283)
GCATTTTCAGGAGGAAGCGAGTTTAAGAGCTAAGCTGGAAACAGCATAGCAAGT
TTAAATAAGGCTAGTCCGTTATCAACTCGAAAGAGTGGCACCGAGTCGGTGCTTG
TCTGAAGCAATCGCTTCCTCCTGAAAATAACTCTCTTTGACGCGGTTCTATCTAGT
TACGCGTTAAACCAACTAGAAATTTTTT
RUNX1 +5G-T cr532 evopreQ1
(SEQ ID NO: 284)
GCATTTTCAGGAGGAAGCGAGTTTAAGAGCTAAGCTGGAAACAGCATAGCAAGT
TTAAATAAGGCTAGTCCGTTATCAACGTGAAAACGTGACTCCGAGTCGGAGTTTG
TCTGAAGCAATCGCTTCCTCCTGAAAATAACTCTCTTTGACGCGGTTCTATCTAGT
TACGCGTTAAACCAACTAGAAATTTTTT
RUNX1 +5G-T cr961 evopreQ1
(SEQ ID NO: 285)
GCATTTTCAGGAGGAAGCGAGTTTAAGAGCTAAGCTGGAAACAGCATAGCAAGT
TTAAATAAGGCTAGTCCGTTATCAACTCGAAAGAGTGCAACCGAGTCGGTTGTTG
TCTGAAGCAATCGCTTCCTCCTGAAAATAACTCTCTTTGACGCGGTTCTATCTAGT
TACGCGTTAAACCAACTAGAAATTTTTT
RUNX1 +5G-T flip and extension scaffold evopreQ1
(SEQ ID NO: 286)
GCATTTTCAGGAGGAAGCGAGTTTAAGAGCTAAGCTGGAAACAGCATAGCAAGT
TTAAATAAGGCTAGTCCGTTATCAACTTGAAAAAGTGGCACCGAGTCGGTGCTTG
TCTGAAGCAATCGCTTCCTCCTGAAAATAACTCTCTTTGACGCGGTTCTATCTAGT
TACGCGTTAAACCAACTAGAAATTTTTT
DNMT1 1-15del standard scaffold evopreQ1
(SEQ ID NO: 287)
GATTCCTGGTGCCAGAAACAGTTTTAGAGCTAGAAATAGCAAGTTAAAATAAGG
CTAGTCCGTTATCAACTTGAAAAAGTGGCACCGAGTCGGTGCTGCTAAGGACTA
GTTCTGCCCTTCTGGCACCAGGACCTCTTCTTTGACGCGGTTCTATCTAGTTACGC
GTTAAACCAACTAGAAATTTTTT
DNMT1 1-15del cr748 evopreQ1
(SEQ ID NO: 288)
GATTCCTGGTGCCAGAAACAGTTTAAGAGCTAAGCTGGAAACAGCATAGCAAGT
TTAAATAAGGCTAGTCCGTTATCAACTCGAGAGAGTGGCACCGAGTCGGTGCTTG
CTAAGGACTAGTTCTGCCCTTCTGGCACCAGGACCTCTTCTTTGACGCGGTTCTAT
CTAGTTACGCGTTAAACCAACTAGAAATTTTTT
DNMT1 1-15del cr289 evopreQ1
(SEQ ID NO: 289)
GATTCCTGGTGCCAGAAACAGTTTAAGAGCTAAGCTGGAAACAGCATAGCAAGT
TTAAATAAGGCTAGTCCGTTATCAACTGGAAACAGTGGCACCGAGTCGGTGCTTG
CTAAGGACTAGTTCTGCCCTTCTGGCACCAGGACCTCTTCTTTGACGCGGTTCTAT
CTAGTTACGCGTTAAACCAACTAGAAATTTTTT
DNMT1 1-15del cr622 evopreQ1
(SEQ ID NO: 290)
GATTCCTGGTGCCAGAAACAGTTTAAGAGCTAAGCTGGAAACAGCATAGCAAGT
TTAAATAAGGCTAGTCCGTTATCAGCTGGAAACAGCGGCACCGAGTCGGTGCTT
GCTAAGGACTAGTTCTGCCCTTCTGGCACCAGGACCTCTTCTTTGACGCGGTTCT
ATCTAGTTACGCGTTAAACCAACTAGAAATTTTTT
DNMT1 1-15del cr772 evopreQ1
(SEQ ID NO: 291)
GATTCCTGGTGCCAGAAACAGTTTAAGAGCTAAGCTGGAAACAGCATAGCAAGT
TTAAATAAGGCTAGTCCGTTATCAACTCGAAAGAGTGGCACCGAGTCGGTGCTTG
CTAAGGACTAGTTCTGCCCTTCTGGCACCAGGACCTCTTCTTTGACGCGGTTCTAT
CTAGTTACGCGTTAAACCAACTAGAAATTTTTT
DNMT1 1-15del cr532 evopreQ1
(SEQ ID NO: 292)
GATTCCTGGTGCCAGAAACAGTTTAAGAGCTAAGCTGGAAACAGCATAGCAAGT
TTAAATAAGGCTAGTCCGTTATCAACGTGAAAACGTGACTCCGAGTCGGAGTTTG
CTAAGGACTAGTTCTGCCCTTCTGGCACCAGGACCTCTTCTTTGACGCGGTTCTAT
CTAGTTACGCGTTAAACCAACTAGAAATTTTTT
DNMT1 1-15del cr961 evopreQ1
(SEQ ID NO: 293)
GATTCCTGGTGCCAGAAACAGTTTAAGAGCTAAGCTGGAAACAGCATAGCAAGT
TTAAATAAGGCTAGTCCGTTATCAACTCGAAAGAGTGCAACCGAGTCGGTTGTTG
CTAAGGACTAGTTCTGCCCTTCTGGCACCAGGACCTCTTCTTTGACGCGGTTCTAT
CTAGTTACGCGTTAAACCAACTAGAAATTTTTT
DNMT1 1-15del flip and extension scaffold evopreQ1
(SEQ ID NO: 294)
GATTCCTGGTGCCAGAAACAGTTTAAGAGCTAAGCTGGAAACAGCATAGCAAGT
TTAAATAAGGCTAGTCCGTTATCAACTTGAAAAAGTGGCACCGAGTCGGTGCTTG
CTAAGGACTAGTTCTGCCCTTCTGGCACCAGGACCTCTTCTTTGACGCGGTTCTAT
CTAGTTACGCGTTAAACCAACTAGAAATTTTTT
DNMT1 +5 G--T standard scaffold evopreQ1
(SEQ ID NO: 295)
GATTCCTGGTGCCAGAAACAGTTTTAGAGCTAGAAATAGCAAGTTAAAATAAGG
CTAGTCCGTTATCAACTTGAAAAAGTGGCACCGAGTCGGTGCGTCACCACTGTTT
CTGGCACCAGGACCTCTTCTTTGACGCGGTTCTATCTAGTTACGCGTTAAACCAA
CTAGAAATTTTTT
DNMT1 +5 G--T cr748 evopreQ1
(SEQ ID NO: 296)
GATTCCTGGTGCCAGAAACAGTTTAAGAGCTAAGCTGGAAACAGCATAGCAAGT
TTAAATAAGGCTAGTCCGTTATCAACTCGAGAGAGTGGCACCGAGTCGGTGCTGT
CACCACTGTTTCTGGCACCAGGACCTCTTCTTTGACGCGGTTCTATCTAGTTACGC
GTTAAACCAACTAGAAATTTTTT
DNMT1 +5 G--T cr289 evopreQ1
(SEQ ID NO: 297)
GATTCCTGGTGCCAGAAACAGTTTAAGAGCTAAGCTGGAAACAGCATAGCAAGT
TTAAATAAGGCTAGTCCGTTATCAACTGGAAACAGTGGCACCGAGTCGGTGCTGT
CACCACTGTTTCTGGCACCAGGACCTCTTCTTTGACGCGGTTCTATCTAGTTACGC
GTTAAACCAACTAGAAATTTTTT
DNMT1 +5 G--T cr622 evopreQ1
(SEQ ID NO: 298)
GATTCCTGGTGCCAGAAACAGTTTAAGAGCTAAGCTGGAAACAGCATAGCAAGT
TTAAATAAGGCTAGTCCGTTATCAGCTGGAAACAGCGGCACCGAGTCGGTGCTG
TCACCACTGTTTCTGGCACCAGGACCTCTTCTTTGACGCGGTTCTATCTAGTTACG
CGTTAAACCAACTAGAAATTTTTT
DNMT1 +5 G--T cr772 evopreQ1
(SEQ ID NO: 299)
GATTCCTGGTGCCAGAAACAGTTTAAGAGCTAAGCTGGAAACAGCATAGCAAGT
TTAAATAAGGCTAGTCCGTTATCAACTCGAAAGAGTGGCACCGAGTCGGTGCTGT
CACCACTGTTTCTGGCACCAGGACCTCTTCTTTGACGCGGTTCTATCTAGTTACGC
GTTAAACCAACTAGAAATTTTTT
DNMT1 +5 G--T cr532 evopreQ1
(SEQ ID NO: 300)
GATTCCTGGTGCCAGAAACAGTTTAAGAGCTAAGCTGGAAACAGCATAGCAAGT
TTAAATAAGGCTAGTCCGTTATCAACGTGAAAACGTGACTCCGAGTCGGAGTTGT
CACCACTGTTTCTGGCACCAGGACCTCTTCTTTGACGCGGTTCTATCTAGTTACGC
GTTAAACCAACTAGAAATTTTTT
DNMT1 +5 G--T cr961 evopreQ1
(SEQ ID NO: 301)
GATTCCTGGTGCCAGAAACAGTTTAAGAGCTAAGCTGGAAACAGCATAGCAAGT
TTAAATAAGGCTAGTCCGTTATCAACTCGAAAGAGTGCAACCGAGTCGGTTGTGT
CACCACTGTTTCTGGCACCAGGACCTCTTCTTTGACGCGGTTCTATCTAGTTACGC
GTTAAACCAACTAGAAATTTTTT
DNMT1 +5 G--T flip and extension scaffold evopreQ1
(SEQ ID NO: 302)
GATTCCTGGTGCCAGAAACAGTTTAAGAGCTAAGCTGGAAACAGCATAGCAAGT
TTAAATAAGGCTAGTCCGTTATCAACTTGAAAAAGTGGCACCGAGTCGGTGCTGT
CACCACTGTTTCTGGCACCAGGACCTCTTCTTTGACGCGGTTCTATCTAGTTACGC
GTTAAACCAACTAGAAATTTTTT
FANCF 1-15del standard scaffold evopreQ1
(SEQ ID NO: 303)
GGAATCCCTTCTGCAGCACCGTTTTAGAGCTAGAAATAGCAAGTTAAAATAAGG
CTAGTCCGTTATCAACTTGAAAAAGTGGCACCGAGTCGGTGCTAGTGCTTGAGAC
CGCCAGAAGCTCGGGCTGCAGAAGGGACAATCACTTTGACGCGGTTCTATCTAG
TTACGCGTTAAACCAACTAGAAATTTTTT
FANCF 1-15del cr748 evopreQ1
(SEQ ID NO: 304)
GGAATCCCTTCTGCAGCACCGTTTAAGAGCTAAGCTGGAAACAGCATAGCAAGT
TTAAATAAGGCTAGTCCGTTATCAACTCGAGAGAGTGGCACCGAGTCGGTGCTTA
GTGCTTGAGACCGCCAGAAGCTCGGGCTGCAGAAGGGACAATCACTTTGACGCG
GTTCTATCTAGTTACGCGTTAAACCAACTAGAAATTTTTT
FANCF 1-15del cr289 evopreQ1
(SEQ ID NO: 305)
GGAATCCCTTCTGCAGCACCGTTTAAGAGCTAAGCTGGAAACAGCATAGCAAGT
TTAAATAAGGCTAGTCCGTTATCAACTGGAAACAGTGGCACCGAGTCGGTGCTTA
GTGCTTGAGACCGCCAGAAGCTCGGGCTGCAGAAGGGACAATCACTTTGACGCG
GTTCTATCTAGTTACGCGTTAAACCAACTAGAAATTTTTT
FANCF 1-15del cr622 evopreQ1
(SEQ ID NO: 306)
GGAATCCCTTCTGCAGCACCGTTTAAGAGCTAAGCTGGAAACAGCATAGCAAGT
TTAAATAAGGCTAGTCCGTTATCAGCTGGAAACAGCGGCACCGAGTCGGTGCTT
AGTGCTTGAGACCGCCAGAAGCTCGGGCTGCAGAAGGGACAATCACTTTGACGC
GGTTCTATCTAGTTACGCGTTAAACCAACTAGAAATTTTTT
FANCF 1-15del cr772 evopreQ1
(SEQ ID NO: 307)
GGAATCCCTTCTGCAGCACCGTTTAAGAGCTAAGCTGGAAACAGCATAGCAAGT
TTAAATAAGGCTAGTCCGTTATCAACTCGAAAGAGTGGCACCGAGTCGGTGCTTA
GTGCTTGAGACCGCCAGAAGCTCGGGCTGCAGAAGGGACAATCACTTTGACGCG
GTTCTATCTAGTTACGCGTTAAACCAACTAGAAATTTTTT
FANCF 1-15del cr532 evopreQ1
(SEQ ID NO: 308)
GGAATCCCTTCTGCAGCACCGTTTAAGAGCTAAGCTGGAAACAGCATAGCAAGT
TTAAATAAGGCTAGTCCGTTATCAACGTGAAAACGTGACTCCGAGTCGGAGTTTA
GTGCTTGAGACCGCCAGAAGCTCGGGCTGCAGAAGGGACAATCACTTTGACGCG
GTTCTATCTAGTTACGCGTTAAACCAACTAGAAATTTTTT
FANCF 1-15del cr961 evopreQ1
(SEQ ID NO: 309)
GGAATCCCTTCTGCAGCACCGTTTAAGAGCTAAGCTGGAAACAGCATAGCAAGT
TTAAATAAGGCTAGTCCGTTATCAACTCGAAAGAGTGCAACCGAGTCGGTTGTTA
GTGCTTGAGACCGCCAGAAGCTCGGGCTGCAGAAGGGACAATCACTTTGACGCG
GTTCTATCTAGTTACGCGTTAAACCAACTAGAAATTTTTT
FANCF 1-15del flip and extension scaffold evopreQ1
(SEQ ID NO: 310)
GGAATCCCTTCTGCAGCACCGTTTAAGAGCTAAGCTGGAAACAGCATAGCAAGT
TTAAATAAGGCTAGTCCGTTATCAACTTGAAAAAGTGGCACCGAGTCGGTGCTTA
GTGCTTGAGACCGCCAGAAGCTCGGGCTGCAGAAGGGACAATCACTTTGACGCG
GTTCTATCTAGTTACGCGTTAAACCAACTAGAAATTTTTT
FANCF +5 G--T cr748 evopreQ1
(SEQ ID NO: 311)
GGAATCCCTTCTGCAGCACCGTTTAAGAGCTAAGCTGGAAACAGCATAGCAAGT
TTAAATAAGGCTAGTCCGTTATCAACTCGAGAGAGTGGCACCGAGTCGGTGCTG
GAAAAGCGATCAAGGTGCTGCAGAAGGGACAATCACTTTGACGCGGTTCTATCT
AGTTACGCGTTAAACCAACTAGAAATTTTTT
FANCF +5 G--T cr289 evopreQ1
(SEQ ID NO: 312)
GGAATCCCTTCTGCAGCACCGTTTAAGAGCTAAGCTGGAAACAGCATAGCAAGT
TTAAATAAGGCTAGTCCGTTATCAACTGGAAACAGTGGCACCGAGTCGGTGCTG
GAAAAGCGATCAAGGTGCTGCAGAAGGGACAATCACTTTGACGCGGTTCTATCT
AGTTACGCGTTAAACCAACTAGAAATTTTTT
FANCF +5 G--T cr622 evopreQ1
(SEQ ID NO: 313)
GGAATCCCTTCTGCAGCACCGTTTAAGAGCTAAGCTGGAAACAGCATAGCAAGT
TTAAATAAGGCTAGTCCGTTATCAGCTGGAAACAGCGGCACCGAGTCGGTGCTG
GAAAAGCGATCAAGGTGCTGCAGAAGGGACAATCACTTTGACGCGGTTCTATCT
AGTTACGCGTTAAACCAACTAGAAATTTTTT
FANCF +5 G--T cr772 evopreQ1
(SEQ ID NO: 314)
GGAATCCCTTCTGCAGCACCGTTTAAGAGCTAAGCTGGAAACAGCATAGCAAGT
TTAAATAAGGCTAGTCCGTTATCAACTCGAAAGAGTGGCACCGAGTCGGTGCTG
GAAAAGCGATCAAGGTGCTGCAGAAGGGACAATCACTTTGACGCGGTTCTATCT
AGTTACGCGTTAAACCAACTAGAAATTTTTT
FANCF +5 G--T cr532 evopreQ1
(SEQ ID NO: 315)
GGAATCCCTTCTGCAGCACCGTTTAAGAGCTAAGCTGGAAACAGCATAGCAAGT
TTAAATAAGGCTAGTCCGTTATCAACGTGAAAACGTGACTCCGAGTCGGAGTTG
GAAAAGCGATCAAGGTGCTGCAGAAGGGACAATCACTTTGACGCGGTTCTATCT
AGTTACGCGTTAAACCAACTAGAAATTTTTT
FANCF +5 G--T cr961 evopreQ1
(SEQ ID NO: 316)
GGAATCCCTTCTGCAGCACCGTTTAAGAGCTAAGCTGGAAACAGCATAGCAAGT
TTAAATAAGGCTAGTCCGTTATCAACTCGAAAGAGTGCAACCGAGTCGGTTGTG
GAAAAGCGATCAAGGTGCTGCAGAAGGGACAATCACTTTGACGCGGTTCTATCT
AGTTACGCGTTAAACCAACTAGAAATTTTTT
FANCF +5 G--T flip and extension scaffold evopreQ1
(SEQ ID NO: 317)
GGAATCCCTTCTGCAGCACCGTTTAAGAGCTAAGCTGGAAACAGCATAGCAAGT
TTAAATAAGGCTAGTCCGTTATCAACTTGAAAAAGTGGCACCGAGTCGGTGCTG
GAAAAGCGATCAAGGTGCTGCAGAAGGGACAATCACTTTGACGCGGTTCTATCT
AGTTACGCGTTAAACCAACTAGAAATTTTTT
EMX1 1-15del standard scaffold evopreQ1
(SEQ ID NO: 318)
GAGTCCGAGCAGAAGAAGAAGTTTTAGAGCTAGAAATAGCAAGTTAAAATAAGG
CTAGTCCGTTATCAACTTGAAAAAGTGGCACCGAGTCGGTGCTCGTGGCAATGCG
CCACCGGTTGATGTTCTTCTGCTCGGAAACAATCTTTGACGCGGTTCTATCTAGTT
ACGCGTTAAACCAACTAGAAATTTTTT
EMX1 1-15del cr748 evopreQ1
(SEQ ID NO: 319)
GAGTCCGAGCAGAAGAAGAAGTTTAAGAGCTAAGCTGGAAACAGCATAGCAAG
TTTAAATAAGGCTAGTCCGTTATCAACTCGAGAGAGTGGCACCGAGTCGGTGCTT
CGTGGCAATGCGCCACCGGTTGATGTTCTTCTGCTCGGAAACAATCTTTGACGCG
GTTCTATCTAGTTACGCGTTAAACCAACTAGAAATTTTTT
EMX1 1-15del cr289 evopreQ1
(SEQ ID NO: 320)
GAGTCCGAGCAGAAGAAGAAGTTTAAGAGCTAAGCTGGAAACAGCATAGCAAG
TTTAAATAAGGCTAGTCCGTTATCAACTGGAAACAGTGGCACCGAGTCGGTGCTT
CGTGGCAATGCGCCACCGGTTGATGTTCTTCTGCTCGGAAACAATCTTTGACGCG
GTTCTATCTAGTTACGCGTTAAACCAACTAGAAATTTTTT
EMX1 1-15del cr622 evopreQ1
(SEQ ID NO: 321)
GAGTCCGAGCAGAAGAAGAAGTTTAAGAGCTAAGCTGGAAACAGCATAGCAAG
TTTAAATAAGGCTAGTCCGTTATCAGCTGGAAACAGCGGCACCGAGTCGGTGCTT
CGTGGCAATGCGCCACCGGTTGATGTTCTTCTGCTCGGAAACAATCTTTGACGCG
GTTCTATCTAGTTACGCGTTAAACCAACTAGAAATTTTTT
EMX1 1-15del cr772 evopreQ1
(SEQ ID NO: 322)
GAGTCCGAGCAGAAGAAGAAGTTTAAGAGCTAAGCTGGAAACAGCATAGCAAG
TTTAAATAAGGCTAGTCCGTTATCAACTCGAAAGAGTGGCACCGAGTCGGTGCTT
CGTGGCAATGCGCCACCGGTTGATGTTCTTCTGCTCGGAAACAATCTTTGACGCG
GTTCTATCTAGTTACGCGTTAAACCAACTAGAAATTTTTT
EMX1 1-15del cr532 evopreQ1
(SEQ ID NO: 323)
GAGTCCGAGCAGAAGAAGAAGTTTAAGAGCTAAGCTGGAAACAGCATAGCAAG
TTTAAATAAGGCTAGTCCGTTATCAACGTGAAAACGTGACTCCGAGTCGGAGTTT
CGTGGCAATGCGCCACCGGTTGATGTTCTTCTGCTCGGAAACAATCTTTGACGCG
GTTCTATCTAGTTACGCGTTAAACCAACTAGAAATTTTTT
EMX1 1-15del cr961 evopreQ1
(SEQ ID NO: 324)
GAGTCCGAGCAGAAGAAGAAGTTTAAGAGCTAAGCTGGAAACAGCATAGCAAG
TTTAAATAAGGCTAGTCCGTTATCAACTCGAAAGAGTGCAACCGAGTCGGTTGTT
CGTGGCAATGCGCCACCGGTTGATGTTCTTCTGCTCGGAAACAATCTTTGACGCG
GTTCTATCTAGTTACGCGTTAAACCAACTAGAAATTTTTT
EMX1 1-15del flip and extension scaffold evopreQ1
(SEQ ID NO: 325)
GAGTCCGAGCAGAAGAAGAAGTTTAAGAGCTAAGCTGGAAACAGCATAGCAAG
TTTAAATAAGGCTAGTCCGTTATCAACTTGAAAAAGTGGCACCGAGTCGGTGCTT
CGTGGCAATGCGCCACCGGTTGATGTTCTTCTGCTCGGAAACAATCTTTGACGCG
GTTCTATCTAGTTACGCGTTAAACCAACTAGAAATTTTTT
EMX1 +5 G--T standard scaffold evopreQ1
(SEQ ID NO: 326)
GAGTCCGAGCAGAAGAAGAAGTTTTAGAGCTAGAAATAGCAAGTTAAAATAAGG
CTAGTCCGTTATCAACTTGAAAAAGTGGCACCGAGTCGGTGCGTGATGGGAGCA
CTTCTTCTTCTGCTCGGAAACAATCTTTGACGCGGTTCTATCTAGTTACGCGTTAA
ACCAACTAGAAATTTTTT
EMX1 +5 G--T cr748 evopreQ1
(SEQ ID NO: 327)
GAGTCCGAGCAGAAGAAGAAGTTTAAGAGCTAAGCTGGAAACAGCATAGCAAG
TTTAAATAAGGCTAGTCCGTTATCAACTCGAGAGAGTGGCACCGAGTCGGTGCTG
TGATGGGAGCACTTCTTCTTCTGCTCGGAAACAATCTTTGACGCGGTTCTATCTA
GTTACGCGTTAAACCAACTAGAAATTTTTT
EMX1 +5 G--T cr289 evopreQ1
(SEQ ID NO: 328)
GAGTCCGAGCAGAAGAAGAAGTTTAAGAGCTAAGCTGGAAACAGCATAGCAAG
TTTAAATAAGGCTAGTCCGTTATCAACTGGAAACAGTGGCACCGAGTCGGTGCTG
TGATGGGAGCACTTCTTCTTCTGCTCGGAAACAATCTTTGACGCGGTTCTATCTA
GTTACGCGTTAAACCAACTAGAAATTTTTT
EMX1 +5 G--T cr622 evopreQ1
(SEQ ID NO: 329)
GAGTCCGAGCAGAAGAAGAAGTTTAAGAGCTAAGCTGGAAACAGCATAGCAAG
TTTAAATAAGGCTAGTCCGTTATCAGCTGGAAACAGCGGCACCGAGTCGGTGCT
GTGATGGGAGCACTTCTTCTTCTGCTCGGAAACAATCTTTGACGCGGTTCTATCT
AGTTACGCGTTAAACCAACTAGAAATTTTTT
EMX1 +5 G--T cr772 evopreQ1
(SEQ ID NO: 330)
GAGTCCGAGCAGAAGAAGAAGTTTAAGAGCTAAGCTGGAAACAGCATAGCAAG
TTTAAATAAGGCTAGTCCGTTATCAACTCGAAAGAGTGGCACCGAGTCGGTGCTG
TGATGGGAGCACTTCTTCTTCTGCTCGGAAACAATCTTTGACGCGGTTCTATCTA
GTTACGCGTTAAACCAACTAGAAATTTTTT
EMX1 +5 G--T cr532 evopreQ1
(SEQ ID NO: 331)
GAGTCCGAGCAGAAGAAGAAGTTTAAGAGCTAAGCTGGAAACAGCATAGCAAG
TTTAAATAAGGCTAGTCCGTTATCAACGTGAAAACGTGACTCCGAGTCGGAGTTG
TGATGGGAGCACTTCTTCTTCTGCTCGGAAACAATCTTTGACGCGGTTCTATCTA
GTTACGCGTTAAACCAACTAGAAATTTTTT
EMX1 +5 G--T cr961 evopreQ1
(SEQ ID NO: 332)
GAGTCCGAGCAGAAGAAGAAGTTTAAGAGCTAAGCTGGAAACAGCATAGCAAG
TTTAAATAAGGCTAGTCCGTTATCAACTCGAAAGAGTGCAACCGAGTCGGTTGTG
TGATGGGAGCACTTCTTCTTCTGCTCGGAAACAATCTTTGACGCGGTTCTATCTA
GTTACGCGTTAAACCAACTAGAAATTTTTT
EMX1 +5 G--T flip and extension scaffold evopreQ1
(SEQ ID NO: 333)
GAGTCCGAGCAGAAGAAGAAGTTTAAGAGCTAAGCTGGAAACAGCATAGCAAG
TTTAAATAAGGCTAGTCCGTTATCAACTTGAAAAAGTGGCACCGAGTCGGTGCTG
TGATGGGAGCACTTCTTCTTCTGCTCGGAAACAATCTTTGACGCGGTTCTATCTA
GTTACGCGTTAAACCAACTAGAAATTTTTT
RNF2 +1FLAG standard scaffold evopreQ1
(SEQ ID NO: 334)
GTCATCTTAGTCATTACCTGGTTTTAGAGCTAGAAATAGCAAGTTAAAATAAGGC
TAGTCCGTTATCAACTTGAAAAAGTGGCACCGAGTCGGTGCTGAGTTACAACGA
ACACCTCAGCTTATCGTCGTCATCCTTGTAATCGTAATGACTAAGATGTCATCTCT
TTGACGCGGTTCTATCTAGTTACGCGTTAAACCAACTAGAAATTTTTT
RNF2 + 1FLAG cr748 evopreQ1
(SEQ ID NO: 335)
GTCATCTTAGTCATTACCTGGTTTAAGAGCTAAGCTGGAAACAGCATAGCAAGTT
TAAATAAGGCTAGTCCGTTATCAACTCGAGAGAGTGGCACCGAGTCGGTGCTTG
AGTTACAACGAACACCTCAGCTTATCGTCGTCATCCTTGTAATCGTAATGACTAA
GATGTCATCTCTTTGACGCGGTTCTATCTAGTTACGCGTTAAACCAACTAGAAAT
TTTTT
RNF2 + 1FLAG cr289 evopreQ1
(SEQ ID NO: 336)
GTCATCTTAGTCATTACCTGGTTTAAGAGCTAAGCTGGAAACAGCATAGCAAGTT
TAAATAAGGCTAGTCCGTTATCAACTGGAAACAGTGGCACCGAGTCGGTGCTTG
AGTTACAACGAACACCTCAGCTTATCGTCGTCATCCTTGTAATCGTAATGACTAA
GATGTCATCTCTTTGACGCGGTTCTATCTAGTTACGCGTTAAACCAACTAGAAAT
TTTTT
(SEQ ID NO: 337)
RNF2 + 1FLAG cr622 evopreQ1
GTCATCTTAGTCATTACCTGGTTTAAGAGCTAAGCTGGAAACAGCATAGCAAGTT
TAAATAAGGCTAGTCCGTTATCAGCTGGAAACAGCGGCACCGAGTCGGTGCTTG
AGTTACAACGAACACCTCAGCTTATCGTCGTCATCCTTGTAATCGTAATGACTAA
GATGTCATCTCTTTGACGCGGTTCTATCTAGTTACGCGTTAAACCAACTAGAAAT
TTTTT
RNF2 + 1FLAG cr772 evopreQ1
(SEQ ID NO: 338)
GTCATCTTAGTCATTACCTGGTTTAAGAGCTAAGCTGGAAACAGCATAGCAAGTT
TAAATAAGGCTAGTCCGTTATCAACTCGAAAGAGTGGCACCGAGTCGGTGCTTG
AGTTACAACGAACACCTCAGCTTATCGTCGTCATCCTTGTAATCGTAATGACTAA
GATGTCATCTCTTTGACGCGGTTCTATCTAGTTACGCGTTAAACCAACTAGAAAT
TTTTT
RNF2 + 1FLAG cr532 evopreQ1
(SEQ ID NO: 339)
GTCATCTTAGTCATTACCTGGTTTAAGAGCTAAGCTGGAAACAGCATAGCAAGTT
TAAATAAGGCTAGTCCGTTATCAACGTGAAAACGTGACTCCGAGTCGGAGTTTG
AGTTACAACGAACACCTCAGCTTATCGTCGTCATCCTTGTAATCGTAATGACTAA
GATGTCATCTCTTTGACGCGGTTCTATCTAGTTACGCGTTAAACCAACTAGAAAT
TTTTT
RNF2 + 1FLAG cr961 evopreQ1
(SEQ ID NO: 340)
GTCATCTTAGTCATTACCTGGTTTAAGAGCTAAGCTGGAAACAGCATAGCAAGTT
TAAATAAGGCTAGTCCGTTATCAACTCGAAAGAGTGCAACCGAGTCGGTTGTTG
AGTTACAACGAACACCTCAGCTTATCGTCGTCATCCTTGTAATCGTAATGACTAA
GATGTCATCTCTTTGACGCGGTTCTATCTAGTTACGCGTTAAACCAACTAGAAAT
TTTTT
RNF2 + 1FLAG flip and extension scaffold evopreQ1
(SEQ ID NO: 341)
GTCATCTTAGTCATTACCTGGTTTAAGAGCTAAGCTGGAAACAGCATAGCAAGTT
TAAATAAGGCTAGTCCGTTATCAACTTGAAAAAGTGGCACCGAGTCGGTGCTTG
AGTTACAACGAACACCTCAGCTTATCGTCGTCATCCTTGTAATCGTAATGACTAA
GATGTCATCTCTTTGACGCGGTTCTATCTAGTTACGCGTTAAACCAACTAGAAAT
TTTTT
VEGFA +5 G--T cr748 evopreQ1
(SEQ ID NO: 342)
GATGTCTGCAGGCCAGATGAGTTTAAGAGCTAAGCTGGAAACAGCATAGCAAGT
TTAAATAAGGCTAGTCCGTTATCAACTCGAGAGAGTGGCACCGAGTCGGTGCTA
ATGTGCCATCTGGAGCACTCATCTGGCCTGCAGAACAATCTCTTGACGCGGTTCT
ATCTAGTTACGCGTTAAACCAACTAGAAATTTTTT
VEGFA +5 G--T cr289 evopreQ1
(SEQ ID NO: 343)
GATGTCTGCAGGCCAGATGAGTTTAAGAGCTAAGCTGGAAACAGCATAGCAAGT
TTAAATAAGGCTAGTCCGTTATCAACTGGAAACAGTGGCACCGAGTCGGTGCTA
ATGTGCCATCTGGAGCACTCATCTGGCCTGCAGAACAATCTCTTGACGCGGTTCT
ATCTAGTTACGCGTTAAACCAACTAGAAATTTTTT
VEGFA +5 G--T cr622 evopreQ1
(SEQ ID NO: 344)
GATGTCTGCAGGCCAGATGAGTTTAAGAGCTAAGCTGGAAACAGCATAGCAAGT
TTAAATAAGGCTAGTCCGTTATCAGCTGGAAACAGCGGCACCGAGTCGGTGCTA
ATGTGCCATCTGGAGCACTCATCTGGCCTGCAGAACAATCTCTTGACGCGGTTCT
ATCTAGTTACGCGTTAAACCAACTAGAAATTTTTT
VEGFA +5 G--T cr772 evopreQ1
(SEQ ID NO: 345)
GATGTCTGCAGGCCAGATGAGTTTAAGAGCTAAGCTGGAAACAGCATAGCAAGT
TTAAATAAGGCTAGTCCGTTATCAACTCGAAAGAGTGGCACCGAGTCGGTGCTA
ATGTGCCATCTGGAGCACTCATCTGGCCTGCAGAACAATCTCTTGACGCGGTTCT
ATCTAGTTACGCGTTAAACCAACTAGAAATTTTTT
VEGFA +5 G--T cr532 evopreQ1
(SEQ ID NO: 346)
GATGTCTGCAGGCCAGATGAGTTTAAGAGCTAAGCTGGAAACAGCATAGCAAGT
TTAAATAAGGCTAGTCCGTTATCAACGTGAAAACGTGACTCCGAGTCGGAGTTA
ATGTGCCATCTGGAGCACTCATCTGGCCTGCAGAACAATCTCTTGACGCGGTTCT
ATCTAGTTACGCGTTAAACCAACTAGAAATTTTTT
VEGFA +5 G--T cr961 evopreQ1
(SEQ ID NO: 347)
GATGTCTGCAGGCCAGATGAGTTTAAGAGCTAAGCTGGAAACAGCATAGCAAGT
TTAAATAAGGCTAGTCCGTTATCAACTCGAAAGAGTGCAACCGAGTCGGTTGTA
ATGTGCCATCTGGAGCACTCATCTGGCCTGCAGAACAATCTCTTGACGCGGTTCT
ATCTAGTTACGCGTTAAACCAACTAGAAATTTTTT
VEGFA +5 G--T flip and extension scaffold evopreQ1
(SEQ ID NO: 348)
GATGTCTGCAGGCCAGATGAGTTTAAGAGCTAAGCTGGAAACAGCATAGCAAGT
TTAAATAAGGCTAGTCCGTTATCAACTTGAAAAAGTGGCACCGAGTCGGTGCTA
ATGTGCCATCTGGAGCACTCATCTGGCCTGCAGAACAATCTCTTGACGCGGTTCT
ATCTAGTTACGCGTTAAACCAACTAGAAATTTTTT
VEGFA + 1FLAG standard scaffold evopreQ1
(SEQ ID NO: 349)
GATGTCTGCAGGCCAGATGAGTTTTAGAGCTAGAAATAGCAAGTTAAAATAAGG
CTAGTCCGTTATCAACTTGAAAAAGTGGCACCGAGTCGGTGCAATGTGCCATCTG
GAGCACTCACTTATCGTCGTCATCCTTGTAATCTCTGGCCTGCAGAACAATCTCTT
GACGCGGTTCTATCTAGTTACGCGTTAAACCAACTAGAAATTTTTT
VEGFA + 1FLAG cr748 evopreQ1
(SEQ ID NO: 350)
GATGTCTGCAGGCCAGATGAGTTTAAGAGCTAAGCTGGAAACAGCATAGCAAGT
TTAAATAAGGCTAGTCCGTTATCAACTCGAGAGAGTGGCACCGAGTCGGTGCTA
ATGTGCCATCTGGAGCACTCACTTATCGTCGTCATCCTTGTAATCTCTGGCCTGCA
GAACAATCTCTTGACGCGGTTCTATCTAGTTACGCGTTAAACCAACTAGAAATTT
TTT
VEGFA + 1FLAG cr289 evopreQ1
(SEQ ID NO: 351)
GATGTCTGCAGGCCAGATGAGTTTAAGAGCTAAGCTGGAAACAGCATAGCAAGT
TTAAATAAGGCTAGTCCGTTATCAACTGGAAACAGTGGCACCGAGTCGGTGCTA
ATGTGCCATCTGGAGCACTCACTTATCGTCGTCATCCTTGTAATCTCTGGCCTGCA
GAACAATCTCTTGACGCGGTTCTATCTAGTTACGCGTTAAACCAACTAGAAATTT
TTT
VEGFA + 1FLAG cr622 evopreQ1
(SEQ ID NO: 352)
GATGTCTGCAGGCCAGATGAGTTTAAGAGCTAAGCTGGAAACAGCATAGCAAGT
TTAAATAAGGCTAGTCCGTTATCAGCTGGAAACAGCGGCACCGAGTCGGTGCTA
ATGTGCCATCTGGAGCACTCACTTATCGTCGTCATCCTTGTAATCTCTGGCCTGCA
GAACAATCTCTTGACGCGGTTCTATCTAGTTACGCGTTAAACCAACTAGAAATTT
TTT
VEGFA + 1FLAG cr772 evopreQ1
(SEQ ID NO: 353)
GATGTCTGCAGGCCAGATGAGTTTAAGAGCTAAGCTGGAAACAGCATAGCAAGT
TTAAATAAGGCTAGTCCGTTATCAACTCGAAAGAGTGGCACCGAGTCGGTGCTA
ATGTGCCATCTGGAGCACTCACTTATCGTCGTCATCCTTGTAATCTCTGGCCTGCA
GAACAATCTCTTGACGCGGTTCTATCTAGTTACGCGTTAAACCAACTAGAAATTT
TTT
VEGFA + 1FLAG cr532 evopreQ1
(SEQ ID NO: 354)
GATGTCTGCAGGCCAGATGAGTTTAAGAGCTAAGCTGGAAACAGCATAGCAAGT
TTAAATAAGGCTAGTCCGTTATCAACGTGAAAACGTGACTCCGAGTCGGAGTTA
ATGTGCCATCTGGAGCACTCACTTATCGTCGTCATCCTTGTAATCTCTGGCCTGCA
GAACAATCTCTTGACGCGGTTCTATCTAGTTACGCGTTAAACCAACTAGAAATTT
TTT
VEGFA + 1FLAG cr961 evopreQ1
(SEQ ID NO: 355)
GATGTCTGCAGGCCAGATGAGTTTAAGAGCTAAGCTGGAAACAGCATAGCAAGT
TTAAATAAGGCTAGTCCGTTATCAACTCGAAAGAGTGCAACCGAGTCGGTTGTA
ATGTGCCATCTGGAGCACTCACTTATCGTCGTCATCCTTGTAATCTCTGGCCTGCA
GAACAATCTCTTGACGCGGTTCTATCTAGTTACGCGTTAAACCAACTAGAAATTT
TTT
VEGFA + 1FLAG flip and extension scaffold evopreQ1
(SEQ ID NO: 356)
GATGTCTGCAGGCCAGATGAGTTTAAGAGCTAAGCTGGAAACAGCATAGCAAGT
TTAAATAAGGCTAGTCCGTTATCAACTTGAAAAAGTGGCACCGAGTCGGTGCTA
ATGTGCCATCTGGAGCACTCACTTATCGTCGTCATCCTTGTAATCTCTGGCCTGCA
GAACAATCTCTTGACGCGGTTCTATCTAGTTACGCGTTAAACCAACTAGAAATTT
TTT
VEGFA 1-15 del standard scaffold evopreQ1
(SEQ ID NO: 357)
GATGTCTGCAGGCCAGATGAGTTTTAGAGCTAGAAATAGCAAGTTAAAATAAGG
CTAGTCCGTTATCAACTTGAAAAAGTGGCACCGAGTCGGTGCGTGTGTCCCTCTG
ACAATGTGCTCTGGCCTGCAGAACAATCTCTTGACGCGGTTCTATCTAGTTACGC
GTTAAACCAACTAGAAATTTTTT
VEGFA 1-15 del cr748 evopreQ1
(SEQ ID NO: 358)
GATGTCTGCAGGCCAGATGAGTTTAAGAGCTAAGCTGGAAACAGCATAGCAAGT
TTAAATAAGGCTAGTCCGTTATCAACTCGAGAGAGTGGCACCGAGTCGGTGCTGT
GTGTCCCTCTGACAATGTGCTCTGGCCTGCAGAACAATCTCTTGACGCGGTTCTA
TCTAGTTACGCGTTAAACCAACTAGAAATTTTTT
VEGFA 1-15 del cr289 evopreQ1
(SEQ ID NO: 359)
GATGTCTGCAGGCCAGATGAGTTTAAGAGCTAAGCTGGAAACAGCATAGCAAGT
TTAAATAAGGCTAGTCCGTTATCAACTGGAAACAGTGGCACCGAGTCGGTGCTGT
GTGTCCCTCTGACAATGTGCTCTGGCCTGCAGAACAATCTCTTGACGCGGTTCTA
TCTAGTTACGCGTTAAACCAACTAGAAATTTTTT
VEGFA 1-15 del cr622 evopreQ1
(SEQ ID NO: 360)
GATGTCTGCAGGCCAGATGAGTTTAAGAGCTAAGCTGGAAACAGCATAGCAAGT
TTAAATAAGGCTAGTCCGTTATCAGCTGGAAACAGCGGCACCGAGTCGGTGCTG
TGTGTCCCTCTGACAATGTGCTCTGGCCTGCAGAACAATCTCTTGACGCGGTTCT
ATCTAGTTACGCGTTAAACCAACTAGAAATTTTTT
VEGFA 1-15 del cr772 evopreQ1
(SEQ ID NO: 361)
GATGTCTGCAGGCCAGATGAGTTTAAGAGCTAAGCTGGAAACAGCATAGCAAGT
TTAAATAAGGCTAGTCCGTTATCAACTCGAAAGAGTGGCACCGAGTCGGTGCTGT
GTGTCCCTCTGACAATGTGCTCTGGCCTGCAGAACAATCTCTTGACGCGGTTCTA
TCTAGTTACGCGTTAAACCAACTAGAAATTTTTT
VEGFA 1-15 del cr532 evopreQ1
(SEQ ID NO: 362)
GATGTCTGCAGGCCAGATGAGTTTAAGAGCTAAGCTGGAAACAGCATAGCAAGT
TTAAATAAGGCTAGTCCGTTATCAACGTGAAAACGTGACTCCGAGTCGGAGTTGT
GTGTCCCTCTGACAATGTGCTCTGGCCTGCAGAACAATCTCTTGACGCGGTTCTA
TCTAGTTACGCGTTAAACCAACTAGAAATTTTTT
VEGFA 1-15 del cr961 evopreQ1
(SEQ ID NO: 363)
GATGTCTGCAGGCCAGATGAGTTTAAGAGCTAAGCTGGAAACAGCATAGCAAGT
TTAAATAAGGCTAGTCCGTTATCAACTCGAAAGAGTGCAACCGAGTCGGTTGTGT
GTGTCCCTCTGACAATGTGCTCTGGCCTGCAGAACAATCTCTTGACGCGGTTCTA
TCTAGTTACGCGTTAAACCAACTAGAAATTTTTT
VEGFA 1-15 del flip and extension scaffold evopreQ1
(SEQ ID NO: 364)
GATGTCTGCAGGCCAGATGAGTTTAAGAGCTAAGCTGGAAACAGCATAGCAAGT
TTAAATAAGGCTAGTCCGTTATCAACTTGAAAAAGTGGCACCGAGTCGGTGCTGT
GTGTCCCTCTGACAATGTGCTCTGGCCTGCAGAACAATCTCTTGACGCGGTTCTA
TCTAGTTACGCGTTAAACCAACTAGAAATTTTTT
RUNX1 + 1FLAG standard scaffold evopreQ1
(SEQ ID NO: 365)
GCATTTTCAGGAGGAAGCGAGTTTTAGAGCTAGAAATAGCAAGTTAAAATAAGG
CTAGTCCGTTATCAACTTGAAAAAGTGGCACCGAGTCGGTGCTGTCTGAAGCCAT
CCCTTATCGTCGTCATCCTTGTAATCCTTCCTCCTGAAAATAACTCTCTTTGACGC
GGTTCTATCTAGTTACGCGTTAAACCAACTAGAAATTTTTT
RUNX1 + 1FLAG cr748 evopreQ1
(SEQ ID NO: 366)
GCATTTTCAGGAGGAAGCGAGTTTAAGAGCTAAGCTGGAAACAGCATAGCAAGT
TTAAATAAGGCTAGTCCGTTATCAACTCGAGAGAGTGGCACCGAGTCGGTGCTTG
TCTGAAGCCATCCCTTATCGTCGTCATCCTTGTAATCCTTCCTCCTGAAAATAACT
CTCTTTGACGCGGTTCTATCTAGTTACGCGTTAAACCAACTAGAAATTTTTT
RUNX1 + 1FLAG cr289 evopreQ1
(SEQ ID NO: 367)
GCATTTTCAGGAGGAAGCGAGTTTAAGAGCTAAGCTGGAAACAGCATAGCAAGT
TTAAATAAGGCTAGTCCGTTATCAACTGGAAACAGTGGCACCGAGTCGGTGCTTG
TCTGAAGCCATCCCTTATCGTCGTCATCCTTGTAATCCTTCCTCCTGAAAATAACT
CTCTTTGACGCGGTTCTATCTAGTTACGCGTTAAACCAACTAGAAATTTTTT
RUNX1 + 1FLAG cr622 evopreQ1
(SEQ ID NO: 368)
GCATTTTCAGGAGGAAGCGAGTTTAAGAGCTAAGCTGGAAACAGCATAGCAAGT
TTAAATAAGGCTAGTCCGTTATCAGCTGGAAACAGCGGCACCGAGTCGGTGCTT
GTCTGAAGCCATCCCTTATCGTCGTCATCCTTGTAATCCTTCCTCCTGAAAATAAC
TCTCTTTGACGCGGTTCTATCTAGTTACGCGTTAAACCAACTAGAAATTTTTT
RUNX1 + 1FLAG cr772 evopreQ1
(SEQ ID NO: 369)
GCATTTTCAGGAGGAAGCGAGTTTAAGAGCTAAGCTGGAAACAGCATAGCAAGT
TTAAATAAGGCTAGTCCGTTATCAACTCGAAAGAGTGGCACCGAGTCGGTGCTTG
TCTGAAGCCATCCCTTATCGTCGTCATCCTTGTAATCCTTCCTCCTGAAAATAACT
CTCTTTGACGCGGTTCTATCTAGTTACGCGTTAAACCAACTAGAAATTTTTT
RUNX1 + 1FLAG cr532 evopreQ1
(SEQ ID NO: 370)
GCATTTTCAGGAGGAAGCGAGTTTAAGAGCTAAGCTGGAAACAGCATAGCAAGT
TTAAATAAGGCTAGTCCGTTATCAACGTGAAAACGTGACTCCGAGTCGGAGTTTG
TCTGAAGCCATCCCTTATCGTCGTCATCCTTGTAATCCTTCCTCCTGAAAATAACT
CTCTTTGACGCGGTTCTATCTAGTTACGCGTTAAACCAACTAGAAATTTTTT
RUNX1 + 1FLAG cr961 evopreQ1
(SEQ ID NO: 371)
GCATTTTCAGGAGGAAGCGAGTTTAAGAGCTAAGCTGGAAACAGCATAGCAAGT
TTAAATAAGGCTAGTCCGTTATCAACTCGAAAGAGTGCAACCGAGTCGGTTGTTG
TCTGAAGCCATCCCTTATCGTCGTCATCCTTGTAATCCTTCCTCCTGAAAATAACT
CTCTTTGACGCGGTTCTATCTAGTTACGCGTTAAACCAACTAGAAATTTTTT
RUNX1 + 1FLAG flip and extension scaffold evopreQ1
(SEQ ID NO: 372)
GCATTTTCAGGAGGAAGCGAGTTTAAGAGCTAAGCTGGAAACAGCATAGCAAGT
TTAAATAAGGCTAGTCCGTTATCAACTTGAAAAAGTGGCACCGAGTCGGTGCTTG
TCTGAAGCCATCCCTTATCGTCGTCATCCTTGTAATCCTTCCTCCTGAAAATAACT
CTCTTTGACGCGGTTCTATCTAGTTACGCGTTAAACCAACTAGAAATTTTTT
DNMT1 + 1FLAG standard scaffold evopreQ1
(SEQ ID NO: 373)
GATTCCTGGTGCCAGAAACAGTTTTAGAGCTAGAAATAGCAAGTTAAAATAAGG
CTAGTCCGTTATCAACTTGAAAAAGTGGCACCGAGTCGGTGCTCTGCCCTCCCGT
CACCCCTGTCTTATCGTCGTCATCCTTGTAATCTTCTGGCACCAGGACCTCTTCTT
TGACGCGGTTCTATCTAGTTACGCGTTAAACCAACTAGAAATTTTTT
DNMT1 + 1FLAG cr748 evopreQ1
(SEQ ID NO: 374)
GATTCCTGGTGCCAGAAACAGTTTAAGAGCTAAGCTGGAAACAGCATAGCAAGT
TTAAATAAGGCTAGTCCGTTATCAACTCGAGAGAGTGGCACCGAGTCGGTGCTTC
TGCCCTCCCGTCACCCCTGTCTTATCGTCGTCATCCTTGTAATCTTCTGGCACCAG
GACCTCTTCTTTGACGCGGTTCTATCTAGTTACGCGTTAAACCAACTAGAAATTTT
TT
DNMT1 + 1FLAG cr289 evopreQ1
(SEQ ID NO: 375)
GATTCCTGGTGCCAGAAACAGTTTAAGAGCTAAGCTGGAAACAGCATAGCAAGT
TTAAATAAGGCTAGTCCGTTATCAACTGGAAACAGTGGCACCGAGTCGGTGCTTC
TGCCCTCCCGTCACCCCTGTCTTATCGTCGTCATCCTTGTAATCTTCTGGCACCAG
GACCTCTTCTTTGACGCGGTTCTATCTAGTTACGCGTTAAACCAACTAGAAATTTT
TT
DNMT1 + 1FLAG cr622 evopreQ1
(SEQ ID NO: 376)
GATTCCTGGTGCCAGAAACAGTTTAAGAGCTAAGCTGGAAACAGCATAGCAAGT
TTAAATAAGGCTAGTCCGTTATCAGCTGGAAACAGCGGCACCGAGTCGGTGCTTC
TGCCCTCCCGTCACCCCTGTCTTATCGTCGTCATCCTTGTAATCTTCTGGCACCAG
GACCTCTTCTTTGACGCGGTTCTATCTAGTTACGCGTTAAACCAACTAGAAATTTT
TT
DNMT1 + 1FLAG cr772 evopreQ1
(SEQ ID NO: 377)
GATTCCTGGTGCCAGAAACAGTTTAAGAGCTAAGCTGGAAACAGCATAGCAAGT
TTAAATAAGGCTAGTCCGTTATCAACTCGAAAGAGTGGCACCGAGTCGGTGCTTC
TGCCCTCCCGTCACCCCTGTCTTATCGTCGTCATCCTTGTAATCTTCTGGCACCAG
GACCTCTTCTTTGACGCGGTTCTATCTAGTTACGCGTTAAACCAACTAGAAATTTT
TT
DNMT1 + 1FLAG cr532 evopreQ1
(SEQ ID NO: 378)
GATTCCTGGTGCCAGAAACAGTTTAAGAGCTAAGCTGGAAACAGCATAGCAAGT
TTAAATAAGGCTAGTCCGTTATCAACGTGAAAACGTGACTCCGAGTCGGAGTTTC
TGCCCTCCCGTCACCCCTGTCTTATCGTCGTCATCCTTGTAATCTTCTGGCACCAG
GACCTCTTCTTTGACGCGGTTCTATCTAGTTACGCGTTAAACCAACTAGAAATTTT
TT
DNMT1 + 1FLAG cr961 evopreQ1
(SEQ ID NO: 379)
GATTCCTGGTGCCAGAAACAGTTTAAGAGCTAAGCTGGAAACAGCATAGCAAGT
TTAAATAAGGCTAGTCCGTTATCAACTCGAAAGAGTGCAACCGAGTCGGTTGTTC
TGCCCTCCCGTCACCCCTGTCTTATCGTCGTCATCCTTGTAATCTTCTGGCACCAG
GACCTCTTCTTTGACGCGGTTCTATCTAGTTACGCGTTAAACCAACTAGAAATTTT
TT
DNMT1 + 1FLAG flip and extension scaffold evopreQ1
(SEQ ID NO: 380)
GATTCCTGGTGCCAGAAACAGTTTAAGAGCTAAGCTGGAAACAGCATAGCAAGT
TTAAATAAGGCTAGTCCGTTATCAACTTGAAAAAGTGGCACCGAGTCGGTGCTTC
TGCCCTCCCGTCACCCCTGTCTTATCGTCGTCATCCTTGTAATCTTCTGGCACCAG
GACCTCTTCTTTGACGCGGTTCTATCTAGTTACGCGTTAAACCAACTAGAAATTTT
TT

The present disclosure contemplates any such ways to further improve the efficacy of the prime editing systems utilized in the methods and complexes disclosed herein.

In various embodiments, it may be advantageous to limit the appearance of a consecutive sequence of Ts from the extension arm, as consecutive series of T's may limit the capacity of the PEgRNA to be transcribed. For example, strings of at least three consecutive T's, at least four consecutive T's, at least five consecutive T's, at least six consecutive T's, at least seven consecutive T's, at least eight consecutive T's, at least nine consecutive T's, at least ten consecutive T's, at least eleven consecutive T's, at least twelve consecutive T's, at least thirteen consecutive T's, at least fourteen consecutive T's, or at least fifteen consecutive T's should be avoided when designing the PEgRNA, or should be at least removed from the final designed sequence. In one embodiment, one can avoid the inclusion of unwanted strings of consecutive T's in PEgRNA extension arms by avoiding target sites that are rich in consecutive A:T nucleobase pairs.

### Methods of Treatment and Uses

In some aspects, the present disclosure provides methods of treating trinucleotide repeat disorders, including Huntington's disease and Friedreich's ataxia, using prime editing.

In one aspect, the present disclosure provides methods of treating Huntington's disease by prime editing comprising contacting a target nucleotide sequence with any of the pegRNA-prime editor complexes or compositions disclosed herein. In some embodiments, the contacting results in the contraction or replacement of a CAG repeat sequence in the target nucleotide sequence. In some embodiments, the CAG repeat sequence is contracted from greater than 35 repeats to 35 or fewer repeats (e.g., 35 repeats, 34 repeats, 33 repeats, 32 repeats, 31 repeats, 30 repeats, 29 repeats, 28 repeats, 27 repeats, 26 repeats, 25 repeats, 24 repeats, 23 repeats, 22 repeats, 21 repeats, 20 repeats, 19 repeats, 18 repeats, 17 repeats, 16 repeats, 15 repeats, 14 repeats, 13 repeats, 12 repeats, 11 repeats, 10 repeats, 9 repeats, 8 repeats, 7 repeats, 6 repeats, 5 repeats, 4 repeats, or 3 repeats). In certain embodiments, the CAG repeat sequence is greater than 35 repeats in length and is replaced with a CAG repeat sequence of 35 or fewer repeats in length (e.g., 35 or fewer repeats, 34 or fewer repeats, 33 or fewer repeats, 32 or fewer repeats, 31 or fewer repeats, 30 or fewer repeats, 29 or fewer repeats, 28 or fewer repeats, 27 or fewer repeats, 26 or fewer repeats, 25 or fewer repeats, 24 or fewer repeats, 23 or fewer repeats, 22 or fewer repeats, 21 or fewer repeats, 20 or fewer repeats, 19 or fewer repeats, 18 or fewer repeats, 17 or fewer repeats, 16 or fewer repeats, 15 or fewer repeats, 14 or fewer repeats, 13 or fewer repeats, 12 or fewer repeats, 11 or fewer repeats, 10 or fewer repeats, 9 or fewer repeats, 8 or fewer repeats, 7 or fewer repeats, 6 or fewer repeats, 5 or fewer repeats, 4 or fewer repeats, or 3 or fewer repeats). In certain embodiments, the CAG repeat sequence is contracted to four repeats or replaced with a sequence comprising four CAG repeats.

In some embodiments, the methods further comprise nicking the non-PAM-containing strand of the target nucleotide sequence using a nicking gRNA. In some embodiments, the spacer of the nicking gRNA comprises the nucleotide sequence: GGCGGCTGAGGAAGCTGAGG (SEQ ID NO: 387); GGCGGCGGCGGCGGCGGTGG (SEQ ID NO: 385); GCTGCTGCTGCTGCTGCTGC (SEQ ID NO: 384); GAAGGACTTGAGGGACTCGA (SEQ ID NO: 393); GTGAGGAAGCTGAGGAGGCGG (SEQ ID NO: 394); GCTGTTGCTGCTGCTGCTGC (SEQ ID NO: 395); GCTGCTGCTGCTGCTGCTGGA (SEQ ID NO: 383); GCTGCTGCTGGAAGGACTTG (SEQ ID NO: 382); GGCCTTCATCAGCTTTTCC (SEQ ID NO: 396); GGCTTTCATCAGCTTTTCC (SEQ ID NO: 397); GGGACTCGAAGGCCTTCAT (SEQ ID NO: 398); GGAAGGACTTGAGGGACTCG (SEQ ID NO: 399); GCTGCTGCTGGAAGGACTT (SEQ ID NO: 400); GCTGCTGCTGCTGCTGCTGG (SEQ ID NO: 401); GCTGGAAGGACTTGAGGGACT (SEQ ID NO: 402); GCTGCTGCTGCTGGAAGGACT (SEQ ID NO: 403); CTGCTGCTGCTGCTGCTGGAA (SEQ ID NO: 404); GCTGCTGCTGCTGCTGCTGCT (SEQ ID NO: 405); GCTGCTGGAAGGACTTGAG (SEQ ID NO: 406); GCTGCTGGAAGGACTTCAG (SEQ ID NO: 407); GCTGCTGAAAGGACTTCAG (SEQ ID NO: 408); or GCTGTTGGAAGGACTTCAG (SEQ ID NO: 409), or a nucleotide sequence at least 80%, at least 85%, at least 90%, at least 95%, at least 96%, at least 97%, at least 98%, or at least 99% identical to any of these. In certain embodiments, the spacer of the nicking gRNA comprises the nucleotide sequence GCTGCTGCTGCTGCTGCTGC (SEQ ID NO: 384), or a nucleotide sequence at least 80%, at least 85%, at least 90%, at least 95%, at least 96%, at least 97%, at least 98%, or at least 99% identical to SEQ ID NO: 384. In certain embodiments, the spacer of the nicking gRNA comprises the nucleotide sequence GCTGCTGGAAGGACTTGAG (SEQ ID NO: 406), or a nucleotide sequence at least 80%, at least 85%, at least 90%, at least 95%, at least 96%, at least 97%, at least 98%, or at least 99% identical to SEQ ID NO: 406.

In some embodiments, the contacting is performed in a cell. In some embodiments, the cell is a eukaryotic cell. In certain embodiments, the cell is a human cell. In certain embodiments, the cell is in vitro. In certain embodiments, the cell is ex vivo. In some embodiments, the cell is in a subject. In certain embodiments, the subject is a human.

In another aspect, the present disclosure provides methods of treating Friedreich's ataxia by prime editing comprising contacting a target nucleotide sequence with any of the complexes provided herein. In some embodiments, the contacting results in the deletion of a GAA repeat sequence in the target nucleotide sequence. In some embodiments, the GAA repeat sequence deleted comprises greater than about 50 GAA repeats, greater than about 51 GAA repeats, greater than about 52 GAA repeats, greater than about 53 GAA repeats, greater than about 54 GAA repeats, greater than about 55 GAA repeats, greater than about 56 GAA repeats, greater than about 57 GAA repeats, greater than about 58 GAA repeats, greater than about 59 GAA repeats, greater than about 60 GAA repeats, greater than about 61 GAA repeats, greater than about 62 GAA repeats, greater than about 63 GAA repeats, greater than about 64 GAA repeats, greater than about 65 GAA repeats, greater than about 66 GAA repeats, greater than about 67 GAA repeats, greater than about 68 GAA repeats, greater than about 69 GAA repeats, greater than about 70 GAA repeats, greater than about 71 GAA repeats, greater than about 72 GAA repeats, greater than about 73 GAA repeats, greater than about 74 GAA repeats, greater than about 75 GAA repeats, greater than about 76 GAA repeats, greater than about 77 GAA repeats, greater than about 78 GAA repeats, greater than about 79 GAA repeats, or greater than about 80 GAA repeats. In certain embodiments, the GAA repeat sequence deleted comprises greater than 65 GAA repeats.

In some embodiments, the methods further comprise nicking the non-PAM-containing strand of the target nucleotide sequence using a nicking gRNA. In some embodiments, the spacer of the nicking gRNA comprises the nucleotide sequence: GTCCCAAAGTGCTGAGATTAT (SEQ ID NO: 410); GTGTATTTTTTAGTAGATACT (SEQ ID NO: 411); GATTCTCCTGCCGCAGCCTC (SEQ ID NO: 412); or GCGACACCACGCCCGGCTAAC (SEQ ID NO: 413), or a nucleotide sequence at least 80%, at least 85%, at least 90%, at least 95%, at least 96%, at least 97%, at least 98%, or at least 99% identical to any of these. In certain embodiments, the spacer of the nicking gRNA comprises the nucleotide sequence GTGTATTTTTTAGTAGATACT (SEQ ID NO: 411), or a nucleotide sequence at least 80%, at least 85%, at least 90%, at least 95%, at least 96%, at least 97%, at least 98%, or at least 99% identical to SEQ ID NO: 411. In certain embodiments, the spacer of the nicking gRNA comprises the nucleotide sequence GCGACACCACGCCCGGCTAAC (SEQ ID NO: 413), or a nucleotide sequence at least 80%, at least 85%, at least 90%, at least 95%, at least 96%, at least 97%, at least 98%, or at least 99% identical to SEQ ID NO: 413.

In some embodiments, the contacting is performed in a cell. In some embodiments, the cell is a eukaryotic cell. In certain embodiments, the cell is a human cell. In certain embodiments, the cell is a central nervous system cell. In certain embodiments, the cell is in vitro. In certain embodiments, the cell is ex vivo. In some embodiments, the cell is in a subject. In certain embodiments, the subject is a human. In certain embodiments, the central nervous system is targeted.

In some embodiments, one or more polynucleotides encoding any of the complexes provided herein are delivered to a cell. In certain embodiments, two polynucleotides encoding any of the complexes provided herein are delivered to a cell. In certain embodiments, the two polynucleotides comprise two halves of a prime editor comprising a split intein capable of reassembling into a prime editor molecule. In some embodiments, the one or more polynucleotides encoding a complex provided herein are delivered to a cell in one or more adeno-associated virus (AAV) particles. Delivery of prime editor complexes has been described, for example, in U.S. Provisional Application, U.S.S.N., 63/426,336, filed Nov. 17, 2022, U.S. Provisional Application, U.S.S.N., 63/491,013, filed Mar. 17, 2023, and Davis, J. R., et al. Nat. Biotechnol. 2023, each of which is incorporated herein by reference. In certain embodiments, the one or more polynucleotides encoding the complex are delivered to the cell in two AAV particles. In some embodiments, one or both of the AAV particles comprise AAV1, AAV2, AAV3, AAV4, AAV5, AAV6, AAV7, AAV8, or AAV9. In certain embodiments, one or both of the AAV particles comprise AAV9. In some embodiments, the AAV particles target central nervous system cells.

In some embodiments, a first and second AAV particle are delivered to a cell. In certain embodiments, the first AAV particle comprises a polynucleotide comprising the structure 5′-[inverted terminal repeat (ITR) sequence]-[promoter]-[napDNAbp N-terminal fragment]-[N-intein]-[terminator sequence]-[ITR sequence]-3′. In certain embodiments, the second AAV particle comprises a polynucleotide comprising the structure 5′-[ITR sequence]-[promoter]-[C-intein]-[napDNAbp C-terminal fragment]-[reverse transcriptase]-[terminator sequence]-[optional nicking gRNA]-[pegRNA]-[ITR]-3′. In certain embodiments, the split site within the napDNAbp is between amino acid residues 844 and 845 of a Cas9 (e.g., a Cas9 protein of SEQ ID NO: 6). In certain embodiments, the split site within the napDNAbp is between amino acid residues 1024 and 1025 of a Cas9 protein (e.g., a Cas9 protein of SEQ ID NO: 6).

napDNAbp

In various embodiments, the prime editors utilized in the complexes and methods described herein comprise a nucleic acid programmable DNA binding protein (napDNAbp).

In various embodiments, prime editors may include a napDNAbp domain having a wild type Cas9 sequence, including, for example the canonical Streptococcus pyogenes Cas9 sequence of SEQ ID NO: 6, shown as follows.

| Description | Sequence | SEQ ID NO: |
|---|---|---|
| SpCas9 | MDKKYSIGLDIGTNSVGWAVITDEYKVPSKKFKVLGN | 6 |
| Streptococcus | TDRHSIKKNLIGALLFDSGETAEATRLKRTARRRYTRR | |
| pyogenes | KNRICYLQEIFSNEMAKVDDSFFHRLEESFLVEEDKKH | |
| M1 | ERHPIFGNIVDEVAYHEKYPTIYHLRKKLVDSTDKADL | |
| SwissProt | RLIYLALAHMIKFRGHFLIEGDLNPDNSDVDKLFIQLVQ | |
| Accession | TYNQLFEENPINASGVDAKAILSARLSKSRRLENLIAQL | |
| No. | PGEKKNGLFGNLIALSLGLTPNFKSNFDLAEDAKLQLS | |
| Q99ZW2 | KDTYDDDLDNLLAQIGDQYADLFLAAKNLSDAILLSDI | |
| Wild type | LRVNTEITKAPLSASMIKRYDEHHQDLTLLKALVRQQL | |
| | PEKYKEIFFDQSKNGYAGYIDGGASQEEFYKFIKPILEK | |
| | MDGTEELLVKLNREDLLRKQRTFDNGSIPHQIHLGELH | |
| | AILRRQEDFYPFLKDNREKIEKILTFRIPYYVGPLARGN | |
| | SRFAWMTRKSEETITPWNFEEVVDKGASAQSFIERMTN | |
| | FDKNLPNEKVLPKHSLLYEYFTVYNELTKVKYVTEGM | |
| | RKPAFLSGEQKKAIVDLLFKTNRKVTVKQLKEDYFKKI | |
| | ECFDSVEISGVEDRFNASLGTYHDLLKIIKDKDFLDNEE | |
| | NEDILEDIVLTLTLFEDREMIEERLKTYAHLFDDKVMK | |
| | QLKRRRYTGWGRLSRKLINGIRDKQSGKTILDFLKSDG | |
| | FANRNFMQLIHDDSLTFKEDIQKAQVSGQGDSLHEHIA | |
| | NLAGSPAIKKGILQTVKVVDELVKVMGRHKPENIVIEM | |
| | ARENQTTQKGQKNSRERMKRIEEGIKELGSQILKEHPV | |
| | ENTQLQNEKLYLYYLQNGRDMYVDQELDINRLSDYD | |
| | VDHIVPQSFLKDDSIDNKVLTRSDKNRGKSDNVPSEEV | |
| | VKKMKNYWRQLLNAKLITQRKFDNLTKAERGGLSEL | |
| | DKAGFIKRQLVETRQITKHVAQILDSRMNTKYDENDK | |
| | LIREVKVITLKSKLVSDFRKDFQFYKVREINNYHHAHD | |
| | AYLNAVVGTALIKKYPKLESEFVYGDYKVYDVRKMIA | |
| | KSEQEIGKATAKYFFYSNIMNFFKTEITLANGEIRKRPLI | |
| | ETNGETGEIVWDKGRDFATVRKVLSMPQVNIVKKTEV | |
| | QTGGFSKESILPKRNSDKLIARKKDWDPKKYGGFDSPT | |
| | VAYSVLVVAKVEKGKSKKLKSVKELLGITIMERSSFEK | |
| | NPIDFLEAKGYKEVKKDLIIKLPKYSLFELENGRKRML | |
| | ASAGELQKGNELALPSKYVNFLYLASHYEKLKGSPED | |
| | NEQKQLFVEQHKHYLDEIIEQISEFSKRVILADANLDKV | |
| | LSAYNKHRDKPIREQAENIIHLFTLTNLGAPAAFKYFDT | |
| | TIDRKRYTSTKEVLDATLIHQSITGLYETRIDLSQLGGD | |

In other embodiments, the prime editors may include a napDNAbp domain having a modified Cas9 sequence, including, for example the nickase variant of Streptococcus pyogenes Cas9 of SEQ ID NO: 7 having an H840A substitution relative to the wild type SpCas9 (of SEQ ID NO: 6), shown as follows:

| | | |
|---|---|---|
| Cas9 nickase | MDKKYSIGLDIGTNSVGWAVITDEYKVPSKKFKVLGNTD | SEQ ID NO: 7 |
| Streptococcus | RHSIKKNLIGALLFDSGETAEATRLKRTARRRYTRRKNRI | |
| pyogenes | CYLQEIFSNEMAKVDDSFFHRLEESFLVEEDKKHERHPIF | |
| Q99ZW2 | GNIVDEVAYHEKYPTIYHLRKKLVDSTDKADLRLIYLAL | |
| Cas9 with | AHMIKFRGHFLIEGDLNPDNSDVDKLFIQLVQTYNQLFEE | |
| H840A | NPINASGVDAKAILSARLSKSRRLENLIAQLPGEKKNGLF | |
| | GNLIALSLGLTPNFKSNFDLAEDAKLQLSKDTYDDDLDN | |
| | LLAQIGDQYADLFLAAKNLSDAILLSDILRVNTEITKAPLS | |
| | ASMIKRYDEHHQDLTLLKALVRQQLPEKYKEIFFDQSKN | |
| | GYAGYIDGGASQEEFYKFIKPILEKMDGTEELLVKLNRE | |
| | DLLRKQRTFDNGSIPHQIHLGELHAILRRQEDFYPFLKDN | |
| | REKIEKILTFRIPYYVGPLARGNSRFAWMTRKSEETITPW | |
| | NFEEVVDKGASAQSFIERMTNFDKNLPNEKVLPKHSLLY | |
| | EYFTVYNELTKVKYVTEGMRKPAFLSGEQKKAIVDLLFK | |
| | TNRKVTVKQLKEDYFKKIECFDSVEISGVEDRFNASLGT | |
| | YHDLLKIIKDKDFLDNEENEDILEDIVLTLTLFEDREMIEE | |
| | RLKTYAHLFDDKVMKQLKRRRYTGWGRLSRKLINGIRD | |
| | KQSGKTILDFLKSDGFANRNFMQLIHDDSLTFKEDIQKAQ | |
| | VSGQGDSLHEHIANLAGSPAIKKGILQTVKVVDELVKVM | |
| | GRHKPENIVIEMARENQTTQKGQKNSRERMKRIEEGIKE | |
| | LGSQILKEHPVENTQLQNEKLYLYYLQNGRDMYVDQEL | |
| | DINRLSDYDVDAIVPQSFLKDDSIDNKVLTRSDKNRGKS | |
| | DNVPSEEVVKKMKNYWRQLLNAKLITQRKFDNLTKAER | |
| | GGLSELDKAGFIKRQLVETRQITKHVAQILDSRMNTKYD | |
| | ENDKLIREVKVITLKSKLVSDFRKDFQFYKVREINNYHH | |
| | AHDAYLNAVVGTALIKKYPKLESEFVYGDYKVYDVRK | |
| | MIAKSEQEIGKATAKYFFYSNIMNFFKTEITLANGEIRKRP | |
| | LIETNGETGEIVWDKGRDFATVRKVLSMPQVNIVKKTEV | |
| | QTGGFSKESILPKRNSDKLIARKKDWDPKKYGGFDSPTV | |
| | AYSVLVVAKVEKGKSKKLKSVKELLGITIMERSSFEKNPI | |
| | DFLEAKGYKEVKKDLIIKLPKYSLFELENGRKRMLASAG | |
| | ELQKGNELALPSKYVNFLYLASHYEKLKGSPEDNEQKQL | |
| | FVEQHKHYLDEIIEQISEFSKRVILADANLDKVLSAYNKH | |
| | RDKPIREQAENIIHLFTLTNLGAPAAFKYFDTTIDRKRYTS | |
| | TKEVLDATLIHQSITGLYETRIDLSQLGGD | |

The prime editors described herein may include any of the modified Cas9 sequences described above, or any variant thereof having at least 80%, at least 85%, at least 90%, at least 95%, or at least 99% sequence identity thereto. In some embodiments, the prime editors used in the methods described herein include any of the following other wild type SpCas9 sequences, which may be modified with one or more of the mutations described herein at corresponding amino acid positions:

| Description | Sequence |
|---|---|
| SpCas9 | ATGGATAAGAAATACTCAATAGGCTTAGATATCGGCACAAATAG |
| Streptococcus | CGTCGGATGGGCGGTGATCACTGATGATTATAAGGTTCCGTCTAA |
| pyogenes | AAAGTTCAAGGTTCTGGGAAATACAGACCGCCACAGTATCAAAA |
| MGAS1882 | AAAATCTTATAGGGGCTCTTTTATTTGGCAGTGGAGAGACAGCGG |
| wild type | AAGCGACTCGTCTCAAACGGACAGCTCGTAGAAGGTATACACGTC |
| NC_017053.1 | GGAAGAATCGTATTTGTTATCTACAGGAGATTTTTTCAAATGAGA |
| | TGGCGAAAGTAGATGATAGTTTCTTTCATCGACTTGAAGAGTCTT |
| | TTTTGGTGGAAGAAGACAAGAAGCATGAACGTCATCCTATTTTTG |
| | GAAATATAGTAGATGAAGTTGCTTATCATGAGAAATATCCAACTA |
| | TCTATCATCTGCGAAAAAAATTGGCAGATTCTACTGATAAAGCGG |
| | ATTTGCGCTTAATCTATTTGGCCTTAGCGCATATGATTAAGTTTCG |
| | TGGTCATTTTTTGATTGAGGGAGATTTAAATCCTGATAATAGTGAT |
| | GTGGACAAACTATTTATCCAGTTGGTACAAATCTACAATCAATTA |
| | TTTGAAGAAAACCCTATTAACGCAAGTAGAGTAGATGCTAAAGC |
| | GATTCTTTCTGCACGATTGAGTAAATCAAGACGATTAGAAAATCT |
| | CATTGCTCAGCTCCCCGGTGAGAAGAGAAATGGCTTGTTTGGGAA |
| | TCTCATTGCTTTGTCATTGGGATTGACCCCTAATTTTAAATCAAAT |
| | TTTGATTTGGCAGAAGATGCTAAATTACAGCTTTCAAAAGATACT |
| | TACGATGATGATTTAGATAATTTATTGGCGCAAATTGGAGATCAA |
| | TATGCTGATTTGTTTTTGGCAGCTAAGAATTTATCAGATGCTATTT |
| | TACTTTCAGATATCCTAAGAGTAAATAGTGAAATAACTAAGGCTC |
| | CCCTATCAGCTTCAATGATTAAGCGCTACGATGAACATCATCAAG |
| | ACTTGACTCTTTTAAAAGCTTTAGTTCGACAACAACTTCCAGAAA |
| | AGTATAAAGAAATCTTTTTTGATCAATCAAAAAACGGATATGCAG |
| | GTTATATTGATGGGGGAGCTAGCCAAGAAGAATTTTATAAATTTA |
| | TCAAACCAATTTTAGAAAAAATGGATGGTACTGAGGAATTATTGG |
| | TGAAACTAAATCGTGAAGATTTGCTGCGCAAGCAACGGACCTTTG |
| | ACAACGGCTCTATTCCCCATCAAATTCACTTGGGTGAGCTGCATG |
| | CTATTTTGAGAAGACAAGAAGACTTTTATCCATTTTTAAAAGACA |
| | ATCGTGAGAAGATTGAAAAAATCTTGACTTTTCGAATTCCTTATT |
| | ATGTTGGTCCATTGGCGCGTGGCAATAGTCGTTTTGCATGGATGA |
| | CTCGGAAGTCTGAAGAAACAATTACCCCATGGAATTTTGAAGAAG |
| | TTGTCGATAAAGGTGCTTCAGCTCAATCATTTATTGAACGCATGA |
| | CAAACTTTGATAAAAATCTTCCAAATGAAAAAGTACTACCAAAAC |
| | ATAGTTTGCTTTATGAGTATTTTACGGTTTATAACGAATTGACAAA |
| | GGTCAAATATGTTACTGAGGGAATGCGAAAACCAGCATTTCTTTC |
| | AGGTGAACAGAAGAAAGCCATTGTTGATTTACTCTTCAAAACAAA |
| | TCGAAAAGTAACCGTTAAGCAATTAAAAGAAGATTATTTCAAAA |
| | AAATAGAATGTTTTGATAGTGTTGAAATTTCAGGAGTTGAAGATA |
| | GATTTAATGCTTCATTAGGCGCCTACCATGATTTGCTAAAAATTAT |
| | TAAAGATAAAGATTTTTTGGATAATGAAGAAAATGAAGATATCTT |
| | AGAGGATATTGTTTTAACATTGACCTTATTTGAAGATAGGGGGAT |
| | GATTGAGGAAAGACTTAAAACATATGCTCACCTCTTTGATGATAA |
| | GGTGATGAAACAGCTTAAACGTCGCCGTTATACTGGTTGGGGACG |
| | TTTGTCTCGAAAATTGATTAATGGTATTAGGGATAAGCAATCTGG |
| | CAAAACAATATTAGATTTTTTGAAATCAGATGGTTTTGCCAATCG |
| | CAATTTTATGCAGCTGATCCATGATGATAGTTTGACATTTAAAGA |
| | AGATATTCAAAAAGCACAGGTGTCTGGACAAGGCCATAGTTTACA |
| | TGAACAGATTGCTAACTTAGCTGGCAGTCCTGCTATTAAAAAAGG |
| | TATTTTACAGACTGTAAAAATTGTTGATGAACTGGTCAAAGTAAT |
| | GGGGCATAAGCCAGAAAATATCGTTATTGAAATGGCACGTGAAA |
| | ATCAGACAACTCAAAAGGGCCAGAAAAATTCGCGAGAGCGTATG |
| | AAACGAATCGAAGAAGGTATCAAAGAATTAGGAAGTCAGATTCT |
| | TAAAGAGCATCCTGTTGAAAATACTCAATTGCAAAATGAAAAGCT |
| | CTATCTCTATTATCTACAAAATGGAAGAGACATGTATGTGGACCA |
| | AGAATTAGATATTAATCGTTTAAGTGATTATGATGTCGATCACAT |
| | TGTTCCACAAAGTTTCATTAAAGACGATTCAATAGACAATAAGGT |
| | ACTAACGCGTTCTGATAAAAATCGTGGTAAATCGGATAACGTTCC |
| | AAGTGAAGAAGTAGTCAAAAAGATGAAAAACTATTGGAGACAAC |
| | TTCTAAACGCCAAGTTAATCACTCAACGTAAGTTTGATAATTTAA |
| | CGAAAGCTGAACGTGGAGGTTTGAGTGAACTTGATAAAGCTGGTT |
| | TTATCAAACGCCAATTGGTTGAAACTCGCCAAATCACTAAGCATG |
| | TGGCACAAATTTTGGATAGTCGCATGAATACTAAATACGATGAAA |
| | ATGATAAACTTATTCGAGAGGTTAAAGTGATTACCTTAAAATCTA |
| | AATTAGTTTCTGACTTCCGAAAAGATTTCCAATTCTATAAAGTAC |
| | GTGAGATTAACAATTACCATCATGCCCATGATGCGTATCTAAATG |
| | CCGTCGTTGGAACTGCTTTGATTAAGAAATATCCAAAACTTGAAT |
| | CGGAGTTTGTCTATGGTGATTATAAAGTTTATGATGTTCGTAAAAT |
| | GATTGCTAAGTCTGAGCAAGAAATAGGCAAAGCAACCGCAAAAT |
| | ATTTCTTTTACTCTAATATCATGAACTTCTTCAAAACAGAAATTAC |
| | ACTTGCAAATGGAGAGATTCGCAAACGCCCTCTAATCGAAACTAA |
| | TGGGGAAACTGGAGAAATTGTCTGGGATAAAGGGCGAGATTTTG |
| | CCACAGTGCGCAAAGTATTGTCCATGCCCCAAGTCAATATTGTCA |
| | AGAAAACAGAAGTACAGACAGGCGGATTCTCCAAGGAGTCAATT |
| | TTACCAAAAAGAAATTCGGACAAGCTTATTGCTCGTAAAAAAGAC |
| | TGGGATCCAAAAAAATATGGTGGTTTTGATAGTCCAACGGTAGCT |
| | TATTCAGTCCTAGTGGTTGCTAAGGTGGAAAAAGGGAAATCGAA |
| | GAAGTTAAAATCCGTTAAAGAGTTACTAGGGATCACAATTATGGA |
| | AAGAAGTTCCTTTGAAAAAAATCCGATTGACTTTTTAGAAGCTAA |
| | AGGATATAAGGAAGTTAAAAAAGACTTAATCATTAAACTACCTA |
| | AATATAGTCTTTTTGAGTTAGAAAACGGTCGTAAACGGATGCTGG |
| | CTAGTGCCGGAGAATTACAAAAAGGAAATGAGCTGGCTCTGCCA |
| | AGCAAATATGTGAATTTTTTATATTTAGCTAGTCATTATGAAAAGT |
| | TGAAGGGTAGTCCAGAAGATAACGAACAAAAACAATTGTTTGTG |
| | GAGCAGCATAAGCATTATTTAGATGAGATTATTGAGCAAATCAGT |
| | GAATTTTCTAAGCGTGTTATTTTAGCAGATGCCAATTTAGATAAA |
| | GTTCTTAGTGCATATAACAAACATAGAGACAAACCAATACGTGAA |
| | CAAGCAGAAAATATTATTCATTTATTTACGTTGACGAATCTTGGA |
| | GCTCCCGCTGCTTTTAAATATTTTGATACAACAATTGATCGTAAAC |
| | GATATACGTCTACAAAAGAAGTTTTAGATGCCACTCTTATCCATC |
| | AATCCATCACTGGTCTTTATGAAACACGCATTGATTTGAGTCAGC |
| | TAGGAGGTGACTGA (SEQ ID NO: 25) |
| SpCas9 | MDKKYSIGLDIGTNSVGWAVITDDYKVPSKKFKVLGNTDRHSIKKN |
| Streptococcus | LIGALLFGSGETAEATRLKRTARRRYTRRKNRICYLQEIFSNEMAKV |
| pyogenes | DDSFFHRLEESFLVEEDKKHERHPIFGNIVDEVAYHEKYPTIYHLRKK |
| MGAS1882 | LADSTDKADLRLIYLALAHMIKFRGHFLIEGDLNPDNSDVDKLFIQLV |
| wild type | QIYNQLFEENPINASRVDAKAILSARLSKSRRLENLIAQLPGEKRNGL |
| NC_017053.1 | FGNLIALSLGLTPNFKSNFDLAEDAKLQLSKDTYDDDLDNLLAQIGD |
| | QYADLFLAAKNLSDAILLSDILRVNSEITKAPLSASMIKRYDEHHQDL |
| | TLLKALVRQQLPEKYKEIFFDQSKNGYAGYIDGGASQEEFYKFIKPIL |
| | EKMDGTEELLVKLNREDLLRKQRTFDNGSIPHQIHLGELHAILRRQE |
| | DFYPFLKDNREKIEKILTFRIPYYVGPLARGNSRFAWMTRKSEETITP |
| | WNFEEVVDKGASAQSFIERMTNFDKNLPNEKVLPKHSLLYEYFTVY |
| | NELTKVKYVTEGMRKPAFLSGEQKKAIVDLLFKTNRKVTVKQLKED |
| | YFKKIECFDSVEISGVEDRFNASLGAYHDLLKIIKDKDFLDNEENEDIL |
| | EDIVLTLTLFEDRGMIEERLKTYAHLFDDKVMKQLKRRRYTGWGRL |
| | SRKLINGIRDKQSGKTILDFLKSDGFANRNFMQLIHDDSLTFKEDIQK |
| | AQVSGQGHSLHEQIANLAGSPAIKKGILQTVKIVDELVKVMGHKPEN |
| | IVIEMARENQTTQKGQKNSRERMKRIEEGIKELGSQILKEHPVENTQL |
| | QNEKLYLYYLQNGRDMYVDQELDINRLSDYDVDHIVPQSFIKDDSID |
| | NKVLTRSDKNRGKSDNVPSEEVVKKMKNYWRQLLNAKLITQRKFD |
| | NLTKAERGGLSELDKAGFIKRQLVETRQITKHVAQILDSRMNTKYDE |
| | NDKLIREVKVITLKSKLVSDFRKDFQFYKVREINNYHHAHDAYLNA |
| | VVGTALIKKYPKLESEFVYGDYKVYDVRKMIAKSEQEIGKATAKYF |
| | FYSNIMNFFKTEITLANGEIRKRPLIETNGETGEIVWDKGRDFATVRK |
| | VLSMPQVNIVKKTEVQTGGFSKESILPKRNSDKLIARKKDWDPKKYG |
| | GFDSPTVAYSVLVVAKVEKGKSKKLKSVKELLGITIMERSSFEKNPID |
| | FLEAKGYKEVKKDLIIKLPKYSLFELENGRKRMLASAGELQKGNELA |
| | LPSKYVNFLYLASHYEKLKGSPEDNEQKQLFVEQHKHYLDEIIEQISE |
| | FSKRVILADANLDKVLSAYNKHRDKPIREQAENIIHLFTLTNLGAPAA |
| | FKYFDTTIDRKRYTSTKEVLDATLIHQSITGLYETRIDLSQLGGD (SEQ |
| | ID NO: 8) |
| SpCas9 | ATGGATAAAAAGTATTCTATTGGTTTAGACATCGGCACTAATTCC |
| Streptococcus | GTTGGATGGGCTGTCATAACCGATGAATACAAAGTACCTTCAAAG |
| pyogenes wild | AAATTTAAGGTGTTGGGGAACACAGACCGTCATTCGATTAAAAAG |
| type | AATCTTATCGGTGCCCTCCTATTCGATAGTGGCGAAACGGCAGAG |
| SWBC2D7W014 | GCGACTCGCCTGAAACGAACCGCTCGGAGAAGGTATACACGTCG |
| | CAAGAACCGAATATGTTACTTACAAGAAATTTTTAGCAATGAGAT |
| | GGCCAAAGTTGACGATTCTTTCTTTCACCGTTTGGAAGAGTCCTTC |
| | CTTGTCGAAGAGGACAAGAAACATGAACGGCACCCCATCTTTGG |
| | AAACATAGTAGATGAGGTGGCATATCATGAAAAGTACCCAACGA |
| | TTTATCACCTCAGAAAAAAGCTAGTTGACTCAACTGATAAAGCGG |
| | ACCTGAGGTTAATCTACTTGGCTCTTGCCCATATGATAAAGTTCCG |
| | TGGGCACTTTCTCATTGAGGGTGATCTAAATCCGGACAACTCGGA |
| | TGTCGACAAACTGTTCATCCAGTTAGTACAAACCTATAATCAGTT |
| | GTTTGAAGAGAACCCTATAAATGCAAGTGGCGTGGATGCGAAGG |
| | CTATTCTTAGCGCCCGCCTCTCTAAATCCCGACGGCTAGAAAACC |
| | TGATCGCACAATTACCCGGAGAGAAGAAAAATGGGTTGTTCGGT |
| | AACCTTATAGCGCTCTCACTAGGCCTGACACCAAATTTTAAGTCG |
| | AACTTCGACTTAGCTGAAGATGCCAAATTGCAGCTTAGTAAGGAC |
| | ACGTACGATGACGATCTCGACAATCTACTGGCACAAATTGGAGAT |
| | CAGTATGCGGACTTATTTTTGGCTGCCAAAAACCTTAGCGATGCA |
| | ATCCTCCTATCTGACATACTGAGAGTTAATACTGAGATTACCAAG |
| | GCGCCGTTATCCGCTTCAATGATCAAAAGGTACGATGAACATCAC |
| | CAAGACTTGACACTTCTCAAGGCCCTAGTCCGTCAGCAACTGCCT |
| | GAGAAATATAAGGAAATATTCTTTGATCAGTCGAAAAACGGGTA |
| | CGCAGGTTATATTGACGGCGGAGCGAGTCAAGAGGAATTCTACA |
| | AGTTTATCAAACCCATATTAGAGAAGATGGATGGGACGGAAGAG |
| | TTGCTTGTAAAACTCAATCGCGAAGATCTACTGCGAAAGCAGCGG |
| | ACTTTCGACAACGGTAGCATTCCACATCAAATCCACTTAGGCGAA |
| | TTGCATGCTATACTTAGAAGGCAGGAGGATTTTTATCCGTTCCTCA |
| | AAGACAATCGTGAAAAGATTGAGAAAATCCTAACCTTTCGCATAC |
| | CTTACTATGTGGGACCCCTGGCCCGAGGGAACTCTCGGTTCGCAT |
| | GGATGACAAGAAAGTCCGAAGAAACGATTACTCCATGGAATTTT |
| | GAGGAAGTTGTCGATAAAGGTGCGTCAGCTCAATCGTTCATCGAG |
| | AGGATGACCAACTTTGACAAGAATTTACCGAACGAAAAAGTATT |
| | GCCTAAGCACAGTTTACTTTACGAGTATTTCACAGTGTACAATGA |
| | ACTCACGAAAGTTAAGTATGTCACTGAGGGCATGCGTAAACCCGC |
| | CTTTCTAAGCGGAGAACAGAAGAAAGCAATAGTAGATCTGTTATT |
| | CAAGACCAACCGCAAAGTGACAGTTAAGCAATTGAAAGAGGACT |
| | ACTTTAAGAAAATTGAATGCTTCGATTCTGTCGAGATCTCCGGGG |
| | TAGAAGATCGATTTAATGCGTCACTTGGTACGTATCATGACCTCC |
| | TAAAGATAATTAAAGATAAGGACTTCCTGGATAACGAAGAGAAT |
| | GAAGATATCTTAGAAGATATAGTGTTGACTCTTACCCTCTTTGAA |
| | GATCGGGAAATGATTGAGGAAAGACTAAAAACATACGCTCACCT |
| | GTTCGACGATAAGGTTATGAAACAGTTAAAGAGGCGTCGCTATAC |
| | GGGCTGGGGACGATTGTCGCGGAAACTTATCAACGGGATAAGAG |
| | ACAAGCAAAGTGGTAAAACTATTCTCGATTTTCTAAAGAGCGACG |
| | GCTTCGCCAATAGGAACTTTATGCAGCTGATCCATGATGACTCTTT |
| | AACCTTCAAAGAGGATATACAAAAGGCACAGGTTTCCGGACAAG |
| | GGGACTCATTGCACGAACATATTGCGAATCTTGCTGGTTCGCCAG |
| | CCATCAAAAAGGGCATACTCCAGACAGTCAAAGTAGTGGATGAG |
| | CTAGTTAAGGTCATGGGACGTCACAAACCGGAAAACATTGTAATC |
| | GAGATGGCACGCGAAAATCAAACGACTCAGAAGGGGCAAAAAA |
| | ACAGTCGAGAGCGGATGAAGAGAATAGAAGAGGGTATTAAAGAA |
| | CTGGGCAGCCAGATCTTAAAGGAGCATCCTGTGGAAAATACCCA |
| | ATTGCAGAACGAGAAACTTTACCTCTATTACCTACAAAATGGAAG |
| | GGACATGTATGTTGATCAGGAACTGGACATAAACCGTTTATCTGA |
| | TTACGACGTCGATCACATTGTACCCCAATCCTTTTTGAAGGACGA |
| | TTCAATCGACAATAAAGTGCTTACACGCTCGGATAAGAACCGAGG |
| | GAAAAGTGACAATGTTCCAAGCGAGGAAGTCGTAAAGAAAATGA |
| | AGAACTATTGGCGGCAGCTCCTAAATGCGAAACTGATAACGCAA |
| | AGAAAGTTCGATAACTTAACTAAAGCTGAGAGGGGTGGCTTGTCT |
| | GAACTTGACAAGGCCGGATTTATTAAACGTCAGCTCGTGGAAACC |
| | CGCCAAATCACAAAGCATGTTGCACAGATACTAGATTCCCGAATG |
| | AATACGAAATACGACGAGAACGATAAGCTGATTCGGGAAGTCAA |
| | AGTAATCACTTTAAAGTCAAAATTGGTGTCGGACTTCAGAAAGGA |
| | TTTTCAATTCTATAAAGTTAGGGAGATAAATAACTACCACCATGC |
| | GCACGACGCTTATCTTAATGCCGTCGTAGGGACCGCACTCATTAA |
| | GAAATACCCGAAGCTAGAAAGTGAGTTTGTGTATGGTGATTACAA |
| | AGTTTATGACGTCCGTAAGATGATCGCGAAAAGCGAACAGGAGA |
| | TAGGCAAGGCTACAGCCAAATACTTCTTTTATTCTAACATTATGA |
| | ATTTCTTTAAGACGGAAATCACTCTGGCAAACGGAGAGATACGCA |
| | AACGACCTTTAATTGAAACCAATGGGGAGACAGGTGAAATCGTA |
| | TGGGATAAGGGCCGGGACTTCGCGACGGTGAGAAAAGTTTTGTCC |
| | ATGCCCCAAGTCAACATAGTAAAGAAAACTGAGGTGCAGACCGG |
| | AGGGTTTTCAAAGGAATCGATTCTTCCAAAAAGGAATAGTGATAA |
| | GCTCATCGCTCGTAAAAAGGACTGGGACCCGAAAAAGTACGGTG |
| | GCTTCGATAGCCCTACAGTTGCCTATTCTGTCCTAGTAGTGGCAA |
| | AAGTTGAGAAGGGAAAATCCAAGAAACTGAAGTCAGTCAAAGAA |
| | TTATTGGGGATAACGATTATGGAGCGCTCGTCTTTTGAAAAGAAC |
| | CCCATCGACTTCCTTGAGGCGAAAGGTTACAAGGAAGTAAAAAA |
| | GGATCTCATAATTAAACTACCAAAGTATAGTCTGTTTGAGTTAGA |
| | AAATGGCCGAAAACGGATGTTGGCTAGCGCCGGAGAGCTTCAAA |
| | AGGGGAACGAACTCGCACTACCGTCTAAATACGTGAATTTCCTGT |
| | ATTTAGCGTCCCATTACGAGAAGTTGAAAGGTTCACCTGAAGATA |
| | ACGAACAGAAGCAACTTTTTGTTGAGCAGCACAAACATTATCTCG |
| | ACGAAATCATAGAGCAAATTTCGGAATTCAGTAAGAGAGTCATCC |
| | TAGCTGATGCCAATCTGGACAAAGTATTAAGCGCATACAACAAGC |
| | ACAGGGATAAACCCATACGTGAGCAGGCGGAAAATATTATCCAT |
| | TTGTTTACTCTTACCAACCTCGGCGCTCCAGCCGCATTCAAGTATT |
| | TTGACACAACGATAGATCGCAAACGATACACTTCTACCAAGGAG |
| | GTGCTAGACGCGACACTGATTCACCAATCCATCACGGGATTATAT |
| | GAAACTCGGATAGATTTGTCACAGCTTGGGGGTGACGGATCCCCC |
| | AAGAAGAAGAGGAAAGTCTCGAGCGACTACAAAGACCATGACGG |
| | TGATTATAAAGATCATGACATCGATTACAAGGATGACGATGACAA |
| | GGCTGCAGGA (SEQ ID NO: 26) |
| SpCas9 | MDKKYSIGLDIGTNSVGWAVITDEYKVPSKKFKVLGNTDRHSIKKNL |
| Streptococcus | IGALLFDSGETAEATRLKRTARRRYTRRKNRICYLQEIFSNEMAKVD |
| pyogenes wild | DSFFHRLEESFLVEEDKKHERHPIFGNIVDEVAYHEKYPTIYHLRKKL |
| type | VDSTDKADLRLIYLALAHMIKFRGHFLIEGDLNPDNSDVDKLFIQLV |
| Encoded | QTYNQLFEENPINASGVDAKAILSARLSKSRRLENLIAQLPGEKKNGL |
| product of | FGNLIALSLGLTPNFKSNFDLAEDAKLQLSKDTYDDDLDNLLAQIGD |
| SWBC2D7W014 | QYADLFLAAKNLSDAILLSDILRVNTEITKAPLSASMIKRYDEHHQDL |
| | TLLKALVRQQLPEKYKEIFFDQSKNGYAGYIDGGASQEEFYKFIKPIL |
| | EKMDGTEELLVKLNREDLLRKQRTFDNGSIPHQIHLGELHAILRRQE |
| | DFYPFLKDNREKIEKILTFRIPYYVGPLARGNSRFAWMTRKSEETITP |
| | WNFEEVVDKGASAQSFIERMTNFDKNLPNEKVLPKHSLLYEYFTVY |
| | NELTKVKYVTEGMRKPAFLSGEQKKAIVDLLFKTNRKVTVKQLKED |
| | YFKKIECFDSVEISGVEDRFNASLGTYHDLLKIIKDKDFLDNEENEDIL |
| | EDIVLTLTLFEDREMIEERLKTYAHLFDDKVMKQLKRRRYTGWGRL |
| | SRKLINGIRDKQSGKTILDFLKSDGFANRNFMQLIHDDSLTFKEDIQK |
| | AQVSGQGDSLHEHIANLAGSPAIKKGILQTVKVVDELVKVMGRHKP |
| | ENIVIEMARENQTTQKGQKNSRERMKRIEEGIKELGSQILKEHPVENT |
| | QLQNEKLYLYYLQNGRDMYVDQELDINRLSDYDVDHIVPQSFLKDD |
| | SIDNKVLTRSDKNRGKSDNVPSEEVVKKMKNYWRQLLNAKLITQRK |
| | FDNLTKAERGGLSELDKAGFIKRQLVETRQITKHVAQILDSRMNTKY |
| | DENDKLIREVKVITLKSKLVSDFRKDFQFYKVREINNYHHAHDAYLN |
| | AVVGTALIKKYPKLESEFVYGDYKVYDVRKMIAKSEQEIGKATAKY |
| | FFYSNIMNFFKTEITLANGEIRKRPLIETNGETGEIVWDKGRDFATVR |
| | KVLSMPQVNIVKKTEVQTGGFSKESILPKRNSDKLIARKKDWDPKKY |
| | GGFDSPTVAYSVLVVAKVEKGKSKKLKSVKELLGITIMERSSFEKNPI |
| | DFLEAKGYKEVKKDLIIKLPKYSLFELENGRKRMLASAGELQKGNEL |
| | ALPSKYVNFLYLASHYEKLKGSPEDNEQKQLFVEQHKHYLDEIIEQIS |
| | EFSKRVILADANLDKVLSAYNKHRDKPIREQAENIIHLFTLTNLGAPA |
| | AFKYFDTTIDRKRYTSTKEVLDATLIHQSITGLYETRIDLSQLGGDGSP |
| | KKKRKVSSDYKDHDGDYKDHDIDYKDDDDKAAG (SEQ ID NO: 9) |
| SpCas9 | ATGGATAAGAAATACTCAATAGGCTTAGATATCGGCACAAATAG |
| Streptococcus | CGTCGGATGGGCGGTGATCACTGATGAATATAAGGTTCCGTCTAA |
| pyogenes | AAAGTTCAAGGTTCTGGGAAATACAGACCGCCACAGTATCAAAA |
| M1GAS wild | AAAATCTTATAGGGGCTCTTTTATTTGACAGTGGAGAGACAGCGG |
| type | AAGCGACTCGTCTCAAACGGACAGCTCGTAGAAGGTATACACGTC |
| NC_002737.2 | GGAAGAATCGTATTTGTTATCTACAGGAGATTTTTTCAAATGAGA |
| | TGGCGAAAGTAGATGATAGTTTCTTTCATCGACTTGAAGAGTCTT |
| | TTTTGGTGGAAGAAGACAAGAAGCATGAACGTCATCCTATTTTTG |
| | GAAATATAGTAGATGAAGTTGCTTATCATGAGAAATATCCAACTA |
| | TCTATCATCTGCGAAAAAAATTGGTAGATTCTACTGATAAAGCGG |
| | ATTTGCGCTTAATCTATTTGGCCTTAGCGCATATGATTAAGTTTCG |
| | TGGTCATTTTTTGATTGAGGGAGATTTAAATCCTGATAATAGTGAT |
| | GTGGACAAACTATTTATCCAGTTGGTACAAACCTACAATCAATTA |
| | TTTGAAGAAAACCCTATTAACGCAAGTGGAGTAGATGCTAAAGC |
| | GATTCTTTCTGCACGATTGAGTAAATCAAGACGATTAGAAAATCT |
| | CATTGCTCAGCTCCCCGGTGAGAAGAAAAATGGCTTATTTGGGAA |
| | TCTCATTGCTTTGTCATTGGGTTTGACCCCTAATTTTAAATCAAAT |
| | TTTGATTTGGCAGAAGATGCTAAATTACAGCTTTCAAAAGATACT |
| | TACGATGATGATTTAGATAATTTATTGGCGCAAATTGGAGATCAA |
| | TATGCTGATTTGTTTTTGGCAGCTAAGAATTTATCAGATGCTATTT |
| | TACTTTCAGATATCCTAAGAGTAAATACTGAAATAACTAAGGCTC |
| | CCCTATCAGCTTCAATGATTAAACGCTACGATGAACATCATCAAG |
| | ACTTGACTCTTTTAAAAGCTTTAGTTCGACAACAACTTCCAGAAA |
| | AGTATAAAGAAATCTTTTTTGATCAATCAAAAAACGGATATGCAG |
| | GTTATATTGATGGGGGAGCTAGCCAAGAAGAATTTTATAAATTTA |
| | TCAAACCAATTTTAGAAAAAATGGATGGTACTGAGGAATTATTGG |
| | TGAAACTAAATCGTGAAGATTTGCTGCGCAAGCAACGGACCTTTG |
| | ACAACGGCTCTATTCCCCATCAAATTCACTTGGGTGAGCTGCATG |
| | CTATTTTGAGAAGACAAGAAGACTTTTATCCATTTTTAAAAGACA |
| | ATCGTGAGAAGATTGAAAAAATCTTGACTTTTCGAATTCCTTATT |
| | ATGTTGGTCCATTGGCGCGTGGCAATAGTCGTTTTGCATGGATGA |
| | CTCGGAAGTCTGAAGAAACAATTACCCCATGGAATTTTGAAGAAG |
| | TTGTCGATAAAGGTGCTTCAGCTCAATCATTTATTGAACGCATGA |
| | CAAACTTTGATAAAAATCTTCCAAATGAAAAAGTACTACCAAAAC |
| | ATAGTTTGCTTTATGAGTATTTTACGGTTTATAACGAATTGACAAA |
| | GGTCAAATATGTTACTGAAGGAATGCGAAAACCAGCATTTCTTTC |
| | AGGTGAACAGAAGAAAGCCATTGTTGATTTACTCTTCAAAACAAA |
| | TCGAAAAGTAACCGTTAAGCAATTAAAAGAAGATTATTTCAAAA |
| | AAATAGAATGTTTTGATAGTGTTGAAATTTCAGGAGTTGAAGATA |
| | GATTTAATGCTTCATTAGGTACCTACCATGATTTGCTAAAAATTAT |
| | TAAAGATAAAGATTTTTTGGATAATGAAGAAAATGAAGATATCTT |
| | AGAGGATATTGTTTTAACATTGACCTTATTTGAAGATAGGGAGAT |
| | GATTGAGGAAAGACTTAAAACATATGCTCACCTCTTTGATGATAA |
| | GGTGATGAAACAGCTTAAACGTCGCCGTTATACTGGTTGGGGACG |
| | TTTGTCTCGAAAATTGATTAATGGTATTAGGGATAAGCAATCTGG |
| | CAAAACAATATTAGATTTTTTGAAATCAGATGGTTTTGCCAATCG |
| | CAATTTTATGCAGCTGATCCATGATGATAGTTTGACATTTAAAGA |
| | AGACATTCAAAAAGCACAAGTGTCTGGACAAGGCGATAGTTTAC |
| | ATGAACATATTGCAAATTTAGCTGGTAGCCCTGCTATTAAAAAAG |
| | GTATTTTACAGACTGTAAAAGTTGTTGATGAATTGGTCAAAGTAA |
| | TGGGGCGGCATAAGCCAGAAAATATCGTTATTGAAATGGCACGT |
| | GAAAATCAGACAACTCAAAAGGGCCAGAAAAATTCGCGAGAGCG |
| | TATGAAACGAATCGAAGAAGGTATCAAAGAATTAGGAAGTCAGA |
| | TTCTTAAAGAGCATCCTGTTGAAAATACTCAATTGCAAAATGAAA |
| | AGCTCTATCTCTATTATCTCCAAAATGGAAGAGACATGTATGTGG |
| | ACCAAGAATTAGATATTAATCGTTTAAGTGATTATGATGTCGATC |
| | ACATTGTTCCACAAAGTTTCCTTAAAGACGATTCAATAGACAATA |
| | AGGTCTTAACGCGTTCTGATAAAAATCGTGGTAAATCGGATAACG |
| | TTCCAAGTGAAGAAGTAGTCAAAAAGATGAAAAACTATTGGAGA |
| | CAACTTCTAAACGCCAAGTTAATCACTCAACGTAAGTTTGATAAT |
| | TTAACGAAAGCTGAACGTGGAGGTTTGAGTGAACTTGATAAAGCT |
| | GGTTTTATCAAACGCCAATTGGTTGAAACTCGCCAAATCACTAAG |
| | CATGTGGCACAAATTTTGGATAGTCGCATGAATACTAAATACGAT |
| | GAAAATGATAAACTTATTCGAGAGGTTAAAGTGATTACCTTAAAA |
| | TCTAAATTAGTTTCTGACTTCCGAAAAGATTTCCAATTCTATAAAG |
| | TACGTGAGATTAACAATTACCATCATGCCCATGATGCGTATCTAA |
| | ATGCCGTCGTTGGAACTGCTTTGATTAAGAAATATCCAAAACTTG |
| | AATCGGAGTTTGTCTATGGTGATTATAAAGTTTATGATGTTCGTAA |
| | AATGATTGCTAAGTCTGAGCAAGAAATAGGCAAAGCAACCGCAA |
| | AATATTTCTTTTACTCTAATATCATGAACTTCTTCAAAACAGAAAT |
| | TACACTTGCAAATGGAGAGATTCGCAAACGCCCTCTAATCGAAAC |
| | TAATGGGGAAACTGGAGAAATTGTCTGGGATAAAGGGCGAGATT |
| | TTGCCACAGTGCGCAAAGTATTGTCCATGCCCCAAGTCAATATTG |
| | TCAAGAAAACAGAAGTACAGACAGGCGGATTCTCCAAGGAGTCA |
| | ATTTTACCAAAAAGAAATTCGGACAAGCTTATTGCTCGTAAAAAA |
| | GACTGGGATCCAAAAAAATATGGTGGTTTTGATAGTCCAACGGTA |
| | GCTTATTCAGTCCTAGTGGTTGCTAAGGTGGAAAAAGGGAAATCG |
| | AAGAAGTTAAAATCCGTTAAAGAGTTACTAGGGATCACAATTATG |
| | GAAAGAAGTTCCTTTGAAAAAAATCCGATTGACTTTTTAGAAGCT |
| | AAAGGATATAAGGAAGTTAAAAAAGACTTAATCATTAAACTACC |
| | TAAATATAGTCTTTTTGAGTTAGAAAACGGTCGTAAACGGATGCT |
| | GGCTAGTGCCGGAGAATTACAAAAAGGAAATGAGCTGGCTCTGC |
| | CAAGCAAATATGTGAATTTTTTATATTTAGCTAGTCATTATGAAA |
| | AGTTGAAGGGTAGTCCAGAAGATAACGAACAAAAACAATTGTTT |
| | GTGGAGCAGCATAAGCATTATTTAGATGAGATTATTGAGCAAATC |
| | AGTGAATTTTCTAAGCGTGTTATTTTAGCAGATGCCAATTTAGATA |
| | AAGTTCTTAGTGCATATAACAAACATAGAGACAAACCAATACGTG |
| | AACAAGCAGAAAATATTATTCATTTATTTACGTTGACGAATCTTG |
| | GAGCTCCCGCTGCTTTTAAATATTTTGATACAACAATTGATCGTAA |
| | ACGATATACGTCTACAAAAGAAGTTTTAGATGCCACTCTTATCCA |
| | TCAATCCATCACTGGTCTTTATGAAACACGCATTGATTTGAGTCA |
| | GCTAGGAGGTGACTGA (SEQ ID NO: 27) |
| SpCas9 | MDKKYSIGLDIGTNSVGWAVITDEYKVPSKKFKVLGNTDRHSIKKNL |
| Streptococcus | IGALLFDSGETAEATRLKRTARRRYTRRKNRICYLQEIFSNEMAKVD |
| pyogenes | DSFFHRLEESFLVEEDKKHERHPIFGNIVDEVAYHEKYPTIYHLRKKL |
| M1GAS wild | VDSTDKADLRLIYLALAHMIKFRGHFLIEGDLNPDNSDVDKLFIQLV |
| type | QTYNQLFEENPINASGVDAKAILSARLSKSRRLENLIAQLPGEKKNGL |
| Encoded | FGNLIALSLGLTPNFKSNFDLAEDAKLQLSKDTYDDDLDNLLAQIGD |
| product of | QYADLFLAAKNLSDAILLSDILRVNTEITKAPLSASMIKRYDEHHQDL |
| NC_002737.2 | TLLKALVRQQLPEKYKEIFFDQSKNGYAGYIDGGASQEEFYKFIKPIL |
| (100% identical | EKMDGTEELLVKLNREDLLRKQRTFDNGSIPHQIHLGELHAILRRQE |
| to the canonical | DFYPFLKDNREKIEKILTFRIPYYVGPLARGNSRFAWMTRKSEETITP |
| Q99ZW2 | WNFEEVVDKGASAQSFIERMTNFDKNLPNEKVLPKHSLLYEYFTVY |
| wild type) | NELTKVKYVTEGMRKPAFLSGEQKKAIVDLLFKTNRKVTVKQLKED |
| | YFKKIECFDSVEISGVEDRFNASLGTYHDLLKIIKDKDFLDNEENEDIL |
| | EDIVLTLTLFEDREMIEERLKTYAHLFDDKVMKQLKRRRYTGWGRL |
| | SRKLINGIRDKQSGKTILDFLKSDGFANRNFMQLIHDDSLTFKEDIQK |
| | AQVSGQGDSLHEHIANLAGSPAIKKGILQTVKVVDELVKVMGRHKP |
| | ENIVIEMARENQTTQKGQKNSRERMKRIEEGIKELGSQILKEHPVENT |
| | QLQNEKLYLYYLQNGRDMYVDQELDINRLSDYDVDHIVPQSFLKDD |
| | SIDNKVLTRSDKNRGKSDNVPSEEVVKKMKNYWRQLLNAKLITQRK |
| | FDNLTKAERGGLSELDKAGFIKRQLVETRQITKHVAQILDSRMNTKY |
| | DENDKLIREVKVITLKSKLVSDFRKDFQFYKVREINNYHHAHDAYLN |
| | AVVGTALIKKYPKLESEFVYGDYKVYDVRKMIAKSEQEIGKATAKY |
| | FFYSNIMNFFKTEITLANGEIRKRPLIETNGETGEIVWDKGRDFATVR |
| | KVLSMPQVNIVKKTEVQTGGFSKESILPKRNSDKLIARKKDWDPKKY |
| | GGFDSPTVAYSVLVVAKVEKGKSKKLKSVKELLGITIMERSSFEKNPI |
| | DFLEAKGYKEVKKDLIIKLPKYSLFELENGRKRMLASAGELQKGNEL |
| | ALPSKYVNFLYLASHYEKLKGSPEDNEQKQLFVEQHKHYLDEIIEQIS |
| | EFSKRVILADANLDKVLSAYNKHRDKPIREQAENIIHLFTLTNLGAPA |
| | AFKYFDTTIDRKRYTSTKEVLDATLIHQSITGLYETRIDLSQLGGD |
| | (SEQ ID NO: 6) |

The prime editors used in the methods described herein may include any of the above SpCas9 sequences, or any variant thereof having at least 80%, at least 85%, at least 90%, at least 95%, or at least 99% sequence identity thereto. In other embodiments, the Cas9 protein can be a wild type Cas9 ortholog from another bacterial species different from the canonical Cas9 from S. pyogenes. For example, modified versions of the following Cas9 orthologs can be used in connection with the prime editors described in this specification by making mutations at positions corresponding to H840A or any other amino acids of interest in wild type SpCas9. In addition, any variant Cas9 orthologs having at least 80%, at least 85%, at least 90%, at least 95%, or at least 99% sequence identity to any of the below orthologs may also be used with the prime editors.

| Description | Sequence |
|---|---|
| LfCas9 | MKEYHIGLDIGTSSIGWAVTDSQFKLMRIKGKTAIGVRLFEEGKTAAERR |
| Lactobacillus | TFRTTRRRLKRRKWRLHYLDEIFAPHLQEVDENFLRRLKQSNIHPEDPTK |
| fermentum | NQAFIGKLLFPDLLKKNERGYPTLIKMRDELPVEQRAHYPVMNIYKLRE |
| wild type | AMINEDRQFDLREVYLAVHHIVKYRGHFLNNASVDKFKVGRIDFDKSFN |
| GenBank: | VLNEAYEELQNGEGSFTIEPSKVEKIGQLLLDTKMRKLDRQKAVAKLLE |
| SNX31424.1 1 | VKVADKEETKRNKQIATAMSKLVLGYKADFATVAMANGNEWKIDLSS |
| | ETSEDEIEKFREELSDAQNDILTEITSLFSQIMLNEIVPNGMSISESMMDRY |
| | WTHERQLAEVKEYLATQPASARKEFDQVYNKYIGQAPKERGFDLEKGL |
| | KKILSKKENWKEIDELLKAGDFLPKQRTSANGVIPHQMHQQELDRIIEKQ |
| | AKYYPWLATENPATGERDRHQAKYELDQLVSFRIPYYVGPLVTPEVQK |
| | ATSGAKFAWAKRKEDGEITPWNLWDKIDRAESAEAFIKRMTVKDTYLL |
| | NEDVLPANSLLYQKYNVLNELNNVRVNGRRLSVGIKQDIYTELFKKKKT |
| | VKASDVASLVMAKTRGVNKPSVEGLSDPKKFNSNLATYLDLKSIVGDK |
| | VDDNRYQTDLENIIEWRSVFEDGEIFADKLTEVEWLTDEQRSALVKKRY |
| | KGWGRLSKKLLTGIVDENGQRIIDLMWNTDQNFKEIVDQPVFKEQIDQL |
| | NQKAITNDGMTLRERVESVLDDAYTSPQNKKAIWQVVRVVEDIVKAVG |
| | NAPKSISIEFARNEGNKGEITRSRRTQLQKLFEDQAHELVKDTSLTEELEK |
| | APDLSDRYYFYFTQGGKDMYTGDPINFDEISTKYDIDHILPQSFVKDNSL |
| | DNRVLTSRKENNKKSDQVPAKLYAAKMKPYWNQLLKQGLITQRKFEN |
| | LTKDVDQNIKYRSLGFVKRQLVETRQVIKLTANILGSMYQEAGTEIIETR |
| | AGLTKQLREEFDLPKVREVNDYHHAVDAYLTTFAGQYLNRRYPKLRSF |
| | FVYGEYMKFKHGSDLKLRNFNFFHELMEGDKSQGKVVDQQTGELITTR |
| | DEVAKSFDRLLNMKYMLVSKEVHDRSDQLYGATIVTAKESGKLTSPIEI |
| | KKNRLVDLYGAYTNGTSAFMTIIKFTGNKPKYKVIGIPTTSAASLKRAGK |
| | PGSESYNQELHRIIKSNPKVKKGFEIVVPHVSYGQLIVDGDCKFTLASPTV |
| | QHPATQLVLSKKSLETISSGYKILKDKPAIANERLIRVFDEVVGQMNRYF |
| | TIFDQRSNRQKVADARDKFLSLPTESKYEGAKKVQVGKTEVITNLLMGL |
| | HANATQGDLKVLGLATFGFFQSTTGLSLSEDTMIVYQSPTGLFERRICLK |
| | DI (SEQ ID NO: 12) |
| SaCas9 | MDKKYSIGLDIGTNSVGWAVITDEYKVPSKKFKVLGNTDRHSIKKNLIG |
| Staphylococcus | ALLFDSGETAEATRLKRTARRRYTRRKNRICYLQEIFSNEMAKVDDSFFH |
| aureus | RLEESFLVEEDKKHERHPIFGNIVDEVAYHEKYPTIYHLRKKLVDSTDKA |
| wild type | DLRLIYLALAHMIKFRGHFLIEGDLNPDNSDVDKLFIQLVQTYNQLFEEN |
| GenBank: | PINASGVDAKAILSARLSKSRRLENLIAQLPGEKKNGLFGNLIALSLGLTP |
| AYD60528.1 | NFKSNFDLAEDAKLQLSKDTYDDDLDNLLAQIGDQYADLFLAAKNLSD |
| | AILLSDILRVNTEITKAPLSASMIKRYDEHHQDLTLLKALVRQQLPEKYK |
| | EIFFDQSKNGYAGYIDGGASQEEFYKFIKPILEKMDGTEELLVKLNREDL |
| | LRKQRTFDNGSIPHQIHLGELHAILRRQEDFYPFLKDNREKIEKILTFRIPY |
| | YVGPLARGNSRFAWMTRKSEETITPWNFEEVVDKGASAQSFIERMTNFD |
| | KNLPNEKVLPKHSLLYEYFTVYNELTKVKYVTEGMRKPAFLSGEQKKAI |
| | VDLLFKTNRKVTVKQLKEDYFKKIECFDSVEISGVEDRFNASLGTYHDL |
| | LKIIKDKDFLDNEENEDILEDIVLTLTLFEDREMIEERLKTYAHLFDDKVM |
| | KQLKRRRYTGWGRLSRKLINGIRDKQSGKTILDFLKSDGFANRNFMQLI |
| | HDDSLTFKEDIQKAQVSGQGDSLHEHIANLAGSPAIKKGILQTVKVVDEL |
| | VKVMGRHKPENIVIEMARENQTTQKGQKNSRERMKRIEEGIKELGSQIL |
| | KEHPVENTQLQNEKLYLYYLQNGRDMYVDQELDINRLSDYDVDHIVPQ |
| | SFLKDDSIDNKVLTRSDKNRGKSDNVPSEEVVKKMKNYWRQLLNAKLI |
| | TQRKFDNLTKAERGGLSELDKAGFIKRQLVETRQITKHVAQILDSRMNT |
| | KYDENDKLIREVKVITLKSKLVSDFRKDFQFYKVREINNYHHAHDAYLN |
| | AVVGTALIKKYPKLESEFVYGDYKVYDVRKMIAKSEQEIGKATAKYFFY |
| | SNIMNFFKTEITLANGEIRKRPLIETNGETGEIVWDKGRDFATVRKVLSM |
| | PQVNIVKKTEVQTGGFSKESILPKRNSDKLIARKKDWDPKKYGGFDSPT |
| | VAYSVLVVAKVEKGKSKKLKSVKELLGITIMERSSFEKNPIDFLEAKGYK |
| | EVKKDLIIKLPKYSLFELENGRKRMLASAGELQKGNELALPSKYVNFLY |
| | LASHYEKLKGSPEDNEQKQLFVEQHKHYLDEIIEQISEFSKRVILADANL |
| | DKVLSAYNKHRDKPIREQAENIIHLFTLTNLGAPAAFKYFDTTIDRKRYT |
| | STKEVLDATLIHQSITGLYETRIDLSQLGGD (SEQ ID NO: 11) |
| SaCas9 | MGKRNYILGLDIGITSVGYGIIDYETRDVIDAGVRLFKEANVENNEGRRS |
| Staphylococcus | KRGARRLKRRRRHRIQRVKKLLFDYNLLTDHSELSGINPYEARVKGLSQ |
| aureus | KLSEEEFSAALLHLAKRRGVHNVNEVEEDTGNELSTKEQISRNSKALEE |
| | KYVAELQLERLKKDGEVRGSINRFKTSDYVKEAKQLLKVQKAYHQLDQ |
| | SFIDTYIDLLETRRTYYEGPGEGSPFGWKDIKEWYEMLMGHCTYFPEELR |
| | SVKYAYNADLYNALNDLNNLVITRDENEKLEYYEKFQIIENVFKQKKKP |
| | TLKQIAKEILVNEEDIKGYRVTSTGKPEFTNLKVYHDIKDITARKEIIENA |
| | ELLDQIAKILTIYQSSEDIQEELTNLNSELTQEEIEQISNLKGYTGTHNLSL |
| | KAINLILDELWHTNDNQIAIFNRLKLVPKKVDLSQQKEIPTTLVDDFILSP |
| | VVKRSFIQSIKVINAIIKKYGLPNDIIIELAREKNSKDAQKMINEMQKRNR |
| | QTNERIEEIIRTTGKENAKYLIEKIKLHDMQEGKCLYSLEAIPLEDLLNNP |
| | FNYEVDHIIPRSVSFDNSFNNKVLVKQEENSKKGNRTPFQYLSSSDSKISY |
| | ETFKKHILNLAKGKGRISKTKKEYLLEERDINRFSVQKDFINRNLVDTRY |
| | ATRGLMNLLRSYFRVNNLDVKVKSINGGFTSFLRRKWKFKKERNKGYK |
| | HHAEDALIIANADFIFKEWKKLDKAKKVMENQMFEEKQAESMPEIETEQ |
| | EYKEIFITPHQIKHIKDFKDYKYSHRVDKKPNRKLINDTLYSTRKDDKGN |
| | TLIVNNLNGLYDKDNDKLKKLINKSPEKLLMYHHDPQTYQKLKLIMEQ |
| | YGDEKNPLYKYYEETGNYLTKYSKKDNGPVIKKIKYYGNKLNAHLDIT |
| | DDYPNSRNKVVKLSLKPYRFDVYLDNGVYKFVTVKNLDVIKKENYYEV |
| | NSKCYEEAKKLKKISNQAEFIASFYKNDLIKINGELYRVIGVNNDLLNRIE |
| | VNMIDITYREYLENMNDKRPPHIIKTIASKTQSIKKYSTDILGNLYEVKSK |
| | KHPQIIKK (SEQ ID NO: 13) |
| StCas9 | MLFNKCIIISINLDFSNKEKCMTKPYSIGLDIGTNSVGWAVITDNYKVPSK |
| Streptococcus | KMKVLGNTSKKYIKKNLLGVLLFDSGITAEGRRLKRTARRRYTRRRNRI |
| thermophilus | LYLQEIFSTEMATLDDAFFQRLDDSFLVPDDKRDSKYPIFGNLVEEKVYH |
| UniProtKB/ | DEFPTIYHLRKYLADSTKKADLRLVYLALAHMIKYRGHFLIEGEFNSKN |
| Swiss-Prot: | NDIQKNFQDFLDTYNAIFESDLSLENSKQLEEIVKDKISKLEKKDRILKLF |
| G3ECR1.2 | PGEKNSGIFSEFLKLIVGNQADFRKCFNLDEKASLHESKESYDEDLETLL |
| Wild type | GYIGDDYSDVFLKAKKLYDAILLSGFLTVTDNETEAPLSSAMIKRYNEH |
| | KEDLALLKEYIRNISLKTYNEVFKDDTKNGYAGYIDGKTNQEDFYVYLK |
| | NLLAEFEGADYFLEKIDREDFLRKQRTFDNGSIPYQIHLQEMRAILDKQA |
| | KFYPFLAKNKERIEKILTFRIPYYVGPLARGNSDFAWSIRKRNEKITPWNF |
| | EDVIDKESSAEAFINRMTSFDLYLPEEKVLPKHSLLYETFNVYNELTKVR |
| | FIAESMRDYQFLDSKQKKDIVRLYFKDKRKVTDKDIIEYLHAIYGYDGIE |
| | LKGIEKQFNSSLSTYHDLLNIINDKEFLDDSSNEAIIEEIIHTLTIFEDREMIK |
| | QRLSKFENIFDKSVLKKLSRRHYTGWGKLSAKLINGIRDEKSGNTILDYLI |
| | DDGISNRNFMQLIHDDALSFKKKIQKAQIIGDEDKGNIKEVVKSLPGSPAI |
| | KKGILQSIKIVDELVKVMGGRKPESIVVEMARENQYTNQGKSNSQQRLK |
| | RLEKSLKELGSKILKENIPAKLSKIDNNALQNDRLYLYYLQNGKDMYTG |
| | DDLDIDRLSNYDIDHIIPQAFLKDNSIDNKVLVSSASNRGKSDDFPSLEVV |
| | KKRKTFWYQLLKSKLISQRKFDNLTKAERGGLLPEDKAGFIQRQLVETR |
| | QITKHVARLLDEKFNNKKDENNRAVRTVKIITLKSTLVSQFRKDFELYK |
| | VREINDFHHAHDAYLNAVIASALLKKYPKLEPEFVYGDYPKYNSFRERK |
| | SATEKVYFYSNIMNIFKKSISLADGRVIERPLIEVNEETGESVWNKESDLA |
| | TVRRVLSYPQVNVVKKVEEQNHGLDRGKPKGLFNANLSSKPKPNSNEN |
| | LVGAKEYLDPKKYGGYAGISNSFAVLVKGTIEKGAKKKITNVLEFQGISI |
| | LDRINYRKDKLNFLLEKGYKDIELIIELPKYSLFELSDGSRRMLASILSTN |
| | NKRGEIHKGNQIFLSQKFVKLLYHAKRISNTINENHRKYVENHKKEFEEL |
| | FYYILEFNENYVGAKKNGKLLNSAFQSWQNHSIDELCSSFIGPTGSERKG |
| | LFELTSRGSAADFEFLGVKIPRYRDYTPSSLLKDATLIHQSVTGLYETRID |
| | LAKLGEG (SEQ ID NO: 14) |
| LcCas9 | MKIKNYNLALTPSTSAVGHVEVDDDLNILEPVHHQKAIGVAKFGEGETA |
| Lactobacillus | EARRLARSARRTTKRRANRINHYFNEIMKPEIDKVDPLMFDRIKQAGLSP |
| crispatus | LDERKEFRTVIFDRPNIASYYHNQFPTIWHLQKYLMITDEKADIRLIYWA |
| NCBI | LHSLLKHRGHFFNTTPMSQFKPGKLNLKDDMLALDDYNDLEGLSFAVA |
| Reference | NSPEIEKVIKDRSMHKKEKIAELKKLIVNDVPDKDLAKRNNKIITQIVNAI |
| Sequence: | MGNSFHLNFIFDMDLDKLTSKAWSFKLDDPELDTKFDAISGSMTDNQIGI |
| WP_133478044.1 | FETLQKIYSAISLLDILNGSSNVVDAKNALYDKHKRDLNLYFKFLNTLPD |
| Wild type | EIAKTLKAGYTLYIGNRKKDLLAARKLLKVNVAKNFSQDDFYKLINKEL |
| | KSIDKQGLQTRFSEKVGELVAQNNFLPVQRSSDNVFIPYQLNAITFNKILE |
| | NQGKYYDFLVKPNPAKKDRKNAPYELSQLMQFTIPYYVGPLVTPEEQV |
| | KSGIPKTSRFAWMVRKDNGAITPWNFYDKVDIEATADKFIKRSIAKDSY |
| | LLSELVLPKHSLLYEKYEVFNELSNVSLDGKKLSGGVKQILFNEVFKKTN |
| | KVNTSRILKALAKHNIPGSKITGLSNPEEFTSSLQTYNAWKKYFPNQIDNF |
| | AYQQDLEKMIEWSTVFEDHKILAKKLDEIEWLDDDQKKFVANTRLRGW |
| | GRLSKRLLTGLKDNYGKSIMQRLETTKANFQQIVYKPEFREQIDKISQAA |
| | AKNQSLEDILANSYTSPSNRKAIRKTMSVVDEYIKLNHGKEPDKIFLMFQ |
| | RSEQEKGKQTEARSKQLNRILSQLKADKSANKLESKQLADEFSNAIKKS |
| | KYKLNDKQYFYFQQLGRDALTGEVIDYDELYKYTVLHIIPRSKLTDDSQ |
| | NNKVLTKYKIVDGSVALKFGNSYSDALGMPIKAFWTELNRLKLIPKGKL |
| | LNLTTDESTLNKYQRDGYIARQLVETQQIVKLLATIMQSRFKHTKIIEVR |
| | NSQVANIRYQFDYFRIKNLNEYYRGFDAYLAAVVGTYLYKVYPKARRL |
| | FVYGQYLKPKKTNQENQDMHLDSEKKSQGFNFLWNLLYGKQDQIFVN |
| | GTDVIAFNRKDLITKMNTVYNYKSQKISLAIDYHNGAMFKATLFPRNDR |
| | DTAKTRKLIPKKKDYDTDIYGGYTSNVDGYMLLAEIIKRDGNKQYGFYG |
| | VPSRLVSELDTLKKTRYTEYEEKLKEIIKPELGVDLKKIKKIKILKNKVPF |
| | NQVIIDKGSKFFITSTSYRWNYRQLILSAESQQTLMDLVVDPDFSNHKAR |
| | KDARKNADERLIKVYEEILYQVKNYMPMFVELHRCYEKLVDAQKTFKS |
| | LKISDKAMVLNQILILLHSNATSPVLEKLGYHTRFTLGKKHNLISENAVL |
| | VTQSITGLKENHVSIKQML (SEQ ID NO: 15) |
| PdCas9 | MTNEKYSIGLDIGTSSIGFAVVNDNNRVIRVKGKNAIGVRLFDEGKAAA |
| Pedicoccus | DRRSFRTTRRSFRTTRRRLSRRRWRLKLLREIFDAYITPVDEAFFIRLKES |
| damnosus | NLSPKDSKKQYSGDILFNDRSDKDFYEKYPTIYHLRNALMTEHRKFDVR |
| NCBI | EIYLAIHHIMKFRGHFLNATPANNFKVGRLNLEEKFEELNDIYQRVFPDE |
| Reference | SIEFRTDNLEQIKEVLLDNKRSRADRQRTLVSDIYQSSEDKDIEKRNKAV |
| Sequence: | ATEILKASLGNKAKLNVITNVEVDKEAAKEWSITFDSESIDDDLAKIEGQ |
| WP_062913273.1 | MTDDGHEIIEVLRSLYSGITLSAIVPENHTLSQSMVAKYDLHKDHLKLFK |
| Wild type | KLINGMTDTKKAKNLRAAYDGYIDGVKGKVLPQEDFYKQVQVNLDDS |
| | AEANEIQTYIDQDIFMPKQRTKANGSIPHQLQQQELDQIIENQKAYYPWL |
| | AELNPNPDKKRQQLAKYKLDELVTFRVPYYVGPMITAKDQKNQSGAEF |
| | AWMIRKEPGNITPWNFDQKVDRMATANQFIKRMTTTDTYLLGEDVLPA |
| | QSLLYQKFEVLNELNKIRIDHKPISIEQKQQIFNDLFKQFKNVTIKHLQDY |
| | LVSQGQYSKRPLIEGLADEKRENSSLSTYSDLCGIFGAKLVEENDRQEDL |
| | EKIIEWSTIFEDKKIYRAKLNDLTWLTDDQKEKLATKRYQGWGRLSRKL |
| | LVGLKNSEHRNIMDILWITNENFMQIQAEPDFAKLVTDANKGMLEKTDS |
| | QDVINDLYTSPQNKKAIRQILLVVHDIQNAMHGQAPAKIHVEFARGEER |
| | NPRRSVQRQRQVEAAYEKVSNELVSAKVRQEFKEAINNKRDFKDRLFL |
| | YFMQGGIDIYTGKQLNIDQLSSYQIDHILPQAFVKDDSLTNRVLTNENQV |
| | KADSVPIDIFGKKMLSVWGRMKDQGLISKGKYRNLTMNPENISAHTENG |
| | FINRQLVETRQVIKLAVNILADEYGDSTQIISVKADLSHQMREDFELLKN |
| | RDVNDYHHAFDAYLAAFIGNYLLKRYPKLESYFVYGDFKKFTQKETKM |
| | RRFNFIYDLKHCDQVVNKETGEILWTKDEDIKYIRHLFAYKKILVSHEVR |
| | EKRGALYNQTIYKAKDDKGSGQESKKLIRIKDDKETKIYGGYSGKSLAY |
| | MTIVQITKKNKVSYRVIGIPTLALARLNKLENDSTENNGELYKIIKPQFTH |
| | YKVDKKNGEIIETTDDFKIVVSKVRFQQLIDDAGQFFMLASDTYKNNAQ |
| | QLVISNNALKAINNTNITDCPRDDLERLDNLRLDSAFDEIVKKMDKYFSA |
| | YDANNFREKIRNSNLIFYQLPVEDQWENNKITELGKRTVLTRILQGLHAN |
| | ATTTDMSIFKIKTPFGQLRQRSGISLSENAQLIYQSPTGLFERRVQLNKIK |
| | (SEQ ID NO: 16) |
| FnCas9 | MKKQKFSDYYLGFDIGTNSVGWCVTDLDYNVLRFNKKDMWGSRLFEE |
| Fusobaterium | AKTAAERRVQRNSRRRLKRRKWRLNLLEEIFSNEILKIDSNFFRRLKESSL |
| nucleatum | WLEDKSSKEKFTLFNDDNYKDYDFYKQYPTIFHLRNELIKNPEKKDIRLV |
| NCBI | YLAIHSIFKSRGHFLFEGQNLKEIKNFETLYNNLIAFLEDNGINKIIDKNNI |
| Reference | EKLEKIVCDSKKGLKDKEKEFKEIFNSDKQLVAIFKLSVGSSVSLNDLFD |
| Sequence: | TDEYKKGEVEKEKISFREQIYEDDKPIYYSILGEKIELLDIAKTFYDFMVL |
| WP_060798984.1 | NNILADSQYISEAKVKLYEEHKKDLKNLKYIIRKYNKGNYDKLFKDKNE |
| | NNYSAYIGLNKEKSKKEVIEKSRLKIDDLIKNIKGYLPKVEEIEEKDKAIF |
| | NKILNKIELKTILPKQRISDNGTLPYQIHEAELEKILENQSKYYDFLNYEE |
| | NGIITKDKLLMTFKFRIPYYVGPLNSYHKDKGGNSWIVRKEEGKILPWNF |
| | EQKVDIEKSAEEFIKRMTNKCTYLNGEDVIPKDTFLYSEYVILNELNKVQ |
| | VNDEFLNEENKRKIIDELFKENKKVSEKKFKEYLLVKQIVDGTIELKGVK |
| | DSFNSNYISYIRFKDIFGEKLNLDIYKEISEKSILWKCLYGDDKKIFEKKIK |
| | NEYGDILTKDEIKKINTFKFNNWGRLSEKLLTGIEFINLETGECYSSVMDA |
| | LRRTNYNLMELLSSKFTLQESINNENKEMNEASYRDLIEESYVSPSLKRAI |
| | FQTLKIYEEIRKITGRVPKKVFIEMARGGDESMKNKKIPARQEQLKKLYD |
| | SCGNDIANFSIDIKEMKNSLISYDNNSLRQKKLYLYYLQFGKCMYTGREI |
| | DLDRLLQNNDTYDIDHIYPRSKVIKDDSFDNLVLVLKNENAEKSNEYPV |
| | KKEIQEKMKSFWRFLKEKNFISDEKYKRLTGKDDFELRGFMARQLVNV |
| | RQTTKEVGKILQQIEPEIKIVYSKAEIASSFREMFDFIKVRELNDTHHAKD |
| | AYLNIVAGNVYNTKFTEKPYRYLQEIKENYDVKKIYNYDIKNAWDKEN |
| | SLEIVKKNMEKNTVNITRFIKEKKGQLFDLNPIKKGETSNEIISIKPKVYN |
| | GKDDKLNEKYGYYKSLNPAYFLYVEHKEKNKRIKSFERVNLVDVNNIK |
| | DEKSLVKYLIENKKLVEPRVIKKVYKRQVILINDYPYSIVTLDSNKLMDF |
| | ENLKPLFLENKYEKILKNVIKFLEDNQGKSEENYKFIYLKKKDRYEKNET |
| | LESVKDRYNLEFNEMYDKFLEKLDSKDYKNYMNNKKYQELLDVKEKFI |
| | KLNLFDKAFTLKSFLDLFNRKTMADFSKVGLTKYLGKIQKISSNVLSKNE |
| | LYLLEESVTGLFVKKIKL (SEQ ID NO: 17) |
| EcCas9 | RRKQRIQILQELLGEEVLKTDPGFFHRMKESRYVVEDKRTLDGKQVELP |
| Enterococcus | YALFVDKDYTDKEYYKQFPTINHLIVYLMTTSDTPDIRLVYLALHYYMK |
| cecorum | NRGNFLHSGDINNVKDINDILEQLDNVLETFLDGWNLKLKSYVEDIKNIY |
| NCBI | NRDLGRGERKKAFVNTLGAKTKAEKAFCSLISGGSTNLAELFDDSSLKEI |
| Reference | ETPKIEFASSSLEDKIDGIQEALEDRFAVIEAAKRLYDWKTLTDILGDSSS |
| Sequence: | LAEARVNSYQMHHEQLLELKSLVKEYLDRKVFQEVFVSLNVANNYPAY |
| WP_047338501.1 | IGHTKINGKKKELEVKRTKRNDFYSYVKKQVIEPIKKKVSDEAVLTKLSE |
| Wild type | IESLIEVDKYLPLQVNSDNGVIPYQVKLNELTRIFDNLENRIPVLRENRDK |
| | IIKTFKFRIPYYVGSLNGVVKNGKCTNWMVRKEEGKIYPWNFEDKVDLE |
| | ASAEQFIRRMTNKCTYLVNEDVLPKYSLLYSKYLVLSELNNLRIDGRPLD |
| | VKIKQDIYENVFKKNRKVTLKKIKKYLLKEGIITDDDELSGLADDVKSSL |
| | TAYRDFKEKLGHLDLSEAQMENIILNITLFGDDKKLLKKRLAALYPFIDD |
| | KSLNRIATLNYRDWGRLSERFLSGITSVDQETGELRTIIQCMYETQANLM |
| | QLLAEPYHFVEAIEKENPKVDLESISYRIVNDLYVSPAVKRQIWQTLLVIK |
| | DIKQVMKHDPERIFIEMAREKQESKKTKSRKQVLSEVYKKAKEYEHLFE |
| | KLNSLTEEQLRSKKIYLYFTQLGKCMYSGEPIDFENLVSANSNYDIDHIYP |
| | QSKTIDDSFNNIVLVKKSLNAYKSNHYPIDKNIRDNEKVKTLWNTLVSK |
| | GLITKEKYERLIRSTPFSDEELAGFIARQLVETRQSTKAVAEILSNWFPESE |
| | IVYSKAKNVSNFRQDFEILKVRELNDCHHAHDAYLNIVVGNAYHTKFTN |
| | SPYRFIKNKANQEYNLRKLLQKVNKIESNGVVAWVGQSENNPGTIATVK |
| | KVIRRNTVLISRMVKEVDGQLFDLTLMKKGKGQVPIKSSDERLTDISKY |
| | GGYNKATGAYFTFVKSKKRGKVVRSFEYVPLHLSKQFENNNELLKEYIE |
| | KDRGLTDVEILIPKVLINSLFRYNGSLVRITGRGDTRLLLVHEQPLYVSNS |
| | FVQQLKSVSSYKLKKSENDNAKLTKTATEKLSNIDELYDGLLRKLDLPIY |
| | SYWFSSIKEYLVESRTKYIKLSIEEKALVIFEILHLFQSDAQVPNLKILGLS |
| | TKPSRIRIQKNLKDTDKMSIIHQSPSGIFEHEIELTSL (SEQ ID NO: 18) |
| AhCas9 | MQNGFLGITVSSEQVGWAVTNPKYELERASRKDLWGVRLFDKAETAED |
| Anaerostipes | RRMFRTNRRLNQRKKNRIHYLRDIFHEEVNQKDPNFFQQLDESNFCEDD |
| hadrus | RTVEFNFDTNLYKNQFPTVYHLRKYLMETKDKPDIRLVYLAFSKFMKN |
| NCBI | RGHFLYKGNLGEVMDFENSMKGFCESLEKFNIDFPTLSDEQVKEVRDIL |
| Reference | CDHKIAKTVKKKNIITITKVKSKTAKAWIGLFCGCSVPVKVLFQDIDEEIV |
| Sequence: | TDPEKISFEDASYDDYIANIEKGVGIYYEAIVSAKMLFDWSILNEILGDHQ |
| WP_044924278.1 | LLSDAMIAEYNKHHDDLKRLQKIIKGTGSRELYQDIFINDVSGNYVCYV |
| Wild type | GHAKTMSSADQKQFYTFLKNRLKNVNGISSEDAEWIDTEIKNGTLLPKQ |
| | TKRDNSVIPHQLQLREFELILDNMQEMYPFLKENREKLLKIFNFVIPYYV |
| | GPLKGVVRKGESTNWMVPKKDGVIHPWNFDEMVDKEASAECFISRMT |
| | GNCSYLFNEKVLPKNSLLYETFEVLNELNPLKINGEPISVELKQRIYEQLF |
| | LTGKKVTKKSLTKYLIKNGYDKDIELSGIDNEFHSNLKSHIDFEDYDNLS |
| | DEEVEQIILRITVFEDKQLLKDYLNREFVKLSEDERKQICSLSYKGWGNL |
| | SEMLLNGITVTDSNGVEVSVMDMLWNTNLNLMQILSKKYGYKAEIEHY |
| | NKEHEKTIYNREDLMDYLNIPPAQRRKVNQLITIVKSLKKTYGVPNKIFF |
| | KISREHQDDPKRTSSRKEQLKYLYKSLKSEDEKHLMKELDELNDHELSN |
| | DKVYLYFLQKGRCIYSGKKLNLSRLRKSNYQNDIDYIYPLSAVNDRSMN |
| | NKVLTGIQENRADKYTYFPVDSEIQKKMKGFWMELVLQGFMTKEKYFR |
| | LSRENDFSKSELVSFIEREISDNQQSGRMIASVLQYYFPESKIVFVKEKLIS |
| | SFKRDFHLISSYGHNHLQAAKDAYITIVVGNVYHTKFTMDPAIYFKNHK |
| | RKDYDLNRLFLENISRDGQIAWESGPYGSIQTVRKEYAQNHIAVTKRVV |
| | EVKGGLFKQMPLKKGHGEYPLKTNDPRFGNIAQYGGYTNVTGSYFVLV |
| | ESMEKGKKRISLEYVPVYLHERLEDDPGHKLLKEYLVDHRKLNHPKILL |
| | AKVRKNSLLKIDGFYYRLNGRSGNALILTNAVELIMDDWQTKTANKISG |
| | YMKRRAIDKKARVYQNEFHIQELEQLYDFYLDKLKNGVYKNRKNNQA |
| | ELIHNEKEQFMELKTEDQCVLLTEIKKLFVCSPMQADLTLIGGSKHTGMI |
| | AMSSNVTKADFAVIAEDPLGLRNKVIYSHKGEK (SEQ ID NO: 19) |
| KvCas9 | MSQNNNKIYNIGLDIGDASVGWAVVDEHYNLLKRHGKHMWGSRLFTQ |
| Kandleria | ANTAVERRSSRSTRRRYNKRRERIRLLREIMEDMVLDVDPTFFIRLANVS |
| vitulina | FLDQEDKKDYLKENYHSNYNLFIDKDFNDKTYYDKYPTIYHLRKHLCES |
| NCBI | KEKEDPRLIYLALHHIVKYRGNFLYEGQKFSMDVSNIEDKMIDVLRQFN |
| Reference | EINLFEYVEDRKKIDEVLNVLKEPLSKKHKAEKAFALFDTTKDNKAAYK |
| Sequence: | ELCAALAGNKFNVTKMLKEAELHDEDEKDISFKFSDATFDDAFVEKQPL |
| WP_031589969.1 | LGDCVEFIDLLHDIYSWVELQNILGSAHTSEPSISAAMIQRYEDHKNDLK |
| Wild type | LLKDVIRKYLPKKYFEVFRDEKSKKNNYCNYINHPSKTPVDEFYKYIKK |
| | LIEKIDDPDVKTILNKIELESFMLKQNSRTNGAVPYQMQLDELNKILENQ |
| | SVYYSDLKDNEDKIRSILTFRIPYYFGPLNITKDRQFDWIIKKEGKENERIL |
| | PWNANEIVDVDKTADEFIKRMRNFCTYFPDEPVMAKNSLTVSKYEVLN |
| | EINKLRINDHLIKRDMKDKMLHTLFMDHKSISANAMKKWLVKNQYFSN |
| | TDDIKIEGFQKENACSTSLTPWIDFTKIFGKINESNYDFIEKIIYDVTVFED |
| | KKILRRRLKKEYDLDEEKIKKILKLKYSGWSRLSKKLLSGIKTKYKDSTR |
| | TPETVLEVMERTNMNLMQVINDEKLGFKKTIDDANSTSVSGKFSYAEVQ |
| | ELAGSPAIKRGIWQALLIVDEIKKIMKHEPAHVYIEFARNEDEKERKDSF |
| | VNQMLKLYKDYDFEDETEKEANKHLKGEDAKSKIRSERLKLYYTQMG |
| | KCMYTGKSLDIDRLDTYQVDHIVPQSLLKDDSIDNKVLVLSSENQRKLD |
| | DLVIPSSIRNKMYGFWEKLFNNKIISPKKFYSLIKTEFNEKDQERFINRQIV |
| | ETRQITKHVAQIIDNHYENTKVVTVRADLSHQFRERYHIYKNRDINDFHH |
| | AHDAYIATILGTYIGHRFESLDAKYIYGEYKRIFRNQKNKGKEMKKNND |
| | GFILNSMRNIYADKDTGEIVWDPNYIDRIKKCFYYKDCFVTKKLEENNG |
| | TFFNVTVLPNDTNSDKDNTLATVPVNKYRSNVNKYGGFSGVNSFIVAIK |
| | GKKKKGKKVIEVNKLTGIPLMYKNADEEIKINYLKQAEDLEEVQIGKEIL |
| | KNQLIEKDGGLYYIVAPTEIINAKQLILNESQTKLVCEIYKAMKYKNYDN |
| | LDSEKIIDLYRLLINKMELYYPEYRKQLVKKFEDRYEQLKVISIEEKCNII |
| | KQILATLHCNSSIGKIMYSDFKISTTIGRLNGRTISLDDISFIAESPTGMYSK |
| | KYKL (SEQ ID NO: 20) |
| EfCas9 | MRLFEEGHTAEDRRLKRTARRRISRRRNRLRYLQAFFEEAMTDLDENFF |
| Enterococcus | ARLQESFLVPEDKKWHRHPIFAKLEDEVAYHETYPTIYHLRKKLADSSE |
| faecalis | QADLRLIYLALAHIVKYRGHFLIEGKLSTENTSVKDQFQQFMVIYNQTFV |
| NCBI | NGESRLVSAPLPESVLIEEELTEKASRTKKSEKVLQQFPQEKANGLFGQF |
| Reference | LKLMVGNKADFKKVFGLEEEAKITYASESYEEDLEGILAKVGDEYSDVF |
| Sequence: | LAAKNVYDAVELSTILADSDKKSHAKLSSSMIVRFTEHQEDLKKFKRFIR |
| WP_016631044.1 | ENCPDEYDNLFKNEQKDGYAGYIAHAGKVSQLKFYQYVKKIIQDIAGAE |
| Wild type | YFLEKIAQENFLRKQRTFDNGVIPHQIHLAELQAIIHRQAAYYPFLKENQE |
| | KIEQLVTFRIPYYVGPLSKGDASTFAWLKRQSEEPIRPWNLQETVDLDQS |
| | ATAFIERMTNFDTYLPSEKVLPKHSLLYEKFMVFNELTKISYTDDRGIKA |
| | NFSGKEKEKIFDYLFKTRRKVKKKDIIQFYRNEYNTEIVTLSGLEEDQFN |
| | ASFSTYQDLLKCGLTRAELDHPDNAEKLEDIIKILTIFEDRQRIRTQLSTFK |
| | GQFSAEVLKKLERKHYTGWGRLSKKLINGIYDKESGKTILDYLVKDDGV |
| | SKHYNRNFMQLINDSQLSFKNAIQKAQSSEHEETLSETVNELAGSPAIKK |
| | GIYQSLKIVDELVAIMGYAPKRIVVEMARENQTTSTGKRRSIQRLKIVEK |
| | AMAEIGSNLLKEQPTTNEQLRDTRLFLYYMQNGKDMYTGDELSLHRLS |
| | HYDIDHIIPQSFMKDDSLDNLVLVGSTENRGKSDDVPSKEVVKDMKAY |
| | WEKLYAAGLISQRKFQRLTKGEQGGLTLEDKAHFIQRQLVETRQITKNV |
| | AGILDQRYNAKSKEKKVQIITLKASLTSQFRSIFGLYKVREVNDYHHGQD |
| | AYLNCVVATTLLKVYPNLAPEFVYGEYPKFQTFKENKATAKAIIYTNLL |
| | RFFTEDEPRFTKDGEILWSNSYLKTIKKELNYHQMNIVKKVEVQKGGFS |
| | KESIKPKGPSNKLIPVKNGLDPQKYGGFDSPVVAYTVLFTHEKGKKPLIK |
| | QEILGITIMEKTRFEQNPILFLEEKGFLRPRVLMKLPKYTLYEFPEGRRRL |
| | LASAKEAQKGNQMVLPEHLLTLLYHAKQCLLPNQSESLAYVEQHQPEF |
| | QEILERVVDFAEVHTLAKSKVQQIVKLFEANQTADVKEIAASFIQLMQFN |
| | AMGAPSTFKFFQKDIERARYTSIKEIFDATIIYQSPTGLYETRRKVVD |
| | (SEQ ID NO: 21) |
| Staphylococcus | KRNYILGLDIGITSVGYGIIDYETRDVIDAGVRLFKEANVENNEGRRSKR |
| aureus | GARRLKRRRRHRIQRVKKLLFDYNLLTDHSELSGINPYEARVKGLSQKL |
| Cas9 | SEEEFSAALLHLAKRRGVHNVNEVEEDTGNELSTKEQISRNSKALEEKY |
| | VAELQLERLKKDGEVRGSINRFKTSDYVKEAKQLLKVQKAYHQLDQSFI |
| | DTYIDLLETRRTYYEGPGEGSPFGWKDIKEWYEMLMGHCTYFPEELRSV |
| | KYAYNADLYNALNDLNNLVITRDENEKLEYYEKFQIIENVFKQKKKPTL |
| | KQIAKEILVNEEDIKGYRVTSTGKPEFTNLKVYHDIKDITARKEIIENAEL |
| | LDQIAKILTIYQSSEDIQEELTNLNSELTQEEIEQISNLKGYTGTHNLSLKAI |
| | NLILDELWHTNDNQIAIFNRLKLVPKKVDLSQQKEIPTTLVDDFILSPVVK |
| | RSFIQSIKVINAIIKKYGLPNDIIIELAREKNSKDAQKMINEMQKRNRQTN |
| | ERIEEIIRTTGKENAKYLIEKIKLHDMQEGKCLYSLEAIPLEDLLNNPFNY |
| | EVDHIIPRSVSFDNSFNNKVLVKQEENSKKGNRTPFQYLSSSDSKISYETF |
| | KKHILNLAKGKGRISKTKKEYLLEERDINRFSVQKDFINRNLVDTRYATR |
| | GLMNLLRSYFRVNNLDVKVKSINGGFTSFLRRKWKFKKERNKGYKHHA |
| | EDALIIANADFIFKEWKKLDKAKKVMENQMFEEKQAESMPEIETEQEYK |
| | EIFITPHQIKHIKDFKDYKYSHRVDKKPNRELINDTLYSTRKDDKGNTLIV |
| | NNLNGLYDKDNDKLKKLINKSPEKLLMYHHDPQTYQKLKLIMEQYGDE |
| | KNPLYKYYEETGNYLTKYSKKDNGPVIKKIKYYGNKLNAHLDITDDYP |
| | NSRNKVVKLSLKPYRFDVYLDNGVYKFVTVKNLDVIKKENYYEVNSKC |
| | YEEAKKLKKISNQAEFIASFYNNDLIKINGELYRVIGVNNDLLNRIEVNMI |
| | DITYREYLENMNDKRPPRIIKTIASKTQSIKKYSTDILGNLYEVKSKKHPQ |
| | IIKKG (SEQ ID NO: 22) |
| Geobacillus | MKYKIGLDIGITSIGWAVINLDIPRIEDLGVRIFDRAENPKTGESLALPRRL |
| thermo- | ARSARRRLRRRKHRLERIRRLFVREGILTKEELNKLFEKKHEIDVWQLRV |
| denitrificans | EALDRKLNNDELARILLHLAKRRGFRSNRKSERTNKENSTMLKHIEENQ |
| Cas9 | SILSSYRTVAEMVVKDPKFSLHKRNKEDNYTNTVARDDLEREIKLIFAKQ |
| | REYGNIVCTEAFEHEYISIWASQRPFASKDDIEKKVGFCTFEPKEKRAPK |
| | ATYTFQSFTVWEHINKLRLVSPGGIRALTDDERRLIYKQAFHKNKITFHD |
| | VRTLLNLPDDTRFKGLLYDRNTTLKENEKVRFLELGAYHKIRKAIDSVY |
| | GKGAAKSFRPIDFDTFGYALTMFKDDTDIRSYLRNEYEQNGKRMENLA |
| | DKVYDEELIEELLNLSFSKFGHLSLKALRNILPYMEQGEVYSTACERAGY |
| | TFTGPKKKQKTVLLPNIPPIANPVVMRALTQARKVVNAIIKKYGSPVSIHI |
| | ELARELSQSFDERRKMQKEQEGNRKKNETAIRQLVEYGLTLNPTGLDIV |
| | KFKLWSEQNGKCAYSLQPIEIERLLEPGYTEVDHVIPYSRSLDDSYTNKV |
| | LVLTKENREKGNRTPAEYLGLGSERWQQFETFVLTNKQFSKKKRDRLLR |
| | LHYDENEENEFKNRNLNDTRYISRFLANFIREHLKFADSDDKQKVYTVN |
| | GRITAHLRSRWNFNKNREESNLHHAVDAAIVACTTPSDIARVTAFYQRR |
| | EQNKELSKKTDPQFPQPWPHFADELQARLSKNPKESIKALNLGNYDNEK |
| | LESLQPVFVSRMPKRSITGAAHQETLRRYIGIDERSGKIQTVVKKKLSEIQ |
| | LDKTGHFPMYGKESDPRTYEAIRQRLLEHNNDPKKAFQEPLYKPKKNGE |
| | LGPIIRTIKIIDTTNQVIPLNDGKTVAYNSNIVRVDVFEKDGKYYCVPIYTI |
| | DMMKGILPNKAIEPNKPYSEWKEMTEDYTFRFSLYPNDLIRIEFPREKTIK |
| | TAVGEEIKIKDLFAYYQTIDSSNGGLSLVSHDNNFSLRSIGSRTLKRFEKY |
| | QVDVLGNIYKVRGEKRVGVASSSHSKAGETIRPL (SEQ ID NO: 23) |
| ScCas9 | MEKKYSIGLDIGTNSVGWAVITDDYKVPSKKFKVLGNTNRKSIKKNLM |
| S. canis | GALLFDSGETAEATRLKRTARRRYTRRKNRIRYLQEIFANEMAKLDDSF |
| | FQRLEESFLVEEDKKNERHPIFGNLADEVAYHRNYPTIYHLRKKLADSPE |
| | KADLRLIYLALAHIIKFRGHFLIEGKLNAENSDVAKLFYQLIQTYNQLFEE |
| | SPLDEIEVDAKGILSARLSKSKRLEKLIAVFPNEKKNGLFGNIIALALGLTP |
| | NFKSNFDLTEDAKLQLSKDTYDDDLDELLGQIGDQYADLFSAAKNLSDA |
| | ILLSDILRSNSEVTKAPLSASMVKRYDEHHQDLALLKTLVRQQFPEKYAE |
| | IFKDDTKNGYAGYVGIGIKHRKRTTKLATQEEFYKFIKPILEKMDGAEEL |
| | LAKLNRDDLLRKQRTFDNGSIPHQIHLKELHAILRRQEEFYPFLKENREKI |
| | EKILTFRIPYYVGPLARGNSRFAWLTRKSEEAITPWNFEEVVDKGASAQS |
| | FIERMTNFDEQLPNKKVLPKHSLLYEYFTVYNELTKVKYVTERMRKPEF |
| | LSGEQKKAIVDLLFKTNRKVTVKQLKEDYFKKIECFDSVEIIGVEDRFNA |
| | SLGTYHDLLKIIKDKDFLDNEENEDILEDIVLTLTLFEDREMIEERLKTYA |
| | HLFDDKVMKQLKRRHYTGWGRLSRKMINGIRDKQSGKTILDFLKSDGF |
| | SNRNFMQLIHDDSLTFKEEIEKAQVSGQGDSLHEQIADLAGSPAIKKGIL |
| | QTVKIVDELVKVMGHKPENIVIEMARENQTTTKGLQQSRERKKRIEEGIK |
| | ELESQILKENPVENTQLQNEKLYLYYLQNGRDMYVDQELDINRLSDYDV |
| | DHIVPQSFIKDDSIDNKVLTRSVENRGKSDNVPSEEVVKKMKNYWRQLL |
| | NAKLITQRKFDNLTKAERGGLSEADKAGFIKRQLVETRQITKHVARILDS |
| | RMNTKRDKNDKPIREVKVITLKSKLVSDFRKDFQLYKVRDINNYHHAH |
| | DAYLNAVVGTALIKKYPKLESEFVYGDYKVYDVRKMIAKSEQEIGKAT |
| | AKRFFYSNIMNFFKTEVKLANGEIRKRPLIETNGETGEVVWNKEKDFAT |
| | VRKVLAMPQVNIVKKTEVQTGGFSKESILSKRESAKLIPRKKGWDTRKY |
| | GGFGSPTVAYSILVVAKVEKGKAKKLKSVKVLVGITIMEKGSYEKDPIGF |
| | LEAKGYKDIKKELIFKLPKYSLFELENGRRRMLASATELQKANELVLPQ |
| | HLVRLLYYTQNISATTGSNNLGYIEQHREEFKEIFEKIIDFSEKYILKNKV |
| | NSNLKSSFDEQFAVSDSILLSNSFVSLLKYTSFGASGGFTFLDLDVKQGRL |
| | RYQTVTEVLDATLIYQSITGLYETRTDLSQLGGD (SEQ ID NO: 24) |

In some embodiments, the napDNAbp used in the prime editors described herein comprise one or more amino acid mutations relative to a wild type napDNAbp, for example, a wild type Cas9 protein. In some embodiments, a Cas9 protein comprises an inactivating mutation in an HNH domain. In some embodiments, the prime editors described herein comprise a Cas9 protein comprising one or more amino acid substitutions relative to the amino acid sequence of SEQ ID NO: 6, or an amino acid sequence at least 80%, at least 85%, at least 90%, at least 95%, at least 96%, at least 97%, at least 98%, or at least 99% identical to SEQ ID NO: 6. In certain embodiments, the Cas9 protein comprises a K775R substitution and/or a K918A substitution relative to the amino acid sequence of SEQ ID NO: 6, or an amino acid sequence at least 80%, at least 85%, at least 90%, at least 95%, at least 96%, at least 97%, at least 98%, or at least 99% identical to SEQ ID NO: 6 (or corresponding mutations in a homologous Cas9 protein). In certain embodiments, the Cas9 protein comprises a K775R substitution and a K918A substitution relative to the amino acid sequence of SEQ ID NO: 6, or an amino acid sequence at least 80%, at least 85%, at least 90%, at least 95%, at least 96%, at least 97%, at least 98%, or at least 99% identical to SEQ ID NO: 6 (or corresponding mutations in a homologous Cas9 protein). In certain embodiments, the Cas9 protein comprises a D23G substitution and/or an H754R substitution relative to the amino acid sequence of SEQ ID NO: 6, or an amino acid sequence at least 80%, at least 85%, at least 90%, at least 95%, at least 96%, at least 97%, at least 98%, or at least 99% identical to SEQ ID NO: 6 (or corresponding mutations in a homologous Cas9 protein). In certain embodiments, the Cas9 protein comprises a D23G substitution and an H754R substitution relative to the amino acid sequence of SEQ ID NO: 6, or an amino acid sequence at least 80%, at least 85%, at least 90%, at least 95%, at least 96%, at least 97%, at least 98%, or at least 99% identical to SEQ ID NO: 6 (or corresponding mutations in a homologous Cas9 protein).

The napDNAbp used in the prime editors described herein may include any suitable homologs and/or orthologs or naturally occurring enzymes, such as Cas9. Cas9 homologs and/or orthologs have been described in various species, including, but not limited to, S. pyogenes and S. thermophilus. The Cas moiety may be configured (e.g., mutagenized, recombinantly engineered, or otherwise obtained from nature) as a nickase, i.e., capable of cleaving only a single strand of the target double-stranded DNA. In some embodiments, a Cas9 nuclease has an inactive (e.g., an inactivated) DNA cleavage domain; that is, the Cas9 is a nickase. In some embodiments, the Cas9 protein comprises an amino acid sequence that is at least 85%, at least 90%, at least 92%, at least 95%, at least 96%, at least 97%, at least 98%, at least 99%, or at least 99.5% identical to the amino acid sequence of a Cas9 protein as provided by any one of the Cas9 orthologs in the above tables.

Additional suitable napDNAbp sequences that can be used in prime editors will be apparent to those of skill in the art based on this disclosure, and such Cas9 nucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737; the entire contents of which are incorporated herein by reference. Additional exemplary Cas variants and homologs include, but are not limited to, Cas9 (e.g., dCas9 and nCas9), Cpf1, CasX, CasY, C2c1, C2c2, C2c3, GeoCas9, CjCas9, Cas12a, Cas12b, Cas12g, Cas12h, Cas12i, Cas13b, Cas13c, Cas13d, Cas14, Csn2, xCas9, SpCas9-NG, Nme2Cas9, circularly permuted Cas9, Argonaute (Ago), Cas9-KKH, SmacCas9, Spy-macCas9, SpCas9-VRQR, SpCas9-NRRH, SpaCas9-NRTH, SpCas9-NRCH, LbCas12a, AsCas12a, CeCas12a, MbCas12a, Cas3, CasΦ, and circularly permuted Cas9 domains such as CP1012, CP1028, CP1041, CP1249, and CP1300, and variants and homologs thereof.

### Reverse Transcriptase Domain

The prime editors used in the complexes and methods described herein comprise a reverse transcriptase domain. In some embodiments, the reverse transcriptase domain is a wild type MMLV reverse transcriptase. In some embodiments, the reverse transcriptase domain is a variant of wild type MMLV reverse transcriptase having the amino acid sequence of SEQ ID NO: 34 (e.g., at least 85%, at least 90%, at least 92%, at least 95%, at least 96%, at least 97%, at least 98%, at least 99%, or at least 99.5% identical to the amino acid sequence of SEQ ID NO: 34).

For example, PE2 and PEmax comprise a variant reverse transcriptase domain of SEQ ID NO: 34, which is based on the wild type MMLV reverse transcriptase domain of SEQ ID NO: 33 (and, in particular, a Genscript codon optimized MMLV reverse transcriptase having the nucleotide sequence of SEQ ID NO: 33), and which comprises amino acid substitutions D200N, T306K, W313F, T330P, and L603W relative to the wild type MMLV RT of SEQ ID NO: 33. The amino acid sequence of the variant RT domain of PE2 and PEmax is SEQ ID NO: 34.

Prime editors may also comprise other variant RT domains as well. In various embodiments, the prime editors used in the methods and systems described herein (with the RT domain provided as either a fusion partner or in trans) can include a variant RT comprising one or more of the following mutations: P51L, S67K, E69K, L139P, T197A, D200N, H204R, F209N, E302K, E302R, T306K, F309N, W313F, T330P, L345G, L435G, N454K, D524G, E562Q, D583N, H594Q, L603W, E607K, or D653N in the wild type M-MLV RT of SEQ ID NO: 33, or at a corresponding amino acid position in another wild type RT polypeptide sequence.

Some exemplary reverse transcriptases that can be fused to napDNAbp proteins or provided as individual proteins according to various embodiments of this disclosure are provided below. Exemplary reverse transcriptases include variants with at least 80%, at least 85%, at least 90%, at least 95%, at least 98%, or at least 99% sequence identity to the following wild-type enzymes or partial enzymes:

| Description | Sequence (variant substitutions relative to wild type) |
|---|---|
| Reverse | TLNIEDEYRLHETSKEPDVSLGSTWLSDFPQAWAETGGMGLAVRQAP |
| transcriptase | LIIPLKATSTPVSIKQYPMSQEARLGIKPHIQRLLDQGILVPCQSPWNTPL |
| (M-MLV RT) | LPVKKPGTNDYRPVQDLREVNKRVEDIHPTVPNPYNLLSGLPPSHQWY |
| wild type | TVLDLKDAFFCLRLHPTSQPLFAFEWRDPEMGISGQLTWTRLPQGFKN |
| Moloney | SPTLFDEALHRDLADFRIQHPDLILLQYVDDLLLAATSELDCQQGTRAL |
| murine | LQTLGNLGYRASAKKAQICQKQVKYLGYLLKEGQRWLTEARKETVM |
| leukemia | GQPTPKTPRQLREFLGTAGFCRLWIPGFAEMAAPLYPLTKTGTLFNWG |
| virus | PDQQKAYQEIKQALLTAPALGLPDLTKPFELFVDEKQGYAKGVLTQKL |
| Used in PE1 | GPWRRPVAYLSKKLDPVAAGWPPCLRMVAAIAVLTKDAGKLTMGQP |
| (prime editor | LVILAPHAVEALVKQPPDRWLSNARMTHYQALLLDTDRVQFGPVVAL |
| 1 fusion | NPATLLPLPEEGLQHNCLDILAEAHGTRPDLTDQPLPDADHTWYTDGS |
| protein | SLLQEGQRKAGAAVTTETEVIWAKALPAGTSAQRAELIALTQALKMA |
| disclosed | EGKKLNVYTDSRYAFATAHIHGEIYRRRGLLTSEGKEIKNKDEILALLK |
| herein) | ALFLPKRLSIIHCPGHQKGHSAEARGNRMADQAARKAAITETPDTSTLL |
| | IENSSP (SEQ ID NO: 33) |
| M-MLV RT | TLNIEDEYRLHETSKEPDVSLGSTWLSDFPQAWAETGGMGLAVRQAP |
| D200N | LIIPLKATSTPVSIKQYPMSQEARLGIKPHIQRLLDQGILVPCQSPWNTPL |
| | LPVKKPGTNDYRPVQDLREVNKRVEDIHPTVPNPYNLLSGLPPSHQWY |
| | TVLDLKDAFFCLRLHPTSQPLFAFEWRDPEMGISGQLTWTRLPQGFKN |
| | SPTLFNEALHRDLADFRIQHPDLILLQYVDDLLLAATSELDCQQGTRAL |
| | LQTLGNLGYRASAKKAQICQKQVKYLGYLLKEGQRWLTEARKETVM |
| | GQPTPKTPRQLREFLGTAGFCRLWIPGFAEMAAPLYPLTKTGTLFNWG |
| | PDQQKAYQEIKQALLTAPALGLPDLTKPFELFVDEKQGYAKGVLTQKL |
| | GPWRRPVAYLSKKLDPVAAGWPPCLRMVAAIAVLTKDAGKLTMGQP |
| | LVILAPHAVEALVKQPPDRWLSNARMTHYQALLLDTDRVQFGPVVAL |
| | NPATLLPLPEEGLQHNCLDILAEAHGTRPDLTDQPLPDADHTWYTDGS |
| | SLLQEGQRKAGAAVTTETEVIWAKALPAGTSAQRAELIALTQALKMA |
| | EGKKLNVYTDSRYAFATAHIHGEIYRRRGLLTSEGKEIKNKDEILALLK |
| | ALFLPKRLSIIHCPGHQKGHSAEARGNRMADQAARKAAITETPDTSTLL |
| | IENSSP (SEQ ID NO: 63) |
| M-MLV RT | TLNIEDEYRLHETSKEPDVSLGSTWLSDFPQAWAETGGMGLAVRQAP |
| D200N | LIIPLKATSTPVSIKQYPMSQEARLGIKPHIQRLLDQGILVPCQSPWNTPL |
| T330P | LPVKKPGTNDYRPVQDLREVNKRVEDIHPTVPNPYNLLSGLPPSHQWY |
| | TVLDLKDAFFCLRLHPTSQPLFAFEWRDPEMGISGQLTWTRLPQGFKN |
| | SPTLFNEALHRDLADFRIQHPDLILLQYVDDLLLAATSELDCQQGTRAL |
| | LQTLGNLGYRASAKKAQICQKQVKYLGYLLKEGQRWLTEARKETVM |
| | GQPTPKTPRQLREFLGTAGFCRLWIPGFAEMAAPLYPLTKPGTLFNWG |
| | PDQQKAYQEIKQALLTAPALGLPDLTKPFELFVDEKQGYAKGVLTQKL |
| | GPWRRPVAYLSKKLDPVAAGWPPCLRMVAAIAVLTKDAGKLTMGQP |
| | LVILAPHAVEALVKQPPDRWLSNARMTHYQALLLDTDRVQFGPVVAL |
| | NPATLLPLPEEGLQHNCLDILAEAHGTRPDLTDQPLPDADHTWYTDGS |
| | SLLQEGQRKAGAAVTTETEVIWAKALPAGTSAQRAELIALTQALKMA |
| | EGKKLNVYTDSRYAFATAHIHGEIYRRRGLLTSEGKEIKNKDEILALLK |
| | ALFLPKRLSIIHCPGHQKGHSAEARGNRMADQAARKAAITETPDTSTLL |
| | IENSSP (SEQ ID NO: 64) |
| M-MLV RT | TLNIEDEYRLHETSKEPDVSLGSTWLSDFPQAWAETGGMGLAVRQAP |
| D200N | LIIPLKATSTPVSIKQYPMSQEARLGIKPHIQRLLDQGILVPCQSPWNTPL |
| T330P | LPVKKPGTNDYRPVQDLREVNKRVEDIHPTVPNPYNLLSGLPPSHQWY |
| L603W | TVLDLKDAFFCLRLHPTSQPLFAFEWRDPEMGISGQLTWTRLPQGFKN |
| | SPTLFNEALHRDLADFRIQHPDLILLQYVDDLLLAATSELDCQQGTRAL |
| | LQTLGNLGYRASAKKAQICQKQVKYLGYLLKEGQRWLTEARKETVM |
| | GQPTPKTPRQLREFLGTAGFCRLWIPGFAEMAAPLYPLTKPGTLFNWG |
| | PDQQKAYQEIKQALLTAPALGLPDLTKPFELFVDEKQGYAKGVLTQKL |
| | GPWRRPVAYLSKKLDPVAAGWPPCLRMVAAIAVLTKDAGKLTMGQP |
| | LVILAPHAVEALVKQPPDRWLSNARMTHYQALLLDTDRVQFGPVVAL |
| | NPATLLPLPEEGLQHNCLDILAEAHGTRPDLTDQPLPDADHTWYTDGS |
| | SLLQEGQRKAGAAVTTETEVIWAKALPAGTSAQRAELIALTQALKMA |
| | EGKKLNVYTDSRYAFATAHIHGEIYRRRGWLTSEGKEIKNKDEILALL |
| | KALFLPKRLSIIHCPGHQKGHSAEARGNRMADQAARKAAITETPDTST |
| | LLIENSSP (SEQ ID NO: 65) |
| M-MLV RT | TLNIEDEYRLHETSKEPDVSLGSTWLSDFPQAWAETGGMGLAVRQAP |
| D200N | LIIPLKATSTPVSIKQYPMSQKARLGIKPHIQRLLDQGILVPCQSPWNTP |
| T330P | LLPVKKPGTNDYRPVQDLREVNKRVEDIHPTVPNPYNLLSGLPPSHQW |
| L603W | YTVLDLKDAFFCLRLHPTSQPLFAFEWRDPEMGISGQLTWTRLPQGFK |
| E69K | NSPTLFNEALHRDLADFRIQHPDLILLQYVDDLLLAATSELDCQQGTRA |
| | LLQTLGNLGYRASAKKAQICQKQVKYLGYLLKEGQRWLTEARKETV |
| | MGQPTPKTPRQLREFLGTAGFCRLWIPGFAEMAAPLYPLTKPGTLFNW |
| | GPDQQKAYQEIKQALLTAPALGLPDLTKPFELFVDEKQGYAKGVLTQ |
| | KLGPWRRPVAYLSKKLDPVAAGWPPCLRMVAAIAVLTKDAGKLTMG |
| | QPLVILAPHAVEALVKQPPDRWLSNARMTHYQALLLDTDRVQFGPVV |
| | ALNPATLLPLPEEGLQHNCLDILAEAHGTRPDLTDQPLPDADHTWYTD |
| | GSSLLQEGQRKAGAAVTTETEVIWAKALPAGTSAQRAELIALTQALK |
| | MAEGKKLNVYTDSRYAFATAHIHGEIYRRRGWLTSEGKEIKNKDEILA |
| | LLKALFLPKRLSIIHCPGHQKGHSAEARGNRMADQAARKAAITETPDT |
| | STLLIENSSP (SEQ ID NO: 66) |
| M-MLV RT | TLNIEDEYRLHETSKEPDVSLGSTWLSDFPQAWAETGGMGLAVRQAP |
| D200N | LIIPLKATSTPVSIKQYPMSQEARLGIKPHIQRLLDQGILVPCQSPWNTPL |
| T330P | LPVKKPGTNDYRPVQDLREVNKRVEDIHPTVPNPYNLLSGLPPSHQWY |
| L603W | TVLDLKDAFFCLRLHPTSQPLFAFEWRDPEMGISGQLTWTRLPQGFKN |
| E302R | SPTLFNEALHRDLADFRIQHPDLILLQYVDDLLLAATSELDCQQGTRAL |
| | LQTLGNLGYRASAKKAQICQKQVKYLGYLLKEGQRWLTEARKETVM |
| | GQPTPKTPRQLRRFLGTAGFCRLWIPGFAEMAAPLYPLTKPGTLFNWG |
| | PDQQKAYQEIKQALLTAPALGLPDLTKPFELFVDEKQGYAKGVLTQKL |
| | GPWRRPVAYLSKKLDPVAAGWPPCLRMVAAIAVLTKDAGKLTMGQP |
| | LVILAPHAVEALVKQPPDRWLSNARMTHYQALLLDTDRVQFGPVVAL |
| | NPATLLPLPEEGLQHNCLDILAEAHGTRPDLTDQPLPDADHTWYTDGS |
| | SLLQEGQRKAGAAVTTETEVIWAKALPAGTSAQRAELIALTQALKMA |
| | EGKKLNVYTDSRYAFATAHIHGEIYRRRGWLTSEGKEIKNKDEILALL |
| | KALFLPKRLSIIHCPGHQKGHSAEARGNRMADQAARKAAITETPDTST |
| | LLIENSSP(SEQ ID NO: 67) |
| M-MLV RT | TLNIEDEYRLHETSKEPDVSLGSTWLSDFPQAWAETGGMGLAVRQAP |
| D200N | LIIPLKATSTPVSIKQYPMSQEARLGIKPHIQRLLDQGILVPCQSPWNTPL |
| T330P | LPVKKPGTNDYRPVQDLREVNKRVEDIHPTVPNPYNLLSGLPPSHQWY |
| L603W | TVLDLKDAFFCLRLHPTSQPLFAFEWRDPEMGISGQLTWTRLPQGFKN |
| E607K | SPTLFNEALHRDLADFRIQHPDLILLQYVDDLLLAATSELDCQQGTRAL |
| | LQTLGNLGYRASAKKAQICQKQVKYLGYLLKEGQRWLTEARKETVM |
| | GQPTPKTPRQLREFLGTAGFCRLWIPGFAEMAAPLYPLTKPGTLFNWG |
| | PDQQKAYQEIKQALLTAPALGLPDLTKPFELFVDEKQGYAKGVLTQKL |
| | GPWRRPVAYLSKKLDPVAAGWPPCLRMVAAIAVLTKDAGKLTMGQP |
| | LVILAPHAVEALVKQPPDRWLSNARMTHYQALLLDTDRVQFGPVVAL |
| | NPATLLPLPEEGLQHNCLDILAEAHGTRPDLTDQPLPDADHTWYTDGS |
| | SLLQEGQRKAGAAVTTETEVIWAKALPAGTSAQRAELIALTQALKMA |
| | EGKKLNVYTDSRYAFATAHIHGEIYRRRGWLTSKGKEIKNKDEILALL |
| | KALFLPKRLSIIHCPGHQKGHSAEARGNRMADQAARKAAITETPDTST |
| | LLIENSSP (SEQ ID NO: 68) |
| M-MLV RT | TLNIEDEYRLHETSKEPDVSLGSTWLSDFPQAWAETGGMGLAVRQAP |
| D200N | LIIPLKATSTPVSIKQYPMSQEARLGIKPHIQRLLDQGILVPCQSPWNTPL |
| T330P | LPVKKPGTNDYRPVQDLREVNKRVEDIHPTVPNPYNLLSGPPPSHQWY |
| L603W | TVLDLKDAFFCLRLHPTSQPLFAFEWRDPEMGISGQLTWTRLPQGFKN |
| L139P | SPTLFNEALHRDLADFRIQHPDLILLQYVDDLLLAATSELDCQQGTRAL |
| | LQTLGNLGYRASAKKAQICQKQVKYLGYLLKEGQRWLTEARKETVM |
| | GQPTPKTPRQLREFLGTAGFCRLWIPGFAEMAAPLYPLTKPGTLFNWG |
| | PDQQKAYQEIKQALLTAPALGLPDLTKPFELFVDEKQGYAKGVLTQKL |
| | GPWRRPVAYLSKKLDPVAAGWPPCLRMVAAIAVLTKDAGKLTMGQP |
| | LVILAPHAVEALVKQPPDRWLSNARMTHYQALLLDTDRVQFGPVVAL |
| | NPATLLPLPEEGLQHNCLDILAEAHGTRPDLTDQPLPDADHTWYTDGS |
| | SLLQEGQRKAGAAVTTETEVIWAKALPAGTSAQRAELIALTQALKMA |
| | EGKKLNVYTDSRYAFATAHIHGEIYRRRGWLTSEGKEIKNKDEILALL |
| | KALFLPKRLSIIHCPGHQKGHSAEARGNRMADQAARKAAITETPDTST |
| | LLIENSSP (SEQ ID NO: 69) |
| M-MLV RT | TLNIEDEYRLHETSKEPDVSLGSTWLSDFPQAWAETGGMGLAVRQAP |
| D200N | LIIPLKATSTPVSIKQYPMSQEARLGIKPHIQRLLDQGILVPCQSPWNTPL |
| T330P | LPVKKPGTNDYRPVQDLREVNKRVEDIHPTVPNPYNLLSGLPPSHQWY |
| L603W | TVLDLKDAFFCLRLHPTSQPLFAFEWRDPEMGISGQLTWTRLPQGFKN |
| L435G | SPTLFNEALHRDLADFRIQHPDLILLQYVDDLLLAATSELDCQQGTRAL |
| | LQTLGNLGYRASAKKAQICQKQVKYLGYLLKEGQRWLTEARKETVM |
| | GQPTPKTPRQLREFLGTAGFCRLWIPGFAEMAAPLYPLTKPGTLFNWG |
| | PDQQKAYQEIKQALLTAPALGLPDLTKPFELFVDEKQGYAKGVLTQKL |
| | GPWRRPVAYLSKKLDPVAAGWPPCLRMVAAIAVLTKDAGKLTMGQP |
| | LVIGAPHAVEALVKQPPDRWLSNARMTHYQALLLDTDRVQFGPVVAL |
| | NPATLLPLPEEGLQHNCLDILAEAHGTRPDLTDQPLPDADHTWYTDGS |
| | SLLQEGQRKAGAAVTTETEVIWAKALPAGTSAQRAELIALTQALKMA |
| | EGKKLNVYTDSRYAFATAHIHGEIYRRRGWLTSEGKEIKNKDEILALL |
| | KALFLPKRLSIIHCPGHQKGHSAEARGNRMADQAARKAAITETPDTST |
| | LLIENSSP (SEQ ID NO: 70) |
| M-MLV RT | TLNIEDEYRLHETSKEPDVSLGSTWLSDFPQAWAETGGMGLAVRQAP |
| D200N | LIIPLKATSTPVSIKQYPMSQEARLGIKPHIQRLLDQGILVPCQSPWNTPL |
| T330P | LPVKKPGTNDYRPVQDLREVNKRVEDIHPTVPNPYNLLSGLPPSHQWY |
| L603W | TVLDLKDAFFCLRLHPTSQPLFAFEWRDPEMGISGQLTWTRLPQGFKN |
| N454K | SPTLFNEALHRDLADFRIQHPDLILLQYVDDLLLAATSELDCQQGTRAL |
| | LQTLGNLGYRASAKKAQICQKQVKYLGYLLKEGQRWLTEARKETVM |
| | GQPTPKTPRQLREFLGTAGFCRLWIPGFAEMAAPLYPLTKPGTLFNWG |
| | PDQQKAYQEIKQALLTAPALGLPDLTKPFELFVDEKQGYAKGVLTQKL |
| | GPWRRPVAYLSKKLDPVAAGWPPCLRMVAAIAVLTKDAGKLTMGQP |
| | LVILAPHAVEALVKQPPDRWLSKARMTHYQALLLDTDRVQFGPVVAL |
| | NPATLLPLPEEGLQHNCLDILAEAHGTRPDLTDQPLPDADHTWYTDGS |
| | SLLQEGQRKAGAAVTTETEVIWAKALPAGTSAQRAELIALTQALKMA |
| | EGKKLNVYTDSRYAFATAHIHGEIYRRRGWLTSEGKEIKNKDEILALL |
| | KALFLPKRLSIIHCPGHQKGHSAEARGNRMADQAARKAAITETPDTST |
| | LLIENSSP (SEQ ID NO: 71) |
| M-MLV RT | TLNIEDEYRLHETSKEPDVSLGSTWLSDFPQAWAETGGMGLAVRQAP |
| D200N | LIIPLKATSTPVSIKQYPMSQEARLGIKPHIQRLLDQGILVPCQSPWNTPL |
| T330P | LPVKKPGTNDYRPVQDLREVNKRVEDIHPTVPNPYNLLSGLPPSHQWY |
| L603W | TVLDLKDAFFCLRLHPTSQPLFAFEWRDPEMGISGQLTWTRLPQGFKN |
| T306K | SPTLFNEALHRDLADFRIQHPDLILLQYVDDLLLAATSELDCQQGTRAL |
| | LQTLGNLGYRASAKKAQICQKQVKYLGYLLKEGQRWLTEARKETVM |
| | GQPTPKTPRQLREFLGKAGFCRLWIPGFAEMAAPLYPLTKPGTLFNWG |
| | PDQQKAYQEIKQALLTAPALGLPDLTKPFELFVDEKQGYAKGVLTQKL |
| | GPWRRPVAYLSKKLDPVAAGWPPCLRMVAAIAVLTKDAGKLTMGQP |
| | LVILAPHAVEALVKQPPDRWLSNARMTHYQALLLDTDRVQFGPVVAL |
| | NPATLLPLPEEGLQHNCLDILAEAHGTRPDLTDQPLPDADHTWYTDGS |
| | SLLQEGQRKAGAAVTTETEVIWAKALPAGTSAQRAELIALTQALKMA |
| | EGKKLNVYTDSRYAFATAHIHGEIYRRRGWLTSEGKEIKNKDEILALL |
| | KALFLPKRLSIIHCPGHQKGHSAEARGNRMADQAARKAAITETPDTST |
| | LLIENSSP (SEQ ID NO: 72) |
| M-MLV RT | TLNIEDEYRLHETSKEPDVSLGSTWLSDFPQAWAETGGMGLAVRQAP |
| D200N | LIIPLKATSTPVSIKQYPMSQEARLGIKPHIQRLLDQGILVPCQSPWNTPL |
| T330P | LPVKKPGTNDYRPVQDLREVNKRVEDIHPTVPNPYNLLSGLPPSHQWY |
| L603W | TVLDLKDAFFCLRLHPTSQPLFAFEWRDPEMGISGQLTWTRLPQGFKN |
| W313F | SPTLFNEALHRDLADFRIQHPDLILLQYVDDLLLAATSELDCQQGTRAL |
| | LQTLGNLGYRASAKKAQICQKQVKYLGYLLKEGQRWLTEARKETVM |
| | GQPTPKTPRQLREFLGTAGFCRLFIPGFAEMAAPLYPLTKPGTLFNWGP |
| | DQQKAYQEIKQALLTAPALGLPDLTKPFELFVDEKQGYAKGVLTQKL |
| | GPWRRPVAYLSKKLDPVAAGWPPCLRMVAAIAVLTKDAGKLTMGQP |
| | LVILAPHAVEALVKQPPDRWLSNARMTHYQALLLDTDRVQFGPVVAL |
| | NPATLLPLPEEGLQHNCLDILAEAHGTRPDLTDQPLPDADHTWYTDGS |
| | SLLQEGQRKAGAAVTTETEVIWAKALPAGTSAQRAELIALTQALKMA |
| | EGKKLNVYTDSRYAFATAHIHGEIYRRRGWLTSEGKEIKNKDEILALL |
| | KALFLPKRLSIIHCPGHQKGHSAEARGNRMADQAARKAAITETPDTST |
| | LLIENSSP (SEQ ID NO: 73) |
| M-MLV RT | TLNIEDEYRLHETSKEPDVSLGSTWLSDFPQAWAETGGMGLAVRQAP |
| D200N | LIIPLKATSTPVSIKQYPMSQEARLGIKPHIQRLLDQGILVPCQSPWNTPL |
| T330P | LPVKKPGTNDYRPVQDLREVNKRVEDIHPTVPNPYNLLSGLPPSHQWY |
| L603W | TVLDLKDAFFCLRLHPTSQPLFAFEWRDPEMGISGQLTWTRLPQGFKN |
| D524G | SPTLFNEALHRDLADFRIQHPDLILLQYVDDLLLAATSELDCQQGTRAL |
| E562Q | LQTLGNLGYRASAKKAQICQKQVKYLGYLLKEGQRWLTEARKETVM |
| D583N | GQPTPKTPRQLREFLGTAGFCRLWIPGFAEMAAPLYPLTKPGTLFNWG |
| | PDQQKAYQEIKQALLTAPALGLPDLTKPFELFVDEKQGYAKGVLTQKL |
| | GPWRRPVAYLSKKLDPVAAGWPPCLRMVAAIAVLTKDAGKLTMGQP |
| | LVILAPHAVEALVKQPPDRWLSNARMTHYQALLLDTDRVQFGPVVAL |
| | NPATLLPLPEEGLQHNCLDILAEAHGTRPDLTDQPLPDADHTWYTGGS |
| | SLLQEGQRKAGAAVTTETEVIWAKALPAGTSAQRAQLIALTQALKMA |
| | EGKKLNVYTNSRYAFATAHIHGEIYRRRGWLTSEGKEIKNKDEILALL |
| | KALFLPKRLSIIHCPGHQKGHSAEARGNRMADQAARKAAITETPDTST |
| | LLIENSSP (SEQ ID NO: 74) |
| M-MLV RT | TLNIEDEYRLHETSKEPDVSLGSTWLSDFPQAWAETGGMGLAVRQAP |
| D200N | LIIPLKATSTPVSIKQYPMSQEARLGIKPHIQRLLDQGILVPCQSPWNTPL |
| T330P | LPVKKPGTNDYRPVQDLREVNKRVEDIHPTVPNPYNLLSGLPPSHQWY |
| L603W | TVLDLKDAFFCLRLHPTSQPLFAFEWRDPEMGISGQLTWTRLPQGFKN |
| E302R | SPTLFNEALHRDLADFRIQHPDLILLQYVDDLLLAATSELDCQQGTRAL |
| W313F | LQTLGNLGYRASAKKAQICQKQVKYLGYLLKEGQRWLTEARKETVM |
| | GQPTPKTPRQLRRFLGTAGFCRLFIPGFAEMAAPLYPLTKPGTLFNWG |
| | PDQQKAYQEIKQALLTAPALGLPDLTKPFELFVDEKQGYAKGVLTQKL |
| | GPWRRPVAYLSKKLDPVAAGWPPCLRMVAAIAVLTKDAGKLTMGQP |
| | LVILAPHAVEALVKQPPDRWLSNARMTHYQALLLDTDRVQFGPVVAL |
| | NPATLLPLPEEGLQHNCLDILAEAHGTRPDLTDQPLPDADHTWYTDGS |
| | SLLQEGQRKAGAAVTTETEVIWAKALPAGTSAQRAELIALTQALKMA |
| | EGKKLNVYTDSRYAFATAHIHGEIYRRRGWLTSEGKEIKNKDEILALL |
| | KALFLPKRLSIIHCPGHQKGHSAEARGNRMADQAARKAAITETPDTST |
| | LLIENSSP (SEQ ID NO: 75) |
| M-MLV RT | TLNIEDEYRLHETSKEPDVSLGSTWLSDFPQAWAETGGMGLAVRQAP |
| D200N | LIIPLKATSTPVSIKQYPMSQEARLGIKPHIQRLLDQGILVPCQSPWNTPL |
| T330P | LPVKKPGTNDYRPVQDLREVNKRVEDIHPTVPNPYNLLSGPPPSHQWY |
| L603W | TVLDLKDAFFCLRLHPTSQPLFAFEWRDPEMGISGQLTWTRLPQGFKN |
| E607K | SPTLFNEALHRDLADFRIQHPDLILLQYVDDLLLAATSELDCQQGTRAL |
| L139P | LQTLGNLGYRASAKKAQICQKQVKYLGYLLKEGQRWLTEARKETVM |
| | GQPTPKTPRQLREFLGTAGFCRLWIPGFAEMAAPLYPLTKPGTLFNWG |
| | PDQQKAYQEIKQALLTAPALGLPDLTKPFELFVDEKQGYAKGVLTQKL |
| | GPWRRPVAYLSKKLDPVAAGWPPCLRMVAAIAVLTKDAGKLTMGQP |
| | LVILAPHAVEALVKQPPDRWLSNARMTHYQALLLDTDRVQFGPVVAL |
| | NPATLLPLPEEGLQHNCLDILAEAHGTRPDLTDQPLPDADHTWYTDGS |
| | SLLQEGQRKAGAAVTTETEVIWAKALPAGTSAQRAELIALTQALKMA |
| | EGKKLNVYTDSRYAFATAHIHGEIYRRRGWLTSKGKEIKNKDEILALL |
| | KALFLPKRLSIIHCPGHQKGHSAEARGNRMADQAARKAAITETPDTST |
| | LLIENSSP (SEQ ID NO: 76) |
| M-MLV RT | TLNIEDEYRLHETSKEPDVSLGSTWLSDFPQAWAETGGMGLAVRQAP |
| P51L | LIILLKATSTPVSIKQYPMKQEARLGIKPHIQRLLDQGILVPCQSPWNTP |
| S67K | LLPVKKPGTNDYRPVQDLREVNKRVEDIHPTVPNPYNLLSGLPPSHQW |
| T197A | YTVLDLKDAFFCLRLHPTSQPLFAFEWRDPEMGISGQLTWTRLPQGFK |
| H204R | NSPALFDEALRRDLADFRIQHPDLILLQYVDDLLLAATSELDCQQGTR |
| E302K | ALLQTLGNLGYRASAKKAQICQKQVKYLGYLLKEGQRWLTEARKET |
| F309N | VMGQPTPKTPRQLRKFLGTAGNCRLFIPGFAEMAAPLYPLTKPGTLFN |
| W313F | WGPDQQKAYQEIKQALLTAPALGLPDLTKPFELFVDEKQGYAKGVLT |
| T330P | QKLGPWRRPVAYLSKKLDPVAAGWPPCLRMVAAIAVLTKDAGKLTM |
| L435G | GQPLVIGAPHAVEALVKQPPDRWLSKARMTHYQALLLDTDRVQFGPV |
| N454K | VALNPATLLPLPEEGLQHNCLDILAEAHGTRPDLTDQPLPDADHTWYT |
| D524G | GGSSLLQEGQRKAGAAVTTETEVIWAKALPAGTSAQRAELIALTQALK |
| D583N | MAEGKKLNVYTNSRYAFATAHIQGEIYRRRGLLTSEGKEIKNKDEILA |
| H594Q | LLKALFLPKRLSIIHCPGHQKGHSAEARGNRMANQAARKAAITETPDT |
| D653N | STLLIENSSP (SEQ ID NO: 77) |
| M-MLV RT | TLNIEDEYRLHETSKEPDVSLGSTWLSDFPQAWAETGGMGLAVRQAP |
| D200N | LIILLKATSTPVSIKQYPMKQEARLGIKPHIQRLLDQGILVPCQSPWNTP |
| P51L | LLPVKKPGTNDYRPVQDLREVNKRVEDIHPTVPNPYNLLSGLPPSHQW |
| S67K | YTVLDLKDAFFCLRLHPTSQPLFAFEWRDPEMGISGQLTWTRLPQGFK |
| T197A | NSPALFNEALRRDLADFRIQHPDLILLQYVDDLLLAATSELDCQQGTR |
| H204R | ALLQTLGNLGYRASAKKAQICQKQVKYLGYLLKEGQRWLTEARKET |
| E302K | VMGQPTPKTPRQLRKFLGTAGNCRLFIPGFAEMAAPLYPLTKPGTLFN |
| F309N | WGPDQQKAYQEIKQALLTAPALGLPDLTKPFELFVDEKQGYAKGVLT |
| W313F | QKLGPWRRPVAYLSKKLDPVAAGWPPCLRMVAAIAVLTKDAGKLTM |
| T330P | GQPLVIGAPHAVEALVKQPPDRWLSKARMTHYQALLLDTDRVQFGPV |
| L345G | VALNPATLLPLPEEGLQHNCLDILAEAHGTRPDLTDQPLPDADHTWYT |
| N454K | GGSSLLQEGQRKAGAAVTTETEVIWAKALPAGTSAQRAELIALTQALK |
| D524G | MAEGKKLNVYTNSRYAFATAHIQGEIYRRRGLLTSEGKEIKNKDEILA |
| D583N | LLKALFLPKRLSIIHCPGHQKGHSAEARGNRMANQAARKAAITETPDT |
| H594Q | STLLIENSSP (SEQ ID NO: 78) |
| D653N | |
| M-MLV RT | TLNIEDEYRLHETSKEPDVSLGSTWLSDFPQAWAETGGMGLAVRQAP |
| D200N | LIIPLKATSTPVSIKQYPMSQEARLGIKPHIQRLLDQGILVPCQSPWNTPL |
| T330P | LPVKKPGTNDYRPVQDLREVNKRVEDIHPTVPNPYNLLSGLPPSHQWY |
| L603W | TVLDLKDAFFCLRLHPTSQPLFAFEWRDPEMGISGQLTWTRLPQGFKN |
| T306K | SPTLFNEALHRDLADFRIQHPDLILLQYVDDLLLAATSELDCQQGTRAL |
| W313F | LQTLGNLGYRASAKKAQICQKQVKYLGYLLKEGQRWLTEARKETVM |
| | GQPTPKTPRQLREFLGKAGFCRLFIPGFAEMAAPLYPLTKPGTLFNWG |
| | PDQQKAYQEIKQALLTAPALGLPDLTKPFELFVDEKQGYAKGVLTQKL |
| | GPWRRPVAYLSKKLDPVAAGWPPCLRMVAAIAVLTKDAGKLTMGQP |
| | LVILAPHAVEALVKQPPDRWLSNARMTHYQALLLDTDRVQFGPVVAL |
| | NPATLLPLPEEGLQHNCLDILAEAHGTRPDLTDQPLPDADHTWYTDGS |
| | SLLQEGQRKAGAAVTTETEVIWAKALPAGTSAQRAELIALTQALKMA |
| | EGKKLNVYTDSRYAFATAHIHGEIYRRRGWLTSEGKEIKNKDEILALL |
| | KALFLPKRLSIIHCPGHQKGHSAEARGNRMADQAARKAAITETPDTST |
| | LLIENSSP (SEQ ID NO: 34) |

In various embodiments, the prime editors utilized in the complexes and methods described herein (with RT provided as either a fusion partner or in trans) can include a variant RT comprising one or more of the following mutations: P51X, S67X, E69X, L139X, T197X, D200X, H204X, F209X, E302X, T306X, F309X, W313X, T330X, L345X, L435X, N454X, D524X, E562X, D583X, H594X, L603X, E607X, or D653X in the wild type M-MLV RT of SEQ ID NO: 33, or at a corresponding amino acid position in another wild type RT polypeptide sequence, wherein “X” can be any amino acid.

In various embodiments, the prime editors used in the complexes and methods described herein (with RT provided as either a fusion partner or in trans) can include a variant RT comprising a P51X mutation in the wild type M-MLV RT of SEQ ID NO: 33, or at a corresponding amino acid position in another wild type RT polypeptide sequence, wherein “X” can be any amino acid. In certain embodiments, X is L.

In various embodiments, the prime editors used in the complexes and methods described herein (with RT provided as either a fusion partner or in trans) can include a variant RT comprising an S67X mutation in the wild type M-MLV RT of SEQ ID NO: 33, or at a corresponding amino acid position in another wild type RT polypeptide sequence, wherein “X” can be any amino acid. In certain embodiments, X is K.

In various embodiments, the prime editors used in the complexes and methods described herein (with RT provided as either a fusion partner or in trans) can include a variant RT comprising an E69X mutation in the wild type M-MLV RT of SEQ ID NO: 33, or at a corresponding amino acid position in another wild type RT polypeptide sequence, wherein “X” can be any amino acid. In certain embodiments, X is K.

In various embodiments, the prime editors used in the complexes and methods described herein (with RT provided as either a fusion partner or in trans) can include a variant RT comprising an L139X mutation in the wild type M-MLV RT of SEQ ID NO: 33, or at a corresponding amino acid position in another wild type RT polypeptide sequence, wherein “X” can be any amino acid. In certain embodiments, X is P.

In various embodiments, the prime editors used in the complexes and methods described herein (with RT provided as either a fusion partner or in trans) can include a variant RT comprising a T197X mutation in the wild type M-MLV RT of SEQ ID NO: 33, or at a corresponding amino acid position in another wild type RT polypeptide sequence, wherein “X” can be any amino acid. In certain embodiments, X is A.

In various embodiments, the prime editors used in the complexes and methods described herein (with RT provided as either a fusion partner or in trans) can include a variant RT comprising a D200X mutation in the wild type M-MLV RT of SEQ ID NO: 33, or at a corresponding amino acid position in another wild type RT polypeptide sequence, wherein “X” can be any amino acid. In certain embodiments, X is N.

In various embodiments, the prime editors used in the complexes and methods described herein (with RT provided as either a fusion partner or in trans) can include a variant RT comprising an H204X mutation in the wild type M-MLV RT of SEQ ID NO: 33, or at a corresponding amino acid position in another wild type RT polypeptide sequence, wherein “X” can be any amino acid. In certain embodiments, X is R.

In various embodiments, the prime editors used in the complexes and methods described herein (with RT provided as either a fusion partner or in trans) can include a variant RT comprising an F209X mutation in the wild type M-MLV RT of SEQ ID NO: 33, or at a corresponding amino acid position in another wild type RT polypeptide sequence, wherein “X” can be any amino acid. In certain embodiments, X is N.

In various embodiments, the prime editors used in the complexes and methods described herein (with RT provided as either a fusion partner or in trans) can include a variant RT comprising an E302X mutation in the wild type M-MLV RT of SEQ ID NO: 33, or at a corresponding amino acid position in another wild type RT polypeptide sequence, wherein “X” can be any amino acid. In certain embodiments, X is K.

In various embodiments, the prime editors used in the complexes and methods described herein (with RT provided as either a fusion partner or in trans) can include a variant RT comprising an E302X mutation in the wild type M-MLV RT of SEQ ID NO: 33, or at a corresponding amino acid position in another wild type RT polypeptide sequence, wherein “X” can be any amino acid. In certain embodiments, X is R.

In various embodiments, the prime editors used in the complexes and methods described herein (with RT provided as either a fusion partner or in trans) can include a variant RT comprising a T306X mutation in the wild type M-MLV RT of SEQ ID NO: 33, or at a corresponding amino acid position in another wild type RT polypeptide sequence, wherein “X” can be any amino acid. In certain embodiments, X is K.

In various embodiments, the prime editors used in the complexes and methods described herein (with RT provided as either a fusion partner or in trans) can include a variant RT comprising an F309X mutation in the wild type M-MLV RT of SEQ ID NO: 33, or at a corresponding amino acid position in another wild type RT polypeptide sequence, wherein “X” can be any amino acid. In certain embodiments, X is N.

In various embodiments, the prime editors used in the complexes and methods described herein (with RT provided as either a fusion partner or in trans) can include a variant RT comprising a W313X mutation in the wild type M-MLV RT of SEQ ID NO: 33, or at a corresponding amino acid position in another wild type RT polypeptide sequence, wherein “X” can be any amino acid. In certain embodiments, X is F.

In various embodiments, the prime editors used in the complexes and methods described herein (with RT provided as either a fusion partner or in trans) can include a variant RT comprising a T330X mutation in the wild type M-MLV RT of SEQ ID NO: 33, or at a corresponding amino acid position in another wild type RT polypeptide sequence, wherein “X” can be any amino acid. In certain embodiments, X is P.

In various embodiments, the prime editors used in the complexes and methods described herein (with RT provided as either a fusion partner or in trans) can include a variant RT comprising an L345X mutation in the wild type M-MLV RT of SEQ ID NO: 33, or at a corresponding amino acid position in another wild type RT polypeptide sequence, wherein “X” can be any amino acid. In certain embodiments, X is G.

In various embodiments, the prime editors used in the complexes and methods described herein (with RT provided as either a fusion partner or in trans) can include a variant RT comprising an L435X mutation in the wild type M-MLV RT of SEQ ID NO: 33, or at a corresponding amino acid position in another wild type RT polypeptide sequence, wherein “X” can be any amino acid. In certain embodiments, X is G.

In various embodiments, the prime editors used in the complexes and methods described herein (with RT provided as either a fusion partner or in trans) can include a variant RT comprising an N454X mutation in the wild type M-MLV RT of SEQ ID NO: 33, or at a corresponding amino acid position in another wild type RT polypeptide sequence, wherein “X” can be any amino acid. In certain embodiments, X is K.

In various embodiments, the prime editors used in the complexes and methods described herein (with RT provided as either a fusion partner or in trans) can include a variant RT comprising a D524X mutation in the wild type M-MLV RT of SEQ ID NO: 33, or at a corresponding amino acid position in another wild type RT polypeptide sequence, wherein “X” can be any amino acid. In certain embodiments, X is G.

In various embodiments, the prime editors used in the complexes and methods described herein (with RT provided as either a fusion partner or in trans) can include a variant RT comprising an E562X mutation in the wild type M-MLV RT of SEQ ID NO: 33, or at a corresponding amino acid position in another wild type RT polypeptide sequence, wherein “X” can be any amino acid. In certain embodiments, X is Q.

In various embodiments, the prime editors used in the complexes and methods described herein (with RT provided as either a fusion partner or in trans) can include a variant RT comprising a D583X mutation in the wild type M-MLV RT of SEQ ID NO: 33, or at a corresponding amino acid position in another wild type RT polypeptide sequence, wherein “X” can be any amino acid. In certain embodiments, X is N.

In various embodiments, the prime editors used in the complexes and methods described herein (with RT provided as either a fusion partner or in trans) can include a variant RT comprising an H594X mutation in the wild type M-MLV RT of SEQ ID NO: 33, or at a corresponding amino acid position in another wild type RT polypeptide sequence, wherein “X” can be any amino acid. In certain embodiments, X is Q.

In various embodiments, the prime editors used in the complexes and methods described herein (with RT provided as either a fusion partner or in trans) can include a variant RT comprising an L603X mutation in the wild type M-MLV RT of SEQ ID NO: 33, or at a corresponding amino acid position in another wild type RT polypeptide sequence, wherein “X” can be any amino acid. In certain embodiments, X is W.

In various embodiments, the prime editors used in the complexes and methods described herein (with RT provided as either a fusion partner or in trans) can include a variant RT comprising an E607X mutation in the wild type M-MLV RT of SEQ ID NO: 33, or at a corresponding amino acid position in another wild type RT polypeptide sequence, wherein “X” can be any amino acid. In certain embodiments, X is K.

In various embodiments, the prime editors used in the complexes and methods described herein (with RT provided as either a fusion partner or in trans) can include a variant RT comprising a D653X mutation in the wild type M-MLV RT of SEQ ID NO: 33, or at a corresponding amino acid position in another wild type RT polypeptide sequence, wherein “X” can be any amino acid. In certain embodiments, X is N.

Some exemplary reverse transcriptases that can be fused to napDNAbp proteins or provided as individual proteins according to various embodiments of this disclosure are provided below. Exemplary reverse transcriptases include variants with at least 80%, at least 85%, at least 90%, at least 95%, at least 96%, at least 97%, at least 98%, or at least 99% sequence identity to the wild-type enzymes or partial enzymes described in SEQ ID NOs: 33-78, 159-179, and 188-213.

The prime editor (PE) system described here contemplates any publicly available reverse transcriptase (and any variant thereof) described or disclosed in any of the following U.S. patents (each of which is incorporated by reference), U.S. Pat. Nos. 10,202,658; 10,189,831; 10,150,955; 9,932,567; 9,783,791; 9,580,698; 9,534,201; and 9,458,484, that can be made using known methods for installing mutations or known methods for evolving proteins. The following references describe reverse transcriptases known in the art. Each of their disclosures are incorporated herein by reference.
Herzig, E., Voronin, N., Kucherenko, N. & Hizi, A. A Novel Leu92 Mutant of HIV-1 Reverse Transcriptase with a Selective Deficiency in Strand Transfer Causes a Loss of Viral Replication. J. Virol. 89, 8119-8129 (2015).
Mohr, G. et al. A Reverse Transcriptase-Cas1 Fusion Protein Contains a Cas6 Domain Required for Both CRISPR RNA Biogenesis and RNA Spacer Acquisition. Mol. Cell 72, 700-714.e8 (2018).
Zhao, C., Liu, F. & Pyle, A. M. An ultraprocessive, accurate reverse transcriptase encoded by a metazoan group II intron. RNA 24, 183-195 (2018).
Zimmerly, S. & Wu, L. An Unexplored Diversity of Reverse Transcriptases in Bacteria. Microbiol Spectr 3, MDNA3-0058-2014 (2015).
Ostertag, E. M. & Kazazian Jr, H. H. Biology of Mammalian L1 Retrotransposons. Annual Review of Genetics 35, 501-538 (2001).
Perach, M. & Hizi, A. Catalytic Features of the Recombinant Reverse Transcriptase of Bovine Leukemia Virus Expressed in Bacteria. Virology 259, 176-189 (1999).
Lim, D. et al. Crystal structure of the moloney murine leukemia virus RNase H domain. J. Virol. 80, 8379-8389 (2006).
Zhao, C. & Pyle, A. M. Crystal structures of a group II intron maturase reveal a missing link in spliceosome evolution. Nature Structural & Molecular Biology 23, 558-565 (2016).
Griffiths, D. J. Endogenous retroviruses in the human genome sequence. Genome Biol. 2, REVIEWS1017 (2001).
Baranauskas, A. et al., Generation and characterization of new highly thermostable and processive M-MuLV reverse transcriptase variants. Protein Eng Des Sel 25, 657-668 (2012).
Zimmerly, S., Guo, H., Perlman, P. S. & Lambowltz, A. M. Group II intron mobility occurs by target DNA-primed reverse transcription. Cell 82, 545-554 (1995).
Feng, Q., Moran, J. V., Kazazian, H. H. & Boeke, J. D. Human L1 retrotransposon encodes a conserved endonuclease required for retrotransposition. Cell 87, 905-916 (1996).
Berkhout, B., Jebbink, M. & Zsíros, J. Identification of an Active Reverse Transcriptase Enzyme Encoded by a Human Endogenous HERV-K Retrovirus. Journal of Virology 73, 2365-2375 (1999).
Kotewicz, M. L., Sampson, C. M., D'Alessio, J. M. & Gerard, G. F. Isolation of cloned Moloney murine leukemia virus reverse transcriptase lacking ribonuclease H activity. Nucleic Acids Res 16, 265-277 (1988).
Arezi, B. & Hogrefe, H. Novel mutations in Moloney Murine Leukemia Virus reverse transcriptase increase thermostability through tighter binding to template-primer. Nucleic Acids Res 37, 473-481 (2009).
Blain, S. W. & Goff, S. P. Nuclease activities of Moloney murine leukemia virus reverse transcriptase. Mutants with altered substrate specificities. J. Biol. Chem. 268, 23585-23592 (1993).
Xiong, Y. & Eickbush, T. H. Origin and evolution of retroelements based upon their reverse transcriptase sequences. EMBO J 9, 3353-3362 (1990).
Herschhorn, A. & Hizi, A. Retroviral reverse transcriptases. Cell. Mol. Life Sci. 67, 2717-2747 (2010).
Taube, R., Loya, S., Avidan, O., Perach, M. & Hizi, A. Reverse transcriptase of mouse mammary tumour virus: expression in bacteria, purification and biochemical characterization. Biochem. J. 329 (Pt 3), 579-587 (1998).
Liu, M. et al. Reverse Transcriptase-Mediated Tropism Switching in Bordetella Bacteriophage. Science 295, 2091-2094 (2002).
Luan, D. D., Korman, M. H., Jakubczak, J. L. & Eickbush, T. H. Reverse transcription of R2Bm RNA is primed by a nick at the chromosomal target site: a mechanism for non-LTR retrotransposition. Cell 72, 595-605 (1993).
Nottingham, R. M. et al. RNA-seq of human reference RNA samples using a thermostable group II intron reverse transcriptase. RNA 22, 597-613 (2016).
Telesnitsky, A. & Goff, S. P. RNase H domain mutations affect the interaction between Moloney murine leukemia virus reverse transcriptase and its primer-template. Proc. Natl. Acad. Sci. U.S.A. 90, 1276-1280 (1993).
Halvas, E. K., Svarovskaia, E. S. & Pathak, V. K. Role of Murine Leukemia Virus Reverse Transcriptase Deoxyribonucleoside Triphosphate-Binding Site in Retroviral Replication and In Vivo Fidelity. Journal of Virology 74, 10349-10358 (2000).
Nowak, E. et al., Structural analysis of monomeric retroviral reverse transcriptase in complex with an RNA/DNA hybrid. Nucleic Acids Res 41, 3874-3887 (2013).
Stamos, J. L., Lentzsch, A. M. & Lambowitz, A. M. Structure of a Thermostable Group II Intron Reverse Transcriptase with Template-Primer and Its Functional and Evolutionary Implications. Molecular Cell 68, 926-939.e4 (2017).
Das, D. & Georgiadis, M. M. The Crystal Structure of the Monomeric Reverse Transcriptase from Moloney Murine Leukemia Virus. Structure 12, 819-829 (2004).
Avidan, O., Meer, M. E., Oz, I. & Hizi, A. The processivity and fidelity of DNA synthesis exhibited by the reverse transcriptase of bovine leukemia virus. European Journal of Biochemistry 269, 859-867 (2002).
Gerard, G. F. et al. The role of template-primer in protection of reverse transcriptase from thermal inactivation. Nucleic Acids Res 30, 3118-3129 (2002).
Monot, C. et al. The Specificity and Flexibility of L1 Reverse Transcription Priming at Imperfect T-Tracts. PLOS Genetics 9, e1003499 (2013).
Mohr, S. et al. Thermostable group II intron reverse transcriptase fusion proteins and their use in cDNA synthesis and next-generation RNA sequencing. RNA 19, 958-970 (2013).

In some embodiments, the prime editor proteins comprise an MMLV reverse transcriptase comprising one or more amino acid substitutions. The wild-type MMLV reverse transcriptase is provided by the following sequence:

| DESCRIPTION | SEQUENCE |
|---|---|
| REVERSE | TLNIEDEYRLHETSKEPDVSLGSTWLSDFPQAWAETGGMGLA |
| TRANSCRIPTASE | VRQAPLIIPLKATSTPVSIKQYPMSQEARLGIKPHIQRLLDQGIL |
| (M-MLV RT) WILD | VPCQSPWNTPLLPVKKPGTNDYRPVQDLREVNKRVEDIHPTV |
| TYPE | PNPYNLLSGLPPSHQWYTVLDLKDAFFCLRLHPTSQPLFAFEW |
| MOLONEY MURINE | RDPEMGISGQLTWTRLPQGFKNSPTLFDEALHRDLADFRIQHP |
| LEUKEMIA VIRUS | DLILLQYVDDLLLAATSELDCQQGTRALLQTLGNLGYRASAK |
| | KAQICQKQVKYLGYLLKEGQRWLTEARKETVMGQPTPKTPR |
| | QLREFLGTAGFCRLWIPGFAEMAAPLYPLTKTGTLFNWGPDQ |
| | QKAYQEIKQALLTAPALGLPDLTKPFELFVDEKQGYAKGVLTQ |
| | KLGPWRRPVAYLSKKLDPVAAGWPPCLRMVAAIAVLTKDAGK |
| | LTMGQPLVILAPHAVEALVKQPPDRWLSNARMTHYQALLLDT |
| | DRVQFGPVVALNPATLLPLPEEGLQHNCLDILAEAHGTRPDLT |
| | DQPLPDADHTWYTDGSSLLQEGQRKAGAAVTTETEVIWAKA |
| | LPAGTSAQRAELIALTQALKMAEGKKLNVYTDSRYAFATAHIH |
| | GEIYRRRGLLTSEGKEIKNKDEILALLKALFLPKRLSIIHCPGHQ |
| | KGHSAEARGNRMADQAARKAAITETPDTSTLLIENSSP (SEQ |
| | ID NO: 33) |

The reverse transcriptases used in the prime editors described herein may comprise one or more mutations relative to the wild-type amino acid sequence. In some embodiments, the reverse transcriptase is the MMLV pentamutant described above (i.e., comprising amino acid substitutions D200N, T306K, W313F, T330P, and L603W).

In some embodiments, the present disclosure provides MMLV reverse transcriptase variants, and prime editors (e.g., fusion proteins and prime editors in which the napDNAbp and reverse transcriptase are provided in trans) comprising MMLV reverse transcriptase variants, wherein the variants comprise one or more mutations relative to SEQ ID NO: 33 selected from the group consisting of T13I, V19I, A32T, G38V, S60Y, P111L, K120R, H126Y, T128N, T128F, T128H, V129S, P132S, G138R, C157F, P175Q, P175S, D200S, D200Y, D200N, D200C, Y222F, V223A, V223M, V223T, V223W, V223Y, L234I, T246I, N249S, T287A, P292T, E302A, E302K, T306K, G316R, E346K, K373N, W388C, V402A, K445N, M457I, and A462S. In some embodiments, an MMLV reverse transcriptase variant comprises two or more of these mutations, three or more of these mutations, four or more of these mutations, or five or more of these mutations.

In some embodiments, the MMLV reverse transcriptase variants used in the prime editors provided herein comprise a single mutation relative to SEQ ID NO: 33. In some embodiments, the single mutations is selected from the group consisting of T13I, G38V, K120R, H126Y, T128N, T128F, T128H, V129S, P132S, P175Q, P175S, D200C, D200Y, V223M, V223T, V223W, V223Y, L234I, P292T, G316R, K373N, M457I, and V402A.

In certain embodiments, the MMLV reverse transcriptase variants used in the prime editors provided herein comprise any one of the following groups of mutations relative to the amino acid sequence of SEQ ID NO: 33: D200Y and E302A; D200Y, V223A, and M457I; V223M, T306K, and A462S; D200N and E302K; D200Y and E302K; T128N and V223A; V19I, A32T, and D200Y; D200S, V223A, E346K, and W388C; S60Y, V223A, and N249S; P111L, V223A, T287A, and G316R; S60Y, G138R, and V223A; S60Y, Y222F, V223A, and K445N; or S60Y, C157F, V223A, and T246I. In certain embodiments, the MMLV reverse transcriptase variant used in the prime editors provided herein comprises the amino acid sequence of any one of SEQ ID NOs: 33-42, 63-78, and 172-179, or an amino acid sequence at least 70%, at least 75%, at least 80%, at least 85%, at least 90%, at least 95%, at least 96%, at least 97%, at least 98%, or at least 99% identical to any one of SEQ ID NOs: 33-42, 63-78, and 172-179, wherein the amino acid sequence comprises at least one of residues 13I, 19I, 32T, 38V, 60Y, 111L, 120R, 126Y, 128N, 128F, 128H, 129S, 132S, 138R, 157F, 175Q, 175S, 200S, 200Y, 200N, 200C, 222F, 223A, 223M, 223T, 223W, 223Y, 234I, 246I, 249S, 287A, 292T, 302A, 302K, 306K, 316R, 346K, 373N, 388C, 402A, 445N, 457I, and 462S.

In other examples, the proteins described herein may comprise an MMLV reverse transcriptase comprising one or more substitutions at amino acid positions V19, A32, S60, P111, T128, G138R, C157F, D200, Y222, V223, T246, N249, T287, G316, E346, W388, and/or K445. In some embodiments, the proteins described herein comprise an MMLV reverse transcriptase comprising one or more substitutions selected from the group consisting of V19I, A32T, S60Y, P111L, T128N, G138R, C157F, D200S, D200Y, Y222F, V223A, T246I, N249S, T287A, G316R, E346K, W388C, and K445N. In certain embodiments, the proteins described herein comprise an MMLV reverse transcriptase comprising any one of the following groups of amino acid substitutions:
T128N and V223A;
V19I, A32T, and D200Y;
D200S, V223A, E346K, and W388C;
S60Y, V223A, and N249S;
P111L, V223A, T287A, and G316R;
S60Y, G138R, and V223A;
S60Y, Y222F, V223A, and K445N; or
S60Y, C157F, V223A, and T246I.

Exemplary evolved reverse transcriptase enzymes are as follows:

| DESCRIPTION | SEQUENCE |
|---|---|
| REVERSE | TLNIEDEYRLHETSKEPDVSLGSTWLSDFPQAWAETGGMGLA |
| TRANSCRIPTASE | VRQAPLIIPLKATSTPVSIKQYPMSQEARLGIKPHIQRLLDQGIL |
| (M-MLV RT) | VPCQSPWNTPLLPVKKPGTNDYRPVQDLREVNKRVEDIHPNV |
| T128N and V223A | PNPYNLLSGLPPSHQWYTVLDLKDAFFCLRLHPTSQPLFAFEW |
| | RDPEMGISGQLTWTRLPQGFKNSPTLFDEALHRDLADFRIQHP |
| | DLILLQYADDLLLAATSELDCQQGTRALLQTLGNLGYRASAK |
| | KAQICQKQVKYLGYLLKEGQRWLTEARKETVMGQPTPKTPR |
| | QLREFLGTAGFCRLWIPGFAEMAAPLYPLTKTGTLFNWGPDQ |
| | QKAYQEIKQALLTAPALGLPDLTKPFELFVDEKQGYAKGVLTQ |
| | KLGPWRRPVAYLSKKLDPVAAGWPPCLRMVAAIAVLTKDAGK |
| | LTMGQPLVILAPHAVEALVKQPPDRWLSNARMTHYQALLLDT |
| | DRVQFGPVVALNPATLLPLPEEGLQHNCLDILAEAHGTRPDLT |
| | DQPLPDADHTWYTDGSSLLQEGQRKAGAAVTTETEVIWAKA |
| | LPAGTSAQRAELIALTQALKMAEGKKLNVYTDSRYAFATAHIH |
| | GEIYRRRGLLTSEGKEIKNKDEILALLKALFLPKRLSIIHCPGHQ |
| | KGHSAEARGNRMADQAARKAAITETPDTSTLLIENSSP (SEQ |
| | ID NO: 35) |
| REVERSE | TLNIEDEYRLHETSKEPDISLGSTWLSDFPQTWAETGGMGLAV |
| TRANSCRIPTASE | RQAPLIIPLKATSTPVSIKQYPMSQEARLGIKPHIQRLLDQGILV |
| (M-MLV RT) | PCQSPWNTPLLPVKKPGTNDYRPVQDLREVNKRVEDIHPTVP |
| V19I, A32T, and | NPYNLLSGLPPSHQWYTVLDLKDAFFCLRLHPTSQPLFAFEW |
| D200Y | RDPEMGISGQLTWTRLPQGFKNSPTLFYEALHRDLADFRIQHP |
| | DLILLQYVDDLLLAATSELDCQQGTRALLQTLGNLGYRASAK |
| | KAQICQKQVKYLGYLLKEGQRWLTEARKETVMGQPTPKTPR |
| | QLREFLGTAGFCRLWIPGFAEMAAPLYPLTKTGTLFNWGPDQ |
| | QKAYQEIKQALLTAPALGLPDLTKPFELFVDEKQGYAKGVLTQ |
| | KLGPWRRPVAYLSKKLDPVAAGWPPCLRMVAAIAVLTKDAGK |
| | LTMGQPLVILAPHAVEALVKQPPDRWLSNARMTHYQALLLDT |
| | DRVQFGPVVALNPATLLPLPEEGLQHNCLDILAEAHGTRPDLT |
| | DQPLPDADHTWYTDGSSLLQEGQRKAGAAVTTETEVIWAKA |
| | LPAGTSAQRAELIALTQALKMAEGKKLNVYTDSRYAFATAHIH |
| | GEIYRRRGLLTSEGKEIKNKDEILALLKALFLPKRLSIIHCPGHQ |
| | KGHSAEARGNRMADQAARKAAITETPDTSTLLIENSSP (SEQ |
| | ID NO: 36) |
| REVERSE | TLNIEDEYRLHETSKEPDVSLGSTWLSDFPQAWAETGGMGLA |
| TRANSCRIPTASE | VRQAPLIIPLKATSTPVSIKQYPMSQEARLGIKPHIQRLLDQGIL |
| (M-MLV RT) | VPCQSPWNTPLLPVKKPGTNDYRPVQDLREVNKRVEDIHPTV |
| D200S, V223A, | PNPYNLLSGLPPSHQWYTVLDLKDAFFCLRLHPTSQPLFAFEW |
| E346K, and W388C | RDPEMGISGQLTWTRLPQGFKNSPTLFSEALHRDLADFRIQHP |
| | DLILLQYADDLLLAATSELDCQQGTRALLQTLGNLGYRASAK |
| | KAQICQKQVKYLGYLLKEGQRWLTEARKETVMGQPTPKTPR |
| | QLREFLGTAGFCRLWIPGFAEMAAPLYPLTKTGTLFNWGPDQ |
| | QKAYQKIKQALLTAPALGLPDLTKPFELFVDEKQGYAKGVLTQ |
| | KLGPCRRPVAYLSKKLDPVAAGWPPCLRMVAAIAVLTKDAGK |
| | LTMGQPLVILAPHAVEALVKQPPDRWLSNARMTHYQALLLDT |
| | DRVQFGPVVALNPATLLPLPEEGLQHNCLDILAEAHGTRPDLT |
| | DQPLPDADHTWYTDGSSLLQEGQRKAGAAVTTETEVIWAKA |
| | LPAGTSAQRAELIALTQALKMAEGKKLNVYTDSRYAFATAHIH |
| | GEIYRRRGLLTSEGKEIKNKDEILALLKALFLPKRLSIIHCPGHQ |
| | KGHSAEARGNRMADQAARKAAITETPDTSTLLIENSSP (SEQ |
| | ID NO: 37) |
| REVERSE | TLNIEDEYRLHETSKEPDVSLGSTWLSDFPQAWAETGGMGLA |
| TRANSCRIPTASE | VRQAPLIIPLKATSTPVYIKQYPMSQEARLGIKPHIQRLLDQGIL |
| (M-MLV RT) | VPCQSPWNTPLLPVKKPGTNDYRPVQDLREVNKRVEDIHPTV |
| S60Y, V223A, and | PNPYNLLSGLPPSHQWYTVLDLKDAFFCLRLHPTSQPLFAFEW |
| N249S | RDPEMGISGQLTWTRLPQGFKNSPTLFDEALHRDLADFRIQHP |
| | DLILLQYADDLLLAATSELDCQQGTRALLQTLGSLGYRASAK |
| | KAQICQKQVKYLGYLLKEGQRWLTEARKETVMGQPTPKTPR |
| | QLREFLGTAGFCRLWIPGFAEMAAPLYPLTKTGTLFNWGPDQ |
| | QKAYQEIKQALLTAPALGLPDLTKPFELFVDEKQGYAKGVLTQ |
| | KLGPWRRPVAYLSKKLDPVAAGWPPCLRMVAAIAVLTKDAGK |
| | LTMGQPLVILAPHAVEALVKQPPDRWLSNARMTHYQALLLDT |
| | DRVQFGPVVALNPATLLPLPEEGLQHNCLDILAEAHGTRPDLT |
| | DQPLPDADHTWYTDGSSLLQEGQRKAGAAVTTETEVIWAKA |
| | LPAGTSAQRAELIALTQALKMAEGKKLNVYTDSRYAFATAHIH |
| | GEIYRRRGLLTSEGKEIKNKDEILALLKALFLPKRLSIIHCPGHQ |
| | KGHSAEARGNRMADQAARKAAITETPDTSTLLIENSSP (SEQ |
| | ID NO: 38) |
| REVERSE | TLNIEDEYRLHETSKEPDVSLGSTWLSDFPQAWAETGGMGLA |
| TRANSCRIPTASE | VRQAPLIIPLKATSTPVSIKQYPMSQEARLGIKPHIQRLLDQGIL |
| (M-MLV RT) | VPCQSPWNTPLLPVKKPGTNDYRLVQDLREVNKRVEDIHPTV |
| P111L, V223A, | PNPYNLLSGLPPSHQWYTVLDLKDAFFCLRLHPTSQPLFAFEW |
| T287A, and G316R | RDPEMGISGQLTWTRLPQGFKNSPTLFDEALHRDLADFRIQHP |
| | DLILLQYADDLLLAATSELDCQQGTRALLQTLGNLGYRASAK |
| | KAQICQKQVKYLGYLLKEGQRWLTEARKEAVMGQPTPKTPR |
| | QLREFLGTAGFCRLWIPRFAEMAAPLYPLTKTGTLFNWGPDQ |
| | QKAYQEIKQALLTAPALGLPDLTKPFELFVDEKQGYAKGVLTQ |
| | KLGPWRRPVAYLSKKLDPVAAGWPPCLRMVAAIAVLTKDAGK |
| | LTMGQPLVILAPHAVEALVKQPPDRWLSNARMTHYQALLLDT |
| | DRVQFGPVVALNPATLLPLPEEGLQHNCLDILAEAHGTRPDLT |
| | DQPLPDADHTWYTDGSSLLQEGQRKAGAAVTTETEVIWAKA |
| | LPAGTSAQRAELIALTQALKMAEGKKLNVYTDSRYAFATAHIH |
| | GEIYRRRGLLTSEGKEIKNKDEILALLKALFLPKRLSIIHCPGHQ |
| | KGHSAEARGNRMADQAARKAAITETPDTSTLLIENSSP (SEQ |
| | ID NO: 39) |
| REVERSE | TLNIEDEYRLHETSKEPDVSLGSTWLSDFPQAWAETGGMGLA |
| TRANSCRIPTASE | VRQAPLIIPLKATSTPVYIKQYPMSQEARLGIKPHIQRLLDQGIL |
| (M-MLV RT) | VPCQSPWNTPLLPVKKPGTNDYRPVQDLREVNKRVEDIHPTV |
| S60Y, G138R, and | PNPYNLLSRLPPSHQWYTVLDLKDAFFCLRLHPTSQPLFAFEW |
| V223A | RDPEMGISGQLTWTRLPQGFKNSPTLFDEALHRDLADFRIQHP |
| | DLILLQYADDLLLAATSELDCQQGTRALLQTLGNLGYRASAK |
| | KAQICQKQVKYLGYLLKEGQRWLTEARKETVMGQPTPKTPR |
| | QLREFLGTAGFCRLWIPGFAEMAAPLYPLTKTGTLFNWGPDQ |
| | QKAYQEIKQALLTAPALGLPDLTKPFELFVDEKQGYAKGVLTQ |
| | KLGPWRRPVAYLSKKLDPVAAGWPPCLRMVAAIAVLTKDAGK |
| | LTMGQPLVILAPHAVEALVKQPPDRWLSNARMTHYQALLLDT |
| | DRVQFGPVVALNPATLLPLPEEGLQHNCLDILAEAHGTRPDLT |
| | DQPLPDADHTWYTDGSSLLQEGQRKAGAAVTTETEVIWAKA |
| | LPAGTSAQRAELIALTQALKMAEGKKLNVYTDSRYAFATAHIH |
| | GEIYRRRGLLTSEGKEIKNKDEILALLKALFLPKRLSIIHCPGHQ |
| | KGHSAEARGNRMADQAARKAAITETPDTSTLLIENSSP (SEQ |
| | ID NO: 40) |
| REVERSE | TLNIEDEYRLHETSKEPDVSLGSTWLSDFPQAWAETGGMGLA |
| TRANSCRIPTASE | VRQAPLIIPLKATSTPVYIKQYPMSQEARLGIKPHIQRLLDQGIL |
| (M-MLV RT) | VPCQSPWNTPLLPVKKPGTNDYRPVQDLREVNKRVEDIHPTV |
| S60Y, Y222F, V223A, | PNPYNLLSGLPPSHQWYTVLDLKDAFFCLRLHPTSQPLFAFEW |
| and K445N | RDPEMGISGQLTWTRLPQGFKNSPTLFDEALHRDLADFRIQHP |
| | DLILLQFADDLLLAATSELDCQQGTRALLQTLGNLGYRASAK |
| | KAQICQKQVKYLGYLLKEGQRWLTEARKETVMGQPTPKTPR |
| | QLREFLGTAGFCRLWIPGFAEMAAPLYPLTKTGTLFNWGPDQ |
| | QKAYQEIKQALLTAPALGLPDLTKPFELFVDEKQGYAKGVLTQ |
| | KLGPWRRPVAYLSKKLDPVAAGWPPCLRMVAAIAVLTKDAGK |
| | LTMGQPLVILAPHAVEALVNQPPDRWLSNARMTHYQALLLDT |
| | DRVQFGPVVALNPATLLPLPEEGLQHNCLDILAEAHGTRPDLT |
| | DQPLPDADHTWYTDGSSLLQEGQRKAGAAVTTETEVIWAKA |
| | LPAGTSAQRAELIALTQALKMAEGKKLNVYTDSRYAFATAHIH |
| | GEIYRRRGLLTSEGKEIKNKDEILALLKALFLPKRLSIIHCPGHQ |
| | KGHSAEARGNRMADQAARKAAITETPDTSTLLIENSSP (SEQ |
| | ID NO: 41) |
| REVERSE | TLNIEDEYRLHETSKEPDVSLGSTWLSDFPQAWAETGGMGLA |
| TRANSCRIPTASE | VRQAPLIIPLKATSTPVYIKQYPMSQEARLGIKPHIQRLLDQGIL |
| (M-MLV RT) | VPCQSPWNTPLLPVKKPGTNDYRPVQDLREVNKRVEDIHPTV |
| S60Y, C157F, V223A, | PNPYNLLSGLPPSHQWYTVLDLKDAFFFLRLHPTSQPLFAFEW |
| and T246I | RDPEMGISGQLTWTRLPQGFKNSPTLFDEALHRDLADFRIQHP |
| | DLILLQYADDLLLAATSELDCQQGTRALLQILGNLGYRASAK |
| | KAQICQKQVKYLGYLLKEGQRWLTEARKETVMGQPTPKTPR |
| | QLREFLGTAGFCRLWIPGFAEMAAPLYPLTKTGTLFNWGPDQ |
| | QKAYQEIKQALLTAPALGLPDLTKPFELFVDEKQGYAKGVLTQ |
| | KLGPWRRPVAYLSKKLDPVAAGWPPCLRMVAAIAVLTKDAGK |
| | LTMGQPLVILAPHAVEALVKQPPDRWLSNARMTHYQALLLDT |
| | DRVQFGPVVALNPATLLPLPEEGLQHNCLDILAEAHGTRPDLT |
| | DQPLPDADHTWYTDGSSLLQEGQRKAGAAVTTETEVIWAKA |
| | LPAGTSAQRAELIALTQALKMAEGKKLNVYTDSRYAFATAHIH |
| | GEIYRRRGLLTSEGKEIKNKDEILALLKALFLPKRLSIIHCPGHQ |
| | KGHSAEARGNRMADQAARKAAITETPDTSTLLIENSSP (SEQ |
| | ID NO: 42) |
| REVERSE | TLNIEDEYRLHETSKEPDVSLGSTWLSDFPQAWAETGGMGLA |
| TRANSCRIPTASE | VRQAPLIIPLKATSTPVSIKQYPMSQEARLGIKPHIQRLLDQGIL |
| (M-MLV RT) | VPCQSPWNTPLLPVKKPGTNDYRPVQDLREVNKRVEDIHPTV |
| D200S, V223A, | PNPYNLLSGLPPSHQWYTVLDLKDAFFCLRLHPTSQPLFAFEW |
| E346K, and W388C | RDPEMGISGQLTWTRLPQGFKNSPTLFSEALHRDLADFRIQHP |
| | DLILLQYADDLLLAATSELDCQQGTRALLQTLGNLGYRASAK |
| | KAQICQKQVKYLGYLLKEGQRWLTEARKETVMGQPTPKTPR |
| | QLREFLGKAGFCRLFIPGFAEMAAPLYPLTKPGTLFNWGPDQ |
| | QKAYQKIKQALLTAPALGLPDLTKPFELFVDEKQGYAKGVLT |
| | QKLGPWCRPVAYLSKKLDPVAAGWPPCLRMVAAIAVLTKDA |
| | GKLTMGQPLVILAPHAVEALVKQPPDRWLSNARMTHYQALL |
| | LDTDRVQFGPVVALNPATLLPLPEEGLQHNCLD (SEQ ID NO: |
| | 172) |
| REVERSE | TLNIEDEYRLHETSKEPDVSLGSTWLSDFPQAWAETGGMGLA |
| TRANSCRIPTASE | VRQAPLIIPLKATSTPVYIKQYPMSQEARLGIKPHIQRLLDQGIL |
| (M-MLV RT) | VPCQSPWNTPLLPVKKPGTNDYRPVQDLREVNKRVEDIHPTV |
| S60Y, V223A, and | PNPYNLLSGLPPSHQWYTVLDLKDAFFCLRLHPTSQPLFAFEW |
| N249S | RDPEMGISGQLTWTRLPQGFKNSPTLFNEALHRDLADFRIQHP |
| | DLILLQYADDLLLAATSELDCQQGTRALLQTLGSLGYRASAK |
| | KAQICQKQVKYLGYLLKEGQRWLTEARKETVMGQPTPKTPR |
| | QLREFLGKAGFCRLFIPGFAEMAAPLYPLTKPGTLFNWGPDQ |
| | QKAYQEIKQALLTAPALGLPDLTKPFELFVDEKQGYAKGVLT |
| | QKLGPWRRPVAYLSKKLDPVAAGWPPCLRMVAAIAVLTKDA |
| | GKLTMGQPLVILAPHAVEALVKQPPDRWLSNARMTHYQALL |
| | LDTDRVQFGPVVALNPATLLPLPEEGLQHNCLD (SEQ ID NO: |
| | 173) |
| REVERSE | TLNIEDEYRLHETSKEPDVSLGSTWLSDFPQAWAETGGMGLA |
| TRANSCRIPTASE | VRQAPLIIPLKATSTPVSIKQYPMSQEARLGIKPHIQRLLDQGIL |
| (M-MLV RT) | VPCQSPWNTPLLPVKKPGTNDYRLVQDLREVNKRVEDIHPTV |
| P111L, V223A, | PNPYNLLSGLPPSHQWYTVLDLKDAFFCLRLHPTSQPLFAFEW |
| T287A, and G316R | RDPEMGISGQLTWTRLPQGFKNSPTLFNEALHRDLADFRIQHP |
| | DLILLQYADDLLLAATSELDCQQGTRALLQTLGNLGYRASAK |
| | KAQICQKQVKYLGYLLKEGQRWLTEARKEAVMGQPTPKTPR |
| | QLREFLGKAGFCRLFIPRFAEMAAPLYPLTKPGTLFNWGPDQ |
| | QKAYQEIKQALLTAPALGLPDLTKPFELFVDEKQGYAKGVLT |
| | QKLGPWRRPVAYLSKKLDPVAAGWPPCLRMVAAIAVLTKDA |
| | GKLTMGQPLVILAPHAVEALVKQPPDRWLSNARMTHYQALL |
| | LDTDRVQFGPVVALNPATLLPLPEEGLQHNCLD (SEQ ID NO: |
| | 174) |
| REVERSE | TLNIEDEYRLHETSKEPDVSLGSTWLSDFPQAWAETGGMGLA |
| TRANSCRIPTASE | VRQAPLIIPLKATSTPVYIKQYPMSQEARLGIKPHIQRLLDQGIL |
| (M-MLV RT) | VPCQSPWNTPLLPVKKPGTNDYRPVQDLREVNKRVEDIHPTV |
| S60Y, G138R, and | PNPYNLLSRLPPSHQWYTVLDLKDAFFCLRLHPTSQPLFAFEW |
| V223A | RDPEMGISGQLTWTRLPQGFKNSPTLFNEALHRDLADFRIQHP |
| | DLILLQYADDLLLAATSELDCQQGTRALLQTLGNLGYRASAK |
| | KAQICQKQVKYLGYLLKEGQRWLTEARKETVMGQPTPKTPR |
| | QLREFLGKAGFCRLFIPGFAEMAAPLYPLTKPGTLFNWGPDQ |
| | QKAYQEIKQALLTAPALGLPDLTKPFELFVDEKQGYAKGVLT |
| | QKLGPWRRPVAYLSKKLDPVAAGWPPCLRMVAAIAVLTKDA |
| | GKLTMGQPLVILAPHAVEALVKQPPDRWLSNARMTHYQALL |
| | LDTDRVQFGPVVALNPATLLPLPEEGLQHNCLD (SEQ ID NO: |
| | 175) |
| REVERSE | TLNIEDEYRLHETSKEPDVSLGSTWLSDFPQAWAETGGMGLA |
| TRANSCRIPTASE | VRQAPLIIPLKATSTPVYIKQYPMSQEARLGIKPHIQRLLDQGIL |
| (M-MLV RT) | VPCQSPWNTPLLPVKKPGTNDYRPVQDLREVNKRVEDIHPTV |
| S60Y, Y222F, V223A, | PNPYNLLSGLPPSHQWYTVLDLKDAFFCLRLHPTSQPLFAFEW |
| and K445N | RDPEMGISGQLTWTRLPQGFKNSPTLFNEALHRDLADFRIQHP |
| | DLILLQFADDLLLAATSELDCQQGTRALLQTLGNLGYRASAK |
| | KAQICQKQVKYLGYLLKEGQRWLTEARKETVMGQPTPKTPR |
| | QLREFLGKAGFCRLFIPGFAEMAAPLYPLTKPGTLFNWGPDQ |
| | QKAYQEIKQALLTAPALGLPDLTKPFELFVDEKQGYAKGVLT |
| | QKLGPWRRPVAYLSKKLDPVAAGWPPCLRMVAAIAVLTKDA |
| | GKLTMGQPLVILAPHAVEALVNQPPDRWLSNARMTHYQALL |
| | LDTDRVQFGPVVALNPATLLPLPEEGLQHNCLD (SEQ ID NO: |
| | 176) |
| REVERSE | TLNIEDEYRLHETSKEPDVSLGSTWLSDFPQAWAETGGMGLA |
| TRANSCRIPTASE | VRQAPLIIPLKATSTPVYIKQYPMSQEARLGIKPHIQRLLDQGIL |
| (M-MLV RT) | VPCQSPWNTPLLPVKKPGTNDYRPVQDLREVNKRVEDIHPTV |
| S60Y, C157F, V223A, | PNPYNLLSGLPPSHQWYTVLDLKDAFFFLRLHPTSQPLFAFEW |
| and T246I | RDPEMGISGQLTWTRLPQGFKNSPTLFNEALHRDLADFRIQHP |
| | DLILLQYADDLLLAATSELDCQQGTRALLQILGNLGYRASAK |
| | KAQICQKQVKYLGYLLKEGQRWLTEARKETVMGQPTPKTPR |
| | QLREFLGKAGFCRLFIPGFAEMAAPLYPLTKPGTLFNWGPDQ |
| | QKAYQEIKQALLTAPALGLPDLTKPFELFVDEKQGYAKGVLT |
| | QKLGPWRRPVAYLSKKLDPVAAGWPPCLRMVAAIAVLTKDA |
| | GKLTMGQPLVILAPHAVEALVKQPPDRWLSNARMTHYQALL |
| | LDTDRVQFGPVVALNPATLLPLPEEGLQHNCLD (SEQ ID NO: |
| | 177) |
| REVERSE | TLNIEDEYRLHETSKEPDVSLGSTWLSDFPQAWAETGGMGLA |
| TRANSCRIPTASE | VRQAPLIIPLKATSTPVSIKQYPMSQEARLGIKPHIQRLLDQGIL |
| (M-MLV RT) | VPCQSPWNTPLLPVKKPGTNDYRPVQDLREVNKRVEDIHPTV |
| V223M, T306K, | PNPYNLLSGLPPSHQWYTVLDLKDAFFCLRLHPTSQPLFAFEW |
| A462S | RDPEMGISGQLTWTRLPQGFKNSPTLFNEALHRDLADFRIQHP |
| | DLILLQYMDDLLLAATSELDCQQGTRALLQTLGNLGYRASAK |
| | KAQICQKQVKYLGYLLKEGQRWLTEARKETVMGQPTPKTPR |
| | QLREFLGKAGFCRLFIPGFAEMAAPLYPLTKPGTLFNWGPDQ |
| | QKAYQEIKQALLTAPALGLPDLTKPFELFVDEKQGYAKGVLT |
| | QKLGPWRRPVAYLSKKLDPVAAGWPPCLRMVAAIAVLTKDA |
| | GKLTMGQPLVILAPHAVEALVKQPPDRWLSNARMTHYQSLL |
| | LDTDRVQFGPVVALNPATLLPLPEEGLQHNCLD (SEQ ID NO: |
| | 178) |
| REVERSE | TLNIEDEYRLHETSKEPDVSLGSTWLSDFPQAWAETGGMGLA |
| TRANSCRIPTASE | VRQAPLIIPLKATSTPVSIKQYPMSQEARLGIKPHIQRLLDQGIL |
| (M-MLV RT) | VPCQSPWNTPLLPVKKPGTNDYRPVQDLREVNKRVEDIHPTV |
| D200N and E302K | PNPYNLLSGLPPSHQWYTVLDLKDAFFCLRLHPTSQPLFAFEW |
| | RDPEMGISGQLTWTRLPQGFKNSPTLFNEALHRDLADFRIQHP |
| | DLILLQYVDDLLLAATSELDCQQGTRALLQTLGNLGYRASAK |
| | KAQICQKQVKYLGYLLKEGQRWLTEARKETVMGQPTPKTPR |
| | QLRKFLGKAGFCRLFIPGFAEMAAPLYPLTKPGTLFNWGPDQ |
| | QKAYQEIKQALLTAPALGLPDLTKPFELFVDEKQGYAKGVLT |
| | QKLGPWRRPVAYLSKKLDPVAAGWPPCLRMVAAIAVLTKDA |
| | GKLTMGQPLVILAPHAVEALVKQPPDRWLSNARMTHYQALL |
| | LDTDRVQFGPVVALNPATLLPLPEEGLQHNCLD (SEQ ID NO: |
| | 179) |

The use of reverse transcriptase enzymes comprising an amino acid sequence having at least 80%, at least 85%, at least 90%, at least 95%, or at least 99% sequence identity to any of the evolved variants described herein in the prime editors disclosed herein is also contemplated by the present disclosure, provided the RT sequence comprises one of the amino acid substitutions disclosed herein.

The disclosure also contemplates the use of any wild-type reverse transcriptase in the prime editors described herein. Exemplary wild-type reverse transcriptases which may be used include, but are not limited to, the following sequences, or any variant thereof having at least 80%, at least 85%, at least 90%, at least 95%, or at least 99% sequence identity thereto:

| | | SEQ |
|---|---|---|
| | | ID |
| DESCRIPTION | SEQUENCE | NO: |
| MOUSE | VFTLWGRDIMKDIKVRLMTDSPDDSQDLMIGAIESNLFADQIS | 43 |
| MAMMARY | WKSDQPVWLNQWPLKQEKLQALQQLVTEQLQLGHLEESNSP | |
| TUMOR VIRUS | WNTPVFVIKKKSGKWRLLQDLRAVNATMHDMGALQPGLPSP | |
| (MMTV) | VAVPKGWEIIIIDLQDCFFNIKLHPEDCKRFAFSVPSPNFKRPYQ | |
| REVERSE | RFQWKVLPQGMKNSPTLCQKFVDKAILTVRDKYQDSYIVHY | |
| TRANSCRIPTASE | MDDILLAHPSRSIVDEILTSMIQALNKHGLVVSTEKIQKYDNL | |
| | KYLGTHIQGDSVSYQKLQIRTDKLRTLNDFQKLLGNINWIRPF | |
| | LKLTTGELKPLFEILNGDSNPISTRKLTPEACKALQLMNERLST | |
| | ARVKRLDLSQPWSLCILKTEYTPTACLWQDGVVEWIHLPHISP | |
| | KVITPYDIFCTQLIIKGRHRSKELFSKDPDYIVVPYTKVQFDLLL | |
| | QEKEDWPISLLGFLGEVHFHLPKDPLLTFTLQTAIIFPHMTSTTP | |
| | LEKGIVIFTDGSANGRSVTYIQGREPIIKENTQNTAQQAEIVAVI | |
| | TAFEEVSQPFNLYTDSKYVTGLFPEIETATLSPRTKIYTELKHL | |
| | QRLIHKRQEKFYIGHIRGHTGLPGPLAQGNAYADSLTRILT | |
| AVIAN | TVALHLAIPLKWKPDHTPVWIDQWPLPEGKLVALTQLVEKEL | 44 |
| SARCOMA | QLGHIEPSLSCWNTPVFVIRKASGSYRLLHDLRAVNAKLVPFG | |
| LEUKOSIS VIRUS | AVQQGAPVLSALPRGWPLMVLDLKDCFFSIPLAEQDREAFAF | |
| (ASLV) REVERSE | TLPSVNNQAPARRFQWKVLPQGMTCSPTICQLVVGQVLEPLR | |
| TRANSCRIPTASE | LKHPSLRMLHYMDDLLLAASSHDGLEAAGEEVISTLERAGFTI | |
| | SPDKIQREPGVQYLGYKLGSTYVAPVGLVAEPRIATLWDVQK | |
| | LVGSLQWLRPALGIPPRLMGPFYEQLRGSDPNEAREWNLDMK | |
| | MAWREIVQLSTTAALERWDPALPLEGAVARCEQGAIGVLGQ | |
| | GLSTHPRPCLWLFSTQPTKAFTAWLEVLTLLITKLRASAVRTF | |
| | GKEVDILLLPACFREDLPLPEGILLALKGFAGKIRSSDTPSIFDIA | |
| | RPLHVSLKVRVTDHPVPGPTVFTDASSSTHKGVVVWREGPRW | |
| | EIKEIADSGASVQQLEARAVAMALLLWPTTPTNVVTDSAFVA | |
| | KMLLKMGQEGVPSTAAAFILEDALSQRSAMAAVLHVRSHSE | |
| | VPGFFTEGNDVADSQATFQAY | |
| PORCINE | TLQLDDEYRLYSPQVKPDQDIQSWLEQFPQAWAETAGMGLA | 45 |
| ENDOGENOUS | KQVPPQVIQLKASATPVSVRQYPLSREAREGIWPHVQRLIQQG | |
| RETROVIRUS | ILVPVQSPWNTPLLPVRKPGTNDYRPVQDLREVNKRVQDIHPT | |
| (PERV) REVERSE | VPNPYNLLSALPPERNWYTVLDLKDAFFCLRLHPTSQPLFAFE | |
| TRANSCRIPTASE | WRDPGTGRTGQLTWTRLPQGFKNSPTIFDEALHRDLANFRIQ | |
| | HPQVTLLQYVDDLLLAGATKQDCLEGTKALLLELSDLGYRAS | |
| | AKKAQICRREVTYLGYSLRGGQRWLTEARKKTVVQIPAPTTA | |
| | KQVREFLGTAGFCRLWIPGFATLAAPLYPLTKEKGEFSWAPEH | |
| | QKAFDAIKKALLSAPALALPDVTKPFTLYVDERKGVARGVLT | |
| | QTLGPWRRPVAYLSKKLDPVASGWPVCLKAIAAVAILVKDA | |
| | DKLTLGQNITVIAPHALENIVRQPPDRWMTNARMTHYQSLLL | |
| | TERVTFAPPAALNPATLLPEETDEPVTHDCHQLLIEETGVRKD | |
| | LTDIPLTGEVLTWFTDGSSYVVEGKRMAGAAVVDGTHTIWAS | |
| | SLPEGTSAQKAELMALTQALRLAEGKSINIYTDSRYAFATAHV | |
| | HGAIYKQRGLLTSAGREIKNKEEILSLLEALHLPKRLAIIHCPG | |
| | HQKAKDLISRGNQMADRVAKQAAQAVNLLPI | |
| HIV-MMLV | PISPIETVPVKLKPGMDGPKVKQWPLTEEKIKALVEICTEMEKE | 46 |
| REVERSE | GKISKIGPENPYNTPVFAIKKKDSTKWRKLVDFRELNKRTQDF | |
| TRANSCRIPTASE | WEVQLGIPHPAGLKKKKSVTVLDVGDAYFSVPLDEDFRKYTA | |
| | FTIPSINNETPGIRYQYNVLPQGWKGSPAIFQSSMTKILEPFKKQ | |
| | NPDIVIYQYMDDLYVGSDLEIGQHRTKIEELRQHLLRWGLTTP | |
| | DKKHQKEPPFLWMGYELHPDKWTVQPIVLPEKDSWTVNDIQ | |
| | KLVGKLNWASQIYPGIKVRQLCKLLRGTKALTEVIPLTEEAEL | |
| | ELAENREILKEPVHGVYYDPSKDLIAEIQKQGQGQWTYQIYQE | |
| | PFKNLKTGKYARMRGAHTNDVKQLTEAVQKITTESIVIWGKT | |
| | PKFKLPIQKETWETWWTEYWQATWIPEWEFVNTPPLVKLVV | |
| | ALNPATLLPLPEEGLQHNCLDILAEAHGTRPDLTDQPLPDADH | |
| | TWYTDGSSLLQEGQRKAGAAVTTETEVIWAKALPAGTSAQR | |
| | AELIALTQALKMAEGKKLNVYTDSRYAFATAHIHGEIYRRRG | |
| | WLTSEGKEIKNKDEILALLKALFLPKRLSIIHCPGHQKGHSAEA | |
| | RGNRMADQAARKAAITETPDTSTLLIEN | |
| AVIAN | APLEEEYRLFLEAPIQNVTLLEQWKREIPKVWAEINPPGLASTQ | 47 |
| RETICULOENDO | APIHVQLLSTALPVRVRQYPITLEAKRSLRETIRKFRAAGILRP | |
| THELIOSIS | VHSPWNTPLLPVRKSGTSEYRMVQDLREVNKRVETIHPTVPN | |
| VIRUS (AVIRE) | PYTLLSLLPPDRIWYSVLDLKDAFFCIPLAPESQLIFAFEWADA | |
| REVERSE | EEGESGQLTWTRLPQGFKNSPTLFDEALNRDLQGFRLDHPSVS | |
| TRANSCRIPTASE | LLQYVDDLLIAADTQAACLSATRDLLMTLAELGYRVSGKKA | |
| | QLCQEEVTYLGFKIHKGSRSLSNSRTQAILQIPVPKTKRQVREF | |
| | LGTIGYCRLWIPGFAELAQPLYAATRGGNDPLVWGEKEEEAF | |
| | QSLKLALTQPPALALPSLDKPFQLFVEETSGAAKGVLTQALGP | |
| | WKRPVAYLSKRLDPVAAGWPRCLRAIAAAALLTREASKLTFG | |
| | QDIEITSSHNLESLLRSPPDKWLTNARITQYQVLLLDPPRVRFK | |
| | QTAALNPATLLPETDDTLPIHHCLDTLDSLTSTRPDLTDQPLAQ | |
| | AEATLFTDGSSYIRDGKRYAGAAVVTLDSVIWAEPLPIGTSAQ | |
| | KAELIALTKALEWSKDKSVNIYTDSRYAFATLHVHGMIYRER | |
| | GLLTAGGKAIKNAPEILALLTAVWLPKRVAVMHCKGHQKDD | |
| | APTSTGNRRADEVAREVAIRPLSTQATISDAPDMPDTETPQYS | |
| | NVEEALG | |
| BABOON | VSLQDEHRLFDIPVTTSLPDVWLQDFPQAWAETGGLGRAKCQ | 48 |
| ENDOGENOUS | APIIIDLKPTAVPVSIKQYPMSLEAHMGIRQHIIKFLELGVLRPC | |
| VIRUS (BAEVM) | RSPWNTPLLPVKKPGTQDYRPVQDLREINKRTVDIHPTVPNPY | |
| REVERSE | NLLSTLKPDYSWYTVLDLKDAFFCLPLAPQSQELFAFEWKDP | |
| TRANSCRIPTASE | ERGISGQLTWTRLPQGFKNSPTLFDEALHRDLTDFRTQHPEVT | |
| | LLQYVDDLLLAAPTKKACTQGTRHLLQELGEKGYRASAKKA | |
| | QICQTKVTYLGYILSEGKRWLTPGRIETVARIPPPRNPREVREF | |
| | LGTAGFCRLWIPGFAELAAPLYALTKESTPFTWQTEHQLAFEA | |
| | LKKALLSAPALGLPDTSKPFTLFLDERQGIAKGVLTQKLGPWK | |
| | RPVAYLSKKLDPVAAGWPPCLRIMAATAMLVKDSAKLTLGQ | |
| | PLTVITPHTLEAIVRQPPDRWITNARLTHYQALLLDTDRVQFG | |
| | PPVTLNPATLLPVPENQPSPHDCRQVLAETHGTREDLKDQELP | |
| | DADHTWYTDGSSYLDSGTRRAGAAVVDGHNTIWAQSLPPGT | |
| | SAQKAELIALTKALELSKGKKANIYTDSRYAFATAHTHGSIYE | |
| | RRGLLTSEGKEIKNKAEIIALLKALFLPQEVAIIHCPGHQKGQD | |
| | PVAVGNRQADRVARQAAMAEVLTLATEPDNTSHITIEHTYTS | |
| | EDQEEA | |
| GIBBON APE | LNLEEEYRLHEKPVPSSIDPSWLQLFPTVWAERAGMGLANQV | 49 |
| LEUKEMIA | PPVVVELRSGASPVAVRQYPMSKEAREGIRPHIQKFLDLGVLV | |
| VIRUS (GALV) | PCRSPWNTPLLPVKKPGTNDYRPVQDLREINKRVQDIHPTVPN | |
| REVERSE | PYNLLSSLPPSYTWYSVLDLKDAFFCLRLHPNSQPLFAFEWKD | |
| TRANSCRIPTASE | PEKGNTGQLTWTRLPQGFKNSPTLFDEALHRDLAPFRALNPQ | |
| | VVLLQYVDDLLVAAPTYEDCKKGTQKLLQELSKLGYRVSAK | |
| | KAQLCQREVTYLGYLLKEGKRWLTPARKATVMKIPVPTTPRQ | |
| | VREFLGTAGFCRLWIPGFASLAAPLYPLTKESIPFIWTEEHQQA | |
| | FDHIKKALLSAPALALPDLTKPFTLYIDERAGVARGVLTQTLG | |
| | PWRRPVAYLSKKLDPVASGWPTCLKAVAAVALLLKDADKLT | |
| | LGQNVTVIASHSLESIVRQPPDRWMTNARMTHYQSLLLNERV | |
| | SFAPPAVLNPATLLPVESEATPVHRCSEILAEETGTRRDLEDQP | |
| | LPGVPTWYTDGSSFITEGKRRAGAPIVDGKRTVWASSLPEGTS | |
| | AQKAELVALTQALRLAEGKNINIYTDSRYAFATAHIHGAIYKQ | |
| | RGLLTSAGKDIKNKEEILALLEAIHLPRRVAIIHCPGHQRGSNP | |
| | VATGNRRADEAAKQAALSTRVLAGTTKPQEPIEPAQEK | |
| KOALA | MNLEEEYRLHEKPVPPSIDPSWLQLFPMVWAEKAGMGLANQ | 50 |
| RETROVIRUS | VPPVVVELKSDASPVAVRQYPMSKEAREGIRPHIQRFLDLGIL | |
| (KORV) REVERSE | VPCQSPWNTPLLPVKKPGTNDYRPVQDLREVNKRVQDIHPTV | |
| TRANSCRIPTASE | PNPYNLLSSLPPSHTWYSVLDLKDAFFCLKLHPNSQPLFAFEW | |
| | RDPEKGNTGQLTWTRLPQGFKNSPTLFDEALHRDLASFRALN | |
| | PQVVMLQYVDDLLVAAPTYRDCKEGTRRLLQELSKLGYRVS | |
| | AKKAQLCREEVTYLGYLLKGGKRWLTPARKATVMKIPTPTTP | |
| | RQVREFLGTAGFCRLWIPGFASLAAPLYPLTREKVPFTWTEAH | |
| | QEAFGRIKEALLSAPALALPDLTKPFALYVDEKEGVARGVLTQ | |
| | TLGPWRRPVAYLSKKLDPVASGWPTCLKAIAAVALLLKDAD | |
| | KLTLGQNVLVIAPHNLESIVRQPPDRWMTNARMTHYQSLLLN | |
| | ERVSFAPPAILNPATLLPVESDDTPIHICSEILAEETGTRPDLRD | |
| | QPLPGVPAWYTDGSSFIMDGRRQAGAAIVDNKRTVWASNLPE | |
| | GTSAQKAELIALTQALRLAEGKSINIYTDSRYAFATAHVHGAI | |
| | YKQRGLLTSAGKDIKNKEEILALLEAIHLPKRVAIIHCPGHQRG | |
| | TDPVATGNRKADEAAKQAAQSTRILTETTKNQEHFEPTRGK | |
| MASON-PFIZER | MWGRDLLSQMKIMMCSPNDIVTAQMLAQGYSPGKGLGKKE | 51 |
| MONKEY VIRUS | NGILHPIPNQGQSNKKGFGNFLTAAIDILAPQQCAEPITWKSDE | |
| (MPMV) | PVWVDQWPLTNDKLAAAQQLVQEQLEAGHITESSSPWNTPIF | |
| REVERSE | VIKKKSGKWRLLQDLRAVNATMVLMGALQPGLPSPVAIPQG | |
| TRANSCRIPTASE | YLKIIIDLKDCFFSIPLHPSDQKRFAFSLPSTNFKEPMQRFQWK | |
| | VLPQGMANSPTLCQKYVATAIHKVRHAWKQMYIIHYMDDILI | |
| | AGKDGQQVLQCFDQLKQELTAAGLHIAPEKVQLQDPYTYLGF | |
| | ELNGPKITNQKAVIRKDKLQTLNDFQKLLGDINWLRPYLKLTT | |
| | GDLKPLFDTLKGDSDPNSHRSLSKEALASLEKVETAIAEQFVT | |
| | HINYSLPLIFLIFNTALTPTGLFWQDNPIMWIHLPASPKKVLLPY | |
| | YDAIADLIILGRDHSKKYFGIEPSTIIQPYSKSQIDWLMQNTEM | |
| | WPIACASFVGILDNHYPPNKLIQFCKLHTFVFPQIISKTPLNNAL | |
| | LVFTDGSSTGMAAYTLTDTTIKFQTNLNSAQLVELQALIAVLS | |
| | AFPNQPLNIYTDSAYLAHSIPLLETVAQIKHISETAKLFLQCQQ | |
| | LIYNRSIPFYIGHVRAHSGLPGPIAQGNQRADLATKIVA | |
| POK11ERV | ATVEPPKPIPLTWKTEKPVWVNQWPLPKQKLEALHLLANEQL | 52 |
| REVERSE | EKGHIEPSFSPWNSPVFVIQKKSGKWRMLTDLRAVNAVIQPM | |
| TRANSCRIPTASE | GPLQPGLPSPAMIPKDWPLIIIDLKDCFFTIPLAEQDCEKFAFTIP | |
| | AINNKEPATRFQWKVLPQGMLNSPTICQTFVGRALQPVREKFS | |
| | DCYIIHYIDDILCAAETKDKLIDCYTFLQAEVANAGLAIASDKI | |
| | QTSTPFHYLGMQIENRKIKPQKIEIRKDTLKTLNDFQKLLGDIN | |
| | WIRPTLGIPTYAMSNLFSILRGDSDLNSKRILTPEATKEIKLVEE | |
| | KIQSAQINRIDPLAPLQLLIFATAHSPTGIIIQNTDLVEWSFLPHS | |
| | TVKTFTLYLDQIATLIGQTRLRIIKLCGNDPDKIVVPLTKEQVR | |
| | QAFINSGAWQIGLANFVGIIDNHYPKTKIFQFLKMTTWILPKIT | |
| | RREPLENALTVFTDGSSNGKAAYTGPKERVIKTPYQSAQRAEL | |
| | VAVITVLQDFDQPINIISDSAYVVQATRDVETALIKYSMDDQL | |
| | NQLFNLLQQTVRKRNFPFYITHIRAHTNLPGPLTKANEEADLL | |
| | VS | |
| SIMIAN | MWGRDLLSQMKIMMCSPNDIVTAQMLAQGYSPGKGLGKRE | 53 |
| RETROVIRUS | DGILQPIPNSGQLDRKGFGNFLATAVDILAPQRYADPITWKSD | |
| TYPE 2 (SRV2) | EPVWVDQWPLTQEKLAAAQQLVQEQLQAGHIIESNSPWNTPI | |
| REVERSE | FVIKKKSGKWRLLQDLRAVNATMVLMGALQPGLPSPVAIPQG | |
| TRANSCRIPTASE | YFKIVIDLKDCFFTIPLQPVDQKRFAFSLPSTNFKQPMKRYQW | |
| | KVLPQGMANSPTLCQKYVAAAIEPVRKSWAQMYIIHYMDDIL | |
| | IAGKLGEQVLQCFAQLKQALTTTGLQIAPEKVQLQDPYTYLGF | |
| | QINGPKITNQKAVIRRDKLQTLNDFQKLLGDINWLRPYLHLTT | |
| | GDLKPLFDILKGDSNPNSPRSLSEAALASLQKVETAIAEQFVTQ | |
| | IDYTQPLTFLIFNTTLTPTGLFWQNNPVMWVHLPASPKKVLLP | |
| | YYDAIADLIILGRDNSKKYFGLEPSTIIQPYSKSQIHWLMQNTE | |
| | TWPIACASYAGNIDNHYPPNKLIQFCKLHAVVFPRIISKTPLDN | |
| | ALLVFTDGSSTGIAAYTFEKTTVRFKTSHTSAQLVELQALIAVL | |
| | SAFPHRALNVYTDSAYLAHSIPLLETVSHIKHISDTAKFFLQCQ | |
| | QLIYNRSIPFYLGHIRAHSGLPGPLSQGNHITDLATKVVA | |
| WOOLLY | LNLEEEYRLHEKPVPSSIDPSWLQLFPTVWAERAGMGLANQV | 54 |
| MONKEY | PPVVVELRSGASPVAVRQYPMSKEAREGIRPHIQRFLDLGVLV | |
| SARCOMA VIRUS | PCQSPWNTPLLPVKKPGTNDYRPVQDLREINKRVQDIHPTVPN | |
| (WMSV) REVERSE | PYNLLSSLPPSHTWYSVLDLKDAFFCLKLHPNSQPLFAFEWRD | |
| TRANSCRIPTASE | PEKGNTGQLTWTRLPQGFKNSPTLFDEALHRDLAPFRALNPQ | |
| | VVLLQYVDDLLVAAPTYRDCKEGTQKLLQELSKLGYRVSAK | |
| | KAQLCQKEVTYLGYLLKEGKRWLTPARKATVMKIPPPTTPRQ | |
| | VREFLGTAGFCRLWIPGFASLAAPLYPLTKESIPFIWTEEHQKA | |
| | FDRIKEALLSAPALALPDLTKPFTLYVDERAGVARGVLTQTLG | |
| | PWRRPVAYLSKKLDPVASGWPTCLKAVAAVALLLKDADKLT | |
| | LGQNVTVIASHSLESIVRQPPDRWMTNARMTHYQSLLLNERV | |
| | SFAPPAVLNPATLLPVESEATPVHRCSEILAEETGTRRDLKDQP | |
| | LPGVPAWYTDGSSFIAEGKRRAGAAIVDGKRTVWASSLPEGT | |
| | SAQKAELVALTQALRLAEGKDINIYTDSRYAFATAHIHGAIYK | |
| | QRGLLTSAGKDIKNKEEILALLEAIHLPKRVAIIHCPGHQKGND | |
| | PVATGNRRADEAAKQAALSTRVLAETTKPQELI | |
| TF1 REVERSE | ISSSKHTLSQMNKVSNIVKEPELPDIYKEFKDITADTNTEKLPK | 55 |
| TRANSCRIPTASE | PIKGLEFEVELTQENYRLPIRNYPLPPGKMQAMNDEINQGLKS | |
| | GIIRESKAINACPVMFVPKKEGTLRMVVDYKPLNKYVKPNIYP | |
| | LPLIEQLLAKIQGSTIFTKLDLKSAYHLIRVRKGDEHKLAFRCP | |
| | RGVFEYLVMPYGISTAPAHFQYFINTILGEAKESHVVCYMDDI | |
| | LIHSKSESEHVKHVKDVLQKLKNANLIINQAKCEFHQSQVKFI | |
| | GYHISEKGFTPCQENIDKVLQWKQPKNRKELRQFLGSVNYLR | |
| | KFIPKTSQLTHPLNKLLKKDVRWKWTPTQTQAIENIKQCLVSP | |
| | PVLRHFDFSKKILLETDASDVAVGAVLSQKHDDDKYYPVGYY | |
| | SAKMSKAQLNYSVSDKEMLAIIKSLKHWRHYLESTIEPFKILT | |
| | DHRNLIGRITNESEPENKRLARWQLFLQDFNFEINYRPGSANHI | |
| | ADALSRIVDETEPIPKDSEDNSINFVNQISI | |
| CRISPR REVERSE | NSQAQSACCAGANQIVEGATLEKVVAPACLQQAWTRVRKNK | 56 |
| TRANSCRIPTASE | GGPGGDGVTIEIFAQNAEVELEKLRAETLAGIYRPRKVRHAIV | |
| | PKPKGGERKLTIPSVVDRILQTATMLSLGQTVDHHESSASWAY | |
| | REGRGVDDALADLRRLRNSGLFWTFDADIMQYFDRILHKRLI | |
| | DDLFIWVDDLRIVRLIQLWLRSFSYWGRGIAQGAPISPLLANLF | |
| | LHPMDRLLELEGLASVRYADDFVVLCRSKALAQKAQLIVASH | |
| | LAARGLKLNMSKTRILAPSEAFIFLGQTVEPVWDTQP | |
| VP96 REVERSE | NLVKRLAHHLGKSEPEVIHFLADAPNKYRVYKIPKRSYGHRVI | 57 |
| TRANSCRIPTASE | AQPTRELKLYQKAFLELYSFPVHSSATAYCKGKSIKDNALSHV | |
| | KNHYLLKTDLENFFNSITPNIFWKSIENDSIATPKFSTSEIALVE | |
| | RLIFWRPSKLQGGKLVLSVGAPSSPTISNFCLYQFDEYLSIICKE | |
| | QNISYTRYADDLTFSTCDKDVLHTVIPLIQSLLDYFFASELKLN | |
| | HSKTVFSSKAHNRHVTGITLNNEGKLSLGRERKRYIKHLVHSF | |
| | KYGKLDNTEIRHLQGMLSFAKHIEPIFIDRLKEKYTDELIKIIYE | |
| | AGHE | |
| VC95 REVERSE | NILTTLREQLLTNNVIMPQEFERLEVRGSHAYKVYSIPKRKAG | 58 |
| TRANSCRIPTASE | RRTIAHPSSKLKICQRHLNAILNPLLKVHDSSYAYVKGRSIKDN | |
| | ALVHSHSAYVLKMDFQNFFNSITPTILRQCLIQNDILLSVNELE | |
| | KLEQLIFWNPSKKRNGKLILSVGSPISPLISNAIMYPFDKIINDIC | |
| | TKHGINYTRYADDITESTNIKNTLNKLPEIVEQLIIQTYAGRIIIN | |
| | KRKTVFSSKKHNRHVTGITLTNDSKISIGRSRKRYISSLVFKYIN | |
| | KNLDIDEINHMKGMLAFAYNIEPIYIHRLSHKYKVNIVEKILRG | |
| | SN | |
| EC48 REVERSE | GRPYVTLNLNGMFMDKFKPYSKSNAPITTLEKLSKALSISVEE | 59 |
| TRANSCRIPTASE | LKAIAELSLDEKYTLKEIPKIDGSKRIVYSLHPKMRLLQSRINK | |
| | RIFKELVVFPSFLFGSVPSKNDVLNSNVKRDYVSCAKAHCGA | |
| | KTVLKVDISNFFDNIHRDLVRSVFEEILHIKDEALEYLVDICTK | |
| | DDFVVQGALTSSYIATLCLFAVEGDVVRRAQRKGLVYTRLVD | |
| | DITVSSKISNYDFSQMQSHIERMLSEHDLPINKHKTKIFHCSSEP | |
| | IKVHGLRVDYDSPRLPSDEVKRIRASIHNLKLLAAKNNTKTSV | |
| | AYRKEFNRCMGRVNKLGRVGHEKYESFKKQLQAIKPMPSKR | |
| | DVAVIDAAIKSLELSYSKGNQNKHWYKRKYDLTRYKMIILTR | |
| | SESFKEKLECFKSRLASLKPL | |
| GS REVERSE | ALLERILARDNLITALKRVEANQGAPGIDGVSTDQLRDYIRAH | 60 |
| TRANSCRIPTASE | WSTIHAQLLAGTYRPAPVRRVEIPKPGGGTRQLGIPTVVDRLI | |
| | QQAILQELTPIFDPDFSSSSFGFRPGRNAHDAVRQAQGYIQEGY | |
| | RYVVDMDLEKFFDRVNHDILMSRVARKVKDKRVLKLIRAYL | |
| | QAGVMIEGVKVQTEEGTPQGGPLSPLLANILLDDLDKELEKR | |
| | GLKFCRYADDCNIYVKSLRAGQRVKQSIQRFLEKTLKLKVNE | |
| | EKSAVDRPWKRAFLGFSFTPERKARIRLAPRSIQRLKQRIRQLT | |
| | NPNWSISMPERIHRVNQYVMGWIGYFRLVETPSVLQTIEGWIR | |
| | RRLRLCQWLQWKRVRTRIRELRALGLKETAVMEIANTRKGA | |
| | WRTTKTPQLHQALGKTYWTAQGLKSLTQRYFELRQG | |
| ER REVERSE | DTSNLMEQILSSDNLNRAYLQVVRNKGAEGVDGMKYTELKE | 61 |
| TRANSCRIPTASE | HLAKNGETIKGQLRTRKYKPQPARRVEIPKPDGGVRNLGVPT | |
| | VTDRFIQQAIAQVLTPIYEEQFHDHSYGFRPNRCAQQAILTALN | |
| | IMNDGNDWIVDIDLEKFFDTVNHDKLMTLIGRTIKDGDVISIV | |
| | RKYLVSGIMIDDEYEDSIVGTPQGGNLSPLLANIMLNELDKEM | |
| | EKRGLNFVRYADDCIIMVGSEMSANRVMRNISRFIEEKLGLKV | |
| | NMTKSKVDRPSGLKYLGFGFYFDPRAHQFKAKPHAKSVAKF | |
| | KKRMKELTCRSWGVSNSYKVEKLNQLIRGWINYFKIGSMKTL | |
| | CKELDSRIRYRLRMCIWKQWKTPQNQEKNLVKLGIDRNTARR | |
| | VAYTGKRIAYVCNKGAVNVAISNKRLASFGLISMLDYYIEKC | |
| | VTC | |
| NE144 REVERSE | AGQPTSREALYERIRSTSKEEVILEEMIRLGFWPAQGAVPHDP | 62 |
| TRANSCRIPTASE | AEEIRRRGELERQLSELREKSRKLYNEKALIAEQRKQRLAESR | |
| | RKQKETKARRERERQERAQKWAQRKAGEILFLGEDVSGGMS | |
| | HKTCDAELIKREGVPAIASAEELARAMGIALKELRFLAYNRKV | |
| | SRVTHYRRFLLPKKTGGLRLISAPMPRLKRAQAWALEHIFNKL | |
| | SFEPAAHGFVAGRSIVSNARPHVGADVVVNLDLKDFFPTVSFP | |
| | RVKGALRHLGYSESVATALALVCTEPEVDEVGLDGTTWYVA | |
| | RGERFLPQGSPCSPAITNLLCRRLDRRLHGLAQALGFVYTRYA | |
| | DDLTFSGRGEAAESKRVGKLLRGAADIVAHEGFVVHPDKTRV | |
| | MRRGRRQEVTGVVVNDKTSVPRDELRKFRATLYQIEKDGPA | |
| | DKRWGNGGDVLAAVHGYACFVAMVDPSRGQPLLARARALL | |
| | AKHGGPSKPPGGSGPRAPTPVQPTANAPEAPKPVAPATPAAPA | |
| | KKGWKLF | |

The use of reverse transcriptase enzymes comprising an amino acid sequence having at least 80%, at least 85%, at least 90%, at least 95%, or at least 99% sequence identity to any of the enzymes above in the prime editor proteins disclosed herein is also contemplated by the present disclosure.

In some embodiments, the present disclosure provides reverse transcriptases, and prime editors (e.g. fusion proteins or prime editors in which each component is provided in trans) comprising reverse transcriptases, wherein the reverse transcriptase is an AVIRE reverse transcriptase of SEQ ID NO: 47, or an AVIRE reverse transcriptase variant having at least 70%, at least 75%, at least 80%, at least 85%, at least 90%, at least 95%, at least 96%, at least 97%, at least 98%, or at least 99% sequence identity with SEQ ID NO: 47, wherein the AVIRE reverse transcriptase variant comprises one or more mutations selected from the group consisting of D199N, T305K, W312F, G329P, and L604W. In some embodiments, the AVIRE reverse transcriptase variant comprises two or more, three or more, four or more, or all five of these mutations. In some embodiments, the AVIRE reverse transcriptase variant comprises the mutation D199N. In some embodiments, the AVIRE reverse transcriptase variant comprises the mutation T305K. In some embodiments, the AVIRE reverse transcriptase variant comprises the mutation W312F. In some embodiments, the AVIRE reverse transcriptase variant comprises the mutation G329P. In some embodiments, the AVIRE reverse transcriptase variant comprises the mutation L604W.

In certain embodiments, the AVIRE reverse transcriptase variant comprises the amino acid sequence of any one of SEQ ID NOs: 214-219, or an amino acid sequence at least 70%, at least 75%, at least 80%, at least 85%, at least 90%, at least 95%, at least 96%, at least 97%, at least 98%, or at least 99% identical to any one of SEQ ID NOs: 214-219, wherein the amino acid sequence comprises at least one of the residues 199N, 305K, 312F, 329P, and 604W:

AVIRE-RT (D199N):
(SEQ ID NO: 214)
APLEEEYRLFLEAPIQNVTLLEQWKREIPKVWAEINPPGLASTQAPIHVQLLSTALPVR
VRQYPITLEAKRSLRETIRKFRAAGILRPVHSPWNTPLLPVRKSGTSEYRMVQDLREV
NKRVETIHPTVPNPYTLLSLLPPDRIWYSVLDLKDAFFCIPLAPESQLIFAFEWADAEE
GESGQLTWTRLPQGFKNSPTLFNEALNRDLQGFRLDHPSVSLLQYVDDLLIAADTQA
ACLSATRDLLMTLAELGYRVSGKKAQLCQEEVTYLGFKIHKGSRSLSNSRTQAILQIP
VPKTKRQVREFLGTIGYCRLWIPGFAELAQPLYAATRGGNDPLVWGEKEEEAFQSLK
LALTQPPALALPSLDKPFQLFVEETSGAAKGVLTQALGPWKRPVAYLSKRLDPVAA
GWPRCLRAIAAAALLTREASKLTFGQDIEITSSHNLESLLRSPPDKWLTNARITQYQV
LLLDPPRVRFKQTAALNPATLLPETDDTLPIHHCLDTLDSLTSTRPDLTDQPLAQAEA
TLFTDGSSYIRDGKRYAGAAVVTLDSVIWAEPLPIGTSAQKAELIALTKALEWSKDK
SVNIYTDSRYAFATLHVHGMIYRERGLLTAGGKAIKNAPEILALLTAVWLPKRVAV
MHCKGHQKDDAPTSTGNRRADEVAREVAIRPLSTQATISDAPDMPDTETPQYSNVE
EALG
AVIRE-RT (T305K):
(SEQ ID NO: 215)
APLEEEYRLFLEAPIQNVTLLEQWKREIPKVWAEINPPGLASTQAPIHVQLLSTALPVR
VRQYPITLEAKRSLRETIRKFRAAGILRPVHSPWNTPLLPVRKSGTSEYRMVQDLREV
NKRVETIHPTVPNPYTLLSLLPPDRIWYSVLDLKDAFFCIPLAPESQLIFAFEWADAEE
GESGQLTWTRLPQGFKNSPTLFDEALNRDLQGFRLDHPSVSLLQYVDDLLIAADTQA
ACLSATRDLLMTLAELGYRVSGKKAQLCQEEVTYLGFKIHKGSRSLSNSRTQAILQIP
VPKTKRQVREFLGKIGYCRLWIPGFAELAQPLYAATRGGNDPLVWGEKEEEAFQSL
KLALTQPPALALPSLDKPFQLFVEETSGAAKGVLTQALGPWKRPVAYLSKRLDPVA
AGWPRCLRAIAAAALLTREASKLTFGQDIEITSSHNLESLLRSPPDKWLTNARITQYQ
VLLLDPPRVRFKQTAALNPATLLPETDDTLPIHHCLDTLDSLTSTRPDLTDQPLAQAE
ATLFTDGSSYIRDGKRYAGAAVVTLDSVIWAEPLPIGTSAQKAELIALTKALEWSKD
KSVNIYTDSRYAFATLHVHGMIYRERGLLTAGGKAIKNAPEILALLTAVWLPKRVAV
MHCKGHQKDDAPTSTGNRRADEVAREVAIRPLSTQATISDAPDMPDTETPQYSNVE
EALG
AVIRE-RT (W312F):
(SEQ ID NO: 216)
APLEEEYRLFLEAPIQNVTLLEQWKREIPKVWAEINPPGLASTQAPIHVQLLSTALPVR
VRQYPITLEAKRSLRETIRKFRAAGILRPVHSPWNTPLLPVRKSGTSEYRMVQDLREV
NKRVETIHPTVPNPYTLLSLLPPDRIWYSVLDLKDAFFCIPLAPESQLIFAFEWADAEE
GESGQLTWTRLPQGFKNSPTLFDEALNRDLQGFRLDHPSVSLLQYVDDLLIAADTQA
ACLSATRDLLMTLAELGYRVSGKKAQLCQEEVTYLGFKIHKGSRSLSNSRTQAILQIP
VPKTKRQVREFLGTIGYCRLFIPGFAELAQPLYAATRGGNDPLVWGEKEEEAFQSLK
LALTQPPALALPSLDKPFQLFVEETSGAAKGVLTQALGPWKRPVAYLSKRLDPVAA
GWPRCLRAIAAAALLTREASKLTFGQDIEITSSHNLESLLRSPPDKWLTNARITQYQV
LLLDPPRVRFKQTAALNPATLLPETDDTLPIHHCLDTLDSLTSTRPDLTDQPLAQAEA
TLFTDGSSYIRDGKRYAGAAVVTLDSVIWAEPLPIGTSAQKAELIALTKALEWSKDK
SVNIYTDSRYAFATLHVHGMIYRERGLLTAGGKAIKNAPEILALLTAVWLPKRVAV
MHCKGHQKDDAPTSTGNRRADEVAREVAIRPLSTQATISDAPDMPDTETPQYSNVE
EALG
AVIRE-RT (G329P):
(SEQ ID NO: 217)
APLEEEYRLFLEAPIQNVTLLEQWKREIPKVWAEINPPGLASTQAPIHVQLLSTALPVR
VRQYPITLEAKRSLRETIRKFRAAGILRPVHSPWNTPLLPVRKSGTSEYRMVQDLREV
NKRVETIHPTVPNPYTLLSLLPPDRIWYSVLDLKDAFFCIPLAPESQLIFAFEWADAEE
GESGQLTWTRLPQGFKNSPTLFDEALNRDLQGFRLDHPSVSLLQYVDDLLIAADTQA
ACLSATRDLLMTLAELGYRVSGKKAQLCQEEVTYLGFKIHKGSRSLSNSRTQAILQIP
VPKTKRQVREFLGTIGYCRLWIPGFAELAQPLYAATRPGNDPLVWGEKEEEAFQSLK
LALTQPPALALPSLDKPFQLFVEETSGAAKGVLTQALGPWKRPVAYLSKRLDPVAA
GWPRCLRAIAAAALLTREASKLTFGQDIEITSSHNLESLLRSPPDKWLTNARITQYQV
LLLDPPRVRFKQTAALNPATLLPETDDTLPIHHCLDTLDSLTSTRPDLTDQPLAQAEA
TLFTDGSSYIRDGKRYAGAAVVTLDSVIWAEPLPIGTSAQKAELIALTKALEWSKDK
SVNIYTDSRYAFATLHVHGMIYRERGLLTAGGKAIKNAPEILALLTAVWLPKRVAV
MHCKGHQKDDAPTSTGNRRADEVAREVAIRPLSTQATISDAPDMPDTETPQYSNVE
EALG
AVIRE-RT (L604W):
(SEQ ID NO: 218)
APLEEEYRLFLEAPIQNVTLLEQWKREIPKVWAEINPPGLASTQAPIHVQLLSTALPVR
VRQYPITLEAKRSLRETIRKFRAAGILRPVHSPWNTPLLPVRKSGTSEYRMVQDLREV
NKRVETIHPTVPNPYTLLSLLPPDRIWYSVLDLKDAFFCIPLAPESQLIFAFEWADAEE
GESGQLTWTRLPQGFKNSPTLFDEALNRDLQGFRLDHPSVSLLQYVDDLLIAADTQA
ACLSATRDLLMTLAELGYRVSGKKAQLCQEEVTYLGFKIHKGSRSLSNSRTQAILQIP
VPKTKRQVREFLGTIGYCRLWIPGFAELAQPLYAATRGGNDPLVWGEKEEEAFQSLK
LALTQPPALALPSLDKPFQLFVEETSGAAKGVLTQALGPWKRPVAYLSKRLDPVAA
GWPRCLRAIAAAALLTREASKLTFGQDIEITSSHNLESLLRSPPDKWLTNARITQYQV
LLLDPPRVRFKQTAALNPATLLPETDDTLPIHHCLDTLDSLTSTRPDLTDQPLAQAEA
TLFTDGSSYIRDGKRYAGAAVVTLDSVIWAEPLPIGTSAQKAELIALTKALEWSKDK
SVNIYTDSRYAFATLHVHGMIYRERGWLTAGGKAIKNAPEILALLTAVWLPKRVAV
MHCKGHQKDDAPTSTGNRRADEVAREVAIRPLSTQATISDAPDMPDTETPQYSNVE
EALG

In certain embodiments, the AVIRE reverse transcriptase variant comprises an amino acid sequence of SEQ ID NO: 219, or an amino acid sequence at least 70%, at least 75%, at least 80%, at least 85%, at least 90%, at least 95%, at least 96%, at least 97%, at least 98%, or at least 99% identical to SEQ ID NO: 219, wherein the amino acid sequence comprises the residues 199N, 305K, 312F, 329P, and 604W:

AVIRE_penta:
(SEQ ID NO: 219)
APLEEEYRLFLEAPIQNVTLLEQWKREIPKVWAEINPPGLASTQAPIHVQLLSTALPVR
VRQYPITLEAKRSLRETIRKFRAAGILRPVHSPWNTPLLPVRKSGTSEYRMVQDLREV
NKRVETIHPTVPNPYTLLSLLPPDRIWYSVLDLKDAFFCIPLAPESQLIFAFEWADAEE
GESGQLTWTRLPQGFKNSPTLFNEALNRDLQGFRLDHPSVSLLQYVDDLLIAADTQA
ACLSATRDLLMTLAELGYRVSGKKAQLCQEEVTYLGFKIHKGSRSLSNSRTQAILQIP
VPKTKRQVREFLGKIGYCRLFIPGFAELAQPLYAATRPGNDPLVWGEKEEEAFQSLK
LALTQPPALALPSLDKPFQLFVEETSGAAKGVLTQALGPWKRPVAYLSKRLDPVAA
GWPRCLRAIAAAALLTREASKLTFGQDIEITSSHNLESLLRSPPDKWLTNARITQYQV
LLLDPPRVRFKQTAALNPATLLPETDDTLPIHHCLDTLDSLTSTRPDLTDQPLAQAEA
TLFTDGSSYIRDGKRYAGAAVVTLDSVIWAEPLPIGTSAQKAELIALTKALEWSKDK
SVNIYTDSRYAFATLHVHGMIYRERGWLTAGGKAIKNAPEILALLTAVWLPKRVAV
MHCKGHQKDDAPTSTGNRRADEVAREVAIRPLSTQATISDAPDMPDTETPQYSNVE
EALG

In some embodiments, the present disclosure provides reverse transcriptases, and prime editors (e.g. fusion proteins or prime editors in which each component is provided in trans) comprising reverse transcriptases, wherein the reverse transcriptase is a KORV reverse transcriptase of SEQ ID NO: 50, or a KORV reverse transcriptase variant having at least 70%, at least 75%, at least 80%, at least 85%, at least 90%, at least 95%, at least 96%, at least 97%, at least 98%, or at least 99% sequence identity with SEQ ID NO: 50, wherein the KORV reverse transcriptase variant comprises one or more mutations selected from the group consisting of D197N, T303K, W310F, E327P, and L599W. In some embodiments, the KORV reverse transcriptase variant comprises two or more, three or more, four or more, or all five of these mutations. In some embodiments, the KORV reverse transcriptase variant comprises the mutation D197N. In some embodiments, the KORV reverse transcriptase variant comprises the mutation T303K. In some embodiments, the KORV reverse transcriptase variant comprises the mutation W310F. In some embodiments, the KORV reverse transcriptase variant comprises the mutation E327P. In some embodiments, the KORV reverse transcriptase variant comprises the mutation L599W.

In certain embodiments, the KORV reverse transcriptase variant comprises the amino acid sequence of any one of SEQ ID NOs: 220-225, or an amino acid sequence at least 70%, at least 75%, at least 80%, at least 85%, at least 90%, at least 95%, at least 96%, at least 97%, at least 98%, or at least 99% identical to any one of SEQ ID NOs: 220-225, wherein the amino acid sequence comprises at least one of the residues 197N, 303K, 310F, 327P, and 599W:

KORV-RT D197N:
(SEQ ID NO: 220)
MNLEEEYRLHEKPVPPSIDPSWLQLFPMVWAEKAGMGLANQVPPVVVELKSDASPV
AVRQYPMSKEAREGIRPHIQRFLDLGILVPCQSPWNTPLLPVKKPGTNDYRPVQDLR
EVNKRVQDIHPTVPNPYNLLSSLPPSHTWYSVLDLKDAFFCLKLHPNSQPLFAFEWR
DPEKGNTGQLTWTRLPQGFKNSPTLFNEALHRDLASFRALNPQVVMLQYVDDLLVA
APTYRDCKEGTRRLLQELSKLGYRVSAKKAQLCREEVTYLGYLLKGGKRWLTPAR
KATVMKIPTPTTPRQVREFLGTAGFCRLWIPGFASLAAPLYPLTREKVPFTWTEAHQE
AFGRIKEALLSAPALALPDLTKPFALYVDEKEGVARGVLTQTLGPWRRPVAYLSKKL
DPVASGWPTCLKAIAAVALLLKDADKLTLGQNVLVIAPHNLESIVRQPPDRWMTNA
RMTHYQSLLLNERVSFAPPAILNPATLLPVESDDTPIHICSEILAEETGTRPDLRDQPLP
GVPAWYTDGSSFIMDGRRQAGAAIVDNKRTVWASNLPEGTSAQKAELIALTQALRL
AEGKSINIYTDSRYAFATAHVHGAIYKQRGLLTSAGKDIKNKEEILALLEAIHLPKRV
AIIHCPGHQRGTDPVATGNRKADEAAKQAAQSTRILTETTKNQEHFEPTRGK
KORV-RT T303K:
(SEQ ID NO: 221)
MNLEEEYRLHEKPVPPSIDPSWLQLFPMVWAEKAGMGLANQVPPVVVELKSDASPV
AVRQYPMSKEAREGIRPHIQRFLDLGILVPCQSPWNTPLLPVKKPGTNDYRPVQDLR
EVNKRVQDIHPTVPNPYNLLSSLPPSHTWYSVLDLKDAFFCLKLHPNSQPLFAFEWR
DPEKGNTGQLTWTRLPQGFKNSPTLFDEALHRDLASFRALNPQVVMLQYVDDLLVA
APTYRDCKEGTRRLLQELSKLGYRVSAKKAQLCREEVTYLGYLLKGGKRWLTPAR
KATVMKIPTPTTPRQVREFLGKAGFCRLWIPGFASLAAPLYPLTREKVPFTWTEAHQ
EAFGRIKEALLSAPALALPDLTKPFALYVDEKEGVARGVLTQTLGPWRRPVAYLSKK
LDPVASGWPTCLKAIAAVALLLKDADKLTLGQNVLVIAPHNLESIVRQPPDRWMTN
ARMTHYQSLLLNERVSFAPPAILNPATLLPVESDDTPIHICSEILAEETGTRPDLRDQPL
PGVPAWYTDGSSFIMDGRRQAGAAIVDNKRTVWASNLPEGTSAQKAELIALTQALR
LAEGKSINIYTDSRYAFATAHVHGAIYKQRGLLTSAGKDIKNKEEILALLEAIHLPKR
VAIIHCPGHQRGTDPVATGNRKADEAAKQAAQSTRILTETTKNQEHFEPTRGK
KORV-RT W310F:
(SEQ ID NO: 222)
MNLEEEYRLHEKPVPPSIDPSWLQLFPMVWAEKAGMGLANQVPPVVVELKSDASPV
AVRQYPMSKEAREGIRPHIQRFLDLGILVPCQSPWNTPLLPVKKPGTNDYRPVQDLR
EVNKRVQDIHPTVPNPYNLLSSLPPSHTWYSVLDLKDAFFCLKLHPNSQPLFAFEWR
DPEKGNTGQLTWTRLPQGFKNSPTLFDEALHRDLASFRALNPQVVMLQYVDDLLVA
APTYRDCKEGTRRLLQELSKLGYRVSAKKAQLCREEVTYLGYLLKGGKRWLTPAR
KATVMKIPTPTTPRQVREFLGTAGFCRLFIPGFASLAAPLYPLTREKVPFTWTEAHQE
AFGRIKEALLSAPALALPDLTKPFALYVDEKEGVARGVLTQTLGPWRRPVAYLSKKL
DPVASGWPTCLKAIAAVALLLKDADKLTLGQNVLVIAPHNLESIVRQPPDRWMTNA
RMTHYQSLLLNERVSFAPPAILNPATLLPVESDDTPIHICSEILAEETGTRPDLRDQPLP
GVPAWYTDGSSFIMDGRRQAGAAIVDNKRTVWASNLPEGTSAQKAELIALTQALRL
AEGKSINIYTDSRYAFATAHVHGAIYKQRGLLTSAGKDIKNKEEILALLEAIHLPKRV
AIIHCPGHQRGTDPVATGNRKADEAAKQAAQSTRILTETTKNQEHFEPTRGK
KORV-RT E327P:
(SEQ ID NO: 223)
MNLEEEYRLHEKPVPPSIDPSWLQLFPMVWAEKAGMGLANQVPPVVVELKSDASPV
AVRQYPMSKEAREGIRPHIQRFLDLGILVPCQSPWNTPLLPVKKPGTNDYRPVQDLR
EVNKRVQDIHPTVPNPYNLLSSLPPSHTWYSVLDLKDAFFCLKLHPNSQPLFAFEWR
DPEKGNTGQLTWTRLPQGFKNSPTLFDEALHRDLASFRALNPQVVMLQYVDDLLVA
APTYRDCKEGTRRLLQELSKLGYRVSAKKAQLCREEVTYLGYLLKGGKRWLTPAR
KATVMKIPTPTTPRQVREFLGTAGFCRLWIPGFASLAAPLYPLTRPKVPFTWTEAHQE
AFGRIKEALLSAPALALPDLTKPFALYVDEKEGVARGVLTQTLGPWRRPVAYLSKKL
DPVASGWPTCLKAIAAVALLLKDADKLTLGQNVLVIAPHNLESIVRQPPDRWMTNA
RMTHYQSLLLNERVSFAPPAILNPATLLPVESDDTPIHICSEILAEETGTRPDLRDQPLP
GVPAWYTDGSSFIMDGRRQAGAAIVDNKRTVWASNLPEGTSAQKAELIALTQALRL
AEGKSINIYTDSRYAFATAHVHGAIYKQRGLLTSAGKDIKNKEEILALLEAIHLPKRV
AIIHCPGHQRGTDPVATGNRKADEAAKQAAQSTRILTETTKNQEHFEPTRGK
KORV-RT L599W:
(SEQ ID NO: 224)
MNLEEEYRLHEKPVPPSIDPSWLQLFPMVWAEKAGMGLANQVPPVVVELKSDASPV
AVRQYPMSKEAREGIRPHIQRFLDLGILVPCQSPWNTPLLPVKKPGTNDYRPVQDLR
EVNKRVQDIHPTVPNPYNLLSSLPPSHTWYSVLDLKDAFFCLKLHPNSQPLFAFEWR
DPEKGNTGQLTWTRLPQGFKNSPTLFDEALHRDLASFRALNPQVVMLQYVDDLLVA
APTYRDCKEGTRRLLQELSKLGYRVSAKKAQLCREEVTYLGYLLKGGKRWLTPAR
KATVMKIPTPTTPRQVREFLGTAGFCRLWIPGFASLAAPLYPLTREKVPFTWTEAHQE
AFGRIKEALLSAPALALPDLTKPFALYVDEKEGVARGVLTQTLGPWRRPVAYLSKKL
DPVASGWPTCLKAIAAVALLLKDADKLTLGQNVLVIAPHNLESIVRQPPDRWMTNA
RMTHYQSLLLNERVSFAPPAILNPATLLPVESDDTPIHICSEILAEETGTRPDLRDQPLP
GVPAWYTDGSSFIMDGRRQAGAAIVDNKRTVWASNLPEGTSAQKAELIALTQALRL
AEGKSINIYTDSRYAFATAHVHGAIYKQRGWLTSAGKDIKNKEEILALLEAIHLPKRV
AIIHCPGHQRGTDPVATGNRKADEAAKQAAQSTRILTETTKNQEHFEPTRGK

In certain embodiments, the KORV reverse transcriptase variant comprises an amino acid sequence of SEQ ID NO: 225, or an amino acid sequence at least 70%, at least 75%, at least 80%, at least 85%, at least 90%, at least 95%, at least 96%, at least 97%, at least 98%, or at least 99% identical to SEQ ID NO: 225, wherein the amino acid sequence comprises the residues 197N, 303K, 310F, 327P, and 599W:

KORV_penta:
(SEQ ID NO: 225)
MNLEEEYRLHEKPVPPSIDPSWLQLFPMVWAEKAGMGLANQVPPVVVELKSDASPV
AVRQYPMSKEAREGIRPHIQRFLDLGILVPCQSPWNTPLLPVKKPGTNDYRPVQDLR
EVNKRVQDIHPTVPNPYNLLSSLPPSHTWYSVLDLKDAFFCLKLHPNSQPLFAFEWR
DPEKGNTGQLTWTRLPQGFKNSPTLFNEALHRDLASFRALNPQVVMLQYVDDLLVA
APTYRDCKEGTRRLLQELSKLGYRVSAKKAQLCREEVTYLGYLLKGGKRWLTPAR
KATVMKIPTPTTPRQVREFLGKAGFCRLFIPGFASLAAPLYPLTRPKVPFTWTEAHQE
AFGRIKEALLSAPALALPDLTKPFALYVDEKEGVARGVLTQTLGPWRRPVAYLSKKL
DPVASGWPTCLKAIAAVALLLKDADKLTLGQNVLVIAPHNLESIVRQPPDRWMTNA
RMTHYQSLLLNERVSFAPPAILNPATLLPVESDDTPIHICSEILAEETGTRPDLRDQPLP
GVPAWYTDGSSFIMDGRRQAGAAIVDNKRTVWASNLPEGTSAQKAELIALTQALRL
AEGKSINIYTDSRYAFATAHVHGAIYKQRGWLTSAGKDIKNKEEILALLEAIHLPKRV
AIIHCPGHQRGTDPVATGNRKADEAAKQAAQSTRILTETTKNQEHFEPTRGK

In some embodiments, the present disclosure provides reverse transcriptases, and prime editors (e.g. fusion proteins or prime editors in which each component is provided in trans) comprising reverse transcriptases, wherein the reverse transcriptase is a WMSV reverse transcriptase of SEQ ID NO: 54, or a WMSV reverse transcriptase variant having at least 70%, at least 75%, at least 80%, at least 85%, at least 90%, at least 95%, at least 96%, at least 97%, at least 98%, or at least 99% sequence identity with SEQ ID NO: 54, wherein the WMSV reverse transcriptase variant comprises one or more mutations selected from the group consisting of D197N, T303K, W311F, E327P, and L599W. In some embodiments, the WMSV reverse transcriptase variant comprises two or more, three or more, four or more, or all five of these mutations. In some embodiments, the WMSV reverse transcriptase variant comprises the mutation D197N. In some embodiments, the WMSV reverse transcriptase variant comprises the mutation T303K. In some embodiments, the WMSV reverse transcriptase variant comprises the mutation W311F. In some embodiments, the WMSV reverse transcriptase variant comprises the mutation E327P. In some embodiments, the WMSV reverse transcriptase variant comprises the mutation L599W.

In certain embodiments, the WMSV reverse transcriptase variant comprises the amino acid sequence of any one of SEQ ID NOs: 226-231, or an amino acid sequence at least 70%, at least 75%, at least 80%, at least 85%, at least 90%, at least 95%, at least 96%, at least 97%, at least 98%, or at least 99% identical to any one of SEQ ID NOs: 226-231, wherein the amino acid sequence comprises at least one of the residues 197N, 303K, 311F, 327P, and 599W:

WMSV-RT D197N:
(SEQ ID NO: 226)
LNLEEEYRLHEKPVPSSIDPSWLQLFPTVWAERAGMGLANQVPPVVVELRSGASPVA
VRQYPMSKEAREGIRPHIQRFLDLGVLVPCQSPWNTPLLPVKKPGTNDYRPVQDLRE
INKRVQDIHPTVPNPYNLLSSLPPSHTWYSVLDLKDAFFCLKLHPNSQPLFAFEWRDP
EKGNTGQLTWTRLPQGFKNSPTLFNEALHRDLAPFRALNPQVVLLQYVDDLLVAAP
TYRDCKEGTQKLLQELSKLGYRVSAKKAQLCQKEVTYLGYLLKEGKRWLTPARKA
TVMKIPPPTTPRQVREFLGTAGFCRLWIPGFASLAAPLYPLTKESIPFIWTEEHQKAFD
RIKEALLSAPALALPDLTKPFTLYVDERAGVARGVLTQTLGPWRRPVAYLSKKLDPV
ASGWPTCLKAVAAVALLLKDADKLTLGQNVTVIASHSLESIVRQPPDRWMTNARM
THYQSLLLNERVSFAPPAVLNPATLLPVESEATPVHRCSEILAEETGTRRDLKDQPLP
GVPAWYTDGSSFIAEGKRRAGAAIVDGKRTVWASSLPEGTSAQKAELVALTQALRL
AEGKDINIYTDSRYAFATAHIHGAIYKQRGLLTSAGKDIKNKEEILALLEAIHLPKRVA
IIHCPGHQKGNDPVATGNRRADEAAKQAALSTRVLAETTKPQELI
WMSV-RT T303K:
(SEQ ID NO: 227)
LNLEEEYRLHEKPVPSSIDPSWLQLFPTVWAERAGMGLANQVPPVVVELRSGASPVA
VRQYPMSKEAREGIRPHIQRFLDLGVLVPCQSPWNTPLLPVKKPGTNDYRPVQDLRE
INKRVQDIHPTVPNPYNLLSSLPPSHTWYSVLDLKDAFFCLKLHPNSQPLFAFEWRDP
EKGNTGQLTWTRLPQGFKNSPTLFDEALHRDLAPFRALNPQVVLLQYVDDLLVAAP
TYRDCKEGTQKLLQELSKLGYRVSAKKAQLCQKEVTYLGYLLKEGKRWLTPARKA
TVMKIPPPTTPRQVREFLGKAGFCRLWIPGFASLAAPLYPLTKESIPFIWTEEHQKAFD
RIKEALLSAPALALPDLTKPFTLYVDERAGVARGVLTQTLGPWRRPVAYLSKKLDPV
ASGWPTCLKAVAAVALLLKDADKLTLGQNVTVIASHSLESIVRQPPDRWMTNARM
THYQSLLLNERVSFAPPAVLNPATLLPVESEATPVHRCSEILAEETGTRRDLKDQPLP
GVPAWYTDGSSFIAEGKRRAGAAIVDGKRTVWASSLPEGTSAQKAELVALTQALRL
AEGKDINIYTDSRYAFATAHIHGAIYKQRGLLTSAGKDIKNKEEILALLEAIHLPKRVA
IIHCPGHQKGNDPVATGNRRADEAAKQAALSTRVLAETTKPQELI
WMSV-RT W311F:
(SEQ ID NO: 228)
LNLEEEYRLHEKPVPSSIDPSWLQLFPTVWAERAGMGLANQVPPVVVELRSGASPVA
VRQYPMSKEAREGIRPHIQRFLDLGVLVPCQSPWNTPLLPVKKPGTNDYRPVQDLRE
INKRVQDIHPTVPNPYNLLSSLPPSHTWYSVLDLKDAFFCLKLHPNSQPLFAFEWRDP
EKGNTGQLTWTRLPQGFKNSPTLFDEALHRDLAPFRALNPQVVLLQYVDDLLVAAP
TYRDCKEGTQKLLQELSKLGYRVSAKKAQLCQKEVTYLGYLLKEGKRWLTPARKA
TVMKIPPPTTPRQVREFLGTAGFCRLFIPGFASLAAPLYPLTKESIPFIWTEEHQKAFDR
IKEALLSAPALALPDLTKPFTLYVDERAGVARGVLTQTLGPWRRPVAYLSKKLDPVA
SGWPTCLKAVAAVALLLKDADKLTLGQNVTVIASHSLESIVRQPPDRWMTNARMT
HYQSLLLNERVSFAPPAVLNPATLLPVESEATPVHRCSEILAEETGTRRDLKDQPLPG
VPAWYTDGSSFIAEGKRRAGAAIVDGKRTVWASSLPEGTSAQKAELVALTQALRLA
EGKDINIYTDSRYAFATAHIHGAIYKQRGLLTSAGKDIKNKEEILALLEAIHLPKRVAII
HCPGHQKGNDPVATGNRRADEAAKQAALSTRVLAETTKPQELI
WMSV-RT E327P:
(SEQ ID NO: 229)
LNLEEEYRLHEKPVPSSIDPSWLQLFPTVWAERAGMGLANQVPPVVVELRSGASPVA
VRQYPMSKEAREGIRPHIQRFLDLGVLVPCQSPWNTPLLPVKKPGTNDYRPVQDLRE
INKRVQDIHPTVPNPYNLLSSLPPSHTWYSVLDLKDAFFCLKLHPNSQPLFAFEWRDP
EKGNTGQLTWTRLPQGFKNSPTLFDEALHRDLAPFRALNPQVVLLQYVDDLLVAAP
TYRDCKEGTQKLLQELSKLGYRVSAKKAQLCQKEVTYLGYLLKEGKRWLTPARKA
TVMKIPPPTTPRQVREFLGTAGFCRLWIPGFASLAAPLYPLTKPSIPFIWTEEHQKAFD
RIKEALLSAPALALPDLTKPFTLYVDERAGVARGVLTQTLGPWRRPVAYLSKKLDPV
ASGWPTCLKAVAAVALLLKDADKLTLGQNVTVIASHSLESIVRQPPDRWMTNARM
THYQSLLLNERVSFAPPAVLNPATLLPVESEATPVHRCSEILAEETGTRRDLKDQPLP
GVPAWYTDGSSFIAEGKRRAGAAIVDGKRTVWASSLPEGTSAQKAELVALTQALRL
AEGKDINIYTDSRYAFATAHIHGAIYKQRGLLTSAGKDIKNKEEILALLEAIHLPKRVA
IIHCPGHQKGNDPVATGNRRADEAAKQAALSTRVLAETTKPQELI
WMSV-RT L599W:
(SEQ ID NO: 230)
LNLEEEYRLHEKPVPSSIDPSWLQLFPTVWAERAGMGLANQVPPVVVELRSGASPVA
VRQYPMSKEAREGIRPHIQRFLDLGVLVPCQSPWNTPLLPVKKPGTNDYRPVQDLRE
INKRVQDIHPTVPNPYNLLSSLPPSHTWYSVLDLKDAFFCLKLHPNSQPLFAFEWRDP
EKGNTGQLTWTRLPQGFKNSPTLFDEALHRDLAPFRALNPQVVLLQYVDDLLVAAP
TYRDCKEGTQKLLQELSKLGYRVSAKKAQLCQKEVTYLGYLLKEGKRWLTPARKA
TVMKIPPPTTPRQVREFLGTAGFCRLWIPGFASLAAPLYPLTKESIPFIWTEEHQKAFD
RIKEALLSAPALALPDLTKPFTLYVDERAGVARGVLTQTLGPWRRPVAYLSKKLDPV
ASGWPTCLKAVAAVALLLKDADKLTLGQNVTVIASHSLESIVRQPPDRWMTNARM
THYQSLLLNERVSFAPPAVLNPATLLPVESEATPVHRCSEILAEETGTRRDLKDQPLP
GVPAWYTDGSSFIAEGKRRAGAAIVDGKRTVWASSLPEGTSAQKAELVALTQALRL
AEGKDINIYTDSRYAFATAHIHGAIYKQRGWLTSAGKDIKNKEEILALLEAIHLPKRV
AIIHCPGHQKGNDPVATGNRRADEAAKQAALSTRVLAETTKPQELI

In certain embodiments, the WMSV reverse transcriptase variant comprises an amino acid sequence of SEQ ID NO: 231, or an amino acid sequence at least 70%, at least 75%, at least 80%, at least 85%, at least 90%, at least 95%, at least 96%, at least 97%, at least 98%, or at least 99% identical to SEQ ID NO: 231, wherein the amino acid sequence comprises the residues 197N, 303K, 311F, 327P, and 599W:

WMSV_penta:
(SEQ ID NO: 231)
LNLEEEYRLHEKPVPSSIDPSWLQLFPTVWAERAGMGLANQVPPVVVELRSGASPVA
VRQYPMSKEAREGIRPHIQRFLDLGVLVPCQSPWNTPLLPVKKPGTNDYRPVQDLRE
INKRVQDIHPTVPNPYNLLSSLPPSHTWYSVLDLKDAFFCLKLHPNSQPLFAFEWRDP
EKGNTGQLTWTRLPQGFKNSPTLFNEALHRDLAPFRALNPQVVLLQYVDDLLVAAP
TYRDCKEGTQKLLQELSKLGYRVSAKKAQLCQKEVTYLGYLLKEGKRWLTPARKA
TVMKIPPPTTPRQVREFLGKAGFCRLFIPGFASLAAPLYPLTKPSIPFIWTEEHQKAFD
RIKEALLSAPALALPDLTKPFTLYVDERAGVARGVLTQTLGPWRRPVAYLSKKLDPV
ASGWPTCLKAVAAVALLLKDADKLTLGQNVTVIASHSLESIVRQPPDRWMTNARM
THYQSLLLNERVSFAPPAVLNPATLLPVESEATPVHRCSEILAEETGTRRDLKDQPLP
GVPAWYTDGSSFIAEGKRRAGAAIVDGKRTVWASSLPEGTSAQKAELVALTQALRL
AEGKDINIYTDSRYAFATAHIHGAIYKQRGWLTSAGKDIKNKEEILALLEAIHLPKRV
AIIHCPGHQKGNDPVATGNRRADEAAKQAALSTRVLAETTKPQELI

In some embodiments, the reverse transcriptase comprises a PERV reverse transcriptase. For example, the prime editor proteins described herein may comprise a PERV reverse transcriptase comprising one or more mutations relative to the amino acid sequence of SEQ ID NO: 45. In some embodiments, the PERV reverse transcriptase comprises one or more mutations selected from the group consisting of D199N, T305K, W312F, E329P, and L602W relative to the amino acid sequence of SEQ ID NO: 45. In certain embodiments, the PERV reverse transcriptase comprises the mutations D199N, T305K, W312F, E329P, and L602W relative to the amino acid sequence of SEQ ID NO: 45. In some embodiments, the present disclosure provides reverse transcriptases, and prime editors (e.g. fusion proteins or prime editors in which each component is provided in trans) comprising reverse transcriptases, wherein the reverse transcriptase is a PERV reverse transcriptase of SEQ ID NO: 45, or a PERV reverse transcriptase variant having at least 70%, at least 75%, at least 80%, at least 85%, at least 90%, at least 95%, at least 96%, at least 97%, at least 98%, or at least 99% sequence identity with SEQ ID NO: 45, wherein the PERV reverse transcriptase variant comprises one or more mutations selected from the group consisting of D199N, T305K, W312F, E329P, and L602W. In some embodiments, the PERV reverse transcriptase variant comprises two or more, three or more, four or more, or all five of these mutations. In some embodiments, the PERV reverse transcriptase variant comprises the mutation D199N. In some embodiments, the PERV reverse transcriptase variant comprises the mutation T305K. In some embodiments, the PERV reverse transcriptase variant comprises the mutation W312F. In some embodiments, the PERV reverse transcriptase variant comprises the mutation E329P. In some embodiments, the PERV reverse transcriptase variant comprises the mutation L602W.

In certain embodiments, the PERV reverse transcriptase variant comprises the amino acid sequence of any one of SEQ ID NOs: 232-238, or an amino acid sequence at least 70%, at least 75%, at least 80%, at least 85%, at least 90%, at least 95%, at least 96%, at least 97%, at least 98%, or at least 99% identical to any one of SEQ ID NOs: 232-238, wherein the amino acid sequence comprises at least one of the residues 199N, 305K, 312F, 329P, and 602W:

PERV variant 21:
(SEQ ID NO: 232)
TLQLDDEYRLYSPQVKPDQDIQSWLEQFPQAWAETAGMGLAKQVPPQVIQLKASAT
PVSVRQYPLSREAREGIWPHVQRLIQQGILVPVQSPWNTPLLPVRKPGTNDYRPVQD
LREVNKRVQDIHPTVPNPYNLLSALPPERNWYTVLDLKDAFFCLRLHPTSQPLFAFE
WRDPGTGRTGQLTWTRLPQGFKNSPTIFDEALHRDLANFRIQHPQVTLLQYVDDLLL
AGATKQDCLEGTKALLLELSDLGYRASAKKAQICRREVTYLGYSLRGGQRWLTEAR
KKTVVQIPAPTTAKQVREFLGTAGFCRLWIPGFATLAAPLYPLTKEKGEFSWAPEHQ
KAFDAIKKALLSAPALALPDVTKPFTLYVDERKGVARGVLTQTLGPWRRPVAYLSK
KLDPVASGWPVCLKAIAAVAILVKDADKLTLGQNITVIAPHALENIVRQPPDRWMTN
ARMTHYQSLLLTERVTFAPPAALNPATLLPEETDEPVTHDCHQLLIEETGVRKDLTDI
PLTGEVLTWFTDGSSYVVEGKRMAGAAVVDGTHTIWASSLPEGTSAQKAELMALT
QALRLAEGKSINIYTDSRYAFATAHVHGAIYKQRGLLTSAGREIKNKEEILSLLEALH
LPKRLAIIHCPGHQKAKDLISRGNQMADRVAKQAAQAVNLLPI
PERV-RT D199N:
(SEQ ID NO: 233)
TLQLDDEYRLYSPQVKPDQDIQSWLEQFPQAWAETAGMGLAKQVPPQVIQLKASAT
PVSVRQYPLSREAREGIWPHVQRLIQQGILVPVQSPWNTPLLPVRKPGTNDYRPVQD
LREVNKRVQDIHPTVPNPYNLLSALPPERNWYTVLDLKDAFFCLRLHPTSQPLFAFE
WRDPGTGRTGQLTWTRLPQGFKNSPTIFNEALHRDLANFRIQHPQVTLLQYVDDLLL
AGATKQDCLEGTKALLLELSDLGYRASAKKAQICRREVTYLGYSLRGGQRWLTEAR
KKTVVQIPAPTTAKQVREFLGTAGFCRLWIPGFATLAAPLYPLTKEKGEFSWAPEHQ
KAFDAIKKALLSAPALALPDVTKPFTLYVDERKGVARGVLTQTLGPWRRPVAYLSK
KLDPVASGWPVCLKAIAAVAILVKDADKLTLGQNITVIAPHALENIVRQPPDRWMTN
ARMTHYQSLLLTERVTFAPPAALNPATLLPEETDEPVTHDCHQLLIEETGVRKDLTDI
PLTGEVLTWFTDGSSYVVEGKRMAGAAVVDGTHTIWASSLPEGTSAQKAELMALT
QALRLAEGKSINIYTDSRYAFATAHVHGAIYKQRGLLTSAGREIKNKEEILSLLEALH
LPKRLAIIHCPGHQKAKDLISRGNQMADRVAKQAAQAVNLLPI
PERV-RT T305K:
(SEQ ID NO: 234)
TLQLDDEYRLYSPQVKPDQDIQSWLEQFPQAWAETAGMGLAKQVPPQVIQLKASAT
PVSVRQYPLSREAREGIWPHVQRLIQQGILVPVQSPWNTPLLPVRKPGTNDYRPVQD
LREVNKRVQDIHPTVPNPYNLLSALPPERNWYTVLDLKDAFFCLRLHPTSQPLFAFE
WRDPGTGRTGQLTWTRLPQGFKNSPTIFDEALHRDLANFRIQHPQVTLLQYVDDLLL
AGATKQDCLEGTKALLLELSDLGYRASAKKAQICRREVTYLGYSLRGGQRWLTEAR
KKTVVQIPAPTTAKQVREFLGKAGFCRLWIPGFATLAAPLYPLTKEKGEFSWAPEHQ
KAFDAIKKALLSAPALALPDVTKPFTLYVDERKGVARGVLTQTLGPWRRPVAYLSK
KLDPVASGWPVCLKAIAAVAILVKDADKLTLGQNITVIAPHALENIVRQPPDRWMTN
ARMTHYQSLLLTERVTFAPPAALNPATLLPEETDEPVTHDCHQLLIEETGVRKDLTDI
PLTGEVLTWFTDGSSYVVEGKRMAGAAVVDGTHTIWASSLPEGTSAQKAELMALT
QALRLAEGKSINIYTDSRYAFATAHVHGAIYKQRGLLTSAGREIKNKEEILSLLEALH
LPKRLAIIHCPGHQKAKDLISRGNQMADRVAKQAAQAVNLLPI
PERV-RT W313F:
(SEQ ID NO: 235)
TLQLDDEYRLYSPQVKPDQDIQSWLEQFPQAWAETAGMGLAKQVPPQVIQLKASAT
PVSVRQYPLSREAREGIWPHVQRLIQQGILVPVQSPWNTPLLPVRKPGTNDYRPVQD
LREVNKRVQDIHPTVPNPYNLLSALPPERNWYTVLDLKDAFFCLRLHPTSQPLFAFE
WRDPGTGRTGQLTWTRLPQGFKNSPTIFDEALHRDLANFRIQHPQVTLLQYVDDLLL
AGATKQDCLEGTKALLLELSDLGYRASAKKAQICRREVTYLGYSLRGGQRWLTEAR
KKTVVQIPAPTTAKQVREFLGTAGFCRLFIPGFATLAAPLYPLTKEKGEFSWAPEHQK
AFDAIKKALLSAPALALPDVTKPFTLYVDERKGVARGVLTQTLGPWRRPVAYLSKK
LDPVASGWPVCLKAIAAVAILVKDADKLTLGQNITVIAPHALENIVRQPPDRWMTNA
RMTHYQSLLLTERVTFAPPAALNPATLLPEETDEPVTHDCHQLLIEETGVRKDLTDIP
LTGEVLTWFTDGSSYVVEGKRMAGAAVVDGTHTIWASSLPEGTSAQKAELMALTQ
ALRLAEGKSINIYTDSRYAFATAHVHGAIYKQRGLLTSAGREIKNKEEILSLLEALHLP
KRLAIIHCPGHQKAKDLISRGNQMADRVAKQAAQAVNLLPI
PERV-RT E329P:
(SEQ ID NO: 236)
TLQLDDEYRLYSPQVKPDQDIQSWLEQFPQAWAETAGMGLAKQVPPQVIQLKASAT
PVSVRQYPLSREAREGIWPHVQRLIQQGILVPVQSPWNTPLLPVRKPGTNDYRPVQD
LREVNKRVQDIHPTVPNPYNLLSALPPERNWYTVLDLKDAFFCLRLHPTSQPLFAFE
WRDPGTGRTGQLTWTRLPQGFKNSPTIFDEALHRDLANFRIQHPQVTLLQYVDDLLL
AGATKQDCLEGTKALLLELSDLGYRASAKKAQICRREVTYLGYSLRGGQRWLTEAR
KKTVVQIPAPTTAKQVREFLGTAGFCRLWIPGFATLAAPLYPLTKPKGEFSWAPEHQ
KAFDAIKKALLSAPALALPDVTKPFTLYVDERKGVARGVLTQTLGPWRRPVAYLSK
KLDPVASGWPVCLKAIAAVAILVKDADKLTLGQNITVIAPHALENIVRQPPDRWMTN
ARMTHYQSLLLTERVTFAPPAALNPATLLPEETDEPVTHDCHQLLIEETGVRKDLTDI
PLTGEVLTWFTDGSSYVVEGKRMAGAAVVDGTHTIWASSLPEGTSAQKAELMALT
QALRLAEGKSINIYTDSRYAFATAHVHGAIYKQRGLLTSAGREIKNKEEILSLLEALH
LPKRLAIIHCPGHQKAKDLISRGNQMADRVAKQAAQAVNLLPI
PERV-RT L602W:
(SEQ ID NO: 237)
TLQLDDEYRLYSPQVKPDQDIQSWLEQFPQAWAETAGMGLAKQVPPQVIQLKASAT
PVSVRQYPLSREAREGIWPHVQRLIQQGILVPVQSPWNTPLLPVRKPGTNDYRPVQD
LREVNKRVQDIHPTVPNPYNLLSALPPERNWYTVLDLKDAFFCLRLHPTSQPLFAFE
WRDPGTGRTGQLTWTRLPQGFKNSPTIFDEALHRDLANFRIQHPQVTLLQYVDDLLL
AGATKQDCLEGTKALLLELSDLGYRASAKKAQICRREVTYLGYSLRGGQRWLTEAR
KKTVVQIPAPTTAKQVREFLGTAGFCRLWIPGFATLAAPLYPLTKEKGEFSWAPEHQ
KAFDAIKKALLSAPALALPDVTKPFTLYVDERKGVARGVLTQTLGPWRRPVAYLSK
KLDPVASGWPVCLKAIAAVAILVKDADKLTLGQNITVIAPHALENIVRQPPDRWMTN
ARMTHYQSLLLTERVTFAPPAALNPATLLPEETDEPVTHDCHQLLIEETGVRKDLTDI
PLTGEVLTWFTDGSSYVVEGKRMAGAAVVDGTHTIWASSLPEGTSAQKAELMALT
QALRLAEGKSINIYTDSRYAFATAHVHGAIYKQRGWLTSAGREIKNKEEILSLLEALH
LPKRLAIIHCPGHQKAKDLISRGNQMADRVAKQAAQAVNLLPI

In certain embodiments, the PERV reverse transcriptase variant comprises an amino acid sequence of SEQ ID NO: 238, or an amino acid sequence at least 70%, at least 75%, at least 80%, at least 85%, at least 90%, at least 95%, at least 96%, at least 97%, at least 98%, or at least 99% identical to SEQ ID NO: 238, wherein the amino acid sequence comprises the residues 199N, 305K, 312F, 329P, and 602W:

PERV variant 21.6 (pentamutant comprising D199N,
T305K, W312F, E329P, and L602W substitutions):
(SEQ ID NO: 238)
TLQLDDEYRLYSPQVKPDQDIQSWLEQFPQAWAETAGMGLAKQVPPQVI
QLKASATPVSVRQYPLSREAREGIWPHVQRLIQQGILVPVQSPWNTPLL
PVRKPGTNDYRPVQDLREVNKRVQDIHPTVPNPYNLLSALPPERNWYTV
LDLKDAFFCLRLHPTSQPLFAFEWRDPGTGRTGQLTWTRLPQGFKNSPT
IFNEALHRDLANFRIQHPQVTLLQYVDDLLLAGATKQDCLEGTKALLLE
LSDLGYRASAKKAQICRREVTYLGYSLRGGQRWLTEARKKTVVQIPAPT
TAKQVREFLGKAGFCRLFIPGFATLAAPLYPLTKPKGEFSWAPEHQKAF
DAIKKALLSAPALALPDVTKPFTLYVDERKGVARGVLTQTLGPWRRPVA
YLSKKLDPVASGWPVCLKAIAAVAILVKDADKLTLGQNITVIAPHALEN
IVRQPPDRWMTNARMTHYQSLLLTERVTFAPPAALNPATLLPEETDEPV
THDCHQLLIEETGVRKDLTDIPLTGEVLTWFTDGSSYVVEGKRMAGAAV
VDGTHTIWASSLPEGTSAQKAELMALTQALRLAEGKSINIYTDSRYAFA
TAHVHGAIYKQRGWLTSAGREIKNKEEILSLLEALHLPKRLAIIHCPGH
QKAKDLISRGNQMADRVAKQAAQAVNLLPI

In some embodiments, the reverse transcriptase comprises a Tf1 reverse transcriptase. For example, the prime editor proteins described herein may comprise a Tf1 reverse transcriptase comprising one or more mutations relative to the amino acid sequence of SEQ ID NO: 55. In some embodiments, the Tf1 reverse transcriptase comprises one or more mutations selected from the group consisting of V14A, E22K, P70T, G72V, M102I, K106R, K118R, A139T, L158Q, F269L, S297Q, K356E, A363V, K413E, I423V, and S492N relative to the amino acid sequence of SEQ ID NO: 55. In certain embodiments, the Tf1 reverse transcriptase comprises any one of the following groups of amino acid substitutions relative to the amino acid sequence of SEQ ID NO: 55:
K118R and S297Q;
V14A, L158Q, F269L, and K356E;
K106R, L158Q, F269L, A363V, and I423V;
E22K, P70T, G72V, M102I, K106R, A139T, L158Q, F269L, A363V, K413E, and
S492N; or

P70T, G72V, M102I, K106R, L158Q, F269L, A363V, K413E, and S492N.

In some embodiments, the present disclosure provides reverse transcriptases, and prime editors (e.g. fusion proteins or prime editors in which each component is provided in trans) comprising reverse transcriptases, wherein the reverse transcriptase is a Tf1 reverse transcriptase of SEQ ID NO: 171, or a Tf1 reverse transcriptase variant having at least 70%, at least 75%, at least 80%, at least 85%, at least 90%, at least 95%, at least 96%, at least 97%, at least 98%, or at least 99% sequence identity with SEQ ID NO: 171, wherein the Tf1 reverse transcriptase variant comprises one or more mutations selected from the group consisting of V14A, E22K, I64L, I64W, P70T, G72V, M102I, K106R, K118R, L133N, A139T, L158Q, S188K, I260L, F269L, E274R, R288Q, Q293K, S297Q, N316Q, K321R, K356E, A363V, K413E, 1423V, and S492N relative to SEQ ID NO: 171. In some embodiments, the Tf1 reverse transcriptase variant comprises a single mutation, wherein the single mutation is an I64L mutation, an I64W mutation, a K118R mutation, an L133N mutation, an S188K mutation, an I260L mutation, an E274R mutation, an R288Q mutation, a Q293K mutation, an S297Q mutation, an N316Q mutation, or a K321R mutation.

In some embodiments, the Tf1 reverse transcriptase variant comprises any one of the following groups of mutations relative to the amino acid sequence of SEQ ID NO: 171: K118R and S297Q; V14A, L158Q, F269L, and K356E; E22K, P70T, G72V, M102I, K106R, A139T, L158Q, F269L, A363V, K413E, and S492N; P70T, G72V, M102I, K106R, L158Q, F269L, A363V, K413E, and S492N; K106R, L158Q, F269L, A363V, and I423V; K118R, S297Q, S188K, I64L, 1260L, and R288Q; E22K, P70T, G72V, M102I, K106R, A139T, L158Q, F269L, A363V, K413E, S492N, K118R, S297Q, S188K, 164L, and I260L; K118R and S188K; K118R, S188K, and I260L; K118R, S188K, I260L, and S297Q; or K118R, S188K, I260L, R288K, and S297Q.

In certain embodiments, the Tf1 reverse transcriptase variant comprises the amino acid sequence of any one of SEQ ID NOs: 196-213 and 241-245, or an amino acid sequence at least 70%, at least 75%, at least 80%, at least 85%, at least 90%, at least 95%, at least 96%, at least 97%, at least 98%, or at least 99% identical to any one of SEQ ID NOs: 196-213 and 241-245, wherein the amino acid sequence comprises at least one of residues 14A, 22K, 64L, 64W, 70T, 72V, 102I, 106R, 118R, 133N, 139T, 158Q, 188K, 260L, 269L, 274R, 288Q, 293K, 297Q, 316Q, 321R, 356E, 363V, 413E, 423V, 492N:

Tf1 variant 5.131:
(SEQ ID NO: 196)
ISSSKHTLSQMNKVSNIVKEPKLPDIYKEFKDITADTNTEKLPKPIKGLEFEVELTQEN
YRLPIRNYPLTPVKMQAMNDEINQGLKSGIIRESKAINACPVIFVPRKEGTLRMVVDY
RPLNKYVKPNIYPLPLIEQLLTKIQGSTIFTKLDLKSAYHQIRVRKGDEHKLAFRCPRG
VFEYLVMPYGISTAPAHFQYFINTILGEAKESHVVCYMDDILIHSKSESEHVKHVKDV
LQKLKNANLIINQAKCEFHQSQVKFIGYHISEKGLTPCQENIDKVLQWKQPKNRKEL
RQFLGQVNYLRKFIPKTSQLTHPLNKLLKKDVRWKWTPTQTQAIENIKQCLVSPPVL
RHFDFSKKILLETDVSDVAVGAVLSQKHDDDKYYPVGYYSAKMSKAQLNYSVSDK
EMLAIIKSLEHWRHYLESTIEPFKILTDHRNLIGRITNESEPENKRLARWQLFLQDENFEI
NYRPGSANHIADALSRIVDETEPIPKDNEDNSINFVNQISI
Tf1 variant 5.27:
(SEQ ID NO: 197)
ISSSKHTLSQMNKASNIVKEPELPDIYKEFKDITADTNTEKLPKPIKGLEFEVELTQEN
YRLPIRNYPLPPGKMQAMNDEINQGLKSGIIRESKAINACPVMFVPKKEGTLRMVVD
YKPLNKYVKPNIYPLPLIEQLLAKIQGSTIFTKLDLKSAYHQIRVRKGDEHKLAFRCPR
GVFEYLVMPYGISTAPAHFQYFINTILGEAKESHVVCYMDDILIHSKSESEHVKHVKD
VLQKLKNANLIINQAKCEFHQSQVKFIGYHISEKGLTPCQENIDKVLQWKQPKNRKE
LRQFLGSVNYLRKFIPKTSQLTHPLNKLLKKDVRWKWTPTQTQAIENIKQCLVSPPV
LRHFDFSKEILLETDASDVAVGAVLSQKHDDDKYYPVGYYSAKMSKAQLNYSVSD
KEMLAIIKSLKHWRHYLESTIEPFKILTDHRNLIGRITNESEPENKRLARWQLFLQDENFE
INYRPGSANHIADALSRIVDETEPIPKDSEDNSINFVNQISI
Tf1 variant 5.47:
(SEQ ID NO: 198)
ISSSKHTLSQMNKVSNIVKEPELPDIYKEFKDITADTNTEKLPKPIKGLEFEVELTQEN
YRLPIRNYPLPPGKMQAMNDEINQGLKSGIIRESKAINACPVMFVPRKEGTLRMVVD
YKPLNKYVKPNIYPLPLIEQLLAKIQGSTIFTKLDLKSAYHQIRVRKGDEHKLAFRCPR
GVFEYLVMPYGISTAPAHFQYFINTILGEAKESHVVCYMDDILIHSKSESEHVKHVKD
VLQKLKNANLIINQAKCEFHQSQVKFIGYHISEKGLTPCQENIDKVLQWKQPKNRKE
LRQFLGSVNYLRKFIPKTSQLTHPLNKLLKKDVRWKWTPTQTQAIENIKQCLVSPPV
LRHFDFSKKILLETDVSDVAVGAVLSQKHDDDKYYPVGYYSAKMSKAQLNYSVSD
KEMLAIIKSLKHWRHYLESTVEPFKILTDHRNLIGRITNESEPENKRLARWQLFLQDFNF
EINYRPGSANHIADALSRIVDETEPIPKDSEDNSINFVNQISI
Tf1 variant 5.59:
(SEQ ID NO: 199)
ISSSKHTLSQMNKVSNIVKEPKLPDIYKEFKDITADTNTEKLPKPIKGLEFEVELTQEN
YRLPIRNYPLTPVKMQAMNDEINQGLKSGIIRESKAINACPVIFVPRKEGTLRMVVDY
KPLNKYVKPNIYPLPLIEQLLTKIQGSTIFTKLDLKSAYHQIRVRKGDEHKLAFRCPRG
VFEYLVMPYGISTAPAHFQYFINTILGEAKESHVVCYMDDILIHSKSESEHVKHVKDV
LQKLKNANLIINQAKCEFHQSQVKFIGYHISEKGLTPCQENIDKVLQWKQPKNRKEL
RQFLGSVNYLRKFIPKTSQLTHPLNKLLKKDVRWKWTPTQTQAIENIKQCLVSPPVL
RHFDFSKKILLETDVSDVAVGAVLSQKHDDDKYYPVGYYSAKMSKAQLNYSVSDK
EMLAIIKSLEHWRHYLESTIEPFKILTDHRNLIGRITNESEPENKRLARWQLFLQDENFEI
NYRPGSANHIADALSRIVDETEPIPKDNEDNSINFVNQISI
Tf1 variant 5.60:
(SEQ ID NO: 200)
ISSSKHTLSQMNKVSNIVKEPELPDIYKEFKDITADTNTEKLPKPIKGLEFEVELTQEN
YRLPIRNYPLTPVKMQAMNDEINQGLKSGIIRESKAINACPVIFVPRKEGTLRMVVDY
KPLNKYVKPNIYPLPLIEQLLAKIQGSTIFTKLDLKSAYHQIRVRKGDEHKLAFRCPRG
VFEYLVMPYGISTAPAHFQYFINTILGEAKESHVVCYMDDILIHSKSESEHVKHVKDV
LQKLKNANLIINQAKCEFHQSQVKFIGYHISEKGLTPCQENIDKVLQWKQPKNRKEL
RQFLGSVNYLRKFIPKTSQLTHPLNKLLKKDVRWKWTPTQTQAIENIKQCLVSPPVL
RHFDFSKKILLETDVSDVAVGAVLSQKHDDDKYYPVGYYSAKMSKAQLNYSVSDK
EMLAIIKSLEHWRHYLESTIEPFKILTDHRNLIGRITNESEPENKRLARWQLFLQDENF
EINYRPGSANHIADALSRIVDETEPIPKDNEDNSINFVNQISISGGSKRTADGSEFEPKKKR
KV
Tf1 variant 5.612:
(SEQ ID NO: 201)
ISSSKHTLSQMNKVSNIVKEPKLPDIYKEFKDITADTNTEKLPKPIKGLEFEVELTQEN
YRLPLRNYPLTPVKMQAMNDEINQGLKSGIIRESKAINACPVIFVPRKEGTLRMVVD
YRPLNKYVKPNIYPLPLIEQLLTKIQGSTIFTKLDLKSAYHQIRVRKGDEHKLAFRCPR
GVFEYLVMPYGIKTAPAHFQYFINTILGEAKESHVVCYMDDILIHSKSESEHVKHVK
DVLQKLKNANLIINQAKCEFHQSQVKFLGYHISEKGLTPCQENIDKVLQWKQPKNRK
ELRQFLGQVNYLRKFIPKTSQLTHPLNKLLKKDVRWKWTPTQTQAIENIKQCLVSPP
VLRHFDFSKKILLETDVSDVAVGAVLSQKHDDDKYYPVGYYSAKMSKAQLNYSVS
DKEMLAIIKSLEHWRHYLESTIEPFKILTDHRNLIGRITNESEPENKRLARWQLFLQDF
NFEINYRPGSANHIADALSRIVDETEPIPKDNEDNSINFVNQISI
Tf1 variant 5.618:
(SEQ ID NO: 202)
ISSSKHTLSQMNKVSNIVKEPELPDIYKEFKDITADTNTEKLPKPIKGLEFEVELTQEN
YRLPIRNYPLPPGKMQAMNDEINQGLKSGIIRESKAINACPVMFVPKKEGTLRMVVD
YRPLNKYVKPNIYPLPLIEQLLAKIQGSTIFTKLDLKSAYHLIRVRKGDEHKLAFRCPR
GVFEYLVMPYGISTAPAHFQYFINTILGEAKESHVVCYMDDILIHSKSESEHVKHVKD
VLQKLKNANLIINQAKCEFHQSQVKFIGYHISEKGFTPCQENIDKVLQWKQPKNRKE
LRQFLGQVNYLRKFIPKTSQLTHPLNKLLKKDVRWKWTPTQTQAIENIKQCLVSPPV
LRHFDFSKKILLETDASDVAVGAVLSQKHDDDKYYPVGYYSAKMSKAQLNYSVSD
KEMLAIIKSLKHWRHYLESTIEPFKILTDHRNLIGRITNESEPENKRLARWQLFLQDENFEI
NYRPGSANHIADALSRIVDETEPIPKDSEDNSINFVNQISI
Tf1 variant S188K:
(SEQ ID NO: 203)
ISSSKHTLSQMNKVSNIVKEPELPDIYKEFKDITADTNTEKLPKPIKGLEFEVELTQEN
YRLPIRNYPLPPGKMQAMNDEINQGLKSGIIRESKAINACPVMFVPKKEGTLRMVVD
YKPLNKYVKPNIYPLPLIEQLLAKIQGSTIFTKLDLKSAYHLIRVRKGDEHKLAFRCPR
GVFEYLVMPYGIKTAPAHFQYFINTILGEAKESHVVCYMDDILIHSKSESEHVKHVK
DVLQKLKNANLIINQAKCEFHQSQVKFIGYHISEKGFTPCQENIDKVLQWKQPKNRK
ELRQFLGSVNYLRKFIPKTSQLTHPLNKLLKKDVRWKWTPTQTQAIENIKQCLVSPP
VLRHFDFSKKILLETDASDVAVGAVLSQKHDDDKYYPVGYYSAKMSKAQLNYSVS
DKEMLAIIKSLKHWRHYLESTIEPFKILTDHRNLIGRITNESEPENKRLARWOLFLQDFNFEI
NYRPGSANHIADALSRIVDETEPIPKDSEDNSINFVNQISI
Tf1 variant I260L:
(SEQ ID NO: 204)
ISSSKHTLSQMNKVSNIVKEPELPDIYKEFKDITADTNTEKLPKPIKGLEFEVELTQEN
YRLPIRNYPLPPGKMQAMNDEINQGLKSGIIRESKAINACPVMFVPKKEGTLRMVVD
YKPLNKYVKPNIYPLPLIEQLLAKIQGSTIFTKLDLKSAYHLIRVRKGDEHKLAFRCPR
GVFEYLVMPYGISTAPAHFQYFINTILGEAKESHVVCYMDDILIHSKSESEHVKHVKD
VLQKLKNANLIINQAKCEFHQSQVKFLGYHISEKGFTPCQENIDKVLQWKQPKNRKE
LRQFLGSVNYLRKFIPKTSQLTHPLNKLLKKDVRWKWTPTQTQAIENIKQCLVSPPV
LRHFDFSKKILLETDASDVAVGAVLSQKHDDDKYYPVGYYSAKMSKAQLNYSVSD
KEMLAIIKSLKHWRHYLESTIEPFKILTDHRNLIGRITNESEPENKRLARWQLFLQDEN
FEINYRPGSANHIADALSRIVDETEPIPKDSEDNSINFVNQISI
Tf1 variant R288Q:
(SEQ ID NO: 205)
ISSSKHTLSQMNKVSNIVKEPELPDIYKEFKDITADTNTEKLPKPIKGLEFEVELTQEN
YRLPIRNYPLPPGKMQAMNDEINQGLKSGIIRESKAINACPVMFVPKKEGTLRMVVD
YKPLNKYVKPNIYPLPLIEQLLAKIQGSTIFTKLDLKSAYHLIRVRKGDEHKLAFRCPR
GVFEYLVMPYGISTAPAHFQYFINTILGEAKESHVVCYMDDILIHSKSESEHVKHVKD
VLQKLKNANLIINQAKCEFHQSQVKFIGYHISEKGFTPCQENIDKVLQWKQPKNQKE
LRQFLGSVNYLRKFIPKTSQLTHPLNKLLKKDVRWKWTPTQTQAIENIKQCLVSPPV
LRHFDFSKKILLETDASDVAVGAVLSQKHDDDKYYPVGYYSAKMSKAQLNYSVSD
KEMLAIIKSLKHWRHYLESTIEPFKILTDHRNLIGRITNESEPENKRLARWQLFLQDENFE
INYRPGSANHIADALSRIVDETEPIPKDSEDNSINFVNQISI
Tf1 variant Q293K:
(SEQ ID NO: 206)
ISSSKHTLSQMNKVSNIVKEPELPDIYKEFKDITADTNTEKLPKPIKGLEFEVELTQEN
YRLPIRNYPLPPGKMQAMNDEINQGLKSGIIRESKAINACPVMFVPKKEGTLRMVVD
YKPLNKYVKPNIYPLPLIEQLLAKIQGSTIFTKLDLKSAYHLIRVRKGDEHKLAFRCPR
GVFEYLVMPYGISTAPAHFQYFINTILGEAKESHVVCYMDDILIHSKSESEHVKHVKD
VLQKLKNANLIINQAKCEFHQSQVKFIGYHISEKGFTPCQENIDKVLQWKQPKNRKE
LRKFLGSVNYLRKFIPKTSQLTHPLNKLLKKDVRWKWTPTQTQAIENIKQCLVSPPV
LRHFDFSKKILLETDASDVAVGAVLSQKHDDDKYYPVGYYSAKMSKAQLNYSVSD
KEMLAIIKSLKHWRHYLESTIEPFKILTDHRNLIGRITNESEPENKRLARWQLFLQDEN
FEINYRPGSANHIADALSRIVDETEPIPKDSEDNSINFVNQISI
Tf1 variant I64L:
(SEQ ID NO: 207)
ISSSKHTLSQMNKVSNIVKEPELPDIYKEFKDITADTNTEKLPKPIKGLEFEVELTQEN
YRLPLRNYPLPPGKMQAMNDEINQGLKSGIIRESKAINACPVMFVPKKEGTLRMVVD
YKPLNKYVKPNIYPLPLIEQLLAKIQGSTIFTKLDLKSAYHLIRVRKGDEHKLAFRCPR
GVFEYLVMPYGISTAPAHFQYFINTILGEAKESHVVCYMDDILIHSKSESEHVKHVKD
VLQKLKNANLIINQAKCEFHQSQVKFIGYHISEKGFTPCQENIDKVLQWKQPKNRKE
LRQFLGSVNYLRKFIPKTSQLTHPLNKLLKKDVRWKWTPTQTQAIENIKQCLVSPPV
LRHFDFSKKILLETDASDVAVGAVLSQKHDDDKYYPVGYYSAKMSKAQLNYSVSD
KEMLAIIKSLKHWRHYLESTIEPFKILTDHRNLIGRITNESEPENKRLARWQLFLQDENFE
INYRPGSANHIADALSRIVDETEPIPKDSEDNSINFVNQISI
Tf1 variant I64W:
(SEQ ID NO: 208)
ISSSKHTLSQMNKVSNIVKEPELPDIYKEFKDITADTNTEKLPKPIKGLEFEVELTQEN
YRLPWRNYPLPPGKMQAMNDEINQGLKSGIIRESKAINACPVMFVPKKEGTLRMVV
DYKPLNKYVKPNIYPLPLIEQLLAKIQGSTIFTKLDLKSAYHLIRVRKGDEHKLAFRCP
RGVFEYLVMPYGISTAPAHFQYFINTILGEAKESHVVCYMDDILIHSKSESEHVKHVK
DVLQKLKNANLIINQAKCEFHQSQVKFIGYHISEKGFTPCQENIDKVLQWKQPKNRK
ELRQFLGSVNYLRKFIPKTSQLTHPLNKLLKKDVRWKWTPTQTQAIENIKQCLVSPP
VLRHFDFSKKILLETDASDVAVGAVLSQKHDDDKYYPVGYYSAKMSKAQLNYSVS
DKEMLAIIKSLKHWRHYLESTIEPFKILTDHRNLIGRITNESEPENKRLARWQLFLQDFNFE
INYRPGSANHIADALSRIVDETEPIPKDSEDNSINFVNQISI
Tf1 variant N316Q:
(SEQ ID NO: 209)
ISSSKHTLSQMNKVSNIVKEPELPDIYKEFKDITADTNTEKLPKPIKGLEFEVELTQEN
YRLPIRNYPLPPGKMQAMNDEINQGLKSGIIRESKAINACPVMFVPKKEGTLRMVVD
YKPLNKYVKPNIYPLPLIEQLLAKIQGSTIFTKLDLKSAYHLIRVRKGDEHKLAFRCPR
GVFEYLVMPYGISTAPAHFQYFINTILGEAKESHVVCYMDDILIHSKSESEHVKHVKD
VLQKLKNANLIINQAKCEFHQSQVKFIGYHISEKGFTPCQENIDKVLQWKQPKNRKE
LRQFLGSVNYLRKFIPKTSQLTHPLQKLLKKDVRWKWTPTQTQAIENIKQCLVSPPV
LRHFDFSKKILLETDASDVAVGAVLSQKHDDDKYYPVGYYSAKMSKAQLNYSVSD
KEMLAIIKSLKHWRHYLESTIEPFKILTDHRNLIGRITNESEPENKRLARWQLFLQDEN
FEINYRPGSANHIADALSRIVDETEPIPKDSEDNSINFVNQISI
Tf1 variant K321R:
(SEQ ID NO: 210)
ISSSKHTLSQMNKVSNIVKEPELPDIYKEFKDITADTNTEKLPKPIKGLEFEVELTQEN
YRLPIRNYPLPPGKMQAMNDEINQGLKSGIIRESKAINACPVMFVPKKEGTLRMVVD
YKPLNKYVKPNIYPLPLIEQLLAKIQGSTIFTKLDLKSAYHLIRVRKGDEHKLAFRCPR
GVFEYLVMPYGISTAPAHFQYFINTILGEAKESHVVCYMDDILIHSKSESEHVKHVKD
VLQKLKNANLIINQAKCEFHQSQVKFIGYHISEKGFTPCQENIDKVLQWKQPKNRKE
LRQFLGSVNYLRKFIPKTSQLTHPLNKLLKRDVRWKWTPTQTQAIENIKQCLVSPPVL
RHFDFSKKILLETDASDVAVGAVLSQKHDDDKYYPVGYYSAKMSKAQLNYSVSDK
EMLAIIKSLKHWRHYLESTIEPFKILTDHRNLIGRITNESEPENKRLARWQLFLQDFNFEINY
RPGSANHIADALSRIVDETEPIPKDSEDNSINFVNQISI
Tf1 variant L133N:
(SEQ ID NO: 211)
ISSSKHTLSQMNKVSNIVKEPELPDIYKEFKDITADTNTEKLPKPIKGLEFEVELTQEN
YRLPIRNYPLPPGKMQAMNDEINQGLKSGIIRESKAINACPVMFVPKKEGTLRMVVD
YKPLNKYVKPNIYPLPNIEQLLAKIQGSTIFTKLDLKSAYHLIRVRKGDEHKLAFRCPR
GVFEYLVMPYGISTAPAHFQYFINTILGEAKESHVVCYMDDILIHSKSESEHVKHVKD
VLQKLKNANLIINQAKCEFHQSQVKFIGYHISEKGFTPCQENIDKVLQWKQPKNRKE
LRQFLGSVNYLRKFIPKTSQLTHPLNKLLKKDVRWKWTPTQTQAIENIKQCLVSPPV
LRHFDFSKKILLETDASDVAVGAVLSQKHDDDKYYPVGYYSAKMSKAQLNYSVSD
KEMLAIIKSLKHWRHYLESTIEPFKILTDHRNLIGRITNESEPENKRLARWQLFLQDENFEI
NYRPGSANHIADALSRIVDETEPIPKDSEDNSINFVNQISI
Tf1 variant K118R:
(SEQ ID NO: 212)
ISSSKHTLSQMNKVSNIVKEPELPDIYKEFKDITADTNTEKLPKPIKGLEFEVELTQEN
YRLPIRNYPLPPGKMQAMNDEINQGLKSGIIRESKAINACPVMFVPKKEGTLRMVVD
YRPLNKYVKPNIYPLPLIEQLLAKIQGSTIFTKLDLKSAYHLIRVRKGDEHKLAFRCPR
GVFEYLVMPYGISTAPAHFQYFINTILGEAKESHVVCYMDDILIHSKSESEHVKHVKD
VLQKLKNANLIINQAKCEFHQSQVKFIGYHISEKGFTPCQENIDKVLQWKQPKNRKE
LRQFLGSVNYLRKFIPKTSQLTHPLNKLLKKDVRWKWTPTQTQAIENIKQCLVSPPV
LRHFDFSKKILLETDASDVAVGAVLSQKHDDDKYYPVGYYSAKMSKAQLNYSVSD
KEMLAIIKSLKHWRHYLESTIEPFKILTDHRNLIGRITNESEPENKRLARWQLFLQDENFEI
NYRPGSANHIADALSRIVDETEPIPKDSEDNSINFVNQISI
Tf1 variant K118R: Tf1 variant S297Q:
(SEQ ID NO: 213)
ISSSKHTLSQMNKVSNIVKEPELPDIYKEFKDITADTNTEKLPKPIKGLEFEVELTQEN
YRLPIRNYPLPPGKMQAMNDEINQGLKSGIIRESKAINACPVMFVPKKEGTLRMVVD
YKPLNKYVKPNIYPLPLIEQLLAKIQGSTIFTKLDLKSAYHLIRVRKGDEHKLAFRCPR
GVFEYLVMPYGISTAPAHFQYFINTILGEAKESHVVCYMDDILIHSKSESEHVKHVKD
VLQKLKNANLIINQAKCEFHQSQVKFIGYHISEKGFTPCQENIDKVLQWKQPKNRKE
LRQFLGQVNYLRKFIPKTSQLTHPLNKLLKKDVRWKWTPTQTQAIENIKQCLVSPPV
LRHFDFSKKILLETDASDVAVGAVLSQKHDDDKYYPVGYYSAKMSKAQLNYSVSD
KEMLAIIKSLKHWRHYLESTIEPFKILTDHRNLIGRITNESEPENKRLARWQLFLQDENFEI
NYRPGSANHIADALSRIVDETEPIPKDSEDNSINFVNQISI
Tf1-rat4:
(SEQ ID NO: 241)
MISSSKHTLSQMNKVSNIVKEPELPDIYKEFKDITADTNTEKLPKPIKGLEFEVELTQE
NYRLPIRNYPLPPGKMQAMNDEINQGLKSGIIRESKAINACPVMFVPKKEGTLRMVV
DYRPLNKYVKPNIYPLPLIEQLLAKIQGSTIFTKLDLKSAYHLIRVRKGDEHKLAFRCP
RGVFEYLVMPYGIKTAPAHFQYFINTILGEAKESHVVCYMDDILIHSKSESEHVKHVK
DVLQKLKNANLIINQAKCEFHQSQVKFLGYHISEKGFTPCQENIDKVLQWKQPKNQK
ELRQFLGQVNYLRKFIPKTSQLTHPLNKLLKKDVRWKWTPTQTQAIENIKQCLVSPP
VLRHFDFSKKILLETDASDVAVGAVLSQKHDDDKYYPVGYYSAKMSKAQLNYSVS
DKEMLAIIKSLKHWRHYLESTIEPFKILTDHRNLIGRITNESEPENKRLARWQLFLQDF
NFEINYRPGSANHIADALSRIVDETEPIPKDSEDNSINFVNQISI
Tf1evo3.1:
(SEQ ID NO: 242)
ISSSKHTLSQMNKVSNIVKEPELPDIYKEFKDITADTNTEKLPKPIKGLEFEVELTQEN
YRLPIRNYPLTPVKMQAMNDEINQGLKSGIIRESKAINACPVIFVPRKEGTLRMVVDY
KPLNKYVKPNIYPLPLIEQLLAKIQGSTIFTKLDLKSAYHQIRVRKGDEHKLAFRCPRG
VFEYLVMPYGISTAPAHFQYCINTILGEAKESHVVCYMDDILIHSKSESEHVKHVKDV
LQKLKNANLIINQAKCEFHQSQVKFIGYHISEKGLTPCQENIDKVLQWKQPKNRKEL
RQFLGSVNYLRKFIPKTSQLTHPLNKLLKKDVRWKWTPTQTQAIENIKQCLVSPPVL
RHFDFSKKILLETDVSDVAVGAVLSQKHDDDKYYPVGYYSAKMSKAQLNYSVSDK
EMLAIIKSLEHWRHYLESTIEPFKILTDHRNLIGRITNESEPENKRLARWQLFLQDENF
EINYRPGSANHIADALSRIVDETEPIPKDNEDNSINFVNQISI
Tf1evo3.2:
(SEQ ID NO: 243)
ISSSKHTLSQMNKVSNIVKEPELPDIYKEFKDITADTNTEKLPKPIKGLEFEVELTQEN
YRLPIRNYPLTPVKMQAMNDEINQGLKGGIIRESKAINACPVIFVPRKEGTLRMVVDY
RPLNKYVKPNVYPLPLIEQLLAKIQGSTIFTKLDLKSAYHQIRVRKGDEHKLAFRCPR
GVFEYLVMPYGISTAPAHFQYFINTILGEAKESHVVCYMDDILIHSKSESEHVKHVKD
VLQKLKNANLIINQAKCEFHQSQVKFIGYHISEKGLTPCQENIDKVLQWKQPKNRKE
LRQFLGSVNYLRKFIPKTSQLTHPLNKLLKKDVRWKWTPTQTQAIENIKQCLVSPPV
LRHFDFSKKILLETDVSDVAVGAVLSQKHDDDKYYPVGYYSAKMSKAQLNYSVSD
KEMLAIIKSLEHWRHYLESTIEPFKILTDHRNLIGRITNESEPENKRLARWQLFLQDENFEIN
YRPGSANHIADALSRIVDETEPIPKDNEDNSINFVNQISI
Tf1evo + rat-1:
(SEQ ID NO: 244)
ISSSKHTLSQMNKVSNIVKEPELPDIYKEFKDITADTNTEKLPKPIKGLEFEVELTQEN
YRLPIRNYPLTPVKMQAMNDEINQGLKGGIIRESKAINACPVIFVPRKEGTLRMVVDY
RPLNKYVKPNVYPLPLIEQLLAKIQGSTIFTKLDLKSAYHQIRVRKGDEHKLAFRCPR
GVFEYLVMPYGIKTAPAHFQYFINTILGEAKESHVVCYMDDILIHSKSESEHVKHVK
DVLQKLKNANLIINQAKCEFHQSQVKFLGYHISEKGLTPCQENIDKVLQWKQPKNQ
KELRQFLGQVNYLRKFIPKTSQLTHPLNKLLKKDVRWKWTPTQTQAIENIKQCLVSP
PVLRHFDFSKKILLETDVSDVAVGAVLSQKHDDDKYYPVGYYSAKMSKAQLNYSVS
DKEMLAIIKSLEHWRHYLESTIEPFKILTDHRNLIGRITNESEPENKRLARWQLFLQDFNFE
INYRPGSANHIADALSRIVDETEPIPKDNEDNSINFVNQISI
Tf1evo + rat2:
(SEQ ID NO: 245)
ISSSKHTLSQMNKVSNIVKEPELPDIYKEFKDITADTNTEKLPKPIKGLEFEVELTQEN
YRLPIRNYPLTPVKMQAMNDEINQGLKSGIIRESKAINACPVIFVPRKEGTLRMVVDY
RPLNKYVKPNIYPLPLIEQLLAKIQGSTIFTKLDLKSAYHQIRVRKGDEHKLAFRCPRG
VFEYLVMPYGIKTAPAHFQYCINTILGEAKESHVVCYMDDILIHSKSESEHVKHVKD
VLQKLKNANLIINQAKCEFHQSQVKFLGYHISEKGLTPCQENIDKVLQWKQPKNQKE
LRQFLGQVNYLRKFIPKTSQLTHPLNKLLKKDVRWKWTPTQTQAIENIKQCLVSPPV
LRHFDFSKKILLETDVSDVAVGAVLSQKHDDDKYYPVGYYSAKMSKAQLNYSVSD
KEMLAIIKSLEHWRHYLESTIEPFKILTDHRNLIGRITNESEPENKRLARWQLFLQDENFEIN
YRPGSANHIADALSRIVDETEPIPKDNEDNSINFVNQISI

In some embodiments, the reverse transcriptase comprises an Ec48 reverse transcriptase. For example, the prime editor proteins described herein may comprise an Ec48 reverse transcriptase comprising one or more mutations relative to the amino acid sequence of SEQ ID NO: 59. In some embodiments, the Ec48 reverse transcriptase comprises one or more mutations selected from the group consisting of A36V, E54K, K87E, R205K, V214L, D243N, R267I, S277F, E279K, N317S, K318E, H324Q, K326E, E328K, and R372K relative to the amino acid sequence of SEQ ID NO: 59. In certain embodiments, the Ec48 reverse transcriptase comprises any one of the following groups of amino acid substitutions relative to the amino acid sequence of SEQ ID NO: 59:
R267I, K318E, K326E, E328K, and R372K;
K87E, R205K, V214L, D243N, R267I, N317S, K318E, H324Q, and K326E;
E54K, K87E, D243N, R267I, E279K, and K318E;
A36V, K87E, R205K, D243N, R267I, E279K, and K318E;
E54K, K87E, D243N, R267I, E279K, and K318E; or
E54K, K87E, D243N, R267I, S277F, E279K, and K318E.

In some embodiments, the present disclosure provides reverse transcriptases, and prime editors (e.g. fusion proteins or prime editors in which each component is provided in trans) comprising reverse transcriptases, wherein the reverse transcriptase is an Ec48 reverse transcriptase of SEQ ID NO: 59, or an Ec48 reverse transcriptase variant having at least 70%, at least 75%, at least 80%, at least 85%, at least 90%, at least 95%, at least 96%, at least 97%, at least 98%, or at least 99% sequence identity with SEQ ID NO: 59, wherein the Ec48 reverse transcriptase variant comprises one or more mutations selected from the group consisting of A36V, E54K, E60K, K87E, S151T, E165D, L182N, T189N, R205K, V214L, D243N, R267I, S277F, E279K, V303M, K307R, R315K, N317S, K318E, H324Q, K326E, E328K, K343N, R372K, R378K, and T385R relative to SEQ ID NO: 59. In some embodiments, the Ec48 reverse transcriptase variant comprises a single mutation, wherein the single mutation is an L182N mutation, a T189N mutation, a K307R mutation, an R315K mutation, an R378K mutation, or a T385R mutation.

In some embodiments, the Ec48 reverse transcriptase variant comprises any one of the following groups of mutations relative to the amino acid sequence of SEQ ID NO: 59: R267I, K318E, K326E, E328K, and R372K; K87E, R205K, V214L, D243N, R267I, N317S, K318E, H324Q, and K326E; E54K, K87E, D243N, R267I, E279K, and K318E; A36V, K87E, R205K, D243N, R267I, E279K, and K318E; E54K, K87E, D243N, R267I, E279K, and K318E; E54K, K87E, D243N, R267I, S277F, E279K, and K318E; E60K, K87E, E165D, D243N, R267I, E279K, K318E, and K343N; E60K, K87E, S151T, E165D, D243N, R267I, E279K, V303M, K318E, and K343N; or R315K, L182N, and T189N.

In certain embodiments, the Ec48 reverse transcriptase variant comprises the amino acid sequence of any one of SEQ ID NOs: 188-195 or an amino acid sequence at least 70%, at least 75%, at least 80%, at least 85%, at least 90%, at least 95%, at least 96%, at least 97%, at least 98%, or at least 99% identical to any one of SEQ ID NOs: 188-195, wherein the amino acid sequence comprises at least one of residues 36V, 54K, 60K, 87E, 151T, 165D, 182N, 189N, 205K, 214L, 243N, 267I, 277F, 279K, 303M, 307R, 315K, 317S, 318E, 324Q, 326E, 328K, 343N, 372K, 378K, and 385R:

Ec48 variant 3.23:
(SEQ ID NO: 188)
GRPYVTLNLNGMFMDKFKPYSKSNAPITTLEKLSKALSISVEELKAIAELSLDEKYTL
KEIPKIDGSKRIVYSLHPKMRLLQSRINERIFKELVVFPSFLFGSVPSKNDVLNSNVKR
DYVSCAKAHCGAKTVLKVDISNFFDNIHRDLVRSVFEEILHIKDEALEYLVDICTKDD
FVVQGALTSSYIATLCLFAVEGDVVRRAQKKGLVYTRLLDDITVSSKISNYDFSQMQ
SHIERMLSEHNLPINKHKTKIFHCSSEPIKVHGLIVDYDSPRLPSDEVKRIRASIHNLKL
LAAKNNTKTSVAYRKEFNRCMGRVSELGRVGQEEYESFKKQLQAIKPMPSKRD
VAVIDAAIKSLELSYSKGNQNKHWYKRKYDLTRYKMIILTRSESFKEKLECFKSRLASLKPL
Ec48 variant 3.35 (or Ec48-evo2):
(SEQ ID NO: 189)
GRPYVTLNLNGMFMDKFKPYSKSNAPITTLEKLSKALSISVEELKAIAELSLDKKYTL
KEIPKIDGSKRIVYSLHPKMRLLQSRINERIFKELVVFPSFLFGSVPSKNDVLNSNVKR
DYVSCAKAHCGAKTVLKVDISNFFDNIHRDLVRSVFEEILHIKDEALEYLVDICTKDD
FVVQGALTSSYIATLCLFAVEGDVVRRAQRKGLVYTRLVDDITVSSKISNYDFSQMQ
SHIERMLSEHNLPINKHKTKIFHCSSEPIKVHGLIVDYDSPRLPSDKVKRIRASIHNLKL
LAAKNNTKTSVAYRKEFNRCMGRVNELGRVGHEKYESFKKQLQAIKPMPSKRDVA
VIDAAIKSLELSYSKGNQNKHWYKRKYDLTRYKMIILTRSESFKEKLECFKSRLASLKPL
Ec48 variant 3.36:
(SEQ ID NO: 190)
GRPYVTLNLNGMFMDKFKPYSKSNAPITTLEKLSKVLSISVEELKAIAELSLDEKYTL
KEIPKIDGSKRIVYSLHPKMRLLQSRINERIFKELVVFPSFLFGSVPSKNDVLNSNVKR
DYVSCAKAHCGAKTVLKVDISNFFDNIHRDLVRSVFEEILHIKDEALEYLVDICTKDD
FVVQGALTSSYIATLCLFAVEGDVVRRAQKKGLVYTRLVDDITVSSKISNYDFSQMQ
SHIERMLSEHNLPINKHKTKIFHCSSEPIKVHGLIVDYDSPRLPSDKVKRIRASIHNLKL
LAAKNNTKTSVAYRKEFNRCMGRVNELGRVGHEKYESFKKQLQAIKPMPSKRD
VAVIDAAIKSLELSYSKGNQNKHWYKRKYDLTRYKMIILTRSESFKEKLECFKSRLASLKPL
Ec48 variant 3.37:
(SEQ ID NO: 189)
GRPYVTLNLNGMFMDKFKPYSKSNAPITTLEKLSKALSISVEELKAIAELSLDKKYTL
KEIPKIDGSKRIVYSLHPKMRLLQSRINERIFKELVVFPSFLFGSVPSKNDVLNSNVKR
DYVSCAKAHCGAKTVLKVDISNFFDNIHRDLVRSVFEEILHIKDEALEYLVDICTKDD
FVVQGALTSSYIATLCLFAVEGDVVRRAQRKGLVYTRLVDDITVSSKISNYDFSQMQ
SHIERMLSEHNLPINKHKTKIFHCSSEPIKVHGLIVDYDSPRLPSDKVKRIRASIHNLKL
LAAKNNTKTSVAYRKEFNRCMGRVNELGRVGHEKYESFKKQLQAIKPMPSKRDVA
VIDAAIKSLELSYSKGNQNKHWYKRKYDLTRYKMIILTRSESFKEKLECFKSRLASLKPL
Ec48 variant 3.38:
(SEQ ID NO: 191)
GRPYVTLNLNGMFMDKFKPYSKSNAPITTLEKLSKALSISVEELKAIAELSLDKKYTL
KEIPKIDGSKRIVYSLHPKMRLLQSRINERIFKELVVFPSFLFGSVPSKNDVLNSNVKR
DYVSCAKAHCGAKTVLKVDISNFFDNIHRDLVRSVFEEILHIKDEALEYLVDICTKDD
FVVQGALTSSYIATLCLFAVEGDVVRRAQRKGLVYTRLVDDITVSSKISNYDFSQMQ
SHIERMLSEHNLPINKHKTKIFHCSSEPIKVHGLIVDYDSPRLPFDKVKRIRASIHNLKL
LAAKNNTKTSVAYRKEFNRCMGRVNELGRVGHEKYESFKKQLQAIKPMPSKRD
VAVIDAAIKSLELSYSKGNQNKHWYKRKYDLTRYKMIILTRSESFKEKLECFKSRLASLKPL
Ec48 variant 3.500:
(SEQ ID NO: 192)
GRPYVTLNLNGMFMDKFKPYSKSNAPITTLEKLSKALSISVEELKAIAELSLDEKYTL
KKIPKIDGSKRIVYSLHPKMRLLQSRINERIFKELVVFPSFLFGSVPSKNDVLNSNVKR
DYVSCAKAHCGAKTVLKVDISNFFDNIHRDLVRSVFEEILHIKDEALDYLVDICTKDD
FVVQGALTSSYIATLCLFAVEGDVVRRAQRKGLVYTRLVDDITVSSKISNYDFSQMQ
SHIERMLSEHNLPINKHKTKIFHCSSEPIKVHGLIVDYDSPRLPSDKVKRIRASIHNLKL
LAAKNNTKTSVAYRKEFNRCMGRVNELGRVGHEKYESFKKQLQAIKPMPSNRDVA
VIDAAIKSLELSYSKGNQNKHWYKRKYDLTRYKMIILTRSESFKEKLECFKSRLASLKPL
Ec48 variant 3.501:
(SEQ ID NO: 193)
GRPYVTLNLNGMFMDKFKPYSKSNAPITTLEKLSKALSISVEELKAIAELSLDEKYTL
KKIPKIDGSKRIVYSLHPKMRLLQSRINERIFKELVVFPSFLFGSVPSKNDVLNSNVKR
DYVSCAKAHCGAKTVLKVDISNFFDNIHRDLVRTVFEEILHIKDEALDYLVDICTKD
DFVVQGALTSSYIATLCLFAVEGDVVRRAQRKGLVYTRLVDDITVSSKISNYDFSQM
QSHIERMLSEHNLPINKHKTKIFHCSSEPIKVHGLIVDYDSPRLPSDKVKRIRASIHNLK
LLAAKNNTKTSMAYRKEFNRCMGRVNELGRVGHEKYESFKKQLQAIKPMPSNRD
VAVIDAAIKSLELSYSKGNQNKHWYKRKYDLTRYKMIILTRSESFKEKLECFKSRLASLKPL
Ec48 variant 3.8 (or Ec48-evo1):
(SEQ ID NO: 194)
GRPYVTLNLNGMFMDKFKPYSKSNAPITTLEKLSKALSISVEELKAIAELSLDEKYTL
KEIPKIDGSKRIVYSLHPKMRLLQSRINKRIFKELVVFPSFLFGSVPSKNDVLNSNVKR
DYVSCAKAHCGAKTVLKVDISNFFDNIHRDLVRSVFEEILHIKDEALEYLVDICTKDD
FVVQGALTSSYIATLCLFAVEGDVVRRAQRKGLVYTRLVDDITVSSKISNYDESQMQ
SHIERMLSEHDLPINKHKTKIFHCSSEPIKVHGLIVDYDSPRLPSDEVKRIRASIHNLKL
LAAKNNTKTSVAYRKEFNRCMGRVNELGRVGHEEYKSFKKQLQAIKPMPSKRDVA
VIDAAIKSLELSYSKGNQNKHWYKKKYDLTRYKMIILTRSESFKEKLECFKSRLASLKPL
Ec48-v2:
(SEQ ID NO: 195)
GRPYVTLNLNGMFMDKFKPYSKSNAPITTLEKLSKALSISVEELKAIAELSLDEKYTL
KEIPKIDGSKRIVYSLHPKMRLLQSRINKRIFKELVVFPSFLFGSVPSKNDVLNSNVKR
DYVSCAKAHCGAKTVLKVDISNFFDNIHRDLVRSVFEEILHIKDEALEYLVDICTKDD
FVVQGANTSSYIANLCLFAVEGDVVRRAQRKGLVYTRLVDDITVSSKISNYDFSQMQ
SHIERMLSEHDLPINKHKTKIFHCSSEPIKVHGLRVDYDSPRLPSDEVKRIRASIHNLK
LLAAKNNTKTSVAYRKEFNRCMGKVNKLGRVGHEKYESFKKQLQAIKPMPSKRDV
AVIDAAIKSLELSYSKGNQNKHWYKRKYDLTRYKMIILTRSESFKEKLECFKSRLASLKPL
Ec48-evo3:
(SEQ ID NO: 192)
GRPYVTLNLNGMFMDKFKPYSKSNAPITTLEKLSKALSISVEELKAIAELSLDEKYTL
KKIPKIDGSKRIVYSLHPKMRLLQSRINERIFKELVVFPSFLFGSVPSKNDVLNSNVKR
DYVSCAKAHCGAKTVLKVDISNFFDNIHRDLVRSVFEEILHIKDEALDYLVDICTKDD
FVVQGALTSSYIATLCLFAVEGDVVRRAQRKGLVYTRLVDDITVSSKISNYDFSQMQ
SHIERMLSEHNLPINKHKTKIFHCSSEPIKVHGLIVDYDSPRLPSDKVKRIRASIHNLKL
LAAKNNTKTSVAYRKEFNRCMGRVNELGRVGHEKYESFKKQLQAIKPMPSNRDVA
VIDAAIKSLELSYSKGNQNKHWYKRKYDLTRYKMIILTRSESFKEKLECFKSRLASLKPL

In some embodiments, the present disclosure provides reverse transcriptases, and prime editors (e.g. fusion proteins or prime editors in which each component is provided in trans) comprising reverse transcriptases, wherein the reverse transcriptase is an Ne144 reverse transcriptase of SEQ ID NO: 64, or an Ne144 reverse transcriptase variant having at least 70%, at least 75%, at least 80%, at least 85%, at least 90%, at least 95%, at least 96%, at least 97%, at least 98%, or at least 99% sequence identity with SEQ ID NO: 64, wherein the Ne144 reverse transcriptase variant comprises one or more mutations selected from the group consisting of A157T, A165T, and G288V relative to SEQ ID NO: 64. In some embodiments, the Ne144 reverse transcriptase variant comprises the mutations A157T, A165T, and G288V.

In certain embodiments, the Ne144 reverse transcriptase variant comprises the amino acid sequence of SEQ ID NO: 239, or an amino acid sequence at least 70%, at least 75%, at least 80%, at least 85%, at least 90%, at least 95%, at least 96%, at least 97%, at least 98%, or at least 99% identical to SEQ ID NO: 239, wherein the amino acid sequence comprises at least one of residues 157T, 165T, and 288V:

Ne144 RT 38.14:
(SEQ ID NO: 239)
AGQPTSREALYERIRSTSKEEVILEEMIRLGFWPAQGAVPHDPAEEIRR
RGELERQLSELREKSRKLYNEKALIAEQRKQRLAESRRKQKETKARRER
ERQERAQKWAQRKAGEILFLGEDVSGGMSHKTCDAELIKREGVPAIASA
EELARAMGITLKELRFLTYNRKVSRVTHYRRFLLPKKTGGLRLISAPMP
RLKRAQAWALEHIFNKLSFEPAAHGFVAGRSIVSNARPHVGADVVVNLD
LKDFFPTVSFPRVKGALRHLGYSESVATALALVCTEPEVDEVVLDGTTW
YVARGERFLPQGSPCSPAITNLLCRRLDRRLHGLAQALGFVYTRYADDL
TFSGRGEAAESKRVGKLLRGAADIVAHEGFVVHPDKTRVMRRGRRQEVT
GVVVNDKTSVPRDELRKFRATLYQIEKDGPADKRWGNGGDVLAAVHGYA
CFVAMVDPSRGQPLLARARALLAKHGGPSKPPGGSGPRAPTPVQPTANA
PEAPKPVAPATPAAPAKKGWKLF

In some embodiments, the present disclosure provides reverse transcriptases, and prime editors (e.g. fusion proteins or prime editors in which each component is provided in trans) comprising reverse transcriptases, wherein the reverse transcriptase is a Vc95 reverse transcriptase of SEQ ID NO: 58, or a Vc95 reverse transcriptase variant having at least 70%, at least 75%, at least 80%, at least 85%, at least 90%, at least 95%, at least 96%, at least 97%, at least 98%, or at least 99% sequence identity with SEQ ID NO: 58, wherein the Vc95 reverse transcriptase variant comprises one or more mutations selected from the group consisting of L11M, S75A, V97M, N146D, and N245T relative to SEQ ID NO: 58. In some embodiments, the Vc95 reverse transcriptase variant comprises the mutations L11M, S75A, V97M, N146D, and N245T.

In certain embodiments, the Vc95 reverse transcriptase variant comprises the amino acid sequence of SEQ ID NO: 240, or an amino acid sequence at least 70%, at least 75%, at least 80%, at least 85%, at least 90%, at least 95%, at least 96%, at least 97%, at least 98%, or at least 99% identical to SEQ ID NO: 240, wherein the amino acid sequence comprises at least one of residues 11M, 75A, 97M, 146D, and 245T:

Vc95 RT variant-25.8:
(SEQ ID NO: 240)
NILTTLREQLMTNNVIMPQEFERLEVRGSHAYKVYSIPKRKAGRRTIAH
PSSKLKICQRHLNAILNPLLKVHDASYAYVKGRSIKDNALVHSHSAYML
KMDFQNFFNSITPTILRQCLIQNDILLSVNELEKLEQLIFWNPSKKRDG
KLILSVGSPISPLISNAIMYPFDKIINDICTKHGINYTRYADDITFSTN
IKNTLNKLPEIVEQLIIQTYAGRIIINKRKTVFSSKKHNRHVTGITLTT
DSKISIGRSRKRYISSLVFKYINKNLDIDEINHMKGMLAFAYNIEPIYI
HRLSHKYKVNIVEKILRGSN

In some embodiments, the present disclosure provides reverse transcriptases, and prime editors (e.g. fusion proteins or prime editors in which each component is provided in trans) comprising reverse transcriptases, wherein the reverse transcriptase is a Gs reverse transcriptase of SEQ ID NO: 60, or a Gs reverse transcriptase variant having at least 70%, at least 75%, at least 80%, at least 85%, at least 90%, at least 95%, at least 96%, at least 97%, at least 98%, or at least 99% sequence identity with SEQ ID NO: 60, wherein the Gs reverse transcriptase variant comprises one or more mutations selected from the group consisting of N12D, A16E, A16V, L17P, V20G, L37R, L37P, R38H, Y40C, 141N, 141S, W45R, I67T, I67R, G72E, G73V, G78V, Q93R, A123V, Y126F, E129G, K162N, P190L, D206V, R233K, A234V, R263G, P264S, R267M, K279E, R287I, R291K, P309T, R344S, R358S, R360S, E363G, V374A, and Q412H relative to SEQ ID NO: 60. In some embodiments, the Gs reverse transcriptase variant comprises two or more, three or more, four or more, five or more, six or more, seven or more, eight or more, nine or more, or ten or more of these mutations.

In some embodiments, the Gs reverse transcriptase variant comprises any one of the following groups of mutations relative to the amino acid sequence of SEQ ID NO: 60: L17P and D206V; N12D, L37R, and G78V; A16E, L37P, and A123V; A16V, R38H, W45R, Y126F, and Q412H; A16V, R38H, W45R, and R291K; N12D, L37R, G72E, E129G, P264S, R344S, and R360S; N12D, Y40C, I67T, G73V, Q93R, R287I, and R358S; N12D, Y40C, I67T, G73V, Q93R, and R358S; N12D, 141N, P190L, A234V, and K279E; N12D, L37R, R267M, P309T, R358S, and E363G; A16V, V20G, I41S, R233K, and P264S; L17P, V20G, I41S, I67R, R263G, P264S, and V374A; or L17P, V20G, 141S, 167R, K162N, R263G, and P264S.

In certain embodiments, the Gs reverse transcriptase variant comprises the amino acid sequence of any one of SEQ ID NOs: 159-171, or an amino acid sequence at least 70%, at least 75%, at least 80%, at least 85%, at least 90%, at least 95%, at least 96%, at least 97%, at least 98%, or at least 99% identical to any one of SEQ ID NOs: 159-171, wherein the amino acid sequence comprises at least one of residues 12D, 16E, 16V, 17P, 20G, 37R, 37P, 38H, 40C, 41N, 41S, 45R, 67T, 67R, 72E, 73V, 78V, 93R, 123V, 126F, 129G, 162N, 190L, 206V, 233K, 234V, 263G, 264S, 267M, 279E, 287I, 291K, 309T, 344S, 358S, 360S, 363G, 374A, and 412H:

Gs variants comprising: L17P + D206V
(SEQ ID NO: 159)
EANQGAPGIDGVSTDQLRDYIRAHWSTIHAQLLAGTYRPAPVRRVEIPKPGGGTRQL
GIPTVVDRLIQQAILQELTPIFDPDFSSSSFGFRPGRNAHDAVRQAQGYIQEGYRYVV
DMDLEKFFDRVNHDILMSRVARKVKDKRVLKLIRAYLQAGVMIEGVKVQTEEGTP
QGGPLSPLLANILLDVLDKELEKRGLKFCRYADDCNIYVKSLRAGQRVKQSIQRFLE
KTLKLKVNEEKSAVDRPWKRAFLGFSFTPERKARIRLAPRSIQRLKQRIRQLTNPNWS
ISMPERIHRVNQYVMGWIGYFRLVETPSVLQTIEGWIRRRLRLCQWLQWKRVRTRIR
ELRALGLKETAVMEIANTRKGAWRTTKTPQLHQALGKTYWTAQGLKSLTQRYFELRQG
Gs variant N12D + L37R + G78V
(SEQ ID NO: 160)
ALLERILARDDLITALKRVEANQGAPGIDGVSTDQRRDYIRAHWSTIHAQLLAGTYR
PAPVRRVEIPKPGGGTRQLVIPTVVDRLIQQAILQELTPIFDPDFSSSSFGFRPGRNAHD
AVRQAQGYIQEGYRYVVDMDLEKFFDRVNHDILMSRVARKVKDKRVLKLIRAYLQ
AGVMIEGVKVQTEEGTPQGGPLSPLLANILLDDLDKELEKRGLKFCRYADDCNIYVK
SLRAGQRVKQSIQRFLEKTLKLKVNEEKSAVDRPWKRAFLGFSFTPERKARIRLAPRS
IQRLKQRIRQLTNPNWSISMPERIHRVNQYVMGWIGYFRLVETPSVLQTIEGWIRRRL
RLCQWLQWKRVRTRIRELRALGLKETAVMEIANTRKGAWRTTKTPQLHQALGKTY
WTAQGLKSLTQRYFELRQG
Gs A16E + L37P + A123V
(SEQ ID NO: 161)
ALLERILARDNLITELKRVEANQGAPGIDGVSTDQPRDYIRAHWSTIHAQLLAGTYRP
APVRRVEIPKPGGGTRQLGIPTVVDRLIQQAILQELTPIFDPDFSSSSFGFRPGRNAHDA
VRQVQGYIQEGYRYVVDMDLEKFFDRVNHDILMSRVARKVKDKRVLKLIRAYLQA
GVMIEGVKVQTEEGTPQGGPLSPLLANILLDDLDKELEKRGLKFCRYADDCNIYVKS
LRAGQRVKQSIQRFLEKTLKLKVNEEKSAVDRPWKRAFLGFSFTPERKARIRLAPRSI
QRLKQRIRQLTNPNWSISMPERIHRVNQYVMGWIGYFRLVETPSVLQTIEGWIRRRL
RLCQWLQWKRVRTRIRELRALGLKETAVMEIANTRKGAWRTTKTPQLHQALGKTY
WTAQGLKSLTQRYFELRQG
Gs variant A16V + R38H + W45R + Y126F + Q412H
(SEQ ID NO: 162)
ALLERILARDNLITVLKRVEANQGAPGIDGVSTDQLHDYIRAHRSTIHAQLLAGTYRP
APVRRVEIPKPGGGTRQLGIPTVVDRLIQQAILQELTPIFDPDFSSSSFGFRPGRNAHDA
VRQAQGFIQEGYRYVVDMDLEKFFDRVNHDILMSRVARKVKDKRVLKLIRAYLQA
GVMIEGVKVQTEEGTPQGGPLSPLLANILLDDLDKELEKRGLKFCRYADDCNIYVKS
LRAGQRVKQSIQRFLEKTLKLKVNEEKSAVDRPWKRAFLGFSFTPERKARIRLAPRSI
QRLKQRIRQLTNPNWSISMPERIHRVNQYVMGWIGYFRLVETPSVLQTIEGWIRRRL
RLCQWLQWKRVRTRIRELRALGLKETAVMEIANTRKGAWRTTKTPQLHQALGKTY
WTAQGLKSLTHRYFELRQG
Gs A16V + R38H + W45R + R291K
(SEQ ID NO: 163)
ALLERILARDNLITVLKRVEANQGAPGIDGVSTDQLHDYIRAHRSTIHAQLLAGTYRP
APVRRVEIPKPGGGTRQLGIPTVVDRLIQQAILQELTPIFDPDFSSSSFGFRPGRNAHDA
VRQAQGYIQEGYRYVVDMDLEKFFDRVNHDILMSRVARKVKDKRVLKLIRAYLQA
GVMIEGVKVQTEEGTPQGGPLSPLLANILLDDLDKELEKRGLKFCRYADDCNIYVKS
LRAGQRVKQSIQRFLEKTLKLKVNEEKSAVDRPWKRAFLGFSFTPERKARIRLAPRSI
QKLKQRIRQLTNPNWSISMPERIHRVNQYVMGWIGYFRLVETPSVLQTIEGWIRRRL
RLCQWLQWKRVRTRIRELRALGLKETAVMEIANTRKGAWRTTKTPQLHQALGKTY
WTAQGLKSLTQRYFELRQG
Gs variant 814 N12D + L37R + G72E + E129G + P264S + R344S + R360S
(SEQ ID NO: 164)
ALLERILARDDLITALKRVEANQGAPGIDGVSTDQRRDYIRAHWSTIHAQLLAGTYR
PAPVRRVEIPKPGEGTRQLGIPTVVDRLIQQAILQELTPIFDPDFSSSSFGFRPGRNAHD
AVRQAQGYIQGGYRYVVDMDLEKFFDRVNHDILMSRVARKVKDKRVLKLIRAYLQ
AGVMIEGVKVQTEEGTPQGGPLSPLLANILLDDLDKELEKRGLKFCRYADDCNIYVK
SLRAGQRVKQSIQRFLEKTLKLKVNEEKSAVDRSWKRAFLGFSFTPERKARIRLAPRS
IQRLKQRIRQLTNPNWSISMPERIHRVNQYVMGWIGYFRLVETPSVLQTIEGWIRSRL
RLCQWLQWKRVRTSIRELRALGLKETAVMEIANTRKGAWRTTKTPQLHQALGKTY
WTAQGLKSLTQRYFELRQG
Gs variant 815 N12D + Y40C + 167T + G73V + Q93R + R2871 + R358S
(SEQ ID NO: 165)
ALLERILARDDLITALKRVEANQGAPGIDGVSTDQLRDCIRAHWSTIHAQLLAGTYRP
APVRRVETPKPGGVTRQLGIPTVVDRLIQQAILRELTPIFDPDFSSSSFGFRPGRNAHD
AVRQAQGYIQEGYRYVVDMDLEKFFDRVNHDILMSRVARKVKDKRVLKLIRAYLQ
AGVMIEGVKVQTEEGTPQGGPLSPLLANILLDDLDKELEKRGLKFCRYADDCNIYVK
SLRAGQRVKQSIQRFLEKTLKLKVNEEKSAVDRPWKRAFLGFSFTPERKARIRLAPISI
QRLKQRIRQLTNPNWSISMPERIHRVNQYVMGWIGYFRLVETPSVLQTIEGWIRRRL
RLCQWLQWKRVSTRIRELRALGLKETAVMEIANTRKGAWRTTKTPQLHQALGKTY
WTAQGLKSLTQRYFELRQG
(SEQ ID NO: 166)
Gs variant 816 N12D + Y40C + 167T + G73V + Q93R + R358S
ALLERILARDDLITALKRVEANQGAPGIDGVSTDQLRDCIRAHWSTIHAQLLAGTYRP
APVRRVETPKPGGVTRQLGIPTVVDRLIQQAILRELTPIFDPDFSSSSFGFRPGRNAHD
AVRQAQGYIQEGYRYVVDMDLEKFFDRVNHDILMSRVARKVKDKRVLKLIRAYLQ
AGVMIEGVKVQTEEGTPQGGPLSPLLANILLDDLDKELEKRGLKFCRYADDCNIYVK
SLRAGQRVKQSIQRFLEKTLKLKVNEEKSAVDRPWKRAFLGFSFTPERKARIRLAPRS
IQRLKQRIRQLTNPNWSISMPERIHRVNQYVMGWIGYFRLVETPSVLQTIEGWIRRRL
RLCQWLQWKRVSTRIRELRALGLKETAVMEIANTRKGAWRTTKTPQLHQALGKTY
WTAQGLKSLTQRYFELRQG
Gs variant 817 N12D + I41N + P190L + A234V + K279E
(SEQ ID NO: 167)
ALLERILARDDLITALKRVEANQGAPGIDGVSTDQLRDYNRAHWSTIHAQLLAGTYR
PAPVRRVEIPKPGGGTRQLGIPTVVDRLIQQAILQELTPIFDPDFSSSSFGFRPGRNAHD
AVRQAQGYIQEGYRYVVDMDLEKFFDRVNHDILMSRVARKVKDKRVLKLIRAYLQ
AGVMIEGVKVQTEEGTLQGGPLSPLLANILLDDLDKELEKRGLKFCRYADDCNIYVK
SLRVGQRVKQSIQRFLEKTLKLKVNEEKSAVDRPWKRAFLGFSFTPEREARIRLAPRS
IQRLKQRIRQLTNPNWSISMPERIHRVNQYVMGWIGYFRLVETPSVLQTIEGWIRRRL
RLCQWLQWKRVRTRIRELRALGLKETAVMEIANTRKGAWRTTKTPQLHQALGKTY
WTAQGLKSLTQRYFELRQG
Gs variant 818 N12D + L37R + R267M + P309T + R358S + E363G
(SEQ ID NO: 168)
ALLERILARDDLITALKRVEANQGAPGIDGVSTDQRRDYIRAHWSTIHAQLLAGTYR
PAPVRR VEIPKPGGGTRQLGIPTVVDRLIQQAILQELTPIFDPDFSSSSFGFRPGRNAHD
AVRQAQGYIQEGYRYVVDMDLEKFFDRVNHDILMSRVARKVKDKRVLKLIRAYLQ
AGVMIEGVKVQTEEGTPQGGPLSPLLANILLDDLDKELEKRGLKFCRYADDCNIYVK
SLRAGQRVKQSIQRFLEKTLKLKVNEEKSAVDRPWKMAFLGFSFTPERKARIRLAPR
SIQRLKQRIRQLTNPNWSISMTERIHRVNQYVMGWIGYFRLVETPSVLQTIEGWIRRR
LRLCQWLQWKRVSTRIRGLRALGLKETAVMEIANTRKGAWRTTKTPQLHQALGKT
YWTAQGLKSLTQRYFELRQG
Gs variant 819 A16V + V20G + 141S + R233K + P264S
(SEQ ID NO: 169)
ALLERILARDNLITVLKRGEANQGAPGIDGVSTDQLRDYSRAHWSTIHAQLLAGTYR
PAPVRRVEIPKPGGGTRQLGIPTVVDRLIQQAILQELTPIFDPDFSSSSFGFRPGRNAHD
AVRQAQGYIQEGYRYVVDMDLEKFFDRVNHDILMSRVARKVKDKRVLKLIRAYLQ
AGVMIEGVKVQTEEGTPQGGPLSPLLANILLDDLDKELEKRGLKFCRYADDCNIYVK
SLKAGQRVKQSIQRFLEKTLKLKVNEEKSAVDRSWKRAFLGFSFTPERKARIRLAPR
SIQRLKQRIRQLTNPNWSISMPERIHRVNQYVMGWIGYFRLVETPSVLQTIEGWIRRR
LRLCQWLQWKRVRTRIRELRALGLKETAVMEIANTRKGAWRTTKTPQLHQALGKT
YWTAQGLKSLTQRYFELRQG
Gs variant 820 L17P + V20G + 141S + 167R + R263G + P264S + V374A
(SEQ ID NO: 170)
ALLERILARDNLITAPKRGEANQGAPGIDGVSTDQLRDYSRAHWSTIHAQLLAGTYR
PAPVRRVERPKPGGGTRQLGIPTVVDRLIQQAILQELTPIFDPDFSSSSFGFRPGRNAH
DAVRQAQGYIQEGYRYVVDMDLEKFFDRVNHDILMSRVARKVKDKRVLKLIRAYL
QAGVMIEGVKVQTEEGTPQGGPLSPLLANILLDDLDKELEKRGLKFCRYADDCNIYV
KSLRAGQRVKQSIQRFLEKTLKLKVNEEKSAVDGSWKRAFLGFSFTPERKARIRLAP
RSIQRLKQRIRQLTNPNWSISMPERIHRVNQYVMGWIGYFRLVETPSVLQTIEGWIRR
RLRLCQWLQWKRVRTRIRELRALGLKETAAMEIANTRKGAWRTTKTPQLHQALGK
TYWTAQGLKSLTQRYFELRQG
Gs variant 821 L17P + V20G + 141S + 167R + K162N + R263G + P264S
(SEQ ID NO: 171)
ALLERILARDNLITAPKRGEANQGAPGIDGVSTDQLRDYSRAHWSTIHAQLLAGTYR
PAPVRRVERPKPGGGTRQLGIPTVVDRLIQQAILQELTPIFDPDFSSSSFGFRPGRNAH
DAVRQAQGYIQEGYRYVVDMDLEKFFDRVNHDILMSRVARKVKDKNVLKLIRAYL
QAGVMIEGVKVQTEEGTPQGGPLSPLLANILLDDLDKELEKRGLKFCRYADDCNIYV
KSLRAGQRVKQSIQRFLEKTLKLKVNEEKSAVDGSWKRAFLGFSFTPERKARIRLAP
RSIQRLKQRIRQLTNPNWSISMPERIHRVNQYVMGWIGYFRLVETPSVLQTIEGWIRR
RLRLCQWLQWKRVRTRIRELRALGLKETAVMEIANTRKGAWRTTKTPQLHQALGK
TYWTAQGLKSLTQRYFELRQG

### Nuclear Localization Sequences (NLS)

In various embodiments, the prime editors used in the complexes and methods described herein may comprise one or more nuclear localization sequences (NLS), which help promote translocation of a protein into the cell nucleus. Such sequences are well-known in the art and can include the following examples:

| | | SEQ |
|---|---|---|
| DESCRIPTION | SEQUENCE | ID NO: |
| NLS OF SV40 LARGE | PKKKRKV | 94 |
| T-AG | | |
| NLS | MKRTADGSEFESPKKKRKV | 95 |
| NLS | MDSLLMNRRKFLYQFKNVRWA | 99 |
| | KGRRETYLC | |
| NLS OF | AVKRPAATKKAGQAKKKKLD | 100 |
| NUCLEOPLASMIN | | |
| NLS OF EGL-13 | MSRRRKANPTKLSENAKKLAK | 101 |
| | EVEN | |
| NLS OF C-MYC | PAAKRVKLD | 98 |
| NLS OF TUS-PROTEIN | KLKIKRPVK | 102 |
| NLS OF POLYOMA | VSRKRPRP | 103 |
| LARGE T-AG | | |
| NLS OF HEPATITIS D | EGAPPAKRAR | 104 |
| VIRUS ANTIGEN | | |
| NLS OF MURINE P53 | PPQPKKKPLDGE | 105 |
| NLS OF PE1 AND PE2 | SGGSKRTADGSEFEPKKKRKV | 96 |
| BIPARTITE SV40 NLS | KRTADGSEFESPKKKRKV | 97 |

The NLS examples above are non-limiting. The prime editors used in the presently described complexes and methods may comprise any known NLS sequence, including any of those described in Cokol et al., “Finding nuclear localization signals,” EMBO Rep., 2000, 1(5): 411-415 and Freitas et al., “Mechanisms and Signals for the Nuclear Import of Proteins,” Current Genomics, 2009, 10(8): 550-7, each of which are incorporated herein by reference.

In various embodiments, the fusion proteins used in the complexes and methods described herein further comprise one or more (and preferably at least two) nuclear localization sequences. In certain embodiments, the fusion proteins comprise at least two NLSs. In embodiments with at least two NLSs, the NLSs can be the same NLSs or they can be different NLSs. In some embodiments, one or more of the NLSs are bipartite NLSs (“bpNLS”). In certain embodiments, the disclosed fusion proteins comprise two bipartite NLSs. In some embodiments, the disclosed fusion proteins comprise more than two bipartite NLSs.

The location of the NLS fusion can be at the N-terminus, the C-terminus, or within a sequence of a fusion protein (e.g., inserted between the encoded napDNAbp component (e.g., Cas9) and a polymerase domain (e.g., a reverse transcriptase).

The NLSs may be any known NLS sequence in the art. The NLSs may also be any future-discovered NLSs for nuclear localization. The NLSs also may be any naturally-occurring NLS, or any non-naturally occurring NLS (e.g., an NLS with one or more desired mutations).

The term “nuclear localization sequence” or “NLS” refers to an amino acid sequence that promotes import of a protein into the cell nucleus, for example, by nuclear transport. Nuclear localization sequences are known in the art and would be apparent to the skilled artisan. For example, NLS sequences are described in Plank et al., International PCT application PCT/EP2000/011690, filed Nov. 23, 2000, published as WO/2001/038547 on May 31, 2001, the contents of which are incorporated herein by reference. In some embodiments, an NLS comprises the amino acid sequence PKKKRKV (SEQ ID NO: 94), MDSLLMNRRKFLYQFKNVRWAKGRRETYLC (SEQ ID NO: 99), KRTADGSEFESPKKKRKV (SEQ ID NO: 97), or KRTADGSEFEPKKKRKV (SEQ ID NO: 106). In other embodiments, an NLS comprises the amino acid sequences NLSKRPAAIKKAGQAKKKK (SEQ ID NO: 107), PAAKRVKLD (SEQ ID NO: 98), RQRRNELKRSF (SEQ ID NO: 108), or NQSSNFGPMKGGNFGGRSSGPYGGGGQYFAKPRNQGGY (SEQ ID NO: 109).

In one aspect of the disclosure, a prime editor or other fusion protein may be modified with one or more nuclear localization sequences (NLS), preferably at least two NLSs. In certain embodiments, the fusion proteins are modified with two or more NLSs. The disclosure contemplates the use of any nuclear localization sequence known in the art at the time of the disclosure, or any nuclear localization sequence that is identified or otherwise made available in the state of the art after the time of the instant filing. A representative nuclear localization sequence is a peptide sequence that directs the protein to the nucleus of the cell in which the sequence is expressed. A nuclear localization signal is predominantly basic, can be positioned almost anywhere in a protein's amino acid sequence, generally comprises a short sequence of four amino acids (Autieri & Agrawal, (1998) J. Biol. Chem. 273:14731-37, incorporated herein by reference) to eight amino acids, and is typically rich in lysine and arginine residues (Magin et al., (2000) Virology 274:11-16, incorporated herein by reference). Nuclear localization sequences often comprise proline residues. A variety of nuclear localization sequences have been identified and have been used to effect transport of biological molecules from the cytoplasm to the nucleus of a cell. See, e.g., Tinland et al., (1992) Proc. Natl. Acad. Sci. U.S.A. 89:7442-46; Moede et al., (1999) FEBS Lett. 461:229-34, which is incorporated herein by reference. Translocation is currently thought to involve nuclear pore proteins.

Most NLSs can be classified in three general groups: (i) a monopartite NLS exemplified by the SV40 large T antigen NLS (PKKKRKV (SEQ ID NO: 94)); (ii) a bipartite motif consisting of two basic domains separated by a variable number of spacer amino acids and exemplified by the Xenopus nucleoplasmin NLS (KRXXXXXXXXXXKKKL (SEQ ID NO: 110)); and (iii) noncanonical sequences such as M9 of the hnRNP Al protein, the influenza virus nucleoprotein NLS, and the yeast Gal4 protein NLS (Dingwall and Laskey 1991).

Nuclear localization sequences appear at various points in the amino acid sequences of proteins. NLS have been identified at the N-terminus, the C-terminus, and in the central region of proteins. Thus, the disclosure provides fusion proteins that may be modified with one or more NLSs at the C-terminus and/or the N-terminus, as well as at internal regions of the fusion protein. The residues of a longer sequence that do not function as component NLS residues should be selected so as not to interfere, for example, tonically or sterically, with the nuclear localization signal itself. Therefore, although there are no strict limits on the composition of an NLS-comprising sequence, in practice, such a sequence can be functionally limited in length and composition.

The present disclosure contemplates any suitable means by which to modify a fusion protein to include one or more NLSs. In one aspect, the fusion proteins may be engineered to express a fusion protein that is translationally fused at its N-terminus or its C-terminus (or both) to one or more NLSs, i.e., to form a prime editor-NLS fusion construct. In other embodiments, a fusion protein-encoding nucleotide sequence may be genetically modified to incorporate a reading frame that encodes one or more NLSs in an internal region of the encoded prime editor. In addition, the NLSs may include various amino acid linkers or spacer regions encoded between the prime editor and the N-terminally, C-terminally, or internally-attached NLS amino acid sequence, e.g., and in the central region of proteins. Thus, the present disclosure also provides for nucleotide constructs, vectors, and host cells for expressing fusion proteins that comprise a prime editor and one or more NLSs, among other components.

The prime editors described herein may also comprise nuclear localization sequences that are linked to a prime editor through one or more linkers, e.g., a polymeric, amino acid, nucleic acid, polysaccharide, chemical, or nucleic acid linker element. The linkers within the contemplated scope of the disclosure are not intended to have any limitations and can be any suitable type of molecule (e.g., polymer, amino acid, polysaccharide, nucleic acid, lipid, or any synthetic chemical linker domain) and can be joined to the prime editor by any suitable strategy that effectuates forming a bond (e.g., covalent linkage, hydrogen bonding) between the prime editor and the one or more NLSs.

### Linkers

The prime editors used in the complexes and methods described herein may include one or more linkers. As defined above, the term “linker,” as used herein, refers to a chemical group or a molecule linking two molecules or moieties, e.g., a binding domain and a cleavage domain of a nuclease. In some embodiments, a linker joins a gRNA binding domain of an RNA-programmable nuclease and a polymerase (e.g., a reverse transcriptase). In some embodiments, a linker joins a Cas9 nickase and a reverse transcriptase. Typically, the linker is positioned between, or flanked by, two groups, molecules, or other moieties and connected to each one via a covalent bond, thus connecting the two. In some embodiments, the linker is an amino acid or a plurality of amino acids (e.g., a peptide or protein). In some embodiments, the linker is an organic molecule, group, polymer, or chemical moiety. In some embodiments, the linker is 5-100 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated.

The linker may be as simple as a covalent bond, or it may be a polymeric linker many atoms in length. In certain embodiments, the linker is a polypeptide, or amino acid-based. In other embodiments, the linker is not peptide-like. In certain embodiments, the linker is a covalent bond (e.g., a carbon-carbon bond, disulfide bond, carbon-heteroatom bond, etc.). In certain embodiments, the linker is a carbon-nitrogen bond of an amide linkage. In certain embodiments, the linker is a cyclic or acyclic, substituted or unsubstituted, branched or unbranched, aliphatic or heteroaliphatic linker. In certain embodiments, the linker is polymeric (e.g., polyethylene, polyethylene glycol, polyamide, polyester, etc.). In certain embodiments, the linker comprises a monomer, dimer, or polymer of aminoalkanoic acid. In certain embodiments, the linker comprises an aminoalkanoic acid (e.g., glycine, ethanoic acid, alanine, beta-alanine, 3-aminopropanoic acid, 4-aminobutanoic acid, 5-pentanoic acid, etc.). In certain embodiments, the linker comprises a monomer, dimer, or polymer of aminohexanoic acid (Ahx). In certain embodiments, the linker is based on a carbocyclic moiety (e.g., cyclopentane, cyclohexane). In other embodiments, the linker comprises a polyethylene glycol moiety (PEG). In other embodiments, the linker comprises amino acids. In certain embodiments, the linker comprises a peptide. In certain embodiments, the linker comprises an aryl or heteroaryl moiety. In certain embodiments, the linker is based on a phenyl ring. The linker may include functionalized moieties to facilitate attachment of a nucleophile (e.g., thiol, amino) from the peptide to the linker. Any electrophile may be used as part of the linker. Exemplary electrophiles include, but are not limited to, activated esters, activated amides, Michael acceptors, alkyl halides, aryl halides, acyl halides, and isothiocyanates.

In some other embodiments, the linker comprises the amino acid sequence (GGGGS)ₙ (SEQ ID NO: 84), (G)ₙ (SEQ ID NO: 85), (EAAAK)ₙ (SEQ ID NO: 86), (GGS)ₙ (SEQ ID NO: 87), (SGGS)ₙ (SEQ ID NO: 81), (XP)ₙ (SEQ ID NO: 88), or any combination thereof, wherein n is independently an integer between 1 and 30, and wherein X is any amino acid. In some embodiments, the linker comprises the amino acid sequence (GGS)ₙ (SEQ ID NO: 87), wherein n is 1, 3, or 7. In some embodiments, the linker comprises the amino acid sequence SGSETPGTSESATPES (SEQ ID NO: 89). In some embodiments, the linker comprises the amino acid sequence SGGSSGGSSGSETPGTSESATPESSGGSSGGS (SEQ ID NO: 90). In some embodiments, the linker comprises the amino acid sequence SGGSGGSGGS (SEQ ID NO: 91). In some embodiments, the linker comprises the amino acid sequence SGGS (SEQ ID NO: 82). In other embodiments, the linker comprises the amino acid sequence SGGSSGGSSGSETPGTSESATPESAGSYPYDVPDYAGSAAPAAKKKKLDGSGSGGSS GGS (SEQ ID NO: 83, 60AA). In some embodiments, the linker comprises the amino acid sequence GGS, GGSGGS (SEQ ID NO: 92), GGSGGSGGS (SEQ ID NO: 93), SGGSSGGSSGSETPGTSESATPESSGGSSGGSS (SEQ ID NO: 80), SGSETPGTSESATPES (SEQ ID NO: 89), or SGGSSGGSSGSETPGTSESATPESAGSYPYDVPDYAGSAAPAAKKKKLDGSGSGGSS GGS (SEQ ID NO: 83).

In certain embodiments, linkers may be used to link any of the peptides or peptide domains or moieties of the invention (e.g., a napDNAbp linked or fused to a reverse transcriptase domain, and/or a napDNAbp linked to one or more NLS). Any of the domains of the fusion proteins used in the complexes and methods described herein may also be connected to one another through any of the presently described linkers.

### Additional Prime Editor Domains

### A. Flap Endonucleases (e.g., FEN1)

In various embodiments, the prime editors described herein may comprise one or more flap endonucleases (e.g., FEN1), which refers to an enzyme that catalyzes the removal of 5′ single stranded DNA flaps (provided in trans or fused to the PE fusion proteins). These are naturally occurring enzymes that process the removal of 5′ flaps formed during cellular processes, including DNA replication. The prime editors described herein may utilize endogenously supplied flap endonucleases or those provided in trans to remove the 5′ flap of endogenous DNA formed at the target site during prime editing. Flap endonucleases are known in the art and are described, for example, in Patel et al., “Flap endonucleases pass 5′-flaps through a flexible arch using a disorder-thread-order mechanism to confer specificity for free 5′-ends,” Nucleic Acids Research, 2012, 40(10): 4507-4519 and Tsutakawa et al., “Human flap endonuclease structures, DNA double-base flipping, and a unified understanding of the FEN1 superfamily,” Cell, 2011, 145(2): 198-211 (each of which are incorporated herein by reference). An exemplary flap endonuclease is FEN1, which can be represented by the following amino acid sequence:

| Description | Sequence | SEQ ID NO: |
|---|---|---|
| FEN1 | MGIQGLAKLIADVAPSAIRENDIK | SEQ ID NO: |
| Wild type | SYFGRKVAIDASMSIYQFLIAVRQ | 112 |
| (wt) | GGDVLQNEEGETTSHLMGMFYRTI | |
| | RMMENGIKPVYVFDGKPPQLKSGE | |
| | LAKRSERRAEAEKQLQQAQAAGAE | |
| | QEVEKFTKRLVKVTKQHNDECKHL | |
| | LSLMGIPYLDAPSEAEASCAALVK | |
| | AGKVYAAATEDMDCLTFGSPVLMR | |
| | HLTASEAKKLPIQEFHLSRILQEL | |
| | GLNQEQFVDLCILLGSDYCESIRG | |
| | IGPKRAVDLIQKHKSIEEIVRRLD | |
| | PNKYPVPENWLHKEAHQLFLEPEV | |
| | LDPESVELKWSEPNEEELIKFMCG | |
| | EKQFSEERIRSGVKRLSKSRQGST | |
| | QGRLDDFFKVTGSLSSAKRKEPEP | |
| | KGSTKKKAKTGAAGKFKRGK | |

The flap endonucleases may also include any FEN1 variant, mutant, or other flap endonuclease ortholog, homolog, or variant. Non-limiting FEN1 variant examples are as follows:

| Description | Sequence | SEQ ID NO: |
|---|---|---|
| FEN1 | MGIQGLAKLIADVAPSAIRENDIKSYFGRKVAIDASMSI | SEQ ID NO: 113 |
| K168R | YQFLIAVRQGGDVLQNEEGETTSHLMGMFYRTIRMME | |
| (relative to | NGIKPVYVFDGKPPQLKSGELAKRSERRAEAEKQLQQ | |
| FEN1 wt) | AQAAGAEQEVEKFTKRLVKVTKQHNDECKHLLSLMGI | |
| | PYLDAPSEAEASCAALVRAGKVYAAATEDMDCLTFGS | |
| | PVLMRHLTASEAKKLPIQEFHLSRILQELGLNQEQFVD | |
| | LCILLGSDYCESIRGIGPKRAVDLIQKHKSIEEIVRRLDP | |
| | NKYPVPENWLHKEAHQLFLEPEVLDPESVELKWSEPN | |
| | EEELIKFMCGEKQFSEERIRSGVKRLSKSRQGSTQGRLD | |
| | DFFKVTGSLSSAKRKEPEPKGSTKKKAKTGAAGKFKR | |
| | GK | |
| FEN1 | MGIQGLAKLIADVAPSAIRENDIKSYFGRKVAIDASMSI | SEQ ID NO: 114 |
| S187A | YQFLIAVRQGGDVLQNEEGETTSHLMGMFYRTIRMME | |
| (relative to | NGIKPVYVFDGKPPQLKSGELAKRSERRAEAEKQLQQ | |
| FEN1 wt) | AQAAGAEQEVEKFTKRLVKVTKQHNDECKHLLSLMGI | |
| | PYLDAPSEAEASCAALVKAGKVYAAATEDMDCLTFG | |
| | APVLMRHLTASEAKKLPIQEFHLSRILQELGLNQEQFV | |
| | DLCILLGSDYCESIRGIGPKRAVDLIQKHKSIEEIVRRLD | |
| | PNKYPVPENWLHKEAHQLFLEPEVLDPESVELKWSEP | |
| | NEEELIKFMCGEKQFSEERIRSGVKRLSKSRQGSTQGRL | |
| | DDFFKVTGSLSSAKRKEPEPKGSTKKKAKTGAAGKFK | |
| | RGK | |
| FEN1 | MGIQGLAKLIADVAPSAIRENDIKSYFGRKVAIDASMSI | SEQ ID NO: 115 |
| K354R | YQFLIAVRQGGDVLQNEEGETTSHLMGMFYRTIRMME | |
| (relative to | NGIKPVYVFDGKPPQLKSGELAKRSERRAEAEKQLQQ | |
| FEN1 wt) | AQAAGAEQEVEKFTKRLVKVTKQHNDECKHLLSLMGI | |
| | PYLDAPSEAEASCAALVKAGKVYAAATEDMDCLTFGS | |
| | PVLMRHLTASEAKKLPIQEFHLSRILQELGLNQEQFVD | |
| | LCILLGSDYCESIRGIGPKRAVDLIQKHKSIEEIVRRLDP | |
| | NKYPVPENWLHKEAHQLFLEPEVLDPESVELKWSEPN | |
| | EEELIKFMCGEKQFSEERIRSGVKRLSKSRQGSTQGRLD | |
| | DFFKVTGSLSSARRKEPEPKGSTKKKAKTGAAGKFKR | |
| | GK | |
| GEN1 | MGVNDLWQILEPVKQHIPLRNLGGKTIAVDLSLWVCE | SEQ ID NO: 116 |
| | AQTVKKMMGSVMKPHLRNLFFRISYLTQMDVKLVFV | |
| | MEGEPPKLKADVISKRNQSRYGSSGKSWSQKTGRSHF | |
| | KSVLRECLHMLECLGIPWVQAAGEAEAMCAYLNAGG | |
| | HVDGCLTNDGDTFLYGAQTVYRNFTMNTKDPHVDCY | |
| | TMSSIKSKLGLDRDALVGLAILLGCDYLPKGVPGVGKE | |
| | QALKLIQILKGQSLLQRFNRWNETSCNSSPQLLVTKKL | |
| | AHCSVCSHPGSPKDHERNGCRLCKSDKYCEPHDYEYC | |
| | CPCEWHRTEHDRQLSEVENNIKKKACCCEGFPFHEVIQ | |
| | EFLLNKDKLVKVIRYQRPDLLLFQRFTLEKMEWPNHY | |
| | ACEKLLVLLTHYDMIERKLGSRNSNQLQPIRIVKTRIRN | |
| | GVHCFEIEWEKPEHYAMEDKQHGEFALLTIEEESLFEA | |
| | AYPEIVAVYQKQKLEIKGKKQKRIKPKENNLPEPDEVM | |
| | SFQSHMTLKPTCEIFHKQNSKLNSGISPDPTLPQESISAS | |
| | LNSLLLPKNTPCLNAQEQFMSSLRPLAIQQIKAVSKSLI | |
| | SESSQPNTSSHNISVIADLHLSTIDWEGTSFSNSPAIQRN | |
| | TFSHDLKSEVESELSAIPDGFENIPEQLSCESERYTANIK | |
| | KVLDEDSDGISPEEHLLSGITDLCLQDLPLKERIFTKLSY | |
| | PQDNLQPDVNLKTLSILSVKESCIANSGSDCTSHLSKDL | |
| | PGIPLQNESRDSKILKGDQLLQEDYKVNTSVPYSVSNT | |
| | VVKTCNVRPPNTALDHSRKVDMQTTRKILMKKSVCLD | |
| | RHSSDEQSAPVFGKAKYTTQRMKHSSQKHNSSHFKES | |
| | GHNKLSSPKIHIKETEQCVRSYETAENEESCFPDSTKSS | |
| | LSSLQCHKKENNSGTCLDSPLPLRQRLKLRFQST | |
| ERCC5 | MGVQGLWKLLECSGRQVSPEALEGKILAVDISIWLNQ | SEQ ID NO: 117 |
| | ALKGVRDRHGNSIENPHLLTLFHRLCKLLFFRIRPIFVF | |
| | DGDAPLLKKQTLVKRRQRKDLASSDSRKTTEKLLKTF | |
| | LKRQAIKTAFRSKRDEALPSLTQVRRENDLYVLPPLQE | |
| | EEKHSSEEEDEKEWQERMNQKQALQEEFFHNPQAIDIE | |
| | SEDFSSLPPEVKHEILTDMKEFTKRRRTLFEAMPEESDD | |
| | FSQYQLKGLLKKNYLNQHIEHVQKEMNQQHSGHIRRQ | |
| | YEDEGGFLKEVESRRVVSEDTSHYILIKGIQAKTVAEV | |
| | DSESLPSSSKMHGMSFDVKSSPCEKLKTEKEPDATPPSP | |
| | RTLLAMQAALLGSSSEEELESENRRQARGRNAPAAVD | |
| | EGSISPRTLSAIKRALDDDEDVKVCAGDDVQTGGPGAE | |
| | EMRINSSTENSDEGLKVRDGKGIPFTATLASSSVNSAEE | |
| | HVASTNEGREPTDSVPKEQMSLVHVGTEAFPISDESMI | |
| | KDRKDRLPLESAVVRHSDAPGLPNGRELTPASPTCTNS | |
| | VSKNETHAEVLEQQNELCPYESKFDSSLLSSDDETKCK | |
| | PNSASEVIGPVSLQETSSIVSVPSEAVDNVENVVSFNAK | |
| | EHENFLETIQEQQTTESAGQDLISIPKAVEPMEIDSEESE | |
| | SDGSFIEVQSVISDEELQAEFPETSKPPSEQGEEELVGTR | |
| | EGEAPAESESLLRDNSERDDVDGEPQEAEKDAEDSLHE | |
| | WQDINLEELETLESNLLAQQNSLKAQKQQQERIAATVT | |
| | GQMFLESQELLRLFGIPYIQAPMEAEAQCAILDLTDQTS | |
| | GTITDDSDIWLFGARHVYRNFFNKNKFVEYYQYVDFH | |
| | NQLGLDRNKLINLAYLLGSDYTEGIPTVGCVTAMEILN | |
| | EFPGHGLEPLLKFSEWWHEAQKNPKIRPNPHDTKVKK | |
| | KLRTLQLTPGFPNPAVAEAYLKPVVDDSKGSFLWGKP | |
| | DLDKIREFCQRYFGWNRTKTDESLFPVLKQLDAQQTQ | |
| | LRIDSFFRLAQQEKEDAKRIKSQRLNRAVTCMLRKEKE | |
| | AAASEIEAVSVAMEKEFELLDKAKRKTQKRGITNTLEE | |
| | SSSLKRKRLSDSKRKNTCGGFLGETCLSESSDGSSSEDA | |
| | ESSSLMNVQRRTAAKEPKTSASDSQNSVKEAPVKNGG | |
| | ATTSSSSDSDDDGGKEKMVLVTARSVFGKKRRKLRRA | |
| | RGRKRKT | |

In various embodiments, the prime editors utilized in the complexes and methods contemplated herein may include any flap endonuclease variant of the above-disclosed sequences having an amino acid sequence that is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to any of the above sequences. Other endonucleases that may be utilized by the instant compositions and methods to facilitate removal of the 5′ end single strand DNA flap include, but are not limited to (1) trex 2, (2) exo1 endonuclease (e.g., Keijzers et al., Biosci Rep. 2015, 35(3): e00206) Trex 2

Three prime (3′) repair exonuclease 2 (TREX2)-
human
Accession No. NM_080701
(SEQ ID NO: 118)
MSEAPRAETFVFLDLEATGLPSVEPEIAELSLFAVHRSSLENPEHDESG
ALVLPRVLDKLTLCMCPERPFTAKASEITGLSSEGLARCRKAGFDGAVV
RTLQAFLSRQAGPICLVAHNGFDYDFPLLCAELRRLGARLPRDTVCLDT
LPALRGLDRAHSHGTRARGRQGYSLGSLFHRYFRAEPSAAHSAEGDVHT
LLLIFLHRAAELLAWADEQARGWAHIEPMYLPPDDPSLEA.
Three prime (3′) repair exonuclease 2 (TREX2)-
mouse
Accession No. NM_011907
(SEQ ID NO: 119)
MSEPPRAETFVFLDLEATGLPNMDPEIAEISLFAVHRSSLENPERDDSG
SLVLPRVLDKLTLCMCPERPFTAKASEITGLSSESLMHCGKAGFNGAVV
RTLQGFLSRQEGPICLVAHNGFDYDFPLLCTELQRLGAHLPQDTVCLDT
LPALRGLDRAHSHGTRAQGRKSYSLASLFHRYFQAEPSAAHSAEGDVHT
LLLIFLHRAPELLAWADEQARSWAHIEPMYVPPDGPSLEA.
Three prime (3′) repair exonuclease 2 (TREX2)-
rat
Accession No. NM_001107580
(SEQ ID NO: 120)
MSEPLRAETFVFLDLEATGLPNMDPEIAEISLFAVHRSSLENPERDDSG
SLVLPRVLDKLTLCMCPERPFTAKASEITGLSSEGLMNCRKAAFNDAVV
RTLQGFLSRQEGPICLVAHNGFDYDFPLLCTELQRLGAHLPRDTVCLDT
LPALRGLDRVHSHGTRAQGRKSYSLASLFHRYFQAEPSAAHSAEGDVNT
LLLIFLHRAPELLAWADEQARSWAHIEPMYVPPDGPSLEA.

### ExoI

Human exonuclease 1 (EXO1) has been implicated in many different DNA metabolic processes, including DNA mismatch repair (MMR), micro-mediated end-joining, homologous recombination (HR), and replication. Human EXO1 belongs to a family of eukaryotic nucleases, Rad2/XPG, which also include FEN1 and GEN1. The Rad2/XPG family is conserved in the nuclease domain through species from phage to human. The EXO1 gene product exhibits both 5′ exonuclease and 5′ flap activity. Additionally, EXO1 contains an intrinsic 5′ RNase H activity. Human EXO1 has a high affinity for processing double stranded DNA (dsDNA), nicks, gaps, and pseudo Y structures and can resolve Holliday junctions using its inherit flap activity. Human EXO1 is implicated in MMR and contains conserved binding domains interacting directly with MLH1 and MSH2. EXO1 nucleolytic activity is positively stimulated by PCNA, MutSα (MSH2/MSH6 complex), 14-3-3, MRN, and 9-1-1 complex.

Exonuclease 1 (EXO1) Accession No. NM_003686 (Homo sapiens exonuclease 1
(EXO1), transcript variant 3)-isoform A
(SEQ ID NO: 121)
MGIQGLLQFIKEASEPIHVRKYKGQVVAVDTYCWLHKGAIACAEKLAKGEPTDRYV
GFCMKFVNMLLSHGIKPILVFDGCTLPSKKEVERSRRERRQANLLKGKQLLREGKVS
EARECFTRSINITHAMAHKVIKAARSQGVDCLVAPYEADAQLAYLNKAGIVQAIITE
DSDLLAFGCKKVILKMDQFGNGLEIDQARLGMCRQLGDVFTEEKFRYMCILSGCDY
LSSLRGIGLAKACKVLRLANNPDIVKVIKKIGHYLKMNITVPEDYINGFIRANNTFLY
QLVFDPIKRKLIPLNAYEDDVDPETLSYAGQYVDDSIALQIALGNKDINTFEQIDDYN
PDTAMPAHSRSHSWDDKTCQKSANVSSIWHRNYSPRPESGTVSDAPQLKENPSTVG
VERVISTKGLNLPRKSSIVKRPRSAELSEDDLLSQYSLSFTKKTKKNSSEGNKSLSFSE
VFVPDLVNGPTNKKSVSTPPRTRNKFATFLQRKNEESGAVVVPGTRSRFFCSSDSTDC
VSNKVSIQPLDETAVTDKENNLHESEYGDQEGKRLVDTDVARNSSDDIPNNHIPGDH
IPDKATVFTDEESYSFESSKFTRTISPPTLGTLRSCFSWSGGLGDFSRTPSPSPSTALQQ
FRRKSDSPTSLPENNMSDVSQLKSEESSDDESHPLREEACSSQSQESGEFSLQSSNASK
LSQCSSKDSDSEESDCNIKLLDSQSDQTSKLRLSHFSKKDTPLRNKVPGLYKSSSADS
LSTTKIKPLGPARASGLSKKPASIQKRKHHNAENKPGLQIKLNELWKNFGFKKF.
Exonuclease 1 (EXO1) Accession No. NM_006027 (Homo sapiens exonuclease 1
(EXO1), transcript variant 3)-isoform B
(SEQ ID NO: 122)
MGIQGLLQFIKEASEPIHVRKYKGQVVAVDTYCWLHKGAIACAEKLAKGEPTDRYV
GFCMKFVNMLLSHGIKPILVFDGCTLPSKKEVERSRRERRQANLLKGKQLLREGKVS
EARECFTRSINITHAMAHKVIKAARSQGVDCLVAPYEADAQLAYLNKAGIVQAIITE
DSDLLAFGCKKVILKMDQFGNGLEIDQARLGMCRQLGDVFTEEKFRYMCILSGCDY
LSSLRGIGLAKACKVLRLANNPDIVKVIKKIGHYLKMNITVPEDYINGFIRANNTFLY
QLVFDPIKRKLIPLNAYEDDVDPETLSYAGQYVDDSIALQIALGNKDINTFEQIDDYN
PDTAMPAHSRSHSWDDKTCQKSANVSSIWHRNYSPRPESGTVSDAPQLKENPSTVG
VERVISTKGLNLPRKSSIVKRPRSAELSEDDLLSQYSLSFTKKTKKNSSEGNKSLSFSE
VFVPDLVNGPTNKKSVSTPPRTRNKFATFLQRKNEESGAVVVPGTRSRFFCSSDSTDC
VSNKVSIQPLDETAVTDKENNLHESEYGDQEGKRLVDTDVARNSSDDIPNNHIPGDH
IPDKATVFTDEESYSFESSKFTRTISPPTLGTLRSCFSWSGGLGDFSRTPSPSPSTALQQ
FRRKSDSPTSLPENNMSDVSQLKSEESSDDESHPLREEACSSQSQESGEFSLQSSNASK
LSQCSSKDSDSEESDCNIKLLDSQSDQTSKLRLSHFSKKDTPLRNKVPGLYKSSSADS
LSTTKIKPLGPARASGLSKKPASIQKRKHHNAENKPGLQIKLNELWKNFGFKKDSEK
LPPCKKPLSPVRDNIQLTPEAEEDIFNKPECGRVQRAIFQ.
Exonuclease 1 (EXO1) Accession No. NM_001319224 (Homo sapiens exonuclease 1
(EXO1), transcript variant 4)-isoform C
(SEQ ID NO: 123)
MGIQGLLQFIKEASEPIHVRKYKGQVVAVDTYCWLHKGAIACAEKLAKGEPTDRYV
GFCMKFVNMLLSHGIKPILVFDGCTLPSKKEVERSRRERRQANLLKGKQLLREGKVS
EARECFTRSINITHAMAHKVIKAARSQGVDCLVAPYEADAQLAYLNKAGIVQAIITE
DSDLLAFGCKKVILKMDQFGNGLEIDQARLGMCRQLGDVFTEEKFRYMCILSGCDY
LSSLRGIGLAKACKVLRLANNPDIVKVIKKIGHYLKMNITVPEDYINGFIRANNTFLY
QLVFDPIKRKLIPLNAYEDDVDPETLSYAGQYVDDSIALQIALGNKDINTFEQIDDYN
PDTAMPAHSRSHSWDDKTCQKSANVSSIWHRNYSPRPESGTVSDAPQLKENPSTVG
VERVISTKGLNLPRKSSIVKRPRSELSEDDLLSQYSLSFTKKTKKNSSEGNKSLSFSEV
FVPDLVNGPTNKKSVSTPPRTRNKFATFLQRKNEESGAVVVPGTRSRFFCSSDSTDCV
SNKVSIQPLDETAVTDKENNLHESEYGDQEGKRLVDTDVARNSSDDIPNNHIPGDHIP
DKATVFTDEESYSFESSKFTRTISPPTLGTLRSCFSWSGGLGDFSRTPSPSPSTALQQFR
RKSDSPTSLPENNMSDVSQLKSEESSDDESHPLREEACSSQSQESGEFSLQSSNASKLS
QCSSKDSDSEESDCNIKLLDSQSDQTSKLRLSHFSKKDTPLRNKVPGLYKSSSADSLS
TTKIKPLGPARASGLSKKPASIQKRKHHNAENKPGLQIKLNELWKNFGFKKDSEKLP
PCKKPLSPVRDNIQLTPEAEEDIFNKPECGRVQRAIFQ.

### B. Inteins and Split-Inteins

It will be understood that in some embodiments (e.g., delivery of a prime editor in vivo), it may be advantageous to split a polypeptide (e.g., a reverse transcriptase or a napDNAbp) or a fusion protein (e.g., a prime editor) into an N-terminal half and a C-terminal half, deliver them separately, and then allow their colocalization to reform the complete protein (or fusion protein as the case may be) within the cell. Separate halves of a protein or a fusion protein may each comprise a split-intein tag to facilitate the reformation of the complete protein or fusion protein by the mechanism of protein trans splicing.

Protein trans-splicing, catalyzed by split inteins, provides an entirely enzymatic method for protein ligation. A split-intein is essentially a contiguous intein (e.g., a mini-intein) split into two pieces named N-intein and C-intein, respectively. The N-intein and C-intein of a split intein can associate non-covalently to form an active intein and catalyze the splicing reaction in essentially the same way as a contiguous intein does. Split inteins have been found in nature and have also been engineered in laboratories. As used herein, the term “split intein” refers to any intein in which one or more peptide bond breaks exists between the N-terminal and C-terminal amino acid sequences such that the N-terminal and C-terminal sequences become separate molecules that can non-covalently reassociate, or reconstitute, into an intein that is functional for trans-splicing reactions. Any catalytically active intein, or fragment thereof, may be used to derive a split intein for use in the methods of the invention. For example, in one aspect, the split intein may be derived from a eukaryotic intein. In another aspect, the split intein may be derived from a bacterial intein. In another aspect, the split intein may be derived from an archaeal intein. Preferably, the split intein so-derived will possess only the amino acid sequences essential for catalyzing trans-splicing reactions.

As used herein, the “N-terminal split intein (In)” refers to any intein sequence that comprises an N-terminal amino acid sequence that is functional for trans-splicing reactions. An In thus also comprises a sequence that is spliced out when trans-splicing occurs. An In can comprise a sequence that is a modification of the N-terminal portion of a naturally occurring intein sequence. For example, an In can comprise additional amino acid residues and/or mutated residues, as long as the inclusion of such additional and/or mutated residues does not render the In non-functional in trans-splicing. Preferably, the inclusion of the additional and/or mutated residues improves or enhances the trans-splicing activity of the In.

As used herein, the “C-terminal split intein (Ic)” refers to any intein sequence that comprises a C-terminal amino acid sequence that is functional for trans-splicing reactions. In one aspect, the Ic comprises 4 to 7 contiguous amino acid residues, at least 4 amino acids of which are from the last β-strand of the intein from which it was derived. An Ic thus also comprises a sequence that is spliced out when trans-splicing occurs. An Ic can comprise a sequence that is a modification of the C-terminal portion of a naturally occurring intein sequence. For example, an Ic can comprise additional amino acid residues and/or mutated residues, as long as the inclusion of such additional and/or mutated residues does not render the In non-functional in trans-splicing. Preferably, the inclusion of the additional and/or mutated residues improves or enhances the trans-splicing activity of the Ic.

In some embodiments of the invention, a peptide linked to an Ic or an In can comprise an additional chemical moiety including, among others, fluorescence groups, biotin, polyethylene glycol (PEG), amino acid analogs, unnatural amino acids, phosphate groups, glycosyl groups, radioisotope labels, and pharmaceutical molecules. In other embodiments, a peptide linked to an Ic can comprise one or more chemically reactive groups including, among others, ketones, aldehydes, Cys residues, and Lys residues. The N-intein and C-intein of a split intein can associate non-covalently to form an active intein and catalyze the splicing reaction when an “intein-splicing polypeptide (ISP)” is present. As used herein, “intein-splicing polypeptide (ISP)” refers to the portion of the amino acid sequence of a split intein that remains when the Ic, In, or both, are removed from the split intein. In certain embodiments, the In comprises the ISP. In another embodiment, the Ic comprises the ISP. In yet another embodiment, the ISP is a separate peptide that is not covalently linked to In nor to Ic.

Split inteins may be created from contiguous inteins by engineering one or more split sites in the unstructured loop or intervening amino acid sequence between the—12 conserved beta-strands found in the structure of mini-inteins. Some flexibility in the position of the split site within regions between the beta-strands may exist, provided that creation of the split will not disrupt the structure of the intein, the structured beta-strands in particular, to a sufficient degree that protein splicing activity is lost.

In protein trans-splicing, one precursor protein consists of an N-extein part followed by the N-intein, another precursor protein consists of the C-intein followed by a C-extein part, and a trans-splicing reaction (catalyzed by the N- and C-inteins together) excises the two intein sequences and links the two extein sequences with a peptide bond. Protein trans-splicing, being an enzymatic reaction, can work with very low (e.g., micromolar) concentrations of proteins and can be carried out under physiological conditions.

Exemplary sequences are as follows:

| NAME | SEQUENCE OF LIGAND-DEPENDENT INTEIN |
|---|---|
| 2-4 INTEIN: | CLAEGTRIFDPVTGTTHRIEDVVDGRKPIHVVAAAKDGTLLARPVV |
| | SWFDQGTRDVIGLRIAGGAIVWATPDHKVLTEYGWRAAGELRKGD |
| | RVAGPGGSGNSLALSLTADQMVSALLDAEPPILYSEYDPTSPFSEAS |
| | MMGLLTNLADRELVHMINWAKRVPGFVDLTLHDQAHLLECAWLEI |
| | LMIGLVWRSMEHPGKLLFAPNLLLDRNQGKCVEGMVEIFDMLLAT |
| | SSRFRMMNLQGEEFVCLKSIILLNSGVYTFLSSTLKSLEEKDHIHRA |
| | LDKITDTLIHLMAKAGLTLQQQHQRLAQLLLILSHIRHMSNKGMEH |
| | LYSMKYKNVVPLYDLLLEMLDAHRLHAGGSGASRVQAFADALDD |
| | KFLHDMLAEELRYSVIREVLPTRRARTFDLEVEELHTLVAEGVVVHNC |
| | (SEQ ID NO: 124) |
| 3-2 INTEIN | CLAEGTRIFDPVTGTTHRIEDVVDGRKPIHVVAVAKDGTLLARPVVS |
| | WFDQGTRDVIGLRIAGGAIVWATPDHKVLTEYGWRAAGELRKGDR |
| | VAGPGGSGNSLALSLTADQMVSALLDAEPPILYSEYDPTSPFSEASM |
| | MGLLTNLADRELVHMINWAKRVPGFVDLTLHDQAHLLECAWLEIL |
| | MIGLVWRSMEHPGKLLFAPNLLLDRNQGKCVEGMVEIFDMLLATS |
| | SRFRMMNLQGEEFVCLKSIILLNSGVYTFLSSTLKSLEEKDHIHRAL |
| | DKITDTLIHLMAKAGLTLQQQHQRLAQLLLILSHIRHMSNKGMEHL |
| | YSMKYTNVVPLYDLLLEMLDAHRLHAGGSGASRVQAFADALDDK |
| | FLHDMLAEELRYSVIREVLPTRRARTFDLEVEELHTLVAEGVVVHNC |
| | (SEQ ID NO: 125) |
| 30R3-1 INTEIN | CLAEGTRIFDPVTGTTHRIEDVVDGRKPIHVVAAAKDGTLLARPVV |
| | SWFDQGTRDVIGLRIAGGATVWATPDHKVLTEYGWRAAGELRKG |
| | DRVAGPGGSGNSLALSLTADQMVSALLDAEPPIPYSEYDPTSPFSEA |
| | SMMGLLTNLADRELVHMINWAKRVPGFVDLTLHDQAHLLECAWL |
| | EILMIGLVWRSMEHPGKLLFAPNLLLDRNQGKCVEGMVEIFDMLL |
| | ATSSRFRMMNLQGEEFVCLKSIILLNSGVYTFLSSTLKSLEEKDHIH |
| | RALDKITDTLIHLMAKAGLTLQQQHQRLAQLLLILSHIRHMSNKGM |
| | EHLYSMKYKNVVPLYDLLLEMLDAHRLHAGGSGASRVQAFADAL |
| | DDKFLHDMLAEGLRYSVIREVLPTRRARTFDLEVEELHTLVAEGVVVHNC |
| | (SEQ ID NO: 126) |
| 30R3-2 INTEIN | CLAEGTRIFDPVTGTTHRIEDVVDGRKPIHVVAAAKDGTLLARPVV |
| | SWFDQGTRDVIGLRIAGGATVWATPDHKVLTEYGWRAAGELRKG |
| | DRVAGPGGSGNSLALSLTADQMVSALLDAEPPILYSEYDPTSPFSEA |
| | SMMGLLTNLADRELVHMINWAKRVPGFVDLTLHDQAHLLECAWL |
| | EILMIGLVWRSMEHPGKLLFAPNLLLDRNQGKCVEGMVEIFDMLL |
| | ATSSRFRMMNLQGEEFVCLKSIILLNSGVYTFLSSTLKSLEEKDHIH |
| | RALDKITDTLIHLMAKAGLTLQQQHQRLAQLLLILSHIRHMSNKGM |
| | EHLYSMKYKNVVPLYDLLLEMLDAHRLHAGGSGASRVQAFADAL |
| | DDKFLHDMLAEELRYSVIREVLPTRRARTFDLEVEELHTLVAEGVVVHNC |
| | (SEQ ID NO: 127) |
| 30R3-3 INTEIN | CLAEGTRIFDPVTGTTHRIEDVVDGRKPIHVVAAAKDGTLLARPVV |
| | SWFDQGTRDVIGLRIAGGATVWATPDHKVLTEYGWRAAGELRKG |
| | DRVAGPGGSGNSLALSLTADQMVSALLDAEPPIPYSEYDPTSPFSEA |
| | SMMGLLTNLADRELVHMINWAKRVPGFVDLTLHDQAHLLECAWL |
| | EILMIGLVWRSMEHPGKLLFAPNLLLDRNQGKCVEGMVEIFDMLL |
| | ATSSRFRMMNLQGEEFVCLKSIILLNSGVYTFLSSTLKSLEEKDHIH |
| | RALDKITDTLIHLMAKAGLTLQQQHQRLAQLLLILSHIRHMSNKGM |
| | EHLYSMKYKNVVPLYDLLLEMLDAHRLHAGGSGASRVQAFADAL |
| | DDKFLHDMLAEELRYSVIREVLPTRRARTFDLEVEELHTLVAEGVVVHNC |
| | (SEQ ID NO: 128) |
| 37R3-1 INTEIN | CLAEGTRIFDPVTGTTHRIEDVVDGRKPIHVVAAAKDGTLLARPVV |
| | SWFDQGTRDVIGLRIAGGATVWATPDHKVLTEYGWRAAGELRKG |
| | DRVAGPGGSGNSLALSLTADQMVSALLDAEPPILYSEYNPTSPFSEA |
| | SMMGLLTNLADRELVHMINWAKRVPGFVDLTLHDQAHLLERAWL |
| | EILMIGLVWRSMEHPGKLLFAPNLLLDRNQGKCVEGMVEIFDMLL |
| | ATSSRFRMMNLQGEEFVCLKSIILLNSGVYTFLSSTLKSLEEKDHIH |
| | RALDKITDTLIHLMAKAGLTLQQQHQRLAQLLLILSHIRHMSNKGM |
| | EHLYSMKYKNVVPLYDLLLEMLDAHRLHAGGSGASRVQAFADAL |
| | DDKFLHDMLAEGLRYSVIREVLPTRRARTFDLEVEELHTLVAEGVVVHNC |
| | ((SEQ ID NO: 129) |
| 37R3-2 INTEIN | CLAEGTRIFDPVTGTTHRIEDVVDGRKPIHVVAAAKDGTLLARPVV |
| | SWFDQGTRDVIGLRIAGGAIVWATPDHKVLTEYGWRAAGELRKGD |
| | RVAGPGGSGNSLALSLTADQMVSALLDAEPPILYSEYDPTSPFSEAS |
| | MMGLLTNLADRELVHMINWAKRVPGFVDLTLHDQAHLLERAWLEI |
| | LMIGLVWRSMEHPGKLLFAPNLLLDRNQGKCVEGMVEIFDMLLAT |
| | SSRFRMMNLQGEEFVCLKSIILLNSGVYTFLSSTLKSLEEKDHIHRA |
| | LDKITDTLIHLMAKAGLTLQQQHQRLAQLLLILSHIRHMSNKGMEH |
| | LYSMKYKNVVPLYDLLLEMLDAHRLHAGGSGASRVQAFADALDD |
| | KFLHDMLAEGLRYSVIREVLPTRRARTFDLEVEELHTLVAEGVVVHNC |
| | (SEQ ID NO: 130) |
| 37R3-3 INTEIN | CLAEGTRIFDPVTGTTHRIEDVVDGRKPIHVVAVAKDGTLLARPVVS |
| | WFDQGTRDVIGLRIAGGATVWATPDHKVLTEYGWRAAGELRKGD |
| | RVAGPGGSGNSLALSLTADQMVSALLDAEPPILYSEYDPTSPFSEAS |
| | MMGLLTNLADRELVHMINWAKRVPGFVDLTLHDQAHLLERAWLEI |
| | LMIGLVWRSMEHPGKLLFAPNLLLDRNQGKCVEGMVEIFDMLLAT |
| | SSRFRMMNLQGEEFVCLKSIILLNSGVYTFLSSTLKSLEEKDHIHRA |
| | LDKITDTLIHLMAKAGLTLQQQHQRLAQLLLILSHIRHMSNKGMEH |
| | LYSMKYKNVVPLYDLLLEMLDAHRLHAGGSGASRVQAFADALDD |
| | KFLHDMLAEELRYSVIREVLPTRRARTFDLEVEELHTLVAEGVVVHNC |
| | (SEQ ID NO: 131) |

Although inteins are most frequently found as a contiguous domain, some exist in a naturally split form. In this case, the two fragments are expressed as separate polypeptides and must associate before splicing takes place, so-called protein trans-splicing.

An exemplary split intein is the Ssp DnaE intein, which comprises two subunits, namely, DnaE-N and DnaE-C. The two different subunits are encoded by separate genes, namely dnaE-n and dnaE-c, which encode the DnaE-N and DnaE-C subunits, respectively. DnaE is a naturally occurring split intein in Synechocytis sp. PCC6803 and is capable of directing trans-splicing of two separate proteins, each comprising a fusion with either DnaE-N or DnaE-C.

Additional naturally occurring or engineered split-intein sequences are known in the art or can be made from whole-intein sequences described herein or those available in the art. Examples of split-intein sequences can be found in Stevens et al., “A promiscuous split intein with expanded protein engineering applications,” PNAS, 2017, Vol. 114:8538-8543; Iwai et al., “Highly efficient protein trans-splicing by a naturally split DnaE intein from Nostc punctiforme, FEBS Lett, 580:1853-1858, each of which are incorporated herein by reference. Additional split intein sequences can be found, for example, in WO 2013/045632, WO 2014/055782, WO 2016/069774, and EP2877490, the contents of each of which are incorporated herein by reference.

In addition, protein splicing in trans has been described in vivo and in vitro (Shingledecker, et al., Gene 207:187 (1998), Southworth, et al., EMBO J. 17:918 (1998); Mills, et al., Proc. Natl. Acad. Sci. USA, 95:3543-3548 (1998); Lew, et al., J. Biol. Chem., 273:15887-15890 (1998); Wu, et al., Biochim. Biophys. Acta 35732:1 (1998b), Yamazaki, et al., J. Am. Chem. Soc. 120:5591 (1998), Evans, et al., J. Biol. Chem. 275:9091 (2000); Otomo, et al., Biochemistry 38:16040-16044 (1999); Otomo, et al., J. Biolmol. NMR 14:105-114 (1999); Scott, et al., Proc. Natl. Acad. Sci. USA 96:13638-13643 (1999)) and provides the opportunity to express a protein as two inactive fragments that subsequently undergo ligation to form a functional product.

### RNA-Protein Interaction Domain

In various embodiments, two separate protein domains (e.g., a Cas9 domain and a polymerase domain) may be colocalized to one another to form a functional complex (akin to the function of a fusion protein comprising the two separate protein domains) by using an “RNA-protein recruitment system,” such as the “MS2 tagging technique.” Such systems generally tag one protein domain with an “RNA-protein interaction domain” (a.k.a. “RNA-protein recruitment domain”) and the other with an “RNA-binding protein” that specifically recognizes and binds to the RNA-protein interaction domain, e.g., a specific hairpin structure. These types of systems can be leveraged to colocalize the domains of a prime editor, as well as to recruit additional functionalities to a prime editor, such as a UGI domain. In one example, the MS2 tagging technique is based on the natural interaction of the MS2 bacteriophage coat protein (“MCP” or “MS2cp”) with a stem-loop or hairpin structure present in the genome of the phage, i.e., the “MS2 hairpin.” In the case of the MS2 hairpin, it is recognized and bound by the MS2 bacteriophage coat protein (MCP). Thus, in one exemplary scenario, a reverse transcriptase-MS2 fusion can recruit a Cas9-MCP fusion.

A review of other modular RNA-protein interaction domains are described in the art, for example, in Johansson et al., “RNA recognition by the MS2 phage coat protein,” Sem Virol., 1997, Vol. 8(3): 176-185; Delebecque et al., “Organization of intracellular reactions with rationally designed RNA assemblies,” Science, 2011, Vol. 333:470-474; Mali et al., “Cas9 transcriptional activators for target specificity screening and paired nickases for cooperative genome engineering,” Nat. Biotechnol., 2013, Vol. 31:833-838; and Zalatan et al., “Engineering complex synthetic transcriptional programs with CRISPR RNA scaffolds,” Cell, 2015, Vol. 160:339-350, each of which are incorporated herein by reference in their entireties. Other systems include the PP7 hairpin, which specifically recruits the PCP protein, and the “com” hairpin, which specifically recruits the Com protein. See Zalatan et al.

The nucleotide sequence of the MS2 hairpin (or equivalently referred to as the “MS2 aptamer”) is: GCCAACATGAGGATCACCCATGTCTGCAGGGCC (SEQ ID NO: 144).

The amino acid sequence of the MCP or MS2cp is: GSASNFTQFVLVDNGGTGDVTVAPSNFANGVAEWISSNSRSQAYKVTCSVRQSSAQ NRKYTIKVEVPKVATQTVGGEELPVAGWRSYLNMELTIPIFATNSDCELIVKAMQGL LKDGNPIPSAIAANSGIY (SEQ ID NO: 145).

### C. Other PE Elements

In certain embodiments, the prime editors utilized in the methods and complexes described herein may comprise an inhibitor of base repair. The term “inhibitor of base repair” or “IBR” refers to a protein that is capable in inhibiting the activity of a nucleic acid repair enzyme, for example, a base excision repair enzyme. In some embodiments, the IBR is an inhibitor of OGG base excision repair. In some embodiments, the IBR is an inhibitor of base excision repair (“iBER”). Exemplary inhibitors of base excision repair include inhibitors of APE1, Endo III, Endo IV, Endo V, Endo VIII, Fpg, hOGG1, hNEIL1, T7 EndoI, T4PDG, UDG, hSMUG1, and hAAG. In some embodiments, the IBR is an inhibitor of Endo V or hAAG. In some embodiments, the IBR is an iBER that may be a catalytically inactive glycosylase or catalytically inactive dioxygenase or a small molecule or peptide inhibitor of an oxidase, or variants thereof. In some embodiments, the IBR is an iBER that may be a TDG inhibitor, an MBD4 inhibitor, or an inhibitor of an AlkBH enzyme. In some embodiments, the IBR is an iBER that comprises a catalytically inactive TDG or catalytically inactive MBD4. An exemplary catalytically inactive TDG is an N140A mutant of SEQ ID NO: 136 (human TDG).

Some exemplary glycosylases are provided below. The catalytically inactivated variants of any of these glycosylase domains are iBERs that may be fused to the napDNAbp or polymerase domain of the prime editors utilized in the methods and compositions provided in this disclosure.

OGG (human)
(SEQ ID NO: 133)
MPARALLPRRMGHRTLASTPALWASIPCPRSELRLDLVLPSGQSFRWRE
QSPAHWSGVLADQVWTLTQTEEQLHCTVYRGDKSQASRPTPDELEAVRK
YFQLDVTLAQLYHHWGSVDSHFQEVAQKFQGVRLLRQDPIECLFSFICS
SNNNIARITGMVERLCQAFGPRLIQLDDVTYHGFPSLQALAGPEVEAHL
RKLGLGYRARYVSASARAILEEQGGLAWLQQLRESSYEEAHKALCILPG
VGTKVADCICLMALDKPQAVPVDVHMWHIAQRDYSWHPTTSQAKGPSPQ
TNKELGNFFRSLWGPYAGWAQAVLFSADLRQSRHAQEPPAKRRKGSKG
PEG
MPG (human)
(SEQ ID NO: 134)
MVTPALQMKKPKQFCRRMGQKKQRPARAGQPHSSSDAAQAPAEQPHSS
SDAAQAPCPRERCLGPPTTPGPYRSIYFSSPKGHLTRLGLEFFDQPAV
PLARAFLGQVLVRRLPNGTELRGRIVETEAYLGPEDEAAHSRGGRQTP
RNRGMFMKPGTLYVYIIYGMYFCMNISSQGDGACVLLRALEPLEGLET
MRQLRSTLRKGTASRVLKDRELCSGPSKLCQALAINKSFDQRDLAQDE
AVWLERGPLEPSEPAVVAAARVGVGHAGEWARKPLRFYVRGSPWVSVV
DRVAEQDTQA
MBD4 (human)
(SEQ ID NO: 135)
MGTTGLESLSLGDRGAAPTVTSSERLVPDPPNDLRKEDVAMELERVGE
DEEQMMIKRSSECNPLLQEPIASAQFGATAGTECRKSVPCGWERVVKQ
RLFGKTAGRFDVYFISPQGLKFRSKSSLANYLHKNGETSLKPEDFDFT
VLSKRGIKSRYKDCSMAALTSHLQNQSNNSNWNLRTRSKCKKDVFMPP
SSSSELQESRGLSNFTSTHLLLKEDEGVDDVNFRKVRKPKGKVTILKG
IPIKKTKKGCRKSCSGFVQSDSKRESVCNKADAESEPVAQKSQLDRTV
CISDAGACGETLSVTSEENSLVKKKERSLSSGSNFCSEQKTSGIINKF
CSAKDSEHNEKYEDTFLESEEIGTKVEVVERKEHLHTDILKRGSEMDN
NCSPTRKDFTGEKIFQEDTIPRTQIERRKTSLYFSSKYNKEALSPPRR
KAFKKWTPPRSPFNLVQETLFHDPWKLLIATIFLNRTSGKMAIPVLWK
FLEKYPSAEVARTADWRDVSELLKPLGLYDLRAKTIVKFSDEYLTKQW
KYPIELHGIGKYGNDSYRIFCVNEWKQVHPEDHKLNKYHDWLWENHEK
LSLS
TDG (human)
(SEQ ID NO: 136)
MEAENAGSYSLQQAQAFYTFPFQQLMAEAPNMAVVNEQQMPEEVPAPA
PAQEPVQEAPKGRKRKPRTTEPKQPVEPKKPVESKKSGKSAKSKEKQE
KITDTFKVKRKVDRENGVSEAELLTKTLPDILTFNLDIVIIGINPGLM
AAYKGHHYPGPGNHFWKCLFMSGLSEVQLNHMDDHTLPGKYGIGFTNM
VERTTPGSKDLSSKEFREGGRILVQKLQKYQPRIAVFNGKCIYEIFSK
EVFGVKVKNLEFGLQPHKIPDTETLCYVMPSSSARCAQFPRAQDKVHY
YIKLKDLRDQLKGIERNMDVQEVQYTFDLQLAQEDAKKMAVKEEKYDP
GYEAAYGGAYGENPCSSEPCGESSNGLIESVELRGESAFSGIPNGQWM
TQSFTDQIPSFSNHCGTQEQEEESHA

In some embodiments, the fusion proteins described herein may comprise one or more heterologous protein domains (e.g., about or more than about 1, 2, 3, 4, 5, 6, 7, 8, 9, 10, or more domains in addition to the prime editor components). A fusion protein may comprise any additional protein sequence, and optionally a linker sequence between any two domains. Other exemplary features that may be present are localization sequences, such as cytoplasmic localization sequences, export sequences, such as nuclear export sequences, or other localization sequences, as well as sequence tags that are useful for solubilization, purification, or detection of the fusion proteins.

Examples of protein domains that may be fused to a prime editor or component thereof (e.g., the napDNAbp domain, the polymerase domain, or the NLS domain) include, without limitation, epitope tags and reporter gene sequences. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5-transferase (GST), horseradish peroxidase (HRP), chloramphenicol acetyltransferase (CAT), beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). A prime editor may be fused to a gene sequence encoding a protein or a fragment of a protein that binds DNA molecules or binds other cellular molecules, including, but not limited to, maltose binding protein (MBP), S-tag, Lex A DNA binding domain (DBD) fusions, GAL4 DNA binding domain fusions, and herpes simplex virus (HSV) BP16 protein fusions. Additional domains that may form part of a prime editor are described in US Patent Publication No. 2011/0059502, published Mar. 10, 2011, and incorporated herein by reference.

In an aspect of the disclosure, a reporter gene that includes, but is not limited to, glutathione-5-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP), may be introduced into a cell to encode a gene product that serves as a marker by which to measure the alteration or modification of expression of the gene product. In certain embodiments of the disclosure, the gene product is luciferase. In a further embodiment of the disclosure, the expression of the gene product is decreased.

Suitable protein tags provided herein include, but are not limited to, biotin carboxylase carrier protein (BCCP) tags, myc-tags, calmodulin-tags, FLAG-tags, hemagglutinin (HA)-tags, polyhistidine tags, also referred to as histidine tags or His-tags, maltose binding protein (MBP)-tags, nus-tags, glutathione-S-transferase (GST)-tags, green fluorescent protein (GFP)-tags, thioredoxin-tags, S-tags, Softags (e.g., Softag 1, Softag 3), strep-tags, biotin ligase tags, FLASH tags, V5 tags, and SBP-tags. Additional suitable sequences will be apparent to those of skill in the art. In some embodiments, the fusion protein comprises one or more His tags.

In some embodiments of the present disclosure, the activity of the prime editing system may be temporally regulated by adjusting the residence time, the amount, and/or the activity of the expressed components of the PE system. For example, as described herein, the PE may be fused with a protein domain that is capable of modifying the intracellular half-life of the PE. In certain embodiments involving two or more vectors (e.g., a vector system in which the components described herein are encoded on two or more separate vectors), the activity of the PE system may be temporally regulated by controlling the timing in which the vectors are delivered. For example, in some embodiments, a vector encoding the nuclease system may deliver the PE prior to the vector encoding the template. In other embodiments, the vector encoding the PEgRNA may deliver the guide prior to the vector encoding the PE system. In some embodiments, the vectors encoding the PE system and PEgRNA are delivered simultaneously. In certain embodiments, the simultaneously delivered vectors temporally deliver, e.g., the PE, PEgRNA, and/or second strand guide RNA components. In further embodiments, the RNA (such as, e.g., the nuclease transcript) transcribed from the coding sequence on the vectors may further comprise at least one element that is capable of modifying the intracellular half-life of the RNA and/or modulating translational control. In some embodiments, the half-life of the RNA may be increased. In some embodiments, the half-life of the RNA may be decreased. In some embodiments, the element may be capable of increasing the stability of the RNA. In some embodiments, the element may be capable of decreasing the stability of the RNA. In some embodiments, the element may be within the 3′ UTR of the RNA. In some embodiments, the element may include a polyadenylation signal (PA). In some embodiments, the element may include a cap, e.g., an upstream mRNA or PEgRNA end. In some embodiments, the RNA may comprise no PA such that it is subject to quicker degradation in the cell after transcription. In some embodiments, the element may include at least one AU-rich element (ARE). The AREs may be bound by ARE binding proteins (ARE-BPs) in a manner that is dependent upon tissue type, cell type, timing, cellular localization, and environment. In some embodiments, the destabilizing element may promote RNA decay, affect RNA stability, or activate translation. In some embodiments, the ARE may comprise 50 to 150 nucleotides in length. In some embodiments, the ARE may comprise at least one copy of the sequence AUUUA. In some embodiments, at least one ARE may be added to the 3′ UTR of the RNA. In some embodiments, the element may be a Woodchuck Hepatitis Virus (WHP).

Posttranscriptional Regulatory Element (WPRE), which creates a tertiary structure to enhance expression from the transcript. In further embodiments, the element is a modified and/or truncated WPRE sequence that is capable of enhancing expression from the transcript, as described, for example in Zufferey et al., J. Virol., 73(4): 2886-92 (1999) and Flajolet et al., J. Virol., 72(7): 6175-80 (1998). In some embodiments, the WPRE or equivalent may be added to the 3′ UTR of the RNA. In some embodiments, the element may be selected from other RNA sequence motifs that are enriched in either fast- or slow-decaying transcripts.

In some embodiments, the vector encoding the PE or the PEgRNA may be self-destroyed via cleavage of a target sequence present on the vector by the PE system. The cleavage may prevent continued transcription of a PE or a PEgRNA from the vector. Although transcription may occur on the linearized vector for some amount of time, the expressed transcripts or proteins subject to intracellular degradation will have less time to produce off-target effects without continued supply from expression of the encoding vectors.

### Pharmaceutical Compositions

Other aspects of the present disclosure relate to pharmaceutical compositions comprising any of the guide RNAs (including, e.g., PEgRNAs, ePEgRNAs, and second strand nicking gRNAs), fusion proteins, and polynucleotides described herein. The term “pharmaceutical composition,” as used herein, refers to a composition formulated for pharmaceutical use. In some embodiments, the pharmaceutical composition further comprises a pharmaceutically acceptable carrier. In some embodiments, the pharmaceutical composition comprises additional agents (e.g., for specific delivery, increasing half-life, or other therapeutic compounds).

As used herein, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the compound from one site (e.g., the delivery site) of the body, to another site (e.g., an organ, tissue, or other part of the body). A pharmaceutically acceptable carrier is “acceptable” in the sense of being compatible with the other ingredients of the formulation and not injurious to the tissue of the subject (e.g., physiologically compatible, sterile, physiologic pH, etc.). Some examples of materials that can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil, and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids; (23) serum component, such as serum albumin, HDL, and LDL; (22) C2-C12 alcohols, such as ethanol; and (23) other non-toxic compatible substances employed in pharmaceutical formulations. Wetting agents, coloring agents, release agents, coating agents, sweetening agents, flavoring agents, perfuming agents, preservatives, and antioxidants can also be present in the formulation. The terms such as “excipient,” “carrier,” “pharmaceutically acceptable carrier,” or the like are used interchangeably herein.

In some embodiments, the pharmaceutical composition is formulated for delivery to a subject, e.g., for gene editing. Suitable routes of administering the pharmaceutical composition described herein include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration.

In some embodiments, the pharmaceutical composition described herein is administered locally to a diseased site (e.g., tumor site). In some embodiments, the pharmaceutical composition described herein is administered to a subject by injection, by means of a catheter, by means of a suppository, or by means of an implant, the implant being of a porous, non-porous, or gelatinous material, including a membrane, such as a sialastic membrane, or a fiber.

In other embodiments, the pharmaceutical composition described herein is delivered in a controlled release system. In one embodiment, a pump may be used (see, e.g., Langer, 1990, Science 249:1527-1533; Sefton, 1989, CRC Crit. Ref. Biomed. Eng. 14:201; Buchwald et al., 1980, Surgery 88:507; Saudek et al., 1989, N. Engl. J. Med. 321:574). In another embodiment, polymeric materials can be used. (See, e.g., Medical Applications of Controlled Release (Langer and Wise eds., CRC Press, Boca Raton, Fla., 1974); Controlled Drug Bioavailability, Drug Product Design and Performance (Smolen and Ball eds., Wiley, New York, 1984); Ranger and Peppas, 1983, Macromol. Sci. Rev. Macromol. Chem. 23:61. See also Levy et al., 1985, Science 228:190; During et al., 1989, Ann. Neurol. 25:351; Howard et al., 1989, J. Neurosurg. 71:105). Other controlled release systems are discussed, for example, in Langer, supra.

In some embodiments, the pharmaceutical composition is formulated in accordance with routine procedures as a composition adapted for intravenous or subcutaneous administration to a subject, e.g., a human. In some embodiments, pharmaceutical compositions for administration by injection are solutions in sterile isotonic aqueous buffer. Where necessary, the pharmaceutical composition can also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the pharmaceutical composition is to be administered by infusion, it can be dispensed with an infusion bottle containing sterile pharmaceutical grade water or saline. Where the pharmaceutical composition is administered by injection, an ampoule of sterile water for injection or saline can be provided so that the ingredients can be mixed prior to administration.

A pharmaceutical composition for systemic administration may be a liquid, e.g., sterile saline, lactated Ringer's or Hank's solution. In addition, the pharmaceutical composition can be in solid forms and re-dissolved or suspended immediately prior to use. Lyophilized forms are also contemplated.

The pharmaceutical composition can be contained within a lipid particle or vesicle, such as a liposome or microcrystal, which is also suitable for parenteral administration. The particles can be of any suitable structure, such as unilamellar or plurilamellar, so long as compositions are contained therein. Compounds can be entrapped in “stabilized plasmid-lipid particles” (SPLP) containing the fusogenic lipid dioleoylphosphatidylethanolamine (DOPE), low levels (5-10 mol %) of cationic lipid and stabilized by a polyethyleneglycol (PEG) coating (Zhang Y. P. et al., Gene Ther. 1999, 6:1438-47). Positively charged lipids such as N-[1-(2,3-dioleoyloxi)propyl]-N,N,N-trimethyl-amoniummethylsulfate, or “DOTAP,” are particularly preferred for such particles and vesicles. The preparation of such lipid particles is well known. See, e.g., U.S. Pat. Nos. 4,880,635; 4,906,477; 4,911,928; 4,917,951; 4,920,016; and 4,921,757; each of which is incorporated herein by reference.

The pharmaceutical compositions described herein may be administered or packaged as a unit dose, for example. The term “unit dose” when used in reference to a pharmaceutical composition of the present disclosure refers to physically discrete units suitable as unitary dosage for the subject, each unit containing a predetermined quantity of active material calculated to produce the desired therapeutic effect in association with the required diluent; i.e., carrier, or vehicle.

Further, the pharmaceutical composition can be provided as a pharmaceutical kit comprising (a) a container containing a compound of the invention in lyophilized form and (b) a second container containing a pharmaceutically acceptable diluent (e.g., sterile water) for injection. The pharmaceutically acceptable diluent can be used for reconstitution or dilution of the lyophilized compound of the invention. Optionally associated with such container(s) can be a notice in the form prescribed by a governmental agency regulating the manufacture, use, or sale of pharmaceuticals or biological products, which notice reflects approval by the agency of manufacture, use, or sale for human administration.

In another aspect, an article of manufacture containing materials useful for the treatment of the diseases described above is included. In some embodiments, the article of manufacture comprises a container and a label. Suitable containers include, for example, bottles, vials, syringes, and test tubes. The containers may be formed from a variety of materials such as glass or plastic. In some embodiments, the container holds a composition that is effective for treating a disease and may have a sterile access port. For example, the container may be an intravenous solution bag or a vial having a stopper pierce-able by a hypodermic injection needle. The active agent in the composition is a compound of the invention. In some embodiments, the label on or associated with the container indicates that the composition is used for treating the disease of choice. The article of manufacture may further comprise a second container comprising a pharmaceutically acceptable buffer, such as phosphate-buffered saline, Ringer's solution, or dextrose solution. It may further include other materials desirable from a commercial and user standpoint, including other buffers, diluents, filters, needles, syringes, and package inserts with instructions for use.

### Kits and Cells

The guide RNAs (including pegRNAs and epegRNAs), fusion proteins, and compositions of the present disclosure may be assembled into kits. In some embodiments, the kit comprises polynucleotides for expression of the prime editors and/or pegRNAs, epegRNAs, and second strand nicking gRNAs described herein. In other embodiments, the kit further comprises appropriate guide nucleotide sequences or nucleic acid vectors for the expression of such guide nucleotide sequences, to target the Cas9 protein of the prime editors to the desired target sequence (e.g., a gene associate with a triplet repeat disorder, such as the HTT or FXN genes).

The kits described herein may include one or more containers housing components for performing the methods described herein, and optionally instructions for use. Any of the kits described herein may further comprise components needed for performing the prime editing methods described herein. Each component of the kits, where applicable, may be provided in liquid form (e.g., in solution) or in solid form, (e.g., a dry powder). In certain cases, some of the components may be reconstitutable or otherwise processible (e.g., to an active form), for example, by the addition of a suitable solvent or other species (for example, water), which may or may not be provided with the kit.

In some embodiments, the kits may optionally include instructions and/or promotion for use of the components provided. As used herein, “instructions” can define a component of instruction and/or promotion, and typically involve written instructions on or associated with packaging of the disclosure. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. The written instructions may be in a form prescribed by a governmental agency regulating the manufacture, use, or sale of pharmaceuticals or biological products, which can also reflect approval by the agency of manufacture, use, or sale for animal administration. As used herein, “promoted” includes all methods of doing business including methods of education, hospital and other clinical instruction, scientific inquiry, drug discovery or development, academic research, pharmaceutical industry activity including pharmaceutical sales, and any advertising or other promotional activity including written, oral, and electronic communication of any form, associated with the disclosure. Additionally, the kits may include other components depending on the specific application, as described herein.

The kits may contain any one or more of the components described herein in one or more containers. The components may be prepared sterilely, packaged in a syringe, and shipped refrigerated. Alternatively, they may be housed in a vial or other container for storage. A second container may have other components prepared sterilely. Alternatively, the kits may include the active agents premixed and shipped in a vial, tube, or other container.

The kits may have a variety of forms, such as a blister pouch, a shrink-wrapped pouch, a vacuum sealable pouch, a sealable thermoformed tray, or a similar pouch or tray form, with the accessories loosely packed within the pouch, one or more tubes, containers, a box, or a bag. The kits may be sterilized after the accessories are added, thereby allowing the individual accessories in the container to be otherwise unwrapped. The kits can be sterilized using any appropriate sterilization techniques, such as radiation sterilization, heat sterilization, or other sterilization methods known in the art. The kits may also include other components, depending on the specific application, for example, containers, cell media, salts, buffers, reagents, syringes, needles, a fabric, such as gauze, for applying or removing a disinfecting agent, disposable gloves, a support for the agents prior to administration, etc. Some aspects of this disclosure provide kits comprising a nucleic acid construct comprising a nucleotide sequence encoding the prime editor systems described herein, or various components thereof (e.g., including, but not limited to, the napDNAbps, reverse transcriptase domains, and pegRNAs/epegRNAs/second strand nicking gRNAs). In some embodiments, the nucleotide sequence(s) comprises a heterologous promoter (or more than a single promoter) that drives expression of the prime editor system components.

Other aspects of this disclosure provide kits comprising one or more nucleic acid constructs encoding the various components of the prime editing system described herein. In some embodiments, the nucleotide sequence comprises a heterologous promoter that drives expression of the prime editing system components.

Cells that may contain any of the guide RNAs, fusion proteins, and compositions described herein include prokaryotic cells and eukaryotic cells. The methods described herein may be used to deliver a prime editor and/or guide RNA into a eukaryotic cell (e.g., a mammalian cell, such as a human cell). In some embodiments, the cell is in vitro (e.g., a cultured cell). In some embodiments, the cell is in vivo (e.g., in a subject, such as a human subject). In some embodiments, the cell is ex vivo (e.g., isolated from a subject and may be administered back to the same or a different subject).

Mammalian cells of the present disclosure include human cells, primate cells (e.g., vero cells), rat cells (e.g., GH3 cells, OC23 cells) or mouse cells (e.g., MC3T3 cells). There are a variety of human cell lines, including, without limitation, human embryonic kidney (HEK) cells, HeLa cells, cancer cells from the National Cancer Institute's 60 cancer cell lines (NCI60), DU145 (prostate cancer) cells, Lncap (prostate cancer) cells, MCF-7 (breast cancer) cells, MDA-MB-438 (breast cancer) cells, PC3 (prostate cancer) cells, T47D (breast cancer) cells, THP-1 (acute myeloid leukemia) cells, U87 (glioblastoma) cells, SHSY5Y human neuroblastoma cells (cloned from a myeloma) and Saos-2 (bone cancer) cells. In some embodiments, prime editors and/or guide RNAs are delivered into human embryonic kidney (HEK) cells (e.g., HEK293 or HEK293T cells). In some embodiments, prime editors and/or guide RNAs are delivered into stem cells (e.g., human stem cells), such as, for example, pluripotent stem cells (e.g., human pluripotent stem cells including human induced pluripotent stem cells (hiPSCs)). A stem cell refers to a cell with the ability to divide for indefinite periods in culture and to give rise to specialized cells. A pluripotent stem cell refers to a type of stem cell that is capable of differentiating into all tissues of an organism, but not alone capable of sustaining full organismal development. A human induced pluripotent stem cell refers to a somatic (e.g., mature or adult) cell that has been reprogrammed to an embryonic stem cell-like state by being forced to express genes and factors important for maintaining the defining properties of embryonic stem cells (see, e.g., Takahashi and Yamanaka, Cell 126 (4): 663-76, 2006, incorporated by reference herein). Human induced pluripotent stem cells express stem cell markers and are capable of generating cells characteristic of all three germ layers (ectoderm, endoderm, mesoderm).

Additional non-limiting examples of cell lines that may be used in accordance with the present disclosure include 293-T, 293-T, 3T3, 4T1, 721, 9L, A-549, A172, A20, A253, A2780, A2780ADR, A2780cis, A431, ALC, B16, B35, BCP-1, BEAS-2B, bEnd.3, BHK-21, BR 293, BxPC3, C2C12, C3H-10T1/2, C6, C6/36, Cal-27, CGR8, CHO, CML T1, CMT, COR-L23, COR-L23/5010, COR-L23/CPR, COR-L23/R23, COS-7, COV-434, CT26, D17, DH82, DU145, DuCaP, E14Tg2a, EL4, EM2, EM3, EMT6/AR1, EMT6/AR10.0, FM3, H1299, H69, HB54, HB55, HCA2, Hepa1c1c7, High Five cells, HL-60, HMEC, HT-29, HUVEC, J558L cells, Jurkat, JY cells, K562 cells, KCL22, KG1, Ku812, KYO1, LNCap, Ma-Mel 1, 2, 3 . . . 48, MC-38, MCF-10A, MCF-7, MDA-MB-231, MDA-MB-435, MDA-MB-468, MDCK II, MG63, MONO-MAC 6, MOR/0.2R, MRC5, MTD-1A, MyEnd, NALM-1, NCI-H69/CPR, NCI-H69/LX10, NCI-H69/LX20, NCI-H69/LX4, NIH-3T3, NW-145, OPCN/OPCT Peer, PNT-1A/PNT 2, PTK2, Raji, RBL cells, RenCa, RIN-5F, RMA/RMAS, S2, Saos-2 cells, Sf21, Sf9, SiHa, SKBR3, SKOV-3, T-47D, T2, T84, THP1, U373, U87, U937, VCaP, WM39, WT-49, X63, YAC-1, and YAR cells.

Some aspects of this disclosure provide cells comprising any of the constructs disclosed herein. In some embodiments, a host cell is transiently or non-transiently transfected with one or more vectors described herein. In some embodiments, a cell is transfected as it naturally occurs in a subject. In some embodiments, a cell that is transfected is taken from a subject. In some embodiments, the cell is derived from cells taken from a subject, such as a cell line. A wide variety of cell lines for tissue culture are known in the art. Examples of cell lines include, but are not limited to, C8161, CCRF-CEM, MOLT, mIMCD-3, NHDF, HeLa-S3, Huh1, Huh4, Huh7, HUVEC, HASMC, HEKn, HEKa, MiaPaCell, Panc1, PC-3, TF1, CTLL-2, C1R, Rat6, CV1, RPTE, A10, T24, J82, A375, ARH-77, Calu1, SW480, SW620, SKOV3, SK-UT, CaCo2, P388D1, SEM-K2, WEHI-231, HB56, TIB55, Jurkat, J45.01, LRMB, Bcl-1, BC-3, IC21, DLD2, Raw264.7, NRK, NRK-52E, MRC5, MEF, Hep G2, HeLa B, HeLa T4, COS, COS-1, COS-6, COS-M6A, BS-C-1 monkey kidney epithelial, BALB/3T3 mouse embryo fibroblast, 3T3 Swiss, 3T3-L1, 132-d5 human fetal fibroblasts; 10.1 mouse fibroblasts, 293-T, 3T3, 721, 9L, A2780, A2780ADR, A2780cis, A 172, A20, A253, A431, A-549, ALC, B16, B35, BCP-1 cells, BEAS-2B, bEnd.3, BHK-21, BR 293, BxPC3, C3H-10T1/2, C6/36, Cal-27, CHO, CHO-7, CHO-IR, CHO-K1, CHO-K2, CHO-T, CHO Dhfr−/−, COR-L23, COR-L23/CPR, COR-L23/5010, COR-L23/R23, COS-7, COV-434, CML T1, CMT, CT26, D17, DH82, DU145, DuCaP, EL4, EM2, EM3, EMT6/AR1, EMT6/AR10.0, FM3, H1299, H69, HB54, HB55, HCA2, HEK-293, HeLa, Hepa1c1c7, HL-60, HMEC, HT-29, Jurkat, JY cells, K562 cells, Ku812, KCL22, KG1, KYO1, LNCap, Ma-Mel 1-48, MC-38, MCF-7, MCF-10A, MDA-MB-231, MDA-MB-468, MDA-MB-435, MDCK II, MDCK 11, MOR/0.2R, MONO-MAC 6, MTD-1A, MyEnd, NCI-H69/CPR, NCI-H69/LX10, NCI-H69/LX20, NCI-H69/LX4, NIH-3T3, NALM-1, NW-145, OPCN/OPCT cell lines, Peer, PNT-1A/PNT 2, RenCa, RIN-5F, RMA/RMAS, Saos-2 cells, Sf-9, SkBr3, T2, T-47D, T84, THP1 cell line, U373, U87, U937, VCaP, Vero cells, WM39, WT-49, X63, YAC-1, YAR, and transgenic varieties thereof.

Cell lines are available from a variety of sources known to those with skill in the art (see, e.g., the American Type Culture Collection (ATCC) (Manassas, VA)). In some embodiments, a cell transfected with one or more vectors described herein is used to establish a new cell line comprising one or more vector-derived sequences. In some embodiments, a cell transiently transfected with the components of a CRISPR system as described herein (such as by transient transfection of one or more vectors, or transfection with RNA), and modified through the activity of a CRISPR complex, is used to establish a new cell line comprising cells containing the modification but lacking any other exogenous sequence. In some embodiments, cells transiently or non-transiently transfected with one or more vectors described herein, or cell lines derived from such cells, are used in assessing one or more test compounds.

### Examples

Example 1. Using Prime Editing to Reduce the Size of CAG Repeat Tracts to a Normal polyQ Length

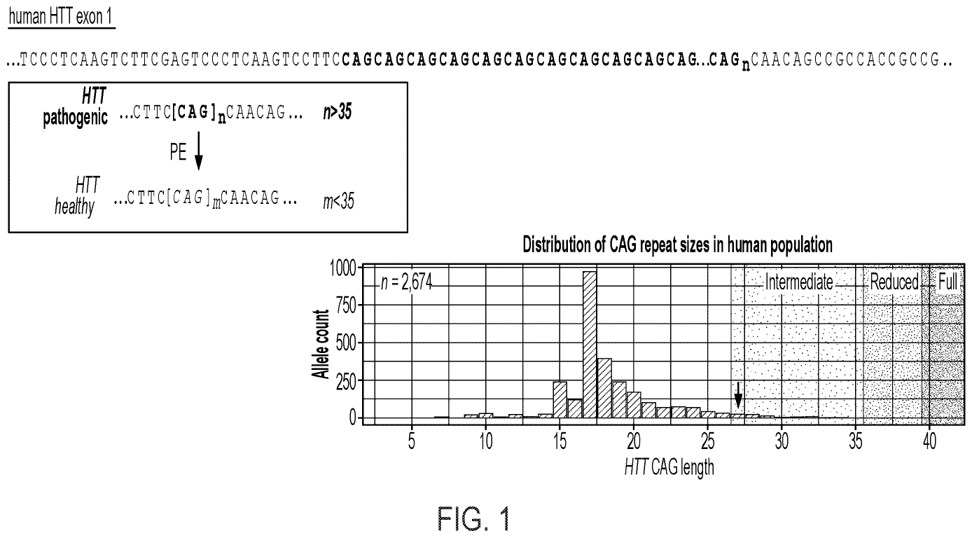

In Huntington's disease (HD), the length of CAG repeats in HTT are typically ≥40, while repeat length in the general population is described as a normal distribution ranging from 9 to 35 CAGs, with a median of 17 CAGs^{14,15} (FIG. 1). PolyQ lengths in HTT below this normal range have not been observed, and extreme truncation of polyQ is considered detrimental to HTT protein function¹⁰. Excision of the locus surrounding the CAG repeats in HTT exon 1 using paired nucleases has been demonstrated to alleviate polyQ proteinopathy in vitro and improve motor deficits in mice¹⁶⁻¹⁹. However, these strategies result in the additional loss of coding sequence and also cannot differentiate between pathogenic and non-pathogenic HTT alleles, thus resulting in loss of wild type HTT protein expression. ^{10, 11, 16-18, 20} The outcome of HTT loss in cells is not fully known^{21,22}. Recent clinical trial outcomes, however, suggest that loss of HTT expression may in fact induce toxicity in HD²³.

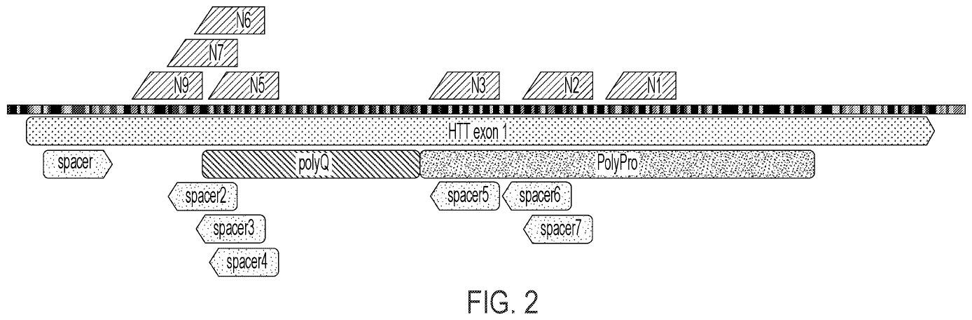

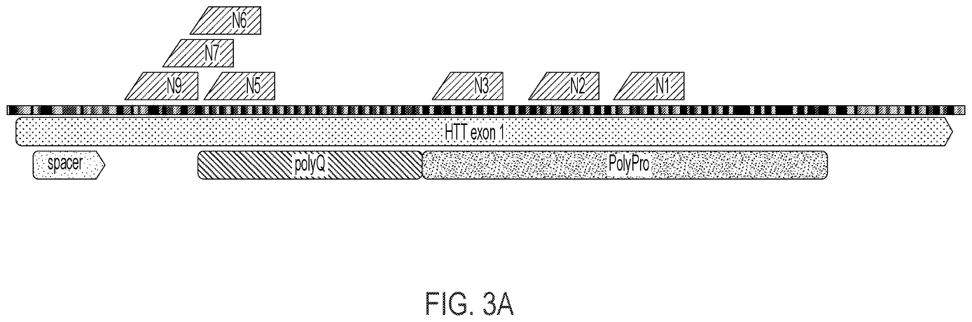

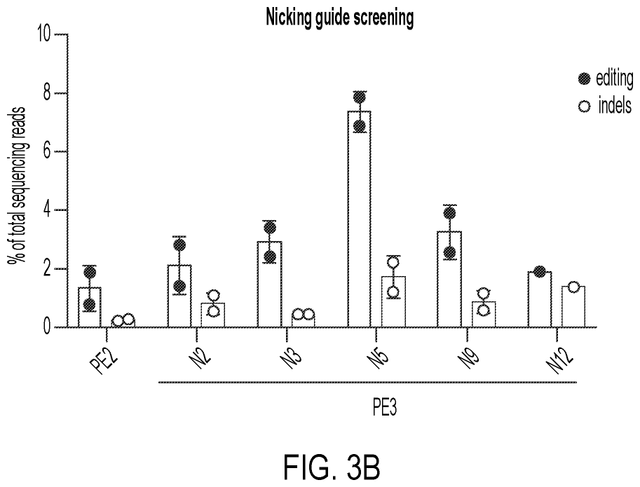

Prime editing¹² was explored as a means to precisely modify HTT exon 1 to replace CAG repeats with a small, normal number of triplets without disruption of the surrounding sequence. A few hundred prime editing strategies (PE2) were extensively screened using various prime editing sgRNA (pegRNA) designs. Multiple spacer sequences paired with different PBS and RT template (RTT: homology+synthesis template sequence) lengths were tested, which allowed selection of the best spacer sequence (“spacer”) enabling up to 2-4% average replacement of CAG repeats at endogenous loci in HEK293T cells (FIGS. 2 and 3A-3B). To increase editing efficiency, PE3 strategies, which pair PE2 editing with an additional sgRNA to induce a nearby nick near or within the editing target site to bias DNA repair towards incorporation of the prime edit^{12,24,25 3}, were explored. Out of five different nicking conditions, one was selected—nick N5—that resulted in the highest editing and the best editing: indel ratio (FIGS. 3A-3B).

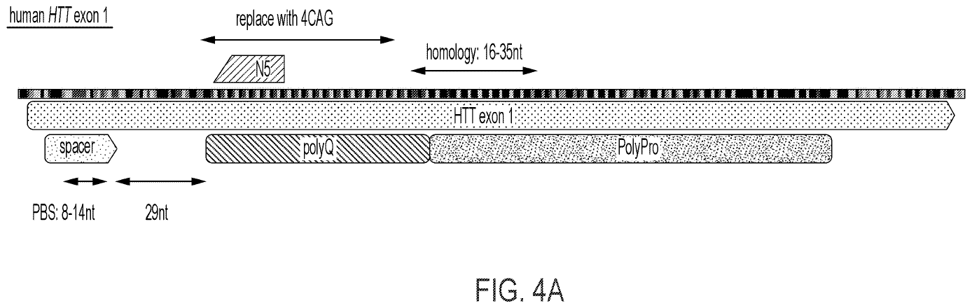

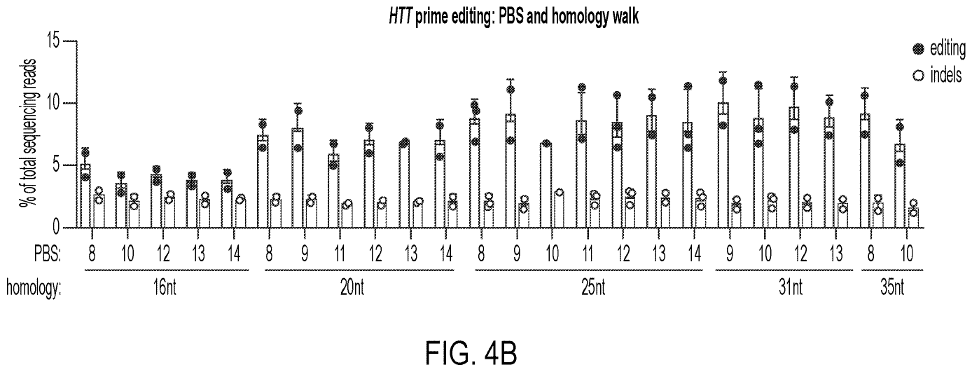

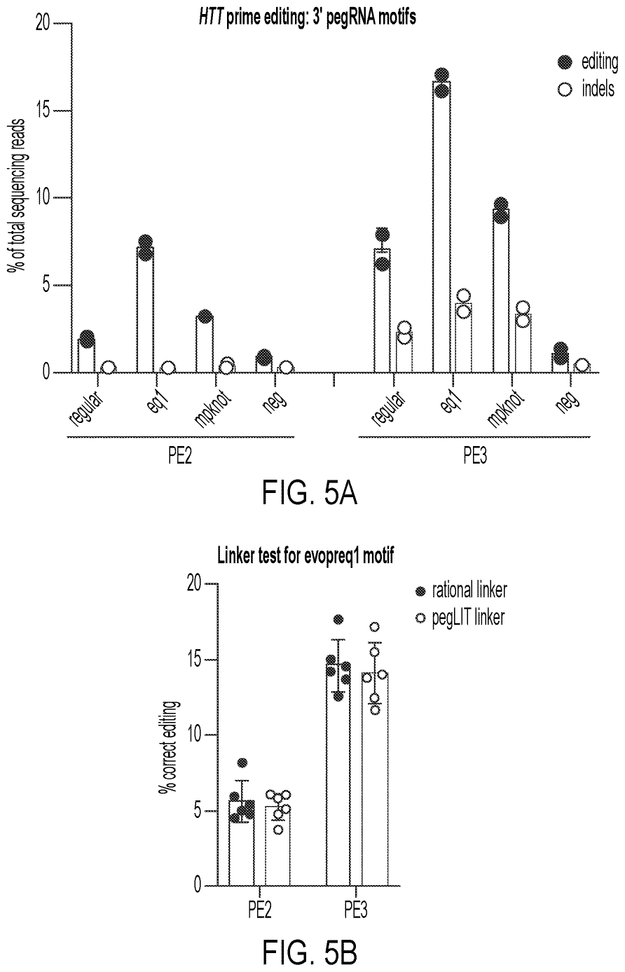

Next, another extensive screen of pegRNA designs with varying PBS and RTT lengths was performed in a PE3 system with an extra N5 nick. From all tested conditions, pegRNAs with PBS greater than 8 nt and homology longer than 25 nt resulted in the highest prime editing efficiency (FIGS. 4A-4B). Next, additional prime editing improvements at this locus were further evaluated. pegRNA with 3′ structural motifs (evopreq1 and mpknot)²⁶ were tested with rationally designed or computed by pegLIT²⁶ linker sequences, and an ~2-fold increase in editing efficiency was observed after applying evopreq1 motif with a rationally designed linker sequence (FIGS. 5A-5B). All designs moving forward include the evopreq1 motif and are referred to as “epegRNA”.

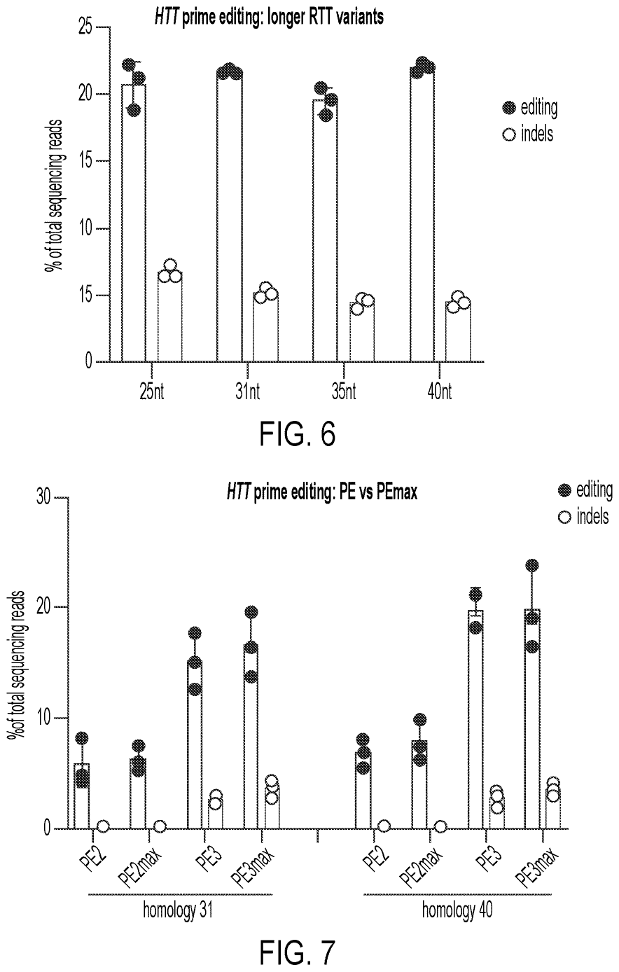

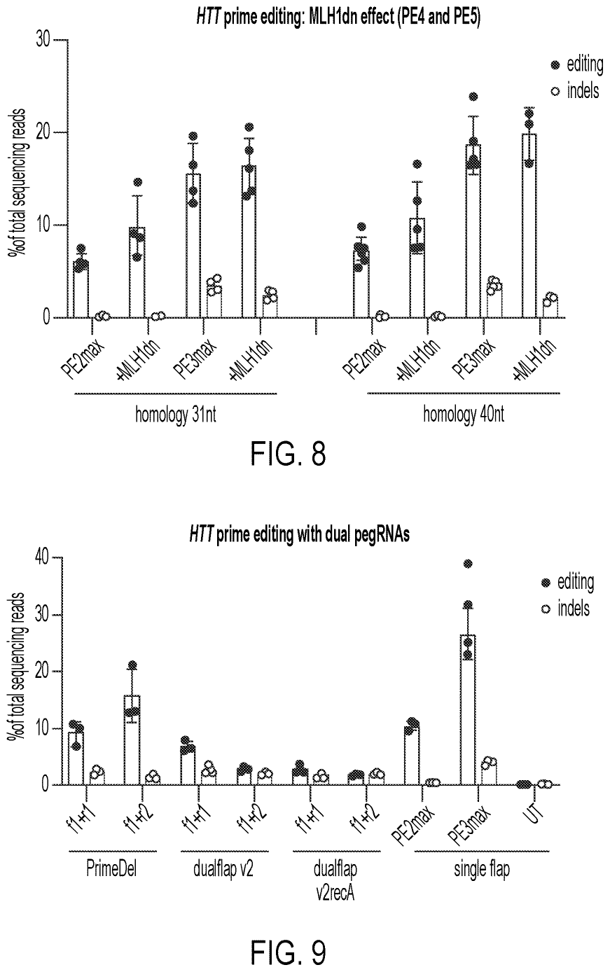

The length of the epegRNA homology sequence was then revisited, and it was determined that epegRNAs with homology of 31 and 40 nt are most efficient (FIG. 6). It was also validated that “PEmax” optimized editor architecture resulted in better prime editing at this locus (FIG. 7). Significant improvement of editing while using these editors in a combination with overexpression of dominant negative MLH1 protein (PE4 and PE5, without and with an additional nicking sgRNA respectively) was not observed (FIG. 8)²⁷. All conditions discussed moving forward were performed with the PEmax editor.

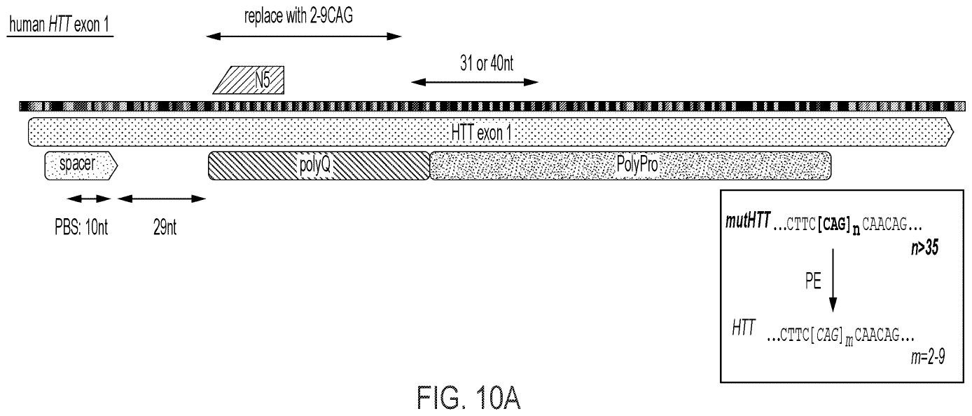

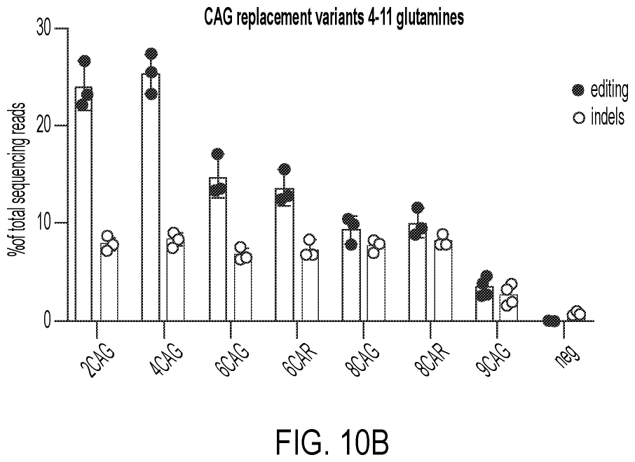

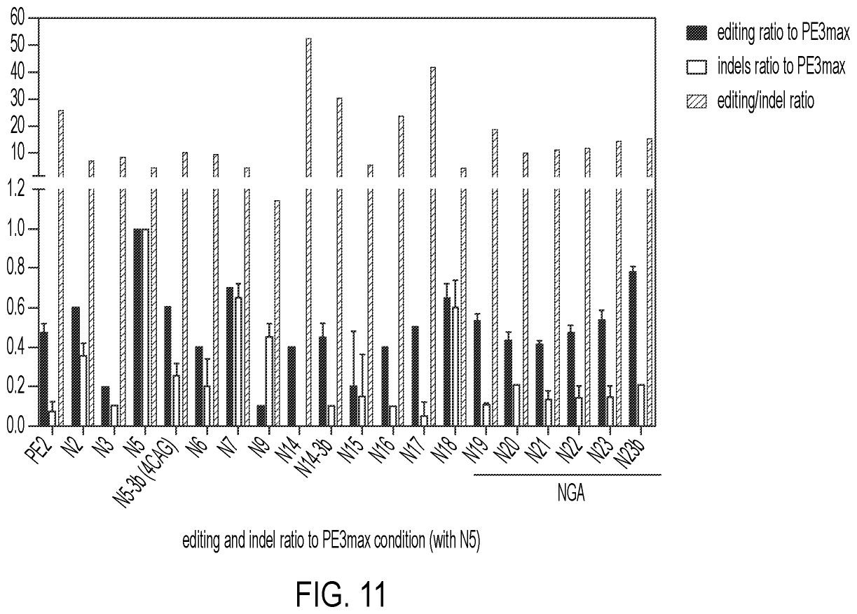

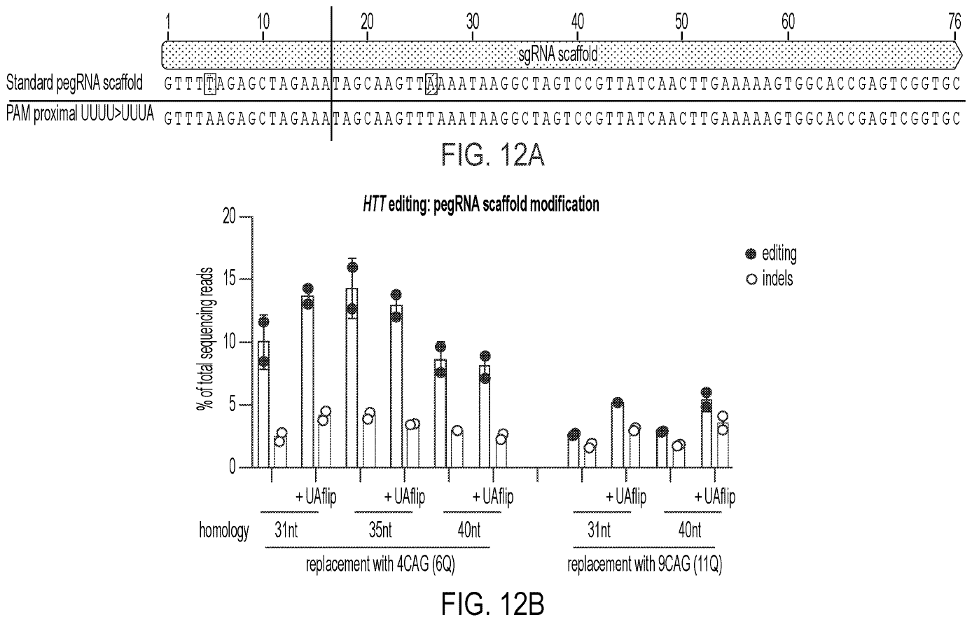

To explore additional pegRNA design options, multiple dual epegRNA strategies were screened, including PrimeDel²⁸ and TwinPE²⁹, and no improvement was observed in comparison to the single epegRNA strategy (FIG. 10). Next, the optimized epegRNA architecture was used in a PE3 system (N5 nick), and different variants of the sequence were screened, replacing CAG repeats (from 2 CAGs to 9 CAGs, either as pure CAG codons or in a combination with interrupting CAA codons—“CARs”, where R stands for “either A or G”). It was observed that replacement of the repeats with 4CAG units results in the highest editing efficiency (FIGS. 10A-10B). To further maximize editing efficiency and maximize the editing: indel ratio in the PE3 system, a few more nicking guides were screened, either recognizing the canonical NGG or non-canonical NGA PAMs. It was determined that N5 nicking guide offers the highest editing efficiency, but N23b nicking guide can be a safer alternative (resulting in lower indels, but also slightly decreased correct editing) if using a PE3b editing system is desired (FIG. 11)¹². Next, the efficiency of epegRNAs with a regular scaffold was compared to ones with a modified scaffold containing a UA flip. This improvement was implemented for all future applications (FIGS. 12A-12B)³⁰.

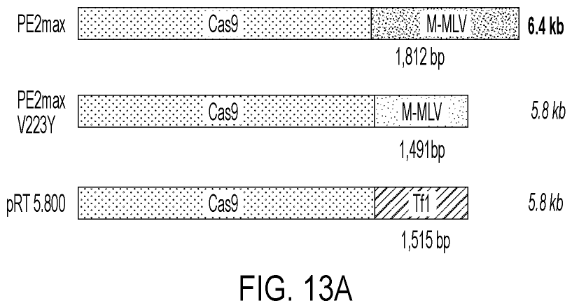

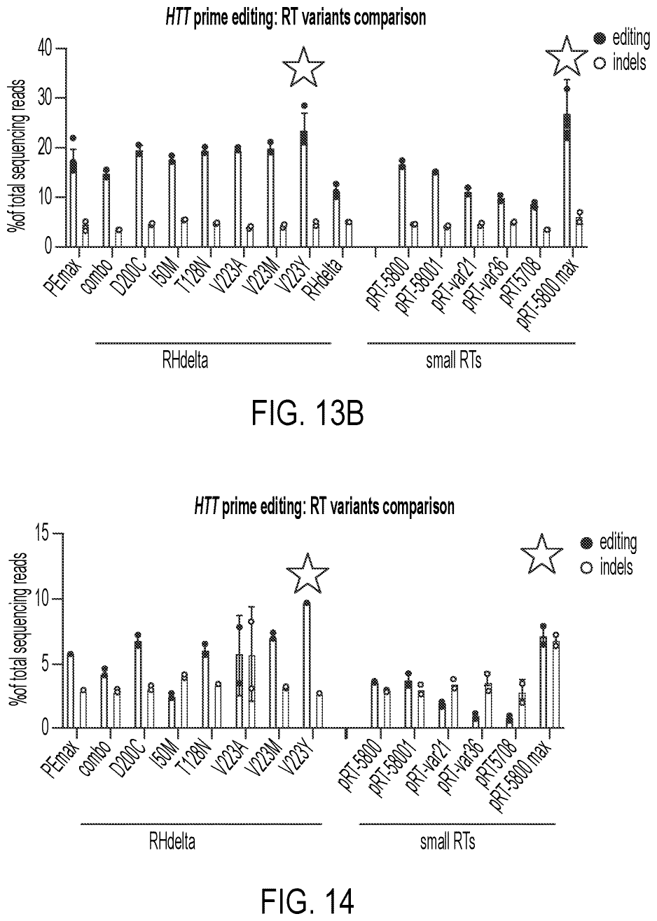

A screen of various different prime editors that employ evolved or rationally designed reverse transcriptase (RT) components was performed next. It was determined that a PE variant with a truncation of the RNAseH domain (e.g., between D497 and I498 of SEQ ID NO: 33) and V223Y mutation in its RT (“V223Y”), as well as pRT-5.800 editor with Tf1 RT (“pRT-5.800max”), in a combination with the optimized epegRNA and nicking guide (N5) resulted in editing significantly higher than PEmax, most likely due to their higher activity and processivity. Additionally, these two editors are much smaller in size, which is advantageous for in vivo applications. This trend was true for short and long CAG replacement variants (FIGS. 13A-13B and 14).

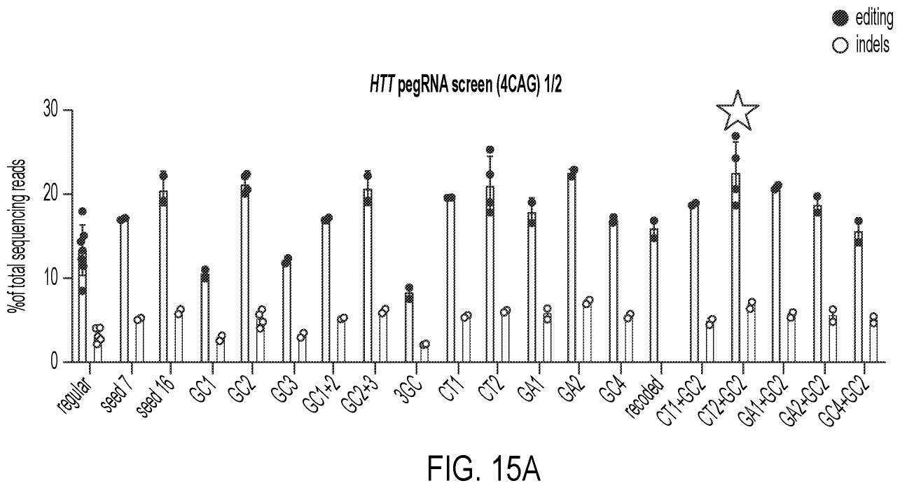

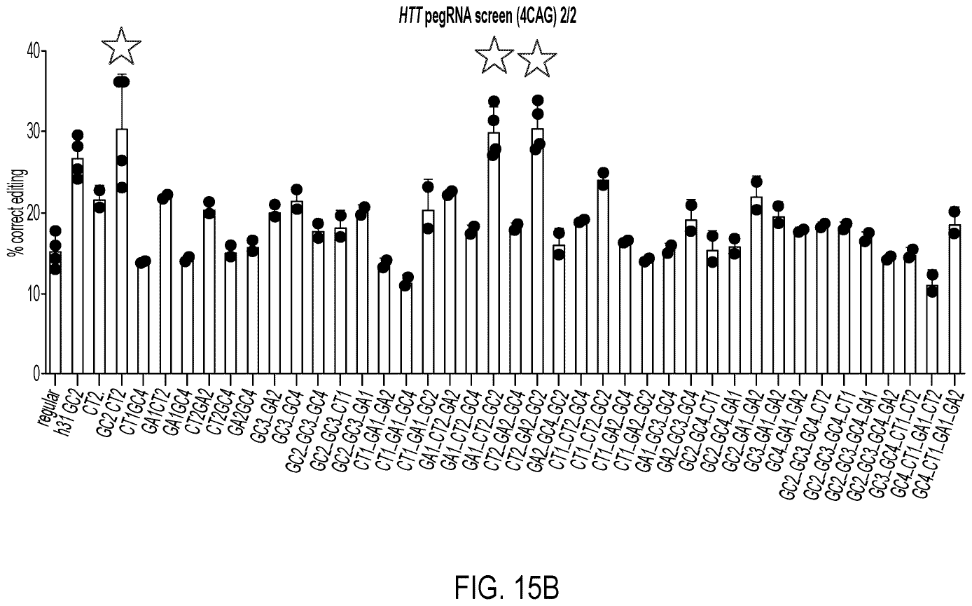

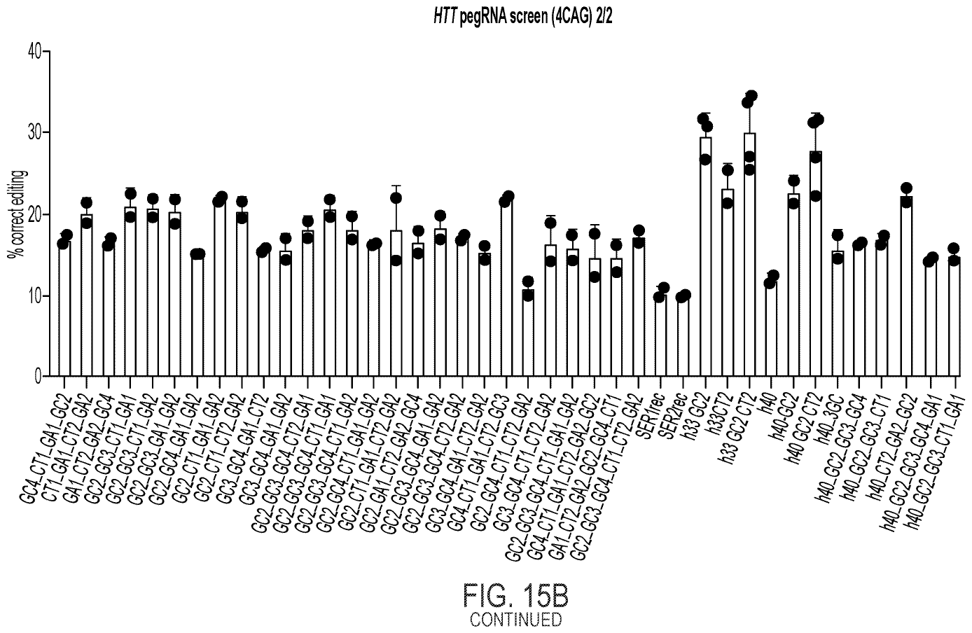

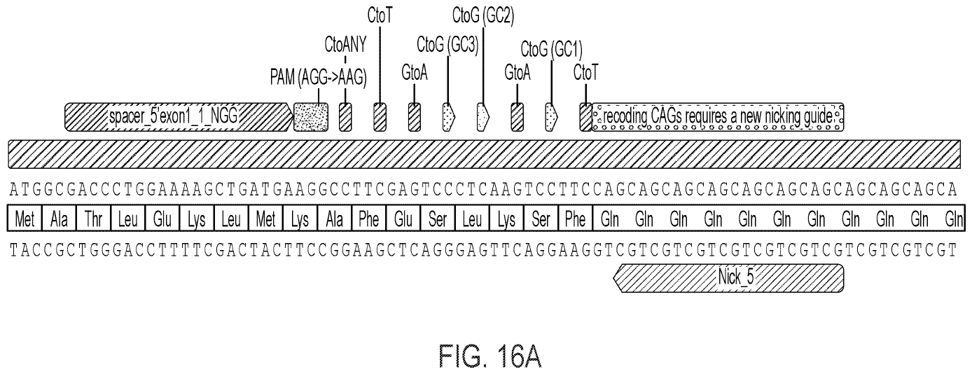

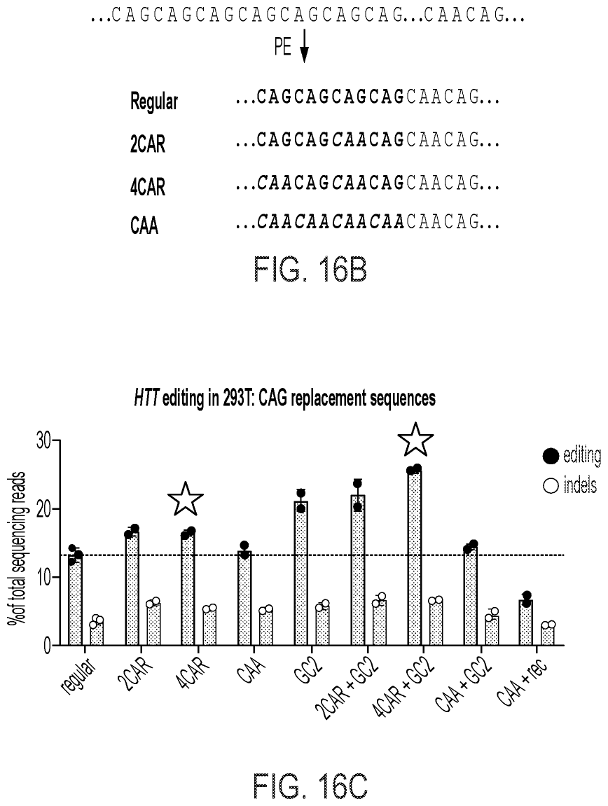

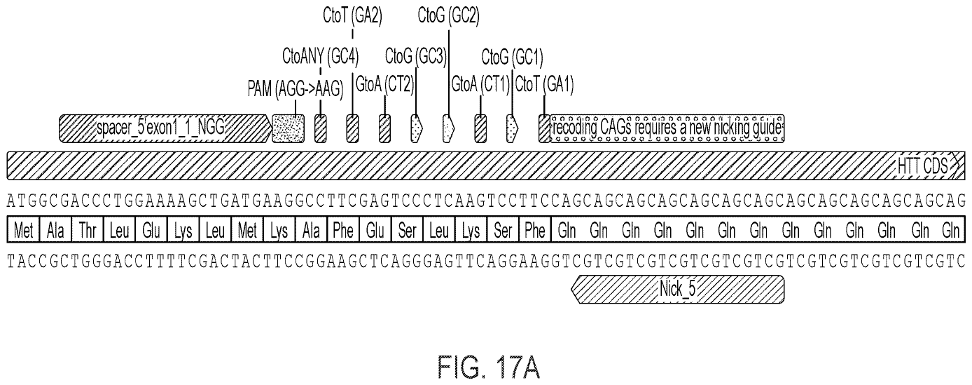

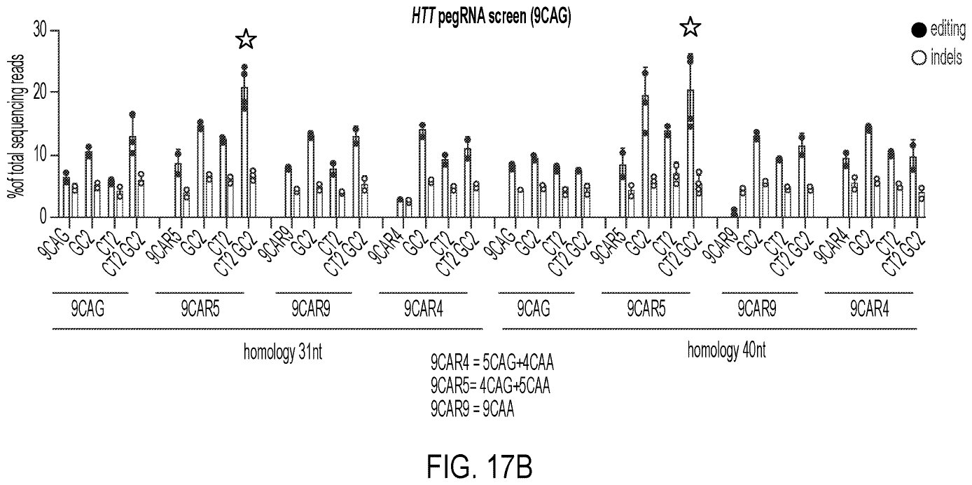

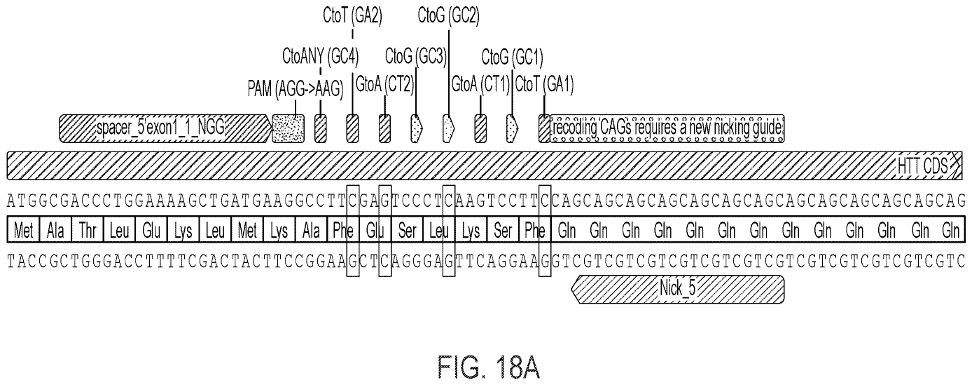

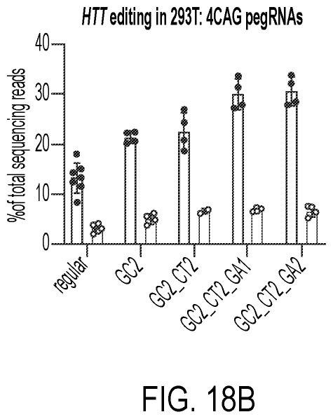

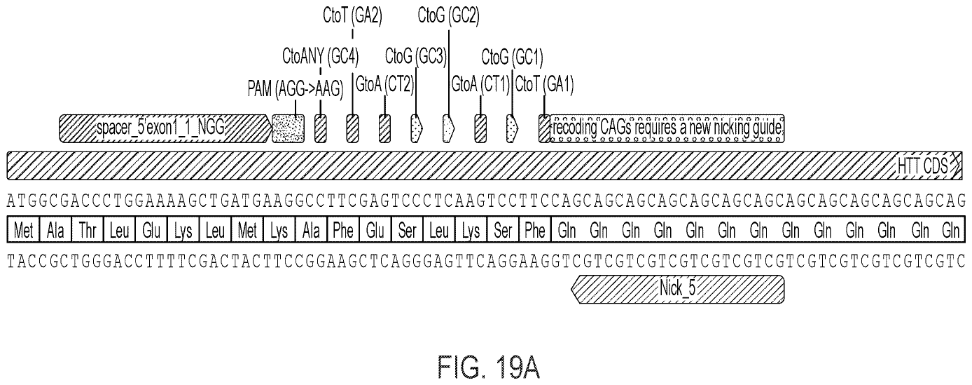

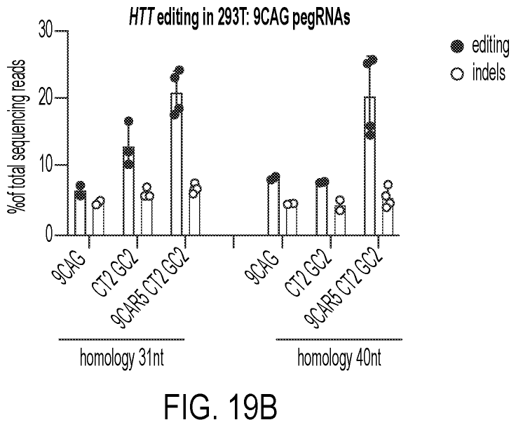

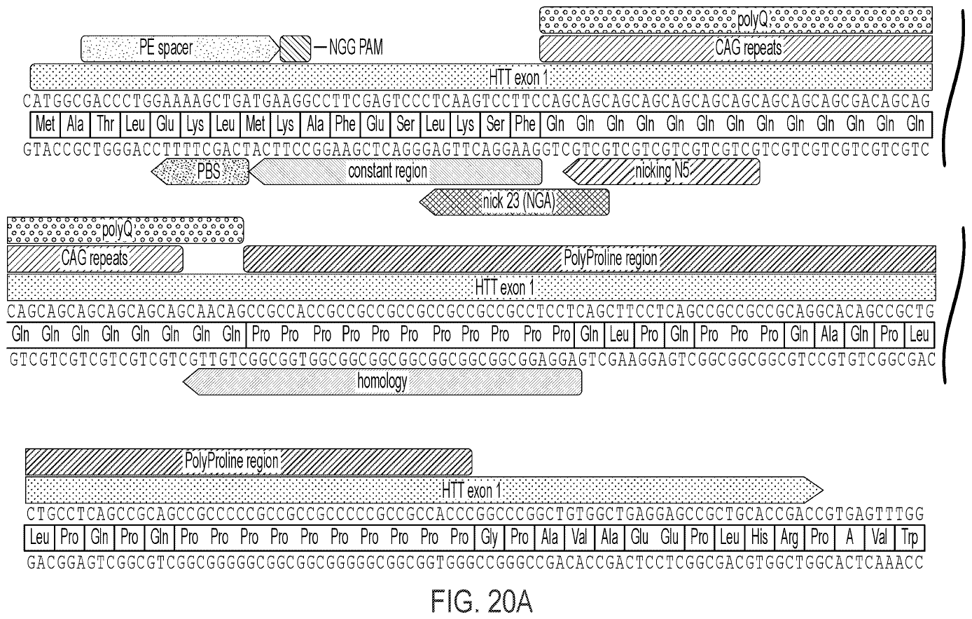

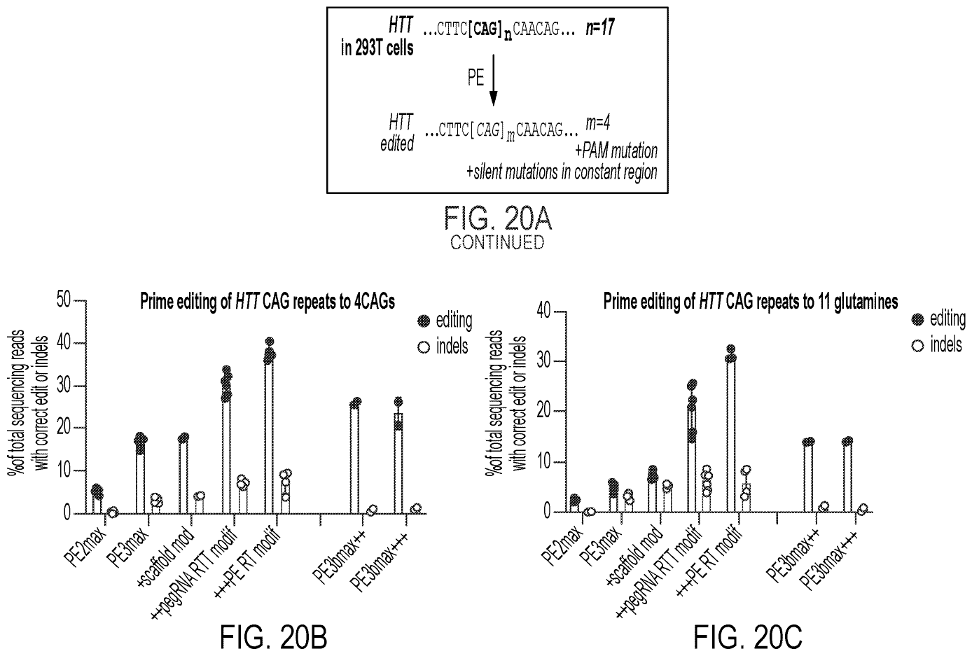

Finally, an extensive epegRNA screen was performed in which silent mutations were introduced in the RTT sequence (i.e., in the synthesis template portion of the RTT) to relax the secondary structure of the RNA and, consequently, allow for a more efficient reverse transcription of the RTT template sequence. A few mutations that resulted in considerably higher editing efficiency were found for both, 4CAG/4CAR (pure CAG repeats or a mixture of CAG and CAA codons, followed by endogenous CAACAG sequence, resulting in six glutamines total) and 9CAG/9CAR (pure CAG repeats or a mixture of CAG and CAA codons, followed by endogenous CAACAG sequence, resulting in eleven glutamines total) replacement strategies (FIGS. 15A-15B, 16A-16C, and 17A-17B). It was found that epegRNA reducing CAG repeat size to 4CAG repeats encoding additional GC2+CT2+GA1 mutations improves editing ~2 fold over the previous epegRNA design (homology 31 nt, PBS 10 nt, epegRNA, UA scaffold flip) in the PE3 system (with N5 nicking guide) (FIGS. 18A-18B). Additionally, epegRNA reducing CAG repeat size to 9CARs encoding additional GC2+CT2 mutations improves editing >2-fold over the previous epegRNA design (homology 31 nt or 40 nt, PBS 10 nt, epegRNA, UA scaffold flip) in the PE3 system (N5 nicking guide) (FIGS. 19A-19B).

In summary, the final prime editing strategy to reduce the size of CAG repeats in the HTT locus employs the following improvements: PE3 or PE3b system¹², PEmax²⁷, epegRNA²⁶, UA scaffold flip³⁰, pegRNA RTT sequence modification, and PE RT variants. All the data presented has been analyzed with software—CAGspresso—developed for this study.

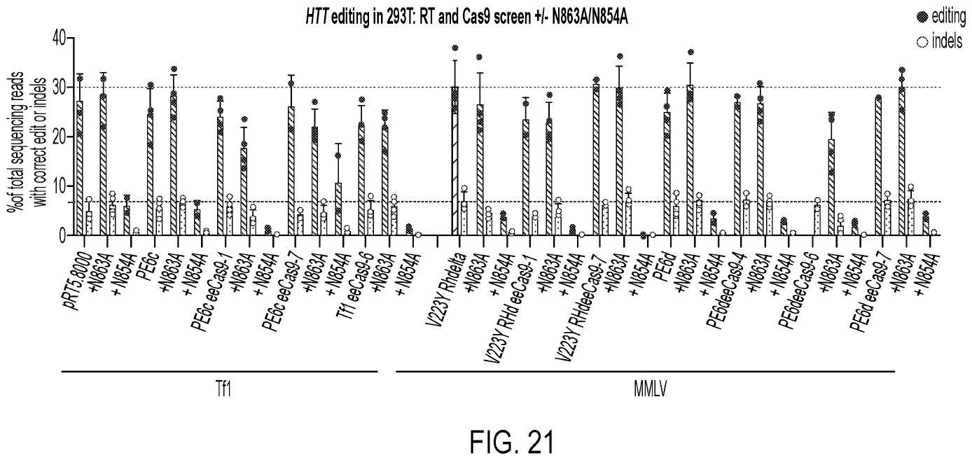

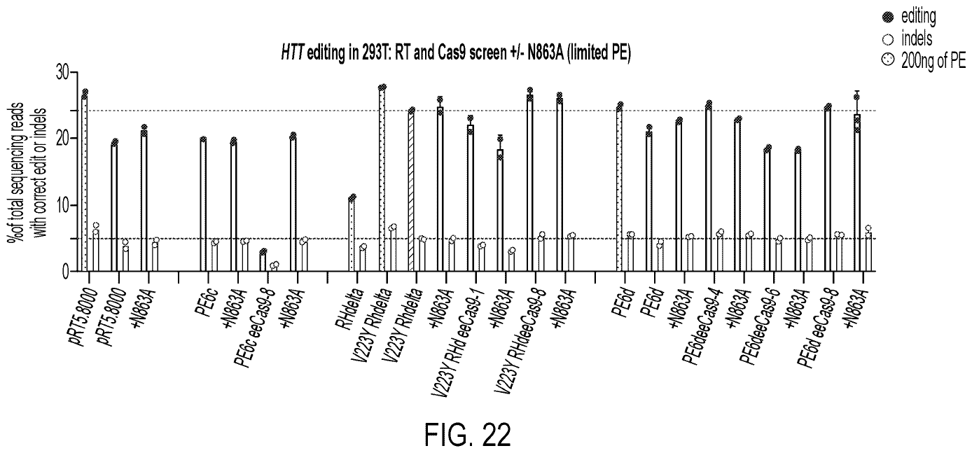

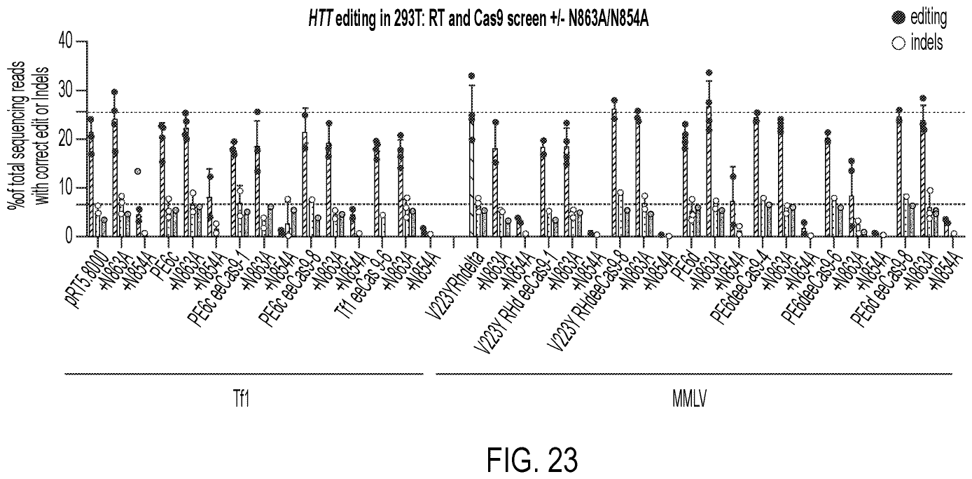

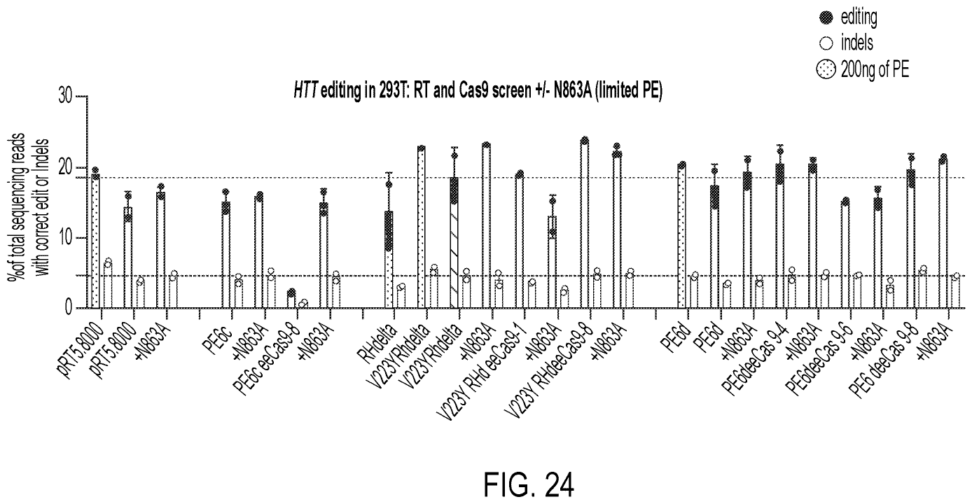

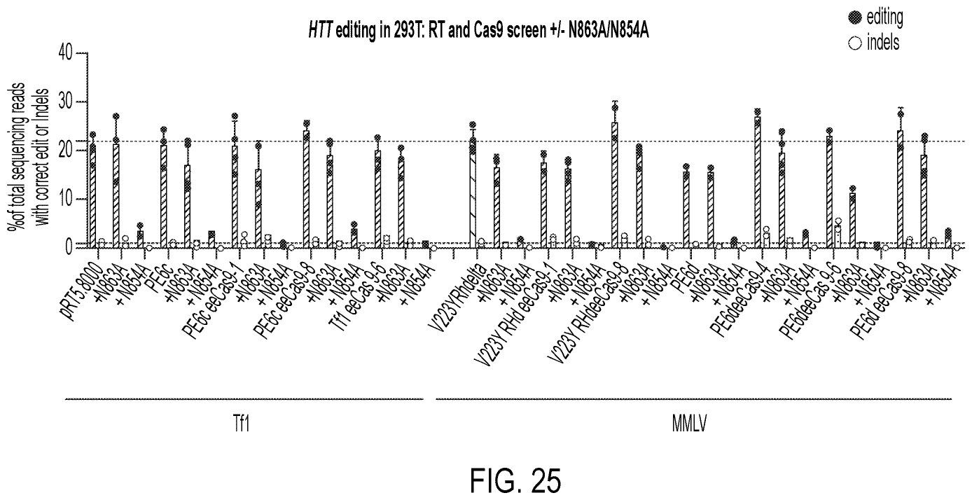

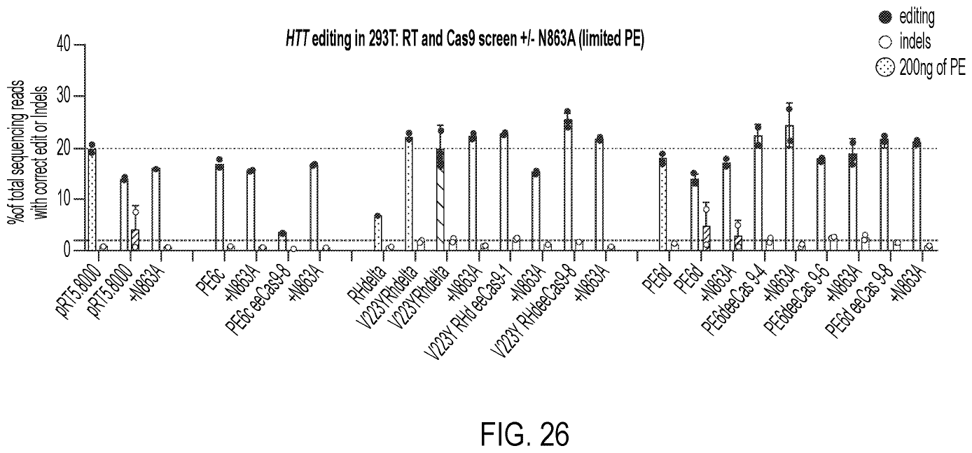

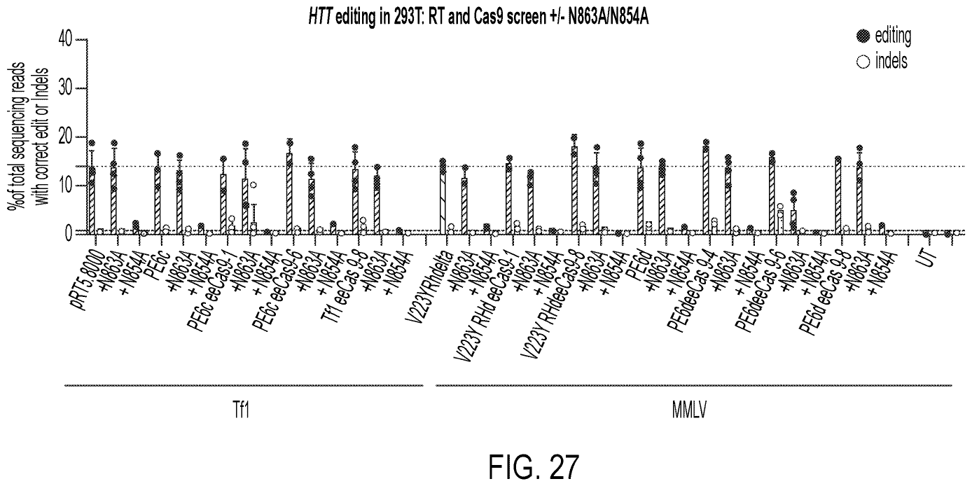

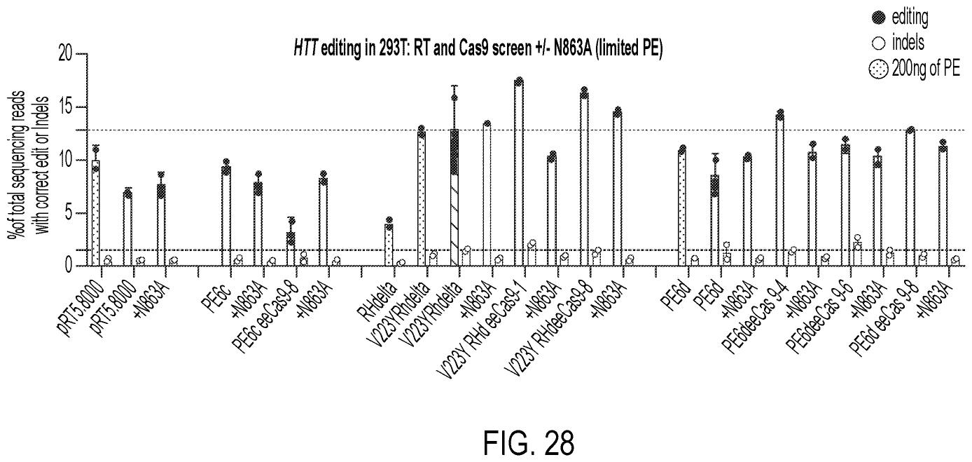

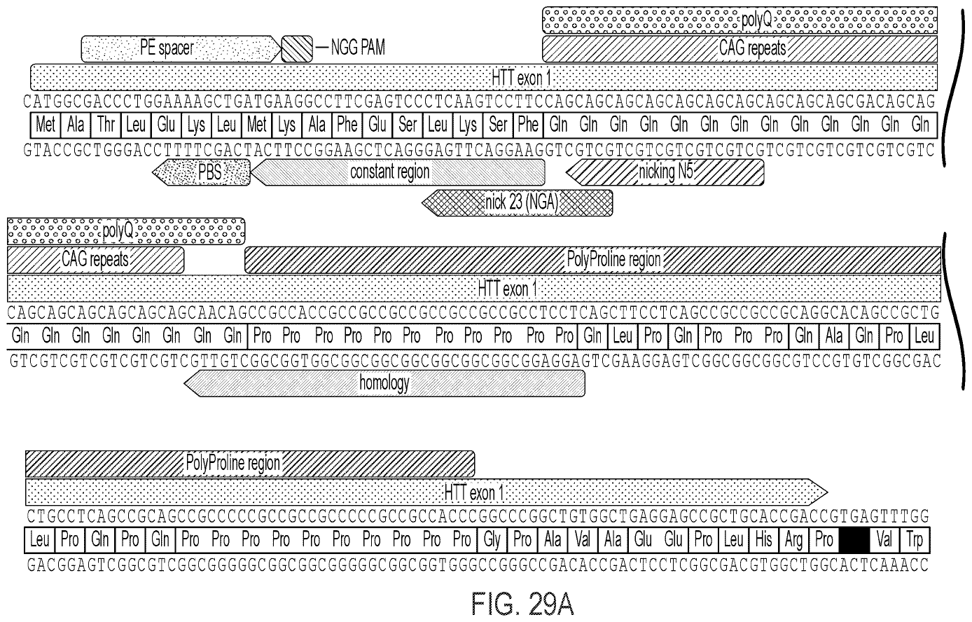

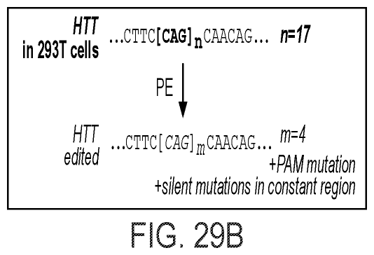

Next, prime editors comprising Cas9 variants were tested in an effort to find a more efficient prime editor variant that would increase editing for both the PE3 and PE3b strategies for 4CAG and 9CAG replacement (FIGS. 21-28). To make the differences in PE performance more visible, PE plasmid input was decreased to a concentration that becomes limiting for prime editing, and the different PE variants were compared under this more stringent condition. Two Cas9 variants were identified: “eeCas9-1” (SpCas9 with K918A+K775R) and “eeCas9-8” (SpCas9 with D23G+H754R) in a combination with truncated MMLV (RnaseH deletion) with a V223Y mutation (V223YRHdelta) showed higher editing efficiency than a prime editor comprising the V223YRHdelta variant by itself. FIGS. 29A-29B provide an updated summary of the PE strategy to reduce CAG repeats in view of the additional Cas9 variants tested.

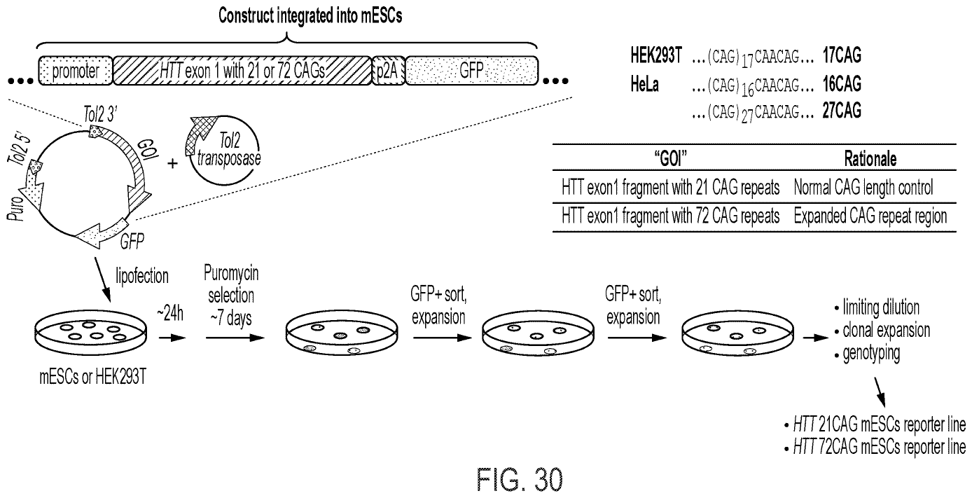

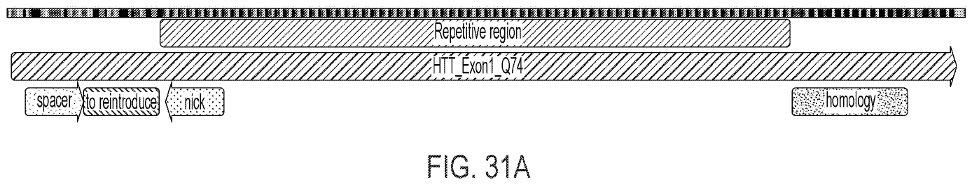

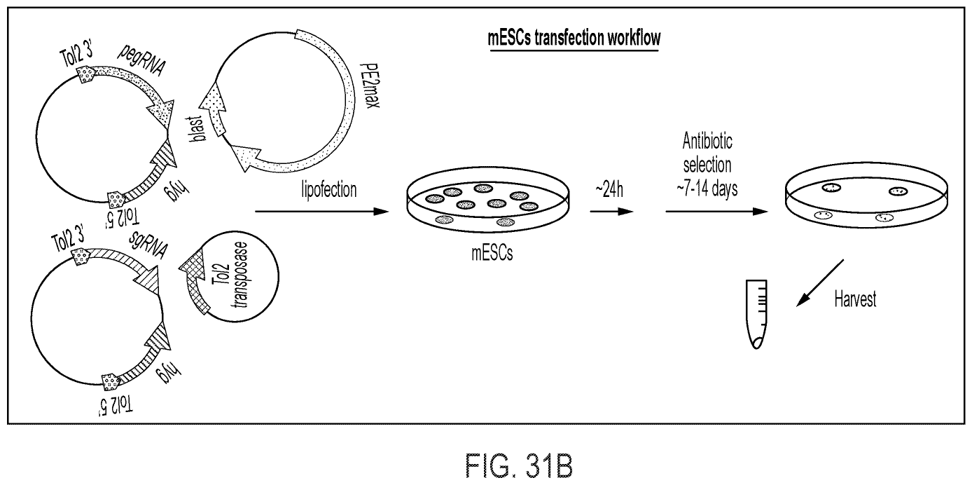

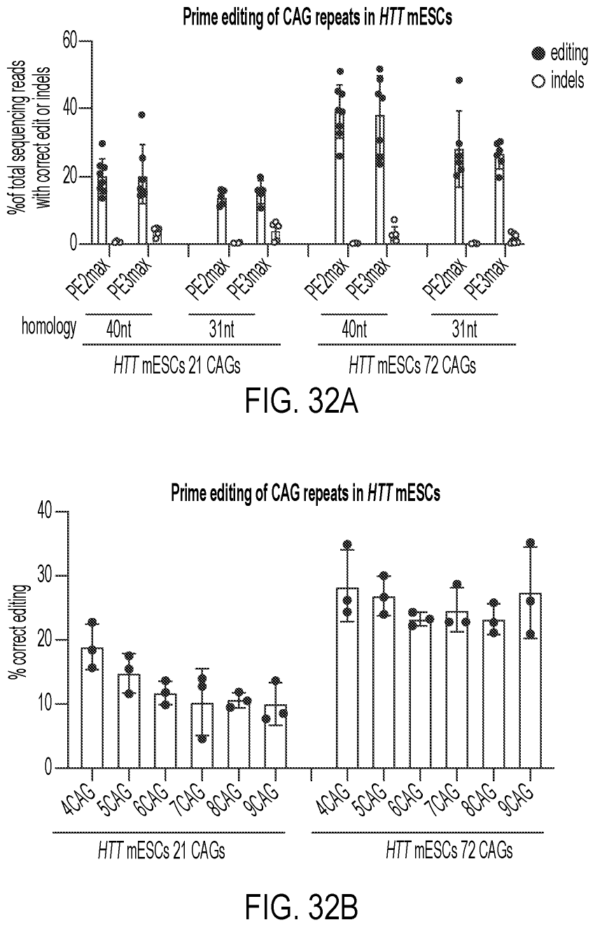

To validate the prime editing strategy in more relevant cell lines, transgenic mouse embryonic stem cell lines (mESCs) containing the human HTT exon 1 were generated. Using Tol2 transposase, two cell lines expressing HTT exon 1 with either 21 or 72 pure CAG repeats³¹, ending in a terminal ‘CAACAG’ sequence^{32,33}, were generated. These transgenic cell lines allowed rapid screening of genome editing at both non-pathogenic (21 CAG) and pathogenic (72 CAG) repeat lengths, that are larger than CAG repeats at endogenous HTT alleles typically found in wild type human cell lines (typically 16-18 CAG) (FIGS. 30 and 31A-31B). Notably, prime editing at shorter CAG repeats in HTT-mESC was lower (22% at 21 CAGs), suggesting prime editing may be more efficient at pathogenic repeat lengths than normal alleles, possibly due to unfavored secondary structure formation of long repeat alleles (FIGS. 32A-32B). Editing efficiency was somewhat decreased when pegRNAs encode the incorporation of a greater number of CAGs (FIG. 32C).

Next, the prime editing strategy to reduce CAG repeats was tested in a HD mouse model with expanded CAG repeats. An Htt. Q111 mouse model was used that carries a human pathogenic HTT allele with ~111 glutamines and exhibits somatic repeat instability in the CNS (including the striatum and cortex) and the liver^{71-73.}

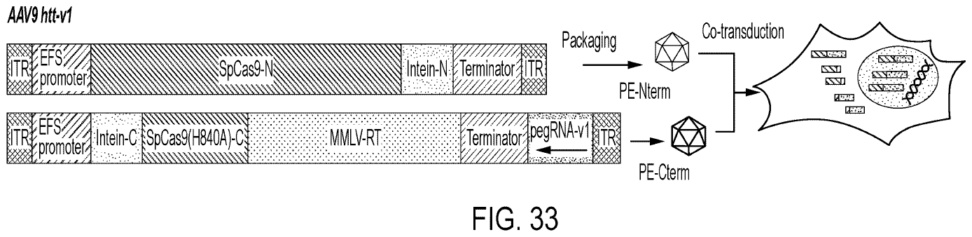

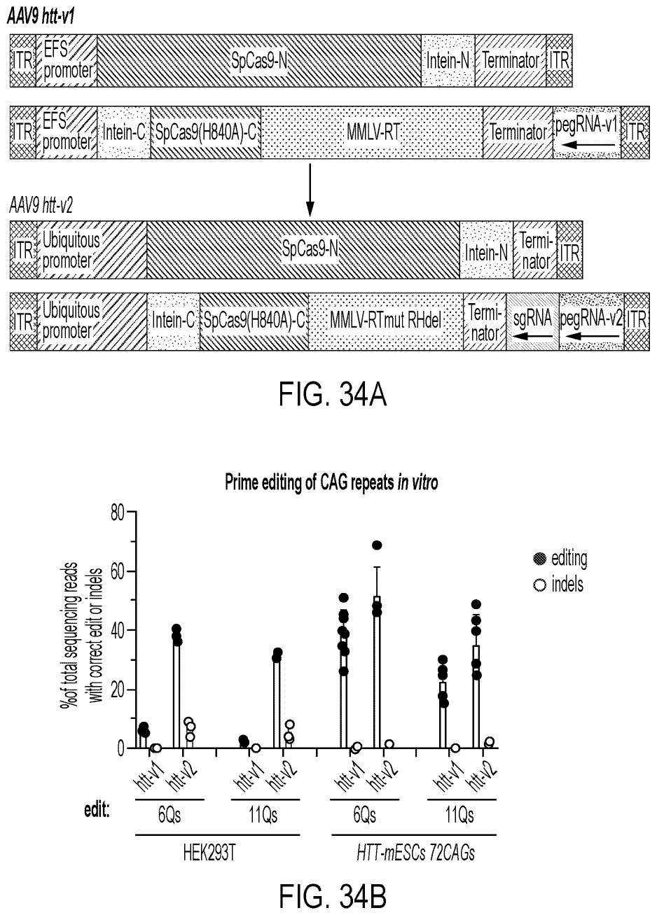

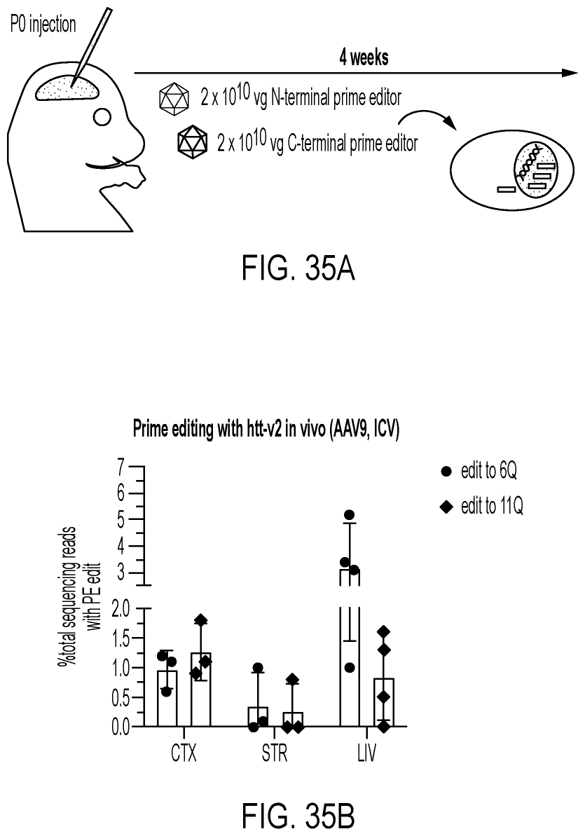

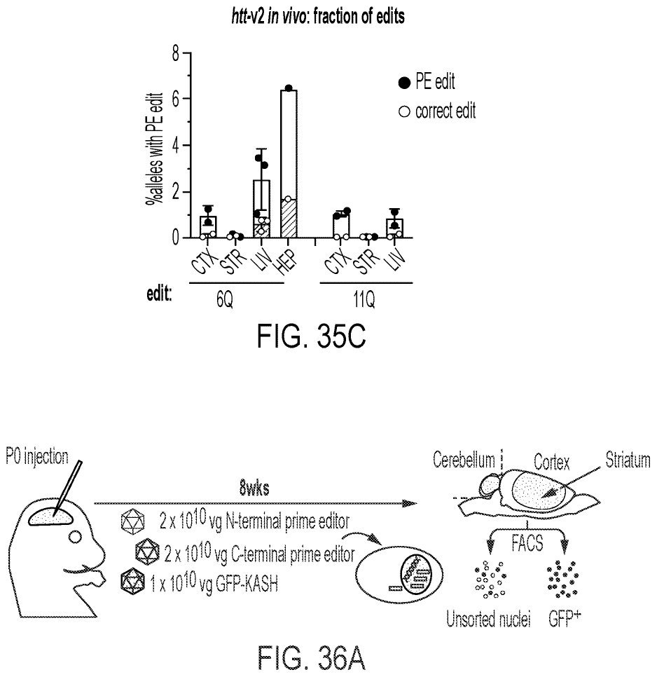

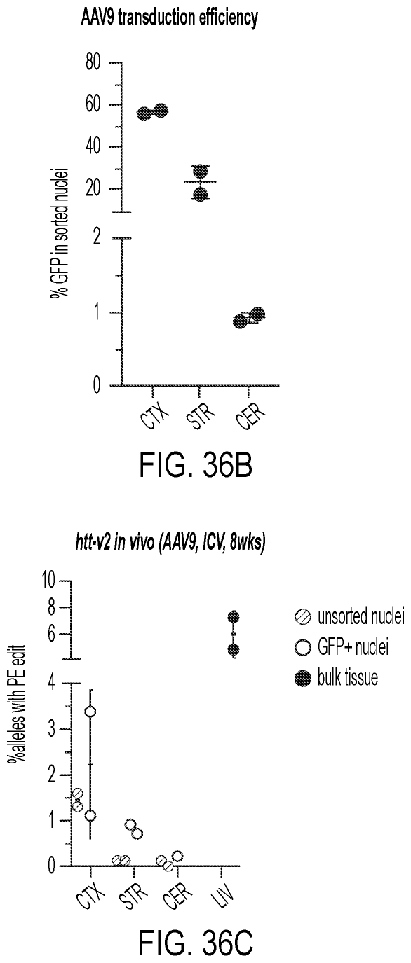

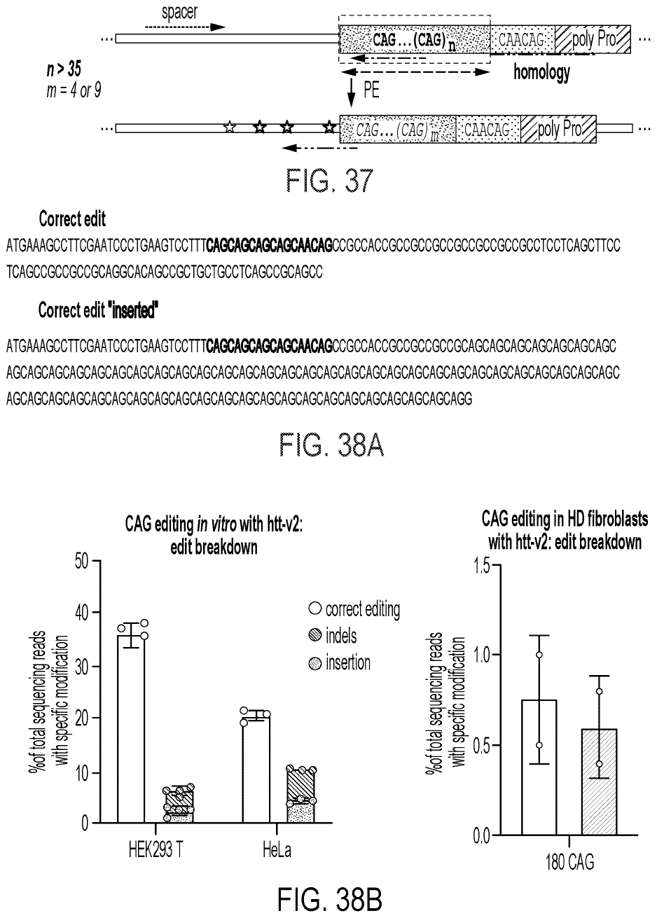

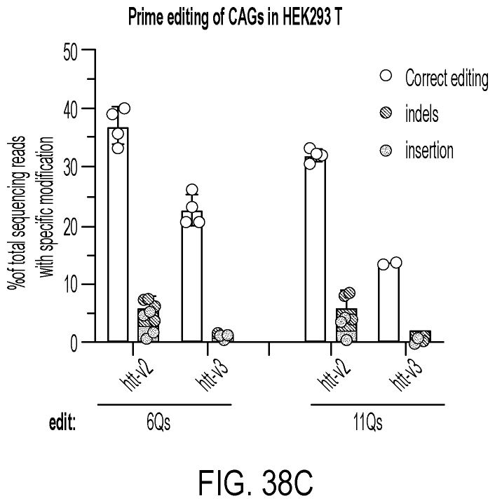

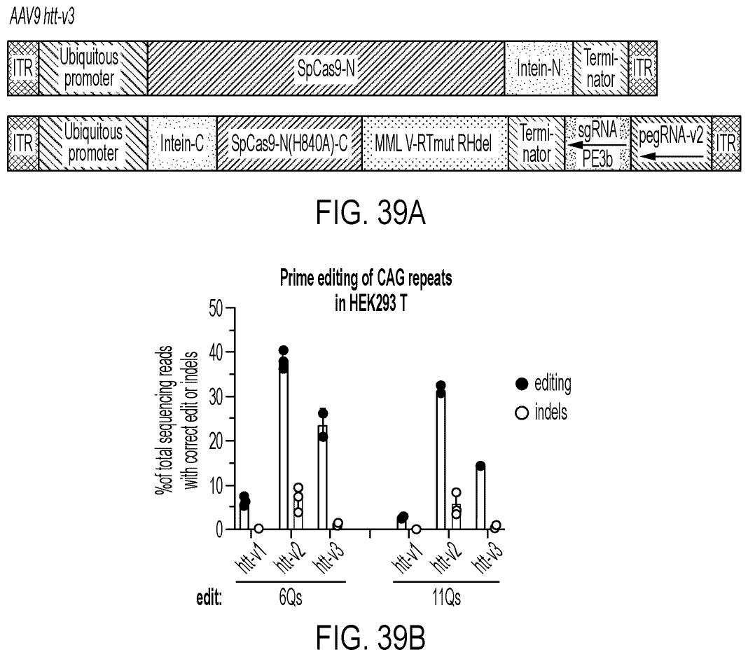

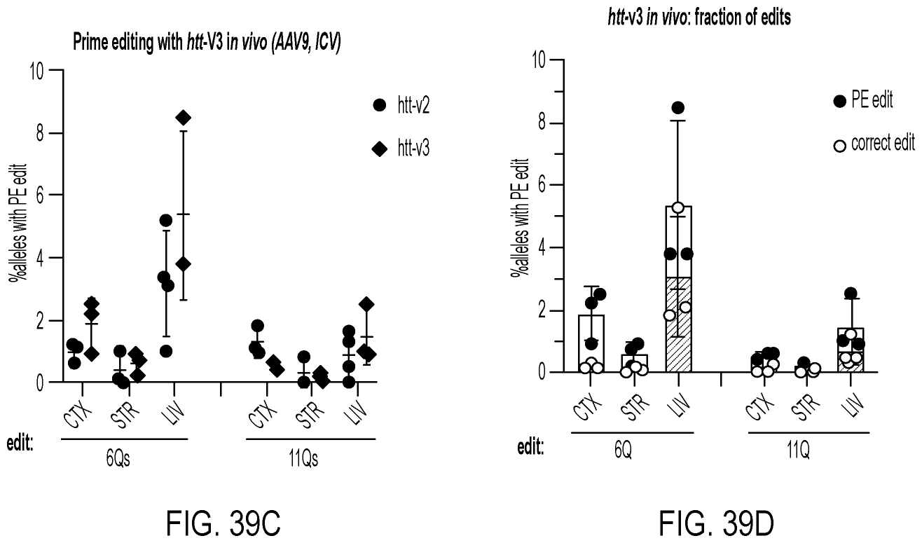

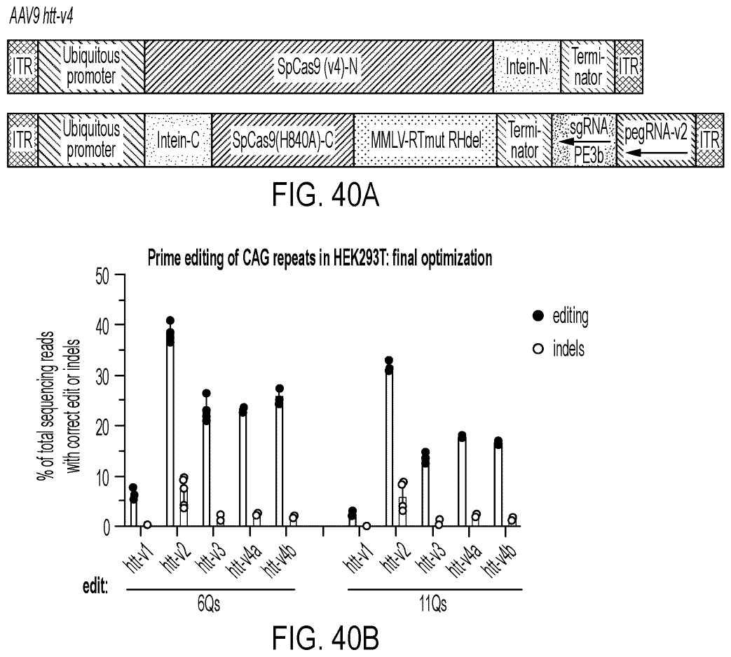

A dual-ssAAV delivery system was generated for the HTT-targeting prime editing strategy to replace the pathogenic number of CAG repeats in Htt.Q111 with either 6 or 11 glutamines^{54,74,75}. In the first round of preliminary optimizations, intracerebroventricular (ICV) injections were performed in Htt.Q111 neonates using ssAAV9 virus and the htt-v1 (dual ssAAV9-PE-v1). Four weeks post injection, no appreciable prime editing was observed above background (FIG. 33). The AAV architecture was reevaluated, and a new prime editing strategy (htt-v2) that utilizes additional improvements to the prime editor and pegRNA was used (FIGS. 34A-34B). P0 ICV was injected into HTT.Q111 neonates with a regular dose of dual ssAAV9-PE-v2 (4.0×10¹⁰ vg total)⁵⁴, and editing was evaluated four weeks post injection. Approximately 1% of alleles in the cortex and over 3% of alleles in the liver acquired the desired prime edit (FIGS. 35A-35C). However, it was determined that the majority of the edited alleles still carry the endogenously encoded pathogenic CAG repeats (FIGS. 35A-35C). The treatment with ssAAV9-PE-v2 was extended, and transduction efficiency in treated animals was assessed. Eight weeks after the injection, ~60% transduction efficiency in the cortex and ~20% GFP+ flow-sorted nuclei in the striatum was observed (FIGS. 36A-36C). The editing observed in the examined tissues increased ~2-fold compared to the 4-week time point (FIGS. 36A-36C and 37). As the transduction efficiency did not seem to be limiting, the alternative PE3b prime editing strategy was next evaluated (htt-v3, described above), which yielded good editing efficiency with much lower indels (FIGS. 38A-38C). By using this new approach (dual ssAAV9-PE-v3), the goal was to reduce undesired editing outcomes and possibly increase editing efficiency in vivo (FIGS. 38A-38C). The mice were P0 ICV injected with a high dose (1.6×10¹¹ vg total) of the dual ssAAV9-PE-v3 to compensate for the lower editing efficiency of the PE3b system compared to PE3 observed in vitro, and the editing outcomes were assessed 4 weeks post injection. Compared to htt-v2, strategy htt-v3 showed a slight increase in % alleles with the prime edit, including up to 2-fold in the cortex and liver (FIGS. 39A-39D). Most edited alleles in the cortex and striatum still contained the endogenous CAG repeats (FIGS. 39A-39D). The fraction of correctly edited alleles improved, however, for the liver, in which >50% of all prime edits did not contain the pathogenic CAG tract (FIGS. 39A-39D). This unexpected editing outcome may be caused by 1) DNA damage repair mechanisms resolving the transient DSB at the edited site before editing is complete; 2) unsuccessful flap resolution after the desired sequence (edit) gets incorporated into the DNA; or 3) inefficient prime editing in the transduced cells caused by a non-optimal strategy design and/or the complex character of the edit. To address these potential limitations, additional evaluation of the prime editing strategies in vitro was performed by testing new PE variants to further improve editing. It was found that some strategies (htt-v4a and htt-v4b), have the potential to perform better in the endogenous HTT context. These strategies were shown to yield slightly increased editing efficiency in vitro (FIGS. 40A-40B).

### Example 2. Using Prime Editing to Remove Long GAA Expansions at Pathogenic FXN Alleles

The length of FXN GAA-repeats in the general population ranges from ~5-60, while FRDA patients may present with 66 to well over 1200 repeats, typically ranging from 600 to 900 repeats⁵⁷. The age of FRDA onset in patients, loss of FXN protein, and severity of symptoms are inversely correlated with the GAA repeat length of the shortest FXN allele (FIG. 41).

The first intron in FXN plays a key role in transcriptional regulation, and GAA repeat expansion induces epigenetic changes to the chromatin state that results in silencing of gene expression⁸¹⁻⁸³. Excision of the locus containing the expanded GAA repeats has been demonstrated to increase the total mature FXN transcripts in cells and thereby enable rescue of FXN protein levels and some disease phenotypes in FRDA patient cell lines^{64-66, 84, 85}, however, due to technical considerations regarding target sequence specificity, strategies result in large (1-20 kb) deletion of the surrounding sequence, including substantial removal of native FXN regulatory sequences, which may impact long-term and tissue-specific rescue of FRDA-afflicted tissues^{86,87}

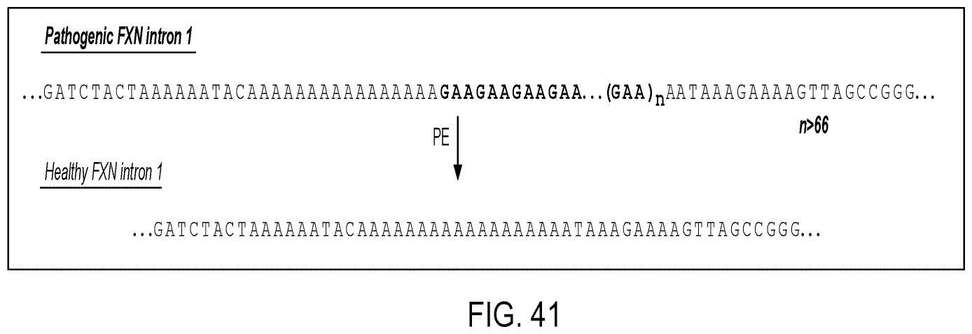

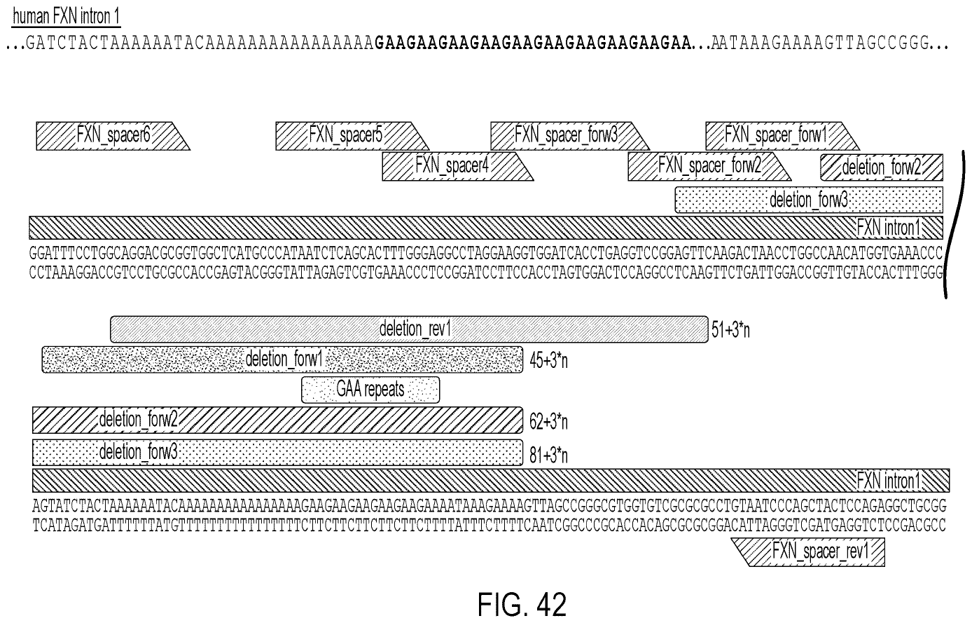

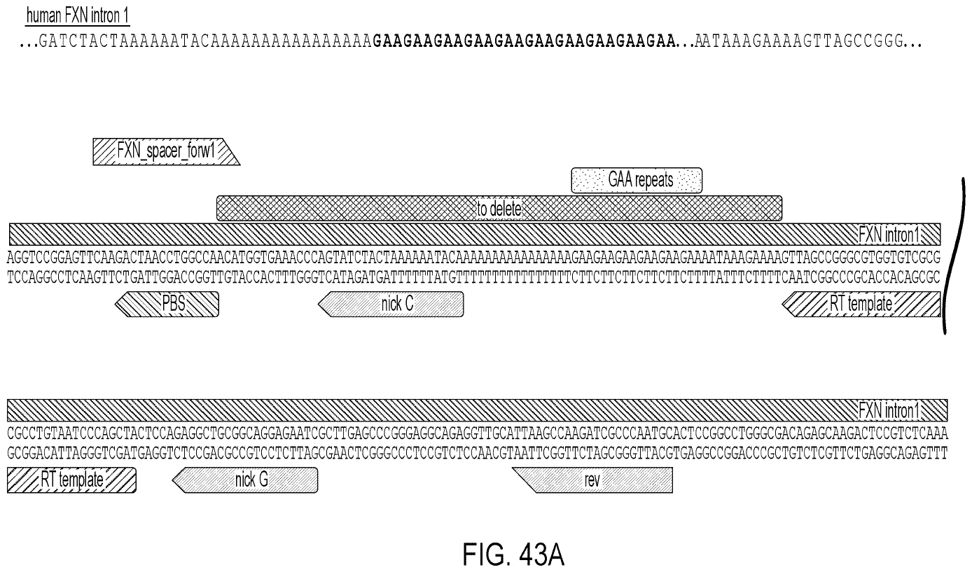

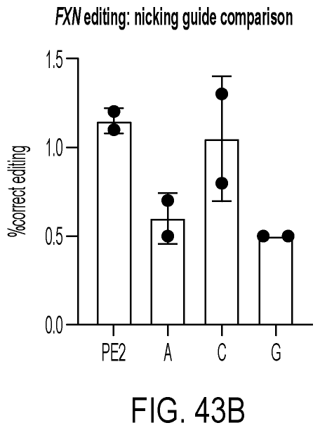

Prime editing⁷³ was explored as a means to precisely modify FXN intron 1 to remove expanded GAA repeats while minimally disrupting surrounding regulatory sequences. A few hundred prime editing strategies (PE2) using various prime editing sgRNA (pegRNA) designs were screened. Multiple spacer sequences paired with different PBS and RT template (RTT: homology+synthesis template sequence) lengths were tested, which allowed selection of the best spacer sequence (here: “spacer”) enabling up to 18% average deletion of GAA repeats at endogenous loci in HEK293T cells (FIG. 42). To increase editing efficiency, PE3 strategies, which pair PE2 editing with an additional sgRNA to induce a nick near or within the editing target site to bias DNA repair towards incorporation of the prime edit were explored^{73,74,88}. Out of three different nicking conditions, one was selected—“nick C,” which resulted in the highest editing efficiency (FIGS. 43A-43B).

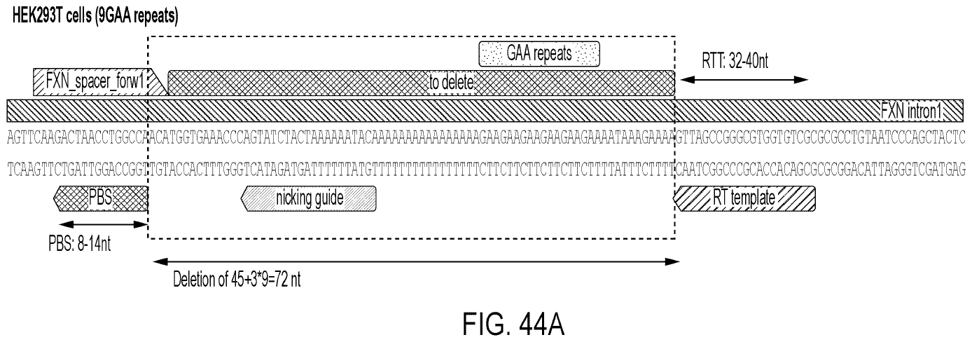

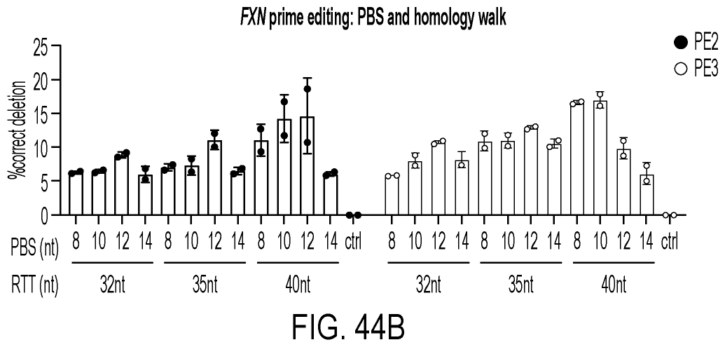

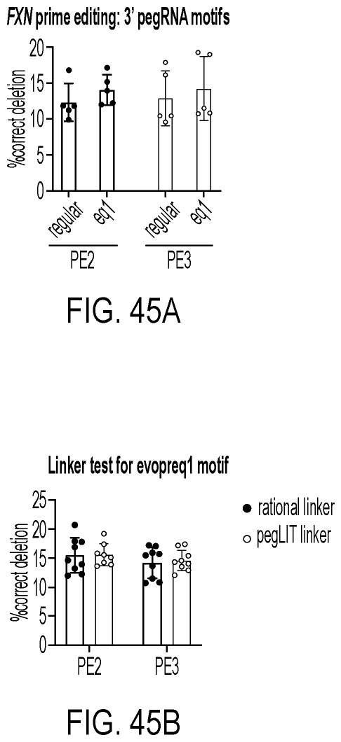

Next, another extensive screen of pegRNA designs with varying PBS and RTT lengths was performed in PE2 and PE3 systems with extra nick C. From all tested conditions, pegRNAs with PBS of 10 nt and homology of 40 nt resulted in the highest prime editing (FIG. 44A-44B). Additional prime editing improvements at this locus were further evaluated. PegRNA with 3′ structural motifs (evopreq1 and mpknot)⁸⁹ were tested with rationally designed linker sequences or linker sequences computed by pegLIT⁸⁹, and a slight increase in editing efficiency was observed after adding an evopreq1 motif with a pegLIT-predicted linker sequence (FIGS. 45A-45B). All pegRNA designs from here forward include the evopreq1 motif and are referred to as “epegRNA.”

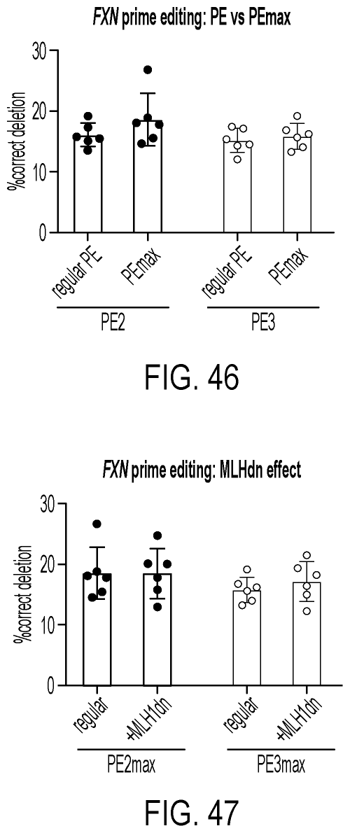

It was then validated that use of PEmax-optimized editor architecture results in slightly better prime editing at this locus (FIG. 46), but no significant improvement of editing was observed while using it in combination with overexpression of dominant negative MLH1 protein (PE4 and PE5, without and with an additional nicking sgRNA, respectively) (FIG. 47)⁹⁰. From here forward, all conditions discussed were performed with PEmax editors.

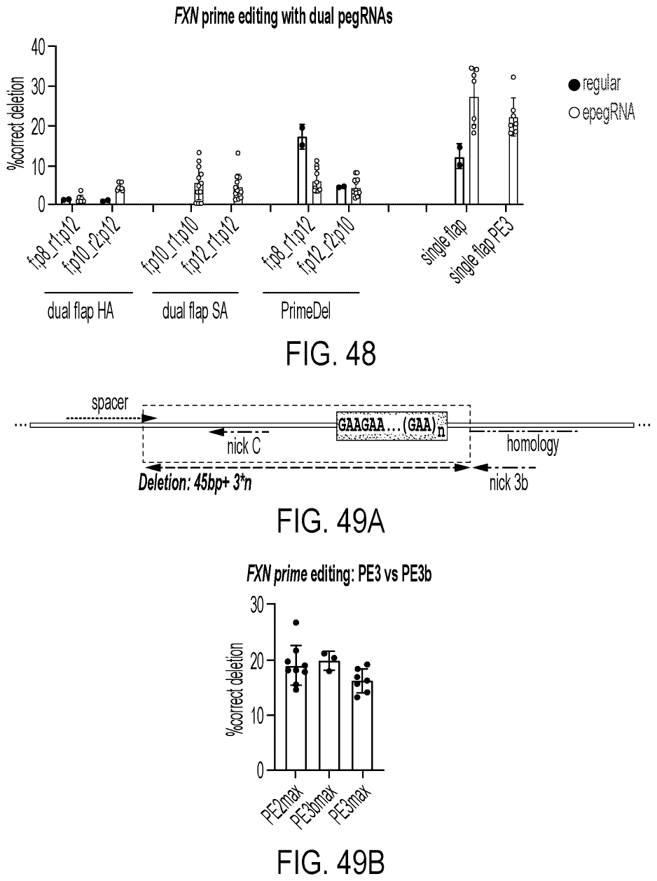

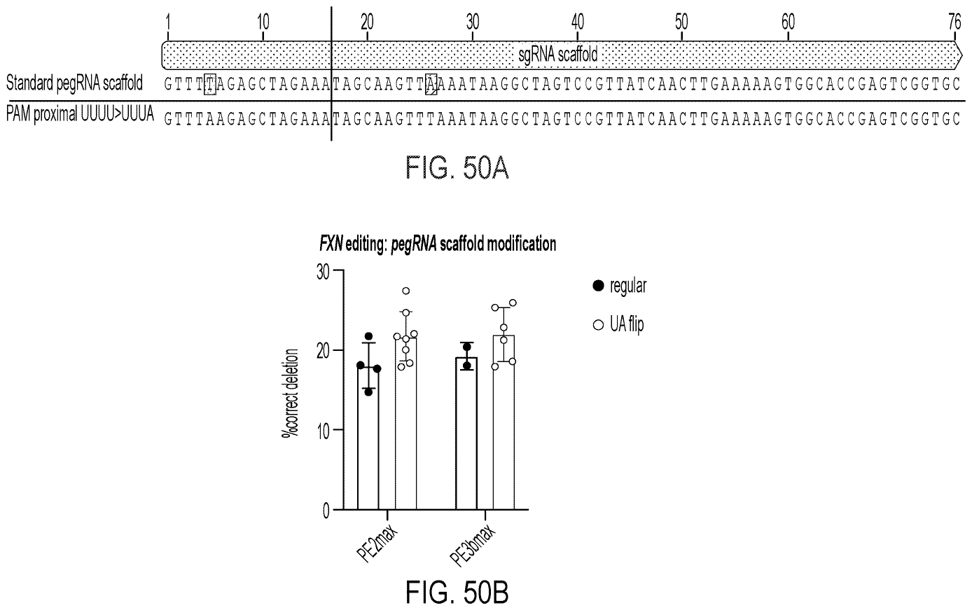

To explore additional pegRNA design options, multiple dual epegRNA strategies were screened, including PrimeDel⁹¹ and TwinPE⁹², and no improvement was observed in comparison to the single epegRNA strategy (FIG. 48). To further maximize editing efficiency and maximize the editing: indel ratio in the PE3 system, additional nicking guides were screened, and it was determined that nicking guide 3b outperforms nick C and offers a safer alternative to the PE3 system, resulting in lower indels (FIGS. 49A-49B)^{73,93}. Next, the efficiency of epegRNAs with a regular scaffold was compared to ones with a modified scaffold containing UA flip, and this improvement was implemented in all pegRNAs tested from here forward (FIGS. 50A-50B)⁹⁴.

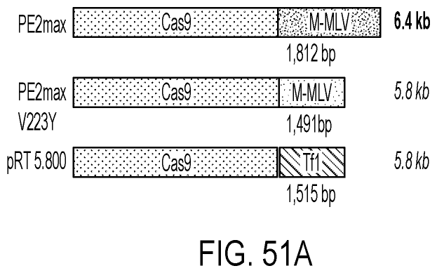

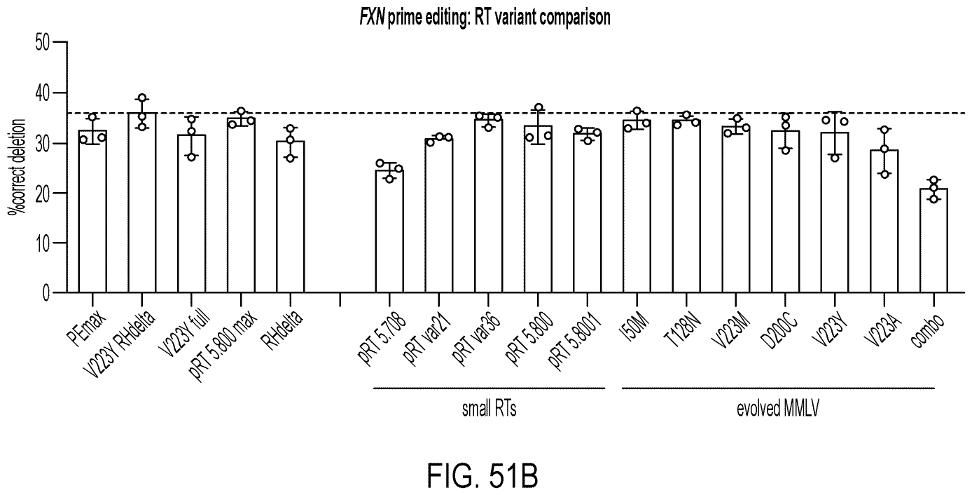

Finally, a screen of various different prime editors that employ evolved or rationally designed reverse transcriptase (RT) components was performed. It was determined that a PE variant with a truncation of the RNAseH domain and a V223Y mutation in its RT (“V223Y RHdelta”), as well as pRT-5.800 editor with Tf1 RT (“pRT-5.800max”) in a combination with the optimized epegRNA and nicking guide (3b) resulted in editing significantly higher than regular PEmax, most likely due to their higher activity and processivity. Additionally, these two editors are much smaller in size, which is advantageous for in vivo applications (FIGS. 51A-51B).

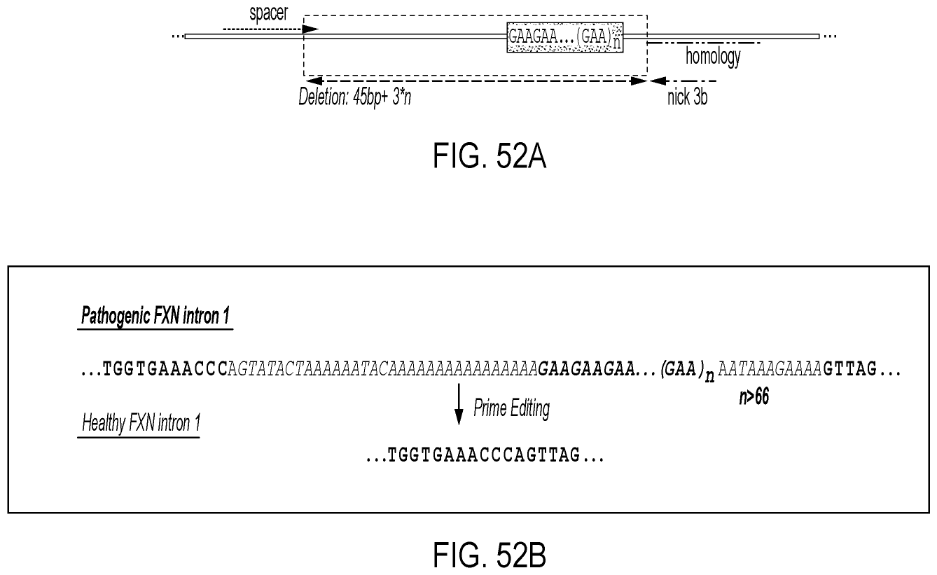

In summary, an optimized prime editing strategy to remove the long GAA repeat expansions in the FXN locus was developed, employing the following improvements: PE3b system^{73,93}, PEmax⁹⁰, epegRNA⁸⁹, UA scaffold flip⁹⁴, and PE RT variants. All the data presented has been analyzed with software—FRATAXino—developed for this study (FIGS. 52A-52B).

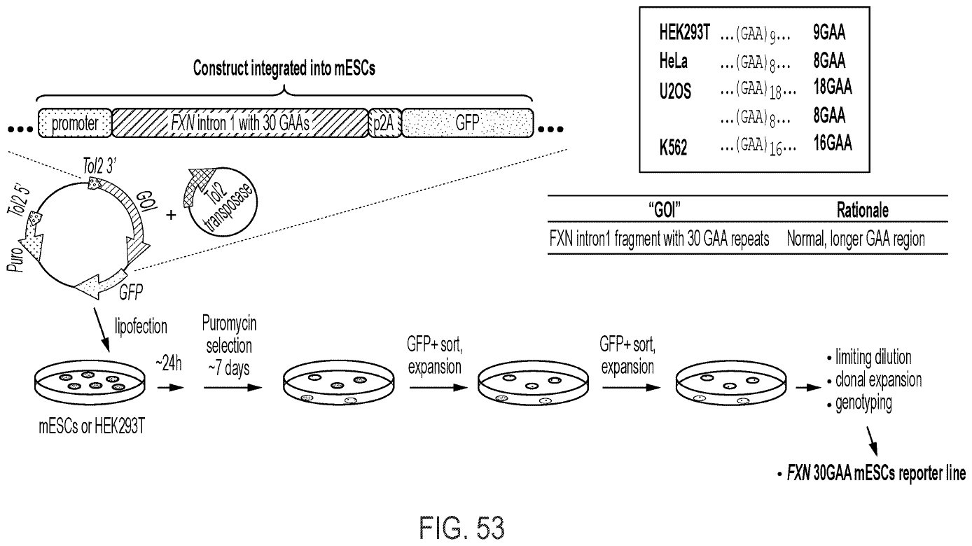

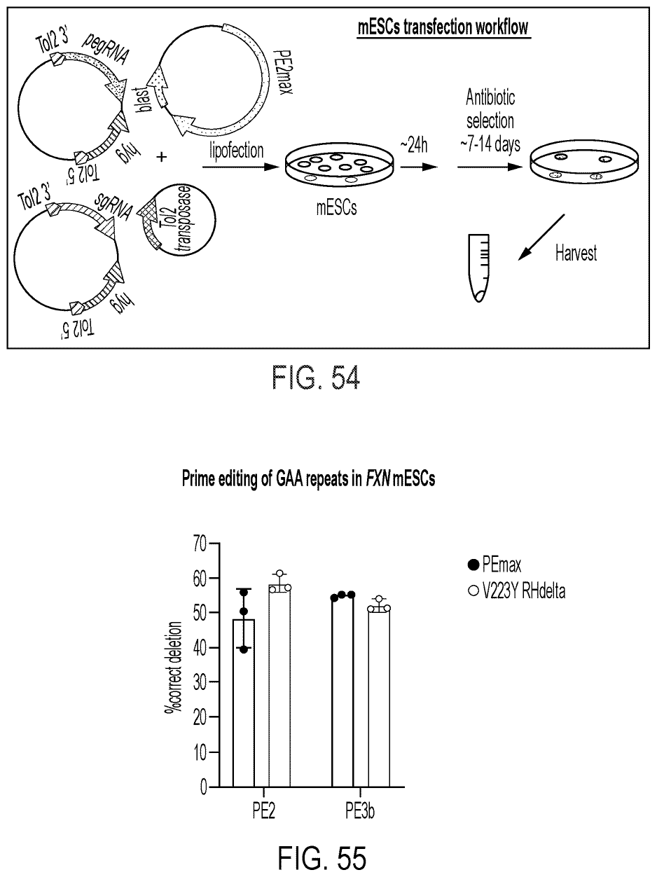

To validate the prime editing strategy in more relevant cell lines, transgenic mouse embryonic stem cell lines (mESCs) containing a ~300 bp fragment of the human FXN intron 1 were generated. Using Tol2 transposase, a cell line expressing FXN intron 1 with 30 GAA repeats was generated. This transgenic cell line facilitated rapid screening of genome editing at non-pathogenic yet larger GAA repeats than at endogenous FXN alleles typically found in wild type human cell lines (typically 8-18 GAAs) (FIGS. 53 and 54). Up to 60% and 55% average prime editing was achieved using all the improvements to the pegRNA (epegRNA, UA scaffold flip) and PE (PEmax, V223Y RHdelta) described above in the PE2 and PE3b system, respectively. This strategy also resulted in 70% precise deletion in transgenic cells (FIGS. 54 and 55).

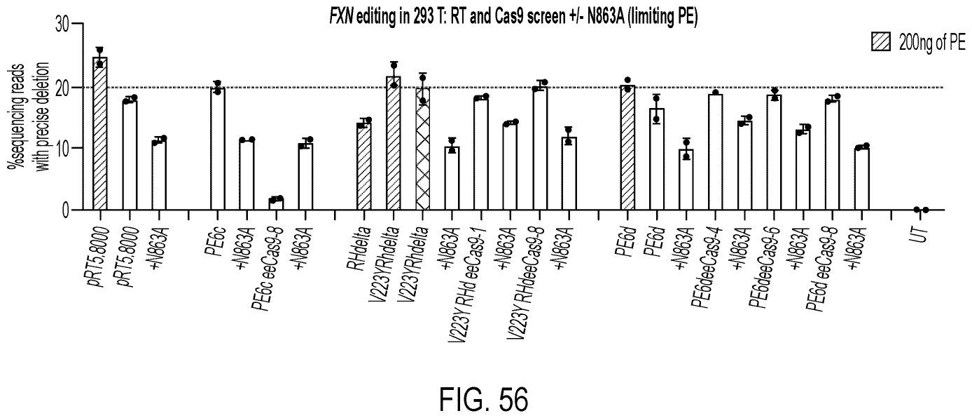

To test additional reverse transcriptase variants and Cas9 candidates that could potentially improve editing efficiency, a number of different editors were screened in the context of limited prime editor quantities to pronounce the differences between the tested editors. The best candidate was found to be a prime editor comprising an MMLV reverse transcriptase with a V223Y mutation and a truncation of the RNaseH domain (e.g., between D497 and I498 of SEQ ID NO: 33) (FIG. 56). A regular dose of prime editor was also transfected for comparison. Standard Cas9 nickase used in PE2 showed equally good editing efficiency as the Cas9-1 and Cas9-8 variants described herein (FIG. 56). Cas9 comprising an N863A mutation abolished editing activity. Cas9-1 and Cas9-8 were included in further optimizations in cell models with pathogenic GAA repeats. PEmax utilizing V223Y RNaseH-truncated MMLV reverse transcriptase or pRT-5.800max (i.e., prime editor comprising Tf1 RT) both outperformed other RT variants in a PE3b system with 3b nicking guide RNA (FIG. 56). Both editors are much smaller than standard PEmax, which provides advantages for delivery and other applications.

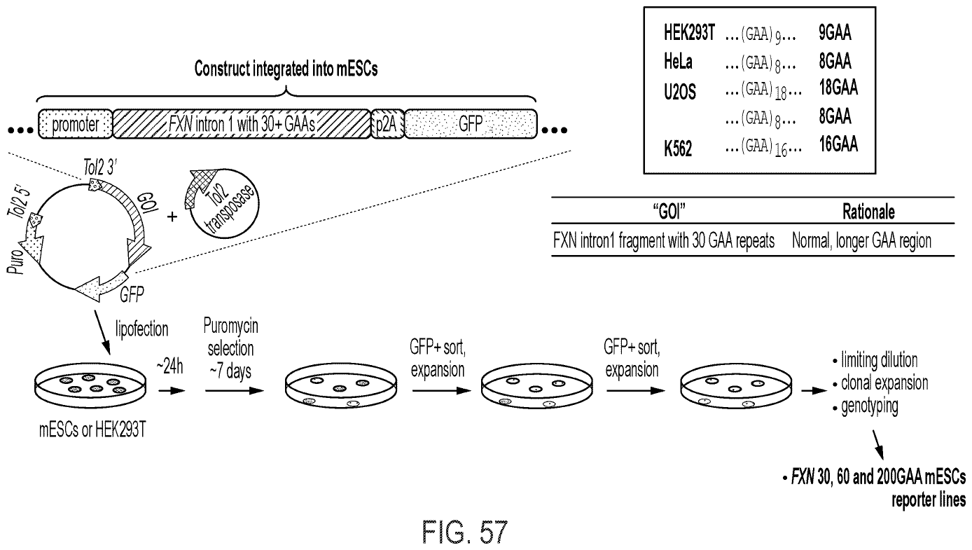

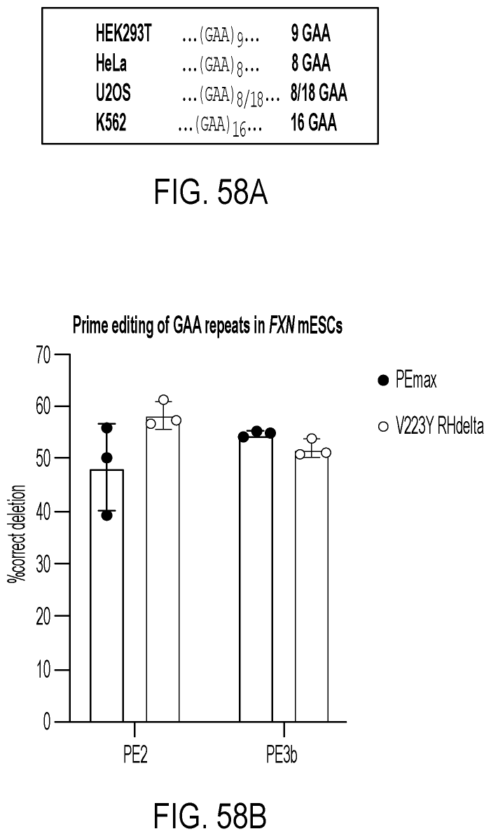

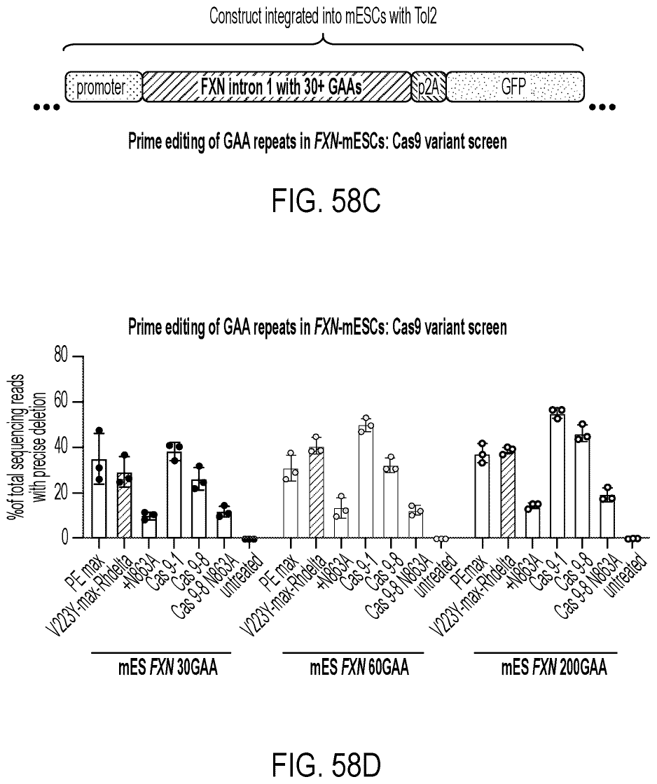

Besides FXN-mESC cells carrying 30 GAA repeats, two additional cell lines with an integrated fragment of FXN intron 1 with 60 or 200 GAA repeats were generated (FIG. 57). The leading prime editing strategies were tested in all three FXN-mESCs lines with successful deletion of the targeted sequence (FIGS. 58A-58D). It was confirmed that standard Cas9 performs very well, but Cas9 variants eeCas9-1 and eeCas9-8 resulted in increased editing efficiency, especially in the context of longer GAA repeats (FIG. 58D).

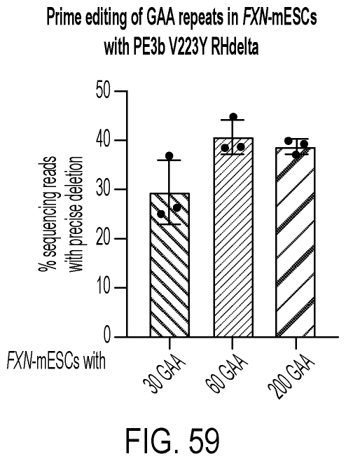

pegRNA with homology of 40 nucleotides resulted in particularly good editing efficiency in FXN mESC cells, and use of an extra nicking guide did not improve editing efficiency further. Additionally, editing of longer alleles (30, 60, and 200 GAAs) was more efficient than editing in HEK293T cells (9GAAs) (FIG. 59). PEmax with V223Y RNaseH-truncated MMLV reverse transcriptase performed similar or better than standard PE2max but has the advantage of being smaller sized (FIG. 58B).

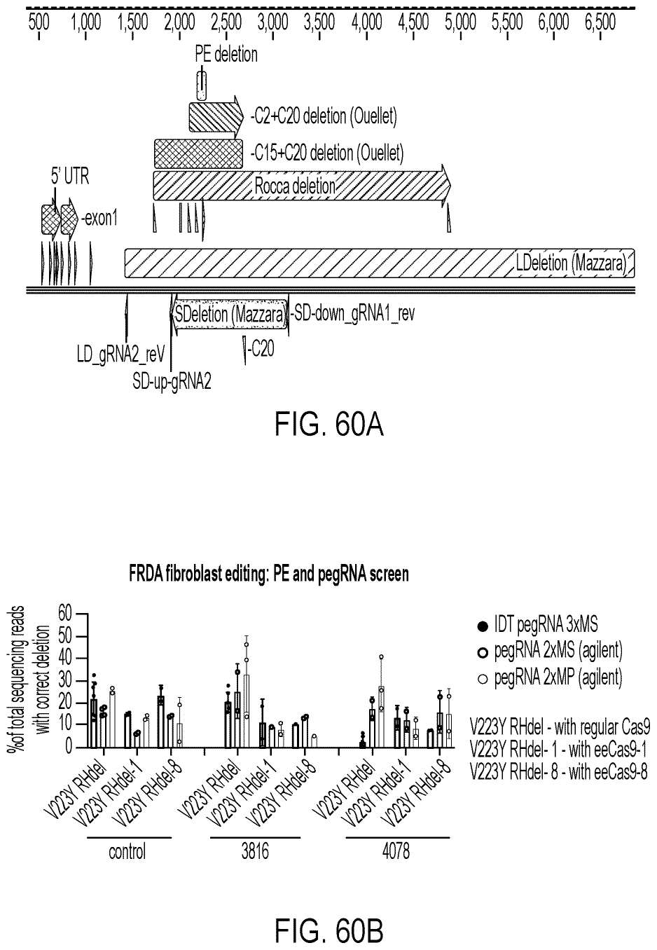

Next, the leading prime editing strategies were tested in FRDA patient-derived fibroblasts (line 3816 and line 4078) and in control fibroblasts with a healthy number of GAA repeats (FIG. 60A). Three different commercially produced synthetic pegRNA with the same sequence but different RNA modifications that influence RNA stability were used, and the results were compared. It was found that across different pegRNA molecules and fibroblast lines, standard Cas9 nickase from PE2 with V223Y RNaseH-truncated MMLV reverse transcriptase performed best (FIG. 60B).

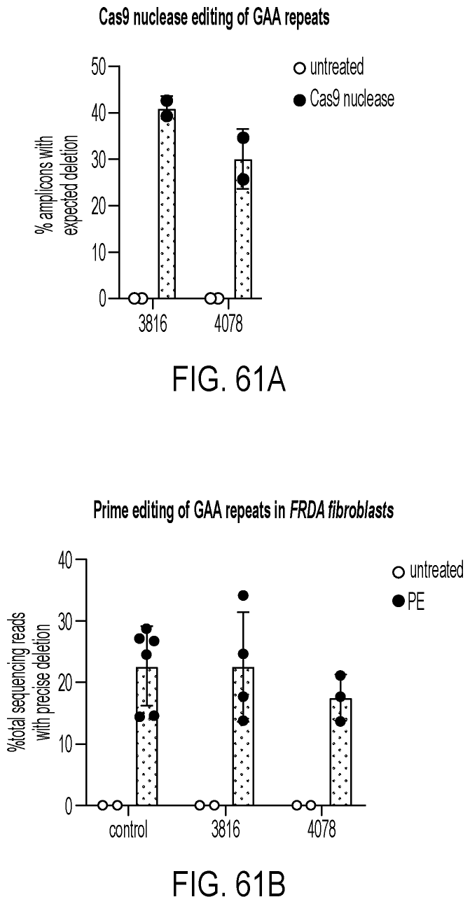

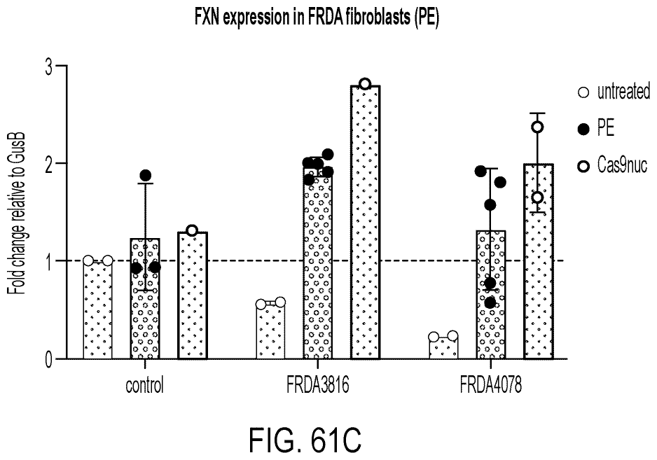

Next, the prime edited fibroblast cells were used to assess phenotypic rescue, as measured by FXN expression (RNA expression measured by ddPCR) (FIGS. 61A-61C). This was found to be highly reduced in FRDA patient-derived cells (FIG. 61C). The prime editing result (FIG. 61B) was compared to rescue in cells edited with a Cas9 nuclease strategy (FIG. 61A), previously published by Rocca et al., Methods and Clinical Development, 2020, 17, 1026-1036. It was found that patient cells treated by prime editing showed a substantial increase in FXN expression, further confirming that prime editing has therapeutic potential for rescuing the main phenotypic trait of FRDA (reduced FXN expression).

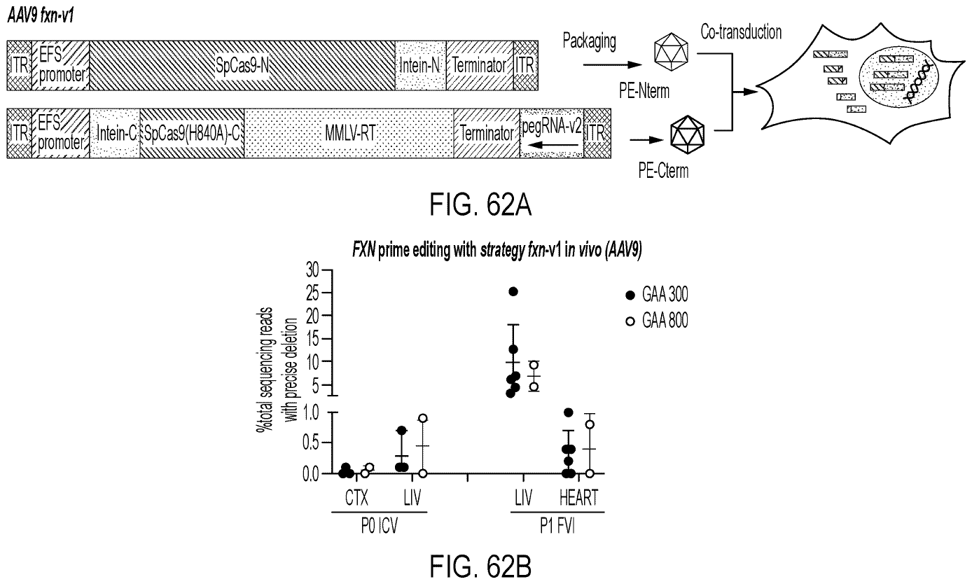

For in vivo studies of prime editing GAA repeats, a YG8s mouse model was used (Virmouni et al., Dis Model Mech, 2015, 8(3), 225-235). Before testing the V223Y RNaseH-truncated MMLV reverse transcriptase prime editor, PEmax, together with the leading pegRNA design (and no nicking guide RNA) was packaged (FIG. 62A) and tested in vivo in two mouse models of FRDA, with either 300 GAAs or 800 GAAs (FIG. 62B). The mice were treated by injecting dual-AAV on postnatal day 0 via ICV (intracerebroventricular injection) or on postnatal day 1 via FVI (facial vein injection). It was determined that AAV9 FXN-v1 strategy shows promising results when delivered systemically. On average, 10% editing in the liver was achieved (FIG. 62B). As the target tissues in FRDA are the CNS and heart, the delivery strategy was further optimized.

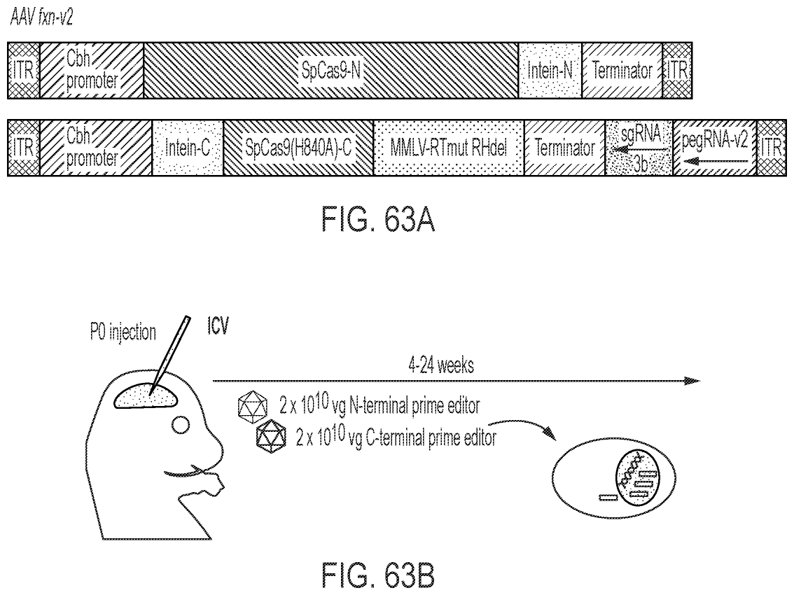

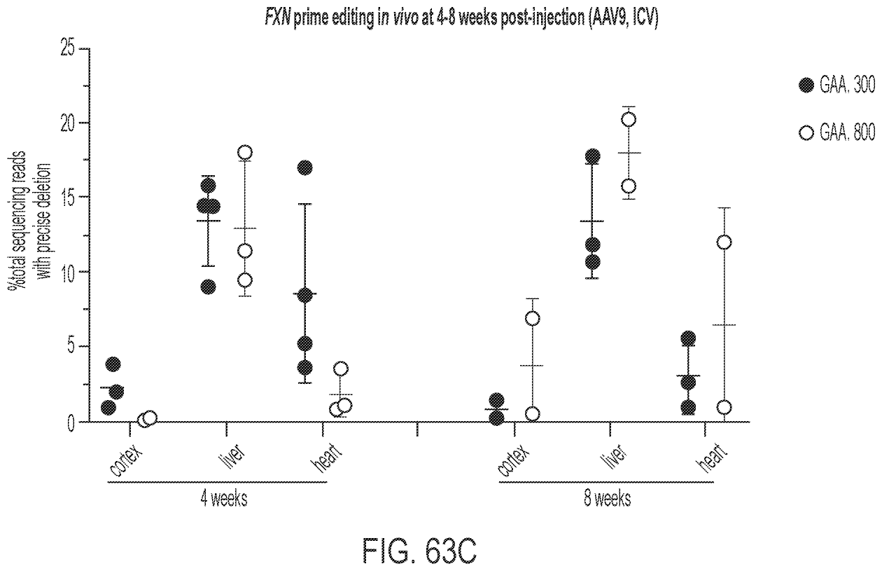

The optimized prime editing strategy included the pegRNA design described herein, PE3b, and PEmax architecture with V223Y RNaseH-truncated MMLV reverse transcriptase. AAV architecture described in Davis et al., NBME, 2023 was also included to optimize delivery (FIG. 63A). Mice were treated with dual-AAV9 by P0 ICV injection (FIG. 63B), and editing was measured across the CNS and systemic tissues after 4 and 8 weeks (FIG. 63C). This improved prime editing AAV delivery strategy (AAV9 FXN-v2; see FIG. 63A) yielded prime editing efficiencies in the liver of approximately 15% and in the heart of approximately 10% and showed non-zero editing in the cortex (FIG. 63C).

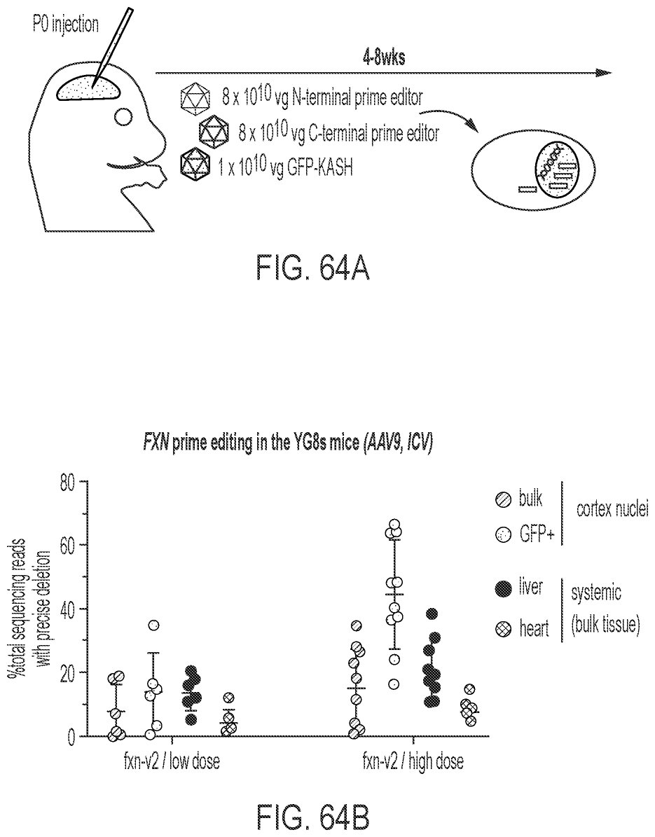

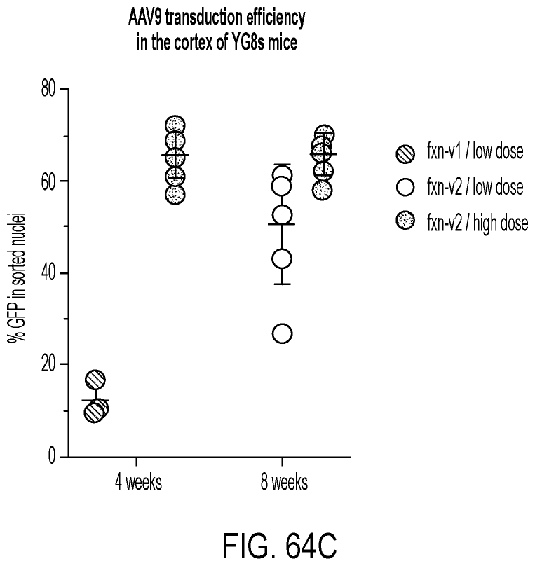

The transduction efficiency of the AAV9 FXN-v2 strategy was then evaluated in the cortex, and editing efficiency was evaluated across different tissues in transduced and bulk nuclei (FIGS. 64A-64C). Additionally, the mice were treated with an increased dose of the AAV9 FXN-v2 to determine the effect of dose on prime editing. The FXN-v2 strategy significantly increased transduction efficiency (FIG. 64C). And the use of a higher viral dose (approximately four times higher) increased the transduction efficiency. No increase in transduction efficiency was observed between four week- and eight week-long treatments. Importantly, it was determined that the higher dose FXN-v2 strategy yielded approximately 20% editing efficiency in bulk cortex nuclei and approximately 40% editing efficiency in transduced cortex nuclei (FIG. 64B). Additionally, editing efficiency in the liver and heart reached approximately 20% and 10%, respectively, after FXN-v2 treatment (FIG. 64B).

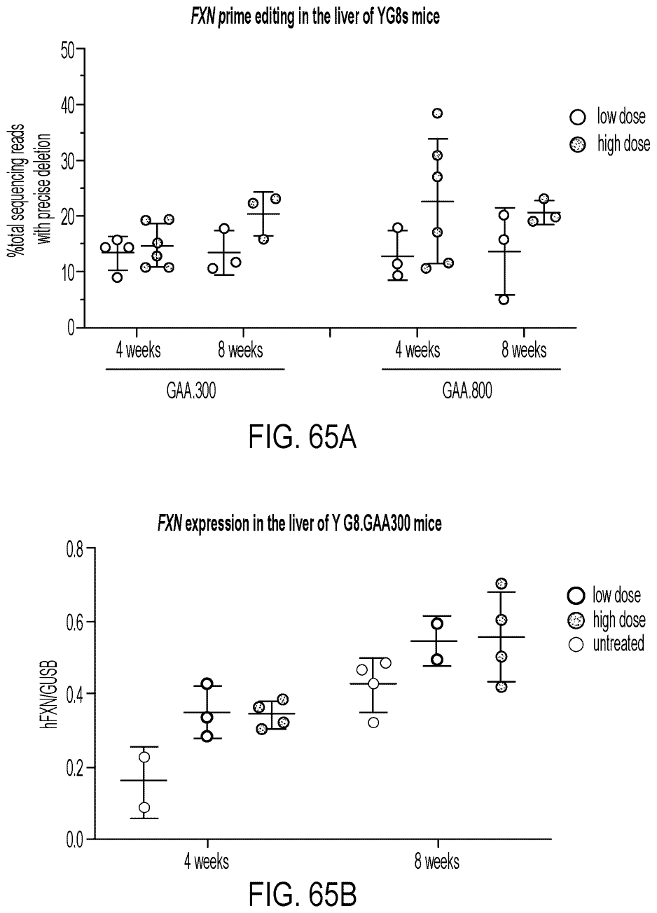

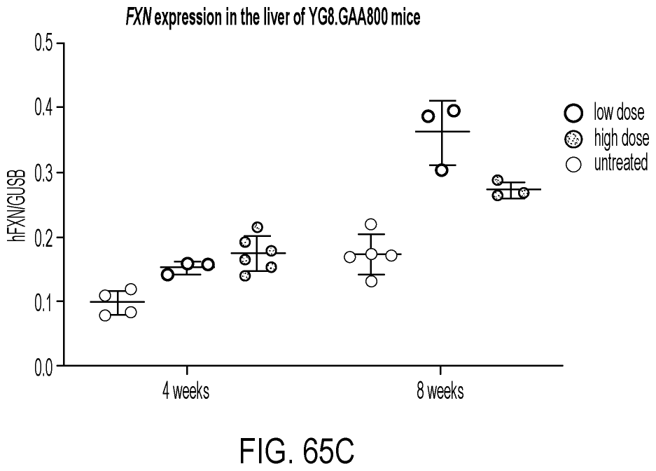

It was then determined whether FRDA mice successfully treated with AAV9 FXN-v2 showed any rescue of FXN expression (FIGS. 65A-65C). Accordingly, FXN expression levels in these mice was assessed by measuring RNA expression by ddPCR in the liver of mice treated with a low or high dose of FXN-v2 for 4 and 8 weeks. Compared to untreated animals, both GAA.300 and GAA.800 mice were found to present an obvious increase in FXN expression that continues to increase over time (FIGS. 65B-65C).

TABLE 1
Sequences used in Example 1

| | SEQ | | | |
|---|---|---|---|---|
| Name of | ID | | | |
| Construct | NO: | Sequence | Description | Notes |
| N2 | 387 | GGCGGCTGAGGAAGCTGAGG | nicking guide | only spacer sequence |
| N3 | 385 | GGCGGCGGCGGCGGCGGTGG | nicking guide | only spacer sequence |
| N5 | 384 | GCTGCTGCTGCTGCTGCTGC | nicking guide | only spacer sequence |
| N9 | 393 | GAAGGACTTGAGGGACTCGA | nicking guide | only spacer sequence |
| N12 | 394 | GTGAGGAAGCTGAGGAGGCGG | nicking guide | only spacer sequence |
| htt_h16_pbs8 | 454 | GACCCTGGAAAAGCTGATGAGTTT | pegRNA for | spacer + scaffold + flap |
| | | TAGAGCTAGAAATAGCAAGTTAAA | HTT, 4CAG | |
| | | ATAAGGCTAGTCCGTTATCAACTT | insertion (6Q) | |
| | | GAAAAAGTGGCACCGAGTCGGTGC | | |
| | | GCGGTGGCGGCTGTTGCTGCTGCT | | |
| | | GCTGGAAGGACTTGAGGGACTCGA | | |
| | | AGGCTTTCATCAGCTTT | | |
| htt_h16_ | 455 | GACCCTGGAAAAGCTGATGAGTTT | pegRNA for | spacer + scaffold + flap |
| pbs10 | | TAGAGCTAGAAATAGCAAGTTAAA | HTT, 4CAG | |
| | | ATAAGGCTAGTCCGTTATCAACTT | insertion (6Q) | |
| | | GAAAAAGTGGCACCGAGTCGGTGC | | |
| | | GCGGTGGCGGCTGTTGCTGCTGCT | | |
| | | GCTGGAAGGACTTGAGGGACTCGA | | |
| | | AGGCTTTCATCAGCTTTTC | | |
| htt_h16_ | 456 | GACCCTGGAAAAGCTGATGAGTTT | pegRNA for | spacer + scaffold + flap |
| pbs12 | | TAGAGCTAGAAATAGCAAGTTAAA | HTT, 4CAG | |
| | | ATAAGGCTAGTCCGTTATCAACTT | insertion (6Q) | |
| | | GAAAAAGTGGCACCGAGTCGGTGC | | |
| | | GCGGTGGCGGCTGTTGCTGCTGCT | | |
| | | GCTGGAAGGACTTGAGGGACTCGA | | |
| | | AGGCTTTCATCAGCTTTTCCA | | |
| htt_h16_ | 457 | GACCCTGGAAAAGCTGATGAGTTT | pegRNA for | spacer + scaffold + flap |
| pbs13 | | TAGAGCTAGAAATAGCAAGTTAAA | HTT, 4CAG | |
| | | ATAAGGCTAGTCCGTTATCAACTT | insertion (6Q) | |
| | | GAAAAAGTGGCACCGAGTCGGTGC | | |
| | | GCGGTGGCGGCTGTTGCTGCTGCT | | |
| | | GCTGGAAGGACTTGAGGGACTCGA | | |
| | | AGGCTTTCATCAGCTTTTCCAG | | |
| htt_h16_ | 458 | GACCCTGGAAAAGCTGATGAGTTT | pegRNA for | spacer + scaffold + flap |
| pbs14 | | TAGAGCTAGAAATAGCAAGTTAAA | HTT, 4CAG | |
| | | ATAAGGCTAGTCCGTTATCAACTT | insertion (6Q) | |
| | | GAAAAAGTGGCACCGAGTCGGTGC | | |
| | | GCGGTGGCGGCTGTTGCTGCTGCT | | |
| | | GCTGGAAGGACTTGAGGGACTCGA | | |
| | | AGGCTTTCATCAGCTTTTCCAGG | | |
| htt_h20_pbs8 | 459 | GACCCTGGAAAAGCTGATGAGTTT | pegRNA for | spacer + scaffold + flap |
| | | TAGAGCTAGAAATAGCAAGTTAAA | HTT, 4CAG | |
| | | ATAAGGCTAGTCCGTTATCAACTT | insertion (6Q) | |
| | | GAAAAAGTGGCACCGAGTCGGTGC | | |
| | | GGCGGCGGTGGCGGCTGTTGCTGC | | |
| | | TGCTGCTGGAAGGACTTGAGGGAC | | |
| | | TCGAAGGCTTTCATCAGCTTT | | |
| htt_h20_pbs9 | 460 | GACCCTGGAAAAGCTGATGAGTTT | pegRNA for | spacer + scaffold + flap |
| | | TAGAGCTAGAAATAGCAAGTTAAA | HTT, 4CAG | |
| | | ATAAGGCTAGTCCGTTATCAACTT | insertion (6Q) | |
| | | GAAAAAGTGGCACCGAGTCGGTGC | | |
| | | GGCGGCGGTGGCGGCTGTTGCTGC | | |
| | | TGCTGCTGGAAGGACTTGAGGGAC | | |
| | | TCGAAGGCTTTCATCAGCTTTT | | |
| htt_h20_ | 461 | GACCCTGGAAAAGCTGATGAGTTT | pegRNA for | spacer + scaffold + flap |
| pbs11 | | TAGAGCTAGAAATAGCAAGTTAAA | HTT, 4CAG | |
| | | ATAAGGCTAGTCCGTTATCAACTT | insertion (6Q) | |
| | | GAAAAAGTGGCACCGAGTCGGTGC | | |
| | | GGCGGCGGTGGCGGCTGTTGCTGC | | |
| | | TGCTGCTGGAAGGACTTGAGGGAC | | |
| | | TCGAAGGCTTTCATCAGCTTTTCC | | |
| htt_h20_ | 462 | GACCCTGGAAAAGCTGATGAGTTT | pegRNA for | spacer + scaffold + flap |
| pbs12 | | TAGAGCTAGAAATAGCAAGTTAAA | HTT, 4CAG | |
| | | ATAAGGCTAGTCCGTTATCAACTT | insertion (6Q) | |
| | | GAAAAAGTGGCACCGAGTCGGTGC | | |
| | | GGCGGCGGTGGCGGCTGTTGCTGC | | |
| | | TGCTGCTGGAAGGACTTGAGGGAC | | |
| | | TCGAAGGCTTTCATCAGCTTTTCCA | | |
| htt_h20_ | 463 | GACCCTGGAAAAGCTGATGAGTTT | pegRNA for | spacer + scaffold + flap |
| pbs13 | | TAGAGCTAGAAATAGCAAGTTAAA | HTT, 4CAG | |
| | | ATAAGGCTAGTCCGTTATCAACTT | insertion (6Q) | |
| | | GAAAAAGTGGCACCGAGTCGGTGC | | |
| | | GGCGGCGGTGGCGGCTGTTGCTGC | | |
| | | TGCTGCTGGAAGGACTTGAGGGAC | | |
| | | TCGAAGGCTTTCATCAGCTTTTCCA | | |
| | | G | | |
| htt_h20_ | 464 | GACCCTGGAAAAGCTGATGAGTTT | pegRNA for | spacer + scaffold + flap |
| pbs14 | | TAGAGCTAGAAATAGCAAGTTAAA | HTT, 4CAG | |
| | | ATAAGGCTAGTCCGTTATCAACTT | insertion (6Q) | |
| | | GAAAAAGTGGCACCGAGTCGGTGC | | |
| | | GGCGGCGGTGGCGGCTGTTGCTGC | | |
| | | TGCTGCTGGAAGGACTTGAGGGAC | | |
| | | TCGAAGGCTTTCATCAGCTTTTCCA | | |
| | | GG | | |
| htt_h25_pbs8 | 465 | GACCCTGGAAAAGCTGATGAGTTT | pegRNA for | spacer + scaffold + flap |
| | | TAGAGCTAGAAATAGCAAGTTAAA | HTT, 4CAG | |
| | | ATAAGGCTAGTCCGTTATCAACTT | insertion (6Q) | |
| | | GAAAAAGTGGCACCGAGTCGGTGC | | |
| | | GCGGCGGCGGCGGTGGCGGCTGTT | | |
| | | GCTGCTGCTGCTGGAAGGACTTGA | | |
| | | GGGACTCGAAGGCTTTCATCAGCT | | |
| | | TT | | |
| htt_h25_pbs9 | 466 | GACCCTGGAAAAGCTGATGAGTTT | pegRNA for | spacer + scaffold + flap |
| | | TAGAGCTAGAAATAGCAAGTTAAA | HTT, 4CAG | |
| | | ATAAGGCTAGTCCGTTATCAACTT | insertion (6Q) | |
| | | GAAAAAGTGGCACCGAGTCGGTGC | | |
| | | GCGGCGGCGGCGGTGGCGGCTGTT | | |
| | | GCTGCTGCTGCTGGAAGGACTTGA | | |
| | | GGGACTCGAAGGCTTTCATCAGCT | | |
| | | TTT | | |
| htt_h25_ | 467 | GACCCTGGAAAAGCTGATGAGTTT | pegRNA for | spacer + scaffold + flap |
| pbs10 | | TAGAGCTAGAAATAGCAAGTTAAA | HTT, 4CAG | |
| | | ATAAGGCTAGTCCGTTATCAACTT | insertion (6Q) | |
| | | GAAAAAGTGGCACCGAGTCGGTGC | | |
| | | GCGGCGGCGGCGGTGGCGGCTGTT | | |
| | | GCTGCTGCTGCTGGAAGGACTTGA | | |
| | | GGGACTCGAAGGCTTTCATCAGCT | | |
| | | TTTC | | |
| htt_h25_ | 468 | GACCCTGGAAAAGCTGATGAGTTT | pegRNA for | spacer + scaffold + flap |
| pbs11 | | TAGAGCTAGAAATAGCAAGTTAAA | HTT, 4CAG | |
| | | ATAAGGCTAGTCCGTTATCAACTT | insertion (6Q) | |
| | | GAAAAAGTGGCACCGAGTCGGTGC | | |
| | | GCGGCGGCGGCGGTGGCGGCTGTT | | |
| | | GCTGCTGCTGCTGGAAGGACTTGA | | |
| | | GGGACTCGAAGGCTTTCATCAGCT | | |
| | | TTTCC | | |
| htt_h25_ | 469 | GACCCTGGAAAAGCTGATGAGTTT | pegRNA for | spacer + scaffold + flap |
| pbs12 | | TAGAGCTAGAAATAGCAAGTTAAA | HTT, 4CAG | |
| | | ATAAGGCTAGTCCGTTATCAACTT | insertion (6Q) | |
| | | GAAAAAGTGGCACCGAGTCGGTGC | | |
| | | GCGGCGGCGGCGGTGGCGGCTGTT | | |
| | | GCTGCTGCTGCTGGAAGGACTTGA | | |
| | | GGGACTCGAAGGCTTTCATCAGCT | | |
| | | TTTCCA | | |
| htt_h25_ | 470 | GACCCTGGAAAAGCTGATGAGTTT | pegRNA for | spacer + scaffold + flap |
| pbs13 | | TAGAGCTAGAAATAGCAAGTTAAA | HTT, 4CAG | |
| | | ATAAGGCTAGTCCGTTATCAACT | insertion (6Q) | |
| | | GAAAAAGTGGCACCGAGTCGGTGC | | |
| | | GCGGCGGCGGCGGTGGCGGCTGTT | | |
| | | GCTGCTGCTGCTGGAAGGACTTGA | | |
| | | GGGACTCGAAGGCTTTCATCAGCT | | |
| | | TTTCCAG | | |
| htt_h25_ | 471 | GACCCTGGAAAAGCTGATGAGTTT | pegRNA for | spacer + scaffold + flap |
| pbs14 | | TAGAGCTAGAAATAGCAAGTTAAA | HTT, 4CAG | |
| | | ATAAGGCTAGTCCGTTATCAACTT | insertion (6Q) | |
| | | GAAAAAGTGGCACCGAGTCGGTGC | | |
| | | GCGGCGGCGGCGGTGGCGGCTGTT | | |
| | | GCTGCTGCTGCTGGAAGGACTTGA | | |
| | | GGGACTCGAAGGCTTTCATCAGCT | | |
| | | TTTCCAGG | | |
| htt_h31_pbs9 | 472 | GACCCTGGAAAAGCTGATGAGTTT | pegRNA for | spacer + scaffold + flap |
| | | TAGAGCTAGAAATAGCAAGTTAAA | HTT, 4CAG | |
| | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGGAAG | | |
| | | GACTTGAGGGACTCGAAGGCTTTC | | |
| | | ATCAGCTTTT | | |
| htt_h31_ | 473 | GACCCTGGAAAAGCTGATGAGTTT | pegRNA for | spacer + scaffold + flap |
| pbs10 | | TAGAGCTAGAAATAGCAAGTTAAA | HTT, 4CAG | |
| | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGGAAG | | |
| | | GACTTGAGGGACTCGAAGGCTTTC | | |
| | | ATCAGCTTTTC | | |
| htt_h31_ | 474 | GACCCTGGAAAAGCTGATGAGTTT | pegRNA for | spacer + scaffold + flap |
| pbs12 | | TAGAGCTAGAAATAGCAAGTTAAA | HTT, 4CAG | |
| | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGGAAG | | |
| | | GACTTGAGGGACTCGAAGGCTTTC | | |
| | | ATCAGCTTTTCCA | | |
| htt_h31_ | 475 | GACCCTGGAAAAGCTGATGAGTTT | pegRNA for | spacer + scaffold + flap |
| pbs13 | | TAGAGCTAGAAATAGCAAGTTAAA | HTT, 4CAG | |
| | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGGAAG | | |
| | | GACTTGAGGGACTCGAAGGCTTTC | | |
| | | ATCAGCTTTTCCAG | | |
| htt_h35_pbs8 | 476 | GACCCTGGAAAAGCTGATGAGTTT | pegRNA for | spacer + scaffold + flap |
| | | TAGAGCTAGAAATAGCAAGTTAAA | HTT, 4CAG | |
| | | ATAAGGCTAGTCCGTTATCAACTT | insertion (6Q) | |
| | | GAAAAAGTGGCACCGAGTCGGTGC | | |
| | | GGCGGCGGCGGCGGCGGCGGCGG | | |
| | | TGGCGGCTGTTGCTGCTGCTGCTG | | |
| | | GAAGGACTTGAGGGACTCGAAGG | | |
| | | CTTTCATCAGCTTT | | |
| htt_h35_ | 477 | GACCCTGGAAAAGCTGATGAGTTT | pegRNA for | spacer + scaffold + flap |
| pbs10 | | TAGAGCTAGAAATAGCAAGTTAAA | HTT, 4CAG | |
| | | ATAAGGCTAGTCCGTTATCAACTT | insertion (6Q) | |
| | | GAAAAAGTGGCACCGAGTCGGTGC | | |
| | | GGCGGCGGCGGCGGCGGCGGCGG | | |
| | | TGGCGGCTGTTGCTGCTGCTGCTG | | |
| | | GAAGGACTTGAGGGACTCGAAGG | | |
| | | CTTTCATCAGCTTTTC | | |
| htt_h31_ | 478 | GACCCTGGAAAAGCTGATGAGTTT | epegRNA with | spacer + scaffold + |
| pbs10eq1 | | TAGAGCTAGAAATAGCAAGTTAAA | rational linker, | flap + linker + 3′motif |
| | | ATAAGGCTAGTCCGTTATCAACTT | 4CAG | |
| | | GAAAAAGTGGCACCGAGTCGGTGC | insertion (6Q) | |
| | | GCGGCGGCGGCGGCGGCGGTGGC | | |
| | | GGCTGTTGCTGCTGCTGCTGGAAG | | |
| | | GACTTGAGGGACTCGAAGGCTTTC | | |
| | | ATCAGCTTTTCCAATCACTCGCGGT | | |
| | | TCTATCTAGTTACGCGTTAAACCA | | |
| | | ACTAGAA | | |
| htt_h31_ | 479 | GACCCTGGAAAAGCTGATGAGTTT | epegRNA with | spacer + scaffold + |
| pbs10eq1A | | TAGAGCTAGAAATAGCAAGTTAAA | pegLIT linker, | flap + linker + 3′motif |
| | | ATAAGGCTAGTCCGTTATCAACTT | 4CAG | |
| | | GAAAAAGTGGCACCGAGTCGGTGC | insertion (6Q) | |
| | | GCGGCGGCGGCGGCGGCGGTGGC | | |
| | | GGCTGTTGCTGCTGCTGCTGGAAG | | |
| | | GACTTGAGGGACTCGAAGGCTTTC | | |
| | | ATCAGCTTTTCGTATAATACGCGG | | |
| | | TTCTATCTAGTTACGCGTTAAACCA | | |
| | | ACTAGAA | | |
| htt_h40_ | 480 | GACCCTGGAAAAGCTGATGAGTTT | epegRNA with | spacer + scaffold + |
| pbs10eq1A | | TAGAGCTAGAAATAGCAAGTTAAA | pegLIT linker, | flap + linker + 3′motif |
| | | ATAAGGCTAGTCCGTTATCAACTT | 4CAG | |
| | | GAAAAAGTGGCACCGAGTCGGTGC | insertion (6Q) | |
| | | GAGGAGGCGGCGGCGGCGGCGGC | | |
| | | GGCGGTGGCGGCTGTTGCTGCTGC | | |
| | | TGCTGGAAGGACTTGAGGGACTCG | | |
| | | AAGGCTTTCATCAGCTTTTCATTAA | | |
| | | ATTCGCGGTTCTATCTAGTTACGCG | | |
| | | TTAAACCAACTAGAA | | |
| htt_h31_ | 481 | GACCCTGGAAAAGCTGATGAGTTT | pegRNA for | spacer + scaffold + |
| pbs10mpknot | | TAGAGCTAGAAATAGCAAGTTAAA | HTT with | flap + linker + 3′motif |
| | | ATAAGGCTAGTCCGTTATCAACTT | mpknot | |
| | | GAAAAAGTGGCACCGAGTCGGTGC | modification, | |
| | | GCGGCGGCGGCGGCGGCGGTGGC | 4CAG | |
| | | GGCTGTTGCTGCTGCTGCTGGAAG | insertion (6Q) | |
| | | GACTTGAGGGACTCGAAGGCTTTC | | |
| | | ATCAGCTTTTCCAATCACTGGGTC | | |
| | | AGGAGCCCCCCCCCTGAACCCAGG | | |
| | | ATAACCCTCAAAGTCGGGGGGC | | |
| htt_h40_ | 482 | GACCCTGGAAAAGCTGATGAGTTT | pegRNA for | spacer + scaffold + |
| pbs10mpknot | | TAGAGCTAGAAATAGCAAGTTAAA | HTT with | flap + linker + 3′motif |
| | | ATAAGGCTAGTCCGTTATCAACTT | mpknot | |
| | | GAAAAAGTGGCACCGAGTCGGTGC | modification, | |
| | | GAGGAGGCGGCGGCGGCGGCGGC | 4CAG | |
| | | GGCGGTGGCGGCTGTTGCTGCTGC | insertion (6Q) | |
| | | TGCTGGAAGGACTTGAGGGACTCG | | |
| | | AAGGCTTTCATCAGCTTTTCCAATC | | |
| | | ACTGGGTCAGGAGCCCCCCCCCTG | | |
| | | AACCCAGGATAACCCTCAAAGTCG | | |
| | | GGGGGC | | |
| htt_h25_ | 483 | GACCCTGGAAAAGCTGATGAGTTT | epegRNA with | spacer + scaffold + |
| pbs10eq1 | | TAGAGCTAGAAATAGCAAGTTAAA | rational linker, | flap + linker + 3′motif |
| | | ATAAGGCTAGTCCGTTATCAACTT | 4CAG | |
| | | GAAAAAGTGGCACCGAGTCGGTGC | insertion (6Q) | |
| | | GCGGCGGCGGCGGTGGCGGCTGTT | | |
| | | GCTGCTGCTGCTGGAAGGACTTGA | | |
| | | GGGACTCGAAGGCTTTCATCAGCT | | |
| | | TTTCCAATCACTCGCGGTTCTATCT | | |
| | | AGTTACGCGTTAAACCAACTAGAA | | |
| htt_h35_ | 484 | GACCCTGGAAAAGCTGATGAGTTT | epegRNA with | spacer + scaffold + |
| pbs10eq1 | | TAGAGCTAGAAATAGCAAGTTAAA | rational linker, | flap + linker + 3′motif |
| | | ATAAGGCTAGTCCGTTATCAACTT | insertion (6Q) | |
| | | GAAAAAGTGGCACCGAGTCGGTGC | 4CAG | |
| | | GGCGGCGGCGGCGGCGGCGGCGG | | |
| | | TGGCGGCTGTTGCTGCTGCTGCTG | | |
| | | GAAGGACTTGAGGGACTCGAAGG | | |
| | | CTTTCATCAGCTTTTCCAATCACTC | | |
| | | GCGGTTCTATCTAGTTACGCGTTA | | |
| | | AACCAACTAGAA | | |
| htt_h40_ | 485 | GACCCTGGAAAAGCTGATGAGTTT | epegRNA with | spacer + scaffold + |
| pbs10eq1 | | TAGAGCTAGAAATAGCAAGTTAAA | rational linker, | flap + linker + 3′motif |
| | | ATAAGGCTAGTCCGTTATCAACTT | 4CAG | |
| | | GAAAAAGTGGCACCGAGTCGGTGC | insertion (6Q) | |
| | | GAGGAGGCGGCGGCGGCGGCGGC | | |
| | | GGCGGTGGCGGCTGTTGCTGCTGC | | |
| | | TGCTGGAAGGACTTGAGGGACTCG | | |
| | | AAGGCTTTCATCAGCTTTTCCAATC | | |
| | | ACTCGCGGTTCTATCTAGTTACGC | | |
| | | GTTAAACCAACTAGAA | | |
| htt_Pdel_f1_ | 486 | GACCCTGGAAAAGCTGATGAGTTT | epegRNA for | spacer + scaffold + |
| r1_p10eq1 | | TAGAGCTAGAAATAGCAAGTTAAA | dual pegRNA | flap + linker + 3′motif |
| | | ATAAGGCTAGTCCGTTATCAACTT | approach, | |
| | | GAAAAAGTGGCACCGAGTCGGTGC | 4CAG | |
| | | GCGGCGGCGGCTGAGGAAGCTGA | insertion (6Q) | |
| | | GGAGGCGGCGGCGGCGGCGGCGG | | |
| | | CGGTGGCGGCTGTTGCTGCTGCTG | | |
| | | CTGGAAGGACTTGAGGGACTCGAA | | |
| | | GGCTTTCATCAGCTTTTCCAATCAC | | |
| | | TCGCGGTTCTATCTAGTTACGCGTT | | |
| | | AAACCAACTAGAA | | |
| htt_Pdel_f1_ | 487 | GACCCTGGAAAAGCTGATGAGTTT | epegRNA for | spacer + scaffold + |
| r2_p10eq1 | | TAGAGCTAGAAATAGCAAGTTAAA | dual pegRNA | flap + linker + 3′motif |
| | | ATAAGGCTAGTCCGTTATCAACTT | approach, | |
| | | GAAAAAGTGGCACCGAGTCGGTGC | 4CAG | |
| | | AGCTGAGGAGGCGGCGGCGGCGG | insertion (6Q) | |
| | | CGGCGGCGGTGGCGGCTGTTGCTG | | |
| | | CTGCTGCTGGAAGGACTTGAGGGA | | |
| | | CTCGAAGGCTTTCATCAGCTTTTCC | | |
| | | AATCACTCGCGGTTCTATCTAGTTA | | |
| | | CGCGTTAAACCAACTAGAA | | |
| htt_Pdel_r1_ | 488 | GTGAGGAAGCTGAGGAGGCGGGT | epegRNA for | spacer + scaffold + |
| f1_p10eq1 | | TTTAGAGCTAGAAATAGCAAGTTA | dual pegRNA | flap + linker + 3′motif |
| | | AAATAAGGCTAGTCCGTTATCAAC | approach, | |
| | | TTGAAAAAGTGGCACCGAGTCGGT | 4CAG | |
| | | GCAGACCGCCATGGCGACCCTGGA | insertion (6Q) | |
| | | AAAGCTGATGAAAGCCTTCGAGTC | | |
| | | CCTCAAGTCCTTCCAGCAGCAGCA | | |
| | | GCAACAGCCGCCACCGCCGCCGCC | | |
| | | GCCGCCGCCGCCTCCTCAGCCAAT | | |
| | | CACTCGCGGTTCTATCTAGTTACGC | | |
| | | GTTAAACCAACTAGAA | | |
| htt_Pdel_r2_ | 489 | GGCGGCGGCGGCGGCGGTGGGTTT | epegRNA for | spacer + scaffold + |
| f1_p10eq1 | | TAGAGCTAGAAATAGCAAGTTAAA | dual pegRNA | flap + linker + 3′motif |
| | | ATAAGGCTAGTCCGTTATCAACTT | approach, | |
| | | GAAAAAGTGGCACCGAGTCGGTGC | 4CAG | |
| | | AGACCGCCATGGCGACCCTGGAAA | insertion (6Q) | |
| | | AGCTGATGAAAGCCTTCGAGTCCC | | |
| | | TCAAGTCCTTCCAGCAGCAGCAGC | | |
| | | AACAGCCGCCACCGCCGCCGCCAA | | |
| | | TCACTCGCGGTTCTATCTAGTTACG | | |
| | | CGTTAAACCAACTAGAA | | |
| htt_dflapv2_ | 484 | GACCCTGGAAAAGCTGATGAGTTT | epegRNA for | spacer + scaffold + |
| f1_r1_p10eq1 | | TAGAGCTAGAAATAGCAAGTTAAA | dual pegRNA | flap + linker + 3′motif |
| | | ATAAGGCTAGTCCGTTATCAACTT | approach, | |
| | | GAAAAAGTGGCACCGAGTCGGTGC | 4CAG | |
| | | GGCGGCGGCGGCGGCGGCGGCGG | insertion (6Q) | |
| | | TGGCGGCTGTTGCTGCTGCTGCTG | | |
| | | GAAGGACTTGAGGGACTCGAAGG | | |
| | | CTTTCATCAGCTTTTCCAATCACTC | | |
| | | GCGGTTCTATCTAGTTACGCGTTA | | |
| | | AACCAACTAGAA | | |
| htt_dflapv2_ | 490 | GACCCTGGAAAAGCTGATGAGTTT | epegRNA for | spacer + scaffold + |
| f1_r2_p10eq1 | | TAGAGCTAGAAATAGCAAGTTAAA | dual pegRNA | flap + linker + 3′motif |
| | | ATAAGGCTAGTCCGTTATCAACTT | approach, | |
| | | GAAAAAGTGGCACCGAGTCGGTGC | 4CAG | |
| | | TGGCGGCTGTTGCTGCTGCTGCTG | insertion (6Q) | |
| | | GAAGGACTTGAGGGACTCGAAGG | | |
| | | CTTTCATCAGCTTTTCCAATCACTC | | |
| | | GCGGTTCTATCTAGTTACGCGTTA | | |
| | | AACCAACTAGAA | | |
| htt_dflapv2_ | 491 | GTGAGGAAGCTGAGGAGGCGGGT | epegRNA for | spacer + scaffold + |
| r1_f1_p10eq1 | | TTTAGAGCTAGAAATAGCAAGTTA | dual pegRNA | flap + linker + 3′motif |
| | | AAATAAGGCTAGTCCGTTATCAAC | approach, | |
| | | TTGAAAAAGTGGCACCGAGTCGGT | 4CAG | |
| | | GCTGAAAGCCTTCGAGTCCCTCAA | insertion (6Q) | |
| | | GTCCTTCCAGCAGCAGCAGCAACA | | |
| | | GCCGCCACCGCCGCCGCCGCCGCC | | |
| | | GCCGCCTCCTCAGCCAATCACTCG | | |
| | | CGGTTCTATCTAGTTACGCGTTAA | | |
| | | ACCAACTAGAA | | |
| htt_dflapv2_ | 492 | GGCGGCGGCGGCGGCGGTGGGTTT | epegRNA for | spacer + scaffold + |
| r2_f1_p10eq1 | | TAGAGCTAGAAATAGCAAGTTAAA | dual pegRNA | flap + linker + 3′motif |
| | | ATAAGGCTAGTCCGTTATCAACTT | approach, | |
| | | GAAAAAGTGGCACCGAGTCGGTGC | 4CAG | |
| | | TGAAAGCCTTCGAGTCCCTCAAGT | insertion (6Q) | |
| | | CCTTCCAGCAGCAGCAGCAACAGC | | |
| | | CGCCACCGCCGCCGCCAATCACTC | | |
| | | GCGGTTCTATCTAGTTACGCGTTA | | |
| | | AACCAACTAGAA | | |
| htt_dflapv2_ | 493 | GACCCTGGAAAAGCTGATGAGTTT | epegRNA for | spacer + scaffold + |
| recA_f1_r1_ | | TAGAGCTAGAAATAGCAAGTTAAA | dual pegRNA | flap + linker + 3′motif |
| p8eq1 | | ATAAGGCTAGTCCGTTATCAACTT | approach, | |
| | | GAAAAAGTGGCACCGAGTCGGTGC | 4CAG | |
| | | GGTGGAGGGGGCGGTGGAGGGGG | insertion (6Q) | |
| | | TGGAGGCTGTTGCTGTTGCTGCTG | | |
| | | AAATGATTTCAGTGATTCAAATGC | | |
| | | TTTCATCAGCTTTCAATCACTCGCG | | |
| | | GTTCTATCTAGTTACGCGTTAAACC | | |
| | | AACTAGAA | | |
| htt_dflapv2_ | 494 | GACCCTGGAAAAGCTGATGAGTTT | epegRNA for | spacer + scaffold + |
| recA_f1_r1_ | | TAGAGCTAGAAATAGCAAGTTAAA | dual pegRNA | flap + linker + 3′motif |
| p10eq1 | | ATAAGGCTAGTCCGTTATCAACTT | approach, | |
| | | GAAAAAGTGGCACCGAGTCGGTGC | 4CAG | |
| | | GGTGGAGGGGGCGGTGGAGGGGG | insertion (6Q) | |
| | | TGGAGGCTGTTGCTGTTGCTGCTG | | |
| | | AAATGATTTCAGTGATTCAAATGC | | |
| | | TTTCATCAGCTTTTCCAATCACTCG | | |
| | | CGGTTCTATCTAGTTACGCGTTAA | | |
| | | ACCAACTAGAA | | |
| htt_dflapv2_ | 495 | GACCCTGGAAAAGCTGATGAGTTT | epegRNA for | spacer + scaffold + |
| recA_f1_r2_ | | TAGAGCTAGAAATAGCAAGTTAAA | dual pegRNA | flap + linker + 3′motif |
| p8eq1 | | ATAAGGCTAGTCCGTTATCAACTT | approach, | |
| | | GAAAAAGTGGCACCGAGTCGGTGC | 4CAG | |
| | | TGGAGGCTGTTGCTGTTGCTGCTG | insertion (6Q) | |
| | | AAATGATTTCAGTGATTCAAATGC | | |
| | | TTTCATCAGCTTTCAATCACTCGCG | | |
| | | GTTCTATCTAGTTACGCGTTAAACC | | |
| | | AACTAGAA | | |
| htt_dflapv2_ | 496 | GACCCTGGAAAAGCTGATGAGTTT | epegRNA for | spacer + scaffold + |
| recA_f1_r2_ | | TAGAGCTAGAAATAGCAAGTTAAA | dual pegRNA | flap + linker + 3′motif |
| p10eq1 | | ATAAGGCTAGTCCGTTATCAACTT | approach, | |
| | | GAAAAAGTGGCACCGAGTCGGTGC | 4CAG | |
| | | TGGAGGCTGTTGCTGTTGCTGCTG | insertion (6Q) | |
| | | AAATGATTTCAGTGATTCAAATGC | | |
| | | TTTCATCAGCTTTTCCAATCACTCG | | |
| | | CGGTTCTATCTAGTTACGCGTTAA | | |
| | | ACCAACTAGAA | | |
| htt_dflapv2_ | 497 | GTGAGGAAGCTGAGGAGGCGGGT | epegRNA for | spacer + scaffold + |
| recA_r1_f1_ | | TTTAGAGCTAGAAATAGCAAGTTA | dual pegRNA | flap + linker + 3′motif |
| p8eq1 | | AAATAAGGCTAGTCCGTTATCAAC | approach, | |
| | | TTGAAAAAGTGGCACCGAGTCGGT | 4CAG | |
| | | GCTGAAAGCATTTGAATCACTGAA | insertion (6Q) | |
| | | ATCATTTCAGCAGCAACAGCAACA | | |
| | | GCCTCCACCCCCTCCACCGCCCCCT | | |
| | | CCACCTCCTCACAATCACTCGCGG | | |
| | | TTCTATCTAGTTACGCGTTAAACCA | | |
| | | ACTAGAA | | |
| htt_dflapv2_ | 498 | GTGAGGAAGCTGAGGAGGCGGGT | epegRNA for | spacer + scaffold + |
| recA_r1_f1_ | | TTTAGAGCTAGAAATAGCAAGTTA | dual pegRNA | flap + linker + 3′motif |
| p10eq1 | | AAATAAGGCTAGTCCGTTATCAAC | approach, | |
| | | TTGAAAAAGTGGCACCGAGTCGGT | 4CAG | |
| | | GCTGAAAGCATTTGAATCACTGAA | insertion (6Q) | |
| | | ATCATTTCAGCAGCAACAGCAACA | | |
| | | GCCTCCACCCCCTCCACCGCCCCCT | | |
| | | CCACCTCCTCAGCCAATCACTCGC | | |
| | | GGTTCTATCTAGTTACGCGTTAAA | | |
| | | CCAACTAGAA | | |
| htt_dflapv2_ | 499 | GGCGGCGGCGGCGGCGGTGGGTTT | epegRNA for | spacer + scaffold + |
| recA_r2_f1_ | | TAGAGCTAGAAATAGCAAGTTAAA | dual pegRNA | flap + linker + 3′motif |
| p8eq1 | | ATAAGGCTAGTCCGTTATCAACTT | approach, | |
| | | GAAAAAGTGGCACCGAGTCGGTGC | 4CAG | |
| | | TGAAAGCATTTGAATCACTGAAAT | insertion (6Q) | |
| | | CATTTCAGCAGCAACAGCAACAGC | | |
| | | CTCCACCGCCGCCCAATCACTCGC | | |
| | | GGTTCTATCTAGTTACGCGTTAAA | | |
| | | CCAACTAGAA | | |
| htt_dflapv2_ | 500 | GGCGGCGGCGGCGGCGGTGGGTTT | epegRNA for | spacer + scaffold + |
| recA_r2_f1_ | | TAGAGCTAGAAATAGCAAGTTAAA | dual pegRNA | flap + linker + 3′motif |
| p10eq1 | | ATAAGGCTAGTCCGTTATCAACTT | approach, | |
| | | GAAAAAGTGGCACCGAGTCGGTGC | 4CAG | |
| | | TGAAAGCATTTGAATCACTGAAAT | insertion (6Q) | |
| | | CATTTCAGCAGCAACAGCAACAGC | | |
| | | CTCCACCGCCGCCGCCAATCACTC | | |
| | | GCGGTTCTATCTAGTTACGCGTTA | | |
| | | AACCAACTAGAA | | |
| htt_h31_ | 501 | GACCCTGGAAAAGCTGATGAGTTT | epegRNA for | spacer + scaffold + |
| p10eq1_2CAG | | TAGAGCTAGAAATAGCAAGTTAAA | various CAG | flap + linker + 3′motif |
| | | ATAAGGCTAGTCCGTTATCAACTT | replacement | |
| | | GAAAAAGTGGCACCGAGTCGGTGC | | |
| | | GCGGCGGCGGCGGCGGCGGTGGC | | |
| | | GGCTGTTGCTGCTGGAAGGACTTG | | |
| | | AGGGACTCGAAGGCTTTCATCAGC | | |
| | | TTTTCCAATCACTCGCGGTTCTATC | | |
| | | TAGTTACGCGTTAAACCAACTAGA | | |
| | | A | | |
| htt_h31_ | 502 | GACCCTGGAAAAGCTGATGAGTTT | epegRNA for | spacer + scaffold + |
| p10eq1_3CAG | | TAGAGCTAGAAATAGCAAGTTAAA | various CAG | flap + linker + 3′motif |
| | | ATAAGGCTAGTCCGTTATCAACTT | replacement | |
| | | GAAAAAGTGGCACCGAGTCGGTGC | | |
| | | GCGGCGGCGGCGGCGGCGGTGGC | | |
| | | GGCTGTTGCTGCTGCTGGAAGGAC | | |
| | | TTGAGGGACTCGAAGGCTTTCATC | | |
| | | AGCTTTTCCAATCACTCGCGGTTCT | | |
| | | ATCTAGTTACGCGTTAAACCAACT | | |
| | | AGAA | | |
| htt_h31_ | 503 | GACCCTGGAAAAGCTGATGAGTTT | epegRNA for | spacer + scaffold + |
| p10eq1_5CAG | | TAGAGCTAGAAATAGCAAGTTAAA | various CAG | flap + linker + 3′motif |
| | | ATAAGGCTAGTCCGTTATCAACTT | replacement | |
| | | GAAAAAGTGGCACCGAGTCGGTGC | | |
| | | GCGGCGGCGGCGGCGGCGGTGGC | | |
| | | GGCTGTTGCTGCTGCTGCTGCTGG | | |
| | | AAGGACTTGAGGGACTCGAAGGCT | | |
| | | TTCATCAGCTTTTCCAATCACTCGC | | |
| | | GGTTCTATCTAGTTACGCGTTAAA | | |
| | | CCAACTAGAA | | |
| htt_h31_ | 504 | GACCCTGGAAAAGCTGATGAGTTT | epegRNA for | spacer + scaffold + |
| p10eq1_6CAG | | TAGAGCTAGAAATAGCAAGTTAAA | various CAG | flap + linker + 3′motif |
| | | ATAAGGCTAGTCCGTTATCAACTT | replacement | |
| | | GAAAAAGTGGCACCGAGTCGGTGC | | |
| | | GCGGCGGCGGCGGCGGCGGTGGC | | |
| | | GGCTGTTGCTGCTGCTGCTGCTGCT | | |
| | | GGAAGGACTTGAGGGACTCGAAG | | |
| | | GCTTTCATCAGCTTTTCCAATCACT | | |
| | | CGCGGTTCTATCTAGTTACGCGTTA | | |
| | | AACCAACTAGAA | | |
| htt_h31_ | 505 | GACCCTGGAAAAGCTGATGAGTTT | epegRNA for | spacer + scaffold + |
| p10eq1_6CAR | | TAGAGCTAGAAATAGCAAGTTAAA | various CAG | flap + linker + 3′motif |
| | | ATAAGGCTAGTCCGTTATCAACTT | replacement | |
| | | GAAAAAGTGGCACCGAGTCGGTGC | | |
| | | GCGGCGGCGGCGGCGGCGGTGGC | | |
| | | GGCTGTTGCTGCTGTTGCTGCTGCT | | |
| | | GGAAGGACTTGAGGGACTCGAAG | | |
| | | GCTTTCATCAGCTTTTCCAATCACT | | |
| | | CGCGGTTCTATCTAGTTACGCGTTA | | |
| | | AACCAACTAGAA | | |
| htt_h31_ | 506 | GACCCTGGAAAAGCTGATGAGTTT | epegRNA for | spacer + scaffold + |
| p10eq1_7CAG | | TAGAGCTAGAAATAGCAAGTTAAA | various CAG | flap + linker + 3′motif |
| | | ATAAGGCTAGTCCGTTATCAACTT | replacement | |
| | | GAAAAAGTGGCACCGAGTCGGTGC | | |
| | | GCGGCGGCGGCGGCGGCGGTGGC | | |
| | | GGCTGTTGCTGCTGCTGCTGCTGCT | | |
| | | GCTGGAAGGACTTGAGGGACTCGA | | |
| | | AGGCTTTCATCAGCTTTTCCAATCA | | |
| | | CTCGCGGTTCTATCTAGTTACGCGT | | |
| | | TAAACCAACTAGAA | | |
| htt_h31_ | 507 | GACCCTGGAAAAGCTGATGAGTTT | epegRNA for | spacer + scaffold + |
| p10eq1_8CAG | | TAGAGCTAGAAATAGCAAGTTAAA | various CAG | flap + linker + 3′motif |
| | | ATAAGGCTAGTCCGTTATCAACTT | replacement | |
| | | GAAAAAGTGGCACCGAGTCGGTGC | | |
| | | GCGGCGGCGGCGGCGGCGGTGGC | | |
| | | GGCTGTTGCTGCTGCTGCTGCTGCT | | |
| | | GCTGCTGGAAGGACTTGAGGGACT | | |
| | | CGAAGGCTTTCATCAGCTTTTCCA | | |
| | | ATCACTCGCGGTTCTATCTAGTTAC | | |
| | | GCGTTAAACCAACTAGAA | | |
| htt_h31_ | 508 | GACCCTGGAAAAGCTGATGAGTTT | epegRNA for | spacer + scaffold + |
| p10eq1_8CAR | | TAGAGCTAGAAATAGCAAGTTAAA | various CAG | flap + linker + 3′motif |
| | | ATAAGGCTAGTCCGTTATCAACTT | replacement | |
| | | GAAAAAGTGGCACCGAGTCGGTGC | | |
| | | GCGGCGGCGGCGGCGGCGGTGGC | | |
| | | GGCTGTTGCTGCTGCTGTTGCTGCT | | |
| | | GCTGCTGGAAGGACTTGAGGGACT | | |
| | | CGAAGGCTTTCATCAGCTTTTCCA | | |
| | | ATCACTCGCGGTTCTATCTAGTTAC | | |
| | | GCGTTAAACCAACTAGAA | | |
| htt_h31_ | 509 | GACCCTGGAAAAGCTGATGAGTTT | epegRNA for | spacer + scaffold + |
| p10eq1_9CAG | | TAGAGCTAGAAATAGCAAGTTAAA | various CAG | flap + linker + 3′motif |
| | | ATAAGGCTAGTCCGTTATCAACTT | replacement | |
| | | GAAAAAGTGGCACCGAGTCGGTGC | | |
| | | GCGGCGGCGGCGGCGGCGGTGGC | | |
| | | GGCTGTTGCTGCTGCTGCTGCTGCT | | |
| | | GCTGCTGCTGGAAGGACTTGAGGG | | |
| | | ACTCGAAGGCTTTCATCAGCTTTTC | | |
| | | CAATCACTCGCGGTTCTATCTAGTT | | |
| | | ACGCGTTAAACCAACTAGAA | | |
| htt_h40_ | 510 | GACCCTGGAAAAGCTGATGAGTTT | epegRNA for | spacer + scaffold + |
| p10eq1_2CAG | | TAGAGCTAGAAATAGCAAGTTAAA | various CAG | flap + linker + 3′motif |
| | | ATAAGGCTAGTCCGTTATCAACTT | replacement | |
| | | GAAAAAGTGGCACCGAGTCGGTGC | | |
| | | GAGGAGGCGGCGGCGGCGGCGGC | | |
| | | GGCGGTGGCGGCTGTTGCTGCTGG | | |
| | | AAGGACTTGAGGGACTCGAAGGCT | | |
| | | TTCATCAGCTTTTCCAATCACTCGC | | |
| | | GGTTCTATCTAGTTACGCGTTAAA | | |
| | | CCAACTAGAA | | |
| htt_h40_ | 511 | GACCCTGGAAAAGCTGATGAGTTT | epegRNA for | spacer + scaffold + |
| p10eq1_3CAG | | TAGAGCTAGAAATAGCAAGTTAAA | various CAG | flap + linker + 3′motif |
| | | ATAAGGCTAGTCCGTTATCAACTT | replacement | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGGAAGGACTTGAGGGACTCGAAG | | |
| | | GCTTTCATCAGCTTTTCCAATCACT | | |
| | | CGCGGTTCTATCTAGTTACGCGTTA | | |
| | | AACCAACTAGAA | | |
| htt_h40_ | 512 | GACCCTGGAAAAGCTGATGAGTTT | epegRNA for | spacer + scaffold + |
| p10eq1_5CAG | | TAGAGCTAGAAATAGCAAGTTAAA | various CAG | flap + linker + 3′motif |
| | | ATAAGGCTAGTCCGTTATCAACTT | replacement | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGCTGGAAGGACTTGAGGGAC | | |
| | | TCGAAGGCTTTCATCAGCTTTTCCA | | |
| | | ATCACTCGCGGTTCTATCTAGTTAC | | |
| | | GCGTTAAACCAACTAGAA | | |
| htt_h40_ | 513 | GACCCTGGAAAAGCTGATGAGTTT | epegRNA for | spacer + scaffold + |
| p10eq1_6CAG | | TAGAGCTAGAAATAGCAAGTTAAA | various CAG | flap + linker + 3′motif |
| | | ATAAGGCTAGTCCGTTATCAACTT | replacement | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGCTGCTGGAAGGACTTGAGG | | |
| | | GACTCGAAGGCTTTCATCAGCTTTT | | |
| | | CCAATCACTCGCGGTTCTATCTAGT | | |
| | | TACGCGTTAAACCAACTAGAA | | |
| htt_h40_ | 514 | GACCCTGGAAAAGCTGATGAGTTT | epegRNA for | spacer + scaffold + |
| p10eq1_7CAG | | TAGAGCTAGAAATAGCAAGTTAAA | various CAG | flap + linker + 3′motif |
| | | ATAAGGCTAGTCCGTTATCAACTT | replacement | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGCTGCTGCTGGAAGGACTTG | | |
| | | AGGGACTCGAAGGCTTTCATCAGC | | |
| | | TTTTCCAATCACTCGCGGTTCTATC | | |
| | | TAGTTACGCGTTAAACCAACTAGA | | |
| | | A | | |
| htt_h40_ | 515 | GACCCTGGAAAAGCTGATGAGTTT | epegRNA for | spacer + scaffold + |
| p10eq1_8CAG | | TAGAGCTAGAAATAGCAAGTTAAA | various CAG | flap + linker + 3′motif |
| | | ATAAGGCTAGTCCGTTATCAACTT | replacement | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGCTGCTGCTGCTGGAAGGAC | | |
| | | TTGAGGGACTCGAAGGCTTTCATC | | |
| | | AGCTTTTCCAATCACTCGCGGTTCT | | |
| | | ATCTAGTTACGCGTTAAACCAACT | | |
| | | AGAA | | |
| htt_h40_ | 516 | GACCCTGGAAAAGCTGATGAGTTT | epegRNA for | spacer + scaffold + |
| p10eq1_9CAG | | TAGAGCTAGAAATAGCAAGTTAAA | various CAG | flap + linker + 3′motif |
| | | ATAAGGCTAGTCCGTTATCAACTT | replacement | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGCTGCTGCTGCTGCTGGAAG | | |
| | | GACTTGAGGGACTCGAAGGCTTTC | | |
| | | ATCAGCTTTTCCAATCACTCGCGGT | | |
| | | TCTATCTAGTTACGCGTTAAACCA | | |
| | | ACTAGAA | | |
| Nick_5_4CAG | 395 | GCTGTTGCTGCTGCTGCTGC | nicking guide | spacer sequence |
| N6 | 383 | GCTGCTGCTGCTGCTGCTGGA | nicking guide | spacer sequence |
| N7 | 382 | GCTGCTGCTGGAAGGACTTG | nicking guide | spacer sequence |
| N14 | 396 | GGCCTTCATCAGCTTTTCC | nicking guide | spacer sequence |
| N14-3b | 397 | GGCTTTCATCAGCTTTTCC | nicking guide | spacer sequence |
| N15 | 398 | GGGACTCGAAGGCCTTCAT | nicking guide | spacer sequence |
| N16 | 399 | GGAAGGACTTGAGGGACTCG | nicking guide | spacer sequence |
| N17 | 400 | GCTGCTGCTGGAAGGACTT | nicking guide | spacer sequence |
| N18 | 401 | GCTGCTGCTGCTGCTGCTGG | nicking guide | spacer sequence |
| N19 | 402 | GCTGGAAGGACTTGAGGGACT | nicking guide | spacer sequence |
| N20 | 403 | GCTGCTGCTGCTGGAAGGACT | nicking guide | spacer sequence |
| N21 | 404 | CTGCTGCTGCTGCTGCTGGAA | nicking guide | spacer sequence |
| N22 | 405 | GCTGCTGCTGCTGCTGCTGCT | nicking guide | spacer sequence |
| N23 | 406 | GCTGCTGGAAGGACTTGAG | nicking guide | spacer sequence |
| N23b-GC2 | 407 | GCTGCTGGAAGGACTTCAG | nicking guide | spacer sequence |
| | | | specific to | |
| | | | 4CAG GC2 | |
| | | | edit | |
| N23b- | 408 | GCTGCTGAAAGGACTTCAG | nicking guide | spacer sequence |
| GC2GA1 | | | specific to | |
| | | | 4CAG edit and | |
| | | | GA1 mutation | |
| N23b- | 409 | GCTGTTGGAAGGACTTCAG | nicking guide | spacer sequence |
| GC29CAR5 | | | specific to | |
| | | | 9CAR5 | |
| htt_h31_ | 517 | GACCCTGGAAAAGCTGATGAGTTT | epegRNA with | spacer + scaffold + |
| pbs10UAeq1 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGGAAG | | |
| | | GACTTGAGGGACTCGAAGGCTTTC | | |
| | | ATCAGCTTTTCCAATCACTCGCGGT | | |
| | | TCTATCTAGTTACGCGTTAAACCA | | |
| | | ACTAGAA | | |
| htt_h35_ | 518 | GACCCTGGAAAAGCTGATGAGTTT | epegRNA with | spacer + scaffold + |
| pbs10UAeq1 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GGCGGCGGCGGCGGCGGCGGCGG | | |
| | | TGGCGGCTGTTGCTGCTGCTGCTG | | |
| | | GAAGGACTTGAGGGACTCGAAGG | | |
| | | CTTTCATCAGCTTTTCCAATCACTC | | |
| | | GCGGTTCTATCTAGTTACGCGTTA | | |
| | | AACCAACTAGAA | | |
| htt_h40_ | 519 | GACCCTGGAAAAGCTGATGAGTTT | epegRNA with | spacer + scaffold + |
| pbs10UAeq1 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGGAAGGACTTGAGGGACTCG | | |
| | | AAGGCTTTCATCAGCTTTTCCAATC | | |
| | | ACTCGCGGTTCTATCTAGTTACGC | | |
| | | GTTAAACCAACTAGAA | | |
| htt_h31_ | 520 | GACCCTGGAAAAGCTGATGAGTTT | epegRNA with | spacer + scaffold + |
| p10UAeq1_9CAG | | AAGAGCTAGAAATAGCAAGTTTAA | UA flip | flap + linker + 3′motif |
| | | ATAAGGCTAGTCCGTTATCAACTT | | |
| | | GAAAAAGTGGCACCGAGTCGGTGC | | |
| | | GCGGCGGCGGCGGCGGCGGTGGC | | |
| | | GGCTGTTGCTGCTGCTGCTGCTGCT | | |
| | | GCTGCTGCTGGAAGGACTTGAGGG | | |
| | | ACTCGAAGGCTTTCATCAGCTTTTC | | |
| | | CAATCACTCGCGGTTCTATCTAGTT | | |
| | | ACGCGTTAAACCAACTAGAA | | |
| htt_h40_ | 521 | GACCCTGGAAAAGCTGATGAGTTT | epegRNA with | spacer + scaffold + |
| p10UAeq1_9CAG | | AAGAGCTAGAAATAGCAAGTTTAA | UA flip | flap + linker + 3′motif |
| | | ATAAGGCTAGTCCGTTATCAACTT | | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGCTGCTGCTGCTGCTGGAAG | | |
| | | GACTTGAGGGACTCGAAGGCTTTC | | |
| | | ATCAGCTTTTCCAATCACTCGCGGT | | |
| | | TCTATCTAGTTACGCGTTAAACCA | | |
| | | ACTAGAA | | |
| htt_h31_ | 522 | GACCCTGGAAAAGCTGATGAGTTT | epegRNA for | spacer + scaffold + |
| pbs10UAeq1_ | | AAGAGCTAGAAATAGCAAGTTTAA | various CAG | flap + linker + 3′motif |
| 2CAR | | ATAAGGCTAGTCCGTTATCAACTT | replacement, | |
| | | GAAAAAGTGGCACCGAGTCGGTGC | with UA flip | |
| | | GCGGCGGCGGCGGCGGCGGTGGC | | |
| | | GGCTGTTGCTGTTGCTGCTGGAAG | | |
| | | GACTTGAGGGACTCGAAGGCTTTC | | |
| | | ATCAGCTTTTCCAATCACTCGCGGT | | |
| | | TCTATCTAGTTACGCGTTAAACCA | | |
| | | ACTAGAA | | |
| htt_h31_ | 523 | GACCCTGGAAAAGCTGATGAGTTT | epegRNA for | spacer + scaffold + |
| pbs10UAeq1_ | | AAGAGCTAGAAATAGCAAGTTTAA | various CAG | flap + linker + 3′motif |
| 4CAR | | ATAAGGCTAGTCCGTTATCAACTT | replacement, | |
| | | GAAAAAGTGGCACCGAGTCGGTGC | with UA flip | |
| | | GCGGCGGCGGCGGCGGCGGTGGC | | |
| | | GGCTGTTGCTGTTGCTGTTGGAAG | | |
| | | GACTTGAGGGACTCGAAGGCTTTC | | |
| | | ATCAGCTTTTCCAATCACTCGCGGT | | |
| | | TCTATCTAGTTACGCGTTAAACCA | | |
| | | ACTAGAA | | |
| htt_h31_ | 524 | GACCCTGGAAAAGCTGATGAGTTT | epegRNA for | spacer + scaffold + |
| pbs10UAeq1_ | | AAGAGCTAGAAATAGCAAGTTTAA | various CAG | flap + linker + 3′motif |
| CAA | | ATAAGGCTAGTCCGTTATCAACTT | replacement, | |
| | | GAAAAAGTGGCACCGAGTCGGTGC | 4CAA | |
| | | GCGGCGGCGGCGGCGGCGGTGGC | insertion (6Q), | |
| | | GGCTGTTGTTGTTGTTGTTGGAAG | with UA flip | |
| | | GACTTGAGGGACTCGAAGGCTTTC | | |
| | | ATCAGCTTTTCCAATCACTCGCGGT | | |
| | | TCTATCTAGTTACGCGTTAAACCA | | |
| | | ACTAGAA | | |
| htt_h31_ | 525 | GACCCTGGAAAAGCTGATGAGTTT | epegRNA for | spacer + scaffold + |
| pbs10UAeq1_ | | AAGAGCTAGAAATAGCAAGTTTAA | various CAG | flap + linker + 3′motif |
| 2CAR_GC2 | | ATAAGGCTAGTCCGTTATCAACTT | replacement, | |
| | | GAAAAAGTGGCACCGAGTCGGTGC | with UA flip | |
| | | GCGGCGGCGGCGGCGGCGGTGGC | | |
| | | GGCTGTTGCTGTTGCTGCTGGAAG | | |
| | | GACTTCAGGGACTCGAAGGCTTTC | | |
| | | ATCAGCTTTTCCAATCACTCGCGGT | | |
| | | TCTATCTAGTTACGCGTTAAACCA | | |
| | | ACTAGAA | | |
| htt_h31_ | 526 | GACCCTGGAAAAGCTGATGAGTTT | epegRNA for | spacer + scaffold + |
| pbs10UAeq1_ | | AAGAGCTAGAAATAGCAAGTTTAA | various CAG | flap + linker + 3′motif |
| 4CAR_GC2 | | ATAAGGCTAGTCCGTTATCAACTT | replacement, | |
| | | GAAAAAGTGGCACCGAGTCGGTGC | with UA flip | |
| | | GCGGCGGCGGCGGCGGCGGTGGC | | |
| | | GGCTGTTGCTGTTGCTGTTGGAAG | | |
| | | GACTTCAGGGACTCGAAGGCTTTC | | |
| | | ATCAGCTTTTCCAATCACTCGCGGT | | |
| | | TCTATCTAGTTACGCGTTAAACCA | | |
| | | ACTAGAA | | |
| htt_h31_ | 527 | GACCCTGGAAAAGCTGATGAGTTT | epegRNA for | spacer + scaffold + |
| pbs10UAeq1_ | | AAGAGCTAGAAATAGCAAGTTTAA | various CAG | flap + linker + 3′motif |
| CAA_GC2 | | ATAAGGCTAGTCCGTTATCAACTT | replacement, | |
| | | GAAAAAGTGGCACCGAGTCGGTGC | with UA flip | |
| | | GCGGCGGCGGCGGCGGCGGTGGC | | |
| | | GGCTGTTGTTGTTGTTGTTGGAAG | | |
| | | GACTTCAGGGACTCGAAGGCTTTC | | |
| | | ATCAGCTTTTCCAATCACTCGCGGT | | |
| | | TCTATCTAGTTACGCGTTAAACCA | | |
| | | ACTAGAA | | |
| htt_h31_ | 528 | GACCCTGGAAAAGCTGATGAGTTT | epegRNA for | spacer + scaffold + |
| pbs10UAeq1_ | | AAGAGCTAGAAATAGCAAGTTTAA | various CAG | flap + linker + 3′motif |
| CAArec | | ATAAGGCTAGTCCGTTATCAACTT | replacement, | |
| | | GAAAAAGTGGCACCGAGTCGGTGC | with UA flip | |
| | | GCGGCGGCGGCGGCGGCGGTGGC | | |
| | | GGCTGTTGTTGTTGTTGTTGAAACG | | |
| | | ATTTCAGCGATTCAAACGCTTTCAT | | |
| | | CAGCTTTTCCAATCACTCGCGGTTC | | |
| | | TATCTAGTTACGCGTTAAACCAAC | | |
| | | TAGAA | | |
| htt_h31_ | 529 | GACCCTGGAAAAGCTGATGAGTTT | epegRNA with | spacer + scaffold + |
| pbs10UAeq1_ | | AAGAGCTAGAAATAGCAAGTTTAA | UA flip, 4CAG | flap + linker + 3′motif |
| GC2 | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGGAAG | | |
| | | GACTTCAGGGACTCGAAGGCTTTC | | |
| | | ATCAGCTTTTCCAATCACTCGCGGT | | |
| | | TCTATCTAGTTACGCGTTAAACCA | | |
| | | ACTAGAA | | |
| htt_h31_ | 530 | GACCCTGGAAAAGCTGATGAGTTT | epegRNA with | spacer + scaffold + |
| pbs10UAeq1_ | | AAGAGCTAGAAATAGCAAGTTTAA | UA flip, 4CAG | flap + linker + 3′motif |
| GC2CT2 | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGGAAG | | |
| | | GACTTCAGGGATTCGAAGGCTTTC | | |
| | | ATCAGCTTTTCCAATCACTCGCGGT | | |
| | | TCTATCTAGTTACGCGTTAAACCA | | |
| | | ACTAGAA | | |
| htt_h40_ | 531 | GACCCTGGAAAAGCTGATGAGTTT | epegRNA with | spacer + scaffold + |
| pbs10UAeq1_ | | AAGAGCTAGAAATAGCAAGTTTAA | UA flip, 4CAG | flap + linker + 3′motif |
| GC2 | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGGAAGGACTTCAGGGACTCG | | |
| | | AAGGCTTTCATCAGCTTTTCCAATC | | |
| | | ACTCGCGGTTCTATCTAGTTACGC | | |
| | | GTTAAACCAACTAGAA | | |
| htt_h31_ | 532 | GACCCTGGAAAAGCTGATGAGTTT | epegRNA with | spacer + scaffold + |
| pbs10UAeq1_ | | AAGAGCTAGAAATAGCAAGTTTAA | UA flip, 4CAG | flap + linker + 3′motif |
| GC2CT2GA1 | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGAAAG | | |
| | | GACTTCAGGGATTCGAAGGCTTTC | | |
| | | ATCAGCTTTTCCAATCACTCGCGGT | | |
| | | TCTATCTAGTTACGCGTTAAACCA | | |
| | | ACTAGAA | | |
| htt_h31_ | 530 | GACCCTGGAAAAGCTGATGAGTTT | epegRNA with | spacer + scaffold + |
| pbs10UAeq1_ | | AAGAGCTAGAAATAGCAAGTTTAA | UA flip, 4CAG | flap + linker + 3′motif |
| GC2CT2GA2 | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGGAAG | | |
| | | GACTTCAGGGATTCGAAGGCTTTC | | |
| | | ATCAGCTTTTCCAATCACTCGCGGT | | |
| | | TCTATCTAGTTACGCGTTAAACCA | | |
| | | ACTAGAA | | |
| htt_h31_ | 533 | GACCCTGGAAAAGCTGATGAGTTT | epegRNA for | spacer + scaffold + |
| pbs10UAeq1_ | | AAGAGCTAGAAATAGCAAGTTTAA | various CAG | flap + linker + 3′motif |
| 9CAG_GC2CT2 | | ATAAGGCTAGTCCGTTATCAACTT | replacement | |
| | | GAAAAAGTGGCACCGAGTCGGTGC | with UA flip | |
| | | GCGGCGGCGGCGGCGGCGGTGGC | | |
| | | GGCTGTTGCTGCTGCTGCTGCTGCT | | |
| | | GCTGCTGCTGGAAGGACTTCAGGG | | |
| | | ATTCGAAGGCTTTCATCAGCTTTTC | | |
| | | CAATCACTCGCGGTTCTATCTAGTT | | |
| | | ACGCGTTAAACCAACTAGAA | | |
| htt_h31_ | 534 | GACCCTGGAAAAGCTGATGAGTTT | epegRNA for | spacer + scaffold + |
| pbs10UAeq1_ | | AAGAGCTAGAAATAGCAAGTTTAA | various CAG | flap + linker + 3′motif |
| 9CAR5 | | ATAAGGCTAGTCCGTTATCAACTT | replacement | |
| | | GAAAAAGTGGCACCGAGTCGGTGC | with UA flip | |
| | | GCGGCGGCGGCGGCGGCGGTGGC | | |
| | | GGCTGTTGTTGCTGTTGCTGTTGCT | | |
| | | GTTGCTGTTGGAAGGACTTGAGGG | | |
| | | ACTCGAAGGCTTTCATCAGCTTTTC | | |
| | | CAATCACTCGCGGTTCTATCTAGTT | | |
| | | ACGCGTTAAACCAACTAGAA | | |
| htt_h31_ | 535 | GACCCTGGAAAAGCTGATGAGTTT | epegRNA for | spacer + scaffold + |
| pbs10UAeq1_ | | AAGAGCTAGAAATAGCAAGTTTAA | various CAG | flap + linker + 3′motif |
| 9CAR5_GC2CT2 | | ATAAGGCTAGTCCGTTATCAACTT | replacement | |
| | | GAAAAAGTGGCACCGAGTCGGTGC | with UA flip | |
| | | GCGGCGGCGGCGGCGGCGGTGGC | | |
| | | GGCTGTTGTTGCTGTTGCTGTTGCT | | |
| | | GTTGCTGTTGGAAGGACTTCAGGG | | |
| | | ATTCGAAGGCTTTCATCAGCTTTTC | | |
| | | CAATCACTCGCGGTTCTATCTAGTT | | |
| | | ACGCGTTAAACCAACTAGAA | | |
| htt_h40_ | 536 | GACCCTGGAAAAGCTGATGAGTTT | epegRNA for | spacer + scaffold + |
| pbs10UAeq1_ | | AAGAGCTAGAAATAGCAAGTTTAA | various CAG | flap + linker + 3′motif |
| 9CAG_GC2CT2 | | ATAAGGCTAGTCCGTTATCAACTT | replacement | |
| | | GAAAAAGTGGCACCGAGTCGGTGC | with UA flip | |
| | | GAGGAGGCGGCGGCGGCGGCGGC | | |
| | | GGCGGTGGCGGCTGTTGCTGCTGC | | |
| | | TGCTGCTGCTGCTGCTGCTGGAAG | | |
| | | GACTTCAGGGATTCGAAGGCTTTC | | |
| | | ATCAGCTTTTCCAATCACTCGCGGT | | |
| | | TCTATCTAGTTACGCGTTAAACCA | | |
| | | ACTAGAA | | |
| htt_h40_ | 537 | GACCCTGGAAAAGCTGATGAGTTT | epegRNA with | spacer + scaffold + |
| pbs10UAeq1_ | | AAGAGCTAGAAATAGCAAGTTTAA | UA flip, 4CAG | flap + linker + 3′motif |
| 9CAR5 | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGTTGCTGT | | |
| | | TGCTGTTGCTGTTGCTGTTGGAAG | | |
| | | GACTTGAGGGACTCGAAGGCTTTC | | |
| | | ATCAGCTTTTCCAATCACTCGCGGT | | |
| | | TCTATCTAGTTACGCGTTAAACCA | | |
| | | ACTAGAA | | |
| htt_h40_ | 538 | GACCCTGGAAAAGCTGATGAGTTT | epegRNA with | spacer + scaffold + |
| pbs10UAeq1_ | | AAGAGCTAGAAATAGCAAGTTTAA | UA flip, 4CAG | flap + linker + 3′motif |
| 9CAR5_GC2CT2 | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGTTGCTGT | | |
| | | TGCTGTTGCTGTTGCTGTTGGAAG | | |
| | | GACTTCAGGGATTCGAAGGCTTTC | | |
| | | ATCAGCTTTTCCAATCACTCGCGGT | | |
| | | TCTATCTAGTTACGCGTTAAACCA | | |
| | | ACTAGAA | | |
| htt_h31_G2toC | 539 | GACCCTGGAAAAGCTGATGAGTTT | epegRNA with | spacer + scaffold + |
| | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGGAAG | | |
| | | GACTTGAGGGACTCGAAGGCGTTC | | |
| | | ATCAGCTTTTCCAATCACTCGCGGT | | |
| | | TCTATCTAGTTACGCGTTAAACCA | | |
| | | ACTAGAA | | |
| htt_h31_G3toC | 540 | GACCCTGGAAAAGCTGATGAGTTT | epegRNA with | spacer + scaffold + |
| | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGGAAG | | |
| | | GACTTGAGGGACTCGAAGGGCTTC | | |
| | | ATCAGCTTTTCCAATCACTCGCGGT | | |
| | | TCTATCTAGTTACGCGTTAAACCA | | |
| | | ACTAGAA | | |
| htt_h31_2GtoC | 541 | GACCCTGGAAAAGCTGATGAGTTT | epegRNA with | spacer + scaffold + |
| | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGGAAG | | |
| | | GACTTGAGGGACTCGAAGGGGTTC | | |
| | | ATCAGCTTTTCCAATCACTCGCGGT | | |
| | | TCTATCTAGTTACGCGTTAAACCA | | |
| | | ACTAGAA | | |
| htt_h31_3GC | 542 | GACCCTGGAAAAGCTGATGAGTTT | epegRNA with | spacer + scaffold + |
| | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGGAAC | | |
| | | GACTTCAGCGACTCGAAGGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GC1 | 543 | GACCCTGGAAAAGCTGATGAGTTT | epegRNA with | spacer + scaffold + |
| | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGGAAC | | |
| | | GACTTGAGGGACTCGAAGGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GC3 | 544 | GACCCTGGAAAAGCTGATGAGTTT | epegRNA with | spacer + scaffold + |
| | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGGAAG | | |
| | | GACTTGAGCGACTCGAAGGCTTTC | | |
| | | ATCAGCTTTTCCAATCACTCGCGGT | | |
| | | TCTATCTAGTTACGCGTTAAACCA | | |
| | | ACTAGAA | | |
| htt_h31_CT1 | 545 | GACCCTGGAAAAGCTGATGAGTTT | epegRNA with | spacer + scaffold + |
| | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGGAAG | | |
| | | GATTTGAGGGACTCGAAGGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GA1 | 546 | GACCCTGGAAAAGCTGATGAGTTT | epegRNA with | spacer + scaffold + |
| | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGAAAG | | |
| | | GACTTGAGGGACTCGAAGGCTTTC | | |
| | | ATCAGCTTTTCCAATCACTCGCGGT | | |
| | | TCTATCTAGTTACGCGTTAAACCA | | |
| | | ACTAGAA | | |
| htt_h31_CT2 | 547 | GACCCTGGAAAAGCTGATGAGTTT | epegRNA with | spacer + scaffold + |
| | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGGAAG | | |
| | | GACTTGAGGGATTCGAAGGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GA2 | 548 | GACCCTGGAAAAGCTGATGAGTTT | epegRNA with | spacer + scaffold + |
| | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGGAAG | | |
| | | GACTTGAGGGACTCAAAGGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GC4 | 549 | GACCCTGGAAAAGCTGATGAGTTT | epegRNA with | spacer + scaffold + |
| | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGGAAG | | |
| | | GACTTGAGGGACTCGAACGCTTTC | | |
| | | ATCAGCTTTTCCAATCACTCGCGGT | | |
| | | TCTATCTAGTTACGCGTTAAACCA | | |
| | | ACTAGAA | | |
| htt_h31_ | 550 | GACCCTGGAAAAGCTGATGAGTTT | epegRNA with | spacer + scaffold + |
| CT1_GC2 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGGAAG | | |
| | | GATTTCAGGGACTCGAAGGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GA1_ | 551 | GACCCTGGAAAAGCTGATGAGTTT | epegRNA with | spacer + scaffold + |
| GC2 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGAAAG | | |
| | | GACTTCAGGGACTCGAAGGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GA2_ | 552 | GACCCTGGAAAAGCTGATGAGTTT | epegRNA with | spacer + scaffold + |
| GC2 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGGAAG | | |
| | | GACTTCAGGGACTCAAAGGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GC4_ | 553 | GACCCTGGAAAAGCTGATGAGTTT | epegRNA with | spacer + scaffold + |
| GC2 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGGAAG | | |
| | | GACTTCAGGGACTCGAACGCTTTC | | |
| | | ATCAGCTTTTCCAATCACTCGCGGT | | |
| | | TCTATCTAGTTACGCGTTAAACCA | | |
| | | ACTAGAA | | |
| htt_ | 554 | GACCCTGGAAAAGCTGATGAGTTT | epegRNA with | spacer + scaffold + |
| h31p10seed_n16eq | | AAGAGCTAGAAATAGCAAGTTTAA | UA flip, 4CAG | flap + linker + 3′motif |
| (GC2 + 3) | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGGAAG | | |
| | | GACTTCAGCGACTCGAAGGCTTTC | | |
| | | ATCAGCTTTTCCAATCACTCGCGGT | | |
| | | TCTATCTAGTTACGCGTTAAACCA | | |
| | | ACTAGAA | | |
| htt_ | 555 | GACCCTGGAAAAGCTGATGAGTTT | epegRNA with | spacer + scaffold + |
| h31p10seed_n7eq | | AAGAGCTAGAAATAGCAAGTTTAA | UA flip, 4CAG | flap + linker + 3′motif |
| (GC1 + 2) | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGGAAC | | |
| | | GACTTCAGGGACTCGAAGGCTTTC | | |
| | | ATCAGCTTTTCCAATCACTCGCGGT | | |
| | | TCTATCTAGTTACGCGTTAAACCA | | |
| | | ACTAGAA | | |
| htt_h31_CT1_ | 556 | GACCCTGGAAAAGCTGATGAGTTT | epegRNA with | spacer + scaffold + |
| GA1 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGAAAG | | |
| | | GATTTGAGGGACTCGAAGGCTTTC | | |
| | | ATCAGCTTTTCCAATCACTCGCGGT | | |
| | | TCTATCTAGTTACGCGTTAAACCA | | |
| | | ACTAGAA | | |
| htt_h31_CT1_ | 557 | GACCCTGGAAAAGCTGATGAGTTT | epegRNA with | spacer + scaffold + |
| CT2 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGGAAG | | |
| | | GATTTGAGGGATTCGAAGGCTTTC | | |
| | | ATCAGCTTTTCCAATCACTCGCGGT | | |
| | | TCTATCTAGTTACGCGTTAAACCA | | |
| | | ACTAGAA | | |
| htt_h31_CT1_ | 558 | GACCCTGGAAAAGCTGATGAGTTT | epegRNA with | spacer + scaffold + |
| GA2 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGGAAG | | |
| | | GATTTGAGGGACTCAAAGGCTTTC | | |
| | | ATCAGCTTTTCCAATCACTCGCGGT | | |
| | | TCTATCTAGTTACGCGTTAAACCA | | |
| | | ACTAGAA | | |
| htt_h31_CT1_ | 559 | GACCCTGGAAAAGCTGATGAGTTT | epegRNA with | spacer + scaffold + |
| GC4 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGGAAG | | |
| | | GATTTGAGGGACTCGAACGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GA1_ | 560 | GACCCTGGAAAAGCTGATGAGTTT | epegRNA with | spacer + scaffold + |
| CT2 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGAAAG | | |
| | | GACTTGAGGGATTCGAAGGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GA1_ | 561 | GACCCTGGAAAAGCTGATGAGTTT | epegRNA with | spacer + scaffold + |
| GA2 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGAAAG | | |
| | | GACTTGAGGGACTCAAAGGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GA1_ | 562 | GACCCTGGAAAAGCTGATGAGTTT | epegRNA with | spacer + scaffold + |
| GC4 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGAAAG | | |
| | | GACTTGAGGGACTCGAACGCTTTC | | |
| | | ATCAGCTTTTCCAATCACTCGCGGT | | |
| | | TCTATCTAGTTACGCGTTAAACCA | | |
| | | ACTAGAA | | |
| htt_h31_CT2_ | 563 | GACCCTGGAAAAGCTGATGAGTTT | epegRNA with | spacer + scaffold + |
| GA2 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGGAAG | | |
| | | GACTTGAGGGATTCAAAGGCTTTC | | |
| | | ATCAGCTTTTCCAATCACTCGCGGT | | |
| | | TCTATCTAGTTACGCGTTAAACCA | | |
| | | ACTAGAA | | |
| htt_h31_CT2_ | 564 | GACCCTGGAAAAGCTGATGAGTTT | epegRNA with | spacer + scaffold + |
| GC4 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGGAAG | | |
| | | GACTTGAGGGATTCGAACGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GA2_ | 565 | GACCCTGGAAAAGCTGATGAGTTT | epegRNA with | spacer + scaffold + |
| GC4 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGGAAG | | |
| | | GACTTGAGGGACTCAAACGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GC3_ | 566 | GACCCTGGAAAAGCTGATGAGTTT | epegRNA with | spacer + scaffold + |
| CT1 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGGAAG | | |
| | | GATTTGAGCGACTCGAAGGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GC3_ | 567 | GACCCTGGAAAAGCTGATGAGTTT | epegRNA with | spacer + scaffold + |
| GA1 | | AAGAGCTAGAAATAGCAAGTTTAA | \|UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGAAAG | | |
| | | GACTTGAGCGACTCGAAGGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GC3_ | 568 | GACCCTGGAAAAGCTGATGAGTTT | epegRNA with | spacer + scaffold + |
| CT2 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGGAAG | | |
| | | GACTTGAGCGATTCGAAGGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GC3_ | 569 | GACCCTGGAAAAGCTGATGAGTTT | epegRNA with | spacer + scaffold + |
| GA2 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGGAAG | | |
| | | GACTTGAGCGACTCAAAGGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GC3_ | 570 | GACCCTGGAAAAGCTGATGAGTTT | epegRNA with | spacer + scaffold + |
| GC4 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGGAAG | | |
| | | GACTTGAGCGACTCGAACGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GC2_ | 571 | GACCCTGGAAAAGCTGATGAGTTT | epegRNA with | spacer + scaffold + |
| GC3_CT2 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGGAAG | | |
| | | GACTTCAGCGATTCGAAGGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GC2_ | 572 | GACCCTGGAAAAGCTGATGAGTTT | epegRNA with | spacer + scaffold + |
| GC3_GC4 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGGAAG | | |
| | | GACTTCAGCGACTCGAACGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GC2_ | 573 | GACCCTGGAAAAGCTGATGAGTTT | epegRNA with | spacer + scaffold + |
| GC3_CT1 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGGAAG | | |
| | | GATTTCAGCGACTCGAAGGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GC2_ | 574 | GACCCTGGAAAAGCTGATGAGTTT | epegRNA with | spacer + scaffold + |
| GC3_GA1 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGAAAG | | |
| | | GACTTCAGCGACTCGAAGGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GC2_ | 575 | GACCCTGGAAAAGCTGATGAGTTT | epegRNA with | spacer + scaffold + |
| GC3_GA2 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGGAAG | | |
| | | GACTTCAGCGACTCAAAGGCTTTC | | |
| | | ATCAGCTTTTCCAATCACTCGCGGT | | |
| | | TCTATCTAGTTACGCGTTAAACCA | | |
| | | ACTAGAA | | |
| htt_h31_CT1_ | 576 | GACCCTGGAAAAGCTGATGAGTTT | epegRNA with | spacer + scaffold + |
| GA1_CT2 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGAAAG | | |
| | | GATTTGAGGGATTCGAAGGCTTTC | | |
| | | ATCAGCTTTTCCAATCACTCGCGGT | | |
| | | TCTATCTAGTTACGCGTTAAACCA | | |
| | | ACTAGAA | | |
| htt_h31_CT1_ | 577 | GACCCTGGAAAAGCTGATGAGTTT | epegRNA with | spacer + scaffold + |
| GA1_GA2 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGAAAG | | |
| | | GATTTGAGGGACTCAAAGGCTTTC | | |
| | | ATCAGCTTTTCCAATCACTCGCGGT | | |
| | | TCTATCTAGTTACGCGTTAAACCA | | |
| | | ACTAGAA | | |
| htt_h31_CT1_ | 578 | GACCCTGGAAAAGCTGATGAGTTT | epegRNA with | spacer + scaffold + |
| GA1_GC4 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGAAAG | | |
| | | GATTTGAGGGACTCGAAcGCTTTC | | |
| | | ATCAGCTTTTCCAATCACTCGCGGT | | |
| | | TCTATCTAGTTACGCGTTAAACCA | | |
| | | ACTAGAA | | |
| htt_h31_CT1_ | 579 | GACCCTGGAAAAGCTGATGAGTTT | epegRNA with | spacer + scaffold + |
| GA1_GC2 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGAAAG | | |
| | | GATTTCAGGGACTCGAAGGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GA1_ | 580 | GACCCTGGAAAAGCTGATGAGTTT | epegRNA with | spacer + scaffold + |
| CT2_GA2 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGAAAG | | |
| | | GACTTGAGGGATTCAAAGGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GA1_ | 581 | GACCCTGGAAAAGCTGATGAGTTT | epegRNA with | spacer + scaffold + |
| CT2_GC4 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGAAAG | | |
| | | GACTTGAGGGATTCGAACGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GA1_ | 532 | GACCCTGGAAAAGCTGATGAGTTT | epegRNA with | spacer + scaffold + |
| CT2_GC2 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGAAAG | | |
| | | GACTTCAGGGATTCGAAGGCTTTC | | |
| | | ATCAGCTTTTCCAATCACTCGCGGT | | |
| | | TCTATCTAGTTACGCGTTAAACCA | | |
| | | ACTAGAA | | |
| htt_h31_CT2_ | 564 | GACCCTGGAAAAGCTGATGAGTTT | epegRNA with | spacer + scaffold + |
| GA2_GC4 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGGAAG | | |
| | | GACTTGAGGGATTCGAACGCTTTC | | |
| | | ATCAGCTTTTCCAATCACTCGCGGT | | |
| | | TCTATCTAGTTACGCGTTAAACCA | | |
| | | ACTAGAA | | |
| htt_h31_CT2_ | 530 | GACCCTGGAAAAGCTGATGAGTTT | epegRNA with | spacer + scaffold + |
| GA2_GC2 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGGAAG | | |
| | | GACTTCAGGGATTCGAAGGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GA2_ | 582 | GACCCTGGAAAAGCTGATGAGTTT | epegRNA with | spacer + scaffold + |
| GC4_GC2 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGGAAG | | |
| | | GACTTCAGGGACTCAAACGCTTTC | | |
| | | ATCAGCTTTTCCAATCACTCGCGGT | | |
| | | TCTATCTAGTTACGCGTTAAACCA | | |
| | | ACTAGAA | | |
| htt_h31_CT1 | 576 | GACCCTGGAAAAGCTGATGAGTTT | epegRNA with | spacer + scaffold + |
| CT2_GA2 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGAAAG | | |
| | | GATTTGAGGGATTCGAAGGCTTTC | | |
| | | ATCAGCTTTTCCAATCACTCGCGGT | | |
| | | TCTATCTAGTTACGCGTTAAACCA | | |
| | | ACTAGAA | | |
| htt_h31_CT1_ | 583 | GACCCTGGAAAAGCTGATGAGTTT | epegRNA with | spacer + scaffold + |
| CT2_GC4 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGGAAG | | |
| | | GATTTGAGGGATTCGAACGCTTTC | | |
| | | ATCAGCTTTTCCAATCACTCGCGGT | | |
| | | TCTATCTAGTTACGCGTTAAACCA | | |
| | | ACTAGAA | | |
| htt_h31_CT1_ | 584 | GACCCTGGAAAAGCTGATGAGTTT | epegRNA with | spacer + scaffold + |
| CT2_GC2 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGGAAG | | |
| | | GATTTCAGGGATTCGAAGGCTTTC | | |
| | | ATCAGCTTTTCCAATCACTCGCGGT | | |
| | | TCTATCTAGTTACGCGTTAAACCA | | |
| | | ACTAGAA | | |
| htt_h31_CT1_ | 585 | GACCCTGGAAAAGCTGATGAGTTT | epegRNA with | spacer + scaffold + |
| GA2_GC4 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGGAAG | | |
| | | GATTTGAGGGACTCAAACGCTTTC | | |
| | | ATCAGCTTTTCCAATCACTCGCGGT | | |
| | | TCTATCTAGTTACGCGTTAAACCA | | |
| | | ACTAGAA | | |
| htt_h31_CT1_ | 579 | GACCCTGGAAAAGCTGATGAGTTT | epegRNA with | spacer + scaffold + |
| GA2_GC2 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGAAAG | | |
| | | GATTTCAGGGACTCGAAGGCTTTC | | |
| | | ATCAGCTTTTCCAATCACTCGCGGT | | |
| | | TCTATCTAGTTACGCGTTAAACCA | | |
| | | ACTAGAA | | |
| htt_h31_CT1_ | 586 | GACCCTGGAAAAGCTGATGAGTTT | epegRNA with | spacer + scaffold + |
| GC3_CT2 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGGAAG | | |
| | | GATTTGAGCGATTCGAAGGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GA1_ | 587 | GACCCTGGAAAAGCTGATGAGTTT | epegRNA with | spacer + scaffold + |
| GC3_CT2 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGAAAG | | |
| | | GACTTGAGCGATTCGAAGGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GA2_ | 588 | GACCCTGGAAAAGCTGATGAGTTT | epegRNA with | spacer + scaffold + |
| GC3_CT2 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGGAAG | | |
| | | GACTTGAGCGATTCAAAGGCTTTC | | |
| | | ATCAGCTTTTCCAATCACTCGCGGT | | |
| | | TCTATCTAGTTACGCGTTAAACCA | | |
| | | ACTAGAA | | |
| htt_h31_CT1_ | 589 | GACCCTGGAAAAGCTGATGAGTTT | epegRNA with | spacer + scaffold + |
| GC3_GC4 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGGAAG | | |
| | | GATTTGAGCGACTCGAACGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GA1_ | 590 | GACCCTGGAAAAGCTGATGAGTTT | epegRNA with | spacer + scaffold + |
| GC3_GC4 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGAAAG | | |
| | | GACTTGAGCGACTCGAACGCTTTC | | |
| | | ATCAGCTTTTCCAATCACTCGCGGT | | |
| | | TCTATCTAGTTACGCGTTAAACCA | | |
| | | ACTAGAA | | |
| htt_h31_CT2_ | 591 | GACCCTGGAAAAGCTGATGAGTTT | epegRNA with | spacer + scaffold + |
| GC3_GC4 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGGAAG | | |
| | | GACTTGAGCGATTCGAACGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GA2_ | 592 | GACCCTGGAAAAGCTGATGAGTTT | epegRNA with | spacer + scaffold + |
| GC3_GC4 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGGAAG | | |
| | | GACTTGAGCGACTCAAACGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GA1_ | 593 | GACCCTGGAAAAGCTGATGAGTTT | epegRNA with | spacer + scaffold + |
| GC3_CT1 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGAAAG | | |
| | | GATTTGAGCGACTCGAAGGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GA2_ | 594 | GACCCTGGAAAAGCTGATGAGTTT | epegRNA with | spacer + scaffold + |
| GC3_CT1 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGGAAG | | |
| | | GATTTGAGCGACTCAAAGGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GC2_ | 595 | GACCCTGGAAAAGCTGATGAGTTT | epegRNA with | spacer + scaffold + |
| GC4_CT1 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGGAAG | | |
| | | GATTTCAGGGACTCGAACGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GC2_ | 596 | GACCCTGGAAAAGCTGATGAGTTT | epegRNA with | spacer + scaffold + |
| GC4_GA1 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGAAAG | | |
| | | GACTTCAGGGACTCGAACGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GC2_ | 597 | GACCCTGGAAAAGCTGATGAGTTT | epegRNA with | spacer + scaffold + |
| GC4_CT2 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGGAAG | | |
| | | GACTTCAGGGATTCGAACGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GC2_ | 598 | GACCCTGGAAAAGCTGATGAGTTT | epegRNA with | spacer + scaffold + |
| GA1_GA2 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGAAAG | | |
| | | GACTTCAGGGACTCAAAGGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GC3_ | 599 | GACCCTGGAAAAGCTGATGAGTTT | epegRNA with | spacer + scaffold + |
| GA1_GA2 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGAAAG | | |
| | | GACTTGAGCGACTCAAAGGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GC4_ | 600 | GACCCTGGAAAAGCTGATGAGTTT | epegRNA with | spacer + scaffold + |
| GA1_GA2 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGAAAG | | |
| | | GACTTGAGGGACTCAAACGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GC2_ | 601 | GACCCTGGAAAAGCTGATGAGTTT | epegRNA with | spacer + scaffold + |
| GC3_GC4_ | | AAGAGCTAGAAATAGCAAGTTTAA | UA flip, 4CAG | flap + linker + 3′motif |
| CT2 | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGGAAG | | |
| | | GACTTCAGCGATTCGAACGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GC2_ | 602 | GACCCTGGAAAAGCTGATGAGTTT | epegRNA with | spacer + scaffold + |
| GC3_GC4_ | | AAGAGCTAGAAATAGCAAGTTTAA | UA flip, 4CAG | flap + linker + 3′motif |
| CT1 | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGGAAG | | |
| | | GATTTCAGCGACTCGAACGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GC2_ | 603 | GACCCTGGAAAAGCTGATGAGTTT | epegRNA with | spacer + scaffold + |
| GC3_GC4_ | | AAGAGCTAGAAATAGCAAGTTTAA | UA flip, 4CAG | flap + linker + 3′motif |
| GA1 | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGAAAG | | |
| | | GACTTCAGCGACTCGAACGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GC2_ | 604 | GACCCTGGAAAAGCTGATGAGTTT | epegRNA with | spacer + scaffold + |
| GC3_GC4_ | | AAGAGCTAGAAATAGCAAGTTTAA | UA flip, 4CAG | flap + linker + 3′motif |
| GA2 | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGGAAG | | |
| | | GACTTCAGCGACTCAAACGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GC3_ | 605 | GACCCTGGAAAAGCTGATGAGTTT | epegRNA with | spacer + scaffold + |
| GC4_CT1_ | | AAGAGCTAGAAATAGCAAGTTTAA | UA flip, 4CAG | flap + linker + 3′motif |
| GA1 | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGAAAG | | |
| | | GATTTGAGCGACTCGAACGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GC3_ | 606 | GACCCTGGAAAAGCTGATGAGTTT | epegRNA with | spacer + scaffold + |
| GC4_CT1_ | | AAGAGCTAGAAATAGCAAGTTTAA | UA flip, 4CAG | flap + linker + 3′motif |
| CT2 | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGGAAG | | |
| | | GATTTGAGCGATTCGAACGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GC3 | 605 | GACCCTGGAAAAGCTGATGAGTTT | epegRNA with | spacer + scaffold + |
| GC4_CT1 | | AAGAGCTAGAAATAGCAAGTTTAA | UA flip, 4CAG | flap + linker + 3′motif |
| GA2 | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGAAAG | | |
| | | GATTTGAGCGACTCGAACGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GC4_ | 607 | GACCCTGGAAAAGCTGATGAGTTT | epegRNA with | spacer + scaffold + |
| CT1_GA1_ | | AAGAGCTAGAAATAGCAAGTTTAA | UA flip, 4CAG | flap + linker + 3′motif |
| CT2 | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGAAAG | | |
| | | GATTTGAGGGATTCGAAcGCTTTCA | | |
| | | TCAGCTTTTCCAATCACTCGCGGTT | | |
| | | CTATCTAGTTACGCGTTAAACCAA | | |
| | | CTAGAA | | |
| htt_h31_GC4_ | 608 | GACCCTGGAAAAGCTGATGAGTTT | epegRNA with | spacer + scaffold + |
| CT1_GA1_ | | AAGAGCTAGAAATAGCAAGTTTAA | UA flip, 4CAG | flap + linker + 3′motif |
| GA2 | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGAAAG | | |
| | | GATTTGAGGGACTCAAAcGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GC4_ | 609 | GACCCTGGAAAAGCTGATGAGTTT | epegRNA with | spacer + scaffold + |
| CT1_GA1_ | | AAGAGCTAGAAATAGCAAGTTTAA | UA flip, 4CAG | flap + linker + 3′motif |
| GC2 | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGAAAG | | |
| | | GATTTCAGGGACTCGAAcGCTTTCA | | |
| | | TCAGCTTTTCCAATCACTCGCGGTT | | |
| | | CTATCTAGTTACGCGTTAAACCAA | | |
| | | CTAGAA | | |
| htt_h31_CT1_ | 610 | GACCCTGGAAAAGCTGATGAGTTT | epegRNA with | spacer + scaffold + |
| GA1_CT2_ | | AAGAGCTAGAAATAGCAAGTTTAA | UA flip, 4CAG | flap + linker + 3′motif |
| GA2 | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGAAAG | | |
| | | GATTTGAGGGATTCAAAGGCTTTC | | |
| | | ATCAGCTTTTCCAATCACTCGCGGT | | |
| | | TCTATCTAGTTACGCGTTAAACCA | | |
| | | ACTAGAA | | |
| htt_h31_CT1_ | 611 | GACCCTGGAAAAGCTGATGAGTTT | epegRNA with | spacer + scaffold + |
| GA1_CT2_ | | AAGAGCTAGAAATAGCAAGTTTAA | UA flip, 4CAG | flap + linker + 3′motif |
| GC2 | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGAAAG | | |
| | | GATTTCAGGGATTCGAAGGCTTTC | | |
| | | ATCAGCTTTTCCAATCACTCGCGGT | | |
| | | TCTATCTAGTTACGCGTTAAACCA | | |
| | | ACTAGAA | | |
| htt_h31_CT1_ | 612 | GACCCTGGAAAAGCTGATGAGTTT | epegRNA with | spacer + scaffold + |
| GA1_CT2_ | | AAGAGCTAGAAATAGCAAGTTTAA | UA flip, 4CAG | flap + linker + 3′motif |
| GC3 | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGAAAG | | |
| | | GATTTGAGCGATTCGAAGGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GA1_ | 613 | GACCCTGGAAAAGCTGATGAGTTT | epegRNA with | spacer + scaffold + |
| CT2_GA2_ | | AAGAGCTAGAAATAGCAAGTTTAA | UA flip, 4CAG | flap + linker + 3′motif |
| GC2 | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGAAAG | | |
| | | GACTTCAGGGATTCAAAGGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GA1_ | 614 | GACCCTGGAAAAGCTGATGAGTTT | epegRNA with | spacer + scaffold + |
| CT2_GA2_ | | AAGAGCTAGAAATAGCAAGTTTAA | UA flip, 4CAG | flap + linker + 3′motif |
| GC3 | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGAAAG | | |
| | | GACTTGAGCGATTCAAAGGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GA1_ | 615 | GACCCTGGAAAAGCTGATGAGTTT | epegRNA with | spacer + scaffold + |
| CT2_GA2_ | | AAGAGCTAGAAATAGCAAGTTTAA | UA flip, 4CAG | flap + linker + 3′motif |
| GC4 | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGAAAG | | |
| | | GACTTGAGGGATTCAAACGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GC2_ | 616 | GACCCTGGAAAAGCTGATGAGTTT | epegRNA with | spacer + scaffold + |
| GC3_CT1_ | | AAGAGCTAGAAATAGCAAGTTTAA | UA flip, 4CAG | flap + linker + 3′motif |
| GA1 | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGAAAG | | |
| | | GATTTCAGCGACTCGAAGGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GC2_ | 617 | GACCCTGGAAAAGCTGATGAGTTT | epegRNA with | spacer + scaffold + |
| GC3_CT1_ | | AAGAGCTAGAAATAGCAAGTTTAA | UA flip, 4CAG | flap + linker + 3′motif |
| CT2 | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGGAAG | | |
| | | GATTTCAGCGATTCGAAGGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GC2_ | 618 | GACCCTGGAAAAGCTGATGAGTTT | epegRNA with | spacer + scaffold + |
| GC3_CT1_ | | AAGAGCTAGAAATAGCAAGTTTAA | UA flip, 4CAG | flap + linker + 3′motif |
| GA2 | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGGAAG | | |
| | | GATTTCAGCGACTCAAAGGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GC2_ | 619 | GACCCTGGAAAAGCTGATGAGTTT | epegRNA with | spacer + scaffold + |
| GC3_GA1_ | | AAGAGCTAGAAATAGCAAGTTTAA | UA flip, 4CAG | flap + linker + 3′motif |
| CT2 | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGAAAG | | |
| | | GACTTCAGCGATTCGAAGGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GC2_ | 620 | GACCCTGGAAAAGCTGATGAGTTT | epegRNA with | spacer + scaffold + |
| GC3_GA1_ | | AAGAGCTAGAAATAGCAAGTTTAA | UA flip, 4CAG | flap + linker + 3′motif |
| GA2 | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGAAAG | | |
| | | GACTTCAGCGACTCAAAGGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GC2_ | 621 | GACCCTGGAAAAGCTGATGAGTTT | epegRNA with | spacer + scaffold + |
| GC3_CT2_ | | AAGAGCTAGAAATAGCAAGTTTAA | UA flip, 4CAG | flap + linker + 3′motif |
| GA2 | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGGAAG | | |
| | | GACTTCAGCGATTCAAAGGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GC2_ | 622 | GACCCTGGAAAAGCTGATGAGTTT | epegRNA with | spacer + scaffold + |
| GC4_CT1_ | | AAGAGCTAGAAATAGCAAGTTTAA | UA flip, 4CAG | flap + linker + 3′motif |
| CT2 | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGGAAG | | |
| | | GATTTCAGGGATTCGAACGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GC2_ | 623 | GACCCTGGAAAAGCTGATGAGTTT | epegRNA with | spacer + scaffold + |
| GC4_CT1_ | | AAGAGCTAGAAATAGCAAGTTTAA | UA flip, 4CAG | flap + linker + 3′motif |
| GA2 | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGGAAG | | |
| | | GATTTCAGGGACTCAAACGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GC2_ | 624 | GACCCTGGAAAAGCTGATGAGTTT | epegRNA with | spacer + scaffold + |
| GC4_GA1_ | | AAGAGCTAGAAATAGCAAGTTTAA | UA flip, 4CAG | flap + linker + 3′motif |
| CT2 | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGAAAG | | |
| | | GACTTCAGGGATTCGAACGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GC2_ | 625 | GACCCTGGAAAAGCTGATGAGTTT | epegRNA with | spacer + scaffold + |
| GC4_GA1_ | | AAGAGCTAGAAATAGCAAGTTTAA | UA flip, 4CAG | flap + linker + 3′motif |
| GA2 | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGAAAG | | |
| | | GACTTCAGGGACTCAAACGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GC2_ | 624 | GACCCTGGAAAAGCTGATGAGTTT | epegRNA with | spacer + scaffold + |
| GC4_CT2_ | | AAGAGCTAGAAATAGCAAGTTTAA | UA flip, 4CAG | flap + linker + 3′motif |
| GA2 | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGAAAG | | |
| | | GACTTCAGGGATTCGAACGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GC2_ | 626 | GACCCTGGAAAAGCTGATGAGTTT | epegRNA with | spacer + scaffold + |
| CT1_GA1_ | | AAGAGCTAGAAATAGCAAGTTTAA | UA flip, 4CAG | flap + linker + 3′motif |
| GA2 | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGAAAG | | |
| | | GATTTCAGGGACTCAAAGGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GC2_ | 627 | GACCCTGGAAAAGCTGATGAGTTT | epegRNA with | spacer + scaffold + |
| CT1_CT2_ | | AAGAGCTAGAAATAGCAAGTTTAA | UA flip, 4CAG | flap + linker + 3′motif |
| GA2 | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGGAAG | | |
| | | GATTTCAGGGATTCAAAGGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GC3_ | 628 | GACCCTGGAAAAGCTGATGAGTTT | epegRNA with | spacer + scaffold + |
| GC4_GA1_ | | AAGAGCTAGAAATAGCAAGTTTAA | UA flip, 4CAG | flap + linker + 3′motif |
| CT2 | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGAAAG | | |
| | | GACTTGAGCGATTCGAACGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GC3_ | 629 | GACCCTGGAAAAGCTGATGAGTTT | epegRNA with | spacer + scaffold + |
| GC4_GA1_ | | AAGAGCTAGAAATAGCAAGTTTAA | UA flip, 4CAG | flap + linker + 3′motif |
| GA2 | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGAAAG | | |
| | | GACTTGAGCGACTCAAACGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GC3_ | 630 | GACCCTGGAAAAGCTGATGAGTTT | epegRNA with | spacer + scaffold + |
| GC4_CT2_ | | AAGAGCTAGAAATAGCAAGTTTAA | UA flip, 4CAG | flap + linker + 3′motif |
| GA2 | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGGAAG | | |
| | | GACTTGAGCGATTCAAACGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GC3_ | 631 | GACCCTGGAAAAGCTGATGAGTTT | epegRNA with | spacer + scaffold + |
| CT1_GA1_ | | AAGAGCTAGAAATAGCAAGTTTAA | UA flip, 4CAG | flap + linker + 3′motif |
| GA2 | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGAAAG | | |
| | | GATTTGAGCGACTCAAAGGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GC3_ | 632 | GACCCTGGAAAAGCTGATGAGTTT | epegRNA with | spacer + scaffold + |
| CT1_CT2_ | | AAGAGCTAGAAATAGCAAGTTTAA | UA flip, 4CAG | flap + linker + 3′motif |
| GA2 | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGGAAG | | |
| | | GATTTGAGCGATTCAAAGGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GC4_ | 633 | GACCCTGGAAAAGCTGATGAGTTT | epegRNA with | spacer + scaffold + |
| CT1_CT2_ | | AAGAGCTAGAAATAGCAAGTTTAA | UA flip, 4CAG | flap + linker + 3′motif |
| GA2 | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGGAAG | | |
| | | GATTTGAGGGATTCAAACGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GC2_ | 634 | GACCCTGGAAAAGCTGATGAGTTT | epegRNA with | spacer + scaffold + |
| GC3_GC4_ | | AAGAGCTAGAAATAGCAAGTTTAA | UA flip, 4CAG | flap + linker + 3′motif |
| CT2_GA1 | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGAAAG | | |
| | | GACTTCAGCGATTCGAACGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GC2_ | 635 | GACCCTGGAAAAGCTGATGAGTTT | epegRNA with | spacer + scaffold + |
| GC3_GC4_ | | AAGAGCTAGAAATAGCAAGTTTAA | UA flip, 4CAG | flap + linker + 3′motif |
| CT1_GA1 | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGAAAG | | |
| | | GATTTCAGCGACTCGAACGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GC2_ | 636 | GACCCTGGAAAAGCTGATGAGTTT | epegRNA with | spacer + scaffold + |
| GC3_GC4_ | | AAGAGCTAGAAATAGCAAGTTTAA | UA flip, 4CAG | flap + linker + 3′motif |
| CT1_CT2 | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGGAAG | | |
| | | GATTTCAGCGATTCGAACGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GC2_ | 637 | GACCCTGGAAAAGCTGATGAGTTT | epegRNA with | spacer + scaffold + |
| GC3_GC4_ | | AAGAGCTAGAAATAGCAAGTTTAA | UA flip, 4CAG | flap + linker + 3′motif |
| CT1_GA2 | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGGAAG | | |
| | | GATTTCAGCGACTCAAACGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GC2_ | 638 | GACCCTGGAAAAGCTGATGAGTTT | epegRNA with | spacer + scaffold + |
| GC4_CT1_ | | AAGAGCTAGAAATAGCAAGTTTAA | UA flip, 4CAG | flap + linker + 3′motif |
| GA1_CT2 | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGAAAG | | |
| | | GATTTCAGGGATTCGAACGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GC2_ | 639 | GACCCTGGAAAAGCTGATGAGTTT | epegRNA with | spacer + scaffold + |
| GC4_CT1_ | | AAGAGCTAGAAATAGCAAGTTTAA | UA flip, 4CAG | flap + linker + 3′motif |
| GA1_GA2 | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGAAAG | | |
| | | GATTTCAGGGACTCAAACGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GC2_ | 640 | GACCCTGGAAAAGCTGATGAGTTT | epegRNA with | spacer + scaffold + |
| CT1_GA1_ | | AAGAGCTAGAAATAGCAAGTTTAA | UA flip, 4CAG | flap + linker + 3′motif |
| CT2_GA2 | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGAAAG | | |
| | | GATTTCAGGGATTCAAAGGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GC2_ | 641 | GACCCTGGAAAAGCTGATGAGTTT | epegRNA with | spacer + scaffold + |
| CT1_GA1_ | | AAGAGCTAGAAATAGCAAGTTTAA | UA flip, 4CAG | flap + linker + 3′motif |
| CT2_GC3 | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGAAAG | | |
| | | GATTTCAGCGATTCGAAGGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GC2_ | 638 | GACCCTGGAAAAGCTGATGAGTTT | epegRNA with | spacer + scaffold + |
| CT1_GA1_ | | AAGAGCTAGAAATAGCAAGTTTAA | UA flip, 4CAG | flap + linker + 3′motif |
| CT2_GC4 | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGAAAG | | |
| | | GATTTCAGGGATTCGAACGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GC2_ | 642 | GACCCTGGAAAAGCTGATGAGTTT | epegRNA with | spacer + scaffold + |
| GA1_CT2_ | | AAGAGCTAGAAATAGCAAGTTTAA | UA flip, 4CAG | flap + linker + 3′motif |
| GA2_GC3 | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGAAAG | | |
| | | GACTTCAGCGATTCAAAGGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GC2_ | 643 | GACCCTGGAAAAGCTGATGAGTTT | epegRNA with | spacer + scaffold + |
| GA1_CT2_ | | AAGAGCTAGAAATAGCAAGTTTAA | UA flip, 4CAG | flap + linker + 3′motif |
| GA2_GC4 | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGAAAG | | |
| | | GACTTCAGGGATTCAAACGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GC2_ | 644 | GACCCTGGAAAAGCTGATGAGTTT | epegRNA with | spacer + scaffold + |
| GC3_GC4_ | | AAGAGCTAGAAATAGCAAGTTTAA | UA flip, 4CAG | flap + linker + 3′motif |
| GA1_GA2 | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGAAAG | | |
| | | GACTTCAGCGACTCAAACGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GC2_ | 645 | GACCCTGGAAAAGCTGATGAGTTT | epegRNA with | spacer + scaffold + |
| GC3_GC4_ | | AAGAGCTAGAAATAGCAAGTTTAA | UA flip, 4CAG | flap + linker + 3′motif |
| CT2_GA2 | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGGAAG | | |
| | | GACTTCAGCGATTCAAACGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GC3_ | 646 | GACCCTGGAAAAGCTGATGAGTTT | epegRNA with | spacer + scaffold + |
| GC4_CT1_ | | AAGAGCTAGAAATAGCAAGTTTAA | UA flip, 4CAG | flap + linker + 3′motif |
| GA1_GA2 | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGAAAG | | |
| | | GATTTGAGCGACTCAAACGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GC3_ | 647 | GACCCTGGAAAAGCTGATGAGTTT | epegRNA with | spacer + scaffold + |
| GC4_GA1_ | | AAGAGCTAGAAATAGCAAGTTTAA | UA flip, 4CAG | flap + linker + 3′motif |
| CT2_GA2 | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGAAAG | | |
| | | GACTTGAGTGACTCAAACGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GC4 | 648 | GACCCTGGAAAAGCTGATGAGTTT | epegRNA with | spacer + scaffold + |
| CT1_GA1 | | AAGAGCTAGAAATAGCAAGTTTAA | UA flip, 4CAG | flap + linker + 3′motif |
| CT2_GA2 | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGAAAG | | |
| | | GATTTGAGGGATTCAAACGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GC4_ | 638 | GACCCTGGAAAAGCTGATGAGTTT | epegRNA with | spacer + scaffold + |
| CT1_GA1_ | | AAGAGCTAGAAATAGCAAGTTTAA | UA flip, 4CAG | flap + linker + 3′motif |
| CT2_GC2 | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGAAAG | | |
| | | GATTTCAGGGATTCGAACGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GC4_ | 649 | GACCCTGGAAAAGCTGATGAGTTT | epegRNA with | spacer + scaffold + |
| CT1_GA1_ | | AAGAGCTAGAAATAGCAAGTTTAA | UA flip, 4CAG | flap + linker + 3′motif |
| CT2_GC3 | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGAAAG | | |
| | | GATTTGAGCGATTCGAACGCTTTC | | |
| | | ATCAGCTTTTCCAATCACTCGCGGT | | |
| | | TCTATCTAGTTACGCGTTAAACCA | | |
| | | ACTAGAA | | |
| htt_h31_CT1_ | 650 | GACCCTGGAAAAGCTGATGAGTTT | epegRNA with | spacer + scaffold + |
| GA1_CT2_ | | AAGAGCTAGAAATAGCAAGTTTAA | UA flip, 4CAG | flap + linker + 3′motif |
| GA2_GC3 | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGAAAG | | |
| | | GATTTGAGCGATTCAAAGGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GC2_ | 651 | GACCCTGGAAAAGCTGATGAGTTT | epegRNA with | spacer + scaffold + |
| GC4_CT1_ | | AAGAGCTAGAAATAGCAAGTTTAA | UA flip, 4CAG | flap + linker + 3′motif |
| CT2_GA2 | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGGAAG | | |
| | | GATTTCAGGGATTCAAACGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GC3_ | 646 | GACCCTGGAAAAGCTGATGAGTTT | epegRNA with | spacer + scaffold + |
| GC4_CT1_ | | AAGAGCTAGAAATAGCAAGTTTAA | UA flip, 4CAG | flap + linker + 3′motif |
| GA1_GA2 | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGAAAG | | |
| | | GATTTGAGCGACTCAAACGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GC3_ | 652 | GACCCTGGAAAAGCTGATGAGTTT | epegRNA with | spacer + scaffold + |
| GC4_CT1_ | | AAGAGCTAGAAATAGCAAGTTTAA | UA flip, 4CAG | flap + linker + 3′motif |
| CT2_GA2 | | ATAAGGCTAGTCCGTTATCAACTT | insertion (6Q) | |
| | | GAAAAAGTGGCACCGAGTCGGTGC | | |
| | | GCGGCGGCGGCGGCGGCGGTGGC | | |
| | | GGCTGTTGCTGCTGCTGCTGGAAG | | |
| | | GATTTGAGCGATTCAAACGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GC2_ | 653 | GACCCTGGAAAAGCTGATGAGTTT | epegRNA with | spacer + scaffold + |
| GC3_GC4_ | | AAGAGCTAGAAATAGCAAGTTTAA | UA flip, 4CAG | flap + linker + 3′motif |
| CT1_GA1_ | | ATAAGGCTAGTCCGTTATCAACTT | insertion (6Q) | |
| CT2 | | GAAAAAGTGGCACCGAGTCGGTGC | | |
| | | GCGGCGGCGGCGGCGGCGGTGGC | | |
| | | GGCTGTTGCTGCTGCTGCTGAAAG | | |
| | | GATTTCAGCGATTCGAACGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GC2_ | 654 | GACCCTGGAAAAGCTGATGAGTTT | epegRNA with | spacer + scaffold + |
| GC3_GC4_ | | AAGAGCTAGAAATAGCAAGTTTAA | UA flip, 4CAG | flap + linker + 3′motif |
| CT1_GA1_ | | ATAAGGCTAGTCCGTTATCAACTT | insertion (6Q) | |
| GA2 | | GAAAAAGTGGCACCGAGTCGGTGC | | |
| | | GCGGCGGCGGCGGCGGCGGTGGC | | |
| | | GGCTGTTGCTGCTGCTGCTGAAAG | | |
| | | GATTTCAGCGACTCAAACGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GC3_ | 655 | GACCCTGGAAAAGCTGATGAGTTT | epegRNA with | spacer + scaffold + |
| GC4_CT1_ | | AAGAGCTAGAAATAGCAAGTTTAA | UA flip, 4CAG | flap + linker + 3′motif |
| GA1_CT2_ | | ATAAGGCTAGTCCGTTATCAACTT | insertion (6Q) | |
| GA2 | | GAAAAAGTGGCACCGAGTCGGTGC | | |
| | | GCGGCGGCGGCGGCGGCGGTGGC | | |
| | | GGCTGTTGCTGCTGCTGCTGAAAG | | |
| | | GATTTGAGTGACTCAAACGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GC4_ | 656 | GACCCTGGAAAAGCTGATGAGTTT | epegRNA with | spacer + scaffold + |
| CT1_GA1_ | | AAGAGCTAGAAATAGCAAGTTTAA | UA flip, 4CAG | flap + linker + 3′motif |
| CT2_GA2_ | | ATAAGGCTAGTCCGTTATCAACTT | insertion (6Q) | |
| GC2 | | GAAAAAGTGGCACCGAGTCGGTGC | | |
| | | GCGGCGGCGGCGGCGGCGGTGGC | | |
| | | GGCTGTTGCTGCTGCTGCTGAAAG | | |
| | | GATTTCAGGGATTCAAACGCTTTC | | |
| | | ATCAGCTTTTCCAATCACTCGCGGT | | |
| | | TCTATCTAGTTACGCGTTAAACCA | | |
| | | ACTAGAA | | |
| htt_h31_CT1_ | 657 | GACCCTGGAAAAGCTGATGAGTTT | epegRNA with | spacer + scaffold + |
| GA1_CT2_ | | AAGAGCTAGAAATAGCAAGTTTAA | UA flip, 4CAG | flap + linker + 3′motif |
| GA2_GC2_ | | ATAAGGCTAGTCCGTTATCAACTT | insertion (6Q) | |
| GC3 | | GAAAAAGTGGCACCGAGTCGGTGC | | |
| | | GCGGCGGCGGCGGCGGCGGTGGC | | |
| | | GGCTGTTGCTGCTGCTGCTGAAAG | | |
| | | GATTTCAGCGATTCAAAGGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GA1_ | 656 | GACCCTGGAAAAGCTGATGAGTTT | epegRNA with | spacer + scaffold + |
| CT2_GA2_ | | AAGAGCTAGAAATAGCAAGTTTAA | UA flip, 4CAG | flap + linker + 3′motif |
| GC2_GC4_ | | ATAAGGCTAGTCCGTTATCAACTT | insertion (6Q) | |
| CT1 | | GAAAAAGTGGCACCGAGTCGGTGC | | |
| | | GCGGCGGCGGCGGCGGCGGTGGC | | |
| | | GGCTGTTGCTGCTGCTGCTGAAAG | | |
| | | GATTTCAGGGATTCAAACGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GC2_ | 658 | GACCCTGGAAAAGCTGATGAGTTT | epegRNA with | spacer + scaffold + |
| GC3_GC4_ | | AAGAGCTAGAAATAGCAAGTTTAA | UA flip, 4CAG | flap + linker + 3′motif |
| CT1_CT2_ | | ATAAGGCTAGTCCGTTATCAACTT | insertion (6Q) | |
| GA2 | | GAAAAAGTGGCACCGAGTCGGTGC | | |
| | | GCGGCGGCGGCGGCGGCGGTGGC | | |
| | | GGCTGTTGCTGCTGCTGCTGGAAG | | |
| | | GATTTCAGCGATTCAAACGCTTTC | | |
| | | ATCAGCTTTTCCAATCACTCGCGGT | | |
| | | TCTATCTAGTTACGCGTTAAACCA | | |
| | | ACTAGAA | | |
| htt_h31_GC2_ | 659 | GACCCTGGAAAAGCTGATGAGTTT | epegRNA with | spacer + scaffold + |
| GC3_GC4_ | | AAGAGCTAGAAATAGCAAGTTTAA | UA flip, 4CAG | flap + linker + 3′motif |
| CT1_GA1_ | | ATAAGGCTAGTCCGTTATCAACTT | insertion (6Q) | |
| CT2_GA2 | | GAAAAAGTGGCACCGAGTCGGTGC | | |
| | | GCGGCGGCGGCGGCGGCGGTGGC | | |
| | | GGCTGTTGCTGCTGCTGCTGAAAG | | |
| | | GATTTCAGCGATTCAAACGCTTTC | | |
| | | ATCAGCTTTTCCAATCACTCGCGGT | | |
| | | TCTATCTAGTTACGCGTTAAACCA | | |
| | | ACTAGAA | | |
| htt_h31_ | 660 | GACCCTGGAAAAGCTGATGAGTTT | epegRNA for | spacer + scaffold + |
| recoded | | AAGAGCTAGAAATAGCAAGTTTAA | various CAG | flap + linker + 3′motif |
| | | ATAAGGCTAGTCCGTTATCAACTT | replacement | |
| | | GAAAAAGTGGCACCGAGTCGGTGC | with UA flip | |
| | | GCGGCGGCGGCGGCGGCGGTGGC | | |
| | | GGCTGTTGCTGCTGCTGCTGAAAC | | |
| | | GATTTCAGCGATTCAAACGCTTTC | | |
| | | ATCAGCTTTTCCAATCACTCGCGGT | | |
| | | TCTATCTAGTTACGCGTTAAACCA | | |
| | | ACTAGAA | | |
| htt_h31_4CAR | 523 | GACCCTGGAAAAGCTGATGAGTTT | epegRNA for | spacer + scaffold + |
| | | AAGAGCTAGAAATAGCAAGTTTAA | various CAG | flap + linker + 3′motif |
| | | ATAAGGCTAGTCCGTTATCAACTT | replacement | |
| | | GAAAAAGTGGCACCGAGTCGGTGC | with UA flip | |
| | | GCGGCGGCGGCGGCGGCGGTGGC | | |
| | | GGCTGTTGCTGTTGCTGTTGGAAG | | |
| | | GACTTGAGGGACTCGAAGGCTTTC | | |
| | | ATCAGCTTTTCCAATCACTCGCGGT | | |
| | | TCTATCTAGTTACGCGTTAAACCA | | |
| | | ACTAGAA | | |
| htt_h31_2CAR | 522 | GACCCTGGAAAAGCTGATGAGTTT | epegRNA for | spacer + scaffold + |
| | | AAGAGCTAGAAATAGCAAGTTTAA | various CAG | flap + linker + 3′motif |
| | | ATAAGGCTAGTCCGTTATCAACTT | replacement | |
| | | GAAAAAGTGGCACCGAGTCGGTGC | with UA flip | |
| | | GCGGCGGCGGCGGCGGCGGTGGC | | |
| | | GGCTGTTGCTGTTGCTGCTGGAAG | | |
| | | GACTTGAGGGACTCGAAGGCTTTC | | |
| | | ATCAGCTTTTCCAATCACTCGCGGT | | |
| | | TCTATCTAGTTACGCGTTAAACCA | | |
| | | ACTAGAA | | |
| htt_h31_ | 526 | GACCCTGGAAAAGCTGATGAGTTT | epegRNA for | spacer + scaffold + |
| 4CAR_GC2 | | AAGAGCTAGAAATAGCAAGTTTAA | various CAG | flap + linker + 3′motif |
| | | ATAAGGCTAGTCCGTTATCAACTT | replacement | |
| | | GAAAAAGTGGCACCGAGTCGGTGC | with UA flip | |
| | | GCGGCGGCGGCGGCGGCGGTGGC | | |
| | | GGCTGTTGCTGTTGCTGTTGGAAG | | |
| | | GACTTCAGGGACTCGAAGGCTTTC | | |
| | | ATCAGCTTTTCCAATCACTCGCGGT | | |
| | | TCTATCTAGTTACGCGTTAAACCA | | |
| | | ACTAGAA | | |
| htt_h31_ | 525 | GACCCTGGAAAAGCTGATGAGTTT | epegRNA for | spacer + scaffold + |
| 2CAR_GC2 | | AAGAGCTAGAAATAGCAAGTTTAA | various CAG | flap + linker + 3′motif |
| | | ATAAGGCTAGTCCGTTATCAACTT | replacement | |
| | | GAAAAAGTGGCACCGAGTCGGTGC | with UA flip | |
| | | GCGGCGGCGGCGGCGGCGGTGGC | | |
| | | GGCTGTTGCTGTTGCTGCTGGAAG | | |
| | | GACTTCAGGGACTCGAAGGCTTTC | | |
| | | ATCAGCTTTTCCAATCACTCGCGGT | | |
| | | TCTATCTAGTTACGCGTTAAACCA | | |
| | | ACTAGAA | | |
| htt_h31_CAA | 524 | GACCCTGGAAAAGCTGATGAGTTT | epegRNA for | spacer + scaffold + |
| | | AAGAGCTAGAAATAGCAAGTTTAA | various CAG | flap + linker + 3′motif |
| | | ATAAGGCTAGTCCGTTATCAACTT | replacement | |
| | | GAAAAAGTGGCACCGAGTCGGTGC | with UA flip | |
| | | GCGGCGGCGGCGGCGGCGGTGGC | | |
| | | GGCTGTTGTTGTTGTTGTTGGAAG | | |
| | | GACTTGAGGGACTCGAAGGCTTTC | | |
| | | ATCAGCTTTTCCAATCACTCGCGGT | | |
| | | TCTATCTAGTTACGCGTTAAACCA | | |
| | | ACTAGAA | | |
| htt_h31_CAA | 528 | GACCCTGGAAAAGCTGATGAGTTT | epegRNA for | spacer + scaffold + |
| rec | | AAGAGCTAGAAATAGCAAGTTTAA | various CAG | flap + linker + 3′motif |
| | | ATAAGGCTAGTCCGTTATCAACTT | replacement | |
| | | GAAAAAGTGGCACCGAGTCGGTGC | with UA flip | |
| | | GCGGCGGCGGCGGCGGCGGTGGC | | |
| | | GGCTGTTGTTGTTGTTGTTGAAACG | | |
| | | ATTTCAGCGATTCAAACGCTTTCAT | | |
| | | CAGCTTTTCCAATCACTCGCGGTTC | | |
| | | TATCTAGTTACGCGTTAAACCAAC | | |
| | | TAGAA | | |
| htt_h31_CAA_ | 527 | GACCCTGGAAAAGCTGATGAGTTT | epegRNA for | spacer + scaffold + |
| GC2 | | AAGAGCTAGAAATAGCAAGTTTAA | various CAG | flap + linker + 3′motif |
| | | ATAAGGCTAGTCCGTTATCAACTT | replacement | |
| | | GAAAAAGTGGCACCGAGTCGGTGC | with UA flip | |
| | | GCGGCGGCGGCGGCGGCGGTGGC | | |
| | | GGCTGTTGTTGTTGTTGTTGGAAG | | |
| | | GACTTCAGGGACTCGAAGGCTTTC | | |
| | | ATCAGCTTTTCCAATCACTCGCGGT | | |
| | | TCTATCTAGTTACGCGTTAAACCA | | |
| | | ACTAGAA | | |
| htt_h40_GC3 | 661 | GACCCTGGAAAAGCTGATGAGTTT | epegRNA with | spacer + scaffold + |
| | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGGAAGGACTTGAGCGACTCG | | |
| | | AAGGCTTTCATCAGCTTTTCCAATC | | |
| | | ACTCGCGGTTCTATCTAGTTACGC | | |
| | | GTTAAACCAACTAGAA | | |
| htt_h33 | 662 | GACCCTGGAAAAGCTGATGAGTTT | epegRNA with | spacer + scaffold + |
| | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | CGGCGGCGGCGGCGGCGGCGGTG | | |
| | | GCGGCTGTTGCTGCTGCTGCTGGA | | |
| | | AGGACTTGAGGGACTCGAAGGCTT | | |
| | | TCATCAGCTTTTCCAATCACTCGCG | | |
| | | GTTCTATCTAGTTACGCGTTAAACC | | |
| | | AACTAGAA | | |
| htt_h33_GC2 | 663 | GACCCTGGAAAAGCTGATGAGTTT | epegRNA with | spacer + scaffold + |
| | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | CGGCGGCGGCGGCGGCGGCGGTG | | |
| | | GCGGCTGTTGCTGCTGCTGCTGGA | | |
| | | AGGACTTCAGGGACTCGAAGGCTT | | |
| | | TCATCAGCTTTTCCAATCACTCGCG | | |
| | | GTTCTATCTAGTTACGCGTTAAACC | | |
| | | AACTAGAA | | |
| htt_h33_CT2 | 664 | GACCCTGGAAAAGCTGATGAGTTT | epegRNA with | spacer + scaffold + |
| | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | CGGCGGCGGCGGCGGCGGCGGTG | | |
| | | GCGGCTGTTGCTGCTGCTGCTGGA | | |
| | | AGGACTTGAGGGATTCGAAGGCTT | | |
| | | TCATCAGCTTTTCCAATCACTCGCG | | |
| | | GTTCTATCTAGTTACGCGTTAAACC | | |
| | | AACTAGAA | | |
| htt_h33_CT2_ | 665 | GACCCTGGAAAAGCTGATGAGTTT | epegRNA with | spacer + scaffold + |
| GC2 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | CGGCGGCGGCGGCGGCGGCGGTG | | |
| | | GCGGCTGTTGCTGCTGCTGCTGGA | | |
| | | AGGACTTCAGGGATTCGAAGGCTT | | |
| | | TCATCAGCTTTTCCAATCACTCGCG | | |
| | | GTTCTATCTAGTTACGCGTTAAACC | | |
| | | AACTAGAA | | |
| htt_h40 | 519 | GACCCTGGAAAAGCTGATGAGTTT | epegRNA with | spacer + scaffold + |
| | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGGAAGGACTTGAGGGACTCG | | |
| | | AAGGCTTTCATCAGCTTTTCCAATC | | |
| | | ACTCGCGGTTCTATCTAGTTACGC | | |
| | | GTTAAACCAACTAGAA | | |
| htt_h40_G2toC | 666 | GACCCTGGAAAAGCTGATGAGTTT | epegRNA with | spacer + scaffold + |
| | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGGAAGGACTTGAGGGACTCG | | |
| | | AAGGCGTTCATCAGCTTTTCCAAT | | |
| | | CACTCGCGGTTCTATCTAGTTACGC | | |
| | | GTTAAACCAACTAGAA | | |
| htt_h40_G3toC | 667 | GACCCTGGAAAAGCTGATGAGTTT | epegRNA with | spacer + scaffold + |
| | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGGAAGGACTTGAGGGACTCG | | |
| | | AAGGGCTTCATCAGCTTTTCCAAT | | |
| | | CACTCGCGGTTCTATCTAGTTACGC | | |
| | | GTTAAACCAACTAGAA | | |
| htt_h40_2GtoC | 668 | GACCCTGGAAAAGCTGATGAGTTT | epegRNA with | spacer + scaffold + |
| | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGGAAGGACTTGAGGGACTCG | | |
| | | AAGGGGTTCATCAGCTTTTCCAAT | | |
| | | CACTCGCGGTTCTATCTAGTTACGC | | |
| | | GTTAAACCAACTAGAA | | |
| htt_h40_3GC | 669 | GACCCTGGAAAAGCTGATGAGTTT | epegRNA with | spacer + scaffold + |
| | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGGAACGACTTCAGCGACTCG | | |
| | | AAGGCTTTCATCAGCTTTTCCAATC | | |
| | | ACTCGCGGTTCTATCTAGTTACGC | | |
| | | GTTAAACCAACTAGAA | | |
| htt_h40_GC1 | 670 | GACCCTGGAAAAGCTGATGAGTTT | epegRNA with | spacer + scaffold + |
| | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGGAACGACTTGAGGGACTCG | | |
| | | AAGGCTTTCATCAGCTTTTCCAATC | | |
| | | ACTCGCGGTTCTATCTAGTTACGC | | |
| | | GTTAAACCAACTAGAA | | |
| htt_h40_GC3 | 661 | GACCCTGGAAAAGCTGATGAGTTT | epegRNA with | spacer + scaffold + |
| | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGGAAGGACTTGAGCGACTCG | | |
| | | AAGGCTTTCATCAGCTTTTCCAATC | | |
| | | ACTCGCGGTTCTATCTAGTTACGC | | |
| | | GTTAAACCAACTAGAA | | |
| htt_h40_CT1 | 671 | GACCCTGGAAAAGCTGATGAGTTT | epegRNA with | spacer + scaffold + |
| | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGGAAGGATTTGAGGGACTCG | | |
| | | AAGGCTTTCATCAGCTTTTCCAATC | | |
| | | ACTCGCGGTTCTATCTAGTTACGC | | |
| | | GTTAAACCAACTAGAA | | |
| htt_h40_GA1 | 672 | GACCCTGGAAAAGCTGATGAGTTT | epegRNA with | spacer + scaffold + |
| | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGAAAGGACTTGAGGGACTCG | | |
| | | AAGGCTTTCATCAGCTTTTCCAATC | | |
| | | ACTCGCGGTTCTATCTAGTTACGC | | |
| | | GTTAAACCAACTAGAA | | |
| htt_h40_CT2 | 673 | GACCCTGGAAAAGCTGATGAGTTT | epegRNA with | spacer + scaffold + |
| | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGGAAGGACTTGAGGGATTCG | | |
| | | AAGGCTTTCATCAGCTTTTCCAATC | | |
| | | ACTCGCGGTTCTATCTAGTTACGC | | |
| | | GTTAAACCAACTAGAA | | |
| htt_h40_GA2 | 674 | GACCCTGGAAAAGCTGATGAGTTT | epegRNA with | spacer + scaffold + |
| | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGGAAGGACTTGAGGGACTCA | | |
| | | AAGGCTTTCATCAGCTTTTCCAATC | | |
| | | ACTCGCGGTTCTATCTAGTTACGC | | |
| | | GTTAAACCAACTAGAA | | |
| htt_h40_GC4 | 675 | GACCCTGGAAAAGCTGATGAGTTT | epegRNA with | spacer + scaffold + |
| | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGGAAGGACTTGAGGGACTCG | | |
| | | AACGCTTTCATCAGCTTTTCCAATC | | |
| | | ACTCGCGGTTCTATCTAGTTACGC | | |
| | | GTTAAACCAACTAGAA | | |
| htt_h40_CT1_ | 676 | GACCCTGGAAAAGCTGATGAGTTT | epegRNA with | spacer + scaffold + |
| GC2 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGGAAGGATTTCAGGGACTCG | | |
| | | AAGGCTTTCATCAGCTTTTCCAATC | | |
| | | ACTCGCGGTTCTATCTAGTTACGC | | |
| | | GTTAAACCAACTAGAA | | |
| htt_h40_GA1_ | 677 | GACCCTGGAAAAGCTGATGAGTTT | epegRNA with | spacer + scaffold + |
| GC2 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGAAAGGACTTCAGGGACTCG | | |
| | | AAGGCTTTCATCAGCTTTTCCAATC | | |
| | | ACTCGCGGTTCTATCTAGTTACGC | | |
| | | GTTAAACCAACTAGAA | | |
| htt_h40_CT2_ | 678 | GACCCTGGAAAAGCTGATGAGTTT | epegRNA with | spacer + scaffold + |
| GC2 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGGAAGGACTTCAGGGATTCG | | |
| | | AAGGCTTTCATCAGCTTTTCCAATC | | |
| | | ACTCGCGGTTCTATCTAGTTACGC | | |
| | | GTTAAACCAACTAGAA | | |
| htt_h40_GA2_ | 679 | GACCCTGGAAAAGCTGATGAGTTT | epegRNA with | spacer + scaffold + |
| GC2 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGGAAGGACTTCAGGGACTCA | | |
| | | AAGGCTTTCATCAGCTTTTCCAATC | | |
| | | ACTCGCGGTTCTATCTAGTTACGC | | |
| | | GTTAAACCAACTAGAA | | |
| htt_h40_GC4_ | 680 | GACCCTGGAAAAGCTGATGAGTTT | epegRNA with | spacer + scaffold + |
| GC2 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGGAAGGACTTCAGGGACTCG | | |
| | | AACGCTTTCATCAGCTTTTCCAATC | | |
| | | ACTCGCGGTTCTATCTAGTTACGC | | |
| | | GTTAAACCAACTAGAA | | |
| htt_ | 681 | GACCCTGGAAAAGCTGATGAGTTT | epegRNA with | spacer + scaffold + |
| h40p10seed_n16eq | | AAGAGCTAGAAATAGCAAGTTTAA | UA flip, 4CAG | flap + linker + 3′motif |
| (GC2 + 3) | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGGAAGGACTTCAGCGACTCG | | |
| | | AAGGCTTTCATCAGCTTTTCCAATC | | |
| | | ACTCGCGGTTCTATCTAGTTACGC | | |
| | | GTTAAACCAACTAGAA | | |
| htt_ | 682 | GACCCTGGAAAAGCTGATGAGTTT | epegRNA with | spacer + scaffold + |
| h40p10seed_n7eq | | AAGAGCTAGAAATAGCAAGTTTAA | UA flip, 4CAG | flap + linker + 3′motif |
| (GC1 + 2) | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGGAACGACTTCAGGGACTCG | | |
| | | AAGGCTTTCATCAGCTTTTCCAATC | | |
| | | ACTCGCGGTTCTATCTAGTTACGC | | |
| | | GTTAAACCAACTAGAA | | |
| htt_h40_CT1_ | 683 | GACCCTGGAAAAGCTGATGAGTTT | epegRNA with | spacer + scaffold + |
| GA1 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGAAAGGATTTGAGGGACTCG | | |
| | | AAGGCTTTCATCAGCTTTTCCAATC | | |
| | | ACTCGCGGTTCTATCTAGTTACGC | | |
| | | GTTAAACCAACTAGAA | | |
| htt_h40_CT1_ | 684 | GACCCTGGAAAAGCTGATGAGTTT | epegRNA with | spacer + scaffold + |
| CT2 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGGAAGGATTTGAGGGATTCG | | |
| | | AAGGCTTTCATCAGCTTTTCCAATC | | |
| | | ACTCGCGGTTCTATCTAGTTACGC | | |
| | | GTTAAACCAACTAGAA | | |
| htt_h40_CT1_ | 685 | GACCCTGGAAAAGCTGATGAGTTT | epegRNA with | spacer + scaffold + |
| GA2 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGGAAGGATTTGAGGGACTCA | | |
| | | AAGGCTTTCATCAGCTTTTCCAATC | | |
| | | ACTCGCGGTTCTATCTAGTTACGC | | |
| | | GTTAAACCAACTAGAA | | |
| htt_h40_CT1_ | 686 | GACCCTGGAAAAGCTGATGAGTTT | epegRNA with | spacer + scaffold + |
| GC4 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGGAAGGATTTGAGGGACTCG | | |
| | | AACGCTTTCATCAGCTTTTCCAATC | | |
| | | ACTCGCGGTTCTATCTAGTTACGC | | |
| | | GTTAAACCAACTAGAA | | |
| htt_h40_GA1_ | 687 | GACCCTGGAAAAGCTGATGAGTTT | epegRNA with | spacer + scaffold + |
| CT2 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGAAAGGACTTGAGGGATTCG | | |
| | | AAGGCTTTCATCAGCTTTTCCAATC | | |
| | | ACTCGCGGTTCTATCTAGTTACGC | | |
| | | GTTAAACCAACTAGAA | | |
| htt_h40_GA1_ | 688 | GACCCTGGAAAAGCTGATGAGTTT | epegRNA with | spacer + scaffold + |
| GA2 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGAAAGGACTTGAGGGACTCA | | |
| | | AAGGCTTTCATCAGCTTTTCCAATC | | |
| | | ACTCGCGGTTCTATCTAGTTACGC | | |
| | | GTTAAACCAACTAGAA | | |
| htt_h40_GA1_ | 689 | GACCCTGGAAAAGCTGATGAGTTT | epegRNA with | spacer + scaffold + |
| GC4 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGAAAGGACTTGAGGGACTCG | | |
| | | AACGCTTTCATCAGCTTTTCCAATC | | |
| | | ACTCGCGGTTCTATCTAGTTACGC | | |
| | | GTTAAACCAACTAGAA | | |
| htt_h40_CT2_ | 690 | GACCCTGGAAAAGCTGATGAGTTT | epegRNA with | spacer + scaffold + |
| GA2 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGGAAGGACTTGAGGGATTCA | | |
| | | AAGGCTTTCATCAGCTTTTCCAATC | | |
| | | ACTCGCGGTTCTATCTAGTTACGC | | |
| | | GTTAAACCAACTAGAA | | |
| htt_h40_CT2_ | 691 | GACCCTGGAAAAGCTGATGAGTTT | epegRNA with | spacer + scaffold + |
| GC4 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGGAAGGACTTGAGGGATTCG | | |
| | | AACGCTTTCATCAGCTTTTCCAATC | | |
| | | ACTCGCGGTTCTATCTAGTTACGC | | |
| | | GTTAAACCAACTAGAA | | |
| htt_h40_GA2_ | 692 | GACCCTGGAAAAGCTGATGAGTTT | epegRNA with | spacer + scaffold + |
| GC4 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGGAAGGACTTGAGGGACTCA | | |
| | | AACGCTTTCATCAGCTTTTCCAATC | | |
| | | ACTCGCGGTTCTATCTAGTTACGC | | |
| | | GTTAAACCAACTAGAA | | |
| htt_h40_GC3_ | 693 | GACCCTGGAAAAGCTGATGAGTTT | epegRNA with | spacer + scaffold + |
| CT1 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGGAAGGATTTGAGCGACTCG | | |
| | | AAGGCTTTCATCAGCTTTTCCAATC | | |
| | | ACTCGCGGTTCTATCTAGTTACGC | | |
| | | GTTAAACCAACTAGAA | | |
| htt_h40_GC3_ | 694 | GACCCTGGAAAAGCTGATGAGTTT | epegRNA with | spacer + scaffold + |
| GA1 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGAAAGGACTTGAGCGACTCG | | |
| | | AAGGCTTTCATCAGCTTTTCCAATC | | |
| | | ACTCGCGGTTCTATCTAGTTACGC | | |
| | | GTTAAACCAACTAGAA | | |
| htt_h40_GC3_ | 695 | GACCCTGGAAAAGCTGATGAGTTT | epegRNA with | spacer + scaffold + |
| CT2 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGGAAGGACTTGAGCGATTCG | | |
| | | AAGGCTTTCATCAGCTTTTCCAATC | | |
| | | ACTCGCGGTTCTATCTAGTTACGC | | |
| | | GTTAAACCAACTAGAA | | |
| htt_h40_GC3_ | 696 | GACCCTGGAAAAGCTGATGAGTTT | epegRNA with | spacer + scaffold + |
| GA2 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGGAAGGACTTGAGCGACTCA | | |
| | | AAGGCTTTCATCAGCTTTTCCAATC | | |
| | | ACTCGCGGTTCTATCTAGTTACGC | | |
| | | GTTAAACCAACTAGAA | | |
| htt_h40_GC3_ | 697 | GACCCTGGAAAAGCTGATGAGTTT | epegRNA with | spacer + scaffold + |
| GC4 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGGAAGGACTTGAGCGACTCG | | |
| | | AACGCTTTCATCAGCTTTTCCAATC | | |
| | | ACTCGCGGTTCTATCTAGTTACGC | | |
| | | GTTAAACCAACTAGAA | | |
| htt_h40_GC2_ | 698 | GACCCTGGAAAAGCTGATGAGTTT | epegRNA with | spacer + scaffold + |
| GC3_CT2 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGGAAGGACTTCAGCGATTCG | | |
| | | AAGGCTTTCATCAGCTTTTCCAATC | | |
| | | ACTCGCGGTTCTATCTAGTTACGC | | |
| | | GTTAAACCAACTAGAA | | |
| htt_h40_GC2_ | 699 | GACCCTGGAAAAGCTGATGAGTTT | epegRNA with | spacer + scaffold + |
| GC3_GC4 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGGAAGGACTTCAGCGACTCG | | |
| | | AACGCTTTCATCAGCTTTTCCAATC | | |
| | | ACTCGCGGTTCTATCTAGTTACGC | | |
| | | GTTAAACCAACTAGAA | | |
| htt_h40_GC2_ | 700 | GACCCTGGAAAAGCTGATGAGTTT | epegRNA with | spacer + scaffold + |
| GC3_CT1 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGGAAGGATTTCAGCGACTCG | | |
| | | AAGGCTTTCATCAGCTTTTCCAATC | | |
| | | ACTCGCGGTTCTATCTAGTTACGC | | |
| | | GTTAAACCAACTAGAA | | |
| htt_h40_GC2_ | 701 | GACCCTGGAAAAGCTGATGAGTTT | epegRNA with | spacer + scaffold + flap |
| GC3_GA1 | | AAGAGCTAGAAATAGCAAGTTTAA | UA flip, 4CAG | + linker + 3′motif |
| | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGAAAGGACTTCAGCGACTCG | | |
| | | AAGGCTTTCATCAGCTTTTCCAATC | | |
| | | ACTCGCGGTTCTATCTAGTTACGC | | |
| | | GTTAAACCAACTAGAA | | |
| htt_h40_GC2_ | 702 | GACCCTGGAAAAGCTGATGAGTTT | epegRNA with | spacer + scaffold + |
| GC3_GA2 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGGAAGGACTTCAGCGACTCA | | |
| | | AAGGCTTTCATCAGCTTTTCCAATC | | |
| | | ACTCGCGGTTCTATCTAGTTACGC | | |
| | | GTTAAACCAACTAGAA | | |
| htt_h40_CT1_ | 703 | GACCCTGGAAAAGCTGATGAGTTT | epegRNA with | spacer + scaffold + |
| GA1_CT2 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGAAAGGATTTGAGGGATTCG | | |
| | | AAGGCTTTCATCAGCTTTTCCAATC | | |
| | | ACTCGCGGTTCTATCTAGTTACGC | | |
| | | GTTAAACCAACTAGAA | | |
| htt_h40_CT1_ | 704 | GACCCTGGAAAAGCTGATGAGTTT | epegRNA with | spacer + scaffold + |
| GA1_GA2 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGAAAGGATTTGAGGGACTCA | | |
| | | AAGGCTTTCATCAGCTTTTCCAATC | | |
| | | ACTCGCGGTTCTATCTAGTTACGC | | |
| | | GTTAAACCAACTAGAA | | |
| htt_h40_CT1_ | 705 | GACCCTGGAAAAGCTGATGAGTTT | epegRNA with | spacer + scaffold + |
| GA1_GC4 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGAAAGGATTTGAGGGACTCG | | |
| | | AAcGCTTTCATCAGCTTTTCCAATC | | |
| | | ACTCGCGGTTCTATCTAGTTACGC | | |
| | | GTTAAACCAACTAGAA | | |
| htt_h40_CT1_ | 706 | GACCCTGGAAAAGCTGATGAGTTT | epegRNA with | spacer + scaffold + |
| GA1_GC2 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGAAAGGATTTCAGGGACTCG | | |
| | | AAGGCTTTCATCAGCTTTTCCAATC | | |
| | | ACTCGCGGTTCTATCTAGTTACGC | | |
| | | GTTAAACCAACTAGAA | | |
| htt_h40_GA1_ | 707 | GACCCTGGAAAAGCTGATGAGTTT | epegRNA with | spacer + scaffold + |
| CT2_GA2 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGAAAGGACTTGAGGGATTCA | | |
| | | AAGGCTTTCATCAGCTTTTCCAATC | | |
| | | ACTCGCGGTTCTATCTAGTTACGC | | |
| | | GTTAAACCAACTAGAA | | |
| htt_h40_GA1_ | 708 | GACCCTGGAAAAGCTGATGAGTTT | epegRNA with | spacer + scaffold + |
| CT2_GC4 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGAAAGGACTTGAGGGATTCG | | |
| | | AACGCTTTCATCAGCTTTTCCAATC | | |
| | | ACTCGCGGTTCTATCTAGTTACGC | | |
| | | GTTAAACCAACTAGAA | | |
| htt_h40_GA1_ | 709 | GACCCTGGAAAAGCTGATGAGTTT | epegRNA with | spacer + scaffold + |
| CT2_GC2 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGAAAGGACTTCAGGGATTCG | | |
| | | AAGGCTTTCATCAGCTTTTCCAATC | | |
| | | ACTCGCGGTTCTATCTAGTTACGC | | |
| | | GTTAAACCAACTAGAA | | |
| htt_h40_CT2_ | 691 | GACCCTGGAAAAGCTGATGAGTTT | epegRNA with | spacer + scaffold + |
| GA2_GC4 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGGAAGGACTTGAGGGATTCG | | |
| | | AACGCTTTCATCAGCTTTTCCAATC | | |
| | | ACTCGCGGTTCTATCTAGTTACGC | | |
| | | GTTAAACCAACTAGAA | | |
| htt_h40_CT2_ | 678 | GACCCTGGAAAAGCTGATGAGTTT | epegRNA with | spacer + scaffold + |
| GA2_GC2 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGGAAGGACTTCAGGGATTCG | | |
| | | AAGGCTTTCATCAGCTTTTCCAATC | | |
| | | ACTCGCGGTTCTATCTAGTTACGC | | |
| | | GTTAAACCAACTAGAA | | |
| htt_h40_GA2_ | 710 | GACCCTGGAAAAGCTGATGAGTTT | epegRNA with | spacer + scaffold + |
| GC4_GC2 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGGAAGGACTTCAGGGACTCA | | |
| | | AACGCTTTCATCAGCTTTTCCAATC | | |
| | | ACTCGCGGTTCTATCTAGTTACGC | | |
| | | GTTAAACCAACTAGAA | | |
| htt_h40_CT1_ | 703 | GACCCTGGAAAAGCTGATGAGTTT | epegRNA with | spacer + scaffold + |
| CT2_GA2 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGAAAGGATTTGAGGGATTCG | | |
| | | AAGGCTTTCATCAGCTTTTCCAATC | | |
| | | ACTCGCGGTTCTATCTAGTTACGC | | |
| | | GTTAAACCAACTAGAA | | |
| htt_h40_CT1_ | 711 | GACCCTGGAAAAGCTGATGAGTTT | epegRNA with | spacer + scaffold + |
| CT2_GC4 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGGAAGGATTTGAGGGATTCG | | |
| | | AACGCTTTCATCAGCTTTTCCAATC | | |
| | | ACTCGCGGTTCTATCTAGTTACGC | | |
| | | GTTAAACCAACTAGAA | | |
| htt_h40_CT1_ | 712 | GACCCTGGAAAAGCTGATGAGTTT | epegRNA with | spacer + scaffold + |
| CT2_GC2 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGGAAGGATTTCAGGGATTCG | | |
| | | AAGGCTTTCATCAGCTTTTCCAATC | | |
| | | ACTCGCGGTTCTATCTAGTTACGC | | |
| | | GTTAAACCAACTAGAA | | |
| htt_h40_CT1_ | 713 | GACCCTGGAAAAGCTGATGAGTTT | epegRNA with | spacer + scaffold + |
| GA2_GC4 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGGAAGGATTTGAGGGACTCA | | |
| | | AACGCTTTCATCAGCTTTTCCAATC | | |
| | | ACTCGCGGTTCTATCTAGTTACGC | | |
| | | GTTAAACCAACTAGAA | | |
| htt_h40_CT1_ | 706 | GACCCTGGAAAAGCTGATGAGTTT | epegRNA with | spacer + scaffold + |
| GA2_GC2 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGAAAGGATTTCAGGGACTCG | | |
| | | AAGGCTTTCATCAGCTTTTCCAATC | | |
| | | ACTCGCGGTTCTATCTAGTTACGC | | |
| | | GTTAAACCAACTAGAA | | |
| htt_h40_CT1_ | 714 | GACCCTGGAAAAGCTGATGAGTTT | epegRNA with | spacer + scaffold + |
| GC3_CT2 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGGAAGGATTTGAGCGATTCG | | |
| | | AAGGCTTTCATCAGCTTTTCCAATC | | |
| | | ACTCGCGGTTCTATCTAGTTACGC | | |
| | | GTTAAACCAACTAGAA | | |
| htt_h40_GA1_ | 715 | GACCCTGGAAAAGCTGATGAGTTT | UA flip, 4CAG | spacer + scaffold + |
| GC3_CT2 | | AAGAGCTAGAAATAGCAAGTTTAA | epegRNA with | flap + linker + 3′motif |
| | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGAAAGGACTTGAGCGATTCG | | |
| | | AAGGCTTTCATCAGCTTTTCCAATC | | |
| | | ACTCGCGGTTCTATCTAGTTACGC | | |
| | | GTTAAACCAACTAGAA | | |
| htt_h40_GA2_ | 716 | GACCCTGGAAAAGCTGATGAGTTT | epegRNA with | spacer + scaffold + |
| GC3_CT2 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGGAAGGACTTGAGCGATTCA | | |
| | | AAGGCTTTCATCAGCTTTTCCAATC | | |
| | | ACTCGCGGTTCTATCTAGTTACGC | | |
| | | GTTAAACCAACTAGAA | | |
| htt_h40_CT1_ | 717 | GACCCTGGAAAAGCTGATGAGTTT | epegRNA with | spacer + scaffold + |
| GC3_GC4 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGGAAGGATTTGAGCGACTCG | | |
| | | AACGCTTTCATCAGCTTTTCCAATC | | |
| | | ACTCGCGGTTCTATCTAGTTACGC | | |
| | | GTTAAACCAACTAGAA | | |
| htt_h40_GA1_ | 718 | GACCCTGGAAAAGCTGATGAGTTT | epegRNA with | spacer + scaffold + |
| GC3_GC4 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGAAAGGACTTGAGCGACTCG | | |
| | | AACGCTTTCATCAGCTTTTCCAATC | | |
| | | ACTCGCGGTTCTATCTAGTTACGC | | |
| | | GTTAAACCAACTAGAA | | |
| htt_h40_CT2_ | 719 | GACCCTGGAAAAGCTGATGAGTTT | epegRNA with | spacer + scaffold + |
| GC3_GC4 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGGAAGGACTTGAGCGATTCG | | |
| | | AACGCTTTCATCAGCTTTTCCAATC | | |
| | | ACTCGCGGTTCTATCTAGTTACGC | | |
| | | GTTAAACCAACTAGAA | | |
| htt_h40_GA2_ | 720 | GACCCTGGAAAAGCTGATGAGTTT | epegRNA with | spacer + scaffold + |
| GC3_GC4 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGGAAGGACTTGAGCGACTCA | | |
| | | AACGCTTTCATCAGCTTTTCCAATC | | |
| | | ACTCGCGGTTCTATCTAGTTACGC | | |
| | | GTTAAACCAACTAGAA | | |
| htt_h40_GA1_ | 721 | GACCCTGGAAAAGCTGATGAGTTT | epegRNA with | spacer + scaffold + |
| GC3_CT1 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGAAAGGATTTGAGCGACTCG | | |
| | | AAGGCTTTCATCAGCTTTTCCAATC | | |
| | | ACTCGCGGTTCTATCTAGTTACGC | | |
| | | GTTAAACCAACTAGAA | | |
| htt_h40_GA2_ | 722 | GACCCTGGAAAAGCTGATGAGTTT | epegRNA with | spacer + scaffold + |
| GC3_CT1 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGGAAGGATTTGAGCGACTCA | | |
| | | AAGGCTTTCATCAGCTTTTCCAATC | | |
| | | ACTCGCGGTTCTATCTAGTTACGC | | |
| | | GTTAAACCAACTAGAA | | |
| htt_h40_GC2_ | 723 | GACCCTGGAAAAGCTGATGAGTTT | epegRNA with | spacer + scaffold + |
| GC4_CT1 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGGAAGGATTTCAGGGACTCG | | |
| | | AACGCTTTCATCAGCTTTTCCAATC | | |
| | | ACTCGCGGTTCTATCTAGTTACGC | | |
| | | GTTAAACCAACTAGAA | | |
| htt_h40_GC2_ | 724 | GACCCTGGAAAAGCTGATGAGTTT | epegRNA with | spacer + scaffold + |
| GC4_GA1 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGAAAGGACTTCAGGGACTCG | | |
| | | AACGCTTTCATCAGCTTTTCCAATC | | |
| | | ACTCGCGGTTCTATCTAGTTACGC | | |
| | | GTTAAACCAACTAGAA | | |
| htt_h40_GC2_ | 725 | GACCCTGGAAAAGCTGATGAGTTT | epegRNA with | spacer + scaffold + |
| GC4_CT2 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGGAAGGACTTCAGGGATTCG | | |
| | | AACGCTTTCATCAGCTTTTCCAATC | | |
| | | ACTCGCGGTTCTATCTAGTTACGC | | |
| | | GTTAAACCAACTAGAA | | |
| htt_h40_GC2_ | 726 | GACCCTGGAAAAGCTGATGAGTTT | epegRNA with | spacer + scaffold + |
| GA1_GA2 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGAAAGGACTTCAGGGACTCA | | |
| | | AAGGCTTTCATCAGCTTTTCCAATC | | |
| | | ACTCGCGGTTCTATCTAGTTACGC | | |
| | | GTTAAACCAACTAGAA | | |
| htt_h40_GC3_ | 727 | GACCCTGGAAAAGCTGATGAGTTT | epegRNA with | spacer + scaffold + |
| GA1_GA2 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGAAAGGACTTGAGCGACTCA | | |
| | | AAGGCTTTCATCAGCTTTTCCAATC | | |
| | | ACTCGCGGTTCTATCTAGTTACGC | | |
| | | GTTAAACCAACTAGAA | | |
| htt_h40_GC4_ | 728 | GACCCTGGAAAAGCTGATGAGTTT | epegRNA with | spacer + scaffold + |
| GA1_GA2 | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGAAAGGACTTGAGGGACTCA | | |
| | | AACGCTTTCATCAGCTTTTCCAATC | | |
| | | ACTCGCGGTTCTATCTAGTTACGC | | |
| | | GTTAAACCAACTAGAA | | |
| htt_h40_GC2_ | 729 | GACCCTGGAAAAGCTGATGAGTTT | epegRNA with | spacer + scaffold + |
| GC3_GC4_ | | AAGAGCTAGAAATAGCAAGTTTAA | UA flip, 4CAG | flap + linker + 3′motif |
| CT2 | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGGAAGGACTTCAGCGATTCG | | |
| | | AACGCTTTCATCAGCTTTTCCAATC | | |
| | | ACTCGCGGTTCTATCTAGTTACGC | | |
| | | GTTAAACCAACTAGAA | | |
| htt_h40_GC2_ | 730 | GACCCTGGAAAAGCTGATGAGTTT | epegRNA with | spacer + scaffold + |
| GC3_GC4_ | | AAGAGCTAGAAATAGCAAGTTTAA | UA flip, 4CAG | flap + linker + 3′motif |
| CT1 | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGGAAGGATTTCAGCGACTCG | | |
| | | AACGCTTTCATCAGCTTTTCCAATC | | |
| | | ACTCGCGGTTCTATCTAGTTACGC | | |
| | | GTTAAACCAACTAGAA | | |
| htt_h40_GC2_ | 731 | GACCCTGGAAAAGCTGATGAGTTT | epegRNA with | spacer + scaffold + |
| GC3_GC4_ | | AAGAGCTAGAAATAGCAAGTTTAA | UA flip, 4CAG | flap + linker + 3′motif |
| GA1 | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGAAAGGACTTCAGCGACTCG | | |
| | | AACGCTTTCATCAGCTTTTCCAATC | | |
| | | ACTCGCGGTTCTATCTAGTTACGC | | |
| | | GTTAAACCAACTAGAA | | |
| htt_h40_GC2_ | 732 | GACCCTGGAAAAGCTGATGAGTTT | epegRNA with | spacer + scaffold + |
| GC3_GC4_ | | AAGAGCTAGAAATAGCAAGTTTAA | UA flip, 4CAG | flap + linker + 3′motif |
| GA2 | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGGAAGGACTTCAGCGACTCA | | |
| | | AACGCTTTCATCAGCTTTTCCAATC | | |
| | | ACTCGCGGTTCTATCTAGTTACGC | | |
| | | GTTAAACCAACTAGAA | | |
| htt_h40_GC3_ | 733 | GACCCTGGAAAAGCTGATGAGTTT | epegRNA with | spacer + scaffold + |
| GC4_CT1_ | | AAGAGCTAGAAATAGCAAGTTTAA | UA flip, 4CAG | flap + linker + 3′motif |
| GA1 | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGAAAGGATTTGAGCGACTCG | | |
| | | AACGCTTTCATCAGCTTTTCCAATC | | |
| | | ACTCGCGGTTCTATCTAGTTACGC | | |
| | | GTTAAACCAACTAGAA | | |
| htt_h40_GC3_ | 734 | GACCCTGGAAAAGCTGATGAGTTT | epegRNA with | spacer + scaffold + |
| GC4_CT1_ | | AAGAGCTAGAAATAGCAAGTTTAA | UA flip, 4CAG | flap + linker + 3′motif |
| CT2 | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGGAAGGATTTGAGCGATTCG | | |
| | | AACGCTTTCATCAGCTTTTCCAATC | | |
| | | ACTCGCGGTTCTATCTAGTTACGC | | |
| | | GTTAAACCAACTAGAA | | |
| htt_h40_GC3_ | 733 | GACCCTGGAAAAGCTGATGAGTTT | epegRNA with | spacer + scaffold + |
| GC4_CT1_ | | AAGAGCTAGAAATAGCAAGTTTAA | UA flip, 4CAG | flap + linker + 3′motif |
| GA2 | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGAAAGGATTTGAGCGACTCG | | |
| | | AACGCTTTCATCAGCTTTTCCAATC | | |
| | | ACTCGCGGTTCTATCTAGTTACGC | | |
| | | GTTAAACCAACTAGAA | | |
| htt_h40_GC4_ | 735 | GACCCTGGAAAAGCTGATGAGTTT | epegRNA with | spacer + scaffold + |
| CT1_GA1_ | | AAGAGCTAGAAATAGCAAGTTTAA | UA flip, 4CAG | flap + linker + 3′motif |
| CT2 | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGAAAGGATTTGAGGGATTCG | | |
| | | AAcGCTTTCATCAGCTTTTCCAATC | | |
| | | ACTCGCGGTTCTATCTAGTTACGC | | |
| | | GTTAAACCAACTAGAA | | |
| htt_h40_GC4_ | 736 | GACCCTGGAAAAGCTGATGAGTTT | epegRNA with | spacer + scaffold + |
| CT1_GA1_ | | AAGAGCTAGAAATAGCAAGTTTAA | UA flip, 4CAG | flap + linker + 3′motif |
| GA2 | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGAAAGGATTTGAGGGACTCA | | |
| | | AAcGCTTTCATCAGCTTTTCCAATC | | |
| | | ACTCGCGGTTCTATCTAGTTACGC | | |
| | | GTTAAACCAACTAGAA | | |
| htt_h40_GC4_ | 737 | GACCCTGGAAAAGCTGATGAGTTT | epegRNA with | spacer + scaffold + |
| CT1_GA1_ | | AAGAGCTAGAAATAGCAAGTTTAA | UA flip, 4CAG | flap + linker + 3′motif |
| GC2 | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGAAAGGATTTCAGGGACTCG | | |
| | | AAcGCTTTCATCAGCTTTTCCAATC | | |
| | | ACTCGCGGTTCTATCTAGTTACGC | | |
| | | GTTAAACCAACTAGAA | | |
| htt_h40_CT1_ | 738 | GACCCTGGAAAAGCTGATGAGTTT | epegRNA with | spacer + scaffold + |
| GA1_CT2_ | | AAGAGCTAGAAATAGCAAGTTTAA | UA flip, 4CAG | flap + linker + 3′motif |
| GA2 | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGAAAGGATTTGAGGGATTCA | | |
| | | AAGGCTTTCATCAGCTTTTCCAATC | | |
| | | ACTCGCGGTTCTATCTAGTTACGC | | |
| | | GTTAAACCAACTAGAA | | |
| htt_h40_CT1_ | 739 | GACCCTGGAAAAGCTGATGAGTTT | epegRNA with | spacer + scaffold + |
| GA1_CT2_ | | AAGAGCTAGAAATAGCAAGTTTAA | UA flip, 4CAG | flap + linker + 3′motif |
| GC2 | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGAAAGGATTTCAGGGATTCG | | |
| | | AAGGCTTTCATCAGCTTTTCCAATC | | |
| | | ACTCGCGGTTCTATCTAGTTACGC | | |
| | | GTTAAACCAACTAGAA | | |
| htt_h40_CT1_ | 740 | GACCCTGGAAAAGCTGATGAGTTT | epegRNA with | spacer + scaffold + |
| GA1_CT2_ | | AAGAGCTAGAAATAGCAAGTTTAA | UA flip, 4CAG | flap + linker + 3′motif |
| GC3 | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGAAAGGATTTGAGCGATTCG | | |
| | | AAGGCTTTCATCAGCTTTTCCAATC | | |
| | | ACTCGCGGTTCTATCTAGTTACGC | | |
| | | GTTAAACCAACTAGAA | | |
| htt_h40_GA1_ | 741 | GACCCTGGAAAAGCTGATGAGTTT | epegRNA with | spacer + scaffold + |
| CT2_GA2_ | | AAGAGCTAGAAATAGCAAGTTTAA | UA flip, 4CAG | flap + linker + 3′motif |
| GC2 | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGAAAGGACTTCAGGGATTCA | | |
| | | AAGGCTTTCATCAGCTTTTCCAATC | | |
| | | ACTCGCGGTTCTATCTAGTTACGC | | |
| | | GTTAAACCAACTAGAA | | |
| htt_h40_GA1_ | 742 | GACCCTGGAAAAGCTGATGAGTTT | epegRNA with | spacer + scaffold + |
| CT2_GA2_ | | AAGAGCTAGAAATAGCAAGTTTAA | UA flip, 4CAG | flap + linker + 3′motif |
| GC3 | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGAAAGGACTTGAGCGATTCA | | |
| | | AAGGCTTTCATCAGCTTTTCCAATC | | |
| | | ACTCGCGGTTCTATCTAGTTACGC | | |
| | | GTTAAACCAACTAGAA | | |
| htt_h40_GA1_ | 743 | GACCCTGGAAAAGCTGATGAGTTT | epegRNA with | spacer + scaffold + |
| CT2_GA2_ | | AAGAGCTAGAAATAGCAAGTTTAA | UA flip, 4CAG | flap + linker + 3′motif |
| GC4 | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGAAAGGACTTGAGGGATTCA | | |
| | | AACGCTTTCATCAGCTTTTCCAATC | | |
| | | ACTCGCGGTTCTATCTAGTTACGC | | |
| | | GTTAAACCAACTAGAA | | |
| htt_h40_GC2_ | 744 | GACCCTGGAAAAGCTGATGAGTTT | epegRNA with | spacer + scaffold + |
| GC3_CT1_ | | AAGAGCTAGAAATAGCAAGTTTAA | UA flip, 4CAG | flap + linker + 3′motif |
| GA1 | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGAAAGGATTTCAGCGACTCG | | |
| | | AAGGCTTTCATCAGCTTTTCCAATC | | |
| | | ACTCGCGGTTCTATCTAGTTACGC | | |
| | | GTTAAACCAACTAGAA | | |
| htt_h40_GC2_ | 745 | GACCCTGGAAAAGCTGATGAGTTT | epegRNA with | spacer + scaffold + |
| GC3_CT1_ | | AAGAGCTAGAAATAGCAAGTTTAA | UA flip, 4CAG | flap + linker + 3′motif |
| CT2 | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGGAAGGATTTCAGCGATTCG | | |
| | | AAGGCTTTCATCAGCTTTTCCAATC | | |
| | | ACTCGCGGTTCTATCTAGTTACGC | | |
| | | GTTAAACCAACTAGAA | | |
| htt_h40_GC2_ | 746 | GACCCTGGAAAAGCTGATGAGTTT | epegRNA with | spacer + scaffold + |
| GC3_CT1_ | | AAGAGCTAGAAATAGCAAGTTTAA | UA flip, 4CAG | flap + linker + 3′motif |
| GA2 | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGGAAGGATTTCAGCGACTCA | | |
| | | AAGGCTTTCATCAGCTTTTCCAATC | | |
| | | ACTCGCGGTTCTATCTAGTTACGC | | |
| | | GTTAAACCAACTAGAA | | |
| htt_h40_GC2_ | 747 | GACCCTGGAAAAGCTGATGAGTTT | epegRNA with | spacer + scaffold + |
| GC3_GA1_ | | AAGAGCTAGAAATAGCAAGTTTAA | UA flip, 4CAG | flap + linker + 3′motif |
| CT2 | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGAAAGGACTTCAGCGATTCG | | |
| | | AAGGCTTTCATCAGCTTTTCCAATC | | |
| | | ACTCGCGGTTCTATCTAGTTACGC | | |
| | | GTTAAACCAACTAGAA | | |
| htt_h40_GC2_ | 748 | GACCCTGGAAAAGCTGATGAGTTT | epegRNA with | spacer + scaffold + |
| GC3_GA1_ | | AAGAGCTAGAAATAGCAAGTTTAA | UA flip, 4CAG | flap + linker + 3′motif |
| GA2 | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGAAAGGACTTCAGCGACTCA | | |
| | | AAGGCTTTCATCAGCTTTTCCAATC | | |
| | | ACTCGCGGTTCTATCTAGTTACGC | | |
| | | GTTAAACCAACTAGAA | | |
| htt_h40_GC2_ | 749 | GACCCTGGAAAAGCTGATGAGTTT | epegRNA with | spacer + scaffold + |
| GC3_CT2_ | | AAGAGCTAGAAATAGCAAGTTTAA | UA flip, 4CAG | flap + linker + 3′motif |
| GA2 | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGGAAGGACTTCAGCGATTCA | | |
| | | AAGGCTTTCATCAGCTTTTCCAATC | | |
| | | ACTCGCGGTTCTATCTAGTTACGC | | |
| | | GTTAAACCAACTAGAA | | |
| htt_h40_GC2_ | 750 | GACCCTGGAAAAGCTGATGAGTTT | epegRNA with | spacer + scaffold + |
| GC4_CT1_ | | AAGAGCTAGAAATAGCAAGTTTAA | UA flip, 4CAG | flap + linker + 3′motif |
| CT2 | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGGAAGGATTTCAGGGATTCG | | |
| | | AACGCTTTCATCAGCTTTTCCAATC | | |
| | | ACTCGCGGTTCTATCTAGTTACGC | | |
| | | GTTAAACCAACTAGAA | | |
| htt_h40_GC2_ | 751 | GACCCTGGAAAAGCTGATGAGTTT | epegRNA with | spacer + scaffold + |
| GC4_CT1_ | | AAGAGCTAGAAATAGCAAGTTTAA | UA flip, 4CAG | flap + linker + 3′motif |
| GA2 | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGGAAGGATTTCAGGGACTCA | | |
| | | AACGCTTTCATCAGCTTTTCCAATC | | |
| | | ACTCGCGGTTCTATCTAGTTACGC | | |
| | | GTTAAACCAACTAGAA | | |
| htt_h40_GC2_ | 752 | GACCCTGGAAAAGCTGATGAGTTT | epegRNA with | spacer + scaffold + |
| GC4_GA1_ | | AAGAGCTAGAAATAGCAAGTTTAA | UA flip, 4CAG | flap + linker + 3′motif |
| CT2 | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGAAAGGACTTCAGGGATTCG | | |
| | | AACGCTTTCATCAGCTTTTCCAATC | | |
| | | ACTCGCGGTTCTATCTAGTTACGC | | |
| | | GTTAAACCAACTAGAA | | |
| htt_h40_GC2_ | 753 | GACCCTGGAAAAGCTGATGAGTTT | epegRNA with | spacer + scaffold + |
| GC4_GA1_ | | AAGAGCTAGAAATAGCAAGTTTAA | UA flip, 4CAG | flap + linker + 3′motif |
| GA2 | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGAAAGGACTTCAGGGACTCA | | |
| | | AACGCTTTCATCAGCTTTTCCAATC | | |
| | | ACTCGCGGTTCTATCTAGTTACGC | | |
| | | GTTAAACCAACTAGAA | | |
| htt_h40_GC2_ | 752 | GACCCTGGAAAAGCTGATGAGTTT | epegRNA with | spacer + scaffold + |
| GC4_CT2_ | | AAGAGCTAGAAATAGCAAGTTTAA | UA flip, 4CAG | flap + linker + 3′motif |
| GA2 | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGAAAGGACTTCAGGGATTCG | | |
| | | AACGCTTTCATCAGCTTTTCCAATC | | |
| | | ACTCGCGGTTCTATCTAGTTACGC | | |
| | | GTTAAACCAACTAGAA | | |
| htt_h40_GC2_ | 754 | GACCCTGGAAAAGCTGATGAGTTT | epegRNA with | spacer + scaffold + |
| CT1_GA1_ | | AAGAGCTAGAAATAGCAAGTTTAA | UA flip, 4CAG | flap + linker + 3′motif |
| GA2 | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGAAAGGATTTCAGGGACTCA | | |
| | | AAGGCTTTCATCAGCTTTTCCAATC | | |
| | | ACTCGCGGTTCTATCTAGTTACGC | | |
| | | GTTAAACCAACTAGAA | | |
| htt_h40_GC2_ | 755 | GACCCTGGAAAAGCTGATGAGTTT | epegRNA with | spacer + scaffold + |
| CT1_CT2_ | | AAGAGCTAGAAATAGCAAGTTTAA | UA flip, 4CAG | flap + linker + 3′motif |
| GA2 | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGGAAGGATTTCAGGGATTCA | | |
| | | AAGGCTTTCATCAGCTTTTCCAATC | | |
| | | ACTCGCGGTTCTATCTAGTTACGC | | |
| | | GTTAAACCAACTAGAA | | |
| htt_h40_GC3_ | 756 | GACCCTGGAAAAGCTGATGAGTTT | epegRNA with | spacer + scaffold + |
| GC4_GA1_ | | AAGAGCTAGAAATAGCAAGTTTAA | UA flip, 4CAG | flap + linker + 3′motif |
| CT2 | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGAAAGGACTTGAGCGATTCG | | |
| | | AACGCTTTCATCAGCTTTTCCAATC | | |
| | | ACTCGCGGTTCTATCTAGTTACGC | | |
| | | GTTAAACCAACTAGAA | | |
| htt_h40_GC3_ | 757 | GACCCTGGAAAAGCTGATGAGTTT | epegRNA with | spacer + scaffold + |
| GC4_GA1_ | | AAGAGCTAGAAATAGCAAGTTTAA | UA flip, 4CAG | flap + linker + 3′motif |
| GA2 | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGAAAGGACTTGAGCGACTCA | | |
| | | AACGCTTTCATCAGCTTTTCCAATC | | |
| | | ACTCGCGGTTCTATCTAGTTACGC | | |
| | | GTTAAACCAACTAGAA | | |
| htt_h40_GC3_ | 758 | GACCCTGGAAAAGCTGATGAGTTT | epegRNA with | spacer + scaffold + |
| GC4_CT2_ | | AAGAGCTAGAAATAGCAAGTTTAA | UA flip, 4CAG | flap + linker + 3′motif |
| GA2 | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGGAAGGACTTGAGCGATTCA | | |
| | | AACGCTTTCATCAGCTTTTCCAATC | | |
| | | ACTCGCGGTTCTATCTAGTTACGC | | |
| | | GTTAAACCAACTAGAA | | |
| htt_h40_GC3_ | 759 | GACCCTGGAAAAGCTGATGAGTTT | epegRNA with | spacer + scaffold + |
| CT1_GA1_ | | AAGAGCTAGAAATAGCAAGTTTAA | UA flip, 4CAG | flap + linker + 3′motif |
| GA2 | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGAAAGGATTTGAGCGACTCA | | |
| | | AAGGCTTTCATCAGCTTTTCCAATC | | |
| | | ACTCGCGGTTCTATCTAGTTACGC | | |
| | | GTTAAACCAACTAGAA | | |
| htt_h40_GC3_ | 760 | GACCCTGGAAAAGCTGATGAGTTT | epegRNA with | spacer + scaffold + |
| CT1_CT2_ | | AAGAGCTAGAAATAGCAAGTTTAA | UA flip, 4CAG | flap + linker + 3′motif |
| GA2 | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGGAAGGATTTGAGCGATTCA | | |
| | | AAGGCTTTCATCAGCTTTTCCAATC | | |
| | | ACTCGCGGTTCTATCTAGTTACGC | | |
| | | GTTAAACCAACTAGAA | | |
| htt_h40_GC4_ | 761 | GACCCTGGAAAAGCTGATGAGTTT | epegRNA with | spacer + scaffold + |
| CT1_CT2_ | | AAGAGCTAGAAATAGCAAGTTTAA | UA flip, 4CAG | flap + linker + 3′motif |
| GA2 | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGGAAGGATTTGAGGGATTCA | | |
| | | AACGCTTTCATCAGCTTTTCCAATC | | |
| | | ACTCGCGGTTCTATCTAGTTACGC | | |
| | | GTTAAACCAACTAGAA | | |
| htt_h40_h40_ | 762 | GACCCTGGAAAAGCTGATGAGTTT | epegRNA with | spacer + scaffold + |
| GC2_GC3_ | | AAGAGCTAGAAATAGCAAGTTTAA | UA flip, 4CAG | flap + linker + 3′motif |
| GC4_CT2_ | | ATAAGGCTAGTCCGTTATCAACTT | insertion (6Q) | |
| GA1 | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGAAAGGACTTCAGCGATTCG | | |
| | | AACGCTTTCATCAGCTTTTCCAATC | | |
| | | ACTCGCGGTTCTATCTAGTTACGC | | |
| | | GTTAAACCAACTAGAA | | |
| htt_h40_GC2_ | 763 | GACCCTGGAAAAGCTGATGAGTTT | epegRNA with | spacer + scaffold + |
| GC3_GC4_ | | AAGAGCTAGAAATAGCAAGTTTAA | UA flip, 4CAG | flap + linker + 3′motif |
| CT1_GA1 | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGAAAGGATTTCAGCGACTCG | | |
| | | AACGCTTTCATCAGCTTTTCCAATC | | |
| | | ACTCGCGGTTCTATCTAGTTACGC | | |
| | | GTTAAACCAACTAGAA | | |
| htt_h40_GC2 | 764 | GACCCTGGAAAAGCTGATGAGTTT | epegRNA with | spacer + scaffold + |
| GC3_GC4 | | AAGAGCTAGAAATAGCAAGTTTAA | UA flip, 4CAG | flap + linker + 3′motif |
| CT1_CT2 | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGGAAGGATTTCAGCGATTCG | | |
| | | AACGCTTTCATCAGCTTTTCCAATC | | |
| | | ACTCGCGGTTCTATCTAGTTACGC | | |
| | | GTTAAACCAACTAGAA | | |
| htt_h40_GC2 | 765 | GACCCTGGAAAAGCTGATGAGTTT | epegRNA with | spacer + scaffold + |
| GC3_GC4 | | AAGAGCTAGAAATAGCAAGTTTAA | UA flip, 4CAG | flap + linker + 3′motif |
| CT1_GA2 | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGGAAGGATTTCAGCGACTCA | | |
| | | AACGCTTTCATCAGCTTTTCCAATC | | |
| | | ACTCGCGGTTCTATCTAGTTACGC | | |
| | | GTTAAACCAACTAGAA | | |
| htt_h40_GC2_ | 766 | GACCCTGGAAAAGCTGATGAGTTT | epegRNA with | spacer + scaffold + |
| GC4_CT1_ | | AAGAGCTAGAAATAGCAAGTTTAA | UA flip, 4CAG | flap + linker + 3′motif |
| GA1_CT2 | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGAAAGGATTTCAGGGATTCG | | |
| | | AACGCTTTCATCAGCTTTTCCAATC | | |
| | | ACTCGCGGTTCTATCTAGTTACGC | | |
| | | GTTAAACCAACTAGAA | | |
| htt_h40_GC2_ | 767 | GACCCTGGAAAAGCTGATGAGTTT | epegRNA with | spacer + scaffold + |
| GC4_CT1_ | | AAGAGCTAGAAATAGCAAGTTTAA | UA flip, 4CAG | flap + linker + 3′motif |
| GA1_GA2 | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGAAAGGATTTCAGGGACTCA | | |
| | | AACGCTTTCATCAGCTTTTCCAATC | | |
| | | ACTCGCGGTTCTATCTAGTTACGC | | |
| | | GTTAAACCAACTAGAA | | |
| htt_h40_GC2_ | 768 | GACCCTGGAAAAGCTGATGAGTTT | epegRNA with | spacer + scaffold + |
| CT1_GA1_ | | AAGAGCTAGAAATAGCAAGTTTAA | UA flip, 4CAG | flap + linker + 3′motif |
| CT2_GA2 | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGAAAGGATTTCAGGGATTCA | | |
| | | AAGGCTTTCATCAGCTTTTCCAATC | | |
| | | ACTCGCGGTTCTATCTAGTTACGC | | |
| | | GTTAAACCAACTAGAA | | |
| htt_h40_GC2_ | 769 | GACCCTGGAAAAGCTGATGAGTTT | epegRNA with | spacer + scaffold + |
| CT1_GA1_ | | AAGAGCTAGAAATAGCAAGTTTAA | UA flip, 4CAG | flap + linker + 3′motif |
| CT2_GC3 | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGAAAGGATTTCAGCGATTCG | | |
| | | AAGGCTTTCATCAGCTTTTCCAATC | | |
| | | ACTCGCGGTTCTATCTAGTTACGC | | |
| | | GTTAAACCAACTAGAA | | |
| htt_h40_GC2_ | 766 | GACCCTGGAAAAGCTGATGAGTTT | epegRNA with | spacer + scaffold + |
| CT1_GA1_ | | AAGAGCTAGAAATAGCAAGTTTAA | UA flip, 4CAG | flap + linker + 3′motif |
| CT2_GC4 | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGAAAGGATTTCAGGGATTCG | | |
| | | AACGCTTTCATCAGCTTTTCCAATC | | |
| | | ACTCGCGGTTCTATCTAGTTACGC | | |
| | | GTTAAACCAACTAGAA | | |
| htt_h40_GC2_ | 770 | GACCCTGGAAAAGCTGATGAGTTT | epegRNA with | spacer + scaffold + |
| GA1_CT2_ | | AAGAGCTAGAAATAGCAAGTTTAA | UA flip, 4CAG | flap + linker + 3′motif |
| GA2_GC3 | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGAAAGGACTTCAGCGATTCA | | |
| | | AAGGCTTTCATCAGCTTTTCCAATC | | |
| | | ACTCGCGGTTCTATCTAGTTACGC | | |
| | | GTTAAACCAACTAGAA | | |
| htt_h40_GC2_ | 771 | GACCCTGGAAAAGCTGATGAGTTT | epegRNA with | spacer + scaffold + |
| GA1_CT2_ | | AAGAGCTAGAAATAGCAAGTTTAA | UA flip, 4CAG | flap + linker + 3′motif |
| GA2_GC4 | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGAAAGGACTTCAGGGATTCA | | |
| | | AACGCTTTCATCAGCTTTTCCAATC | | |
| | | ACTCGCGGTTCTATCTAGTTACGC | | |
| | | GTTAAACCAACTAGAA | | |
| htt_h40_GC2_ | 772 | GACCCTGGAAAAGCTGATGAGTTT | epegRNA with | spacer + scaffold + |
| GC3_GC4_ | | AAGAGCTAGAAATAGCAAGTTTAA | UA flip, 4CAG | flap + linker + 3′motif |
| GA1_GA2 | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGAAAGGACTTCAGCGACTCA | | |
| | | AACGCTTTCATCAGCTTTTCCAATC | | |
| | | ACTCGCGGTTCTATCTAGTTACGC | | |
| | | GTTAAACCAACTAGAA | | |
| htt_h40_GC2_ | 773 | GACCCTGGAAAAGCTGATGAGTTT | epegRNA with | spacer + scaffold + |
| GC3_GC4_ | | AAGAGCTAGAAATAGCAAGTTTAA | UA flip, 4CAG | flap + linker + 3′motif |
| CT2_GA2 | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGGAAGGACTTCAGCGATTCA | | |
| | | AACGCTTTCATCAGCTTTTCCAATC | | |
| | | ACTCGCGGTTCTATCTAGTTACGC | | |
| | | GTTAAACCAACTAGAA | | |
| htt_h40_GC3_ | 774 | GACCCTGGAAAAGCTGATGAGTTT | epegRNA with | spacer + scaffold + |
| GC4_CT1_ | | AAGAGCTAGAAATAGCAAGTTTAA | UA flip, 4CAG | flap + linker + 3′motif |
| GA1_GA2 | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGAAAGGATTTGAGCGACTCA | | |
| | | AACGCTTTCATCAGCTTTTCCAATC | | |
| | | ACTCGCGGTTCTATCTAGTTACGC | | |
| | | GTTAAACCAACTAGAA | | |
| htt_h40_GC3_ | 775 | GACCCTGGAAAAGCTGATGAGTTT | epegRNA with | spacer + scaffold + |
| GC4_GA1_ | | AAGAGCTAGAAATAGCAAGTTTAA | UA flip, 4CAG | flap + linker + 3′motif |
| CT2_GA2 | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGAAAGGACTTGAGTGACTCA | | |
| | | AACGCTTTCATCAGCTTTTCCAATC | | |
| | | ACTCGCGGTTCTATCTAGTTACGC | | |
| | | GTTAAACCAACTAGAA | | |
| htt_h40_GC4_ | 776 | GACCCTGGAAAAGCTGATGAGTTT | epegRNA with | spacer + scaffold + |
| CT1_GA1_ | | AAGAGCTAGAAATAGCAAGTTTAA | UA flip, 4CAG | flap + linker + 3′motif |
| CT2_GA2 | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGAAAGGATTTGAGGGATTCA | | |
| | | AACGCTTTCATCAGCTTTTCCAATC | | |
| | | ACTCGCGGTTCTATCTAGTTACGC | | |
| | | GTTAAACCAACTAGAA | | |
| htt_h40_GC4_ | 766 | GACCCTGGAAAAGCTGATGAGTTT | epegRNA with | spacer + scaffold + |
| CT1_GA1 | | AAGAGCTAGAAATAGCAAGTTTAA | UA flip, 4CAG | flap + linker + 3′motif |
| CT2_GC2 | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGAAAGGATTTCAGGGATTCG | | |
| | | AACGCTTTCATCAGCTTTTCCAATC | | |
| | | ACTCGCGGTTCTATCTAGTTACGC | | |
| | | GTTAAACCAACTAGAA | | |
| htt_h40_GC4_ | 777 | GACCCTGGAAAAGCTGATGAGTTT | epegRNA with | spacer + scaffold + |
| CT1_GA1_ | | AAGAGCTAGAAATAGCAAGTTTAA | UA flip, 4CAG | flap + linker + 3′motif |
| CT2_GC3 | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGAAAGGATTTGAGCGATTCG | | |
| | | AACGCTTTCATCAGCTTTTCCAATC | | |
| | | ACTCGCGGTTCTATCTAGTTACGC | | |
| | | GTTAAACCAACTAGAA | | |
| htt_h40_CT1_ | 778 | GACCCTGGAAAAGCTGATGAGTTT | epegRNA with | spacer + scaffold + |
| GA1_CT2_ | | AAGAGCTAGAAATAGCAAGTTTAA | UA flip, 4CAG | flap + linker + 3′motif |
| GA2_GC3 | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGAAAGGATTTGAGCGATTCA | | |
| | | AAGGCTTTCATCAGCTTTTCCAATC | | |
| | | ACTCGCGGTTCTATCTAGTTACGC | | |
| | | GTTAAACCAACTAGAA | | |
| htt_h40_GC2_ | 779 | GACCCTGGAAAAGCTGATGAGTTT | epegRNA with | spacer + scaffold + |
| GC4_CT1_ | | AAGAGCTAGAAATAGCAAGTTTAA | UA flip, 4CAG | flap + linker + 3′motif |
| CT2_GA2 | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGGAAGGATTTCAGGGATTCA | | |
| | | AACGCTTTCATCAGCTTTTCCAATC | | |
| | | ACTCGCGGTTCTATCTAGTTACGC | | |
| | | GTTAAACCAACTAGAA | | |
| htt_h40_GC3_ | 774 | GACCCTGGAAAAGCTGATGAGTTT | epegRNA with | spacer + scaffold + |
| GC4_CT1_ | | AAGAGCTAGAAATAGCAAGTTTAA | UA flip, 4CAG | flap + linker + 3′motif |
| GA1_GA2 | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGAAAGGATTTGAGCGACTCA | | |
| | | AACGCTTTCATCAGCTTTTCCAATC | | |
| | | ACTCGCGGTTCTATCTAGTTACGC | | |
| | | GTTAAACCAACTAGAA | | |
| htt_h40_GC3_ | 780 | GACCCTGGAAAAGCTGATGAGTTT | epegRNA with | spacer + scaffold + |
| GC4_CT1_ | | AAGAGCTAGAAATAGCAAGTTTAA | UA flip, 4CAG | flap + linker + 3′motif |
| CT2_GA2 | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGGAAGGATTTGAGCGATTCA | | |
| | | AACGCTTTCATCAGCTTTTCCAATC | | |
| | | ACTCGCGGTTCTATCTAGTTACGC | | |
| | | GTTAAACCAACTAGAA | | |
| htt_h40_GC2_ | 781 | GACCCTGGAAAAGCTGATGAGTTT | epegRNA with | spacer + scaffold + |
| GC3_GC4_ | | AAGAGCTAGAAATAGCAAGTTTAA | UA flip, 4CAG | flap + linker + 3′motif |
| CT1_GA1_ | | ATAAGGCTAGTCCGTTATCAACTT | insertion (6Q) | |
| CT2 | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGAAAGGATTTCAGCGATTCG | | |
| | | AACGCTTTCATCAGCTTTTCCAATC | | |
| | | ACTCGCGGTTCTATCTAGTTACGC | | |
| | | GTTAAACCAACTAGAA | | |
| htt_h40_GC2_ | 782 | GACCCTGGAAAAGCTGATGAGTTT | epegRNA with | spacer + scaffold + |
| GC3_GC4_ | | AAGAGCTAGAAATAGCAAGTTTAA | UA flip, 4CAG | flap + linker + 3′motif |
| CT1_GA1_ | | ATAAGGCTAGTCCGTTATCAACTT | insertion (6Q) | |
| GA2 | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGAAAGGATTTCAGCGACTCA | | |
| | | AACGCTTTCATCAGCTTTTCCAATC | | |
| | | ACTCGCGGTTCTATCTAGTTACGC | | |
| | | GTTAAACCAACTAGAA | | |
| htt_h40_GC3_ | 783 | GACCCTGGAAAAGCTGATGAGTTT | epegRNA with | spacer + scaffold + |
| GC4_CT1_ | | AAGAGCTAGAAATAGCAAGTTTAA | UA flip, 4CAG | flap + linker + 3′motif |
| GA1_CT2_ | | ATAAGGCTAGTCCGTTATCAACTT | insertion (6Q) | |
| GA2 | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGAAAGGATTTGAGTGACTCA | | |
| | | AACGCTTTCATCAGCTTTTCCAATC | | |
| | | ACTCGCGGTTCTATCTAGTTACGC | | |
| | | GTTAAACCAACTAGAA | | |
| htt_h40_GC4_ | 784 | GACCCTGGAAAAGCTGATGAGTTT | epegRNA with | spacer + scaffold + |
| CT1_GA1_ | | AAGAGCTAGAAATAGCAAGTTTAA | UA flip, 4CAG | flap + linker + 3′motif |
| CT2_GA2_ | | ATAAGGCTAGTCCGTTATCAACTT | insertion (6Q) | |
| GC2 | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGAAAGGATTTCAGGGATTCA | | |
| | | AACGCTTTCATCAGCTTTTCCAATC | | |
| | | ACTCGCGGTTCTATCTAGTTACGC | | |
| | | GTTAAACCAACTAGAA | | |
| htt_h40_CT1_ | 785 | GACCCTGGAAAAGCTGATGAGTTT | epegRNA with | spacer + scaffold + |
| GA1_CT2_ | | AAGAGCTAGAAATAGCAAGTTTAA | UA flip, 4CAG | flap + linker + 3′motif |
| GA2_GC2_ | | ATAAGGCTAGTCCGTTATCAACTT | insertion (6Q) | |
| GC3 | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGAAAGGATTTCAGCGATTCA | | |
| | | AAGGCTTTCATCAGCTTTTCCAATC | | |
| | | ACTCGCGGTTCTATCTAGTTACGC | | |
| | | GTTAAACCAACTAGAA | | |
| htt_h40_GA1_ | 784 | GACCCTGGAAAAGCTGATGAGTTT | epegRNA with | spacer + scaffold + |
| CT2_GA2_ | | AAGAGCTAGAAATAGCAAGTTTAA | UA flip, 4CAG | flap + linker + 3′motif |
| GC2_GC4_ | | ATAAGGCTAGTCCGTTATCAACTT | insertion (6Q) | |
| CT1 | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGAAAGGATTTCAGGGATTCA | | |
| | | AACGCTTTCATCAGCTTTTCCAATC | | |
| | | ACTCGCGGTTCTATCTAGTTACGC | | |
| | | GTTAAACCAACTAGAA | | |
| htt_h40_GC2_ | 786 | GACCCTGGAAAAGCTGATGAGTTT | epegRNA with | spacer + scaffold + |
| GC3_GC4_ | | AAGAGCTAGAAATAGCAAGTTTAA | UA flip, 4CAG | flap + linker + 3′motif |
| CT1_CT2_ | | ATAAGGCTAGTCCGTTATCAACTT | insertion (6Q) | |
| GA2 | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGGAAGGATTTCAGCGATTCA | | |
| | | AACGCTTTCATCAGCTTTTCCAATC | | |
| | | ACTCGCGGTTCTATCTAGTTACGC | | |
| | | GTTAAACCAACTAGAA | | |
| htt_h40_GC2_ | 787 | GACCCTGGAAAAGCTGATGAGTTT | epegRNA with | spacer + scaffold + |
| GC3_GC4_ | | AAGAGCTAGAAATAGCAAGTTTAA | UA flip, 4CAG | flap + linker + 3′motif |
| CT1_GA1_ | | ATAAGGCTAGTCCGTTATCAACTT | insertion (6Q) | |
| CT2_GA2 | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGAAAGGATTTCAGCGATTCA | | |
| | | AACGCTTTCATCAGCTTTTCCAATC | | |
| | | ACTCGCGGTTCTATCTAGTTACGC | | |
| | | GTTAAACCAACTAGAA | | |
| htt_h40_ | 788 | GACCCTGGAAAAGCTGATGAGTTT | epegRNA with | spacer + scaffold + |
| recoded | | AAGAGCTAGAAATAGCAAGTTTAA | UA flip, 4CAG | flap + linker + 3′motif |
| | | ATAAGGCTAGTCCGTTATCAACTT | insertion (6Q) | |
| | | GAAAAAGTGGCACCGAGTCGGTGC | | |
| | | GAGGAGGCGGCGGCGGCGGCGGC | | |
| | | GGCGGTGGCGGCTGTTGCTGCTGC | | |
| | | TGCTGAAACGATTTCAGCGATTCA | | |
| | | AACGCTTTCATCAGCTTTTCCAATC | | |
| | | ACTCGCGGTTCTATCTAGTTACGC | | |
| | | GTTAAACCAACTAGAA | | |
| htt_h31_GC2_ | 789 | GACCCTGGAAAAGCTGATGAGTTT | epegRNA for | spacer + scaffold + |
| 9CAG | | AAGAGCTAGAAATAGCAAGTTTAA | various CAG | flap + linker + 3′motif |
| | | ATAAGGCTAGTCCGTTATCAACTT | replacement, | |
| | | GAAAAAGTGGCACCGAGTCGGTGC | with UA flip | |
| | | GCGGCGGCGGCGGCGGCGGTGGC | | |
| | | GGCTGTTGCTGCTGCTGCTGCTGCT | | |
| | | GCTGCTGCTGGAAGGACTTCAGGG | | |
| | | ACTCGAAGGCTTTCATCAGCTTTTC | | |
| | | CAATCACTCGCGGTTCTATCTAGTT | | |
| | | ACGCGTTAAACCAACTAGAA | | |
| htt_h31_CT2_ | 790 | GACCCTGGAAAAGCTGATGAGTTT | epegRNA for | spacer + scaffold + |
| 9CAG | | AAGAGCTAGAAATAGCAAGTTTAA | various CAG | flap + linker + 3′motif |
| | | ATAAGGCTAGTCCGTTATCAACTT | replacement, | |
| | | GAAAAAGTGGCACCGAGTCGGTGC | with UA flip | |
| | | GCGGCGGCGGCGGCGGCGGTGGC | | |
| | | GGCTGTTGCTGCTGCTGCTGCTGCT | | |
| | | GCTGCTGCTGGAAGGACTTGAGGG | | |
| | | ATTCGAAGGCTTTCATCAGCTTTTC | | |
| | | CAATCACTCGCGGTTCTATCTAGTT | | |
| | | ACGCGTTAAACCAACTAGAA | | |
| htt_h31_ | 791 | GACCCTGGAAAAGCTGATGAGTTT | epegRNA for | spacer + scaffold + |
| 9CAR9 | | AAGAGCTAGAAATAGCAAGTTTAA | various CAG | flap + linker + 3′motif |
| | | ATAAGGCTAGTCCGTTATCAACTT | replacement, | |
| | | GAAAAAGTGGCACCGAGTCGGTGC | with UA flip | |
| | | GCGGCGGCGGCGGCGGCGGTGGC | | |
| | | GGCTGTTGTTGTTGTTGTTGTTGTT | | |
| | | GTTGTTGTTGGAAGGACTTGAGGG | | |
| | | ACTCGAAGGCTTTCATCAGCTTTTC | | |
| | | CAATCACTCGCGGTTCTATCTAGTT | | |
| | | ACGCGTTAAACCAACTAGAA | | |
| htt_h31_ | 792 | GACCCTGGAAAAGCTGATGAGTTT | epegRNA for | spacer + scaffold + |
| 9CAR4 | | AAGAGCTAGAAATAGCAAGTTTAA | various CAG | flap + linker + 3′motif |
| | | ATAAGGCTAGTCCGTTATCAACTT | replacement, | |
| | | GAAAAAGTGGCACCGAGTCGGTGC | with UA flip | |
| | | GCGGCGGCGGCGGCGGCGGTGGC | | |
| | | GGCTGTTGCTGCTGTTGCTGTTGCT | | |
| | | GTTGCTGTTGGAAGGACTTGAGGG | | |
| | | ACTCGAAGGCTTTCATCAGCTTTTC | | |
| | | CAATCACTCGCGGTTCTATCTAGTT | | |
| | | ACGCGTTAAACCAACTAGAA | | |
| htt_h31_GC2_ | 793 | GACCCTGGAAAAGCTGATGAGTTT | epegRNA for | spacer + scaffold + |
| 9CAR5 | | AAGAGCTAGAAATAGCAAGTTTAA | various CAG | flap + linker + 3′motif |
| | | ATAAGGCTAGTCCGTTATCAACTT | replacement, | |
| | | GAAAAAGTGGCACCGAGTCGGTGC | with UA flip | |
| | | GCGGCGGCGGCGGCGGCGGTGGC | | |
| | | GGCTGTTGTTGCTGTTGCTGTTGCT | | |
| | | GTTGCTGTTGGAAGGACTTCAGGG | | |
| | | ACTCGAAGGCTTTCATCAGCTTTTC | | |
| | | CAATCACTCGCGGTTCTATCTAGTT | | |
| | | ACGCGTTAAACCAACTAGAA | | |
| htt_h31_CT2_ | 794 | GACCCTGGAAAAGCTGATGAGTTT | epegRNA for | spacer + scaffold + |
| 9CAR5 | | AAGAGCTAGAAATAGCAAGTTTAA | various CAG | flap + linker + 3′motif |
| | | ATAAGGCTAGTCCGTTATCAACTT | replacement, | |
| | | GAAAAAGTGGCACCGAGTCGGTGC | with UA flip | |
| | | GCGGCGGCGGCGGCGGCGGTGGC | | |
| | | GGCTGTTGTTGCTGTTGCTGTTGCT | | |
| | | GTTGCTGTTGGAAGGACTTGAGGG | | |
| | | ATTCGAAGGCTTTCATCAGCTTTTC | | |
| | | CAATCACTCGCGGTTCTATCTAGTT | | |
| | | ACGCGTTAAACCAACTAGAA | | |
| htt_h31_GC2_ | 795 | GACCCTGGAAAAGCTGATGAGTTT | epegRNA for | spacer + scaffold + |
| 9CAR9 | | AAGAGCTAGAAATAGCAAGTTTAA | various CAG | flap + linker + 3′motif |
| | | ATAAGGCTAGTCCGTTATCAACTT | replacement, | |
| | | GAAAAAGTGGCACCGAGTCGGTGC | with UA flip | |
| | | GCGGCGGCGGCGGCGGCGGTGGC | | |
| | | GGCTGTTGTTGTTGTTGTTGTTGTT | | |
| | | GTTGTTGTTGGAAGGACTTCAGGG | | |
| | | ACTCGAAGGCTTTCATCAGCTTTTC | | |
| | | CAATCACTCGCGGTTCTATCTAGTT | | |
| | | ACGCGTTAAACCAACTAGAA | | |
| htt_h31_CT2_ | 796 | GACCCTGGAAAAGCTGATGAGTTT | epegRNA for | spacer + scaffold + |
| 9CAR9 | | AAGAGCTAGAAATAGCAAGTTTAA | various CAG | flap + linker + 3′motif |
| | | ATAAGGCTAGTCCGTTATCAACTT | replacement, | |
| | | GAAAAAGTGGCACCGAGTCGGTGC | with UA flip | |
| | | GCGGCGGCGGCGGCGGCGGTGGC | | |
| | | GGCTGTTGTTGTTGTTGTTGTTGTT | | |
| | | GTTGTTGTTGGAAGGACTTGAGGG | | |
| | | ATTCGAAGGCTTTCATCAGCTTTTC | | |
| | | CAATCACTCGCGGTTCTATCTAGTT | | |
| | | ACGCGTTAAACCAACTAGAA | | |
| htt_h31_CT2_ | 797 | GACCCTGGAAAAGCTGATGAGTTT | epegRNA for | spacer + scaffold + |
| GC2_9CAR9 | | AAGAGCTAGAAATAGCAAGTTTAA | various CAG | flap + linker + 3′motif |
| | | ATAAGGCTAGTCCGTTATCAACTT | replacement, | |
| | | GAAAAAGTGGCACCGAGTCGGTGC | with UA flip | |
| | | GCGGCGGCGGCGGCGGCGGTGGC | | |
| | | GGCTGTTGTTGTTGTTGTTGTTGTT | | |
| | | GTTGTTGTTGGAAGGACTTCAGGG | | |
| | | ATTCGAAGGCTTTCATCAGCTTTTC | | |
| | | CAATCACTCGCGGTTCTATCTAGTT | | |
| | | ACGCGTTAAACCAACTAGAA | | |
| htt_h31_GC2_ | 798 | GACCCTGGAAAAGCTGATGAGTTT | epegRNA for | spacer + scaffold + |
| 9CAR4 | | AAGAGCTAGAAATAGCAAGTTTAA | various CAG | flap + linker + 3′motif |
| | | ATAAGGCTAGTCCGTTATCAACTT | replacement, | |
| | | GAAAAAGTGGCACCGAGTCGGTGC | with UA flip | |
| | | GCGGCGGCGGCGGCGGCGGTGGC | | |
| | | GGCTGTTGCTGCTGTTGCTGTTGCT | | |
| | | GTTGCTGTTGGAAGGACTTCAGGG | | |
| | | ACTCGAAGGCTTTCATCAGCTTTTC | | |
| | | CAATCACTCGCGGTTCTATCTAGTT | | |
| | | ACGCGTTAAACCAACTAGAA | | |
| htt_h31_CT2_ | 799 | GACCCTGGAAAAGCTGATGAGTTT | epegRNA for | spacer + scaffold + |
| 9CAR4 | | AAGAGCTAGAAATAGCAAGTTTAA | various CAG | flap + linker + 3′motif |
| | | ATAAGGCTAGTCCGTTATCAACTT | replacement, | |
| | | GAAAAAGTGGCACCGAGTCGGTGC | with UA flip | |
| | | GCGGCGGCGGCGGCGGCGGTGGC | | |
| | | GGCTGTTGCTGCTGTTGCTGTTGCT | | |
| | | GTTGCTGTTGGAAGGACTTGAGGG | | |
| | | ATTCGAAGGCTTTCATCAGCTTTTC | | |
| | | CAATCACTCGCGGTTCTATCTAGTT | | |
| | | ACGCGTTAAACCAACTAGAA | | |
| htt_h31_CT2_ | 800 | GACCCTGGAAAAGCTGATGAGTTT | epegRNA for | spacer + scaffold + |
| GC2_9CAR4 | | AAGAGCTAGAAATAGCAAGTTTAA | various CAG | flap + linker + 3′motif |
| | | ATAAGGCTAGTCCGTTATCAACTT | replacement, | |
| | | GAAAAAGTGGCACCGAGTCGGTGC | with UA flip | |
| | | GCGGCGGCGGCGGCGGCGGTGGC | | |
| | | GGCTGTTGCTGCTGTTGCTGTTGCT | | |
| | | GTTGCTGTTGGAAGGACTTCAGGG | | |
| | | ATTCGAAGGCTTTCATCAGCTTTTC | | |
| | | CAATCACTCGCGGTTCTATCTAGTT | | |
| | | ACGCGTTAAACCAACTAGAA | | |
| htt_h40_ | 801 | GACCCTGGAAAAGCTGATGAGTTT | epegRNA for | spacer + scaffold + |
| 9CAR9 | | AAGAGCTAGAAATAGCAAGTTTAA | various CAG | flap + linker + 3′motif |
| | | ATAAGGCTAGTCCGTTATCAACTT | replacement, | |
| | | GAAAAAGTGGCACCGAGTCGGTGC | with UA flip | |
| | | GAGGAGGCGGCGGCGGCGGCGGC | | |
| | | GGCGGTGGCGGCTGTTGTTGTTGT | | |
| | | TGTTGTTGTTGTTGTTGTTGGAAGG | | |
| | | ACTTGAGGGACTCGAAGGCTTTCA | | |
| | | TCAGCTTTTCCAATCACTCGCGGTT | | |
| | | CTATCTAGTTACGCGTTAAACCAA | | |
| | | CTAGAA | | |
| htt_h40_ | 802 | GACCCTGGAAAAGCTGATGAGTTT | epegRNA for | spacer + scaffold + |
| 9CAR4 | | AAGAGCTAGAAATAGCAAGTTTAA | various CAG | flap + linker + 3′motif |
| | | ATAAGGCTAGTCCGTTATCAACTT | replacement, | |
| | | GAAAAAGTGGCACCGAGTCGGTGC | with UA flip | |
| | | GAGGAGGCGGCGGCGGCGGCGGC | | |
| | | GGCGGTGGCGGCTGTTGCTGCTGT | | |
| | | TGCTGTTGCTGTTGCTGTTGGAAG | | |
| | | GACTTGAGGGACTCGAAGGCTTTC | | |
| | | ATCAGCTTTTCCAATCACTCGCGGT | | |
| | | TCTATCTAGTTACGCGTTAAACCA | | |
| | | ACTAGAA | | |
| htt_h40_GC2_ | 803 | GACCCTGGAAAAGCTGATGAGTTT | epegRNA for | spacer + scaffold + |
| 9CAR5 | | AAGAGCTAGAAATAGCAAGTTTAA | various CAG | flap + linker + 3′motif |
| | | ATAAGGCTAGTCCGTTATCAACTT | replacement, | |
| | | GAAAAAGTGGCACCGAGTCGGTGC | with UA flip | |
| | | GAGGAGGCGGCGGCGGCGGCGGC | | |
| | | GGCGGTGGCGGCTGTTGTTGCTGT | | |
| | | TGCTGTTGCTGTTGCTGTTGGAAG | | |
| | | GACTTCAGGGACTCGAAGGCTTTC | | |
| | | ATCAGCTTTTCCAATCACTCGCGGT | | |
| | | TCTATCTAGTTACGCGTTAAACCA | | |
| | | ACTAGAA | | |
| htt_h40_CT2_ | 804 | GACCCTGGAAAAGCTGATGAGTTT | epegRNA for | spacer + scaffold + |
| 9CAR5 | | AAGAGCTAGAAATAGCAAGTTTAA | various CAG | flap + linker + 3′motif |
| | | ATAAGGCTAGTCCGTTATCAACTT | replacement, | |
| | | GAAAAAGTGGCACCGAGTCGGTGC | with UA flip | |
| | | GAGGAGGCGGCGGCGGCGGCGGC | | |
| | | GGCGGTGGCGGCTGTTGTTGCTGT | | |
| | | TGCTGTTGCTGTTGCTGTTGGAAG | | |
| | | GACTTGAGGGATTCGAAGGCTTTC | | |
| | | ATCAGCTTTTCCAATCACTCGCGGT | | |
| | | TCTATCTAGTTACGCGTTAAACCA | | |
| | | ACTAGAA | | |
| htt_h40_GC2_ | 805 | GACCCTGGAAAAGCTGATGAGTTT | epegRNA for | spacer + scaffold + |
| 9CAR9 | | AAGAGCTAGAAATAGCAAGTTTAA | various CAG | flap + linker + 3′motif |
| | | ATAAGGCTAGTCCGTTATCAACTT | replacement, | |
| | | GAAAAAGTGGCACCGAGTCGGTGC | with UA flip | |
| | | GAGGAGGCGGCGGCGGCGGCGGC | | |
| | | GGCGGTGGCGGCTGTTGTTGTTGT | | |
| | | TGTTGTTGTTGTTGTTGTTGGAAGG | | |
| | | ACTTCAGGGACTCGAAGGCTTTCA | | |
| | | TCAGCTTTTCCAATCACTCGCGGTT | | |
| | | CTATCTAGTTACGCGTTAAACCAA | | |
| | | CTAGAA | | |
| htt_h40_CT2_ | 806 | GACCCTGGAAAAGCTGATGAGTTT | epegRNA for | spacer + scaffold + |
| 9CAR9 | | AAGAGCTAGAAATAGCAAGTTTAA | various CAG | flap + linker + 3′motif |
| | | ATAAGGCTAGTCCGTTATCAACTT | replacement, | |
| | | GAAAAAGTGGCACCGAGTCGGTGC | with UA flip | |
| | | GAGGAGGCGGCGGCGGCGGCGGC | | |
| | | GGCGGTGGCGGCTGTTGTTGTTGT | | |
| | | TGTTGTTGTTGTTGTTGTTGGAAGG | | |
| | | ACTTGAGGGATTCGAAGGCTTTCA | | |
| | | TCAGCTTTTCCAATCACTCGCGGTT | | |
| | | CTATCTAGTTACGCGTTAAACCAA | | |
| | | CTAGAA | | |
| htt_h40_CT2_ | 807 | GACCCTGGAAAAGCTGATGAGTTT | epegRNA for | spacer + scaffold + |
| GC2_9CAR9 | | AAGAGCTAGAAATAGCAAGTTTAA | various CAG | flap + linker + 3′motif |
| | | ATAAGGCTAGTCCGTTATCAACTT | replacement, | |
| | | GAAAAAGTGGCACCGAGTCGGTGC | with UA flip | |
| | | GAGGAGGCGGCGGCGGCGGCGGC | | |
| | | GGCGGTGGCGGCTGTTGTTGTTGT | | |
| | | TGTTGTTGTTGTTGTTGTTGGAAGG | | |
| | | ACTTCAGGGATTCGAAGGCTTTCA | | |
| | | TCAGCTTTTCCAATCACTCGCGGTT | | |
| | | CTATCTAGTTACGCGTTAAACCAA | | |
| | | CTAGAA | | |
| htt_h40_GC2_ | 808 | GACCCTGGAAAAGCTGATGAGTTT | epegRNA for | spacer + scaffold + |
| 9CAR4 | | AAGAGCTAGAAATAGCAAGTTTAA | various CAG | flap + linker + 3′motif |
| | | ATAAGGCTAGTCCGTTATCAACTT | replacement, | |
| | | GAAAAAGTGGCACCGAGTCGGTGC | with UA flip | |
| | | GAGGAGGCGGCGGCGGCGGCGGC | | |
| | | GGCGGTGGCGGCTGTTGCTGCTGT | | |
| | | TGCTGTTGCTGTTGCTGTTGGAAG | | |
| | | GACTTCAGGGACTCGAAGGCTTTC | | |
| | | ATCAGCTTTTCCAATCACTCGCGGT | | |
| | | TCTATCTAGTTACGCGTTAAACCA | | |
| | | ACTAGAA | | |
| htt_h40_CT2_ | 809 | GACCCTGGAAAAGCTGATGAGTTT | epegRNA for | spacer + scaffold + |
| 9CAR4 | | AAGAGCTAGAAATAGCAAGTTTAA | various CAG | flap + linker + 3′motif |
| | | ATAAGGCTAGTCCGTTATCAACTT | replacement, | |
| | | GAAAAAGTGGCACCGAGTCGGTGC | with UA flip | |
| | | GAGGAGGCGGCGGCGGCGGCGGC | | |
| | | GGCGGTGGCGGCTGTTGCTGCTGT | | |
| | | TGCTGTTGCTGTTGCTGTTGGAAG | | |
| | | GACTTGAGGGATTCGAAGGCTTTC | | |
| | | ATCAGCTTTTCCAATCACTCGCGGT | | |
| | | TCTATCTAGTTACGCGTTAAACCA | | |
| | | ACTAGAA | | |
| htt_h40_CT2_ | 810 | GACCCTGGAAAAGCTGATGAGTTT | epegRNA for | spacer + scaffold + |
| GC2_9CAR4 | | AAGAGCTAGAAATAGCAAGTTTAA | various CAG | flap + linker + 3′motif |
| | | ATAAGGCTAGTCCGTTATCAACTT | replacement, | |
| | | GAAAAAGTGGCACCGAGTCGGTGC | with UA flip | |
| | | GAGGAGGCGGCGGCGGCGGCGGC | | |
| | | GGCGGTGGCGGCTGTTGCTGCTGT | | |
| | | TGCTGTTGCTGTTGCTGTTGGAAG | | |
| | | GACTTCAGGGATTCGAAGGCTTTC | | |
| | | ATCAGCTTTTCCAATCACTCGCGGT | | |
| | | TCTATCTAGTTACGCGTTAAACCA | | |
| | | ACTAGAA | | |
| htt_h40_GC2_ | 811 | GACCCTGGAAAAGCTGATGAGTTT | epegRNA for | spacer + scaffold + |
| 9CAG | | AAGAGCTAGAAATAGCAAGTTTAA | various CAG | flap + linker + 3′motif |
| | | ATAAGGCTAGTCCGTTATCAACTT | replacement, | |
| | | GAAAAAGTGGCACCGAGTCGGTGC | with UA flip | |
| | | GAGGAGGCGGCGGCGGCGGCGGC | | |
| | | GGCGGTGGCGGCTGTTGCTGCTGC | | |
| | | TGCTGCTGCTGCTGCTGCTGGAAG | | |
| | | GACTTCAGGGACTCGAAGGCTTTC | | |
| | | ATCAGCTTTTCCAATCACTCGCGGT | | |
| | | TCTATCTAGTTACGCGTTAAACCA | | |
| | | ACTAGAA | | |
| htt_h40_CT2_ | 812 | GACCCTGGAAAAGCTGATGAGTTT | epegRNA for | spacer + scaffold + |
| 9CAG | | AAGAGCTAGAAATAGCAAGTTTAA | various CAG | flap + linker + 3′motif |
| | | ATAAGGCTAGTCCGTTATCAACTT | replacement, | |
| | | GAAAAAGTGGCACCGAGTCGGTGC | with UA flip | |
| | | GAGGAGGCGGCGGCGGCGGCGGC | | |
| | | GGCGGTGGCGGCTGTTGCTGCTGC | | |
| | | TGCTGCTGCTGCTGCTGCTGGAAG | | |
| | | GACTTGAGGGATTCGAAGGCTTTC | | |
| | | ATCAGCTTTTCCAATCACTCGCGGT | | |
| | | TCTATCTAGTTACGCGTTAAACCA | | |
| | | ACTAGAA | | |
| htt_h31_ | 813 | GACCCTGGAAAAGCTGATGAGTTT | epegRNA for | spacer + scaffold + |
| 9CAR3 | | AAGAGCTAGAAATAGCAAGTTTAA | various CAG | flap + linker + 3′motif |
| | | ATAAGGCTAGTCCGTTATCAACTT | replacement, | |
| | | GAAAAAGTGGCACCGAGTCGGTGC | with UA flip | |
| | | GCGGCGGCGGCGGCGGCGGTGGC | | |
| | | GGCTGTTGTTGCTGCTGTTGCTGCT | | |
| | | GTTGCTGCTGGAAGGACTTGAGGG | | |
| | | ACTCGAAGGCTTTCATCAGCTTTTC | | |
| | | CAATCACTCGCGGTTCTATCTAGTT | | |
| | | ACGCGTTAAACCAACTAGAA | | |
| htt_h31_ | 814 | GACCCTGGAAAAGCTGATGAGTTT | epegRNA for | spacer + scaffold + |
| 9CAR2 | | AAGAGCTAGAAATAGCAAGTTTAA | various CAG | flap + linker + 3′motif |
| | | ATAAGGCTAGTCCGTTATCAACTT | replacement, | |
| | | GAAAAAGTGGCACCGAGTCGGTGC | with UA flip | |
| | | GCGGCGGCGGCGGCGGCGGTGGC | | |
| | | GGCTGTTGCTGTTGCTGCTGCTGTT | | |
| | | GCTGCTGCTGGAAGGACTTGAGGG | | |
| | | ACTCGAAGGCTTTCATCAGCTTTTC | | |
| | | CAATCACTCGCGGTTCTATCTAGTT | | |
| | | ACGCGTTAAACCAACTAGAA | | |
| htt_h31_ | 815 | GACCCTGGAAAAGCTGATGAGTTT | epegRNA for | spacer + scaffold + |
| 9CAR6 | | AAGAGCTAGAAATAGCAAGTTTAA | various CAG | flap + linker + 3′motif |
| | | ATAAGGCTAGTCCGTTATCAACTT | replacement, | |
| | | GAAAAAGTGGCACCGAGTCGGTGC | with UA flip | |
| | | GCGGCGGCGGCGGCGGCGGTGGC | | |
| | | GGCTGTTGTTGTTGCTGTTGTTGCT | | |
| | | GTTGTTGCTGGAAGGACTTGAGGG | | |
| | | ACTCGAAGGCTTTCATCAGCTTTTC | | |
| | | CAATCACTCGCGGTTCTATCTAGTT | | |
| | | ACGCGTTAAACCAACTAGAA | | |
| HTT exon 1 | 868 | GGGTACCGGCATATGGTTCTTGAC | construct | |
| 72CAG | | AGAGGTGTAAAAAGTACTCAAAA | integrated into | |
| | | ATTTTACTCAAGTGAAAGTACAAG | mESCs for cell | |
| | | TACTTAGGGAAAATTTTACTCAAT | line generation | |
| | | TAAAAGTAAAAGTATCTGGCTAGA | | |
| | | ATCTTACTTGAGTAAAAGTAAAAA | | |
| | | AGTACTCCATTAAAATTGTACTTG | | |
| | | AGTATTAAGGAAGTAAAAGTAAA | | |
| | | AGCAAGAAAGATCGATCTCGAAG | | |
| | | GATCTGGAGGCCACCATGGTGTCG | | |
| | | ATAACTTCGTATAGCATACATTAT | | |
| | | ACGAAGTTATCGTGCTCGACATTG | | |
| | | ATTATTGACTAGTTATTAATAGTA | | |
| | | ATCAATTACGGGGTCATTAGTTCA | | |
| | | TAGCCCATATATGGAGTTCCGCGT | | |
| | | TACATAACTTACGGTAAATGGCCC | | |
| | | GCCTGGCTGACCGCCCAACGACCC | | |
| | | CCGCCCATTGACGTCAATAATGAC | | |
| | | GTATGTTCCCATAGTAACGCCAAT | | |
| | | AGGGACTTTCCATTGACGTCAATG | | |
| | | GGTGGAGTATTTACGGTAAACTGC | | |
| | | CCACTTGGCAGTACATCAAGTGTA | | |
| | | TCATATGCCAAGTACGCCCCCTAT | | |
| | | TGACGTCAATGACGGTAAATGGCC | | |
| | | CGCCTGGCATTATGCCCAGTACAT | | |
| | | GACCTTATGGGACTTTCCTACTTGG | | |
| | | CAGTACATCTACGTATTAGTCATC | | |
| | | GCTATTACCATGGTCGAGGTGAGC | | |
| | | CCCACGTTCTGCTTCACTCTCCCCA | | |
| | | TCTCCCCCCCCTCCCCACCCCCAAT | | |
| | | TTTGTATTTATTTATTTTTTAATTAT | | |
| | | TTTGTGCAGCGATGGGGGCGGGGG | | |
| | | GGGGGGGGGGGGCGCGCCAGGC | | |
| | | GGGGGGGGCGGGGCGAGGGGCG | | |
| | | GGGCGGGGCGAGGCGGAGAGGTG | | |
| | | CGGCGGCAGCCAATCAGAGCGGC | | |
| | | GCGCTCCGAAAGTTTCCTTTTATGG | | |
| | | CGAGGCGGCGGCGGCGGCGGCCCT | | |
| | | ATAAAAAGCGAAGCGCGCGGCGG | | |
| | | GCGGGGAGTCGCTGCGACGCTGCC | | |
| | | TTCGCCCCGTGCCCCGCTCCGCCG | | |
| | | CCGCCTCGCGCCGCCCGCCCCGGC | | |
| | | TCTGACTGACCGCGTTACTCCCAC | | |
| | | AGGTGAGCGGGCGGGACGGCCCTT | | |
| | | CTCCTCCGGGCTGTAATTAGCGCTT | | |
| | | GGTTTAATGACGGCTTGTTTCTTTT | | |
| | | CTGTGGCTGCGTGAAAGCCTTGAG | | |
| | | GGGCTCCGGGAGGGCCCTTTGTGC | | |
| | | GGGGGGAGCGGCTCGGGGGGTGC | | |
| | | GTGCGTGTGTGTGTGCGTGGGGAG | | |
| | | CGCCGCGTGCGGCTCCGCGCTGCC | | |
| | | CGGCGGCTGTGAGCGCTGCGGGCG | | |
| | | CGGCGCGGGGCTTTGTGCGCTCCG | | |
| | | CAGTGTGCGCGAGGGGAGCGCGG | | |
| | | CCGGGGGCGGTGCCCCGCGGTGCG | | |
| | | GGGGGGGCTGCGAGGGGAACAAA | | |
| | | GGCTGCGTGCGGGGTGTGTGCGTG | | |
| | | GGGGGGTGAGCAGGGGGTGTGGG | | |
| | | CGCGTCGGTCGGGCTGCAACCCCC | | |
| | | CCTGCACCCCCCTCCCCGAGTTGCT | | |
| | | GAGCACGGCCCGGCTTCGGGTGCG | | |
| | | GGGCTCCGTACGGGGCGTGGCGCG | | |
| | | GGGCTCGCCGTGCCGGGCGGGGGG | | |
| | | TGGCGGCAGGTGGGGGTGCCGGGC | | |
| | | GGGGCGGGGCCGCCTCGGGCCGG | | |
| | | GGAGGGCTCGGGGGAGGGGCGCG | | |
| | | GCGGCCCCCGGAGCGCCGGCGGCT | | |
| | | GTCGAGGCGCGGCGAGCCGCAGCC | | |
| | | ATTGCCTTTTATGGTAATCGTGCGA | | |
| | | GAGGGCGCAGGGACTTCCTTTGTC | | |
| | | CCAAATCTGTGCGGAGCCGAAATC | | |
| | | TGGGAGGCGCCGCCGCACCCCCTC | | |
| | | TAGCGGGCGCGGGGCGAAGCGGT | | |
| | | GCGGCGCCGGCAGGAAGGAAATG | | |
| | | GGCGGGGAGGGCCTTCGTGCGTCG | | |
| | | CCGCGCCGCCGTCCCCTTCTCCCTC | | |
| | | TCCAGCCTCGGGGCTGTCCGCGGG | | |
| | | GGGACGGCTGCCTTCGGGGGGGAC | | |
| | | GGGGCAGGGCGGGGTTCGGCTTCT | | |
| | | GGCGTGTGACCGGCGGCTCTAGAG | | |
| | | CCTCTGCTAACCATGTTCATGCCTT | | |
| | | CTTCTTTTTCCTACAGCTCCTGGGC | | |
| | | AACGTGCTGGTTATTGTGCTGTCTC | | |
| | | ATCATTTTGGCAAAGAATTCCTCG | | |
| | | AGATGGCGACCCTGGAAAAGCTGA | | |
| | | TGAAGGCCTTCGAGTCCCTCAAGT | | |
| | | CCTTCCAGCAGCAGCAGCAGCAGC | | |
| | | AGCAGCAGCAGCAGCAGCAGCAG | | |
| | | CAGCAGCAGCAGCAGCAGCAGCA | | |
| | | GCAGCAGCAGCAGCAGCAGCAGC | | |
| | | AGCAGCAGCAGCAGCAGCAGCAG | | |
| | | CAGCAGCAGCAGCAGCAGCAGCA | | |
| | | GCAGCAGCAGCAGCAGCAGCAGC | | |
| | | AGCAGCAGCAGCAGCAGCAGCAG | | |
| | | CAGCAGCAGCAGCAGCAGCAGCA | | |
| | | GCAGCAGCAGCAGCAACAGCCGC | | |
| | | CACCGCCGCCGCCGCCGCCGCCGC | | |
| | | CTCCTCAGCTTCCTCAGCCGCCGG | | |
| | | GATCCGCTAGCGCTACTAACTTCA | | |
| | | GCCTGCTGAAGCAGGCTGGAGACG | | |
| | | TGGAGGAGAACCCTGGACCTGGAC | | |
| | | CGGTCGCCACCATGGTGAGCAAGG | | |
| | | GCGAGGAGCTGTTCACCGGGGTGG | | |
| | | TGCCCATCCTGGTCGAGCTGGACG | | |
| | | GCGACGTAAACGGCCACAAGTTCA | | |
| | | GCGTGTCCGGCGAGGGCGAGGGC | | |
| | | GATGCCACCTACGGCAAGCTGACC | | |
| | | CTGAAGTTCATCTGCACCACCGGC | | |
| | | AAGCTGCCCGTGCCCTGGCCCACC | | |
| | | CTCGTGACCACCCTGACCTACGGC | | |
| | | GTGCAGTGCTTCAGCCGCTACCCC | | |
| | | GACCACATGAAGCAGCACGACTTC | | |
| | | TTCAAGTCCGCCATGCCCGAAGGC | | |
| | | TACGTCCAGGAGCGCACCATCTTC | | |
| | | TTCAAGGACGACGGCAACTACAAG | | |
| | | ACCCGCGCCGAGGTGAAGTTCGAG | | |
| | | GGCGACACCCTGGTGAACCGCATC | | |
| | | GAGCTGAAGGGCATCGACTTCAAG | | |
| | | GAGGACGGCAACATCCTGGGGCAC | | |
| | | AAGCTGGAGTACAACTACAACAGC | | |
| | | CACAACGTCTATATCATGGCCGAC | | |
| | | AAGCAGAAGAACGGCATCAAGGT | | |
| | | GAACTTCAAGATCCGCCACAACAT | | |
| | | CGAGGACGGCAGCGTGCAGCTCGC | | |
| | | CGACCACTACCAGCAGAACACCCC | | |
| | | CATCGGCGACGGCCCCGTGCTGCT | | |
| | | GCCCGACAACCACTACCTGAGCAC | | |
| | | CCAGTCCGCCCTGAGCAAAGACCC | | |
| | | CAACGAGAAGCGCGATCACATGGT | | |
| | | CCTGCTGGAGTTCGTGACCGCCGC | | |
| | | CGGGATCACTCTCGGCATGGACGA | | |
| | | GCTGTACAAGTAAAGCGGCCGCCA | | |
| | | CCGCGGTGGAGCTCGAATTAATTC | | |
| | | ATCGATGATGATCCAGACATGATA | | |
| | | AGATACATTGATGAGTTTGGACAA | | |
| | | ACCACAACTAGAATGCAGTGAAAA | | |
| | | AAATGCTTTATTTGTGAAATTTGTG | | |
| | | ATGCTATTGCTTTATTTGTAACCAT | | |
| | | TATAAGCTGCAATAAACAAGTTAA | | |
| | | CAACAACAATTGCATTCATTTTAT | | |
| | | GTTTCAGGTTCAGGGGGAGGTGTG | | |
| | | GGAGGTTTTTTAAAGCAAGTAAAA | | |
| | | CCTCTACAAATGTGGTATGGCTGA | | |
| | | TTATGATCCTCTAGAGTCGGTGGG | | |
| | | CCTCGGGGGCGGGTGCGGGGTCGG | | |
| | | CGGGGCCGCCCCGGGTGGCTTCGG | | |
| | | TCGGAGCCATGGGGTCGTGCGCTC | | |
| | | CTTTCGGTCGGGCGCTGCGGGTCG | | |
| | | TGGGGCGGGCGTCAGGCACCGGGC | | |
| | | TTGCGGGTCATGCACCAGGTGCGC | | |
| | | GGTCCTTCGGGCACCTCGACGTCG | | |
| | | GCGGTGACGGTGAAGCCGAGCCGC | | |
| | | TCGTAGAAGGGGAGGTTGCGGGGC | | |
| | | GCGGAGGTCTCCAGGAAGGCGGG | | |
| | | CACCCCGGCGCGCTCGGCCGCCTC | | |
| | | CACTCCGGGGAGCACGACGGCGCT | | |
| | | GCCCAGACCCTTGCCCTGGTGGTC | | |
| | | GGGCGAGACGCCGACGGTGGCCA | | |
| | | GGAACCACGCGGGCTCCTTGGGCC | | |
| | | GGTGCGGCGCCAGGAGGCCTTCCA | | |
| | | TCTGTTGCTGCGCGGCCAGCCGGG | | |
| | | AACCGCTCAACTCGGCCATGCGCG | | |
| | | GGCCGATCTCGGCGAACACCGCCC | | |
| | | CCGCTTCGACGCTCTCCGGCGTGG | | |
| | | TCCAGACCGCCACCGCGGCGCCGT | | |
| | | CGTCCGCGACCCACACCTTGCCGA | | |
| | | TGTCGAGCCCGACGCGCGTGAGGA | | |
| | | AGAGTTCTTGCAGCTCGGTGACCC | | |
| | | GCTCGATGTGGCGGTCCGGGTCGA | | |
| | | CGGTGTGGCGCGTGGCGGGGTAGT | | |
| | | CGGCGAACGCGGCGGCGAGGGTG | | |
| | | CGTACGGCCCGGGGGACGTCGTCG | | |
| | | CGGGTGGCGAGGCGCACCGTGGGC | | |
| | | TTGTACTCGGTCATGGAAGGTCGT | | |
| | | CTCCTTGTGAGGGGTCAGGGGCGT | | |
| | | GGGTCAGGGGATGGTGGCGGCACC | | |
| | | GGTCGTGGCGGCCGACCTGCAGGC | | |
| | | ATGCAAGCTTTTTGCAAAAGCCTA | | |
| | | GGCCTCCAAAAAAGCCTCCTCACT | | |
| | | ACTTCTGGAATAGCTCAGAGGCCG | | |
| | | AGGCGGCCTCGGCCTCTGCATAAA | | |
| | | TAAAAAAAATTAGTCAGCCATGGG | | |
| | | GCGGAGAATGGGCGGAACTGGGC | | |
| | | GGAGTTAGGGGGGGGATGGGCGG | | |
| | | AGTTAGGGGCGGGACTATGGTTGC | | |
| | | TGACTAATTGAGATGCATGCTTTG | | |
| | | CATACTTCTGCCTGCTGGGGAGCC | | |
| | | TGGGGACTTTCCACACCTGGTTGC | | |
| | | TGACTAATTGAGATGCATGCTTTG | | |
| | | CATACTTCTGCCTGCTGGGGAGCC | | |
| | | TGGGGACTTTCCACACCCTAACTG | | |
| | | ACACACATTCCACAGAATTCAAGT | | |
| | | GATCTCCAAAAAATAAGTACTTTT | | |
| | | TGACTGTAAATAAAATTGTAAGGA | | |
| | | GTAAAAAGTACTTTTTTTTCTAAAA | | |
| | | AAATGTAATTAAGTAAAAGTAAAA | | |
| | | GTATTGATTTTTAATTGTACTCAAG | | |
| | | TAAAGTAAAAATCCCCAAAAATAA | | |
| | | TACTTAAGTACAGTAATCAAGTAA | | |
| | | AATTACTCAAGTACTTTACACCTCT | | |
| | | GG | | |
| HTT exon 1 | 869 | GGGTACCGGCATATGGTTCTTGAC | construct | |
| 21CAG | | AGAGGTGTAAAAAGTACTCAAAA | integrated into | |
| | | ATTTTACTCAAGTGAAAGTACAAG | mESCs for cell | |
| | | TACTTAGGGAAAATTTTACTCAAT | line generation | |
| | | TAAAAGTAAAAGTATCTGGCTAGA | | |
| | | ATCTTACTTGAGTAAAAGTAAAAA | | |
| | | AGTACTCCATTAAAATTGTACTTG | | |
| | | AGTATTAAGGAAGTAAAAGTAAA | | |
| | | AGCAAGAAAGATCGATCTCGAAG | | |
| | | GATCTGGAGGCCACCATGGTGTCG | | |
| | | ATAACTTCGTATAGCATACATTAT | | |
| | | ACGAAGTTATCGTGCTCGACATTG | | |
| | | ATTATTGACTAGTTATTAATAGTA | | |
| | | ATCAATTACGGGGTCATTAGTTCA | | |
| | | TAGCCCATATATGGAGTTCCGCGT | | |
| | | TACATAACTTACGGTAAATGGCCC | | |
| | | GCCTGGCTGACCGCCCAACGACCC | | |
| | | CCGCCCATTGACGTCAATAATGAC | | |
| | | GTATGTTCCCATAGTAACGCCAAT | | |
| | | AGGGACTTTCCATTGACGTCAATG | | |
| | | GGTGGAGTATTTACGGTAAACTGC | | |
| | | CCACTTGGCAGTACATCAAGTGTA | | |
| | | TCATATGCCAAGTACGCCCCCTAT | | |
| | | TGACGTCAATGACGGTAAATGGCC | | |
| | | CGCCTGGCATTATGCCCAGTACAT | | |
| | | GACCTTATGGGACTTTCCTACTTGG | | |
| | | CAGTACATCTACGTATTAGTCATC | | |
| | | GCTATTACCATGGTCGAGGTGAGC | | |
| | | CCCACGTTCTGCTTCACTCTCCCCA | | |
| | | TCTCCCCCCCCTCCCCACCCCCAAT | | |
| | | TTTGTATTTATTTATTTTTTAATTA | | |
| | | TTTTGTGCAGCGATGGGGGCGGGGG | | |
| | | GGGGGGGGGGGGCGCGCCAGGC | | |
| | | GGGGCGGGGCGGGGCGAGGGGCG | | |
| | | GGGCGGGGCGAGGCGGAGAGGTG | | |
| | | CGGCGGCAGCCAATCAGAGCGGC | | |
| | | GCGCTCCGAAAGTTTCCTTTTATGG | | |
| | | CGAGGCGGCGGCGGCGGCGGCCCT | | |
| | | ATAAAAAGCGAAGCGCGCGGCGG | | |
| | | GCGGGGAGTCGCTGCGACGCTGCC | | |
| | | TTCGCCCCGTGCCCCGCTCCGCCG | | |
| | | CCGCCTCGCGCCGCCCGCCCCGGC | | |
| | | TCTGACTGACCGCGTTACTCCCAC | | |
| | | AGGTGAGCGGGCGGGACGGCCCTT | | |
| | | CTCCTCCGGGCTGTAATTAGCGCTT | | |
| | | GGTTTAATGACGGCTTGTTTCTTTT | | |
| | | CTGTGGCTGCGTGAAAGCCTTGAG | | |
| | | GGGCTCCGGGAGGGCCCTTTGTGC | | |
| | | GGGGGGAGCGGCTCGGGGGGTGC | | |
| | | GTGCGTGTGTGTGTGCGTGGGGAG | | |
| | | CGCCGCGTGCGGCTCCGCGCTGCC | | |
| | | CGGCGGCTGTGAGCGCTGCGGGCG | | |
| | | CGGCGCGGGGCTTTGTGCGCTCCG | | |
| | | CAGTGTGCGCGAGGGGAGCGCGG | | |
| | | CCGGGGGCGGTGCCCCGCGGTGCG | | |
| | | GGGGGGGCTGCGAGGGGAACAAA | | |
| | | GGCTGCGTGCGGGGTGTGTGCGTG | | |
| | | GGGGGGTGAGCAGGGGGTGTGGG | | |
| | | CGCGTCGGTCGGGCTGCAACCCCC | | |
| | | CCTGCACCCCCCTCCCCGAGTTGCT | | |
| | | GAGCACGGCCCGGCTTCGGGTGCG | | |
| | | GGGCTCCGTACGGGGCGTGGCGCG | | |
| | | GGGCTCGCCGTGCCGGGGGGGGG | | |
| | | TGGCGGCAGGTGGGGGTGCCGGGC | | |
| | | GGGGCGGGGCCGCCTCGGGCCGG | | |
| | | GGAGGGCTCGGGGGAGGGGCGCG | | |
| | | GCGGCCCCCGGAGCGCCGGCGGCT | | |
| | | GTCGAGGCGCGGCGAGCCGCAGCC | | |
| | | ATTGCCTTTTATGGTAATCGTGCGA | | |
| | | GAGGGCGCAGGGACTTCCTTTGTC | | |
| | | CCAAATCTGTGCGGAGCCGAAATC | | |
| | | TGGGAGGCGCCGCCGCACCCCCTC | | |
| | | TAGCGGGCGCGGGGCGAAGCGGT | | |
| | | GCGGCGCCGGCAGGAAGGAAATG | | |
| | | GGCGGGGAGGGCCTTCGTGCGTCG | | |
| | | CCGCGCCGCCGTCCCCTTCTCCCTC | | |
| | | TCCAGCCTCGGGGCTGTCCGCGGG | | |
| | | GGGACGGCTGCCTTCGGGGGGGAC | | |
| | | GGGGCAGGGCGGGGTTCGGCTTCT | | |
| | | GGCGTGTGACCGGCGGCTCTAGAG | | |
| | | CCTCTGCTAACCATGTTCATGCCTT | | |
| | | CTTCTTTTTCCTACAGCTCCTGGGC | | |
| | | AACGTGCTGGTTATTGTGCTGTCTC | | |
| | | ATCATTTTGGCAAAGAATTCCTCG | | |
| | | AGATGGCGACCCTGGAAAAGCTGA | | |
| | | TGAAGGCCTTCGAGTCCCTCAAGT | | |
| | | CCTTCCAGCAGCAGCAGCAGCAGC | | |
| | | AGCAGCAGCAGCAGCAGCAGCAG | | |
| | | CAGCAGCAGCAGCAGCAGCAGCA | | |
| | | ACAGCCGCCACCGCCGCCGCCGCC | | |
| | | GCCGCCGCCTCCTCAGCTTCCTCA | | |
| | | GCCGCCGGGATCCGCTAGCGCTAC | | |
| | | TAACTTCAGCCTGCTGAAGCAGGC | | |
| | | TGGAGACGTGGAGGAGAACCCTG | | |
| | | GACCTGGACCGGTCGCCACCATGG | | |
| | | TGAGCAAGGGCGAGGAGCTGTTCA | | |
| | | CCGGGGTGGTGCCCATCCTGGTCG | | |
| | | AGCTGGACGGCGACGTAAACGGCC | | |
| | | ACAAGTTCAGCGTGTCCGGCGAGG | | |
| | | GCGAGGGCGATGCCACCTACGGCA | | |
| | | AGCTGACCCTGAAGTTCATCTGCA | | |
| | | CCACCGGCAAGCTGCCCGTGCCCT | | |
| | | GGCCCACCCTCGTGACCACCCTGA | | |
| | | CCTACGGCGTGCAGTGCTTCAGCC | | |
| | | GCTACCCCGACCACATGAAGCAGC | | |
| | | ACGACTTCTTCAAGTCCGCCATGC | | |
| | | CCGAAGGCTACGTCCAGGAGCGCA | | |
| | | CCATCTTCTTCAAGGACGACGGCA | | |
| | | ACTACAAGACCCGCGCCGAGGTGA | | |
| | | AGTTCGAGGGCGACACCCTGGTGA | | |
| | | ACCGCATCGAGCTGAAGGGCATCG | | |
| | | ACTTCAAGGAGGACGGCAACATCC | | |
| | | TGGGGCACAAGCTGGAGTACAACT | | |
| | | ACAACAGCCACAACGTCTATATCA | | |
| | | TGGCCGACAAGCAGAAGAACGGC | | |
| | | ATCAAGGTGAACTTCAAGATCCGC | | |
| | | CACAACATCGAGGACGGCAGCGTG | | |
| | | CAGCTCGCCGACCACTACCAGCAG | | |
| | | AACACCCCCATCGGCGACGGCCCC | | |
| | | GTGCTGCTGCCCGACAACCACTAC | | |
| | | CTGAGCACCCAGTCCGCCCTGAGC | | |
| | | AAAGACCCCAACGAGAAGCGCGA | | |
| | | TCACATGGTCCTGCTGGAGTTCGT | | |
| | | GACCGCCGCCGGGATCACTCTCGG | | |
| | | CATGGACGAGCTGTACAAGTAAAG | | |
| | | CGGCCGCCACCGCGGTGGAGCTCG | | |
| | | AATTAATTCATCGATGATGATCCA | | |
| | | GACATGATAAGATACATTGATGAG | | |
| | | TTTGGACAAACCACAACTAGAATG | | |
| | | CAGTGAAAAAAATGCTTTATTTGT | | |
| | | GAAATTTGTGATGCTATTGCTTTAT | | |
| | | TTGTAACCATTATAAGCTGCAATA | | |
| | | AACAAGTTAACAACAACAATTGCA | | |
| | | TTCATTTTATGTTTCAGGTTCAGGG | | |
| | | GGAGGTGTGGGAGGTTTTTTAAAG | | |
| | | CAAGTAAAACCTCTACAAATGTGG | | |
| | | TATGGCTGATTATGATCCTCTAGA | | |
| | | GTCGGTGGGCCTCGGGGGCGGGTG | | |
| | | CGGGGTCGGCGGGGCCGCCCCGGG | | |
| | | TGGCTTCGGTCGGAGCCATGGGGT | | |
| | | CGTGCGCTCCTTTCGGTCGGGCGC | | |
| | | TGCGGGTCGTGGGGGGGGCGTCAG | | |
| | | GCACCGGGCTTGCGGGTCATGCAC | | |
| | | CAGGTGCGCGGTCCTTCGGGCACC | | |
| | | TCGACGTCGGCGGTGACGGTGAAG | | |
| | | CCGAGCCGCTCGTAGAAGGGGAG | | |
| | | GTTGCGGGGCGCGGAGGTCTCCAG | | |
| | | GAAGGCGGGCACCCCGGCGCGCTC | | |
| | | GGCCGCCTCCACTCCGGGGAGCAC | | |
| | | GACGGCGCTGCCCAGACCCTTGCC | | |
| | | CTGGTGGTCGGGCGAGACGCCGAC | | |
| | | GGTGGCCAGGAACCACGCGGGCTC | | |
| | | CTTGGGCCGGTGCGGCGCCAGGAG | | |
| | | GCCTTCCATCTGTTGCTGCGCGGCC | | |
| | | AGCCGGGAACCGCTCAACTCGGCC | | |
| | | ATGCGCGGGCCGATCTCGGCGAAC | | |
| | | ACCGCCCCCGCTTCGACGCTCTCC | | |
| | | GGCGTGGTCCAGACCGCCACCGCG | | |
| | | GCGCCGTCGTCCGCGACCCACACC | | |
| | | TTGCCGATGTCGAGCCCGACGCGC | | |
| | | GTGAGGAAGAGTTCTTGCAGCTCG | | |
| | | GTGACCCGCTCGATGTGGCGGTCC | | |
| | | GGGTCGACGGTGTGGCGCGTGGCG | | |
| | | GGGTAGTCGGCGAACGCGGCGGC | | |
| | | GAGGGTGCGTACGGCCCGGGGGA | | |
| | | CGTCGTCGCGGGTGGCGAGGCGCA | | |
| | | CCGTGGGCTTGTACTCGGTCATGG | | |
| | | AAGGTCGTCTCCTTGTGAGGGGTC | | |
| | | AGGGGCGTGGGTCAGGGGATGGT | | |
| | | GGCGGCACCGGTCGTGGCGGCCGA | | |
| | | CCTGCAGGCATGCAAGCTTTTTGC | | |
| | | AAAAGCCTAGGCCTCCAAAAAAGC | | |
| | | CTCCTCACTACTTCTGGAATAGCTC | | |
| | | AGAGGCCGAGGCGGCCTCGGCCTC | | |
| | | TGCATAAATAAAAAAAATTAGTCA | | |
| | | GCCATGGGGCGGAGAATGGGCGG | | |
| | | AACTGGGCGGAGTTAGGGGGGGG | | |
| | | ATGGGCGGAGTTAGGGGGGGGACT | | |
| | | ATGGTTGCTGACTAATTGAGATGC | | |
| | | ATGCTTTGCATACTTCTGCCTGCTG | | |
| | | GGGAGCCTGGGGACTTTCCACACC | | |
| | | TGGTTGCTGACTAATTGAGATGCA | | |
| | | TGCTTTGCATACTTCTGCCTGCTGG | | |
| | | GGAGCCTGGGGACTTTCCACACCC | | |
| | | TAACTGACACACATTCCACAGAAT | | |
| | | TCAAGTGATCTCCAAAAAATAAGT | | |
| | | ACTTTTTGACTGTAAATAAAATTGT | | |
| | | AAGGAGTAAAAAGTACTTTTTTTT | | |
| | | CTAAAAAAATGTAATTAAGTAAAA | | |
| | | GTAAAAGTATTGATTTTTAATTGTA | | |
| | | CTCAAGTAAAGTAAAAATCCCCAA | | |
| | | AAATAATACTTAAGTACAGTAATC | | |
| | | AAGTAAAATTACTCAAGTACTTTA | | |
| | | CACCTCTGG | | |
| HTT exon 1 | 870 | GGGTACCGGCATATGGTTCTTGAC | construct | |
| 72CAG, CCG | | AGAGGTGTAAAAAGTACTCAAAA | integrated into | |
| interruptions | | ATTTTACTCAAGTGAAAGTACAAG | mESCs for cell | |
| | | TACTTAGGGAAAATTTTACTCAAT | line generation | |
| | | TAAAAGTAAAAGTATCTGGCTAGA | | |
| | | ATCTTACTTGAGTAAAAGTAAAAA | | |
| | | AGTACTCCATTAAAATTGTACTTG | | |
| | | AGTATTAAGGAAGTAAAAGTAAA | | |
| | | AGCAAGAAAGATCGATCTCGAAG | | |
| | | GATCTGGAGGCCACCATGGTGTCG | | |
| | | ATAACTTCGTATAGCATACATTAT | | |
| | | ACGAAGTTATCGTGCTCGACATTG | | |
| | | ATTATTGACTAGTTATTAATAGTA | | |
| | | ATCAATTACGGGGTCATTAGTTCA | | |
| | | TAGCCCATATATGGAGTTCCGCGT | | |
| | | TACATAACTTACGGTAAATGGCCC | | |
| | | GCCTGGCTGACCGCCCAACGACCC | | |
| | | CCGCCCATTGACGTCAATAATGAC | | |
| | | GTATGTTCCCATAGTAACGCCAAT | | |
| | | AGGGACTTTCCATTGACGTCAATG | | |
| | | GGTGGAGTATTTACGGTAAACTGC | | |
| | | CCACTTGGCAGTACATCAAGTGTA | | |
| | | TCATATGCCAAGTACGCCCCCTAT | | |
| | | TGACGTCAATGACGGTAAATGGCC | | |
| | | CGCCTGGCATTATGCCCAGTACAT | | |
| | | GACCTTATGGGACTTTCCTACTTGG | | |
| | | CAGTACATCTACGTATTAGTCATC | | |
| | | GCTATTACCATGGTCGAGGTGAGC | | |
| | | CCCACGTTCTGCTTCACTCTCCCCA | | |
| | | TCTCCCCCCCCTCCCCACCCCCAAT | | |
| | | TTTGTATTTATTTATTTTTTAATTA | | |
| | | TTTTGTGCAGCGATGGGGGCGGGGG | | |
| | | GGGGGGGGGGGGCGCGCCAGGC | | |
| | | GGGGCGGGGCGGGGCGAGGGGCG | | |
| | | GGGCGGGGCGAGGCGGAGAGGTG | | |
| | | CGGCGGCAGCCAATCAGAGCGGC | | |
| | | GCGCTCCGAAAGTTTCCTTTTATGG | | |
| | | CGAGGCGGCGGCGGCGGCGGCCCT | | |
| | | ATAAAAAGCGAAGCGCGCGGCGG | | |
| | | GCGGGGAGTCGCTGCGACGCTGCC | | |
| | | TTCGCCCCGTGCCCCGCTCCGCCG | | |
| | | CCGCCTCGCGCCGCCCGCCCCGGC | | |
| | | TCTGACTGACCGCGTTACTCCCAC | | |
| | | AGGTGAGCGGGCGGGACGGCCCTT | | |
| | | CTCCTCCGGGCTGTAATTAGCGCTT | | |
| | | GGTTTAATGACGGCTTGTTTCTTTT | | |
| | | CTGTGGCTGCGTGAAAGCCTTGAG | | |
| | | GGGCTCCGGGAGGGCCCTTTGTGC | | |
| | | GGGGGGAGCGGCTCGGGGGGTGC | | |
| | | GTGCGTGTGTGTGTGCGTGGGGAG | | |
| | | CGCCGCGTGCGGCTCCGCGCTGCC | | |
| | | CGGCGGCTGTGAGCGCTGCGGGCG | | |
| | | CGGCGCGGGGCTTTGTGCGCTCCG | | |
| | | CAGTGTGCGCGAGGGGAGCGCGG | | |
| | | CCGGGGGCGGTGCCCCGCGGTGCG | | |
| | | GGGGGGGCTGCGAGGGGAACAAA | | |
| | | GGCTGCGTGCGGGGTGTGTGCGTG | | |
| | | GGGGGGTGAGCAGGGGGTGTGGG | | |
| | | CGCGTCGGTCGGGCTGCAACCCCC | | |
| | | CCTGCACCCCCCTCCCCGAGTTGCT | | |
| | | GAGCACGGCCCGGCTTCGGGTGCG | | |
| | | GGGCTCCGTACGGGGCGTGGCGCG | | |
| | | GGGCTCGCCGTGCCGGGCGGGGGG | | |
| | | TGGCGGCAGGTGGGGGTGCCGGGC | | |
| | | GGGGCGGGGCCGCCTCGGGCCGG | | |
| | | GGAGGGCTCGGGGGAGGGGCGCG | | |
| | | GCGGCCCCCGGAGCGCCGGCGGCT | | |
| | | GTCGAGGCGCGGCGAGCCGCAGCC | | |
| | | ATTGCCTTTTATGGTAATCGTGCGA | | |
| | | GAGGGCGCAGGGACTTCCTTTGTC | | |
| | | CCAAATCTGTGCGGAGCCGAAATC | | |
| | | TGGGAGGCGCCGCCGCACCCCCTC | | |
| | | TAGCGGGCGCGGGGCGAAGCGGT | | |
| | | GCGGCGCCGGCAGGAAGGAAATG | | |
| | | GGCGGGGAGGGCCTTCGTGCGTCG | | |
| | | CCGCGCCGCCGTCCCCTTCTCCCTC | | |
| | | TCCAGCCTCGGGGCTGTCCGCGGG | | |
| | | GGGACGGCTGCCTTCGGGGGGGAC | | |
| | | GGGGCAGGGCGGGGTTCGGCTTCT | | |
| | | GGCGTGTGACCGGCGGCTCTAGAG | | |
| | | CCTCTGCTAACCATGTTCATGCCTT | | |
| | | CTTCTTTTTCCTACAGCTCCTGGGC | | |
| | | AACGTGCTGGTTATTGTGCTGTCTC | | |
| | | ATCATTTTGGCAAAGAATTCCTCG | | |
| | | AGATGGCGACCCTGGAAAAGCTGA | | |
| | | TGAAGGCCTTCGAGTCCCTCAAGT | | |
| | | CCTTCCAGCAGCAGCAGCAGCCGC | | |
| | | AGCAGCAGCAGCAGCAGCAGCAG | | |
| | | CAGCAGCAGCAGCAGCAGCAGCA | | |
| | | GCAGCAGCAGCAGCAGCAGCAGC | | |
| | | AGCAGCAGCCGCAGCAGCAGCAG | | |
| | | CAGCAGCAGCAGCAGCAGCAGCC | | |
| | | GCAGCAGCAGCAGCAGCAGCAGC | | |
| | | AGCAGCAGCAGCAGCAGCAGCAG | | |
| | | CAGCAGCAGCAGCAGCAGCAGCA | | |
| | | GCAGCAGCAGCAACAGCCGCCACC | | |
| | | GCCGCCGCCGCCGCCGCCGCCTCC | | |
| | | TCAGCTTCCTCAGCCGCCGGGATC | | |
| | | CGCTAGCGCTACTAACTTCAGCCT | | |
| | | GCTGAAGCAGGCTGGAGACGTGG | | |
| | | AGGAGAACCCTGGACCTGGACCGG | | |
| | | TCGCCACCATGGTGAGCAAGGGCG | | |
| | | AGGAGCTGTTCACCGGGGTGGTGC | | |
| | | CCATCCTGGTCGAGCTGGACGGCG | | |
| | | ACGTAAACGGCCACAAGTTCAGCG | | |
| | | TGTCCGGCGAGGGCGAGGGCGATG | | |
| | | CCACCTACGGCAAGCTGACCCTGA | | |
| | | AGTTCATCTGCACCACCGGCAAGC | | |
| | | TGCCCGTGCCCTGGCCCACCCTCG | | |
| | | TGACCACCCTGACCTACGGCGTGC | | |
| | | AGTGCTTCAGCCGCTACCCCGACC | | |
| | | ACATGAAGCAGCACGACTTCTTCA | | |
| | | AGTCCGCCATGCCCGAAGGCTACG | | |
| | | TCCAGGAGCGCACCATCTTCTTCA | | |
| | | AGGACGACGGCAACTACAAGACC | | |
| | | CGCGCCGAGGTGAAGTTCGAGGGC | | |
| | | GACACCCTGGTGAACCGCATCGAG | | |
| | | CTGAAGGGCATCGACTTCAAGGAG | | |
| | | GACGGCAACATCCTGGGGCACAAG | | |
| | | CTGGAGTACAACTACAACAGCCAC | | |
| | | AACGTCTATATCATGGCCGACAAG | | |
| | | CAGAAGAACGGCATCAAGGTGAA | | |
| | | CTTCAAGATCCGCCACAACATCGA | | |
| | | GGACGGCAGCGTGCAGCTCGCCGA | | |
| | | CCACTACCAGCAGAACACCCCCAT | | |
| | | CGGCGACGGCCCCGTGCTGCTGCC | | |
| | | CGACAACCACTACCTGAGCACCCA | | |
| | | GTCCGCCCTGAGCAAAGACCCCAA | | |
| | | CGAGAAGCGCGATCACATGGTCCT | | |
| | | GCTGGAGTTCGTGACCGCCGCCGG | | |
| | | GATCACTCTCGGCATGGACGAGCT | | |
| | | GTACAAGTAAAGCGGCCGCCACCG | | |
| | | CGGTGGAGCTCGAATTAATTCATC | | |
| | | GATGATGATCCAGACATGATAAGA | | |
| | | TACATTGATGAGTTTGGACAAACC | | |
| | | ACAACTAGAATGCAGTGAAAAAA | | |
| | | ATGCTTTATTTGTGAAATTTGTGAT | | |
| | | GCTATTGCTTTATTTGTAACCATTA | | |
| | | TAAGCTGCAATAAACAAGTTAACA | | |
| | | ACAACAATTGCATTCATTTTATGTT | | |
| | | TCAGGTTCAGGGGGAGGTGTGGGA | | |
| | | GGTTTTTTAAAGCAAGTAAAACCT | | |
| | | CTACAAATGTGGTATGGCTGATTA | | |
| | | TGATCCTCTAGAGTCGGTGGGCCT | | |
| | | CGGGGGCGGGTGCGGGGTCGGCG | | |
| | | GGGCCGCCCCGGGTGGCTTCGGTC | | |
| | | GGAGCCATGGGGTCGTGCGCTCCT | | |
| | | TTCGGTCGGGCGCTGCGGGTCGTG | | |
| | | GGGCGGGCGTCAGGCACCGGGCTT | | |
| | | GCGGGTCATGCACCAGGTGCGCGG | | |
| | | TCCTTCGGGCACCTCGACGTCGGC | | |
| | | GGTGACGGTGAAGCCGAGCCGCTC | | |
| | | GTAGAAGGGGAGGTTGCGGGGCG | | |
| | | CGGAGGTCTCCAGGAAGGCGGGC | | |
| | | ACCCCGGCGCGCTCGGCCGCCTCC | | |
| | | ACTCCGGGGAGCACGACGGCGCTG | | |
| | | CCCAGACCCTTGCCCTGGTGGTCG | | |
| | | GGCGAGACGCCGACGGTGGCCAG | | |
| | | GAACCACGCGGGCTCCTTGGGCCG | | |
| | | GTGCGGCGCCAGGAGGCCTTCCAT | | |
| | | CTGTTGCTGCGCGGCCAGCCGGGA | | |
| | | ACCGCTCAACTCGGCCATGCGCGG | | |
| | | GCCGATCTCGGCGAACACCGCCCC | | |
| | | CGCTTCGACGCTCTCCGGCGTGGT | | |
| | | CCAGACCGCCACCGCGGCGCCGTC | | |
| | | GTCCGCGACCCACACCTTGCCGAT | | |
| | | GTCGAGCCCGACGCGCGTGAGGAA | | |
| | | GAGTTCTTGCAGCTCGGTGACCCG | | |
| | | CTCGATGTGGCGGTCCGGGTCGAC | | |
| | | GGTGTGGCGCGTGGCGGGGTAGTC | | |
| | | GGCGAACGCGGCGGCGAGGGTGC | | |
| | | GTACGGCCCGGGGGACGTCGTCGC | | |
| | | GGGTGGCGAGGCGCACCGTGGGCT | | |
| | | TGTACTCGGTCATGGAAGGTCGTC | | |
| | | TCCTTGTGAGGGGTCAGGGGCGTG | | |
| | | GGTCAGGGGATGGTGGCGGCACCG | | |
| | | GTCGTGGCGGCCGACCTGCAGGCA | | |
| | | TGCAAGCTTTTTGCAAAAGCCTAG | | |
| | | GCCTCCAAAAAAGCCTCCTCACTA | | |
| | | CTTCTGGAATAGCTCAGAGGCCGA | | |
| | | GGCGGCCTCGGCCTCTGCATAAAT | | |
| | | AAAAAAAATTAGTCAGCCATGGGG | | |
| | | CGGAGAATGGGCGGAACTGGGCG | | |
| | | GAGTTAGGGGGGGGATGGGCGGA | | |
| | | GTTAGGGGCGGGACTATGGTTGCT | | |
| | | GACTAATTGAGATGCATGCTTTGC | | |
| | | ATACTTCTGCCTGCTGGGGAGCCT | | |
| | | GGGGACTTTCCACACCTGGTTGCT | | |
| | | GACTAATTGAGATGCATGCTTTGC | | |
| | | ATACTTCTGCCTGCTGGGGAGCCT | | |
| | | GGGGACTTTCCACACCCTAACTGA | | |
| | | CACACATTCCACAGAATTCAAGTG | | |
| | | ATCTCCAAAAAATAAGTACTTTTT | | |
| | | GACTGTAAATAAAATTGTAAGGAG | | |
| | | TAAAAAGTACTTTTTTTTCTAAAAA | | |
| | | AATGTAATTAAGTAAAAGTAAAAG | | |
| | | TATTGATTTTTAATTGTACTCAAGT | | |
| | | AAAGTAAAAATCCCCAAAAATAAT | | |
| | | ACTTAAGTACAGTAATCAAGTAAA | | |
| | | ATTACTCAAGTACTTTACACCTCTG | | |
| HTT exon 1 | 871 | GGGTACCGGCATATGGTTCTTGAC | construct | |
| 72CAG, CAA | | AGAGGTGTAAAAAGTACTCAAAA | integrated into | |
| interruptions | | ATTTTACTCAAGTGAAAGTACAAG | mESCs for cell | |
| | | TACTTAGGGAAAATTTTACTCAAT | line generation | |
| | | TAAAAGTAAAAGTATCTGGCTAGA | | |
| | | ATCTTACTTGAGTAAAAGTAAAAA | | |
| | | AGTACTCCATTAAAATTGTACTTG | | |
| | | AGTATTAAGGAAGTAAAAGTAAA | | |
| | | AGCAAGAAAGATCGATCTCGAAG | | |
| | | GATCTGGAGGCCACCATGGTGTCG | | |
| | | ATAACTTCGTATAGCATACATTAT | | |
| | | ACGAAGTTATCGTGCTCGACATTG | | |
| | | ATTATTGACTAGTTATTAATAGTA | | |
| | | ATCAATTACGGGGTCATTAGTTCA | | |
| | | TAGCCCATATATGGAGTTCCGCGT | | |
| | | TACATAACTTACGGTAAATGGCCC | | |
| | | GCCTGGCTGACCGCCCAACGACCC | | |
| | | CCGCCCATTGACGTCAATAATGAC | | |
| | | GTATGTTCCCATAGTAACGCCAAT | | |
| | | AGGGACTTTCCATTGACGTCAATG | | |
| | | GGTGGAGTATTTACGGTAAACTGC | | |
| | | CCACTTGGCAGTACATCAAGTGTA | | |
| | | TCATATGCCAAGTACGCCCCCTAT | | |
| | | TGACGTCAATGACGGTAAATGGCC | | |
| | | CGCCTGGCATTATGCCCAGTACAT | | |
| | | GACCTTATGGGACTTTCCTACTTGG | | |
| | | CAGTACATCTACGTATTAGTCATC | | |
| | | GCTATTACCATGGTCGAGGTGAGC | | |
| | | CCCACGTTCTGCTTCACTCTCCCCA | | |
| | | TCTCCCCCCCCTCCCCACCCCCAAT | | |
| | | TTTGTATTTATTTATTTTTTAATTA | | |
| | | TTTTGTGCAGCGATGGGGGCGGGGG | | |
| | | GGGGGGGGGGGGCGCGCCAGGC | | |
| | | GGGGGGGGCGGGGCGAGGGGCG | | |
| | | GGGCGGGGCGAGGCGGAGAGGTG | | |
| | | CGGCGGCAGCCAATCAGAGCGGC | | |
| | | GCGCTCCGAAAGTTTCCTTTTATGG | | |
| | | CGAGGCGGCGGCGGCGGCGGCCCT | | |
| | | ATAAAAAGCGAAGCGCGCGGCGG | | |
| | | GCGGGGAGTCGCTGCGACGCTGCC | | |
| | | TTCGCCCCGTGCCCCGCTCCGCCG | | |
| | | CCGCCTCGCGCCGCCCGCCCCGGC | | |
| | | TCTGACTGACCGCGTTACTCCCAC | | |
| | | AGGTGAGCGGGCGGGACGGCCCTT | | |
| | | CTCCTCCGGGCTGTAATTAGCGCTT | | |
| | | GGTTTAATGACGGCTTGTTTCTTTT | | |
| | | CTGTGGCTGCGTGAAAGCCTTGAG | | |
| | | GGGCTCCGGGAGGGCCCTTTGTGC | | |
| | | GGGGGGAGCGGCTCGGGGGGTGC | | |
| | | GTGCGTGTGTGTGTGCGTGGGGAG | | |
| | | CGCCGCGTGCGGCTCCGCGCTGCC | | |
| | | CGGCGGCTGTGAGCGCTGCGGGCG | | |
| | | CGGCGCGGGGCTTTGTGCGCTCCG | | |
| | | CAGTGTGCGCGAGGGGAGCGCGG | | |
| | | CCGGGGGCGGTGCCCCGCGGTGCG | | |
| | | GGGGGGGCTGCGAGGGGAACAAA | | |
| | | GGCTGCGTGCGGGGTGTGTGCGTG | | |
| | | GGGGGGTGAGCAGGGGGTGTGGG | | |
| | | CGCGTCGGTCGGGCTGCAACCCCC | | |
| | | CCTGCACCCCCCTCCCCGAGTTGCT | | |
| | | GAGCACGGCCCGGCTTCGGGTGCG | | |
| | | GGGCTCCGTACGGGGCGTGGCGCG | | |
| | | GGGCTCGCCGTGCCGGGCGGGGGG | | |
| | | TGGCGGCAGGTGGGGGTGCCGGGC | | |
| | | GGGGCGGGGCCGCCTCGGGCCGG | | |
| | | GGAGGGCTCGGGGGAGGGGCGCG | | |
| | | GCGGCCCCCGGAGCGCCGGCGGCT | | |
| | | GTCGAGGCGCGGCGAGCCGCAGCC | | |
| | | ATTGCCTTTTATGGTAATCGTGCGA | | |
| | | GAGGGCGCAGGGACTTCCTTTGTC | | |
| | | CCAAATCTGTGCGGAGCCGAAATC | | |
| | | TGGGAGGCGCCGCCGCACCCCCTC | | |
| | | TAGCGGGCGCGGGGCGAAGCGGT | | |
| | | GCGGCGCCGGCAGGAAGGAAATG | | |
| | | GGCGGGGAGGGCCTTCGTGCGTCG | | |
| | | CCGCGCCGCCGTCCCCTTCTCCCTC | | |
| | | TCCAGCCTCGGGGCTGTCCGCGGG | | |
| | | GGGACGGCTGCCTTCGGGGGGGAC | | |
| | | GGGGCAGGGCGGGGTTCGGCTTCT | | |
| | | GGCGTGTGACCGGCGGCTCTAGAG | | |
| | | CCTCTGCTAACCATGTTCATGCCTT | | |
| | | CTTCTTTTTCCTACAGCTCCTGGGC | | |
| | | AACGTGCTGGTTATTGTGCTGTCTC | | |
| | | ATCATTTTGGCAAAGAATTCCTCG | | |
| | | AGATGGCGACCCTGGAAAAGCTGA | | |
| | | TGAAGGCCTTCGAGTCCCTCAAGT | | |
| | | CCTTCCAGCAGCAGCAGCAGCAGC | | |
| | | AGCAACAGCAGCAGCAGCAGCAG | | |
| | | CAGCAACAGCAGCAGCAGCAGCA | | |
| | | GCAGCAGCAGCAGCAGCAGCAGC | | |
| | | AGCAGCAGCAGCAGCAGCAGCAG | | |
| | | CAGCAGCAGCAACAGCAGCAGCA | | |
| | | GCAGCAGCAGCAGCAGCAGCAGC | | |
| | | AGCAGCAGCAGCAGCAGCAGCAG | | |
| | | CAGCAGCAGCAGCAACAGCAGCA | | |
| | | GCAGCAGCAGCAGCAACAGCCGC | | |
| | | CACCGCCGCCGCCGCCGCCGCCGC | | |
| | | CTCCTCAGCTTCCTCAGCCGCCGG | | |
| | | GATCCGCTAGCGCTACTAACTTCA | | |
| | | GCCTGCTGAAGCAGGCTGGAGACG | | |
| | | TGGAGGAGAACCCTGGACCTGGAC | | |
| | | CGGTCGCCACCATGGTGAGCAAGG | | |
| | | GCGAGGAGCTGTTCACCGGGGTGG | | |
| | | TGCCCATCCTGGTCGAGCTGGACG | | |
| | | GCGACGTAAACGGCCACAAGTTCA | | |
| | | GCGTGTCCGGCGAGGGCGAGGGC | | |
| | | GATGCCACCTACGGCAAGCTGACC | | |
| | | CTGAAGTTCATCTGCACCACCGGC | | |
| | | AAGCTGCCCGTGCCCTGGCCCACC | | |
| | | CTCGTGACCACCCTGACCTACGGC | | |
| | | GTGCAGTGCTTCAGCCGCTACCCC | | |
| | | GACCACATGAAGCAGCACGACTTC | | |
| | | TTCAAGTCCGCCATGCCCGAAGGC | | |
| | | TACGTCCAGGAGCGCACCATCTTC | | |
| | | TTCAAGGACGACGGCAACTACAAG | | |
| | | ACCCGCGCCGAGGTGAAGTTCGAG | | |
| | | GGCGACACCCTGGTGAACCGCATC | | |
| | | GAGCTGAAGGGCATCGACTTCAAG | | |
| | | GAGGACGGCAACATCCTGGGGCAC | | |
| | | AAGCTGGAGTACAACTACAACAGC | | |
| | | CACAACGTCTATATCATGGCCGAC | | |
| | | AAGCAGAAGAACGGCATCAAGGT | | |
| | | GAACTTCAAGATCCGCCACAACAT | | |
| | | CGAGGACGGCAGCGTGCAGCTCGC | | |
| | | CGACCACTACCAGCAGAACACCCC | | |
| | | CATCGGCGACGGCCCCGTGCTGCT | | |
| | | GCCCGACAACCACTACCTGAGCAC | | |
| | | CCAGTCCGCCCTGAGCAAAGACCC | | |
| | | CAACGAGAAGCGCGATCACATGGT | | |
| | | CCTGCTGGAGTTCGTGACCGCCGC | | |
| | | CGGGATCACTCTCGGCATGGACGA | | |
| | | GCTGTACAAGTAAAGCGGCCGCCA | | |
| | | CCGCGGTGGAGCTCGAATTAATTC | | |
| | | ATCGATGATGATCCAGACATGATA | | |
| | | AGATACATTGATGAGTTTGGACAA | | |
| | | ACCACAACTAGAATGCAGTGAAAA | | |
| | | AAATGCTTTATTTGTGAAATTTGTG | | |
| | | ATGCTATTGCTTTATTTGTAACCAT | | |
| | | TATAAGCTGCAATAAACAAGTTAA | | |
| | | CAACAACAATTGCATTCATTTTAT | | |
| | | GTTTCAGGTTCAGGGGGAGGTGTG | | |
| | | GGAGGTTTTTTAAAGCAAGTAAAA | | |
| | | CCTCTACAAATGTGGTATGGCTGA | | |
| | | TTATGATCCTCTAGAGTCGGTGGG | | |
| | | CCTCGGGGGCGGGTGCGGGGTCGG | | |
| | | CGGGGCCGCCCCGGGTGGCTTCGG | | |
| | | TCGGAGCCATGGGGTCGTGCGCTC | | |
| | | CTTTCGGTCGGGCGCTGCGGGTCG | | |
| | | TGGGGCGGGCGTCAGGCACCGGGC | | |
| | | TTGCGGGTCATGCACCAGGTGCGC | | |
| | | GGTCCTTCGGGCACCTCGACGTCG | | |
| | | GCGGTGACGGTGAAGCCGAGCCGC | | |
| | | TCGTAGAAGGGGAGGTTGCGGGGC | | |
| | | GCGGAGGTCTCCAGGAAGGCGGG | | |
| | | CACCCCGGCGCGCTCGGCCGCCTC | | |
| | | CACTCCGGGGAGCACGACGGCGCT | | |
| | | GCCCAGACCCTTGCCCTGGTGGTC | | |
| | | GGGCGAGACGCCGACGGTGGCCA | | |
| | | GGAACCACGCGGGCTCCTTGGGCC | | |
| | | GGTGCGGCGCCAGGAGGCCTTCCA | | |
| | | TCTGTTGCTGCGCGGCCAGCCGGG | | |
| | | AACCGCTCAACTCGGCCATGCGCG | | |
| | | GGCCGATCTCGGCGAACACCGCCC | | |
| | | CCGCTTCGACGCTCTCCGGCGTGG | | |
| | | TCCAGACCGCCACCGCGGCGCCGT | | |
| | | CGTCCGCGACCCACACCTTGCCGA | | |
| | | TGTCGAGCCCGACGCGCGTGAGGA | | |
| | | AGAGTTCTTGCAGCTCGGTGACCC | | |
| | | GCTCGATGTGGCGGTCCGGGTCGA | | |
| | | CGGTGTGGCGCGTGGCGGGGTAGT | | |
| | | CGGCGAACGCGGCGGCGAGGGTG | | |
| | | CGTACGGCCCGGGGGACGTCGTCG | | |
| | | CGGGTGGCGAGGCGCACCGTGGGC | | |
| | | TTGTACTCGGTCATGGAAGGTCGT | | |
| | | CTCCTTGTGAGGGGTCAGGGGCGT | | |
| | | GGGTCAGGGGATGGTGGCGGCACC | | |
| | | GGTCGTGGCGGCCGACCTGCAGGC | | |
| | | ATGCAAGCTTTTTGCAAAAGCCTA | | |
| | | GGCCTCCAAAAAAGCCTCCTCACT | | |
| | | ACTTCTGGAATAGCTCAGAGGCCG | | |
| | | AGGCGGCCTCGGCCTCTGCATAAA | | |
| | | TAAAAAAAATTAGTCAGCCATGGG | | |
| | | GCGGAGAATGGGCGGAACTGGGC | | |
| | | GGAGTTAGGGGGGGGATGGGCGG | | |
| | | AGTTAGGGGCGGGACTATGGTTGC | | |
| | | TGACTAATTGAGATGCATGCTTTG | | |
| | | CATACTTCTGCCTGCTGGGGAGCC | | |
| | | TGGGGACTTTCCACACCTGGTTGC | | |
| | | TGACTAATTGAGATGCATGCTTTG | | |
| | | CATACTTCTGCCTGCTGGGGAGCC | | |
| | | TGGGGACTTTCCACACCCTAACTG | | |
| | | ACACACATTCCACAGAATTCAAGT | | |
| | | GATCTCCAAAAAATAAGTACTTTT | | |
| | | TGACTGTAAATAAAATTGTAAGGA | | |
| | | GTAAAAAGTACTTTTTTTTCTAAAA | | |
| | | AAATGTAATTAAGTAAAAGTAAAA | | |
| | | GTATTGATTTTTAATTGTACTCAAG | | |
| | | TAAAGTAAAAATCCCCAAAAATAA | | |
| | | TACTTAAGTACAGTAATCAAGTAA | | |
| | | AATTACTCAAGTACTTTACACCTCT | | |

TABLE 2
Sequences used in Example 2

| | SEQ | | | Included in | |
|---|---|---|---|---|---|
| Name of | ID | | | Optimized | |
| Construct | NO: | Sequence | Description | pegRNAs? | Notes |
| nick A | 410 | GTCCCAAAGTGCTGAGATTAT | nicking guide | | only spacer sequence |
| nick C | 411 | GTGTATTTTTTAGTAGATACT | nicking guide | Yes | only spacer sequence |
| nick G | 412 | GATTCTCCTGCCGCAGCCTC | nicking guide | | only spacer sequence |
| fxn spacer | 388 | GCAAGACTAACCTGGCCAACA | pegRNA for FXN, | Yes | only spacer sequence |
| f1 | | | spacer sequence | | |
| fxn spacer | 389 | GTCCGGAGTTCAAGACTAACC | pegRNA for FXN, | | only spacer sequence |
| f2 | | | spacer sequence | | |
| fxn spacer | 390 | GAAGGTGGATCACCTGAGGTC | pegRNA for FXN, | | only spacer sequence |
| f3 | | | spacer sequence | | |
| fxn spacer | 391 | GTCTGGAGTAGCTGGGATTAC | pegRNA for FXN, | | only spacer sequence |
| r1 | | | spacer sequence | | |
| fxn spacer | 392 | GCAGGCGCGCGACACCACGCC | pegRNA for FXN, | | only spacer sequence |
| r2 | | | spacer sequence | | |
| fxn_h32_pb | 816 | GCAAGACTAACCTGGCCAACAG | pegRNA for FXN, | | spacer + scaffold + flap |
| s8 | | TTTTAGAGCTAGAAATAGCAAG | 45 bp + 3*GAA | | |
| | | TTAAAATAAGGCTAGTCCGTTA | deletion | | |
| | | TCAACTTGAAAAAGTGGCACCG | | | |
| | | AGTCGGTGCATTACAGGCGCGC | | | |
| | | GACACCACGCCCGGCTAACTGG | | | |
| | | CCAGG | | | |
| fxn_h32_pb | 817 | GCAAGACTAACCTGGCCAACAG | pegRNA for FXN, | | spacer + scaffold + flap |
| s10 | | TTTTAGAGCTAGAAATAGCAAG | 45 bp + 3*GAA | | |
| | | TTAAAATAAGGCTAGTCCGTTA | deletion | | |
| | | TCAACTTGAAAAAGTGGCACCG | | | |
| | | AGTCGGTGCATTACAGGCGCGC | | | |
| | | GACACCACGCCCGGCTAACTGG | | | |
| | | CCAGGTT | | | |
| fxn_h32_pb | 818 | GCAAGACTAACCTGGCCAACAG | pegRNA for FXN, | | spacer + scaffold + flap |
| s12 | | TTTTAGAGCTAGAAATAGCAAG | 45 bp + 3*GAA | | |
| | | TTAAAATAAGGCTAGTCCGTTA | deletion | | |
| | | TCAACTTGAAAAAGTGGCACCG | | | |
| | | AGTCGGTGCATTACAGGCGCGC | | | |
| | | GACACCACGCCCGGCTAACTGG | | | |
| | | CCAGGTTAG | | | |
| fxn_h32_pb | 819 | GCAAGACTAACCTGGCCAACAG | pegRNA for FXN, | | spacer + scaffold + flap |
| s14 | | TTTTAGAGCTAGAAATAGCAAG | 45 bp + 3*GAA | | |
| | | TTAAAATAAGGCTAGTCCGTTA | deletion | | |
| | | TCAACTTGAAAAAGTGGCACCG | | | |
| | | AGTCGGTGCATTACAGGCGCGC | | | |
| | | GACACCACGCCCGGCTAAC | | | |
| | | TGGCCAGGTTAGTC | | | |
| fxn_h35_pb | 820 | GCAAGACTAACCTGGCCAACAG | pegRNA for FXN, | | spacer + scaffold + flap |
| s8 | | TTTTAGAGCTAGAAATAGCAAG | 45 bp + 3*GAA | | |
| | | TTAAAATAAGGCTAGTCCGTTA | deletion | | |
| | | TCAACTTGAAAAAGTGGCACCG | | | |
| | | AGTCGGTGCGGGATTACAGGCG | | | |
| | | CGCGACACCACGCCCGGCTAAC | | | |
| | | TGGCCAGG | | | |
| fxn_h35_pb | 821 | GCAAGACTAACCTGGCCAACAG | pegRNA for FXN, | | spacer + scaffold + flap |
| s10 | | TTTTAGAGCTAGAAATAGCAAG | 45 bp + 3*GAA | | |
| | | TTAAAATAAGGCTAGTCCGTTA | deletion | | |
| | | TCAACTTGAAAAAGTGGCACCG | | | |
| | | AGTCGGTGCGGGATTACAGGCG | | | |
| | | CGCGACACCACGCCCGGCTAAC | | | |
| | | TGGCCAGGTT | | | |
| fxn_h35_pb | 822 | GCAAGACTAACCTGGCCAACAG | pegRNA for FXN, | | spacer + scaffold + flap |
| s12 | | TTTTAGAGCTAGAAATAGCAAG | 45 bp + 3*GAA | | |
| | | TTAAAATAAGGCTAGTCCGTTA | deletion | | |
| | | TCAACTTGAAAAAGTGGCACCG | | | |
| | | AGTCGGTGCGGGATTACAGGCG | | | |
| | | CGCGACACCACGCCCGGCTAAC | | | |
| | | TGGCCAGGTTAG | | | |
| fxn_h35_pb | 823 | GCAAGACTAACCTGGCCAACAG | pegRNA for FXN, | | spacer + scaffold + flap |
| s14 | | TTTTAGAGCTAGAAATAGCAAG | 45 bp + 3*GAA | | |
| | | TTAAAATAAGGCTAGTCCGTTA | deletion | | |
| | | TCAACTTGAAAAAGTGGCACCG | | | |
| | | AGTCGGTGCGGGATTACAGGCG | | | |
| | | CGCGACACCACGCCCGGCTAAC | | | |
| | | TGGCCAGGTTAGTC | | | |
| fxn_h40_pb | 824 | GCAAGACTAACCTGGCCAACAG | pegRNA for FXN, | | spacer + scaffold + flap |
| s8 | | TTTTAGAGCTAGAAATAGCAAG | 45 bp + 3*GAA | | |
| | | TTAAAATAAGGCTAGTCCGTTA | deletion | | |
| | | TCAACTTGAAAAAGTGGCACCG | | | |
| | | AGTCGGTGCTAGCTGGGATTAC | | | |
| | | AGGCGCGCGACACCACGCCCG | | | |
| | | GCTAACTGGCCAGG | | | |
| fxn_h40_pb | 825 | GCAAGACTAACCTGGCCAACAG | pegRNA for FXN, | | spacer + scaffold + flap |
| s10 | | TTTTAGAGCTAGAAATAGCAAG | 45 bp + 3*GAA | | |
| | | TTAAAATAAGGCTAGTCCGTTA | | | |
| | | TCAACTTGAAAAAGTGGCACCG | deletion | | |
| | | AGTCGGTGCTAGCTGGGATTAC | | | |
| | | AGGCGCGCGACACCACGCCCG | | | |
| | | GCTAACTGGCCAGGTT | | | |
| fxn_h40_pb | 826 | GCAAGACTAACCTGGCCAACAG | pegRNA for FXN, | | spacer + scaffold + flap |
| s12 | | TTTTAGAGCTAGAAATAGCAAG | 45 bp + 3*GAA | | |
| | | TTAAAATAAGGCTAGTCCGTTA | deletion | | |
| | | TCAACTTGAAAAAGTGGCACCG | | | |
| | | AGTCGGTGCTAGCTGGGATTAC | | | |
| | | AGGCGCGCGACACCACGCCCG | | | |
| | | GCTAACTGGCCAGGTTAG | | | |
| fxn_h40_pb | 827 | GCAAGACTAACCTGGCCAACAG | pegRNA for FXN, | | spacer + scaffold + flap |
| s14 | | TTTTAGAGCTAGAAATAGCAAG | 45 bp + 3*GAA | | |
| | | TTAAAATAAGGCTAGTCCGTTA | deletion | | |
| | | TCAACTTGAAAAAGTGGCACCG | | | |
| | | AGTCGGTGCTAGCTGGGATTAC | | | |
| | | AGGCGCGCGACACCACGCCCG | | | |
| | | GCTAACTGGCCAGGTTAGTC | | | |
| fxn_h40_pb | 828 | GCAAGACTAACCTGGCCAACAG | epegRNA with | | spacer + scaffold + flap + |
| s8eq1 | | TTTTAGAGCTAGAAATAGCAAG | rational linker, | | linker + 3′motif |
| | | TTAAAATAAGGCTAGTCCGTTA | 45 bp + 3*GAA | | |
| | | TCAACTTGAAAAAGTGGCACCG | deletion | | |
| | | AGTCGGTGCTAGCTGGGATTAC | | | |
| | | AGGCGCGCGACACCACGCCCG | | | |
| | | GCTAACTGGCCAGGCAATCACT | | | |
| | | CGCGGTTCTATCTAGTTACGCG | | | |
| | | TTAAACCAACTAGAA | | | |
| fxn_h40_pb | 829 | GCAAGACTAACCTGGCCAACAG | epegRNA with | Yes | spacer + scaffold + flap + |
| s10eq1 | | TTTTAGAGCTAGAAATAGCAAG | rational linker, | | linker + 3′motif |
| | | TTAAAATAAGGCTAGTCCGTTA | 45 bp + 3*GAA | | |
| | | TCAACTTGAAAAAGTGGCACCG | deletion | | |
| | | AGTCGGTGCTAGCTGGGATTAC | | | |
| | | AGGCGCGCGACACCACGCCCG | | | |
| | | GCTAACTGGCCAGGTTCAATCA | | | |
| | | CTCGCGGTTCTATCTAGTTACG | | | |
| | | CGTTAAACCAACTAGAA | | | |
| fxn_h40_pb | 830 | GCAAGACTAACCTGGCCAACAG | epegRNA with | | spacer + scaffold + flap + |
| s12eq1 | | TTTTAGAGCTAGAAATAGCAAG | rational linker, | | linker + 3′motif |
| | | TTAAAATAAGGCTAGTCCGTTA | 45 bp + 3*GAA | | |
| | | TCAACTTGAAAAAGTGGCACCG | deletion | | |
| | | AGTCGGTGCTAGCTGGGATTAC | | | |
| | | AGGCGCGCGACACCACGCCCG | | | |
| | | GCTAACTGGCCAGGTTAGCAAT | | | |
| | | CACTCGCGGTTCTATCTAGTTA | | | |
| | | CGCGTTAAACCAACTAGAA | | | |
| fxn_h40_pb | 831 | GCAAGACTAACCTGGCCAACAG | epegRNA with | | spacer + scaffold + flap + |
| s14eq1 | | TTTTAGAGCTAGAAATAGCAAG | rational linker, | | linker + 3′motif |
| | | TTAAAATAAGGCTAGTCCGTTA | 45 bp + 3*GAA | | |
| | | TCAACTTGAAAAAGTGGCACCG | deletion | | |
| | | AGTCGGTGCTAGCTGGGATTAC | | | |
| | | AGGCGCGCGACACCACGCCCG | | | |
| | | GCTAACTGGCCAGGTTAGTCCA | | | |
| | | ATCACTCGCGGTTCTATCTAGTT | | | |
| | | ACGCGTTAAACCAACTAGAA | | | |
| fxn_h40_pb | 832 | GCAAGACTAACCTGGCCAACAG | epegRNA with | | spacer + scaffold + flap + |
| s8eq1A | | TTTTAGAGCTAGAAATAGCAAG | pegLIT linker, 45 bp | | linker + 3′motif |
| | | TTAAAATAAGGCTAGTCCGTTA | + 3*GAA deletion | | |
| | | TCAACTTGAAAAAGTGGCACCG | | | |
| | | AGTCGGTGCTAGCTGGGATTAC | | | |
| | | AGGCGCGCGACACCACGCCCG | | | |
| | | GCTAACTGGCCAGGCAAATAAC | | | |
| | | CGCGGTTCTATCTAGTTACGCG | | | |
| | | TTAAACCAACTAGAA | | | |
| fxn_h40_pb | 833 | GCAAGACTAACCTGGCCAACAG | epegRNA with | Yes | spacer + scaffold + flap + |
| s10eq1A | | TTTTAGAGCTAGAAATAGCAAG | pegLIT linker, 45 bp | | linker + 3′motif |
| | | TTAAAATAAGGCTAGTCCGTTA | + 3*GAA deletion | | |
| | | TCAACTTGAAAAAGTGGCACCG | | | |
| | | AGTCGGTGCTAGCTGGGATTAC | | | |
| | | AGGCGCGCGACACCACGCCCG | | | |
| | | GCTAACTGGCCAGGTTCAAATA | | | |
| | | ACCGCGGTTCTATCTAGTTACG | | | |
| | | CGTTAAACCAACTAGAA | | | |
| fxn_h40_pb | 834 | GCAAGACTAACCTGGCCAACAG | epegRNA with | | spacer + scaffold + flap + |
| s12eq1A | | TTTTAGAGCTAGAAATAGCAAG | pegLIT linker, 45 bp | | linker + 3′motif |
| | | TTAAAATAAGGCTAGTCCGTTA | + 3*GAA deletion | | |
| | | TCAACTTGAAAAAGTGGCACCG | | | |
| | | AGTCGGTGCTAGCTGGGATTAC | | | |
| | | AGGCGCGCGACACCACGCCCG | | | |
| | | GCTAACTGGCCAGGTTAGCAAA | | | |
| | | TAACCGCGGTTCTATCTAGTTA | | | |
| | | CGCGTTAAACCAACTAGAA | | | |
| fxn_h40_pb | 835 | GCAAGACTAACCTGGCCAACAG | epegRNA with | | spacer + scaffold + flap + |
| s14eq1A | | TTTTAGAGCTAGAAATAGCAAG | pegLIT linker, 45 bp | | linker + 3′motif |
| | | TTAAAATAAGGCTAGTCCGTTA | + 3*GAA deletion | | |
| | | TCAACTTGAAAAAGTGGCACCG | | | |
| | | AGTCGGTGCTAGCTGGGATTAC | | | |
| | | AGGCGCGCGACACCACGCCCG | | | |
| | | GCTAACTGGCCAGGTTAGTCCA | | | |
| | | AATAACCGCGGTTCTATCTAGT | | | |
| | | TACGCGTTAAACCAACTAGAA | | | |
| fxn_Pdel_f1_ | 836 | GCAAGACTAACCTGGCCAACAG | epegRNA for dual | | spacer + scaffold + flap + |
| r1_p8eq1 | | TTTTAGAGCTAGAAATAGCAAG | pegRNA approach, | | linker + 3′motif |
| | | TTAAAATAAGGCTAGTCCGTTA | 45 bp + 3*GAA | | |
| | | TCAACTTGAAAAAGTGGCACCG | deletion | | |
| | | AGTCGGTGCTCCTGCCGCAGCC | | | |
| | | TCTGGAGTAGCTGGGATTGGCC | | | |
| | | AGGCAAATAACCGCGGTTCTAT | | | |
| | | CTAGTTACGCGTTAAACCAACT | | | |
| | | AGAA | | | |
| fxn_Pdel_r1_ | 837 | GTCTGGAGTAGCTGGGATTACG | epegRNA for dual | | spacer + scaffold + flap + |
| f1_p12eq1 | | TTTTAGAGCTAGAAATAGCAAG | pegRNA approach, | | linker + 3′motif |
| | | TTAAAATAAGGCTAGTCCGTTA | 45 bp + 3*GAA | | |
| | | TCAACTTGAAAAAGTGGCACCG | deletion | | |
| | | AGTCGGTGCTGAGGTCCGGAGT | | | |
| | | TCAAGACTAACCTGGCCAATCC | | | |
| | | CAGCTACTCAAATAACCGCGGT | | | |
| | | TCTATCTAGTTACGCGTTAAAC | | | |
| | | CAACTAGAA | | | |
| fxn_Pdel_f1_ | 838 | GCAAGACTAACCTGGCCAACAG | epegRNA for dual | | spacer + scaffold + flap + |
| r2_p12eq1 | | TTTTAGAGCTAGAAATAGCAAG | pegRNA approach, | | linker + 3′motif |
| | | TTAAAATAAGGCTAGTCCGTTA | 45 bp + 3*GAA | | |
| | | TCAACTTGAAAAAGTGGCACCG | deletion | | |
| | | AGTCGGTGCAGTAGCTGGGATT | | | |
| | | ACAGGCGCGCGACACCACTGGC | | | |
| | | CAGGTTAGCAAATAACCGCGGT | | | |
| | | TCTATCTAGTTACGCGTTAAAC | | | |
| | | CAACTAGAA | | | |
| fxn_Pdel_r2_ | 839 | GCAGGCGCGCGACACCACGCC | epegRNA for dual | | spacer + scaffold + flap + |
| f1_p10eq1 | | GTTTTAGAGCTAGAAATAGCAA | pegRNA approach, | | linker + 3′motif |
| | | GTTAAAATAAGGCTAGTCCGTT | 45 bp + 3*GAA | | |
| | | ATCAACTTGAAAAAGTGGCACC | deletion | | |
| | | GAGTCGGTGCTGAGGTCCGGAG | | | |
| | | TTCAAGACTAACCTGGCCAGTG | | | |
| | | GTGTCGCCAAATAACCGCGGTT | | | |
| | | CTATCTAGTTACGCGTTAAACC | | | |
| | | AACTAGAA | | | |
| fxn_dflapH | 840 | GCAAGACTAACCTGGCCAACAG | epegRNA for dual | | spacer + scaffold + flap + |
| A_f1_r1_p8 | | TTTTAGAGCTAGAAATAGCAAG | pegRNA approach, | | linker + 3′motif |
| eq1 | | TTAAAATAAGGCTAGTCCGTTA | 45 bp + 3*GAA | | |
| | | TCAACTTGAAAAAGTGGCACCG | deletion | | |
| | | AGTCGGTGCTACAGGCGCGCGA | | | |
| | | CACCACATACTGGGTTTCACCA | | | |
| | | TGTTGGCCAGGCAAATAACCGC | | | |
| | | GGTTCTATCTAGTTACGCGTTA | | | |
| | | AACCAACTAGAA | | | |
| fxn_dflapH | 841 | GCAAGACTAACCTGGCCAACAG | epegRNA for dual | | spacer + scaffold + flap + |
| A_f1_r2_p1 | | TTTTAGAGCTAGAAATAGCAAG | pegRNA approach, | | linker + 3′motif |
| 0eq1 | | TTAAAATAAGGCTAGTCCGTTA | 45 bp + 3*GAA | | |
| | | TCAACTTGAAAAAGTGGCACCG | deletion | | |
| | | AGTCGGTGCTAGATACTGGGTT | | | |
| | | TCACCATGTTGGCCAGGTTCAA | | | |
| | | ATAACCGCGGTTCTATCTAGTT | | | |
| | | ACGCGTTAAACCAACTAGAA | | | |
| fxn_dflapH | 842 | GTCTGGAGTAGCTGGGATTACG | epegRNA for dual | | spacer + scaffold + flap |
| A_r1_f1_p1 | | TTTTAGAGCTAGAAATAGCAAG | pegRNA approach, | | linker + 3′motif |
| 2eq1 | | TTAAAATAAGGCTAGTCCGTTA | 45 bp + 3*GAA | | |
| | | TCAACTTGAAAAAGTGGCACCG | deletion | | |
| | | AGTCGGTGCACATGGTGAAACC | | | |
| | | CAGTATGTGGTGTCGCGCGCCT | | | |
| | | GTAATCCCAGCTACTCAAATAA | | | |
| | | CCGCGGTTCTATCTAGTTACGC | | | |
| | | GTTAAACCAACTAGAA | | | |
| fxn_dflapH | 843 | GCAGGCGCGCGACACCACGCC | epegRNA for dual | | spacer + scaffold + flap + |
| A_r2_f1_p1 | | GTTTTAGAGCTAGAAATAGCAA | pegRNA approach, | | linker + 3′motif |
| 2eq1 | | GTTAAAATAAGGCTAGTCCGTT | 45 bp + 3*GAA | | |
| | | ATCAACTTGAAAAAGTGGCACC | deletion | | |
| | | GAGTCGGTGCACATGGTGAAAC | | | |
| | | CCAGTATCTAGTGGTGTCGCGC | | | |
| | | CAAATAACCGCGGTTCTATCTA | | | |
| | | GTTACGCGTTAAACCAACTAGA | | | |
| | | A | | | |
| fxn_dflapS | 844 | GCAAGACTAACCTGGCCAACAG | epegRNA for dual | | spacer + scaffold + flap |
| A_f1_r1_p1 | | TTTTAGAGCTAGAAATAGCAAG | pegRNA approach, | | linker + 3′motif |
| 0eq1 | | TTAAAATAAGGCTAGTCCGTTA | 45 bp + 3*GAA | | |
| | | TCAACTTGAAAAAGTGGCACCG | deletion | | |
| | | AGTCGGTGCTACAGGCGCGCGA | | | |
| | | CACCACGCCTGGCCAGGTTCAA | | | |
| | | ATAACCGCGGTTCTATCTAGTT | | | |
| | | ACGCGTTAAACCAACTAGAA | | | |
| fxn_dflapS | 845 | GCAAGACTAACCTGGCCAACAG | epegRNA for dual | | spacer + scaffold + flap + |
| A_f1_r2_p1 | | TTTTAGAGCTAGAAATAGCAAG | pegRNA approach, | | linker + 3′motif |
| 2eq1 | | TTAAAATAAGGCTAGTCCGTTA | 45 bp + 3*GAA | | |
| | | TCAACTTGAAAAAGTGGCACCG | deletion | | |
| | | AGTCGGTGCGCCCGGCTAACTT | | | |
| | | TTCTTTATGGCCAGGTTAGCAA | | | |
| | | ATAACCGCGGTTCTATCTAGTT | | | |
| | | ACGCGTTAAACCAACTAGAA | | | |
| fxn_dflapS | 846 | GCAAGACTAACCTGGCCAACAG | epegRNA for dual | | spacer + scaffold + flap + |
| A_r1_f1_p1 | | TTTTAGAGCTAGAAATAGCAAG | pegRNA approach, | | linker + 3′motif |
| 0eq1 | | TTAAAATAAGGCTAGTCCGTTA | 45 bp + 3*GAA | | |
| | | TCAACTTGAAAAAGTGGCACCG | deletion | | |
| | | AGTCGGTGCACATGGTGAAACC | | | |
| | | CAGTATCTAATCCCAGCTACAA | | | |
| | | ATAACCGCGGTTCTATCTAGTT | | | |
| | | ACGCGTTAAACCAACTAGAA | | | |
| fxn_dflapS | 847 | GCAAGACTAACCTGGCCAACAG | epegRNA for dual | | spacer + scaffold + flap + |
| A_r2_f1_p1 | | TTTTAGAGCTAGAAATAGCAAG | pegRNA approach, | | linker + 3′motif |
| 0eq1 | | TTAAAATAAGGCTAGTCCGTTA | 45 bp + 3*GAA | | |
| | | TCAACTTGAAAAAGTGGCACCG | deletion | | |
| | | AGTCGGTGCACATGGTGAAACC | | | |
| | | CAGTATCTAGTGGTGTCGCCAA | | | |
| | | ATAACCGCGGTTCTATCTAGTT | | | |
| | | ACGCGTTAAACCAACTAGAA | | | |
| fxn_Pdel_f1_ | 848 | GCAAGACTAACCTGGCCAACAG | pegRNA for dual | | spacer + scaffold + flap |
| r1_p8 | | TTTTAGAGCTAGAAATAGCAAG | pegRNA approach, | | |
| | | TTAAAATAAGGCTAGTCCGTTA | 45 bp + 3*GAA | | |
| | | TCAACTTGAAAAAGTGGCACCG | deletion | | |
| | | AGTCGGTGCTCCTGCCGCAGCC | | | |
| | | TCTGGAGTAGCTGGGATTGGCC | | | |
| | | AGG | | | |
| fxn_Pdel_r1_ | 849 | GTCTGGAGTAGCTGGGATTACG | pegRNA for dual | | spacer + scaffold + flap |
| f1_p12 | | TTTTAGAGCTAGAAATAGCAAG | pegRNA approach, | | |
| | | TTAAAATAAGGCTAGTCCGTTA | 45 bp + 3*GAA | | |
| | | TCAACTTGAAAAAGTGGCACCG | deletion | | |
| | | AGTCGGTGCTGAGGTCCGGAGT | | | |
| | | TCAAGACTAACCTGGCCAATCC | | | |
| | | CAGCTACT | | | |
| fxn_Pdel_f1_ | 850 | GCAAGACTAACCTGGCCAACAG | pegRNA for dual | | spacer + scaffold + flap |
| r2_p12 | | TTTTAGAGCTAGAAATAGCAAG | pegRNA approach, | | |
| | | TTAAAATAAGGCTAGTCCGTTA | 45 bp + 3*GAA | | |
| | | TCAACTTGAAAAAGTGGCACCG | deletion | | |
| | | AGTCGGTGCAGTAGCTGGGATT | | | |
| | | ACAGGCGCGCGACACCACTGGC | | | |
| | | CAGGTTAG | | | |
| fxn_Pdel_r2_ | 851 | GCAGGCGCGCGACACCACGCC | pegRNA for dual | | spacer + scaffold + flap |
| f1_p10 | | GTTTTAGAGCTAGAAATAGCAA | pegRNA approach, | | |
| | | GTTAAAATAAGGCTAGTCCGTT | 45 bp + 3*GAA | | |
| | | ATCAACTTGAAAAAGTGGCACC | deletion | | |
| | | GAGTCGGTGCTGAGGTCCGGAG | | | |
| | | TTCAAGACTAACCTGGCCAGTG | | | |
| | | GTGTCGC | | | |
| fxn_dflapH | 852 | GCAAGACTAACCTGGCCAACAG | pegRNA for dual | | spacer + scaffold + flap |
| A_f1_r1_p8 | | TTTTAGAGCTAGAAATAGCAAG | pegRNA approach, | | |
| | | TTAAAATAAGGCTAGTCCGTTA | 45 bp + 3*GAA | | |
| | | TCAACTTGAAAAAGTGGCACCG | deletion | | |
| | | AGTCGGTGCTACAGGCGCGCGA | | | |
| | | CACCACATACTGGGTTTCACCA | | | |
| | | TGTTGGCCAGG | | | |
| fxn_dflapH | 853 | GCAAGACTAACCTGGCCAACAG | pegRNA for dual | | spacer + scaffold + flap |
| A_f1_r2_p1 | | TTTTAGAGCTAGAAATAGCAAG | pegRNA approach, | | |
| 0 | | TTAAAATAAGGCTAGTCCGTTA | 45 bp + 3*GAA | | |
| | | TCAACTTGAAAAAGTGGCACCG | deletion | | |
| | | AGTCGGTGCTAGATACTGGGTT | | | |
| | | TCACCATGTTGGCCAGGTT | | | |
| fxn_dflapH | 854 | GTCTGGAGTAGCTGGGATTACG | pegRNA for dual | | spacer + scaffold + flap |
| A_r1_f1_p1 | | TTTTAGAGCTAGAAATAGCAAG | pegRNA approach, | | |
| 2 | | TTAAAATAAGGCTAGTCCGTTA | 45 bp + 3*GAA | | |
| | | TCAACTTGAAAAAGTGGCACCG | deletion | | |
| | | AGTCGGTGCACATGGTGAAACC | | | |
| | | CAGTATGTGGTGTCGCGCGCCT | | | |
| | | GTAATCCCAGCTACT | | | |
| fxn_dflapH | 855 | GCAGGCGCGCGACACCACGCC | pegRNA for dual | | spacer + scaffold + flap |
| A_r2_f1_p1 | | GTTTTAGAGCTAGAAATAGCAA | pegRNA approach, | | |
| 2 | | GTTAAAATAAGGCTAGTCCGTT | 45 bp + 3*GAA | | |
| | | ATCAACTTGAAAAAGTGGCACC | deletion | | |
| | | GAGTCGGTGCACATGGTGAAAC | | | |
| | | CCAGTATCTAGTGGTGTCGCGC | | | |
| fxn_dflapS | 856 | GCAAGACTAACCTGGCCAACAG | pegRNA for dual | | spacer + scaffold + flap |
| A_f1_r1_p1 | | TTTTAGAGCTAGAAATAGCAAG | pegRNA approach, | | |
| 0 | | TTAAAATAAGGCTAGTCCGTTA | 45 bp + 3*GAA | | |
| | | TCAACTTGAAAAAGTGGCACCG | deletion | | |
| | | AGTCGGTGCTACAGGCGCGCGA | | | |
| | | CACCACGCCTGGCCAGGTT | | | |
| fxn_dflapS | 857 | GCAAGACTAACCTGGCCAACAG | pegRNA for dual | | spacer + scaffold + flap |
| A_f1_r2_p1 | | TTTTAGAGCTAGAAATAGCAAG | pegRNA approach, | | |
| 2 | | TTAAAATAAGGCTAGTCCGTTA | 45 bp + 3*GAA | | |
| | | TCAACTTGAAAAAGTGGCACCG | deletion | | |
| | | AGTCGGTGCGCCCGGCTAACTT | | | |
| | | TTCTTTATGGCCAGGTTAG | | | |
| fxn_dflapS | 858 | GCAAGACTAACCTGGCCAACAG | pegRNA for dual | | spacer + scaffold + flap |
| A_r1_f1_p1 | | TTTTAGAGCTAGAAATAGCAAG | pegRNA approach, | | |
| 0 | | TTAAAATAAGGCTAGTCCGTTA | 45 bp + 3*GAA | | |
| | | TCAACTTGAAAAAGTGGCACCG | deletion | | |
| | | AGTCGGTGCACATGGTGAAACC | | | |
| | | CAGTATCTAATCCCAGCTA | | | |
| fxn_dflapS | 859 | GCAAGACTAACCTGGCCAACAG | pegRNA for dual | | spacer + scaffold + flap |
| A_r2_f1_p1 | | TTTTAGAGCTAGAAATAGCAAG | pegRNA approach, | | |
| 0 | | TTAAAATAAGGCTAGTCCGTTA | 45 bp + 3*GAA | | |
| | | TCAACTTGAAAAAGTGGCACCG | deletion | | |
| | | AGTCGGTGCACATGGTGAAACC | | | |
| | | CAGTATCTAGTGGTGTCGC | | | |
| nick PE3b | 413 | GCGACACCACGCCCGGCTAAC | nicking guide | Yes | only spacer sequence |
| fxn_h40_pb | 860 | GCAAGACTAACCTGGCCAACAG | epegRNA with | | spacer + scaffold + flap + |
| s8UA_eq1 | | TTTAAGAGCTAGAAATAGCAAG | pegLIT linker, | | linker + 3′motif |
| A | | TTTAAATAAGGCTAGTCCGTTA | scaffold UAflip, | | |
| | | TCAACTTGAAAAAGTGGCACCG | 45 bp + 3*GAA | | |
| | | AGTCGGTGCTAGCTGGGATTAC | deletion | | |
| | | AGGCGCGCGACACCACGCCCG | | | |
| | | GCTAACTGGCCAGGCAAATAAC | | | |
| | | CGCGGTTCTATCTAGTTACGCG | | | |
| | | TTAAACCAACTAGAA | | | |
| fxn_h40_pb | 861 | GCAAGACTAACCTGGCCAACAG | epegRNA with | Yes | spacer + scaffold + flap + |
| s10UA_eq1 | | TTTAAGAGCTAGAAATAGCAAG | pegLIT linker, | | linker + 3′motif |
| A | | TTTAAATAAGGCTAGTCCGTTA | scaffold UAflip, | | |
| | | TCAACTTGAAAAAGTGGCACCG | 45 bp + 3*GAA | | |
| | | AGTCGGTGCTAGCTGGGATTAC | deletion | | |
| | | AGGCGCGCGACACCACGCCCG | | | |
| | | GCTAACTGGCCAGGTTCAAATA | | | |
| | | ACCGCGGTTCTATCTAGTTACG | | | |
| | | CGTTAAACCAACTAGAA | | | |
| fxn_h40_pb | 862 | GCAAGACTAACCTGGCCAACAG | epegRNA with | | spacer + scaffold + flap + |
| s12UA_eq1 | | TTTAAGAGCTAGAAATAGCAAG | pegLIT linker, | | linker + 3′motif |
| A | | TTTAAATAAGGCTAGTCCGTTA | scaffold UAflip, | | |
| | | TCAACTTGAAAAAGTGGCACCG | 45 bp + 3*GAA | | |
| | | AGTCGGTGCTAGCTGGGATTAC | deletion | | |
| | | AGGCGCGCGACACCACGCCCG | | | |
| | | GCTAACTGGCCAGGTTAGCAAA | | | |
| | | TAACCGCGGTTCTATCTAGTTA | | | |
| | | CGCGTTAAACCAACTAGAA | | | |
| fxn_h40_pb | 863 | GCAAGACTAACCTGGCCAACAG | epegRNA with | | spacer + scaffold + flap + |
| s14UA_eq1 | | TTTAAGAGCTAGAAATAGCAAG | pegLIT linker, | | linker + 3′motif |
| A | | TTTAAATAAGGCTAGTCCGTTA | scaffold UAflip, | | |
| | | TCAACTTGAAAAAGTGGCACCG | 45 bp + 3*GAA | | |
| | | AGTCGGTGCTAGCTGGGATTAC | deletion | | |
| | | AGGCGCGCGACACCACGCCCG | | | |
| | | GCTAACTGGCCAGGTTAGTCCA | | | |
| | | AATAACCGCGGTTCTATCTAGT | | | |
| | | TACGCGTTAAACCAACTAGAA | | | |
| fxn_h40_pb | 864 | GCAAGACTAACCTGGCCAACAG | epegRNA with | | spacer + scaffold + flap + |
| s8UA | | TTTAAGAGCTAGAAATAGCAAG | scaffold UAflip, | | linker + 3′motif |
| | | TTTAAATAAGGCTAGTCCGTTA | 45 bp + 3*GAA | | |
| | | TCAACTTGAAAAAGTGGCACCG | deletion | | |
| | | AGTCGGTGCTAGCTGGGATTAC | | | |
| | | AGGCGCGCGACACCACGCCCG | | | |
| | | GCTAACTGGCCAGG | | | |
| fxn_h40_pb | 865 | GCAAGACTAACCTGGCCAACAG | epegRNA with | | spacer + scaffold + flap + |
| s10UA | | TTTAAGAGCTAGAAATAGCAAG | scaffold UAflip, | | linker + 3′motif |
| | | TTTAAATAAGGCTAGTCCGTTA | 45 bp + 3*GAA | | |
| | | TCAACTTGAAAAAGTGGCACCG | deletion | | |
| | | AGTCGGTGCTAGCTGGGATTAC | | | |
| | | AGGCGCGCGACACCACGCCCG | | | |
| | | GCTAACTGGCCAGGTT | | | |
| fxn_h40_pb | 866 | GCAAGACTAACCTGGCCAACAG | epegRNA with | | spacer + scaffold + flap + |
| s12UA | | TTTAAGAGCTAGAAATAGCAAG | scaffold UAflip, | | linker + 3′motif |
| | | TTTAAATAAGGCTAGTCCGTTA | 45 bp + 3*GAA | | |
| | | TCAACTTGAAAAAGTGGCACCG | deletion | | |
| | | AGTCGGTGCTAGCTGGGATTAC | | | |
| | | AGGCGCGCGACACCACGCCCG | | | |
| | | GCTAACTGGCCAGGTTAG | | | |
| fxn_h40_pb | 867 | GCAAGACTAACCTGGCCAACAG | epegRNA with | | spacer + scaffold + flap + |
| s14UA | | TTTAAGAGCTAGAAATAGCAAG | scaffold UAflip, | | linker + 3′motif |
| | | TTTAAATAAGGCTAGTCCGTTA | 45 bp + 3*GAA | | |
| | | TCAACTTGAAAAAGTGGCACCG | deletion | | |
| | | AGTCGGTGCTAGCTGGGATTAC | | | |
| | | AGGCGCGCGACACCACGCCCG | | | |
| | | GCTAACTGGCCAGGTTAGTC | | | |
| FXN | 872 | GGTACCGGCATATGGTTCTTGA | contruct integrated | Yes | Tol 3'-promoter- |
| intron 1 | | CAGAGGTGTAAAAAGTACTCAA | into mESCs for cell | | FXN fragment_p2A- |
| 30GAA | | AAATTTTACTCAAGTGAAAGTA | line generation | | GFP-puroR-Tol 5′ |
| fragment | | CAAGTACTTAGGGAAAATTTTA | | | |
| | | CTCAATTAAAAGTAAAAGTATC | | | |
| | | TGGCTAGAATCTTACTTGAGTA | | | |
| | | AAAGTAAAAAAGTACTCCATTA | | | |
| | | AAATTGTACTTGAGTATTAAGG | | | |
| | | AAGTAAAAGTAAAAGCAAGAA | | | |
| | | AGATCGATCTCGAAGGATCTGG | | | |
| | | AGGCCACCATGGTGTCGATAAC | | | |
| | | TTCGTATAGCATACATTATACG | | | |
| | | AAGTTATCGTGCTCGACATTGA | | | |
| | | TTATTGACTAGTTATTAATAGT | | | |
| | | AATCAATTACGGGGTCATTAGT | | | |
| | | TCATAGCCCATATATGGAGTTC | | | |
| | | CGCGTTACATAACTTACGGTAA | | | |
| | | ATGGCCCGCCTGGCTGACCGCC | | | |
| | | CAACGACCCCCGCCCATTGACG | | | |
| | | TCAATAATGACGTATGTTCCCA | | | |
| | | TAGTAACGCCAATAGGGACTTT | | | |
| | | CCATTGACGTCAATGGGTGGAG | | | |
| | | TATTTACGGTAAACTGCCCACT | | | |
| | | TGGCAGTACATCAAGTGTATCA | | | |
| | | TATGCCAAGTACGCCCCCTATT | | | |
| | | GACGTCAATGACGGTAAATGGC | | | |
| | | CCGCCTGGCATTATGCCCAGTA | | | |
| | | CATGACCTTATGGGACTTTCCT | | | |
| | | ACTTGGCAGTACATCTACGTAT | | | |
| | | TAGTCATCGCTATTACCATGGT | | | |
| | | CGAGGTGAGCCCCACGTTCTGC | | | |
| | | TTCACTCTCCCCATCTCCCCCCC | | | |
| | | CTCCCCACCCCCAATTTTGTATT | | | |
| | | TATTTATTTTTTAATTATTTTGT | | | |
| | | GCAGCGATGGGGGCGGGGGGG | | | |
| | | GGGGGGGGGCGCGCGCCAGGC | | | |
| | | GGGGCGGGGCGGGGCGAGGGG | | | |
| | | CGGGGCGGGGCGAGGCGGAGA | | | |
| | | GGTGCGGCGGCAGCCAATCAG | | | |
| | | AGCGGCGCGCTCCGAAAGTTTC | | | |
| | | CTTTTATGGCGAGGCGGCGGCG | | | |
| | | GCGGCGGCCCTATAAAAAGCG | | | |
| | | AAGCGCGCGGCGGGCGGGGAG | | | |
| | | TCGCTGCGACGCTGCCTTCGCC | | | |
| | | CCGTGCCCCGCTCCGCCGCCGC | | | |
| | | CTCGCGCCGCCCGCCCCGGCTC | | | |
| | | TGACTGACCGCGTTACTCCCAC | | | |
| | | AGGTGAGCGGGCGGGACGGCC | | | |
| | | CTTCTCCTCCGGGCTGTAATTA | | | |
| | | GCGCTTGGTTTAATGACGGCTT | | | |
| | | GTTTCTTTTCTGTGGCTGCGTGA | | | |
| | | AAGCCTTGAGGGGCTCCGGGAG | | | |
| | | GGCCCTTTGTGCGGGGGGAGCG | | | |
| | | GCTCGGGGGGTGCGTGCGTGTG | | | |
| | | TGTGTGCGTGGGGAGCGCCGCG | | | |
| | | TGCGGCTCCGCGCTGCCCGGCG | | | |
| | | GCTGTGAGCGCTGCGGGCGCGG | | | |
| | | CGCGGGGCTTTGTGCGCTCCGC | | | |
| | | AGTGTGCGCGAGGGGAGCGCG | | | |
| | | GCCGGGGGCGGTGCCCCGCGGT | | | |
| | | GCGGGGGGGGCTGCGAGGGGA | | | |
| | | ACAAAGGCTGCGTGCGGGGTGT | | | |
| | | GTGCGTGGGGGGGTGAGCAGG | | | |
| | | GGGTGTGGGCGCGTCGGTCGGG | | | |
| | | CTGCAACCCCCCCTGCACCCCC | | | |
| | | CTCCCCGAGTTGCTGAGCACGG | | | |
| | | CCCGGCTTCGGGTGCGGGGCTC | | | |
| | | CGTACGGGGCGTGGCGCGGGG | | | |
| | | CTCGCCGTGCCGGGCGGGGGGT | | | |
| | | GGCGGCAGGTGGGGGTGCCGG | | | |
| | | GCGGGGGGGGCCGCCTCGGG | | | |
| | | CCGGGGAGGGCTCGGGGGAGG | | | |
| | | GGCGCGGCGGCCCCCGGAGCG | | | |
| | | CCGGCGGCTGTCGAGGCGCGGC | | | |
| | | GAGCCGCAGCCATTGCCTTTTA | | | |
| | | TGGTAATCGTGCGAGAGGGCGC | | | |
| | | AGGGACTTCCTTTGTCCCAAAT | | | |
| | | CTGTGCGGAGCCGAAATCTGGG | | | |
| | | AGGCGCCGCCGCACCCCCTCTA | | | |
| | | GCGGGCGCGGGGCGAAGCGGT | | | |
| | | GCGGCGCCGGCAGGAAGGAAA | | | |
| | | TGGGCGGGGAGGGCCTTCGTGC | | | |
| | | GTCGCCGCGCCGCCGTCCCCTT | | | |
| | | CTCCCTCTCCAGCCTCGGGGCT | | | |
| | | GTCCGCGGGGGGACGGCTGCCT | | | |
| | | TCGGGGGGGACGGGGCAGGGC | | | |
| | | GGGGTTCGGCTTCTGGCGTGTG | | | |
| | | ACCGGCGGCTCTAGAGCCTCTG | | | |
| | | CTAACCATGTTCATGCCTTCTTC | | | |
| | | TTTTTCCTACAGCTCCTGGGCA | | | |
| | | ACGTGCTGGTTATTGTGCTGTCT | | | |
| | | CATCATTTTGGCAAAGAATTCC | | | |
| | | TCGAGCGGCCGCCAGTGTGATG | | | |
| | | GATATCGGATCAGGCTTGAACT | | | |
| | | TCCCACACGTGTTATTTGGCCC | | | |
| | | ACATTGTGTTTGAAGAAACTTT | | | |
| | | GGGATTGGTTGCCAGTGCTTAA | | | |
| | | AAGTTAGGACTTAGAAAATGGA | | | |
| | | TTTCCTGGCAGGACGCGGTGGC | | | |
| | | TCATGCCCATAATCTCAGCACT | | | |
| | | TTGGGAGGCCTAGGAAGGTGG | | | |
| | | ATCACCTGAGGTCCGGAGTTCA | | | |
| | | AGACTAACCTGGCCAACATGGT | | | |
| | | GAAACCCAGTATCTACTAAAAA | | | |
| | | ATACAAAAAAAAAAAAAAAAG | | | |
| | | AAGAAGAAGAAGAAGAAGAAG | | | |
| | | AAGAAGAAGAAGAAGAAGAAG | | | |
| | | AAGAAGAAGAAGAAGAAGAAG | | | |
| | | AAGAAGAAGAAGAAGAAGAAG | | | |
| | | AAGAAAATAAAGAAAAGTTAG | | | |
| | | CCGGGCGTGGTGTCGCGCGCCT | | | |
| | | GTAATCCCAGCTACTCCAGAGG | | | |
| | | CTGCGGCAGGAGAATCGCTTGA | | | |
| | | GCCCGGGAGGCAGAGGTTGCAT | | | |
| | | TAAGCCAAGATCGCCCAATGCA | | | |
| | | CTCCGGCCTGGGCGACAGAGCA | | | |
| | | AGACTCCGTCTCAAAAAATAAT | | | |
| | | AATAATAAATAAAAATAAAAA | | | |
| | | ATAAAATGGATTTCCCAGCATC | | | |
| | | TCTGGAAAAATAGGCAAGTGTG | | | |
| | | GCCATGATGGTCCTGATCCGCT | | | |
| | | AGCGCTACTAACTTCAGCCTGC | | | |
| | | TGAAGCAGGCTGGAGACGTGG | | | |
| | | AGGAGAACCCTGGACCTGGACC | | | |
| | | GGTCGCCACCATGGTGAGCAAG | | | |
| | | GGCGAGGAGCTGTTCACCGGGG | | | |
| | | TGGTGCCCATCCTGGTCGAGCT | | | |
| | | GGACGGCGACGTAAACGGCCA | | | |
| | | CAAGTTCAGCGTGTCCGGCGAG | | | |
| | | GGCGAGGGCGATGCCACCTACG | | | |
| | | GCAAGCTGACCCTGAAGTTCAT | | | |
| | | CTGCACCACCGGCAAGCTGCCC | | | |
| | | GTGCCCTGGCCCACCCTCGTGA | | | |
| | | CCACCCTGACCTACGGCGTGCA | | | |
| | | GTGCTTCAGCCGCTACCCCGAC | | | |
| | | CACATGAAGCAGCACGACTTCT | | | |
| | | TCAAGTCCGCCATGCCCGAAGG | | | |
| | | CTACGTCCAGGAGCGCACCATC | | | |
| | | TTCTTCAAGGACGACGGCAACT | | | |
| | | ACAAGACCCGCGCCGAGGTGA | | | |
| | | AGTTCGAGGGCGACACCCTGGT | | | |
| | | GAACCGCATCGAGCTGAAGGG | | | |
| | | CATCGACTTCAAGGAGGACGGC | | | |
| | | AACATCCTGGGGCACAAGCTGG | | | |
| | | AGTACAACTACAACAGCCACAA | | | |
| | | CGTCTATATCATGGCCGACAAG | | | |
| | | CAGAAGAACGGCATCAAGGTG | | | |
| | | AACTTCAAGATCCGCCACAACA | | | |
| | | TCGAGGACGGCAGCGTGCAGCT | | | |
| | | CGCCGACCACTACCAGCAGAAC | | | |
| | | ACCCCCATCGGCGACGGCCCCG | | | |
| | | TGCTGCTGCCCGACAACCACTA | | | |
| | | CCTGAGCACCCAGTCCGCCCTG | | | |
| | | AGCAAAGACCCCAACGAGAAG | | | |
| | | CGCGATCACATGGTCCTGCTGG | | | |
| | | AGTTCGTGACCGCCGCCGGGAT | | | |
| | | CACTCTCGGCATGGACGAGCTG | | | |
| | | TACAAGTAAAGCGGCCGCCACC | | | |
| | | GCGGTGGAGCTCGAATTAATTC | | | |
| | | ATCGATGATGATCCAGACATGA | | | |
| | | TAAGATACATTGATGAGTTTGG | | | |
| | | ACAAACCACAACTAGAATGCA | | | |
| | | GTGAAAAAAATGCTTTATTTGT | | | |
| | | GAAATTTGTGATGCTATTGCTTT | | | |
| | | ATTTGTAACCATTATAAGCTGC | | | |
| | | AATAAACAAGTTAACAACAAC | | | |
| | | AATTGCATTCATTTTATGTTTCA | | | |
| | | GGTTCAGGGGGAGGTGTGGGA | | | |
| | | GGTTTTTTAAAGCAAGTAAAAC | | | |
| | | CTCTACAAATGTGGTATGGCTG | | | |
| | | ATTATGATCCTCTAGAGTCGGT | | | |
| | | GGGCCTCGGGGGCGGGTGCGG | | | |
| | | GGTCGGCGGGGCCGCCCCGGGT | | | |
| | | GGCTTCGGTCGGAGCCATGGGG | | | |
| | | TCGTGCGCTCCTTTCGGTCGGG | | | |
| | | CGCTGCGGGTCGTGGGGGGGGC | | | |
| | | GTCAGGCACCGGGCTTGCGGGT | | | |
| | | CATGCACCAGGTGCGCGGTCCT | | | |
| | | TCGGGCACCTCGACGTCGGCGG | | | |
| | | TGACGGTGAAGCCGAGCCGCTC | | | |
| | | GTAGAAGGGGAGGTTGCGGGG | | | |
| | | CGCGGAGGTCTCCAGGAAGGC | | | |
| | | GGGCACCCCGGCGCGCTCGGCC | | | |
| | | GCCTCCACTCCGGGGAGCACGA | | | |
| | | CGGCGCTGCCCAGACCCTTGCC | | | |
| | | CTGGTGGTCGGGCGAGACGCCG | | | |
| | | ACGGTGGCCAGGAACCACGCG | | | |
| | | GGCTCCTTGGGCCGGTGCGGCG | | | |
| | | CCAGGAGGCCTTCCATCTGTTG | | | |
| | | CTGCGCGGCCAGCCGGGAACCG | | | |
| | | CTCAACTCGGCCATGCGCGGGC | | | |
| | | CGATCTCGGCGAACACCGCCCC | | | |
| | | CGCTTCGACGCTCTCCGGCGTG | | | |
| | | GTCCAGACCGCCACCGCGGCGC | | | |
| | | CGTCGTCCGCGACCCACACCTT | | | |
| | | GCCGATGTCGAGCCCGACGCGC | | | |
| | | GTGAGGAAGAGTTCTTGCAGCT | | | |
| | | CGGTGACCCGCTCGATGTGGCG | | | |
| | | GTCCGGGTCGACGGTGTGGCGC | | | |
| | | GTGGCGGGGTAGTCGGCGAAC | | | |
| | | GCGGCGGCGAGGGTGCGTACG | | | |
| | | GCCCGGGGGACGTCGTCGCGGG | | | |
| | | TGGCGAGGCGCACCGTGGGCTT | | | |
| | | GTACTCGGTCATGGAAGGTCGT | | | |
| | | CTCCTTGTGAGGGGTCAGGGGC | | | |
| | | GTGGGTCAGGGGATGGTGGCG | | | |
| | | GCACCGGTCGTGGCGGCCGACC | | | |
| | | TGCAGGCATGCAAGCTTTTTGC | | | |
| | | AAAAGCCTAGGCCTCCAAAAA | | | |
| | | AGCCTCCTCACTACTTCTGGAA | | | |
| | | TAGCTCAGAGGCCGAGGCGGCC | | | |
| | | TCGGCCTCTGCATAAATAAAAA | | | |
| | | AAATTAGTCAGCCATGGGGCGG | | | |
| | | AGAATGGGCGGAACTGGGCGG | | | |
| | | AGTTAGGGGGGGGATGGGCGG | | | |
| | | AGTTAGGGGGGGGACTATGGTT | | | |
| | | GCTGACTAATTGAGATGCATGC | | | |
| | | TTTGCATACTTCTGCCTGCTGGG | | | |
| | | GAGCCTGGGGACTTTCCACACC | | | |
| | | TGGTTGCTGACTAATTGAGATG | | | |
| | | CATGCTTTGCATACTTCTGCCTG | | | |
| | | CTGGGGAGCCTGGGGACTTTCC | | | |
| | | ACACCCTAACTGACACACATTC | | | |
| | | CACAGAATTCAAGTGATCTCCA | | | |
| | | AAAAATAAGTACTTTTTGACTG | | | |
| | | TAAATAAAATTGTAAGGAGTAA | | | |
| | | AAAGTACTTTTTTTTCTAAAAA | | | |
| | | AATGTAATTAAGTAAAAGTAAA | | | |
| | | AGTATTGATTTTTAATTGTACTC | | | |
| | | AAGTAAAGTAAAAATCCCCAA | | | |
| | | AAATAATACTTAAGTACAGTAA | | | |
| | | TCAAGTAAAATTACTCAAGTAC | | | |
| | | TTTACACCTCTG | | | |

### REFERENCES

1. Roos, R. A. C. Huntington's disease: a clinical review. Orphanet journal of rare diseases 5, (2010).
2. Bates, G. P., Dorsey, R., Gusella, J. F., Hayden, M. R., et al. Huntington disease. Nature Reviews Disease Primers vol. 1 (2015).
3. Frank, S. Treatment of Huntington's Disease. Neurotherapeutics 11, 153 (2014).
4. Langbehn, D. R., Hayden, M. R., Paulsen, J. S., Johnson, H., et al. CAG-repeat length and the age of onset in Huntington disease (HD): a review and validation study of statistical approaches. American journal of medical genetics. Part B, Neuropsychiatric genetics: the official publication of the International Society of Psychiatric Genetics 153B, 397-408 (2010).
5. Gusella, J. F. CAG Repeat Not Polyglutamine Length Determines Timing of Huntington's Disease Onset. (2019) doi:10.1016/j.cell.2019.06.036.
6. Gatto, E. M., Rojas, N. G., Persi, G., Etcheverry, J. L., et al. Huntington disease: Advances in the understanding of its mechanisms. Clinical parkinsonism & related disorders 3, (2020).
7. Swami, M., Hendricks, A. E., Gillis, T., Massood, T., et al. Somatic expansion of the Huntington's disease CAG repeat in the brain is associated with an earlier age of disease onset. Human Molecular Genetics 18, 3039-3047 (2009).
8. Kacher, R., Lejeune, F. X., Noël, S., Cazeneuve, C., et al. Propensity for somatic expansion increases over the course of life in Huntington disease. eLife 10, (2021).
9. Pinto, R. M., Arning, L., Giordano, J. v., Razghandi, P., et al. Patterns of CAG repeat instability in the central nervous system and periphery in Huntington's disease and in spinocerebellar ataxia type 1. Human molecular genetics 29, 2551-2567 (2020).
10. Oura, S., Noda, T., Morimura, N., Hitoshi, S., et al. Precise CAG repeat contraction in a Huntington's Disease mouse model is enabled by gene editing with SpCas9-NG. Communications Biology 4, (2021).
11. Cinesi, C., Aeschbach, L., Yang, B. & Dion, V. Contracting CAG/CTG repeats using the CRISPR-Cas9 nickase. Nature Communications 7, 1-10 (2016).
12. Anzalone, A. v, Randolph, P. B., Davis, J. R., Sousa, A. A., et al. Search-and-replace genome editing without double-strand breaks or donor DNA. Nature 576, 149 (2019).
13. Komor, A. C., Zhao, K. T., Packer, M. S., Gaudelli, N. M., et al. Improved base excision repair inhibition and bacteriophage Mu Gam protein yields C:G-to-T:A base editors with higher efficiency and product purity. Science Advances 3, eaao4774-eaao4774 (2017).
14. Wright, G. E. B., Collins, J. A., Kay, C., McDonald, C., et al. Length of uninterrupted CAG repeats, independent of polyglutamine size, results in increased somatic instability and hastened age of onset in Huntington disease. Am. J. Hum. Genet. 104 (6), 1116-1126 (2019).
15. Costa, M. D. C., Magalhães, P., Guimarães, L., Maciel, P., et al. The CAG repeat at the Huntington disease gene in the Portuguese population: insights into its dynamics and to the origin of the mutation. Journal of Human Genetics 2005 51:3 51, 189-195 (2006).
16. Monteys, A. M., Ebanks, S. A., Keiser, M. S. & Davidson, B. L. CRISPR/Cas9 Editing of the Mutant Huntingtin Allele In Vitro and In Vivo. Molecular Therapy 25, 12-23 (2017).
17. Dabrowska, M., Juzwa, W., Krzyzosiak, W. J. & Olejniczak, M. Precise Excision of the CAG Tract from the Huntingtin Gene by Cas9 Nickases. Frontiers in Neuroscience 12, (2018).
18. Yang, S., Li, S. & Li, X.-J. CRISPR/Cas9-mediated gene editing ameliorates neurotoxicity in mouse model of Huntington's disease. J Clin Invest 127, 2719 (2017).
19. Ekman, F. K., Ojala, D. S., Adil, M. M., Lopez, P. A., et al. CRISPR-Cas9-Mediated Genome Editing Increases Lifespan and Improves Motor Deficits in a Huntington's Disease Mouse Model. Molecular Therapy—Nucleic Acids 17, 829-839 (2019).
20. Shin, J. W., Kim, K.-H., Chao, M. J., Atwal, R. S., et al. Permanent inactivation of Huntington's disease mutation by personalized allele-specific CRISPR/Cas9. doi:10.1093/hmg/ddw286.
21. Saudou, F. & Humbert, S. The Biology of Huntingtin. Neuron vol. 89 910-926 (2016).
22. A Study to Evaluate the Safety, Tolerability, Pharmacokinetics, and Pharmacodynamics of RO7234292 (ISIS 443139) in Huntington's Disease Patients Who Participated in Prior Investigational Studies of RO7234292 (ISIS 443139). ClinicalTrials.gov. clinicaltrials.gov/ct2/show/NCT03342053.
23. Kingwell, K. Double setback for ASO trials in Huntington disease. Nature Reviews. Drug Discovery 20, 412-413 (2021).
24. Komor, A. C., Kim, Y. B., Packer, M. S., Zuris, J. A., et al. Programmable editing of a target base in genomic DNA without double-stranded DNA cleavage. Nature 533, 420-424 (2016).
25. Gaudelli, N. M., Komor, A. C., Rees, H. A., Packer, M. S., et al. Programmable base editing of A·T to G·C in genomic DNA without DNA cleavage. Nature 551, 464-471 (2017).
26. Nelson, J. W., Randolph, P. B., Shen, S. P., Everette, K. A., et al. Engineered pegRNAs improve prime editing efficiency. Nature biotechnology 40, 402-410 (2022).
27. Chen, P. J., Hussmann, J. A., Yan, J., Knipping, F., et al. Enhanced prime editing systems by manipulating cellular determinants of editing outcomes. Cell 184, 5635-5652.e29 (2021).
28. Choi, J., Chen, W., Suiter, C. C., Lee, C., et al. Precise genomic deletions using paired prime editing. doi:10.1101/2020.12.30.424891.
29. Anzalone, A. V., Gao, X. D., Podracky, C. J., Nelson, A. T., et al. Programmable deletion, replacement, integration and inversion of large DNA sequences with twin prime editing. Nature biotechnology 40, (2022).
30. Chen, F., Ding, X., Feng, Y., Seebeck, T., et al. Targeted activation of diverse CRISPR-Cas systems for mammalian genome editing via proximal CRISPR targeting. Nature Communications 8, 14958 (2017).
31. Narain, Y., Wyttenbach, A., Rankin, J., Furlong, R. A., et al. Original articles A molecular investigation of true dominance in Huntington's disease. doi:10.1136/jmg.36.10.739.
32. Kawakami, K. Tol2: A versatile gene transfer vector in vertebrates. Genome Biology vol. 8 S7 (2007).
33. Arbab, M., Srinivasan, S., Hashimoto, T., Geijsen, N., et al. Cloning-free CRISPR. Stem Cell Reports 5, 908-917 (2015).
34. Massey, T. H. & Jones, L. The central role of DNA damage and repair in CAG repeat diseases. DMM Disease Models and Mechanisms 11, dmm031930-dmm031930 (2018).
35. Liu, Y. & Wilson, S. H. DNA base excision repair: A mechanism of trinucleotide repeat expansion. Trends in Biochemical Sciences (2012) doi:10.1016/j.tibs.2011.12.002.
36. Wright, G. E. B., Black, H. F., Collins, J. A., Gall-Duncan, T., et al. Interrupting sequence variants and age of onset in Huntington's disease: clinical implications and emerging therapies. The Lancet Neurology 19, 930-939 (2020).
37. Findlay Black, H., Wright, G. E. B., Collins, J. A., Caron, N., et al. Frequency of the loss of CAA interruption in the HTT CAG tract and implications for Huntington disease in the reduced penetrance range. Genetics in Medicine 22, 2108-2113 (2020).
38. Rolfsmeier, M. L. & Lahue, R. S. Stabilizing Effects of Interruptions on Trinucleotide Repeat Expansions in Saccharomyces cerevisiae. Molecular and Cellular Biology (2000) doi:10.1128/mcb.20.1.173-180.2000.
39. McGurk Leeanne, Rifai Olivia M, Sherbakova Oksana, Perlegos Alexandra E., Byrns chiuna N., Carranza, Zhou Henry W., Kim Hyung-Jun, Zhu Yongqing, B. N. M. Toxicity of pathogenic ataxin-2 in Drosophila shows dependence on a pure CAG repeat sequence. Human Molecular Genetics 1-5 (2021).
40. Koblan, L. W., Doman, J. L., Wilson, C., Levy, J. M., et al. Improving cytidine and adenine base editors by expression optimization and ancestral reconstruction. Nat Biotechnol 36, 843-846 (2018).
41. Gehrke, J. M., Cervantes, O., Clement, M. K., Wu, Y., et al. An APOBeC3A-Cas9 base editor with minimized bystander and off-target activities. Nature Biotechnology 36, (2018).
42. Huang, T. P., Zhao, K. T., Miller, S. M., Gaudelli, N. M., et al. Circularly permuted and PAM-modified Cas9 variants broaden the targeting scope of base editors. Nature Biotechnology 1 (2019) doi:10.1038/s41587-019-0134-y.
43. Komor, A. C., Zhao, K. T., Packer, M. S., Gaudelli, N. M., et al. Improved base excision repair inhibition and bacteriophage Mu Gam protein yields C:G-to-T:A base editors with higher efficiency and product purity. Science Advances 3, eaao4774 (2017).
44. Thuronyi, B. W., Koblan, L. W., Levy, J. M., Yeh, W.-H., et al. Continuous evolution of base editors with expanded target compatibility and improved activity. Nature Biotechnology 1 (2019) doi:10.1038/s41587-019-0193-0.
45. Arbab, M., Shen, M. W., Mok, B., Wilson, C., et al. Determinants of Base Editing Outcomes from Target Library Analysis and Machine Learning. Cell 182, 463-480.e30 (2020).
46. Miller, S., Wang, T., Randolph, P., Arbab, M., et al. Continuous evolution of SpCas9 variants compatible with non-G PAMs. Nature Biotechnology 38, 471-481 (2020).
47. Nishimasu, H., Shi, X., Ishiguro, S., Gao, L., et al. Engineered CRISPR-Cas9 nuclease with expanded targeting space. Science 361, 1259-1262 (2018).
48. Iyer, R. R., Pluciennik, A., Napierala, M. & Wells, R. D. DNA triplet repeat expansion and mismatch repair. Annual Review of Biochemistry vol. 84 199-226 (2015).
49. Budworth, H. & McMurray, C. T. A Brief History of Triplet Repeat Diseases. Methods Mol. Biol. (2013). doi:10.1007/978-1-62703-411-1_1.
50. Bidichandani, S. I. & Delatycki, M. B. Friedreich Ataxia. GeneReviews®(2017).
51. Cnop, M., Mulder, H. & Igoillo-Esteve, M. Diabetes in Friedreich ataxia. Journal of neurochemistry 126 Suppl, 94-102 (2013).
52. Pandolfo, M. Friedreich Ataxia: New Pathways. doi:10.1177/0883073812448534.
53. Reetz, K., Dogan, I., Costa, A. S., Dafotakis, M., et al. Biological and clinical characteristics of the European Friedreich's Ataxia Consortium for Translational Studies (EFACTS) cohort: a cross-sectional analysis of baseline data. Articles Lancet Neurol 14, 174-182 (2015).
54. Cook, A. & Giunti, P. Friedreich's ataxia: clinical features, pathogenesis and management. British Medical Bulletin 124, 19-30 (2017).
55. Silva, A. M., Brown, J. M., Buckle, V. J., Wade-Martins, R., et al. Expanded GAA repeats impair FXN gene expression and reposition the FXN locus to the nuclear lamina in single cells. Human Molecular Genetics 24, 3457 (2015).
56. Lazaropoulos, M., Dong, Y., Clark, E., Greeley, N. R., et al. Frataxin levels in peripheral tissue in Friedreich ataxia. Annals of Clinical and Translational Neurology 2, 831-842 (2015).
57. Delatycki, M. B. & Bidichandani, S. I. Friedreich ataxia-pathogenesis and implications for therapies. Neurobiology of disease 132, (2019).
58. Long, A., Napierala, J. S., Polak, U., Hauser, L., et al. Somatic instability of the expanded GAA repeats in Friedreich's ataxia. PloS one 12, (2017).
59. Neil, A. J., Hisey, J. A., Quasem, I., McGinty, R. J., et al. Replication-independent instability of Friedreich's ataxia GAA repeats during chronological aging. Proceedings of the National Academy of Sciences of the United States of America 118, (2021).
60. De Biase, I., Rasmussen, A., Endres, D., Al-Mahdawi, S., et al. Progressive GAA expansions in dorsal root ganglia of Friedreich's ataxia patients. Annals of neurology 61, 55-60 (2007).
61. Pinto, R. M., Arning, L., Giordano, J. V., Razghandi, P., et al. Patterns of CAG repeat instability in the central nervous system and periphery in Huntington's disease and in spinocerebellar ataxia type 1. Human molecular genetics 29, 2551-2567 (2020).
62. Kacher, R., Lejeune, F. X., Noël, S., Cazeneuve, C., et al. Propensity for somatic expansion increases over the course of life in huntington disease. eLife 10, (2021).
63. Swami, M., Hendricks, A. E., Gillis, T., Massood, T., et al. Somatic expansion of the Huntington's disease CAG repeat in the brain is associated with an earlier age of disease onset. Human molecular genetics 18, 3039-3047 (2009).
64. Rocca, C. J., Rainaldi, J. N., Sharma, J., Shi, Y., et al. CRISPR-Cas9 Gene Editing of Hematopoietic Stem Cells from Patients with Friedreich's Ataxia. Molecular Therapy—Methods & Clinical Development 17, 1026-1036 (2020).
65. Mazzara, P. G., Muggeo, S., Luoni, M., Massimino, L., et al. Frataxin gene editing rescues Friedreich's ataxia pathology in dorsal root ganglia organoid-derived sensory neurons. Nature Communications 11, 1-18 (2020).
66. Li, Y., Polak, U., Bhalla, A. D., Rozwadowska, N., et al. Excision of Expanded GAA Repeats Alleviates the Molecular Phenotype of Friedreich's Ataxia. Molecular Therapy 23, 1055-1065 (2015).
67. Mc Vey, M. & Lee, S. E. MMEJ repair of double-strand breaks (director's cut): deleted sequences and alternative endings. Trends in genetics: TIG 24, 529-538 (2008).
68. Jeggo, P. A. 5 DNA Breakage and Repair. Advances in Genetics 38, 185-218 (1998).
69. Fishman-Lobell, J., Rudin, N. & Haber, J. E. Two alternative pathways of double-strand break repair that are kinetically separable and independently modulated. Molecular and cellular biology 12, 1292-1303 (1992).
70. Rudin, N. & Haber, J. E. Efficient repair of HO-induced chromosomal breaks in Saccharomyces cerevisiae by recombination between flanking homologous sequences. Molecular and cellular biology 8, 3918-3928 (1988).
71. Enache, O. M., Rendo, V., Abdusamad, M., Lam, D., et al. Cas9 activates the p53 pathway and selects for p53-inactivating mutations. Nature genetics 52, 662-668 (2020).
72. Neil, A. J., Hisey, J. A., Quasem, I., McGinty, R. J., et al. Replication-independent instability of Friedreich's ataxia GAA repeats during chronological aging. Proceedings of the National Academy of Sciences of the United States of America 118, (2021).
73. Anzalone, A. V., Randolph, P. B., Davis, J. R., Sousa, A. A., et al. Search-and-replace genome editing without double-strand breaks or donor DNA. Nature (2019) doi:10.1038/s41586-019-1711-4.
74. Gaudelli, N. M., Komor, A. C., Rees, H. A., Packer, M. S., et al. Programmable base editing of T to G C in genomic DNA without DNA cleavage. Nature 551, 464-471 (2017).
75. Richter, M. F., Zhao, K. T., Eton, E., Lapinaite, A., et al. Phage-assisted evolution of an adenine base editor with improved Cas domain compatibility and activity. Nature Biotechnology (2020) doi:10.1038/s41587-020-0453-z.
76. Arbab, M., Shen, M. W., Mok, B., Wilson, C., et al. Determinants of Base Editing Outcomes from Target Library Analysis and Machine Learning. Cell 182, 463-480.e30 (2020).
77. Newby, G. A., Yen, J. S., Woodard, K. J., Mayuranathan, T., et al. Base editing of haematopoietic stem cells rescues sickle cell disease in mice. Nature 2021 595:7866 595, 295-302 (2021).
78. Koblan, L. W., Erdos, M. R., Wilson, C., Cabral, W. A., et al. In vivo base editing rescues Hutchinson-Gilford progeria syndrome in mice. Nature 1-7 (2021) doi:10.1038/s41586-020-03086-7.
79. Nethisinghe, S., Kesavan, M., Ging, H., Labrum, R., et al. Interruptions of the FXN GAA Repeat Tract Delay the Age at Onset of Friedreich's Ataxia in a Location Dependent Manner. International Journal of Molecular Sciences 2021, Vol. 22, Page 7507 22, 7507 (2021).
80. Al-Mahdawi, S., Ging, H., Bayot, A., Cavalcanti, F., et al. Large interruptions of GAA repeat expansion mutations in Friedreich ataxia are very rare. Frontiers in Cellular Neuroscience 12, (2018).
81. Khristich, A. N., Armenia, J. F., Matera, R. M., Kolchinski, A. A., et al. Large-scale contractions of Friedreich's ataxia GAA repeats in yeast occur during DNA replication due to their triplex-forming ability. Proceedings of the National Academy of Sciences of the United States of America 117, 1628-1637 (2020).
82. Soragni, E., Herman, D., Dent, S. Y. R., Gottesfeld, J. M., et al. Long intronic GAATTC repeats induce epigenetic changes and reporter gene silencing in a molecular model of Friedreich ataxia. Nucleic Acids Research 36, 6056-6065 (2008).
83. Greene, E., Mahishi, L., Entezam, A., Kumari, D., et al. Repeat-induced epigenetic changes in intron 1 of the frataxin gene and its consequences in Friedreich ataxia. Nucleic Acids Research 35, 3383-3390 (2007).
84. Li, J., Rozwadowska, N., Clark, A., Fil, D., et al. Excision of the expanded GAA repeats corrects cardiomyopathy phenotypes of iPSC-derived Friedreich's ataxia cardiomyocytes. Stem cell research 40, 101529 (2019).
85. Li, Y., Li, J., Wang, J., Zhang, S., et al. Premature transcription termination at the expanded GAA repeats and aberrant alternative polyadenylation contributes to the Frataxin transcriptional deficit in Friedreich's ataxia. Human molecular genetics (2022) doi:10.1093/HMG/DDAC134.
86. Jiralerspong, S., Liu, Y., Montermini, L., Stifani, S., et al. Frataxin shows developmentally regulated tissue-specific expression in the mouse embryo. Neurobiology of disease 4, 103-113 (1997).
87. Puspasari, N., Rowley, S. M., Gordon, L., Lockhart, P. J., et al. Long range regulation of human FXN gene expression. PloS one 6, (2011).
88. Komor, A. C., Kim, Y. B., Packer, M. S., Zuris, J. A., et al. Programmable editing of a target base in genomic DNA without double-stranded DNA cleavage. Nature 533, 420-424 (2016).
89. Nelson, J. W., Randolph, P. B., Shen, S. P., Everette, K. A., et al. Engineered pegRNAs improve prime editing efficiency. Nature biotechnology 40, 402-410 (2022).
90. Chen, P. J., Hussmann, J. A., Yan, J., Knipping, F., et al. Enhanced prime editing systems by manipulating cellular determinants of editing outcomes. Cell 184, 5635-5652.e29 (2021).
91. Choi, J., Chen, W., Suiter, C. C., Lee, C., et al. Precise genomic deletions using paired prime editing. Nature Biotechnology 2021 40:2 40, 218-226 (2021).
92. Anzalone, A. V., Gao, X. D., Podracky, C. J., Nelson, A. T., et al. Programmable deletion, replacement, integration and inversion of large DNA sequences with twin prime editing. Nature biotechnology 40, (2022).
93. Tao, R., Wang, Y., Jiao, Y., Hu, Y., et al. Bi-PE: bi-directional priming improves CRISPR/Cas9 prime editing in mammalian cells. Nucleic Acids Research 50, 6423-6434 (2022).
94. Chen, F., Ding, X., Feng, Y., Seebeck, T., et al. Targeted activation of diverse CRISPR-Cas systems for mammalian genome editing via proximal CRISPR targeting. Nature communications 8, 14958 (2017).
95. Shishkin, A. A., Voineagu, I., Matera, R., Cherng, N., et al. Large-scale expansions of Friedreich's ataxia GAA repeats in yeast. Molecular cell 35, 82-92 (2009).
96. Ohshima, K., Sakamoto, N., Labuda, M., Poirier, J., et al. A nonpathogenic GAAGGA repeat in the Friedreich gene: Implications for pathogenesis. Neurology 53, 1854 (1999).
97. Sakamoto, N., Larson, J. E., Iyer, R. R., Montermini, L., et al. GGATCC-interrupted Triplets in Long GAATTC Repeats Inhibit the Formation of Triplex and Sticky DNA Structures, Alleviate Transcription Inhibition, and Reduce Genetic Instabilities* Downloaded from. Journal of Biological Chemistry 276, 27178-27187 (2001).
98. Chandran, V., Gao, K., Swarup, V., Versano, R., et al. Inducible and reversible phenotypes in a novel mouse model of Friedreich's ataxia. eLife 6, (2017).
99. Larimar Therapeutics Provides Update on CTI-1601 Clinical Program-Larimar Therapeutics, Inc. investors.larimartx.com/news-releases/news-release-details/larimar-therapeutics-provides-update-cti-1601-clinical-program.
100. Belbellaa, B., Reutenauer, L., Messaddeq, N., Monassier, L., et al. High Levels of Frataxin Overexpression Lead to Mitochondrial and Cardiac Toxicity in Mouse Models. Molecular Therapy—Methods and Clinical Development 19, 120-138 (2020).
101. Huichalaf, C., Perfitt, T. L., Kuperman, A., Gooch, R., et al. In vivo overexpression of frataxin causes toxicity mediated by iron-sulfur cluster deficiency. Molecular Therapy—Methods and Clinical Development 24, 367-378 (2022).
102. Miller, S. M., Wang, T., Randolph, P. B., Arbab, M., et al. Continuous evolution of SpCas9 variants compatible with non-G PAMs. doi:10.1038/s41587-020-0412-8.
103. Walton, R. T., Christie, K. A., Whittaker, M. N. & Kleinstiver, B. P. Unconstrained genome targeting with near-PAMless engineered CRISPR-Cas9 variants. Science 368, 290-296 (2020).
104. Nishimasu, H., Shi, X., Ishiguro, S., Gao, L., et al. Engineered CRISPR-Cas9 nuclease with expanded targeting space. Science 361, 1259-1262 (2018).
105. Chatterjee, P., Lee, J., Nip, L., Koseki, S. R. T., et al. A Cas9 with PAM recognition for adenine dinucleotides. Nature Communications 11, 1-6 (2020).
106. Massey, T. H. & Jones, L. The central role of DNA damage and repair in CAG repeat diseases. Disease models & mechanisms 11, dmm031930 (2018).
107. Liu, Y. & Wilson, S. H. DNA base excision repair: a mechanism of trinucleotide repeat expansion. Trends in Biochemical Sciences 37, 162-172 (2012).
108. Oura, S., Noda, T., Morimura, N., Hitoshi, S., et al. Precise CAG repeat contraction in a Huntington's Disease mouse model is enabled by gene editing with SpCas9-NG. Communications Biology 4, (2021).
109. Kawakami, K. Tol2: A versatile gene transfer vector in vertebrates. Genome Biology vol. 8 S7 (2007).
110. Arbab, M., Srinivasan, S., Hashimoto, T., Geijsen, N., et al. Cloning-free CRISPR. Stem Cell Reports 5, 908-917 (2015).

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

What is claimed is:

1. A pegRNA comprising a spacer sequence comprising the nucleic acid sequence:
(SEQ ID NO: 381)
GACCCTGGAAAAGCTGATGA;
(SEQ ID NO: 382)
GCTGCTGCTGGAAGGACTTG;
(SEQ ID NO: 383)
GCTGCTGCTGCTGCTGCTGGA;
(SEQ ID NO: 384)
GCTGCTGCTGCTGCTGCTGC;
(SEQ ID NO: 385)
GGCGGCGGCGGCGGCGGTGG;
(SEQ ID NO: 386)
TGAGGAAGCTGAGGAGGCGG;
or
(SEQ ID NO: 387)
GGCGGCTGAGGAAGCTGAGG,
or a nucleic acid sequence at least 80%, at least 85%, at least 90%, at least 95%, at least 96%, at least 97%, at least 98%, or at least 99% identical to any one of SEQ ID NOs: 381-387.

2. The pegRNA of claim 1, wherein the pegRNA comprises the spacer sequence GACCCTGGAAAAGCTGAT (SEQ ID NO: 873), or a spacer sequence at least 80%, at least 85%, at least 90%, at least 95%, at least 96%, at least 97%, at least 98%, or at least 99% identical to SEQ ID NO: 873.

3. The pegRNA of claim 1 or 2, wherein the pegRNA comprises a primer binding site (PBS) of about 8 to about 16 nucleotides in length.

4. The pegRNA of any one of claims 1-3, wherein the pegRNA comprises a PBS of about 8, about 9, about 10, about 11, about 12, about 13, about 14, about 15, or about 16 nucleotides in length.

5. The pegRNA of any one of claims 1-4, wherein the pegRNA comprises a PBS of about 10 nucleotides in length.

6. The pegRNA of any one of claims 1-5, wherein the pegRNA comprises a reverse transcription template comprising a sequence of about 15 to about 35 nucleotides, wherein the sequence encodes a sequence that comprises one or more nucleotide edits compared to a sequence directly upstream of a CAG repeat in exon 1 of the HTT gene.

7. The pegRNA of claim 6, wherein the one or more nucleotide edits comprises a PAM mutation that alters a PAM sequence.

8. The pegRNA of claim 7, wherein the PAM sequence is NGG, wherein N is any one of nucleotides A, G, C, or T.

9. The pegRNA of any one of claims 1-8, wherein the pegRNA comprises a reverse transcription template comprising a sequence of about 26 nucleotides, wherein the sequence encodes a sequence that comprises one or more nucleotide edits compared to a sequence directly upstream of a CAG repeat in exon 1 of the HTT gene.

10. The pegRNA of any one of claims 1-9, wherein the pegRNA comprises a reverse transcription template comprising an edit template encoding or comprising one or more repeats of the trinucleotide sequence CAG.

11. The pegRNA of claim 10, wherein the edit template encodes or comprises 4-35 repeats of the trinucleotide sequence CAG, or wherein the edit template encodes or comprises 4-10 repeats of the trinucleotide sequence CAG.

12. The pegRNA of claim 11, wherein the pegRNA comprises a reverse transcription template comprising an edit template encoding or comprising the nucleotide sequence CAGCAGCAGCAG (SEQ ID NO: 874), or a nucleotide sequence at least 80%, at least 85%, at least 90%, at least 95%, at least 96%, at least 97%, at least 98%, or at least 99% identical to SEQ ID NO: 874.

13. The pegRNA of claim 11, wherein the pegRNA comprises a reverse transcription template comprising an edit template encoding or comprising the nucleotide sequence CAGCAGCAGCAGCAGCAGCAGCAGCAG (SEQ ID NO: 875), or a nucleotide sequence at least 80%, at least 85%, at least 90%, at least 95%, at least 96%, at least 97%, at least 98%, or at least 99% identical to SEQ ID NO: 875.

14. The pegRNA of any one of claims 10-13, wherein the edit template further encodes or comprises one or more nucleotides of the trinucleotide sequence CAA.

15. The pegRNA of any one of claims 10-14, wherein the pegRNA comprises a reverse transcription template comprising an edit template encoding or comprising the nucleotide sequence CAGCAGCAGCAGCAACAACAA (SEQ ID NO: 876), or a nucleic acid sequence at least 80%, at least 85%, at least 90%, at least 95%, at least 96%, at least 97%, at least 98%, or at least 99% identical to SEQ ID NO: 876.

16. The pegRNA of any one of claims 1-15, wherein the pegRNA comprises a reverse transcription template comprising a homology arm of about 16 to about 50 nucleotides in length, wherein the homology arm is complementary to a sequence directly downstream of a CAG repeat in exon 1 of a wildtype HTT gene in the coding strand.

17. The pegRNA of claim 16, wherein the homology arm is about 31 nucleotides in length.

18. The pegRNA of claim 16, wherein the homology arm is about 40 nucleotides in length.

19. The pegRNA of any one of claims 1-18, wherein the pegRNA is an engineered pegRNA (epegRNA).

20. The pegRNA of any one of claims 1-19, wherein the pegRNA comprises at its 3′ end a structural motif that improves stability of the pegRNA.

21. The pegRNA of claim 19 or 20, wherein the pegRNA comprises an evopreq1 motif.

22. The pegRNA of claim 21, wherein the evopreq1 motif comprises the sequence of SEQ ID NO: 442, a sequence at least 80%, at least 85%, at least 90%, at least 95%, at least 96%, at least 97%, at least 98%, or at least 99% identical to the sequence of SEQ ID NO: 442, or a sequence having 1, 2, 3, 4, or 5 mutations relative to SEQ ID NO: 442.

23. The pegRNA of any one of claims 1-22, wherein the pegRNA comprises a UA flip in the pegRNA scaffold sequence.

24. The pegRNA of any one of claims 1-23, wherein the pegRNA comprises the sequence of any one of SEQ ID NOs: 454-815, or a sequence at least 80%, at least 85%, at least 90%, at least 95%, at least 96%, at least 97%, at least 98%, or at least 99% identical to the sequence of any one of SEQ ID NOs: 454-815.

25. A composition comprising (i) a prime editor comprising a nucleic acid programmable DNA binding protein (napDNAbp) and a DNA polymerase domain, and (ii) a pegRNA of any one of claims 1-24.

26. The composition of claim 25, wherein the prime editor comprises PE2, PE2max, PE3, PE3max, PE3b, or PE3bmax architecture.

27. The composition of claim 25 or 26, wherein the prime editor comprises a Cas9 protein.

28. The composition of claim 27, wherein the Cas9 protein comprises an inactivating mutation in an HNH nuclease domain.

29. The composition of claim 27 or 28, wherein the Cas9 protein comprises the amino acid sequence of SEQ ID NO: 6, or an amino acid sequence at least 70%, at least 75%, at least 80%, at least 85%, at least 90%, at least 95%, at least 96%, at least 97%, at least 98%, or at least 99% identical to the amino acid sequence of SEQ ID NO: 6.

30. The composition of any one of claims 27-29, wherein the Cas9 protein comprises a K775R substitution and/or a K918A substitution relative to the amino acid sequence of SEQ ID NO: 6.

31. The composition of any one of claims 27-29, wherein the Cas9 protein comprises a D23G substitution and/or an H754R substitution relative to the amino acid sequence of SEQ ID NO: 6.

32. The composition of any one of claims 25-31, wherein the DNA polymerase is a reverse transcriptase.

33. The composition of claim 32, wherein the reverse transcriptase is an MMLV reverse transcriptase.

34. The composition of claim 33, wherein the RNaseH domain of the MMLV reverse transcriptase is truncated relative to the full-length wild type MMLV reverse transcriptase.

35. The composition of claim 34, wherein the RNaseH domain of the MMLV reverse transcriptase is truncated between amino acids D497 and I498 relative to the amino acid sequence of SEQ ID NO: 33 or 34, or an amino acid sequence at least 70%, at least 75%, at least 80%, at least 85%, at least 90%, at least 95%, at least 96%, at least 97%, at least 98%, or at least 99% identical to the amino acid sequence of SEQ ID NO: 33 or 34.

36. The composition of any one of claims 33-35, wherein the MMLV reverse transcriptase comprises the amino acid substitution V223Y relative to the amino acid sequence of SEQ ID NO: 33 or 34.

37. The composition of any one of claims 33-36, wherein the MMLV reverse transcriptase comprises the amino acid sequence of SEQ ID NO: 33 or 34, or an amino acid sequence at least 70%, at least 75%, at least 80%, at least 85%, at least 90%, at least 95%, at least 96%, at least 97%, at least 98%, or at least 99% identical to the amino acid sequence of SEQ ID NO: 33 or 34.

38. The composition of any one of claims 25-36, wherein the reverse transcriptase is a Tf1 reverse transcriptase.

39. The composition of claim 38, wherein the Tf1 reverse transcriptase comprises the amino acid sequence of SEQ ID NO: 55, or an amino acid sequence at least 70%, at least 75%, at least 80%, at least 85%, at least 90%, at least 95%, at least 96%, at least 97%, at least 98%, or at least 99% identical to the amino acid sequence of SEQ ID NO: 55.

40. The composition of any one of claims 25-40 further comprising a nicking gRNA.

41. The composition of claim 40, wherein the spacer of the nicking gRNA comprises the nucleotide sequence:
(SEQ ID NO: 387)
GGCGGCTGAGGAAGCTGAGG;
(SEQ ID NO: 385)
GGCGGCGGCGGCGGCGGTGG;
(SEQ ID NO: 384)
GCTGCTGCTGCTGCTGCTGC;
(SEQ ID NO: 393)
GAAGGACTTGAGGGACTCGA;
(SEQ ID NO: 394)
GTGAGGAAGCTGAGGAGGCGG;
(SEQ ID NO: 395)
GCTGTTGCTGCTGCTGCTGC;
(SEQ ID NO: 383)
GCTGCTGCTGCTGCTGCTGGA;
(SEQ ID NO: 382)
GCTGCTGCTGGAAGGACTTG;
(SEQ ID NO: 396)
GGCCTTCATCAGCTTTTCC;
(SEQ ID NO: 397)
GGCTTTCATCAGCTTTTCC;
(SEQ ID NO: 398)
GGGACTCGAAGGCCTTCAT;
(SEQ ID NO: 399)
GGAAGGACTTGAGGGACTCG;
(SEQ ID NO: 400)
GCTGCTGCTGGAAGGACTT;
(SEQ ID NO: 401)
GCTGCTGCTGCTGCTGCTGG;
(SEQ ID NO: 402)
GCTGGAAGGACTTGAGGGACT;
(SEQ ID NO: 403)
GCTGCTGCTGCTGGAAGGACT;
(SEQ ID NO: 404)
CTGCTGCTGCTGCTGCTGGAA;
(SEQ ID NO: 405)
GCTGCTGCTGCTGCTGCTGCT;
(SEQ ID NO: 406)
GCTGCTGGAAGGACTTGAG;
(SEQ ID NO: 407)
GCTGCTGGAAGGACTTCAG;
(SEQ ID NO: 408)
GCTGCTGAAAGGACTTCAG;
or
(SEQ ID NO: 409)
GCTGTTGGAAGGACTTCAG,
or a nucleotide sequence at least 80%, at least 85%, at least 90%, at least 95%, at least 96%, at least 97%, at least 98%, or at least 99% identical to any of these SEQ ID NOs.

42. The composition of claim 40 or 41, wherein the spacer of the nicking gRNA comprises the nucleotide sequence GCTGCTGCTGCTGCTGCTGC (SEQ ID NO: 384), or a nucleotide sequence at least 80%, at least 85%, at least 90%, at least 95%, at least 96%, at least 97%, at least 98%, or at least 99% identical to SEQ ID NO: 384.

43. The composition of claim 40 or 41, wherein the spacer of the nicking gRNA comprises the nucleotide sequence GCTGCTGGAAGGACTTGAG (SEQ ID NO: 406), or a nucleotide sequence at least 80%, at least 85%, at least 90%, at least 95%, at least 96%, at least 97%, at least 98%, or at least 99% identical to SEQ ID NO: 406.

44. A method of treating Huntington's disease by prime editing comprising contacting a target nucleotide sequence with a composition of any one of claims 25-43.

45. The method of claim 44, wherein the contacting results in the contraction or replacement of a CAG repeat sequence in the target nucleotide sequence.

46. The method of claim 45, wherein the CAG repeat sequence is contracted from greater than 35 repeats to 35 or fewer repeats.

47. The method of claim 45, wherein the CAG repeat sequence is greater than 35 repeats in length and is replaced with a CAG repeat sequence of 35 or fewer repeats in length.

48. The method of any one of claims 45-47, wherein the CAG repeat sequence is contracted to four repeats or replaced with a sequence comprising four CAG repeats.

49. The method of any one of claims 44-48 further comprising nicking the non-PAM-containing strand of the target nucleotide sequence using a nicking gRNA.

50. The method of claim 49, wherein the spacer of the nicking gRNA comprises the nucleotide sequence:
(SEQ ID NO: 387)
GGCGGCTGAGGAAGCTGAGG;
(SEQ ID NO: 385)
GGCGGCGGCGGCGGCGGTGG;
(SEQ ID NO: 384)
GCTGCTGCTGCTGCTGCTGC;
(SEQ ID NO: 393)
GAAGGACTTGAGGGACTCGA;
(SEQ ID NO: 394)
GTGAGGAAGCTGAGGAGGCGG;
(SEQ ID NO: 395)
GCTGTTGCTGCTGCTGCTGC;
(SEQ ID NO: 383)
GCTGCTGCTGCTGCTGCTGGA;
(SEQ ID NO: 382)
GCTGCTGCTGGAAGGACTTG;
(SEQ ID NO: 396)
GGCCTTCATCAGCTTTTCC;
(SEQ ID NO: 397)
GGCTTTCATCAGCTTTTCC;
(SEQ ID NO: 398)
GGGACTCGAAGGCCTTCAT;
(SEQ ID NO: 399)
GGAAGGACTTGAGGGACTCG;
(SEQ ID NO: 400)
GCTGCTGCTGGAAGGACTT;
(SEQ ID NO: 401)
GCTGCTGCTGCTGCTGCTGG;
(SEQ ID NO: 402)
GCTGGAAGGACTTGAGGGACT;
(SEQ ID NO: 403)
GCTGCTGCTGCTGGAAGGACT;
(SEQ ID NO: 404)
CTGCTGCTGCTGCTGCTGGAA;
(SEQ ID NO: 405)
GCTGCTGCTGCTGCTGCTGCT;
(SEQ ID NO: 406)
GCTGCTGGAAGGACTTGAG;
(SEQ ID NO: 407)
GCTGCTGGAAGGACTTCAG;
(SEQ ID NO: 408)
GCTGCTGAAAGGACTTCAG;
or
(SEQ ID NO: 409)
GCTGTTGGAAGGACTTCAG,
or a nucleotide sequence at least 80%, at least 85%, at least 90%, at least 95%, at least 96%, at least 97%, at least 98%, or at least 99% identical to any of these SEQ ID NOs.

51. The method of claim 49 or 50, wherein the spacer of the nicking gRNA comprises the nucleotide sequence GCTGCTGCTGCTGCTGCTGC (SEQ ID NO: 384), or a nucleotide sequence at least 80%, at least 85%, at least 90%, at least 95%, at least 96%, at least 97%, at least 98%, or at least 99% identical to SEQ ID NO: 384.

52. The method of claim 49 or 50, wherein the spacer of the nicking gRNA comprises the nucleotide sequence GCTGCTGGAAGGACTTGAG (SEQ ID NO: 406), or a nucleotide sequence at least 80%, at least 85%, at least 90%, at least 95%, at least 96%, at least 97%, at least 98%, or at least 99% identical to SEQ ID NO: 406.

53. The method of any one of claims 44-52, wherein the contacting is performed in a cell.

54. The method of claim 53, wherein the cell is a eukaryotic cell.

55. The method of claim 53 or 54, wherein the cell is a human cell.

56. The method of any one of claims 53-55, wherein the cell is in vitro.

57. The method of any one of claims 53-55, wherein the cell is ex vivo.

58. The method of any one of claims 53-55, wherein the cell is in a subject.

59. The method of claim 58, wherein the subject is a human.

60. The method of any one of claims 53-59, wherein one or more polynucleotides encoding the composition are delivered to the cell.

61. The method of claim 60, wherein the one or more polynucleotides encoding the composition are delivered to the cell in one or more adeno-associated virus (AAV) particles.

62. The method of claim 61, wherein the one or more polynucleotides encoding the composition are delivered to the cell in two AAV particles.

63. The method of claim 61 or 62, wherein the AAV particles comprise AAV1, AAV2, AAV3, AAV4, AAV5, AAV6, AAV7, AAV8, or AAV9.

64. The method of any one of claims 61-63, wherein the AAV particles comprise AAV9.

65. The method of any one of claims 61-64, wherein a first and second AAV particle are delivered to the cell, wherein the first AAV particle comprises a polynucleotide comprising the structure 5′-[inverted terminal repeat (ITR) sequence]-[promoter]-[napDNAbp N-terminal fragment]-[N-intein]-[terminator sequence]-[ITR sequence]-3′, and wherein the second AAV particle comprises a polynucleotide comprising the structure 5′-[ITR sequence]-[promoter]-[C-intein]-[napDNAbp C-terminal fragment]-[reverse transcriptase]-[terminator sequence]-[optional nicking gRNA]-[pegRNA]-[ITR]-3′.

66. A polynucleotide encoding the pegRNA of any one of claims 1-24.

67. One or more polynucleotides encoding the prime editor and the pegRNA of the composition of any one of claims 25-43.

68. A vector comprising the polynucleotide of claim 66.

69. One or more vectors comprising the one or more polynucleotides of claim 67.

70. One or more AAV particles comprising the polynucleotide of claim 66, the one or more polynucleotides of claim 67, the vector of claim 68, or the one or more vectors of claim 69.

71. The one or more AAV particles of claim 70, wherein the AAV particles comprise AAV1, AAV2, AAV3, AAV4, AAV5, AAV6, AAV7, AAV8, or AAV9.

72. The one or more AAV particles of claim 70 or 71, wherein the AAV particles comprise AAV9.

73. The one or more AAV particles of any one of claims 70-72 comprising a first AAV particle and a second AAV particle, wherein the first AAV particle comprises a polynucleotide comprising the structure 5′-[inverted terminal repeat (ITR) sequence]-[promoter]-[napDNAbp N-terminal fragment]-[N-intein]-[terminator sequence]-[ITR sequence]-3′, and wherein the second AAV particle comprises a polynucleotide comprising the structure 5′-[ITR sequence]-[promoter]-[C-intein]-[napDNAbp C-terminal fragment]-[reverse transcriptase]-[terminator sequence]-[optional nicking gRNA]-[pegRNA]-[ITR]-3′.

74. A pharmaceutical composition comprising the pegRNA of any one of claims 1-24, the composition of any one of claims 25-43, the one or more polynucleotides of claim 66 or 67, the one or more vectors of claim 68 or 69, or the one or more AAV particles of any one of claims 70-73.

75. A cell comprising the pegRNA of any one of claims 1-24, the composition of any one of claims 25-43, the one or more polynucleotides of claim 66 or 67, the one or more vectors of claim 68 or 69, or the one or more AAV particles of any one of claims 70-73.

76. A kit comprising the pegRNA of any one of claims 1-24, the composition of any one of claims 25-43, the one or more polynucleotides of claim 66 or 67, the one or more vectors of claim 68 or 69, or the one or more AAV particles of any one of claims 70-73.

77. Use of the pegRNA of any one of claims 1-24, the composition of any one of claims 25-43, the one or more polynucleotides of claim 66 or 67, the one or more vectors of claim 68 or 69, the one or more AAV particles of any one of claims 70-73, or the pharmaceutical composition of claim 74 for the treatment of Huntington's disease.

78. Use of the pegRNA of any one of claims 1-24, the composition of any one of claims 25-43, the one or more polynucleotides of claim 66 or 67, the one or more vectors of claim 68 or 69, the one or more AAV particles of any one of claims 70-73, or the pharmaceutical composition of claim 74 in the manufacture of a medicament for the treatment of Huntington's disease.

79. A pegRNA comprising a spacer sequence comprising the nucleic acid sequence:
(SEQ ID NO: 388)
GCAAGACTAACCTGGCCAACA;
(SEQ ID NO: 389)
GTCCGGAGTTCAAGACTAACC;
(SEQ ID NO: 390)
GAAGGTGGATCACCTGAGGTC;
(SEQ ID NO: 391)
GTCTGGAGTAGCTGGGATTAC;
or
(SEQ ID NO: 392)
GCAGGCGCGCGACACCACGCC,
or a nucleic acid sequence at least 80%, at least 85%, at least 90%, at least 95%, at least 96%, at least 97%, at least 98%, or at least 99% identical to any one of SEQ ID NOs: 388-392.

80. The pegRNA of claim 79, wherein the pegRNA comprises the spacer sequence GCAAGACTAACCTGGCCAACA (SEQ ID NO: 388), or a spacer sequence at least 80%, at least 85%, at least 90%, at least 95%, at least 96%, at least 97%, at least 98%, or at least 99% identical to SEQ ID NO: 388.

81. The pegRNA of claim 79 or 80, wherein the pegRNA comprises a primer binding site (PBS) of about 8 to about 14 nucleotides in length.

82. The pegRNA of any one of claims 79-81, wherein the pegRNA comprises a PBS of about 8, about 9, about 10, about 11, about 12, about 13, or about 14 nucleotides in length.

83. The pegRNA of any one of claims 79-82, wherein the pegRNA comprises a PBS of about 10 nucleotides in length.

84. The pegRNA of any one of claims 79-83, wherein the pegRNA comprises a reverse transcription template comprising a homology arm of about 8 to about 50 nucleotides in length, wherein the homology arm is complementary to a sequence directly downstream of a GAA repeat in a wildtype FXN gene.

85. The pegRNA of any one of claims 79-84, wherein the homology arm is about 40 nucleotides in length.

86. The pegRNA of any one of claims 79-85, wherein the pegRNA is an engineered pegRNA (epegRNA).

87. The pegRNA of any one of claims 79-86, wherein the pegRNA comprises at its 3′ end a structural motif that improves stability of the pegRNA.

88. The pegRNA of claim 87, wherein the pegRNA comprises an evopreq1 motif.

89. The pegRNA of claim 88, wherein the evopreq1 motif comprises the sequence of SEQ ID NO: 442, a sequence at least 80%, at least 85%, at least 90%, at least 95%, at least 96%, at least 97%, at least 98%, or at least 99% identical to the sequence of SEQ ID NO: 442, or a sequence having 1, 2, 3, 4, or 5 mutations relative to SEQ ID NO: 442.

90. The pegRNA of any one of claims 79-89, wherein the pegRNA comprises a UA flip in the pegRNA scaffold sequence.

91. The pegRNA of any one of claims 79-90, wherein the pegRNA comprises a gRNA core sequence selected from the group consisting of SEQ ID NOs: 393-413.

92. A composition comprising (i) a prime editor comprising a nucleic acid programmable DNA binding protein (napDNAbp) and a DNA polymerase domain, and (ii) a pegRNA of any one of claims 79-91.

93. The composition of claim 92, wherein the prime editor comprises PE2, PE2max, PE3, PE3max, PE3b, or PE3bmax architecture.

94. The composition of claim 92 or 93, wherein the prime editor comprises a Cas9 protein.

95. The composition of claim 94, wherein the Cas9 protein comprises an inactivating mutation in an HNH domain.

96. The composition of claim 94 or 95, wherein the Cas9 protein comprises the amino acid sequence of SEQ ID NO: 6, or an amino acid sequence at least 70%, at least 75%, at least 80%, at least 85%, at least 90%, at least 95%, at least 96%, at least 97%, at least 98%, or at least 99% identical to the amino acid sequence of SEQ ID NO: 6.

97. The composition of any one of claims 94-96, wherein the Cas9 protein comprises a K775R substitution and/or a K918A substitution relative to the amino acid sequence of SEQ ID NO: 6.

98. The composition of any one of claims 94-96, wherein the Cas9 protein comprises a D23G substitution and/or an H754R substitution relative to the amino acid sequence of SEQ ID NO: 6.

99. The composition of any one of claims 92-98, wherein the DNA polymerase is a reverse transcriptase.

100. The composition of claim 99, wherein the reverse transcriptase is an MMLV reverse transcriptase.

101. The composition of claim 100, wherein the RNaseH domain of the MMLV reverse transcriptase is truncated relative to the full-length wild type MMLV reverse transcriptase.

102. The composition of claim 101, wherein the RNaseH domain of the MMLV reverse transcriptase is truncated between amino acids D497 and I498 relative to the amino acid sequence of SEQ ID NO: 33 or 34, or an amino acid sequence at least 70%, at least 75%, at least 80%, at least 85%, at least 90%, at least 95%, at least 96%, at least 97%, at least 98%, or at least 99% identical to the amino acid sequence of SEQ ID NO: 33 or 34.

103. The composition of claim 101 or 102, wherein the MMLV reverse transcriptase comprises the amino acid substitution V223Y relative to the amino acid sequence of SEQ ID NO: 33 or 34.

104. The composition of any one of claims 100-103, wherein the MMLV reverse transcriptase comprises the amino acid sequence of SEQ ID NO: 33 or 34, or an amino acid sequence at least 70%, at least 75%, at least 80%, at least 85%, at least 90%, at least 95%, at least 96%, at least 97%, at least 98%, or at least 99% identical to the amino acid sequence of SEQ ID NO: 33 or 34.

105. The composition of claim 99, wherein the reverse transcriptase is a Tf1 reverse transcriptase.

106. The composition of claim 105, wherein the Tf1 reverse transcriptase comprises the amino acid sequence of SEQ ID NO: 55, or an amino acid sequence at least 70%, at least 75%, at least 80%, at least 85%, at least 90%, at least 95%, at least 96%, at least 97%, at least 98%, or at least 99% identical to the amino acid sequence of SEQ ID NO: 55.

107. The composition of any one of claims 92-106 further comprising a nicking gRNA.

108. The composition of claim 107, wherein the spacer of the nicking gRNA comprises the nucleotide sequence:
(SEQ ID NO: 410)
GTCCCAAAGTGCTGAGATTAT;
(SEQ ID NO: 411)
GTGTATTTTTTAGTAGATACT;
(SEQ ID NO: 412)
GATTCTCCTGCCGCAGCCTC;
or
(SEQ ID NO: 413)
GCGACACCACGCCCGGCTAAC,
or a nucleotide sequence at least 80%, at least 85%, at least 90%, at least 95%, at least 96%, at least 97%, at least 98%, or at least 99% identical to any one of SEQ ID NOs: 410-413.

109. The composition of claim 107 or 108, wherein the spacer of the nicking gRNA comprises the nucleotide sequence GTGTATTTTTTAGTAGATACT (SEQ ID NO: 411), or a nucleic acid sequence at least 80%, at least 85%, at least 90%, at least 95%, at least 96%, at least 97%, at least 98%, or at least 99% identical to SEQ ID NO: 411.

110. The composition of claim 107 or 108, wherein the spacer of the nicking gRNA comprises the nucleotide sequence GCGACACCACGCCCGGCTAAC (SEQ ID NO: 413), or a nucleic acid sequence at least 80%, at least 85%, at least 90%, at least 95%, at least 96%, at least 97%, at least 98%, or at least 99% identical to SEQ ID NO: 413.

111. A method of treating Friedreich's ataxia by prime editing comprising contacting a target nucleotide sequence with a composition of any one of claims 92-110.

112. The method of claim 111, wherein the contacting results in the deletion of a GAA repeat sequence in the target nucleotide sequence.

113. The method of claim 112, wherein the GAA repeat sequence deleted comprises greater than 65 GAA repeats.

114. The method of any one of claims 111-113 further comprising nicking the non-PAM-containing strand of the target nucleotide sequence using a nicking gRNA.

115. The method of claim 114, wherein the spacer of the nicking gRNA comprises the nucleotide sequence:
(SEQ ID NO: 410)
GTCCCAAAGTGCTGAGATTAT;
(SEQ ID NO: 411)
GTGTATTTTTTAGTAGATACT;
(SEQ ID NO: 412)
GATTCTCCTGCCGCAGCCTC;
or
(SEQ ID NO: 413)
GCGACACCACGCCCGGCTAAC,
or a nucleotide sequence at least 80%, at least 85%, at least 90%, at least 95%, at least 96%, at least 97%, at least 98%, or at least 99% identical to any one of SEQ ID NOs: 410-413.

116. The method of claim 114 or 115, wherein the spacer of the nicking gRNA comprises the nucleotide sequence GTGTATTTTTTAGTAGATACT (SEQ ID NO: 411), or a nucleic acid sequence at least 80%, at least 85%, at least 90%, at least 95%, at least 96%, at least 97%, at least 98%, or at least 99% identical to SEQ ID NO: 411.

117. The method of claim 114 or 115, wherein the spacer of the nicking gRNA comprises the nucleotide sequence GCGACACCACGCCCGGCTAAC (SEQ ID NO: 413), or a nucleic acid sequence at least 80%, at least 85%, at least 90%, at least 95%, at least 96%, at least 97%, at least 98%, or at least 99% identical to SEQ ID NO: 413.

118. The method of any one of claims 111-117, wherein the contacting is performed in a cell.

119. The method of claim 118, wherein the cell is a eukaryotic cell.

120. The method of claim 118 or 119, wherein the cell is a human cell.

121. The method of any one of claims 118-120, wherein the cell is in vitro.

122. The method of any one of claims 118-120, wherein the cell is ex vivo.

123. The method of any one of claims 118-122, wherein the cell is in a subject.

124. The method of claim 123, wherein the subject is a human.

125. The method of any one of claims 118-124, wherein one or more polynucleotides encoding the composition are delivered to the cell.

126. The method of claim 125, wherein the one or more polynucleotides encoding the composition are delivered to the cell in one or more adeno-associated virus (AAV) particles.

127. The method of claim 126, wherein the one or more polynucleotides encoding the composition are delivered to the cell in two AAV particles.

128. The method of claim 126 or 127, wherein the AAV particles comprise AAV1, AAV2, AAV3, AAV4, AAV5, AAV6, AAV7, AAV8, or AAV9.

129. The method of any one of claims 126-128, wherein the AAV particles comprise AAV9.

130. The method of any one of claims 126-129, wherein a first and second AAV particle are delivered to the cell, wherein the first AAV particle comprises a polynucleotide comprising the structure 5′-[inverted terminal repeat (ITR) sequence]-[promoter]-[napDNAbp N-terminal fragment]-[N-intein]-[terminator sequence]-[ITR sequence]-3′, and wherein the second AAV particle comprises a polynucleotide comprising the structure 5′-[ITR sequence]-[promoter]-[C-intein]-[napDNAbp C-terminal fragment]-[reverse transcriptase]-[terminator sequence]-[optional nicking gRNA]-[pegRNA]-[ITR]-3′.

131. A polynucleotide encoding the pegRNA of any one of claims 79-91.

132. One or more polynucleotides encoding the prime editor and the pegRNA of the composition of any one of claims 92-110.

133. A vector comprising the polynucleotide of claim 131.

134. One or more vectors comprising the one or more polynucleotides of claim 132.

135. One or more AAV particles comprising the polynucleotide of claim 131, the one or more polynucleotides of claim 132, the vector of claim 133, or the one or more vectors of claim 134.

136. The one or more AAV particles of claim 135, wherein the AAV particles comprise AAV1, AAV2, AAV3, AAV4, AAV5, AAV6, AAV7, AAV8, or AAV9.

137. The one or more AAV particles of claim 135 or 136, wherein the AAV particles comprise AAV9.

138. The one or more AAV particles of any one of claims 135-137 comprising a first AAV particle and a second AAV particle, wherein the first AAV particle comprises a polynucleotide comprising the structure 5′-[inverted terminal repeat (ITR) sequence]-[promoter]-[napDNAbp N-terminal fragment]-[N-intein]-[terminator sequence]-[ITR sequence]-3′, and wherein the second AAV particle comprises a polynucleotide comprising the structure 5′-[ITR sequence]-[promoter]-[C-intein]-[napDNAbp C-terminal fragment]-[reverse transcriptase]-[terminator sequence]-[optional nicking gRNA]-[pegRNA]-[ITR]-3′.

139. A pharmaceutical composition comprising the pegRNA of any one of claims 79-91, the composition of any one of claims 92-110, the one or more polynucleotides of claim 131 or 132, the one or more vectors of claim 133 or 134, or the one or more AAV particles of any one of claims 135-138.

140. A cell comprising the pegRNA of any one of claims 79-91, the composition of any one of claims 92-110, the one or more polynucleotides of claim 131 or 132, the one or more vectors of claim 133 or 134, or the one or more AAV particles of any one of claims 135-138.

141. A kit comprising the pegRNA of any one of claims 79-91, the composition of any one of claims 92-110, the one or more polynucleotides of claim 131 or 132, the one or more vectors of claim 133 or 134, or the one or more AAV particles of any one of claims 135-138.

142. Use of the pegRNA of any one of claims 79-91, the composition of any one of claims 92-110, the one or more polynucleotides of claim 131 or 132, the one or more vectors of claim 133 or 134, the one or more AAV particles of any one of claims 135-138, or the pharmaceutical composition of claim 139 in the treatment of Friedreich's ataxia.

143. Use of the pegRNA of any one of claims 79-91, the composition of any one of claims 92-110, the one or more polynucleotides of claim 131 or 132, the one or more vectors of claim 133 or 134, the one or more AAV particles of any one of claims 135-138, or the pharmaceutical composition of claim 139 in the manufacture of a medicament for the treatment of Friedreich's ataxia.

144. A system for prime editing the HTT gene comprising:
a) a pegRNA comprising i) a spacer sequence comprising the sequence GACCCTGGAAAAGCTGAT (SEQ ID NO: 873), and ii) a reverse transcription template comprising an edit template comprising the sequence CAGCAGCAGCAG (SEQ ID NO: 874), CAGCAGCAGCAGCAGCAGCAGCAGCAG (SEQ ID NO: 875), or CAGCAGCAGCAGCAACAACAA (SEQ ID NO: 876);
b) a PE3bmax prime editor, optionally wherein the PE3bmax prime editor comprises the amino acid substitutions D23G and H754R within a Cas9 protein and V223Y within an MMLV reverse transcriptase; and
c) a nicking guide RNA comprising a spacer sequence comprising the sequence GCTGCTGCTGCTGCTGCTGC (SEQ ID NO: 384) or GCTGCTGGAAGGACTTGAG (SEQ ID NO: 406).

145. A system for prime editing the FXN gene comprising:
a) a pegRNA comprising i) a spacer sequence comprising the sequence GCAAGACTAACCTGGCCAACA (SEQ ID NO: 388);
b) a PE3bmax prime editor, optionally wherein the PE3bmax prime editor comprises the amino acid substitution V223Y within an MMLV reverse transcriptase; and
c) a nicking guide RNA comprising a spacer sequence comprising the sequence GTGTATTTTTTAGTAGATACT (SEQ ID NO: 411) or GCGACACCACGCCCGGCTAAC (SEQ ID NO: 413).

146. The method of any one of claim 60-65 or 125-130, wherein the composition is delivered to the brain of a subject.

147. The method of any one of claim 60-65, 125-130, or 146, wherein the composition is delivered by intracerebroventricular injection (ICV).
